(19) United States
(12) Patent Application Publication
(10) Pub. No.: US 2026/0092061 A1
(43) Pub. Date: Apr. 2, 2026

(54) COMPOUNDS FOR TARGETED PROTEIN DEGRADATION

(71) Applicant: Amphista Therapeutics Limited, Cambridge (GB)

(72) Inventors: David MCGARRY, Cambridge (GB); Gregor MEIER, Cambridge (GB); James OSBORNE, Cambridge (GB); Andrea TESTA, Cambridge (GB); Giles Albert BROWN, Cambridge (GB); Martin AMBLER, Cambridge (GB); Charlene FALLAN, Cambridge (GB)

(21) Appl. No.: 19/110,659

(22) PCT Filed: Sep. 13, 2023
(86) PCT No.: PCT/GB2023/052374

§ 371 Date: Mar. 11, 2025

(30) Foreign Application Priority Data
Sep. 13, 2022 (GB) 2213365.6
Dec. 20, 2022 (GB) 2219287.6
Jun. 16, 2023 (GB) 2309103.6

(51) Int. Cl. C07D 471/04; A61K 31/4365; A61K 31/4439; A61K 31/444; A61K 31/4545; A61K 31/4725; A61K 31/496; A61K 31/498; A61K 31/499; A61K 31/4995; A61K 31/506; A61K 31/5377; A61K 31/55; A61K 31/551; C07D 213/64; C07D 401/10; C07D 401/12; C07D 401/14; C07D 405/14; C07D 413/14; C07D 417/12; C07D 417/14; C07D 471/10; C07D 487/08; C07D 487/10; C07D 495/04; C07D 519/00; G01N 33/50

(52) U.S. Cl. CPC C07D 471/04; A61K 31/4365; A61K 31/4439; A61K 31/444; A61K 31/4545; A61K 31/4725; A61K 31/496; A61K 31/498; A61K 31/499; A61K 31/4995; A61K 31/506; A61K 31/5377; A61K 31/55; A61K 31/551; C07D 213/64; C07D 401/10; C07D 401/12; C07D 401/14; C07D 405/14; C07D 413/14; C07D 417/12; C07D 417/14; C07D 471/10; C07D 487/08; C07D 487/10; C07D 495/04; C07D 519/00; G01N 33/5038

(57) ABSTRACT

The present disclosure relates to a novel class of bifunctional molecules that are useful in a targeted or selective degradation of a protein.

### FIELD

The present disclosure relates to degradation of the Bromodomain-containing protein 9 (BRD9) protein. BRD9 has been linked to the proliferation of cancers, and the present disclosure relates to treatment of cancers, for example by BRD9 degradation. Specifically, the present disclosure relates to a novel class of bifunctional molecules that are useful in a targeted or selective degradation of BRD9, together with methods of preparing such molecules and therapeutic uses thereof. The present disclosure further relates to methods of treating cancer comprising the selective and/or targeted degradation of BRD9.

### BACKGROUND

BRD9 is a protein encoded by the BRD9 gene on chromosome 5. BRD9 is a component of the BAF (BRG1- or BRM-associated factors) complex, a SWI/SNF ATPase chromatin remodeling complex, and belongs to family IV of the bromodomain-containing proteins (D. Hay et al., Med. Chem. Commun., 2015, 6, 1381-1386). SWI/SNF uses the energy of ATP hydrolysis to remodel chromatin and mobilize nucleosomes. SWI/SNF is implicated in activating transcription by remodelling nucleosomes, thereby permitting increased access of transcription factors for their binding sites. It is also required for transcriptional repression of some genes, and so controls transcription in various ways.

Recurrent inactivating mutations in certain subunits of SWI/SNF complex have been identified in different cancers. Despite its known roles in tumour suppression, the mammalian SWI/SNF complex has recently received attention as a potential target for therapeutic inhibition (L. J. Martin et al., J. Med. Chem., 2016, 59, 4462-4475).

Studies have shown that BRD9 is preferentially used by cancers that harbour SMARCB1 abnormalities such as malignant rhabdoid tumors and several specific types of sarcoma (X. Zhu, Y. Liao and L. Tang, Onco Targets Ther., 2020, 13, 13191-13200). BRD9-containing complexes bind to both active promoters and enhancers, where they contribute to gene expression. Loss of BRD9 results in gene expression changes related to apoptosis regulation, translation, and development regulation. BRD9 is essential for the proliferation of SMARCB1-deficient cancer cell lines, suggesting it is a therapeutic target for these lethal cancers. (Xiaofeng Wang et. al., Nature Communications, 2019, 10 (1881)). Recent studies highlight a role of BRD9 in leukemia growth: BRD9 was shown to be required for the proliferation of acute myeloid leukemia (AML) cells (Nature Chemical Biology, 2016, 101038/nchembio.2115). In addition to the role of BRD9 as a functional dependency in certain cancers, BRD9 also plays a pivotal role in immune cells as a regulator of regulatory T cells (Tregs) via transcriptional control of Foxp3 target genes, “BioRxiv, 10.1101/2020.02.26.964981.

Because of BRD9's role in cancer proliferation there has been interest in the development of BRD9 inhibitors for the treatment of cancers including those described in: NO 2014/114721, WO 2016/077375, WO 2016/077378, WO 2016/139361, NO 2019/152440, a paper by Martin L. J. et. al., (Journal of Medicinal Chemistry 2016, 59, 4462-4475) titled “Structure-Based Design of an in Vivo Active Selective BRD9 Inhibitor”; a paper by Theodoulou N. H. et al., (Journal of Medicinal Chemistry 2015, 59, 1425-1439) titled “Discovery of I-BRD9, a selective Cell Active Chemical Probe for Bromodomain Containing Protein 9 Inhibition”; and a paper by Clack P. et. al., (Angewandte Chemie, 2015, 127, 6315-6319).

Targeted Protein Degradation (TPD) is a therapeutic modality, which relies on the use of synthetic molecules to repurpose cellular degradation machinery to induce degradation of specific disease-causing proteins. TPD approaches offer a number of advantages over other drug modalities (e.g. small molecule inhibitors, antibodies & protein-based agents, antisense oligonucleotides & related knockdown approaches) including: potentiated pharmacology due to catalytic protein removal from within cells; ability to inhibit multiple functions of a specific drug target including e.g. scaffolding function through target knockdown; opportunity for systemic dosing with good biodistribution; potent in vivo efficacy due to catalytic potency and long duration of action limited only by de novo protein resynthesis; and facile chemical synthesis and formulation using application of small molecule processes.

The majority of physiologic post-translational regulation of protein levels as well as removal of damaged, misfolded, or excess proteins is mediated by the ubiquitin-proteasome system (UPS). The UPS can be repurposed to degrade specific proteins using bifunctional chemical molecules as therapeutic agents, which act by inducing the proximity of desired substrates with UPS proteins to initiate a cascade of events which ultimately lead to degradation, and removal from the cell, of the desired targets by the proteasome.

Proteolysis targeting chimeras (PROTACs) constitute one such class of bifunctional degraders, which induce proximity of target proteins to the UPS by recruitment of specific ubiquitin E3 ligases. PROTACs are composed of two ligands joined by a linker—one ligand to engage a desired target protein and another ligand to recruit a ubiquitin E3 ligase.

The E3 ligases used most frequently in PROTACs are von Hippel-Lindau (VHL) and Cereblon (CRBN). PROTACs recruiting VHL are typically based on hydroxyproline-containing ligands, whereas PROTACs recruiting CRBN are typically characterised by the presence of a glutarimide moiety, such as thalidomide, pomalidomide and lenalidomide or close analogues to act as the warhead. Other ligases including mdm2 and the IAP family have also shown utility in PROTAC design.

However, these approaches suffer from a range of limitations, which restrict their utility to treat a wide range of diseases. For example, limitations of current PROTAC approaches include: inability to efficiently degrade some targets; poor activity of PROTACs in many specific cells due to low and variable expression of E3 ligases and other proteins required for efficient degradation; chemical properties which make it more difficult to prepare degraders with suitable drug-like properties including good drug metabolism & pharmacokinetic profiles; and high susceptibility to induced resistance mechanisms in tumours.

Because of these limitations, there remains a need to identify novel degrading mechanisms and warheads able to deliver new bifunctional degrader molecules, which show efficient degradation across a range of targets and cellular systems and/or with improved profiles suitable for drug development. Further bifunctional degrader molecules have been described in WO 2019/238886, WO 2019/238817, WO 2019/238816 and WO 2022/129925.

Protein degrading compounds that have an E3 ligase binding portion and a BRD9 binding portion wherein the BRD9 binding ligand binds to BRD9 and brings it to the ligase for ultimate degradation by the proteasome are described in Ciulli et al, (J. Med. Chem. 2019, 62, 2, 699 to 726), WO 2017/223452, WO 2019/152440, WO 2019/246423, WO 2019/246430, WO 2020/051235, WO 2020/106915, WO 2020/160192, WO 2020/160193, WO 2020/160196, WO 2021/022163, WO 2021/178920, WO 2020/160198, and WO 2020/160196.

Most of the known BRD9 inhibitors possess poor potency. Due to the important role BRD9 plays in cancer, there remains a need to identify bifunctional degrader molecules, which show efficient BRD9 degradation across a range of cellular systems and/or with improved profiles suitable for drug development.

### SUMMARY

The present disclosure is based on the identification of a novel class of bifunctional molecules that are useful in a targeted and/or selective degradation of BRD9. In particular, the present disclosure provides bifunctional molecules comprising a BRD9 binding ligand and a “warhead”, which facilitate proteasomal degradation of BRD9.

The removal and/or reduction of BRD9 from a cell or subject in need thereof, by means of a targeted protein degradation mechanism may find particular application in therapy, for example, the treatment of cancers. Thus, the present disclosure further relates to methods of treating cancer comprising the selective and/or targeted degradation of BRD9, and also bifunctional molecules and pharmaceutical compositions for use in such methods.

The bifunctional molecules described herein comprise a general structure of:

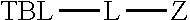

wherein TBL is a target protein binding ligand that binds to BRD9 and L is a linker. The moiety “Z” (a “warhead”) modulates, facilitates and/or promotes proteasomal degradation of the target protein BRD9 and may, in some cases, be referred to as a modulator, facilitator and/or promoter of proteasomal degradation. For example, in use, the TBL moiety of the bifunctional molecule binds to BRD9. The moiety Z (which is joined or otherwise connected to the TBL via the linker) then modulates, facilitates and/or promotes the degradation of BRD9, e.g. by acting to bring the BRD9 protein into proximity with a proteasome and/or by otherwise causing the BRD9 protein to be marked for proteasomal degradation within a cell.

Thus, the bifunctional molecules described in the present disclosure may be considered to comprise: a target protein binding ligand (TBL) that binds to BRD9 (i.e. a ligand capable of binding (e.g. specifically binding) to BRD9; a warhead or degradation tag (Z) (e.g. moiety Z which acts to modulate, facilitate and/or promote the degradation of this target protein) and a linker (e.g. a chemical linker) which conjugates, joins or connects TBL and Z.

The bifunctional molecules described in the present disclosure have been shown to be effective degraders of BRD9. Without being bound by theory, it is hypothesised that the Z moiety of the bifunctional molecules described herein does not bind to the particular E3 ligases typically relied on in the classical PROTAC approaches discussed above (such as CRBN and VHL). Accordingly, the bifunctional molecules described herein are believed to modulate, facilitate and/or promote proteasomal degradation via an alternative mechanism. Thus, the present class of bifunctional molecules may be useful against a wider range of diseases (including those that are resistant to many PROTAC degraders).

The bifunctional molecules described herein may provide degraders with one or more properties that will facilitate, enhance and/or promote their use in vivo (e.g. one or more drug-like properties). In particular, bifunctional molecules comprising the warhead Z may offer improvements in levels of bioavailability (e.g. oral bioavailability) over many classical PROTAC degraders. Additionally, or alternatively, bifunctional molecules comprising the warhead Z may provide improved levels of CNS (central nervous system) penetration (in contrast to many other degrader molecules currently known in the art).

The bifunctional molecules described in the present disclosure are particularly designed to degrade BRD9. In particular, the present inventors have identified that attachment of a BRD9 binding ligand to a linker that is itself attached to a warhead forms a bifunctional molecule capable of degrading BRD9. Furthermore, the present inventors have identified that these bifunctional molecules can be used to provide a particularly selective degradation of BRD9 over other types of BRD protein (e.g. BRD4 and/or BRD7), whilst also maintaining good levels of degradation.

According to a first aspect of the disclosure there is provided a bifunctional molecule comprising the general formula:

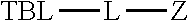

wherein TBL is a target protein binding ligand that binds BRD9;
L is a linker; and
Z comprises a structure according to formula (I):

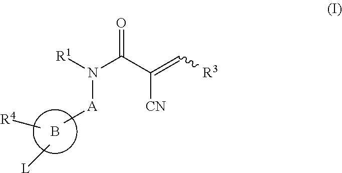

wherein
R¹ is selected from C₁ to C₆ alkyl, benzyl, substituted benzyl, carbocyclyl, substituted carbocyclyl, heterocyclyl and substituted heterocyclyl, optionally wherein the C₁ to C₆ alkyl is substituted with one or more heteroatoms selected from halo, N, O and S and/or is substituted with a carbocyclic or heterocyclic group;
A is absent or is CR²R^{2′};
B is selected from aryl, heteroaryl, substituted aryl and substituted heteroaryl;
R² and R^{2′} are each independently selected from H and C₁ to C₆ alkyl, optionally wherein the C₁ to C₆ alkyl is substituted with one or more heteroatoms selected from N, O, S or halo, or wherein R² and R^{2′} together form an optionally substituted 3-, 4-, 5- or 6-membered carbocyclic or heterocyclic ring;
R³ is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S;
R⁴ is H, C₁ to C₆ alkyl, optionally wherein the C₁ to C₆ alkyl is substituted with one or more heteroatoms selected from N, O or S;
or wherein R¹ and R⁴ together form an optionally substituted 5-, 6-, or 7-membered heterocyclic ring;
or wherein when A is CR²R^{2′};
R¹ and R² together form an optionally substituted 5-, 6-, or 7-membered heterocyclic ring; or
R² and R⁴ together form an optionally substituted 5-, 6-, or 7-membered heterocyclic or carbocyclic ring;
wherein L shows the point of attachment of the linker;
or
Z comprises a structure according to formula (WZI):

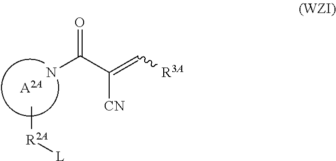

wherein:
ring A^{2A} is an optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from N, O and S;
R^{2A} is absent or is selected from aryl, substituted aryl, heteroaryl, substituted heteroaryl, heterocycloalkyl, substituted heterocycloalkyl, NR^{y}, —CH(aryl)-, —CH(substituted aryl)-, —CH(heteroaryl)- and —CH(substituted heteroaryl)-;
wherein R^{y} is optionally substituted C₁₋₆alkyl or H;
R^{3A} is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S; and
L shows the point of attachment of the linker;
or
Z comprises a structure according to formula (WI):

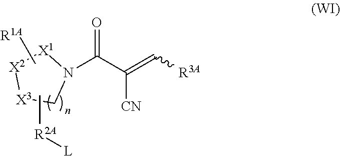

wherein R^{1A} is absent or is selected from aryl, substituted aryl, heteroaryl, substituted heteroaryl, cycloalkyl, C₁ to C₆ alkyl and substituted C₁ to C₆ alkyl;
R^{2A} is absent or is selected from aryl, substituted aryl, heteroaryl, substituted heteroaryl, heterocycloalkyl, substituted heterocycloalkyl, —CH(aryl)-, —CH(substituted aryl)-, —CH(heteroaryl)- and —CH(substituted heteroaryl)-;
R^{3A} is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S;
X¹ is CH₂;
X² and X³ are each independently CH₂, or a heteroatom selected from O and NR^{x}, wherein R^{x} is H or C₁ to C₆ alkyl; and
n is 0, 1, 2, or 3; and
L shows the point of attachment of the linker;
or
   Z comprises a structure according to formula (A):

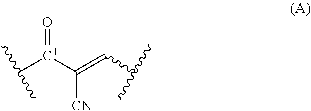

wherein the linker is attached to carbonyl carbon C¹;
in particular, wherein Z consists of, or consists essentially of, a structure according to formula (A1):

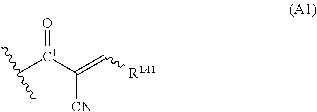

wherein:
R^{1A1} is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S; and
wherein the linker is attached to carbonyl carbon C¹.

In some examples, the BRD9 binder is of formula 1a:

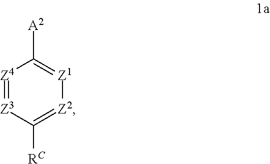

wherein:
Z¹ is N or CR^{A};
Z² is N or CR^{B};
Z³ is N or CR^{D};
Z⁴ is N or CR^{E};
wherein no more than 3 of Z¹, Z², Z³ and Z⁴ are N;
R^{A} and R^{E} are each independently selected from the group consisting of —H, —O—C₁₋₃alkyl and —C₁₋₃alkyl;
R^{B} and R^{D} are each independently selected from the group consisting of —O—C₁₋₃alkyl, —H, —OH, halogen, —NH₂, —C₁₋₃-alkyl, —O—C₁₋₃haloalkyl, —C₁₋₃alkyl-O—C₁₋₃alkyl, 4-7 membered heterocycloalkyl, —C₁₋₃alkyl-SO₂—C₁₋₃alkyl, —C₁₋₃alkyl-NH₂, —C₁₋₃alkyl-N(—C₁₋₃alkyl)₂, —N(C₁₋₃alkyl)₂, —NH—R^{F};
R^{F} is selected from —SO₂—C₁₋₃alkyl and —C₁₋₃alkyl, wherein the —C₁₋₃alkyl is optionally substituted with a 5 to 6 membered heteroaryl;
alternatively, R^{A} and R^{B} taken together form a benzene ring;
alternatively, R^{C} and Z² or R^{C} and Z³ taken together (e.g. R^{C} and R^{B} or R^{C} and R^{D} taken together with the carbon atoms to which they are joined) form a 5-7 membered heterocycloalkyl optionally substituted with —C₁₋₃alkyl;
R^{C} is selected from the group consisting of —H, —Y—R^{G}, —NH₂, —C₁₋₃alkyl and 4-7 membered heterocycloalkyl;
Y is absent or is selected from the group consisting of —CR^{H}R^{I}—, —SO₂— and —CO—;
R^{H} and R^{I} are each independently selected from —H or —C₁₋₃alkyl; or R^{H} and R^{I} taken together form a —C₃₋₄cycloalkyl,
R^{G} is selected from the group consisting of —NH₂, —OH, —C₁₋₃alkyl, —N(R^{J}R^{K}), —O—R^{L}, aryl, 5-6 membered heteroaryl, wherein the aryl and heteroaryl are optionally and independently substituted with one or more halogen, optionally substituted 4- to 7-membered monocyclic heterocycloalkyl, and optionally substituted 7- to 12-membered bicyclic heterocycloalkyl, which monocylic or bicyclic heterocycloalkyl are optionally substituted with any suitable substituent, such as one or more groups independently selected from halogen, —OH, —NH₂, —C₁₋₃alkyl, —NHC₁₋₃alkyl, —N(C₁₋₃alkyl)₂, —O—C₁₋₃alkyl and —CH₂—R^{M1};
R^{M1} is selected from 5-10 membered mono- or bicyclic aryl or heteroaryl, which is optionally substituted with —NH₂, —OH, halogen, —CN, C₁₋₃alkyl, —O—C₁₋₃alkyl;
R^{J} is —H or —C₁₋₃alkyl;
R^{K} is selected from the group consisting of —C₁₋₃alkyl, —C₂₋₃alkyl-N(C₁₋₃alkyl)₂, —C₂₋₃alkyl-NHC₁₋₃alkyl, optionally substituted 4- to 7-membered monocyclic heterocycloalkyl, and optionally substituted 7- to 12-membered bicyclic heterocycloalkyl, which monocyclic or bicyclic heterocycloalkyl are optionally substituted with any suitable substituent, such as —C₁₋₃alkyl;
R^{L} is —C₁₋₃alkyl or a 4-7 membered heterocycloalkyl, which heterocycloalkyl is optionally substituted with C₁₋₃alkyl;
wherein when R^{C} is Y—R^{G}, R^{B} and R^{D} are each independently selected from —H, —OH, halogen, —NH₂, —CN, —C₁₋₃alkyl, —C₁₋₃haloalkyl, —O—C₁₋₃alkyl, —O—C₁₋₃haloalkyl and —C₁₋₃alkyl-O—C₁₋₃alkyl; wherein at least one of the substituents R^{A} to R^{E} is not hydrogen;
   and
A² is selected from formulae 1b or 1c:

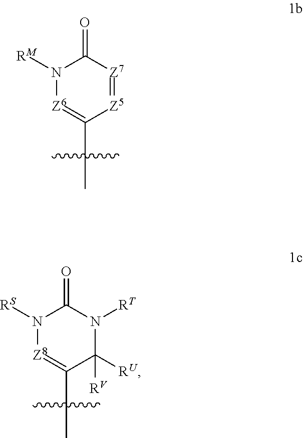

wherein the wavy lines intersect the bond between A² and the carbon atom positioned ortho to R^{A} and R^{E};
R^{M} is selected from the group consisting of optionally substituted C₁₋₆alkyl, optionally substituted C₂₋₆alkenyl, optionally substituted C₁₋₆heteroalkyl, optionally substituted C₃₋₁₀carbocyclyl, C₂₋₆alkynyl and H;
Z⁵ is N or CR^{O};
Z⁶ is N or CR^{P};
Z⁷ is N or CR^{N};
wherein only one of Z⁵, Z⁶ and Z⁷ is N;
Z⁸ is CR^{W} or N;
R^{N} is selected from the group consisting of halogen, optionally substituted —C₁₋₆alkyl, —H, C(O)C₁₋₅alkyl, —NH₂, optionally substituted amino, —OH, cyano, optionally substituted C₁₋₆heteroalkyl, optionally substituted C₃₋₁₀ carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl and thiol;
R^{O} is selected from the group consisting of H, halogen, cyano, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl, hydroxy, thiol and optionally substituted amino;
R^{P} is selected from the group consisting of H, halogen, optionally substituted C₁₋₆alkyl, optionally substituted C₁₋₆heteroalkyl, optionally substituted C₃₋₁₀carbocyclyl and optionally substituted C₆₋₁₀aryl;
alternatively, R^{N} and Z⁵ taken together, combine to form an optionally substituted C₆₋₁₀arene or optionally substituted C₂₋₉heteroarene; optionally wherein R^{N} and R^{O} taken together with the carbon atoms to which they are joined, combine to form an optionally substituted C₆₋₁₀arene or optionally substituted C₂₋₉heteroarene;
R^{S} is selected from the group consisting of H, optionally substituted C₁₋₆alkyl, optionally substituted C₁₋₆heteroalkyl and optionally substituted C₃₋₁₀carbocyclyl;
R^{T} is selected from the group consisting of H, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl, optionally substituted sulfone and optionally substituted sulfonamide, or R^{T} and R^{U} together with the atoms to which each is attached, form an optionally substituted C₂₋₉heterocyclyl;
R^{U} and R^{V} are each independently selected from the group consisting of H, halogen, hydroxyl, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl, thiol, optionally substituted sulfone and optionally substituted amino;
alternatively, R^{T} and R^{U} together with the atoms to which each is attached, form an optionally substituted C₂₋₉heterocyclyl;
R^{W} is selected from the group consisting of H, halogen, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl and optionally substituted C₂₋₉heteroaryl;
and
wherein the BRD9 binder is attached to the linker at any suitable position.

In some examples of formula 1a above, the R^{C} group may be H and the linker may be attached at this position. In other words, the linker (L) may replace the R^{C} group. Such examples may be designated as formula 1a″.

In some examples, the bifunctional molecule is not:

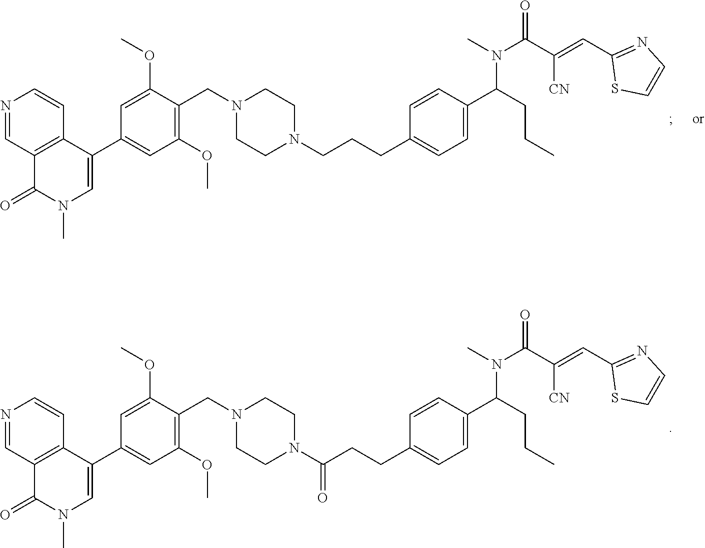 [SMILES: CCCC(c1ccc(CCCN2CCN(Cc3c(OC)cc(-c4cn(C)c(=O)c5cnccc45)cc3OC)CC2)cc1)N(C)C(=O)/C(C#N)=C/c1nccs1 ; CCCC(c1ccc(CCC(=O)N2CCN(Cc3c(OC)cc(-c4cn(C)c(=O)c5cnccc45)cc3OC)CC2)cc1)N(C)C(=O)/C(C#N)=C/c1nccs1]

### Target Protein Binding Ligand (TBL)

As used herein, a “target protein binding ligand” refers to a ligand or moiety, which binds BRD9, e.g. specifically binds BRD9. A bifunctional molecule according to this disclosure may comprise a target protein binding ligand, which binds to the BRD9 target protein with sufficient binding affinity such that the BRD9 target protein is more susceptible to degradation or proteolysis than if unbound by the bifunctional molecule.

A target protein binding ligand may comprise or be derived from a small molecule (or analogue or fragment thereof) already known to act as a modulator, promoter and/or inhibitor of BRD9 protein function. By way of example, the target protein binding ligand may comprise or be derived from a small molecule that is known to inhibit activity of BRD9 target protein.

By way of example, the bifunctional molecules disclosed herein may comprise a target protein binding ligand that binds to BRD9 with sufficient binding affinity such that BRD9 is selectively degraded. In particular, if the bifunctional molecules as described herein were to be contacted with BRD9, the observed DC₅₀ values (for degradation of BRD9) may be less than or equal to about 15 μM, less than or equal to about 10 μM, less than or equal to 1000 nM, less than or equal to 500 nM, less than or equal to 100 nM, or less than or equal to 25 nM, less than or equal to 10 nM, less than or equal to 5 nM, less than or equal to 1.25 nM, less than or equal to 1 nM, or less than or equal to 0.5 nM.

By way of further example, the target protein binding ligand that binds (e.g. specifically binds) to BRD9 may bind to BRD9 with a dissociation constant of less than or equal to about 10 μM, less than or equal to about 5 μM, or less than or equal to about 3 μM. In some examples, the target protein binding ligand that binds (e.g. specifically binds) to BRD9 may bind to BRD9 with a dissociation constant of less than or equal to 1000 nM, less than or equal to 500 nM, less than or equal to 100 nM, less than or equal to 50 nM, or less than or equal to 20 nM. In some examples, the ligand may bind to BRD9 with a dissociation constant of about 0.001 nM to about 10 μM, such as about 0.001 nM to about 8 μM, about 0.001 nM to about 5 μM, about 0.001 nM to about 3 μM or about 0.001 nM to about 2.7 μM. In some examples, the ligand may bind to BRD9 with a dissociation constant of about 0.01 nM to about 10 μM, such as about 0.01 nM to about 8 μM, about 0.01 nM to about 5 μM, about 0.01 nM to about 3 μM or about 0.01 nM to about 2.7 μM.

In some examples, the ligand may bind to BRD9 with a dissociation constant of about 0.1 nM to about 10 μM, such as about 0.1 nM to about 8 μM, about 0.1 nM to about 5 μM, about 0.1 nM to about 3 μM or about 0.1 nM to about 2.7 μM. In some examples, the ligand may bind to BRD9 with a dissociation constant of about 1 nM to about 10 μM, such as about 1 nM to about 8 μM, about 1 nM to about 5 μM, about 1 nM to about 3 μM or about 1 nM to about 2.7 μM.

For the avoidance of doubt, the dissociation constant is a measure of the propensity of an object comprising two components bound together to separate (dissociate) into the two components.

As used herein, the dissociation constant is the measure of the propensity of the complex formed when the target protein binding ligand binds to the target protein to dissociate into separate components, i.e. the propensity of the target protein binding ligand to dissociate from the target protein.

The binding between the BRD9 protein and the target protein binding ligand may comprise one or more binding interactions, such as one or more of the group consisting of hydrogen bonding, dipole-dipole bonding, ion-dipole bonding, ion-induced dipole bonding, ionic bonding and covalent bonding. For example, the binding between the BRD9 protein and the target protein binding ligand may comprise a salt bridge (a combination of hydrogen and ionic bonding).

In some examples, the bifunctional molecules of the disclosure may be selective degraders of BRD9 proteins, for example the bifunctional molecules may selectively degrade BRD9 over other proteins, such as other BRD proteins (e.g. BRD7 or BRD4). In more specific examples, the bifunctional molecules may be selective degraders of certain types of BRD9 protein. By way of example, the molecules of the disclosure may have a greater binding affinity for certain BRD9 mutants than for other types of protein, such as other types of BRD9 protein (e.g. wild type BRD9).

Representative examples of BRD9 targeting agents have been developed over the years, including those described in: WO 2014/114721, WO 2016/077375, WO 2016/077378, WO 2016/139361, WO 2019/152440, a paper by Martin L J. et. al., (Journal of Medicinal Chemistry 2016, 59, 4462-4475) titled “Structure-Based Design of an in Vivo Active Selective BRD9 Inhibitor”; a paper by Theodoulou N. H. et. al., (Journal of Medicinal Chemistry 2015, 59, 1425-1439) titled “Discovery of I-BRD9, a selective Cell Active Chemical Probe for Bromodomain Containing Protein 9 Inhibition”; and a paper by Clack P. et al., (Angewandte Chemie, 2015, 127, 6315-6319).

Such BRD9 binding molecules (as referenced in the paragraph above) can be incorporated into the bifunctional molecules of the present disclosure as the target protein binding ligand (TBL).

As described above, the BRD9 binder of the present disclosure is of formula 1a:

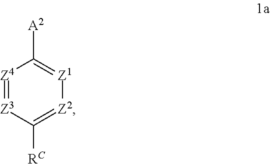

wherein A², Z¹, Z², Z³, Z⁴ and R^{C} are as defined above.

In some embodiments, no more than 1 of Z¹, Z², Z³ and Z⁴ of formula 1a is N. Sometimes, Z¹ is CR^{A}, Z² is CR^{B}, Z³ is N or CR^{D} and Z⁴ is CR^{E}, i.e. only Z³ may be N. In such embodiments, the BRD9 binder may be of formula 1a′:

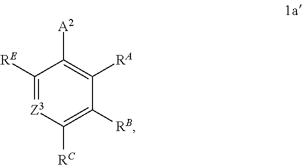

wherein:
R^{A}, R^{B}, R^{C}, R^{E}, Z³ and A² are as defined above and herein.

A² is selected from formulae 1b or 1c:

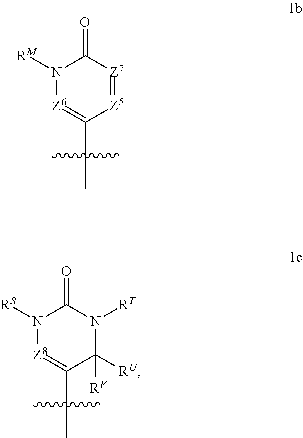

wherein the wavy lines intersect the bond between A² and the carbon atom positioned ortho to R^{A} and R^{E}, and Z⁵, Z⁶, Z⁷, Z⁸, R^{M}, R^{S}, R^{T}, R^{U} and R^{V} are as defined above and herein.

Z⁷ is N or CR^{N} and Z⁵ is N or CR^{O}. In some examples, R^{N} (with the carbon to which it is bonded) and Z⁵ taken together, may combine to form an optionally substituted C₆₋₁₀arene or optionally substituted C₂₋₉heteroarene. For the avoidance of doubt, where Z⁵ is N and R^{N} (with the carbon to which it is bonded) and Z⁵ taken together combine to form an optionally substituted C₆₋₁₀arene or optionally substituted C₂₋₉heteroarene, R^{N} (with the carbon to which it is bonded) and Z⁵ taken together combine to form an optionally substituted N—C₂₋₄heteroarene. For example, where Z⁵ is N, R^{N} and N may combine to form an optionally substituted N—C₂₋₄heteroaryl, as shown below:

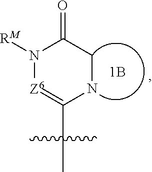

wherein the wavy lines intersect the bond between A² and the carbon atom positioned ortho to R^{A} and R^{E}, Z⁶ and R^{M} are as defined above, and where 1B is an optionally substituted N—C₂₋₄heteroarene, such as an optionally substituted 5 membered heteroarene e.g. any one selected from the optionally substituted group consisting of pyrrole, imidazole, pyrazole and triazole (including 1,2,3 and 1,2,4-triazoles).

In some examples, where Z⁵ is CR^{O} and Z⁷ is CR^{N}, R^{N} and R^{O} taken together with the carbons to which they are bonded, may combine to form an optionally substituted C₆₋₁₀arene or optionally substituted C₂₋₉heteroarene, as shown below:

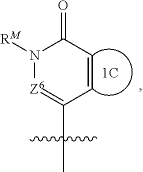

wherein the wavy lines intersect the bond between A² and the carbon atom positioned ortho to R^{A} and R^{E}, Z⁶ and R^{M} are as defined above, and where, as stated above, ring 1C is an optionally substituted C₆₋₁₀arene or optionally substituted C₂₋₉heteroarene. For example, ring 1C may be an optionally substituted benzene or 5-6 membered heteroarene, such as any one selected from the optionally substituted group consisting of benzene, pyridine, pyrrole, imidazole, pyrimidine, thiophene and pyrazole.

In some embodiments, R^{N} (taken with the carbon atoms to which it is joined) and Z⁵ taken together may form a benzene ring or a 5-6 membered heteroarene ring (e.g. ring 1C may be a benzene ring or a 5-6 membered heteroarene), each of which rings can be optionally and independently substituted with one or more groups selected from halogen, —OH, —NH₂, —NH—C₁₋₃alkyl and —C₁₋₅alkyl, C₁₋₅haloalkyl, C₁₋₅alkoxy, C₁₋₄haloalkoxy, 1d, C₃₋₅azacycloalkyl, C₂₋₅alkenyl, C₂₋₅alkynyl, C₃₋₅cycloalkyl, wherein the —C₁₋₅alkyl group can be optionally substituted with 5-6 membered heteroaryl or phenyl; wherein 1d is:

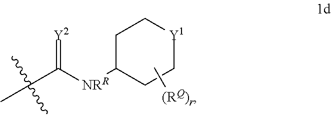

wherein
Y² is NR^{R} or O;
Y¹ is S(O)ₐ or NR^{R};
each R^{R} is independently H or C₁₋₄alkyl;
each R^{Q} is independently selected from the group consisting of C₁₋₄alkyl, C₁₋₄haloalkyl, halogen and —C(O)C₁₋₃alkyl;
a is 0 to 2; and
r is 0 to 3.

In some embodiments, Z⁷ is CR^{N}, i.e. A² is selected from formula 1b′:

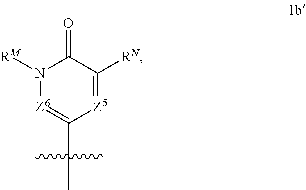

wherein the wavy line intersects the bond between A² and the carbon atom positioned ortho to R^{A} and R^{E}, and Z⁵, Z⁶, R^{M} and R^{N} are as defined above and herein.

As stated previously, R^{M} may be selected from the group consisting of optionally substituted C₁₋₆alkyl, optionally substituted C₂₋₆alkenyl, optionally substituted C₁₋₆heteroalkyl, optionally substituted C₃₋₁₀carbocyclyl, C₂₋₆alkynyl and H. In some embodiments, R^{M} may be selected from the group consisting of optionally substituted C₁₋₆alkyl, optionally substituted C₃₋₆cycloalkyl and H. For example, R^{M} may be selected from the group consisting of C₁₋₆alkyl, C₃₋₆cycloalkyl, C₁₋₆haloalkyl and H. In some embodiments, R^{M} is selected from the group consisting of —C₁₋₅alkyl, -cyclopropyl, —C₁₋₄haloalkyl and H, such as C₁₋₅alkyl. In some embodiments, R^{M} is C₁₋₃alkyl.

As stated previously, R^{N} may be selected from the group consisting of halogen, optionally substituted —C₁₋₆alkyl, —H, C(O)C₁₋₅alkyl, —NH₂, optionally substituted amino, —OH, cyano, optionally substituted C₁₋₆heteroalkyl, optionally substituted C₃₋₁₀ carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl and thiol. In some embodiments, R^{N} may be selected from the group consisting of halogen, optionally substituted C₁₋₆alkyl, H, C(O)C₁₋₅alkyl, —NH₂, —NHC₁₋₃alkyl and —OH. In some embodiments, R^{N} is selected from the group consisting of halogen, —C₁₋₅alkyl, —C₁₋₃haloalkyl, —H, C(O)C₁₋₅alkyl, —NH₂, —NHC₁₋₃alkyl and —OH. For example, R^{N} may be C₁₋₅alkyl or halogen.

As described above, Z⁵ is N or CR^{O}, where R^{O} is selected from the group consisting of H, halogen, cyano, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl, hydroxy, thiol and optionally substituted amino. For example, R^{O} may be H or optionally substituted C₁₋₆alkyl, such as C₁₋₃alkyl. In some embodiments, R^{O} may be H or —C₁₋₃alkyl.

In some embodiments, R^{N} is —C₁₋₅alkyl or halogen, or R^{N} and Z⁵ taken together form an optionally substituted 5-6 membered heteroarene or benzene ring. In some embodiments, the optionally substituted 5-6 membered heteroarene ring may comprise one or more heteroatoms selected from the group consisting of N, S and O, such as N and S, i.e. the optionally substituted 5-6 membered heteroarene ring may be an N- or S-heteroarene. In some embodiments, the optionally substituted 5-6 membered heteroarene ring is any one selected from the optionally substituted group consisting of pyridine, pyrrole, imidazole, pyrimidine, thiophene and pyrazole.

For the avoidance of doubt, the optional substituents may be one or more groups selected from halogen, —OH, —NH₂, —NH—C₁₋₃alkyl and —C₁₋₅alkyl, C₁₋₅haloalkyl, C₁₋₅alkoxy, C₁₋₄haloalkoxy, 1d, C₃₋₅azacycloalkyl, C₂₋₅alkenyl, C₂₋₅alkynyl, C₃₋₅cycloalkyl, wherein the —C₁₋₅alkyl group can be optionally substituted with 5-8 membered heteroaryl or phenyl; wherein 1d is:

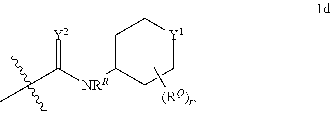

wherein
Y² is NR^{R} or O;
Y¹ is S(O)ₐ or NR^{R};
each R^{R} is independently H or C₁₋₄alkyl;
each R^{Q} is independently selected from the group consisting of C₁₋₄alkyl, C₁₋₄haloalkyl, halogen and —C(O)C₁₋₃alkyl;
a is 0 to 2; and
r is 0 to 3.

For example, the optional substituents may be independently selected from the group consisting of halogen, —OH, —NH₂, —NH—C₁₋₃alkyl —C₁₋₅alkyl, C₁₋₅haloalkyl, C₁₋₅alkoxy and C₁₋₄haloalkoxy. In some cases, the optional substituents may be independently selected from C₁-C₄alkyl, allyl, crotyl, C₂₋₅alkenyl, C₂₋₅alkynyl, C₁₋₅haloalkyl, C₃₋₅cycloalkyl, C₁-C₄alkoxy, and halo. In some embodiments, where R^{N} and Z⁵ taken together combine to form an optionally substituted C₆₋₁₀aryl or optionally substituted C₂₋₉heteroaryl, the C₆₋₁₀aryl or C₂₋₉heteroaryl is not substituted.

As described above, Z⁶ is N or CR^{P}, where R^{P} is selected from the group consisting of H, halogen, optionally substituted C₁₋₆alkyl, optionally substituted C₁₋₆heteroalkyl, optionally substituted C₃₋₁₀carbocyclyl and optionally substituted C₆₋₁₀aryl. For example, R^{P} may be H or optionally substituted C₁₋₆alkyl, such as H or C₁₋₆alkyl. In some embodiments, R^{P} is H or —C₁₋₃alkyl, i.e. Z⁶ is N, CH or C—C₁₋₃alkyl. For example, Ze may be CH or C—C₁₋₃alkyl.

In some particular embodiments, A² is selected from formula 1b′, wherein formula 1b′ is:

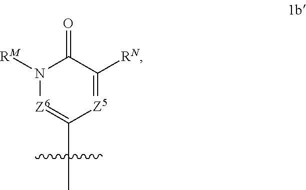

wherein the wavy line intersects the bond between A² and the carbon atom positioned ortho to R^{A} and R^{E};
R^{M} is selected from the group consisting of —C₁₋₅alkyl, -cyclopropyl, —C₁₋₄haloalkyl and H;
R^{N} is selected from the group consisting of halogen, —C₁₋₅alkyl, —C₁₋₃haloalkyl, —H, C(O)C₁₋₅alkyl, —NH₂, —NHC₁₋₃alkyl and —OH;
Z⁵ is N or CR^{O}
Z⁶ is N or CR^{P} wherein only one of Z⁵ and Z⁶ may be N;
R^{O} is H or —C₁₋₃alkyl;
R^{P} is H or —C₁₋₃alkyl;
wherein only one of R^{O} and R^{P} may be —C₁₋₃alkyl;
alternatively, R^{N} and Z⁵ taken together form a benzene ring or a 5-6 membered heteroarene ring, each of which rings can be optionally and independently substituted with one or more groups selected from halogen, —OH, —NH₂, —NH—C₁₋₃alkyl and —C₁₋₅alkyl, C₁₋₅haloalkyl, C₁₋₅alkoxy, C₁₋₄haloalkoxy, 1d, C₃₋₅azacycloalkyl, C₂₋₅alkenyl, C₂₋₅alkynyl, C₃₋₅cycloalkyl, wherein the —C₁₋₅alkyl group can be optionally substituted with 5-6 membered heteroaryl or phenyl;
wherein 1d is:

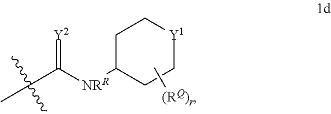

wherein
Y² is NR^{R} or O;
Y¹ is S(O)ₐ or NR^{R};
each R^{R} is independently H or C₁₋₄alkyl;
each R^{Q} is independently selected from the group consisting of C₁₋₄alkyl, C₁₋₄haloalkyl, halogen and —C(O)C₁₋₃alkyl;
a is 0 to 2; and
r is 0 to 3.

As described above, the BRD9 binder is attached to the linker at any suitable position (provided it has the correct valency and/or is chemically suitable). For example, the linker may be attached to the BRD9 binder by way of a covalent bond between an atom on the linker and an atom forming part of R^{C}, R^{A}, R^{B}, R^{D} or R^{E}. Alternatively, the linker may be attached directly to the ring to which R^{C}, R^{A}, R^{B}, R^{D} and/or R^{E} are bound, i.e. the linker may replace R^{C}, R^{A}, R^{B}, R^{D} or R^{E}. In some embodiments, the linker is attached to the BRD9 binder by way of a covalent bond between an atom on the linker and an atom forming part of R^{C} or by way of a covalent bond between an atom on the linker and the atom to which R^{C} would otherwise be bound, i.e. the linker replaces R^{C}.

Alternatively, where R^{C} and Z² or R^{C} and Z³ taken together (e.g. R^{C} and R^{B} or R^{C} and R^{D} taken together with the carbon atoms to which they are joined) form a 5-7 membered heterocycloalkyl optionally substituted with —C₁₋₃alkyl, the linker may be attached to the BRD9 binder by way of a covalent bond between an atom on the linker and an atom forming part of the 5-7 membered heterocycloalkyl.

In some embodiments, the BRD9 binder is of formula 1a¹, 1a², 1a³:

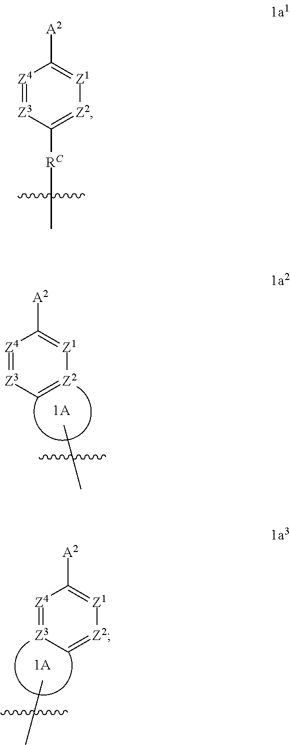

wherein the wavy line intersects the bond between the BRD9 binder and the linker;
A², Z¹, Z², Z³ and Z⁴ are as defined above and herein;
R^{C} is absent or is as defined above and herein;
and ring 1A is a 5-7 membered heterocycloalkane optionally substituted with —C₁₋₃alkyl.

Ring 1A may comprise one or two heteroatoms independently selected from the list consisting of N, S and O. For example, ring 1A may be selected from the list consisting of pyrrolidine, piperidine, piperazine, morpholine, oxolane, oxane, tetrahydrothiophene and thiane. In some cases, ring 1A may be an N-heterocycloalkane such as pyrrolidine, piperdine or piperazine. In particular examples, ring 1A is pyrrolidine.

For the avoidance of doubt, where the linker is attached to the BRD9 binder by way of a covalent bond between an atom on the linker and an atom forming part of a feature on the BRD9 binder (such as R^{C}), the linker replaces a chemical group or an atom of the feature with a valency of 1 (such as a hydrogen atom) in order for valencies to be satisfied. For example, where the feature on the BRD9 binder is dimethylamido (—C(O)N(CH₃)₂) or dimethylaminomethylene (—CH₂N(CH₃)₂), the linker may replace a methyl group or a hydrogen atom on the feature.

As another alternative, the linker may be attached to the BRD9 binder by way of a covalent bond between an atom on the linker and an atom forming part of A², for example an atom forming part of R^{M}, R^{N}, R^{O}, R^{P}, R^{S}, R^{T}, R^{U}, R^{V}, or R^{W} or the linker may replace R^{M}, R^{N}, R^{O}, R^{P}, R^{S}, R^{T}, R^{U}, R^{V}, or R^{W}. Alternatively, where R^{N} and Z⁵ taken together combine to form an optionally substituted C₆₋₁₀arene or optionally substituted C₂₋₉heteroarene; optionally wherein R^{N} and R^{O}taken together with the carbon atoms to which they are joined, combine to form an optionally substituted C₆₋₁₀arene or optionally substituted C₂₋₉heteroarene, the linker may be attached to the BRD9 binder by way of a covalent bond between an atom on the linker and an atom forming part of the optionally substituted C₆₋₁₀arene or optionally substituted C₂₋₉heteroarene.

In one exemplary BRD9 binder, where R^{N} and Z⁵ taken together combine to form an optionally substituted thiophene, the linker may be attached to the BRD9 binder as shown in the structure below:

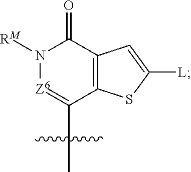

wherein the wavy line intersects the bond between A² and the carbon atom positioned ortho to R^{A} and R^{E}; and R^{M} and Z⁶ are as defined above and herein.

The linker may be attached to an atom forming part of a substituent bonded to the same positions indicated above. For example, the linker may be attached to an atom forming part of substituent 1d bonded to the same positions indicated above. This is exemplified in the structure below, where R^{N} and Z⁵ taken together combine to form an optionally substituted thiophene; the wavy line intersects the bond between A² and the carbon atom positioned ortho to R^{A} and R^{E}; and Y² is O, Y¹ is N, R^{R} is H, and R^{Q} and r are as defined above:

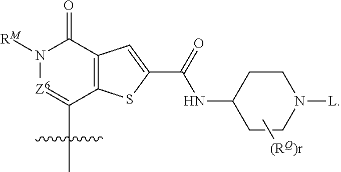

As described above, Z¹, Z², Z³, Z⁴ and R^{C} of the BRD9 binder are defined as follows:
Z¹ is N or CR^{A};
Z² is N or CR^{B};
Z³ is N or CR^{D};
Z⁴ is N or CR^{E};
wherein no more than 3 of Z¹, Z², Z³ and Z⁴ are N;
R^{A} and R^{E} are each independently selected from the group consisting of —H, —O—C₁₋₃alkyl and —C₁₋₃alkyl;
R^{B} and R^{D} are each independently selected from the group consisting of —O—C₁₋₃alkyl, —H, —OH, halogen, —NH₂, —C₁₋₃alkyl, —O—C₁₋₃haloalkyl, —C₁₋₃alkyl-O—C₁₋₃alkyl, 4-7 membered heterocycloalkyl, —C₁₋₃alkyl-SO₂—C₁₋₃alkyl, —C₁₋₃alkyl-NH₂, —C₁₋₃alkyl-N(—C₁₋₃alkyl)₂, —N(C₁₋₃alkyl)₂, —NH—R^{F};
R^{F} is selected from —SO₂—C₁₋₃alkyl and —C₁₋₃alkyl, wherein the —C₁₋₃alkyl is optionally substituted with a 5 to 6 membered heteroaryl;
alternatively, R^{A} and R^{B} taken together form a benzene ring; alternatively, R^{C} and Z² or R^{C} and Z³ taken together (e.g. R^{C} and R^{B} or R^{C} and R^{D} taken together with the carbon atoms to which they are joined) form a 5-7 membered heterocycloalkyl optionally substituted with —C₁₋₃alkyl;
R^{C} is selected from the group consisting of —H, —Y—R^{G}, —NH₂, —C₁₋₃alkyl and 4-7 membered heterocycloalkyl;
Y is absent or is selected from the group consisting of —CR^{H}R^{I}—, —SO₂— and —CO—;
R^{H} and R^{I} are each independently selected from —H or —C₁₋₃alkyl; or R^{H} and R^{I} taken together form a —C₃₋₄cycloalkyl,
R^{G} is selected from the group consisting of —NH₂, —OH, —C₁₋₃alkyl, —N(R^{J}R^{K}), —O—R^{L}, aryl, 5-6 membered heteroaryl, wherein the aryl and heteroaryl are optionally and independently substituted with one or more halogen, optionally substituted 4- to 7-membered monocyclic heterocycloalkyl, and optionally substituted 7- to 12-membered bicyclic heterocycloalkyl, which monocyclic or bicyclic heterocycloalkyl are optionally substituted with any suitable substituent, such as one or more groups independently selected from halogen, —OH, —NH₂, —C₁₋₃alkyl, —NHC₁₋₃alkyl, —N(C₁₋₃alkyl)₂, —O—C₁₋₃alkyl and —CH₂—R^{M1};
R^{M1} is selected from 5-10 membered mono- or bicyclic aryl or heteroaryl, which is optionally substituted with —NH₂, —OH, halogen, —CN, C₁₋₃alkyl, —O—C₁₋₃alkyl;
R^{J} is —H or —C₁₋₃alkyl;
R^{K} is selected from the group consisting of —C₁₋₃alkyl, —C₂₋₃alkyl-N(C₁₋₃alkyl)₂, —C₂₋₃alkyl-NHC₁₋₃alkyl, optionally substituted 4- to 7-membered monocyclic heterocycloalkyl, and optionally substituted 7- to 12-membered bicyclic heterocycloalkyl, which monocyclic or bicyclic heterocycloalkyl are optionally substituted with any suitable substituent, such as —C₁₋₃alkyl;
R^{L} is —C₁₋₃alkyl or a 4-7 membered heterocycloalkyl, which heterocycloalkyl is optionally substituted with C₁₋₃alkyl;
wherein when R^{C} is Y—R^{G}, R^{B} and R^{D} are each independently selected from —H, —OH, halogen, —NH₂, —CN, —C₁₋₃alkyl, —C₁₋₃haloalkyl, —O—C₁₋₃alkyl, —O—C₁₋₃haloalkyl and —C₁₋₃alkyl-O—C₁₋₃alkyl;
wherein at least one of the substituents R^{A} to R^{E} is not hydrogen.

In alternative examples of the above, the list of groups for R^{G} and R^{K} may be replaced as follows:
R^{G} may be selected from the group consisting of —NH₂, —OH, —C₁₋₃alkyl, —N(R^{J}R^{K}), —O—R^{L}, aryl, 5-6 membered heteroaryl, wherein the aryl and heteroaryl are optionally and independently substituted with one or more halogen, 4-7 membered heterocycloalkyl, which heterocycloalkyl is optionally substituted with one or more groups independently selected from halogen, —OH, —NH₂, —C₁₋₃alkyl, —NHC₁₋₃alkyl, —N(C₁₋₃alkyl)₂, —O—C₁₋₃alkyl and —CH₂R^{M1};
R^{K} may be selected from the group consisting of —C₁₋₃alkyl, —C₂₋₃alkyl-N(C₁₋₃alkyl)₂, —C₂₋₃alkyl-NHC₁₋₃alkyl and 4-7 membered heterocycloalkyl, which heterocycloalkyl is optionally substituted with —C₁₋₃alkyl; and
   wherein R^{J}, R^{L}, R^{M1} are as defined above.

In some examples of the BRD9 binding ligands described herein (and unless otherwise stated):
(i) R^{A}, R^{B}, R^{D} and R^{E} are independently selected from the group consisting of —O—C₁₋₃alkyl, —H, halogen, —O—C₁₋₃haloalkyl, —OH, —NH₂, —C₁₋₃alkyl, —C₁₋₃alkyl-NH₂, —C₁₋₃alkyl-N(—C₁₋₃alkyl)₂ and —N(C₁₋₃alkyl)₂; or
(ii) R^{A}, R^{D} and R^{E} are independently selected from the group consisting of —O—C₁₋₃alkyl, —H, halogen, —O—C₁₋₃haloalkyl, —OH, —NH₂, —C₁₋₃alkyl, —C₁₋₃alkyl-NH₂, —C₁₋₃alkyl-N(—C₁₋₃alkyl)₂ and —N(C₁₋₃alkyl)₂ and R^{B} and R^{C} taken together form a 5-7 membered heterocycloalkyl optionally substituted with —C₁₋₃alkyl.

In such embodiments, the 5-7 membered heterocycloalkyl may be as defined above for ring 1A. In some embodiments, R^{A}, R^{B}, R^{D} and R^{E} are independently selected from the group consisting of —O—C₁₋₃alkyl, —H, halogen, —O—C₁₋₃haloalkyl, —OH, —NH₂, —C₁₋₃alkyl, —C₁₋₃alkyl-NH₂, —C₁₋₃alkyl-N(—C₁₋₃alkyl)₂ and —N(C₁₋₃alkyl)₂. For example, R^{A}, R^{B}, R^{D} and R^{E} may be independently selected from the group consisting of —O—C₁₋₃alkyl, —H, halogen and —O—C₁₋₃haloalkyl.

In some cases, at least one of R^{A}, R^{B}, R^{D} and R^{E} may be —H. For example, at least one of R^{A} and R^{B} may be —H. In particular embodiments, at least two of R^{A}, R^{B}, R^{D} and R^{E} are —H.

In some embodiments, at least one of R^{A}, R^{B}, R^{D} and R^{E} is selected from the group consisting of —O—C₁₋₃alkyl, halogen and —O—C₁₋₃haloalkyl. Sometimes, R^{B} and R^{E} are selected from the group consisting of —O—C₁₋₃alkyl, halogen and —O—C₁₋₃haloalkyl.

In some embodiments, R^{C} is —H or —Y—R^{G}. Y may be —C^{H}R^{I}— or —CO—, wherein R^{H} and R^{I} are as defined above. Each of R^{H} and R^{I} may be —H; or R^{H} and R^{I} taken together may form a —C₃₋₄cycloalkyl.

R^{G} may be as defined above, or may be selected from the group consisting of —NH₂, —OH, —C₁₋₃alkyl —N(R^{J}R^{K}), —O—R^{L} and optionally substituted 4- to 7-membered monocyclic heterocycloalkyl, and optionally substituted 7- to 12-membered bicyclic heterocycloalkyl, where R^{J}, R^{K} and R^{L} and the optional substituents of the 4- to 7-membered monocyclic heterocycloalkyl and 7- to 12-membered bicyclic heterocycloalkyl are as defined above. R^{J} may be —H or —C₁₋₃alkyl and R^{K} may be selected from —C₁₋₃alkyl, optionally substituted 4- to 7-membered monocyclic heterocycloalkyl, and optionally substituted 7- to 12-membered bicyclic heterocycloalkyl. R^{L} may be —C₁₋₃alkyl.

Where R^{G} or R^{K} is an optionally substituted 4- to 7-membered monocyclic heterocycloalkyl, the optionally substituted 4- to 7-membered monocyclic heterocycloalkyl may be a 5- to 7-membered monocyclic heterocycloalkyl comprising between one and three ring heteroatoms selected from N, O and S. In some examples, the optionally substituted 4- to 7-membered monocyclic heterocycloalkyl may be a 5- to 7-membered monocyclic heterocycloalkyl comprising one or two ring heteroatoms selected from N. In some examples, the optionally substituted 4- to 7-membered monocyclic heterocycloalkyl may be piperazinyl, piperidinyl or diazepanyl (each of which may optionally comprise between one and three substituents as described herein).

Where R^{G} or R^{K} is an optionally substituted 7- to 12-membered bicyclic heterocycloalkyl, the optionally substituted 7- to 12-membered bicyclic heterocycloalkyl may be a bridged bicyclic ring or a spirocyclic bicyclic ring (i.e it may comprise two rings joined at a spiro centre). By way of example only, the optionally substituted 7- to 12-membered bicyclic heterocycloalkyl may be a bridged piperazinyl or bridged piperidinyl. In other examples, the optionally substituted 7- to 12-membered bicyclic heterocycloalkyl may be an optionally substituted spirocyclic bicyclic heterocycloalkyl comprising between one and three ring heteroatoms selected from N, O and S (e.g. between one and two ring heteroatoms selected from N). In some examples, the optionally substituted 7- to 12-membered bicyclic heterocycloalkyl may be spirocyclic and comprise a first 5- or 6-membered ring and a second 3- to 6-membered ring.

In some examples, R^{C} may be any one selected from:

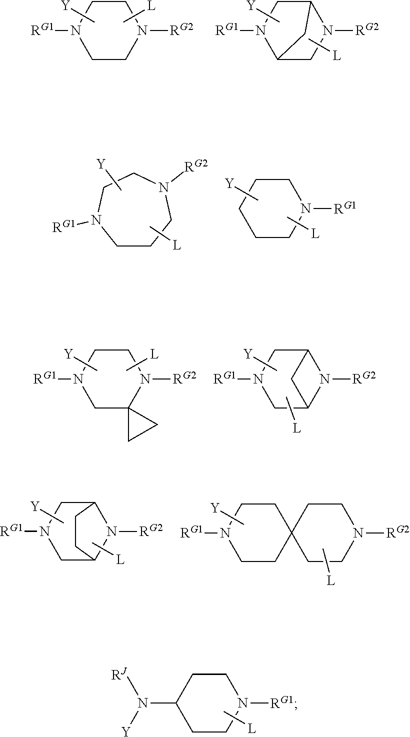

wherein Y is CR^{H}R^{I} (e.g. CH₂);
R^{G1} and R^{G2} are each independently selected from H and C₁-C₃ alkyl;
R^{J} is as defined above and herein; and
L shows the point of attachment of the linker.

In the structures shown above, both the Y and L groups may be attached to the heterocyclic ring(s) by way of a covalent bond between an atom on the Y and L group respectively and an atom on the heterocyclic ring. These groups may be bonded at any chemically suitable position provided valencies are satisfied (e.g. by replacing a H atom).

By way of further example only, R^{C} may be any one selected from:

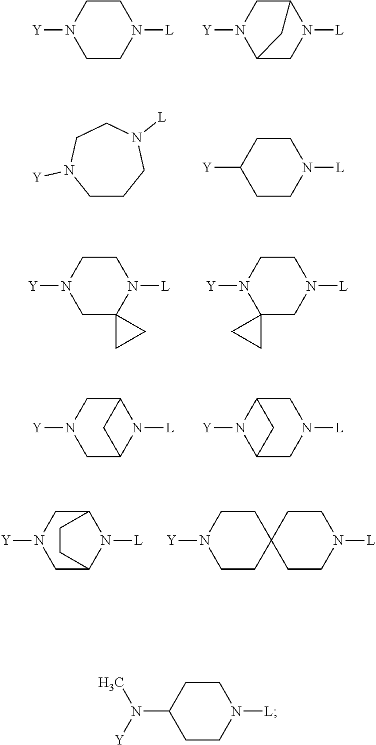

wherein Y is CR^{H}R^{I} (e.g. CH₂); and
L shows the point of attachment of the linker.

In particular embodiments, R^{C} is any one selected from the group consisting of

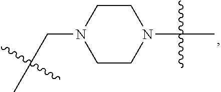 [SMILES: *CN1CCN(*)CC1]

—CH₂N(C₁₋₃alkyl)₂, —C(O)N(C₁₋₃alkyl)₂, —C(CH₂CH₂)N(C₁₋₃alkyl)₂, and CH₂OCH₃, wherein the wavy lines intersect the bond between R^{C} and the rest of the BRD9 binder and the bond between R^{C} and the linker.

In some embodiments, the BRD9 binder is of formula 1e, 1f or 1g:

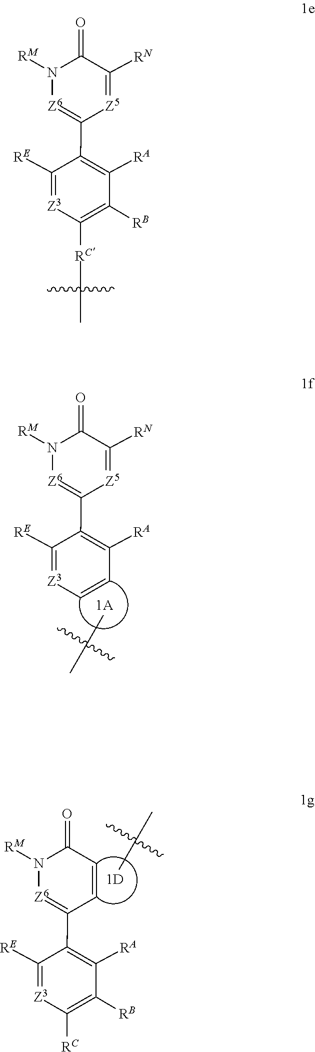

wherein the wavy line intersects the bond between the BRD9 binder and the linker;
R^{A}, R^{B}, R^{E}, R^{M}, R^{N}, Z³, Z⁵ and Z⁶ are as defined above;
R^{C} is absent, or is as defined for R^{C} above and herein;
ring 1A is a 5-7 membered heterocycloalkane optionally substituted with —C₁₋₃alkyl; and
ring 1D is an optionally substituted C₆₋₁₀arene or optionally substituted C₂₋₉heteroarene.

In some embodiments, ring 1D is optionally substituted benzene or an optionally substituted 5-6 membered heteroarene. The 5-6 membered heteroarene may comprise one or more heteroatoms selected from the group consisting of S, N and O, such as S. In some cases, ring 1D may be a 5-6 membered N-heteroarene or S-heteroarene, for example any one selected from the group consisting of thiophene, pyrazole, imidazole, pyrrole, pyrimidine and pyridine. In particular examples, ring 1D is thiophene fused to the rest of the BRD9 binder at the 2′ and 3′ positions and, in even more particular examples, bonded to the linker by way of a covalent bond between an atom on the linker and the carbon atom at the 5′ position of the thiophene. In such particular examples, the BRD9 binder may be of formula 1g′:

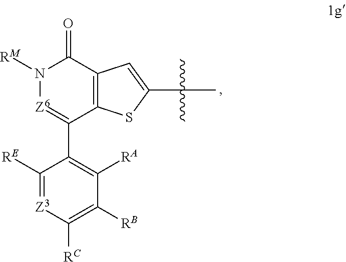

wherein the wavy line intersects the bond between the BRD9 binder and the linker; and wherein R^{A}, R^{B}, R^{C}, R^{E}, R^{M}, Z³, and Z⁶ are as defined above.

In some embodiments, ring 1A is pyrrolidine. In particular examples, ring 1A is pyrrolidine fused to the rest of the BRD9 binder at the 3′ and 4′ positions and, in even more particular embodiments, bonded to the linker by way of a covalent bond between an atom on the linker and the nitrogen atom of the pyrrolidine. In such particular embodiments, the BRD9 binder may be of formula 1f′:

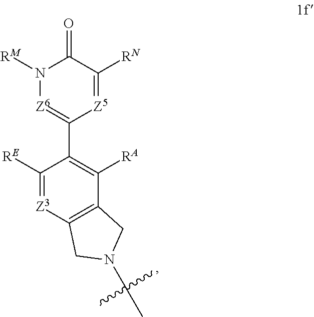

wherein the wavy line intersects the bond between the BRD9 binder and the linker; and wherein R^{A}, R^{E}, R^{M}, R^{N}, Z³, Z⁵ and Z⁶ are as defined above and herein.

In some embodiments, the BRD9 binder is of formula 1e, 1f′ or 1g′.

In particular embodiments, the BRD9 binder is any one of formulae 1ea to 1eh, 1fa to 1fh and 1ga:

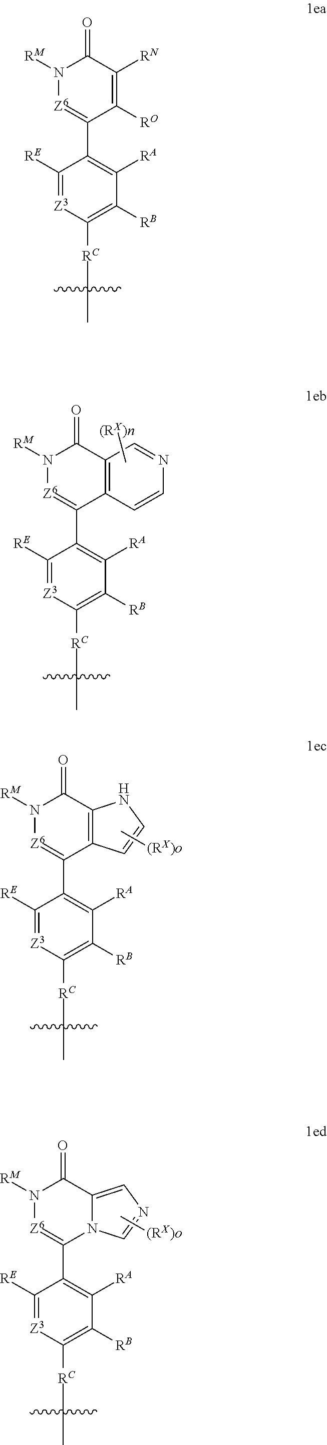

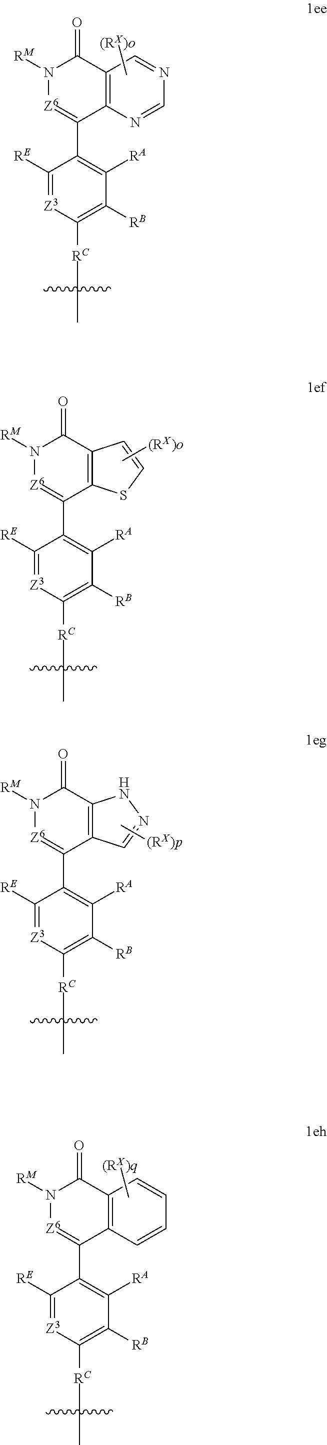

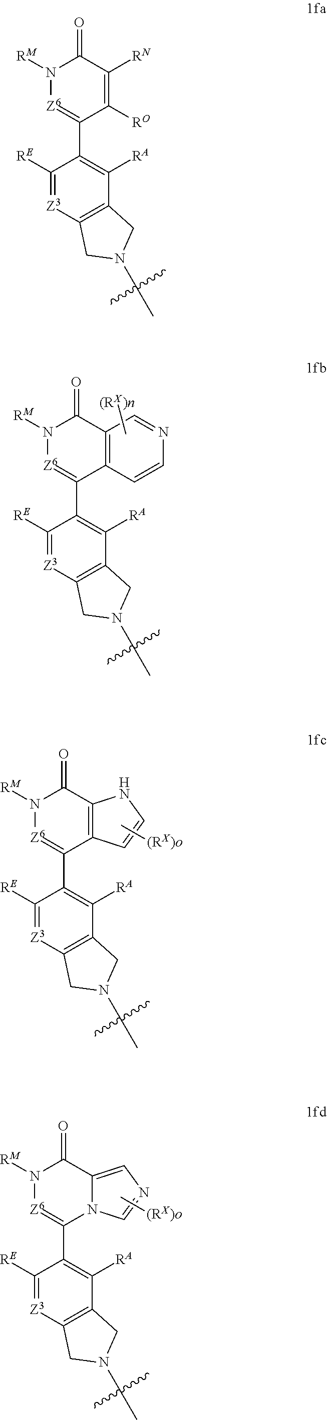

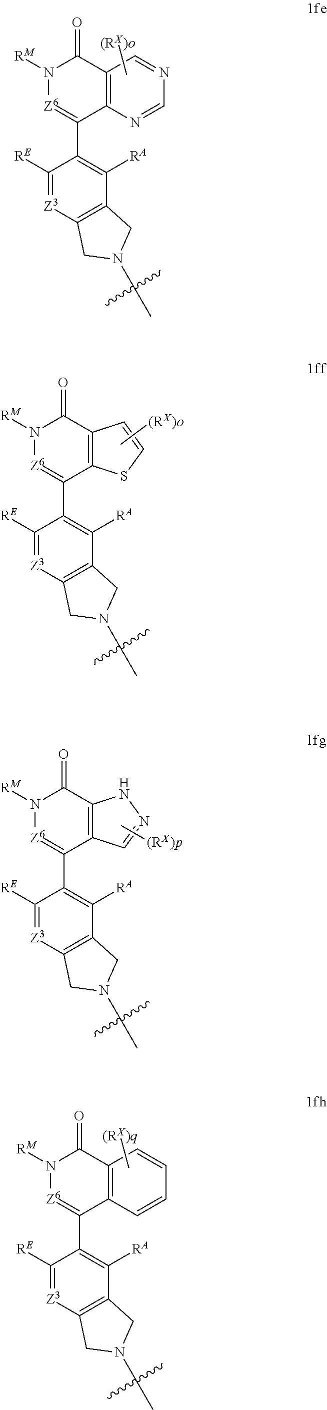

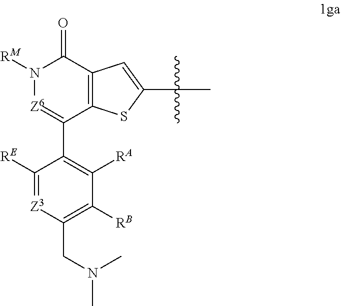

wherein the wavy line intersects the bond between the BRD9 binder and the linker;
R^{A}, R^{B}, R^{E}, R^{M}, Z³ and Z⁶ are as defined above and herein;
R^{C} is absent, or is as defined above and herein;
R^{N} is as defined above and herein, for example is selected from the group consisting of halogen, —C₁₋₅alkyl, —C₁₋₃haloalkyl, —H, C(O)C₁₋₅alkyl, —NH₂, —NHC₁₋₃alkyl and —OH;
R^{O} is as defined above and herein, for example is —H or —C₁₋₃alkyl;
each R^{X} is as defined for the optional substituents of the optionally substituted C₆₋₁₀aryl or optionally substituted C₂₋₉heteroaryl formed from R^{N} and Z⁵ (taken together), for example each R^{X} may be independently selected from the group consisting of halogen, —OH, —NH₂, —NH—C₁₋₃alkyl —C₁₋₅alkyl, C₁₋₅haloalkyl, C₁₋₅alkoxy and C₁₋₄haloalkoxy;
n is 0 to 3 (such as 0);
o is 0 to 2 (such as 0);
p is 0 or 1 (such as 0); and
q is 0 to 4 (such as 0).

Each of n, o, p and q may be 0.

In some embodiments, the BRD9 binder is according to formula 1ea′:

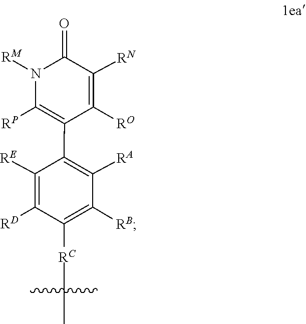

wherein the wavy line intersects the bond between the BRD9 binder and the linker;
R^{A} and R^{E}are as defined above and herein, for example are each independently selected from H and —O—C₁₋₃alkyl;
R^{B} and R^{D} are as defined above and herein, for example are each independently selected from —O—C₁₋₃alkyl, —H, -halo, —C₁₋₃alkyl, and —O—C₁₋₃haloalkyl;
R^{C} is absent, or is —Y—R^{G};
Y is selected from the group consisting of —CR^{H}R^{I}—, and —CO—;
R^{H} and R^{I} are each independently selected from —H or —C₁₋₃alkyl; or R^{H} and R^{I} taken together form a —C₃₋₄cycloalkyl;
R^{G} is selected from the group consisting of —N(R^{J}R^{K}) (e.g. —N(C₁₋₃alkyl)-, —N(C₁₋₃alkyl)(optionally substituted 4- to 7-membered monocyclic heterocycloalkylene), or —N(C₁₋₃alkyl)(optionally substituted 7- to 12-membered bicyclic heterocycloalkylene)); —C—; optionally substituted 4- to 7-membered monocyclic heterocycloalkylene; and optionally substituted 7- to 12-membered bicyclic heterocycloalkylene;
R^{J} and R^{K} are as defined above and herein;
R^{M} is as defined above and herein, for example is C₁₋₃alkyl; and
R^{N}, R^{O} and R^{P} are each as defined above and herein, for example are each independently selected from the group consisting of halo, —C₁₋₃alkyl, and —C₁₋₃haloalkyl.

In even more particular embodiments, the BRD9 binder is any one of formulae 1h to 1z and 2a to 2g:

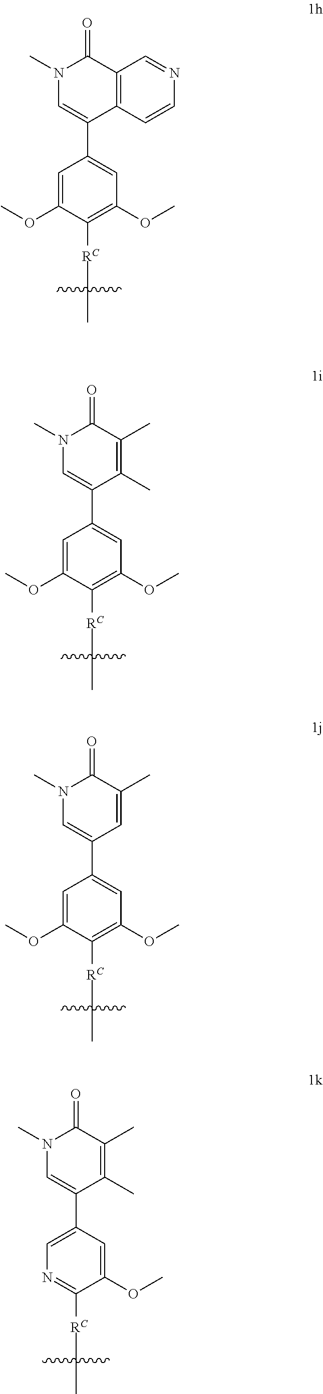

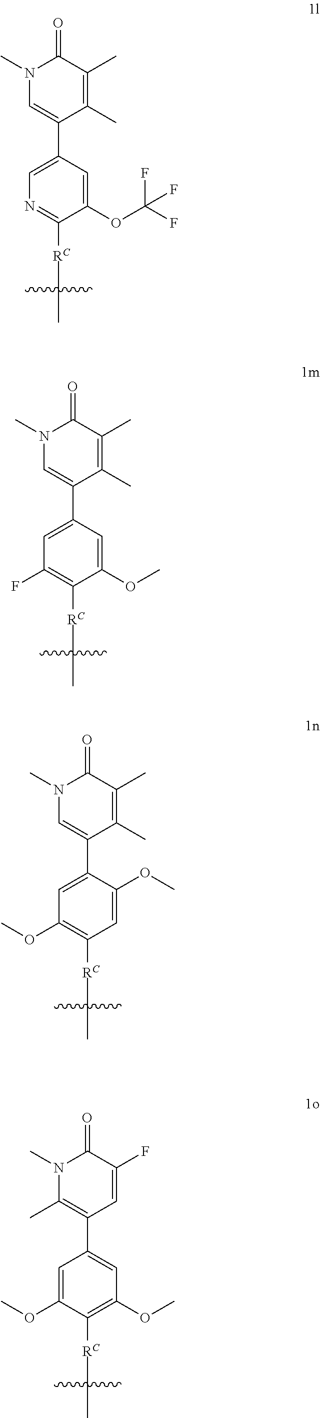

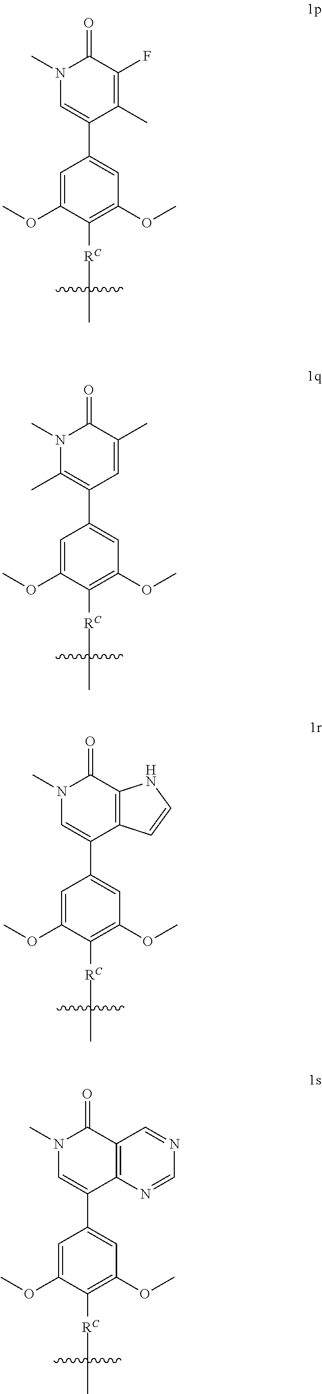

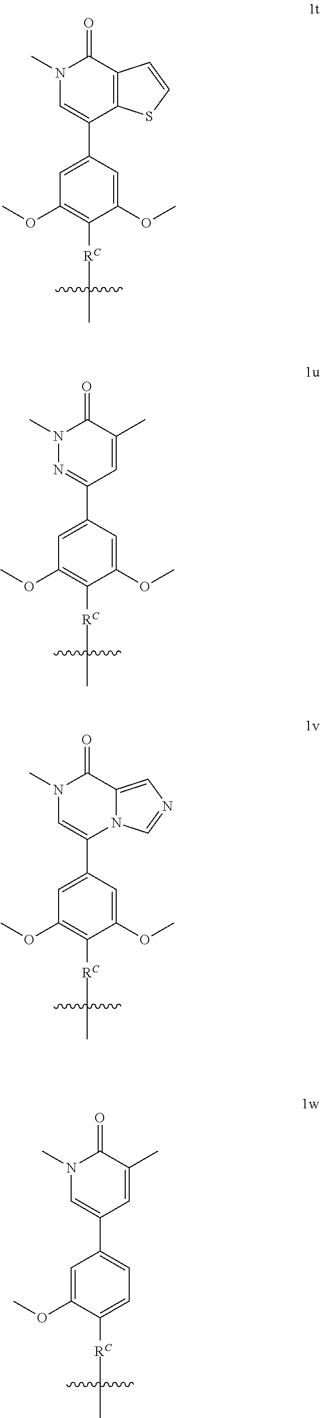

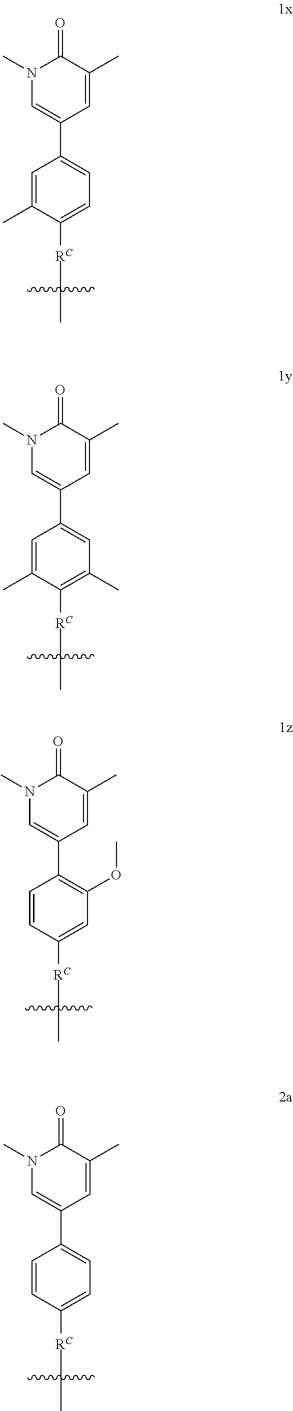

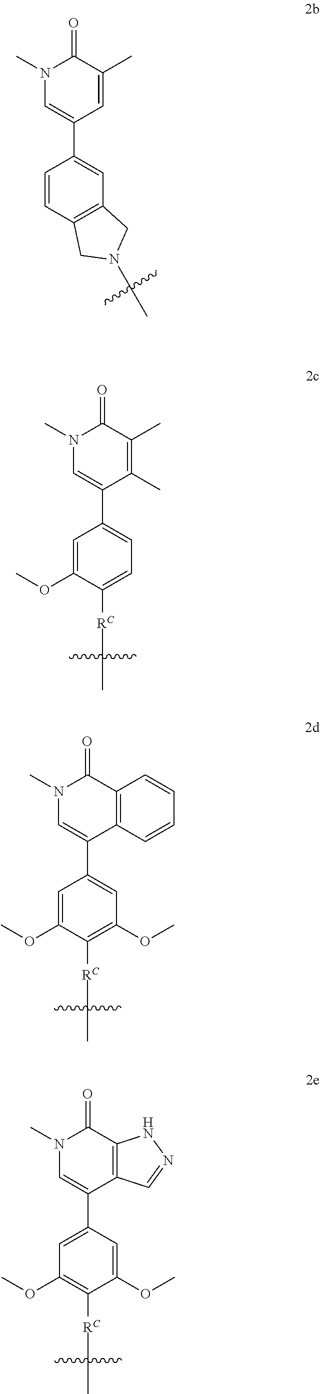

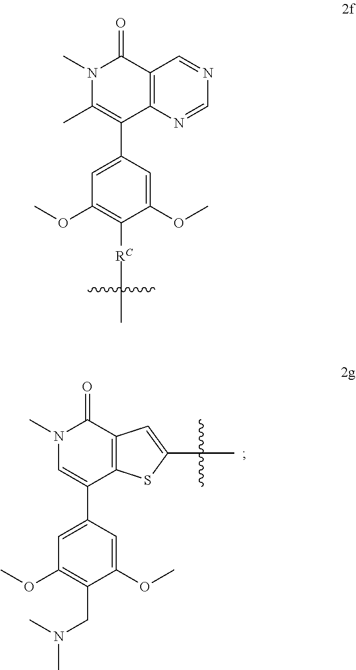

wherein R^{C} is absent, or is —Y—R^{G};
Y is selected from the group consisting of —CR^{H}R^{I}—, and —CO—;
R^{H} and R^{I} are each —H; or R^{H} and R^{I} taken together form a —C₃₋₄cycloalkyl;
R^{G} is selected from the group consisting of —N(R^{J}R^{K}) (e.g. —N(C₁₋₃alkyl)-, N(C₁₋₃alkyl)(optionally substituted 4- to 7-membered monocyclic heterocycloalkylene), or —N(C₁₋₃alkyl)(optionally substituted 7- to 12-membered bicyclic heterocycloalkylene)); —C—; optionally substituted 4- to 7-membered monocyclic heterocycloalkylene containing one or two N ring atoms; and optionally substituted 7- to 12-membered bicyclic heterocycloalkylene containing one or two N ring atoms;
R^{J} and R^{K} are as defined above and herein;
wherein the wavy line intersects the bond between the BRD9 binder and the linker.

In particular examples of any of the above formulae (e.g. any one of formulae 1e, 1g, 1g′, 1ea to 1eh, 1ea′, 1h to 1z and 2a to 2g, and unless otherwise stated), R^{G} is —N(C₁₋₃alkyl)-, —O— or

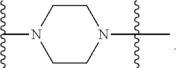 [SMILES: *N1CCN(*)CC1]

In some examples of any of the above formulae (e.g. any one of formulae 1e, 1g, 1g′, 1ea to 1eh, 1ea′, 1h to 1z and 2a to 2g, and unless otherwise stated), R^{C} may be any one selected from:

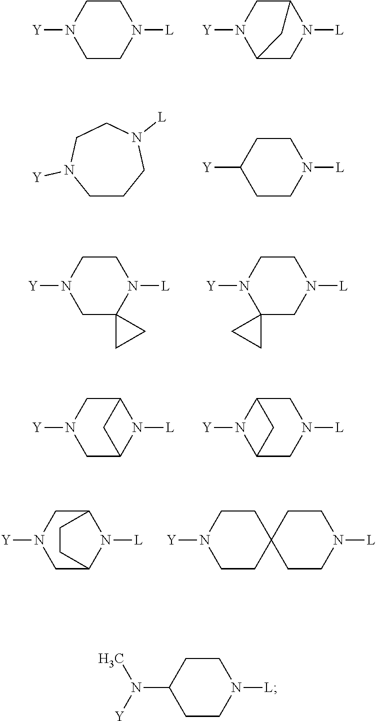

wherein Y is CR^{H}R^{I} (e.g. CH₂) or —CO—;
R^{H} and R^{I} are as defined above and herein; and
L shows the point of attachment of the linker.

In particular, in each of the structures shown above, Y may be CH₂.

In some examples of any of the above formula (e.g. any one of formulae 1e, 1g, 1g′, 1ea to 1eh, 1ea′, 1h to 1z and 2a to 2g, and unless otherwise stated), R^{C} may be absent and the linker may be attached (i.e. covalently bonded) to the parent structure at this position. Such examples may be designated with ″ and so be referred to as formulae 1e″, 1g″, 1g′″, 1ea″ to 1eh″, 1ea″, 1h″ to 1z″ and 2a″ to 2g″ respectively herein.

In some embodiments, the BRD9 binder is any one of formulae 1 h, 1i, 1j, 1m, 1t, 2c or 2e:

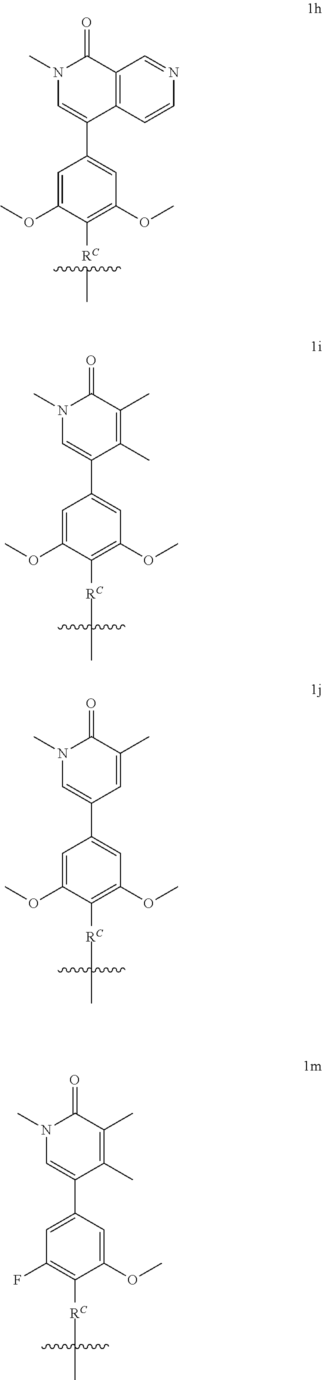

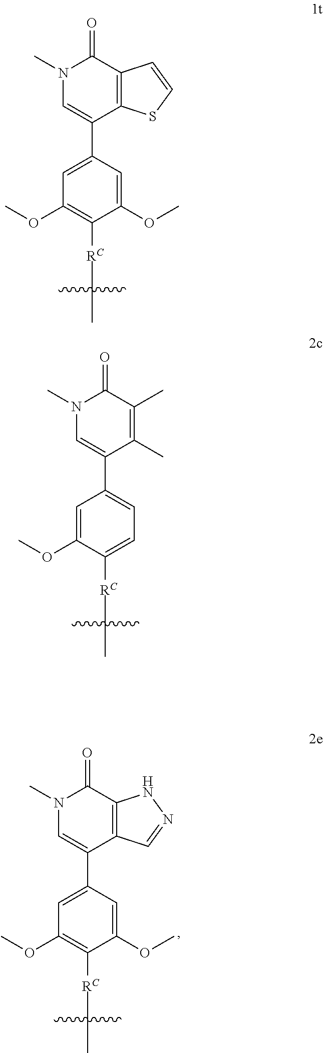

wherein R^{C} is absent, or is —Y—R^{G};
Y is selected from the group consisting of —CR^{H}R^{I}—, and —CO—;
R^{H} and R^{I} are each —H; or R^{H} and R^{I} taken together form a —C₃₋₄cycloalkyl;
R^{G} is selected from the group consisting of —N(R^{J}R^{K}) (e.g. —N(C₁₋₃alkyl)-, N(C₁₋₃alkyl)(optionally substituted 4- to 7-membered monocyclic heterocycloalkylene), or —N(C₁₋₃alkyl)(optionally substituted 7- to 12-membered bicyclic heterocycloalkylene)); —O—; optionally substituted 4- to 7-membered monocyclic heterocycloalkylene containing one or two N ring atoms; and optionally substituted 7- to 12-membered bicyclic heterocycloalkylene containing one or two N ring atoms;
R^{J} and R^{K} are as defined above and herein;
wherein the wavy line intersects the bond between the BRD9 binder and the linker.

In particular examples of any of the above formulae, R^{G} is —N(C₁₋₃alkyl)-, —O— or

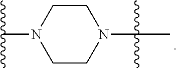 [SMILES: *N1CCN(*)CC1]

In some examples of any of the above formulae, R^{C} may be any one selected from:

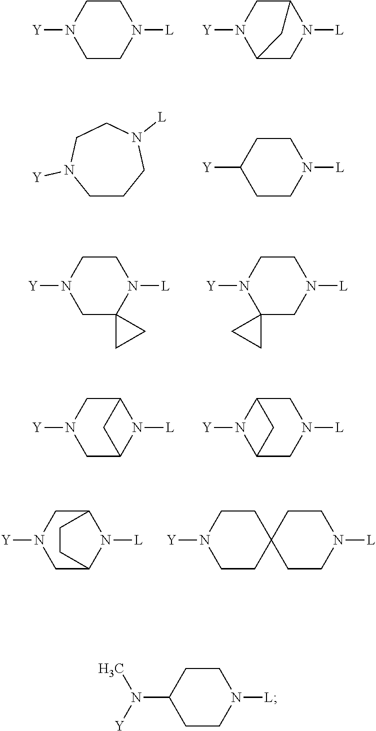

wherein Y is CR^{H}R^{I} (e.g. CH₂) or —CO—;
R^{H} and R^{I} are as defined above and herein; and
L shows the point of attachment of the linker.

In particular, in each of the structures shown above, Y may be CH₂.

In some examples of any of the above formula, R^{C} may be absent and the linker may be attached (i.e. covalently bonded) to the parent structure at this position. Such examples may be designated with ″ and so be referred to as formulae 1h″, 1i″, 1j″, 1m″, 1t″, 2c″ or 2e″ respectively herein.

In some embodiments, the BRD9 binder is selected from the following:

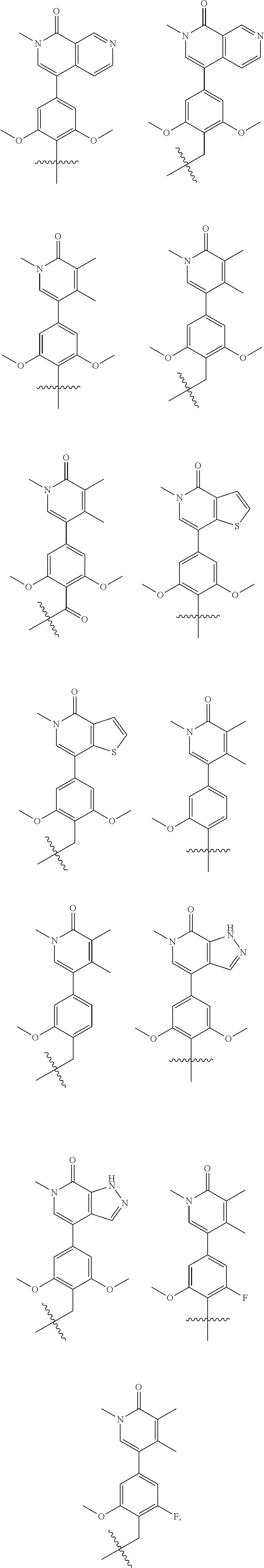

wherein the wavy line intersects the bond between the BRD9 binder and the linker.

In some cases, the BRD9 binder may not be:

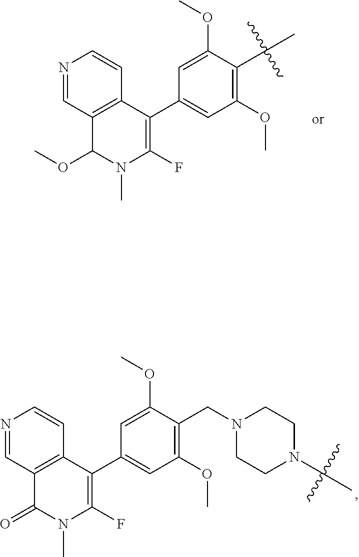

wherein the wavy line intersects the bond between the BRD9 binder and the linker.

### Warhead (Z)

Z comprises a structure according to formula (I) or formula (WI).

As shown in formulae (I) and (WI), a double bond is present in Z. The stereochemistry of this double bond may be either E or Z and this is indicated by the wavy line bond in formula (I) and (WI) (and is similarly shown on the other formulae and structures disclosed herein). The designation of this moiety as either E or Z may depend on the identity of the R³ or R^{3A} group. In some examples, Z may comprise a mixture of E and Z stereoisomers. Thus, the present disclosure includes within its scope the use of each individual E and Z stereoisomers of any of the disclosed Z moieties according to formulae (I) and (WI) and any of the other formulae described herein (e.g. in a substantially stereopure form), as well as the use of mixtures of these E and Z isomers. In some cases, the stereochemistry of the double bond and the moieties bound to it is Z, i.e. the Z stereoisomer. In other examples, the stereochemistry of the double bond and the moieties bound to it is E, i.e. the E stereoisomer.

For the avoidance of doubt, where the double bond of Z of formula (I) or (WI) is shown in a structure herein to be a specific stereoisomer (E or Z) in any of the specific examples of this disclosure, it need not be in that specific stereoisomer. In other words, both E and Z stereoisomers and mixtures of the two are included within the scope of the structure irrespective of the specific stereoisomer shown.

As stated above, in some examples, formula (I) is:

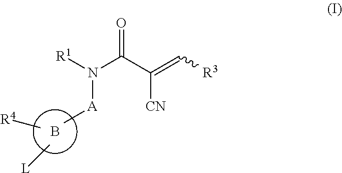

wherein R¹, R³, R⁴, A, B and L are as defined above.

On ring B, groups R⁴ and A may be held at adjacent positions on the aryl, heteroaryl, substituted aryl or substituted heteroaryl ring. In other words, the R⁴ and A groups may be in a 1,2 substitution pattern with one another, or may be separated by 3 bonds. For the avoidance of doubt, where B is a heteroaryl or substituted heteroaryl, a heteroatom contained within ring B may be directly bonded to A or R⁴.

As shown in formula (I) above, the linker is appended to moiety Z via ring B. The linker may be attached to moiety Z by way of a covalent bond between an atom on the linker and an atom contained in the ring system of the optionally substituted aryl or heteroaryl group of ring B. This linker may be attached to ring B at any position on the optionally substituted aromatic or heteroaromatic ring (provided it has the correct valency and/or is chemically suitable). For example, the linker may replace a hydrogen atom at any position on the aromatic or heteroaromatic ring.

In other examples, Z may comprise a structure as shown in formula (I) above, wherein:
A, B, X and R⁴ are as defined above; and wherein
R¹ is selected from optionally substituted C₁ to C₆ alkyl, optionally substituted C₁ to C₆ haloalkyl, optionally substituted benzyl, optionally substituted carbocyclyl, and optionally substituted heterocycyl;
R² and R^{2′} are each independently selected from H and optionally substituted C₁ to C₆ alkyl, or wherein R² and R^{2′} together form a 3-, 4-, 5- or 6-membered optionally substituted carbocyclic or heterocyclic ring; and
R³ is selected from optionally substituted C₁ to C₆ alkyl, optionally substituted aryl, optionally substituted heteroaryl, optionally substituted carbocyclyl and optionally substituted heterocyclyl.

In those cases where R¹ and R⁴ together form an optionally substituted 5-, 6-, or 7-membered heterocyclic ring, Z may be represented by formula (Ia):

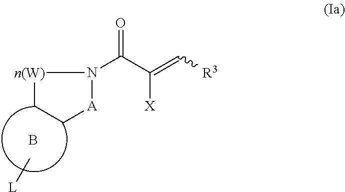

wherein A, B, R³ and L are as defined for formula (I); and
n is 1, 2 or 3;
W is selected from CR^{W1}R^{W2}, O, NR^{W3}, and S; and
R^{W1}, R^{W2} and R^{W3} are each independently selected from H and C₁ to C₆ alkyl; and
wherein when n is 2 or 3, each W is independently selected from CR^{W1}R^{W2}, O, NR^{W3}, and S.

In those cases, where R¹ and R² together form an optionally substituted 5-, 6-, or 7-membered heterocyclic ring, Z may be represented as formula (Ib):

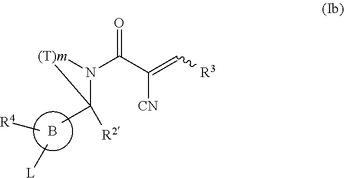

Wherein B, R², R³, R⁴ and L are as defined for formula (I);
m is 3, 4 or 5;
each T is independently selected from CR^{T1}R^{T2}, O, NR^{T3}, and S; and
R^{T1}, R^{T2} and R^{T3} are each independently selected from H and C₁ to C₆ alkyl.

In those cases where R² and R⁴ together form an optionally substituted 5-, 6-, or 7-membered heterocyclic or carbocyclic ring, Z may be represented as formula (Ic):

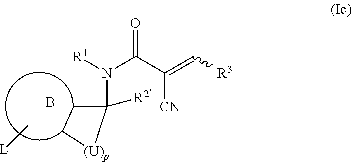

Wherein B, R¹, R^{2′}, R³ and L are as defined for formula (I);
p is 2, 3 or 4; and
each U is independently selected from CR^{U1}R^{U2}, O, NR^{U3}, and S; and
R^{U1}, R^{U2} and R^{U3} are each independently selected from H and C₁ to C₆ alkyl.

With respect to the various structures for Z defined by the formulae herein, R¹ may be C₁ to C₆ alkyl, such as C₁ to C₄ alkyl. For example, R¹ may be selected from the group consisting of methyl, ethyl, n-propyl, iso-propyl.

As stated above for formula (I), A is either absent or is CR²R^{2′}. In some cases, where A is CR²R^{2′}, R² and R^{2′} are each independently selected from H and C₁ to C₆ alkyl, optionally wherein the C₁ to C₆ alkyl is substituted with one or more halo atoms (such as F, Cl, or Br). In further examples where A is CR²R^{2′}, R² and R^{2′} are each independently selected from H and C₁ to C₆ alkyl, such as methyl, ethyl, n-propyl, iso-propyl and n-butyl. In some examples, one of R² and R^{2′} is a hydrogen and the other is C₁ to C₆ alkyl. For example, R² may be methyl, ethyl, n-propyl or iso-propyl and R^{2′} may be H. In other examples, both R² and R^{2′} are each independently selected from C₁ to C₆ alkyl (e.g. both R² and R^{2′} may be methyl). In some examples, R² and R^{2′} are each independently selected from H and C₁ to C₃ alkyl substituted with one or more halo atoms (such as trifluromethyl).

As stated above, R³ is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S. In some examples, R³ is selected from C₁ to C₆ alkyl, carbocyclyl, substituted carbocyclyl, heterocyclyl and substituted heterocyclyl, optionally wherein the C₁ to C₆ alkyl is substituted with one or more heteroatoms selected from halo, N, O and S and/or is substituted with a carbocyclic or heterocyclic group. For example, R³ may be selected from heteroaryl, substituted heteroaryl, substituted C₁-C₆ alkyl, substituted C₃-C₆ cycloalkyl, substituted C₃-C₆ heterocycloalkyl, C₁-C₆ alkyl substituted with a heterocyclic group, aryl, and substituted aryl.

Representative examples of suitable R³ groups include, but are not limited to, thiazolyl, pyridinyl, benzothiazolyl, phenyl, pyrazolyl, isoxazolyl, isothiazolyl, tetrahydropyranyl, oxetanyl, cyclobutanyl, cyclopropanyl, tert-butyl, imidazolyl, oxazolyl, thiophenyl, imidazo(1,2-a)pyridinyl, N—C₁ to C₆ alkylenemorpholine, and 4,5,6,7-tetrahydro-1,3-benzothiazolyl, such as thiazolyl, pyridinyl, benzothiazolyl, phenyl, pyrazolyl, isoxazolyl, isothiazolyl, tetrahydropyranyl, oxetanyl, cyclobutanyl, cyclopropanyl and tert-butyl.

In each case, these R³ groups may be substituted, such as substituted thiazolyl, substituted pyridinyl, substituted benzothiazolyl, substituted phenyl, substituted pyrazolyl, substituted isoxazolyl, substituted isothiazolyl, substituted tetrahydropyranyl, substituted oxetanyl, substituted cyclobutanyl, substituted cyclopropanyl and substituted tert-butyl. Where R³ is a substituted heteroaryl or aryl group, there may be one or more substituents on the aromatic ring e.g. it may be mono-, di- or tri-substituted. Where R³ is optionally substituted pyrazolyl or imidazolyl, a nitrogen atom of the pyrazolyl or imidazolyl ring may be substituted with C₁ to C₆ alkyl, such as methyl.

Examples of suitable R³ groups are shown below:

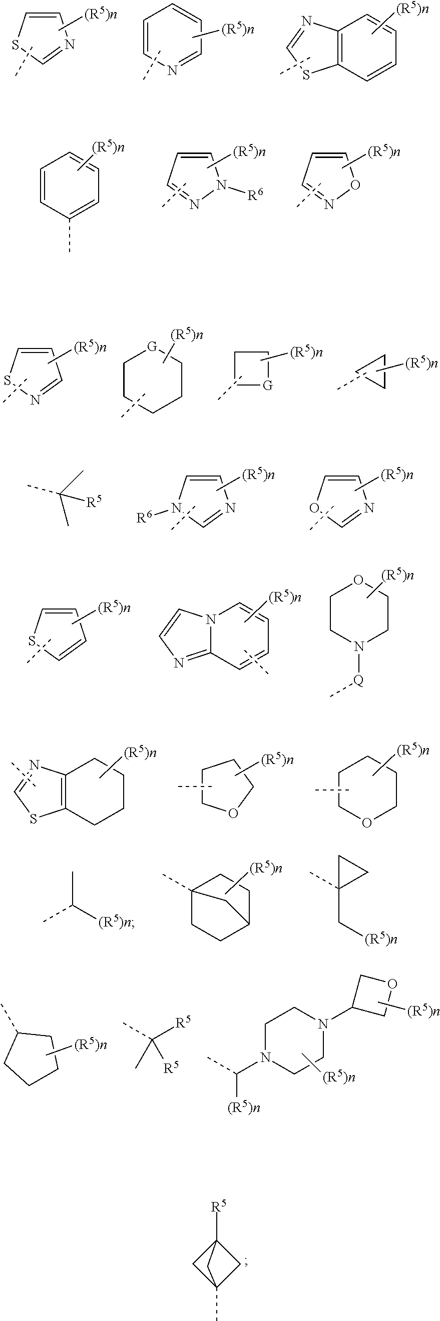

wherein the dotted line on the structures indicates the position that each of the respective R³ groups may be joined to the structure shown in formulae described herein. Where the dotted line is not shown connected directly to an atom, the R³ group may be connected to the structure shown in the formulae by a covalent bond to an atom at any position on the aromatic ring (provided that it has the correct valency and/or is chemically suitable). For example, a hydrogen at any position on the R³ group may be replaced with a bond to the parent structures shown in formulae described herein.

R⁵ may be any substituent as described herein or may be absent. In some examples, R⁵ may be selected from halo (e.g. F, Cl, Br, I), CF₃, —CH₂F, —OCF₃, —OCH₂F, —OCHF₂, —CHF₂, C₁ to C₆ alkyl, —CN, —OH, —OMe, —SMe, —SOMe, —SO₂Me, —NH₂, —NHMe, —NMe₂, CO₂Me, —NO₂, CHO, and COMe.

As stated above, there may be one or more substituents on the aromatic ring (e.g. n may be 0 to 5, such as 0 to 4, 0 to 3, or 0 to 2). Where more than one substituent is present, each substituent may be independently selected from the R⁵ groups noted above.

R⁶ may be C₁ to C₆ alkyl, such as methyl.

G may be selected from CH₂, O and NH.

Q may be C₁ to C₆ alkylene such as dimethylmethylene (—C(CH₃)₂—) or dimethylethylene (—C(CH₃)₂CH₂—).

Further examples of suitable R³ groups are shown below:

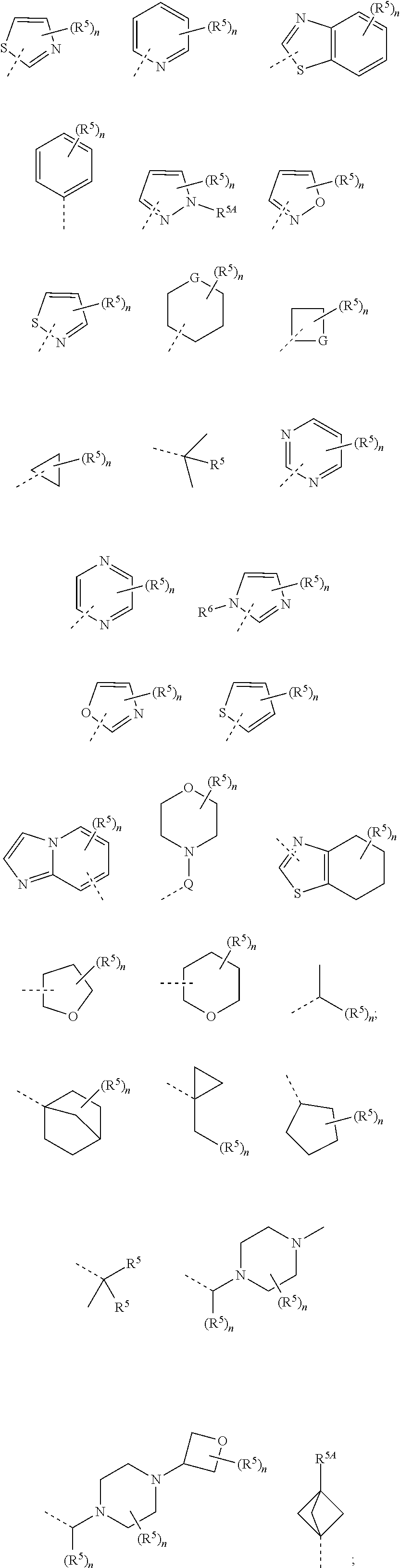

wherein the dotted line on the structures indicates the position that each of the respective R³ groups may be joined to the structure shown in formulae described herein. Where the dotted line is not shown connected directly to an atom, the R³ group may be connected to the structure shown in the formulae by a covalent bond to an atom at any position on the aromatic ring (provided that it has the correct valency and/or is chemically suitable). For example, a hydrogen at any position on the R³ group may be replaced with a bond to the parent structures shown in formulae described herein.

R⁵ may be any substituent as described herein or may be absent. In some examples, R⁵ may be selected from halo (e.g. F, Cl, Br, I), CH₂OH, CF₃, —CH₂F, —OCF₃, —OCH₂F, —OCHF₂, —CHF₂, C₁ to C₆ alkyl, —CN, —OH, —OMe, —SMe, —SOMe, —SO₂Me, —NH₂, —NHMe, —NMe₂, CO₂Me, —NO₂, CHO, and COMe. As stated above, there may be one or more substituents on the aromatic ring (e.g. n may be 0 to 5, such as 0 to 4, 0 to 3, or 0 to 2). Where more than one substituent is present, each substituent may be independently selected from the R⁵ groups noted above.

R⁶ may be C₁ to C₆ alkyl, such as methyl.

G may be selected from CH₂, O and NH.

Q may be C₁ to C₆ alkylene such as dimethylmethylene (—C(CH₃)₂—) or dimethylethylene (—C(CH₃)₂CH₂—).

In further embodiments, R³ is selected from the group consisting of

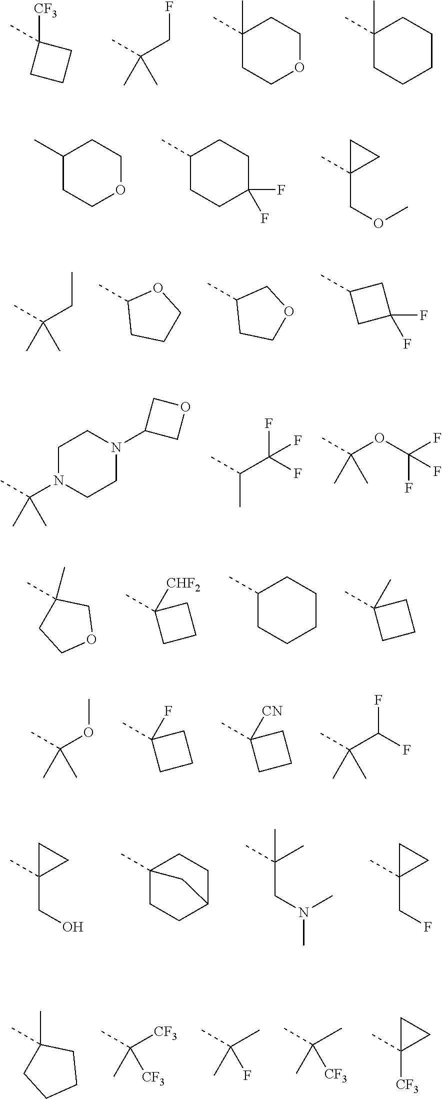

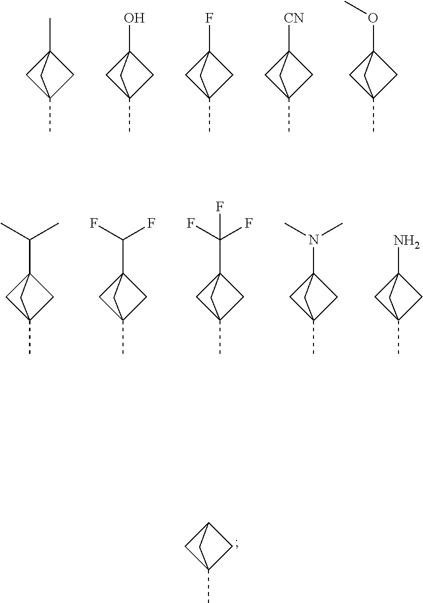

wherein the dotted line indicates the position at which each of the respective R³ groups is joined to the structure in the formulae described herein.

By way of further example, R⁵ may be selected from C₁ to C₆ alkyl (e.g. methyl) and halo (e.g. F).

As stated above, there may be one or more substituents on the aromatic ring. Where two or more substituents are present, each substituent may be independently selected from the R⁵ groups noted above. Again, where present and unless otherwise indicated, R⁵ may be appended to the aryl or heteroaryl ring at any position (provided that it has the correct valency and/or is chemically suitable).

By way of further example, a suitable R³ group may be selected from the following:

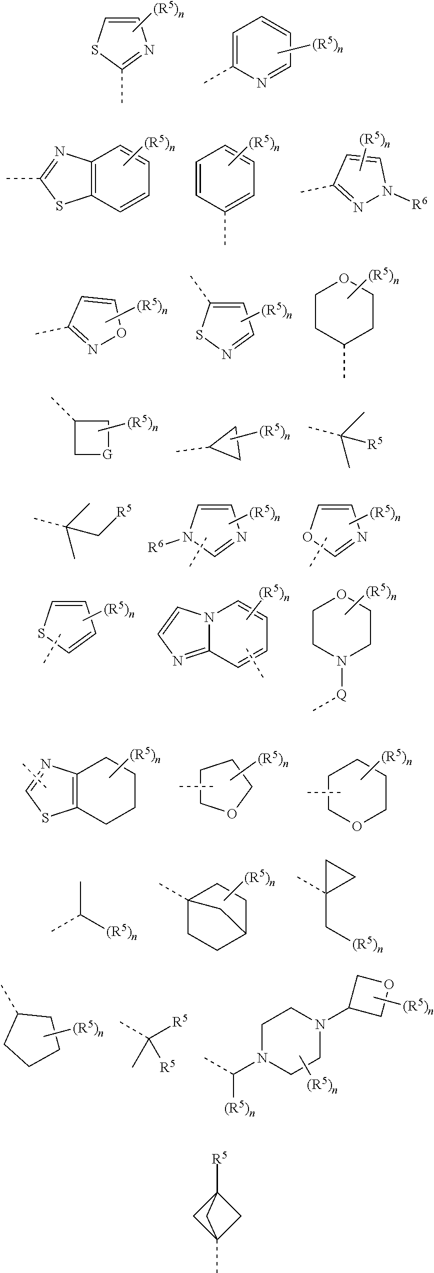

wherein the dotted line on the structures indicates the position that each of the respective R³ groups may be joined to the structure shown in formulae (I) to (Ic), and R⁵, R⁶, n and G are as defined above.

Further examples of suitable R³ groups are shown below:

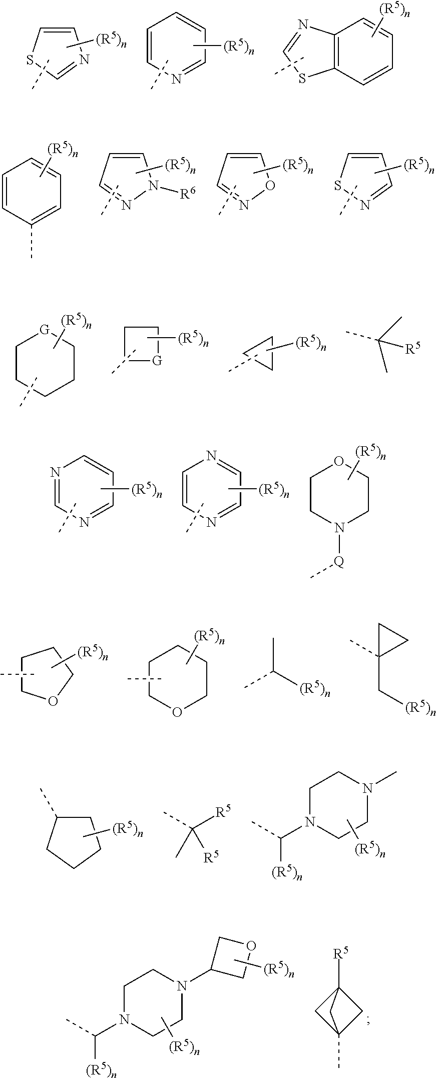

wherein the dotted line on the structures indicates the position that each of the respective R³ groups may be joined to the structure shown in formulae described herein. Where the dotted line is not shown connected directly to an atom, the R³ group may be connected to the structure shown in the formulae by a covalent bond to an atom at any position on the aromatic ring (provided that it has the correct valency and/or is chemically suitable). For example, a hydrogen at any position on the R³ group may be replaced with a bond to the parent structures shown in formulae described herein.

R⁵ may be any substituent as described herein or may be absent. In some examples, R⁵ may be selected from halo (e.g. F, Cl, Br, I), CH₂OH, CF₃, —CH₂F, —OCF₃, —OCH₂F, —OCHF₂, —CHF₂, C₁ to C₆ alkyl, —CN, —OH, —OMe, —SMe, —SOMe, —SO₂Me, —NH₂, —NHMe, —NMe₂, CO₂Me, —NO₂, CHO, and COMe. As stated above, there may be one or more substituents on the aromatic ring (e.g. n may be 0 to 5, such as 0 to 4, 0 to 3, or 0 to 2). Where more than one substituent is present, each substituent may be independently selected from the R⁵ groups noted above.

R⁶ may be C₁ to C₆ alkyl, such as methyl.

G may be selected from CH₂, O and NH.

Q may be C₁ to C₆ alkylene such as dimethylmethylene (—C(CH₃)₂—) or dimethylethylene (—C(CH₃)₂CH₂—).

By way of further example, a suitable R³ group may be selected from the following:

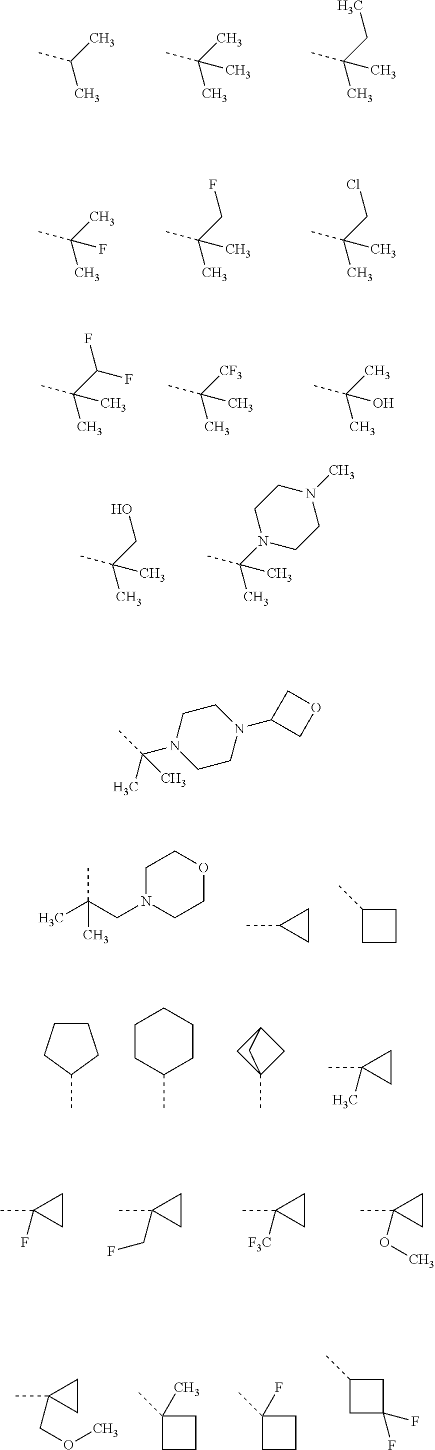

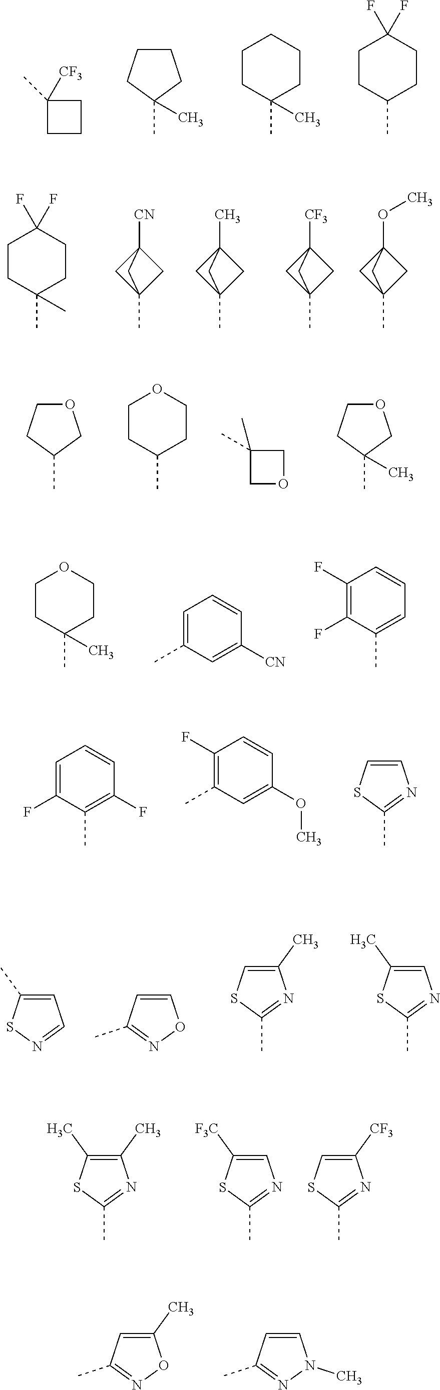

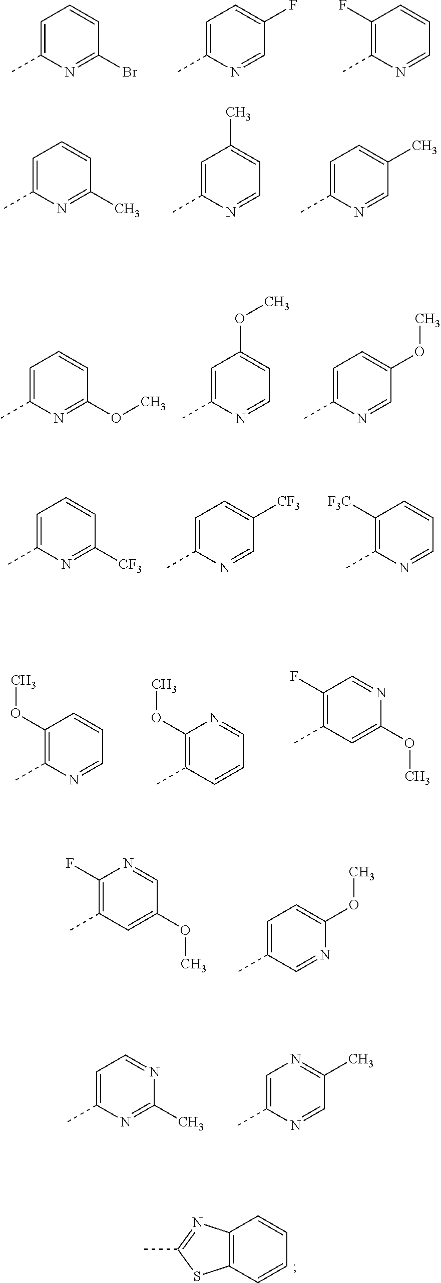

wherein the dotted line on the structures indicates the position that each of the respective R³ groups may be joined to the structure shown in formulae (I) to (Ic).

In certain examples, Z comprises a structure according to formula (II):

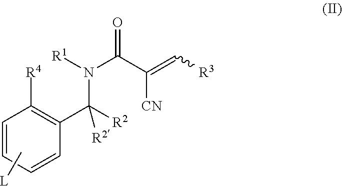

wherein
R¹ is selected from C₁ to C₆ alkyl, benzyl, substituted benzyl, carbocyclyl, substituted carbocyclyl, heterocyclyl and substituted heterocyclyl, optionally wherein the C₁ to C₆ alkyl is substituted with one or more heteroatoms selected from halo, N, O and S and/or is substituted with a carbocyclyl or heterocyclyl group;
R² and R^{2′} are each independently selected from H and C₁ to C₆ alkyl;
R³ is selected from C₁ to C₆ alkyl, aryl, heteroaryl, substituted aryl, substituted heteroaryl, carbocyclyl, substituted carbocyclyl, heterocyclyl and substituted heterocyclyl, optionally wherein the C₁ to C₆ alkyl is substituted with one or more heteroatoms selected from halo, N, O and S and/or is substituted with a carbocyclyl or heterocyclyl group;
R⁴ is H, C₁-C₆ alkyl, optionally wherein the C₁-C₆ alkyl is substituted with one or more heteroatoms selected from N, O or S;
or wherein R¹ and R⁴ together form a 5-, 6-, or 7-membered heterocyclic ring;
or wherein R¹ and R² together form a 5-, 6-, or 7-membered heterocyclic ring;
or wherein R² and R⁴ together form a 5-, 6-, or 7-membered heterocyclic or carbocyclic ring; and
L shows the position of attachment of the linker.

As shown in formula (II) above, the linker is appended to moiety Z via the aromatic ring. In particular, the linker is attached to moiety Z by way of a covalent bond between an atom on the linker and a carbon atom of the aryl ring system. The linker may be attached to the aromatic ring at any position (provided it has the correct valency and/or is chemically suitable). For example, the linker may replace a hydrogen atom at any position on the aromatic ring.

A representative example of a compound according to formula (II) includes, but is not limited to:

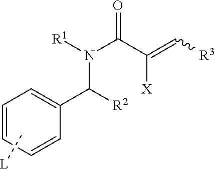

Wherein R³ and L are as defined for formulae (I) and (II) herein;
R¹ is selected from C₁ to C₆ alkyl; and
R² is selected from C₁ to C₆ alkyl.

In some cases, R¹ is methyl and R² is n-propyl.

In certain examples, when R¹ and R⁴ together form a 5-, 6-, or 7-membered heterocyclic ring, Z may be represented as formula (IIaa):

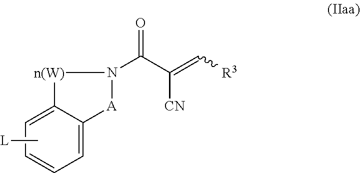

Wherein A, R³ and L are as defined for formulae (I) and (II) herein;
n is 1, 2 or 3; and
W is selected from CR^{W1}R^{W2}, O, NR^{W3} and S; and
R^{W1}, R^{W2} and R^{W3}are each independently selected from H and optionally substituted C₁ to C₆ alkyl; and
wherein when n is 2 or 3, each W is independently selected from CR^{W1}R^{W2}, O, NR^{W3}, and S.

In some cases, each W is CR^{W1}R^{W2}.

Representative examples of compounds according to formula (IIaa) include, but are not limited to:

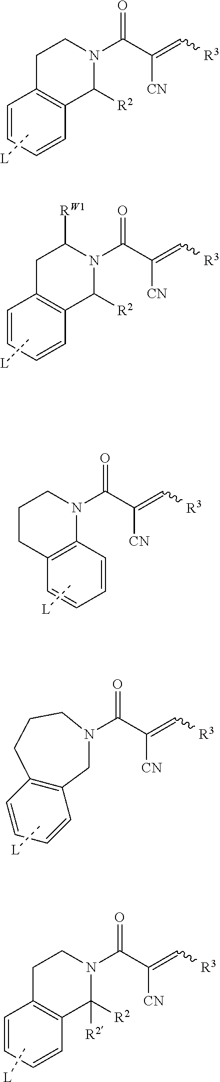

Wherein R³ and L are as defined herein for formula (I) above;
R² may be selected from H or C₁-C₆ alkyl optionally substituted with one or more heteroatoms selected from halo (such as methyl, ethyl, iso-propyl, or trifluoromethyl); R^{2′} may be C₁-C₆ alkyl (such as methyl); and
R^{W1} may be selected from C₁-C₆ alkyl (such as methyl or ethyl).

Representative examples of compounds according to formula (IIaa) include, but are not limited to:

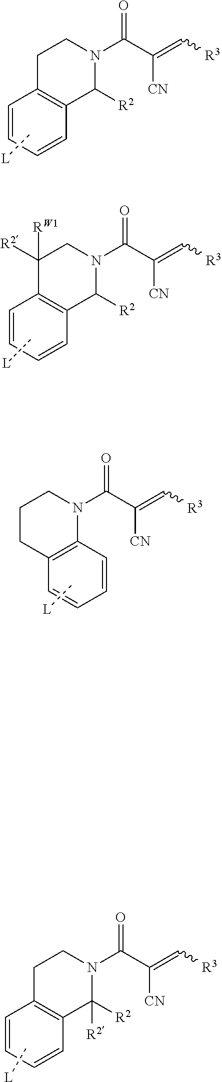

Wherein R³ and L are as defined herein for formula (I) above;
R² may be selected from H, or C₃-C₆ cycloalkyl, C₁-C₆ alkyl optionally substituted with C₁-C₄ alkoxy, or one or more heteroatoms selected from halo (such as cyclopropyl, methyl, ethyl, n-propyl, iso-propyl, methylmethoxy, difluoromethyl or trifluoromethyl); R^{2′} may be C₁-C₆ alkyl (such as methyl) or C₁-C₄ alkoxy (such as methoxy); and
R^{W1} may be selected from C₁-C₆ alkyl (such as methyl or ethyl).

By way of further example, when R¹ and R⁴ together form a 5-, 6-, or 7-membered heterocyclic ring, Z may be represented as formula (IIa):

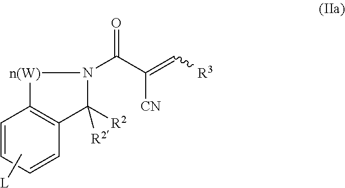

Wherein R², R^{2′}, R³ and L are as defined above, e.g. as for formula (II);
n is 1, 2 or 3; and
W is selected from CR^{W1}R^{W2}, O, NR^{W3} and S; and
R^{W1}, R^{W2} and R^{W3} are each independently selected from H and C₁ to C₆ alkyl; and
wherein when n is 2 or 3, each W is independently selected from CR^{W1}R^{W2}, O, NR^{W3}, and S.

In some cases, each W is CH₂.

In some examples, Z may be represented as formula (IIa′):

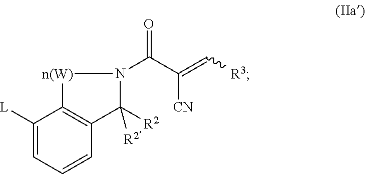

Wherein R³ and L are as defined above;
R² is C₁ to C₃ alkyl; R^{2′} is H; n is 2; and each W is CH₂.

By way of yet further example, Z may be selected from one of the following structures:

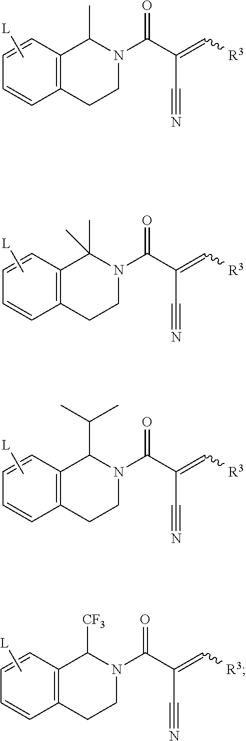

wherein R³ and L are as defined above and herein.

Alternatively, Z may be selected from one of the following structures:

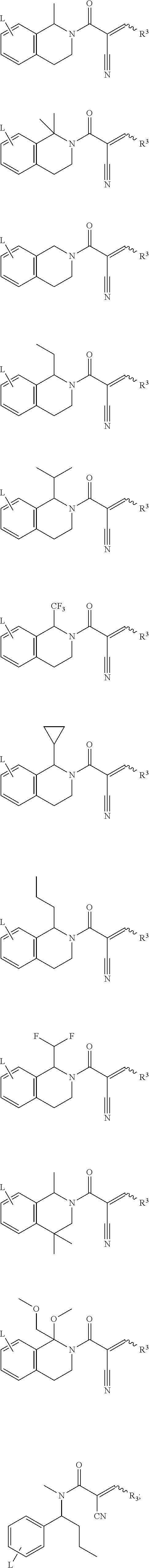

wherein R³ and L are as defined above and herein.

The present invention also relates to any compound comprising a moiety selected from one of the following structures:

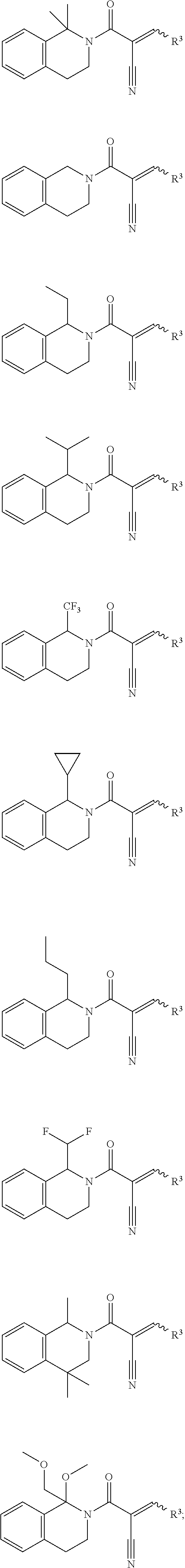

wherein R³ is as defined above and herein.

The present invention also relates to a compound selected from one of the following structures:

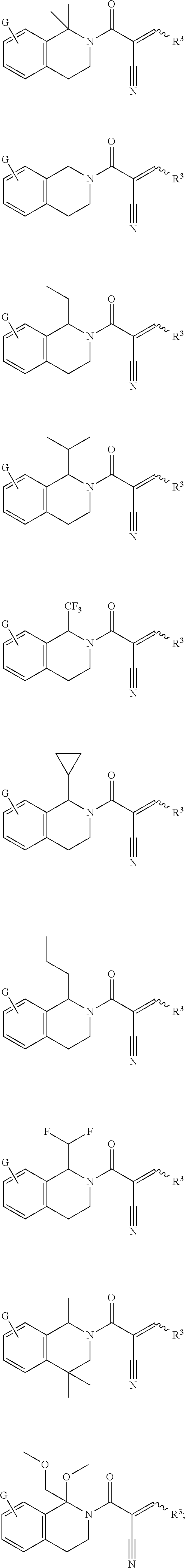

wherein R³ is as defined above and herein.

The group G is configured to enable attachment of the compound to another chemical structure (such as a linker moiety or a linker-target protein binding ligand moiety) via formation of a new covalent bond. Following the formation of this new covalent bond, the group G may form part of a linker as defined herein.

In some examples, G may comprise a functional group that is able to facilitate the formation of a new covalent bond between Z and another moiety, e.g. via formation of an amide, ester, thioester, keto, urethane, amine, or ether linkage, or via formation of a new carbon-carbon bond or new carbon-nitrogen bond.

By way of example only, G may be represented as shown below:

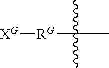

wherein R^{G} is absent or is a C₁ to C₆ alkyl, optionally substituted with one or more heteroatoms selected from N, O and S;
X^{G} is a group that is selected from —CO₂H, —(CO)—N-hydroxysuccinimide and —(CO)-pentafluorophenol esters, —CHO, —COR^{G1}, —OH, —NH₂, —NHR^{G2}, halo (e.g. iodo and bromo), O-leaving group (such as —OTs (tosylate), OMs (mesylate), —OTf (triflate)), alkynyl, azide, dienyl, aminoxy, tetrazinyl, (E)-cyclooctenyl, cyclooctynyl, norbornyl, boronic acid, boronate ester, alkylboranes or an organometallic group (e.g. organotin, zinc or other suitable reagent); and
R^{G1} and R^{G2} are each independently selected from C₁ to C₆ alkyl.

In this structure, a wavy line is shown over the bond that forms the link with the aromatic moiety of the compound.

G is linked to the aromatic moiety of the compound by way of the R^{G} group. In those cases where R^{G} is absent, the group X^{G} is directly attached to the aromatic moiety of the compound.

Representative examples of suitable G moieties are shown below:

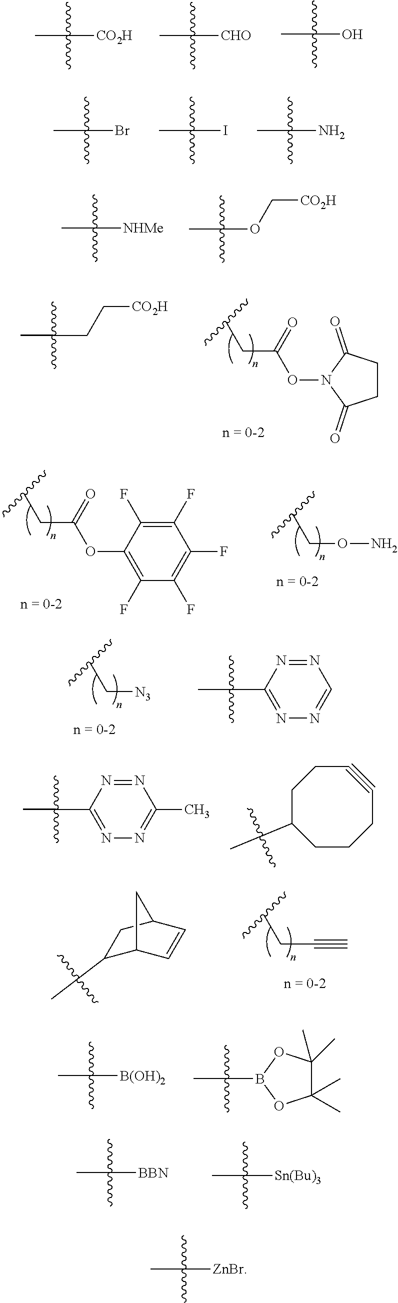

When R¹ and R² together form a 5-, 6-, or 7-membered heterocyclic ring, Z may be represented as formula (IIb):

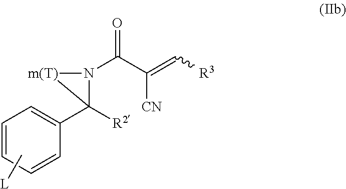

Wherein R^{2′}, R³ and L are as defined above, e.g. as for formula (II);
m is 3, 4 or 5;
each T is independently selected from CR^{T1}R^{T2}, O, NR^{T3} and S; and
R^{T1}, R^{T2} and R^{T3} are each independently selected from H and C₁ to C₆ alkyl.

For example, in some cases, each T is CH₂.

When R² and R⁴ together form a 5-, 6-, or 7-membered heterocyclic or carbocyclic ring, Z may be represented as formula (IIc):

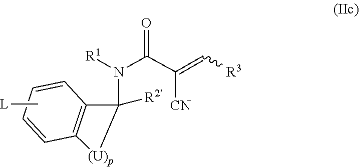

Wherein R¹, R^{2′}, R³ and L are as defined above, e.g. as for formula (II);
p is 2, 3 or 4; and
each U is independently selected from CR^{U1}R^{U2}, O, NR^{U3} and S; and
R^{U1}, R^{U2} and R^{U3} are each independently selected from H and C₁ to C₆ alkyl.

For example, in some cases, each T is CH₂.

Representative examples of Z are shown below:

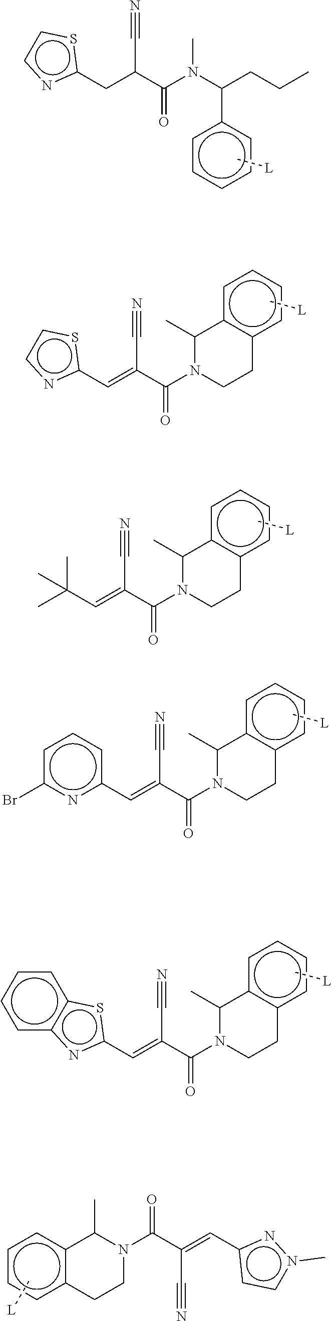

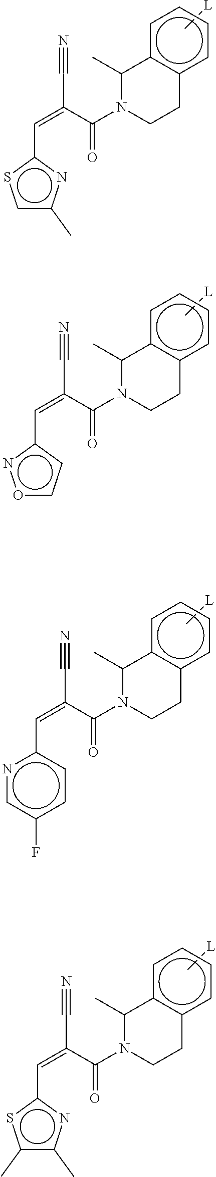

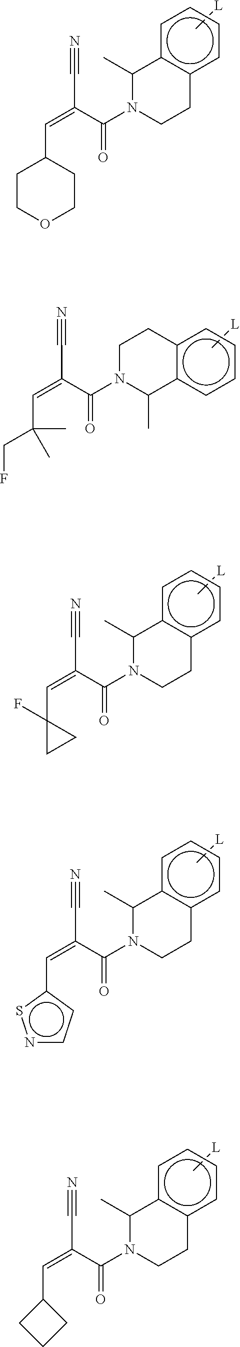

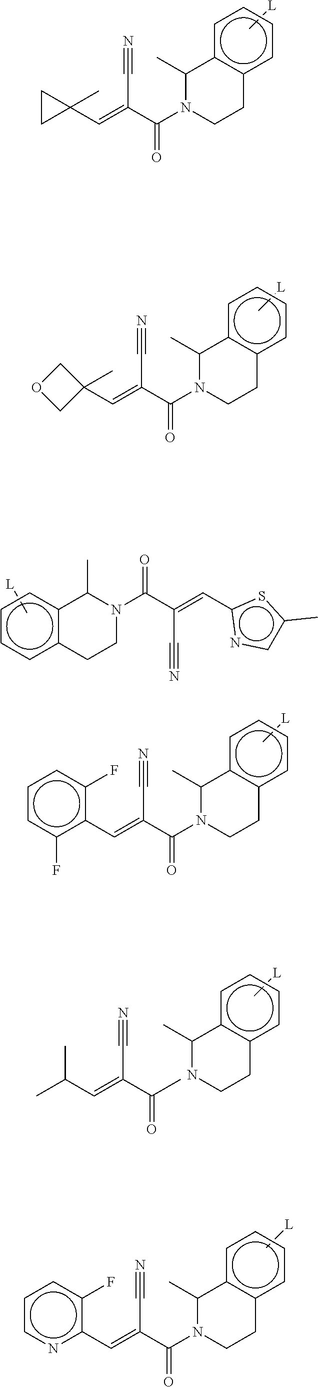

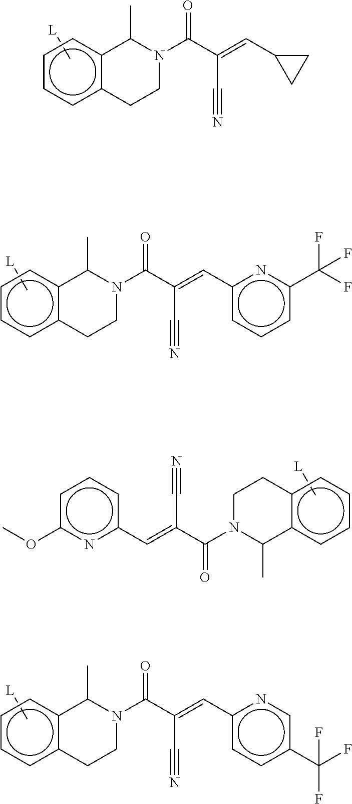

Further representative examples of Z are shown below:

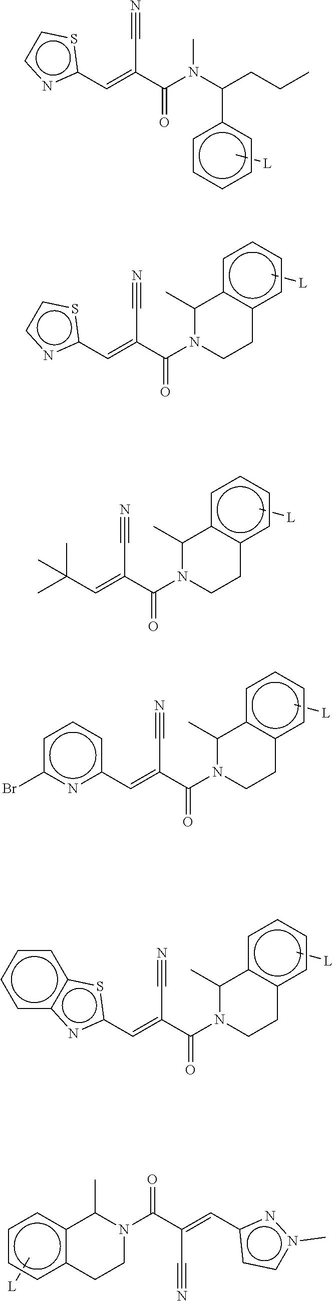

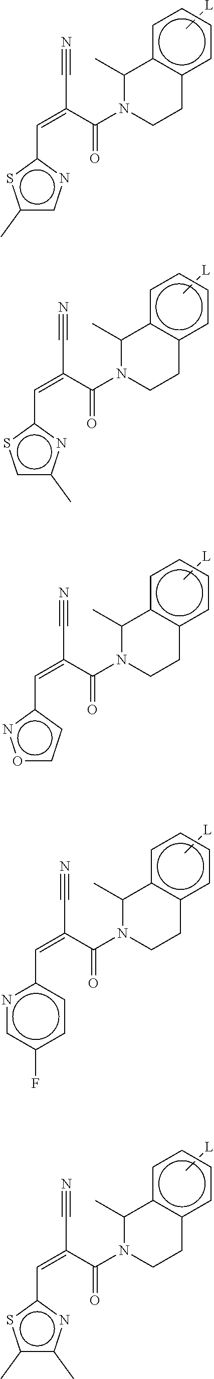

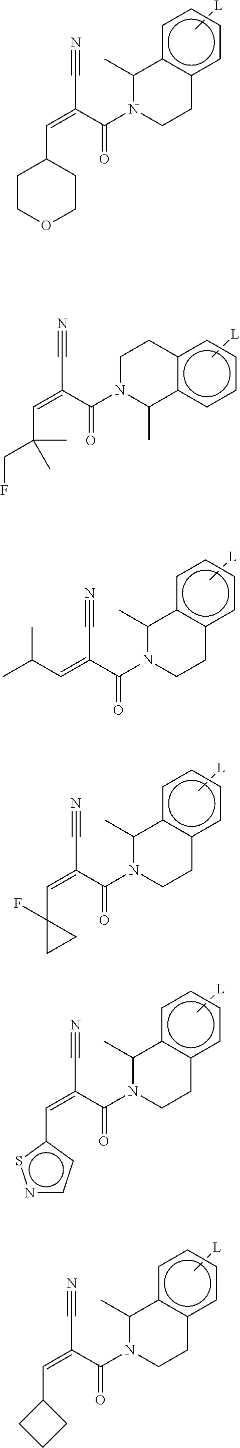

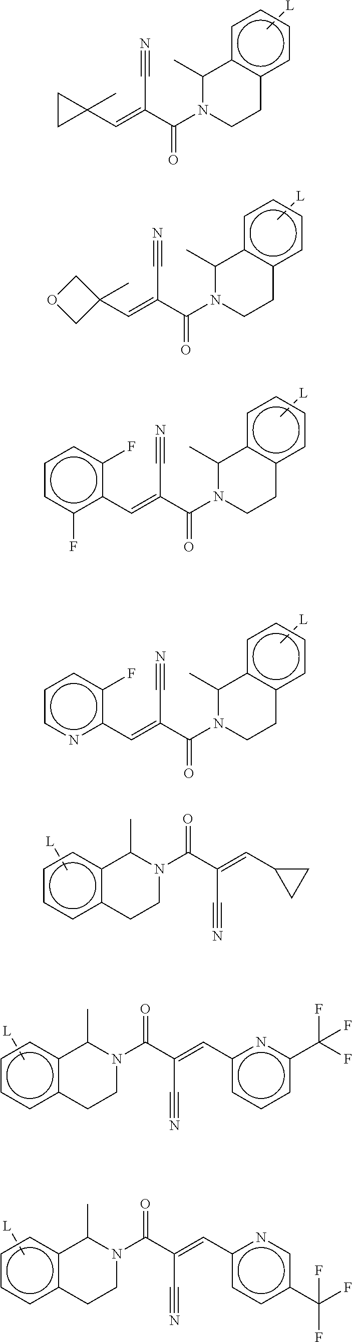

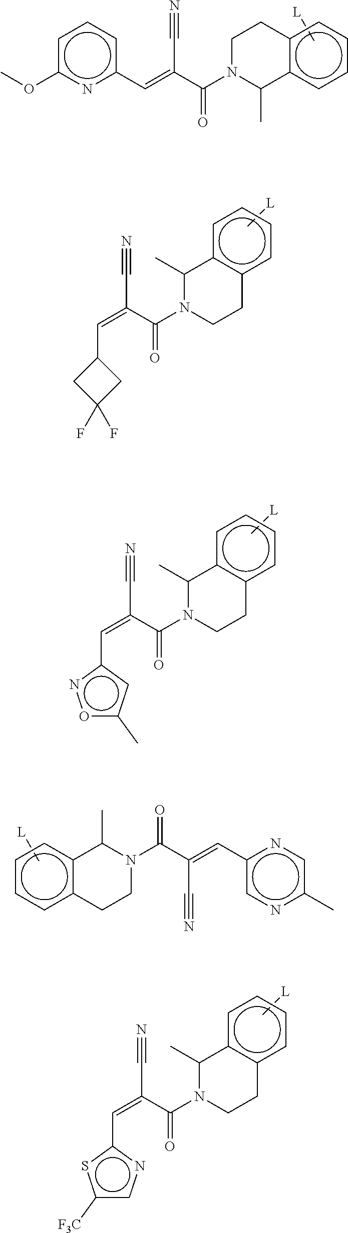

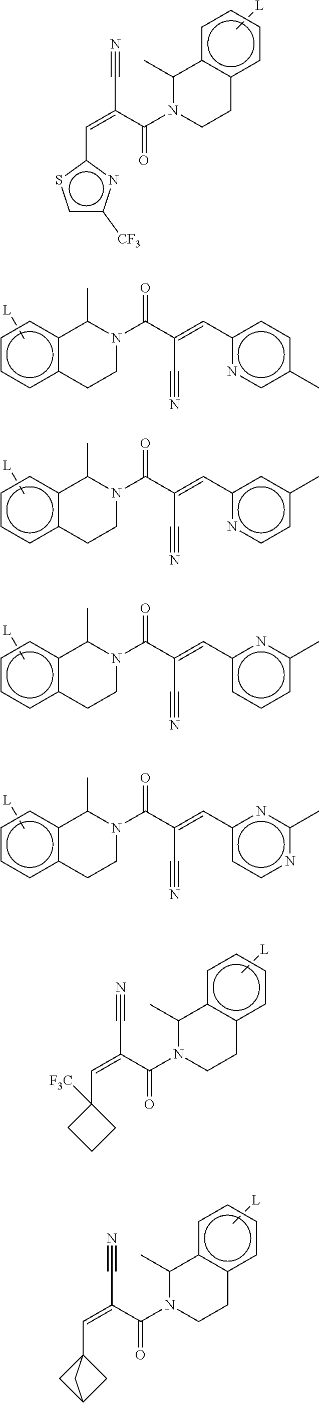

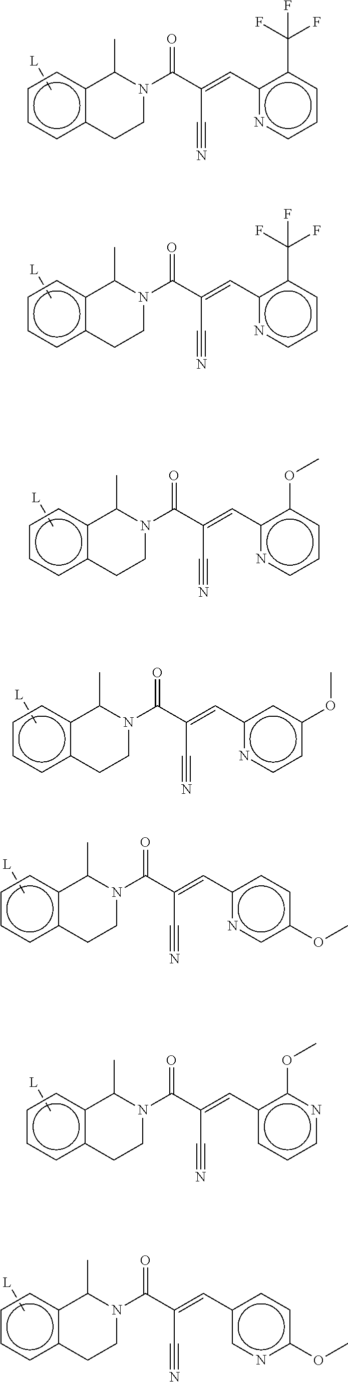

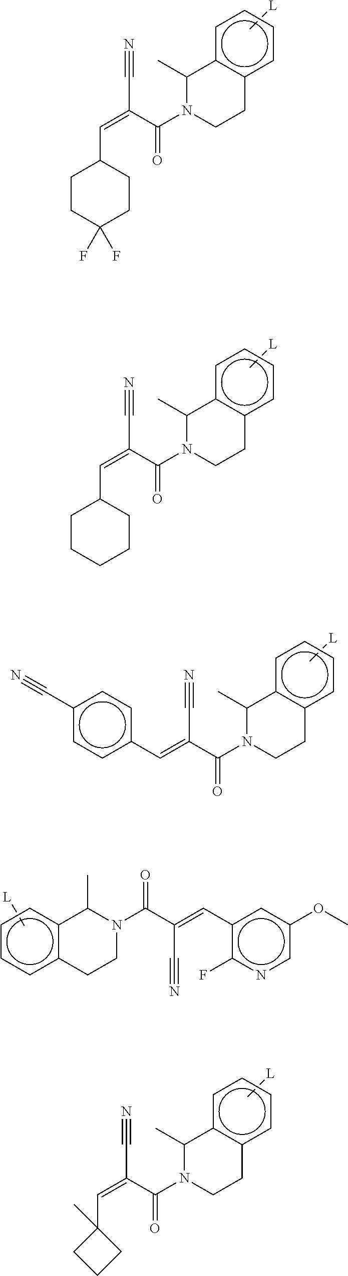

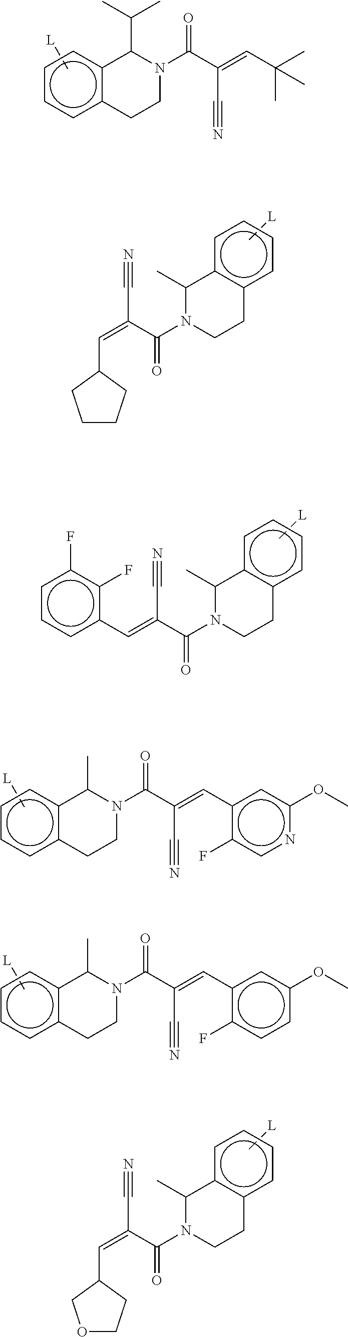

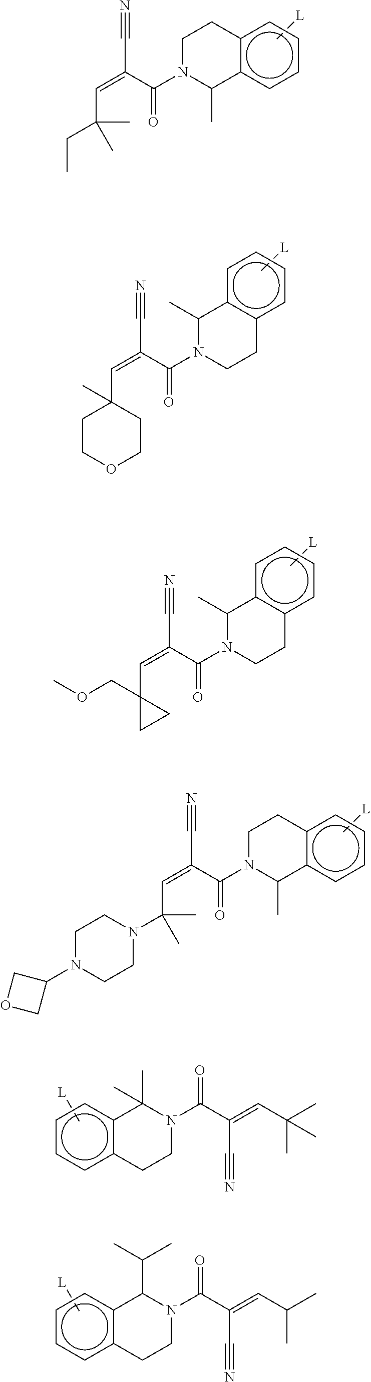

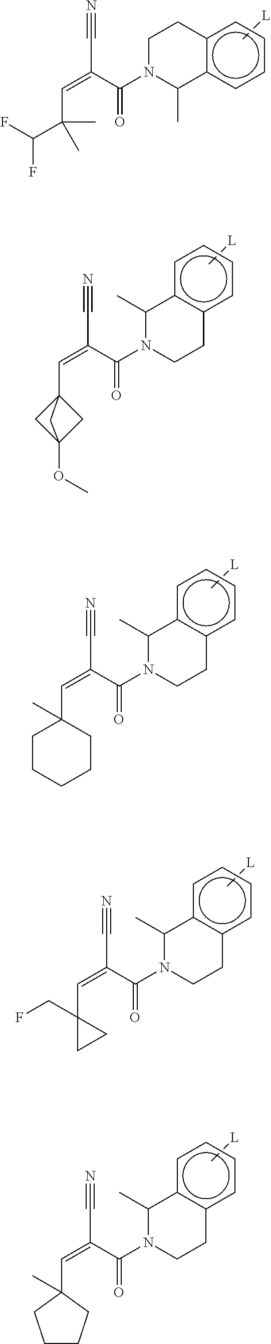

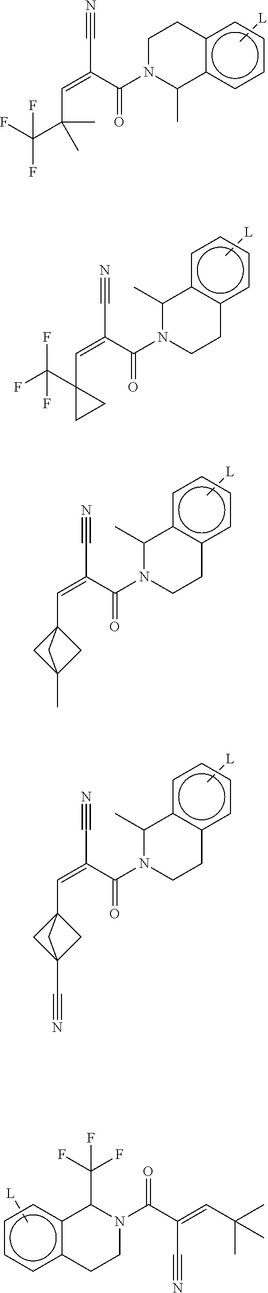

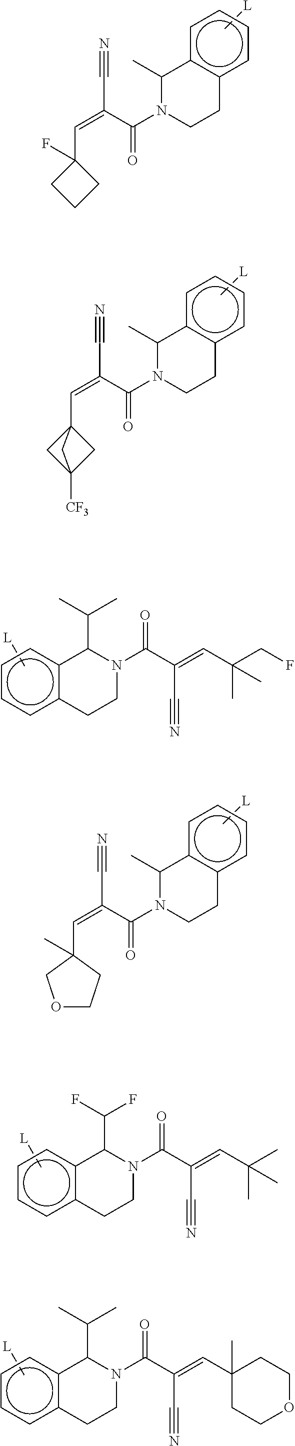

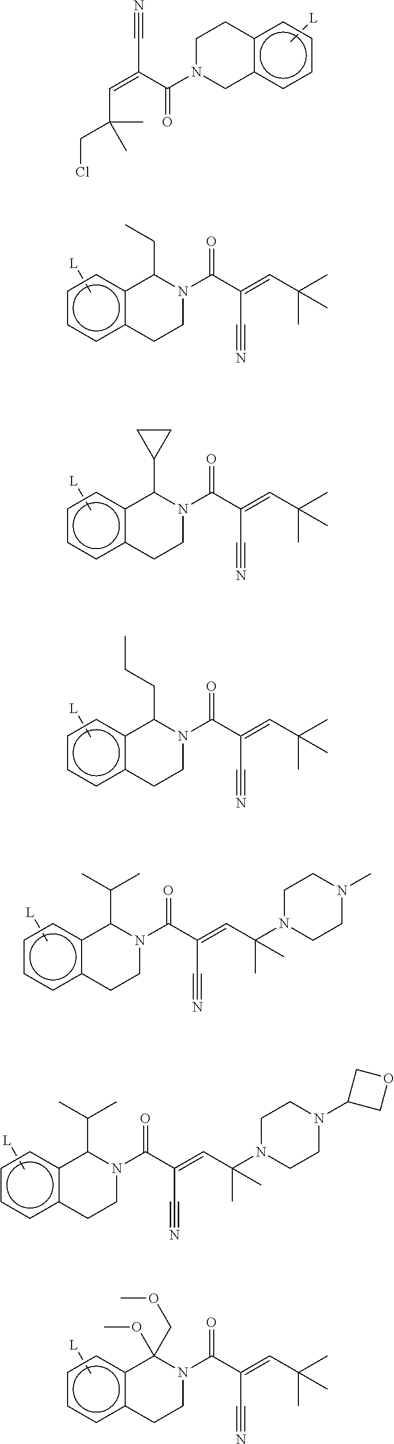

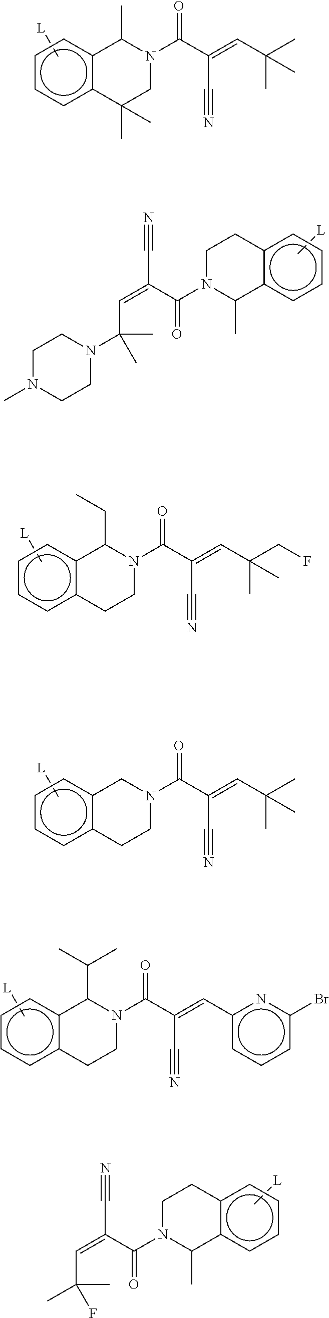

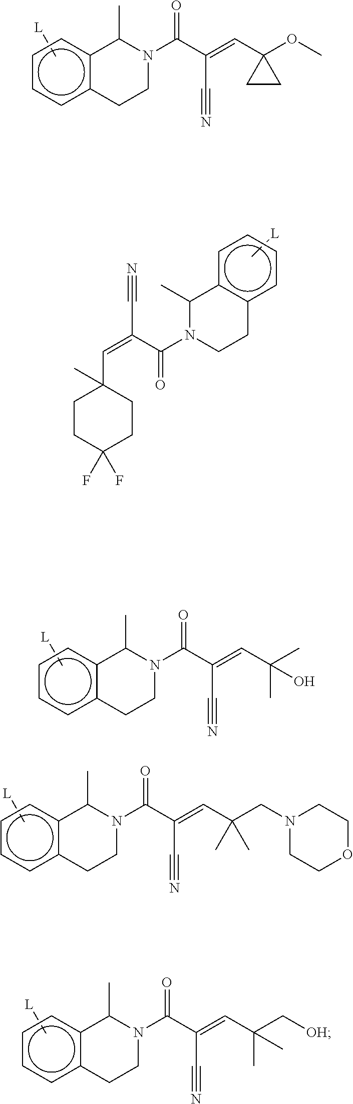

The dotted line on the structures above indicates that the linker may be joined to the Z moiety at any position on the aromatic ring (provided that it has the correct valency and/or is chemically suitable). For example, the linker may replace a hydrogen atom at any position on the aromatic ring. By way of further example, in cases where B is a phenyl ring, the linker may be attached in a para-substitution pattern with the pendant amide group as illustrated in formula (IId) below.

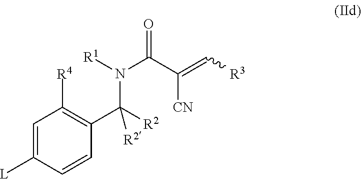

Alternatively it is noted, that whilst the formulae (I) to (IId) indicate that the linker is joined to the Z moiety via ring B (which may in some cases be an aromatic ring), the present disclosure also extends to examples wherein the linker is attached at any other position in the Z moiety (provided that it has the correct valency and/or is chemically suitable). For example, the linker may replace a hydrogen atom at any position in the Z moiety. Thus, in some examples, Z may be represented as shown in formulae (III):

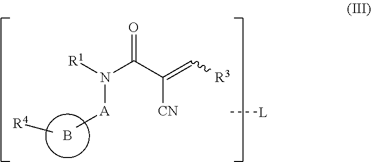

wherein R¹, A, R³, R⁴, B and L are as defined for formula (I) (or any of formulae (Ia) to (IId)).

The dotted line shown through the square brackets on formula (III) indicates that the linker may be joined via a covalent bond to any atom on the Z moiety provided that it has the correct valency, is chemically suitable and/or provided that the attachment of the linker at this alternative position does not disrupt the function of the Z moiety in promoting and/or facilitating proteasomal degradation.

The Z moiety may, in some embodiments, not be:

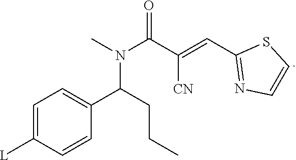

In some embodiments, the Z moiety may, for example, be of formula (Ia), (Ib), (IIaa), (IIa) or (IIb).

The inventors have found that certain exemplary bifunctional molecules comprising Z moieties of formulae (Ia), (Ib), (IIaa), (IIa) or (IIb) can be used to more selectively degrade BRD9 over other proteins, such as other BRD proteins, e.g. BRD4.

As described above, Z may comprise a structure according to formula (I), formula (WZI), or formula (WI).

Formula (WZI) is:

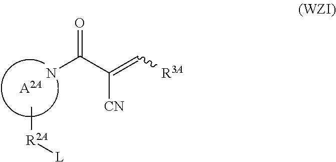

wherein:
ring A^{2A} is an optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from N, O and S;
R^{2A} is absent or is selected from aryl, substituted aryl, heteroaryl, substituted heteroaryl, heterocycloalkyl, substituted heterocycloalkyl, NR^{y}, —CH(aryl)-, —CH(substituted aryl)-, —CH(heteroaryl)- and —CH(substituted heteroaryl)-;
wherein R^{y} is optionally substituted C₁₋₆alkyl or H;
R^{3A} is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S; and
L shows the point of attachment of the linker;
Formula (WI) is:

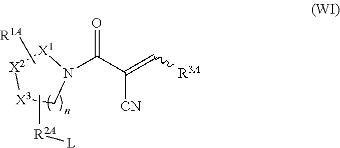

wherein R^{1A} is absent or is selected from aryl, substituted aryl, heteroaryl, substituted heteroaryl, cycloalkyl, C₁ to C₆ alkyl and substituted C₁ to C₆ alkyl;
R^{2A} is absent or is selected from aryl, substituted aryl, heteroaryl, substituted heteroaryl, heterocycloalkyl, substituted heterocycloalkyl, —CH(aryl)-, —CH(substituted aryl)-, —CH(heteroaryl)- and —CH(substituted heteroaryl)-;
R^{3A} is selected from C₁ to C₆ alkyl, substituted C₁ to C₆ alkyl, aryl, substituted aryl, heteroaryl, and substituted heteroaryl;
X¹ is CH₂;
X² and X³ are each independently CH₂, or a heteroatom selected from O and NR^{x}, wherein R^{x} is H or C₁ to C₆ alkyl; and
n is 0, 1, 2, or 3; and
L shows the point of attachment of the linker.

In some examples, when Z is of formula (WZI) or formula (WI) or any sub-generic formulae described below, it may not be:

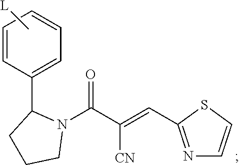

In embodiments of formula (WI), at least one of R^{1A} and R^{2A} is present.

As shown in formula (WZI) above, the linker may be appended to moiety Z via the R^{2A} group. In such examples, the linker may be attached to moiety Z by way of a covalent bond between an atom on the linker and an atom contained in the ring system of the aryl, substituted aryl, heteroaryl, substituted heteroaryl, heterocycloalkyl or substituted heterocycloalkyl of the R^{2A} group. Alternatively, the linker may be attached to moiety Z by way of a covalent bond to the nitrogen atom of NR^{y} or the benzylic carbon atom of the —CH(aryl)- or —CH(substituted aryl)-, for example by way of a covalent bond to the benzylic carbon atom of the —CH(aryl)- or —CH(substituted aryl)-.

As described above, in some examples of formula (WZI) or formula (WI), R^{2A} may be absent. In such examples, the linker may be appended to moiety Z by way of a covalent bond between an atom on the linker and an atom contained in the heterocyclic ring (e.g. ring A^{2A}).

In all of the examples, the linker may be attached at any suitable position e.g. provided it has the correct valency and/or is chemically suitable. For example, the linker may be bonded at any position on the aryl, substituted aryl, heteroaryl, substituted heteroaryl, heterocycloalkyl, substituted heterocycloalkyl, NR^{y}, —CH(aryl)- or —CH(substituted aryl)- of the R^{2A} group or at any position on the heterocyclic ring shown, for example, in formula (WZI) or formula (WI).

As described above, ring A^{2A} is an optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from N, O and S, such as N and O.

When ring A^{2A} is bicyclic or tricyclic, and unless otherwise stated, it may comprise rings that are joined by a bond, rings that are fused, a bridged ring and/or rings that are joined at a spiro centre.

When ring A^{2A} is bicyclic, it may be a bridged bicyclic ring (i.e. it may comprise two rings that share three or more atoms) or it may be a spirocyclic bicyclic ring (i.e. it may comprise two rings that share one atom, e.g. the two rings may be joined at a spiro centre).

When ring A^{2A} is a bridged bicyclic ring, it may be an optionally substituted 7- to 12-membered bridged bicyclic N-heterocycloalkyl optionally containing one or two additional ring heteroatoms selected from N, O and S. In some examples, ring A^{2A} is a 7- or 8-membered bridged bicyclic N-heterocycloalkyl optionally containing one or two additional ring heteroatoms selected from N, O and S. In some examples, ring A^{2A} is a 7- or 8-membered bridged bicyclic N-heterocycloalkyl optionally containing one additional ring atom selected from N.

When ring A^{2A} is a spirocyclic bicyclic ring, it may be an optionally substituted 7- to 12-membered spirocyclic bicyclic N-heterocycloalkyl optionally containing one or two additional ring heteroatoms selected from N, O and S. In some examples, ring A^{2A} is a 7- to 12-membered spirocyclic bicyclic N-heterocycloalkyl optionally containing one or two additional ring heteroatoms selected from N, O and S. In some cases, ring A^{2A} is bicyclic and comprises a first 5- to 7-membered ring and a second 3- to 7-membered ring. For example, ring A^{2A} may be a spirocyclic bicyclic N-heterocycloalkyl comprising a first 5- or 6-membered ring and a second 3- to 6-membered ring, and optionally containing one or two additional ring heteroatoms selected from N, O and S. In some examples, ring A^{2A} may be a spirocyclic bicyclic N-heterocycloalkyl comprising a first 5- or 6-membered ring and a second 3- to 6-membered ring, and optionally containing one additional ring heteroatoms selected from N.

In some embodiments, Z comprises a structure according to formula (WZIa):

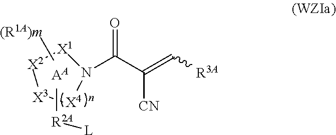

wherein:
R^{1A} is absent (i.e. when m is 0) or is selected from aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R^{1A} groups combine to form an optionally substituted C₁₋₃ bridge, optionally substituted C₃₋₅cycloalkyl or optionally substituted 5- to 7-membered heterocycloalkyl (e.g. 5- to 7-membered N-heterocycloalkyl), optionally wherein the C₃₋₅cycloalkyl or the 5- to 7-membered heterocycloalkyl are joined to ring A^{A} at a spiro centre;
R^{2A} is absent or is selected from aryl, substituted aryl, heteroaryl, substituted heteroaryl, heterocycloalkyl, substituted heterocycloalkyl, NR^{y}, —CH(aryl)-, —CH(substituted aryl)-, —CH(heteroaryl)- and —CH(substituted heteroaryl)-;
wherein R^{y} is optionally substituted C₁₋₆alkyl or H;
R^{3A} is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S;
X¹ is CH₂;
X², X³ and X⁴ are each independently CH₂, O or NR^{x};
R^{x} is H or C₁ to C₆ alkyl, or wherein one R^{1A} group and one R^{x} group combine to form an optionally substituted C₁₋₃ bridge;
n is 0, 1, 2, or 3;
m is 0, 1, 2, 3 or 4; and
L shows the point of attachment of the linker.

In some examples, where n is 1, 2 or 3 (i.e. when 1, 2 or 3 X⁴ groups are present), an X⁴ group adjacent to (or directly bonded to) the N of the heterocyclic ring shown in formula (WZIa) is CH₂.

In some examples, Z comprises a structure according to formula (WZIb):

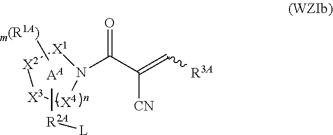

wherein:
R^{1A} is absent (i.e. when m is 0) or is selected from aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R^{1A} groups combine to form an optionally substituted C₁₋₃ bridge, optionally substituted C₃₋₅cycloalkyl or optionally substituted 5- to 7-membered heterocycloalkyl (e.g. a 5- to 7-membered N-heterocycloalkyl), optionally wherein the C₃₋₅cycloalkyl or the 5- to 7-membered heterocycloalkyl are joined to ring A^{A} at a spiro centre;
R^{2A} is absent or is selected from aryl, substituted aryl, heteroaryl, substituted heteroaryl, heterocycloalkyl, substituted heterocycloalkyl, NR^{y}, —CH(aryl)-, —CH(substituted aryl)-, —CH(heteroaryl)- and —CH(substituted heteroaryl)-;
wherein R^{y} is optionally substituted C₁₋₆alkyl or H;
R^{3A} is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S;
X¹ and X⁴ are each CH₂;
X² and X³ are each independently CH₂. O or NR^{x}; with the proviso that none or only 1 of X² and
X³ is O;
R^{x} is H or C₁ to C₆ alkyl; or wherein one R^{1A} group and one R^{x} group combine to form an optionally substituted C₁₋₃ bridge; n is 0, 1, 2 or 3;
m is 0, 1, 2, 3 or 4; and
L shows the point of attachment of the linker.

In some examples, Z comprises a structure according to formula (WZIb′):

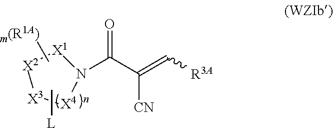

wherein:
R^{1A}, R^{3A}, X¹, X², X³, X⁴, n, m and L are as defined above in respect of formula (WZIa) and (WZIb).

In some examples, Z comprises a structure according to formula (WZIb″):

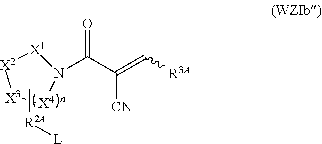

wherein:
R^{2A}, R^{3A}, X¹, X², X³, X⁴, n and L are as defined above in respect of formula (WZIa) and (WZIb).

As stated above, in some embodiments of formulae (WZIa), (WZIb), (WZIb′), and (WZIb″) (and other formulae as described herein), an optionally substituted C₁₋₃ bridge may be formed by two R^{1A} groups or, in some cases, by one R^{1A}group and one R^{x} group. The C₁₋₃ bridge may be a C₁-C₃ alkylene bridging group, such as methylene, ethylene or propylene. In some examples, the C₁-C₃ bridge may be methylene or ethylene. Where the C₁₋₃ bridge is substituted, it may comprise from one to three (e.g. one or two) substituents (selected from any suitable substituent as described herein). For example, the C₁ to C₃ alkylene bridging group may be optionally substituted with one or two substituents each independently selected from the group consisting of halo, C₁ to C₃ alkyl, C₁ to C₃ haloalkyl and C₁ to C₃alkoxy.

In further embodiments, Z may comprise a structure according to formula (WI):

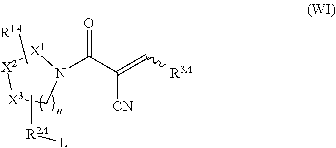

wherein R^{1A} is absent or is selected from aryl, substituted aryl, heteroaryl, substituted heteroaryl, cycloalkyl, C₁ to C₆ alkyl and substituted C₁ to C₆ alkyl;
R^{2A} is absent or is selected from aryl, substituted aryl, heteroaryl, substituted heteroaryl, heterocycloalkyl, substituted heterocycloalkyl, —NR^{y}, —CH(aryl)-, —CH(substituted aryl)-, —CH(heteroaryl)- and —CH(substituted heteroaryl)-;
wherein R^{y} is H or C₁ to C₆ alkyl;
R^{3A} is selected from C₁ to C₆ alkyl, substituted C₁ to C₆ alkyl, aryl, substituted aryl, heteroaryl, and substituted heteroaryl;
X¹ is CH₂;
X² and X³ are each independently CH₂, or a heteroatom selected from O and NR^{x}, wherein R^{x} is H or C₁ to C₆ alkyl;
n is 0, 1, 2, or 3; and
L shows the point of attachment of the linker;
and further wherein Z is not:

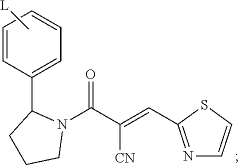

In alternative examples of formula (WI), the list of options for R^{3A} given above, may be replaced with is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S.

In some embodiments, R^{2A} may be absent or selected from aryl, substituted aryl, heteroaryl, substituted heteroaryl, heterocycloalkyl, substituted heterocycloalkyl, —CH(aryl)-, —CH(substituted aryl)-, —CH(heteroaryl)- and —CH(substituted heteroaryl)-.

In some examples of formula (WI), at least one of R^{1A} or R^{2A} is present.

For example, where R^{1A} is absent, R^{2A} may be present and selected from aryl, substituted aryl, heteroaryl, substituted heteroaryl, heterocycloalkyl, substituted heterocycloalkyl, —NR^{y}, —CH(aryl)-, —CH(substituted aryl)-, —CH(heteroaryl)- and —CH(substituted heteroaryl)-. For example, where R¹ is absent, R² may be present and selected from aryl, substituted aryl, heteroaryl, substituted heteroaryl, heterocycloalkyl, substituted heterocycloalkyl, —CH(aryl)-, —CH(substituted aryl)-, —CH(heteroaryl)- and —CH(substituted heteroaryl)-.

By way of further example, where R^{2A} is absent, R^{1A} may be present and selected from aryl, substituted aryl, heteroaryl, substituted heteroaryl, cycloalkyl, substituted cycloalkyl, C₁ to C₆ alkyl and substituted C₁ to C₆ alkyl. By way of even further example, where R^{2A} is absent, at least one R^{1A} may be selected from aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R^{1A} groups combine to form an optionally substituted C₁₋₃ bridge, optionally substituted C₃₋₆cycloalkyl or optionally substituted 5- to 7-membered N-heterocycloalkyl, optionally wherein the C₃₋₅cycloalkyl or the 5-7-membered N-heterocycloalkyl are joined to ring A^{A} at a spiro centre.

In some examples of formula (WI), both of R^{1A} and R^{2A} are present. For example, in some cases, R^{2A} is present and at least one R^{1A} is selected from aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R^{1A} groups combine to form a optionally substituted C₁₋₃ bridge, optionally substituted C₃₋₆cycloalkyl or optionally substituted 5- to 7-membered N-heterocycloalkyl.

In compounds of formula (WZI) and formula (WI) (and sub-formulae thereof), R^{1A} and/or R^{2A} may be covalently attached to the heterocyclic ring (e.g. ring A^{2A} or ring A^{A}) at any suitable position e.g. provided it has the correct valency and/or is chemically suitable. For example, R^{1A} and/or R^{2A} may replace a hydrogen atom at any position on the heterocyclic core, e.g. that shown in formula (WI).

Where both R^{1A} and R^{2A} are present, they may be covalently attached to the heterocyclic ring (e.g. ring A^{2A} or ring A^{A}) at the same or different positions. For example, in some cases R^{1A} and R^{2A} may be covalently attached to the heterocyclic core by way of different carbon atoms. In other cases, R^{1A} and R^{2A} may be covalently attached to the heterocyclic core by way of the same carbon atom.

By way of further example, Z may be represented as either formula (WIa) or (WIb):

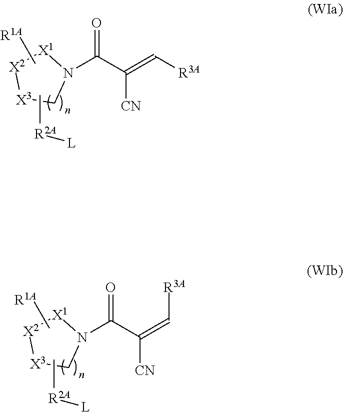

wherein R^{1A}, R^{2A}, R^{3A}, X¹, X², X³ and n are as defined above and herein with respect to formula (WI) and its subgeneric formulae set out below.

By way of further example, Z may be represented as formula (WIc′):

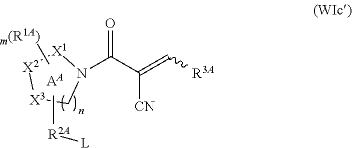

wherein:
   R^{1A} is absent (i.e. m is 0) or is selected from the group consisting of: aryl having 6 to 10 carbon ring atoms that is optionally substituted with one to three substituents; heteroaryl having 5 to 10 ring atoms containing 1 to 3 heteroatoms each independently selected from N, O and S, the heteroaryl being optionally substituted with one to three substituents; C₃ to C₈ cycloalkyl being optionally substituted with one to three substituents; heterocycloalkyl having 3 to 10 ring atoms and containing 1 to 3 ring heteroatoms each independently selected from N, O and S, the heterocycloalkyl being optionally substituted with one to three substituents; C₁ to C₆ alkyl optionally substituted with one to three substituents; and/or wherein two R^{1A} groups combine to form a C₁₋₃ bridge optionally substituted with one to three substituents, C₃₋₅cycloalkyl optionally substituted with one to three substituents or 5- to 7-membered N-heterocycloalkyl optionally substituted with one to three substituents (e.g wherein the C₃₋₅cycloalkyl or the 5-7-membered N-heterocycloalkyl are joined to ring A^{A} at a spiro centre);
R^{2A} is absent or is selected from the group consisting of: aryl having 6 to 10 carbon ring atoms, the aryl being optionally substituted with one to three substituents; heteroaryl having 5 to 10 ring atoms and containing 1 to 3 heteroatoms each independently selected from N, O and S, the heteroaryl being optionally substituted with one to three substituents; heterocycloalkyl having 3 to 10 ring atoms and containing 1 to 3 heteroatoms each independently selected from N, O and S, the heterocycloalkyl being optionally substituted with one to three substituents; —NR^{y}; —CH(aryl)-, wherein the aryl has 6 to 10 carbon ring atoms and is optionally substituted with one to three substituents)-; and —CH(heteroaryl)-, wherein the heteroaryl has 5 to 10 ring atoms and contains 1 to 3 heteroatoms each independently selected from N, O and S, the heteroaryl being optionally substituted with one to three substituents;
   wherein R^{y} is H or C₁ to C₆ alkyl;
   R^{3A} is selected from the group consisting of: C₁ to C₆ alkyl optionally substituted with one to three substituents; C₃ to C₈ cycloalkyl optionally substituted with one to three substituents; heterocycloalkyl having 3 to 10 ring atoms and containing 1 to 3 heteroatoms each independently selected from N, O and S, the heterocycloalkyl being optionally substituted with one to three substituents; aryl having 6 to 10 carbon ring atoms, the aryl being optionally substituted with one to three substituents; heteroaryl having 5 to 10 ring atoms and containing 1 to 3 heteroatoms each independently selected from N, O and S, the heteroaryl being optionally substituted with one to three substituents;
   X¹ is CH₂;
   X² and X³ are each independently CH₂, or a heteroatom selected from O and NR^{x}, wherein R^{x} is H or C₁ to C₆ alkyl, or wherein one R^{1A} group and one R^{x} group combine to form a C₁₋₃ bridge optionally substituted with one to three substituents; with the proviso that none, or only 1 or 2 X² and X³ is a heteroatom; and
   m is 0, 1, 2 or 3;
   n is 0, 1, 2, or 3; and
L shows the point of attachment of the linker.

By way of further example, Z may be represented as formula (WIc):

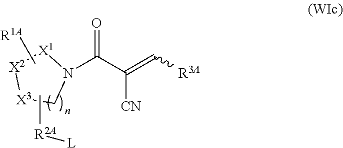

wherein:
R^{1A} is absent or is selected from the group consisting of: aryl having 6 to 10 carbon ring atoms that is optionally substituted with one to three substituents; heteroaryl having 5 to 10 ring atoms containing 1 to 3 heteroatoms each independently selected from N, O and S, the heteroaryl being optionally substituted with one to three substituents; C₃ to C₈ cycloalkyl; C₁ to C₆ alkyl optionally substituted with one to three substituents;
R^{2A} is absent or is selected from the group consisting of: aryl having 6 to 10 carbon ring atoms, the aryl being optionally substituted with one to three substituents; heteroaryl having 5 to 10 ring atoms and containing 1 to 3 heteroatoms each independently selected from N, O and S, the heteroaryl being optionally substituted with one to three substituents; heterocycloalkyl having 3 to 10 ring atoms and containing 1 to 3 heteroatoms each independently selected from N, O and S, the heterocycloalkyl being optionally substituted with one to three substituents; —NR^{y}; —CH(aryl)-, wherein the aryl has 6 to 10 carbon ring atoms and is optionally substituted with one to three substituents)-; and —CH(heteroaryl)-, wherein the heteroaryl has 5 to 10 ring atoms and contains 1 to 3 heteroatoms each independently selected from N, O and S, the heteroaryl being optionally substituted with one to three substituents;
wherein R^{y} is H or C₁ to C₆ alkyl;
R^{3A} is selected from the group consisting of: C₁ to C₆ alkyl optionally substituted with one to three substituents; C₃ to C₈ cycloalkyl optionally substituted with one to three substituents; heterocycloalkyl having 3 to 10 ring atoms and containing 1 to 3 heteroatoms each independently selected from N, O and S, the heterocycloalkyl being optionally substituted with one to three substituents; aryl having 6 to 10 carbon ring atoms, the aryl being optionally substituted with one to three substituents; heteroaryl having 5 to 10 ring atoms and containing 1 to 3 heteroatoms each independently selected from N, O and S, the heteroaryl being optionally substituted with one to three substituents;
X¹ is CH₂;
X² and X³ are each independently CH₂, or a heteroatom selected from O and NR^{x}, wherein R^{x} is H or C₁ to C₆ alkyl; with the proviso that none, or only 1 or 2 X² and X³ is a heteroatom; and
n is 0, 1, 2, or 3; and
L shows the point of attachment of the linker.

By way of further example, Z may be represented as formula (WId′):

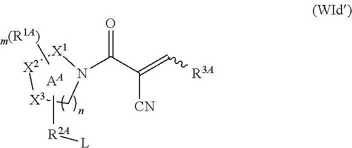

wherein:
   R^{1A} is absent (i.e. when m is 0) or is selected from the group consisting of: phenyl that is optionally substituted with one to three substituents selected from the group consisting of halo, C₁ to C₆ alkyl, C₁ to C₆ haloalkyl and C₁ to C₆ alkoxy; heteroaryl having 5 to 6 ring atoms containing 1 to 3 heteroatoms each independently selected from N, O and S, the heteroaryl being optionally substituted with one to three substituents selected from the group consisting of halo, C₁ to C₆ alkyl, C₁ to C₆ haloalkyl and C₁ to C₆ alkoxy; heterocycloalkyl having 5 to 7 ring atoms and containing 1 to 3 ring heteroatoms each independently selected from N, O and S; C₃ to C₈ cycloalkyl; C₁ to C₆ alkyl and C₁ to C₆ haloalkyl;
   and/or wherein two R^{1A} groups combine to form a C₁₋₃ bridge, C₃₋₅cycloalkyl or 5- to 7-membered N-heterocycloalkyl (e.g. wherein the C₃₋₅cycloalkyl or the 5-7-membered N-heterocycloalkyl are joined to ring A^{A} at a spiro centre);
   R^{2A} is absent or is selected from the group consisting o phenyl that is optionally substituted with one to three substituents selected from the group consisting of halo, C₁ to C₆ alkyl, C₁ to C₆ haloalkyl and C₁ to C₆ alkoxy; heteroaryl having 5 to 6 ring atoms containing 1 to 3 heteroatoms each independently selected from N, O and S, the heteroaryl being optionally substituted with one to three substituents each independently selected from the group consisting of halo, C₁ to C₆ alkyl, C₁ to C₆ haloalkyl and C₁ to C₆ alkoxy; heterocycloalkyl having 5 to 7 ring atoms and containing 1 to 3 heteroatoms each independently selected from N, O and S, the heterocycloalkyl being optionally substituted with one to three substituents each independently selected from the group consisting of halo, C₁ to C₆ alkyl, C₁ to C₆ haloalkyl and C₁ to C₆ alkoxy; —NR^{y}; —CH(phenyl)-, wherein the phenyl is optionally substituted with one to three substituents each independently selected from the group consisting of halo, C₁ to C₆ alkyl, C₁ to C₆ haloalkyl and C₁ to C₆ alkoxy; and —CH(heteroaryl), wherein the heteroaryl has 5 to 6 ring atoms and contains 1 to 3 heteroatoms each independently selected from N, O and S, the heteroaryl being optionally substituted with one to three substituents each independently selected from the group consisting of halo, C₁ to C₆ alkyl, C₁ to C₆ haloalkyl and C₁ to C₆ alkoxy;
wherein R^{y} is H or C₁ to C₆ alkyl;
   R^{3A} is selected from the group consisting of C₁ to C₆ alkyl optionally wherein the C₁ to C₆ alkyl is substituted with a heterocycloalkyl group; C₃ to C₆ cycloalkyl optionally wherein the C₃ to C₆ cycloalkyl is substituted with one to three substituents each independently selected from the group consisting of halo, C₁ to C₆ alkyl, C₁ to C₆ haloalkyl and C₁ to C₆ alkoxy; phenyl that is optionally substituted with one to three substituents each independently selected from the group consisting of halo, C₁ to C₆ alkyl, C₁ to C₆ haloalkyl and C₁ to C₆ alkoxy; and heteroaryl having 5 to 6 ring atoms containing 1 to 3 heteroatoms each independently selected from N, O and S, the heteroaryl being optionally substituted with one to three substituents each independently selected from the group consisting of halo, C₁ to C₆ alkyl, C₁ to C₆ haloalkyl and C₁ to C₆ alkoxy;
   X¹ is CH₂;
   X² and X³ are each independently CH₂, or a heteroatom selected from O and NR^{x}, wherein R^{x} is H or C₁ to C₆ alkyl, or wherein one R^{1A} group and one R^{x} group combine to form a C₁₋₃ bridge; with the proviso that none or only 1 of X² and X³ is a heteroatom; and
   m is 0, 1, 2 or 3;
   n is 0, 1, 2, or 3; and
   L shows the point of attachment of the linker.

By way of further example, Z may be represented as formula (WId):

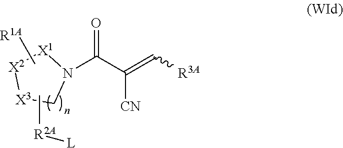

wherein:
R^{1A} is absent or is selected from the group consisting of phenyl that is optionally substituted with one to three substituents selected from the group consisting of halo, C₁ to C₆ alkyl, C₁ to C₆ haloalkyl and C₁ to C₆ alkoxy; heteroaryl having 5 to 6 ring atoms containing 1 to 3 heteroatoms each independently selected from N, O and S, the heteroaryl being optionally substituted with one to three substituents selected from the group consisting of halo, C₁ to C₆ alkyl, C₁ to C₆ haloalkyl and C₁ to C₆ alkoxy; C₃ to C₈ cycloalkyl; C₁ to C₆ alkyl and C₁ to C₆ haloalkyl;
R^{2A} is absent or is selected from the group consisting of phenyl that is optionally substituted with one to three substituents selected from the group consisting of halo, C₁ to C₆ alkyl, C₁ to C₆ haloalkyl and C₁ to C₆ alkoxy; heteroaryl having 5 to 6 ring atoms containing 1 to 3 heteroatoms each independently selected from N, O and S, the heteroaryl being optionally substituted with one to three substituents each independently selected from the group consisting of halo, C₁ to C₆ alkyl, C₁ to C₆ haloalkyl and C₁ to C₆ alkoxy; heterocycloalkyl having 5 to 7 ring atoms and containing 1 to 3 heteroatoms each independently selected from N, O and S, the heterocycloalkyl being optionally substituted with one to three substituents each independently selected from the group consisting of halo, C₁ to C₆ alkyl, C₁ to C₆ haloalkyl and C₁ to C₆ alkoxy; —NR^{y}; —CH(phenyl)-, wherein the phenyl is optionally substituted with one to three substituents each independently selected from the group consisting of halo, C₁ to C₆ alkyl, C₁ to C₆ haloalkyl and C₁ to C₆ alkoxy; and —CH(heteroaryl), wherein the heteroaryl has 5 to 6 ring atoms and contains 1 to 3 heteroatoms each independently selected from N, O and S, the heteroaryl being optionally substituted with one to three substituents each independently selected from the group consisting of halo, C₁ to C₆ alkyl, C₁ to C₆ haloalkyl and C₁ to C₆ alkoxy;
wherein R^{y} is H or C₁ to C₆ alkyl;
R^{3A} is selected from the group consisting of C₁ to C₆ alkyl optionally wherein the C₁ to C₆ alkyl is substituted with a heterocycloalkyl group; C₃ to C₈ cycloalkyl optionally substituted with one to three substituents; heterocycloalkyl having 3 to 10 ring atoms and containing 1 to 3 heteroatoms each independently selected from N, O and S, the heterocycloalkyl being optionally substituted with one to three substituents; phenyl that is optionally substituted with one to three substituents each independently selected from the group consisting of halo, C₁ to C₆ alkyl, C₁ to C₆ haloalkyl and C₁ to C₆ alkoxy; and heteroaryl having 5 to 6 ring atoms containing 1 to 3 heteroatoms each independently selected from N, O and S, the heteroaryl being optionally substituted with one to three substituents each independently selected from the group consisting of halo, C₁ to C₆ alkyl, C₁ to C₆ haloalkyl and C₁ to C₆ alkoxy;
X¹ is CH₂;
X² and X³ are each independently CH₂, or a heteroatom selected from O and NR^{x}, wherein R^{x} is H or C₁ to C₆ alkyl; with the proviso that none or only 1 of X² and X³ is a heteroatom; and
n is 0, 1, 2, or 3; and
L shows the point of attachment of the linker.

By way of further example, Z may be represented as formula (WIe′):

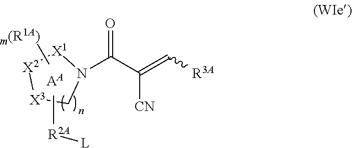

wherein:
R^{1A} is absent (i.e. when m is 0) or is selected from the group consisting of: phenyl; heteroaryl having 5 to 6 ring atoms containing 1 or 2 heteroatoms each independently selected from N, O and S; C₃ to C₇ cycloalkyl; heterocycloalkyl having 5 to 7 ring atoms and containing 1 or 2 heteroatoms each independently selected from N, O and S; C₁ to C₆ alkyl and C₁ to C₆ haloalkyl; wherein the phenyl or heteroaryl is optionally substituted with one substituent selected from the group consisting of halo, C₁ to C₃ alkyl, C₁ to C₃ haloalkyl and C₁ to C₃ alkoxy; and/or wherein two R^{1A} groups combine to form a C₁₋₃ bridge, C₃₋₅cycloalkyl or 5- to 7-membered N-heterocycloalkyl (e.g. wherein the C₃₋₅cycloalkyl or the 5- to 7-membered N-heterocycloalkyl are joined to ring A^{A} at a spiro centre);
R^{2A} is absent or is selected from the group consisting of phenyl; heteroaryl having 5 to 6 ring atoms and containing 1 or 2 heteroatoms each independently selected from N, O and S; heterocycloalkyl having 5 to 7 ring atoms and containing 1 or 2 heteroatoms each independently selected from N, O and S; —NR^{y}; —CH(phenyl)-; and —CH(heteroaryl) wherein the heteroaryl has 5 to 6 ring atoms and contains 1 or 2 heteroatoms each independently selected from N, O and S; and further wherein the phenyl, heteroaryl, heterocycloalkyl, —CH(phenyl)- and —CH(heteroaryl) are each optionally substituted with one substituent selected from the group consisting of halo, C₁ to C₃ alkyl, C₁ to C₃ haloalkyl and C₁ to C₃ alkoxy;
wherein R^{y} is H or C₁ to C₆ alkyl;
R^{3A} is selected from the group consisting of C₁ to C₆ alkyl optionally wherein the C₁ to C₆ alkyl is substituted with a heterocycloalkyl group the heterocycloalkyl having 5 to 7 ring atoms and containing 1 or 2 heteroatoms each independently selected from N, O and S; C₃ to C₆ cycloalkyl; phenyl; and heteroaryl having 5 to 6 ring atoms containing 1 to 3 heteroatoms each independently selected from N, O and S; wherein the C₃ to C₆ cycloalkyl, phenyl and heteroaryl are optionally substituted with one or two substituents selected from the group consisting of halo, C₁ to C₃ alkyl, C₁ to C₃ haloalkyl and C₁ to C₃ alkoxy;
X¹ is CH₂;
X² and X³ are each independently CH₂ or O; with the proviso that none or only 1 of X² and X³ is O;
m is 0, 1, 2 or 3;
n is 1, 2, or 3; and
L shows the point of attachment of the linker.

By way of further example, Z may be represented as formula (WIe):

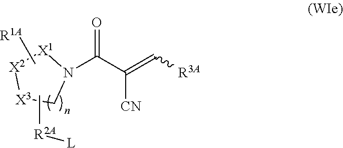

wherein:
R^{1A} is absent or is selected from the group consisting of phenyl; heteroaryl having 5 to 6 ring atoms containing 1 or 2 heteroatoms each independently selected from N, O and S; C₃ to C₇ cycloalkyl; C₁ to C₆ alkyl and C₁ to C₆ haloalkyl; wherein the phenyl or heteroaryl is optionally substituted with one substituent selected from the group consisting of halo, C₁ to C₃ alkyl, C₁ to C₃ haloalkyl and C₁ to C₃ alkoxy;
R^{2A} is absent or is selected from the group consisting of phenyl; heteroaryl having 5 to 6 ring atoms and containing 1 or 2 heteroatoms each independently selected from N, O and S; heterocycloalkyl having 5 to 7 ring atoms and containing 1 or 2 heteroatoms each independently selected from N, O and S; —NR^{y}; —CH(phenyl)-; and —CH(heteroaryl) wherein the heteroaryl has 5 to 6 ring atoms and contains 1 or 2 heteroatoms each independently selected from N, O and S; and further wherein the phenyl, heteroaryl, heterocycloalkyl, —CH(phenyl)- and —CH(heteroaryl) are each optionally substituted with one substituent selected from the group consisting of halo, C₁ to C₃ alkyl, C₁ to C₃ haloalkyl and C₁ to C₃ alkoxy;
wherein R^{y} is H or C₁ to C₆ alkyl;
R^{3A} is selected from the group consisting of C₁ to C₆ alkyl optionally wherein the C₁ to C₆ alkyl is substituted with a heterocycloalkyl group the heterocycloalkyl having 5 to 7 ring atoms and containing 1 or 2 heteroatoms each independently selected from N, O and S; C₃ to C₆ cycloalkyl; phenyl; and heteroaryl having 5 to 6 ring atoms containing 1 to 3 heteroatoms each independently selected from N, O and S; wherein the C₃ to C₆ cycloalkyl, phenyl and heteroaryl are optionally substituted with one or two substituents selected from the group consisting of halo, C₁ to C₃ alkyl, C₁ to C₃ haloalkyl and C₁ to C₃ alkoxy;
X¹ is CH₂;
X² and X³ are each independently CH₂ or O; with the proviso that none or only 1 of X² and X³ is O; and
n is 1, 2, or 3; and
L shows the point of attachment of the linker.

In further embodiments, Z comprises a structure according to formula (WZII):

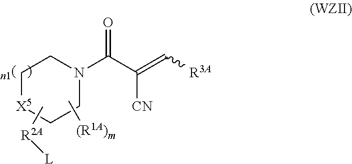

wherein R^{2A} is absent or is as described in any one of the embodiments disclosed herein;
R^{3A} is as described in any one of the embodiments disclosed herein;
X⁵ is CR^{b}₂, NR^{b}, O or a 5- to 7-membered heterocycloalkyl (e.g. a 5- to 7-membered heterocycloalkyl);
each R^{1A} is independently selected from aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R^{1A} groups combine to form an optionally substituted C₁₋₃ bridge or optionally substituted C₃₋₅cycloalkyl (optionally wherein the C₃₋₅cycloalkyl is joined to the heterocyclic ring shown in formula (WZII) at a spiro centre);
R^{b} is H or optionally substituted C₁₋₃alkyl;
n1 is 0, 1, 2 or 3;
m is 0, 1 or 2; and
L shows the point of attachment of the linker.

In yet further embodiments, Z comprises a structure according to any one of formulae (WZIIa) to (WZIIe):

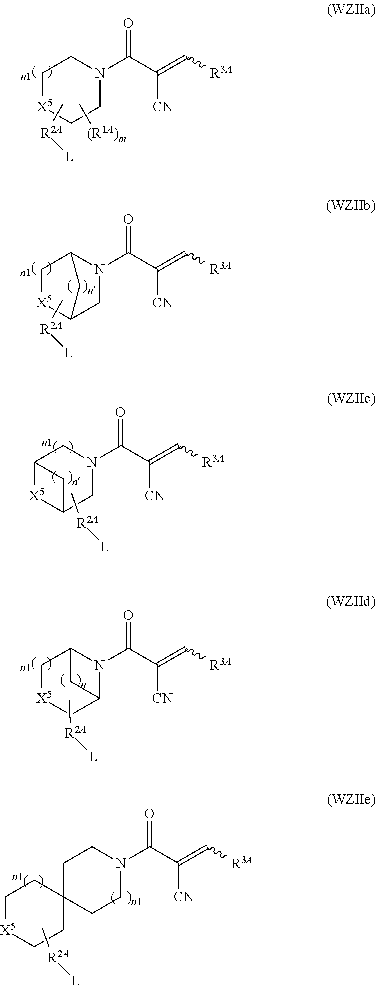

wherein:
R^{2A} is as described in any one of the embodiments disclosed herein;
R^{3A} is as described in any one of the embodiments disclosed herein;
each R^{1A} is independently selected from aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R^{1A} groups combine to form an optionally substituted C₃₋₅cycloalkyl (optionally wherein the C₃₋₅cycloalkyl is joined to the heterocyclic ring shown in formula (ZIIa) at a spiro centre);
X⁵ is C(R^{b})₂, NR^{b} or O;
R^{b} is H or optionally substituted C₁₋₃alkyl;
n1 is 0, 1, 2 or 3;
n′ is 1 or 2;
m is 0, 1 or 2; and
L shows the point of attachment of the linker.

For example, Z may comprise a structure according to formula (WZIIIa) to (WZIIIh):

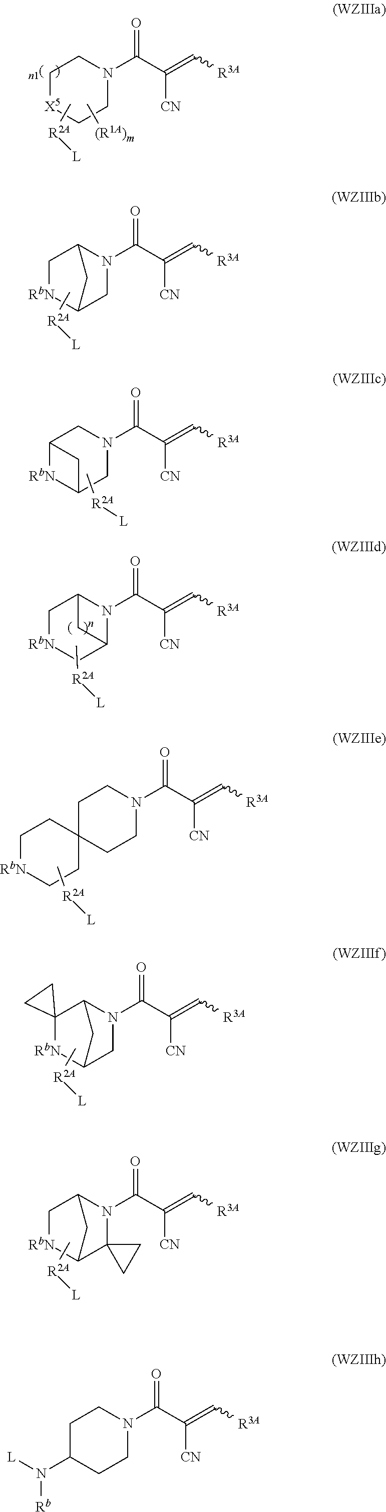

wherein:
R^{2A} is as described in any one of the embodiments disclosed herein;
R^{3A} is as described in any one of the embodiments disclosed herein;
each R^{1A} is independently selected from aryl, substituted aryl, heteroaryl, substituted heteroaryl, cycloalkyl, substituted cycloalkyl, C₁ to C₆ alkyl and substituted C₁ to C₆ alkyl;
X⁵ is CH₂, NR^{b} or O;
R^{b} is H or optionally substituted C₁₋₃alkyl;
n1 is 0, 1 or 2;
n′ is 1 or 2;
m is 0, 1 or 2; and
L shows the point of attachment of the linker.

In even further embodiments, Z comprises a structure according to formula (WZIVa) to (WZIVj):

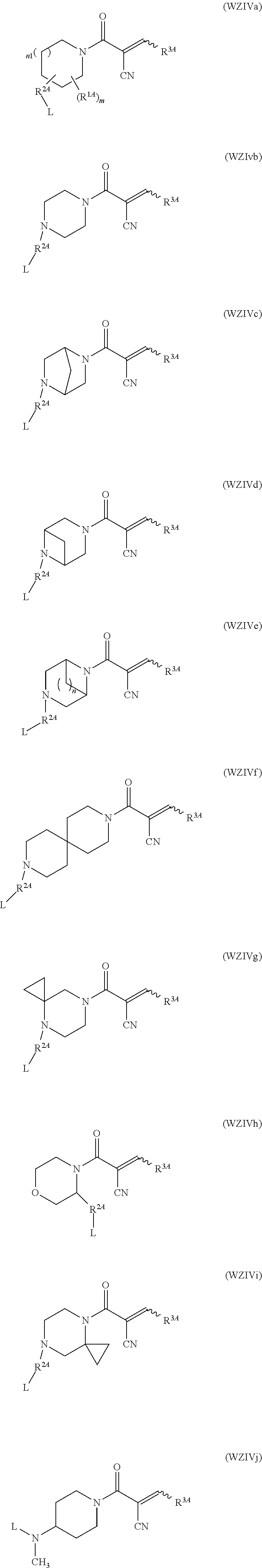

wherein:
R^{2A} is absent or is as described in any one of the embodiments disclosed herein;
R^{3A} is as described in any one of the embodiments disclosed herein;
each R^{1A} is independently selected from aryl, substituted aryl, heteroaryl, substituted heteroaryl, cycloalkyl, substituted cycloalkyl, C₁ to C₆ alkyl and substituted C₁ to C₆ alkyl;
n1 is 0, 1 or 2;
n′ is 1 or 2;
m is 0, 1 or 2; and
L shows the point of attachment of the linker.

In further examples, Z comprises a structure according to formula (WIf):

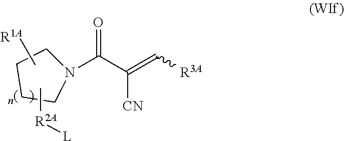

wherein R^{1A} is absent or is selected from aryl, substituted aryl, heteroaryl, substituted heteroaryl, cycloalkyl, C₁ to C₆ alkyl and substituted C₁ to C₆ alkyl;
R^{2A} is absent or is selected from aryl, substituted aryl, heteroaryl, substituted heteroaryl, heterocycloalkyl, substituted heterocycloalkyl, —CH(aryl)- and —CH(substituted aryl)-;
R^{3A} is selected from C₁ to C₆ alkyl, aryl, heteroaryl, substituted C₁ to C₆ alkyl, substituted aryl, and substituted heteroaryl; and
wherein at least one of R^{1A} and R^{2A} is present;
n is 0, 1, 2, or 3; and
L shows the point of attachment of the linker.

In some examples, R^{1A}, R^{2A} and R^{3A} of formula (WIf) may be selected from those groups defined above for any one or more of formulae (WIc′), (WIc), (WId′), (WId), (WIe′) or (WIe).

In some examples of formulae (WZI), (WI) and the various subgeneric formula described above and herein, n may be 1, 2 or 3 and/or n1 may be 0, 1 or 2.

In those cases where R^{1A} is absent, Z may be represented by formula (WI):

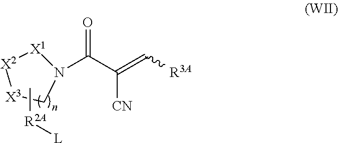

wherein R^{2A} is selected from aryl, substituted aryl, heteroaryl, substituted heteroaryl, heterocycloalkyl, substituted heterocycloalkyl, —CH(aryl)-, —CH(substituted aryl)-, —CH(heteroaryl)- and —CH(substituted heteroaryl);
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group;
X¹ is CH₂;
X² and X³ are each independently CH₂ or O; with the proviso that none or only 1 of X² and X³ is O; and
n is 0, 1, 2 or 3; and
L shows the point of attachment of the linker.

In those cases where R^{1A} is absent, Z may be represented by formula (WIIa):

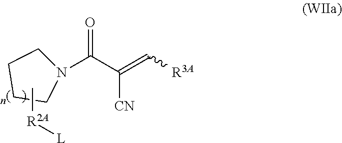

wherein R^{2A} is selected from aryl, substituted aryl, heteroaryl, substituted heteroaryl, heterocycloalkyl, substituted heterocycloalkyl, —CH(aryl)-, —CH(substituted aryl)-, —CH(heteroaryl)- and —CH(substituted heteroaryl);
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group; and
n is 0, 1, 2 or 3; and
L shows the point of attachment of the linker.

By way of particular example, in formulae (WII) or (WIIa), n may be 1 or 2.

By way of further example, Z may be represented by formula (WIIb):

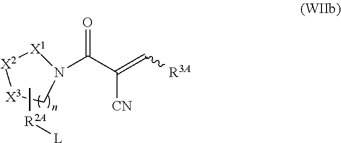

wherein R^{2A} is selected from aryl substituted aryl, heteroaryl, substituted heteroaryl, heterocycloalkyl, and substituted heterocycloalkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group;
X¹ is CH₂;
X² and X³ are each independently CH₂ or O; with the proviso that none or only 1 of X² and X³ is O;
n is 1 or 2; and
L shows the point of attachment of the linker.

By way of further example, Z may be represented by formula (WIIc):

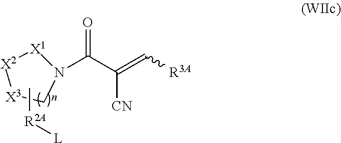

wherein R^{2A} is selected from heterocycloalkyl and substituted heterocycloalkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group;
X¹ is CH₂;
X² and X³ are each independently CH₂ or O; with the proviso that none or only 1 of X² and X³ is O;
n is 1 or 2; and
L shows the point of attachment of the linker.

In some cases, Z may be represented by formula (WIId):

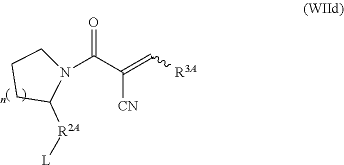

wherein R^{2A} is selected from heterocycloalkyl and substituted heterocycloalkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group;
n is 1 or 2; and
L shows the point of attachment of the linker.

In other examples, Z may comprise a structure according to formula (WIIe):

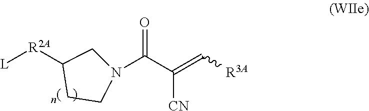

wherein R^{2A} is selected from aryl, substituted aryl, heteroaryl, substituted heteroaryl, heterocycloalkyl and substituted heterocycloalkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group;
n is 1 or 2; and
L shows the point of attachment of the linker.

In other examples, Z may comprise a structure according to formula (WIIf):

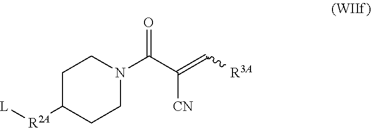

wherein R^{2A} is selected from aryl, substituted aryl, heteroaryl, substituted heteroaryl, heterocycloalkyl and substituted heterocycloalkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group; and
L shows the point of attachment of the linker.

In those cases where R^{2A} is absent, Z may comprise a structure according to formula (WIII):

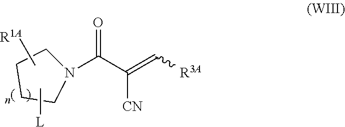

wherein R^{1A} is selected from aryl, substituted aryl, heteroaryl, substituted heteroaryl, cycloalkyl and C₁ to C₆ alkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group; and
n is 0, 1, 2 or 3; and
L shows the point of attachment of the linker.

In some examples, n may be 1 or 2.

In some examples where n is 2, Z may be represented by formula (WIIIa):

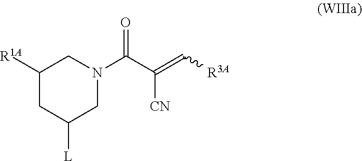

wherein R^{1A} is selected from aryl, substituted aryl, heteroaryl, substituted heteroaryl, cycloalkyl and C₁ to C₆ alkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group; and
L shows the point of attachment of the linker.

In some examples where n is 1, Z may be represented by formula (WIIIb):

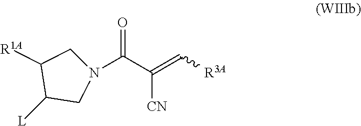

wherein R^{1A} is selected from aryl, substituted aryl, heteroaryl, substituted heteroaryl, cycloalkyl and C₁-C₆ alkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group; and
L shows the point of attachment of the linker.

As illustrated above, bifunctional molecules of formula (WIIIb) comprise at least two stereocentres and so exist in several diastereomeric (and enantiomeric) forms. In some examples, the groups R^{1A} and L may exist in a trans relationship (e.g. these groups are held and/or oriented on opposite sides of the heterocyclic core). In other examples, the groups R^{1A} and L may exist in a cis relationship (e.g. these groups are held and/or oriented on the same side of the heterocyclic core). By way of further example, bifunctional molecules of formula (WIIIb) may encompass at least the following diastereomeric forms:

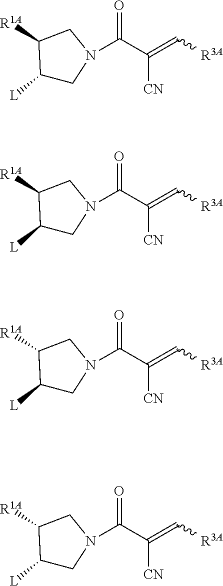

In those examples where R^{1A} is absent and R^{2A} is selected from CH(aryl)-, —CH(substituted aryl)-, —CH(heteroaryl)- and —CH(substituted heteroaryl)-, Z may be represented by formula (WIV):

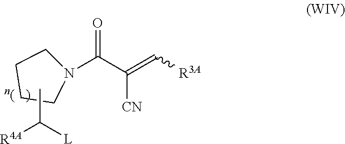

wherein R^{3A} is selected from C₁ to C₆ alkyl, aryl, heteroaryl, substituted aryl, and substituted heteroaryl, optionally wherein the C₁ to C₆ alkyl is substituted with a heterocycloalkyl group;
R^{4A} is selected from aryl, substituted aryl, heteroaryl and substituted heteroaryl; and
n is 0, 1, 2 or 3; and
L shows the point of attachment of the linker.

In some examples, Z may comprise a structure according to formula (WIVa):

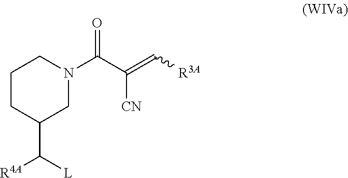

wherein R^{3A} is selected from C₁ to C₆ alkyl, aryl, heteroaryl, substituted aryl, and substituted heteroaryl, optionally wherein the C₁ to C₆ alkyl is substituted with a heterocycloalkyl group;
R^{4A} is selected from aryl, substituted aryl, heteroaryl and substituted heteroaryl; and
L shows the point of attachment of the linker.

In either of formula (WIV) or (WIVa), R^{4A} may be selected from aryl or substituted aryl.

With respect to the various structures for Z defined by the formulae (WI) to (WIV) (and subgeneric formulae thereof) herein (and unless otherwise stated), R^{1A} may be selected from aryl, substituted aryl, heteroaryl, substituted heteroaryl, cycloalkyl, substituted cycloalkyl, C₁ to C₆ alkyl, and substituted C₁ to C₆ alkyl.

In some examples, R^{1A} is an optionally substituted aryl or an optionally substituted heteroaryl.

Where R^{1A} is a substituted aryl or substituted heteroaryl, the aryl or heteroaryl may comprise one or more substituents selected from the group consisting of C₁ to C₆ alkyl (e.g. methyl), C₁ to C₆ alkoxy (e.g. methoxy), C₁ to C₆ haloalkyl and halo.

By way of further example, R^{1A} may be phenyl that is optionally substituted with one to three substituents selected from the group consisting of halo, C₁ to C₆ alkyl, C₁ to C₆ haloalkyl and C₁ to C₆ alkoxy. By way of a yet further example, R^{1A} may be heteroaryl having 5 to 6 ring atoms containing 1 to 3 heteroatoms each independently selected from N, O and S, the heteroaryl being optionally substituted with one to three substituents selected from the group consisting of halo, C₁ to C₆ alkyl, C₁ to C₆ haloalkyl and C₁ to C₆ alkoxy; C₃ to C₈ cycloalkyl.

Representative examples of suitable R^{1A} groups include but are not limited to phenyl, substituted phenyl, pyrazolyl, and substituted pyrazolyl.

In some examples, R^{1A} is a cycloalkyl, such as a C₃ to C₇ cycloalkyl, or a C₃ to C₆ cycloalkyl.

In some examples, R^{1A} is a C₁ to C₆ alkyl, such as a C₁ to C₃ alkyl that is optionally substituted with one to three substituents as defined herein.

Further non-limiting examples of suitable R^{1A} groups are illustrated below:

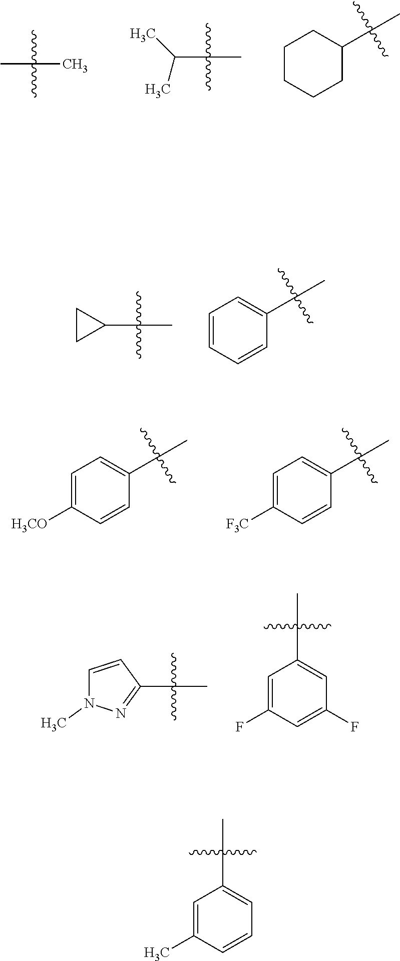

Further non-limiting examples of suitable R^{1A} groups are:

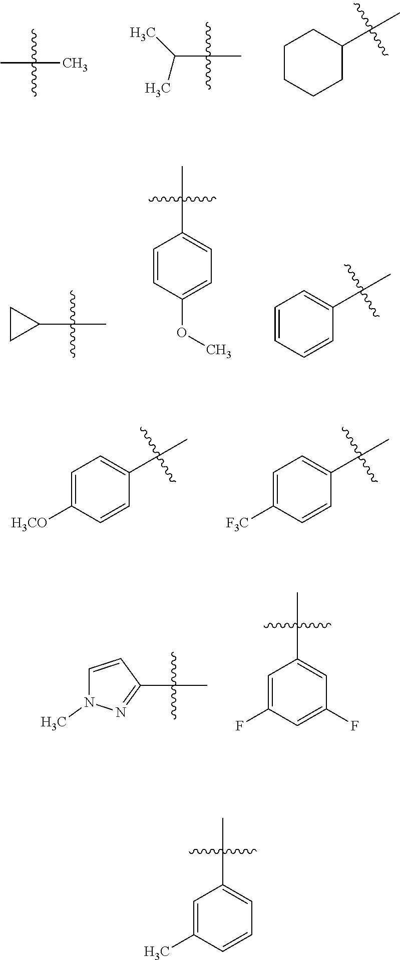

Further non-limiting examples of suitable R^{1A} groups are:

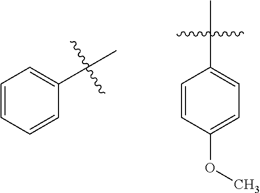

In the structures shown above, the line intersected by a wavy line represents the covalent bond between the exemplary R^{1A} groups shown above and a carbon atom on the heterocycloalkyl core attached to the R^{1A} group in the parent structure of Z (as illustrated by the various formulae (WI) to (WIV) (and sub-generic formulae) described herein). Although a particular substitution pattern is shown in the exemplary aryl and heteroaryl structures above, it will be appreciated that other substitution patterns are also encompassed within the scope of the present disclosure.

In further examples, such as in respect of formulae (WZII), two R^{1A} groups may combine to form a C₁₋₃ bridge or C₃₋₅cycloalkyl. For example, two R^{1A} groups may combine to form a C₃₋₅cycloalkyl. In such examples, the C₃₋₅cycloalkyl may be joined to the heterocyclic ring of the parent structure at a spiro centre.

With respect to the various structures for Z defined by the formulae (WZI) to (WZIV), (WI) to (WIV) (and sub-generic formulae) described herein (and unless otherwise stated), R^{2A} may be selected from aryl, substituted aryl, heteroaryl, substituted heteroaryl, heterocycloalkyl, substituted heterocycloalkyl, NR^{y}, —CH(aryl)-, —CH(substituted aryl)-, —CH(heteroaryl) and —CH(substituted heteroaryl); wherein R^{y} is optionally substituted C₁₋₆alkyl (such as methyl) or H.

In some examples, R^{2A} is present in Z (and/or the bifunctional molecules described herein) as a divalent group. In other words, as shown in formulae (WI) to (WIVa) (and unless otherwise stated), the various groups defined for R^{2A} are covalently attached to an atom of the heterocyclic core of Z and also may be covalently attached to an atom of a linker. Thus, these groups may be considered as divalent radical species.

Where R^{2A} is selected from optionally substituted aryl and optionally substituted heteroaryl, R^{2A} may be selected from aryl having 6 to 10 carbon ring atoms, the aryl being optionally substituted with one to three substituents; and heteroaryl having 5 to 10 ring atoms and containing 1 to 3 heteroatoms each independently selected from N, O and S, the heteroaryl being optionally substituted with one to three substituents. By way of further example, R^{2A} may be selected from phenyl optionally substituted with one to three substituents selected from H, C₁ to C₆ alkyl, halo, C₁ to C₆ haloalkyl and C₁ to C₆ alkoxy; and heteroaryl having 5 to 6 ring atoms and containing 1 or 2 N atoms, the heteroaryl being optionally substituted with one to three substituents selected from C₁-C₆ alkyl (e.g. C₁ to C₃ alkyl), halo (e.g. F), C₁-C₆ haloalkyl (e.g. C₁ to C₃ haloalkyl) and C₁ to C₆ alkoxy (e.g. C₁ to C₃ alkoxy). In some cases, suitable examples of R^{2A} include (but are not limited to) optionally substituted phenyl, and optionally substituted pyrazolyl.

Where R^{2A} is selected from optionally substituted heterocycloalkyl, the heterocycloalkyl may have 3 to 10 ring atoms and contain 1 to 3 heteroatoms each independently selected from N, O and S, and the heterocycloalkyl may be optionally substituted with one to three substituents. In some examples, the heterocycloalkyl may have 5 to 8 ring atoms (e.g. 6 ring atoms) and may contain 1 or 2 N atoms. In some cases, suitable examples include (but are not limited to) optionally substituted piperidinyl, and optionally substituted piperazinyl.

Further examples of suitable R^{2A} groups are shown below:

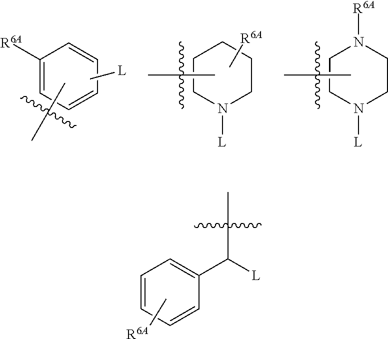

wherein in the structures shown above, R^{6A} may be selected from H, C₁-C₆ alkyl, halo, C₁-C₆ haloalkyl and C₁-C₆ alkoxy. In some examples, R^{6A} may be selected from H and C₁-C₆ alkyl.

Further examples of suitable R^{2A} groups are shown below:

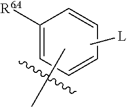

wherein R^{6A} is selected from H, C₁-C₆ alkyl, halo, C₁-C₆ haloalkyl and C₁-C₆ alkoxy. In some examples, R^{6A} may be selected from H and C₁-C₆ alkyl.

In the structures shown above, the line intersected by a wavy line represents the covalent bond between the exemplary R^{2A} groups shown above and a carbon atom on the heterocycloalkyl core attached to the R^{2A} group in the parent structure of Z (as illustrated by the various formulae (WI) to (WIV) (and sub-generic formulae thereof) described herein and unless otherwise stated). Although a particular substitution pattern is shown in the exemplary structures above, it will be appreciated that other substitution patterns are also encompassed within the scope of the present disclosure.

In addition, the bond to L shows the point of attachment to the linker. In the exemplary aryl structure above, it will be appreciated that the linker may replace a hydrogen atom at any suitable position on the aryl ring (e.g. provided it is chemically suitable and has the correct valency).

With respect to the various structures for Z defined by the various formulae (WI) to (WIV) (and sub-generic formulae thereof) described herein, R^{3A} is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S. In some examples, R^{3A} is selected from C₁ to C₆ alkyl, aryl, heteroaryl, substituted C₁ to C₆ alkyl, substituted aryl, and substituted heteroaryl.

In some examples, R^{3A} may be selected from the group consisting of C₁ to C₆ alkyl optionally substituted with a heterocycloalkyl group having 5 to 7 ring atoms and containing 1 or 2 heteroatoms each independently selected from N, O and S; aryl having 6 to 10 carbon ring atoms; and heteroaryl having 5 to 10 ring atoms and containing 1 to 3 heteroatoms each independently selected from N, O and S; wherein the aryl and the heteroaryl are optionally substituted with one or two substituents selected from the group consisting of halo, C₁ to C₃ alkyl, C₁ to C₃ haloalkyl and C₁ to C₃ alkoxy. By way of further example, in some cases the aryl and heteroaryl may be optionally substituted with one or two substituents selected from halo (e.g. F) and C₁ to C₃ alkyl (e.g. methyl).

Representative examples of suitable R^{3A} groups include, but are not limited to, thiazolyl, pyridinyl, benzothiazolyl, phenyl, pyrazolyl, isoxazolyl, isothiazolyl, oxetanyl, cyclobutanyl, cyclopropanyl, tert-butyl, imidazolyl, oxazolyl, thiophenyl, imidazo(1,2-a)pyridinyl, N—C₁ to C₆ alkylenemorpholine, and 4,5,6,7-tetrahydro-1,3-benzothiazolyl, such as thiazolyl, pyridinyl, benzothiazolyl, phenyl, pyrazolyl, isoxazolyl, isothiazolyl, tetrahydropyranyl, tetrahydrofuranyl, oxetanyl, cyclobutanyl, cyclopropanyl and tert-butyl.

In each case, these R^{3A} groups may be substituted, such as substituted thiazolyl, substituted pyridinyl, substituted benzothiazolyl, substituted phenyl, substituted pyrazolyl, substituted isoxazolyl, substituted isothiazolyl, substituted tetrahydropyranyl, substituted tetrahydrofuranyl, substituted oxetanyl, substituted cyclobutanyl, substituted cyclopropanyl and substituted tert-butyl. Where R^{3A} is a substituted heteroaryl or aryl group, there may be one or more substituents on the aromatic ring e.g. it may be mono-, di- or tri-substituted. Where R^{3A} is optionally substituted pyrazolyl or imidazolyl, a nitrogen atom of the pyrazolyl or imidazolyl ring may be substituted with C₁ to C₆ alkyl, such as methyl.

Representative examples of suitable R^{3A} groups include, but are not limited to, optionally substituted phenyl, optionally substituted thiazolyl, optionally substituted pyrazolyl, optionally substituted oxazoyl, optionally substituted isoxazolyl, tert-butyl, C₁-C₆ alkyl comprising a morpholino substituent, optionally substituted benzothiazolyl and optionally substituted pyridinyl. Where R^{3A} is a substituted aryl or heteroaryl group, there may be one or more substituents on the aromatic ring e.g. it may be mono-, di- or tri-substituted.

Representative examples of suitable R^{3A} groups include, but are not limited to, optionally substituted phenyl, optionally substituted thiazolyl, optionally substituted pyrazolyl, optionally substituted oxazoyl, tert-butyl, C₁-C₆ alkyl comprising a morpholino substituent, optionally substituted benzothiazolyl and optionally substituted pyridinyl.

Further examples of suitable R^{3A} groups are shown below:

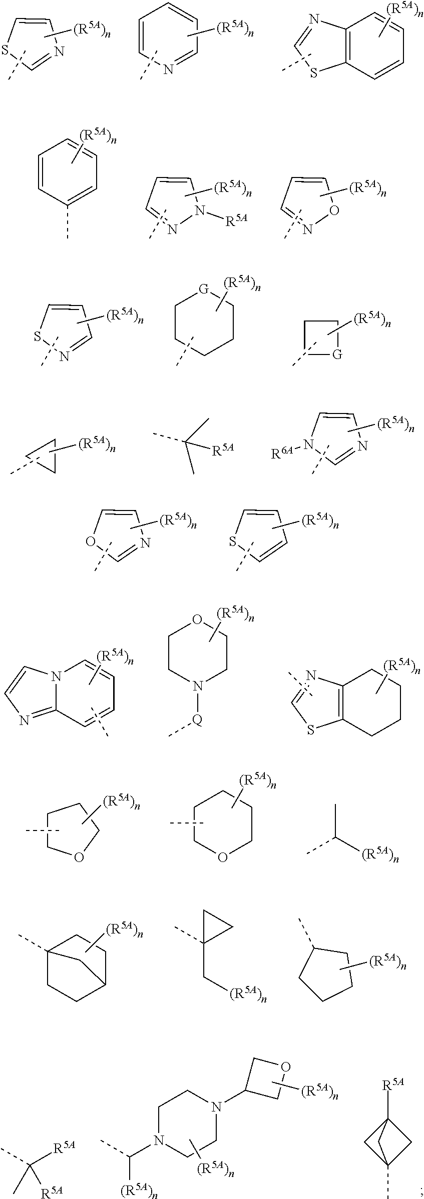

wherein the dotted line on the structures indicates the position that each of the respective R^{3A} groups may be joined to the structure shown in the formulae described herein. Where the dotted line is not shown connected directly to an atom, the R^{3A} group may be connected to the structure shown in formulae by a covalent bond to an atom at any position on the aromatic ring (provided that it has the correct valency and/or is chemically suitable). For example, a hydrogen at any position on the R^{3A} group may be replaced with a bond to the parent structures as shown in the formulae described herein.

R^{5A} may be any substituent as described herein or may be absent. In some examples, R^{5A} may be selected from halo (e.g. F, Cl, Br, I), CF₃, —CH₂F, —CHF₂, OCF₃, —OCH₂F, —OCHF₂, C₁ to C₆ alkyl, —CN, —OH, —OMe, —SMe, —SOMe, —SO₂Me, —NH₂, —NHMe, —NMe₂, CO₂Me, —NO₂, CHO, and COMe. As stated above, there may be one or more substituents on the aromatic ring (e.g. n may be 0 to 5, such as 0 to 4, 0 to 3, or 0 to 2). Where more than one substituent is present, each substituent may be independently selected from the R^{5A} groups noted above.

R^{6A} may be C₁ to C₆ alkyl, such as methyl.

G may be selected from CH₂, O and NH.

Q may be C₁ to C₆ alkylene such as dimethylmethylene (—C(CH₃)₂—) or dimethylethylene (—C(CH₃)₂CH₂—).

In further embodiments, R³ is selected from the group consisting of

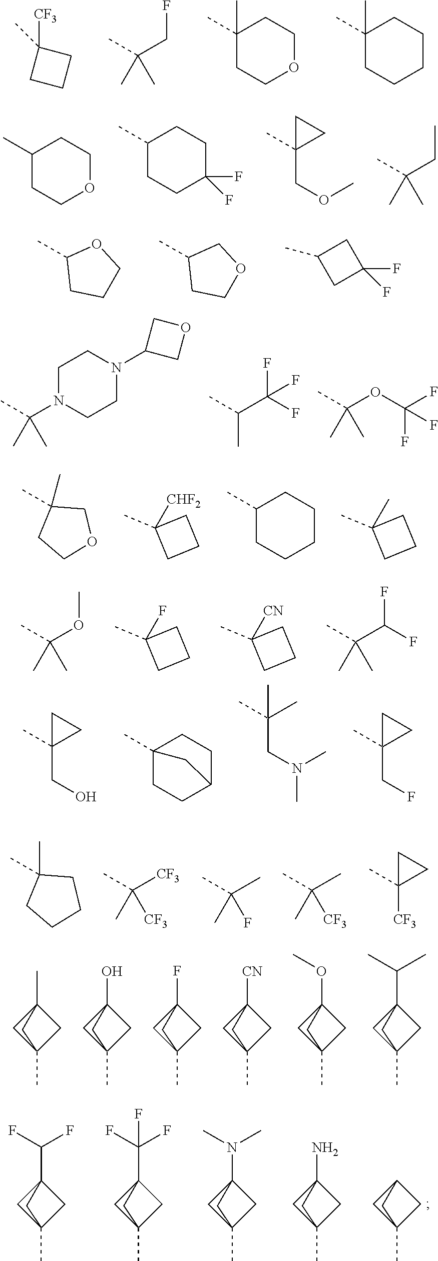

wherein the dotted line indicates the position at which each of the respective R³ groups is joined to the structure in the formulae described herein.

By way of further example, R^{5A} may be selected from C₁ to C₆ alkyl (e.g. methyl) and halo (e.g. F). As stated above, there may be one or more substituents on the aromatic ring. Where two or more substituents are present, each substituent may be independently selected from the R^{5A} groups noted above. Again, where present and unless otherwise indicated, R^{5A} may be appended to the aryl or heteroaryl ring at any position (provided that it has the correct valency and/or is chemically suitable).

In the structures shown above, the line intersected by a wavy line represents the covalent bond between the exemplary R^{3A} groups shown above and the carbon atom of the parent structure of Z (as illustrated by the various formulae (WZI) to (WZV), (WI) to (WIV) (and sub-generic formulae thereof) described herein). In those cases where R^{3A} is an aryl or heteroaryl group, this covalent bond (as illustrated in the various formulae described herein) may be formed at any position on the aromatic ring (provided that it has the correct valency and/or is chemically suitable). For example, a hydrogen at any position on the R^{3A} groups shown above may be replaced with a bond to the structure shown in formula (I).

By way of further example, a suitable R^{3A} group may be selected from the following:

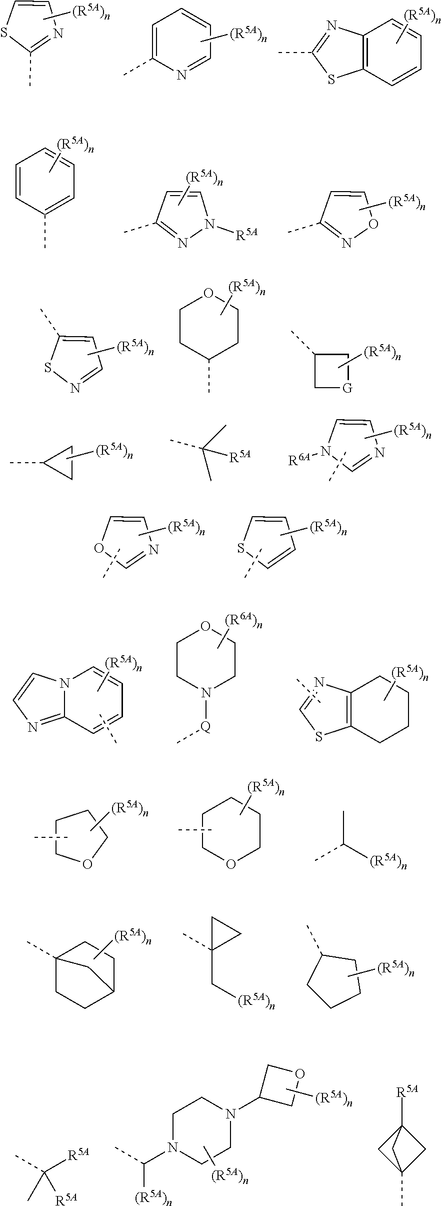

wherein the dotted line on the structures indicates the position that each of the respective R^{3A} groups may be joined to the structure shown in formulae described herein, and R^{5A}, R^{6A}, n and G are as defined above.

In other examples, a suitable R^{3A} group may be selected from the following:

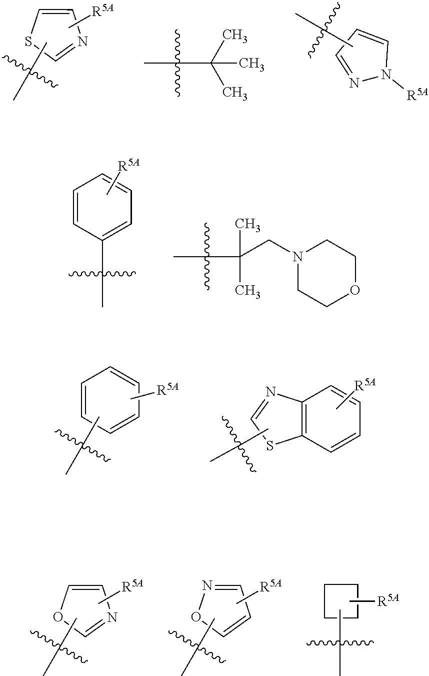

wherein the line intersected by a wavy line represents the covalent bond between the exemplary R^{3A} groups shown above and the carbon atom of the parent structure of Z (as illustrated by the various formulae described herein), and R^{5A} is as defined above.

In other examples, a suitable R^{3A} group may be selected from the following:

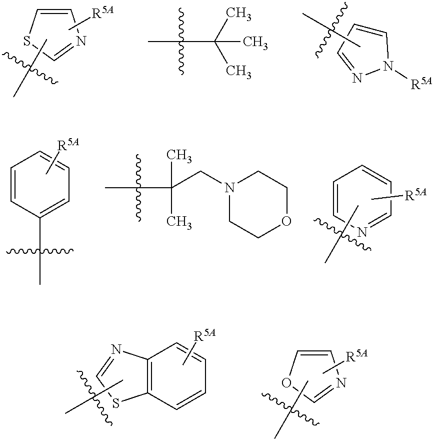

wherein the line intersected by a wavy line represents the covalent bond between the exemplary R^{3A} groups shown above and the carbon atom of the parent structure of Z (as illustrated by the various formulae described herein), and R^{5A} is as defined above.

By way of further examples, a suitable R^{3A} group may be selected from the following:

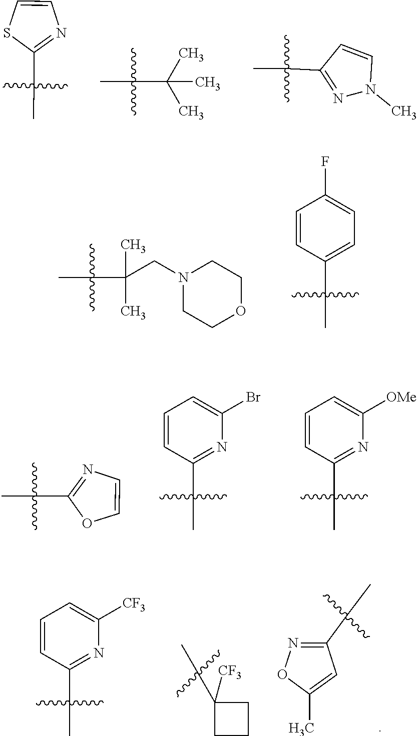

Again, in the structures shown above, the line intersected by a wavy line represents the covalent bond between the exemplary R^{3A} groups shown above and the carbon atom of the parent structure of Z (as illustrated by the various formulae (WZI) to WZV), (WI) to (WIV) (and sub-generic formulae thereof) described herein).

By way of further example, a suitable R^{3A} group may be selected from the following:

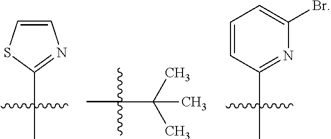

Again, in the structures shown above, the line intersected by a wavy line represents the covalent bond between the exemplary R^{3A} groups shown above and the carbon atom of the parent structure of Z (as illustrated by the various formulae (WZI) to WZV), (WI) to (WIV) (and sub-generic formulae thereof) described herein).

By way of another example, the R^{3A} group may be:

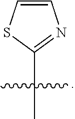 [SMILES: *c1nccs1]

Again, in the structures shown above, the line intersected by a wavy line represents the covalent bond between the exemplary R^{3A} group shown above and the carbon atom of the parent structure of Z (as illustrated by the various formulae (WZI) to WZV), (WI) to (WIV) (and sub-generic formulae thereof) described herein).

As stated above, R^{4A} may be selected from aryl, substituted aryl, heteroaryl and substituted heteroaryl. In some examples, R^{4A} may be selected from aryl having 6 to 10 carbon ring atoms; and heteroaryl having 5 to 10 ring atoms and containing 1 to 3 heteroatoms each independently selected from N, O and S; wherein the aryl and the heteroaryl are optionally substituted with one or two substituents selected from the group consisting of halo, C₁ to C₃ alkyl, C₁ to C₃ haloalkyl and C₁ to C₃ alkoxy. In some examples, R^{4A} may be an optionally substituted phenyl.

By way of further example, a suitable R^{4A} group may be selected from the following:

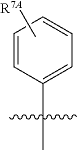

R^{7A} may be any substituent as described herein or may be absent. In some examples, R^{7A} may be selected from C₁ to C₆ alkyl, halo, C₁ to C₆ haloalkyl and C₁ to C₆ alkoxy. In some examples, R^{6A} may be C₁ to C₆ alkyl or C₁ to C₃ alkyl (e.g. methyl). As stated above, there may be one or more substituents on the aromatic ring. Where two or more substituents are present, each substituent may be independently selected from the R^{7A} groups noted above. Again, where present and unless otherwise indicated, R^{7A} may be covalently bonded to the aryl or heteroaryl ring at any position (provided that it has the correct valency and/or is chemically suitable).

By way of further example, representative examples of Z are illustrated below:

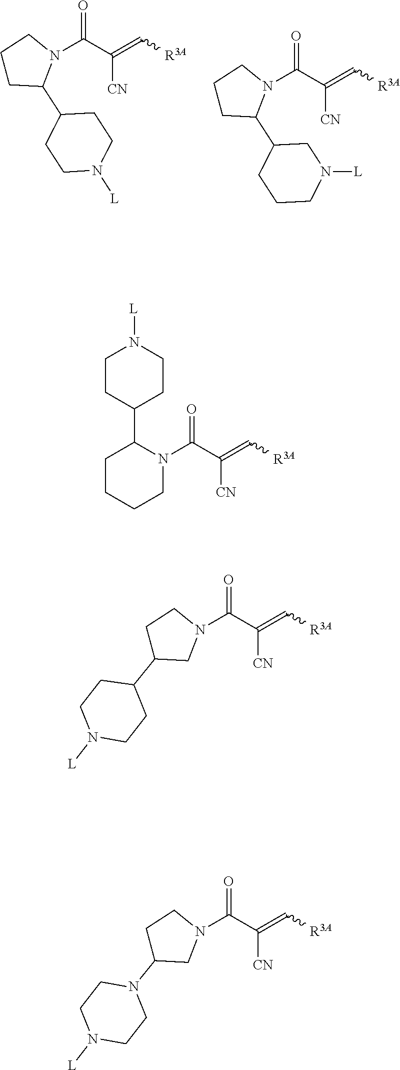

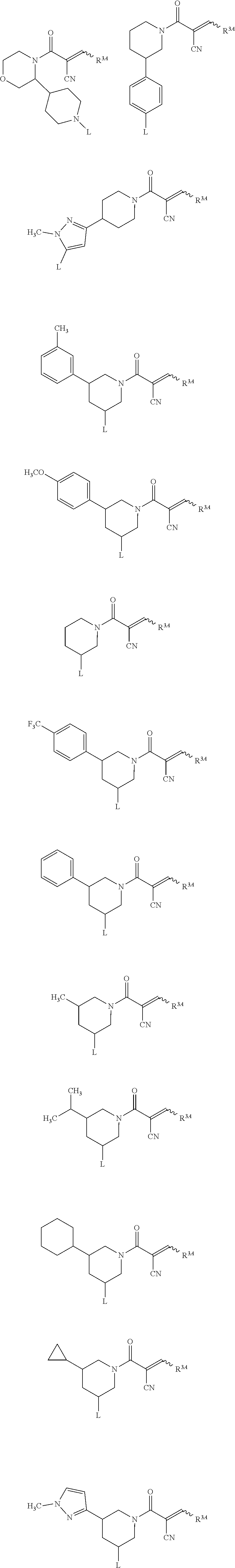

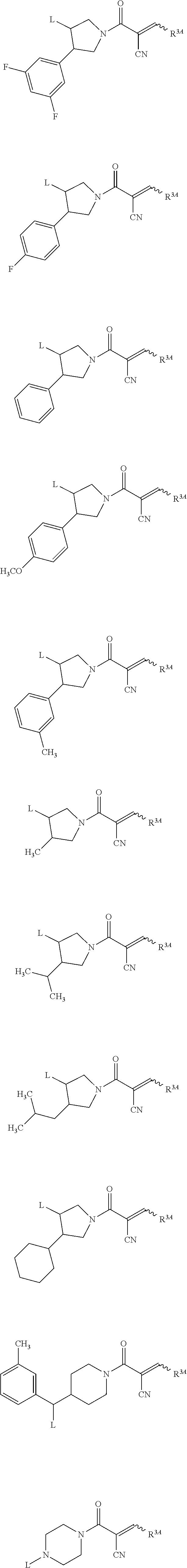

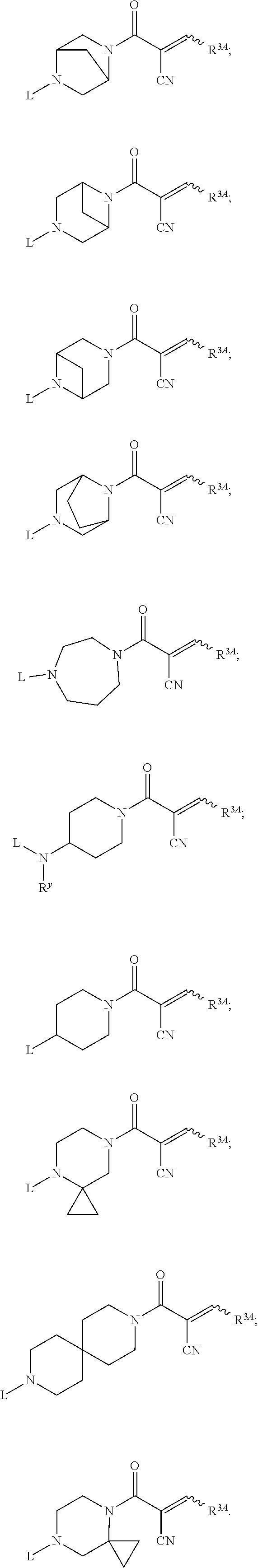

By way of further example, representative examples of Z are illustrated below:

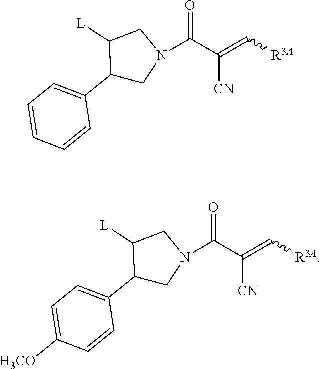

In the exemplary structures shown above, R^{3A} may be selected from any of those R^{3A} groups disclosed herein. In some cases, in the exemplary structures shown above, R^{3A} may be selected from the group consisting of:

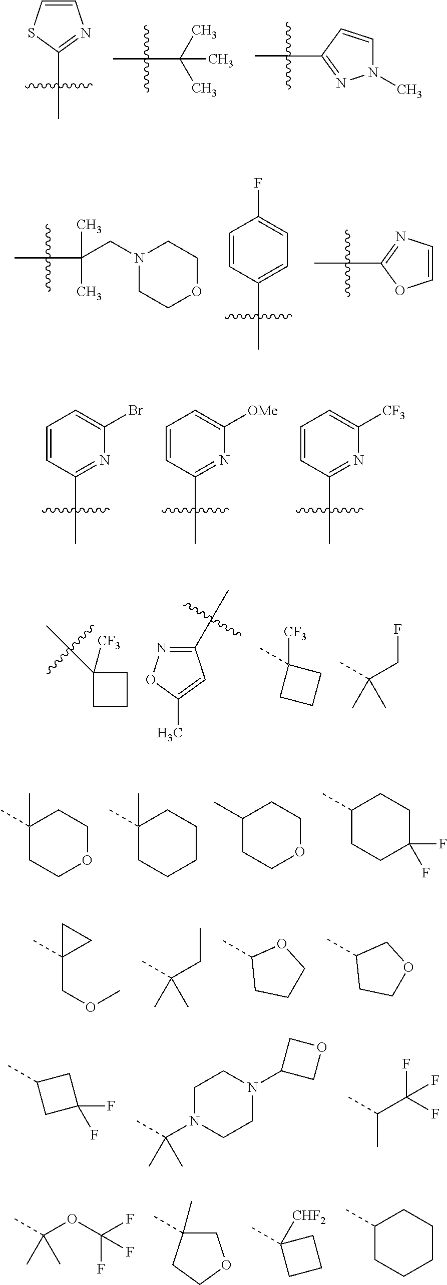

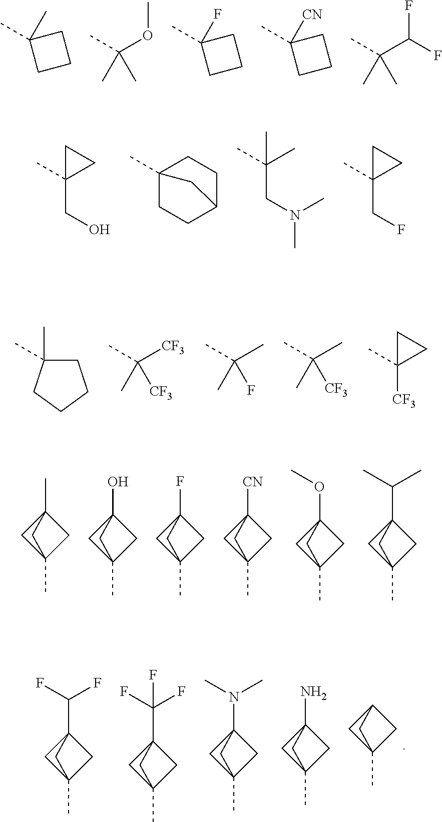

In the exemplary structures shown above, R^{3A} may be selected from any of those R^{3A} groups disclosed herein. In some cases, in the exemplary structures shown above, R^{3A} may be:

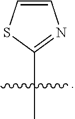 [SMILES: *c1nccs1]

In particular examples, Z is of formula:

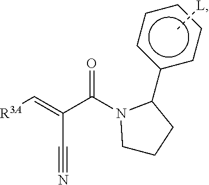

where R^{3A} is as defined above.

For example, Z may be any one of the structures shown below:

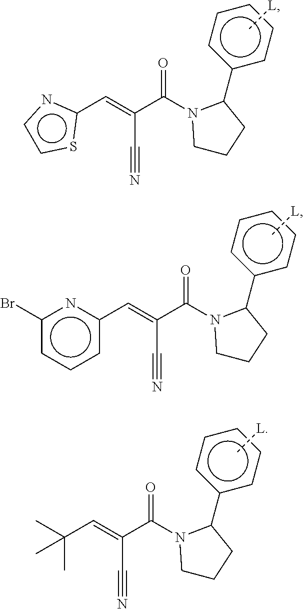

In particular examples, Z is of formula:

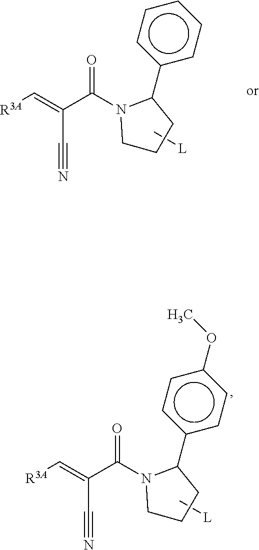

where R^{3A} is as defined above.

For example, Z may be an one of the structures shown below:

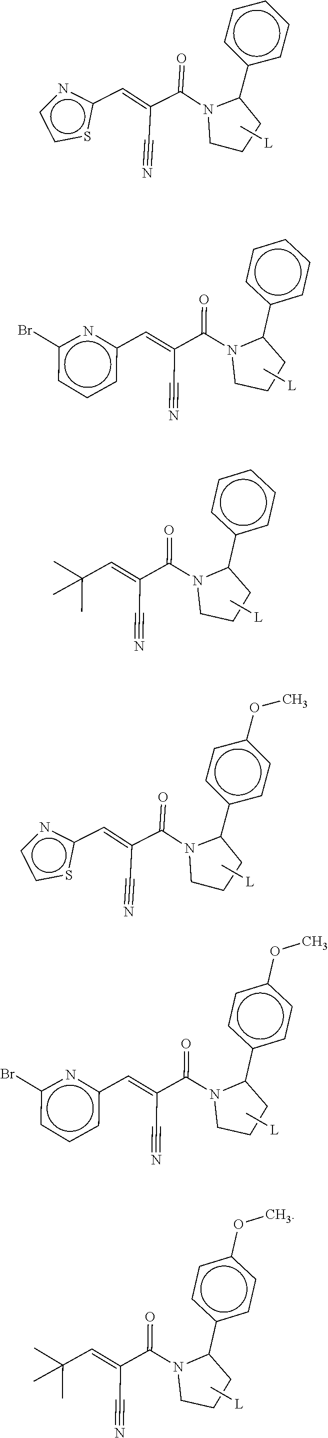

For example, Z may be one of the structure shown below:

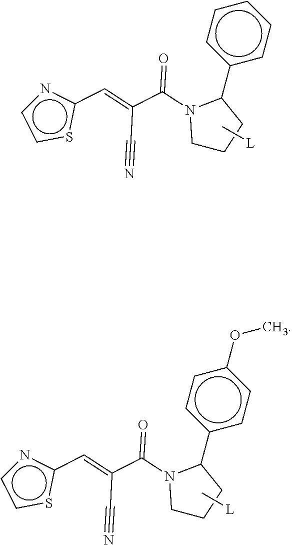

Alternatively it is noted, that whilst the various formulae (WZI) to (WZV), and (WI) to (WIV) (and sub-generic formulae thereof) described herein indicate that the linker is joined to the Z moiety via the heterocyclic core (either directly or indirectly via the R^{2A} group), the present disclosure also extends to examples wherein the linker is attached at any other position in the Z moiety (provided that it has the correct valency and/or is chemically suitable). For example, the linker may replace a hydrogen atom at any position in the Z moiety. Thus, in some examples, Z may be represented as shown in formula (WZV) or (WV):

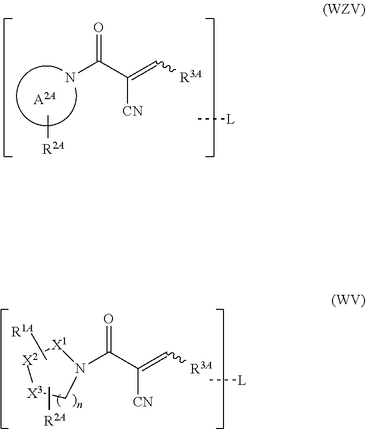

wherein ring A^{2A}, R^{1A}, R^{2A}, R^{3A}, X¹, X², X³, n and L are as defined for any of the embodiments of formula (W) or sub-generic formulae thereof (e.g. formula (WZI) or (WI) (or any of one or more of formulae (WZIa) to (WZIV) or (WIa) to (WIVa)).

The dotted line shown through the square brackets on formulae (WZV) and (WV) indicates that the linker may be joined via a covalent bond to any atom on the Z moiety provided that it has the correct valency, is chemically suitable and/or provided that the attachment of the linker at this alternative position does not disrupt the function of the Z moiety in promoting and/or facilitating proteasomal degradation.

As described above, in some embodiments, Z may comprise a structure according to formula (A):

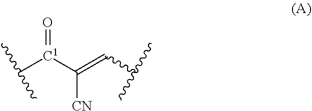

wherein the linker is attached to carbonyl carbon C¹;
in particular, in some embodiments, Z consists of, or consists essentially of, a structure according to formula (A1):

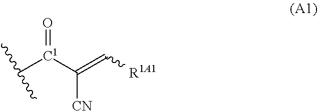

wherein R^{1A1} may be any suitable chemical group.

For example, R^{1A1} is selected from alkyl (e.g. C₁ to C₆ alkyl, e.g. t-Bu), cycloalkyl (e.g. cyclobutyl or cyclopentyl), heterocycloalkyl (e.g. morpholine, tetrahydrofuran or tetrahydropyran), substituted cycloalkyl, alkyl cycloalkyl (e.g. CH₂-cyclohecyl), substituted alkylcycloalkyl, alkyl heterocycloalkyl (e.g. CH₂-morpholine), substituted alkylheterocycloalkyl, aryl (e.g. benzene), substituted aryl, alkyl aryl (e.g. benzyl), substituted alkylaryl, heteroaryl (e.g. pyridyl), substituted heteroaryl, alkyl heteroaryl (e.g. CH₋ₚpyridyl), substituted alkylheteroaryl, alkyl amino (e.g. (CH₂)₂NMe₂), alkyl amide (e.g. (CH₂)₂N(Me)COMe), alkoxyalkyl ((CH₂)₂OMe), alkylcarbonyl (e.g. (CH₂)₂COMe), alkyl carboxylic acid ((CH₂)₃COOH), optionally wherein the alkyl (e.g. C₁ to C₆ alkyl) is substituted with one or more heteroatoms selected from halo, N, O and S; and
wherein the linker is attached to carbonyl carbon C¹.

In embodiments of the invention as defined by formula (A1) or any formulae herein defined, the term “substituted” in respect of substituted cycloalkyl, substituted alkylcycloalkyl, substituted heterocycloalkyl, substituted alkylheterocycloalkyl, substituted aryl, substituted alkylaryl, substituted heteroaryl and substituted alkylheteroaryl also encompasses monocyclic, bicyclic and tricyclic ring systems, wherein the further rings are joined by a covalent bond, at a fused ring junction, at a spiro ring junction, or via a bridged ring system, or any combination thereof.

In embodiments, Z consists, or consists essentially of, of a structure according to formula (A1), wherein R^{1A1} is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S, and/or is substituted with a carbocyclic or heterocyclic group.

In embodiments, R^{1A1} is selected from the group consisting of optionally substituted heteroaryl, C₁-C₆ alkyl, optionally substituted C₃-C₆ cycloalkyl, optionally substituted C₃-C₆ cycloheteroalkyl, C₁-C₆ alkyl substituted with a heterocyclic group, aryl, and substituted aryl.

In embodiments, R^{1A1} is selected from the group consisting of

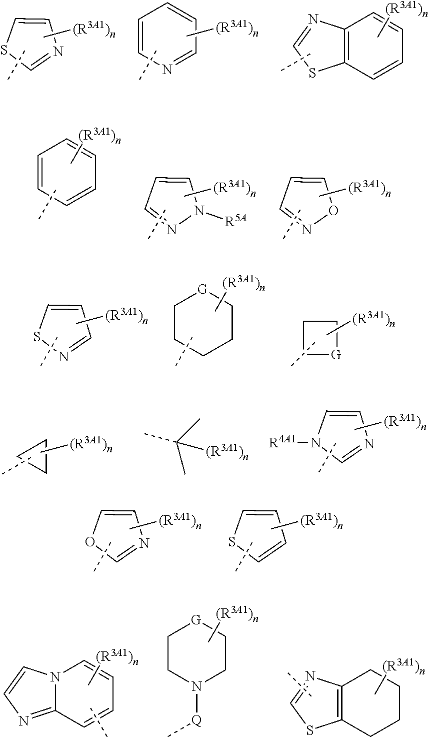

wherein the dotted line indicates the position at which each of the respective R¹ groups is joined to the structure shown in formula (I), or wherein when the dotted line is not appended to an atom, the dotted line indicates that each of the respective R^{1A1} group is joined to the structure via any position on the aromatic or heteroaromatic ring;
each R^{3A1} is independently selected from the group consisting of halo, CF₃, —CH₂F, —CHF₂, —OCF₃, —OCH₂F, —OCHF₂, C₁ to C₆ alkyl, —CN, —OH, —OMe, —SMe, —SOMe, —SO₂Me, —NH₂, —NHMe, —NMe₂, CO₂Me, —NO₂, CHO and COMe;
n is 0 to 3;
R^{4A1} is C₁ to C₆ alkyl;
G is CH₂, O or NH; and
Q is C₁ to C₆ alkylene.

In further embodiments, R^{1A1} is selected from the group consisting of:

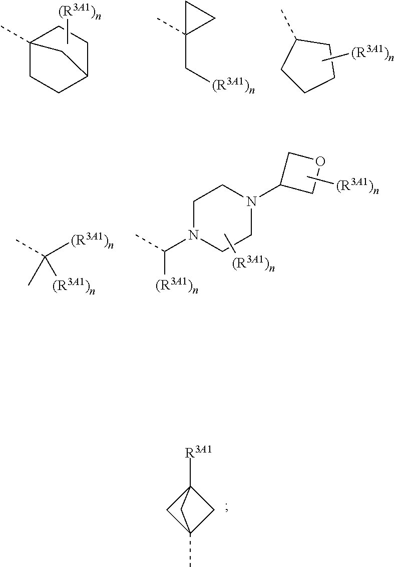

wherein R^{3A1} and n are as defined above.

In further embodiments, R^{1A1} is selected from the group consisting of:

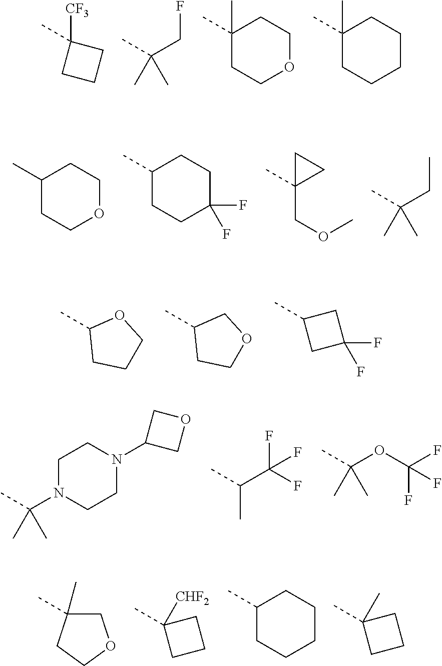

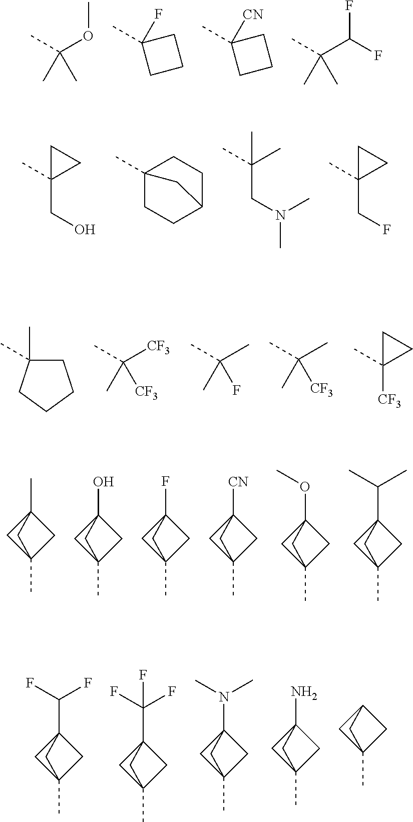

wherein the dotted line indicates the position at which each of the respective R^{1A1} groups is joined to the structure shown in the formulae described herein.

By way of another example, a suitable R^{1A1} group may be selected from the following:

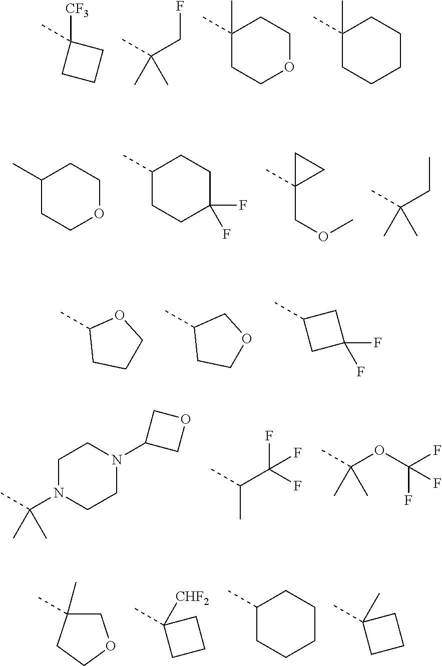

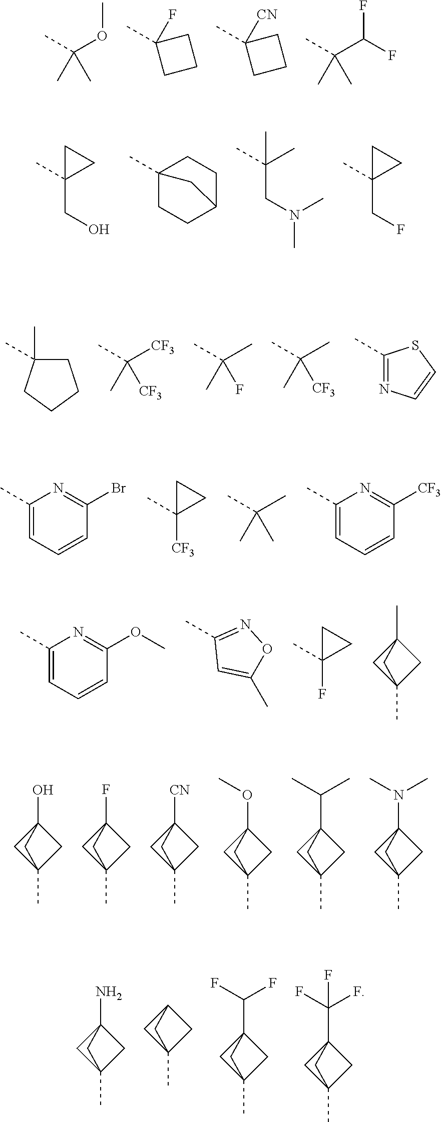

Again, in the structures shown above, the line intersected by a wavy line represents the covalent bond between the exemplary R^{1A1} groups shown above and the carbon atom of the parent structure of Z (as illustrated by the various formulae (A1) to (A3) (and sub-generic formulae thereof) described herein).

By way of another example, a suitable R^{1A1} group may be selected from the following:

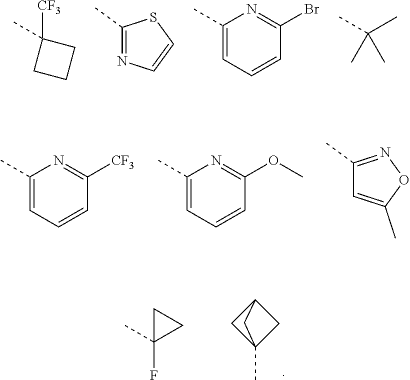

Again, in the structures shown above, the line intersected by a wavy line represents the covalent bond between the exemplary R^{1A1} groups shown above and the carbon atom of the parent structure of Z (as illustrated by the various formulae (A1) to (A3) (and sub-generic formulae thereof) described herein).

In the above embodiments, the atom directly attached to C¹ is suitably N.

In embodiments, the bifunctional molecule comprises a structure according to formula (A2):

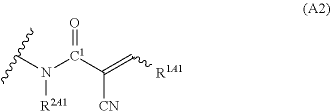

wherein
C¹ and R^{1A1} are defined as for formula (A1);
R^{2A1} is selected from H, C₁ to C₆ alkyl, alkylaryl, substituted alkylaryl, cycloalkyl, substituted cycloalkyl, heterocycloalkyl and substituted heterocycloalkyl, optionally wherein the C₁ to C₆ alkyl is substituted with one or more heteroatoms selected from halo, N, O and S and/or is substituted with a carbocyclic or heterocyclic group.

In embodiments, the bifunctional molecule comprises a structure according to formula (A2a):

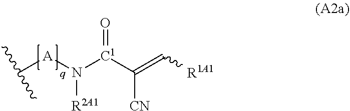

wherein
C¹ and R^{1A1} is as defined in formula (A1);
A is CR′R″;
R′ and R″ are each independently selected from H and C₁ to C₆ alkyl, optionally wherein the C₁ to C₆ alkyl is substituted with one or more heteroatoms selected from N, O or S, or wherein R′ and R″ together form a 3-, 4-, 5- or 6-membered carbocyclic or heterocyclic ring;
q is 1 to 3.

In embodiments of formula A2 and A2a, R^{2A1} is selected from H, C₁ to C₆ alkyl. In other embodiments, R^{2A1} is not H.

In alternative embodiments, the bifunctional molecule comprises a structure according to formula (A3):

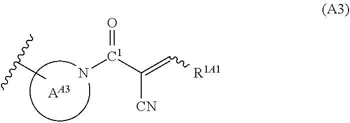

wherein:
C¹ and R^{1A1} is as defined in formula (A1);
ring A^{A3} is an optionally substituted monocyclic, bicyclic or tricyclic N-heterocycle optionally comprising one to four additional ring heteroatoms selected from N, O and S.

In embodiments, the bifunctional molecule comprises a structure according to formula (A3), wherein:
ring A^{A3} is an optionally substituted 4-membered to 9-membered (e.g. 5-membered to 6-membered) monocyclic N-heterocycloalkyl, optionally containing one or two additional ring heteroatoms selected from N, O and S; or
ring A^{A3} is an optionally substituted 6-membered to 12-membered (e.g. 7-membered to 8-membered) bridged N-heterocycloalkyl, optionally containing one or two additional ring heteroatoms selected from N, O and S; or
ring A^{A3} is an optionally substituted bicyclic N-heterocycloalkyl comprising a first ring and a second ring, the first ring being an optionally substituted 3-membered to 7-membered N-heterocycloalkyl, optionally containing one or two additional ring heteroatoms selected from N, O and S, and the second ring being an optionally substituted 3-membered to 7-membered cycloalkyl or N-heterocycloalkyl optionally containing one or two ring heteroatoms selected from N, O and S, wherein the first and second ring are joined at a spiro centre; or
ring A^{A3} is an optionally substituted fused bicyclic N-heterocycloalkyl comprising a first ring and a second ring, the first ring being an optionally substituted 4-membered to 9-membered N-heterocycloalkyl optionally containing one or two additional ring heteroatoms selected from N, O and S, and the second ring being an optionally substituted 4-membered to 9-membered cycloalkyl or heterocycloalkyl ring optionally containing one or two ring heteroatoms selected from N, O and S; or
ring A^{A3} is an optionally substituted fused bicyclic N-heterocycloalkyl comprising a first ring and a second ring, the first ring being an optionally substituted 4-membered to 9-membered N-heterocycloalkyl optionally containing one or two additional ring heteroatoms selected from N, O and S and the second ring being a 6-membered to 10-membered aryl, heteroaryl, substituted aryl or substituted heteroaryl.

In embodiments, the bifunctional molecule comprises a structure selected from the group consisting of:

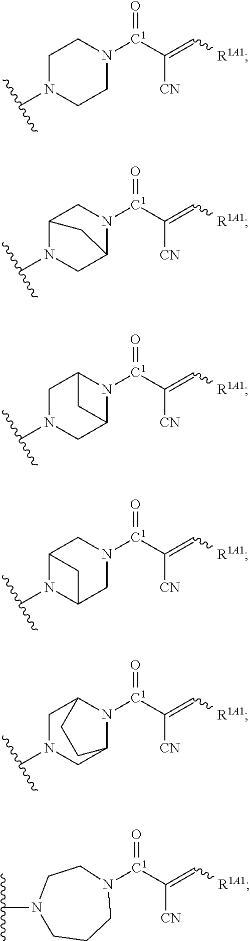

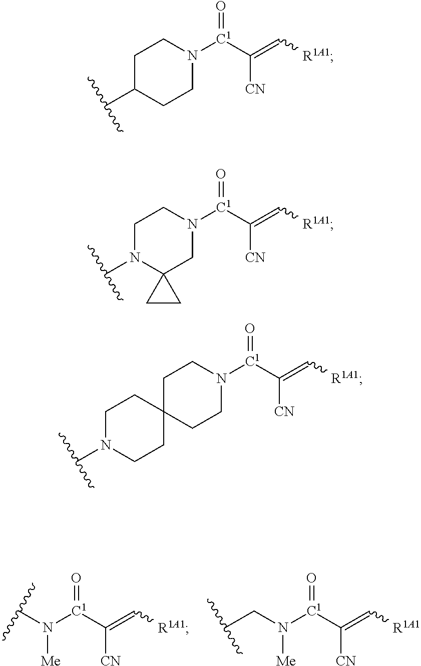

wherein C¹ and R^{1A1} is as defined in formula (A1).

In embodiments, the bifunctional molecule comprises a structure selected from the group consisting of:

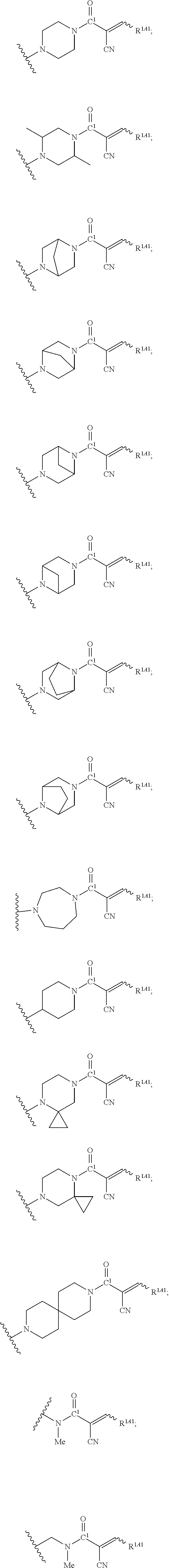

wherein C¹ and R^{1A1} is as defined in formula (A1).

In embodiments, the bifunctional molecule comprises a structure selected from the group consisting of:

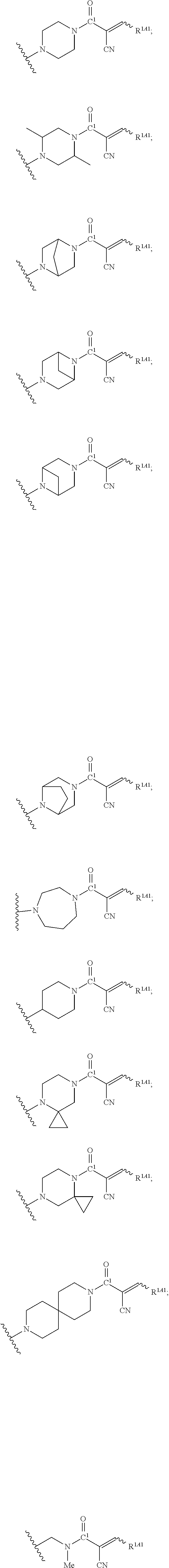

wherein C¹ and R^{1A1} is as defined in formula (A1).

In embodiments, the bifunctional molecule comprises a structure selected from the group consisting of:

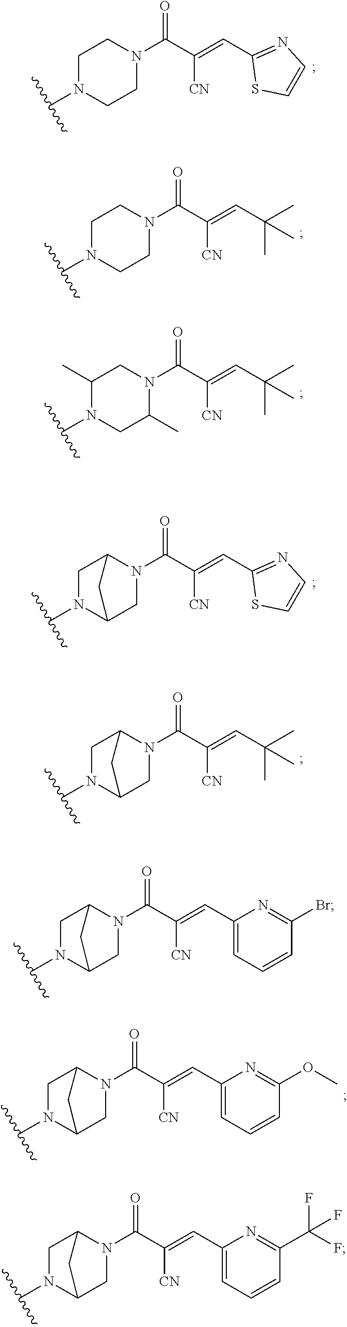

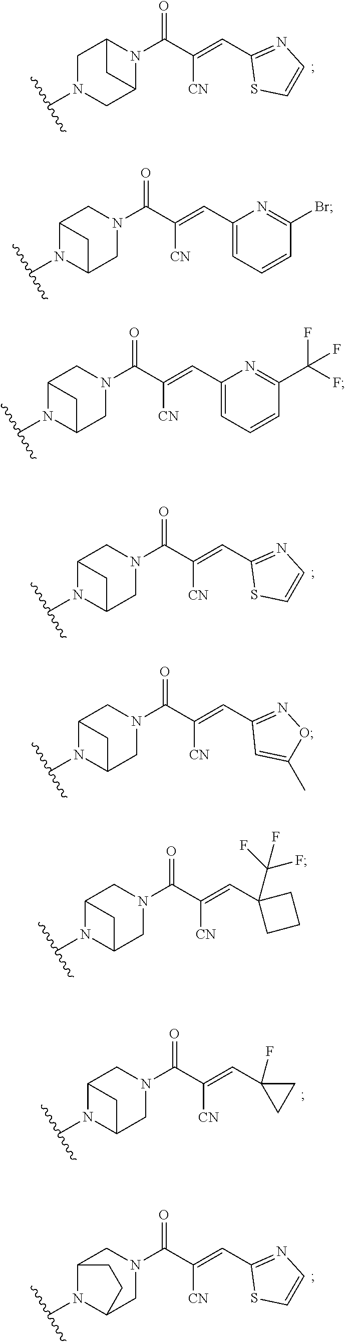

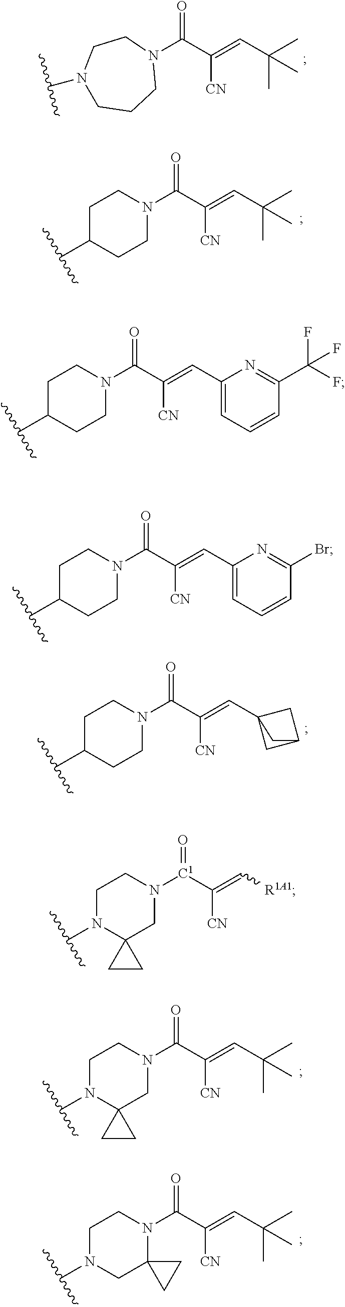

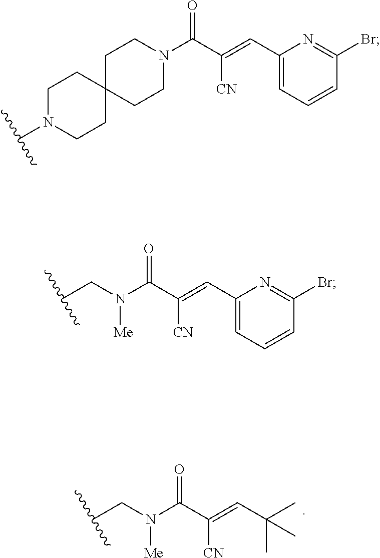 [SMILES: *N1CCC2(CC1)CCN(C(=O)/C(C#N)=C/c1cccc(Br)n1)CC2 ; *CN(C)C(=O)/C(C#N)=C/c1cccc(Br)n1 ; *CN(C)C(=O)/C(C#N)=C/C(C)(C)C]

### Linker (L)

As described herein, the TBL is linked or coupled to moiety Z via a linker L. The linker may be a chemical linker (e.g. a chemical linker moiety) and, for example, may be a covalent linker, by which is meant that the linker is coupled to Z and/or TBL by a covalent bond.

The linker acts to tether the target protein binding ligand and Z moieties to one another whilst also allowing both of these portions to bind to their respect targets and/or perform their intended function. In particular, the linker may act to tether the target protein binding ligand to Z whilst also mitigating the possibility of the Z moiety disrupting, interfering with and/or inhibiting the binding of the target protein binding ligand to the target protein. Additionally or alternatively, the linker may act to tether Z to the target protein binding ligand whilst also mitigating the possibility of the target protein binding ligand disrupting, interfering with and/or inhibiting the cellular interactions of Z (e.g. its function in modulating, facilitating and/or promoting the proteasomal degradation of the target protein).

In other words, the linker may function to facilitate targeted protein degradation by allowing each end of the bifunctional molecule to be available for binding (or another type of cellular interaction) with various components of the cellular environment. For example, the linker may be configured to allow the target protein binding ligand to bind to the target protein without interference, disruption and/or inhibition from the Z moiety of the bifunctional molecule. Additionally or alternatively, the linker may be configured to allow the Z moiety to interact with the various components in the cellular environment to modulate, facilitate and/or promote the proteasomal degradation of the target protein without interference, disruption and/or inhibition from the target protein binding ligand of the bifunctional molecule.

In many cases, a broad range of linkers will be tolerated. The selection of linker may depend upon the protein being targeted for degradation (the target protein) and/or the particular target protein binding ligand that binds to BRD9.

The linker may be selected to provide a particular length and/or flexibility, e.g. such that the target protein binding ligand and the Z moiety are held within a particular distance and/or geometry. As will be appreciated by one of skill in the art, the length and/or flexibility of the linker may be varied dependent upon the structure and/or nature of the target protein binding ligand.

In some examples, the TBL is connected directly to moiety Z by a covalent bond i.e, the linker is a covalent bond. Such a direct connection is also encompassed within the term “linker” within the context of the present disclosure (and unless otherwise stated).

By way of example only, the linker may comprise any number of atoms between 1 and 200, between 1 and 100, between 1 and 50, between 1 and 30 or between 1 and 10. In some cases the linker may comprise any number of atoms in a single linear chain of between 1 and 200, between 1 and 100, between 1 and 50, between 1 and 30 or between 1 and 10. In some examples of the disclosure, the linker may comprise any number of atoms in a single linear chain between 1 and 25, such as 3 and 25, or between 1 and 20, such as 3 and 20, or between 1 and 18, such as 3 and 18.

The degree of flexibility of the linker may depend upon the number of rotatable bonds present in the linker. A rotatable bond is defined as a single non-ring bond, bound to a nonterminal heavy atom (e.g. non-hydrogen atom). As described herein, an amide (C—N) bond is not considered rotatable because of the high rotational energy barrier. In some cases, the linkers may comprise one or more moieties selected from rings, double bonds and amides to reduce the flexibility of the linker. In other cases, the linker may comprise a greater number and/or proportion of single bonds (e.g. may predominantly comprise single non-ring bonds) to increase the flexibility of the linker. It may also be appreciated that the length of the linker may affect the degree of flexibility. For example, a shorter linker comprising fewer bonds may also reduce the flexibility of a linker.

In some examples, the number of rotatable bonds present in the linker may be any number between 1 and 20, between 1 and 15, between 1 and 10, or between 1 and 8. In some examples, the number of rotatable bonds present in the linker may be any number between 2 and 9, between 2 and 8, or between 3 and 6.

In some examples, the linker may comprise any number of atoms in a single linear chain between 10 and 20; and/or the number of rotatable bonds present in the linker may be any number between 1 and 8.

The structure of the linker (L) may be represented as follows:

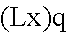

wherein each Lₓ represents a subunit of L; and
q is an integer greater than or equal to 1.

For example, q may be any integer between 1 and 30, between 1 and 20 or between 1 and 5.

By way of example, in the case where q is 1, the linker comprises only one Lₓ subunit and may be represented as L₁. In the case where q is 2, the linker comprises two Lₓ subunits that are covalently linked to one another and which may be represented as L₁-L₂. In another example, where q is 3, the linker comprises three L, subunits that are covalently linked to one another and may be represented as L₁-L₂-L₃. For even higher integer values of q, L may comprise the following subunits L₁, L₂, L₃, L₄ . . . up to L_{q}.

Each of Lₓ may be independently selected from CR^{L1}R^{L2}, O, C═O, S, S═O, SO₂, NR^{L3}, SONR^{L4}, SONR^{L5}C═O, CONR^{L6}, NR^{L7}CO, C(R^{L8})═C(R^{L9}), C≡C, aryl, substituted aryl, heteroaryl, substituted heteroaryl, carbocyclyl, substituted carbocyclyl, heterocyclyl and substituted heterocyclyl groups.

Each of R^{L1}, R^{L2}, R^{L3}, R^{L4}, R^{L5}, R^{L6}, R^{L7}, R^{L8}, and R^{L9} may be independently selected from H, halo, C₁ to C₆ alkyl, C₁ to C₆, haloalkyl, —OH, —O(C₁ to C₆ alkyl), —NH₂, —NH(C₁ to C₆ alkyl), —NO₂, —CN, —CONH₂, —CONH(C₁ to C₆ alkyl), —CON(C₁ to C₆ alkyl)₂, —S(O)OC₁ to C₆ alkyl, —C(O)OC₁ to C₆ alkyl, and —CO(C₁ to C₆ alkyl). In some examples, each of R^{L1}, R^{L2}, R^{L3}, R^{L4}, R^{L5}, R^{L6}, R^{L7}, R^{L8} and R^{L9} may be independently selected from H and C₁ to C₆ alkyl.

The terminal Lₓ subunits may link or couple the linker moiety to the TBL and Z moieties of the bifunctional molecule. For example, if the terminal L, subunits are designated as L₁ and L_{q}, L₁ may link the linker to the TBL moiety and L_{q} may link the linker to the Z moiety. In those cases where q is 1, the one Lₓ subunit (e.g. L₁) provides the link between the TBL and Z moieties of the bifunctional molecule.

The TBL and Z moieties may be covalently linked to L through any group which is appropriate and stable to the chemistry of the linker. By way of example only, the linker may be covalently bonded to the TBL moiety via a carbon-carbon bond, keto, amino, amide, ester or ether linkage. Similarly, the linker may be covalently bonded to the Z moiety via a carbon-carbon bond, carbon-nitrogen bond, keto, amino, amide, ester or ether linkage.

In some cases, each terminal Lₓ subunit (e.g. L₁ and L_{q}) is independently selected from O, C═O, CR^{L1}R^{L2}, NR^{L3}, CONR^{L6}, NR^{L7}CO, aryl, substituted aryl, heteroaryl, substituted heteroaryl, carbocyclyl, substituted carbocyclyl, heterocyclyl and substituted heterocyclyl groups.

In some examples, at least one of Lₓ comprises a ring structure and is, for example, selected from a heterocyclyl, heteroaryl, carbocyclyl or aryl group.

In alternative examples, the linker may be or comprise an alkyl linker comprising, a repeating subunit of —CH₂—; where the number of repeats is from 1 to 50, for example, 1-50, 1-40, 1-30, 1-20, 1-19, 1-18, 1-17, 1-16, 1-15, 1-14, 1-13, 1-12, 1-11, 1-10, 1-9. 1-8, 1-7, 1-6, 1-5, 1-4, 1-3 and 1-2.

In other examples, the linker may be or comprise a polyalkylene glycol. By way of example only, the linker may be or comprise a polyethylene glycol (PEG) comprising repeating subunits of ethylene glycol (C₂H₄O), for example, having from about 1-50 ethylene glycol subunits, for example where the number of repeats is from 1 to 100, for example, 1-50, 1-40, 1-30, 1-20, 1-19 1-18, 1-17, 1-16, 1-15, 1-14, 1-13, 1-12 or 1-5 repeats.

In some of the examples described herein, the structure of the linker (L) may be, or comprise, a structure represented as shown in formula (L1a):

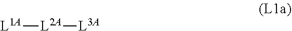

wherein L^{1A} is absent or is selected from C₁-C₆ alkylene (e.g. ethylene), C₁-C₆ alkoxy (e.g. —O(CH₂)—, —O(CH₂)₂—, —O(CH₂)₅—, —CH₂OCH₂₋) and C₁-C₆ alkylamino (e.g. —NR^{L2A}(CH₂)—, —R^{L2A}(CH₂)₂—, —R^{L2A}(CH₂)₅—, —CH₂R^{L2A}CH₂—);
L^{2A} is —NR^{L2A}C═O— or —C═ONR^{L2A}—; and
L^{3A} is selected from C₁-C₃ alkylene (e.g. ethylene), C₁-C₆ alkoxy (e.g. —(CH₂)O—, —(CH₂)₂O—, —(CH₂)₅O—, —CH₂OCH₂—) and C₁-C₆ alkylamino (e.g. —(CH₂)NR^{L2A}—, —(CH₂)₂NR^{L2A}—, —(CH₂)₅NR^{L2A}—, —CH₂NR^{L2A}CH₂—);
   wherein R^{L2A} is H or C₁-C₆ alkyl (e.g. C₁-C₃ alkyl).

In further examples, the structure of the linker (L) may be, or comprise, a structure represented as shown in formula (L1b):

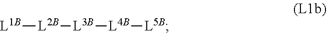

wherein L^{1B} is absent or is selected from C₁-C₃ alkylene (e.g. ethylene), C₁-C₆ alkoxy (e.g. —O(CH₂)—, —O(CH₂)₂—, —O(CH₂)₅—, —CH₂OCH₂—) and C₁-C₆ alkylamino (e.g. —NR^{L2A}(CH₂)—, —NR^{L2A}(CH₂)₂—, —R^{L2A}(CH₂)₅—, —CH₂R^{L2A}CH₂—);
L^{2B} is —NR^{L2A}C═O— or —C═ONR^{L2A}—;
L^{3B} is selected from C₁-C₁₅ alkylene, —[(CH₂)₂O]₁₋₆(CH₂)₂—;
L^{4B} is —NR^{L2A}C═O— or —C═ONR^{L2A}— wherein R^{L2A} is H or C₁-C₆ alkyl (e.g. C₁-C₃ alkyl);
L^{5B} is selected from C₁-C₃ alkylene (e.g. ethylene), C₁-C₆ alkoxy (e.g. —(CH₂)O—, —(CH₂)₂O—, —(CH₂)₅O—, —CH₂OCH₂—) and C₁-C₆ alkylamino (e.g. —(CH₂)NR^{L2A}—, —NR^{L2A}(CH₂)₂—, —(CH₂)₅NR^{L2A}, —CH₂NR^{L2A}CH₂—);
   wherein R^{L2A} is H or C₁-C₆ alkyl (e.g. C₁-C₃ alkyl).

In some of the examples described herein, the structure of the linker (L) may be, or comprise, a structure represented as shown in formula (L1c):

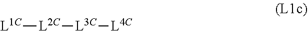

wherein L^{1C} is an optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from N, O and S;
L^{2C} is absent or is selected from C₁-C₃ alkylene (e.g. ethylene), C₁-C₆ alkoxy (e.g. —(CH₂)O—, —(CH₂)₂O—, —(CH₂)₅O—, —CH₂OCH₂—) and C₁-C₆ alkylamino (e.g. —(CH₂)NR^{L2A}—, —(CH₂)₂NR^{L2A}—, —(CH₂)₅NR^{L2A}—, —CH₂NR^{L2A}CH₂—);
L^{3C} is —R^{L2B}C═O— or —(C═O)R^{L2B}—; and
L^{4C} is selected from C₁-C₃ alkylene (e.g. ethylene), C₁-C₆ alkoxy (e.g. —(CH₂)O—, —(CH₂)₂O—, —(CH₂)₅O—, —CH₂OCH₂—) and C₁-C₆ alkylamino (e.g. —(CH₂)NR^{L2A}—, —(CH₂)₂NR^{L2A}—, —(CH₂)₅NR^{L2A}—, —CH₂NR^{L2A}CH₂—);
   wherein:
   R^{L2A} is H or C₁-C₆ alkyl (e.g. C₁-C₃ alkyl); and
   R^{L2B} is NR^{L2A}; or an N-linked optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from N, O and S.

In examples of Linker (L) represented by the Formula L1c, L^{1C} and L^{2C} may be both absent. In such examples, R^{L2B} in L^{3C} is an N-linked optionally substituted 4- to 7-membered monocyclic N-heterocycloalkyl, optionally containing one or two additional ring heteroatoms selected from N, O and S, and L^{3C} is the terminal subunit of the linker attached, suitably covalently attached, to the TBL via R^{L2B}.

In some of the examples described herein, the structure of the linker (L) may be, or comprise, a structure represented as shown in formula (L1d):

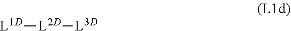

wherein L^{1D} is absent or is selected from C₁-C₃ alkylene, CO, C₁-C₃ alkylene(N(C₁-C₃ alkyl);
L^{2D} is NR^{L2A} or an optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from N, O and S; wherein R^{L2A} is H or C₁-C₆ alkyl (e.g. C₁-C₃ alkyl); and
L^{3D} is absent or is selected from C₁-C₃ alkylene, —O—, —N(C₁-C₃ alkyl)-, and CO.

In further examples, the structure of the linker (L) may be, or comprise, a structure represented as shown in formula (L1e):

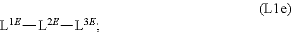

wherein L^{1E} is C₁-C₃ alkylene (e.g. methylene) or CO;
L^{2E} is an optionally substituted 4- to 7-membered monocyclic N-heterocycloalkyl, an optionally substituted 7- to 12-membered bicyclic N-heterocycloalkyl, each optionally containing one or two additional ring heteroatoms selected from N, O and S; and
L^{3E} is selected from C₁-C₃ alkylene (e.g. methylene).

In some examples, L^{1A}, L^{1B}, L^{1C}, L^{1D}, or L^{1E} is the terminal subunit of the linker structure attached (i.e. covalently bonded) to the W moiety and L^{3A}, L^{5B}, L^{4C}, L^{3D}, L^{3E}, is the terminal subunit of the linker structure attached (i.e. covalently bonded) to the TBL portion.

Where any of L^{1A}, L^{1B} or L^{1D} are absent, L^{2A}, L^{2B} or L^{2D} is directly attached (i.e. covalently bonded) to the W moiety. Where L^{3D} is absent, L^{2D} is directly attached (i.e. covalently bonded) to the TBL portion.

As stated above, a number of linker portions, such as L^{1C}, L^{2D}, L^{2E} examples of R^{L2B} and, may be bicyclic or tricyclic, and unless otherwise stated, these moieties may comprise rings that are joined by a bond, rings that are fused, a bridged ring and/or rings that are joined at a spiro centre.

When any one of L^{1C}, L^{2D}, L^{2E} examples of R^{L2B} is bicyclic, it may be a bridged bicyclic ring (i.e. it may comprise two rings that share three or more atoms) or it may be a spirocyclic bicyclic ring (i.e. it may comprise two rings that share one atom, e.g. the two rings may be joined at a spiro centre).

When any one of L^{1C}, L^{2D}, L^{2E} examples of R^{L2B} is a bridged bicyclic ring, it may be an optionally substituted 7- to 12-membered bridged bicyclic N-heterocycloalkyl optionally containing one or two additional ring heteroatoms selected from N, O and S. In some examples, L^{1C}, L^{2D}, L^{2E}, and examples of R^{L2B} may be a 7- or 8-membered bridged bicyclic N-heterocycloalkyl optionally containing one or two additional ring heteroatoms selected from N, O and S. In some examples, L^{1C}, L^{2D}, L^{2E}, and examples of R^{L2B} may be a 7- or 8-membered bridged bicyclic N-heterocycloalkyl optionally containing one additional ring atom selected from N.

When any one of L^{1C}, L^{2D}, L^{2E}, and examples of R^{L2B} is a spirocyclic bicyclic ring, it may be an optionally substituted 7- to 12-membered spirocyclic bicyclic N-heterocycloalkyl optionally containing one or two additional ring heteroatoms selected from N, O and S. In some examples, L^{1C}, L^{2D}, L^{2E}, and examples of R^{L2B} may be a 7- to 12-membered spirocyclic bicyclic N-heterocycloalkyl optionally containing one or two additional ring heteroatoms selected from N, O and S. In some cases, L^{1C}, L^{2D}, L^{2E}, and examples of R^{L2B} may be bicyclic and comprises a first 5- to 7-membered ring and a second 3- to 7-membered ring. For example, L^{1C}, L^{2D}, L^{2E}, and examples of R^{L2B} may be a spirocyclic bicyclic N-heterocycloalkyl comprising a first 5- or 6-membered ring and a second 3- to 6-membered ring, and optionally containing one or two additional ring heteroatoms selected from N, O and S. In some examples, L^{1C}, L^{2D}, L^{2E}, and examples of R^{L2B} may be a spirocyclic bicyclic N-heterocycloalkyl comprising a first 5- or 6-membered ring and a second 3- to 6-membered ring, and optionally containing one additional ring heteroatoms selected from N.

In some examples, the structure of L^{1C}, L^{2D}, L^{2E}, and examples of R^{L2B} may be any one selected from:

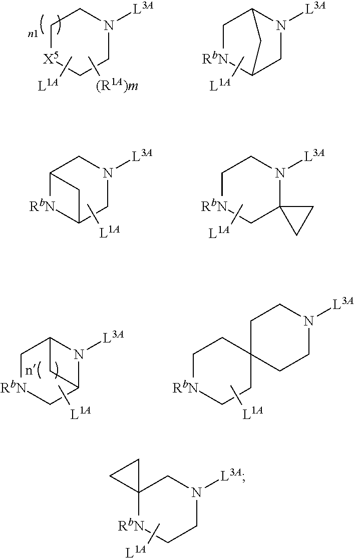

Wherein L^{1A} and L^{3A} are as defined above;
X⁵ is C(R^{b})₂, NR^{b} or O;
R^{b} is H or optionally substituted C₁₋₃alkyl;
n1 is 0, 1, 2 or 3;
n′ is 1 or 2;
m is 0, 1 or 2

The dotted line on the structures above indicates that the linker may be joined to the structure shown at any position indicated (provided that it has the correct valency and/or is chemically suitable).

In some examples L^{1C}, L^{2D}, L^{2E}, and examples of R^{L2B} is any one selected from:

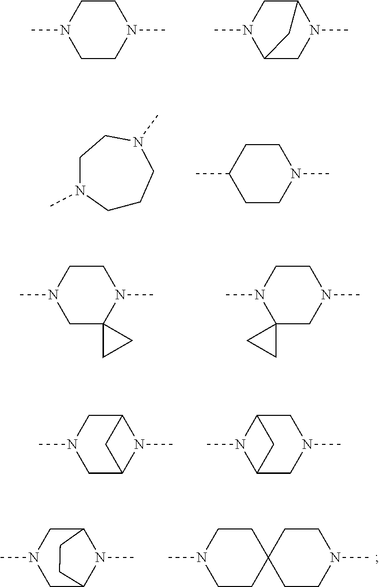

The dotted line on the structures above indicates that the linker may be joined to the structure shown at any position indicated (provided that it has the correct valency and/or is chemically suitable).

As stated above, L^{1D} is absent or is selected from C₁-C₃ alkylene, —O—, —N(C₁-C₃ alkyl)-, and CO.

In some examples, L^{3D} is selected from C₁-C₃ alkylene (e.g. methylene).

In some of the examples described herein, the linker (L) may be, or comprise, a structure represented as shown in formula (L1f):

L^{1F} (L1f)

wherein L^{1F} is selected from C₁-C₃ alkylene, CO, and C₁-C₃ alkylene(NR^{L1C}); wherein R^{L1C} is H or C₁-C₃ alkyl.

In some examples, L^{1F} is selected from C₁-C₃ alkylene (such as methylene).

In any of the examples described herein, the linker is or comprises one or more of:

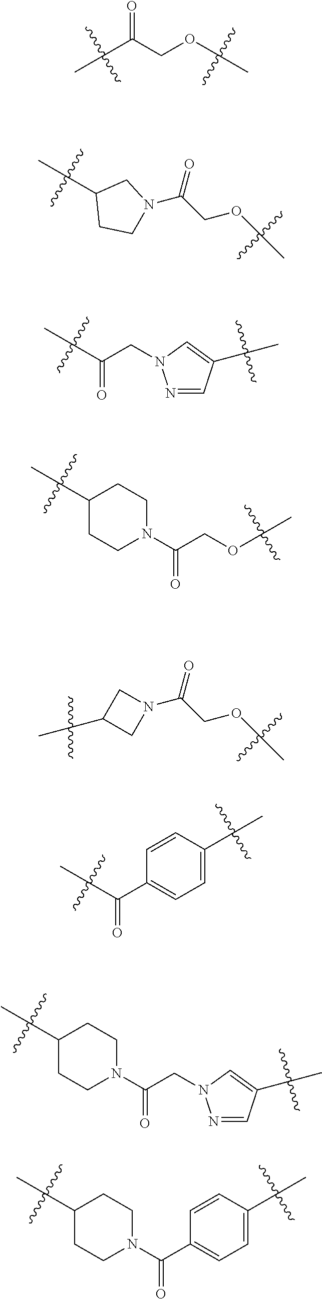

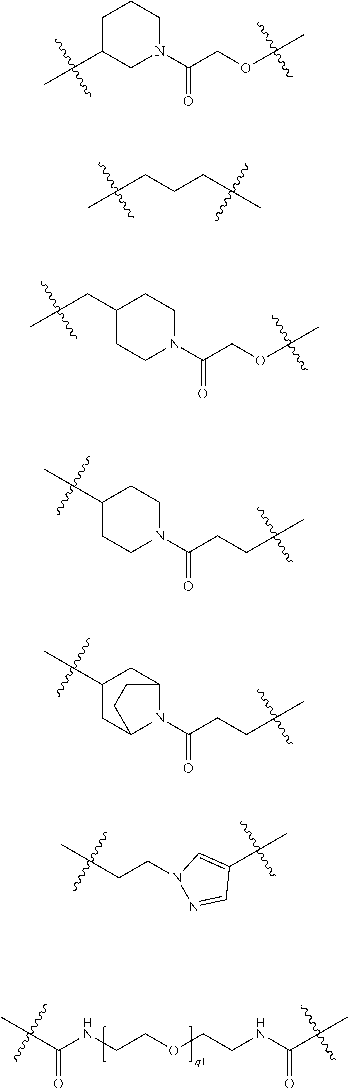

wherein q1 is any integer between 1 and 20, or between 1 and 10 (e.g. between 1 and 5).

Alternatively, in any of the examples described herein, the linker is or comprises one or more of:

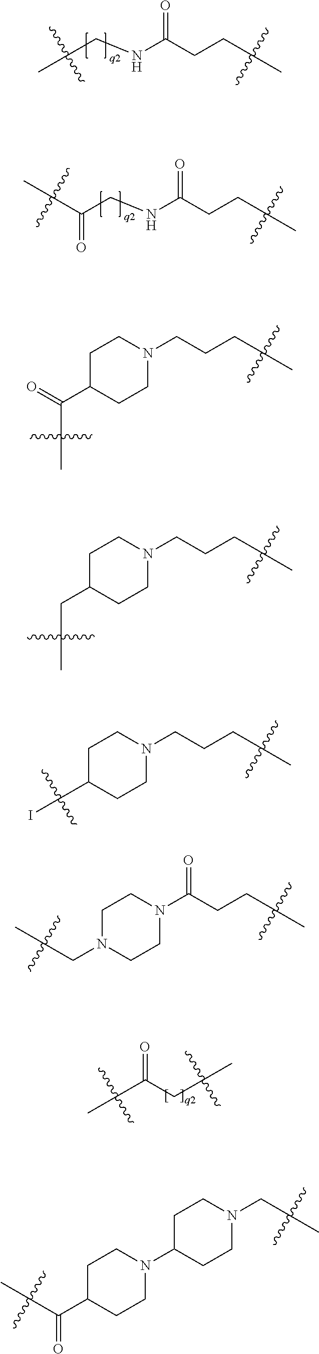

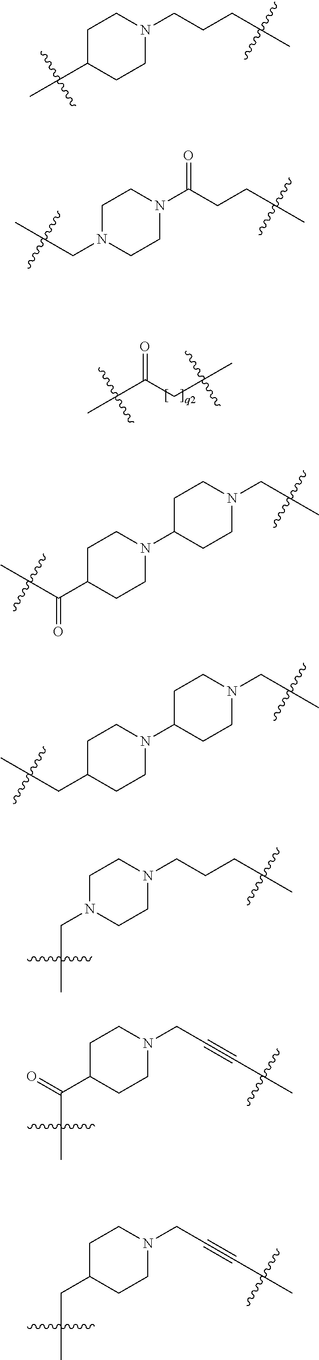

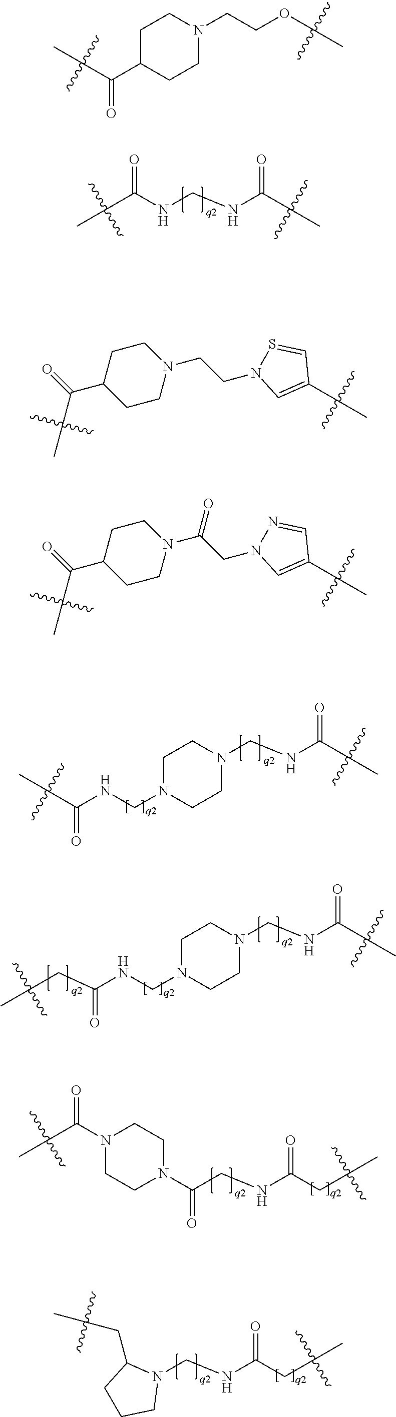

wherein q2 is any integer between 1 and 20, or between 1 and 10 (e.g. 3, 4, 6 or 10).

As a further alternative, in any of the examples described herein, the linker is or comprises one or more of:

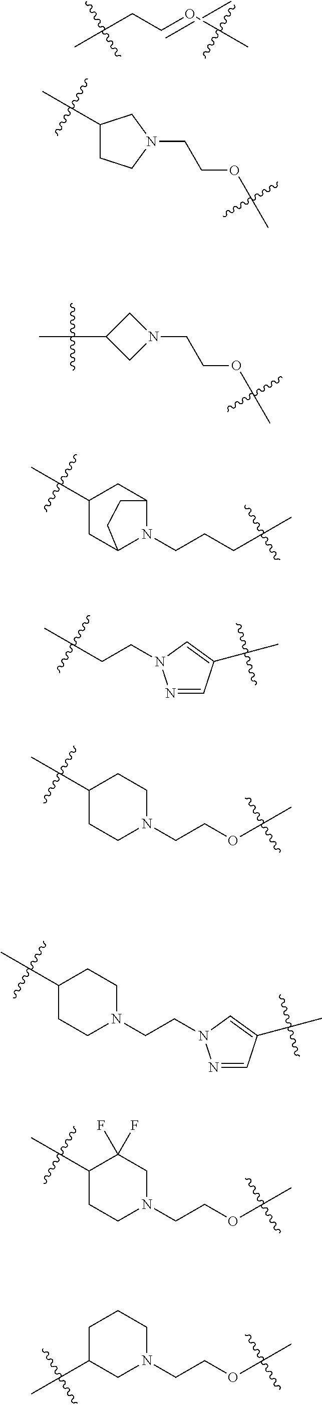

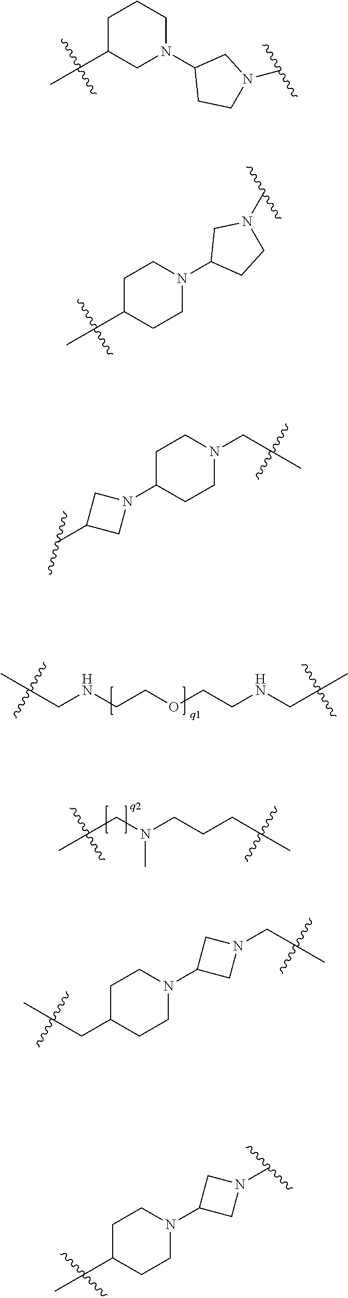

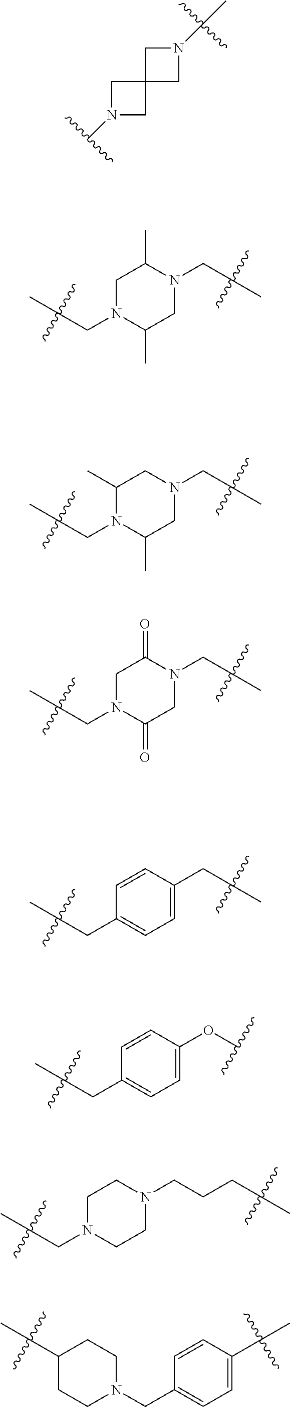

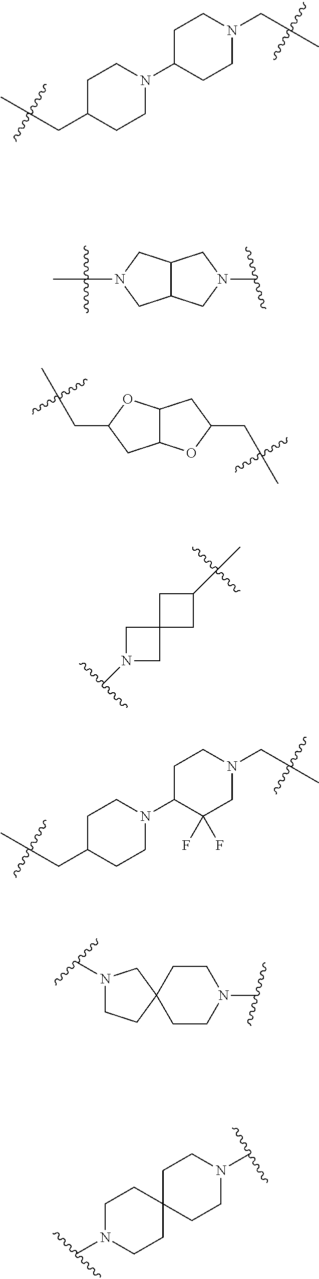

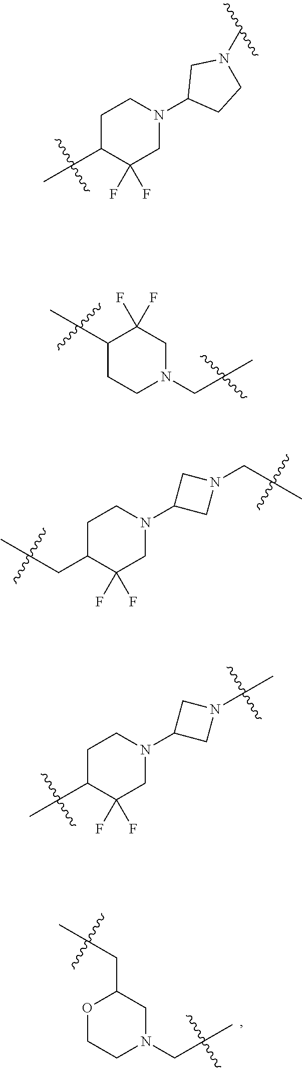

wherein q1 is any integer between 1 and 20, or between 1 and 10 (e.g. between 1 and 5) and q2 is any integer between 1 and 20, or between 1 and 10 (e.g. 3, 4, 5, 6 or 10).

In particular examples, the linker is or comprises one or more of the following structures:

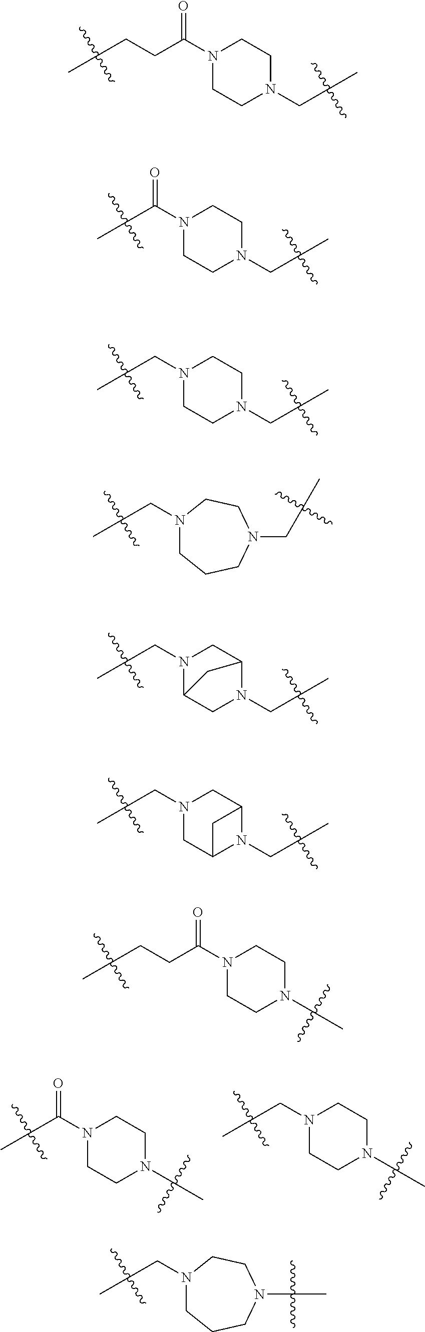

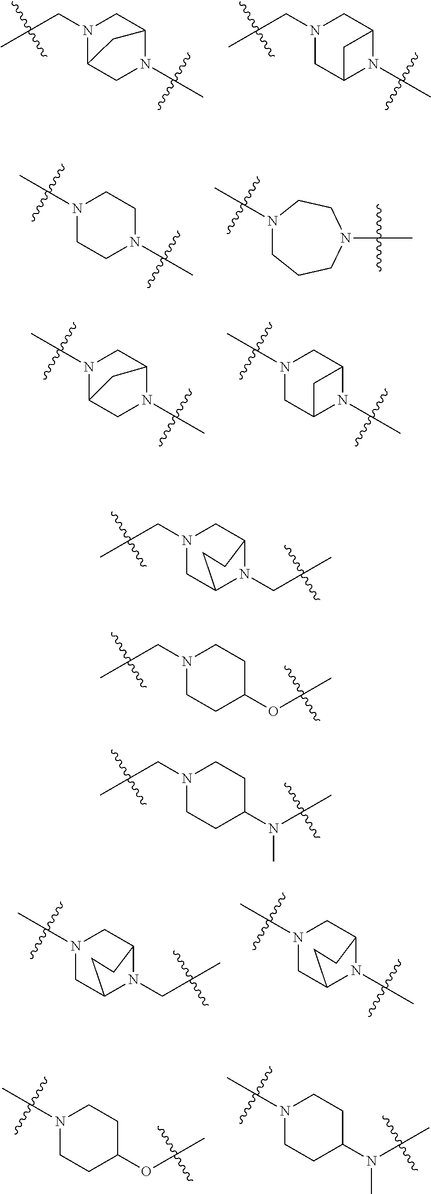

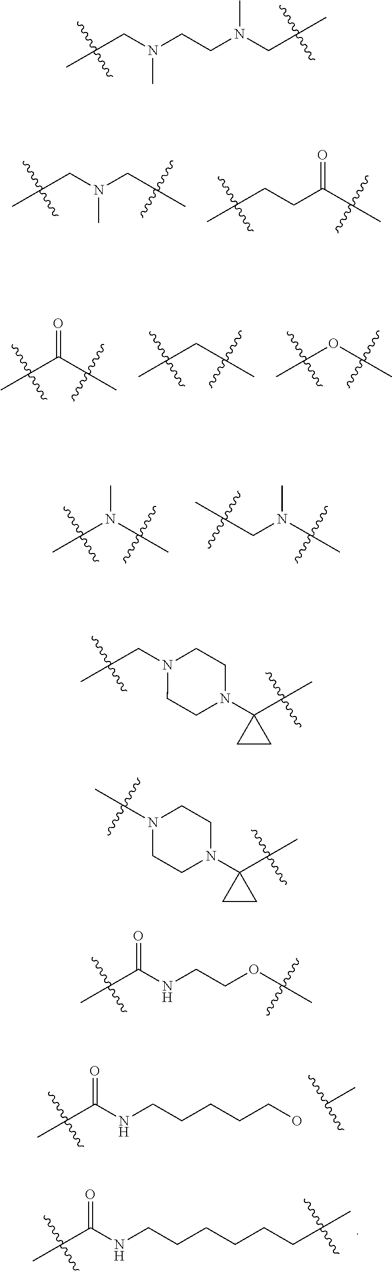

In yet further alternatives, in any of the examples described herein, the linker is or comprises one or more of:

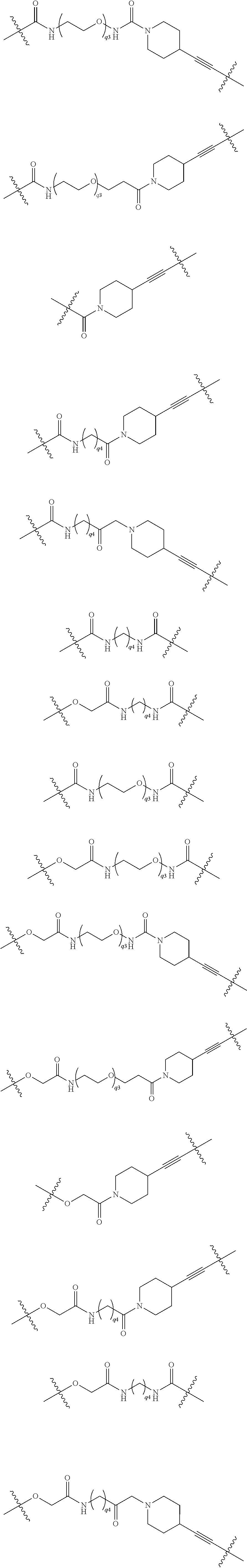

wherein q3 is 1 to 8, such as 1 to 5, and q4 is 1 to 12, such as 1 to 10.

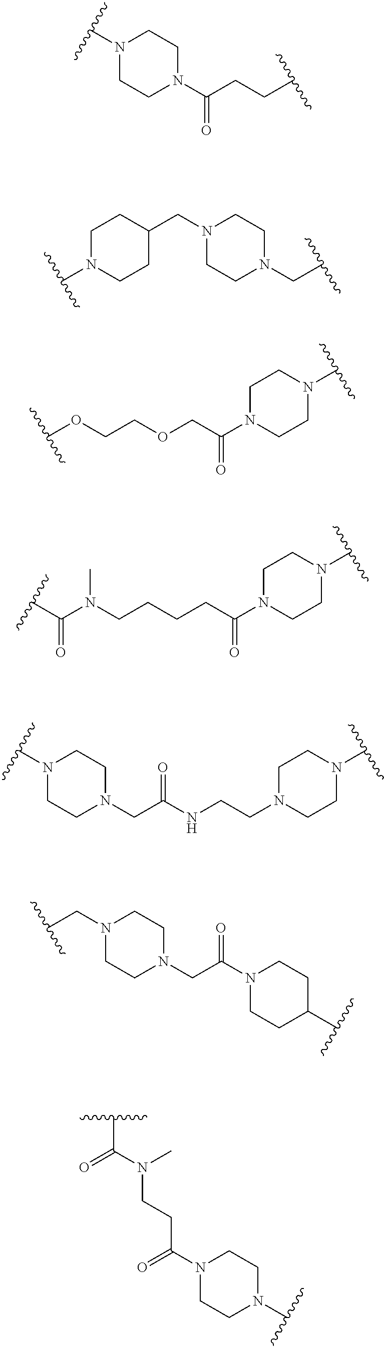

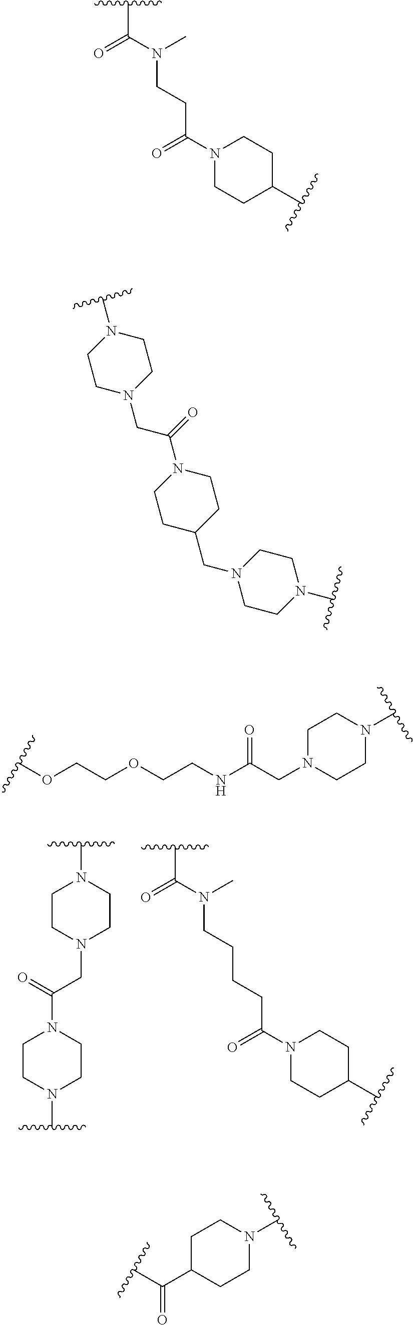

In particular examples, the linker is or comprises one or more of the following structures:

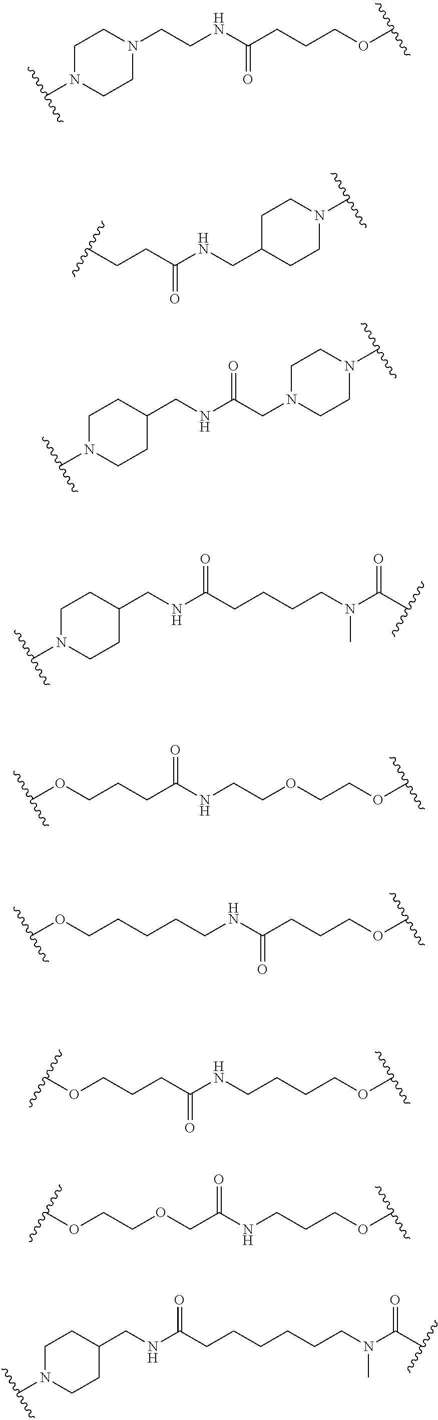

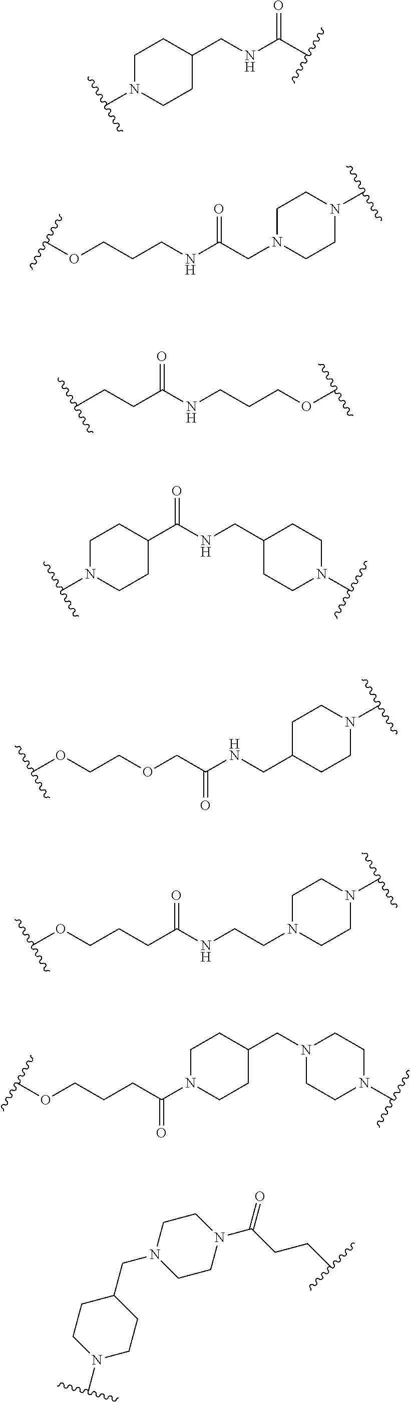

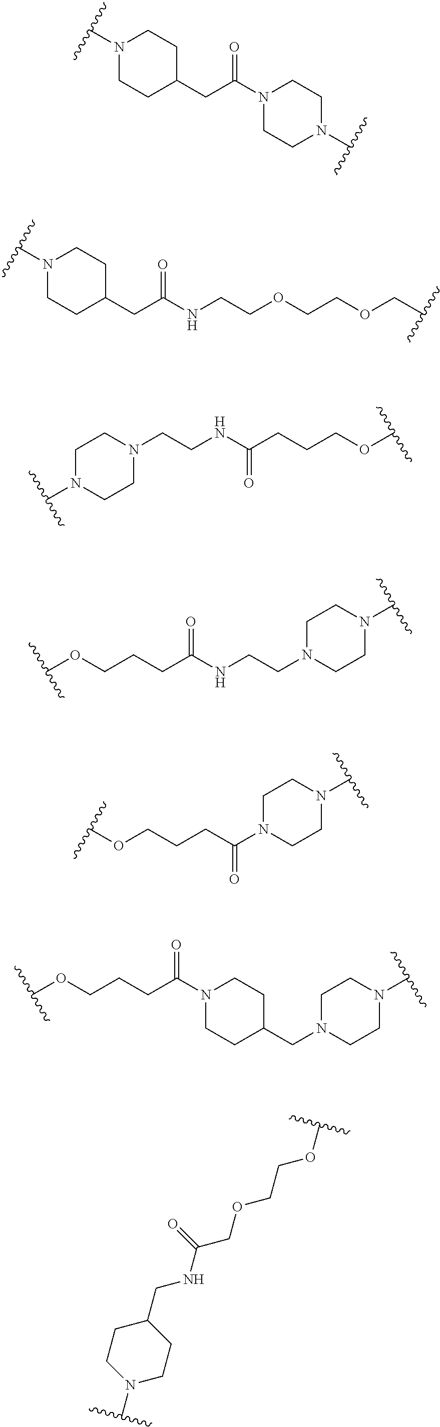

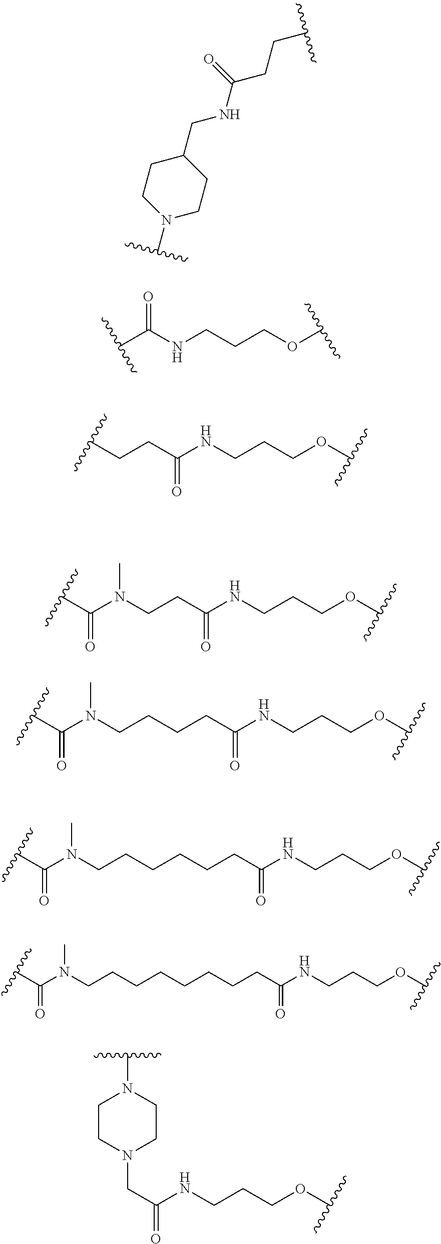

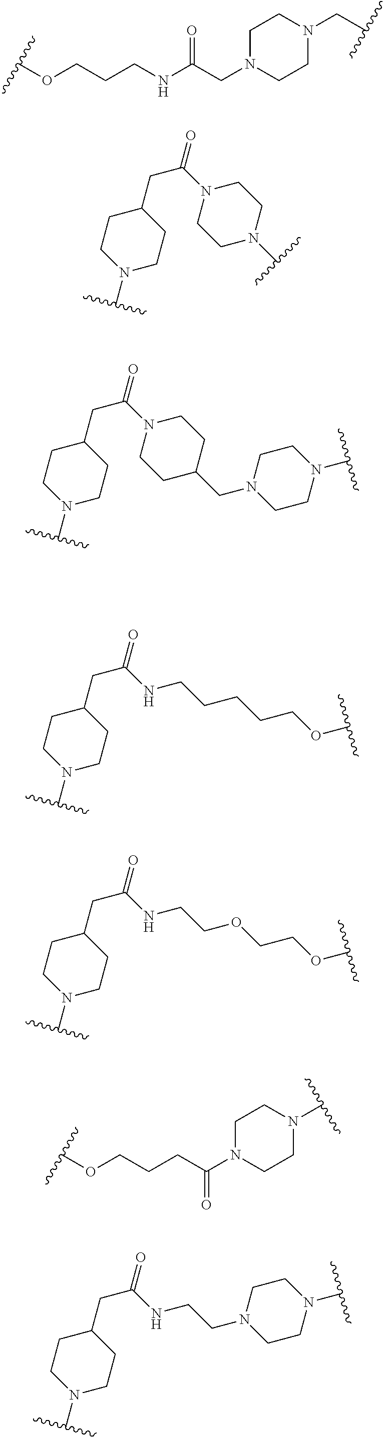

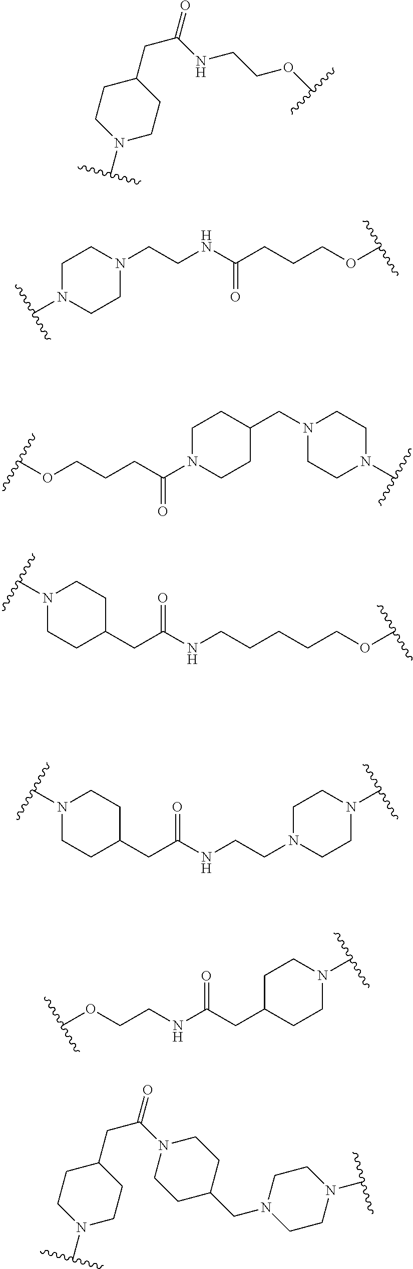

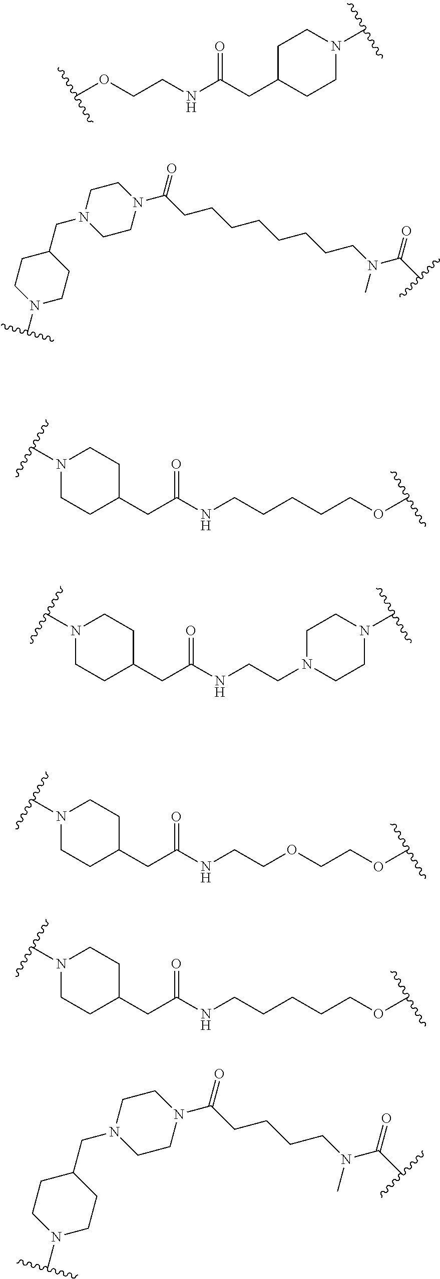

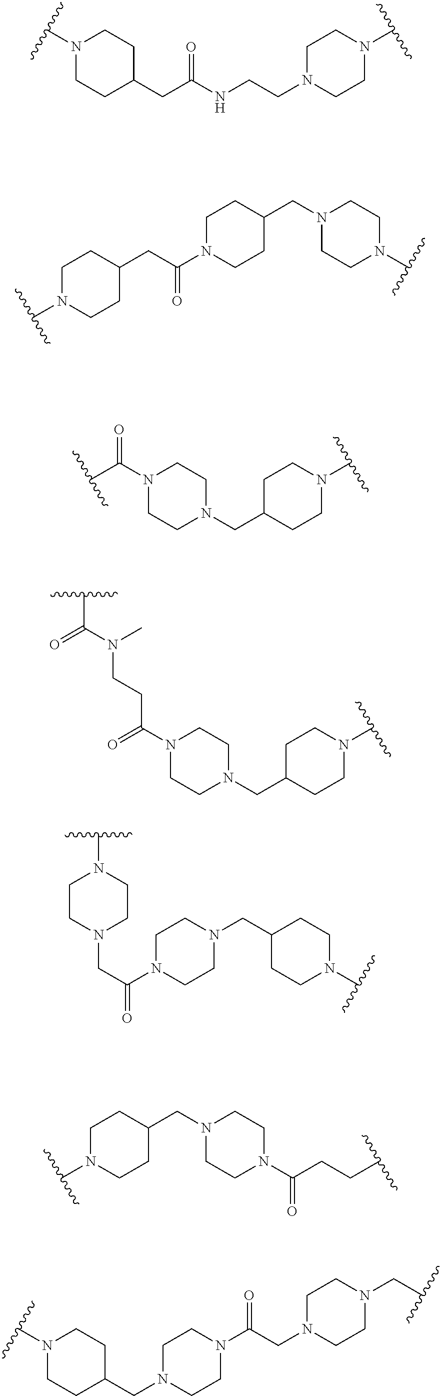

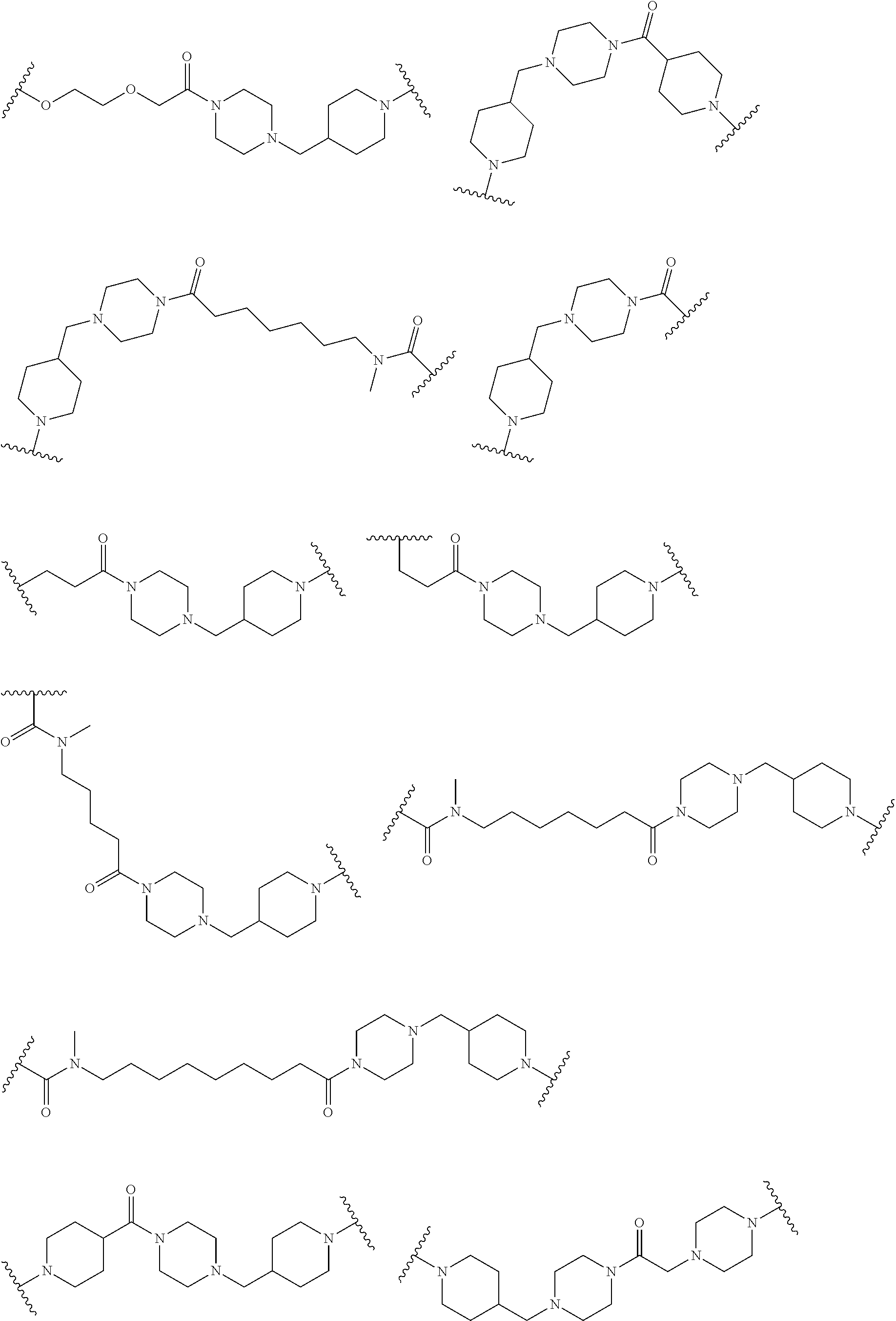

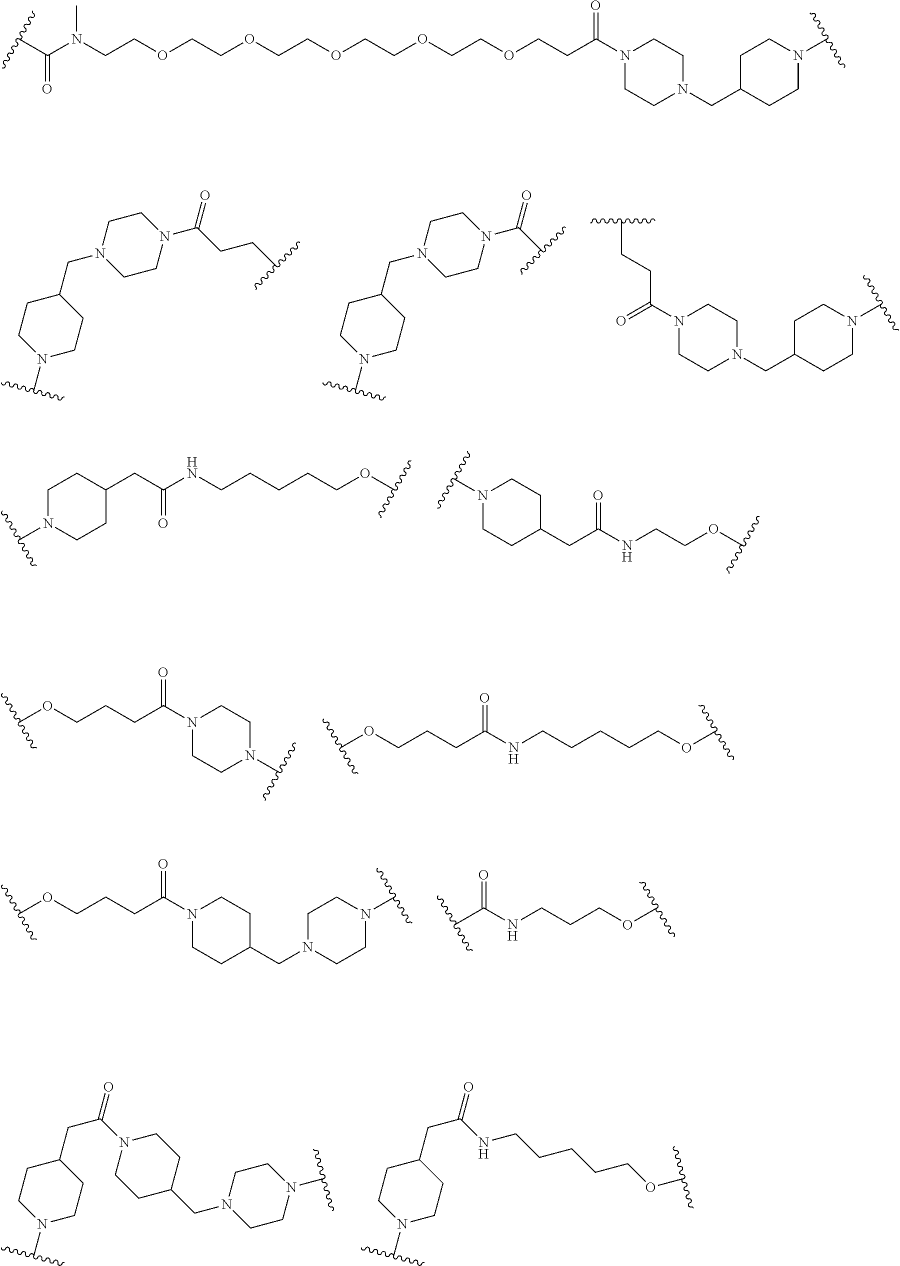

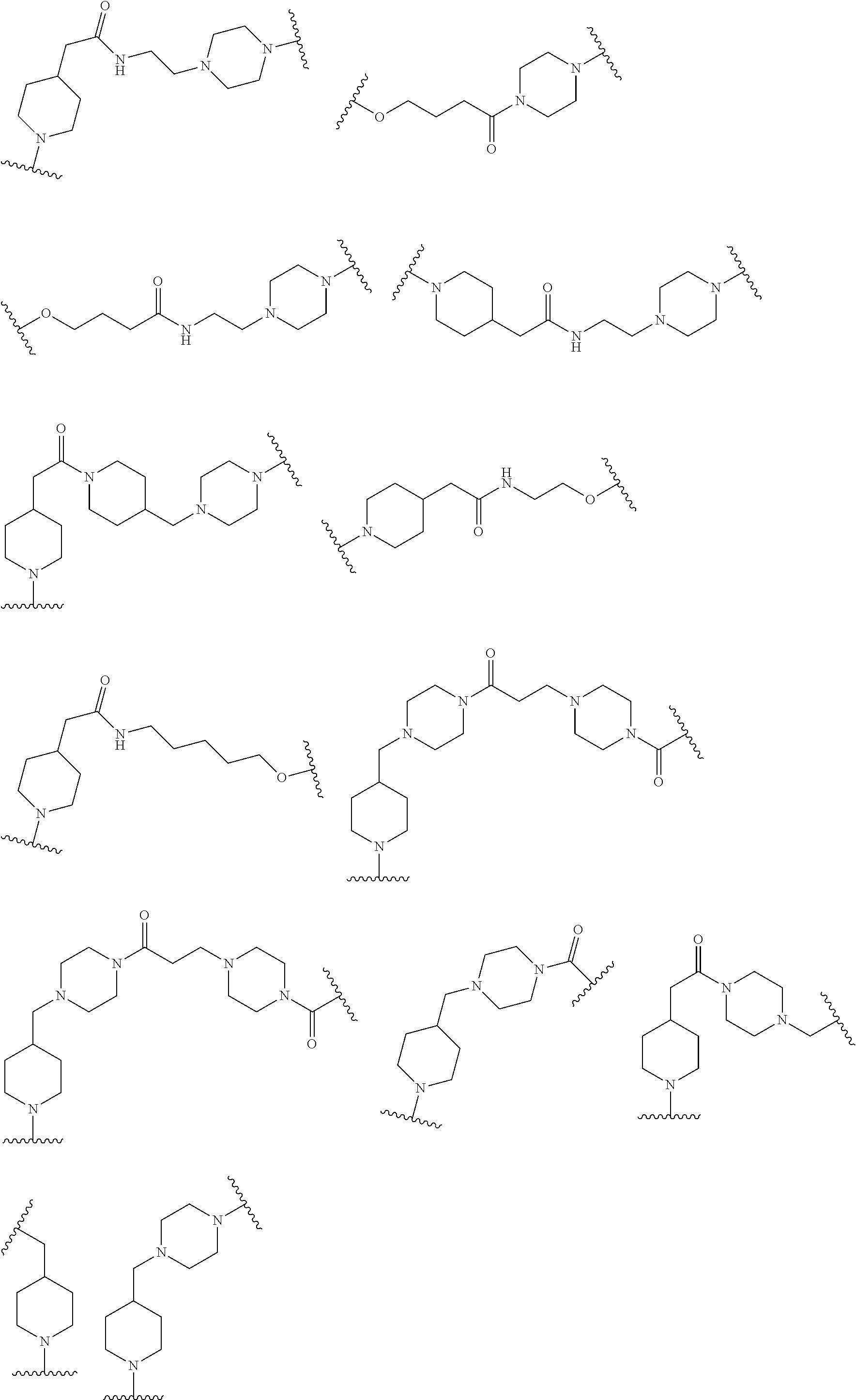

In some cases, the structures shown above represent the entire linker. In other examples, the linker of the bifunctional molecule may comprise a plurality of the structures shown above.

In these structures, the wavy lines are shown over the bond(s) that forms the link with the TBL and Z moieties respectively.

In some examples, the bond(s) that forms the link with the TBL and/or Z moieties is (are) attached to a ring structure. On many of the structures described herein, this bond is shown as being attached at a particular position on the ring structure. However, the disclosure also encompasses joining or coupling to the TBL and Z moieties at any chemically suitable position on these ring structures.

The present disclosure encompasses the use of any of the linkers disclosed herein in combination with any of the Z moieties and TBL moieties described herein.

In particular examples, the linker may not be:

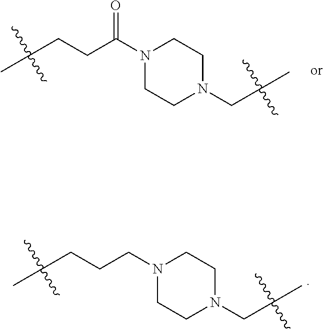 [SMILES: *CCC(=O)N1CCN(C*)CC1 ; *CCCN1CCN(C*)CC1]

In more particular examples, the bifunctional molecule may not comprise:

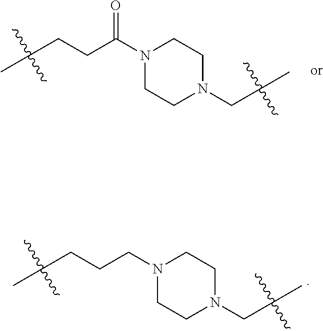 [SMILES: *CCC(=O)N1CCN(C*)CC1 ; *CCCN1CCN(C*)CC1]

In other cases, the linker may not be:

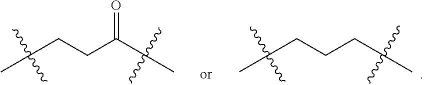 [SMILES: *CCC(*)=O ; *CCC*]

In some examples, the bifunctional molecule comprising the general formula TBL-L-Z may be selected from any of the following:

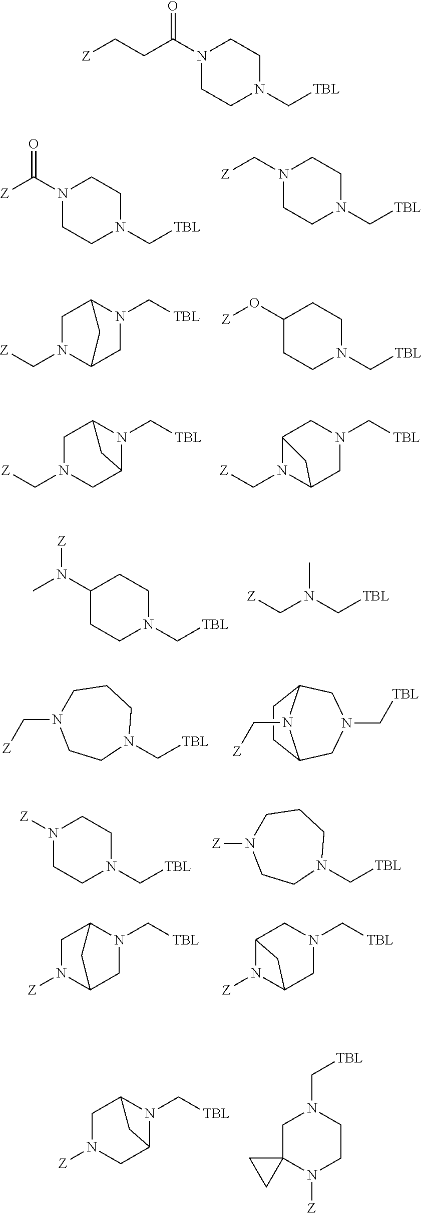

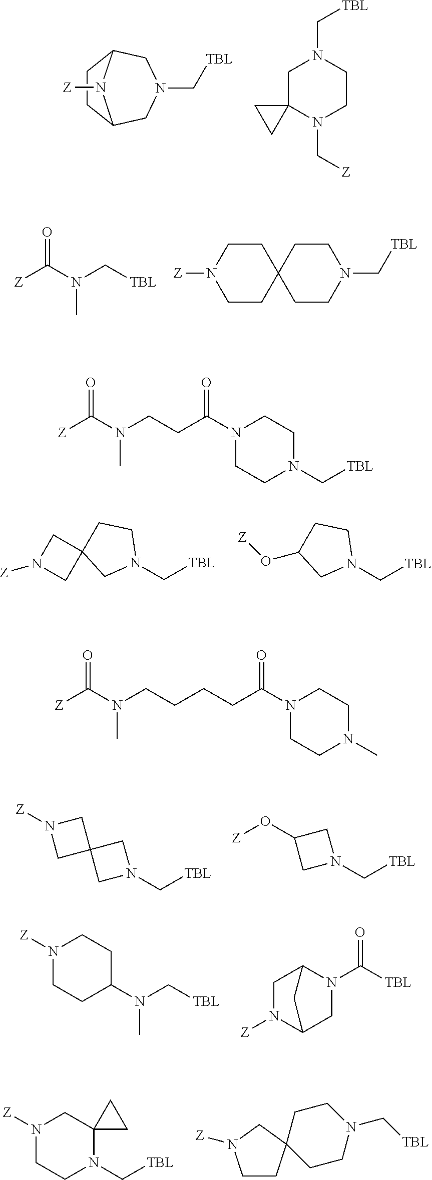

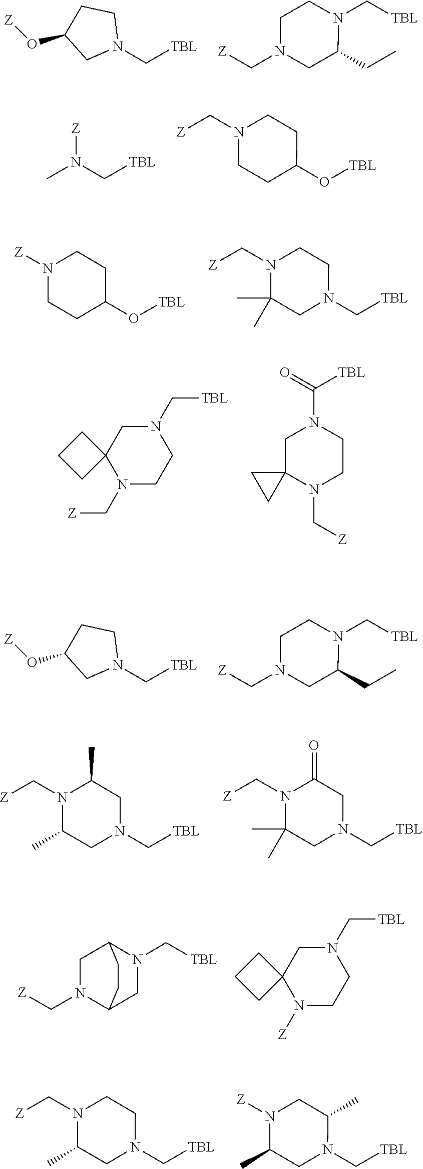

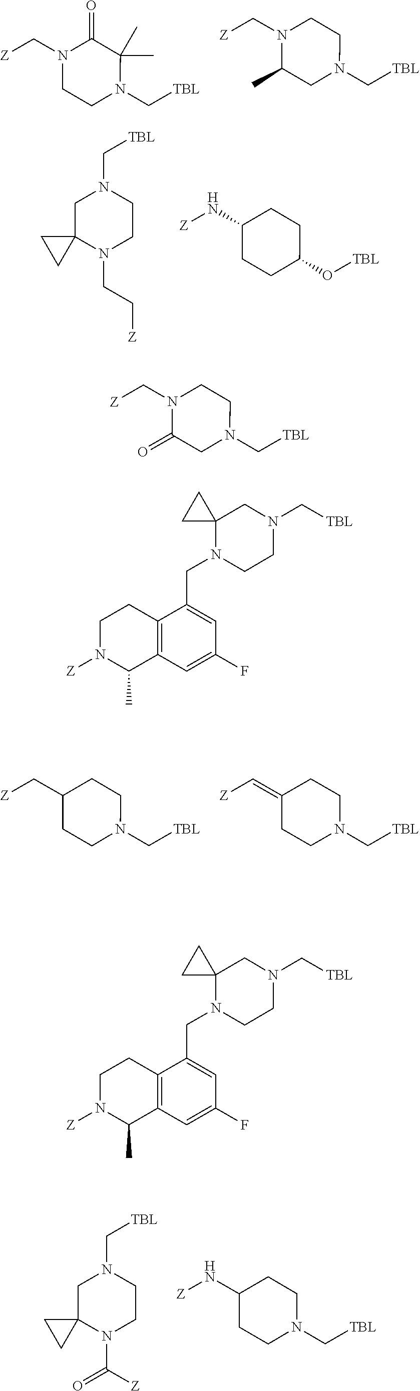

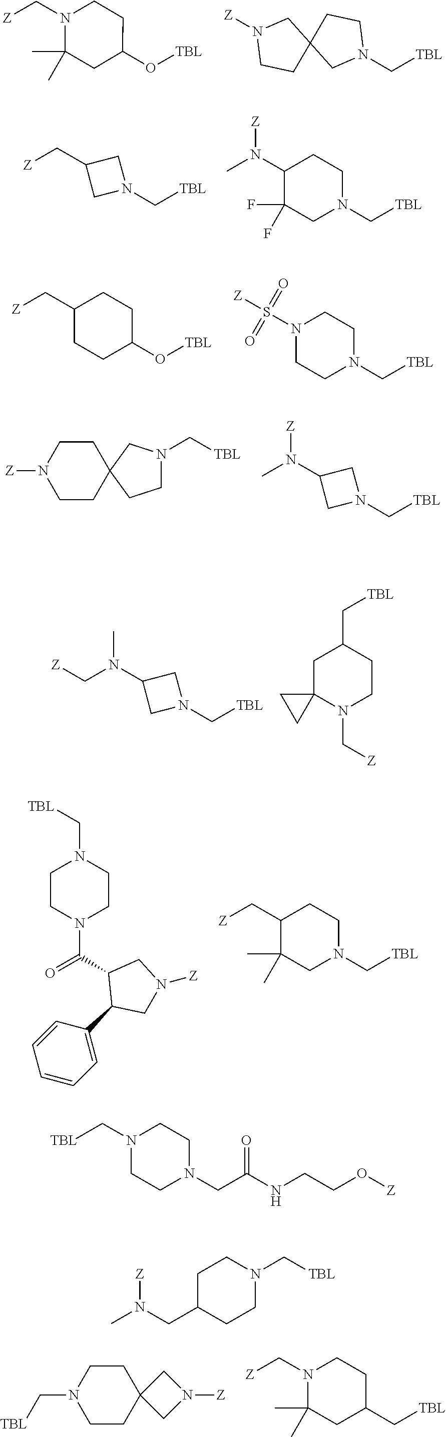

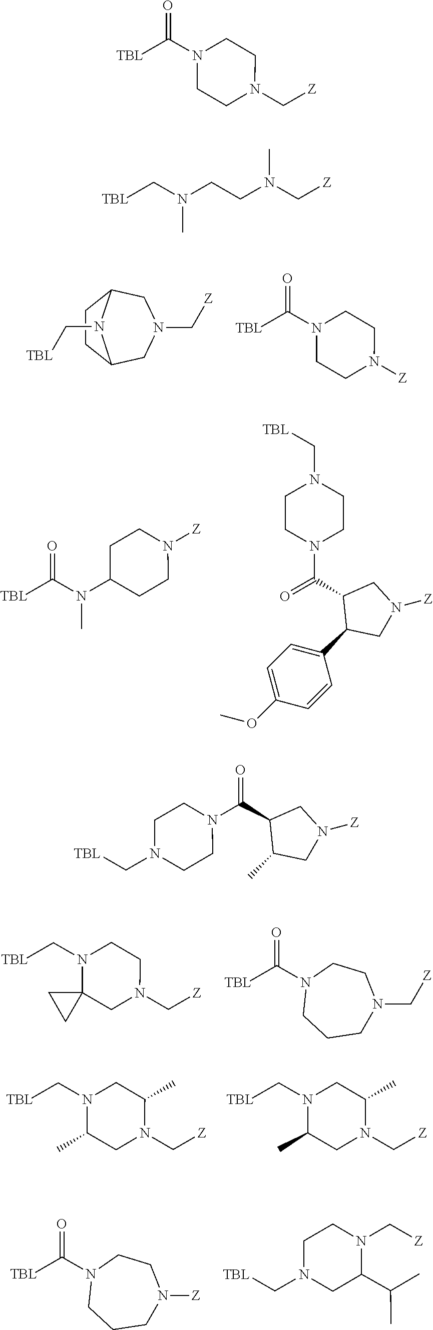

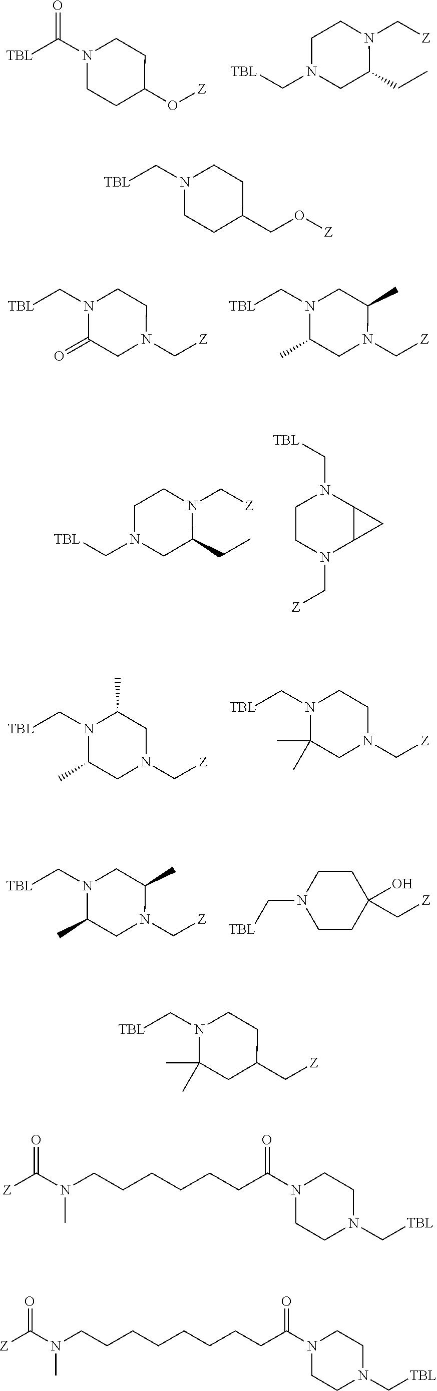

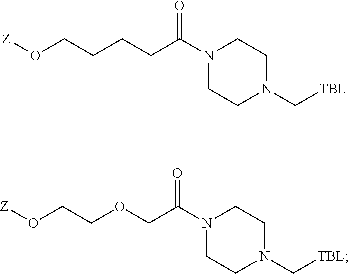

wherein Z and TBL are as defined above and herein.

In some examples, the bifunctional molecule comprising the general formula TBL-L-Z comprises one of the following structures:

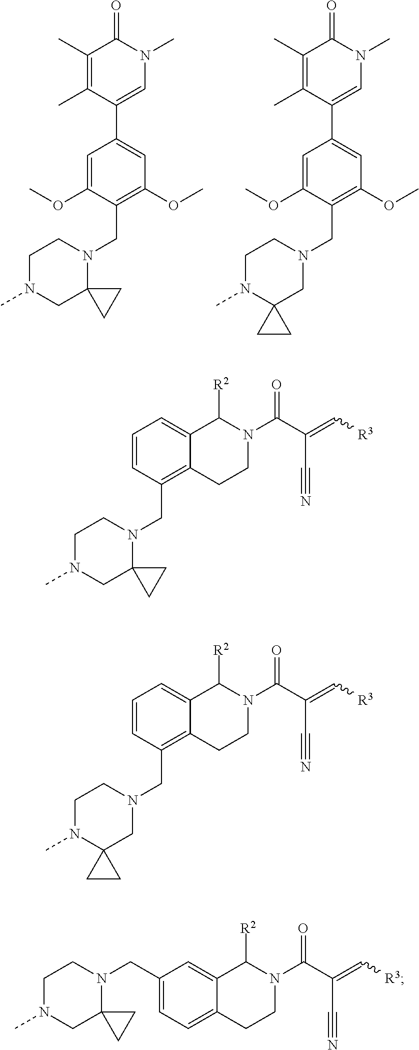

wherein R² and R³ are as defined above and herein, and the bond indicates the linkage to the rest of the bifunctional molecule.

### Exemplary Bifunctional Molecules

It will be appreciated that the bifunctional molecules of the present disclosure may exist in different stereoisomeric forms. The present disclosure includes within its scope the use of all stereoisomeric forms, or the use of a mixture of stereoisomers of the bifunctional molecules, By way of example, where the bifunctional molecule comprises one or more chiral centres, the present disclosure encompasses each individual enantiomer of the bifunctional molecule as well as mixtures of enantiomers including racemic mixtures of such enantiomers. By way of further example, where the bifunctional molecule comprises two or more chiral centres, the present disclosure encompasses each individual diastereomer of the bifunctional molecule, as well as mixtures of the various diastereomers.

Unless otherwise indicated, the various structures shown herein encompass all isomeric (e.g. enantiomeric, diastereomeric, and geometric (or conformational)) forms of the structure). For example, the present disclosure embraces the R and S configurations for each asymmetric centre, and Z and E double bond isomers. Therefore, single stereochemical isomers as well as enantiomeric, diastereomeric, and geometric (or conformational) mixtures of the present compounds are to be understood to be within the scope of the present disclosure. Additionally, unless otherwise stated, where present, all tautomeric forms of the bifunctional molecules described herein are to be understood to be within the scope of the present disclosure.

As used herein, references to “a bifunctional molecule” may further embrace a pharmaceutically acceptable salt thereof.

For the avoidance of doubt, the bifunctional molecule may comprise any combination of target binding protein (TBL), linker (L) and warhead (Z) (provided that it has the correct valency and/or is chemically suitable). For example, the bifunctional compound may comprise any combination of Z of formula (I), (II) or (III) (inc. corresponding subgeneric formulae defined herein, such as (Ia), (Ib), (Ic), (IIa), (IIaa), (IIb), (IIc), and (IId)), L of any formula or subgeneric formula defined herein, and TBL of or comprising formula 1a, 1a′, 1b, 1c, 1b′, 1a¹, 1a², 1a³, 1e, 1f, 1g, 1f′, 1g′, 1ea to 1eh, 1fa to 1fh, 1ga, 1ea′, 1h to 1z or 2a to 2g. In other examples, the bifunctional compound comprises any combination of Z of formula (WZI) to (WZV), (WI), (WII), (WIII), (WIV) or (WV) (inc. corresponding subgeneric formulae defined herein, such as (WIa) to (WIf), (WIIa) to (WIIf), (WIIIa), (WIIIb) and (WIVa), (A), (A1) to (A3), L and TBL of any formula or subgeneric formula defined herein.

In some embodiments:
(i) Z is represented as formula (I), (Ia), (Ib), (Ic), (IIaa), (IIa) or (IIb) as defined above; and
(ii) TBL is represented by formula (1e), (1f) or (1f′) as defined above.

In other embodiments:
(i) Z is represented as formula (Ia), (IIaa), or (IIa) as defined above; and
(ii) TBL is represented by formula (1e), (1f) or (1f′) as defined above.

In particular embodiments:
(i) Z is represented as formula (Ia), (IIaa), or (IIa) as defined above; and
(ii) TBL is represented by formula (1h), (1i) or (1j) as defined above.

In other particular embodiments:
(i) Z is represented as formula (Ib), or (IIb) as defined above; and
(ii) TBL is represented by formula (1e), (1f) or (1f′) as defined above.

In yet more particular embodiments:
(i) Z is represented as formula (Ib), or (IIb) as defined above; and
(ii) TBL is represented by formula (1h), (1i) or (1j) as defined above.

In certain embodiments:
(i) Z is represented as formula (Ia), (IIaa) or (IIa) as defined above;
(ii) TBL is represented by formula 1a″ as defined above.

In these specific embodiments, L may be represented by formula L1a or L1b.

In yet further embodiments:
(i) Z is represented as formula (Ia), (IIaa) or (IIa) as defined above;
(ii) TBL is represented by any one of formulae 1e″, 1g″, 1g′″, 1ea″ to 1eh″, 1ea″, 1h″ to 1z″ and 2a″ to 2g″ as defined above; and
(iii) L is represented by formula L1a or L1b as defined above.

In even more particular embodiments:
(i) Z is represented as formula (WI), (WII), (WIIa), (WIIb), (WIIc), (WIId), (WIIe), (WIIf), (WIII), (WIIIa), (WIIIb), (WIV) or (WIVa) as defined above; and
(ii) TBL is represented by formula (1e), (1f) or (1f′) as defined above.

In some examples:
(i) Z is represented as formula (WI), (WII), (WIIa), (WIIb), (WIIc), (WIId), (WIIe), (WIIf), (WIII), (WIIIa), (WIIIb), (WIV) or (WIVa) as defined above;
   wherein Z is not:

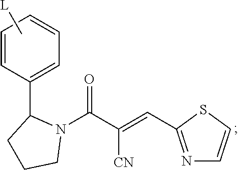

and
(ii) TBL is the target protein binding ligand that binds BRD9, wherein TBL is not

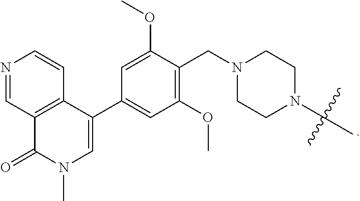 [SMILES: *N1CCN(Cc2c(OC)cc(-c3cn(C)c(=O)c4cnccc34)cc2OC)CC1]

In some embodiments:
(i) Z is represented as any one of formula (WZI), (WZII), (WZIIa) to (WZIIe), (WZIIIa) to (WZIIIh) or (WZIVa) to (WZIVj) as defined above;
(ii) TBL is represented by formula 1a″ as defined above; and
(iii) L is represented by formula L1c as defined above.

In some embodiments:
(i) Z is represented as any one of formula (WZI), (WZII), (WZIIa) to (WZIIe), (WZIIIa) to (WZIIIh) or (WZIVa) to (WZIVj) as defined above;
(ii) TBL is represented by any one of formulae 1e″, 1g″, 1g′″, 1ea″ to 1eh″, 1ea″, 1h″ to 1z″ and 2a″ to 2g″ as defined above; and
(iii) L is represented by formula L1c as defined above.

In some cases, the bifunctional molecule is not:

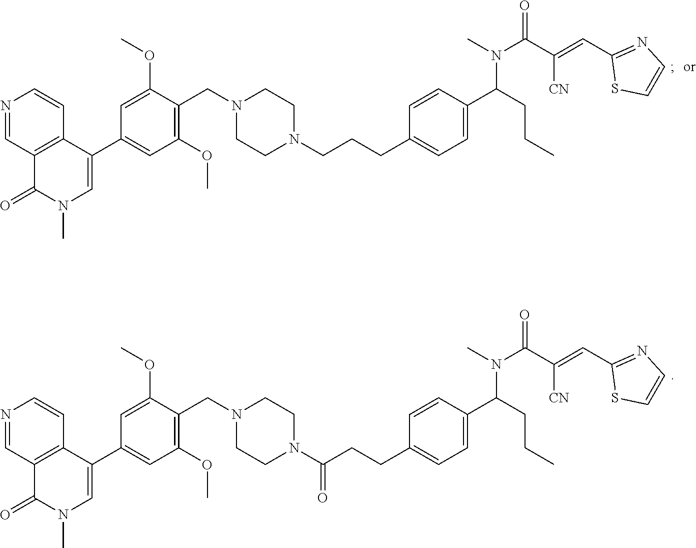 [SMILES: CCCC(c1ccc(CCCN2CCN(Cc3c(OC)cc(-c4cn(C)c(=O)c5cnccc45)cc3OC)CC2)cc1)N(C)C(=O)/C(C#N)=C/c1nccs1 ; CCCC(c1ccc(CCC(=O)N2CCN(Cc3c(OC)cc(-c4cn(C)c(=O)c5cnccc45)cc3OC)CC2)cc1)N(C)C(=O)/C(C#N)=C/c1nccs1]

In some more specific examples, the bifunctional molecule is any one of formulae A2 to A76, BRD9a to BRD9ac, B1 to B84, B86, B88 to B96, B98 to B104, B106 to B127, B130 to B149, B152 to B156, B158 to B162, B164, B165, B169, B173 to B175, B180 to B215, and C1 to C107 or any combination of TBL, L and Z represented in A2 to A76, BRD9a to BRD9ac, B1 to B84, B86, B88 to B96, B98 to B104, B106 to B127, B130 to B149, B152 to B156, B158 to B162, B164, B165, B169, B173 to B175, B180 to B215, and C1 to C107 as shown in Table 1 below:

| | |
|---|---|
| 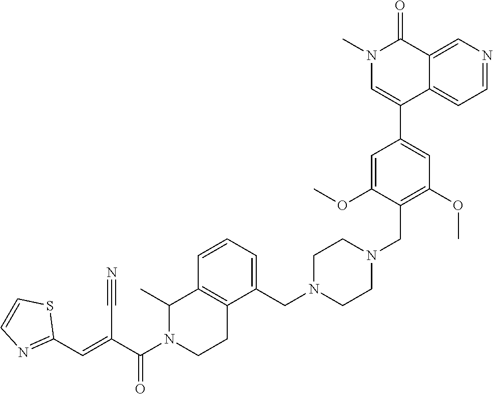 [SMILES: COc1cc(-c2cn(C)c(=O)c3cnccc23)cc(OC)c1CN1CCN(Cc2cccc3c2CCN(C(=O)/C(C#N)=C/c2nccs2)C3C)CC1] | A2 |
| 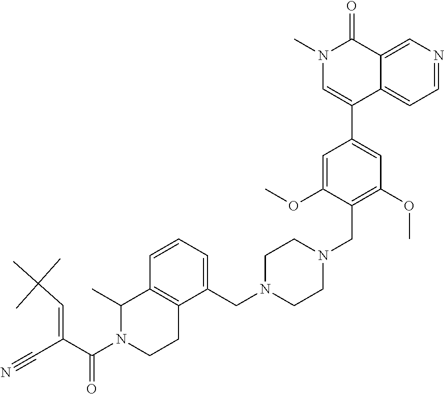 [SMILES: COc1cc(-c2cn(C)c(=O)c3cnccc23)cc(OC)c1CN1CCN(Cc2cccc3c2CCN(C(=O)/C(C#N)=C/C(C)(C)C)C3C)CC1] | A3 |
| 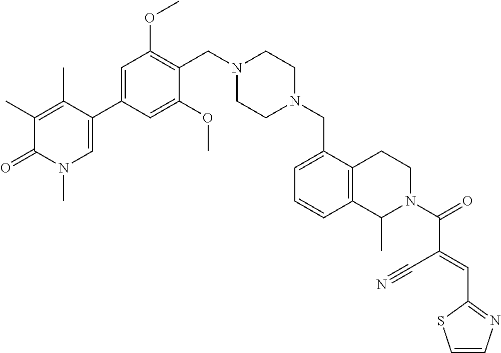 [SMILES: COc1cc(-c2cn(C)c(=O)c(C)c2C)cc(OC)c1CN1CCN(Cc2cccc3c2CCN(C(=O)/C(C#N)=C/c2nccs2)C3C)CC1] | A4 |
| 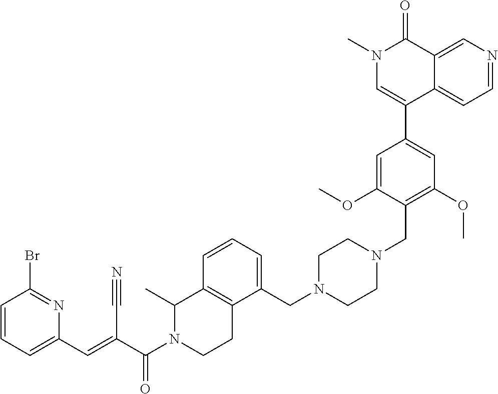 [SMILES: COc1cc(-c2cn(C)c(=O)c3cnccc23)cc(OC)c1CN1CCN(Cc2cccc3c2CCN(C(=O)/C(C#N)=C/c2cccc(Br)n2)C3C)CC1] | A5 |
| 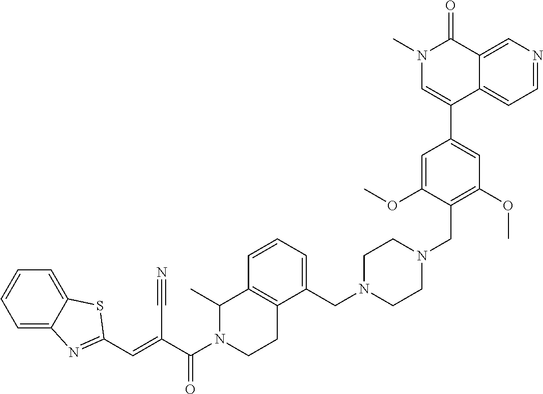 [SMILES: COc1cc(-c2cn(C)c(=O)c3cnccc23)cc(OC)c1CN1CCN(Cc2cccc3c2CCN(C(=O)/C(C#N)=C/c2nc4ccccc4s2)C3C)CC1] | A6 |
| 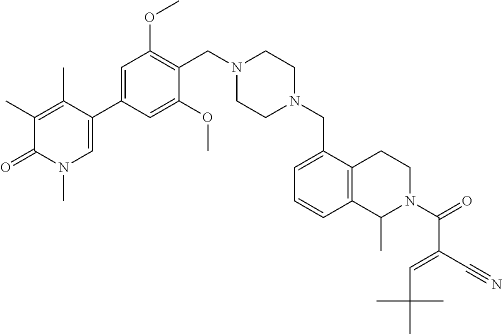 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)CC1] | A7 |
| 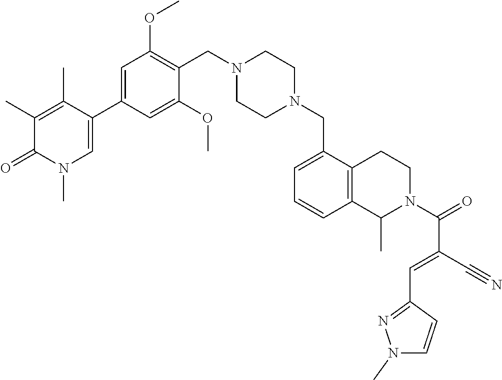 [SMILES: COc1cc(-c2cn(C)c(=O)c(C)c2C)cc(OC)c1CN1CCN(Cc2cccc3c2CCN(C(=O)/C(C#N)=C/c2ccn(C)n2)C3C)CC1] | A8 |
| 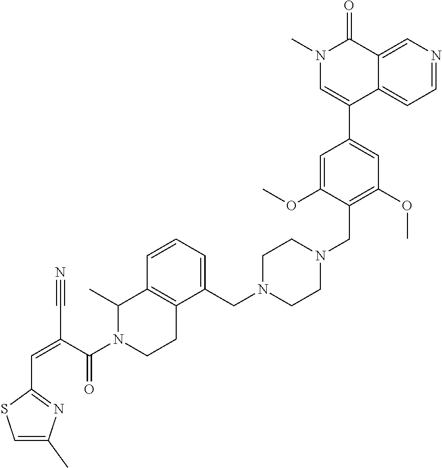 [SMILES: COc1cc(-c2cn(C)c(=O)c3cnccc23)cc(OC)c1CN1CCN(Cc2cccc3c2CCN(C(=O)/C(C#N)=C\c2nc(C)cs2)C3C)CC1] | A9 |
| 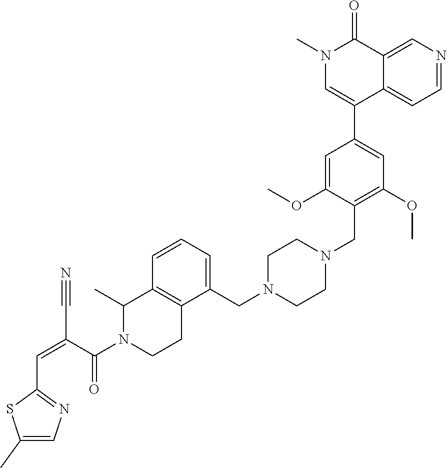 [SMILES: COc1cc(-c2cn(C)c(=O)c3cnccc23)cc(OC)c1CN1CCN(Cc2cccc3c2CCN(C(=O)/C(C#N)=C\c2ncc(C)s2)C3C)CC1] | A10 |
| 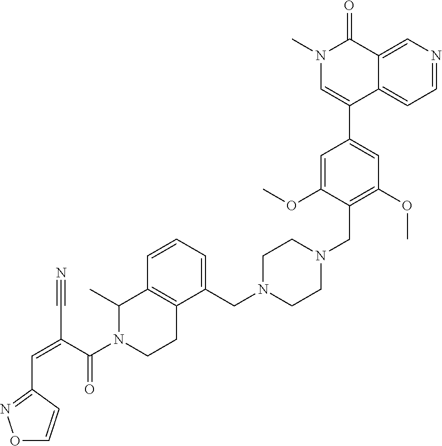 [SMILES: COc1cc(-c2cn(C)c(=O)c3cnccc23)cc(OC)c1CN1CCN(Cc2cccc3c2CCN(C(=O)/C(C#N)=C\c2ccon2)C3C)CC1] | A11 |
| 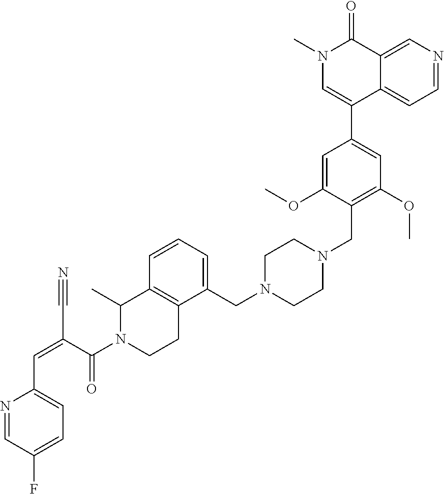 [SMILES: COc1cc(-c2cn(C)c(=O)c3cnccc23)cc(OC)c1CN1CCN(Cc2cccc3c2CCN(C(=O)/C(C#N)=C\c2ccc(F)cn2)C3C)CC1] | A12 |
| 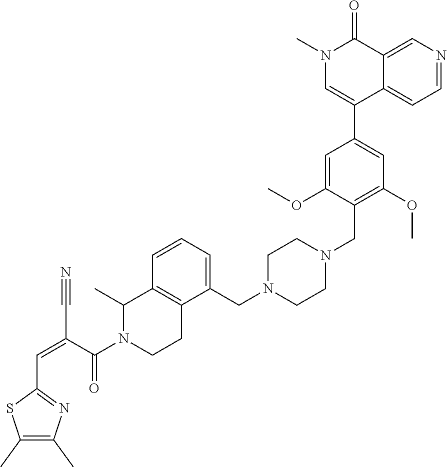 [SMILES: COc1cc(-c2cn(C)c(=O)c3cnccc23)cc(OC)c1CN1CCN(Cc2cccc3c2CCN(C(=O)/C(C#N)=C\c2nc(C)c(C)s2)C3C)CC1] | A13 |
| 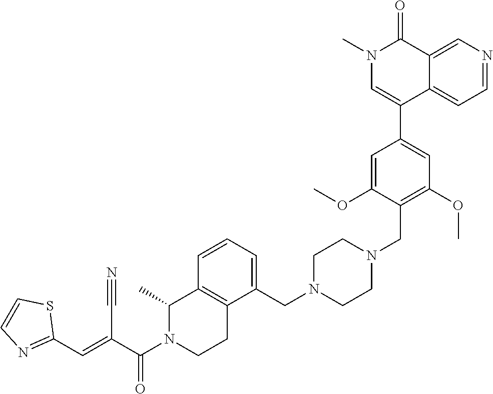 [SMILES: COc1cc(-c2cn(C)c(=O)c3cnccc23)cc(OC)c1CN1CCN(Cc2cccc3c2CCN(C(=O)/C(C#N)=C/c2nccs2)[C@@H]3C)CC1] | A14 |
| 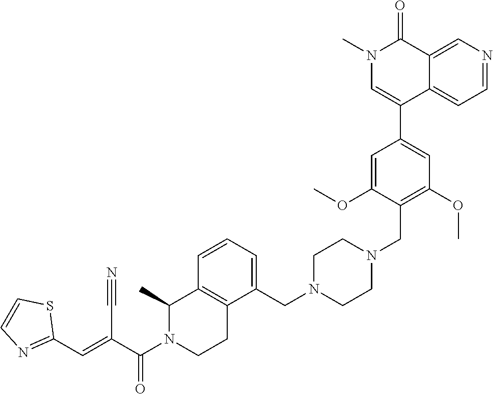 [SMILES: COc1cc(-c2cn(C)c(=O)c3cnccc23)cc(OC)c1CN1CCN(Cc2cccc3c2CCN(C(=O)/C(C#N)=C/c2nccs2)[C@H]3C)CC1] | A15 |
| 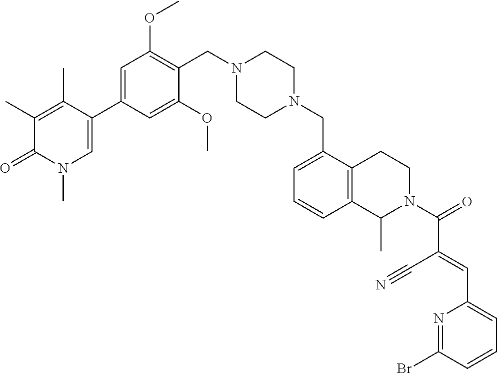 [SMILES: COc1cc(-c2cn(C)c(=O)c(C)c2C)cc(OC)c1CN1CCN(Cc2cccc3c2CCN(C(=O)/C(C#N)=C/c2cccc(Br)n2)C3C)CC1] | A16 |
| 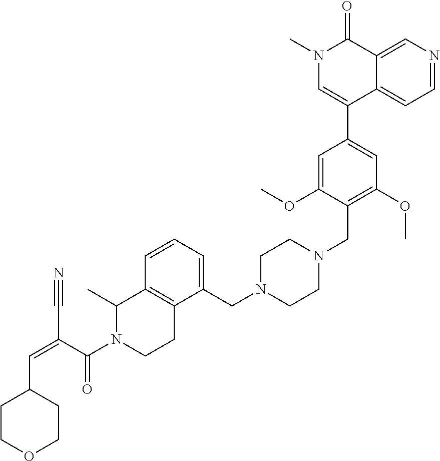 [SMILES: COc1cc(-c2cn(C)c(=O)c3cnccc23)cc(OC)c1CN1CCN(Cc2cccc3c2CCN(C(=O)/C(C#N)=C\C2CCOCC2)C3C)CC1] | A17 |
| 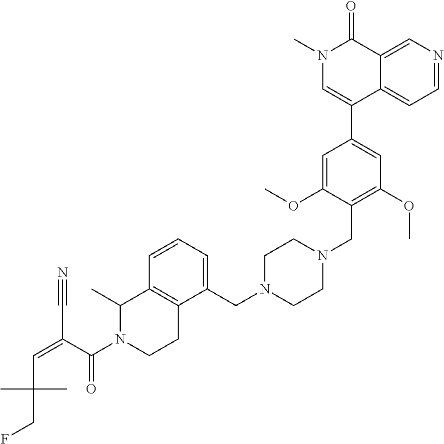 [SMILES: COc1cc(-c2cn(C)c(=O)c3cnccc23)cc(OC)c1CN1CCN(Cc2cccc3c2CCN(C(=O)/C(C#N)=C\C(C)(C)CF)C3C)CC1] | A18 |
| 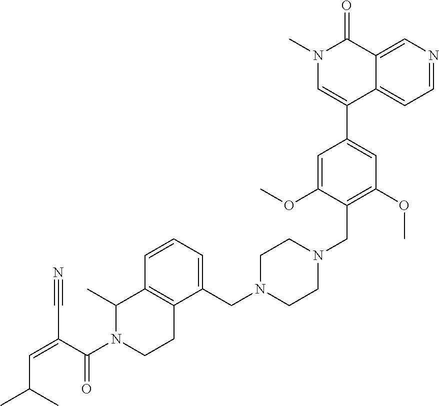 [SMILES: COc1cc(-c2cn(C)c(=O)c3cnccc23)cc(OC)c1CN1CCN(Cc2cccc3c2CCN(C(=O)/C(C#N)=C\C(C)C)C3C)CC1] | A19 |
| 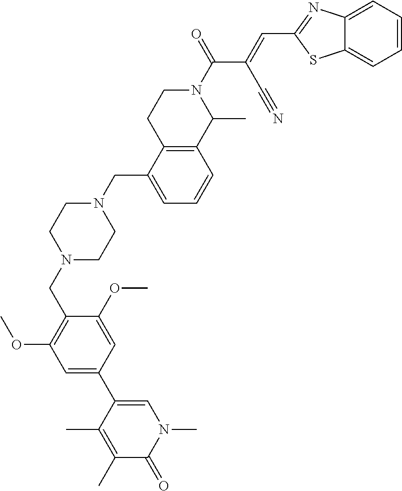 [SMILES: COc1cc(-c2cn(C)c(=O)c(C)c2C)cc(OC)c1CN1CCN(Cc2cccc3c2CCN(C(=O)/C(C#N)=C/c2nc4ccccc4s2)C3C)CC1] | A20 |
| 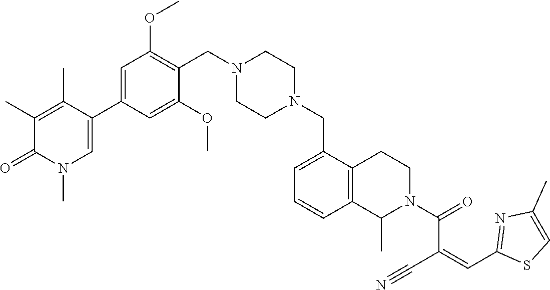 [SMILES: COc1cc(-c2cn(C)c(=O)c(C)c2C)cc(OC)c1CN1CCN(Cc2cccc3c2CCN(C(=O)/C(C#N)=C\c2nc(C)cs2)C3C)CC1] | A21 |
| 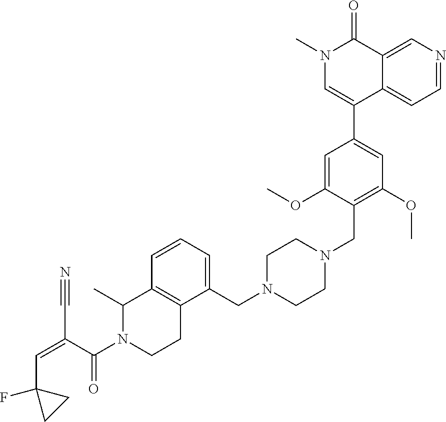 [SMILES: COc1cc(-c2cn(C)c(=O)c3cnccc23)cc(OC)c1CN1CCN(Cc2cccc3c2CCN(C(=O)/C(C#N)=C\C2(F)CC2)C3C)CC1] | A22 |
| 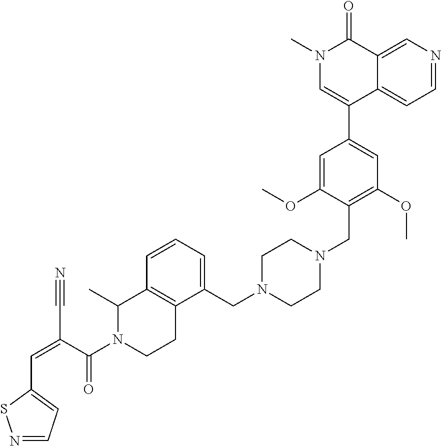 [SMILES: COc1cc(-c2cn(C)c(=O)c3cnccc23)cc(OC)c1CN1CCN(Cc2cccc3c2CCN(C(=O)/C(C#N)=C\c2ccns2)C3C)CC1] | A23 |
| 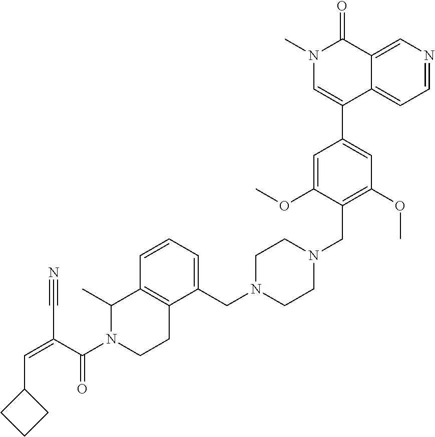 [SMILES: COc1cc(-c2cn(C)c(=O)c3cnccc23)cc(OC)c1CN1CCN(Cc2cccc3c2CCN(C(=O)/C(C#N)=C\C2CCC2)C3C)CC1] | A24 |
| 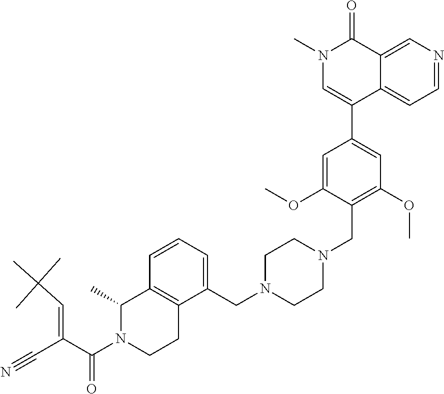 [SMILES: COc1cc(-c2cn(C)c(=O)c3cnccc23)cc(OC)c1CN1CCN(Cc2cccc3c2CCN(C(=O)/C(C#N)=C/C(C)(C)C)[C@@H]3C)CC1] | A25 |
| 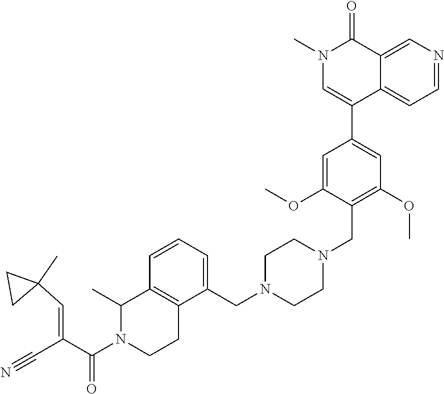 [SMILES: COc1cc(-c2cn(C)c(=O)c3cnccc23)cc(OC)c1CN1CCN(Cc2cccc3c2CCN(C(=O)/C(C#N)=C/C2(C)CC2)C3C)CC1] | A26 |
| 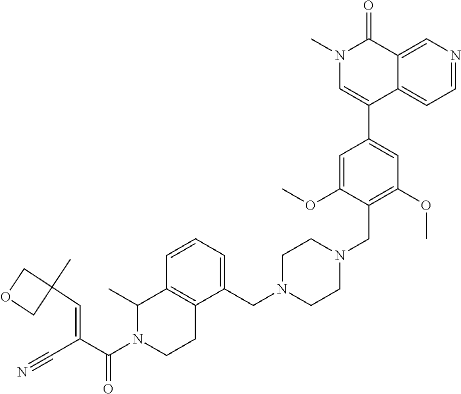 [SMILES: COc1cc(-c2cn(C)c(=O)c3cnccc23)cc(OC)c1CN1CCN(Cc2cccc3c2CCN(C(=O)/C(C#N)=C/C2(C)COC2)C3C)CC1] | A27 |
| 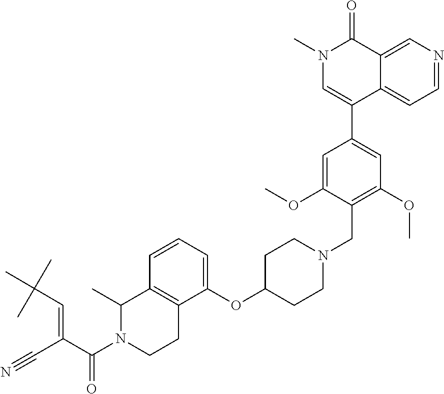 [SMILES: COc1cc(-c2cn(C)c(=O)c3cnccc23)cc(OC)c1CN1CCC(Oc2cccc3c2CCN(C(=O)/C(C#N)=C/C(C)(C)C)C3C)CC1] | A28 |
| 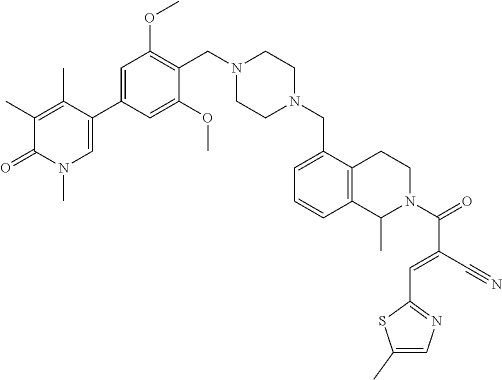 [SMILES: COc1cc(-c2cn(C)c(=O)c(C)c2C)cc(OC)c1CN1CCN(Cc2cccc3c2CCN(C(=O)/C(C#N)=C/c2ncc(C)s2)C3C)CC1] | A29 |
| 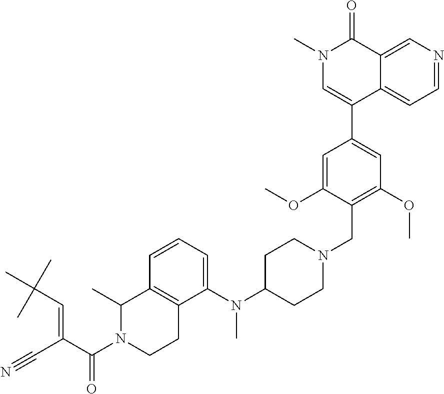 [SMILES: COc1cc(-c2cn(C)c(=O)c3cnccc23)cc(OC)c1CN1CCC(N(C)c2cccc3c2CCN(C(=O)/C(C#N)=C/C(C)(C)C)C3C)CC1] | A30 |
| 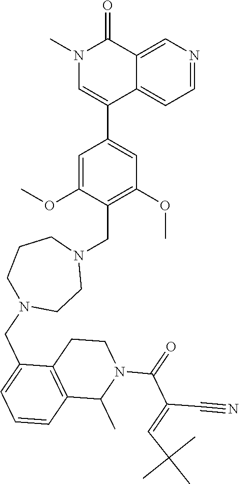 [SMILES: COc1cc(-c2cn(C)c(=O)c3cnccc23)cc(OC)c1CN1CCCN(Cc2cccc3c2CCN(C(=O)/C(C#N)=C/C(C)(C)C)C3C)CC1] | A31 |
| 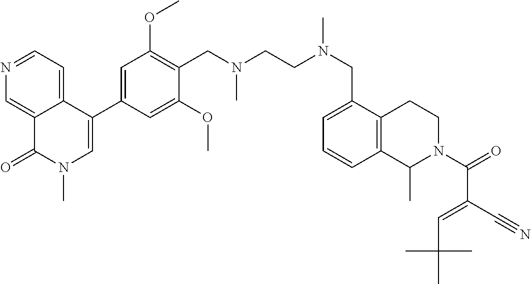 [SMILES: COc1cc(-c2cn(C)c(=O)c3cnccc23)cc(OC)c1CN(C)CCN(C)Cc1cccc2c1CCN(C(=O)/C(C#N)=C/C(C)(C)C)C2C] | A32 |
| 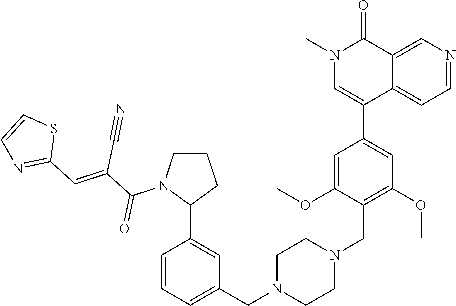 [SMILES: COc1cc(-c2cn(C)c(=O)c3cnccc23)cc(OC)c1CN1CCN(Cc2cccc(C3CCCN3C(=O)/C(C#N)=C/c3nccs3)c2)CC1] | A33 |
| 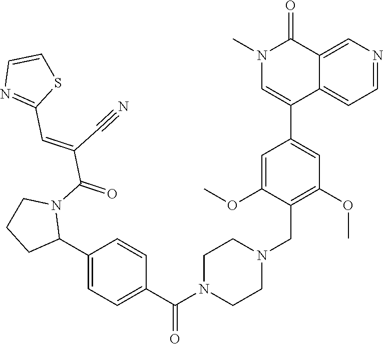 [SMILES: COc1cc(-c2cn(C)c(=O)c3cnccc23)cc(OC)c1CN1CCN(C(=O)c2ccc(C3CCCN3C(=O)/C(C#N)=C/c3nccs3)cc2)CC1] | A34 |
| 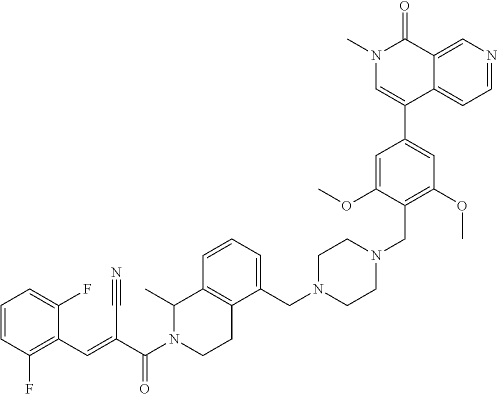 [SMILES: COc1cc(-c2cn(C)c(=O)c3cnccc23)cc(OC)c1CN1CCN(Cc2cccc3c2CCN(C(=O)/C(C#N)=C/c2c(F)cccc2F)C3C)CC1] | A35 |
| 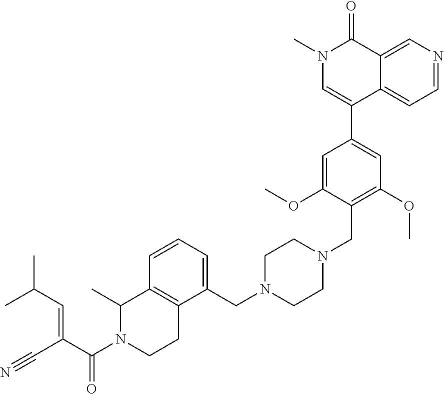 [SMILES: COc1cc(-c2cn(C)c(=O)c3cnccc23)cc(OC)c1CN1CCN(Cc2cccc3c2CCN(C(=O)/C(C#N)=C/C(C)C)C3C)CC1] | A36 |
| 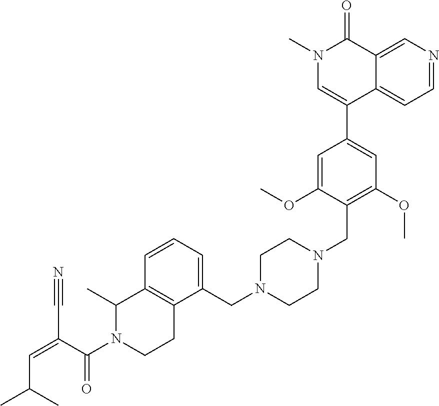 [SMILES: COc1cc(-c2cn(C)c(=O)c3cnccc23)cc(OC)c1CN1CCN(Cc2cccc3c2CCN(C(=O)/C(C#N)=C\C(C)C)C3C)CC1] | A37 |
| 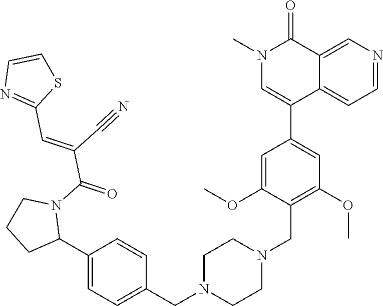 [SMILES: COc1cc(-c2cn(C)c(=O)c3cnccc23)cc(OC)c1CN1CCN(Cc2ccc(C3CCCN3C(=O)/C(C#N)=C/c3nccs3)cc2)CC1] | A38 |
| 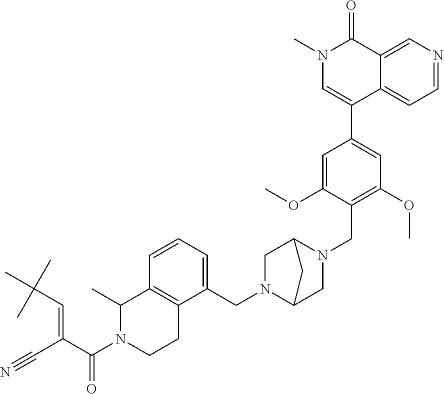 [SMILES: COc1cc(-c2cn(C)c(=O)c3cnccc23)cc(OC)c1CN1CC2CC1CN2Cc1cccc2c1CCN(C(=O)/C(C#N)=C/C(C)(C)C)C2C] | A39 |
| 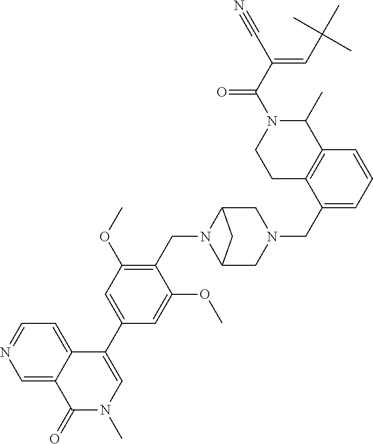 [SMILES: COc1cc(-c2cn(C)c(=O)c3cnccc23)cc(OC)c1CN1C2CC1CN(Cc1cccc3c1CCN(C(=O)/C(C#N)=C/C(C)(C)C)C3C)C2] | A40 |
| 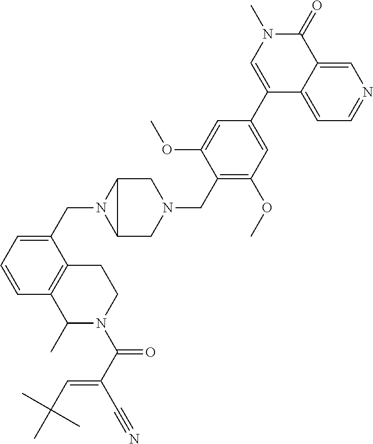 [SMILES: COc1cc(-c2cn(C)c(=O)c3cnccc23)cc(OC)c1CN1CC2C(C1)N2Cc1cccc2c1CCN(C(=O)/C(C#N)=C/C(C)(C)C)C2C] | A41 |
| 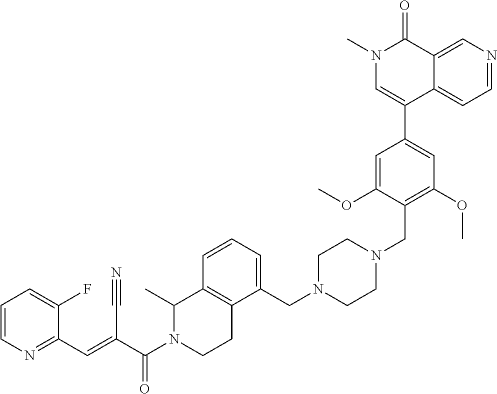 [SMILES: COc1cc(-c2cn(C)c(=O)c3cnccc23)cc(OC)c1CN1CCN(Cc2cccc3c2CCN(C(=O)/C(C#N)=C/c2ncccc2F)C3C)CC1] | A42 |
| 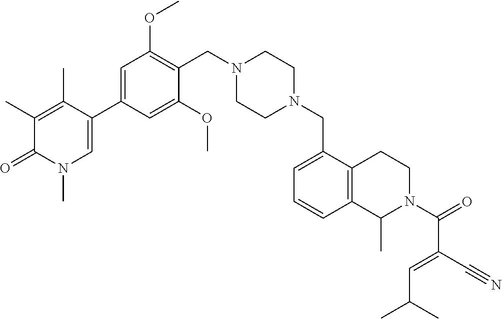 [SMILES: COc1cc(-c2cn(C)c(=O)c(C)c2C)cc(OC)c1CN1CCN(Cc2cccc3c2CCN(C(=O)/C(C#N)=C/C(C)C)C3C)CC1] | A43 |
| 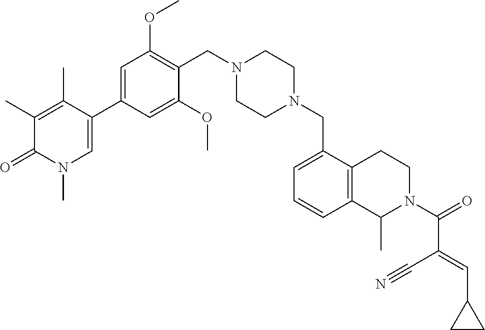 [SMILES: COc1cc(-c2cn(C)c(=O)c(C)c2C)cc(OC)c1CN1CCN(Cc2cccc3c2CCN(C(=O)/C(C#N)=C/C2CC2)C3C)CC1] | A44 |
| 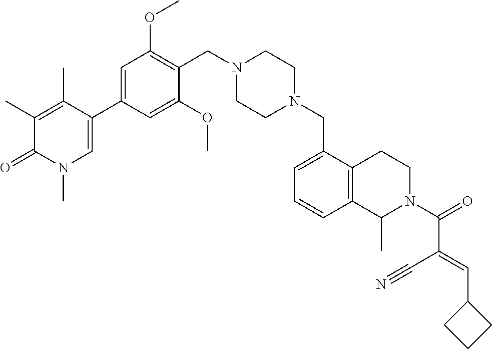 [SMILES: COc1cc(-c2cn(C)c(=O)c(C)c2C)cc(OC)c1CN1CCN(Cc2cccc3c2CCN(C(=O)/C(C#N)=C/C2CCC2)C3C)CC1] | A45 |
| 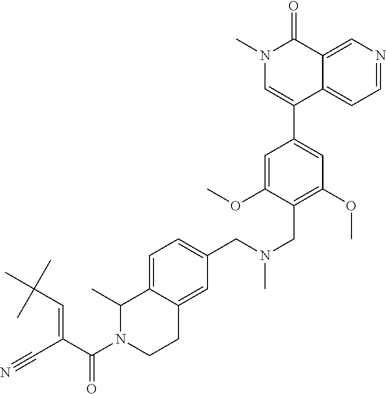 [SMILES: COc1cc(-c2cn(C)c(=O)c3cnccc23)cc(OC)c1CN(C)Cc1ccc2c(c1)CCN(C(=O)/C(C#N)=C/C(C)(C)C)C2C] | A46 |
| 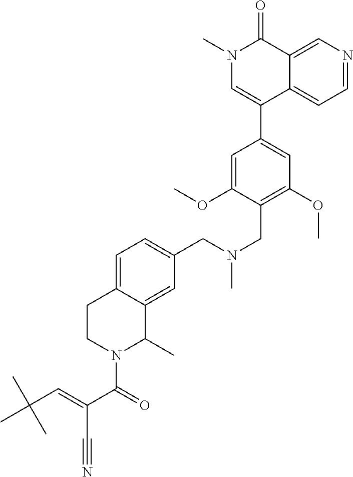 [SMILES: COc1cc(-c2cn(C)c(=O)c3cnccc23)cc(OC)c1CN(C)Cc1ccc2c(c1)C(C)N(C(=O)/C(C#N)=C/C(C)(C)C)CC2] | A47 |
| 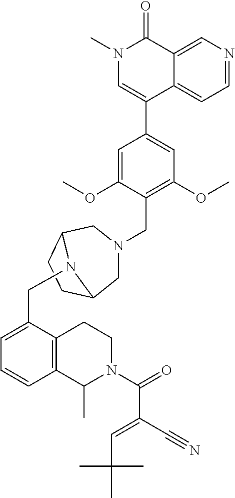 [SMILES: COc1cc(-c2cn(C)c(=O)c3cnccc23)cc(OC)c1CN1CC2CCC(C1)N2Cc1cccc2c1CCN(C(=O)/C(C#N)=C/C(C)(C)C)C2C] | A48 |
| 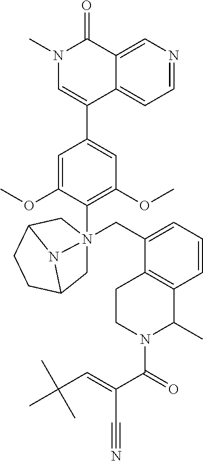 | A49 |
| 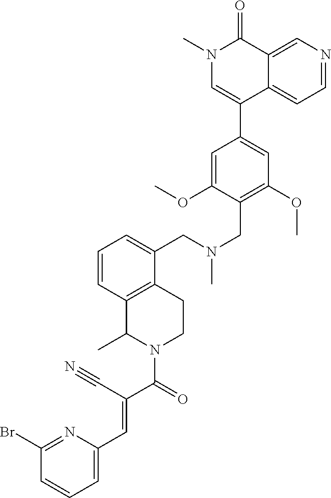 [SMILES: COc1cc(-c2cn(C)c(=O)c3cnccc23)cc(OC)c1CN(C)Cc1cccc2c1CCN(C(=O)/C(C#N)=C/c1cccc(Br)n1)C2C] | A50 |
| 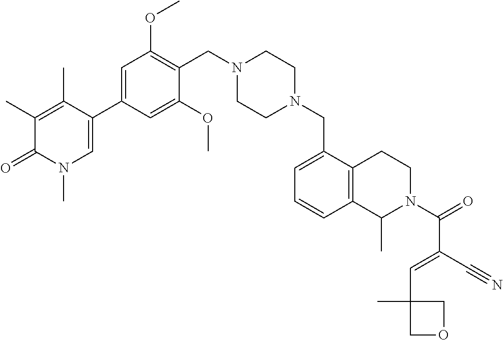 [SMILES: COc1cc(-c2cn(C)c(=O)c(C)c2C)cc(OC)c1CN1CCN(Cc2cccc3c2CCN(C(=O)/C(C#N)=C/C2(C)COC2)C3C)CC1] | A51 |
| 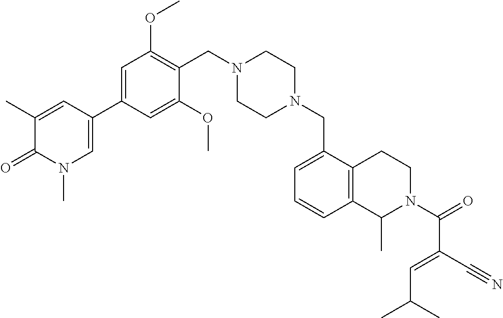 [SMILES: COc1cc(-c2cc(C)c(=O)n(C)c2)cc(OC)c1CN1CCN(Cc2cccc3c2CCN(C(=O)/C(C#N)=C/C(C)C)C3C)CC1] | A52 |
| 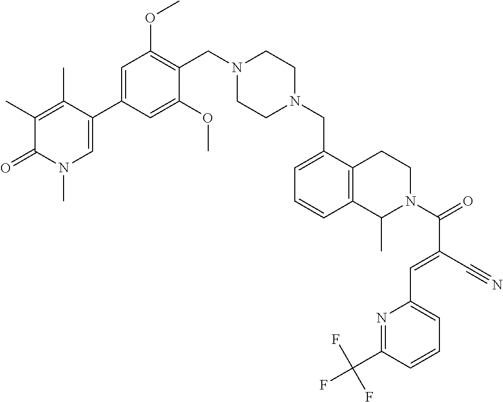 [SMILES: COc1cc(-c2cn(C)c(=O)c(C)c2C)cc(OC)c1CN1CCN(Cc2cccc3c2CCN(C(=O)/C(C#N)=C/c2cccc(C(F)(F)F)n2)C3C)CC1] | A53 |
| 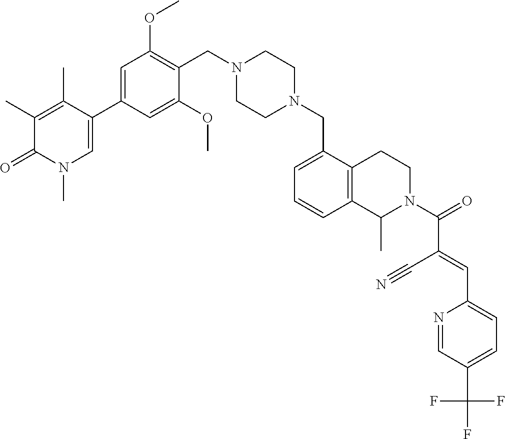 [SMILES: COc1cc(-c2cn(C)c(=O)c(C)c2C)cc(OC)c1CN1CCN(Cc2cccc3c2CCN(C(=O)/C(C#N)=C/c2ccc(C(F)(F)F)cn2)C3C)CC1] | A54 |
| 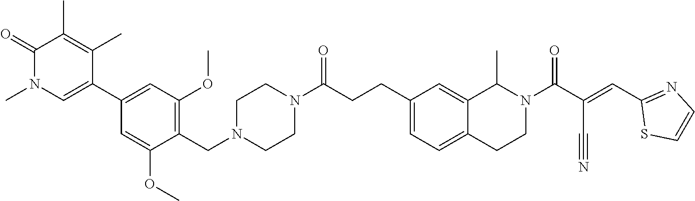 [SMILES: COc1cc(-c2cn(C)c(=O)c(C)c2C)cc(OC)c1CN1CCN(C(=O)CCc2ccc3c(c2)C(C)N(C(=O)/C(C#N)=C/c2nccs2)CC3)CC1] | A55 |
| 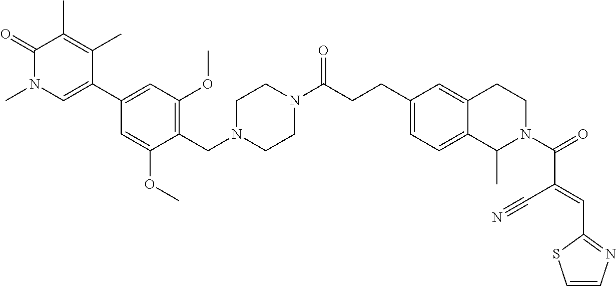 [SMILES: COc1cc(-c2cn(C)c(=O)c(C)c2C)cc(OC)c1CN1CCN(C(=O)CCc2ccc3c(c2)CCN(C(=O)/C(C#N)=C/c2nccs2)C3C)CC1] | A56 |
| 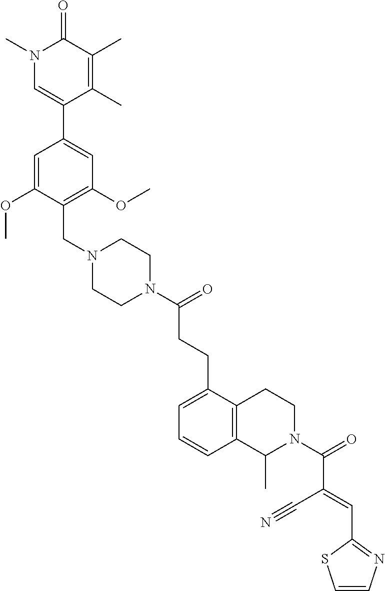 [SMILES: COc1cc(-c2cn(C)c(=O)c(C)c2C)cc(OC)c1CN1CCN(C(=O)CCc2cccc3c2CCN(C(=O)/C(C#N)=C/c2nccs2)C3C)CC1] | A57 |
| 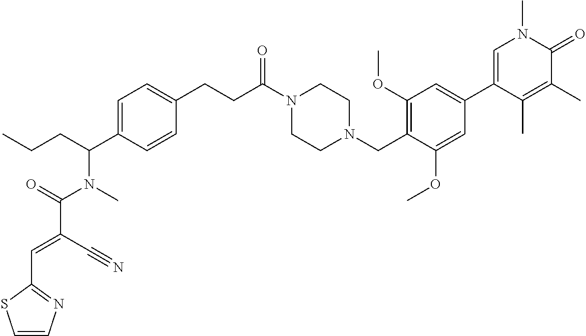 [SMILES: CCCC(c1ccc(CCC(=O)N2CCN(Cc3c(OC)cc(-c4cn(C)c(=O)c(C)c4C)cc3OC)CC2)cc1)N(C)C(=O)/C(C#N)=C/c1nccs1] | A58 |
| 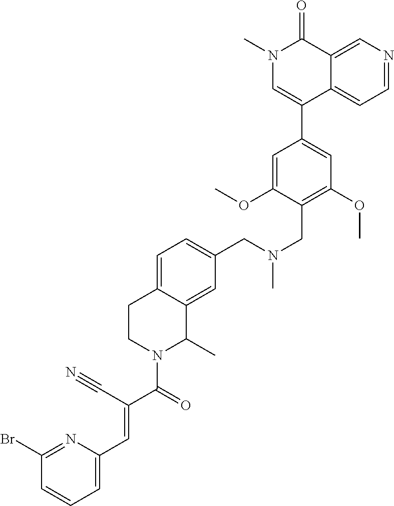 [SMILES: COc1cc(-c2cn(C)c(=O)c3cnccc23)cc(OC)c1CN(C)Cc1ccc2c(c1)C(C)N(C(=O)/C(C#N)=C/c1cccc(Br)n1)CC2] | A59 |
| 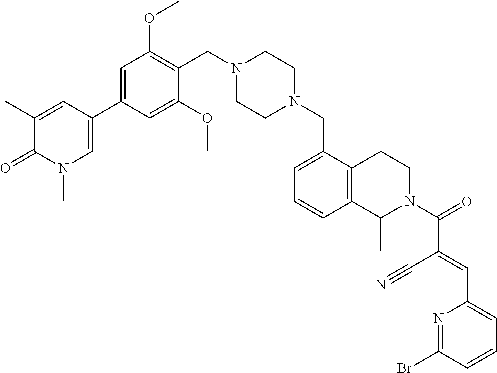 [SMILES: COc1cc(-c2cc(C)c(=O)n(C)c2)cc(OC)c1CN1CCN(Cc2cccc3c2CCN(C(=O)/C(C#N)=C/c2cccc(Br)n2)C3C)CC1] | A60 |
| 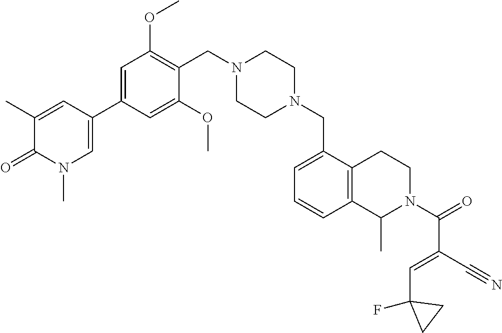 [SMILES: COc1cc(-c2cc(C)c(=O)n(C)c2)cc(OC)c1CN1CCN(Cc2cccc3c2CCN(C(=O)/C(C#N)=C/C2(F)CC2)C3C)CC1] | A61 |
| 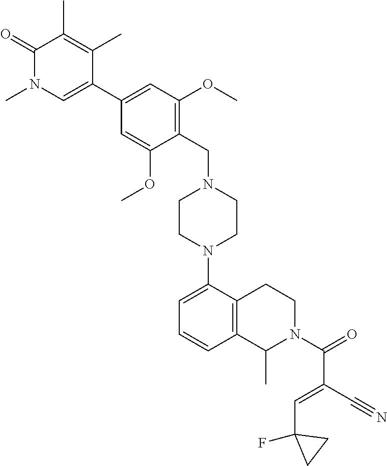 [SMILES: COc1cc(-c2cn(C)c(=O)c(C)c2C)cc(OC)c1CN1CCN(c2cccc3c2CCN(C(=O)/C(C#N)=C/C2(F)CC2)C3C)CC1] | A62 |
| 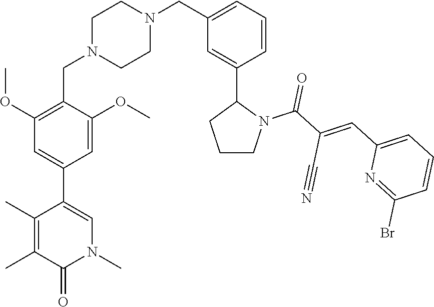 [SMILES: COc1cc(-c2cn(C)c(=O)c(C)c2C)cc(OC)c1CN1CCN(Cc2cccc(C3CCCN3C(=O)/C(C#N)=C/c3cccc(Br)n3)c2)CC1] | A63 |
| 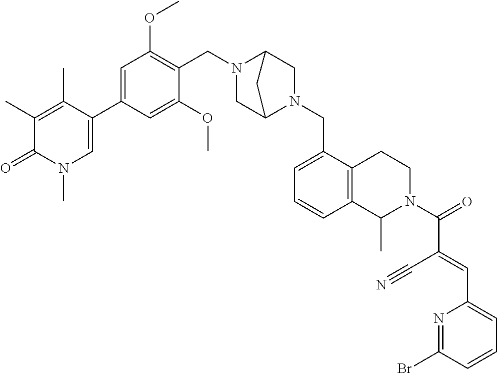 [SMILES: COc1cc(-c2cn(C)c(=O)c(C)c2C)cc(OC)c1CN1CC2CC1CN2Cc1cccc2c1CCN(C(=O)/C(C#N)=C/c1cccc(Br)n1)C2C] | A64 |
| 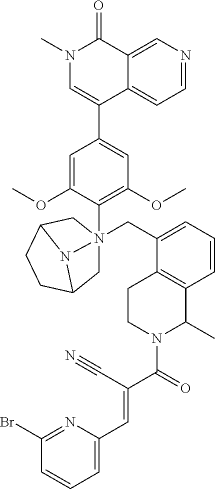 | A65 |
| 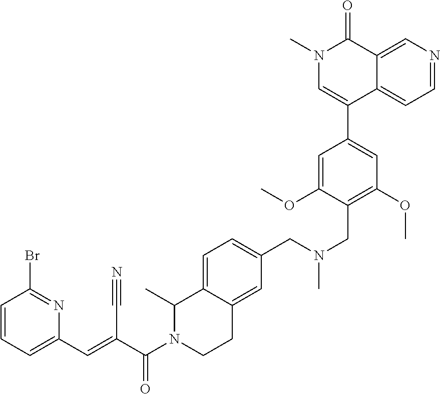 [SMILES: COc1cc(-c2cn(C)c(=O)c3cnccc23)cc(OC)c1CN(C)Cc1ccc2c(c1)CCN(C(=O)/C(C#N)=C/c1cccc(Br)n1)C2C] | A66 |
| 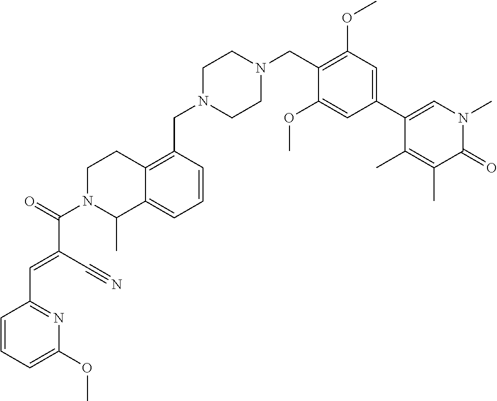 [SMILES: COc1cccc(/C=C(\C#N)C(=O)N2CCc3c(CN4CCN(Cc5c(OC)cc(-c6cn(C)c(=O)c(C)c6C)cc5OC)CC4)cccc3C2C)n1] | A67 |
| 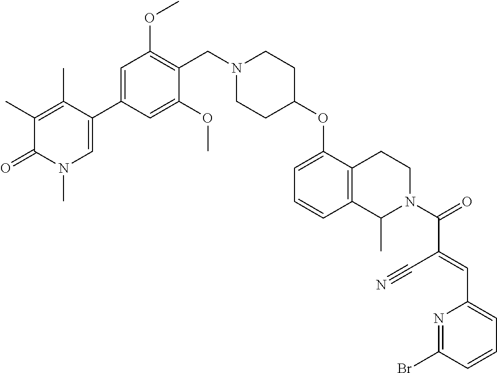 [SMILES: COc1cc(-c2cn(C)c(=O)c(C)c2C)cc(OC)c1CN1CCC(Oc2cccc3c2CCN(C(=O)/C(C#N)=C/c2cccc(Br)n2)C3C)CC1] | A68 |
| 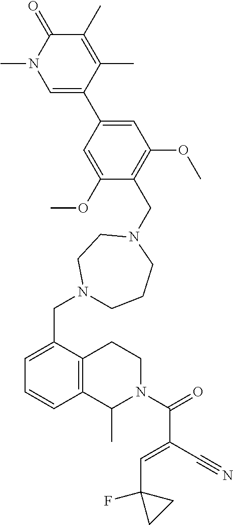 [SMILES: COc1cc(-c2cn(C)c(=O)c(C)c2C)cc(OC)c1CN1CCCN(Cc2cccc3c2CCN(C(=O)/C(C#N)=C/C2(F)CC2)C3C)CC1] | A69 |
| 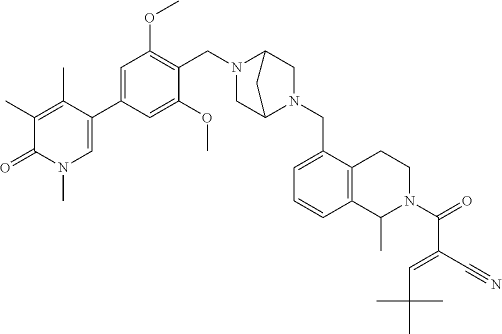 [SMILES: COc1cc(-c2cn(C)c(=O)c(C)c2C)cc(OC)c1CN1CC2CC1CN2Cc1cccc2c1CCN(C(=O)/C(C#N)=C/C(C)(C)C)C2C] | A70 |
| 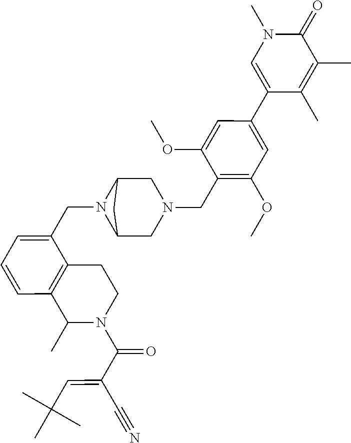 [SMILES: COc1cc(-c2cn(C)c(=O)c(C)c2C)cc(OC)c1CN1CC2CC(C1)N2Cc1cccc2c1CCN(C(=O)/C(C#N)=C/C(C)(C)C)C2C] | A71 |
| 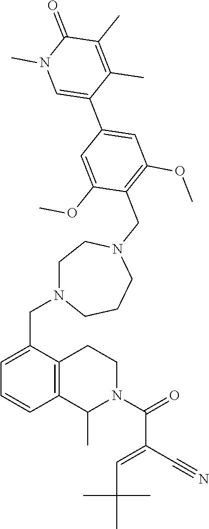 [SMILES: COc1cc(-c2cn(C)c(=O)c(C)c2C)cc(OC)c1CN1CCCN(Cc2cccc3c2CCN(C(=O)/C(C#N)=C/C(C)(C)C)C3C)CC1] | A72 |
| 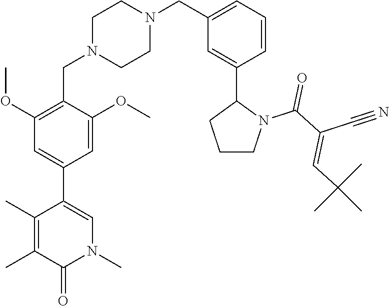 [SMILES: COc1cc(-c2cn(C)c(=O)c(C)c2C)cc(OC)c1CN1CCN(Cc2cccc(C3CCCN3C(=O)/C(C#N)=C/C(C)(C)C)c2)CC1] | A73 |
| 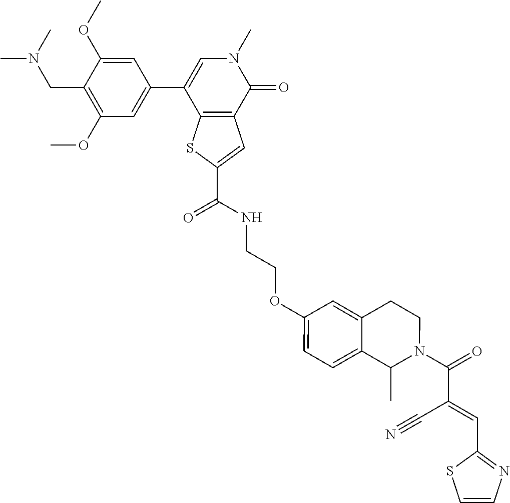 [SMILES: COc1cc(-c2cn(C)c(=O)c3cc(C(=O)NCCOc4ccc5c(c4)CCN(C(=O)/C(C#N)=C/c4nccs4)C5C)sc23)cc(OC)c1CN(C)C] | A74 |
| 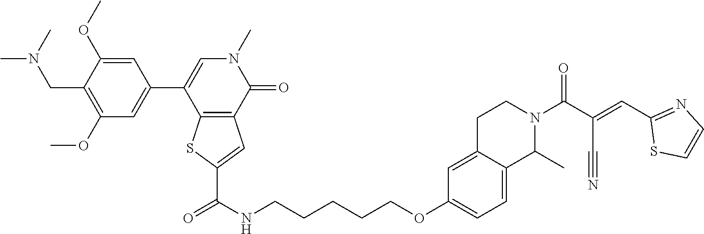 [SMILES: COc1cc(-c2cn(C)c(=O)c3cc(C(=O)NCCCCCOc4ccc5c(c4)CCN(C(=O)/C(C#N)=C/c4nccs4)C5C)sc23)cc(OC)c1CN(C)C] | A75 |
| 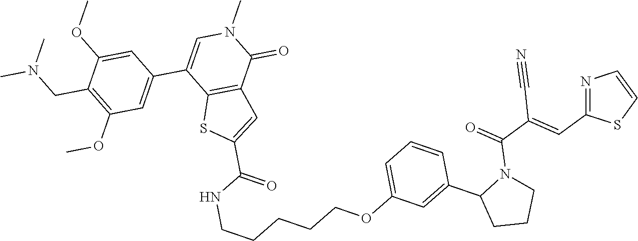 [SMILES: COc1cc(-c2cn(C)c(=O)c3cc(C(=O)NCCCCCOc4cccc(C5CCCN5C(=O)/C(C#N)=C/c5nccs5)c4)sc23)cc(OC)c1CN(C)C] | A76 |
| 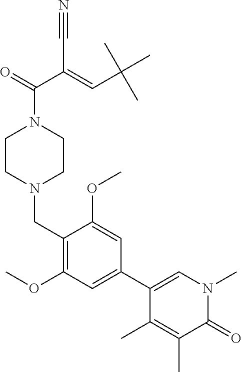 [SMILES: COc1cc(-c2cn(C)c(=O)c(C)c2C)cc(OC)c1CN1CCN(C(=O)/C(C#N)=C/C(C)(C)C)CC1] | BRD9a |
| 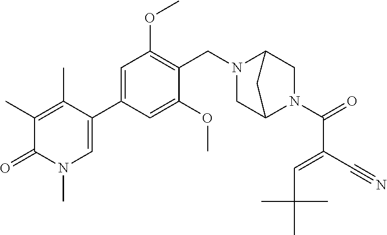 [SMILES: COc1cc(-c2cn(C)c(=O)c(C)c2C)cc(OC)c1CN1CC2CC1CN2C(=O)/C(C#N)=C/C(C)(C)C] | BRD9b |
| 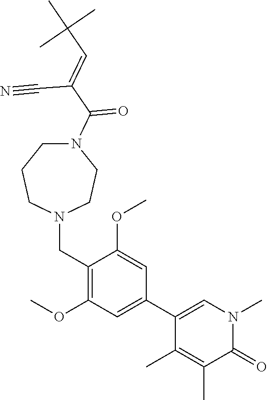 [SMILES: COc1cc(-c2cn(C)c(=O)c(C)c2C)cc(OC)c1CN1CCCN(C(=O)/C(C#N)=C/C(C)(C)C)CC1] | BRD9c |
| 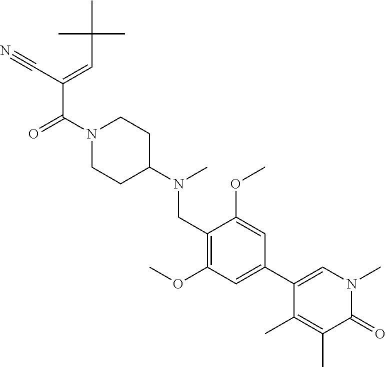 [SMILES: COc1cc(-c2cn(C)c(=O)c(C)c2C)cc(OC)c1CN(C)C1CCN(C(=O)/C(C#N)=C/C(C)(C)C)CC1] | BRD9d |
| 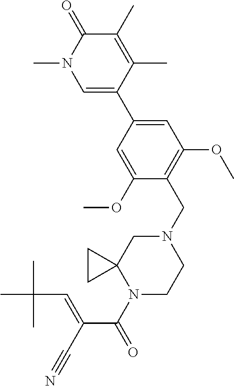 [SMILES: COc1cc(-c2cn(C)c(=O)c(C)c2C)cc(OC)c1CN1CCN(C(=O)/C(C#N)=C/C(C)(C)C)C2(CC2)C1] | BRD9e |
| 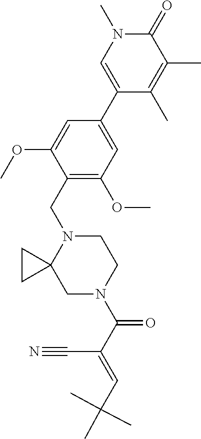 | BRD9f |
| 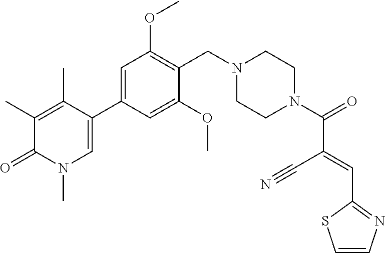 [SMILES: COc1cc(-c2cn(C)c(=O)c(C)c2C)cc(OC)c1CN1CCN(C(=O)/C(C#N)=C/c2nccs2)CC1] | BRD9g |
| 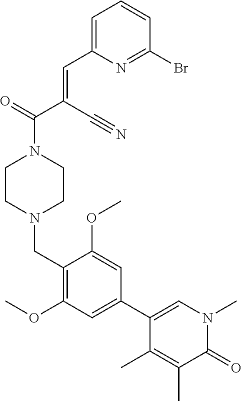 [SMILES: COc1cc(-c2cn(C)c(=O)c(C)c2C)cc(OC)c1CN1CCN(C(=O)/C(C#N)=C/c2cccc(Br)n2)CC1] | BRD9h |
| 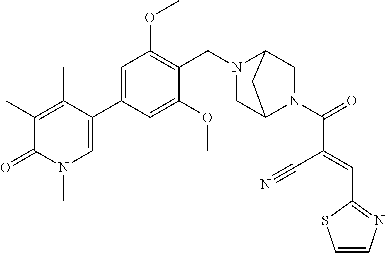 [SMILES: COc1cc(-c2cn(C)c(=O)c(C)c2C)cc(OC)c1CN1CC2CC1CN2C(=O)/C(C#N)=C/c1nccs1] | BRD9i |
| 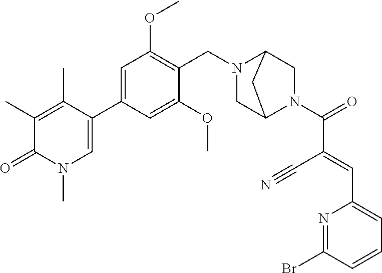 [SMILES: COc1cc(-c2cn(C)c(=O)c(C)c2C)cc(OC)c1CN1CC2CC1CN2C(=O)/C(C#N)=C/c1cccc(Br)n1] | BRD9j |
| 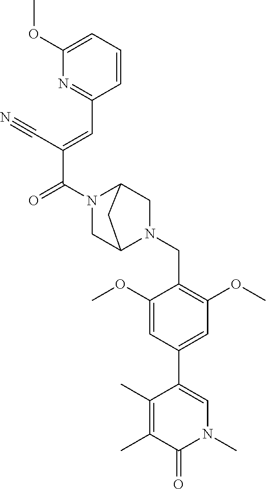 [SMILES: COc1cccc(/C=C(\C#N)C(=O)N2CC3CC2CN3Cc2c(OC)cc(-c3cn(C)c(=O)c(C)c3C)cc2OC)n1] | BRD9k |
| 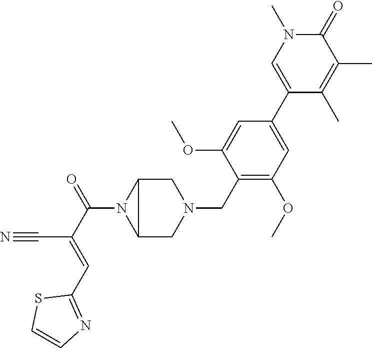 [SMILES: COc1cc(-c2cn(C)c(=O)c(C)c2C)cc(OC)c1CN1CC2C(C1)N2C(=O)/C(C#N)=C/c1nccs1] | BRD9l |
| 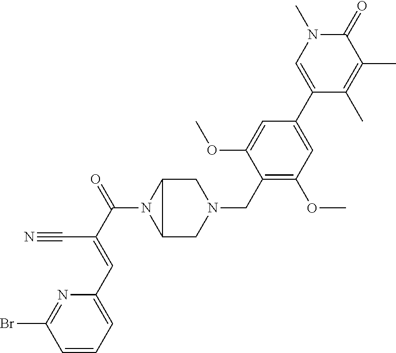 [SMILES: COc1cc(-c2cn(C)c(=O)c(C)c2C)cc(OC)c1CN1CC2C(C1)N2C(=O)/C(C#N)=C/c1cccc(Br)n1] | BRD9m |
| 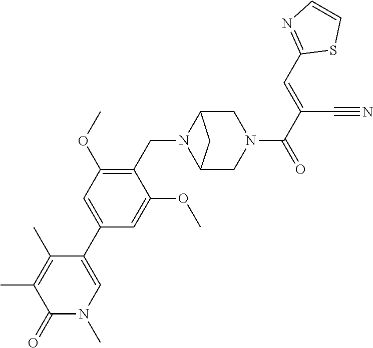 [SMILES: COc1cc(-c2cn(C)c(=O)c(C)c2C)cc(OC)c1CN1C2CC1CN(C(=O)/C(C#N)=C/c1nccs1)C2] | BRD9n |
| 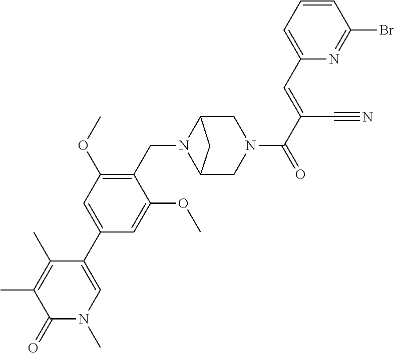 [SMILES: COc1cc(-c2cn(C)c(=O)c(C)c2C)cc(OC)c1CN1C2CC1CN(C(=O)/C(C#N)=C/c1cccc(Br)n1)C2] | BRD9o |
| 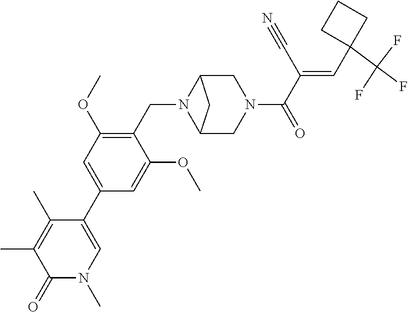 [SMILES: COc1cc(-c2cn(C)c(=O)c(C)c2C)cc(OC)c1CN1C2CC1CN(C(=O)/C(C#N)=C/C1(C(F)(F)F)CCC1)C2] | BRD9p |
| 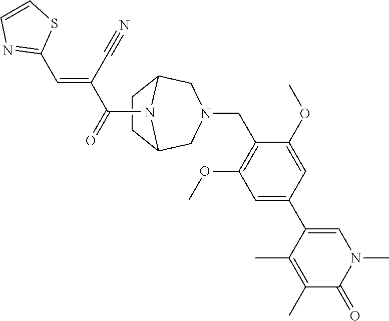 | BRD9q |
| 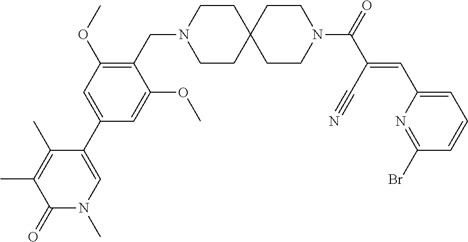 [SMILES: COc1cc(-c2cn(C)c(=O)c(C)c2C)cc(OC)c1CN1CCC2(CC1)CCN(C(=O)/C(C#N)=C/c1cccc(Br)n1)CC2] | BRD9r |
| 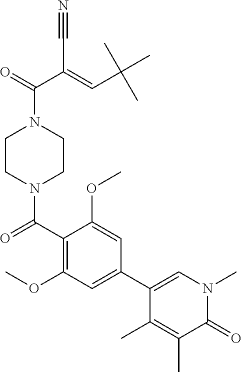 [SMILES: COc1cc(-c2cn(C)c(=O)c(C)c2C)cc(OC)c1C(=O)N1CCN(C(=O)/C(C#N)=C/C(C)(C)C)CC1] | BRD9s |
| 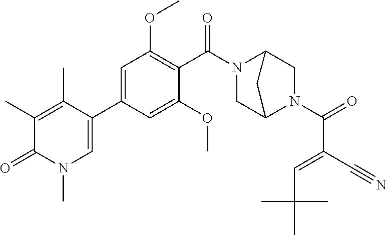 [SMILES: COc1cc(-c2cn(C)c(=O)c(C)c2C)cc(OC)c1C(=O)N1CC2CC1CN2C(=O)/C(C#N)=C/C(C)(C)C] | BRD9t |
| 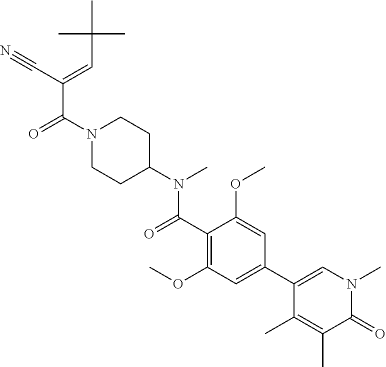 [SMILES: COc1cc(-c2cn(C)c(=O)c(C)c2C)cc(OC)c1C(=O)N(C)C1CCN(C(=O)/C(C#N)=C/C(C)(C)C)CC1] | BRD9u |
| 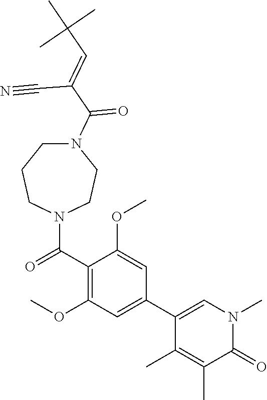 [SMILES: COc1cc(-c2cn(C)c(=O)c(C)c2C)cc(OC)c1C(=O)N1CCCN(C(=O)/C(C#N)=C/C(C)(C)C)CC1] | BRD9v |
| 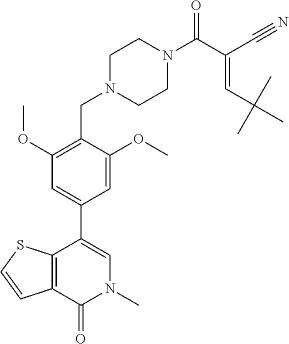 [SMILES: COc1cc(-c2cn(C)c(=O)c3ccsc23)cc(OC)c1CN1CCN(C(=O)/C(C#N)=C/C(C)(C)C)CC1] | BRD9w |
| 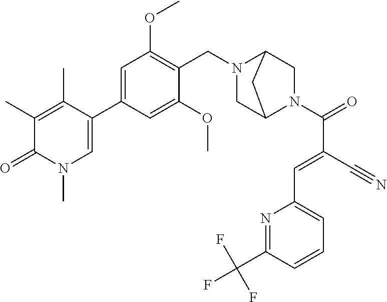 [SMILES: COc1cc(-c2cn(C)c(=O)c(C)c2C)cc(OC)c1CN1CC2CC1CN2C(=O)/C(C#N)=C/c1cccc(C(F)(F)F)n1] | BRD9x |
| 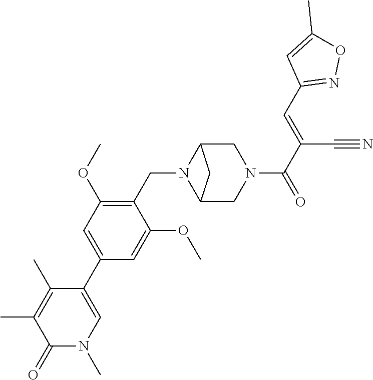 [SMILES: COc1cc(-c2cn(C)c(=O)c(C)c2C)cc(OC)c1CN1C2CC1CN(C(=O)/C(C#N)=C/c1cc(C)on1)C2] | BRD9y |
| 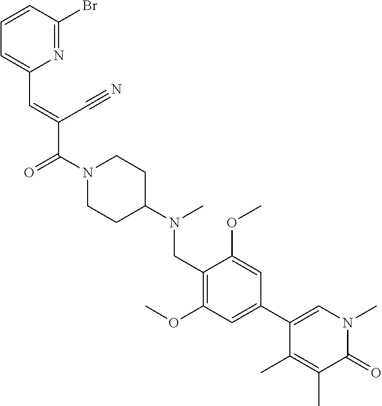 [SMILES: COc1cc(-c2cn(C)c(=O)c(C)c2C)cc(OC)c1CN(C)C1CCN(C(=O)/C(C#N)=C/c2cccc(Br)n2)CC1] | BRD9z |
| 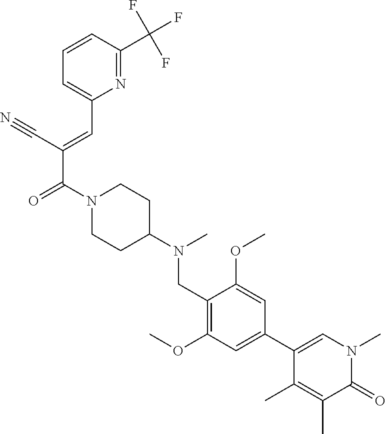 [SMILES: COc1cc(-c2cn(C)c(=O)c(C)c2C)cc(OC)c1CN(C)C1CCN(C(=O)/C(C#N)=C/c2cccc(C(F)(F)F)n2)CC1] | BRD9aa |
| 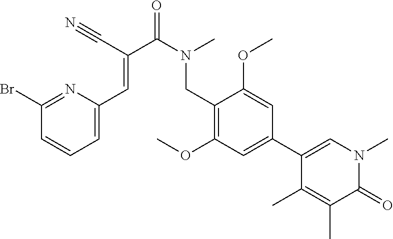 [SMILES: COc1cc(-c2cn(C)c(=O)c(C)c2C)cc(OC)c1CN(C)C(=O)/C(C#N)=C/c1cccc(Br)n1] | BRD9ab |
| 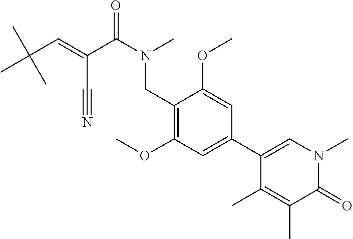 [SMILES: COc1cc(-c2cn(C)c(=O)c(C)c2C)cc(OC)c1CN(C)C(=O)/C(C#N)=C/C(C)(C)C] | BRD9ac |
| 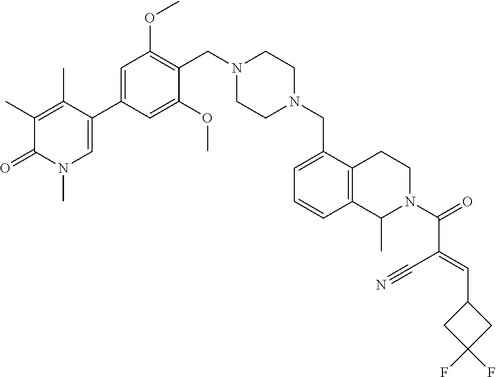 [SMILES: COc1cc(-c2cn(C)c(=O)c(C)c2C)cc(OC)c1CN1CCN(Cc2cccc3c2CCN(C(=O)/C(C#N)=C/C2CC(F)(F)C2)C3C)CC1] | B1 |
| 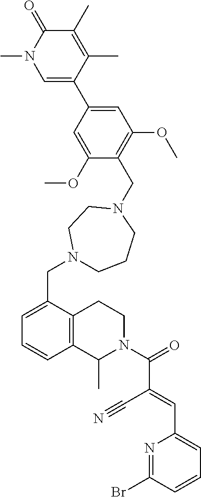 [SMILES: COc1cc(-c2cn(C)c(=O)c(C)c2C)cc(OC)c1CN1CCCN(Cc2cccc3c2CCN(C(=O)/C(C#N)=C/c2cccc(Br)n2)C3C)CC1] | B2 |
| 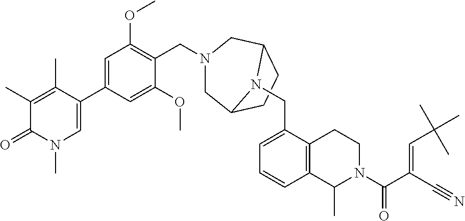 [SMILES: COc1cc(-c2cn(C)c(=O)c(C)c2C)cc(OC)c1CN1CC2CCC(C1)N2Cc1cccc2c1CCN(C(=O)/C(C#N)=C/C(C)(C)C)C2C] | B3 |
| 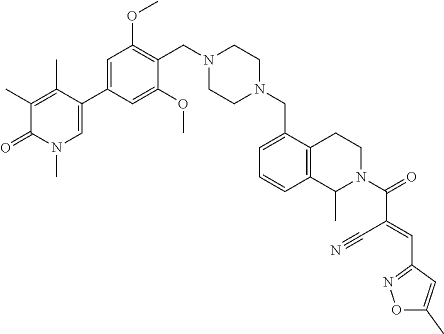 | B4 |
| 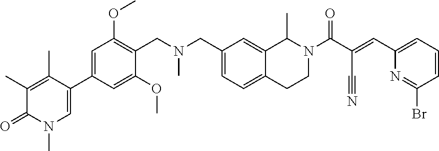 [SMILES: COc1cc(-c2cn(C)c(=O)c(C)c2C)cc(OC)c1CN(C)Cc1ccc2c(c1)C(C)N(C(=O)/C(C#N)=C/c1cccc(Br)n1)CC2] | B5 |
| 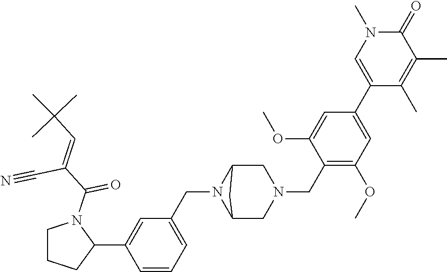 [SMILES: COc1cc(-c2cn(C)c(=O)c(C)c2C)cc(OC)c1CN1CC2CC(C1)N2Cc1cccc(C2CCCN2C(=O)/C(C#N)=C/C(C)(C)C)c1] | B6 |
| 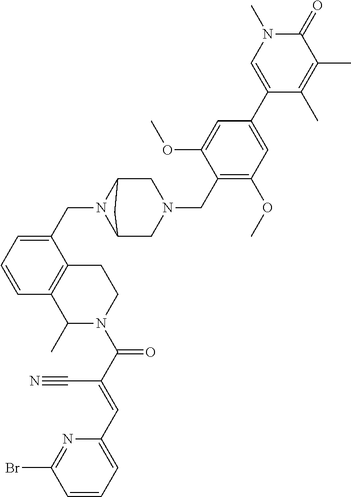 [SMILES: COc1cc(-c2cn(C)c(=O)c(C)c2C)cc(OC)c1CN1CC2CC(C1)N2Cc1cccc2c1CCN(C(=O)/C(C#N)=C/c1cccc(Br)n1)C2C] | B7 |
| 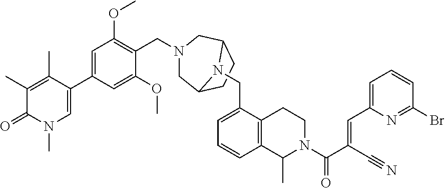 [SMILES: COc1cc(-c2cn(C)c(=O)c(C)c2C)cc(OC)c1CN1CC2CCC(C1)N2Cc1cccc2c1CCN(C(=O)/C(C#N)=C/c1cccc(Br)n1)C2C] | B8 |
| 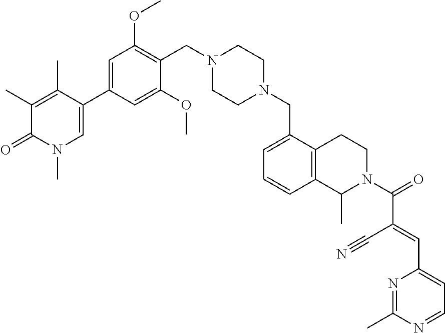 [SMILES: COc1cc(-c2cn(C)c(=O)c(C)c2C)cc(OC)c1CN1CCN(Cc2cccc3c2CCN(C(=O)/C(C#N)=C/c2ccnc(C)n2)C3C)CC1] | B9 |
| 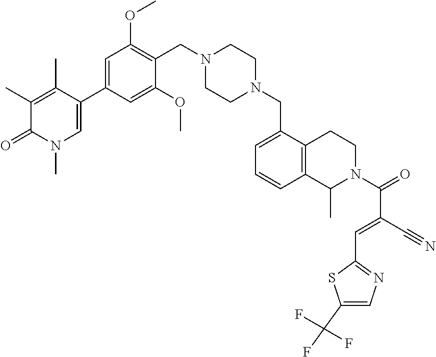 [SMILES: COc1cc(-c2cn(C)c(=O)c(C)c2C)cc(OC)c1CN1CCN(Cc2cccc3c2CCN(C(=O)/C(C#N)=C/c2ncc(C(F)(F)F)s2)C3C)CC1] | B10 |
| 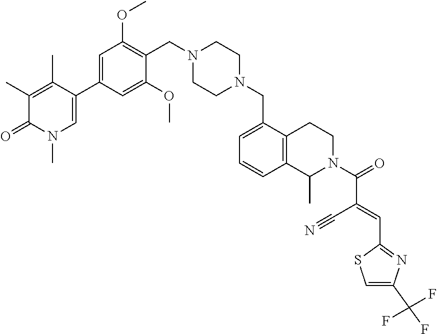 [SMILES: COc1cc(-c2cn(C)c(=O)c(C)c2C)cc(OC)c1CN1CCN(Cc2cccc3c2CCN(C(=O)/C(C#N)=C/c2nc(C(F)(F)F)cs2)C3C)CC1] | B11 |
| 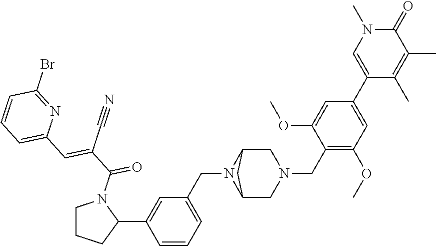 [SMILES: COc1cc(-c2cn(C)c(=O)c(C)c2C)cc(OC)c1CN1CC2CC(C1)N2Cc1cccc(C2CCCN2C(=O)/C(C#N)=C/c2cccc(Br)n2)c1] | B12 |
| 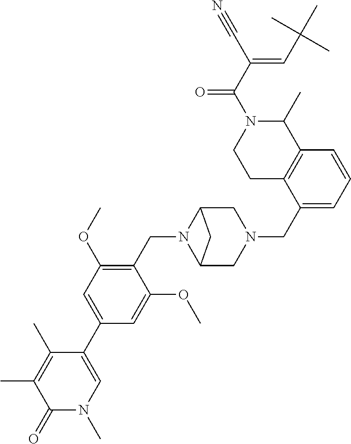 [SMILES: COc1cc(-c2cn(C)c(=O)c(C)c2C)cc(OC)c1CN1C2CC1CN(Cc1cccc3c1CCN(C(=O)/C(C#N)=C/C(C)(C)C)C3C)C2] | B13 |
| 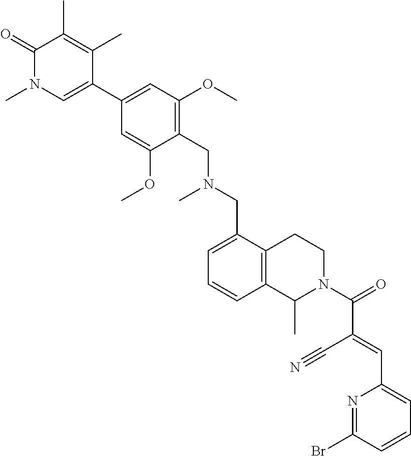 [SMILES: COc1cc(-c2cn(C)c(=O)c(C)c2C)cc(OC)c1CN(C)Cc1cccc2c1CCN(C(=O)/C(C#N)=C/c1cccc(Br)n1)C2C] | B14 |
| 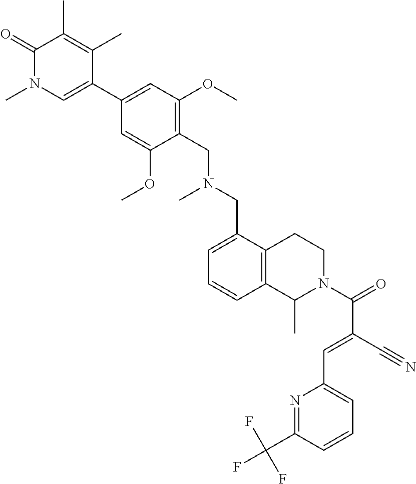 [SMILES: COc1cc(-c2cn(C)c(=O)c(C)c2C)cc(OC)c1CN(C)Cc1cccc2c1CCN(C(=O)/C(C#N)=C/c1cccc(C(F)(F)F)n1)C2C] | B15 |
| 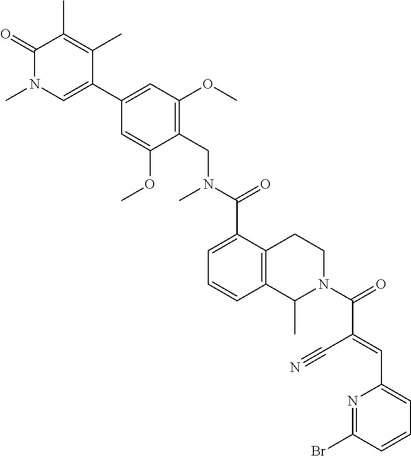 [SMILES: COc1cc(-c2cn(C)c(=O)c(C)c2C)cc(OC)c1CN(C)C(=O)c1cccc2c1CCN(C(=O)/C(C#N)=C/c1cccc(Br)n1)C2C] | B16 |
| 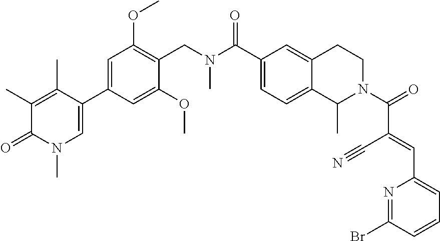 [SMILES: COc1cc(-c2cn(C)c(=O)c(C)c2C)cc(OC)c1CN(C)C(=O)c1ccc2c(c1)CCN(C(=O)/C(C#N)=C/c1cccc(Br)n1)C2C] | B17 |
| 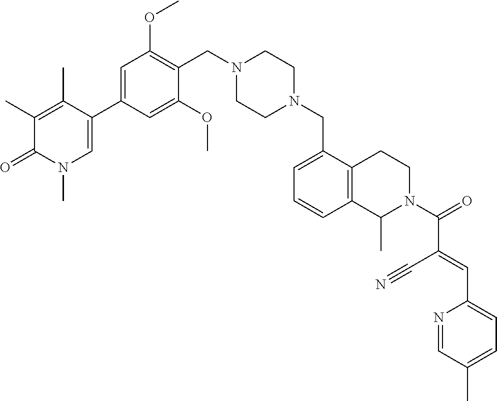 [SMILES: COc1cc(-c2cn(C)c(=O)c(C)c2C)cc(OC)c1CN1CCN(Cc2cccc3c2CCN(C(=O)/C(C#N)=C/c2ccc(C)cn2)C3C)CC1] | B18 |
| 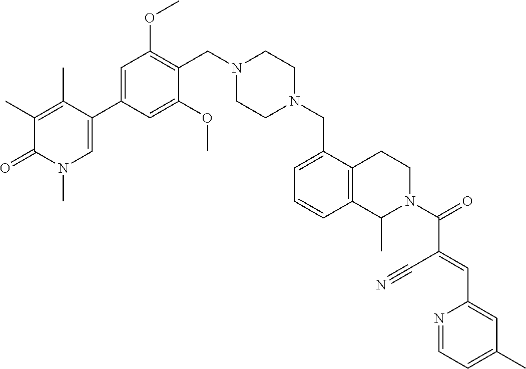 | B19 |
| 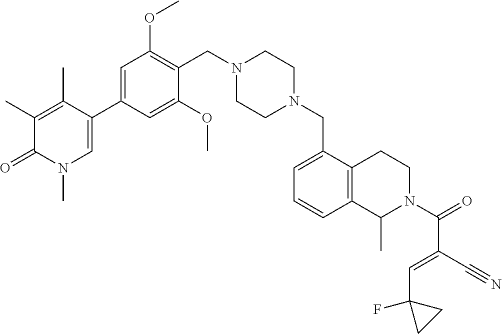 [SMILES: COc1cc(-c2cn(C)c(=O)c(C)c2C)cc(OC)c1CN1CCN(Cc2cccc3c2CCN(C(=O)/C(C#N)=C/C2(F)CC2)C3C)CC1] | B20 |
| 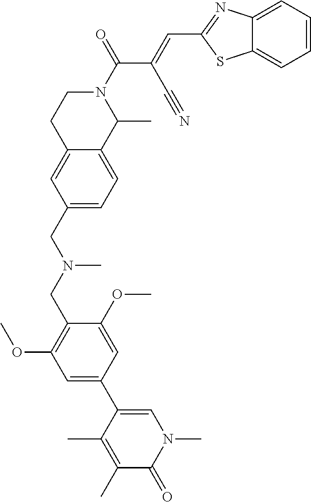 [SMILES: COc1cc(-c2cn(C)c(=O)c(C)c2C)cc(OC)c1CN(C)Cc1ccc2c(c1)CCN(C(=O)/C(C#N)=C/c1nc3ccccc3s1)C2C] | B21 |
| 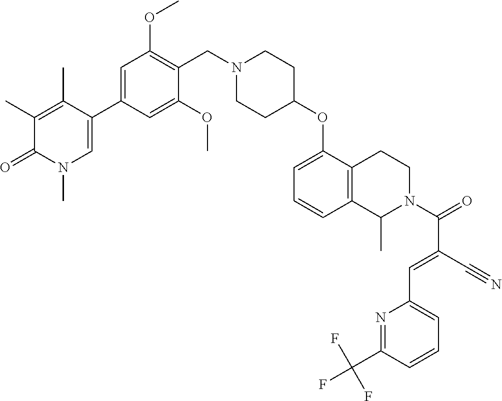 [SMILES: COc1cc(-c2cn(C)c(=O)c(C)c2C)cc(OC)c1CN1CCC(Oc2cccc3c2CCN(C(=O)/C(C#N)=C/c2cccc(C(F)(F)F)n2)C3C)CC1] | B22 |
| 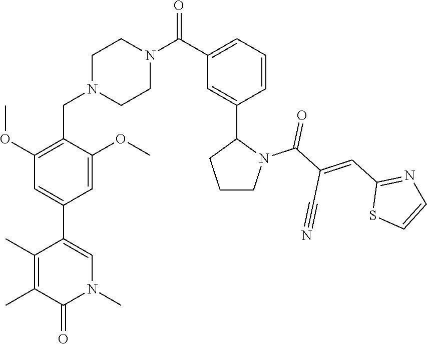 [SMILES: COc1cc(-c2cn(C)c(=O)c(C)c2C)cc(OC)c1CN1CCN(C(=O)c2cccc(C3CCCN3C(=O)/C(C#N)=C/c3nccs3)c2)CC1] | B23 |
| 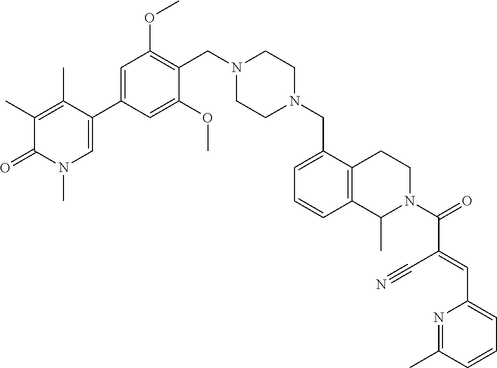 [SMILES: COc1cc(-c2cn(C)c(=O)c(C)c2C)cc(OC)c1CN1CCN(Cc2cccc3c2CCN(C(=O)/C(C#N)=C/c2cccc(C)n2)C3C)CC1] | B24 |
| 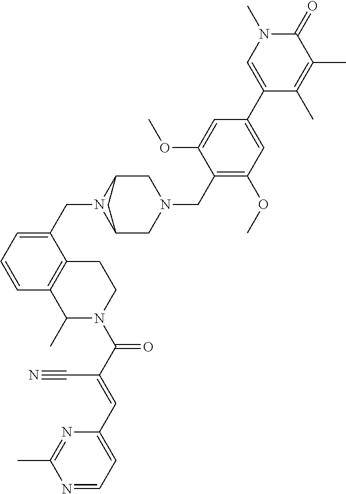 [SMILES: COc1cc(-c2cn(C)c(=O)c(C)c2C)cc(OC)c1CN1CC2CC(C1)N2Cc1cccc2c1CCN(C(=O)/C(C#N)=C/c1ccnc(C)n1)C2C] | B25 |
| 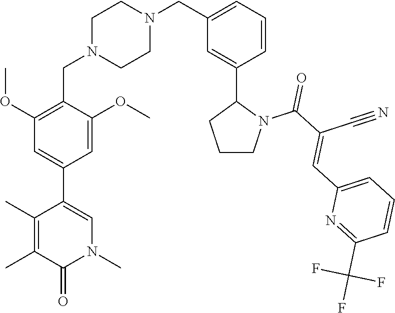 [SMILES: COc1cc(-c2cn(C)c(=O)c(C)c2C)cc(OC)c1CN1CCN(Cc2cccc(C3CCCN3C(=O)/C(C#N)=C/c3cccc(C(F)(F)F)n3)c2)CC1] | B26 |
| 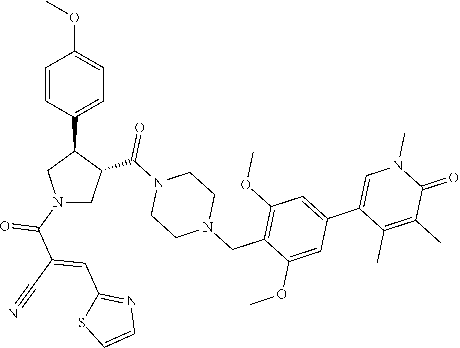 [SMILES: COc1ccc([C@@H]2CN(C(=O)/C(C#N)=C/c3nccs3)C[C@H]2C(=O)N2CCN(Cc3c(OC)cc(-c4cn(C)c(=O)c(C)c4C)cc3OC)CC2)cc1] | B27 |
| 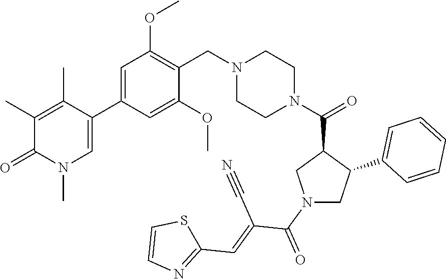 [SMILES: COc1cc(-c2cn(C)c(=O)c(C)c2C)cc(OC)c1CN1CCN(C(=O)[C@@H]2CN(C(=O)/C(C#N)=C/c3nccs3)C[C@H]2c2ccccc2)CC1] | B28 |
| 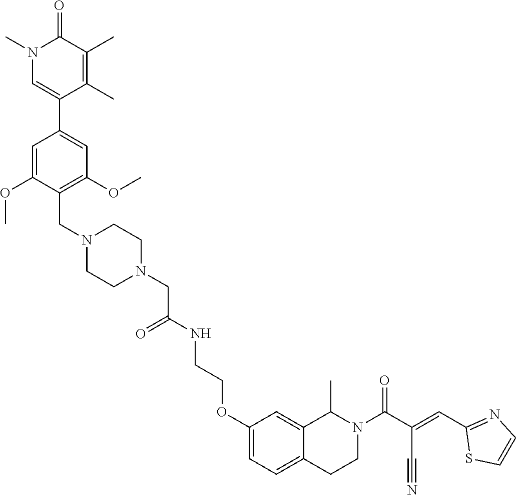 [SMILES: COc1cc(-c2cn(C)c(=O)c(C)c2C)cc(OC)c1CN1CCN(CC(=O)NCCOc2ccc3c(c2)C(C)N(C(=O)/C(C#N)=C/c2nccs2)CC3)CC1] | B29 |
| 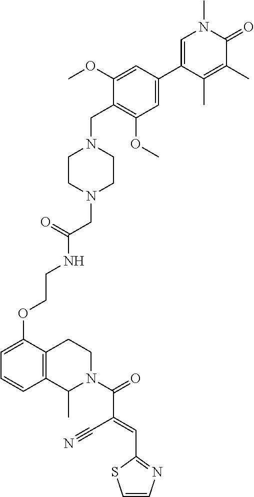 [SMILES: COc1cc(-c2cn(C)c(=O)c(C)c2C)cc(OC)c1CN1CCN(CC(=O)NCCOc2cccc3c2CCN(C(=O)/C(C#N)=C/c2nccs2)C3C)CC1] | B30 |
| 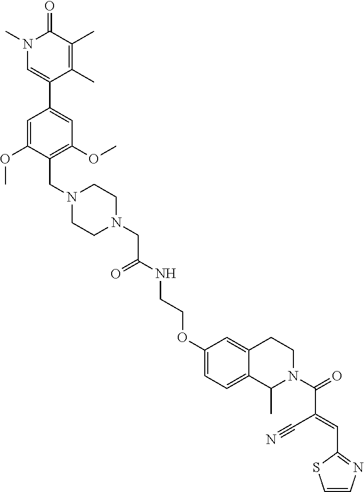 [SMILES: COc1cc(-c2cn(C)c(=O)c(C)c2C)cc(OC)c1CN1CCN(CC(=O)NCCOc2ccc3c(c2)CCN(C(=O)/C(C#N)=C/c2nccs2)C3C)CC1] | B31 |
| 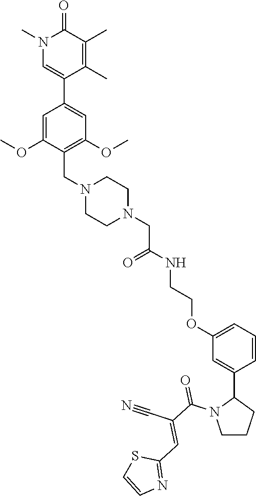 [SMILES: COc1cc(-c2cn(C)c(=O)c(C)c2C)cc(OC)c1CN1CCN(CC(=O)NCCOc2cccc(C3CCCN3C(=O)/C(C#N)=C/c3nccs3)c2)CC1] | B32 |
| 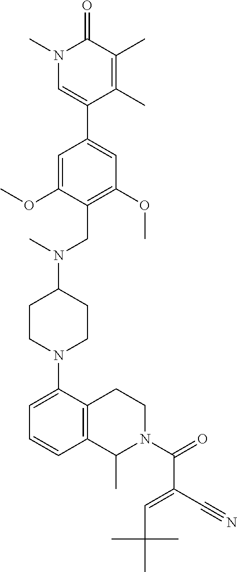 [SMILES: COc1cc(-c2cn(C)c(=O)c(C)c2C)cc(OC)c1CN(C)C1CCN(c2cccc3c2CCN(C(=O)/C(C#N)=C/C(C)(C)C)C3C)CC1] | B33 |
| 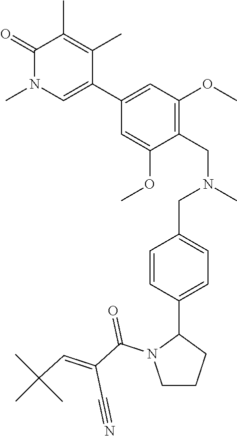 [SMILES: COc1cc(-c2cn(C)c(=O)c(C)c2C)cc(OC)c1CN(C)Cc1ccc(C2CCCN2C(=O)/C(C#N)=C/C(C)(C)C)cc1] | B34 |
| 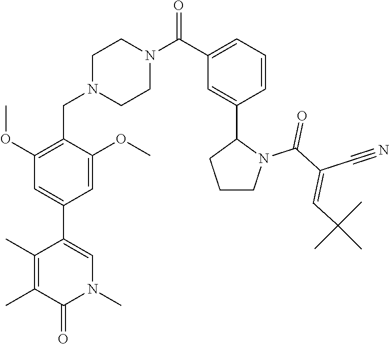 [SMILES: COc1cc(-c2cn(C)c(=O)c(C)c2C)cc(OC)c1CN1CCN(C(=O)c2cccc(C3CCCN3C(=O)/C(C#N)=C/C(C)(C)C)c2)CC1] | B35 |
| 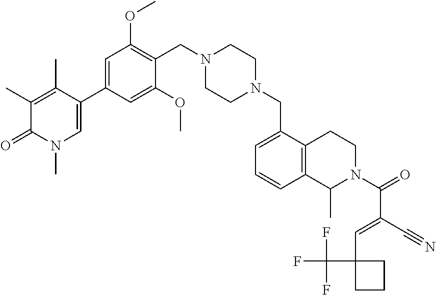 [SMILES: COc1cc(-c2cn(C)c(=O)c(C)c2C)cc(OC)c1CN1CCN(Cc2cccc3c2CCN(C(=O)/C(C#N)=C/C2(C(F)(F)F)CCC2)C3C)CC1] | B36 |
| 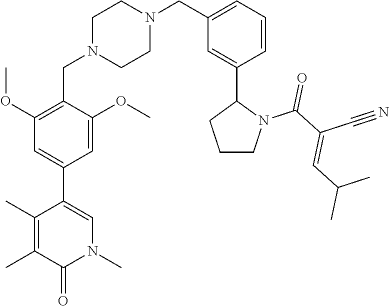 [SMILES: COc1cc(-c2cn(C)c(=O)c(C)c2C)cc(OC)c1CN1CCN(Cc2cccc(C3CCCN3C(=O)/C(C#N)=C/C(C)C)c2)CC1] | B37 |
| 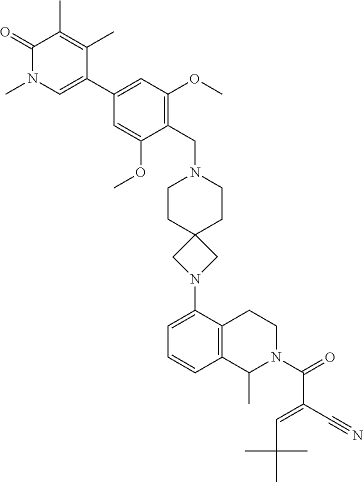 [SMILES: COc1cc(-c2cn(C)c(=O)c(C)c2C)cc(OC)c1CN1CCC2(CC1)CN(c1cccc3c1CCN(C(=O)/C(C#N)=C/C(C)(C)C)C3C)C2] | B38 |
| 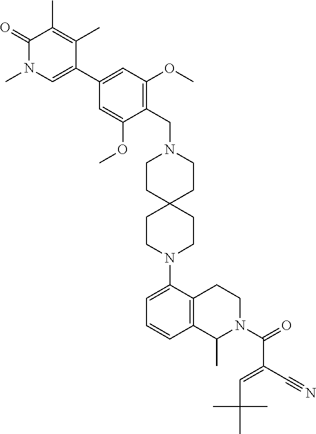 [SMILES: COc1cc(-c2cn(C)c(=O)c(C)c2C)cc(OC)c1CN1CCC2(CC1)CCN(c1cccc3c1CCN(C(=O)/C(C#N)=C/C(C)(C)C)C3C)CC2] | B39 |
| 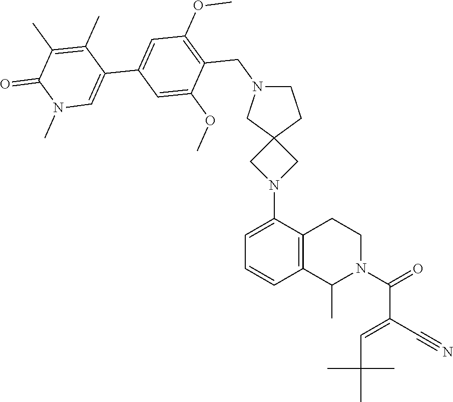 [SMILES: COc1cc(-c2cn(C)c(=O)c(C)c2C)cc(OC)c1CN1CCC2(C1)CN(c1cccc3c1CCN(C(=O)/C(C#N)=C/C(C)(C)C)C3C)C2] | B40 |
| 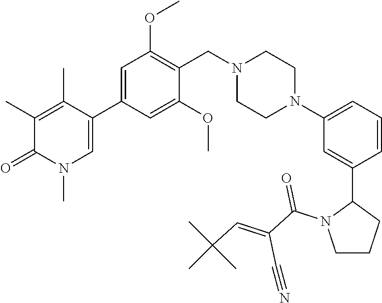 [SMILES: COc1cc(-c2cn(C)c(=O)c(C)c2C)cc(OC)c1CN1CCN(c2cccc(C3CCCN3C(=O)/C(C#N)=C/C(C)(C)C)c2)CC1] | B41 |
| 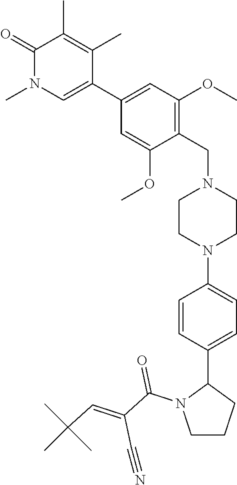 [SMILES: COc1cc(-c2cn(C)c(=O)c(C)c2C)cc(OC)c1CN1CCN(c2ccc(C3CCCN3C(=O)/C(C#N)=C/C(C)(C)C)cc2)CC1] | B42 |
| 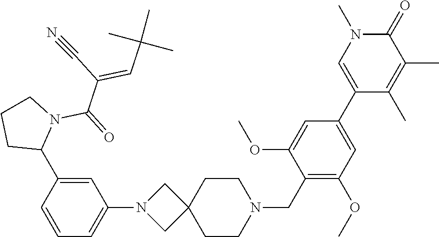 [SMILES: COc1cc(-c2cn(C)c(=O)c(C)c2C)cc(OC)c1CN1CCC2(CC1)CN(c1cccc(C3CCCN3C(=O)/C(C#N)=C/C(C)(C)C)c1)C2] | B43 |
| 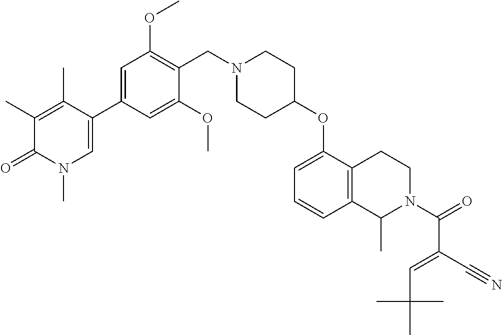 [SMILES: COc1cc(-c2cn(C)c(=O)c(C)c2C)cc(OC)c1CN1CCC(Oc2cccc3c2CCN(C(=O)/C(C#N)=C/C(C)(C)C)C3C)CC1] | B44 |
| 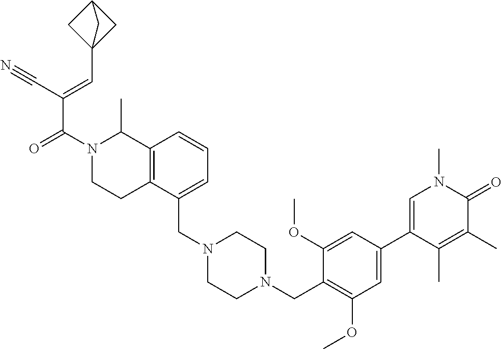 [SMILES: COc1cc(-c2cn(C)c(=O)c(C)c2C)cc(OC)c1CN1CCN(Cc2cccc3c2CCN(C(=O)/C(C#N)=C/C24CC(C2)C4)C3C)CC1] | B45 |
| 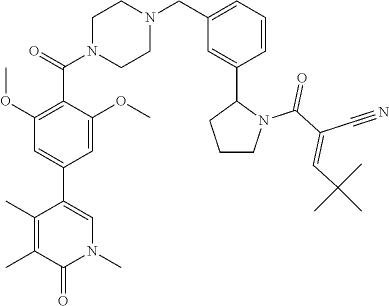 [SMILES: COc1cc(-c2cn(C)c(=O)c(C)c2C)cc(OC)c1C(=O)N1CCN(Cc2cccc(C3CCCN3C(=O)/C(C#N)=C/C(C)(C)C)c2)CC1] | B46 |
| 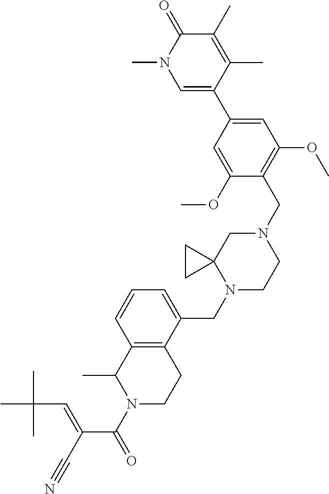 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)C2(CC2)C1] | B47 |
| 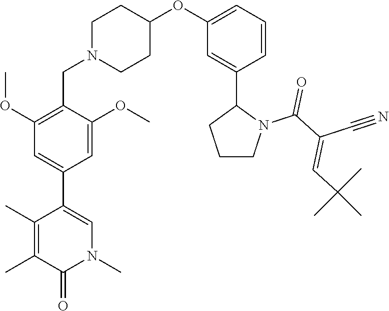 [SMILES: COc1cc(-c2cn(C)c(=O)c(C)c2C)cc(OC)c1CN1CCC(Oc2cccc(C3CCCN3C(=O)/C(C#N)=C/C(C)(C)C)c2)CC1] | B48 |
| 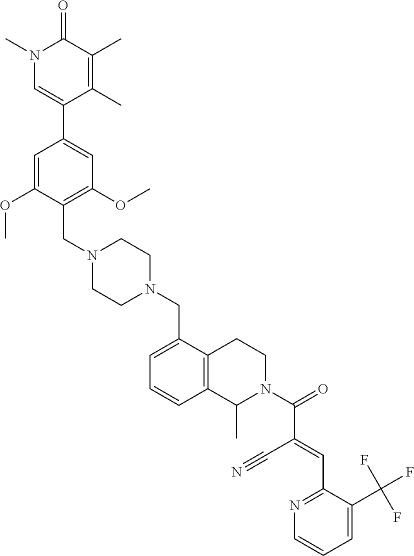 [SMILES: COc1cc(-c2cn(C)c(=O)c(C)c2C)cc(OC)c1CN1CCN(Cc2cccc3c2CCN(C(=O)/C(C#N)=C/c2ncccc2C(F)(F)F)C3C)CC1] | B49 |
| 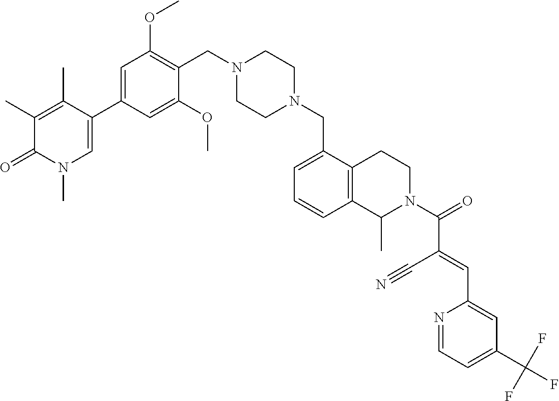 [SMILES: COc1cc(-c2cn(C)c(=O)c(C)c2C)cc(OC)c1CN1CCN(Cc2cccc3c2CCN(C(=O)/C(C#N)=C/c2cc(C(F)(F)F)ccn2)C3C)CC1] | B50 |
| 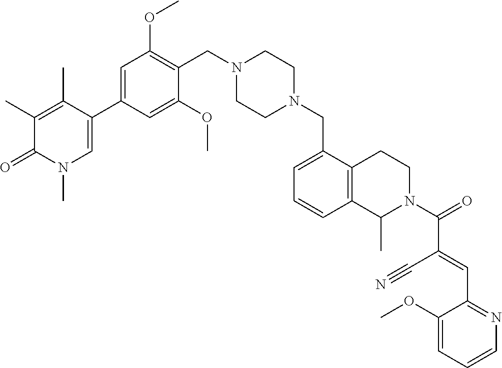 [SMILES: COc1cccnc1/C=C(\C#N)C(=O)N1CCc2c(CN3CCN(Cc4c(OC)cc(-c5cn(C)c(=O)c(C)c5C)cc4OC)CC3)cccc2C1C] | B51 |
| 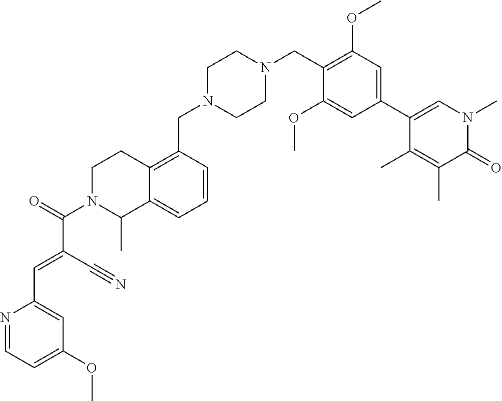 [SMILES: COc1ccnc(/C=C(\C#N)C(=O)N2CCc3c(CN4CCN(Cc5c(OC)cc(-c6cn(C)c(=O)c(C)c6C)cc5OC)CC4)cccc3C2C)c1] | B52 |
| 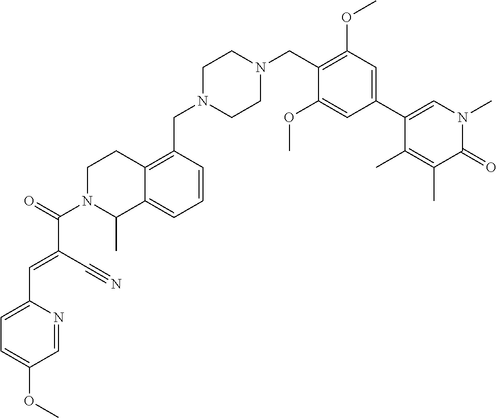 [SMILES: COc1ccc(/C=C(\C#N)C(=O)N2CCc3c(CN4CCN(Cc5c(OC)cc(-c6cn(C)c(=O)c(C)c6C)cc5OC)CC4)cccc3C2C)nc1] | B53 |
| 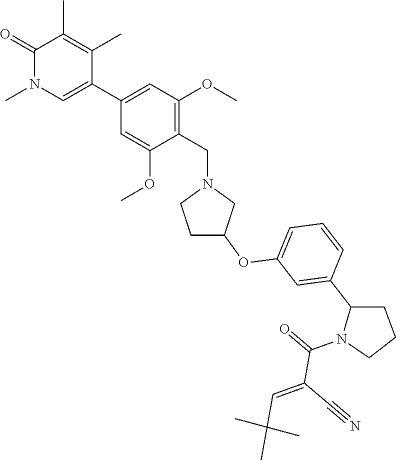 [SMILES: COc1cc(-c2cn(C)c(=O)c(C)c2C)cc(OC)c1CN1CCC(Oc2cccc(C3CCCN3C(=O)/C(C#N)=C/C(C)(C)C)c2)C1] | B54 |
| 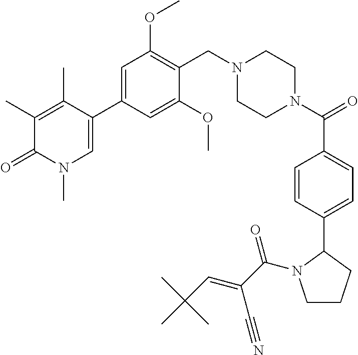 [SMILES: COc1cc(-c2cn(C)c(=O)c(C)c2C)cc(OC)c1CN1CCN(C(=O)c2ccc(C3CCCN3C(=O)/C(C#N)=C/C(C)(C)C)cc2)CC1] | B55 |
| 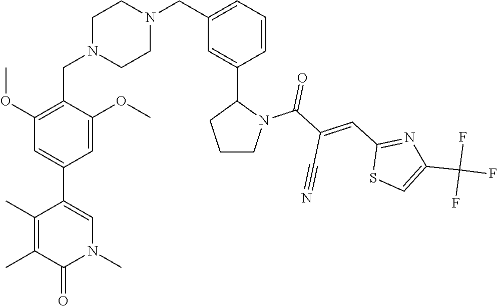 [SMILES: COc1cc(-c2cn(C)c(=O)c(C)c2C)cc(OC)c1CN1CCN(Cc2cccc(C3CCCN3C(=O)/C(C#N)=C/c3nc(C(F)(F)F)cs3)c2)CC1] | B56 |
| 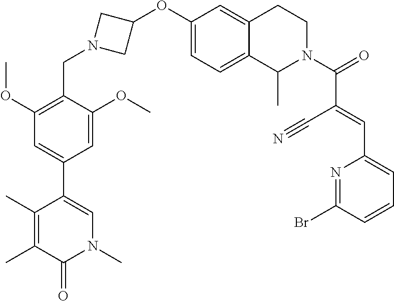 [SMILES: COc1cc(-c2cn(C)c(=O)c(C)c2C)cc(OC)c1CN1CC(Oc2ccc3c(c2)CCN(C(=O)/C(C#N)=C/c2cccc(Br)n2)C3C)C1] | B57 |
| 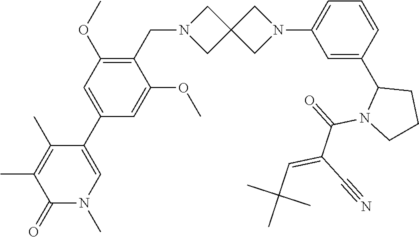 [SMILES: COc1cc(-c2cn(C)c(=O)c(C)c2C)cc(OC)c1CN1CC2(C1)CN(c1cccc(C3CCCN3C(=O)/C(C#N)=C/C(C)(C)C)c1)C2] | B58 |
| 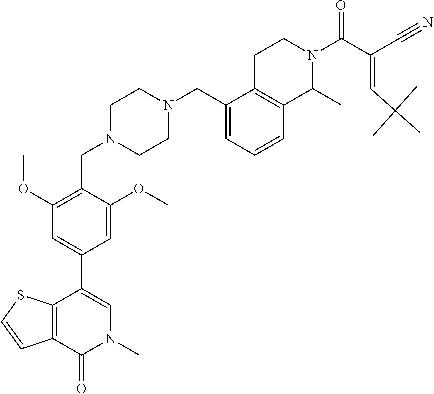 [SMILES: COc1cc(-c2cn(C)c(=O)c3ccsc23)cc(OC)c1CN1CCN(Cc2cccc3c2CCN(C(=O)/C(C#N)=C/C(C)(C)C)C3C)CC1] | B59 |
| 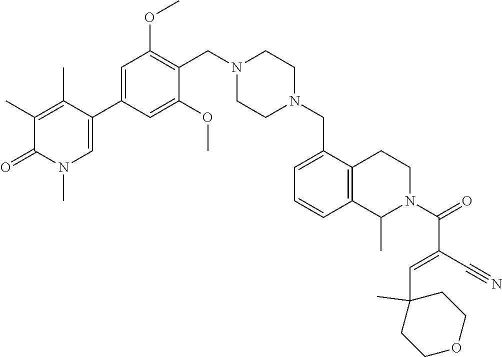 [SMILES: COc1cc(-c2cn(C)c(=O)c(C)c2C)cc(OC)c1CN1CCN(Cc2cccc3c2CCN(C(=O)/C(C#N)=C/C2(C)CCOCC2)C3C)CC1] | B60 |
| 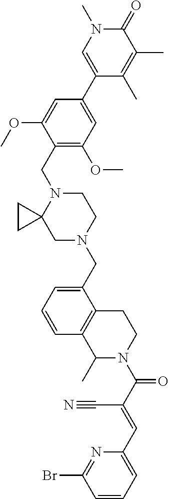 | B61 |
| 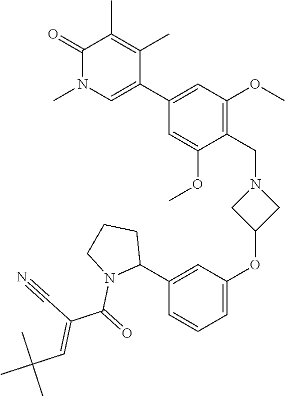 [SMILES: COc1cc(-c2cn(C)c(=O)c(C)c2C)cc(OC)c1CN1CC(Oc2cccc(C3CCCN3C(=O)/C(C#N)=C/C(C)(C)C)c2)C1] | B62 |
| 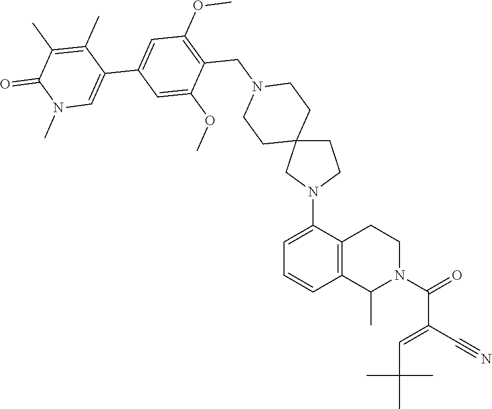 [SMILES: COc1cc(-c2cn(C)c(=O)c(C)c2C)cc(OC)c1CN1CCC2(CC1)CCN(c1cccc3c1CCN(C(=O)/C(C#N)=C/C(C)(C)C)C3C)C2] | B63 |
| 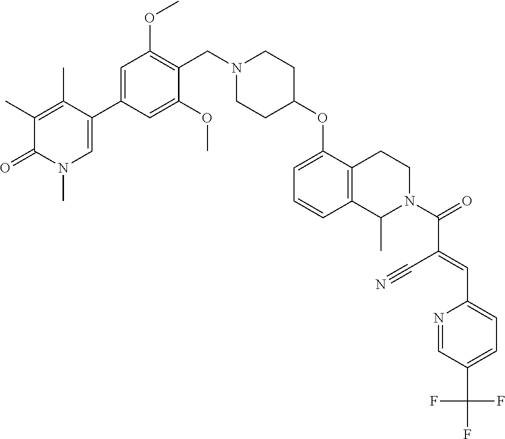 [SMILES: COc1cc(-c2cn(C)c(=O)c(C)c2C)cc(OC)c1CN1CCC(Oc2cccc3c2CCN(C(=O)/C(C#N)=C/c2ccc(C(F)(F)F)cn2)C3C)CC1] | B64 |
| 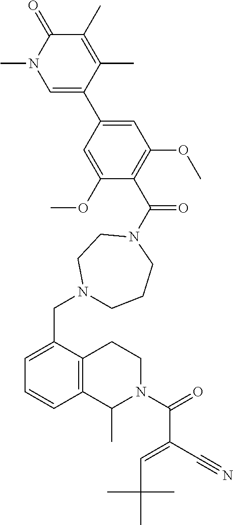 [SMILES: COc1cc(-c2cn(C)c(=O)c(C)c2C)cc(OC)c1C(=O)N1CCCN(Cc2cccc3c2CCN(C(=O)/C(C#N)=C/C(C)(C)C)C3C)CC1] | B65 |
| 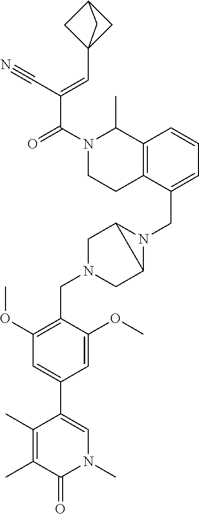 [SMILES: COc1cc(-c2cn(C)c(=O)c(C)c2C)cc(OC)c1CN1CC2C(C1)N2Cc1cccc2c1CCN(C(=O)/C(C#N)=C/C13CC(C1)C3)C2C] | B66 |
| 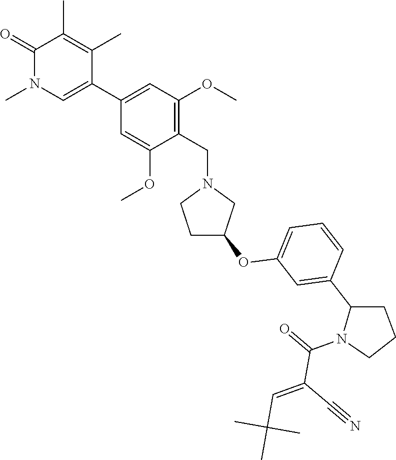 [SMILES: COc1cc(-c2cn(C)c(=O)c(C)c2C)cc(OC)c1CN1CC[C@H](Oc2cccc(C3CCCN3C(=O)/C(C#N)=C/C(C)(C)C)c2)C1] | B67 |
| 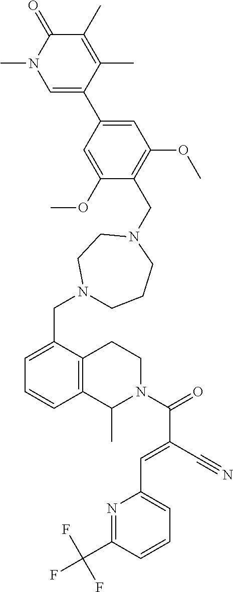 [SMILES: COc1cc(-c2cn(C)c(=O)c(C)c2C)cc(OC)c1CN1CCCN(Cc2cccc3c2CCN(C(=O)/C(C#N)=C/c2cccc(C(F)(F)F)n2)C3C)CC1] | B68 |
| 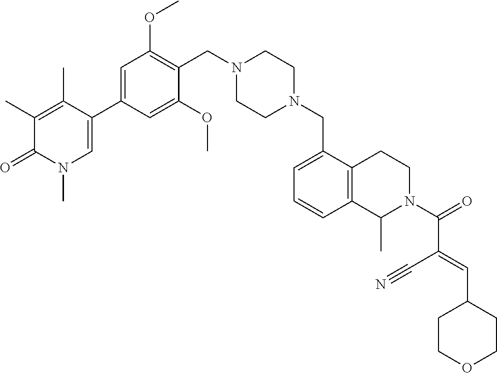 [SMILES: COc1cc(-c2cn(C)c(=O)c(C)c2C)cc(OC)c1CN1CCN(Cc2cccc3c2CCN(C(=O)/C(C#N)=C/C2CCOCC2)C3C)CC1] | B69 |
| 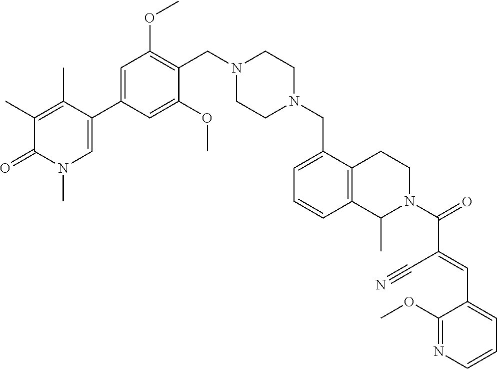 [SMILES: COc1cc(-c2cn(C)c(=O)c(C)c2C)cc(OC)c1CN1CCN(Cc2cccc3c2CCN(C(=O)/C(C#N)=C/c2cccnc2OC)C3C)CC1] | B70 |
| 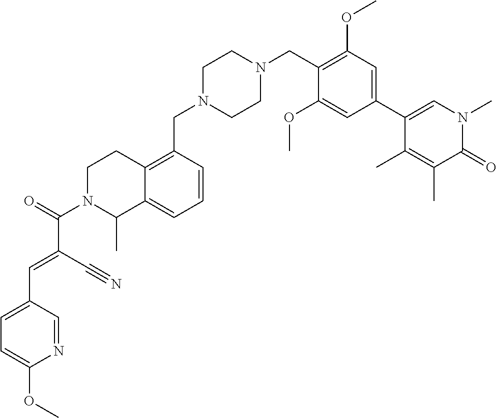 [SMILES: COc1ccc(/C=C(\C#N)C(=O)N2CCc3c(CN4CCN(Cc5c(OC)cc(-c6cn(C)c(=O)c(C)c6C)cc5OC)CC4)cccc3C2C)cn1] | B71 |
| 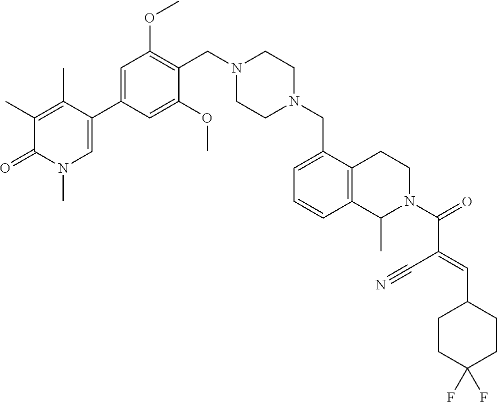 [SMILES: COc1cc(-c2cn(C)c(=O)c(C)c2C)cc(OC)c1CN1CCN(Cc2cccc3c2CCN(C(=O)/C(C#N)=C/C2CCC(F)(F)CC2)C3C)CC1] | B72 |
| 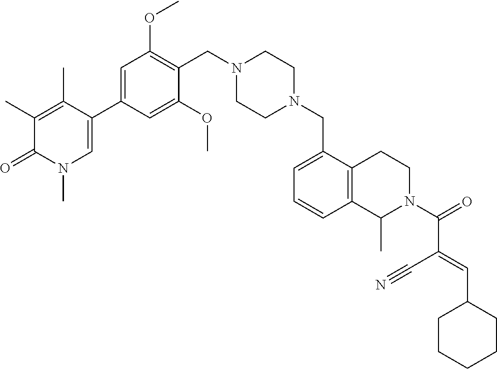 [SMILES: COc1cc(-c2cn(C)c(=O)c(C)c2C)cc(OC)c1CN1CCN(Cc2cccc3c2CCN(C(=O)/C(C#N)=C/C2CCCCC2)C3C)CC1] | B73 |
| 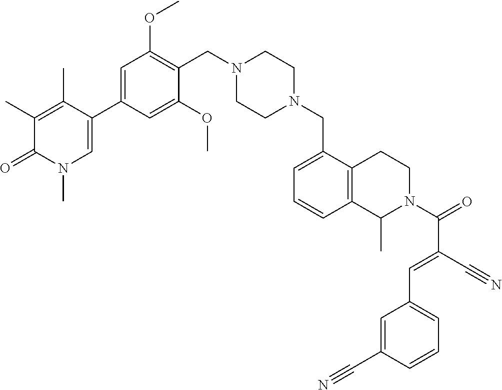 [SMILES: COc1cc(-c2cn(C)c(=O)c(C)c2C)cc(OC)c1CN1CCN(Cc2cccc3c2CCN(C(=O)/C(C#N)=C/c2cccc(C#N)c2)C3C)CC1] | B74 |
| 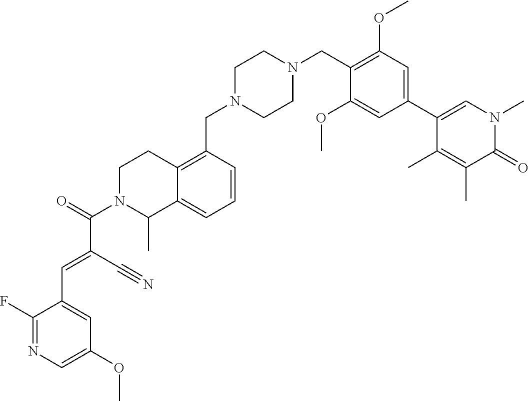 [SMILES: COc1cnc(F)c(/C=C(\C#N)C(=O)N2CCc3c(CN4CCN(Cc5c(OC)cc(-c6cn(C)c(=O)c(C)c6C)cc5OC)CC4)cccc3C2C)c1] | B75 |
| 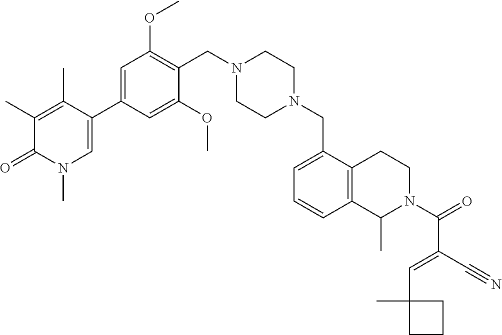 [SMILES: COc1cc(-c2cn(C)c(=O)c(C)c2C)cc(OC)c1CN1CCN(Cc2cccc3c2CCN(C(=O)/C(C#N)=C/C2(C)CCC2)C3C)CC1] | B76 |
| 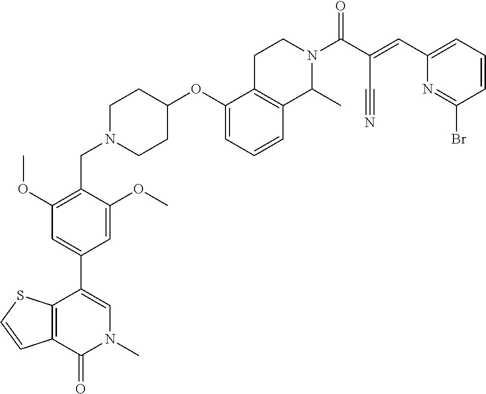 [SMILES: COc1cc(-c2cn(C)c(=O)c3ccsc23)cc(OC)c1CN1CCC(Oc2cccc3c2CCN(C(=O)/C(C#N)=C/c2cccc(Br)n2)C3C)CC1] | B77 |
| 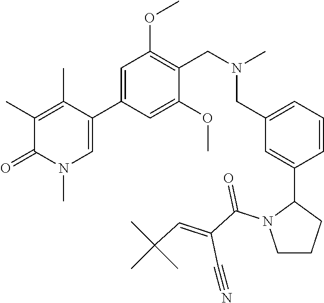 [SMILES: COc1cc(-c2cn(C)c(=O)c(C)c2C)cc(OC)c1CN(C)Cc1cccc(C2CCCN2C(=O)/C(C#N)=C/C(C)(C)C)c1] | B78 |
| 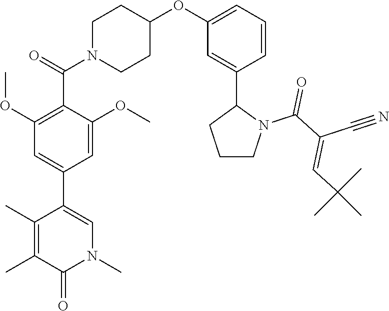 [SMILES: COc1cc(-c2cn(C)c(=O)c(C)c2C)cc(OC)c1C(=O)N1CCC(Oc2cccc(C3CCCN3C(=O)/C(C#N)=C/C(C)(C)C)c2)CC1] | B79 |
| 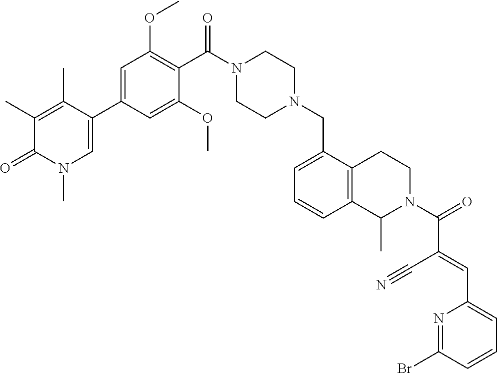 [SMILES: COc1cc(-c2cn(C)c(=O)c(C)c2C)cc(OC)c1C(=O)N1CCN(Cc2cccc3c2CCN(C(=O)/C(C#N)=C/c2cccc(Br)n2)C3C)CC1] | B80 |
| 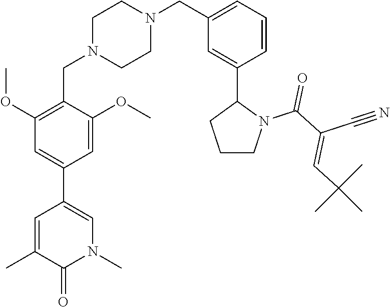 [SMILES: COc1cc(-c2cc(C)c(=O)n(C)c2)cc(OC)c1CN1CCN(Cc2cccc(C3CCCN3C(=O)/C(C#N)=C/C(C)(C)C)c2)CC1] | B81 |
| 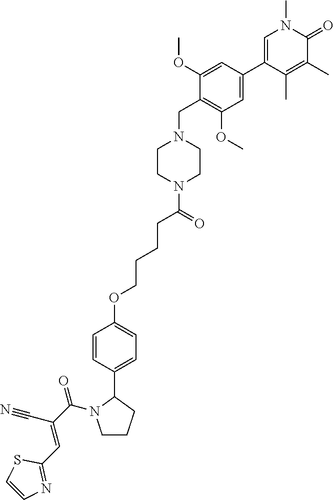 [SMILES: COc1cc(-c2cn(C)c(=O)c(C)c2C)cc(OC)c1CN1CCN(C(=O)CCCCOc2ccc(C3CCCN3C(=O)/C(C#N)=C/c3nccs3)cc2)CC1] | B82 |
| 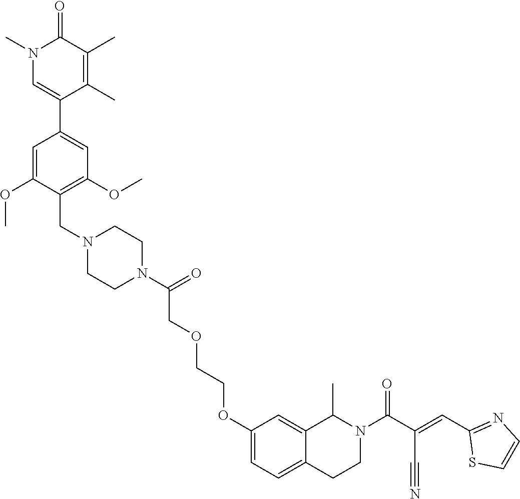 [SMILES: COc1cc(-c2cn(C)c(=O)c(C)c2C)cc(OC)c1CN1CCN(C(=O)COCCOc2ccc3c(c2)C(C)N(C(=O)/C(C#N)=C/c2nccs2)CC3)CC1] | B83 |
| 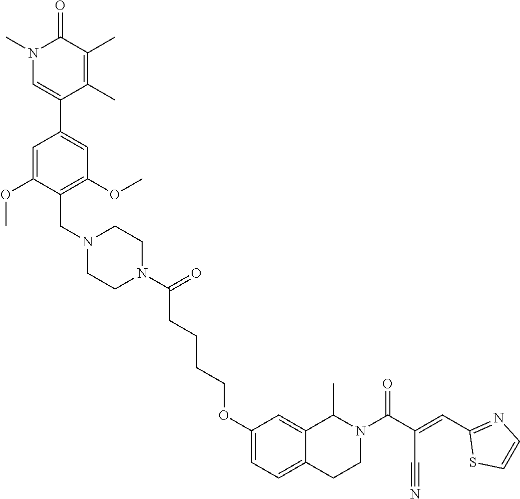 [SMILES: COc1cc(-c2cn(C)c(=O)c(C)c2C)cc(OC)c1CN1CCN(C(=O)CCCCOc2ccc3c(c2)C(C)N(C(=O)/C(C#N)=C/c2nccs2)CC3)CC1] | B84 |
| 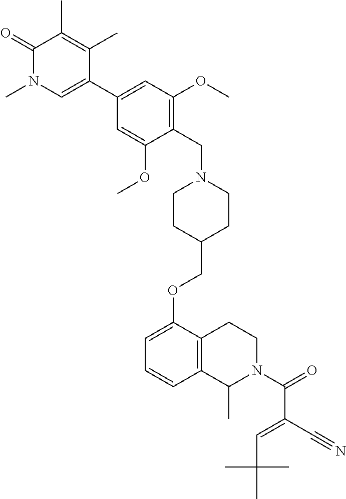 [SMILES: COc1cc(-c2cn(C)c(=O)c(C)c2C)cc(OC)c1CN1CCC(COc2cccc3c2CCN(C(=O)/C(C#N)=C/C(C)(C)C)C3C)CC1] | B86 |
| 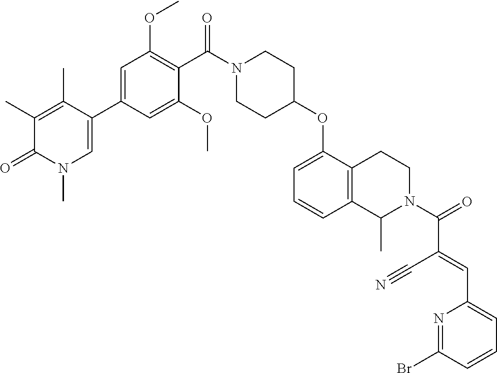 [SMILES: COc1cc(-c2cn(C)c(=O)c(C)c2C)cc(OC)c1C(=O)N1CCC(Oc2cccc3c2CCN(C(=O)/C(C#N)=C/c2cccc(Br)n2)C3C)CC1] | B88 |
| 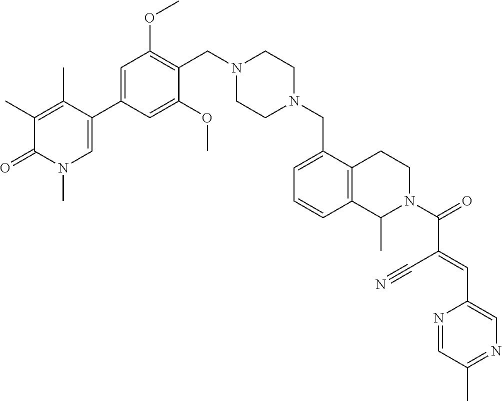 [SMILES: COc1cc(-c2cn(C)c(=O)c(C)c2C)cc(OC)c1CN1CCN(Cc2cccc3c2CCN(C(=O)/C(C#N)=C/c2cnc(C)cn2)C3C)CC1] | B89 |
| 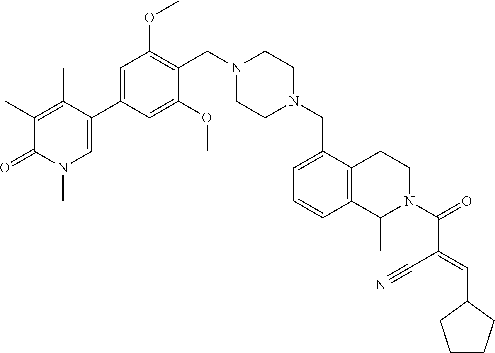 [SMILES: COc1cc(-c2cn(C)c(=O)c(C)c2C)cc(OC)c1CN1CCN(Cc2cccc3c2CCN(C(=O)/C(C#N)=C/C2CCCC2)C3C)CC1] | B90 |
| 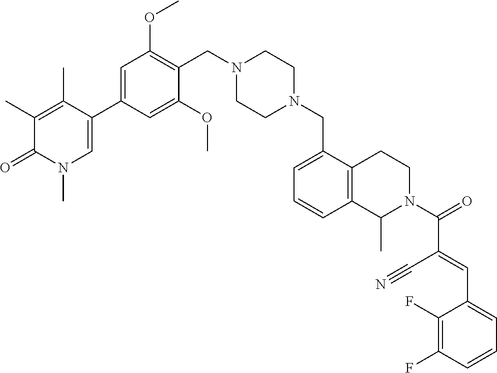 [SMILES: COc1cc(-c2cn(C)c(=O)c(C)c2C)cc(OC)c1CN1CCN(Cc2cccc3c2CCN(C(=O)/C(C#N)=C/c2cccc(F)c2F)C3C)CC1] | B91 |
| 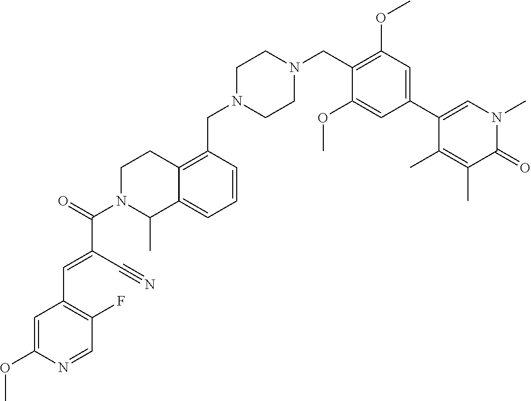 [SMILES: COc1cc(/C=C(\C#N)C(=O)N2CCc3c(CN4CCN(Cc5c(OC)cc(-c6cn(C)c(=O)c(C)c6C)cc5OC)CC4)cccc3C2C)c(F)cn1] | B92 |
| 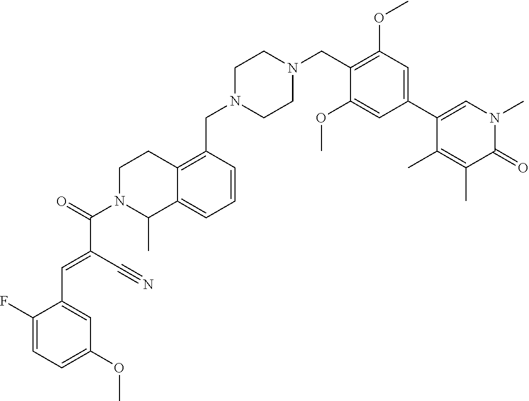 [SMILES: COc1ccc(F)c(/C=C(\C#N)C(=O)N2CCc3c(CN4CCN(Cc5c(OC)cc(-c6cn(C)c(=O)c(C)c6C)cc5OC)CC4)cccc3C2C)c1] | B93 |
| 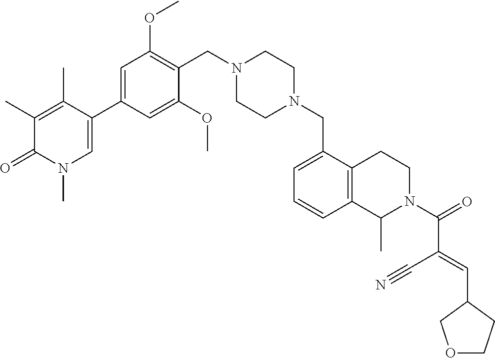 [SMILES: COc1cc(-c2cn(C)c(=O)c(C)c2C)cc(OC)c1CN1CCN(Cc2cccc3c2CCN(C(=O)/C(C#N)=C/C2CCOC2)C3C)CC1] | B94 |
| 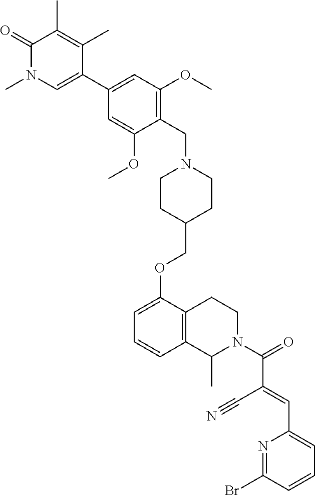 [SMILES: COc1cc(-c2cn(C)c(=O)c(C)c2C)cc(OC)c1CN1CCC(COc2cccc3c2CCN(C(=O)/C(C#N)=C/c2cccc(Br)n2)C3C)CC1] | B95 |
| 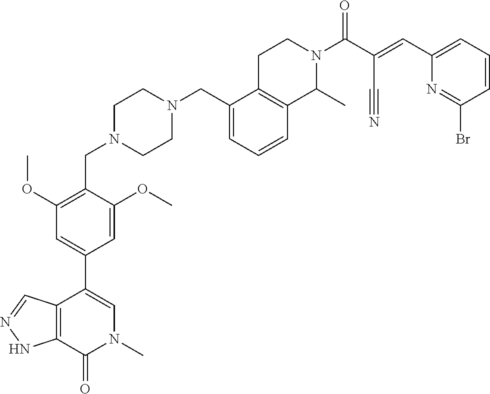 [SMILES: COc1cc(-c2cn(C)c(=O)c3[nH]ncc23)cc(OC)c1CN1CCN(Cc2cccc3c2CCN(C(=O)/C(C#N)=C/c2cccc(Br)n2)C3C)CC1] | B96 |
| 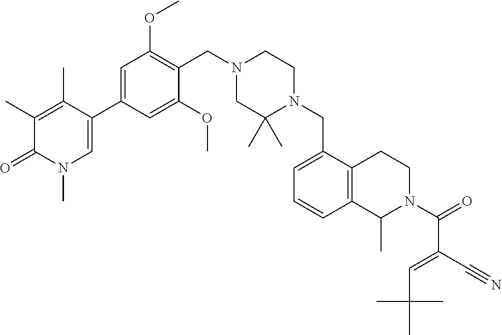 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)C(C)(C)C1] | B98 |
| 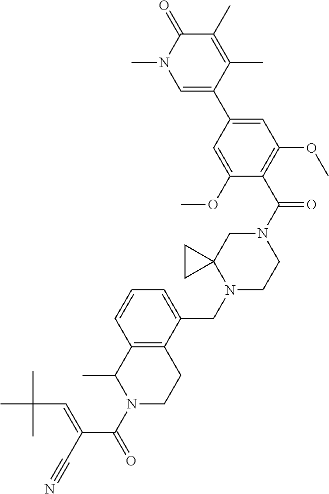 [SMILES: COc1cc(-c2cn(C)c(=O)c(C)c2C)cc(OC)c1C(=O)N1CCN(Cc2cccc3c2CCN(C(=O)/C(C#N)=C/C(C)(C)C)C3C)C2(CC2)C1] | B99 |
| 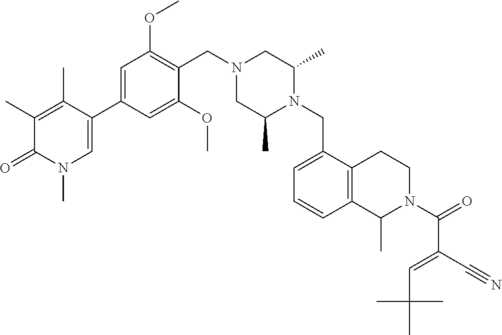 [SMILES: COc1cc(-c2cn(C)c(=O)c(C)c2C)cc(OC)c1CN1C[C@H](C)N(Cc2cccc3c2CCN(C(=O)/C(C#N)=C/C(C)(C)C)C3C)[C@@H](C)C1] | B100 |
| 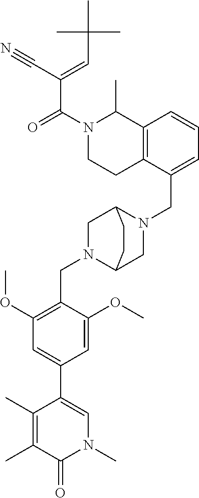 [SMILES: COc1cc(-c2cn(C)c(=O)c(C)c2C)cc(OC)c1CN1CC2CCC1CN2Cc1cccc2c1CCN(C(=O)/C(C#N)=C/C(C)(C)C)C2C] | B101 |
| 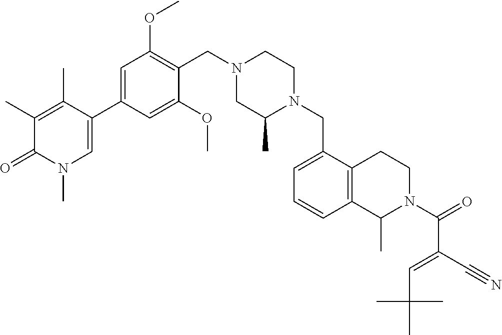 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)[C@@H](C)C1] | B102 |
| 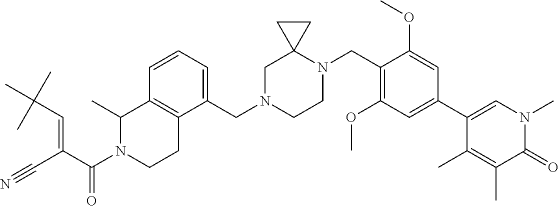 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)CC12CC2] | B103 |
| 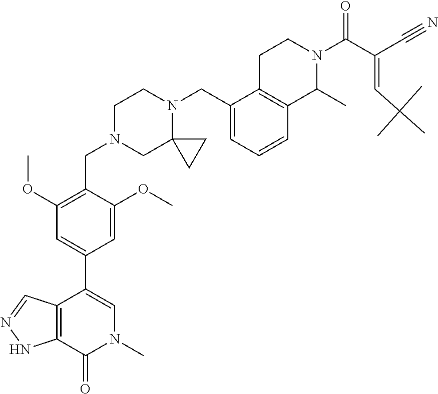 [SMILES: COc1cc(-c2cn(C)c(=O)c3[nH]ncc23)cc(OC)c1CN1CCN(Cc2cccc3c2CCN(C(=O)/C(C#N)=C/C(C)(C)C)C3C)C2(CC2)C1] | B104 |
| 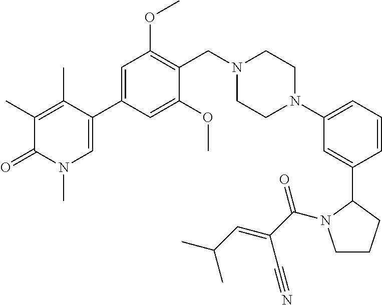 [SMILES: COc1cc(-c2cn(C)c(=O)c(C)c2C)cc(OC)c1CN1CCN(c2cccc(C3CCCN3C(=O)/C(C#N)=C/C(C)C)c2)CC1] | B106 |
| 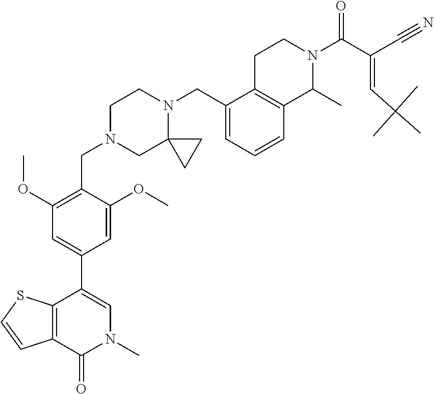 [SMILES: COc1cc(-c2cn(C)c(=O)c3ccsc23)cc(OC)c1CN1CCN(Cc2cccc3c2CCN(C(=O)/C(C#N)=C/C(C)(C)C)C3C)C2(CC2)C1] | B107 |
| 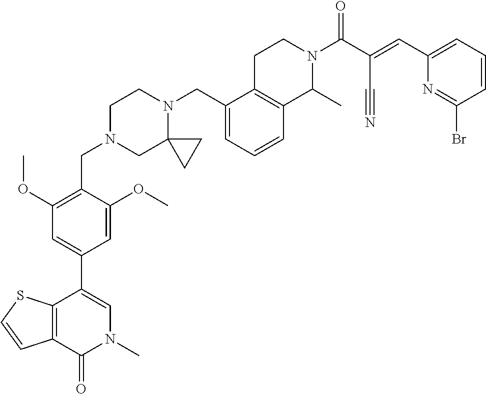 [SMILES: COc1cc(-c2cn(C)c(=O)c3ccsc23)cc(OC)c1CN1CCN(Cc2cccc3c2CCN(C(=O)/C(C#N)=C/c2cccc(Br)n2)C3C)C2(CC2)C1] | B108 |
| 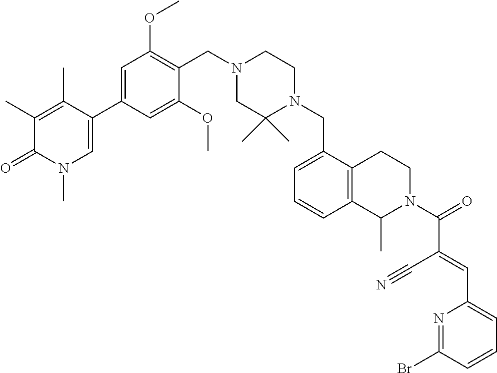 [SMILES: COc1cc(-c2cn(C)c(=O)c(C)c2C)cc(OC)c1CN1CCN(Cc2cccc3c2CCN(C(=O)/C(C#N)=C/c2cccc(Br)n2)C3C)C(C)(C)C1] | B109 |
| 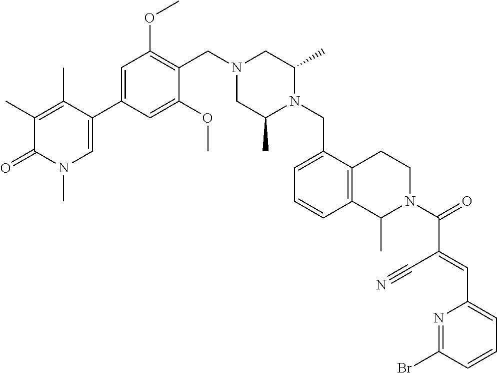 [SMILES: COc1cc(-c2cn(C)c(=O)c(C)c2C)cc(OC)c1CN1C[C@H](C)N(Cc2cccc3c2CCN(C(=O)/C(C#N)=C/c2cccc(Br)n2)C3C)[C@@H](C)C1] | B110 |
| 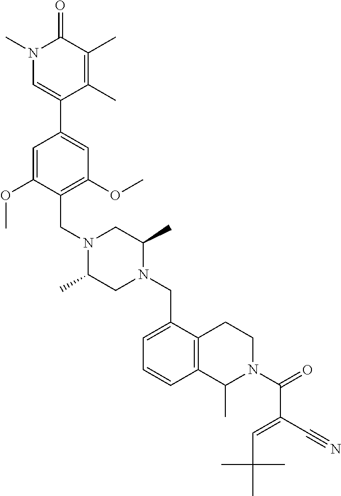 [SMILES: COc1cc(-c2cn(C)c(=O)c(C)c2C)cc(OC)c1CN1C[C@@H](C)N(Cc2cccc3c2CCN(C(=O)/C(C#N)=C/C(C)(C)C)C3C)C[C@@H]1C] | B111 |
| 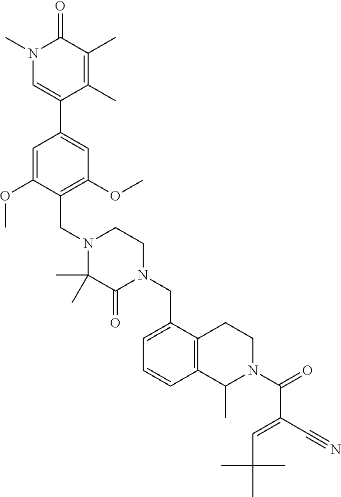 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)C(=O)C1(C)C] | B112 |
| 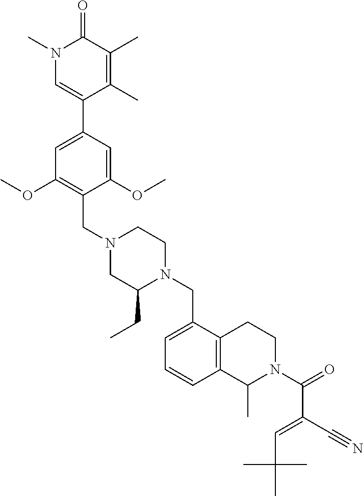 [SMILES: CC[C@H]1CN(Cc2c(OC)cc(-c3cn(C)c(=O)c(C)c3C)cc2OC)CCN1Cc1cccc2c1CCN(C(=O)/C(C#N)=C/C(C)(C)C)C2C] | B113 |
| 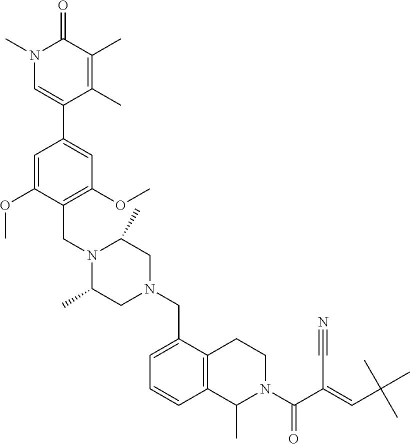 [SMILES: COc1cc(-c2cn(C)c(=O)c(C)c2C)cc(OC)c1CN1[C@H](C)CN(Cc2cccc3c2CCN(C(=O)/C(C#N)=C/C(C)(C)C)C3C)C[C@@H]1C] | B114 |
| 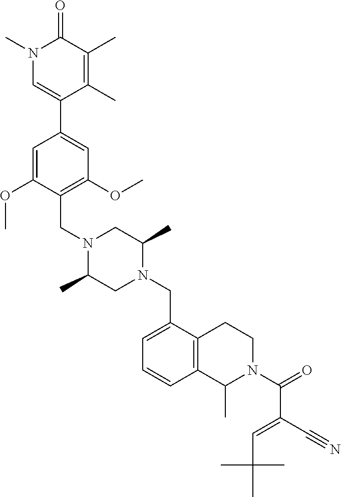 [SMILES: COc1cc(-c2cn(C)c(=O)c(C)c2C)cc(OC)c1CN1C[C@@H](C)N(Cc2cccc3c2CCN(C(=O)/C(C#N)=C/C(C)(C)C)C3C)C[C@H]1C] | B115 |
| 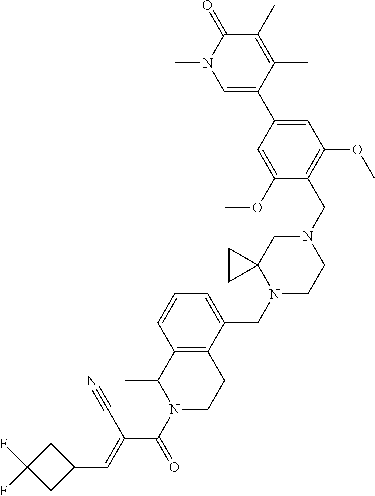 [SMILES: COc1cc(-c2cn(C)c(=O)c(C)c2C)cc(OC)c1CN1CCN(Cc2cccc3c2CCN(C(=O)/C(C#N)=C/C2CC(F)(F)C2)C3C)C2(CC2)C1] | B116 |
| 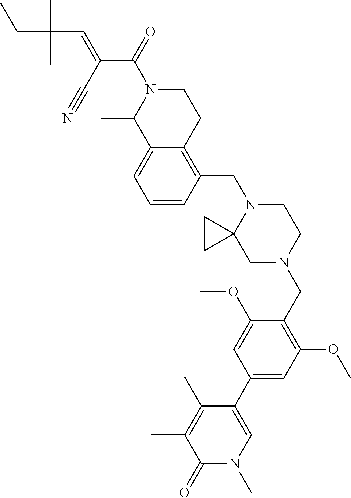 [SMILES: CCC(C)(C)/C=C(\C#N)C(=O)N1CCc2c(CN3CCN(Cc4c(OC)cc(-c5cn(C)c(=O)c(C)c5C)cc4OC)CC34CC4)cccc2C1C] | B117 |
| 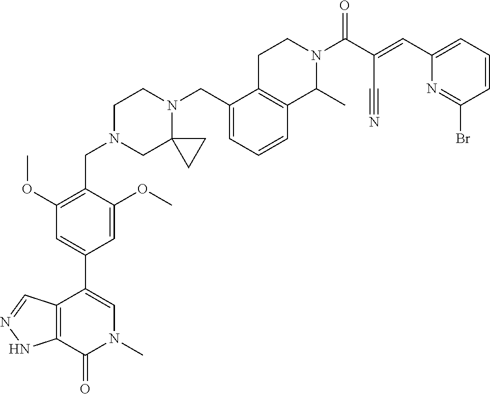 [SMILES: COc1cc(-c2cn(C)c(=O)c3[nH]ncc23)cc(OC)c1CN1CCN(Cc2cccc3c2CCN(C(=O)/C(C#N)=C/c2cccc(Br)n2)C3C)C2(CC2)C1] | B118 |
| 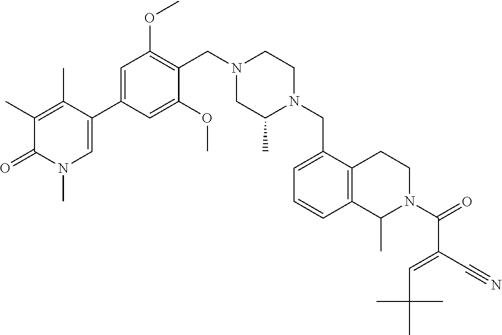 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)[C@H](C)C1] | B119 |
| 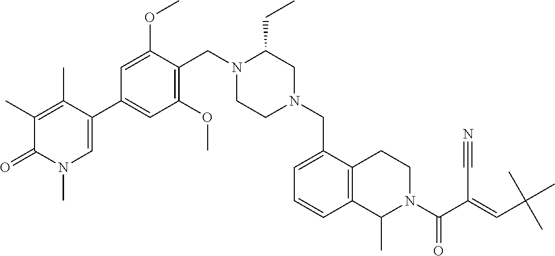 [SMILES: CC[C@@H]1CN(Cc2cccc3c2CCN(C(=O)/C(C#N)=C/C(C)(C)C)C3C)CCN1Cc1c(OC)cc(-c2cn(C)c(=O)c(C)c2C)cc1OC] | B120 |
| 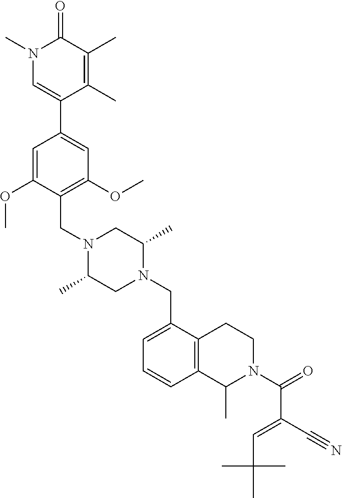 [SMILES: COc1cc(-c2cn(C)c(=O)c(C)c2C)cc(OC)c1CN1C[C@H](C)N(Cc2cccc3c2CCN(C(=O)/C(C#N)=C/C(C)(C)C)C3C)C[C@@H]1C] | B121 |
| 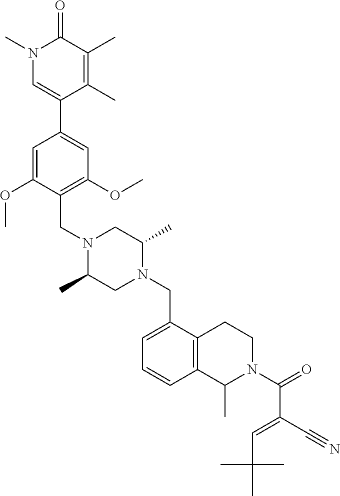 [SMILES: COc1cc(-c2cn(C)c(=O)c(C)c2C)cc(OC)c1CN1C[C@H](C)N(Cc2cccc3c2CCN(C(=O)/C(C#N)=C/C(C)(C)C)C3C)C[C@H]1C] | B122 |
| 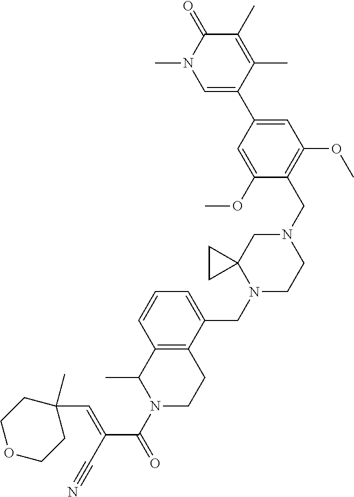 [SMILES: COc1cc(-c2cn(C)c(=O)c(C)c2C)cc(OC)c1CN1CCN(Cc2cccc3c2CCN(C(=O)/C(C#N)=C/C2(C)CCOCC2)C3C)C2(CC2)C1] | B123 |
| 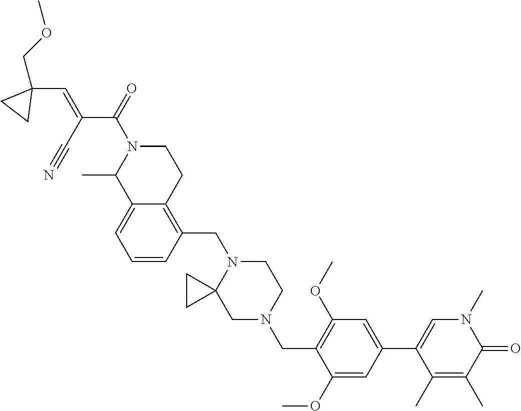 [SMILES: COCC1(/C=C(\C#N)C(=O)N2CCc3c(CN4CCN(Cc5c(OC)cc(-c6cn(C)c(=O)c(C)c6C)cc5OC)CC45CC5)cccc3C2C)CC1] | B124 |
| 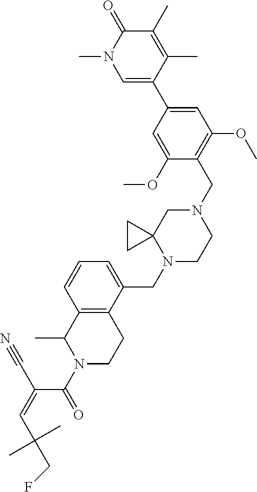 [SMILES: COc1cc(-c2cn(C)c(=O)c(C)c2C)cc(OC)c1CN1CCN(Cc2cccc3c2CCN(C(=O)/C(C#N)=C\C(C)(C)CF)C3C)C2(CC2)C1] | B125 |
| 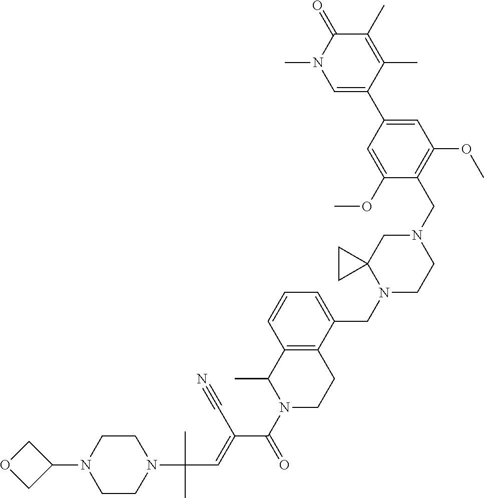 [SMILES: COc1cc(-c2cn(C)c(=O)c(C)c2C)cc(OC)c1CN1CCN(Cc2cccc3c2CCN(C(=O)/C(C#N)=C/C(C)(C)N2CCN(C4COC4)CC2)C3C)C2(CC2)C1] | B126 |
| 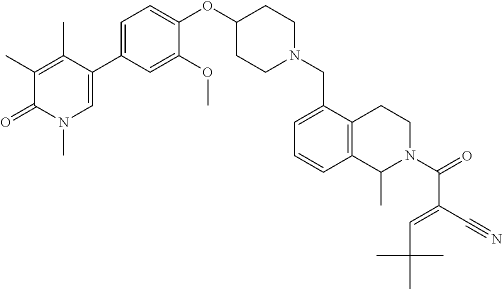 [SMILES: COc1cc(-c2cn(C)c(=O)c(C)c2C)ccc1OC1CCN(Cc2cccc3c2CCN(C(=O)/C(C#N)=C/C(C)(C)C)C3C)CC1] | B127 |
| 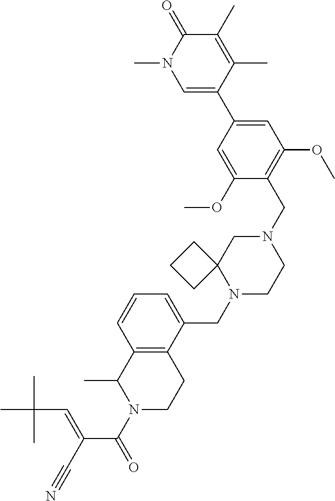 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)C2(CCC2)C1] | B130 |
| 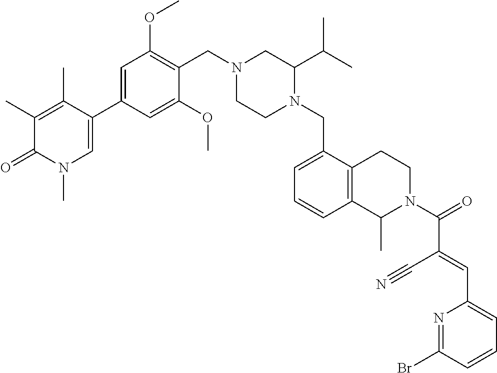 [SMILES: COc1cc(-c2cn(C)c(=O)c(C)c2C)cc(OC)c1CN1CCN(Cc2cccc3c2CCN(C(=O)/C(C#N)=C/c2cccc(Br)n2)C3C)C(C(C)C)C1] | B131 |
| 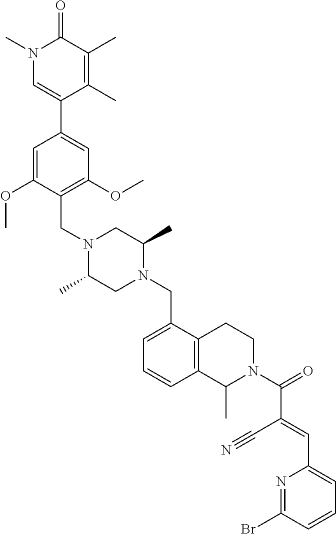 [SMILES: COc1cc(-c2cn(C)c(=O)c(C)c2C)cc(OC)c1CN1C[C@@H](C)N(Cc2cccc3c2CCN(C(=O)/C(C#N)=C/c2cccc(Br)n2)C3C)C[C@@H]1C] | B132 |
| 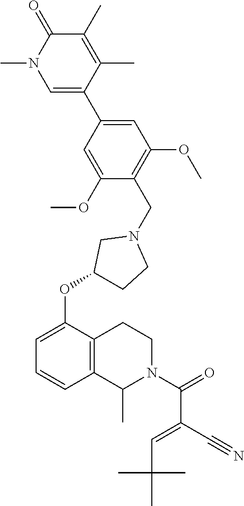 [SMILES: COc1cc(-c2cn(C)c(=O)c(C)c2C)cc(OC)c1CN1CC[C@H](Oc2cccc3c2CCN(C(=O)/C(C#N)=C/C(C)(C)C)C3C)C1] | B133 |
| 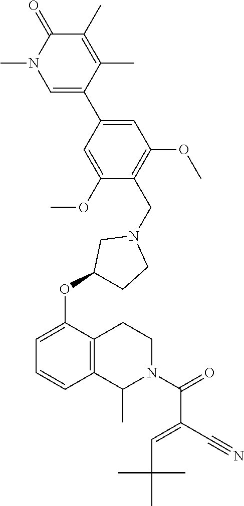 [SMILES: COc1cc(-c2cn(C)c(=O)c(C)c2C)cc(OC)c1CN1CC[C@@H](Oc2cccc3c2CCN(C(=O)/C(C#N)=C/C(C)(C)C)C3C)C1] | B134 |
| 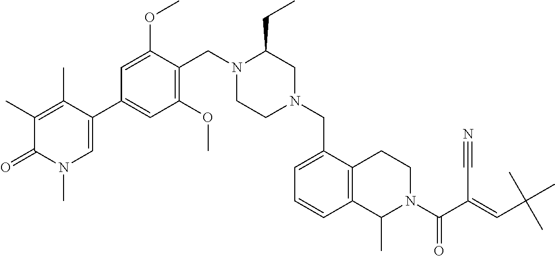 [SMILES: CC[C@H]1CN(Cc2cccc3c2CCN(C(=O)/C(C#N)=C/C(C)(C)C)C3C)CCN1Cc1c(OC)cc(-c2cn(C)c(=O)c(C)c2C)cc1OC] | B135 |
| 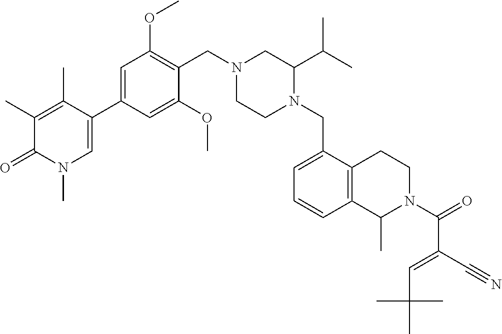 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)C(C(C)C)C1] | B136 |
| 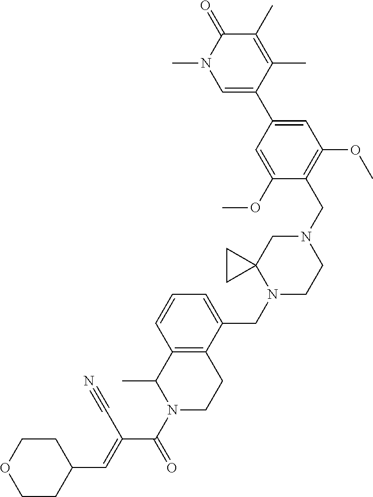 [SMILES: COc1cc(-c2cn(C)c(=O)c(C)c2C)cc(OC)c1CN1CCN(Cc2cccc3c2CCN(C(=O)/C(C#N)=C/C2CCOCC2)C3C)C2(CC2)C1] | B137 |
| 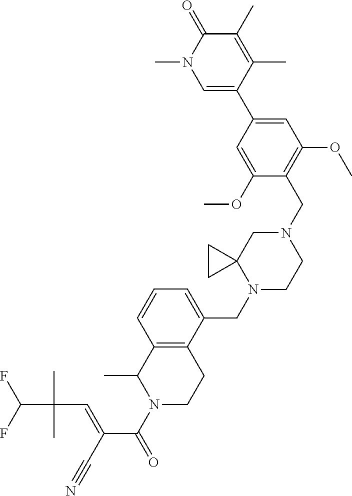 [SMILES: COc1cc(-c2cn(C)c(=O)c(C)c2C)cc(OC)c1CN1CCN(Cc2cccc3c2CCN(C(=O)/C(C#N)=C/C(C)(C)C(F)F)C3C)C2(CC2)C1] | B138 |
| 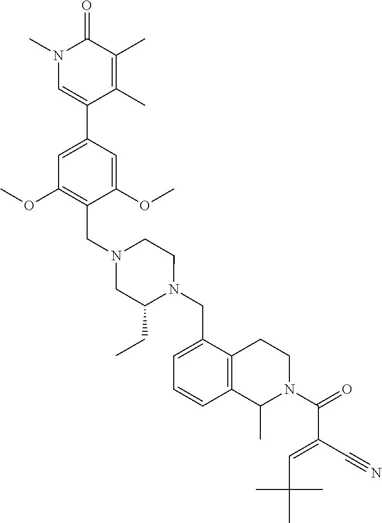 [SMILES: CC[C@@H]1CN(Cc2c(OC)cc(-c3cn(C)c(=O)c(C)c3C)cc2OC)CCN1Cc1cccc2c1CCN(C(=O)/C(C#N)=C/C(C)(C)C)C2C] | B139 |
| 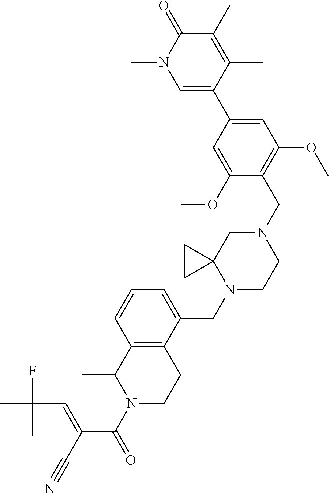 [SMILES: COc1cc(-c2cn(C)c(=O)c(C)c2C)cc(OC)c1CN1CCN(Cc2cccc3c2CCN(C(=O)/C(C#N)=C/C(C)(C)F)C3C)C2(CC2)C1] | B140 |
| 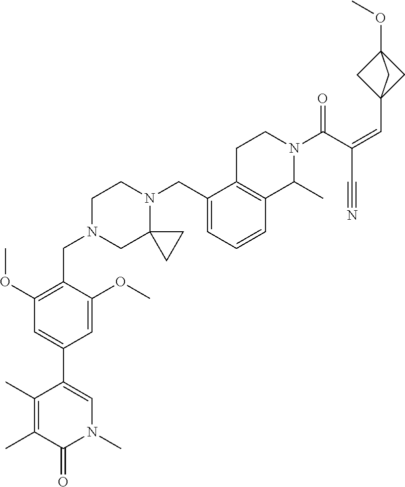 [SMILES: COc1cc(-c2cn(C)c(=O)c(C)c2C)cc(OC)c1CN1CCN(Cc2cccc3c2CCN(C(=O)/C(C#N)=C\C24CC(OC)(C2)C4)C3C)C2(CC2)C1] | B141 |
| 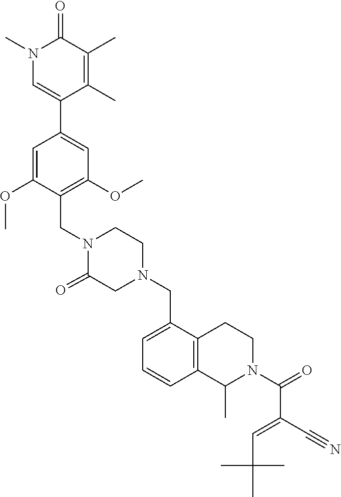 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)CC1=O] | B142 |
| 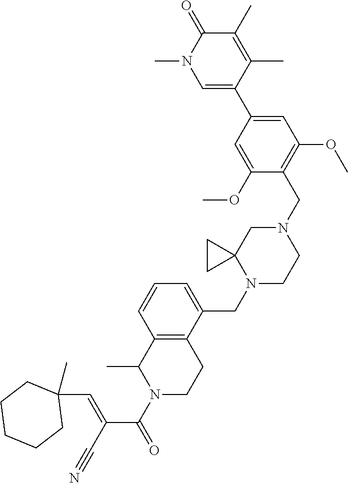 [SMILES: COc1cc(-c2cn(C)c(=O)c(C)c2C)cc(OC)c1CN1CCN(Cc2cccc3c2CCN(C(=O)/C(C#N)=C/C2(C)CCCCC2)C3C)C2(CC2)C1] | B143 |
| 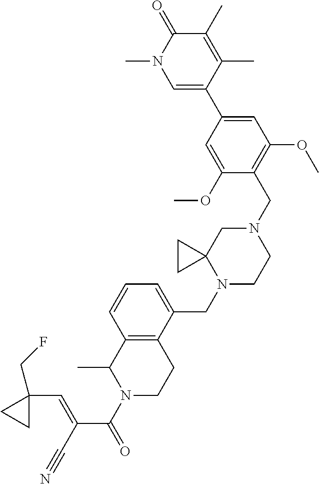 [SMILES: COc1cc(-c2cn(C)c(=O)c(C)c2C)cc(OC)c1CN1CCN(Cc2cccc3c2CCN(C(=O)/C(C#N)=C/C2(CF)CC2)C3C)C2(CC2)C1] | B144 |
| 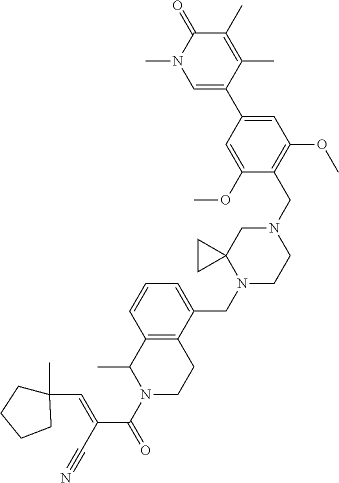 [SMILES: COc1cc(-c2cn(C)c(=O)c(C)c2C)cc(OC)c1CN1CCN(Cc2cccc3c2CCN(C(=O)/C(C#N)=C/C2(C)CCCC2)C3C)C2(CC2)C1] | B145 |
| 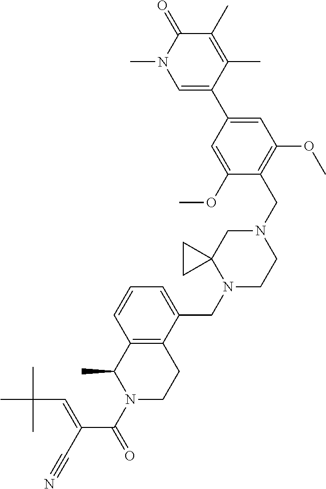 [SMILES: COc1cc(-c2cn(C)c(=O)c(C)c2C)cc(OC)c1CN1CCN(Cc2cccc3c2CCN(C(=O)/C(C#N)=C/C(C)(C)C)[C@H]3C)C2(CC2)C1] | B146 |
| 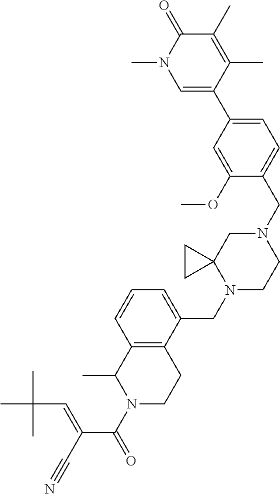 [SMILES: COc1cc(-c2cn(C)c(=O)c(C)c2C)ccc1CN1CCN(Cc2cccc3c2CCN(C(=O)/C(C#N)=C/C(C)(C)C)C3C)C2(CC2)C1] | B147 |
| 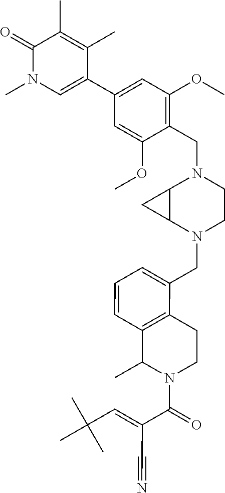 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)C2CC21] | B148 |
| 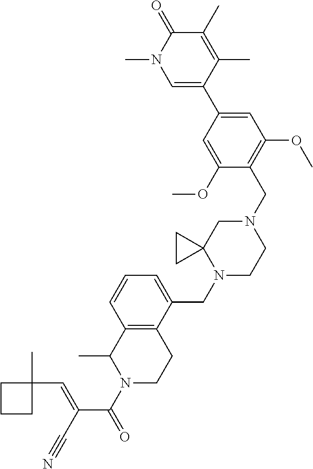 [SMILES: COc1cc(-c2cn(C)c(=O)c(C)c2C)cc(OC)c1CN1CCN(Cc2cccc3c2CCN(C(=O)/C(C#N)=C/C2(C)CCC2)C3C)C2(CC2)C1] | B149 |
| 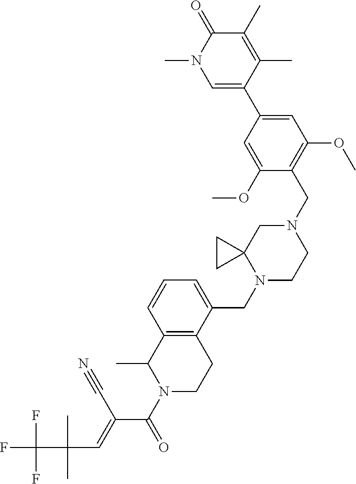 [SMILES: COc1cc(-c2cn(C)c(=O)c(C)c2C)cc(OC)c1CN1CCN(Cc2cccc3c2CCN(C(=O)/C(C#N)=C/C(C)(C)C(F)(F)F)C3C)C2(CC2)C1] | B152 |
| 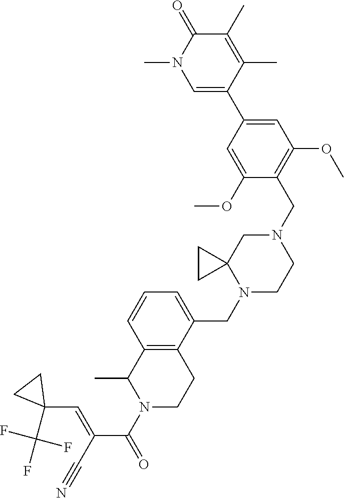 [SMILES: COc1cc(-c2cn(C)c(=O)c(C)c2C)cc(OC)c1CN1CCN(Cc2cccc3c2CCN(C(=O)/C(C#N)=C/C2(C(F)(F)F)CC2)C3C)C2(CC2)C1] | B153 |
| 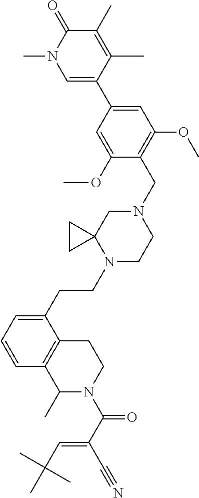 [SMILES: COc1cc(-c2cn(C)c(=O)c(C)c2C)cc(OC)c1CN1CCN(CCc2cccc3c2CCN(C(=O)/C(C#N)=C/C(C)(C)C)C3C)C2(CC2)C1] | B154 |
| 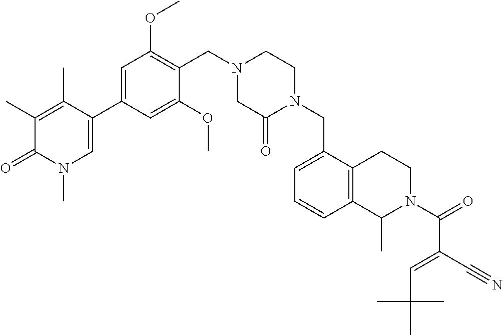 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)C(=O)C1] | B155 |
| 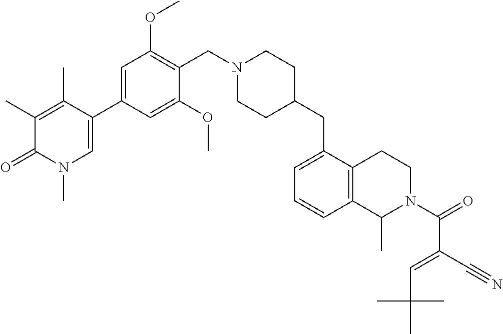 [SMILES: COc1cc(-c2cn(C)c(=O)c(C)c2C)cc(OC)c1CN1CCC(Cc2cccc3c2CCN(C(=O)/C(C#N)=C/C(C)(C)C)C3C)CC1] | B156 |
| 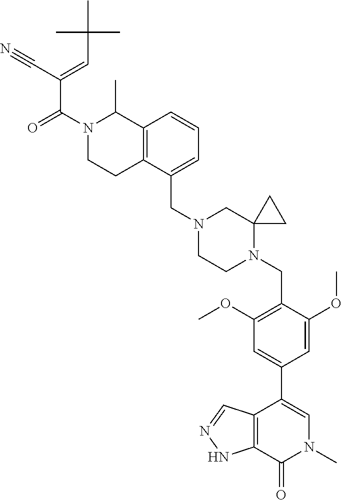 [SMILES: COc1cc(-c2cn(C)c(=O)c3[nH]ncc23)cc(OC)c1CN1CCN(Cc2cccc3c2CCN(C(=O)/C(C#N)=C/C(C)(C)C)C3C)CC12CC2] | B158 |
| 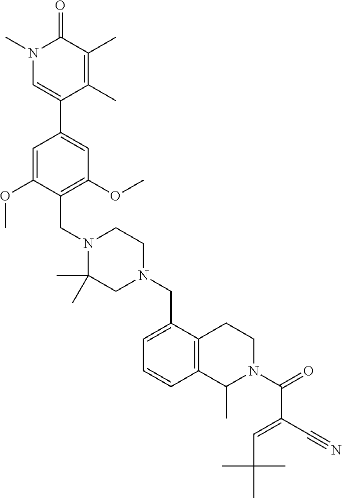 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)CC1(C)C] | B159 |
| 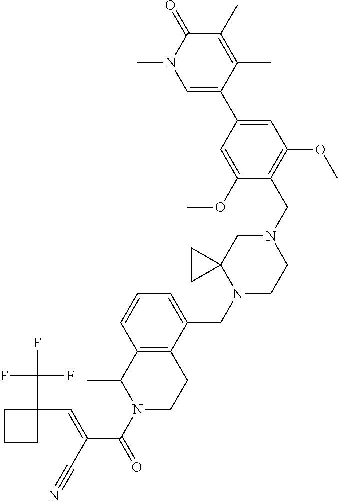 [SMILES: COc1cc(-c2cn(C)c(=O)c(C)c2C)cc(OC)c1CN1CCN(Cc2cccc3c2CCN(C(=O)/C(C#N)=C/C2(C(F)(F)F)CCC2)C3C)C2(CC2)C1] | B160 |
| 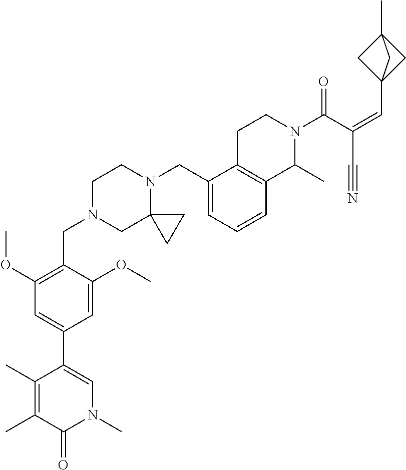 [SMILES: COc1cc(-c2cn(C)c(=O)c(C)c2C)cc(OC)c1CN1CCN(Cc2cccc3c2CCN(C(=O)/C(C#N)=C\C24CC(C)(C2)C4)C3C)C2(CC2)C1] | B161 |
| 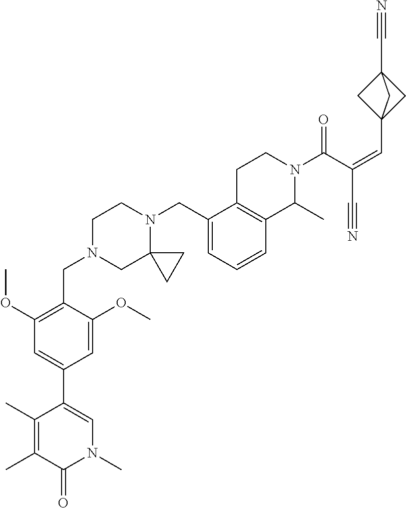 [SMILES: COc1cc(-c2cn(C)c(=O)c(C)c2C)cc(OC)c1CN1CCN(Cc2cccc3c2CCN(C(=O)/C(C#N)=C\C24CC(C#N)(C2)C4)C3C)C2(CC2)C1] | B162 |
| 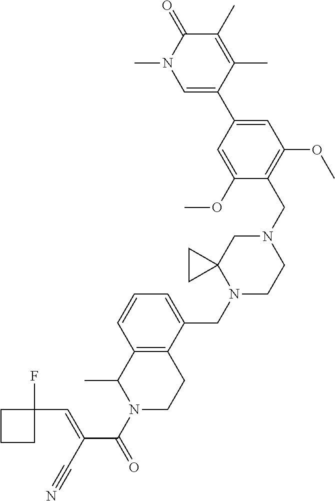 [SMILES: COc1cc(-c2cn(C)c(=O)c(C)c2C)cc(OC)c1CN1CCN(Cc2cccc3c2CCN(C(=O)/C(C#N)=C/C2(F)CCC2)C3C)C2(CC2)C1] | B164 |
| 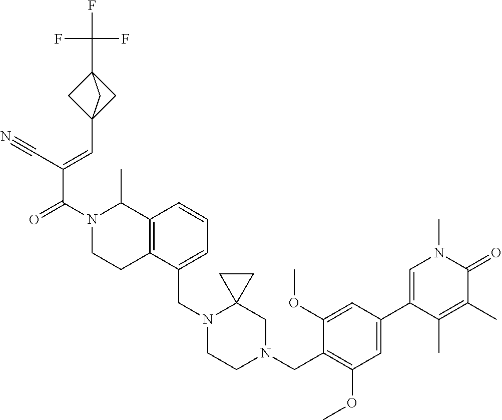 [SMILES: COc1cc(-c2cn(C)c(=O)c(C)c2C)cc(OC)c1CN1CCN(Cc2cccc3c2CCN(C(=O)/C(C#N)=C/C24CC(C(F)(F)F)(C2)C4)C3C)C2(CC2)C1] | B165 |
| 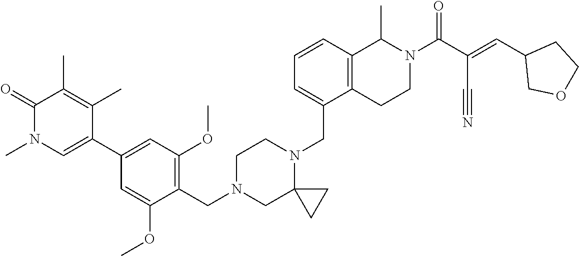 [SMILES: COc1cc(-c2cn(C)c(=O)c(C)c2C)cc(OC)c1CN1CCN(Cc2cccc3c2CCN(C(=O)/C(C#N)=C/C2CCOC2)C3C)C2(CC2)C1] | B169 |
| 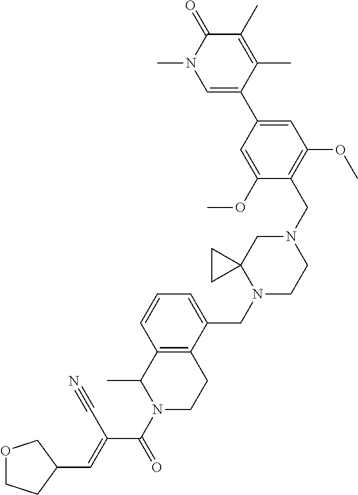 [SMILES: COc1cc(-c2cn(C)c(=O)c(C)c2C)cc(OC)c1CN1CCN(Cc2cccc3c2CCN(C(=O)/C(C#N)=C/C2CCOC2)C3C)C2(CC2)C1] | B173 |
| 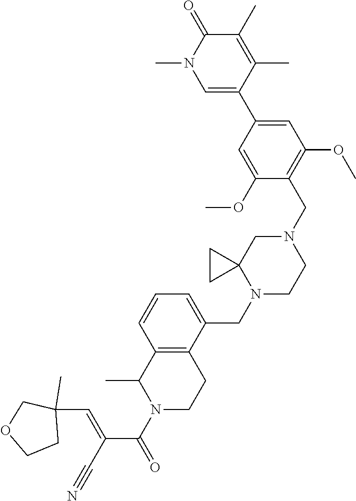 [SMILES: COc1cc(-c2cn(C)c(=O)c(C)c2C)cc(OC)c1CN1CCN(Cc2cccc3c2CCN(C(=O)/C(C#N)=C/C2(C)CCOC2)C3C)C2(CC2)C1] | B174 |
| 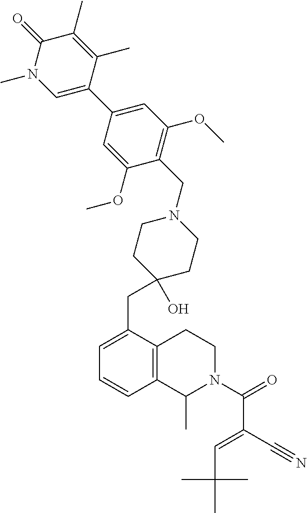 [SMILES: COc1cc(-c2cn(C)c(=O)c(C)c2C)cc(OC)c1CN1CCC(O)(Cc2cccc3c2CCN(C(=O)/C(C#N)=C/C(C)(C)C)C3C)CC1] | B175 |
| 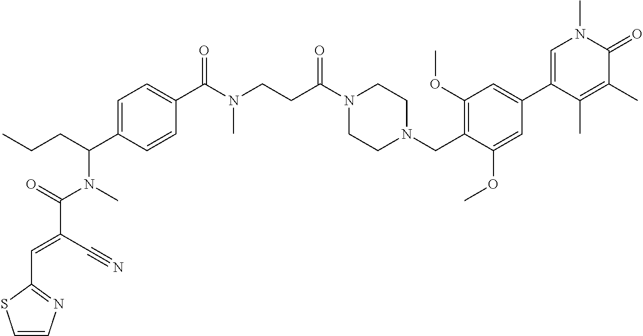 [SMILES: CCCC(c1ccc(C(=O)N(C)CCC(=O)N2CCN(Cc3c(OC)cc(-c4cn(C)c(=O)c(C)c4C)cc3OC)CC2)cc1)N(C)C(=O)/C(C#N)=C/c1nccs1] | B180 |
| 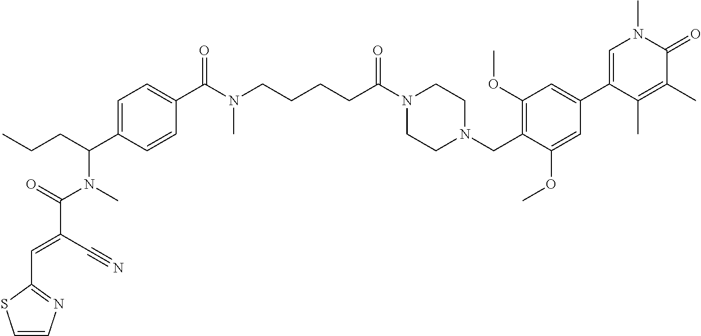 [SMILES: CCCC(c1ccc(C(=O)N(C)CCCCC(=O)N2CCN(Cc3c(OC)cc(-c4cn(C)c(=O)c(C)c4C)cc3OC)CC2)cc1)N(C)C(=O)/C(C#N)=C/c1nccs1] | B181 |
| 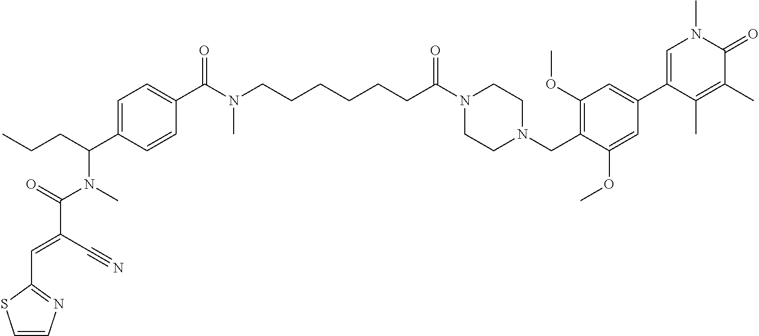 [SMILES: CCCC(c1ccc(C(=O)N(C)CCCCCCC(=O)N2CCN(Cc3c(OC)cc(-c4cn(C)c(=O)c(C)c4C)cc3OC)CC2)cc1)N(C)C(=O)/C(C#N)=C/c1nccs1] | B182 |
| 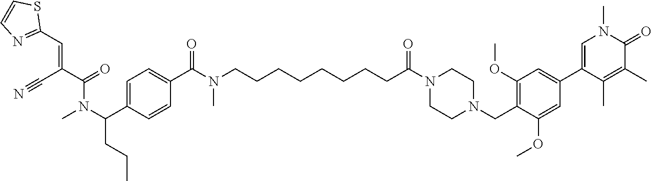 [SMILES: CCCC(c1ccc(C(=O)N(C)CCCCCCCCC(=O)N2CCN(Cc3c(OC)cc(-c4cn(C)c(=O)c(C)c4C)cc3OC)CC2)cc1)N(C)C(=O)/C(C#N)=C/c1nccs1] | B183 |
| 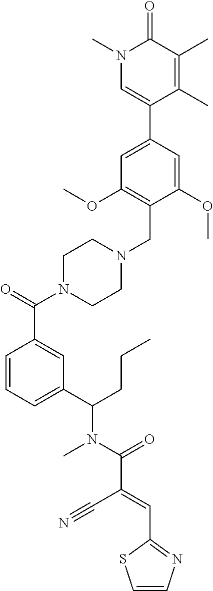 [SMILES: CCCC(c1cccc(C(=O)N2CCN(Cc3c(OC)cc(-c4cn(C)c(=O)c(C)c4C)cc3OC)CC2)c1)N(C)C(=O)/C(C#N)=C/c1nccs1] | B184 |
| 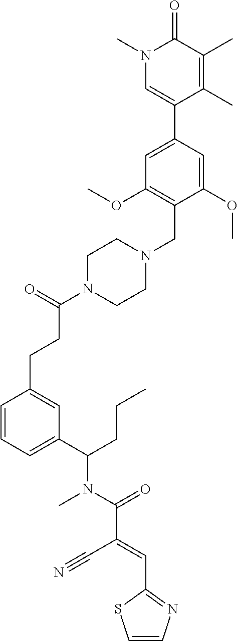 [SMILES: CCCC(c1cccc(CCC(=O)N2CCN(Cc3c(OC)cc(-c4cn(C)c(=O)c(C)c4C)cc3OC)CC2)c1)N(C)C(=O)/C(C#N)=C/c1nccs1] | B185 |
| 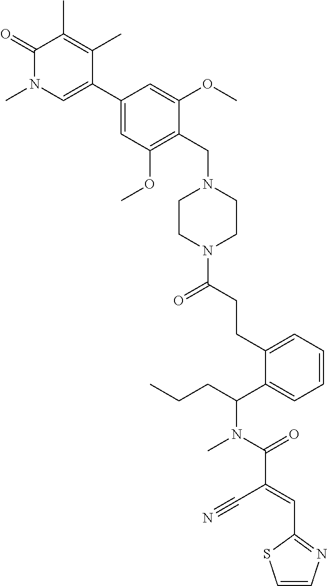 [SMILES: CCCC(c1ccccc1CCC(=O)N1CCN(Cc2c(OC)cc(-c3cn(C)c(=O)c(C)c3C)cc2OC)CC1)N(C)C(=O)/C(C#N)=C/c1nccs1] | B186 |
| 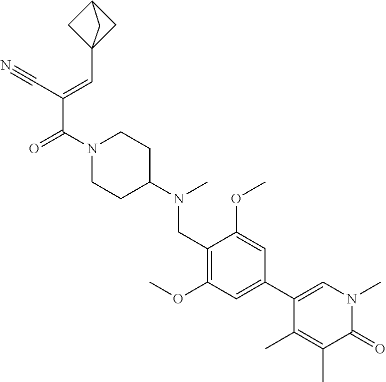 [SMILES: COc1cc(-c2cn(C)c(=O)c(C)c2C)cc(OC)c1CN(C)C1CCN(C(=O)/C(C#N)=C/C23CC(C2)C3)CC1] | B187 |
| 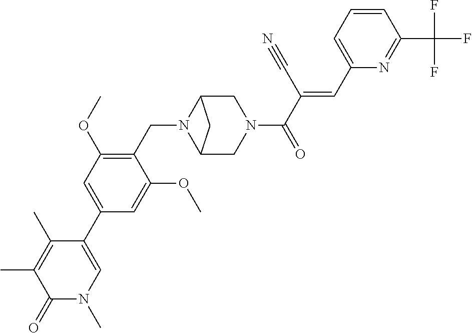 [SMILES: COc1cc(-c2cn(C)c(=O)c(C)c2C)cc(OC)c1CN1C2CC1CN(C(=O)/C(C#N)=C/c1cccc(C(F)(F)F)n1)C2] | B188 |
| 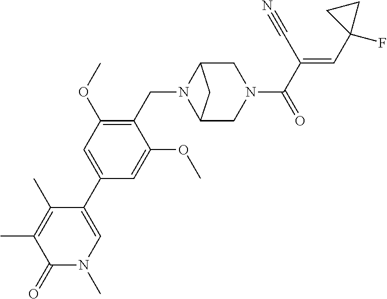 [SMILES: COc1cc(-c2cn(C)c(=O)c(C)c2C)cc(OC)c1CN1C2CC1CN(C(=O)/C(C#N)=C/C1(F)CC1)C2] | B189 |
| 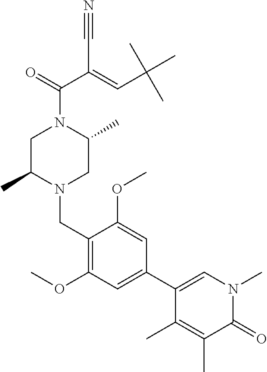 [SMILES: COc1cc(-c2cn(C)c(=O)c(C)c2C)cc(OC)c1CN1C[C@@H](C)N(C(=O)/C(C#N)=C/C(C)(C)C)C[C@@H]1C] | B190 |
| 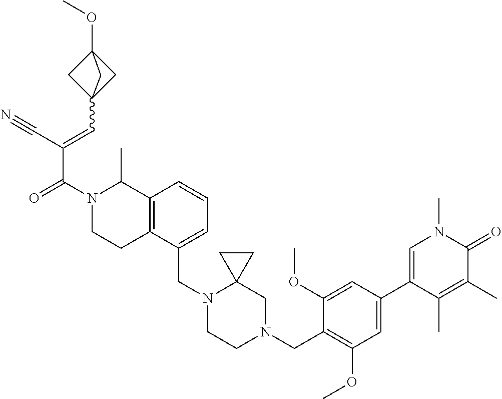 [SMILES: COc1cc(-c2cn(C)c(=O)c(C)c2C)cc(OC)c1CN1CCN(Cc2cccc3c2CCN(C(=O)C(C#N)=CC24CC(OC)(C2)C4)C3C)C2(CC2)C1] | B191 |
| 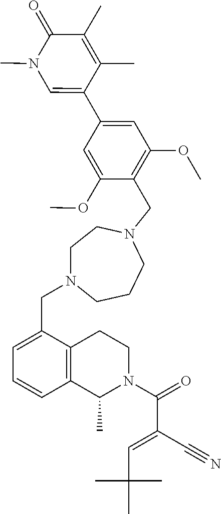 [SMILES: COc1cc(-c2cn(C)c(=O)c(C)c2C)cc(OC)c1CN1CCCN(Cc2cccc3c2CCN(C(=O)/C(C#N)=C/C(C)(C)C)[C@@H]3C)CC1] | B192 |
| 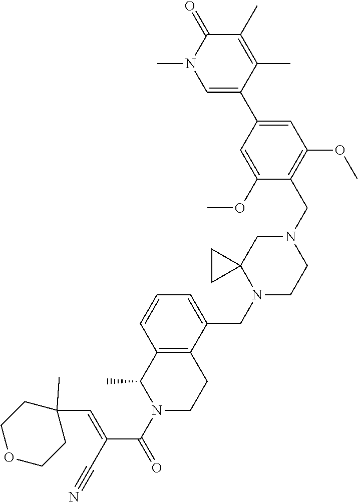 [SMILES: COc1cc(-c2cn(C)c(=O)c(C)c2C)cc(OC)c1CN1CCN(Cc2cccc3c2CCN(C(=O)/C(C#N)=C/C2(C)CCOCC2)[C@@H]3C)C2(CC2)C1] | B193 |
| 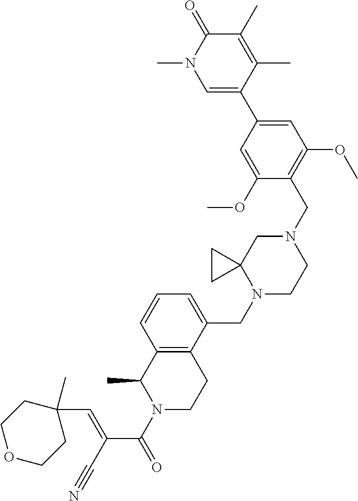 [SMILES: COc1cc(-c2cn(C)c(=O)c(C)c2C)cc(OC)c1CN1CCN(Cc2cccc3c2CCN(C(=O)/C(C#N)=C/C2(C)CCOCC2)[C@H]3C)C2(CC2)C1] | B194 |
| 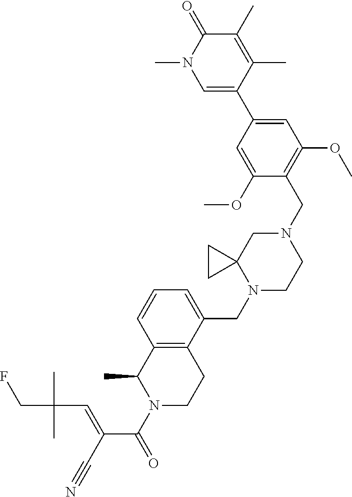 [SMILES: COc1cc(-c2cn(C)c(=O)c(C)c2C)cc(OC)c1CN1CCN(Cc2cccc3c2CCN(C(=O)/C(C#N)=C/C(C)(C)CF)[C@H]3C)C2(CC2)C1] | B195 |
| 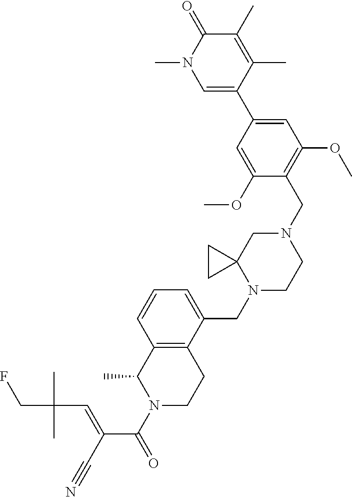 [SMILES: COc1cc(-c2cn(C)c(=O)c(C)c2C)cc(OC)c1CN1CCN(Cc2cccc3c2CCN(C(=O)/C(C#N)=C/C(C)(C)CF)[C@@H]3C)C2(CC2)C1] | B196 |
| 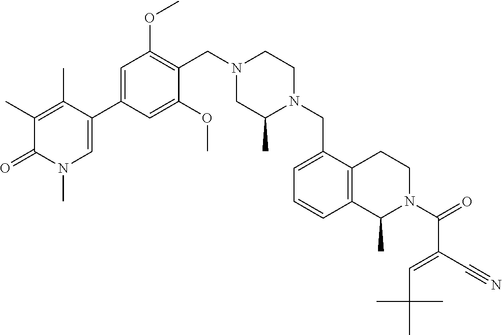 [SMILES: COc1cc(-c2cn(C)c(=O)c(C)c2C)cc(OC)c1CN1CCN(Cc2cccc3c2CCN(C(=O)/C(C#N)=C/C(C)(C)C)[C@H]3C)[C@@H](C)C1] | B197 |
| 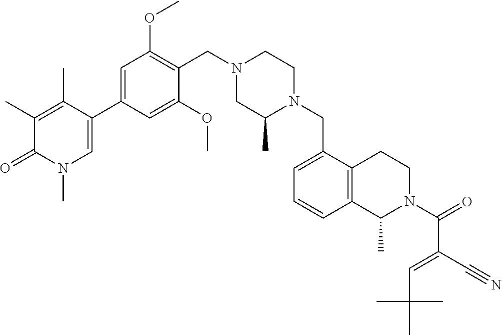 [SMILES: COc1cc(-c2cn(C)c(=O)c(C)c2C)cc(OC)c1CN1CCN(Cc2cccc3c2CCN(C(=O)/C(C#N)=C/C(C)(C)C)[C@@H]3C)[C@@H](C)C1] | B198 |
| 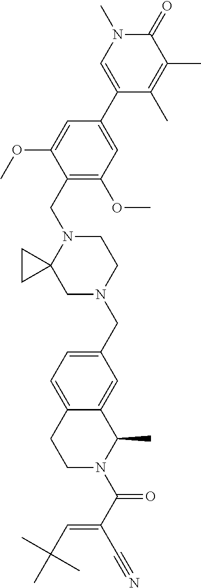 | B199 |
| 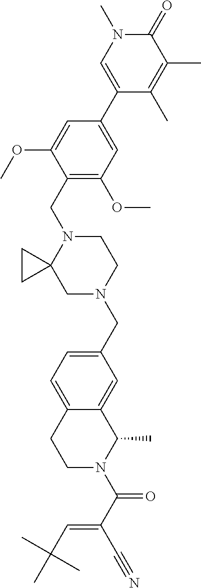 | B200 |
| 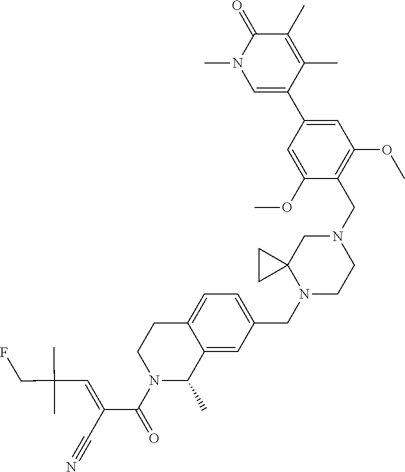 [SMILES: COc1cc(-c2cn(C)c(=O)c(C)c2C)cc(OC)c1CN1CCN(Cc2ccc3c(c2)[C@H](C)N(C(=O)/C(C#N)=C/C(C)(C)CF)CC3)C2(CC2)C1] | B201 |
| 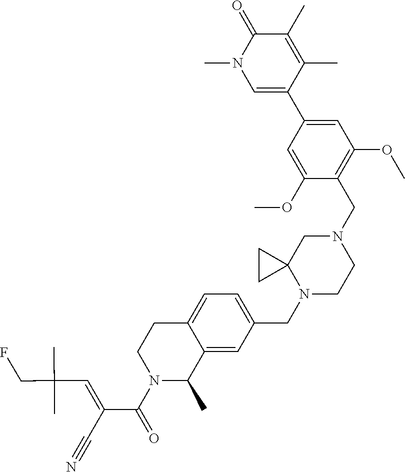 [SMILES: COc1cc(-c2cn(C)c(=O)c(C)c2C)cc(OC)c1CN1CCN(Cc2ccc3c(c2)[C@@H](C)N(C(=O)/C(C#N)=C/C(C)(C)CF)CC3)C2(CC2)C1] | B202 |
| 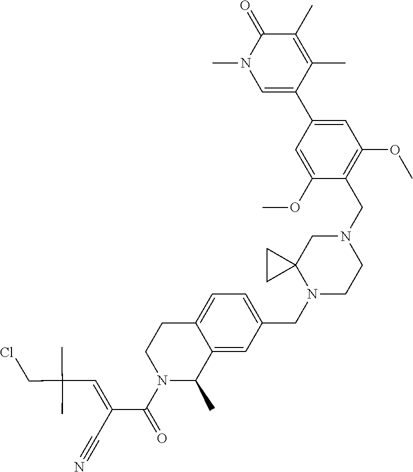 [SMILES: COc1cc(-c2cn(C)c(=O)c(C)c2C)cc(OC)c1CN1CCN(Cc2ccc3c(c2)[C@@H](C)N(C(=O)/C(C#N)=C/C(C)(C)CCl)CC3)C2(CC2)C1] | B203 |
| 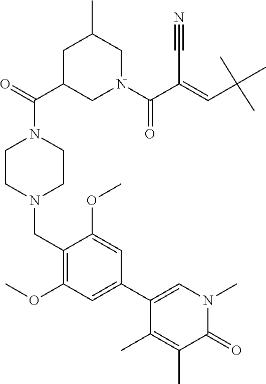 [SMILES: COc1cc(-c2cn(C)c(=O)c(C)c2C)cc(OC)c1CN1CCN(C(=O)C2CC(C)CN(C(=O)/C(C#N)=C/C(C)(C)C)C2)CC1] | B204 |
| 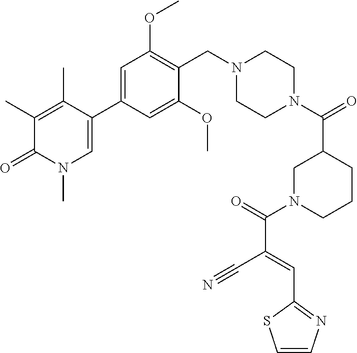 [SMILES: COc1cc(-c2cn(C)c(=O)c(C)c2C)cc(OC)c1CN1CCN(C(=O)C2CCCN(C(=O)/C(C#N)=C/c3nccs3)C2)CC1] | B205 |
| 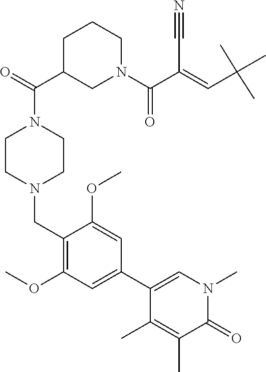 [SMILES: COc1cc(-c2cn(C)c(=O)c(C)c2C)cc(OC)c1CN1CCN(C(=O)C2CCCN(C(=O)/C(C#N)=C/C(C)(C)C)C2)CC1] | B206 |
| 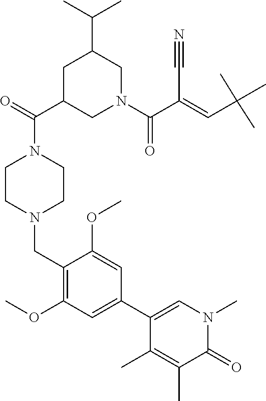 [SMILES: COc1cc(-c2cn(C)c(=O)c(C)c2C)cc(OC)c1CN1CCN(C(=O)C2CC(C(C)C)CN(C(=O)/C(C#N)=C/C(C)(C)C)C2)CC1] | B207 |
| 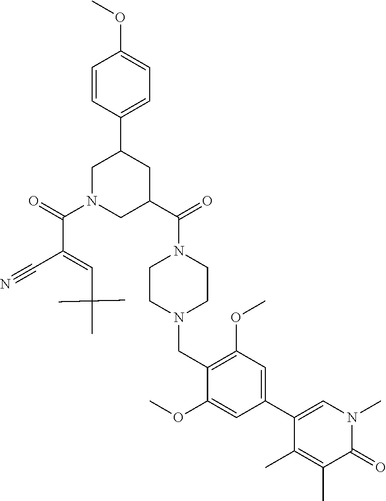 [SMILES: COc1ccc(C2CC(C(=O)N3CCN(Cc4c(OC)cc(-c5cn(C)c(=O)c(C)c5C)cc4OC)CC3)CN(C(=O)/C(C#N)=C/C(C)(C)C)C2)cc1] | B208 |
| 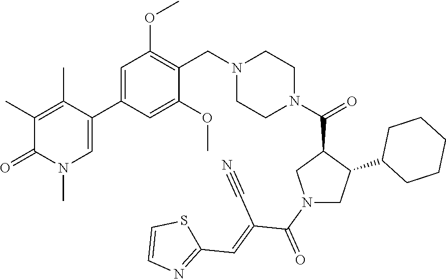 [SMILES: COc1cc(-c2cn(C)c(=O)c(C)c2C)cc(OC)c1CN1CCN(C(=O)[C@@H]2CN(C(=O)/C(C#N)=C/c3nccs3)C[C@H]2C2CCCCC2)CC1] | B209 |
| 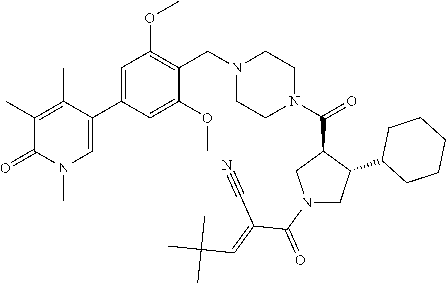 [SMILES: COc1cc(-c2cn(C)c(=O)c(C)c2C)cc(OC)c1CN1CCN(C(=O)[C@@H]2CN(C(=O)/C(C#N)=C/C(C)(C)C)C[C@H]2C2CCCCC2)CC1] | B210 |
| 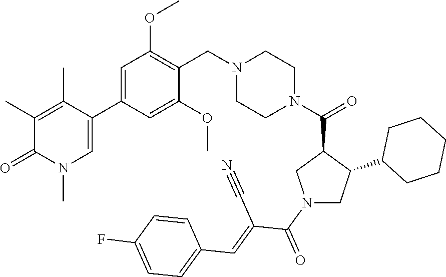 [SMILES: COc1cc(-c2cn(C)c(=O)c(C)c2C)cc(OC)c1CN1CCN(C(=O)[C@@H]2CN(C(=O)/C(C#N)=C/c3ccc(F)cc3)C[C@H]2C2CCCCC2)CC1] | B211 |
| 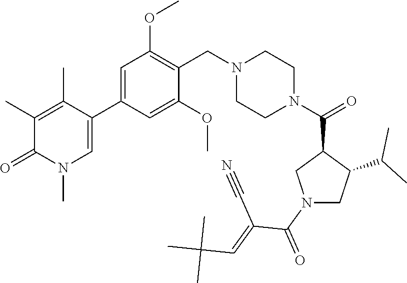 [SMILES: COc1cc(-c2cn(C)c(=O)c(C)c2C)cc(OC)c1CN1CCN(C(=O)[C@@H]2CN(C(=O)/C(C#N)=C/C(C)(C)C)C[C@H]2C(C)C)CC1] | B212 |
| 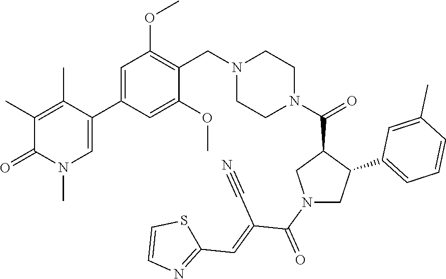 [SMILES: COc1cc(-c2cn(C)c(=O)c(C)c2C)cc(OC)c1CN1CCN(C(=O)[C@@H]2CN(C(=O)/C(C#N)=C/c3nccs3)C[C@H]2c2cccc(C)c2)CC1] | B213 |
| 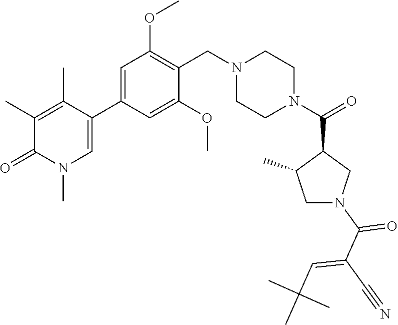 [SMILES: COc1cc(-c2cn(C)c(=O)c(C)c2C)cc(OC)c1CN1CCN(C(=O)[C@H]2CN(C(=O)/C(C#N)=C/C(C)(C)C)C[C@@H]2C)CC1] | B214 |
| 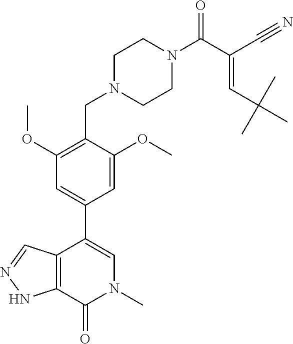 [SMILES: COc1cc(-c2cn(C)c(=O)c3[nH]ncc23)cc(OC)c1CN1CCN(C(=O)/C(C#N)=C/C(C)(C)C)CC1] | B215 |
| 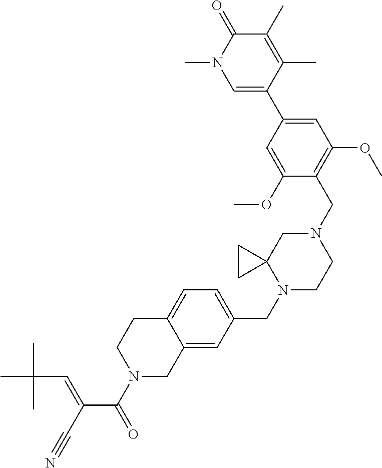 [SMILES: COc1cc(-c2cn(C)c(=O)c(C)c2C)cc(OC)c1CN1CCN(Cc2ccc3c(c2)CN(C(=O)/C(C#N)=C/C(C)(C)C)CC3)C2(CC2)C1] | C1 |
| 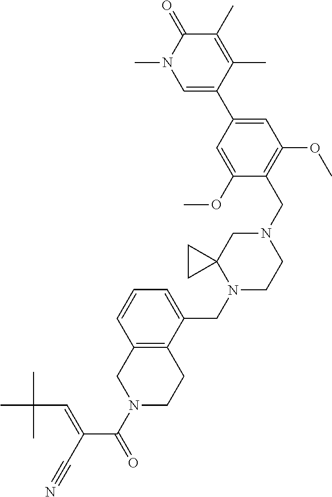 [SMILES: COc1cc(-c2cn(C)c(=O)c(C)c2C)cc(OC)c1CN1CCN(Cc2cccc3c2CCN(C(=O)/C(C#N)=C/C(C)(C)C)C3)C2(CC2)C1] | C2 |
| 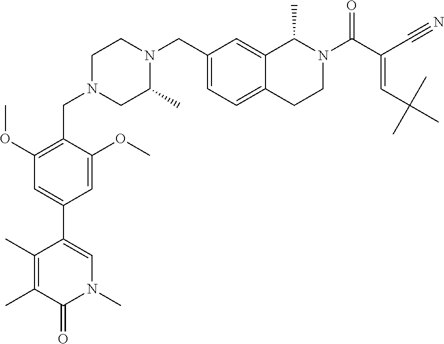 [SMILES: COc1cc(-c2cn(C)c(=O)c(C)c2C)cc(OC)c1CN1CCN(Cc2ccc3c(c2)[C@H](C)N(C(=O)/C(C#N)=C/C(C)(C)C)CC3)[C@H](C)C1] | C3 |
| 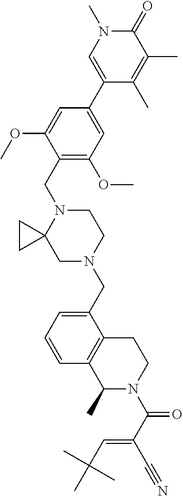 | C4 |
| 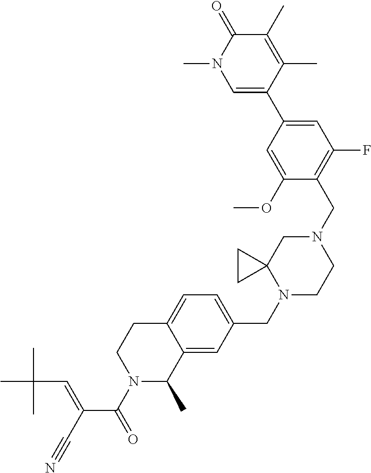 [SMILES: COc1cc(-c2cn(C)c(=O)c(C)c2C)cc(F)c1CN1CCN(Cc2ccc3c(c2)[C@@H](C)N(C(=O)/C(C#N)=C/C(C)(C)C)CC3)C2(CC2)C1] | C5 |
| 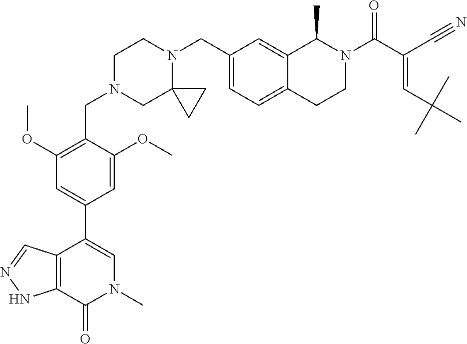 [SMILES: COc1cc(-c2cn(C)c(=O)c3[nH]ncc23)cc(OC)c1CN1CCN(Cc2ccc3c(c2)[C@@H](C)N(C(=O)/C(C#N)=C/C(C)(C)C)CC3)C2(CC2)C1] | C6 |
| 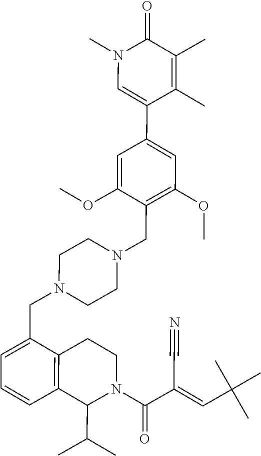 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C(C)C)CC1] | C7 |
| 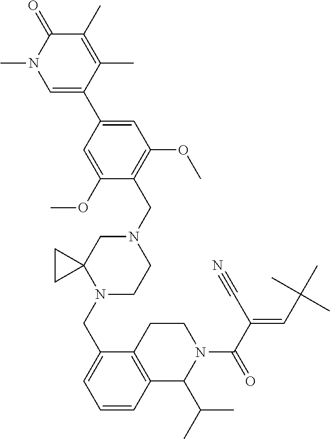 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C(C)C)C2(CC2)C1] | C8 |
| 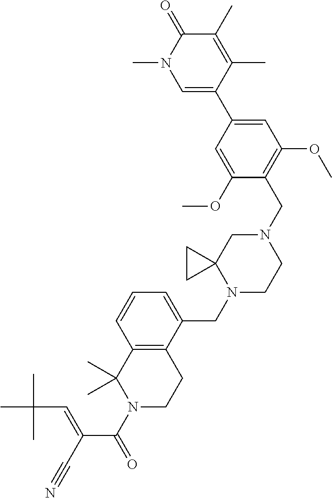 [SMILES: COc1cc(-c2cn(C)c(=O)c(C)c2C)cc(OC)c1CN1CCN(Cc2cccc3c2CCN(C(=O)/C(C#N)=C/C(C)(C)C)C3(C)C)C2(CC2)C1] | C9 |
| 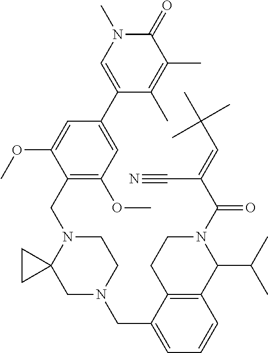 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C(C)C)CC12CC2] | C10 |
| 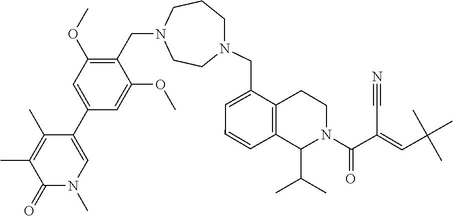 [SMILES: COc1cc(-c2cn(C)c(=O)c(C)c2C)cc(OC)c1CN1CCCN(Cc2cccc3c2CCN(C(=O)/C(C#N)=C/C(C)(C)C)C3C(C)C)CC1] | C11 |
| 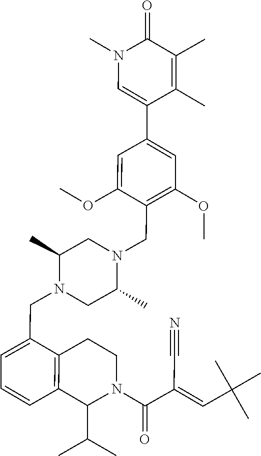 [SMILES: COc1cc(-c2cn(C)c(=O)c(C)c2C)cc(OC)c1CN1C[C@H](C)N(Cc2cccc3c2CCN(C(=O)/C(C#N)=C/C(C)(C)C)C3C(C)C)C[C@H]1C] | C12 |
| 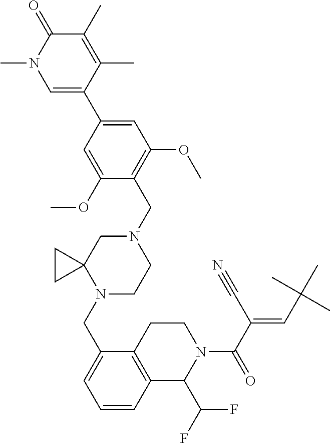 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C(F)F)C2(CC2)C1] | C13 |
| 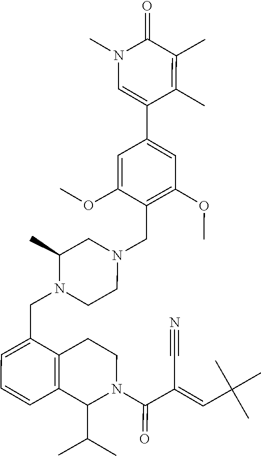 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C(C)C)[C@@H](C)C1] | C14 |
| 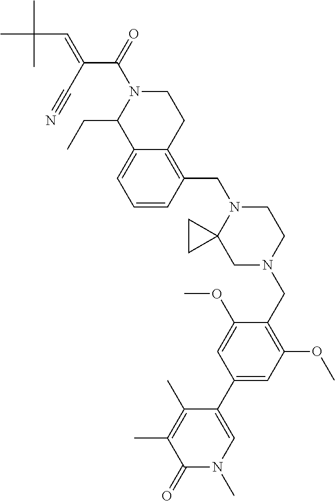 [SMILES: CCC1c2cccc(CN3CCN(Cc4c(OC)cc(-c5cn(C)c(=O)c(C)c5C)cc4OC)CC34CC4)c2CCN1C(=O)/C(C#N)=C/C(C)(C)C] | C15 |
| 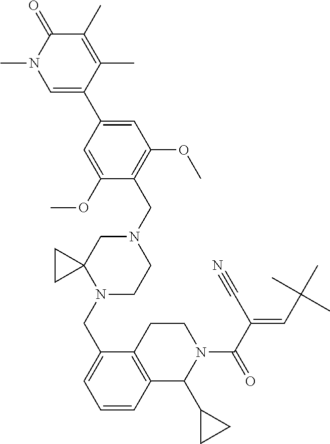 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C2CC2)C2(CC2)C1] | C16 |
| 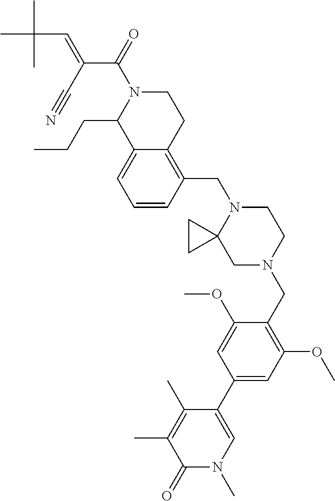 [SMILES: CCCC1c2cccc(CN3CCN(Cc4c(OC)cc(-c5cn(C)c(=O)c(C)c5C)cc4OC)CC34CC4)c2CCN1C(=O)/C(C#N)=C/C(C)(C)C] | C17 |
| 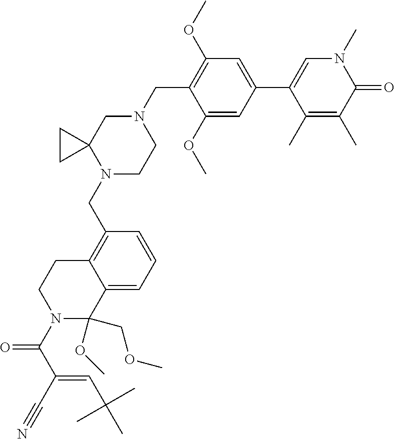 [SMILES: COCC1(OC)c2cccc(CN3CCN(Cc4c(OC)cc(-c5cn(C)c(=O)c(C)c5C)cc4OC)CC34CC4)c2CCN1C(=O)/C(C#N)=C/C(C)(C)C] | C18 |
| 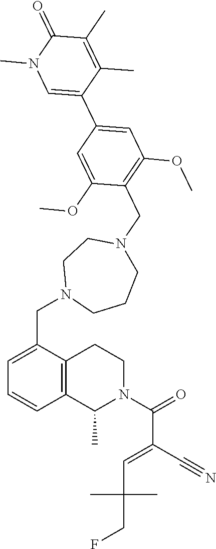 [SMILES: COc1cc(-c2cn(C)c(=O)c(C)c2C)cc(OC)c1CN1CCCN(Cc2cccc3c2CCN(C(=O)/C(C#N)=C/C(C)(C)CF)[C@@H]3C)CC1] | C19 |
| 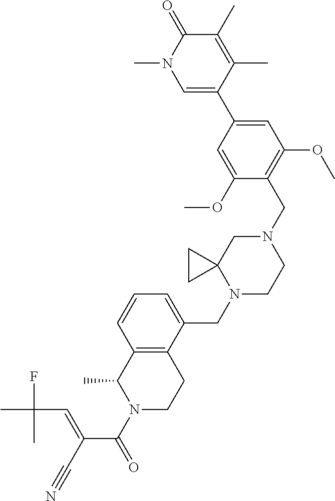 [SMILES: COc1cc(-c2cn(C)c(=O)c(C)c2C)cc(OC)c1CN1CCN(Cc2cccc3c2CCN(C(=O)/C(C#N)=C/C(C)(C)F)[C@@H]3C)C2(CC2)C1] | C20 |
| 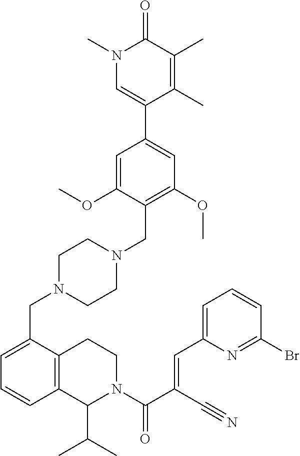 [SMILES: COc1cc(-c2cn(C)c(=O)c(C)c2C)cc(OC)c1CN1CCN(Cc2cccc3c2CCN(C(=O)/C(C#N)=C/c2cccc(Br)n2)C3C(C)C)CC1] | C21 |
| 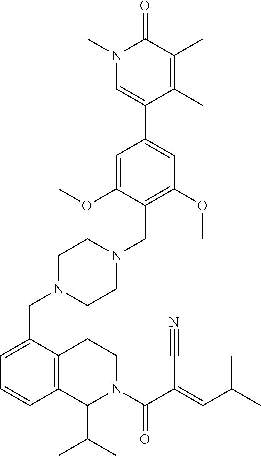 [SMILES: COc1cc(-c2cn(C)c(=O)c(C)c2C)cc(OC)c1CN1CCN(Cc2cccc3c2CCN(C(=O)/C(C#N)=C/C(C)C)C3C(C)C)CC1] | C22 |
| 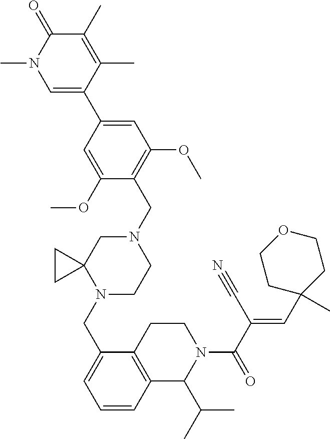 | C23 |
| 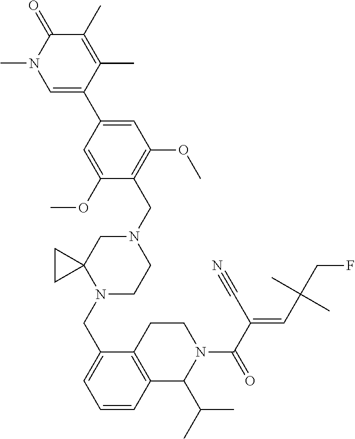 [SMILES: COc1cc(-c2cn(C)c(=O)c(C)c2C)cc(OC)c1CN1CCN(Cc2cccc3c2CCN(C(=O)/C(C#N)=C/C(C)(C)CF)C3C(C)C)C2(CC2)C1] | C24 |
| 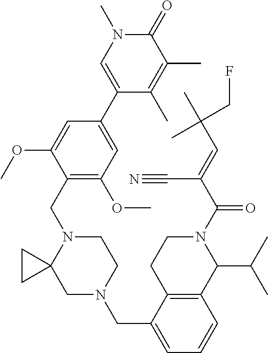 [SMILES: COc1cc(-c2cn(C)c(=O)c(C)c2C)cc(OC)c1CN1CCN(Cc2cccc3c2CCN(C(=O)/C(C#N)=C/C(C)(C)CF)C3C(C)C)CC12CC2] | C25 |
| 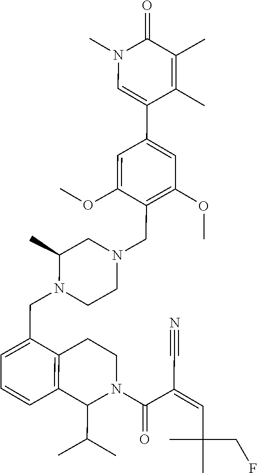 [SMILES: COc1cc(-c2cn(C)c(=O)c(C)c2C)cc(OC)c1CN1CCN(Cc2cccc3c2CCN(C(=O)/C(C#N)=C\C(C)(C)CF)C3C(C)C)[C@@H](C)C1] | C26 |
| 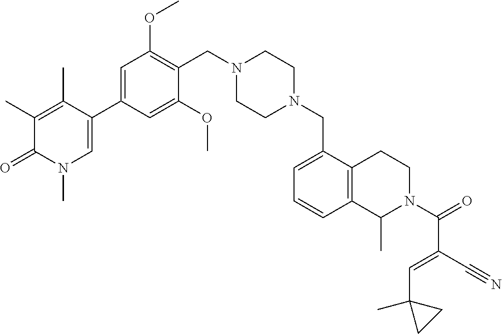 [SMILES: COc1cc(-c2cn(C)c(=O)c(C)c2C)cc(OC)c1CN1CCN(Cc2cccc3c2CCN(C(=O)/C(C#N)=C/C2(C)CC2)C3C)CC1] | C27 |
| 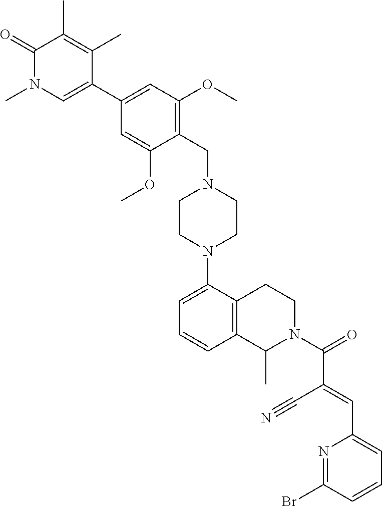 [SMILES: COc1cc(-c2cn(C)c(=O)c(C)c2C)cc(OC)c1CN1CCN(c2cccc3c2CCN(C(=O)/C(C#N)=C/c2cccc(Br)n2)C3C)CC1] | C28 |
| 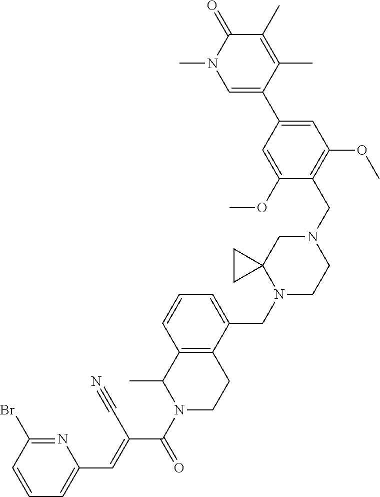 [SMILES: COc1cc(-c2cn(C)c(=O)c(C)c2C)cc(OC)c1CN1CCN(Cc2cccc3c2CCN(C(=O)/C(C#N)=C/c2cccc(Br)n2)C3C)C2(CC2)C1] | C29 |
| 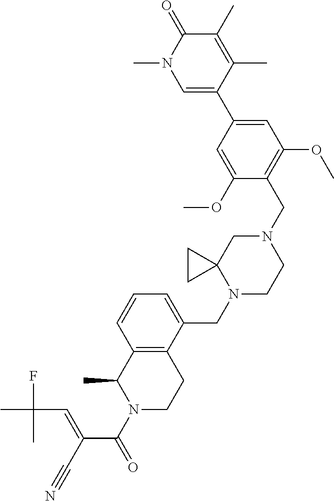 [SMILES: COc1cc(-c2cn(C)c(=O)c(C)c2C)cc(OC)c1CN1CCN(Cc2cccc3c2CCN(C(=O)/C(C#N)=C/C(C)(C)F)[C@H]3C)C2(CC2)C1] | C30 |
| 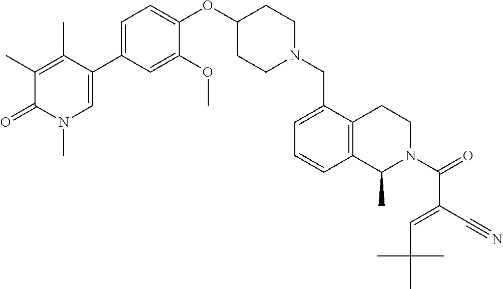 [SMILES: COc1cc(-c2cn(C)c(=O)c(C)c2C)ccc1OC1CCN(Cc2cccc3c2CCN(C(=O)/C(C#N)=C/C(C)(C)C)[C@H]3C)CC1] | C31 |
| 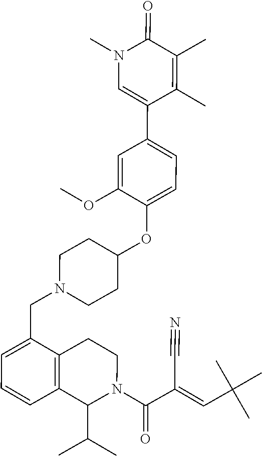 [SMILES: COc1cc(-c2cn(C)c(=O)c(C)c2C)ccc1OC1CCN(Cc2cccc3c2CCN(C(=O)/C(C#N)=C/C(C)(C)C)C3C(C)C)CC1] | C32 |
| 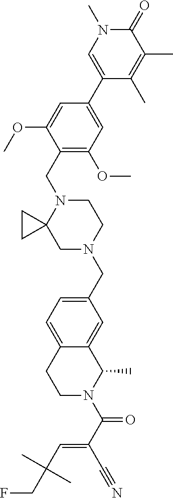 | C33 |
| 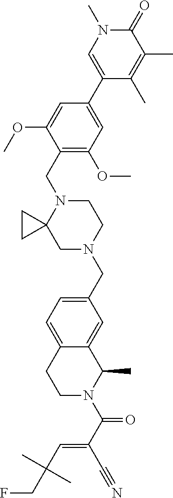 | C34 |
| 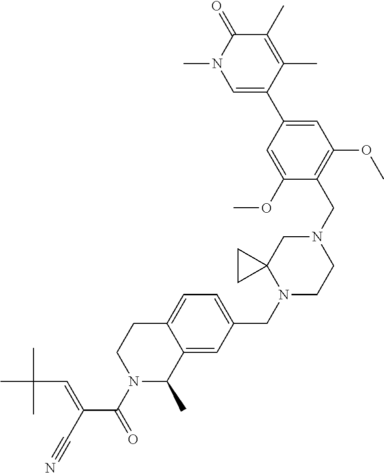 [SMILES: COc1cc(-c2cn(C)c(=O)c(C)c2C)cc(OC)c1CN1CCN(Cc2ccc3c(c2)[C@@H](C)N(C(=O)/C(C#N)=C/C(C)(C)C)CC3)C2(CC2)C1] | C35 |
| 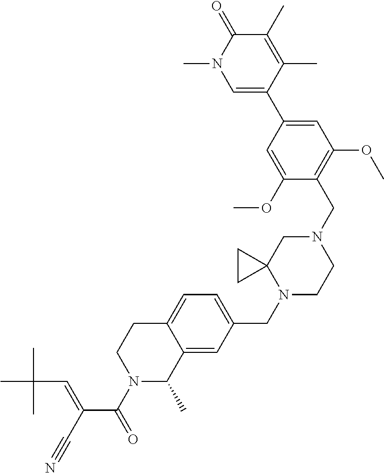 [SMILES: COc1cc(-c2cn(C)c(=O)c(C)c2C)cc(OC)c1CN1CCN(Cc2ccc3c(c2)[C@H](C)N(C(=O)/C(C#N)=C/C(C)(C)C)CC3)C2(CC2)C1] | C36 |
| 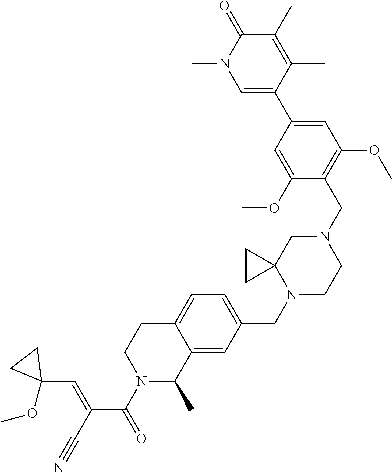 | C37 |
| 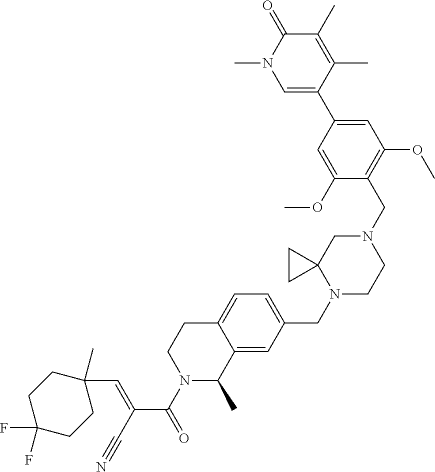 [SMILES: COc1cc(-c2cn(C)c(=O)c(C)c2C)cc(OC)c1CN1CCN(Cc2ccc3c(c2)[C@@H](C)N(C(=O)/C(C#N)=C/C2(C)CCC(F)(F)CC2)CC3)C2(CC2)C1] | C38 |
| 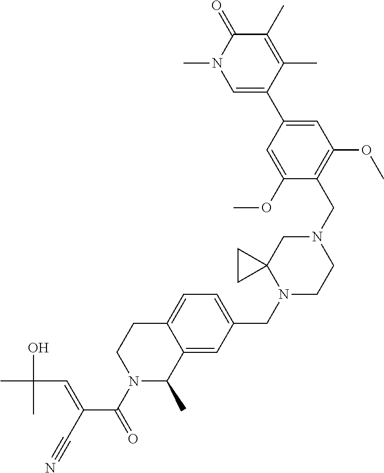 [SMILES: COc1cc(-c2cn(C)c(=O)c(C)c2C)cc(OC)c1CN1CCN(Cc2ccc3c(c2)[C@@H](C)N(C(=O)/C(C#N)=C/C(C)(C)O)CC3)C2(CC2)C1] | C39 |
| 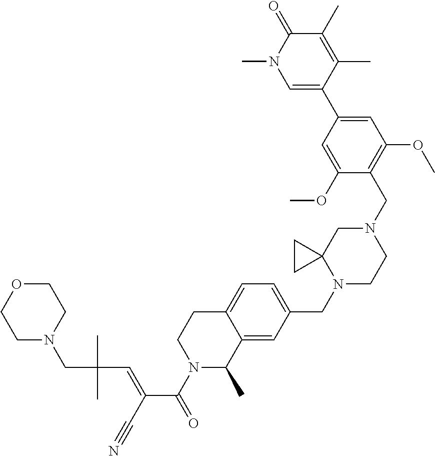 [SMILES: COc1cc(-c2cn(C)c(=O)c(C)c2C)cc(OC)c1CN1CCN(Cc2ccc3c(c2)[C@@H](C)N(C(=O)/C(C#N)=C/C(C)(C)CN2CCOCC2)CC3)C2(CC2)C1] | C40 |
| 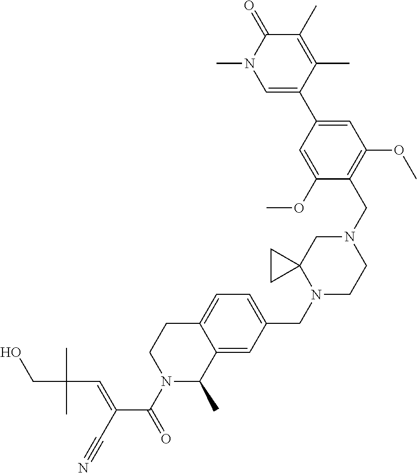 [SMILES: COc1cc(-c2cn(C)c(=O)c(C)c2C)cc(OC)c1CN1CCN(Cc2ccc3c(c2)[C@@H](C)N(C(=O)/C(C#N)=C/C(C)(C)CO)CC3)C2(CC2)C1] | C41 |
| 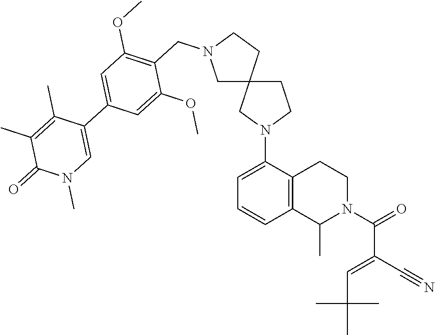 [SMILES: COc1cc(-c2cn(C)c(=O)c(C)c2C)cc(OC)c1CN1CCC2(CCN(c3cccc4c3CCN(C(=O)/C(C#N)=C/C(C)(C)C)C4C)C2)C1] | C42 |
| 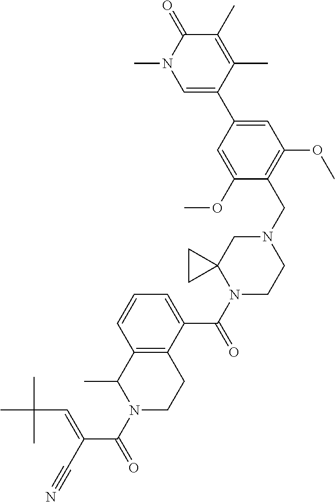 [SMILES: COc1cc(-c2cn(C)c(=O)c(C)c2C)cc(OC)c1CN1CCN(C(=O)c2cccc3c2CCN(C(=O)/C(C#N)=C/C(C)(C)C)C3C)C2(CC2)C1] | C43 |
| 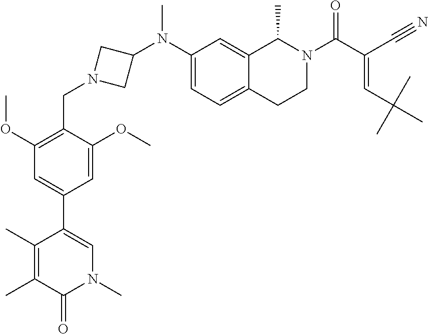 [SMILES: COc1cc(-c2cn(C)c(=O)c(C)c2C)cc(OC)c1CN1CC(N(C)c2ccc3c(c2)[C@H](C)N(C(=O)/C(C#N)=C/C(C)(C)C)CC3)C1] | C44 |
| 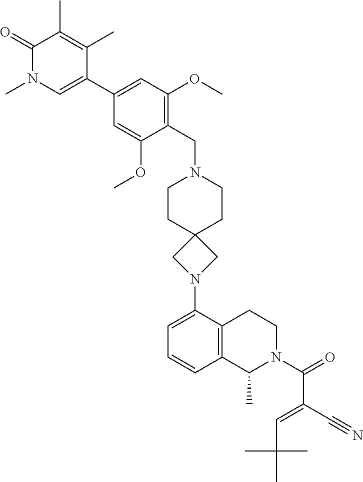 [SMILES: COc1cc(-c2cn(C)c(=O)c(C)c2C)cc(OC)c1CN1CCC2(CC1)CN(c1cccc3c1CCN(C(=O)/C(C#N)=C/C(C)(C)C)[C@@H]3C)C2] | C45 |
| 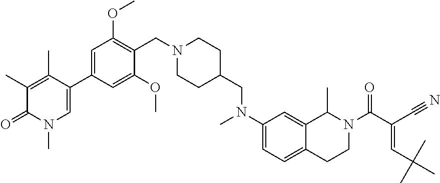 [SMILES: COc1cc(-c2cn(C)c(=O)c(C)c2C)cc(OC)c1CN1CCC(CN(C)c2ccc3c(c2)C(C)N(C(=O)/C(C#N)=C/C(C)(C)C)CC3)CC1] | C46 |
| 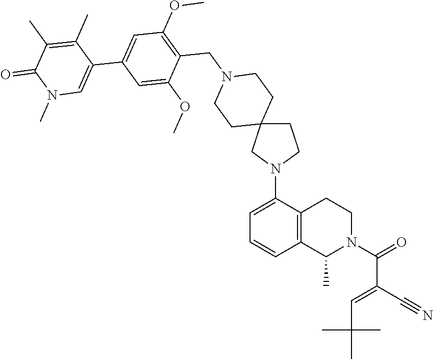 [SMILES: COc1cc(-c2cn(C)c(=O)c(C)c2C)cc(OC)c1CN1CCC2(CC1)CCN(c1cccc3c1CCN(C(=O)/C(C#N)=C/C(C)(C)C)[C@@H]3C)C2] | C47 |
| 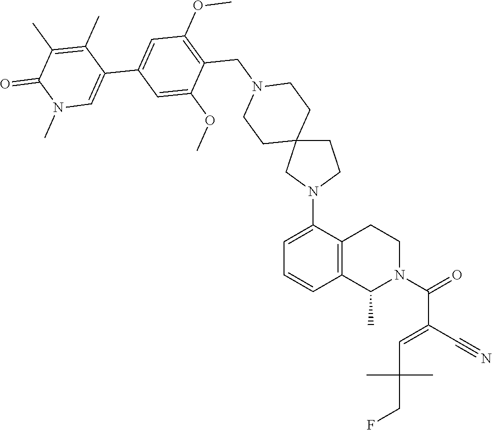 [SMILES: COc1cc(-c2cn(C)c(=O)c(C)c2C)cc(OC)c1CN1CCC2(CC1)CCN(c1cccc3c1CCN(C(=O)/C(C#N)=C/C(C)(C)CF)[C@@H]3C)C2] | C48 |
| 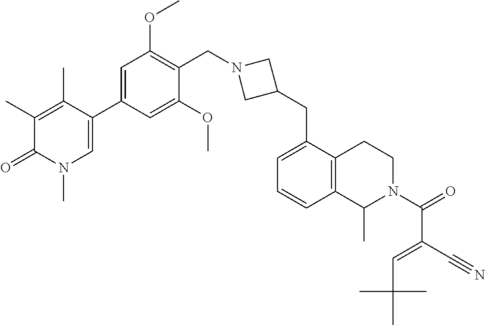 [SMILES: COc1cc(-c2cn(C)c(=O)c(C)c2C)cc(OC)c1CN1CC(Cc2cccc3c2CCN(C(=O)/C(C#N)=C/C(C)(C)C)C3C)C1] | C49 |
| 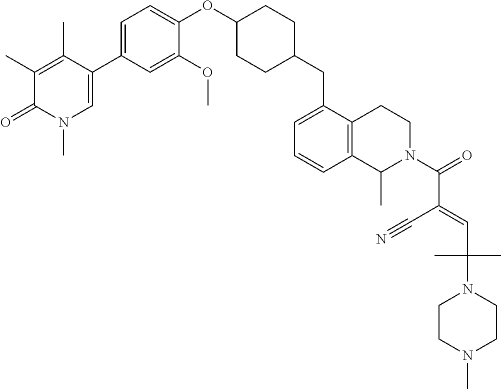 [SMILES: COc1cc(-c2cn(C)c(=O)c(C)c2C)ccc1OC1CCC(Cc2cccc3c2CCN(C(=O)/C(C#N)=C/C(C)(C)N2CCN(C)CC2)C3C)CC1] | C50 |
| 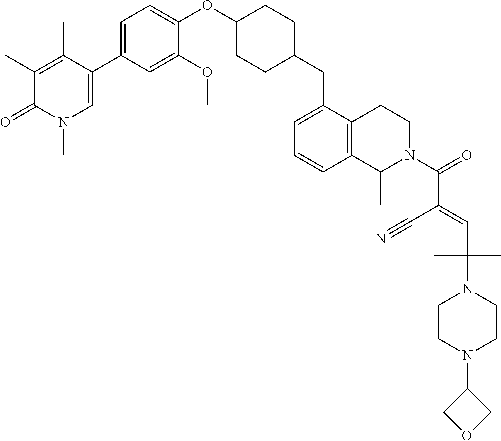 [SMILES: COc1cc(-c2cn(C)c(=O)c(C)c2C)ccc1OC1CCC(Cc2cccc3c2CCN(C(=O)/C(C#N)=C/C(C)(C)N2CCN(C4COC4)CC2)C3C)CC1] | C51 |
| 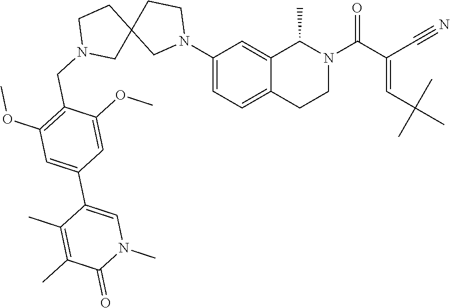 [SMILES: COc1cc(-c2cn(C)c(=O)c(C)c2C)cc(OC)c1CN1CCC2(CCN(c3ccc4c(c3)[C@H](C)N(C(=O)/C(C#N)=C/C(C)(C)C)CC4)C2)C1] | C52 |
| 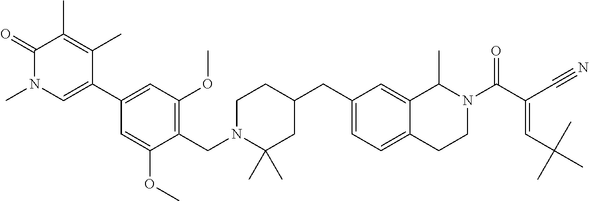 [SMILES: COc1cc(-c2cn(C)c(=O)c(C)c2C)cc(OC)c1CN1CCC(Cc2ccc3c(c2)C(C)N(C(=O)/C(C#N)=C/C(C)(C)C)CC3)CC1(C)C] | C53 |
| 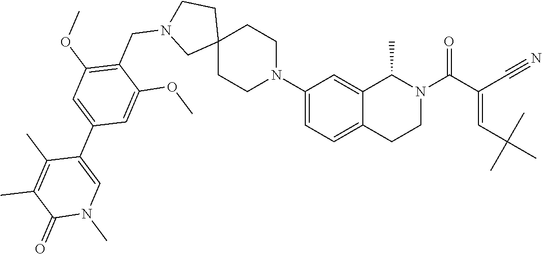 [SMILES: COc1cc(-c2cn(C)c(=O)c(C)c2C)cc(OC)c1CN1CCC2(CCN(c3ccc4c(c3)[C@H](C)N(C(=O)/C(C#N)=C/C(C)(C)C)CC4)CC2)C1] | C54 |
| 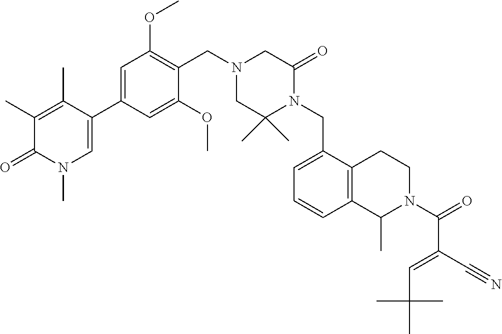 [SMILES: COc1cc(-c2cn(C)c(=O)c(C)c2C)cc(OC)c1CN1CC(=O)N(Cc2cccc3c2CCN(C(=O)/C(C#N)=C/C(C)(C)C)C3C)C(C)(C)C1] | C55 |
| 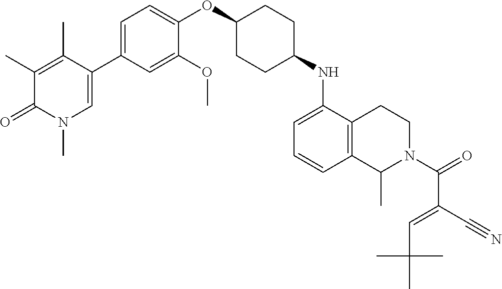 [SMILES: COc1cc(-c2cn(C)c(=O)c(C)c2C)ccc1O[C@H]1CC[C@@H](Nc2cccc3c2CCN(C(=O)/C(C#N)=C/C(C)(C)C)C3C)CC1] | C56 |
| 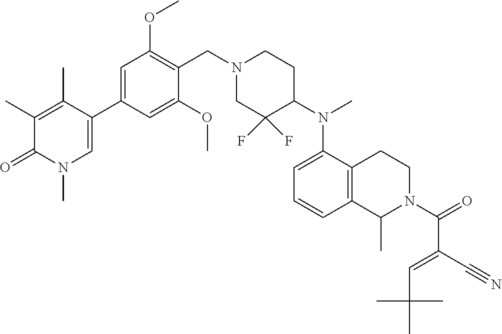 [SMILES: COc1cc(-c2cn(C)c(=O)c(C)c2C)cc(OC)c1CN1CCC(N(C)c2cccc3c2CCN(C(=O)/C(C#N)=C/C(C)(C)C)C3C)C(F)(F)C1] | C57 |
| 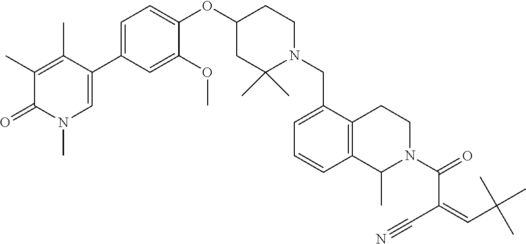 [SMILES: COc1cc(-c2cn(C)c(=O)c(C)c2C)ccc1OC1CCN(Cc2cccc3c2CCN(C(=O)/C(C#N)=C\C(C)(C)C)C3C)C(C)(C)C1] | C58 |
| 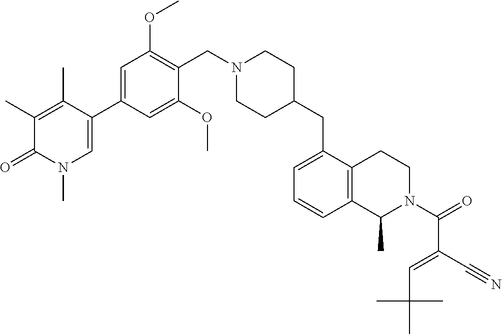 [SMILES: COc1cc(-c2cn(C)c(=O)c(C)c2C)cc(OC)c1CN1CCC(Cc2cccc3c2CCN(C(=O)/C(C#N)=C/C(C)(C)C)[C@H]3C)CC1] | C59 |
| 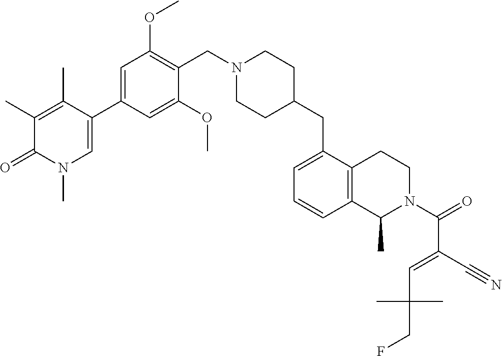 [SMILES: COc1cc(-c2cn(C)c(=O)c(C)c2C)cc(OC)c1CN1CCC(Cc2cccc3c2CCN(C(=O)/C(C#N)=C/C(C)(C)CF)[C@H]3C)CC1] | C60 |
| 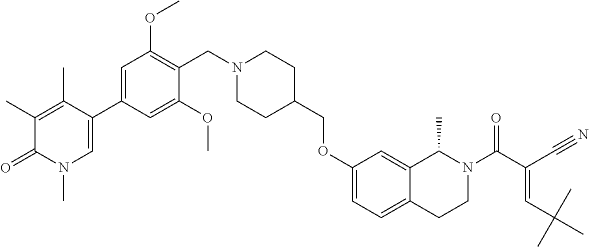 [SMILES: COc1cc(-c2cn(C)c(=O)c(C)c2C)cc(OC)c1CN1CCC(COc2ccc3c(c2)[C@H](C)N(C(=O)/C(C#N)=C/C(C)(C)C)CC3)CC1] | C61 |
| 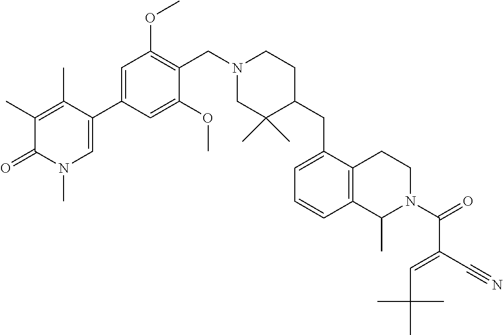 [SMILES: COc1cc(-c2cn(C)c(=O)c(C)c2C)cc(OC)c1CN1CCC(Cc2cccc3c2CCN(C(=O)/C(C#N)=C/C(C)(C)C)C3C)C(C)(C)C1] | C62 |
| 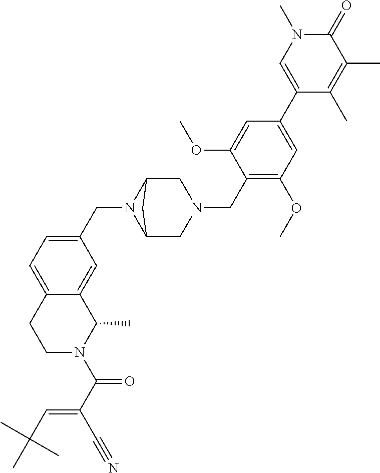 [SMILES: COc1cc(-c2cn(C)c(=O)c(C)c2C)cc(OC)c1CN1CC2CC(C1)N2Cc1ccc2c(c1)[C@H](C)N(C(=O)/C(C#N)=C/C(C)(C)C)CC2] | C63 |
| 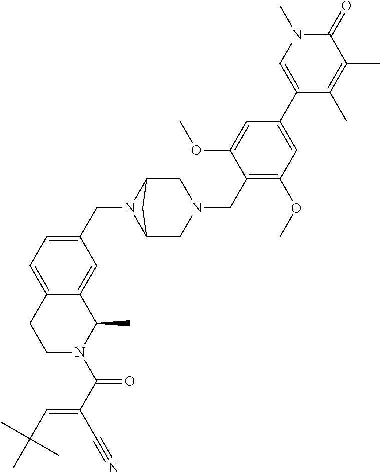 [SMILES: COc1cc(-c2cn(C)c(=O)c(C)c2C)cc(OC)c1CN1CC2CC(C1)N2Cc1ccc2c(c1)[C@@H](C)N(C(=O)/C(C#N)=C/C(C)(C)C)CC2] | C64 |
| 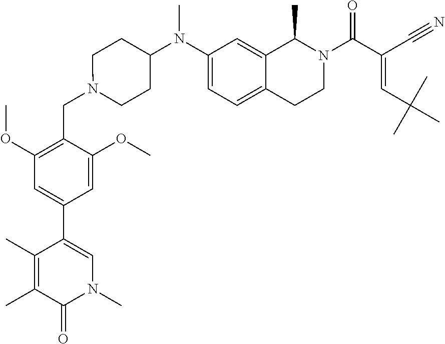 [SMILES: COc1cc(-c2cn(C)c(=O)c(C)c2C)cc(OC)c1CN1CCC(N(C)c2ccc3c(c2)[C@@H](C)N(C(=O)/C(C#N)=C/C(C)(C)C)CC3)CC1] | C65 |
| 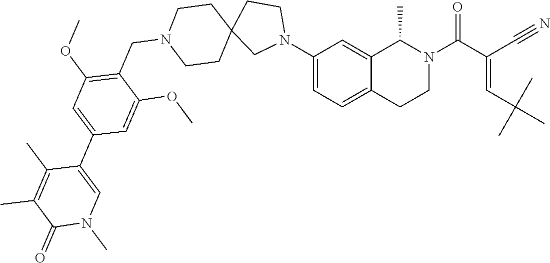 [SMILES: COc1cc(-c2cn(C)c(=O)c(C)c2C)cc(OC)c1CN1CCC2(CC1)CCN(c1ccc3c(c1)[C@H](C)N(C(=O)/C(C#N)=C/C(C)(C)C)CC3)C2] | C66 |
| 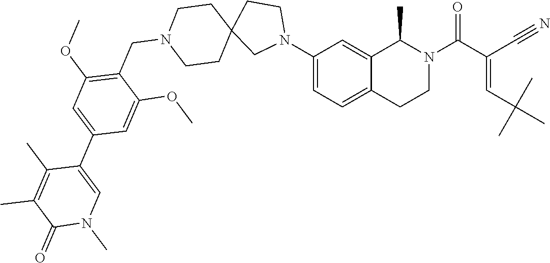 [SMILES: COc1cc(-c2cn(C)c(=O)c(C)c2C)cc(OC)c1CN1CCC2(CC1)CCN(c1ccc3c(c1)[C@@H](C)N(C(=O)/C(C#N)=C/C(C)(C)C)CC3)C2] | C67 |
| 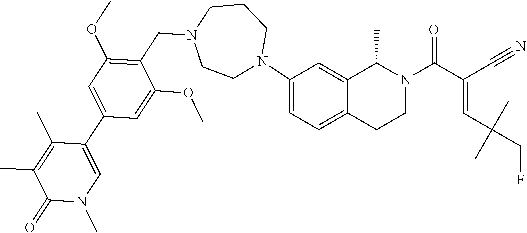 | C68 |
| 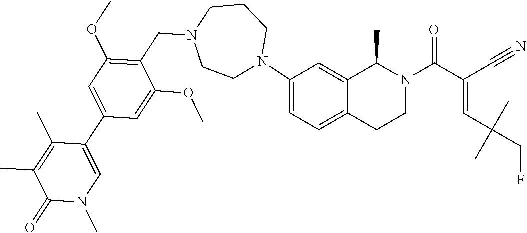 | C69 |
| 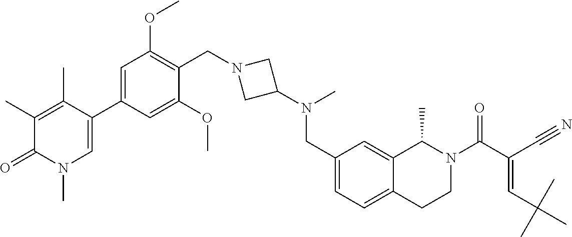 [SMILES: COc1cc(-c2cn(C)c(=O)c(C)c2C)cc(OC)c1CN1CC(N(C)Cc2ccc3c(c2)[C@H](C)N(C(=O)/C(C#N)=C/C(C)(C)C)CC3)C1] | C70 |
| 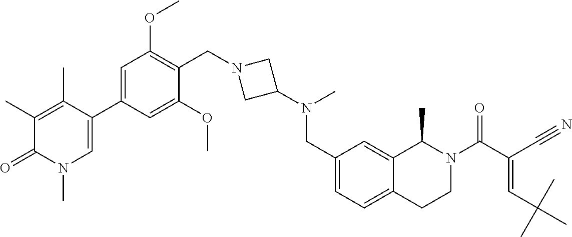 [SMILES: COc1cc(-c2cn(C)c(=O)c(C)c2C)cc(OC)c1CN1CC(N(C)Cc2ccc3c(c2)[C@@H](C)N(C(=O)/C(C#N)=C/C(C)(C)C)CC3)C1] | C71 |
| 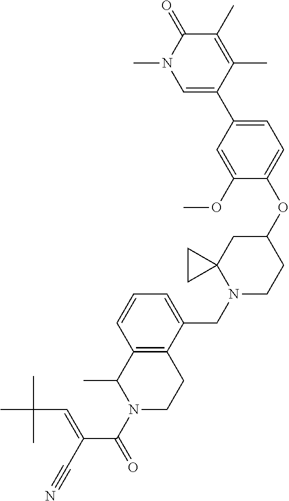 [SMILES: COc1cc(-c2cn(C)c(=O)c(C)c2C)ccc1OC1CCN(Cc2cccc3c2CCN(C(=O)/C(C#N)=C/C(C)(C)C)C3C)C2(CC2)C1] | C72 |
| 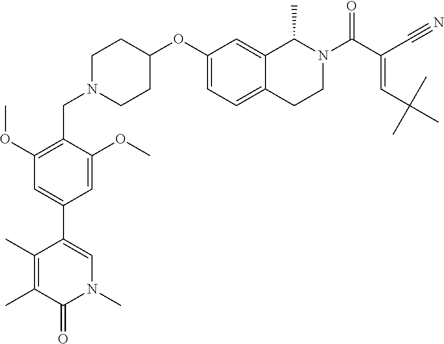 [SMILES: COc1cc(-c2cn(C)c(=O)c(C)c2C)cc(OC)c1CN1CCC(Oc2ccc3c(c2)[C@H](C)N(C(=O)/C(C#N)=C/C(C)(C)C)CC3)CC1] | C73 |
| 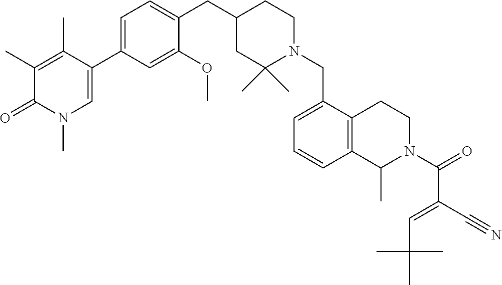 [SMILES: COc1cc(-c2cn(C)c(=O)c(C)c2C)ccc1CC1CCN(Cc2cccc3c2CCN(C(=O)/C(C#N)=C/C(C)(C)C)C3C)C(C)(C)C1] | C74 |
| 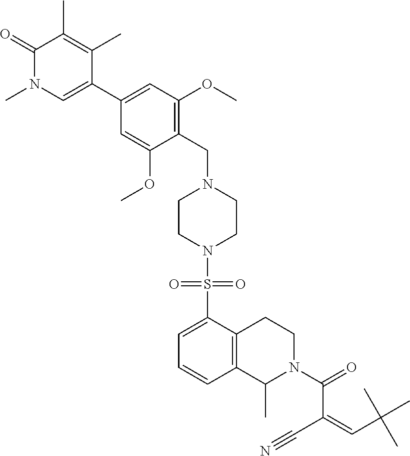 [SMILES: COc1cc(-c2cn(C)c(=O)c(C)c2C)cc(OC)c1CN1CCN(S(=O)(=O)c2cccc3c2CCN(C(=O)/C(C#N)=C\C(C)(C)C)C3C)CC1] | C75 |
| 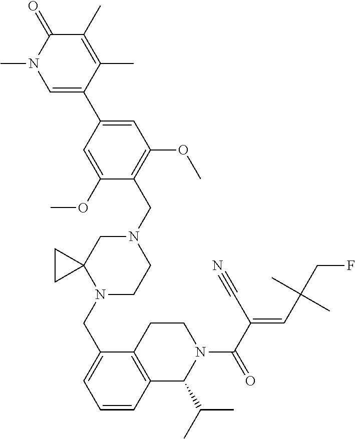 [SMILES: COc1cc(-c2cn(C)c(=O)c(C)c2C)cc(OC)c1CN1CCN(Cc2cccc3c2CCN(C(=O)/C(C#N)=C/C(C)(C)CF)[C@@H]3C(C)C)C2(CC2)C1] | C76 |
| 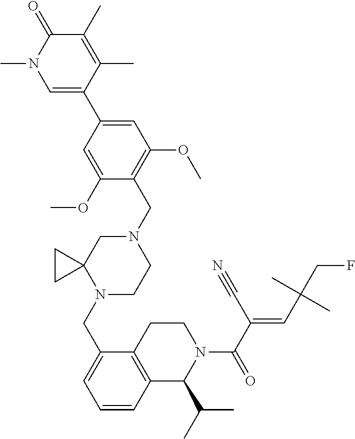 [SMILES: COc1cc(-c2cn(C)c(=O)c(C)c2C)cc(OC)c1CN1CCN(Cc2cccc3c2CCN(C(=O)/C(C#N)=C/C(C)(C)CF)[C@H]3C(C)C)C2(CC2)C1] | C77 |
| 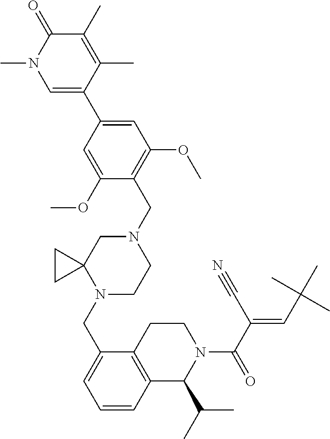 [SMILES: COc1cc(-c2cn(C)c(=O)c(C)c2C)cc(OC)c1CN1CCN(Cc2cccc3c2CCN(C(=O)/C(C#N)=C/C(C)(C)C)[C@H]3C(C)C)C2(CC2)C1] | C78 |
| 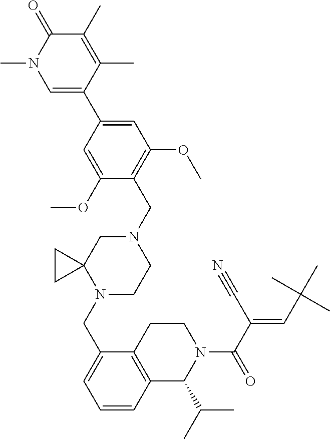 [SMILES: COc1cc(-c2cn(C)c(=O)c(C)c2C)cc(OC)c1CN1CCN(Cc2cccc3c2CCN(C(=O)/C(C#N)=C/C(C)(C)C)[C@@H]3C(C)C)C2(CC2)C1] | C79 |
| 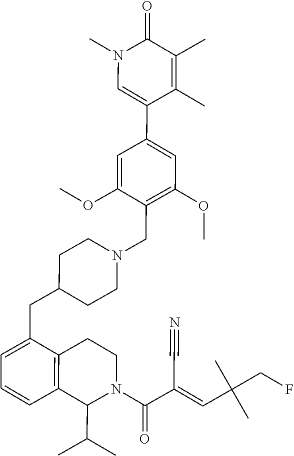 [SMILES: COc1cc(-c2cn(C)c(=O)c(C)c2C)cc(OC)c1CN1CCC(Cc2cccc3c2CCN(C(=O)/C(C#N)=C/C(C)(C)CF)C3C(C)C)CC1] | C80 |
| 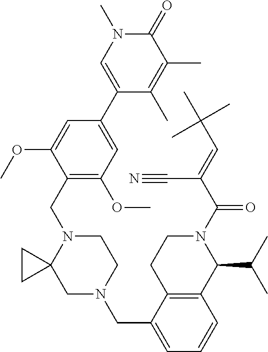 [SMILES: COc1cc(-c2cn(C)c(=O)c(C)c2C)cc(OC)c1CN1CCN(Cc2cccc3c2CCN(C(=O)/C(C#N)=C/C(C)(C)C)[C@H]3C(C)C)CC12CC2] | C81 |
| 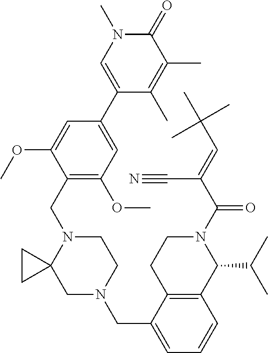 [SMILES: COc1cc(-c2cn(C)c(=O)c(C)c2C)cc(OC)c1CN1CCN(Cc2cccc3c2CCN(C(=O)/C(C#N)=C/C(C)(C)C)[C@@H]3C(C)C)CC12CC2] | C82 |
| 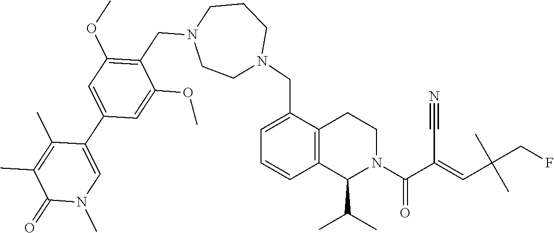 | C83 |
| 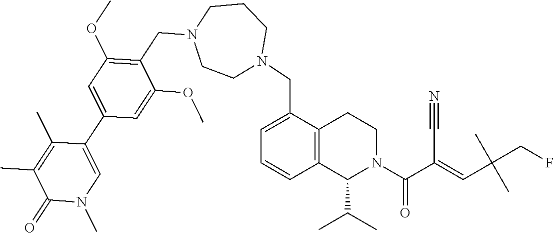 | C84 |
| 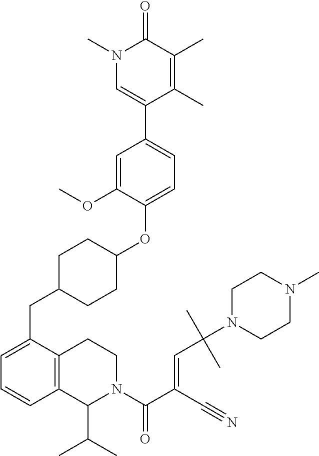 [SMILES: COc1cc(-c2cn(C)c(=O)c(C)c2C)ccc1OC1CCC(Cc2cccc3c2CCN(C(=O)/C(C#N)=C/C(C)(C)N2CCN(C)CC2)C3C(C)C)CC1] | C85 |
| 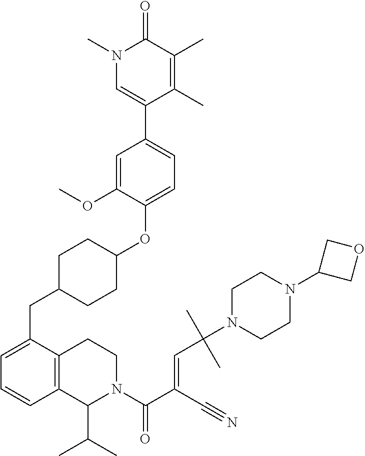 [SMILES: COc1cc(-c2cn(C)c(=O)c(C)c2C)ccc1OC1CCC(Cc2cccc3c2CCN(C(=O)/C(C#N)=C/C(C)(C)N2CCN(C4COC4)CC2)C3C(C)C)CC1] | C86 |
| 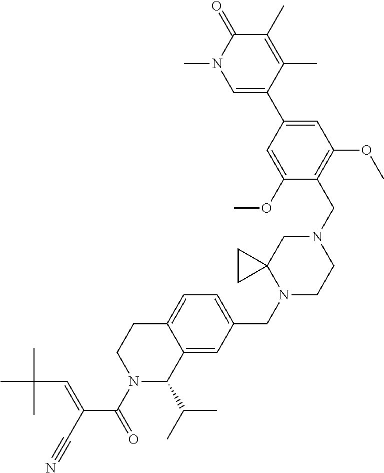 [SMILES: COc1cc(-c2cn(C)c(=O)c(C)c2C)cc(OC)c1CN1CCN(Cc2ccc3c(c2)[C@H](C(C)C)N(C(=O)/C(C#N)=C/C(C)(C)C)CC3)C2(CC2)C1] | C87 |
| 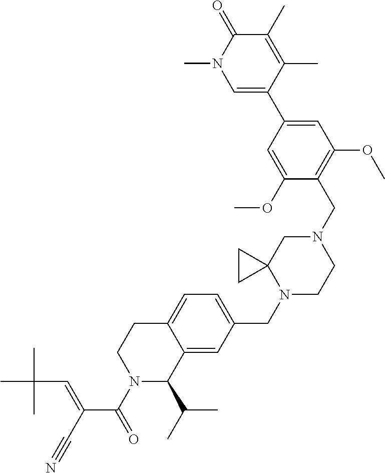 [SMILES: COc1cc(-c2cn(C)c(=O)c(C)c2C)cc(OC)c1CN1CCN(Cc2ccc3c(c2)[C@@H](C(C)C)N(C(=O)/C(C#N)=C/C(C)(C)C)CC3)C2(CC2)C1] | C88 |
| 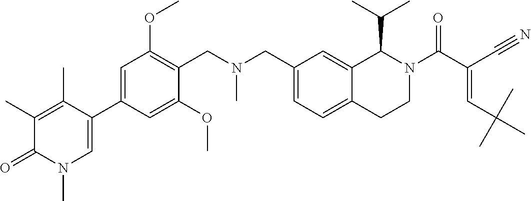 [SMILES: COc1cc(-c2cn(C)c(=O)c(C)c2C)cc(OC)c1CN(C)Cc1ccc2c(c1)[C@@H](C(C)C)N(C(=O)/C(C#N)=C/C(C)(C)C)CC2] | C89 |
| 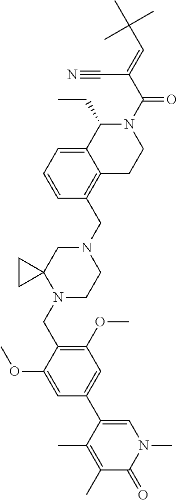 | C90 |
| 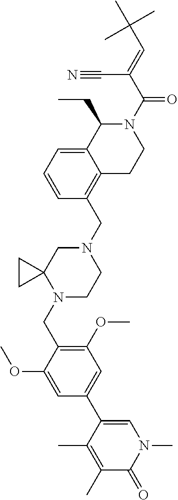 | C91 |
| 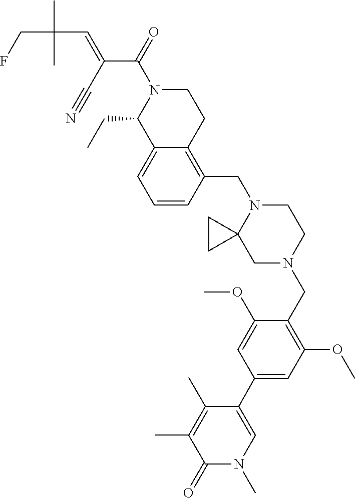 [SMILES: CC[C@H]1c2cccc(CN3CCN(Cc4c(OC)cc(-c5cn(C)c(=O)c(C)c5C)cc4OC)CC34CC4)c2CCN1C(=O)/C(C#N)=C/C(C)(C)CF] | C92 |
| 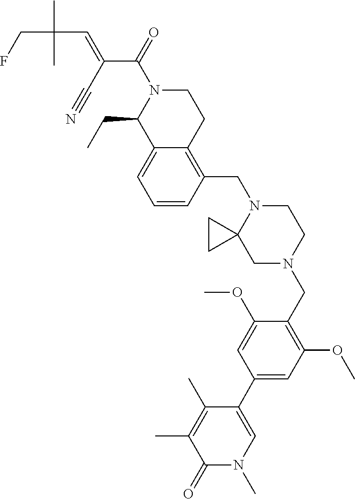 [SMILES: CC[C@@H]1c2cccc(CN3CCN(Cc4c(OC)cc(-c5cn(C)c(=O)c(C)c5C)cc4OC)CC34CC4)c2CCN1C(=O)/C(C#N)=C/C(C)(C)CF] | C93 |
| 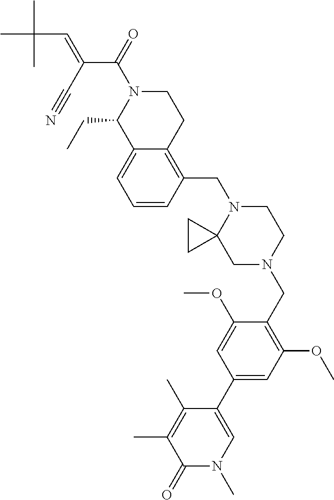 [SMILES: CC[C@H]1c2cccc(CN3CCN(Cc4c(OC)cc(-c5cn(C)c(=O)c(C)c5C)cc4OC)CC34CC4)c2CCN1C(=O)/C(C#N)=C/C(C)(C)C] | C94 |
| 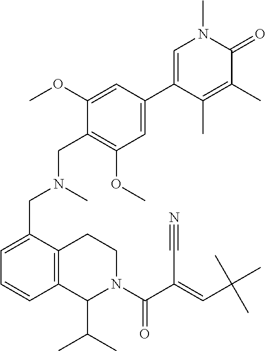 [SMILES: COc1cc(-c2cn(C)c(=O)c(C)c2C)cc(OC)c1CN(C)Cc1cccc2c1CCN(C(=O)/C(C#N)=C/C(C)(C)C)C2C(C)C] | C95 |
| 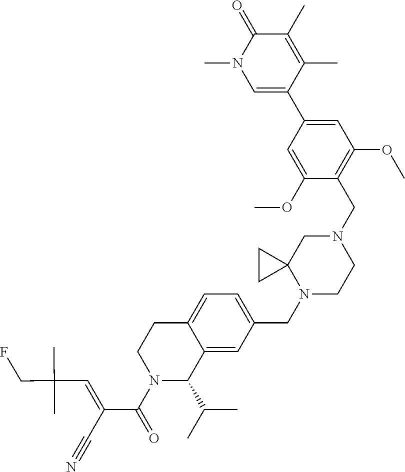 [SMILES: COc1cc(-c2cn(C)c(=O)c(C)c2C)cc(OC)c1CN1CCN(Cc2ccc3c(c2)[C@H](C(C)C)N(C(=O)/C(C#N)=C/C(C)(C)CF)CC3)C2(CC2)C1] | C96 |
| 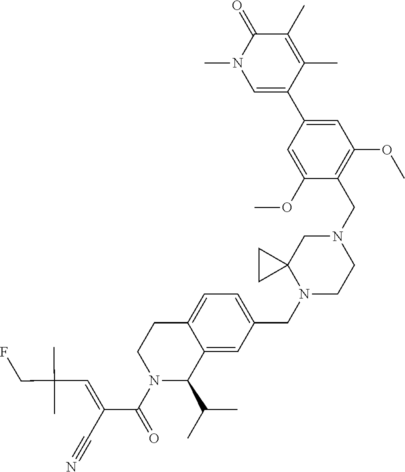 [SMILES: COc1cc(-c2cn(C)c(=O)c(C)c2C)cc(OC)c1CN1CCN(Cc2ccc3c(c2)[C@@H](C(C)C)N(C(=O)/C(C#N)=C/C(C)(C)CF)CC3)C2(CC2)C1] | C97 |
| 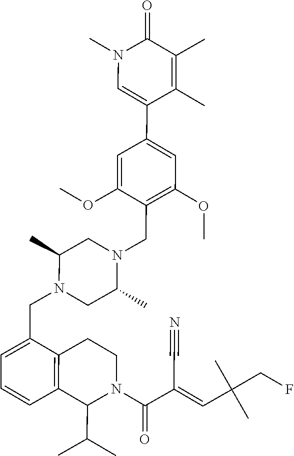 [SMILES: COc1cc(-c2cn(C)c(=O)c(C)c2C)cc(OC)c1CN1C[C@H](C)N(Cc2cccc3c2CCN(C(=O)/C(C#N)=C/C(C)(C)CF)C3C(C)C)C[C@H]1C] | C98 |
| 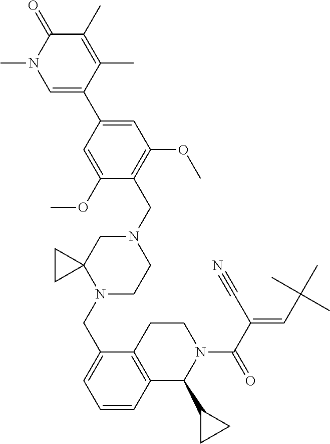 [SMILES: COc1cc(-c2cn(C)c(=O)c(C)c2C)cc(OC)c1CN1CCN(Cc2cccc3c2CCN(C(=O)/C(C#N)=C/C(C)(C)C)[C@H]3C2CC2)C2(CC2)C1] | C99 |
| 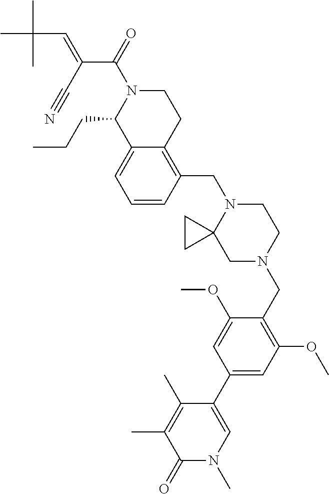 [SMILES: CCC[C@H]1c2cccc(CN3CCN(Cc4c(OC)cc(-c5cn(C)c(=O)c(C)c5C)cc4OC)CC34CC4)c2CCN1C(=O)/C(C#N)=C/C(C)(C)C] | C100 |
| 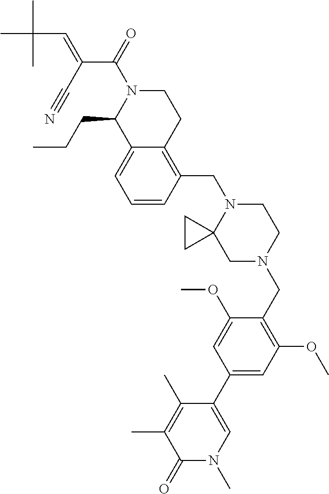 [SMILES: CCC[C@@H]1c2cccc(CN3CCN(Cc4c(OC)cc(-c5cn(C)c(=O)c(C)c5C)cc4OC)CC34CC4)c2CCN1C(=O)/C(C#N)=C/C(C)(C)C] | C101 |
| 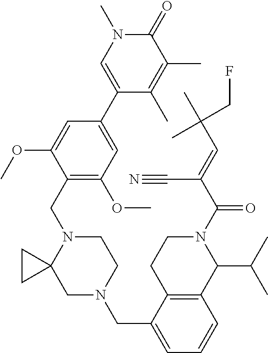 [SMILES: COc1cc(-c2cn(C)c(=O)c(C)c2C)cc(OC)c1CN1CCN(Cc2cccc3c2CCN(C(=O)/C(C#N)=C/C(C)(C)CF)C3C(C)C)CC12CC2] | C102 |
| 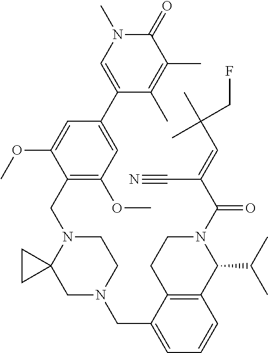 [SMILES: COc1cc(-c2cn(C)c(=O)c(C)c2C)cc(OC)c1CN1CCN(Cc2cccc3c2CCN(C(=O)/C(C#N)=C/C(C)(C)CF)[C@@H]3C(C)C)CC12CC2] | C103 |
| 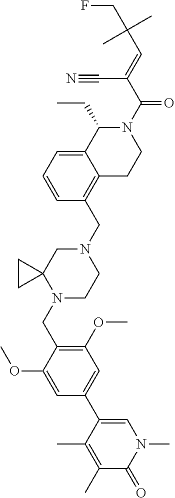 | C104 |
| 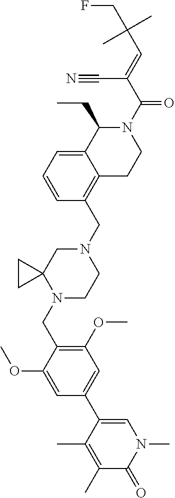 | C105 |
| 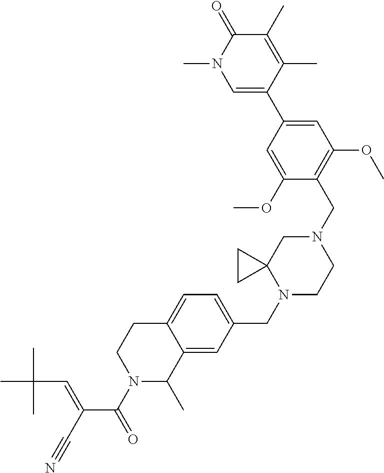 [SMILES: COc1cc(-c2cn(C)c(=O)c(C)c2C)cc(OC)c1CN1CCN(Cc2ccc3c(c2)C(C)N(C(=O)/C(C#N)=C/C(C)(C)C)CC3)C2(CC2)C1] | C106 |
| 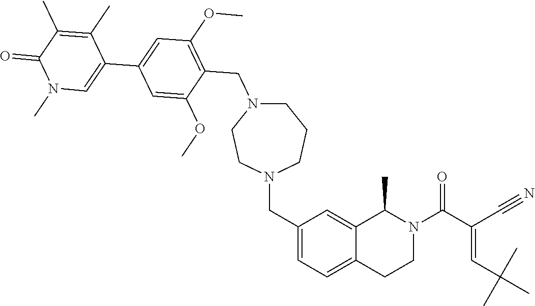 [SMILES: COc1cc(-c2cn(C)c(=O)c(C)c2C)cc(OC)c1CN1CCCN(Cc2ccc3c(c2)[C@@H](C)N(C(=O)/C(C#N)=C/C(C)(C)C)CC3)CC1] | C107 |

Table 1 showing structures of exemplary bifunctional molecules A2 to A76, BRD9a to BRD9ac, B1 to B84, B86, B88 to B96, B98 to B104, B106 to B127, B130 to B149, B152 to B156, B158 to B162, B164, B165, B169, B173 to B175, B180 to B215, and C1 to C107.

Table 1 shows indicative structures of the exemplified examples. Absolute stereochemistry and double bond geometry, as appropriate, is arbitrarily assigned unless otherwise indicated herein, for example, in the detailed experimental section.

In some more specific examples, the bifunctional molecule is any one of formulae B202, C6, C35, and C77.

### Isotopically-Labelled Compounds

The disclosure also encompasses various deuterated forms of the compounds of any of the formulae disclosed herein, including formulae (I), (II), (III), (WZI) to (WZV), (WI), (WII), (WIII), (WIV), (WV), (A), (A1) to (A3), 1T, 2T, 3T, 4T, 5T, 6T, 7T, 8T, 9T, 11T, 12T, 13T, 14T (including corresponding subgeneric formulae defined herein) or a pharmaceutically acceptable salt and/or a corresponding tautomer form thereof (including subgeneric formulae, as defined above) of the present disclosure. Each available hydrogen atom attached to a carbon atom may be independently replaced with a deuterium atom. A person of ordinary skill in the art will know how to synthesize deuterated forms of the compounds of any of the formulae disclosed herein, including those referred to above. For example, deuterated materials, such as alkyl groups may be prepared by conventional techniques (see for example: methyl-d₃-amine available from Aldrich Chemical Co., Milwaukee, WI, Cat. No. 489,689-2).

The disclosure also includes isotopically-labelled compounds which are identical to those recited in any of the formulae disclosed herein, including formulae (I), (II), (III), (WZI) to (WZV), (WI), (WII), (WIII), (WIV), (WV), (A), (A1) to (A3), 1a, 1a′, 1b, 1c, 1b′, 1a¹, 1a², 1a³, 1e, 1f, 1g, 1f′, 1g′, 1ea to 1eh, 1fa to 1fh, 1ga, 1ea′, 1h to 1z or 2a to 2g (including corresponding subgeneric formulae defined herein) or a pharmaceutically acceptable salt and/or a corresponding tautomer form thereof (including subgeneric formulae, as defined above) of the present disclosure, but for the fact that one or more atoms are replaced by an atom having an atomic mass or mass number different from the atomic mass or mass number most commonly found in nature. Examples of isotopes that can be incorporated into compounds of the disclosure include isotopes of hydrogen, carbon, nitrogen, oxygen, fluorine, iodine and chlorine such as ²H, ³H, ¹¹C, ¹³C, ¹⁴C, ¹⁸F, ¹²³I or ¹²⁵I. Compounds of the present disclosure and pharmaceutically acceptable salts of said compounds that contain the aforementioned isotopes and/or other isotopes of other atoms are within the scope of the present disclosure. Isotopically labelled compounds of the present disclosure, for example those into which radioactive isotopes such as ³H or ¹⁴C have been incorporated, are useful in drug and/or substrate tissue distribution assays. Tritiated, i.e. ³H, and carbon-14, i.e. ¹⁴C, isotopes are particularly preferred for their ease of preparation and detectability. ¹¹C and ¹⁸F isotopes are particularly useful in PET (positron emission tomography).

### Degradation Activity

Degradation may be determined by measuring the amount of a BRD9 target protein in the presence of a bifunctional molecule as described herein and/or comparing this to the amount of the BRD9 target protein observed in the absence of the bifunctional molecule. For example, the amount of BRD9 target protein in a cell that has been contacted and/or treated with a bifunctional molecule as described herein may be determined. This amount may be compared to the amount of BRD9 target protein in a cell that has not been contacted and/or treated with the bifunctional molecule. If the amount of BRD9 target protein is decreased in the cell contacted and/or treated with the bifunctional molecule, the bifunctional molecule may be considered as facilitating and/or promoting the degradation and/or proteolysis of the BRD9 target protein.

The amount of the BRD9 target protein can be determined using methods known in the art, for example, by performing immunoblotting assays, Western blot analysis and/or ELISA with cells that have been contacted and/or treated with a bifunctional molecule.

Selective degradation and/or increased proteolysis may be considered to have occurred if at least a 10% decrease in the amount of a BRD9 target protein is observed, for example, 20%, 30%, 40%, 50%, 60%, 70%, 80%, 90% or 100% following administration of the bifunctional molecule to the cell.

For example, selective degradation and/or increased proteolysis may be considered to have occurred if at least a 10% decrease in the amount of a BRD9 target protein is observed, (e.g. at least 20%, 30%, 40%, 50%, 60%, 70%, 80%, 90% or 100% decrease) within 4 hours or more (e.g. 4 hours, 8 hours, 12 hours, 24 hours, 30 hours, 36 hours, 42 hours, 48 hours, 54 hours, 60 hours, 66 hours and 72 hours) following administration of the bifunctional molecule to the cell. In particular examples, selective degradation and/or increased proteolysis is considered to have occurred if at least a 40% decrease in the amount of a BRD9 target protein is observed. The bifunctional molecule may be administered at any concentration, e.g. a concentration between 0.01 nM to 10 μM, such as 0.01 nM, 0.1 nM, 1 nM, 10 nM, 100 nM, 1 μM, and 10 μM. In some instances, an increase of at least 30%, 40%, 50%, 60%, 70%, 80%, 90%, or approximately 100% in the degradation of the BRD9 target protein is observed following administration of the bifunctional molecule at a concentration of approximately 100 nM (e.g. following an incubation period of approximately 8 hours).

One measure of degrader activity of the bifunctional molecules is the DC₅₀ value. As used herein, DC₅₀ is the concentration required to reach 50% of the maximal degradation of the BRD9 target protein. The bifunctional molecules described herein may comprise a DC₅₀ of less than or equal to 10000 nM, less than or equal to 1000 nM, less than or equal to 500 nM, less than or equal to 100 nM or less than or equal to 75 nM. In some cases, the bifunctional molecules comprise a DC₅₀ less than or equal to 50 nM, less than or equal to 25 nM, less than or equal to 10 nM, less than or equal to 5 nM, less than or equal to 1.5 nM, less than or equal to 1 nM, or less than or equal to 0.5 nM. In some cases, the bifunctional molecules of the invention comprise a DC₅₀ of less than 1.25 nM in either of the BRD9 degradation assays described below.

Another measure of the degrader activity of the bifunctional molecules is the Dₘₐₓ value. As used herein, Dₘₐₓ represents the maximal percentage of BRD9 target protein degradation. The bifunctional molecules described herein may comprise a Dₘₐₓ of at least 10%, at least 20%, at least 25%, at least 30%, at least 40%, at least 50%, at least 60%, at least 70%, at least 80%, at least 90%, at least 95% or about 100%. In particular examples, the bifunctional molecules comprise a Dₘₐₓ of at least 40%. In some cases, the bifunctional molecules of the invention comprise a DC₅₀ of less than 1.25 nM and a Dₘₐₓ of 75% or more in either of the BRD9 degradation assays described below.

In some cases, the bifunctional molecules of the invention comprise a Dₘₐₓ of 75% or more in either of the BRD9 degradation assays described below.

Yet another measure of the efficacy of the described bifunctional molecules may be their effect on cell viability and/or their IC₅₀ value. For example, an anti-proliferative effect of a bifunctional molecule as described herein may be assessed in a cell viability assay to provide an IC₅₀ value. As used herein, the IC₅₀ value represents the concentration at which 50% cell viability was observed in the cell viability assay (following administration of a bifunctional molecule as described herein). In terms of cell viability, the bifunctional molecules described herein may comprise an IC₅₀ of less than 1000 nM, less than 500 nM, less than 100 nM, less than 50 nM, less than 25 nM, less than 20 nM, or less than 10 nM. In some cases, the bifunctional molecules described herein may comprise an IC₅₀ value of less than 5 nM.

### Bioavailability

The bifunctional molecules described herein may provide degraders with improved levels of bioavailability, such as improved levels of oral bioavailability.

As used herein, bioavailability is a fraction or proportion of an administered active agent (e.g. a bifunctional molecule as described herein) that reaches the systemic circulation in a subject. As used herein, oral bioavailability is a fraction or proportion of an orally administered active agent that reaches the systemic circulation in a subject.

Oral bioavailability is calculated by comparing the area under the curve (AUC) for an intravenous administration of a particular active agent to the AUC for an oral administration of that active agent. The AUC value is the definite integral of a curve that shows the variation of active agent concentration in the blood plasma as a function of time. As used herein, AUC_{0-INF} is the area under the curve from time zero which has been extrapolated to infinity and represents the total active agent exposure over time

Oral bioavailability (F) may be calculated using the following formula:

$$F = 100 \cdot \frac{{AUC}_{po} \cdot {D}_{iv}}{{AUC}_{iv} \cdot {D}_{po}}$$

Wherein:
Dᵢᵥ=dose administered intravenously;
Dₚₒ=dose administered orally;
AUCᵢᵥ=Area under the curve from time zero to infinity following intravenous administration; and
AUCₚₒ=Area under the curve from time zero to infinity following oral administration.

The bifunctional molecules described herein may have an oral bioavailability of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%. In some cases, the oral bioavailability of a bifunctional molecule as described herein may be approximately 28%.

### Pharmaceutical Compositions

The present disclosure provides a pharmaceutical composition comprising the bifunctional molecules described herein. In such compositions, the bifunctional molecule may be suitably formulated such that it can be introduced into the environment of the cell by a means that allows for a sufficient portion of the molecule to enter the cell to induce degradation of the BRD9 target protein.

Accordingly, there is provided a pharmaceutical composition comprising a bifunctional molecule as described herein together with a pharmaceutically acceptable carrier.

Pharmaceutically acceptable carriers are well known to those skilled in the art and include, but are not limited to, phosphate buffer solutions and/or saline. Pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

In addition to the aforementioned carrier ingredients the pharmaceutical compositions described above may alternatively or additionally include, an appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Pharmaceutical compositions may be present in any formulation typical for the administration of a pharmaceutical compound to a subject. Representative examples of typical formulations include, but are not limited to, capsules, granules, tablets, powders, lozenges, suppositories, pessaries, nasal sprays, gels, creams, ointments, sterile aqueous preparations, sterile solutions, aerosols, implants etc.

A pharmaceutical composition is formulated to be compatible with its intended route of administration. Examples of routes of administration include parenteral, e.g., intravenous, intradermal, subcutaneous, oral, transdermal, topical, transmucosal, vaginal and rectal administration.

The pharmaceutical compositions may include those suitable for oral, parenteral (including subcutaneous, intradermal, intramuscular and intravenous), topical (including dermal, buccal and sublingual), rectal, nasal and pulmonary administration e.g., by inhalation. The composition may, where appropriate, be conveniently presented in discrete dosage units and may be prepared by any of the methods well known in the art of pharmacy. Methods typically include the step of bringing into association an active compound with liquid carriers or finely divided solid carriers or both and then, if necessary, shaping the product into the desired formulation.

Pharmaceutical compositions suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. The bifunctional molecules may also be formulated as dispersible granules, which may for example be suspended in water before administration, or sprinkled on food. The granules may be packaged, e.g., in a sachet. Compositions suitable for oral administration wherein the carrier is a liquid may be presented as a solution or a suspension in an aqueous or non-aqueous liquid, or as an oil-in-water liquid emulsion. Compositions for oral administration include controlled release dosage forms, e.g., tablets wherein an active compound is formulated in an appropriate release-controlling matrix, or is coated with a suitable release-controlling film.

Pharmaceutical compositions suitable for parenteral administration include sterile solutions or suspensions of an active compound in aqueous or oleaginous vehicles. Injectable preparations may be adapted for bolus injection or continuous infusion. Such preparations are conveniently presented in unit dose or multi-dose containers, which are sealed after introduction of the formulation until required for use. Alternatively, the bifunctional molecule may be in powder form, which is constituted with a suitable vehicle, such as sterile, pyrogen-free water, before use.

The pharmaceutical composition may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins.

Pharmaceutical compositions suitable for topical formulation may be provided for example as gels, creams or ointments.

The bifunctional molecules described herein may be present in the pharmaceutical compositions as a pharmaceutically and/or physiologically acceptable salt, solvate or derivative.

Representative examples of pharmaceutically and/or physiologically acceptable salts of the bifunctional molecules of the disclosure may include, but are not limited to, acid addition salts formed with organic carboxylic acids such as acetic, lactic, tartaric, maleic, citric, pyruvic, oxalic, fumaric, oxaloacetic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids.

Pharmaceutically and/or physiologically functional derivatives of compounds of the present invention are derivatives, which may be converted in the body into the parent compound. Such pharmaceutically and/or physiologically functional derivatives may also be referred to as “pro-drugs” or “bioprecursors”. Pharmaceutically and/or physiologically functional derivatives of compounds of the present disclosure may include hydrolysable esters or amides, particularly esters, in vivo.

It may be convenient or desirable to prepare, purify, and/or handle a corresponding pharmaceutically and/or physiologically acceptable solvate of the bifunctional molecules described herein, which may be used in the any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

### Uses of Moiety Z

As described herein, the moiety Z may form part of a bifunctional molecule intended for use in a method of targeted protein degradation, wherein the moiety Z acts to modulate, facilitate and/or promote proteasomal degradation of the BRD9 target protein.

As such, according to a further aspect of the disclosure, there is provided a use of the moiety Z or a compound comprising moiety Z (e.g. as defined in any one of formula (I) to (III)) in a method of BRD9 degradation (e.g. an in vitro or in vivo method of targeted protein degradation). For example, moiety Z may find particular application as a promoter or facilitator of BRD9 degradation. There is also provided a use of moiety Z or a compound comprising moiety Z (e.g. as defined in any one of formula (I) to (III)) in the manufacture of a bifunctional molecule suitable for BRD9 degradation.

### Therapeutic Methods and Uses

The bifunctional molecules of the present disclosure may modulate, facilitate and/or promote proteasomal degradation of a BRD9 target protein. As such, there is provided a method of selectively degrading and/or increasing proteolysis of a BRD9 target protein in a cell, the method comprising contacting and/or treating the cell with a bifunctional molecule as described herein. The method may be carried out in vivo or in vitro.

In particular, there is provided a method of selectively degrading and/or increasing proteolysis of a BRD9 target protein in a subject in need thereof, the method comprising administering to the subject a therapeutically effective amount of a bifunctional molecule of the present disclosure. As such, the bifunctional molecules of the present disclosure may find application in medicine and/or therapy. Specifically, the bifunctional molecules of the present disclosure may find use in the treatment and/or prevention of any disease or condition, which is modulated through the BRD9 target protein. For example, the bifunctional molecules of the present disclosure may be useful in the treatment of any disease, which is modulated through the BRD9 target protein by lowering the level of that protein in the cell, e.g. cell of a subject.

There is further provided the use of the bifunctional molecules as described herein in the manufacture of a medicament for the treatment and/or prevention of any disease or condition, which is modulated through the BRD9 target protein. Additionally, there is provided the use of a moiety Z (e.g as defined in any one of formulae (I) to (III) in the manufacture of a medicament for the treatment and/or prevention of any disease or condition, which is modulated through the BRD9 target protein.

Diseases and/or conditions that may be treated and/or prevented by the molecules of the disclosure include any disease, which is associated with and/or is caused by an abnormal level of BRD9 protein activity.

Such diseases and conditions include those whose pathology is related at least in part to an abnormal (e.g. elevated) level of a BRD9 protein and/or the overexpression of an BRD9 protein. For example, the bifunctional molecules may find use in the treatment and/or prevention of diseases where an elevated level of a BRD9 protein is observed in a subject suffering from the disease. In other examples, the diseases and/or conditions may be those whose pathology is related at least in part to inappropriate BRD9 protein expression (e.g., expression at the wrong time and/or in the wrong cell), or excessive BRD9 protein expression.

Accordingly, there is provided a method of treating and/or preventing a disease or condition, which is associated with and/or is caused by an abnormal level of BRD9 protein activity, which comprises administering a therapeutically effective amount of a bifunctional compound as described herein. Representative examples of the diseases and/or conditions that may be treated and/or prevented by the use of the described bifunctional compounds include (but are not limited to) cancer.

A recent review article summarises the potential mechanisms of action of BRD9 in carcinogenesis and also describes various strategies for targeting BRD9 for use as cancer treatments (Zhu et al, OncoTargets and Therapy, 2020, Vol. 13, pages 13191-13200). Previous studies have shown that BRD9 is essential for the proliferation of SMARCB1-deficient cancer cell lines, suggesting it is a therapeutic target for these cancers. (Xiaofeng Wang et. al., Nature Communications, 2019, 10 (1881)). Recent studies also highlight a role of BRD9 in leukemia growth: BRD9 was shown to be required for the proliferation of acute myeloid leukemia (AML) cells (Nature Chemical Biology, 2016, 101038/nchembio.2115). In addition to the role of BRD9 as a functional dependency in certain cancers, BRD9 also plays a pivotal role in immune cells as a regulator of regulatory T cells (Tregs) via transcriptional control of Foxp3 target genes, “BioRxiv, 10.1101/2020.02.26.964981.

Representative examples of cancers that may be treated and/or prevented using the described bifunctional molecules include, but are not limited to:
(i) brain tumours such as for example acoustic neurinoma, astrocytomas such as pilocytic astrocytomas, fibrillary astrocytoma, protoplasmic astrocytoma, gemistocytary astrocytoma, anaplastic astrocytoma and glioblastoma, brain lymphomas, brain metastases, hypophyseal tumour such as prolactinoma, HGH (human growth hormone) producing tumour and ACTH producing tumour (adrenocorticotropic hormone), craniopharyngiomas, medulloblastomas, meningeomas and oligodendrogliomas;
(ii) nerve tumours (neoplasms) such as for example tumours of the vegetative nervous system such as neuroblastoma sympathicum, ganglioneuroma, paraganglioma (pheochromocytoma, chromaffinoma) and glomus-caroticum tumour, tumours on the peripheral nervous system such as amputation neuroma, neurofibroma, neurinoma (neurilemmoma, Schwannoma) and malignant Schwannoma, as well as tumours of the central nervous system such as brain and bone marrow tumours;
(iii) intestinal cancer such as for example carcinoma of the rectum, colon carcinoma, colorectal carcinoma, anal carcinoma, carcinoma of the large bowel, tumours of the small intestine and duodenum;
(iv) eyelid tumours such as basalioma or basal cell carcinoma;
(v) pancreatic cancer or carcinoma of the pancreas;
(vi) bladder cancer or carcinoma of the bladder;
(vii) lung cancer (bronchial carcinoma) such as for example small-cell bronchial carcinomas (oat cell carcinomas) and non-small cell bronchial carcinomas (NSCLC) such as plate epithelial carcinomas, adenocarcinomas and large-cell bronchial carcinomas;
(viii) breast cancer such as for example mammary carcinoma such as infiltrating ductal carcinoma, colloid carcinoma, lobular invasive carcinoma, tubular carcinoma, adenocystic carcinoma and papillary carcinoma;
(ix) non-Hodgkin's lymphomas (NHL) such as for example Burkitt's lymphoma, low-malignancy non-Hodgkin's lymphomas (NHL) and mucosis fungoides;
(x) uterine cancer or endometrial carcinoma or corpus carcinoma;
(xi) CUP syndrome (Cancer of Unknown Primary);
(xii) ovarian cancer or ovarian carcinoma such as mucinous, endometrial or serous cancer;
(xiii) gall bladder cancer;
(xiv) bile duct cancer such as for example Klatskin tumour;
(xv) testicular cancer such as for example seminomas and non-seminomas;
(xvi) lymphoma (lymphosarcoma) such as for example malignant lymphoma, Hodgkin's disease, non-Hodgkin's lymphomas (NHL) such as chronic lymphatic leukaemia, leukaemic reticuloendotheliosis, immunocytoma, plasmocytoma (multiple myeloma (MM)), immunoblastoma, Burkitt's lymphoma, T-zone mycosis fungoides, large-cell anaplastic lymphoblastoma and lymphoblastoma;
(xvii) laryngeal cancer such as for example tumours of the vocal cords, supraglottal, glottal and subglottal laryngeal tumours;
(xviii) bone cancer such as for example osteochondroma, chondroma, chondroblastoma, chondromyxoid fibroma, osteoma, osteoid osteoma, osteoblastoma, eosinophilic granuloma, giant cell tumour, chondrosarcoma, osteosarcoma, Ewing's sarcoma, reticulo-sarcoma, plasmocytoma, fibrous dysplasia, juvenile bone cysts and aneurysmatic bone cysts;
(xix) head and neck tumours such as for example tumours of the lips, tongue, floor of the mouth, oral cavity, gums, palate, salivary glands, throat, nasal cavity, paranasal sinuses, larynx and middle ear;
(xx) liver cancer such as for example liver cell carcinoma or hepatocellular carcinoma (HCC);
(xxi) leukaemias, such as for example acute leukaemias such as acute lymphatic/lymphoblastic leukaemia (ALL), acute myeloid leukaemia (AML); chronic leukaemias such as chronic lymphatic leukaemia (CLL), chronic myeloid leukaemia (CML);
(xxii) stomach cancer or gastric carcinoma such as for example papillary, tubular and mucinous adenocarcinoma, signet ring cell carcinoma, adenosquamous carcinoma, small-cell carcinoma and undifferentiated carcinoma;
(xxiii) melanomas such as for example superficially spreading, nodular, lentigo-maligna and acral-lentiginous melanoma;
(xxiv) renal cancer such as for example kidney cell carcinoma or hypemephroma or Grawitz's tumour;
(xxv) oesophageal cancer or carcinoma of the oesophagus;
(xxvi) penile cancer;
(xxvii) prostate cancer;
(xxviii) throat cancer or carcinomas of the pharynx such as for example nasopharynx carcinomas, oropharynx carcinomas and hypopharynx carcinomas;
(xxix) retinoblastoma such as for example vaginal cancer or vaginal carcinoma;
(xxx) plate epithelial carcinomas, adenocarcinomas, in situ carcinomas, malignant melanomas and sarcomas;
(xxxi) thyroid carcinomas such as for example papillary, follicular and medullary thyroid carcinoma, as well as anaplastic carcinomas;
(xxxii) spinalioma, epidermoid carcinoma and plate epithelial carcinoma of the skin;
(xxxiii) thymomas, cancer of the urethra and cancer of the vulva.

In specific examples, the cancer is any one selected from the group consisting of hematopoietic malignancies (including but not limited to AML, MM) and solid tumors including but not limited to lung, liver, colon, brain, thyroid, pancreas, breast, ovary and prostate cancer.

Other particular examples of cancers that may be treated by a targeted protein degradation of BRD9 may include cancers that harbour SMARCB1 abnormalities, for example SMARCB1-deficient cancers, such as malignant rhabdoid tumors and several specific types of sarcoma, as well as leukemia such as acute myeloid leukemia (AML).

As used herein, the term “patient” or “subject” is used to describe an animal, such as a mammal (e.g. a human or a domesticated animal), to whom treatment, including prophylactic treatment, with the compositions according to the present disclosure is provided. For treatment of those infections, conditions or disease states which are specific to a specific animal such as a human patient, the term patient refers to that specific animal, including a domesticated animal such as a dog or cat or a farm animal such as a horse, cow, sheep, etc. In general, in the present invention, the term patient refers to a human patient unless otherwise stated or implied from the context of the use of the term.

### Assays

The disclosure also encompasses a method of screening bifunctional molecules to identify suitable BRD9 binding ligands and linkers for use in the bifunctional molecules described herein, e.g. a bifunctional molecule that is able to effectively modulate, facilitate and/or promote proteolysis of a BRD9 target protein. This method may assist in identifying suitable linkers for a particular BRD9 binding partner such that the level of degradation is further optimised.

The method may comprise:
a. providing a bifunctional molecule comprising:
   (i) a first ligand comprising a structure according to Z (e.g. as defined in any one of the formulae defined herein, including (I), (II), (III), (WI), (WII), (WIII), (WIV), (WV) and any sub-generic formulae);
   (ii) a second ligand that binds to a BRD9 target protein (e.g. a BRD9 binding ligand as defined in any one of the formulae defined herein, including any one of formulae 1a, 1a′, 1b, 1c, 1b′, 1a¹, 1a², 1a³, 1e, 1f, 1g, 1f′, 1g′, 1ea to 1eh, 1fa to 1fh, 1ga, 1ea′, 1h to 1z, 2a to 2g and any sub-generic formulae); and
   (iii) a linker that covalently attaches the first and second ligands;
b. contacting a cell with the bifunctional molecule; and
c. detecting degradation of the BRD9 target protein in the cell.

This method may further comprise the steps of
d. detecting degradation of the BRD9 target protein in the cell in the absence of the bifunctional molecule; and
e. comparing the level of degradation of the BRD9 target protein in the cell contacted with the bifunctional molecule to the level of degradation of the BRD9 target protein in the absence of the bifunctional molecule;
   wherein an increased level of degradation of the BRD9 target protein in the cell contacted with the bifunctional molecule indicates that the bifunctional molecule has facilitated and/or promoted the degradation of the BRD9 target protein.

In such methods, a step of detecting degradation of the BRD9 target protein may comprise detecting changes in levels of BRD9 protein in a cell. For example, a reduction in the level of the BRD9 protein indicates degradation of the BRD9 protein. An increased reduction in the level of the BRD9 protein in the cell contacted with the bifunctional molecule (compared to any reduction in the levels of BRD9 protein observed in the cell in the absence of the bifunctional molecule) indicates that the bifunctional molecule has facilitated and/or promoted the degradation of the BRD9 target protein.

The method may further comprise providing a plurality of linkers, each one being used to covalently attach the first and second ligands together to form a plurality of bifunctional molecules.

The level of degradation provided by each one of the plurality of bifunctional molecules may be detected and compared. Those bifunctional molecules showing higher levels of BRD9 protein degradation indicate preferred and/or optimal linkers for use with the selected BRD9 protein binding partner.

The method may be carried out in vivo or in vitro.

### Compound Library

The disclosure also provides a library of bifunctional molecules, the library comprising a plurality of bifunctional molecules, the plurality of bifunctional molecules comprising a plurality of Z moieties covalently linked to a selected BRD9 protein binding partner.

As such, the BRD9 binding partner may be pre-selected and the Z moiety may not be determined in advance. The library may be used to determine the activity of a candidate Z moiety of a bifunctional molecule in modulating, promoting and/or facilitating selective protein degradation of a BRD9 protein.

The disclosure also includes a library of bifunctional molecules, the library comprising a plurality of bifunctional molecules, the plurality of bifunctional molecules comprising a plurality of BRD9 protein binding ligands and a selected Z moiety. As such, the Z moiety of the bifunctional molecule may be pre-selected and the BRD9 target protein may not be determined in advance. The library may be used to determine the activity of a putative BRD9 protein binding ligand and its value as a binder of a BRD9 protein to facilitate BRD9 degradation.

### Methods of Manufacture

According to a further aspect of the disclosure, there is provided a method of making a bifunctional molecule as described herein.

The method of making the bifunctional molecule may comprise the steps of:
(a) providing a first ligand or moiety comprising a structure according to Z (e.g. as defined in any one of the formulae defined herein, including (I), (II), (III), (WI), (WII), (WIII), (WIV), (WV) and any sub-generic formulae);
(b) providing a second ligand or moiety that binds to a BRD9 protein (e.g. a BRD9 binding ligand as defined in any one of the formulae defined herein, including any one of formulae 1a, 1a′, 1b, 1c, 1b′, 1a¹, 1a², 1a³, 1e, if, 1g, 1f′, 1g′, 1ea to 1eh, 1fa to 1fh, 1ga, 1ea′, 1h to 1z, 2a to 2g and any sub-generic formulae); and
(c) linking (e.g. covalently linking) the first and second ligands or moieties using a linker as defined herein.

In other examples, the method of making the bifunctional molecule may comprise the steps of:
(a) providing a BRD9 protein binding ligand (e.g. a BRD9 binding ligand as defined in any one of the formulae defined herein, including any one of formulae 1a, 1a′, 1b, 1c, 1b′, 1a¹, 1a², 1a³, 1e, 1f, 1g, 1f′, 1g′, 1ea to 1eh, 1fa to 1fh, 1ga, 1ea′, 1h to 1z, 2a to 2g and any sub-generic formulae);
(b) linking (e.g. covalently linking) a linker (as defined herein) to the BRD9 protein binding ligand to provide a BRD9 protein binding ligand-linker conjugate (TBL-L);
(c) further reacting the linker moiety of the conjugate to add and/or form a structure according to Z (e.g. as defined in any one of the formulae defined herein, including (I), (II), (III), (W), (WII), (WIII), (WIV), (WV) and any sub-generic formulae) thereon to provide the bifunctional molecule having the general formula TBL-L-Z.

### Kit of Parts

According to a further aspect of the disclosure, there is provided a kit of separate parts from which the bifunctional molecules defined herein may be prepared, for example according to the methods of manufacture defined above.

The kit of parts may comprise:
(i) a first ligand comprising a structure according to Z as defined above (e.g. as defined in any one of the formulae defined herein, including (I), (II), (III), (WI), (WII), (WIII), (WIV), (WV) and any sub-generic formulae);
(ii) a second ligand that binds to BRD9 as defined above (e.g. a BRD9 binding ligand as defined in any one of the formulae defined herein, including any one of formulae 1a, 1a′, 1b, 1c, 1b′, 1a¹, 1a², 1a³, 1e, 1f, 1g, 1f′, 1g′, 1ea to 1eh, 1fa to 1fh, 1ga, 1ea′, 1h to 1z, 2a to 2g and any sub-generic formulae); and
(iii) a linker that covalently attaches the first and second ligands as defined above.

In some cases, each of the first ligand, second ligand and linker are separate from one another.

### CLAUSES

The present disclosure may also be defined with reference to the following set of clauses:

1. A bifunctional molecule comprising the general formula:

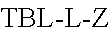

wherein TBL is a target protein binding ligand that binds BRD9;
L is a linker; and
Z comprises a structure according to formula (I):

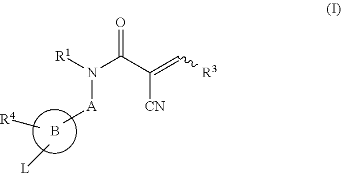

wherein
R¹ is selected from C₁ to C₆ alkyl, benzyl, substituted benzyl, carbocyclyl, substituted carbocyclyl, heterocyclyl and substituted heterocyclyl, optionally wherein the C₁ to C₆ alkyl is substituted with one or more heteroatoms selected from halo, N, O and S and/or is substituted with a carbocyclic or heterocyclic group;
A is absent or is CR²R^{2′};
B is selected from aryl, heteroaryl, substituted aryl and substituted heteroaryl;
R² and R^{2′} are each independently selected from H and C₁ to C₆ alkyl, optionally wherein the C₁ to C₆ alkyl is substituted with one or more heteroatoms selected from N, O or S, or wherein R² and R^{2′} together form a 3-, 4-, 5- or 6-membered carbocyclic or heterocyclic ring;
R³ is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S;
R⁴ is H, C₁ to C₆ alkyl, optionally wherein the C₁ to C₆ alkyl is substituted with one or more heteroatoms selected from N, O or S;
or wherein R¹ and R⁴ together form a 5-, 6-, or 7-membered heterocyclic ring;
or wherein when A is CR²R^{2′}:
R¹ and R² together form a 5-, 6-, or 7-membered heterocyclic ring; or
R² and R⁴ together form a 5-, 6-, or 7-membered heterocyclic or carbocyclic ring;
wherein L shows the point of attachment of the linker;
or
Z comprises a structure according to formula (WZI):

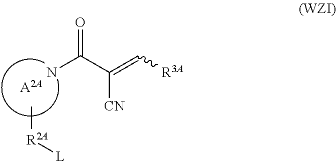

wherein:
ring A^{2A} is an optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from N, O and S;
R^{2A} is absent or is selected from aryl, substituted aryl, heteroaryl, substituted heteroaryl, heterocycloalkyl, substituted heterocycloalkyl, NR^{y}, —CH(aryl)-, —CH(substituted aryl)-, —CH(heteroaryl)- and —CH(substituted heteroaryl)-;
wherein R^{y} is optionally substituted C₁₋₆alkyl or H;
R^{3A} is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S; and
L shows the point of attachment of the linker;
or
Z comprises a structure according to formula (WI):

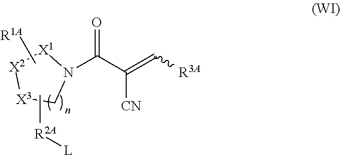

wherein R^{1A} is absent or is selected from aryl, substituted aryl, heteroaryl, substituted heteroaryl, cycloalkyl, C₁ to C₆ alkyl and substituted C₁ to C₆ alkyl;
R^{2A} is absent or is selected from aryl, substituted aryl, heteroaryl, substituted heteroaryl, heterocycloalkyl, substituted heterocycloalkyl, —CH(aryl)-, —CH(substituted aryl)-, —CH(heteroaryl)- and —CH(substituted heteroaryl)-;
R^{3A} is selected from is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S;
X¹ is CH₂;
X² and X³ are each independently CH₂, or a heteroatom selected from O and NR^{x}, wherein R^{x} is H or C₁ to C₆ alkyl; and
n is 0, 1, 2, or 3; and
L shows the point of attachment of the linker;
or
   Z consists of, or consists essentially of, a structure according to formula (A1):

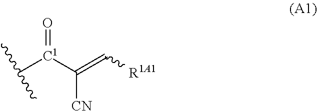

wherein:
   R^{1A} is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S; and wherein the linker is attached to carbonyl carbon C¹;
and further
wherein the BRD9 binder is of formula 1a:

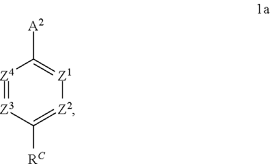

wherein:
Z¹ is N or CR^{A};
Z² is N or CR^{B};
Z³ is N or CR^{D};
Z⁴ is N or CR^{E};
wherein no more than 3 of Z¹, Z², Z³ and Z⁴ are N;
R^{A} and R^{E} are each independently selected from the group consisting of —H, —O—C₁₋₃alkyl and —C₁₋₃alkyl;
R^{B} and R^{D} are each independently selected from the group consisting of —O—C₁₋₃alkyl, —H, —OH, halogen, —NH₂, —C₁₋₃alkyl, —O—C₁₋₃haloalkyl, —C₁₋₃alkyl-O—C₁₋₃alkyl, 4-7 membered heterocycloalkyl, —C₁₋₃alkyl-SO₂—C₁₋₃alkyl, —C₁₋₃alkyl-NH₂, —C₁₋₃alkyl-N(—C₁₋₃alkyl)₂, —N(C₁₋₃alkyl)₂, —NH—R^{F};
R^{F} is selected from —SO₂—C₁₋₃alkyl and —C₁₋₃alkyl, wherein the —C₁₋₃alkyl is optionally substituted with a 5 to 6 membered heteroaryl;
alternatively, R^{A} and R^{B} taken together form a benzene ring;
alternatively, R^{C} and Z² or R^{C} and Z³ taken together (e.g. R^{C} and R^{B} or R^{C} and R^{D} taken together with the carbon atoms to which they are joined) form a 5-7 membered heterocycloalkyl optionally substituted with —C₁₋₃alkyl;
R^{C} is selected from the group consisting of —H, —Y—R^{G}, —NH₂, —C₁₋₃alkyl and 4-7 membered heterocycloalkyl;
Y is absent or is selected from the group consisting of —CR^{H}R^{I}—, —SO₂— and —CO—;
R^{H} and R^{I} are each independently selected from —H or —C₁₋₃alkyl; or R^{H} and R^{I} taken together form a —C₃₋₄cycloalkyl,
R^{G} is selected from the group consisting of —NH₂, —OH, —C₁₋₃alkyl, —N(R^{J}R^{K}), —O—R^{L}, aryl, 5-6 membered heteroaryl, wherein the aryl and heteroaryl are optionally and independently substituted with one or more halogen, optionally substituted 4- to 7-membered monocyclic heterocycloalkyl, and optionally substituted 7- to 12-membered bicyclic heterocycloalkyl, which monocylic or bicyclic heterocycloalkyl are optionally substituted with any suitable substituent, such as one or more groups independently selected from halogen, —OH, —NH₂, —C₁₋₃alkyl, —NHC₁₋₃ alkyl, —N(C₁₋₃alkyl)₂, —O—C₁₋₃alkyl and —CH₂—R^{M1};
R^{M1} is selected from 5-10 membered mono- or bicyclic aryl or heteroaryl, which is optionally substituted with —NH₂, —OH, halogen, —CN, C₁₋₃alkyl, —O—C₁₋₃alkyl;
R^{J} is —H or —C₁₋₃alkyl;
R^{K} is selected from the group consisting of —C₁₋₃alkyl, —C₂₋₃alkyl-N(C₁₋₃alkyl)₂, —C₂₋₃alkyl-NHC₁₋₃alkyl and optionally substituted 4- to 7-membered monocyclic heterocycloalkyl, and optionally substituted 7- to 12-membered bicyclic heterocycloalkyl, which monocyclic or bicyclic heterocycloalkyl are optionally substituted with any suitable substituent, such as —C₁₋₃alkyl;
R^{L} is —C₁₋₃alkyl or a 4-7 membered heterocycloalkyl, which heterocycloalkyl is optionally substituted with C₁₋₃alkyl;
wherein when R^{C} is Y—R^{G}, R^{B} and R^{D} are each independently selected from —H, —OH, halogen, —NH₂, —CN, —C₁₋₃alkyl, —C₁₋₃haloalkyl, —O—C₁₋₃alkyl, —O—C₁₋₃haloalkyl and —C₁₋₃alkyl-O—C₁₋₃alkyl;
wherein at least one of the substituents R^{A} to R^{E} is not hydrogen;
   and
A² is selected from formulae 1b or 1c:

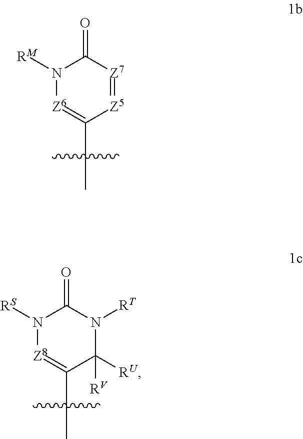

wherein the wavy lines intersect the bond between A² and the carbon atom positioned ortho to R^{A} and R^{E};
R^{M} is selected from the group consisting of optionally substituted C₁₋₆alkyl, optionally substituted C₂₋₆alkenyl, optionally substituted C₁₋₆heteroalkyl, optionally substituted C₃₋₁₀carbocyclyl, C₂₋₆alkynyl and H;
Z⁵ is N or CR^{O};
Z⁶ is N or CR^{P};
Z⁷ is N or CR^{N};
wherein only one of Z⁵, Z⁶ and Z⁷ is N;
Z⁸ is CR^{W} or N;
R^{N} is selected from the group consisting of halogen, optionally substituted —C₁₋₆alkyl, —H, C(O)C₁₋₅alkyl, —NH₂, optionally substituted amino, —OH, cyano, optionally substituted C₁₋₆heteroalkyl, optionally substituted C₃₋₁₀ carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl and thiol;
R^{O} is selected from the group consisting of H, halogen, cyano, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl, hydroxy, thiol and optionally substituted amino;
R^{P} is selected from the group consisting of H, halogen, optionally substituted C₁₋₆alkyl, optionally substituted C₁₋₆heteroalkyl, optionally substituted C₃₋₁₀carbocyclyl and optionally substituted C₆₋₁₀aryl;
alternatively, R^{N} and Z⁵ taken together, combine to form an optionally substituted C₆₋₁₀arene or optionally substituted C₂₋₉heteroarene; optionally wherein R^{N} and R^{O} taken together with the carbon atoms to which they are joined, combine to form an optionally substituted C₆₋₁₀arene or optionally substituted C₂₋₉heteroarene;
R^{S} is selected from the group consisting of H, optionally substituted C₁₋₆alkyl, optionally substituted C₁₋₆heteroalkyl and optionally substituted C₃₋₁₀carbocyclyl;
R^{T} is selected from the group consisting of H, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl, optionally substituted sulfone and optionally substituted sulfonamide, or R^{T} and R^{U} together with the atoms to which each is attached, form an optionally substituted C₂₋₉heterocyclyl;
R^{U} and R^{V} are each independently selected from the group consisting of H, halogen, hydroxyl, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl, thiol, optionally substituted sulfone and optionally substituted amino;
alternatively, R^{T} and R^{U} together with the atoms to which each is attached, form an optionally substituted C₂₋₉heterocyclyl;
R^{W} is selected from the group consisting of H, halogen, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl and optionally substituted C₂₋₉heteroaryl;
and
wherein the BRD9 binder is attached to the linker at any suitable position.

2. The bifunctional molecule of clause 1, wherein up to 1 of Z¹, Z², Z³ and Z⁴ is N.

3. The bifunctional molecule of clause 1 or clause 2, wherein the BRD9 binder is of formula 1a′:

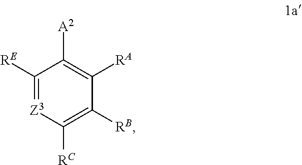

wherein:
R^{A}, R^{B}, R^{C}, R^{E}, Z³ and A² are as defined in clause 1 or 2.

4. The bifunctional molecule of any one of clause 1 to 3, wherein A² is selected from formula 1b′, wherein formula 1b′ is:

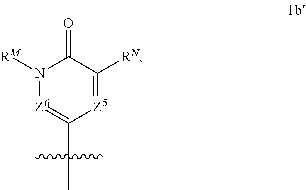

wherein the wavy line intersects the bond between A² and the carbon atom positioned ortho to R^{A} and R^{E};
R^{M} is selected from the group consisting of —C₁₋₅alkyl, -cyclopropyl, —C₁₋₄haloalkyl and H;
R^{N} is selected from the group consisting of halogen, —C₁₋₅alkyl, —C₁₋₃haloalkyl, —H, C(O)C₁₋₅alkyl, —NH₂, —NHC₁₋₃alkyl and —OH;
Z⁵ is N or CR^{O}
Z⁶ is N or CR^{P} wherein only one of Z⁵ and Z⁶ may be N;
R^{O} is H or —C₁₋₃alkyl;
R^{P} is H or —C₁₋₃alkyl;
wherein only one of R^{O} and R^{P} may be —C₁₋₃alkyl;
alternatively, R^{N} and Z⁵ taken together form a benzene ring or a 5-6 membered heteroarene ring, each of which rings can be optionally and independently substituted with one or more groups selected from halogen, —OH, —NH₂, —NH—C₁₋₃alkyl and —C₁₋₅alkyl, C₁₋₅haloalkyl, C₁₋₅alkoxy, C₁₋₄haloalkoxy, Formula 1d (shown below), C₃₋₅azacycloalkyl, C₂₋₅alkenyl, C₂₋₅alkynyl, C₃₋₅cycloalkyl, wherein the —C₁₋₅alkyl group can be optionally substituted with 5-6 membered heteroaryl or phenyl;

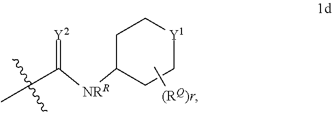

wherein
Y² is NR^{R} or O;
Y¹ is S(O)ₐ or NR^{R};
each R^{R} is independently H or C₁₋₄alkyl;
each R^{Q} is independently selected from the group consisting of C₁₋₄alkyl, C₁₋₄haloalkyl, halogen and —C(O)C₁₋₃alkyl;
a is 0 to 2; and
r is 0 to 3.

5. The bifunctional molecule of any one of clauses 1 to 4, wherein the BRD9 binder is of formula 1e, 1f or 1g:

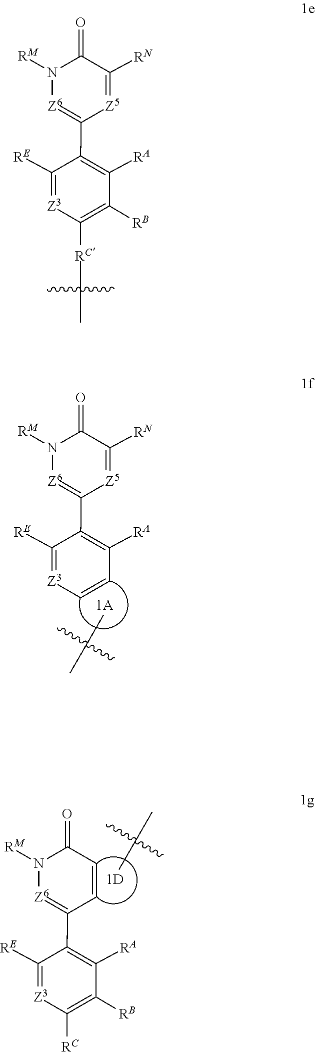

wherein the wavy line intersects the bond between the BRD9 binder and the linker; wherein R^{A}, R^{B}, R^{C}, R^{E}, R^{M}, R^{N}, Z³, Z⁵ and Z⁶ are as defined in any one of clauses 1 to 4;
wherein R^{C′} is absent, or is as defined for R^{C} in any one of clauses 1 to 4;
ring 1A is a 5-7 membered heterocycloalkane optionally substituted with —C₁₋₃alkyl; and
ring 1D is an optionally substituted C₆₋₁₀arene or optionally substituted C₂₋₉heteroarene.

6. The bifunctional molecule of clause 5, wherein ring 1A comprises one or two heteroatoms independently selected from the list consisting of N, S and O.

7. The bifunctional molecule of clause 5 wherein ring 1A is selected from the list consisting of pyrrolidine, piperidine, piperazine, morpholine, oxolane, oxane, tetrahydrothiophene and thiane.

8. The bifunctional molecule of any one of clauses 1 to 5, wherein the BRD9 binder is of formula 1e, 1f′ or 1g′:

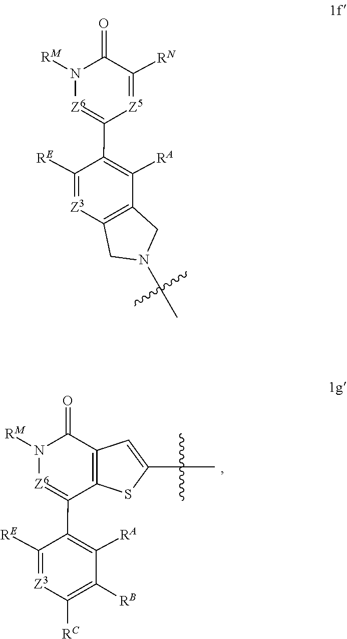

wherein the wavy line intersects the bond between the BRD9 binder and the linker; and wherein R^{A}, R^{B}, R^{C}, R^{E}, R^{M}, R^{N}, Z³, Z⁵ and Z⁶ are as defined in any one of clauses 1 to 4.

9. The bifunctional molecule of any one of clauses 1 to 8, wherein R^{A}, R^{B}, R^{C}, R^{D} and R^{E} are independently selected from —O—C₁₋₃alkyl, —H, halogen, —O—C₁₋₃haloalkyl, —OH, —NH₂, —C₁₋₃alkyl, —C₁₋₃alkyl-NH₂, —C₁₋₃alkyl-N(—C₁₋₃alkyl)₂ and —N(C₁₋₃alkyl)₂.

10. The bifunctional molecule of any one of clauses 1 to 9, wherein at least two of R^{A}, R^{B}, R^{D} and R^{E} are —H.

11. The bifunctional molecule of any one of clauses 1 to 10, wherein at least one of R^{A}, R^{B}, R^{D} and R^{E} is selected from the group consisting of —O—C₁₋₃alkyl, —H, halogen and —O—C₁₋₃haloalkyl.

12. The bifunctional molecule of any one of clauses 1 to 11, wherein R^{M} is —C₁₋₅alkyl.

13. The bifunctional molecule of any one of clauses 1 to 12, wherein R^{N} is —C₁₋₅alkyl or halogen, or R^{N} and Z⁵ taken together form an optionally substituted 5-6 membered heteroarene or benzene ring.

14. The bifunctional molecule of clause 13, wherein the optionally substituted 5-6 membered heteroarene ring comprises one or more heteroatoms selected from the group consisting of N, S and O.

15. The bifunctional molecule of clause 13, wherein the optionally substituted 5-6 membered heteroarene ring is an N- or S-heteroarene.

16. The bifunctional molecule of clause 13, wherein the optionally substituted 5-6 membered heteroarene ring is any one selected from the optionally substituted group consisting of pyridine, pyrrole, imidazole, pyrimidine, thiophene and pyrazole.

17. The bifunctional molecule of any one of clauses 1 to 16, wherein the BRD9 binder is any one of formulae 1ea to 1eh and 1fa to 1ﬁ and 1ga:

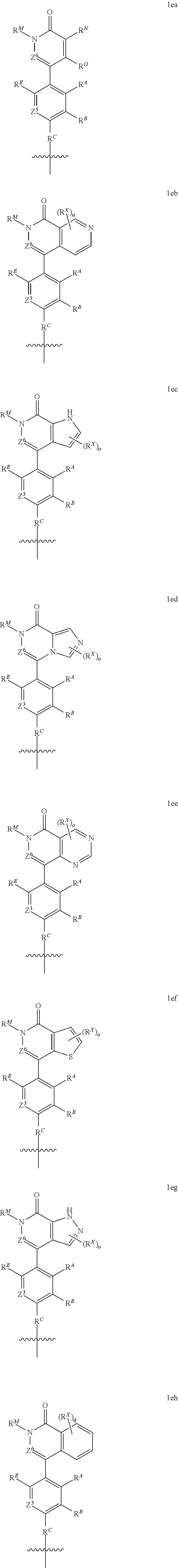

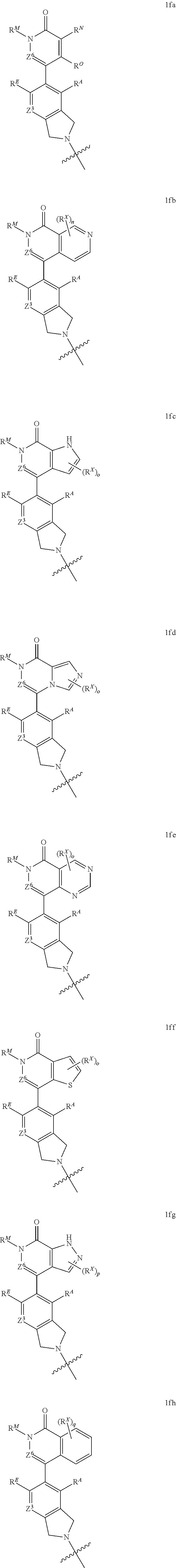

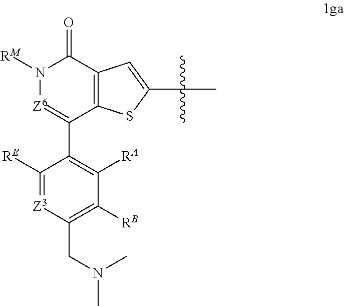

wherein the wavy line intersects the bond between the BRD9 binder and the linker;
R^{A}, R^{B}, R^{E}, R^{M}, Z³ and Z⁶ are as defined in any one of clauses 1 to 11;
R^{C} is absent, or is as defined in any one of clauses 1 to 11;
R^{N} is selected from the group consisting of halogen, —C₁₋₅alkyl, —C₁₋₃haloalkyl, —H, C(O)C₁₋₅alkyl, —NH₂, —NHC₁₋₃alkyl and —OH;
R^{O} is H or —C₁₋₃alkyl;
each R^{X} is independently selected from the group consisting of halogen, —OH, —NH₂, —NH—C₁₋₃alkyl —C₁₋₅alkyl, C₁₋₅haloalkyl, C₁₋₅alkoxy and C₁₋₄haloalkoxy;
n is 0 to 3;
is 0 to 2;
p is 0 or 1; and
q is 0 to 4.

18. The bifunctional molecule of any one of clauses 1 to 17, wherein the BRD9 binder is according to formula 1ea′:

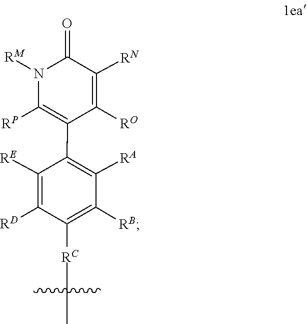

wherein the wavy line intersects the bond between the BRD9 binder and the linker;
R^{A} and R^{E} are each independently selected from H and —O—C₁₋₃alkyl;
R^{B} and R^{D} are each independently selected from —O—C₁₋₃alkyl, —H, -halo, —C₁₋₃alkyl, and —O—C₁₋₃haloalkyl;
R^{C} is absent, or is —Y—R^{G};
Y is selected from the group consisting of —CR^{H}R^{I}—, and —CO—;
R^{H} and R^{I} are each independently selected from —H or —C₁₋₃alkyl; or R^{H} and R^{I} taken together form a —C₃₋₄cycloalkyl;
R^{G} is selected from the group consisting of —N(R^{J}R^{K}) (e.g. —N(C₁₋₃alkyl)-, —N(C₁₋₃alkyl)(optionally substituted 4- to 7-membered monocyclic heterocycloalkylene), or —N(C₁₋₃alkyl(optionally substituted 7- to 12-membered bicyclic heterocycloalkylene)); —O—; optionally substituted 4- to 7-membered monocyclic heterocycloalkylene; and optionally substituted 7- to 12-membered bicyclic heterocycloalkylene;
R^{J} and R^{K} are as defined in clause 1; —
R^{M} is C₁₋₃alkyl; and
R^{N}, R^{O} and R^{P} are each independently selected from the group consisting of halo, —C₁₋₃alkyl, and —C₁₋₃haloalkyl.

19. The bifunctional molecule of any one of clauses 1 to 18, wherein the BRD9 binder is any one of formulae 1h to 1z and 2a to 2g:

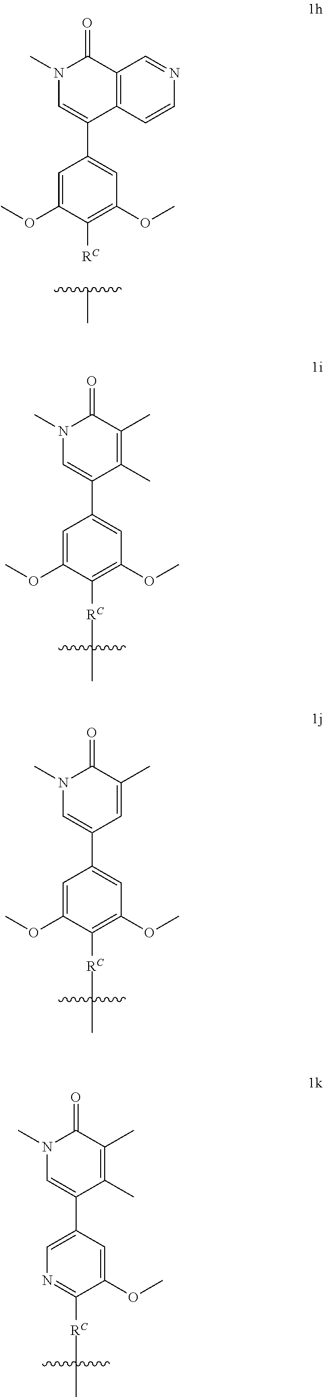

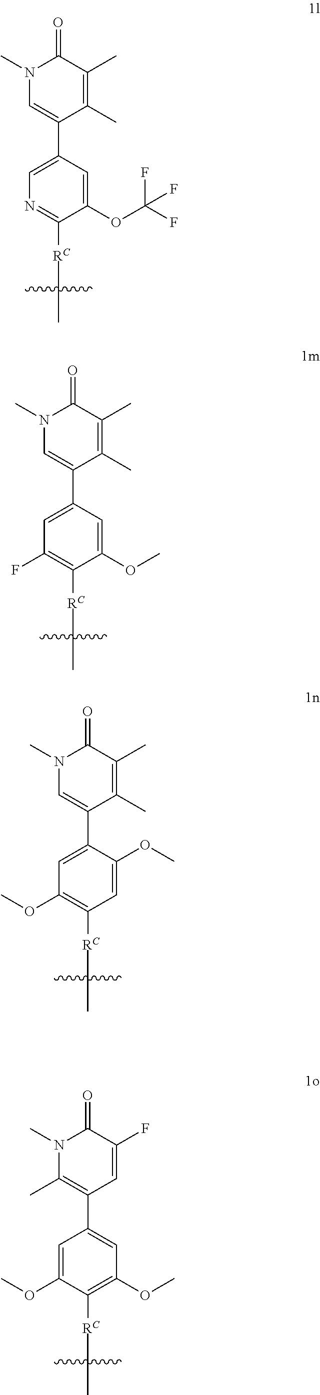

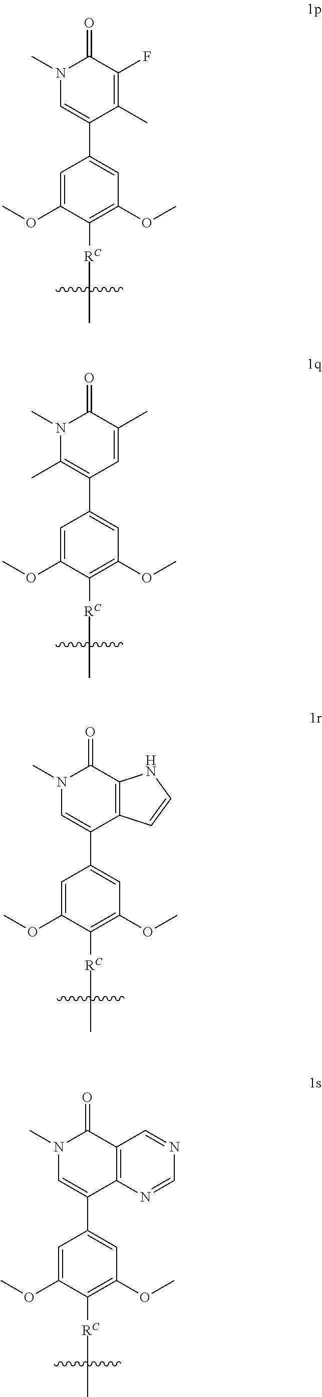

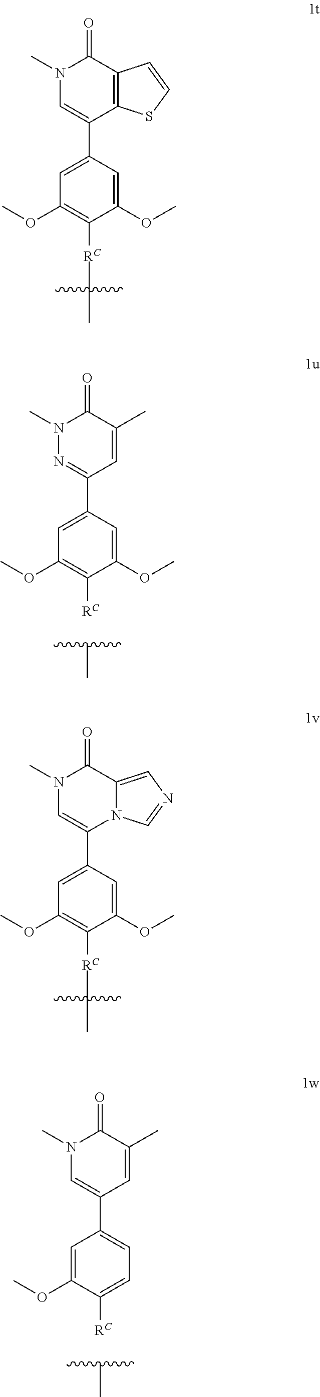

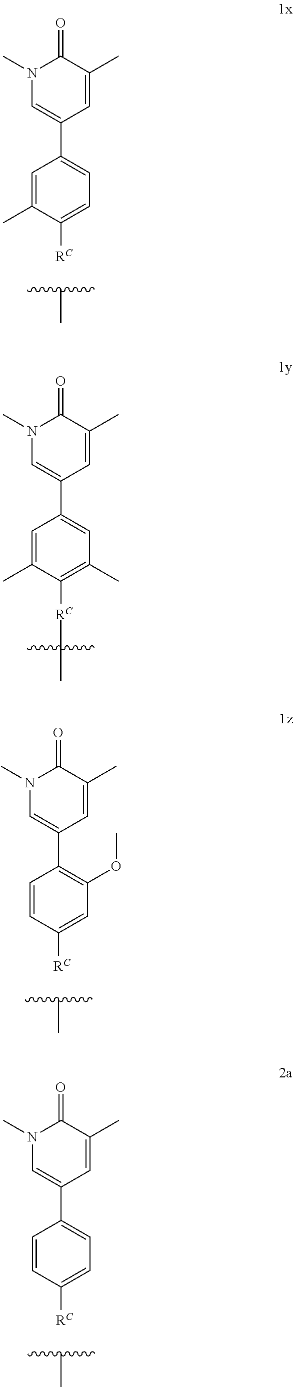

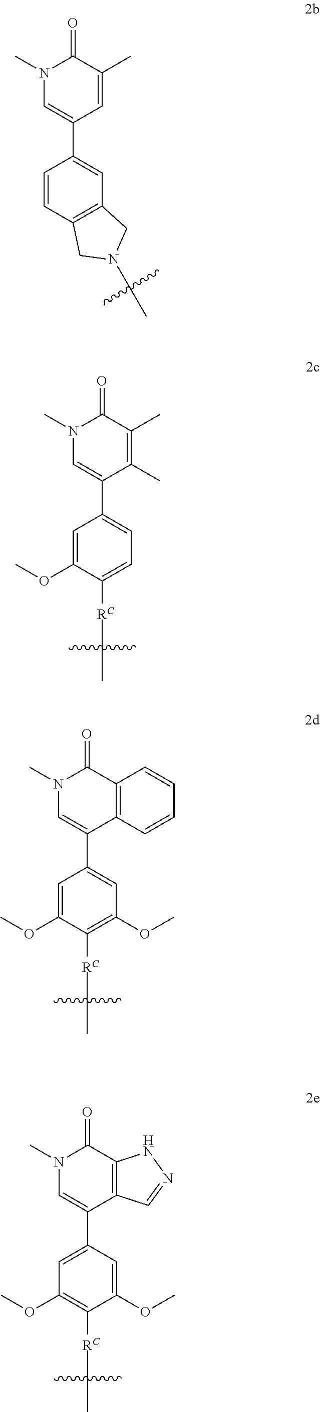

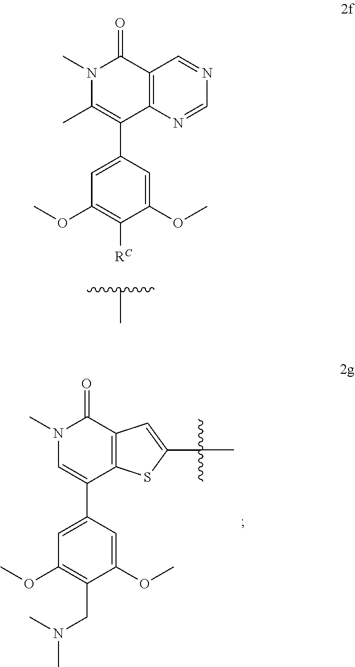

wherein R^{C} is absent, or is —Y—R^{G};
Y is selected from the group consisting of —CR^{H}R^{I}—, and —CO—;
R^{H} and R^{I} are each —H; or R^{H} and R^{I} taken together form a —C₃₋₄cycloalkyl;
R^{G} is selected from the group consisting of —N(R^{J}R^{K}) (e.g. —N(C₁₋₃alkyl)-, N(C₁₋₃alkyl)(optionally substituted 4- to 7-membered monocyclic heterocycloalkylene), or —N(C₁₋₃alkyl)(optionally substituted 7- to 12-membered bicyclic heterocycloalkylene)); —O—; optionally substituted 4- to 7-membered monocyclic heterocycloalkylene containing one or two N ring atoms; and optionally substituted 7- to 12-membered bicyclic heterocycloalkylene containing one or two N ring atoms;
R^{J} and R^{K} are as defined in clause 1;
wherein the wavy line intersects the bond between the BRD9 binder and the linker.

20. A bifunctional molecule according to any one of clauses 1 to 19, wherein R^{C} is present and is any one selected from:

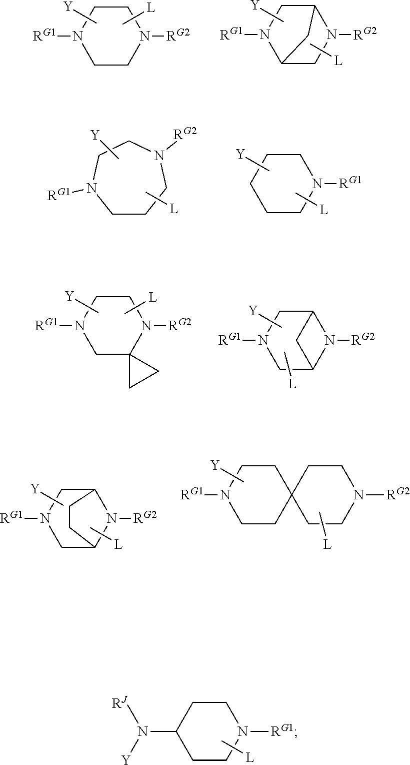

wherein Y is CR^{H}R^{I} (e.g. CH₂);
R^{G1} and R^{G2} are each independently selected from H and C₁-C₃ alkyl;
R^{J} is as defined in claim 1; and
L shows the point of attachment of the linker.

21. A bifunctional molecule according to any one of clauses 1 to 20, wherein:
(i) when R¹ and R⁴ together form a 5-, 6-, or 7-membered heterocyclic ring, Z is represented by formula (Ia):

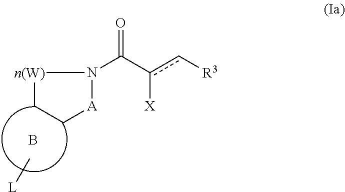

wherein A, B, R³ and L are as defined for formula (I); and
n is 1, 2 or 3;
W is selected from CR^{W1}R^{W2}, O, NR^{W3} and S;
R^{W1}, R^{W2} and R^{W3} are each independently selected from H and C₁ to C₆ alkyl; and wherein when n is 2 or 3, each W is independently selected from CR^{W1}R^{W2}, O, NR^{W3}, and S;
(ii) when R¹ and R² together form a 5-, 6-, or 7-membered heterocyclic ring, Z is represented as formula (Ib):

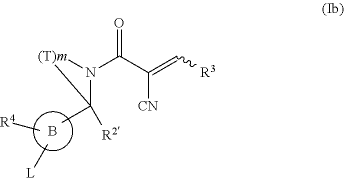

Wherein B, R^{2′}, R³, R⁴ and L are as defined for formula (I);
m is 3, 4 or 5;
each T is independently selected from CR^{T1}R^{T2}, O, NR^{T3} and S; and
R^{T1}, R^{T2} and R^{T3} are each independently selected from H and C₁ to C₆ alkyl; or
(iii) when R² and R⁴ together form a 5-, 6-, or 7-membered heterocyclic or carbocyclic ring, Z is represented as formula (Ic):

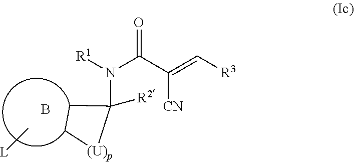

Wherein B, R¹, R^{2′}, R³ and L are as defined for formula (I);
p is 2, 3 or 4; and
each U is independently selected from CR^{U1}R^{U2}, O, NR^{U3} and S; and
R^{U1}, R^{U2} and R^{U3} are each independently selected from H and C₁ to C₆ alkyl.

22. The bifunctional molecule according to any one of the preceding clauses, wherein R³ is selected from the group consisting of a heteroaryl, substituted heteroaryl, C₁-C₆ alkyl, C₃-C₆ cycloalkyl, C₁-C₆ cycloheteroalkyl, C₁-C₆ alkyl substituted with a heterocyclic group, aryl, and substituted aryl,
optionally wherein R³ is selected from:

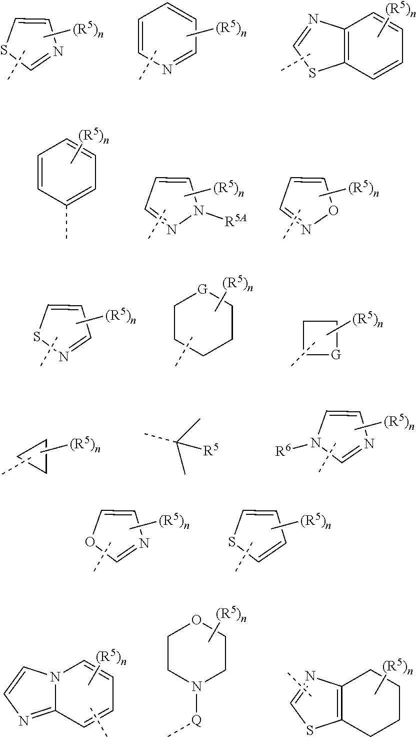

wherein the dotted line indicates the position at which each of the respective R³ groups is joined to the structure shown in formula (I) to (Ic), or wherein when the dotted line is not appended to an atom, the dotted line indicates that each of the respective R³ groups is joined to the structure via any position on the aromatic or heteroaromatic ring;
each R⁵ is independently selected from the group consisting of halo, CF₃, —CH₂F, —CHF₂, —OCF₃, —OCH₂F, —OCHF₂, C₁ to C₆ alkyl, —CN, —OH, —OMe, —SMe, —SOMe, —SO₂Me, —NH₂, —NHMe, —NMe₂, CO₂Me, —NO₂, CHO and COMe;
n is 0 to 3;
R⁶ is C₁ to C₆ alkyl;
G is CH₂, O and NH; and
Q is C₁ to C₆ alkylene.

23. The bifunctional molecule according to any one of the preceding clauses, wherein A is CR²R^{2′}, optionally wherein: (i) one of R² and R^{2′} is a hydrogen and the other is C₁ to C₆ alkyl, optionally wherein the C₁ to C₆ alkyl is substituted with one or more halo atoms; or (ii) both of R² and R^{2′} are selected from C₁ to C₆ alkyl.

24. The bifunctional molecule according to any one of the preceding clauses, wherein B is a phenyl group.

25. The bifunctional molecule according to any one of the preceding clauses, wherein Z is represented as formula (IIaa):

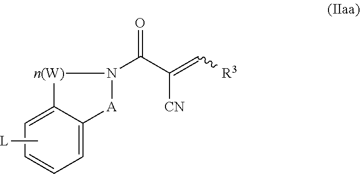

wherein A, R³, and L are as defined for formula (I);
n is 1, 2 or 3; and
W is selected from CR^{W1}R^{W2}, O, NR^{W3} and S; and
R^{W1}, R^{W2} and R^{W3} are each independently selected from H and C₁ to C₆ alkyl; and
wherein when n is 2 or 3, each W is independently selected from CR^{W1}R^{W2}, O, NR^{W3}, and S.

26. The bifunctional molecule according to any one of the preceding clauses, wherein Z is represented as formula (IIa):

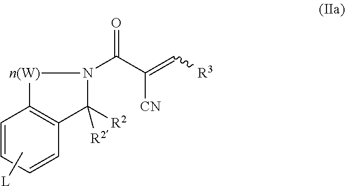

wherein R², R^{2′}, R³ and L are as defined in any one of the preceding clauses,
n is 1, 2 or 3; and
W is selected from CR^{W1}R^{W2}, O, NR^{W3} and S; and
R^{W1}, R^{W2} and R^{W3} are each independently selected from H and C₁ to C₆ alkyl; and
wherein when n is 2 or 3, each W is independently selected from CR^{W1}R^{W2}, O, NR^{W3}, and S.

27. The bifunctional molecule according to any one of the preceding clauses, wherein Z is represented as formula (IIb)

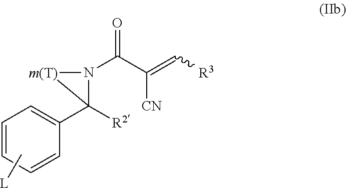

wherein R^{2′}, R³ and L are as defined in any one of the preceding clauses;
m is 3, 4 or 5; and
each T is independently selected from CR^{T1}R^{T2}, O, NR^{T3} and S; and
R^{T1}, R^{T2} and R^{T3} are each independently selected from H and C₁ to C₆ alkyl.

28. The bifunctional molecule of any one of clauses 1 to 25, wherein Z is represented as formula (Ia) or (IIaa).

29. The bifunctional molecule of any one of clauses 1 to 20, wherein Z is represented by formula (WZIa):

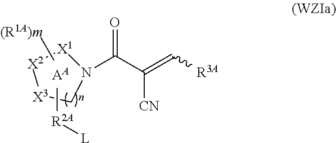

wherein:
R^{1A} is absent (i.e. when m is 0) or is selected from aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R^{1A} groups combine to form an optionally substituted C₁₋₃ bridge, optionally substituted C₃₋₅cycloalkyl or optionally substituted 5- to 7-membered heterocycloalkyl (e.g. 5- to 7-membered N-heterocycloalkyl), optionally wherein the C₃₋₅cycloalkyl or the 5- to 7-membered heterocycloalkyl are joined to ring A^{A} at a spiro centre; R^{2A} is absent or is selected from aryl, substituted aryl, heteroaryl, substituted heteroaryl, heterocycloalkyl, substituted heterocycloalkyl, NR^{y}, —CH(aryl)-, —CH(substituted aryl)-, —CH(heteroaryl)- and —CH(substituted heteroaryl)-;
wherein R^{y} is optionally substituted C₁₋₆alkyl or H;
R^{3A} is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S;
X¹ is CH₂;
X², X³ and X⁴ are each independently CH₂, O or NR^{x};
R^{x} is H or C₁ to C₆ alkyl, or wherein one R^{1A} group and one R^{x} group combine to form an optionally substituted C₁₋₃ bridge;
n is 0, 1, 2, or 3;
m is 0, 1, 2, 3 or 4; and
L shows the point of attachment of the linker.

30. The bifunctional molecule of any one of clauses 1 to 20, wherein Z is represented by formula (WZII):

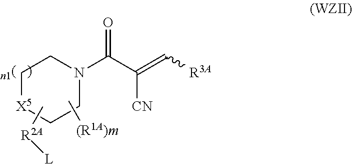

wherein R^{2A} is absent or is as described in clause 1 or 29;
R^{3A} is as described in clause 1 or 29;
X⁵ is CR^{b}₂, NR^{b}, O or a 5- to 7-membered heterocycloalkyl (e.g. a 5- to 7-membered heterocycloalkyl);
each R^{1A} is independently selected from aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R^{1A} groups combine to form an optionally substituted C₁₋₃ bridge or optionally substituted C₃₋₅cycloalkyl (optionally wherein the C₃₋₅cycloalkyl is joined to the heterocyclic ring shown in formula (WZII) at a spiro centre);
R^{b} is H or optionally substituted C₁₋₃alkyl;
n1 is 0, 1, 2 or 3;
m is 0, 1 or 2; and
L shows the point of attachment of the linker.

31. The bifunctional molecule of any one of clauses 1 to 20, wherein Z is represented by any one of formulae (WZIIa) to (WZIIe):

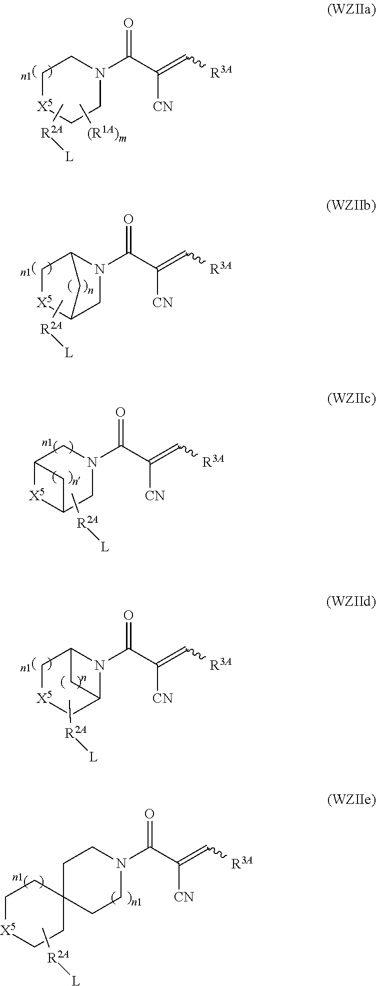

wherein:
R^{2A} is as defined in clause 1 or 29;
R^{3A} is as defined in clause 1 or 29;
each R^{1A} is independently selected from aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R^{1A} groups combine to form an optionally substituted C₃₋₅cycloalkyl (optionally wherein the C₃₋₅cycloalkyl is joined to the heterocyclic ring shown in formula (ZIIa) at a spiro centre);
X⁵ is C(R^{b})₂, NR^{b} or O;
R^{b} is H or optionally substituted C₁₋₃alkyl;
n1 is 0, 1, 2 or 3;
n′ is 1 or 2;
m is 0, 1 or 2; and
L shows the point of attachment of the linker.

32. The bifunctional molecule of any one of clauses 1 to 20, wherein Z is represented by formula (WII):

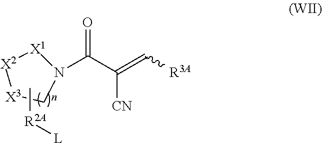

wherein R^{2A} is selected from aryl, substituted aryl, heteroaryl, substituted heteroaryl, heterocycloalkyl, substituted heterocycloalkyl, —CH(aryl)-, —CH(substituted aryl)-, —CH(heteroaryl)- and —CH(substituted heteroaryl);
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group;
X¹ is CH₂;
X² and X³ are each independently CH₂ or O; with the proviso that none or only 1 of X² and X³ is O; and
n is 0, 1, 2 or 3; and
L shows the point of attachment of the linker.

33. The bifunctional molecule of any one of clauses 1 to 19, wherein Z is represented by formula (WIIa):

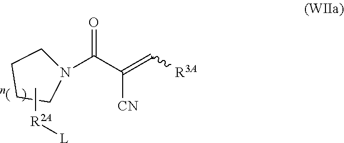

wherein R^{2A} is selected from aryl, substituted aryl, heteroaryl, substituted heteroaryl, heterocycloalkyl, substituted heterocycloalkyl, —CH(aryl)-, —CH(substituted aryl)-, —CH(heteroaryl)- and —CH(substituted heteroaryl);
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group; and
n is 0, 1, 2 or 3; and
L shows the point of attachment of the linker.

34. The bifunctional molecule of any one of clauses 1 to 19, wherein Z is represented by formula (WIIb):

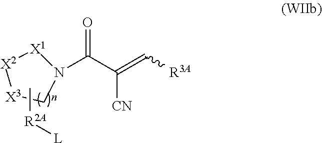

wherein R^{2A} is selected from aryl substituted aryl, heteroaryl, substituted heteroaryl, heterocycloalkyl, and substituted heterocycloalkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group;
X¹ is CH₂;
X² and X³ are each independently CH₂ or O; with the proviso that none or only 1 of X² and X³ is O;
n is 1 or 2; and
L shows the point of attachment of the linker.

35. The bifunctional molecule of any one of clauses 1 to 19, wherein Z is represented by formula (WIIc):

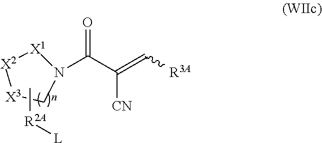

wherein R^{2A} is selected from heterocycloalkyl and substituted heterocycloalkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group;
X¹ is CH₂;
X² and X³ are each independently CH₂ or O; with the proviso that none or only 1 of X² and X³ is O;
n is 1 or 2; and
L shows the point of attachment of the linker.

36. The bifunctional molecule of any one of clauses 1 to 19, wherein Z is represented by formula (WIId):

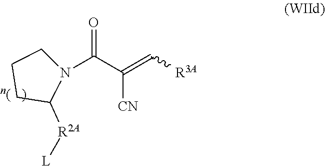

wherein R^{2A} is selected from heterocycloalkyl and substituted heterocycloalkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group;
n is 1 or 2; and
L shows the point of attachment of the linker.

37. The bifunctional molecule of any one of clauses 1 to 19, wherein Z is represented by formula (WIIe):

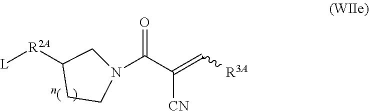

wherein R^{2A} is selected from aryl, substituted aryl, heteroaryl, substituted heteroaryl, heterocycloalkyl and substituted heterocycloalkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group;
n is 1 or 2; and
L shows the point of attachment of the linker.

38. The bifunctional molecule of any one of clauses 1 to 19, wherein Z is represented by formula (WIIf):

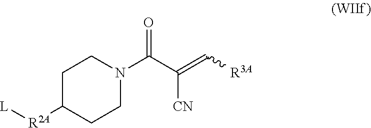

wherein R^{2A} is selected from aryl, substituted aryl, heteroaryl, substituted heteroaryl, heterocycloalkyl and substituted heterocycloalkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group; and
L shows the point of attachment of the linker.

39. The bifunctional molecule of any one of clauses 1 to 19, wherein Z is represented by formula (WIII):

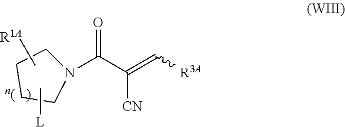

wherein R^{1A} is selected from aryl, substituted aryl, heteroaryl, substituted heteroaryl, cycloalkyl and C₁ to C₆ alkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group; and
n is 0, 1, 2 or 3; and
L shows the point of attachment of the linker.

40. The bifunctional molecule of any one of clauses 1 to 19, wherein Z is represented by formula (WIIIa):

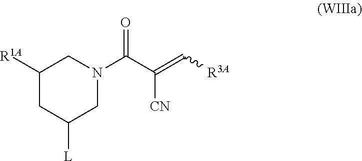

wherein R^{1A} is selected from aryl, substituted aryl, heteroaryl, substituted heteroaryl, cycloalkyl and C₁ to C₆ alkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group; and
L shows the point of attachment of the linker.

41. The bifunctional molecule of any one of clauses 1 to 19, wherein Z is represented by formula (WIIIb):

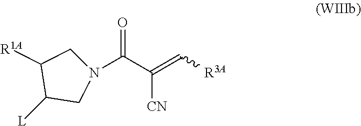

wherein R^{1A} is selected from aryl, substituted aryl, heteroaryl, substituted heteroaryl, cycloalkyl and C₁-C₆ alkyl;
R^{3A} is selected from C₁ to C₆ alkyl, aryl, heteroaryl, substituted aryl, and substituted heteroaryl, optionally wherein the C₁ to C₆ alkyl is substituted with a heterocycloalkyl group; and
L shows the point of attachment of the linker.

42. The bifunctional molecule of any one of clauses 1 to 19, wherein Z is represented by formula (WIV):

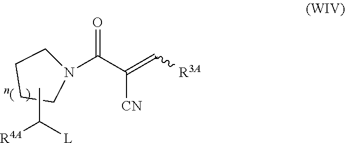

wherein R^{3A} is selected from C₁ to C₆ alkyl, aryl, heteroaryl, substituted aryl, and substituted heteroaryl, optionally wherein the C₁ to C₆ alkyl is substituted with a heterocycloalkyl group;
R^{4A} is selected from aryl, substituted aryl, heteroaryl and substituted heteroaryl; and
n is 0, 1, 2 or 3; and
L shows the point of attachment of the linker.

43. The bifunctional molecule of any one of clauses 1 to 19, wherein Z is represented by formula (WIVa):

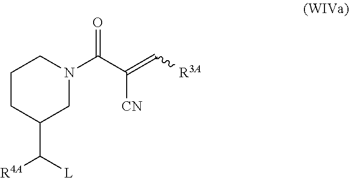

wherein R^{3A} is selected from C₁ to C₆ alkyl, aryl, heteroaryl, substituted aryl, and substituted heteroaryl, optionally wherein the C₁ to C₆ alkyl is substituted with a heterocycloalkyl group;
R^{4A} is selected from aryl, substituted aryl, heteroaryl and substituted heteroaryl; and
L shows the point of attachment of the linker.

44. The bifunctional molecule of any one preceding clause, wherein n is 1 or 2.

45. The bifunctional molecule of any one of clauses 35 to 37, wherein R^{1A} is:
(i) selected from the group consisting of phenyl that is optionally substituted with one to three substituents selected from the group consisting of halo, C₁ to C₆ alkyl, C₁ to C₆ haloalkyl and C₁ to C₆ alkoxy; and heteroaryl having 5 to 6 ring atoms containing 1 to 3 heteroatoms each independently selected from N, O and S, the heteroaryl being optionally substituted with one to three substituents selected from the group consisting of halo, C₁ to C₆ alkyl, C₁ to C₆ haloalkyl and C₁ to C₆ alkoxy; C₃ to C₈ cycloalkyl; or
(ii) selected from the group consisting of phenyl, substituted phenyl, pyrazolyl, and substituted pyrazolyl.

46. The bifunctional molecule of any one clauses 35 to 37, wherein R^{1A} is a C₃ to C₇ cycloalkyl, or a C₁ to C₃ alkyl.

47. The bifunctional molecule of any one clauses 35 to 37, wherein R^{1A} is selected from any one of the following structures:

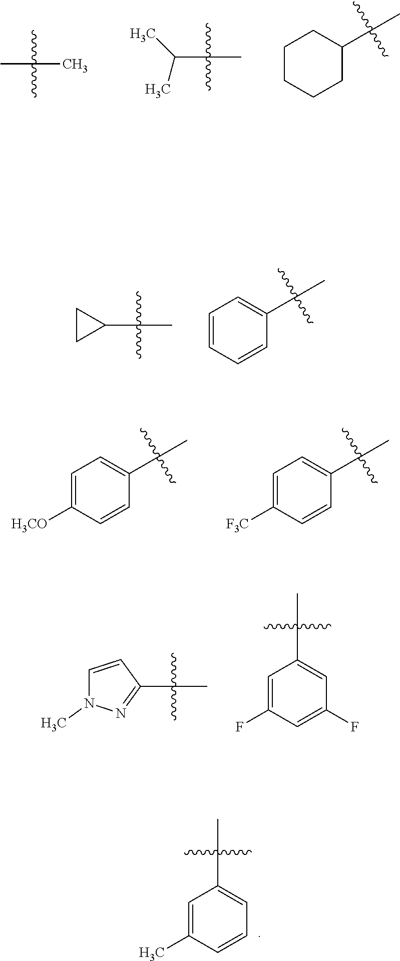

48. The bifunctional molecule of any one clauses 28 to 34, wherein R^{2A} is:
(i) selected from phenyl optionally substituted with one to three substituents selected from H, C₁ to C₆ alkyl, halo, C₁ to C₆ haloalkyl and C₁ to C₆ alkoxy; and heteroaryl having 5 to 6 ring atoms and containing 1 or 2 N atoms, the heteroaryl being optionally substituted with one to three substituents selected from C₁-C₆ alkyl, halo, C₁-C₆ haloalkyl and C₁ to C₆ alkoxy;
(ii) selected from optionally substituted phenyl, and optionally substituted pyrazolyl; or
(iii) selected from one of the following structures:

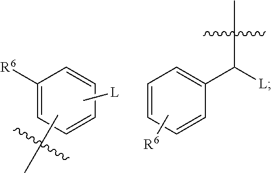

wherein R⁶ is selected from H, C₁-C₆ alkyl, halo, C₁-C₆ haloalkyl and C₁-C₆ alkoxy.

49. The bifunctional molecule of any one of clauses 28 to 34, wherein R^{2A} is:
(i) an optionally substituted heterocycloalkyl, wherein the heterocycloalkyl has 3 to 10 ring atoms and contains 1 to 3 heteroatoms each independently selected from N, O and S;
(ii) selected from optionally substituted piperidinyl, and optionally substituted piperazinyl; or
(iii) selected from one of the following structures:

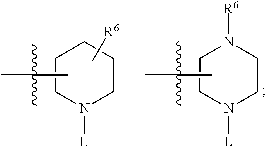

wherein R⁶ is selected from H, C₁ to C₆ alkyl, halo, C₁ to C₆ haloalkyl and C₁ to C₆ alkoxy.

50. The bifunctional molecule of any one of clauses 28 to 45, wherein R^{3A} is:
(i) selected from C₁ to C₆ alkyl optionally substituted with a heterocycloalkyl group having 5 to 7 ring atoms and containing 1 or 2 heteroatoms each independently selected from N, O and S; aryl having 6 to 10 carbon ring atoms; and heteroaryl having 5 to 10 ring atoms and containing 1 to 3 heteroatoms each independently selected from N, O and S; wherein the aryl and the heteroaryl are optionally substituted with one or two substituents selected from the group consisting of halo, C₁ to C₃ alkyl, C₁ to C₃ haloalkyl and C₁ to C₃ alkoxy; or
(ii) selected from optionally substituted phenyl, optionally substituted thiazolyl, optionally substituted pyrazolyl, optionally substituted oxazoyl, tert-butyl, C₁-C₆ alkyl comprising a morpholino substituent, optionally substituted benzothiazolyl and optionally substituted pyridinyl.

51. The bifunctional molecule of any one of clauses 28 to 45, wherein R^{3A} is selected from one of the following structures:

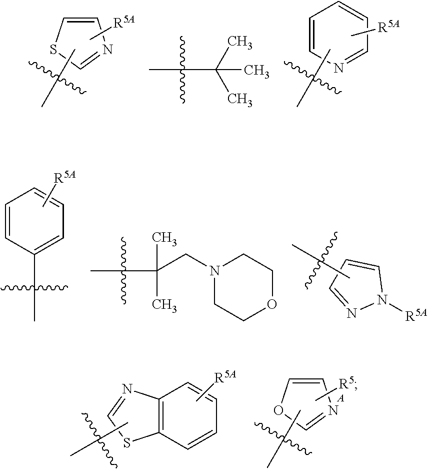

wherein R^{5A} is absent or is selected from halo (e.g. F, Cl, Br, I), CF₃, —CH₂F, —CHF₂, —OCF₃, —OCH₂F, —OCHF₂, C₁ to C₆ alkyl, —CN, —OH, —OMe, —SMe, —SOMe, —SO₂Me, —NH₂, —NHMe, —NMe₂, CO₂Me, —NO₂, CHO, and COMe.

52. The bifunctional molecule of any one of clauses 28 to 45, wherein R^{3A} is selected from one of the following structures:

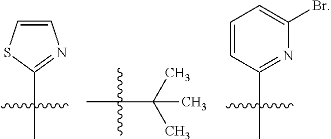

53. The bifunctional molecule of any one of clauses 38 to 40 and 46 to 48, wherein R^{4A} is:
(i) selected from aryl having 6 to 10 carbon ring atoms; and heteroaryl having 5 to 10 ring atoms and containing 1 to 3 heteroatoms each independently selected from N, O and S; wherein the aryl and the heteroaryl are optionally substituted with one or two substituents selected from the group consisting of halo, C₁ to C₃ alkyl, C₁ to C₃ haloalkyl and C₁ to C₃ alkoxy; or
(ii) optionally substituted phenyl.

54. The bifunctional molecule of clause 1, wherein Z comprises one of the following structures:

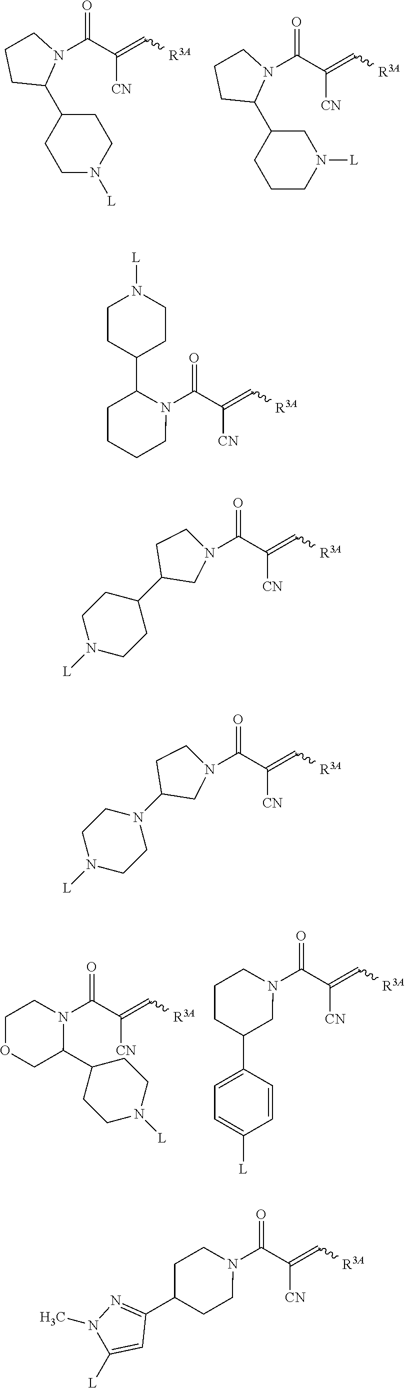

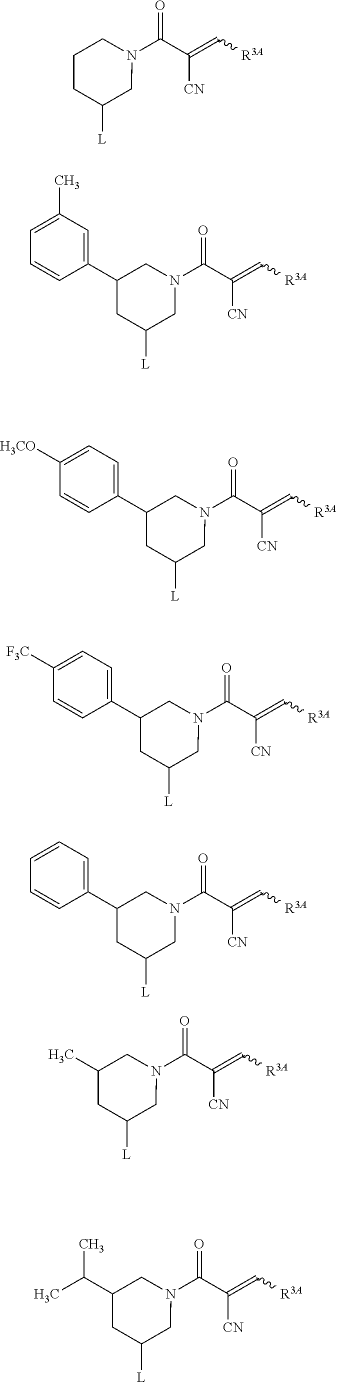

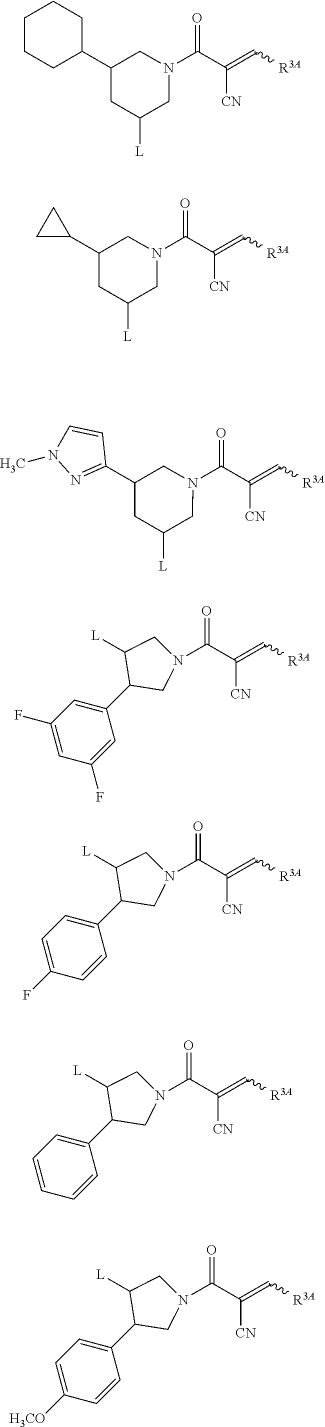

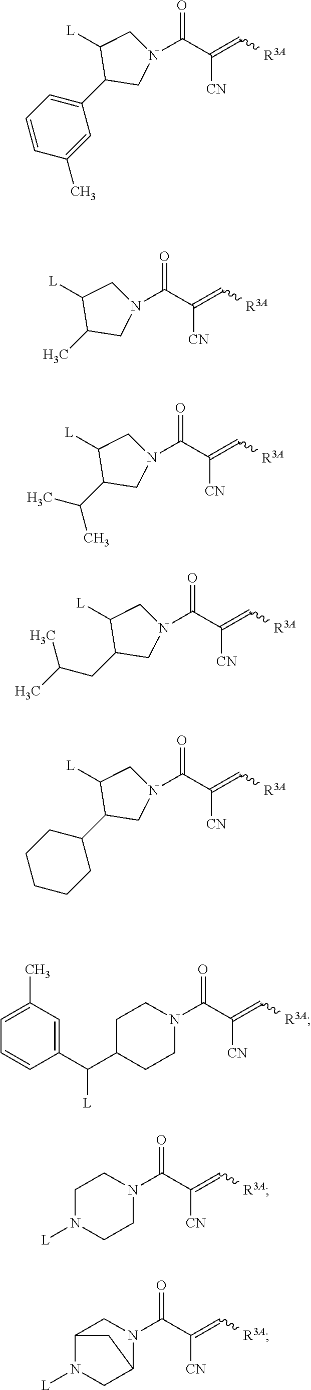

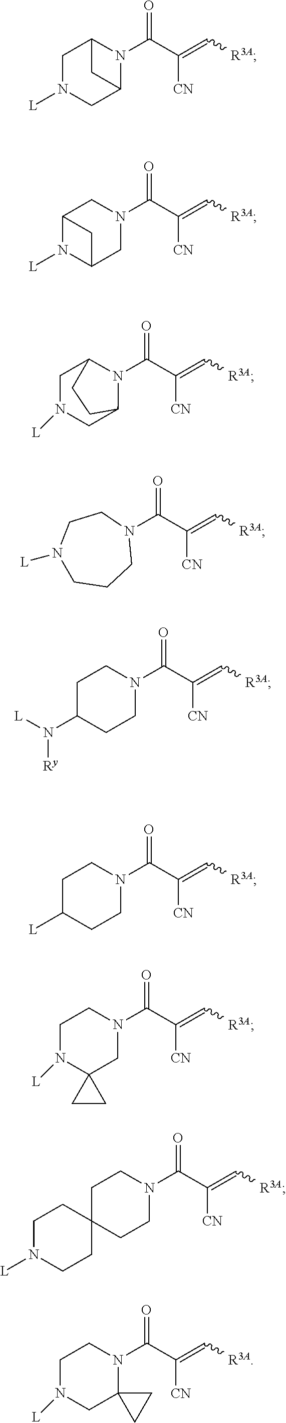

wherein R^{3A} in each of the structures above is one of the following:

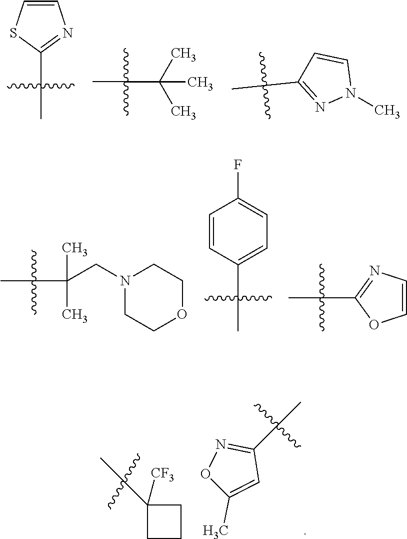

55. The bifunctional molecule according to any one of the preceding clauses, wherein the linker comprises 1 to 25 or 1 to 18 atoms in a single linear chain.

56. The bifunctional molecule according to any one of the preceding clauses, wherein linker comprises 1 to 10 or 1 to 8 rotatable bonds.

57. The bifunctional molecule according to any one of the preceding clauses, wherein the linker (L) is a covalent bond or the structure of the linker (L) is:

(Lₓ)_{q}
wherein each Lx represents a subunit of L that is independently selected from CR^{L1}R^{L2}, O, C═O, S, SO, SO₂, NR^{L3}, SONR^{L4}, SONR^{L5}C═O, CONR^{L6}, NR^{L7}CO, C(R^{L8})═C(R^{L9}), C≡C, aryl, substituted aryl, heteroaryl, substituted heteroaryl, carbocyclyl, substituted carbocyclyl, heterocyclyl and substituted heterocyclyl groups;
wherein R^{L1}, R^{L2}, R^{L3}, R^{L4}, R^{L5}, R^{L6}, R^{L7}, R^{L8} and R^{L9} are each independently selected from H, halo, C₁ to C₆ alkyl, C₁ to C₆, haloalkyl, —OH, —O(C₁ to C₆ alkyl), —NH₂, —NH(C₁ to C₆ alkyl), —NO₂, —CN, —CONH₂, —CONH(C₁ to C₆ alkyl), —CON(C₁ to C₆ alkyl)₂, —SO₂(C₁ to C₆ alkyl), —CO₂(C₁ to C₆ alkyl), and —CO(C₁ to C₆ alkyl); and
q is an integer between 1 and 30.

58. The bifunctional molecule according to any one of the preceding clauses, wherein the bifunctional molecule is not:

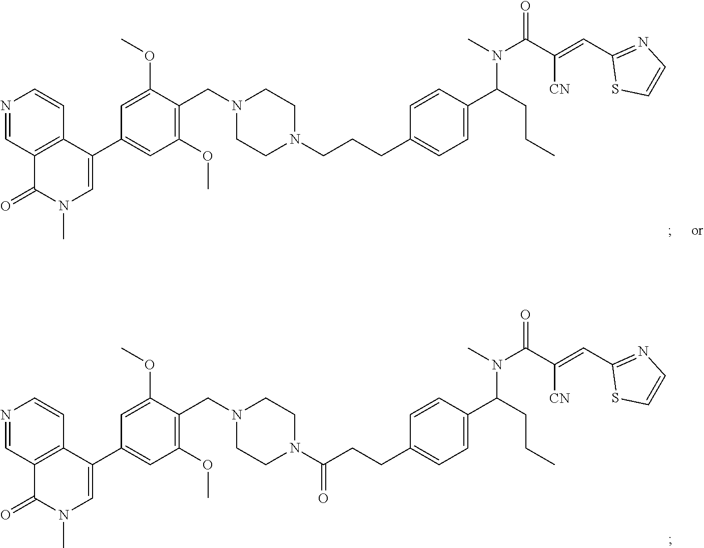 [SMILES: CCCC(c1ccc(CCCN2CCN(Cc3c(OC)cc(-c4cn(C)c(=O)c5cnccc45)cc3OC)CC2)cc1)N(C)C(=O)/C(C#N)=C/c1nccs1 ; CCCC(c1ccc(CCC(=O)N2CCN(Cc3c(OC)cc(-c4cn(C)c(=O)c5cnccc45)cc3OC)CC2)cc1)N(C)C(=O)/C(C#N)=C/c1nccs1]

or
wherein the BRD9 binder is not

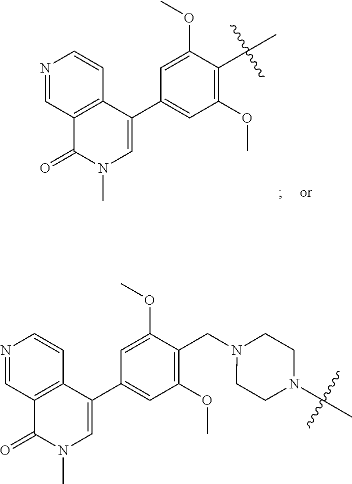 [SMILES: *c1c(OC)cc(-c2cn(C)c(=O)c3cnccc23)cc1OC ; *N1CCN(Cc2c(OC)cc(-c3cn(C)c(=O)c4cnccc34)cc2OC)CC1]

wherein the wavy line intersects the bond between the BRD9 binder and the linker.

59. A bifunctional molecule according to clause 1, wherein:
(i) Z is represented as formula (I), (Ia), (Ib), (Ic), (IIaa), (IIa) or (IIb) as defined above; and
(ii) TBL is represented by formula (1e), (1f) or (1f′) as defined above.

60. A bifunctional molecule according to clause 1, wherein:
(i) Z is represented as formula (Ia), (IIaa), or (IIa) as defined above; and
(ii) TBL is represented by formula (1e), (1f) or (1f′) as defined above.

61. A bifunctional molecule according to clause 1, wherein:
(i) Z is represented as formula (Ia), (IIaa), or (IIa) as defined above; and
(ii) TBL is represented by formula (1h), (1i) or (1j) as defined above.

62. A bifunctional molecule according to clause 1, wherein:
(i) Z is represented as formula (Ia), (IIaa) or (IIa) as defined above;
(ii) TBL is represented by formula 1a″ as defined above; and
(iii) L is represented by formula L1a or L1b.

63. A bifunctional molecule according to clause 1, wherein:
(i) Z is represented as formula (Ia), (IIaa) or (IIa) as defined above;
(ii) TBL is represented by any one of formulae 1e″, 1g″, 1g′″, 1ea″ to 1eh″, 1ea″, 1h″ to 1z″ and 2a″ to 2g″ as defined above; and
(iii) L is represented by formula L1a or L1b as defined above.

64. A bifunctional molecule according to clause 1, wherein:
(i) Z is represented as formula (Ib), or (IIb) as defined above; and
(ii) TBL is represented by formula (1e), (1f) or (1f′) as defined above.

65. A bifunctional molecule according to clause 1, wherein:
(i) Z is represented as formula (Ib), or (IIb) as defined above; and
(ii) TBL is represented by formula (1h), (1i) or (1j) as defined above.

66. A bifunctional molecule according to clause 1, wherein:
(i) Z is represented as formula (WI), (WII), (WIIa), (WIIb), (WIIc), (WIId), (WIIe), (WIIf), (WII), (WIIIa), (WIIIb), (WIV) or (WIVa) as defined above; and
(ii) TBL is represented by formula (1e), (1f) or (1f′) as defined above.

67. A bifunctional molecule according to clause 1, wherein:
(i) Z is represented as any one of formula (WZI), (WZII), (WZIIa) to (WZIIe), (WZIIIa) to (WZIIIh) or (WZIVa) to (WZIVj) as defined herein;
(ii) TBL is represented by formula 1a″ as defined herein; and
(iii) L is represented by formula L1c as defined herein.

68. A bifunctional molecule according to clause 1, wherein:
(i) Z is represented as any one of formula (WZI), (WZII), (WZIIa) to (WZIIe), (WZIIIa) to (WZIIIh) or (WZIVa) to (WZIVj) as defined herein;
(ii) TBL is represented by any one of formulae 1e″, 1g″, 1g′″, 1ea″ to 1eh″, 1ea″, 1h″ to 1z″ and 2a″ to 2g″ as defined herein; and
(iii) L is represented by formula L1c as defined herein.

69. A bifunctional molecule according to clause 1, wherein:
(i) Z is represented as formula (WI), (WII), (WIIa), (WIIb), (WIIc), (WIId), (WIIe), (WIIf), (WIII), (WIIIa), (WIIIb), (WIV) or (WIVa) as defined above;
wherein Z is not:

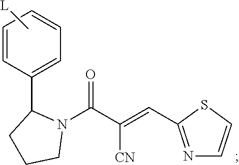

and
(ii) TBL is the target protein binding ligand that binds BRD9, wherein TBL is not:

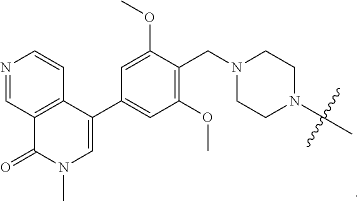 [SMILES: *N1CCN(Cc2c(OC)cc(-c3cn(C)c(=O)c4cnccc34)cc2OC)CC1]

70. The bifunctional molecule according to any one of the preceding clauses, wherein the bifunctional molecule has a structure as shown in Table 1.

71. A pharmaceutical composition comprising the bifunctional molecule according to any one of the preceding clauses, together with a pharmaceutically acceptable carrier, optionally wherein the bifunctional molecule is present in the composition as a pharmaceutically acceptable salt, solvate or derivative.

72. The bifunctional molecule according to any one of clauses 1 to 70 or the pharmaceutical composition of clause 71, for use in medicine.

73. The bifunctional molecule or pharmaceutical composition for use of clause 72, wherein the use comprises the treatment and/or prevention of any disease or condition which is associated with and/or is caused by an abnormal level of BRD9 activity.

74. The bifunctional molecule or pharmaceutical composition for use of clause 72 or 73, wherein the disease or condition is cancer.

75. A method of treating and/or preventing any disease or condition which is associated with and/or is caused by an abnormal level of BRD9 activity, the method comprising administering a therapeutically effective amount of a bifunctional molecule as defined in any one of clauses 1 to 70, or the pharmaceutical composition of clause 71 to a subject in need thereof.

76. The method of clause 75, wherein the disease or condition is cancer.

77. A method of selectively degrading and/or increasing proteolysis of BRD9 in a cell, the method comprising contacting and/or treating the cell with a bifunctional molecule as defined in any one of clauses 1 to 70 or a pharmaceutical composition as defined in clause 71.

78. Use of a bifunctional molecule as defined in any one of clauses 1 to 70 in a method of targeted BRD9 degradation.

79. A method of making a bifunctional molecule as defined in any one of clauses 1 to 70.

80. A method of screening bifunctional molecules according to any one of clauses 1 to 70, comprising:
a. providing a bifunctional molecule comprising:
   (i) a first ligand comprising a structure according to Z as defined in any one of clauses 1 and 21 to 54;
   (ii) a second ligand that binds to BRD9 as defined in any one of clauses 1 to 20; and
   (iii) a linker that covalently attaches the first and second ligands as defined in any one of clauses 1 and 55 to 57;
b. contacting a cell with the bifunctional molecule;
c. detecting degradation of BRD9 in the cell;
d. detecting degradation of BRD9 in the cell in the absence of the bifunctional molecule; and
e. comparing the level of degradation of BRD9 in the cell contacted with the bifunctional molecule to the level of degradation of BRD9 in the absence of the bifunctional molecule;
   wherein an increased level of degradation of BRD9 in the cell contacted with the bifunctional molecule indicates that the bifunctional molecule has facilitated and/or promoted the degradation of BRD9,
   optionally wherein detecting degradation of BRD9 comprises detecting changes in the levels of the target protein in the cell.

81. A compound library comprising a plurality of bifunctional molecules according to any one of clauses 1 to 70.

82. A kit of parts comprising:
(i) a first ligand comprising a structure according to Z as defined in any one of clauses 1 and 21 to 54;
(ii) a second ligand that binds to BRD9 as defined in any one of clauses 1 to 20; and separately
(iii) a linker that covalently attaches the first and second ligands as defined in any one of clauses 1 and 55 to 57.

### Definitions

In the present disclosure, reference is made to a number of terms, which are to be understood to have the meanings provided below, unless a context indicates to the contrary. The nomenclature used herein for defining compounds, in particular the compounds described herein, is intended to be in accordance with the rules of the International Union of Pure and Applied Chemistry (IUPAC) for chemical compounds, specifically the “IUPAC Compendium of Chemical Terminology (Gold Book)” (see A. D. Jenkins et al., Pure & Appl. Chem., 68, 2287-2311 (1996)). For the avoidance of doubt, if an IUPAC rule is contrary to a definition provided herein, the definition herein is to prevail.

As used herein, the term “alkyl” refers to a straight or branched chain hydrocarbyl group. The chain may be saturated or unsaturated, e.g. in some cases the chain may contain one or more double or triple bonds.

As used herein, “C₁-Cₙalkyl” may be selected from straight or branched chain hydrocarbyl groups containing from 1 to n carbon atoms. For example, “C₁-C₆alkyl” may be selected from straight or branched chain hydrocarbyl groups containing from 1 to 6 carbon atoms and C₁-C₃alkyl may be selected from straight or branched chain hydrocarbyl groups containing from 1 to 3 carbon atoms. Representative examples are methyl, ethyl, n-propyl, iso-propyl, n-butyl, sec-butyl, iso-butyl, tert-butyl, n-pentyl, isopentyl, neopentyl, n-hexyl, isohexyl, neohexyl, etc. When a C₁-C₆ alkyl group is substituted, any hydrogen atom(s), CH₃, CH₂ or CH group(s) may be replaced with the substituent(s), providing valencies are satisfied. Where the C₁-C₆ alkyl comprises a divalent hydrocarbon radical (containing from 1 to 6 carbon atoms), this moiety may sometimes be referred to herein as a C₁-C₆alkylene.

The term “cycloalkyl” defines all monovalent groups derived from cycloalkanes by removal of one hydrogen atom from a ring carbon atom. The term “cycloalkane” defines saturated monocyclic unbranched hydrocarbons, having the general formula CₙH₂ₙ, wherein n is an integer ≥3. As used herein, a “cycloalkyl” is a generally a ring containing 3 to 10 carbon atoms, in some cases 3 to 8, or in some cases 5 to 6 carbon atoms. The ring may be saturated or unsaturated, e.g. in some cases the ring may contain one or more double or triple bonds. As used herein, a Cₙ-C_{n′} cycloalkyl is a cycloalkyl containing n to n′ carbon atoms in the ring, where n and n′ are integers.

As used herein, “heterocycloalkyl” refers to a monocyclic or polycyclic ring having in one or more rings of the ring system at least one heteroatom selected from O, N and S (e.g. from one to five ring heteroatoms independently selected from the group consisting of O, N and S). The one or more rings may also contain one or more double bonds provided that the one or more rings are not fully aromaticized. The one or more rings of the heterocycloalkyl may comprise 3 to 10 atoms, in some cases 3 to 8 atoms. The one or more rings may be aliphatic. The one or more rings may be saturated or unsaturated, e.g. in some cases the one or more rings may contain one or more double or triple bonds. Any N heteroatom present in the heterocycloalkyl group may be C₁ to C₆ alkyl-substituted. In some cases, the heterocycloalkyl is a monocyclic or bicyclic ring, such as a monocyclic ring. A Cₙ-C_{n′}heterocycloalkyl is a heterocycloalkyl containing n to n′ carbon atoms in the ring, where n and n′ are integers. Representative examples of heterocycloalkyl groups include, but are not limited to, pyrrolidinyl, tetrahydrofuranyl, dioxolanyl, dithiolanyl, thiazolidinyl, isothiazolidinyl, oxazolidinyl, isoxazolidinyl, pyrazolidinyl, imidazolidinyl, piperidinyl, piperazinyl, N-alkylpiperazinyl, morpholinyl, dioxanyl, oxazolidinyl, tetrahydropyranyl, diazaspiroundecane, diazaspiroheptane, azaspiroheptane, diazaspirodecane, octahydropyrrolopyrrole, etc. As used herein, “substituted heterocycloalkyl” refers to a heterocycloalkyl group as defined herein which comprises one or more substituents on the heterocycloalkyl ring.

The term “heterocyclyl” refers to a monovalent radical derived from a heterocycle. A heterocycle is a cyclic compound (a compound comprising one or more rings of connected atoms) having as ring members atoms of at least two different elements (such as carbon and nitrogen).

As used herein, a “halo” group may be F, Cl, Br, or I, typically F.

The term “haloalkyl” refers to alkyl groups in which at least one hydrogen atom has been replaced with a halo atom, such as fluoro, chloro or bromo, often fluoro. By way of example, C₁-C₆haloalkyl refers to an alkyl group containing from 1 to 6 carbon atoms in which at least one hydrogen atom has been replaced with a halo atom. Trifluoromethyl and 1,1-difluoroethyl are examples of haloalkyls.

The term “alkenyl” is well known in the art and defines monovalent groups derived from alkenes by removal of a hydrogen atom from any carbon atom, wherein the term “alkene” is intended to define acyclic branched or unbranched hydrocarbons having the general formula CnH₂ₙ, wherein n is an integer ≥2. Examples of alkenyl groups include ethenyl, n-propylenyl, iso-propylenyl, n-butylenyl, sec-butylenyl, iso-butylenyl and tert-butylenyl. When an alkenyl group is substituted, any hydrogen atom(s) may be replaced with the substituent(s), providing valencies are satisfied. Where the alkenyl comprises a divalent hydrocarbon radical, this moiety may sometimes be referred to herein as an alkenylene.

The term “alkynyl” is well known in the art and defines monovalent groups derived from alkynes by removal of a hydrogen atom from any carbon atom, wherein the term “alkyne” is intended to define acyclic branched or unbranched hydrocarbons having the general formula CnH₂ₙ₋₂, wherein n is an integer ≥2. Examples of alkynyl groups include ethynyl, n-propylynyl, iso-propylynyl, n-butylynyl, sec-butylynyl, iso-butylynyl and tert-butylynyl. When an alkynyl group is substituted, any hydrogen atom(s) may be replaced with the substituent(s), providing valencies are satisfied. Where the alkynyl comprises a divalent hydrocarbon radical, this moiety may sometimes be referred to herein as an alkynylene.

“Benzyl” as used herein refers to a —CH₂Ph group. As used herein, a “substituted benzyl” refers to a benzyl group as defined herein which comprises one or more substituents on the CH₂ and/or the aromatic ring. When a benzyl group is substituted, any hydrogen atom(s) may be replaced with the substituent(s), providing valencies are satisfied.

As used herein, the term “aryl” refers to a mono- or polycyclic aromatic hydrocarbon system having 6 to 14 carbon atoms, in some cases having 6 to 10 carbon atoms. Representative examples of suitable “aryl” groups include, but are not limited to, phenyl, biphenyl, naphthyl, 1-naphthyl, 2-naphthyl and anthracenyl. As used herein, “substituted aryl” refers to an aryl group as defined herein which comprises one or more substituents on the aromatic ring. When an aryl group is substituted, any hydrogen atom(s) may be replaced with the substituent(s), providing valencies are satisfied.

As used herein, “heteroaryl” may be a single or fused ring system having one or more aromatic rings containing 1 or more, in some cases 1 to 3, in some cases 1 to 2, in some cases a single O, N and/or S heteroatom(s). The term “heteroaryl” may refer to a mono- or polycyclic heteroaromatic system having 5 to 10 ring atoms. A Cₙ-C_{n′}heteroaryl is a heteroaryl containing n to n′ carbon atoms in the ring, where n and n′ are integers. Representative examples of heteroaryl groups may include, but are not limited to, pyrrolyl, furanyl, thiophenyl, pyrazolyl, imidazolyl, oxazolyl, isoxazolyl, thiazolyl, pyridinyl, pyrimidinyl, pyridazinyl, pyrazinyl, indolyl, benzofuranyl, benzothiazolyl, benzimidazolyl, indazolyl, benzoxazolyl, benzisoxazolyl etc. As used herein, “substituted heteroaryl” refers to a heteroaryl group as defined herein which comprises one or more substituents on the heteroaromatic ring.

As used herein, a “carbocyclic ring” is a ring containing 3 to 10 carbon atoms, in some cases 3 to 8 carbon atoms, or in some cases 5 to 6 carbon atoms. The ring may be aliphatic. Thus, as used herein, references to “carbocyclyl” and “substituted carbocyclyl” groups may refer to aliphatic carbocyclyl groups and aliphatic substituted carbocyclyl groups. The ring may be saturated or unsaturated, e.g. in some cases the ring may contain one or more double or triple bonds. A Cₙ-C_{n′}carbocyclic ring is a carbocyclic ring containing n to n′ carbon atoms in the ring, where n and n′ are integers. Representative examples of carbocyclyl groups include cyclopropyl, cyclobutyl, cyclopentyl, cyclohexyl, cycloheptyl, cyclooctyl, cyclobutenyl, cyclopentenyl, cyclohexenyl, cycloheptenyl, cyclooctenyl, cyclooctynl etc. As used herein, “substituted carbocyclyl” refers to a carbocyclyl group as defined herein which comprises one or more substituents on the carbocyclic ring. When a carbocyclyl group is substituted, any hydrogen atom(s) may be replaced with the substituent(s), providing valencies are satisfied.

As used herein, a “heterocyclic ring” (or heterocyclyl) may comprise at least 1 heteroatom selected from O, N and S. The heterocyclic ring may be a monocyclic or polycyclic ring, each ring comprising 3 to 10 atoms, in some cases 3 to 8 atoms. The one or more rings may be aliphatic. Thus, as used herein, references to “heterocyclyl” and “substituted heterocyclyl” groups may refer to aliphatic heterocyclyl groups and aliphatic substituted heterocyclyl groups. The one or more rings may be saturated or unsaturated, e.g. in some cases the one or more rings may contain one or more double or triple bonds. A Cₙ-C_{n′}heterocyclic ring is a heterocyclic ring containing n to n′ carbon atoms in the ring, where n and n′ are integers. Any N heteroatom present in the heterocyclic group may be C₁ to C₆ alkyl-substituted. In some cases, the heterocyclyl is a monocyclic or bicyclic ring, such as a monocyclic ring. In other examples, the heterocyclyl may be a bicyclic ring, which may in some cases be a fused ring. Representative examples of heterocyclyl groups include, but are not limited to, pyrrolidinyl, tetrahydrofuranyl, dioxolanyl, dithiolanyl, thiazolidinyl, isothiazolidinyl, oxazolidinyl, isoxazolidinyl, pyrazolidinyl, imidazolidinyl, piperidinyl, piperazinyl, N-alkylpiperazinyl, morpholinyl, dioxanyl, oxazolidinyl, tetrahydropyranyl, diazaspiroundecane, diazaspiroheptane, azaspiroheptane, diazaspirodecane, octahydropyrrolopyrrole, pyrrolizidinyl, thiophenyl etc. As used herein, “substituted heterocyclyl” refers to a heterocyclyl group as defined herein which comprises one or more substituents on the heterocyclic ring.

As used herein, the term “optionally substituted” means that the moiety may comprise one or more substituents.

As used herein, a “substituent” may include, but is not limited to, hydroxy, thiol, carboxyl, cyano (CN), nitro (NO₂), halo, haloalkyl (e.g. a C₁ to C₆ haloalkyl or a C₁ to C₄ haloalkyl), an alkyl group (e.g. C₁ to C₁₀ or C₁ to C₆, which may itself be unsubstituted or substituted with, for example, one or more selected from the group consisting of aryl, halo and hydroxy), an alkenyl group (e.g. C₂ to C₆), an alkynyl group (e.g. C₂ to C₆), aryl (e.g. phenyl and substituted phenyl for example benzyl or benzoyl), morpholino, N—C₁₋₆alkylenylmorpholine, alkoxy group (e.g. C₁ to C₆ alkoxy or C₁ to C₄ alkoxy), haloalkoxy (e.g. C₁ to C₄ haloalkoxy), aryloxy (e.g. phenoxy and substituted phenoxy), hydroxyalkynyl (e.g. C₂ to C₆). thioether (e.g. C₁ to C₆ alkyl or aryl thioether), alkylthio (e.g. C₁ to C₆alkylthio), cyanoalkyl (e.g. C₁ to C₆), oxo, keto (e.g. C₁ to C₆ keto), ester (e.g. C₁ to C₆ alkyl or aryl ester, which may be present as an oxyester or carbonylester on the substituted moiety), thioester (e.g. C₁ to C₆ alkyl or aryl thioester), alkylene ester (such that attachment is on the alkylene group, rather than at the ester function which is optionally substituted with a C₁ to C₆ alkyl or aryl group), amine (including monoalkylamino, dialkylamino, a five- or six-membered cyclic alkylene amine optionally substituted with one or more halo, further including a C₁ to C₆ alkyl amine or a C₁ to C₆ dialkyl amine which alkyl groups may be substituted with one or two hydroxyl groups, and also including alkylphenylamino or alkylphenyl(alkyl)amino groups), amido (including —C(O)NH₂, —C(O)NH(alkyl) such as —C(O)NH(C₁₋₄alkyl), —C(O)N(alkyl)₂ such as —C(O)N(C₁₋₄alkyl)₂, —NHC(O)alkyl such as —NHC(O)C₁₋₄alkyl, —NHC(O)(phenyl), —N(alkyl)C(O)(alkyl) such as —N(C₁₋₄alkyl)C(O)(C₁₋₄alkyl), —N(alkyl)C(O)(phenyl) such as —N(C₁₋₄alkyl)C(O)(phenyl), N—C₁₋₆alkylenylamino, amido (e.g. which may be substituted with one or two C₁ to C₆ alkyl groups (including a carboxamide which is optionally substituted with one or two C₁ to C₆ alkyl groups), aminoalkyl (e.g. C₁ to C₄ aminoalkyl), alkanol (e.g. C₁ to C₆ alkyl, C₁ to C₄ alkyl or aryl alkanol), or carboxylic acid (e.g. C₁ to C₆ alkyl or aryl carboxylic acid), sulfoxide, sulfone, sulfinamide, sulfonamide, and urethane (such as —O—C(O)—NR₂ or —N(R)—C(O)—O—R, wherein each R in this context is independently selected from C₁ to C₆ alkyl or aryl), a heteroaryl (which may itself be substituted, for example with one or more groups selected from halogen, —OH, —NH₂, —NH—C₁₋₃alkyl and —C₁₋₅alkyl, C₁₋₅haloalkyl, C₁₋₅alkoxy, C₁₋₄haloalkoxy, 1d (defined above), C₃₋₅azacycloalkyl, C₂₋₅alkenyl, C₂₋₅alkynyl, C₃₋₅cycloalkyl, wherein the —C₁₋₅alkyl group can be optionally substituted with 5-6 membered heteroaryl or phenyl), a heterocyclyl, arylalkyl (such as an arylC₁₋₄alkyl), heteroarylalkyl (such as a heteroarylC₁₋₄alkyl), —OC₁₋₄alkylphenyl, —C(O)alkyl such as —C(O)(C₁₋₄alkyl), —C(O)alkylphenyl such as C(O)(C₁₋₄alkylphenyl), —C(O)haloalkyl such as —C(O)(C₁₋₄haloalkyl), —SO₂(alkyl) such as —SO₂(C₁₋₄alkyl), —SO₂(phenyl), —SO₂haloalkyl such as OSO₂(C₁₋₄ haloalkyl), —SO₂NH₂, —SO₂NH(alkyl) such as —SO₂NH(C₁₋₄alkyl), —SO₂NH(phenyl), —NHSO₂(alkyl) such as —NHSO₂(C₁₋₄alkyl), —NHSO₂(phenyl), —NHSO₂(haloalkyl) such as —NHSO₂(C₁₋₄haloalkyl), —S—C₁₋₃haloalkyl, —CH₂C(O)N(R^{C})₂, —C₃₋₄alkynyl(NR^{C})₂, deuteroC₂₋₄alkynyl, (C₁₋₃alkoxy)haloC₁₋₃alkyl-, C³⁻⁶cycloalkyl (wherein said C₃₋₆cycloalkyl is optionally substituted with halo or C₁₋₃alkyl), azido, sulfonyl, HC(O)—, —CO₂R^{C}, or —CO₂N(R^{C})₂, wherein R^{C} is hydrogen or C₁₋₃alkyl.

The term “analogue”, when used herein, refers to a compound or moiety having structural similarity to a specific compound or moiety. Despite the structural similarity, an analogue may display different chemical and/or biological properties. An analogue may have about 90% similarity with a specific compound or moiety, i.e. it may share about 90% of its structure with the specific compound or moiety. In some examples, an analogue may have about 92%, 94%, 96% or 98% similarity with a specific compound or moiety.

As used herein, where a group comprising carbon atoms is defined as “saturated”, only single bonds bind the carbon atoms to one another. Where a group comprising carbon atoms is defined as “unsaturated”, at least two of the carbon atoms are connected by a double or triple bond. For the avoidance of doubt, unsaturated compounds may comprise any number of double and/or triple bonds.

The term “spiro” is used to refer to moieties comprising two or more ring systems, wherein at least two of the ring systems are connected by just one atom (typically a quaternary carbon atom).

“Monocyclic” is used herein to refer to moieties comprising one ring of atoms. “Bicyclic” is used herein to refer to moieties that feature two joined rings of atoms. “Tricyclic” is used herein to refer to moieties that feature three joined rings of atoms. “Polycyclic” is used herein to refer to moieties that comprise two or more joined rings. Unless the context indicates otherwise, bicyclic and polycyclic systems may comprise a fused ring system (in which at least two rings share a common bond). In other examples, the two or more rings may be joined by a bond between atoms on each of the two or more rings. In other examples, the bicyclic system may comprise a spiro centre (as defined above).

The term “bridged” is used herein to refer to a cyclic moiety, or ring, comprising two bridgehead atoms (typically two carbon atoms of the cyclic compound or ring) that are connected by one or more atoms lying outside of the ring (such as one to three atoms lying outside of the ring). Bridged rings comprise two rings sharing three or more atoms. In some examples, the bridgehead atoms are separated within the ring by at least one carbon atom. In some examples, a ring may be bridged by between 1 and 3 bridging atoms which lie outside of the ring to form a bridging group (optionally wherein the bridging atoms are selected from C, N, O and S). As used herein, a “C₁₋₃ bridge” is a bridging group comprising between 1 and 3 carbon bridging atoms. The bridging group may comprise one to three atoms lying outside of the ring, of which one, two or three of those atoms are carbon. In some cases, the bridging group may additionally comprise non-carbon atoms (such as a heteroatom selected from N, O and S). By way of example, as used herein, a “C₁₋₃ bridge” may refer to a bridging group comprising between 1 and 3 atoms of which one, two or three are carbon and the remainder (if any) are selected from N, O and S. The bridging group may be a C₁ to C₃ alkylene (such as methylene, ethylene or propylene). The C₁ to C₃ alkylene bridging group may be optionally substituted with any suitable substituent as described herein. For example, C₁ to C₃ alkylene bridging group may be optionally substituted with one or two substituents each independently selected from the group consisting of halo, C₁ to C₃ alkyl, C₁ to C₃ haloalkyl and C₁ to C₃ alkoxy.

The term “fused” is used to refer to moieties comprising two or more ring systems, wherein at least two of the ring systems are connected by a [1,2] ring junction, i.e. a moiety comprising two or more ring systems wherein two, or more, of the rings present share a bond in each respective ring structure.

The term “aliphatic” refers to acyclic or cyclic, saturated or unsaturated moieties, excluding aromatic moieties, where “aromatic” defines a cyclically conjugated molecular entity with a stability (due to delocalisation) significantly greater than that of a hypothetical localised structure. The Hückel rule is often used in the art to assess aromatic character; monocyclic planar (or almost planar) systems of trigonally (or sometimes diagonally) hybridised atoms that contain (4n+2) π-electrons (where n is a non-negative integer) will exhibit aromatic character. The rule is generally limited to n=0 to 5.

The term “hydrocarbyl” refers to a monovalent radical derived from a hydrocarbon by the removal of a hydrogen atom from the hydrocarbon. A hydrocarbon is any molecule comprising only the elements carbon and hydrogen. Hydrocarbons may be aliphatic, aromatic, unsaturated or saturated.

As used herein, an alkoxy refers to an alkyl group, as defined above, appended to the parent molecular moiety through an oxy group, —O—. As used herein, a C₁-C₆alkoxy refers to a C₁-C₆ alkyl group (as defined above), appended to the parent molecular moiety through a oxy group, —O—, and a C₁-C₄alkoxy refers to a C₁-C₄alkyl group (as defined above), appended to the parent molecular moiety through a oxy group, —O—. Representative examples of alkoxy include, but are not limited to, methoxy, ethoxy, propoxy, 2-propoxy, butoxy, tert-butoxy, pentyloxy, hexyloxy etc.

The term “alkoxyalkyl” is used herein to refer to a moiety derived from an alkyl moiety in which a hydrogen atom at any position of the alkyl is substituted with an alkoxy moiety. Examples of alkoxyalkyl groups include methoxyethyl, methoxypropyl, ethoxymethyl and the like.

The term “alkylamino” is used herein to refer to a moiety derived from an amino (NH₂) moiety in which one or both hydrogen atom(s) of the amino is/are substituted with one or two alkyl moieties. Examples of alkylamino groups include dimethylamino, diethylamino and the like.

The term “alkylaminoalkyl” is used herein to refer to a moiety derived from an alkyl moiety in which a hydrogen atom at any position of the alkyl is substituted with an alkylamino moiety. Examples of alkylaminoalkyl groups include dimethylaminomethyl, dimethylaminoethyl and the like.

The term “alkoxyalkylene” is used herein to refer to a moiety derived from an alkylene moiety in which a hydrogen atom at any position of the alkylene is substituted with an alkoxy moiety. Examples of alkoxyalkylene groups include methoxyethylene, methoxymethylene and the like.

The term “haloalkylene” is used herein to refer to a moiety derived from an alkylene moiety in which one or more hydrogen atom(s) at any position(s) of the alkylene is/are substituted with one or more halo moieties. Examples of haloalkylene groups include fluoroethylene, difluoromethoxymethylene, dichloroethylene and the like.

The term “hydroxyalkylene” is used herein to refer to a moiety derived from an alkylene moiety in which a hydrogen atom at any position of the alkylene is substituted a hydroxy moiety. Examples of hydroxyalkylene groups include hydroxyethylene, hydroxymethylene and the like

In some examples, and unless the context indicates otherwise, a “substituent” may include, but is not limited to, halo, C₁ to C₆ alkyl, NH₂, NH(C₁ to C₆ alkyl), N(C₁ to C₆ alkyl)₂, OH, O(C₁ to C₆ alkyl), NO₂, CN, C₁-C₆ haloalkyl, CONH₂, CONH(C₁ to C₆ alkyl), CON(C₁ to C₆ alkyl)₂, C(O)OC₁ to C₆ alkyl, CO(C₁ to C₆ alkyl), S(C₁ to C₆ alkyl), S(O)(OC₁ to C₆ alkyl) and SO(C₁ to C₆ alkyl).

As used herein, an electron withdrawing group may refer to any group which draws electron density away from neighbouring atoms and towards itself. Typically, the electron withdrawing group draws electron density away from neighbouring atoms and towards itself more strongly than a hydrogen substituent. Representative examples of suitable electron withdrawing groups include, but are not limited to, —CN, halo, —NO₂, —CONH₂, —CONH(C₁ to C₆ alkyl), —CON(C₁ to C₆ alkyl)₂, —SO₂(C₁ to C₆ alkyl), —CO₂(C₁ to C₆ alkyl), —CO(C₁ to C₆ alky) and C₁ to C₆ haloalkyl.

It should be understood that throughout this specification, the terms “comprise”, “comprising” and/or “comprises” is/are used to denote that aspects, embodiments and examples of this disclosure “comprise” a particular feature or features. It should be understood that this/these terms may also encompass aspects, embodiments and/or examples which “consist essentially of” or “consist of” the relevant feature or features.

### DETAILED DESCRIPTION

The present invention will now be described in detail with reference to the following non-limiting examples.

List of Abbreviations:

μL = Microliter
μM = Micromolar
NMR = Nuclear Magnetic Resonance
ACN = acetonitrile
AcOH or HOAc = acetic acid
BINAP = (2,2′-bis(diphenylphosphino)-1,1′-binaphthyl)
Boc = tert-butoxycarbonyl
bs = broad singlet
° C. = degrees Celsius
d = doublet
δ = chemical shift
DCM = Dichloromethane
dba = dibenzylideneacetone
DIPEA = N,N-Diisopropylethylamine, or Hünig's base
DMF = N,N-dimethylformamide
DMSO = Dimethylsulfoxide
dppf = 1,1′-Ferrocenediyl-bis(diphenylphosphine)
EtOAc = Ethyl acetate
g or G = gram
h or H = Hour(s)
HATU = 1-[Bis(dimethylamino)methylene]-1H-1,2,3-
triazolo[4,5-b]pyridinium 3-oxide hexafluorophosphate
HPLC = high performance liquid chromatography
Hz = Hertz
J = coupling constant (given in Hz unless otherwise indicated)
LCMS = liquid chromatography mass spectrometry
m = multiplet
M = Molar
M + H⁺ = parent mass spectrum peak plus H⁺
mg = Milligram
min = minutes
mL = Milliliter
mM = Millimolar
mmol = Millimole
MS = mass spectrum
MSCl = methanesulfonyl chloride
MTBE = methyl tert-butyl ether
nM = nanomolar
NMP = N-Methyl-2-pyrrolidone
pTsOH = p-toluenesulfonic acid
q = quartet
RT or r.t. = room temperature
STAB = sodium triacetoxyborohydride
t = triplet
TBAF = tetra-n-butylammonium fluoride
TFA = trifluoroacetic acid
THF = tetrahydrofuran
TLC = thin layer chromatography
XPhos = 2-Dicyclohexylphospino-2′,4′-6′-triisopropylbiphenyl

### Chemistry—Materials and Methods

All chemicals, unless otherwise stated were commercially available and used without further purification. Solvents were anhydrous and reactions preformed under positive pressure of nitrogen or argon.

Flash column chromatography (FCC) was performed using a Teledyne Isco Combiflash Rf or Rf200i. Prepacked columns RediSep Rf Normal Phase Disposable Columns were used. NMR data was acquired in Bruker Avance Neo nano bay 400 MHz NMR Spectrometer. Chemical Shifts are reported in ppm relative to dimethyl Sulfoxide (δ 2.50), methanol (δ 3.31), chloroform (δ7.26) or other solvent as indicated in NMR spectral data. A small amount (1-5 mg) of sample is dissolved in an appropriate deuterated solvent (0.8 ml).

Preparative HPLC was performed on a Gilson Preparative HPLC System with a Waters X-Bridge C18 column (100 mm×19 mm; 5 μm particle size) and a gradient of 5% to 95% acetonitrile in water over 10 min, flow 25 mL/min, with 0.1% formic acid in the aqueous phase.

Liquid Chromatography Mass Spectra (LC-MS) were recorded using positive ion electron spray ionisation (ESI⁺) on an Agilent InfinityLab Single Quadrupole LC/MSD with a Waters XBridge® C18 3.5 μm column (2.1 mm×50 mm) using H₂O+MeCN (5-95%)+0.1% HCO₂H or H₂O+MeCN (20-95%)+0.1% HCO₂H as eluent, using a linear gradient over 3 minutes. Alternatively, a Shimadzu LC; Prominence-I series instrument was used, with the following set up:

HPLC Method A

| Column: X-Bridge C8 (150 × 4.6 mm, 5.0 μm) | |
|---|---|
| Detection: UV @ 210-400 nm(Max Plot) | |
| Sample Diluent: Acetonitrile and Water | |
| Mobile Phase A: 10 mM Ammonium Acetate in water | |
| Mobile Phase B: Acetonitrile | |
| Flow rate: 1.5 mL/Min | |
| Runtime: 12.0 Min | |
| Elution: Gradient elution | |
| Time in Min | % of Mobile Phase B |
| 0.01 | 10 |
| 8.0 | 100 |
| 10.0 | 100 |
| 10.01 | 10 |
| 12.0 | 10 |

HPLC Method B

| Column: X-select CSH C18 (150 × 4.6 mm, 5.0 μm) | |
|---|---|
| Column Temperature: Ambient | |
| Detection: UV @ 210-400 nm(Max Plot) | |
| Sample Diluent: Acetonitrile and Water | |
| Mobile Phase A: 0.1% Formic acid in water | |
| Mobile Phase B: Acetonitrile | |
| Runtime: 10.0 Min | |
| Flowrate: 2.0 mL/Min | |
| Elution: Gradient elution | |
| Time in Min | % of Mobile Phase B |
| 0 | 5 |
| 8 | 100 |
| 8.01 | 5 |
| 10 | 5 |

### Part A—Synthetic Methods

Overviews of various exemplary synthetic methods and general procedures that may be used to provide the compounds of the present disclosure are shown below.

### Reductive Amination—General Procedure I

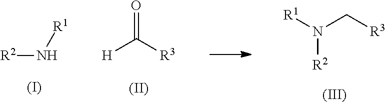

A solution of amine (I) (1 equiv.) and aldehyde (II) (1 equiv.) in DCM (0.05 M) was treated with Et₃N (1.5 equiv.). The reaction mixture was stirred for 1 h, then was treated with NaBH(OAc)₃ (2.0 equiv.) or MP-CNBH₃ (5.0 equiv.) (or alternative reducing agent, as noted in the procedure). The reaction mixture was stirred at room temperature until the reaction was complete by LCMS. The reaction was quenched by addition of H₂O and extracted with DCM. The combined organic extracts were washed with water, brine, dried over MgSO₄ and concentrated in vacuo. Purification by silica gel column chromatography yielded the desired product.

### Reductive Amination—General Procedure 14

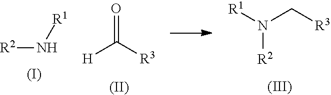

A solution of amine (I) (1 equiv.) and aldehyde (II) (1 equiv.) in solvent (noted below. 0.05 M) was treated with either:
a) acetic acid (catalytical amount), solvent=MeOH (procedure 14)
b) sodium acetate (2 equiv.), solvent=DCM:MeOH (1:1) (procedure 14a)
c) sodium acetate (2 equiv.) and acetic acid (catalytic amount), solvent=DCM:MeOH) (procedure 14b=30)

The reaction mixture was stirred for 1 h, then was treated with NaBH(OAc)₃ (2.0 equiv.) or MP-CNBH₃ (w/w) (or alternative reducing agent, as noted in the procedure). The reaction mixture was heated 70° C. for overnight. The completion of reaction was checked by LCMS. The reaction mixture was concentrated under vacuum and quenched by addition of H₂O and extracted with DCM. The combined organic extracts were washed with water, brine, dried over MgSO₄ and concentrated in vacuo. Purification by silica gel column chromatography yielded the desired product.

### Boc Deprotection—General Procedure 2

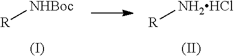

A solution of Boc protected amine (I) (1.0 equiv.) in DCM (procedure 2) or 1,4-dioxane (procedure 2a) (0.05 M) was treated with HCl (4 M In dioxane, 50 equiv.) and the mixture was stirred for 2 h. The volatiles were evaporated in vacuo to yield the corresponding amine hydrochloride (II).

### Acetylation

### Boc Deprotection—General Procedure 13

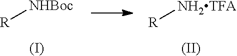

A solution of Boc protected amine (I) (1.0 equiv.) in DCM (5 mL) was added with TFA (2 equiv.) at 0° C. and stirred at room temperature for 3 h. The reaction was monitored by TLC; after completion, the reaction mixture was concentrated under reduced pressure. The crude material was purified by medium pressure liquid chromatography to afford the corresponding amine trifluoroacetic acid (II).

### Amine Acylation—General Procedure 3

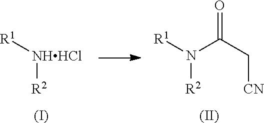

To a suspension of amine (I) (1.0 equiv.) in either
a) 1,4-dioxane (0.05 M) was treated with Et₃N (3 equiv.) (procedure 3) or DIPEA (3 equiv.) (procedure 3a); or
b) MeCN (0.05 M) was treated with Et₃N (3 equiv.) (procedure 3b);
was added 3-(3,5-dimethyl-1H-pyrazol-1-yl)-3-oxopropanenitrile (1.1 equiv.) and the mixture was heated to 80° C. (procedure 3/3a) or 55° C. (procedure 3b) for 16 h. The volatiles were concentrated in vacuo and purified by flash chromatography to yield the corresponding cyanoacetamide (II).

### Cyano-Knoevenagel Condensation—General Procedure 4

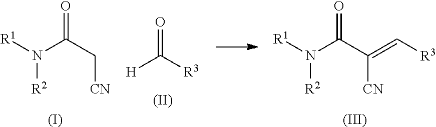

A solution of cyanoacetamide (I) (1.0 equiv.) in THF (procedure 4) (0.1 M) or EtOH (procedure 4a) was treated with aldehyde (II) (2.5 equiv.) and piperidine (0.5 equiv.) and the mixture was stirred at RT or heated to reflux for 72 h until the reaction was complete. The volatiles were concentrated in vacuo and purified by silica gel column chromatography to yield the corresponding cyanoacrylamide (III).

### Cyano-Knoevenagel Condensation—General Procedure 17a

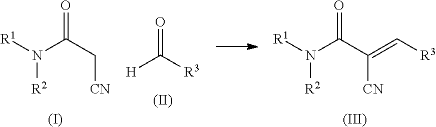

A solution of cyanoacetamide (I) (1.0 equiv.) in DCM/DMF (0.1 M) was treated with pyrrolidine (5 eq) and TMS-Cl (4 eq) followed by addition of aldehyde (II) (5 equiv.), and the mixture was stirred at or heated to 55° C. for 16 h until the reaction was complete RT. The volatiles were concentrated in vacuo (at low temperature where appropriate) and purified by silica gel column chromatography to yield the corresponding cyanoacrylamide (III).

### Cyano-Knoevenagel Condensation—General Procedure 17b

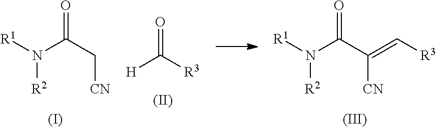

A solution of cyanoacetamide (I) (1.0 equiv.) and aldehyde (II) (4 equiv.) in ethanol:water (2:1, 0.064 M) was treated with beta-alanine (16.0 eq) and the mixture was for 16 h at RT. The volatiles were concentrated in vacuo and purified by silica gel column chromatography to yield the corresponding cyanoacrylamide (III).

### Cyano-Knoevenagel Condensation—General Procedure 17c

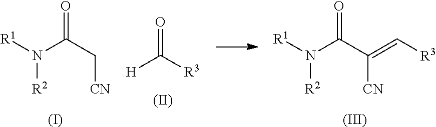

A solution of cyanoacetamide (I) (1.0 equiv.) and aldehyde (II) (4 equiv.) in DCM/DMA (1:1) was added pyrrolidine (0.6 ml) followed by acetic acid (1 eq) and the mixture was for 16 h at RT. The volatiles were concentrated in vacuo and purified by silica gel column chromatography to yield the corresponding cyanoacrylamide (III).

### Cyano-Knoevenagel Condensation—General Procedure 17d

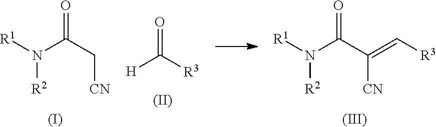

A solution of cyanoacetamide (I) (1.0 equiv.) and aldehyde (II) (4 equiv.) in DMA (0.6 mL) was treated with acetic acid (1 eq) and the mixture was for 16 h at RT. The volatiles were concentrated in vacuo and purified by silica gel column chromatography to yield the corresponding cyanoacrylamide (III).

### Ester Hydrolysis—General Procedure 5

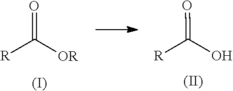

A solution of ester (I) (1.0 equiv.) in THF (0.2 M) was treated with lithium hydroxide monohydrate (3.0 equiv.) dissolved in water and the mixture was stirred for 4 h. The 25 mixture was adjusted to pH ˜3 by addition of 5% KHSO₄ and extracted with EtOAc. The combined organic layers were washed with water and brine, dried over MgSO₄ and concentrated in vacuo to yield the corresponding carboxylic acid (II).

### Ester Hydrolysis—General Procedure 7a

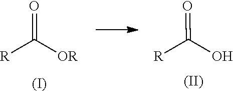

A solution of ester (I) (1.0 equiv.) in DCM (0.2 M) was treated with TFA (10.0 equiv.) and the mixture was stirred for 4 h. The mixture was concentrated under reduced pressure and purified by medium pressure liquid chromatography to yield the corresponding carboxylic acid (II).

### Amide Coupling—General Procedure 6

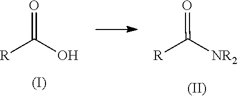

To a stirred solution of carboxylic acid (I) (1.0 equiv.) in DMF was added DIPEA (2.5 equiv.) and HATU (1.5 equiv.). The reaction mixture was stirred for 5 min, then relevant amine (1.5 equiv.) was added and the reaction mixture was stirred for 16 h at RT. The reaction was quenched with Ice cold water and extracted with EtOAc. The combined organic layers were concentrated in vacuo to afford the crude product Mere stated, the crude product was purified by silica gel column chromatography/reverse phase preparative HPLC to give the desired amide (II).

### COCF₃ Deprotection—General Procedure 20

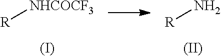

A solution of COCF₃ protected amine (I) (1.0 equiv.) in MeOH:Water (1:1) was treated with K₂CO₃ (5 equiv.) and the mixture was stirred for 16 h at RT. The volatiles were evaporated in vacuo to yield the corresponding amine (II).

### Buchwald Coupling—General Procedure 21

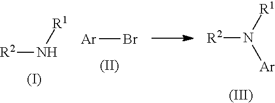

A solution of amine (I) (1.1 equiv.) and aryl-bromide (II) (1 equiv.) in 1,4-dioxane was treated with cesium carbonate (3 equiv.). The reaction mixture was degassed with N2 gas for 10 mins, then XPhos Pd G₄ (0.1 equiv.) (procedure 21) or Pd2(dba)3 (0.1 equiv.) (procedure 21a) was added and the reaction stirred at 100° C. overnight. The reaction mixture was filtered through celite and the filtrate was washed with ethyl acetate. The combined organic layers were concentrated in vacuo. Purification by silica gel column chromatography yielded the desired product.

### Trifluoroacetate Protection—General Procedure 22

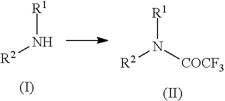

A solution of amine (I) (1 equiv.) in DCM at 0° C. was treated with triethylamine (3 equiv.) followed by trifluoroacetic acid anhydride (1.5 equiv) dropwise for 15 mins. The reaction mixture was stirred at RT overnight before being concentrated in vacuo and purified by silica gel column chromatography to yield the desired product.

### Phenol Formation—General Procedure 24

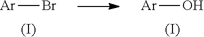

A solution of aryl-bromide (I) (1 equiv.), tBuXPhos-Pd-G₃ (0.05 equiv.) and KOH (1M, 6 equiv.) in 1,4-dioxane was heated to 110° C. for 2 h. The reaction was quenched with ice-water and extracted with ethyl acetate. The organic phases were dried over anhydrous Na₂SO₄ and concentrated in vacuo to afford the crude product which was purified by silica gel column chromatography.

### Mesylate Formation—General Procedure 25

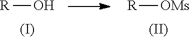

A solution of alcohol (I) (1 equiv.) and triethylamine (4 equiv.) in DCM was treated with mesyl chloride (3 equiv.). The reaction mixture was stirred at RT for 2 h before being quenched with water and extracted with DCM. The combined organic phases were dried over anhydrous sodium sulphate, filtered and concentrated in vacuo. The crude product was purified by silica gel column chromatograph to yield the desired product.

### OH/NH Alkylation—General Procedure 26

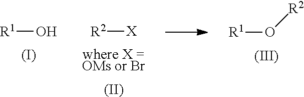

A solution of phenol (I) (1.1 equiv.) and mesylate or bromo (II) (1.7 equiv.) in acetonitrile was treated cesium carbonate (3 equiv.). The reaction mixture was stirred at RT overnight before being concentrated in vacuo and partitioned between ice-water and DCM. The aqueous phase was extracted with DCM and the combined organic phases were dried over anhydrous sodium sulphate, filtered and concentrated in vacuo. The crude product was purified by silica gel column chromatography to yield the desired product.

### Cbz Deprotection—General Procedure 27

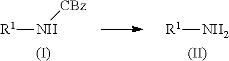

To a solution of Cbz-amine (I) (1 equiv.) in MeOH was added Pd/C under nitrogen atmosphere. The atmosphere was replace with hydrogen gas (1 atm) and the reaction mixture was stirred at RT overnight. The reaction mixture was filtered through celite and the celite bed was washed with MeOH. The filtrate was concentrated in vacuo to afford crude product which was used without further purification

### Olefin Installation—General Procedure 28

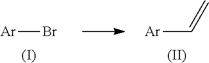

To a degassed solution of arylbromide (I) (1.0 equiv.), potassium trifluoro(vinyl)borate (1 equiv.) and Cs₂CO₃ (2 equiv.) in 1,4-dioxane:water (4:1) was added Pd(dppf)Cl₂·CH₂Cl₂ (0.1 equiv.) at room temperature. The reaction mixture was degassed for 10 mins before being heated at 90° C. for 16 h. The reaction mixture was filtered through celite and the celite was washed with EtOAc. The combined washings were concentrated in vacuo and the resulting residue was purified by silica gel column chromatography. The appropriate fractions were concentrated in vacuo to give the required product (II).

### Aldehyde Formation—General Procedure 29

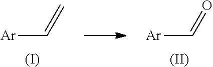

To a solution of olefin (I) (1.0 equiv.), sodium periodate (2 equiv.) and N-methyl morpholine (1 equiv.) in 1,4-dioxane:water (2:1) was added osmium tetroxide (4 wt % aqueous solution, 1 equiv.) dropwise at room temperature. The reaction mixture was stirred for 2 h at room temperature before being quenched with cold water and extracted with ethyl acetate. The combined organic layers were washed with brine and dried over anhydrous sodium sulphate, filtered and concentrated in vacuo. The resulting residue was purified by silica gel column chromatography. The appropriate fractions were concentrated in vacuo to give the desired aldehyde (II)

### Wittig Reaction—General Procedure 30

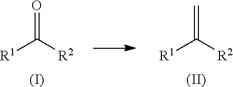

To a solution of methyltriphenylphosphonium bromide (2 equiv.) in THF was added potassium tert-butoxide (2 equiv.) portion-wise at 0° C. The reaction mixture was stirred for 30 min, then keton (I) (1 equiv.) in THF was added dropwise. The reaction mixture was stirred 1 h at room temperature before being quenched with ice-water and extracted with ethyl acetate. The organic phase was concentrated in vacuo and the resulting residue was purified by silica gel column chromatography. The appropriate fractions were concentrated in vacuo to afford desired olefin (1l).

### Heck Reaction—General Procedure 31

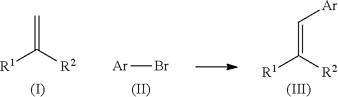

To a solution of aryl bromide (II) (1 equiv.) in DMF was added olefin (I) (1 equiv.) and K₂CO₃ (3 equiv.) at room temperature. The reaction mixture was purged with N₂ gas for 10 min then tri(o-tolyl)phosphine (0.1 equiv.) and PdOAc₂ (0.1 equiv.) were added. The reaction mixture was stirred at 100° C. overnight before being filtered through celite. The celite was washed with methanol and the washings were concentrated in vacuo. The resulting residue was purified by silica gel column chromatography. The appropriate fractions were concentrated in vacuo to afford product (III).

### Synthetic Pathways

### PREPARATIVE EXAMPLES

### Intermediate TBL-1: 2,6-dimethoxy-4-(5-methyl-4-oxo-4,5-dihydrothieno[3,2-c]pyridin-7-yl)benzaldehyde

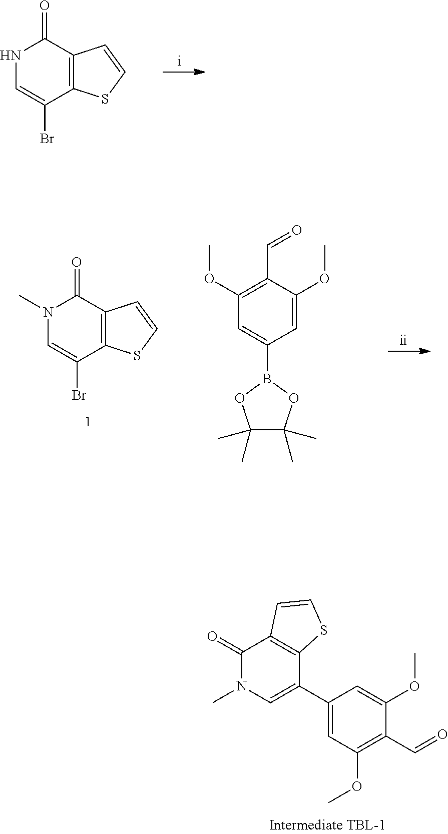 [SMILES: O=c1[nH]cc(Br)c2sccc12 ; Cn1cc(Br)c2sccc2c1=O ; COc1cc(B2OC(C)(C)C(C)(C)O2)cc(OC)c1C=O ; COc1cc(-c2cn(C)c(=O)c3ccsc23)cc(OC)c1C=O]

1: To a stirred solution of 7-bromothieno[3,2-c]pyridin-4(5H)-one (5 g, 21.7 mmol) in DMF (50 mL) was added K₂CO₃ (6.01 g, 43.5 mmol) at 0° C. The reaction mixture was stirred for 1 h at RT, then MeI (1.49 ml, 23.9 mmol) was added and the reaction was stirred for 16 h. The reaction mixture was quenched with ice-cold water and extracted with EtOAc. The combined organic layers were dried over anhydrous sodium sulphate and concentrated in vacuo. The crude product was purified silica gel column chromatography (gradient=10% MeOH in DCM). The appropriate fractions were concentrated in vacuo to afford 7-bromo-5-methylthieno[3,2-c]pyridin-4(5H)-one (4.5 g, 18.25 mmol, 84% yield). LCMS m/z [M+H]⁺=246.2

Intermediate TBL-1: A stirred solution of 7-bromo-5-methylthieno[3,2-c]pyridin-4(5H)-one (4.9 g, 20.1 mmol), 2,6-dimethoxy-4-(4,4,5,5-tetramethyl-1,3,2-dioxaborolan-2-yl)benzaldehyde (8.21 g, 28.1 mmol) and tripotassium phosphate (12.8 g, 60.2 mmol) in THF (120 mL) and water (20 mL) at RT was degassed with N₂ for 10 min. XPhos Pd G2 (0.79 g, 1.0 mmol]) was added to the reaction mixture which was heated at 80° C. for 5 h. The reaction mixture was then filtered through celite and washed with excess EtOAc. The filtrate was concentrated in vacuo and the crude compound was triturated with MTBE before being dried in vacuo to afford 2,6-dimethoxy-4-(5-methyl-4-oxo-4,5-dihydrothieno[3,2-c]pyridin-7-yl)benzaldehyde (6.0 g, 16.0 mmol, 88% yield). LCMS m/z [M+H]⁺=330.1

### Intermediate W-1 and W-2:

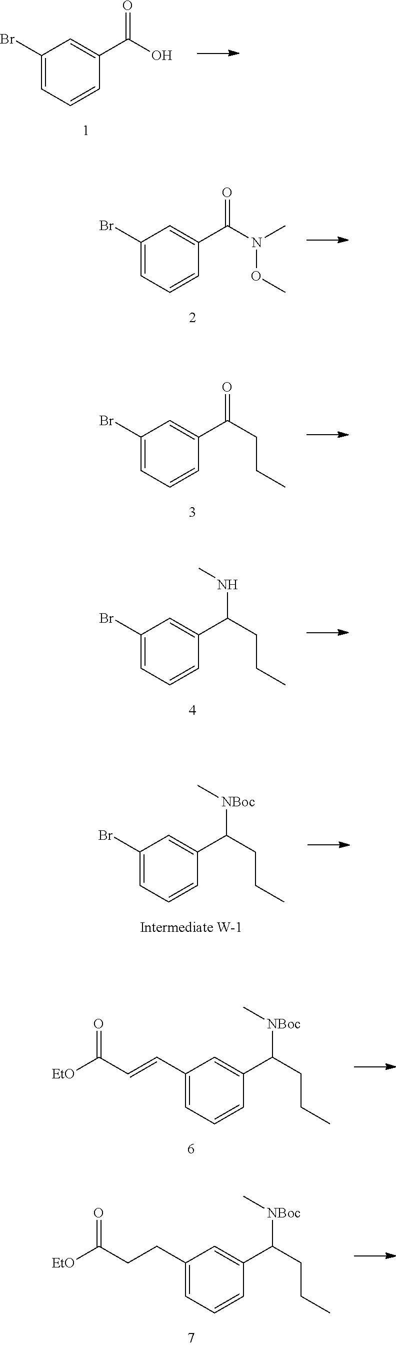

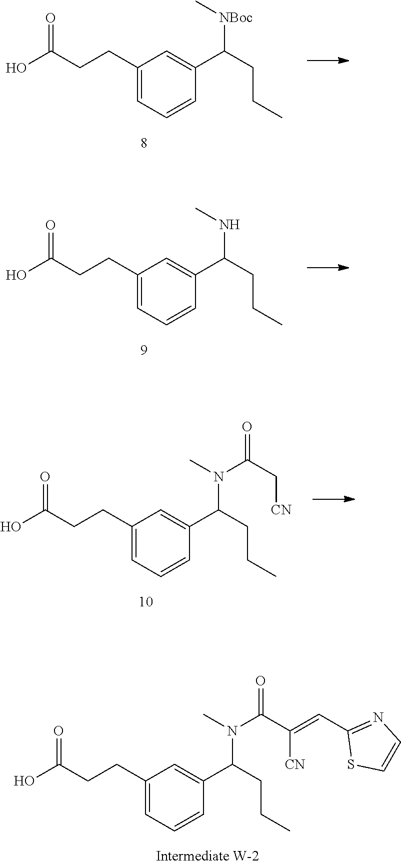 [SMILES: CCCC(c1cccc(CCC(=O)O)c1)N(C)C(=O)OC(C)(C)C ; CCCC(NC)c1cccc(CCC(=O)O)c1 ; CCCC(c1cccc(CCC(=O)O)c1)N(C)C(=O)CC#N ; CCCC(c1cccc(CCC(=O)O)c1)N(C)C(=O)/C(C#N)=C/c1nccs1]

2: To a stirring solution of 3-bromobenzoic acid (12.0 g, 59.69 mmol) in DCM (150 mL) were added EDC·HCl (17.10 g, 89.54 mmol) and DIPEA (20.30 mL, 119.39 mmol) at room temperature. After stirring for 10 min, methoxymethanamine hydrochloride (6.94 g, 71.63 mmol) was added. The reaction mixture was stirred at room temperature for 2 h. The reaction was monitored by TLC; after completion of the reaction, the reaction mixture was diluted with water and washed with DCM. The combined organic layers were dried over anhydrous Na₂SO₄, filtered and concentrated. The crude material was purified by column chromatography (gradient=20-25% EtOAc in heptane) to afford 3-bromo-N-methoxy-N-methylbenzamide (12.0 g, 82%) as pale yellow liquid; 1H NMR (400 MHz, CDCl₃) δ=7.83 (s, 1H), 7.67-7.56 (m, 2H), 7.34-7.22 (m, 1H), 3.55 (s, 3H), 3.36 (s, 3H). LC-MS: m/z=244.0 [M+H]⁺

3: To a stirring solution of 3-bromo-N-methoxy-N-methylbenzamide (18.0 g, 73.74 mmol) in THF (100 mL) was added propyl magnesium bromide (2.0 M in THF) (110.6 mL, 221.22 mmol) at −5° C. under inert atmosphere. The reaction mixture was stirred at room temperature for 5 h. The reaction was monitored by TLC; after completion of the reaction, the reaction mixture was quenched with ammonium chloride solution and extracted with EtOAc. The combined organic layers were dried over anhydrous Na₂SO₄, filtered and concentrated under reduced pressure. The crude material was purified by column chromatography (gradient=10-15% EtOAc in hexane) to afford 1-(3-bromophenyl)butan-1-one (10 g, 59%) as pale brown solid. LC-MS: m/z=227.0 [M+H]⁺

4: To a stirring solution of 1-(3-bromophenyl)butan-1-one (14.0 g, 61.64 mmol) in methanol (140 mL) was added were added MeNH₂ (2.0 M In THF) (92 mL, 185.02 mmol) and Ti(OiPr)₄ (18.76 g, 66.07 mmol) at 0° C. The reaction mixture was stirred at room temperature for 16 h. Then, NaBH₄ (2.81 g, 73.97 mmol) was added and stirring of the reaction mixture was continued at room temperature for 5 h. The reaction was monitored by TLC; after completion of the reaction, the reaction mixture was quenched with ice water and extracted with EtOAc. The combined organic layers were dried over anhydrous Na₂SO₄, filtered and concentrated under reduced pressure. The crude was purified by column chromatography by eluting with 15-20% EtOAc in heptane to afford 1-(3-bromophenyl)-N-methylbutan-1-amine (12 g, 80%) as yellow liquid. LC-MS: m/z=242.1 [M+H]⁺

Intermediate W-1: To a stirring solution of 1-(3-bromophenyl)-N-methylbutan-1-amine (10 g, 41.29 mmol) in THF (100 mL) were added Et₃N (11.5 mL, 82.59 mmol), DMAP (1.007 g, 8.25 mmol) and Boc₂O (13.5 g, 61.94 mmol) at room temperature. The reaction mixture was heated at 80° C. and stirred for 12 h. The reaction was monitored by TLC; after completion of the reaction, the reaction mixture was diluted with EtOAc and washed with water. The organic layers were dried over anhydrous Na₂SO₄, filtered and concentrated under reduced pressure. The crude was purified by column chromatography (gradient=20% EtOAc in heptane) to afford tert-butyl (1-(3-bromophenyl)butyl)(methyl)carbamate (Intermediate W-1) (8.0 g, 56%) as yellow liquid. LC-MS: m/z=242.2 [M-Boc+H]⁺

6: A mixture of tert-butyl (1-(3-bromophenyl)butyl)(methyl)carbamate (Intermediate W-1) (1.0 g, 2.92 mmol) and Et₃N (1.0 mL, 8.76 mmol) in DMF (10 mL) was degassed with argon for 15 min. Then, ethyl acrylate (880 mg, 8.76 mmol) and Pd(dppf)Cl2 (220 mg, 0.308 mmol) were added. The reaction mixture was heated at 100° C. and stirred for 16 h. The reaction was monitored by TLC; after completion of the reaction, the reaction mixture was filtered through celite pad and extracted with EtOAc. The organic layer weas dried over anhydrous Na₂SO₄, filtered and concentrated under reduced pressure. The crude material was purified by column chromatography (gradient=5-10% EtOAc in heptane to afford ethyl (E/Z)-3-(3-(1-((tert-butoxycarbonyl)(methyl)amino)butyl)phenyl)acrylate (1.0 g, 95%) as pale yellow liquid. LC-MS: m/z=262.2 [M-Boc+H]⁺

7: To a stirring solution of ethyl (E/Z)-3-(3-(1-((tert-butoxycarbonyl)(methyl)amino) butyl)phenyl)acrylate (2.5 g, 6.91 mmol) in ethanol (30 mL) were added NiCl₂·6H₂O (327 mg. 1.38 mmol) and NaBH₄ (578 mg, 15.23 mmol) at 0° C. The reaction mixture was stirred at room temperature for 16 h. The reaction was monitored by TLC; after completion of the reaction, the reaction mixture was quenched with water and extracted with EtOAc. The combined organic layers were dried over anhydrous Na₂SO₄, filtered and concentrated. The crude material was purified by column chromatography (gradient=5-10% EtOAc in heptane) to afford ethyl 3-(3-(1-((tert-butoxycarbonyl)(methyl)amino)butyl)phenylpropanoate (2.4 g, 95%) as yellow liquid. LC-MS: m/z=264.2 [M-tBu+H]⁺

8: Prepared following general procedure 7b. Obtained 1.5 g, 65% yield. LC-MS: m/z=334.2 [M−H]⁻

9: Prepared following general procedure 2a. Obtained 2.8 g.

10: Prepared following general procedure 6. Obtained 2.7 g. LCMS m/z=303.2 [M+H]⁺

Intermediate W-2: Prepared following general procedure 4. Obtained 1.3 g, 39% yield. LC-MS m/z=398.2 [M+H]⁺

### Intermediate W-3: (E/Z)-3-(1-(2-cyano-N-methyl-3-(thiazol-2-yl)acrylamido)butyl)benzoic Acid

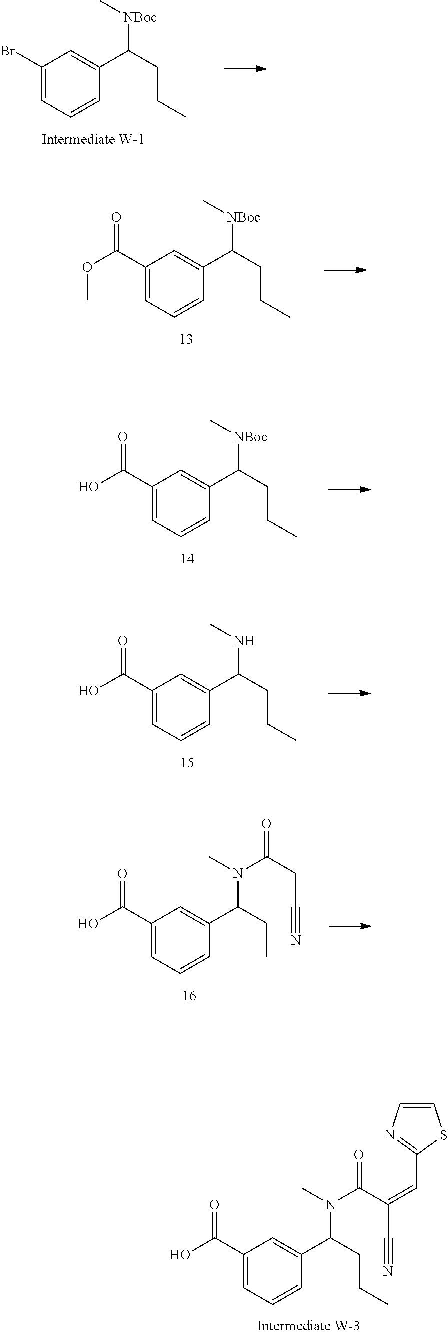 [SMILES: CCCC(c1cccc(Br)c1)N(C)C(=O)OC(C)(C)C ; CCCC(c1cccc(C(=O)OC)c1)N(C)C(=O)OC(C)(C)C ; CCCC(c1cccc(C(=O)O)c1)N(C)C(=O)OC(C)(C)C ; CCCC(NC)c1cccc(C(=O)O)c1 ; CCC(c1cccc(C(=O)O)c1)N(C)C(=O)CC#N ; CCCC(c1cccc(C(=O)O)c1)N(C)C(=O)/C(C#N)=C\c1nccs1]

13: To a solution of tert-butyl (1-(3-bromophenyl)butyl)(methyl)carbamate (4.0 g, 11.7 mmol) in MeOH (40 mL) were added Et₃N (6.7 mL, 48.9 mmol) and Pd(dppf)Cl₂ (1.0 g, 1.17 mmol) under CO gas pressure (5 kg) at room temperature. The resultant reaction mixture was heated at 90° C. for 16 h. The reaction was monitored by TLC; after completion of the reaction, the reaction mixture was filtered through celite bed and concentrated. The crude material was purified by column chromatography by eluting with 7-10% EtOAc in hexane to afford methyl 3-(1-((tert-butoxycarbonyl)(methyl)amino)butyl)benzoate (4.7 g, 85%, from two batches) as brown liquid. LC-MS: m/z=321.15 [M+H]⁺

14: Prepared following general procedure 7b. Obtained 3.8 g, 98% yield. LC-MS: m/z=306.2 [M−H]⁻

15: Prepared following general procedure 2a. Obtained 2 g, 74% yield. LC-MS: m/z=208.2 [M+H]⁺

16: Prepared following general procedure 6. Obtained 3.1 g, 75% yield. LC-MS: m/z=275.2 [M+H]⁺

Intermediate W-3: Prepared following general procedure 4. Obtained 2 g, 48% yield. LC-MS: m/z=370.2 [M+H]⁺

### Intermediate W-4: (E/Z)-3-(2-(1-(2-cyano-N-methyl-3-(thiazol-2-yl)acrylamido)butyl)phenyl)propanoic Acid

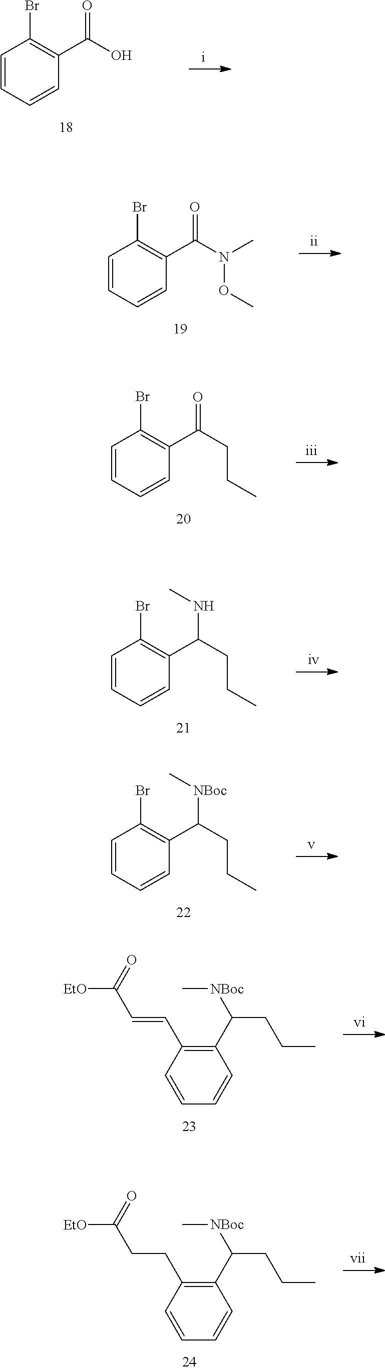

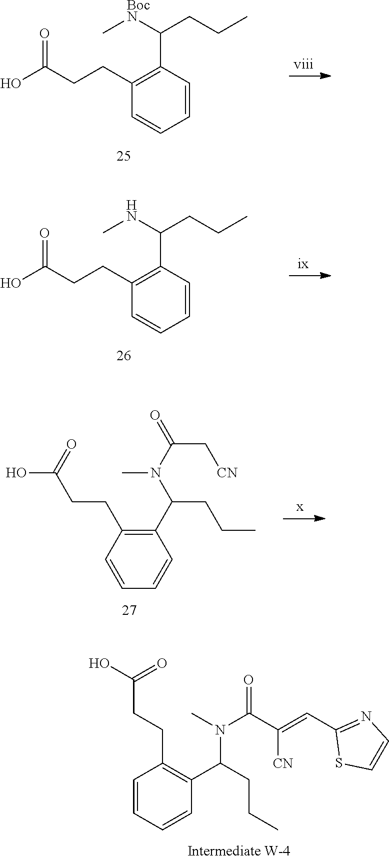 [SMILES: CCCC(c1ccccc1CCC(=O)O)N(C)C(=O)OC(C)(C)C ; CCCC(NC)c1ccccc1CCC(=O)O ; CCCC(c1ccccc1CCC(=O)O)N(C)C(=O)CC#N ; CCCC(c1ccccc1CCC(=O)O)N(C)C(=O)/C(C#N)=C/c1nccs1]

19: A stirring solution of 2-bromobenzoic acid (20.0 g, 99.50 mmol) in DMF (100 mL) was cooled to 0° C., then HATU (56.71 g, 149.25 mmol), DIPEA (50 mL, 298.50 mmol) and N,O-dimethylhydroxylamine hydrochloride (19.30 g, 199.00 mmol) were added. The reaction mixture was stirred at room temperature for 2 h. The reaction was monitored by TLC; after completion of the reaction, the reaction mixture was diluted with EtOAc and washed with water, followed by brine solution. The combined organic layers were dried over anhydrous sodium sulfate, filtered and concentrated. The crude compound was purified by column chromatography (gradient=20-25% EtOAc in hexane) to afford 2-bromo-N-methoxy-N-methylbenzamide (20.0 g, 83.3%) as yellow liquid. LC-MS: m/z=248.2 [M+H]⁺

20: A stirring solution of 2-bromo-N-methoxy-N-methylbenzamide (3.0 g, 12.29 mmol) in THF (30 mL) was cooled to −78° C. Then, propyl magnesium bromide (2.0 M in THF) (18 mL, 36.87 mmol) was added at room temperature. The reaction mixture was stirred at room temperature for 12 h. The reaction was monitored by TLC; after completion of the reaction, the reaction mixture was quenched with salt of ammonium chloride solution and extracted with EtOAc. The combined organic layers were dried over anhydrous Na₂SO₄, filtered and concentrated under reduced pressure. The crude material was purified by column chromatography by eluting with 10-15% EtOAc in hexane to afford 1-(2-bromophenyl)butan-1-one (1.5 g, 54%) as brown liquid.

LC-MS: m/z=227.0 [M+H]⁺

21: To a stirring solution of 1-(2-bromophenyl)butan-1-one (10.0 g, 44.033 mmol) in methanol (100 mL) was added MeNH₂ (2.0 M In THF) (70 mL, 132.15 mmol) and Ti(OiPr)₄ (18.76 g, 66.07 mmol) at 0° C. The reaction mixture was heated at 40° C. for 4 h. The reaction was monitored by TLC; after completion of the reaction, the reaction mixture was cooled to 0° C., then NaBH₄ (2.0 g, 52.86 mmol) was added portion-wise and the reaction mixture was stirred for 4 h. The reaction mixture was quenched with ice water and filtered through celite bed, and then concentrated reaction mixture was diluted with EtOAc and washed with water. The combined organic layers were dried over anhydrous Na₂SO₄, filtered and concentrated under reduced pressure. The obtained crude was purified by column chromatography (gradient=15-20% EtOAc in hexane) to afford 1-(2-bromophenyl)-N-methylbutan-1-amine (5.0 g, 50%) as brown liquid. LC-MS: m/z=244.0 [M+H]⁺

22: A stirring solution of 1-(2-bromophenyl)-N-methylbutan-1-amine (2.0 g, 8.26 mmol) in THF (20 mL) was cooled to 0° C., then Et₃N (2.4 mL, 16.52 mmol), DMAP (200 mg, 1.65 mmol) and di-tert-butyl dicarbonate (2.8 mL, 12.38 mmol) were added at room temperature. The reaction mixture was heated at 70° C. for 16 h. The reaction was monitored by TLC; after completion of the reaction, the reaction mixture was diluted with EtOAc and washed with water. The combined organic layers were dried over anhydrous Na₂SO₄, filtered and concentrated under reduced pressure. The obtained crude was purified by column chromatography (gradient=10-20% EtOAc in hexane) to afford tert-butyl (1-(2-bromophenyl)butyl)(methyl)carbamate (2.5 g, 88%) LC-MS: m/z=286.10 [M-Boc+H]⁺

23: To a stirring solution of tert-butyl (1-(2-bromophenyl)butyl)(methyl)carbamate (2.4 g, 7.01 mmol) in DMF (20 mL) were added Pd(dppf)·DCM adduct (570 mg, 0.701 mmol) and Et₃N (2.8 mL, 21.05 mmol) and the reaction mixture was degassed for 5 min. Then, ethyl acrylate (2.1 g, 21.05 mmol) was added. The resultant reaction mixture was heated at 140° C. for 4 h. The reaction was monitored by TLC; after completion of the reaction, the reaction mixture was diluted with ethyl acrylate and washed with water. The combined organic layers were dried over anhydrous Na₂SO₄, filtered and concentrated under reduced pressure. The crude material was purified by column chromatography (gradient=15-20% EtOAc in hexane) to afford ethyl (E/Z)-3-(2-(1-((tert-butoxycarbonyl)(methyl)amino)butyl)phenyl)acrylate (2.0 g, 79%) LC-MS: m/z=306.2 [M-56+H]⁺

24: To a stirring solution of NiCl₂ (130 mg, 0.553 mmol) in ethanol (15 ml) was added NaBH₄ (0.42 g, 11.06 mmol) at 0° C. Then, ethyl (E/Z)-3-(2-(1-((tert-butoxycarbonyl) (methyl)amino) butyl)phenyl)acrylate (2.0 g, 5.53 mmol) in ethanol (15 mL) was added slowly at room temperature. The reaction mixture was stirred at room temperature for 16 h before being concentrated in vacuo. The reaction mixture was diluted with EtOAc and washed with water and brine solution. The combined organic layers were dried over anhydrous Na₂SO₄, filtered and concentrated under reduced pressure to afford ethyl 3-(2-(1-((tert-butoxycarbonyl)(methyl)amino) butyl)phenyl)propanoate (2.0 g, crude). The crude was used for next step without further purification.

25: Prepared following general procedure 7. Obtained 1.6 g, LCMS m/z=358.1 [M+Na]⁺

26: Prepared following general procedure 2a. Obtained 1.2 g. LCMS m/z=236.2 [M+H]⁺

27: Prepared following general procedure 3. Obtained 1.7 g, 44% yield

Intermediate W-4: Prepared following general procedure 4. Obtained 0.85 g, 38% yield. LC-MS: m/z=398.3 [M+H]⁺

### Intermediate W-5: (E/Z)-3-(2-(2-cyano-3-(thiazol-2-yl)acryloyl)-1-methyl-1,2,3,4-tetrahydroisoquinolin-7-yl)propanoic Acid

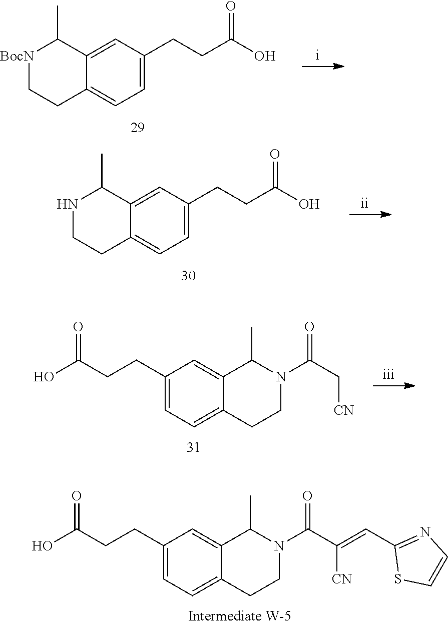 [SMILES: CC1c2cc(CCC(=O)O)ccc2CCN1C(=O)OC(C)(C)C ; CC1NCCc2ccc(CCC(=O)O)cc21 ; CC1c2cc(CCC(=O)O)ccc2CCN1C(=O)CC#N ; CC1c2cc(CCC(=O)O)ccc2CCN1C(=O)/C(C#N)=C/c1nccs1]

30: Prepared following general procedure 2, using starting material prepared according to WO2022/129925. Obtained 8.5 g. LCMS m/z=220.3 [M+H]⁺.

31: Prepared following general procedure 3. Obtained 3.2 g, 51% yield. LCMS m/z=285.2 [M−H]⁺.

Intermediate W-5: Prepared following general procedure 4. Obtained 1.7 g, 26% yield. ¹H-NMR (400 MHz, CD₃OD): δ 8.13 (d, 1H, J=3.0 Hz), 8.00 (d, 1H, J=2.9 Hz), 7.95 (brs, 1H), 7.11 (brs, 2H), 7.06 (s, 1H), 5.52-5.18 (m, 1H), 4.18-4.04 (m, 1H), 3.74-3.61 (m, 1H), 3.13 (brd, 1H, J=1.5 Hz), 2.92-2.81 (m, 3H), 2.56-2.54 (m, 2H), 1.62-1.49 (m, 3H). LCMS m/z=382.2 [M+H]⁺.

### Intermediate W-6: (E/Z)-2-(6-(2-aminoethoxy)-1-methyl-1,2,3,4-tetrahydroisoquinoline-2-carbonyl)-3-(thiazol-2-yl)acrylonitrile

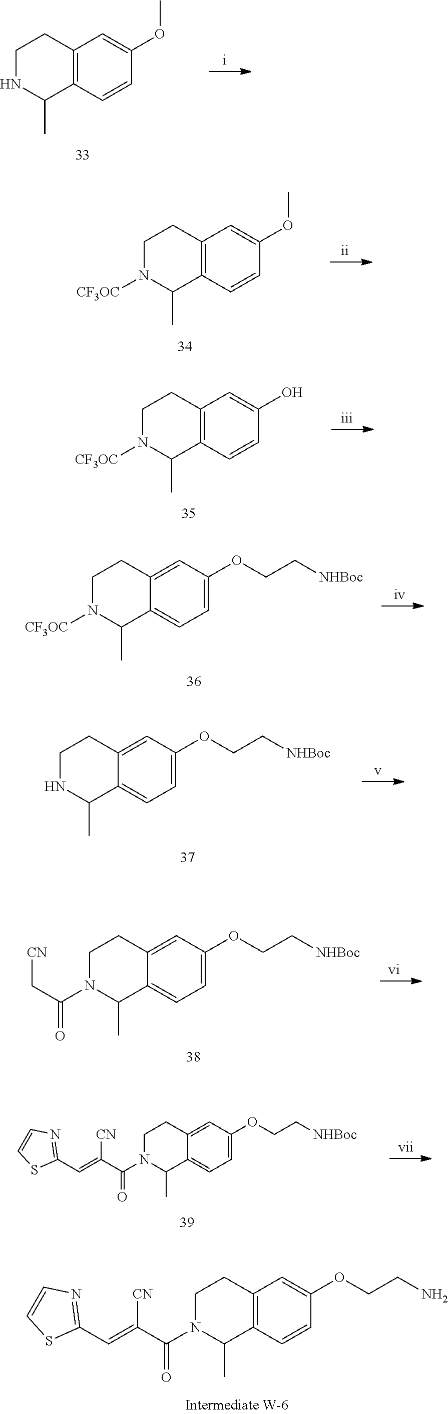

34: Prepared following general procedure 22 from 6-methoxy-1-methyl-1,2,3,4-tetrahydroisoquinoline (8.0 g, 45.13 mmol) (prepared as described in WO2022/129925). (7 g, 57% yield). LCMS m/z=274.2 [M+H]⁺

35: To a stirring solution of 2,2,2-trifluoro-1-(6-methoxy-1-methyl-3,4-dihydro-1H-isoquinolin-2-yl)ethanone (4.0 g, 14.63 mmol) in DCM (50 mL) was added BBr₃ (1 M in DCM, 21.9 mL, 21.9 mmol) at 0° C. under N2 atmosphere. The reaction mixture was stirred at RT for 2 h. before being quenched with MeOH and concentrated in vacuo. The resulting crude residue was diluted with ice water and extracted with EtOAc. The combined organic layers were dried over anhydrous Na₂SO₄, filtered and concentrated in vacuo to afford 2,2,2-trifluoro-1-(6-hydroxy-1-methyl-3,4-dihydro-1H-isoquinolin-2-yl)ethanone (3.5 g, 94% yield). LCMS m/z=260.1 [M+H]⁺

36: To a stirring solution of 2,2,2-trifluoro-1-(6-hydroxy-1-methyl-3,4-dihydro-1H-isoquinolin-2-yl)ethanone (3.5 g, 13.50 mmol) in toluene (50 mL) was added tert-butyl N-(2-hydroxyethyl)carbamate (2.61 g, 16.2 mmol) and cyanomethyl tributyl phosphorane (4.88 g, 20.25 mmol) at RT. The reaction mixture was stirred at 80° C. for 16 h before being concentrated in vacuo. The resulting crude residue was diluted with ice-water and extracted with DCM. The combined organic layers were dried over anhydrous Na₂SO₄, filtered and concentrated in vacuo to afford tert-butyl N-[2-[[1-methyl-2-(2,2,2-trifluoroacetyl)-3,4-dihydro-1H-isoquinolin-8-yl]oxy]ethyl]carbamate (4.0 g crude) as a white solid. The crude product was used in the next step without further purification. LCMS m/z=805.4 [2M+H]⁺

37: Prepared following general procedure 20. (4.0 g crude). The crude product was used in the next step without further purification. LCMS m/z=307.2 [M+H]⁺

38: Prepared following general procedure 3. Obtained 3.0 g, 61.6% yield. LCMS m/z=274.2 [M-Boc+H]⁺.

39: Prepared following general procedure 4. Obtained 1.5 g, 60% yield. LCMS m/z=469.3 [M+H]⁺

Intermediate W-6: Prepared following general procedure 2. After silica gel column chromatography (gradient=10% MeOH in DCM), obtained 340 mg, 87% yield. LCMS m/z=369.3 [M+H]⁺.

### Intermediate W-7: (E/Z)-2-(6-((5-aminopentyl)oxy)-1-methyl-1,2,3,4-tetrahydroisoquinoline-2-carbonyl)-3-(thiazol-2-yl)acrylonitrile

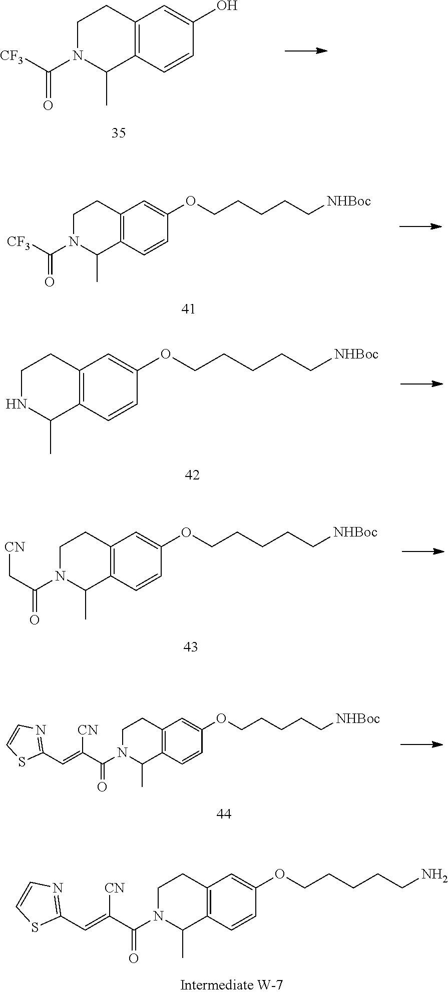 [SMILES: CC1c2ccc(O)cc2CCN1C(=O)C(F)(F)F ; CC1c2ccc(OCCCCCNC(=O)OC(C)(C)C)cc2CCN1C(=O)C(F)(F)F ; CC1NCCc2cc(OCCCCCNC(=O)OC(C)(C)C)ccc21 ; CC1c2ccc(OCCCCCNC(=O)OC(C)(C)C)cc2CCN1C(=O)CC#N ; CC1c2ccc(OCCCCCNC(=O)OC(C)(C)C)cc2CCN1C(=O)/C(C#N)=C/c1nccs1 ; CC1c2ccc(OCCCCCN)cc2CCN1C(=O)/C(C#N)=C/c1nccs1]

41: To a stirring solution of 2,2,2-trifluoro-1-(6-hydroxy-1-methyl-3,4-dihydro-1H-isoquinolin-2-yl)ethanone (35) (4.0 g, 15.43 mmol) in toluene (50 mL) was added tert-butyl N-(5-hydroxypentyl)carbamate (3.76 g, 18.51 mmol) and cyanomethyl tributyl phosphorane (5.57 g, 23.13 mmol) at RT. The reaction mixture was stirred at 80° C. for 16 h before being concentrated in vacuo. The resulting crude residue was diluted with water and extracted with DCM. The combined organic extracts were dried over anhydrous Na₂SO₄, filtered and concentrated in vacuo. The resulting crude material was purified by silica gel column chromatography (gradient=20% EtOAc in heptane) to afford tert-butyl N-[5-[[1-methyl-2-(2,2,2-trifluoroacetyl)-3,4-dihydro-1H-isoquinolin-6-yl]oxy]pentyl]carbamate (4.5 g, 65.7% yield). LCMS m/z=334.2 [M-Boc+H]⁺.

42: Prepared following general procedure 20. Obtained 3.0 g, 96% yield. LCMS m/z=349.4 [M+H]⁺.

43: Prepared following general procedure 3. Obtained 2.0 g. 48% yield. LCMS m/z=316.3 [M-Boc+H]⁺.

44: Prepared following general procedure 4. Obtained 2.2 g, 59% yield. LCMS m/z=411.3 [M+H]⁺

Intermediate W-7: Prepared following general procedure 2. After silica gel column chromatography (gradient=10% MeOH in DCM), obtained 400 mg, 98% yield. LCMS m/z=411.2 [M+H]⁺.

### Intermediate W-8: (E/Z)-2-(2-((2-(2-cyano-3-(thiazol-2-yl)acryloyl)-1-methyl-1,2,3,4-tetrahydroisoquinolin-7-yl)oxy)ethoxy)acetic Acid

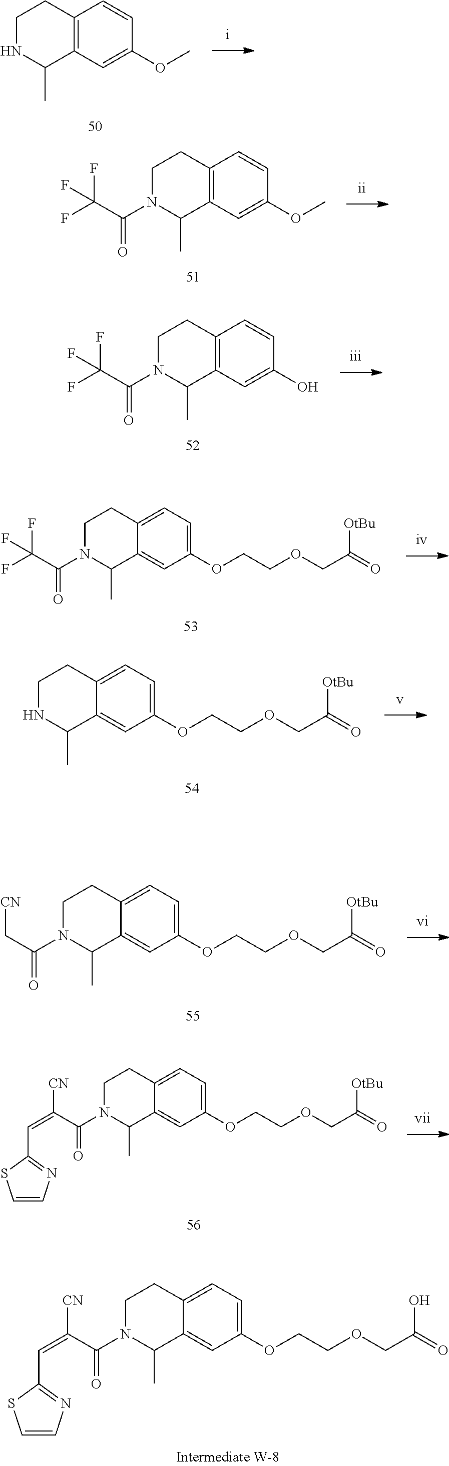

50: Prepared as described in WO2021/71843, para 346-349;

51: To a stirring solution of 7-methoxy-1-methyl-1,2,3,4-tetrahydroisoquinoline (1.4 g, 7.89 mmol) in DCM (15 mL) was added TEA (3.29 mL, 23.67 mmol) drop wise at 0° C. followed by an addition of TFAA (1.64 mL, 11.84 mmol) under nitrogen atmosphere. The reaction mixture was allowed to warm to room temperature and stirred for 6 h. The reaction mixture was quenched with ice cooled water and extracted with ethyl acetate. The combined organic layers were concentrated under reduced pressure to afford 2,2,2-trifluoro-1-(7-methoxy-1-methyl-3,4-dihydroisoquinolin-2(1H)-yl)ethan-1-one (2.0 g, 93%) as pale brown solid, which was used as such in next step without further purification. LCMS m/z=274.48 [M+H]⁺

52: To a stirring solution of 2,2,2-trifluoro-1-(7-methoxy-1-methyl-3,4-dihydroisoquinolin-2(1H)-yl)ethan-1-one (0.5 g, 1.83 mmol) in DCM (5 mL) was added BBr₃ (2.75 mL, 1 M in DCM) dropwise at 0° C. and stirred for 2 h under nitrogen atmosphere. The reaction mixture was quenched with methanol and diluted with Ice cooled water then extracted with DCM. The combined organic layers were concentrated under reduced pressure to afford 2,2,2-trifluoro-1-(7-hydroxy-1-methyl-3,4-dihydroisoquinolin-2(1H)-yl)ethan-1-one (0.4 g, 84%) as pale brown solid. LCMS m/z=260.38 [M+H]⁺

53: To a stirring solution of 2,2,2-trifluoro-1-(7-hydroxy-1-methyl-3,4-dihydroisoquinolin-2(1H)-yl)ethan-1-one (0.4 g, 1.83 mmol) in toluene (4 mL) was added tert-butyl 2-(2-hydroxyethoxy)acetate (0.3 g, 1.85 mmol) followed by cyanomethylenetributylphosphorane (0.63 g, 2.31 mmol) at room temperature. The reaction mixture was heated to 80° C. and stirred for 1 h. The reaction mixture was evaporated and washed with water then extracted with ethyl acetate. The combined organic layers were concentrated under reduced pressure to afford tert-butyl 2-(2-((1-methyl-2-(2,2,2-trifluoroacetyl)-1,2,3,4-tetrahydroisoquinolin-7-yl)oxy)ethoxy)acetate (0.23 g, 36%) as pale brown solid. This material was used for next step without further purification. LCMS m/z=435.59 [M+18]⁺

54: Prepared following general procedure 20. Obtained 0.018 g 11% yield as pale brown solid. LCMS m/z=322.1 [M+H]⁺

55: Prepared following general procedure 6. Obtained 0.16 g, 39% yield. LCMS m/z=387.2 (M−H)⁻

56: Prepared following general procedure 4. Obtained 0.15 g, 81% yield. LCMS m/z=484.3 [M+H]⁺

Intermediate W-8: Prepared following general procedure 7. Obtained 0.12 g, 91% yield. LCMS m/z=428.23 [M+H]⁺

### Intermediate W-9: (E/Z)-5-((2-(2-cyano-3-(thiazol-2-yl)acryloyl)-1-methyl-1,2,3,4-tetrahydroisoquinolin-7-yl)oxy)pentanoic Acid

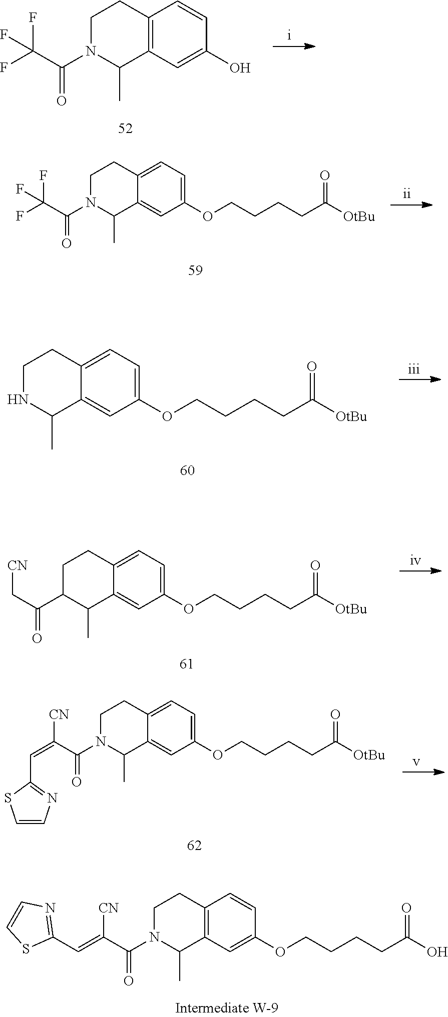 [SMILES: CC1c2cc(O)ccc2CCN1C(=O)C(F)(F)F ; CC1c2cc(OCCCCC(=O)OC(C)(C)C)ccc2CCN1C(=O)C(F)(F)F ; CC1NCCc2ccc(OCCCCC(=O)OC(C)(C)C)cc21 ; CC1c2cc(OCCCCC(=O)OC(C)(C)C)ccc2CCC1C(=O)CC#N ; CC1c2cc(OCCCCC(=O)OC(C)(C)C)ccc2CCN1C(=O)/C(C#N)=C\c1nccs1 ; CC1c2cc(OCCCCC(=O)O)ccc2CCN1C(=O)/C(C#N)=C/c1nccs1]

59: To a stirring solution of 2,2,2-trifluoro-1-(7-hydroxy-1-methyl-3,4-dihydroisoquinolin-2(1H)-yl)ethan-1-one (52) (0.5 g, 1.93 mmol) in DMF (5 ml) was added K₂CO₃ (0.8 g, 5.79 mmol) followed by tert-butyl 5-bromopentanoate (0.59 g, 2.31 mmol) at room temperature. The reaction mixture was heated to 80° C. and stirred for 16 h. The reaction mixture was diluted with water and extracted with ethyl acetate. The combined organic layers were dried over anhydrous Na₂SO₄, filtered and concentrated under reduced pressure. The obtained crude was purified by medium pressure liquid chromatography (gradient=20-30% ethyl acetate in heptane) to afford tert-butyl 5-((1-methyl-2-(2,2,2-trifluoroacetyl)-1,2,3,4-tetrahydroisoquinolin-7-yl)oxy)pentanoate (0.45 g, 56%) as pale brown solid. LCMS m/z=360.34 [M−56+H]⁺.

60: Prepared following general procedure 20. Obtained 0.32 g, 92% yield as colorless liquid. LCMS m/z=320.3 [M+H]+

61: Prepared following general procedure 6. Obtained 0.37 g, 94% yield. LCMS m/z=385.1 [M−H]⁻

62: Prepared following general procedure 4. Obtained 0.32 g, 69% yield. LCMS m/z=482.1 [M+H]+

Intermediate W-9: Prepared following general procedure 7a Obtained 0.16 g, 57% yield. LCMS m/z=426.28 [M+H]+

### Intermediate W-10: (E/Z)-2-(7-(2-aminoethoxy)-1-methyl-1,2,3,4-tetrahydroisoquinoline-2-carbonyl)-3-(thiazol-2-yl)acrylonitrile

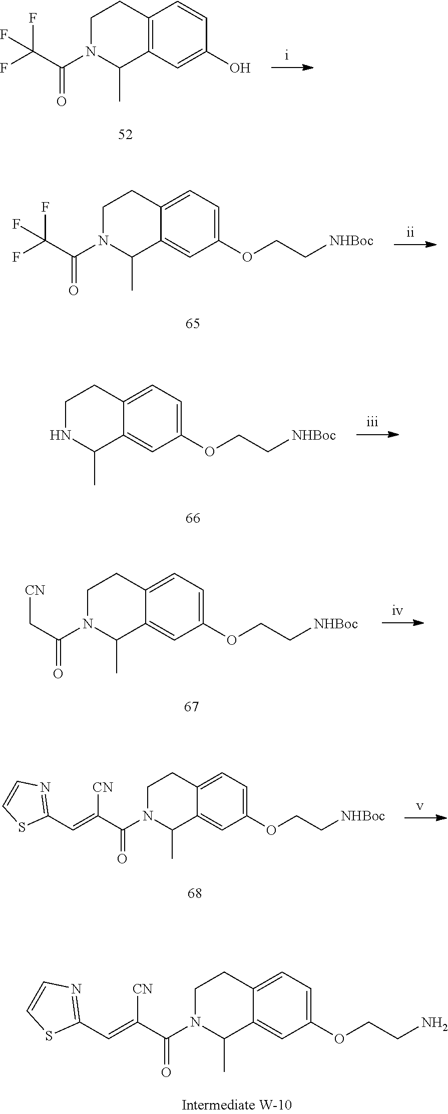 [SMILES: CC1c2cc(O)ccc2CCN1C(=O)C(F)(F)F ; CC1c2cc(OCCNC(=O)OC(C)(C)C)ccc2CCN1C(=O)C(F)(F)F ; CC1NCCc2ccc(OCCNC(=O)OC(C)(C)C)cc21 ; CC1c2cc(OCCNC(=O)OC(C)(C)C)ccc2CCN1C(=O)CC#N ; CC1c2cc(OCCNC(=O)OC(C)(C)C)ccc2CCN1C(=O)/C(C#N)=C/c1nccs1 ; CC1c2cc(OCCN)ccc2CCN1C(=O)/C(C#N)=C/c1nccs1]

65: To a stirring solution of 2,2,2-trifluoro-1-(7-hydroxy-1-methyl-3,4-dihydroisoquinolin-2(1H)-yl)ethan-1-one (52) (4 g, 15.44 mmol) and tert-butyl (2-hydroxyethyl)carbamate (2.9 g, 18.53) in toluene (40 mi) was added TBPA (5.6 g, 23.1 mmol) in sealed tube at 80° C. for 16 h. The reaction was monitored by TLC; after completion, the reaction mixture was diluted with water and extracted with EtOAc. The combined organic layers were dried over anhydrous Na₂SO₄, filtered and concentrated under reduced pressure The crude material was purified by medium pressure liquid column chromatography to afford tert-butyl (2-((1-methyl-2-(2,2,2-trifluoroacetyl)-1,2,3,4-tetrahydroisoquinolin-7-yl)oxy)ethyl)carbamate (6.0 g, 96%). LC-MS: m/z=347.0 [M-56+H]⁺

66: Prepared following general procedure 20. Obtained 5 g, crude. LC-MS: m/z=307.2 [M+H]⁺

67: Prepared following general procedure 6. Obtained 3.7 g, 68% yield. LC-MS: m/z=372.2 [M−H]⁻

68: Prepared following general procedure 4. Obtained 3.5 g. 76% yield. LC-MS: m/z=467.2 [M−H]⁻

Intermediate W-10: Prepared following general procedure 13. Obtained 0.75 g, 64% yield. LC-MS: m/z=369.2 [M+H]⁺

### Intermediate W-11: (E/Z)-2-[5-(2-aminoethoxy)-1-methyl-3,4-dihydro-1H-isoquinoline-2-carbonyl]-3-thiazol-2-yl-prop-2-enenitrile

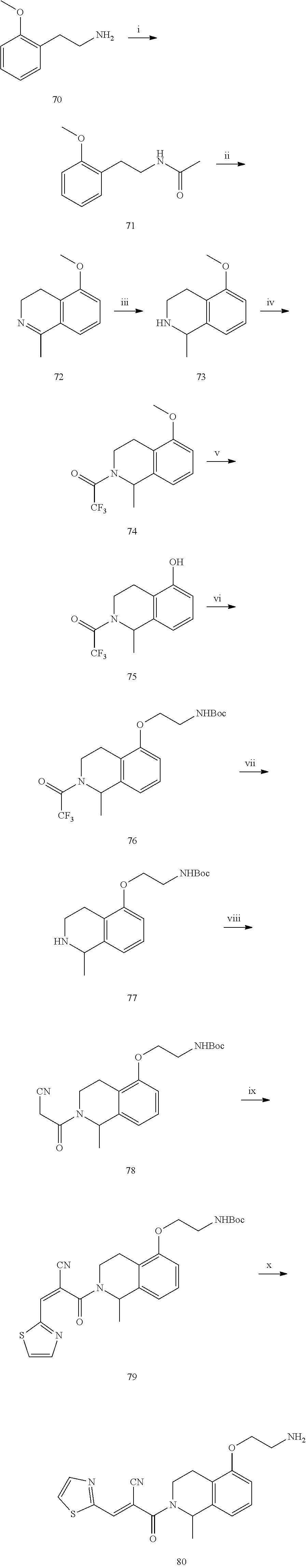

71: To a stirring solution of 2-(2-methoxyphenyl)ethanamine (15.0 g, 99.20 mmol) in CH₂Cl₂ (150 mL) was added at 0° C. under inert atmosphere Et₃N (34.3 mL, 339.35 mmol) and acetic anhydride (14.0 mL, 138.87 mmol) was added dropwise. The reaction mixture was brought to room temperature and stirred for 3 h. The reaction was monitored by TLC; after completion, the reaction mixture was quenched with cold water and extracted with CH₂Cl₂ (2×250 mL). The combined organic layers were dried over anhydrous Na₂SO₄, filtered and concentrated under reduced pressure to afford N-[2-(2-methoxyphenyl)ethyl]acetamide (18.8 g crude) as colorless oil. LC-MS: m/z=194.2 [M+H]⁺

72: To a stirring solution of N-[2-(2-methoxyphenyl)ethyl]acetamide (16.0 g, 82.79 mmol) in toluene (150 mL) were added POCl₃ (31 mL, 202.19 mmol) and P₂O₅ (30 g, 107.77 mmol) at 0° C. The reaction mixture was under reflux for 16 h. The reaction was monitored by TLC; after completion, the reaction mixture was cooled to 0° C. and basified with 25% NaOH solution and extracted with EtOAc. The combined organic layers were dried over anhydrous Na₂SO₄, filtered and concentrated under reduced pressure. The crude material was purified by column chromatography by using 40% EtOAc in heptane to afford 5-methoxy-1-methyl-3,4-dihydroisoquinoline (2.07 g, 14.3%) as yellow oil. LC-MS: m/z=176.2 [M+H]⁺

73: To a stirring solution of 5-methoxy-1-methyl-3,4-dihydroisoquinoline (2.07 g, 11.81 mmol) in ethanol (20 mL) 0° C. under inert atmosphere was added NaBH₄ (1.2 g, 35.44 mmol) portion wise. The reaction mixture was allowed to warm from 0° C. to room temperature and stirred for 16 h. The reaction was monitored by TLC; after completion, the reaction mixture was quenched with ice water and concentrated under reduced pressure and extracted with EtOAc (2×30 mL) The combined organic layers were dried over anhydrous Na₂SO₄, filtered and concentrated under reduced pressure to afford 5-methoxy-1-methyl-1,2,3,4-tetrahydroisoquinoline (1.9 g crude) as yellow solid. The resultant crude was used for next step without further purification. LC-MS: m/z=178.2 [M+H]⁺

74: Prepared by following general procedure 22. Obtained 4.5 g, 74.9%. LC-MS: m/z=274.0 [M+H]⁺

75: To a stirring solution of 2,2,2-trifluoro-1-(5-methoxy-1-methyl-3,4-dihydro-1H-isoquinolin-2-yl)ethenone (4.2 g, 15.37 mmol) in CH₂Cl₂ (45 mL), BBr₃ (23 mL, 1M in CH₂Cl₂) was added dropwise at 0° C. and stirred for 2 h. The reaction was monitored by TLC; after completion, the reaction mixture was quenched with MeOH and concentrated under reduced pressure. The crude was diluted with ice water (100 ml) and extracted with EtOAc (2×120 mL). The combined organic layers were dried over anhydrous Na₂SO₄, filtered and concentrated under reduced pressure. The crude material was purified by column chromatography by using 10% EtOAc in heptane to afford 2,2,2-trifluoro-1-(5-hydroxy-1-methyl-3,4-dihydro-1H-isoquinolin-2-yl)ethenone (3.4 g, 85.4%). LC-MS: m/z=260.2 [M+H]⁺

76: To a stirring solution of 2,2,2-trifluoro-1-(5-hydroxy-1-methyl-3,4-dihydro-1H-isoquinolin-2-yl)ethenone (3.4 g, 13.12 mmol) in toluene (35 mL) tert-butyl N-(2-hydroxyethyl)carbamate (2.53 g, 15.73 mmol) and TBPA (4.74 g, 19.69 mmol) were added at room temperature. The reaction mixture was stirred at 80° C. for 16 h. The reaction was monitored by TLC; after completion, the reaction mixture was concentrated under reduced pressure. The crude material was purified by column chromatography by using 30% EtOAc in heptane to afford tert-butyl N-[2-[[1-methyl-2-(2,2,2-trifluoroacetyl)-3,4-dihydro-1H-isoquinolin-5-yl]oxy]ethyl]carbamate (5.27 g, 98%) as white solid. LC-MS: m/z=347.2 [M-56+H]⁺

77: Prepared by following procedure 20/32. Obtained 4.0 g, 99%) as pale yellow oily compound. LC-MS: m/z=307.3 [M-56+H]⁺

78: Prepared following general procedure 3. Obtained 4.33 g, 88% yield. LC-MS: m/z=318.0 [M−56+H]⁺

79: Prepared following general procedure 4. Obtained 2.5 g, 60% yield.

Intermediate W-11: Prepared following general procedure 6. Obtained 0.12 g, 91% yield. LC-MS: m/z=369.1 [M+H]⁺.

### Intermediates W-12 to W-14

### 1-(7-bromo-1-methyl-3,4-dihydroisoquinolin-2(1H)-yl)-2,2,2-trifluoroethan-1-one (Intermediate W-12)

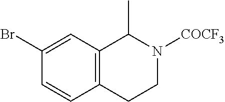

By general procedure 22 from 7-bromo-1-methyl-1,2,3,4-tetrahydroisoquinoline as described in WO2022/129925. Obtained 1.2 g, 71% yield.

### Benzyl 7-bromo-1-methyl-3,4-dihydroisoquinoline-2(1H)-carboxylate (Intermediate W-13)

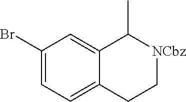 [SMILES: CC1c2cc(Br)ccc2CCN1C(=O)OCc1ccccc1]

By general procedure 22 from 7-bromo-1-methyl-1,2,3,4-tetrahydroisoquinoline as described in WO2022/129925 using CbzCl (1.3 eq) instead of trifluoroacetate anhydride. Obtained 830 mg, 51% yield.

### tert-butyl 7-bromo-1-methyl-3,4-dihydroisoquinoline-2(1H)-carboxylate (Intermediate W-14)

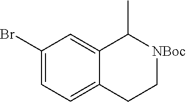 [SMILES: CC1c2cc(Br)ccc2CCN1C(=O)OC(C)(C)C]

Synthesised as described in WO2022/129925

### Intermediates W-15 and W-18

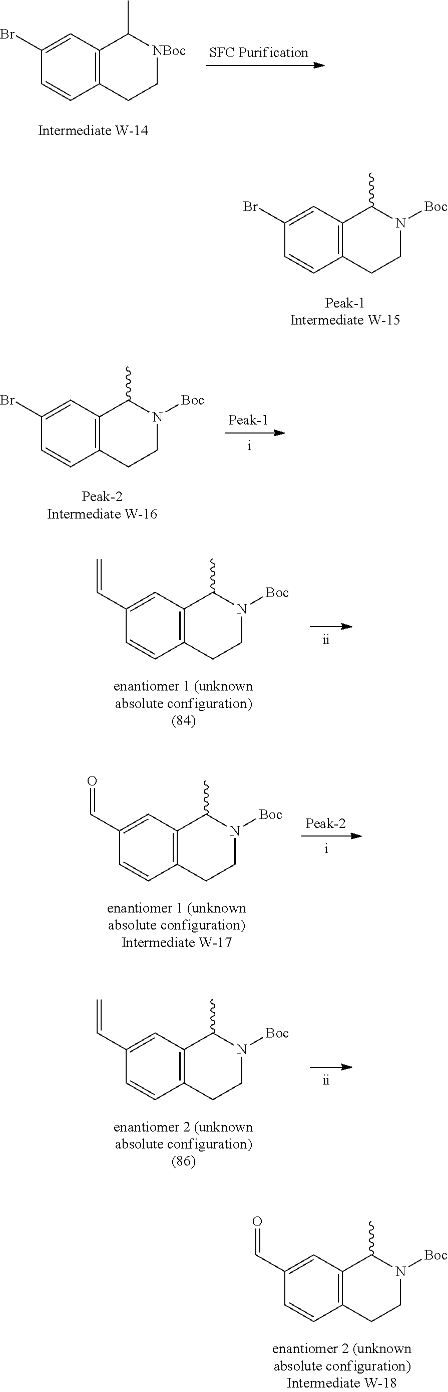

Racemic tert-butyl 7-bromo-1-methyl-3,4-dihydroisoquinoline-2(1H)-carboxylate (20 g) was purified by chiral SFC:

| | |
|---|---|
| Instrument | PIC 22-027 |
| Column | WHELK RR-(250*30 mm), 5 μm |
| Mobile Phase | CO₂: 0.5% isopropylamine in MeOH:MeCN [90:10] |
| Flow rate | 120 mL/min |
| Back pressure | 100 bar |
| Wavelength | 220 nm |
| Cycle time | 6 min |

Isolated Peak-1: 9.1 g, 46% yield. LCMS m/z=228.2 [M-Boc]⁺ and Peak-2: 9 g. 45% yield. LCMS m/z=226.2 [M-Boc]⁺

### Enantiomer of tert-butyl 1-methyl-7-vinyl-3,4-dihydroisoquinoline-2(1H)-carboxylate (84)

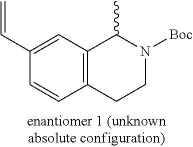 [SMILES: C=Cc1ccc2c(c1)C(C)N(C(=O)OC(C)(C)C)CC2]

By general procedure 30. Obtained 1.5 g, 68% yield. LCMS m/z=218.2 [M−56]⁺.

### Enantiomer 1 of tert-butyl 7-formyl-1-methyl-3,4-dihydroisoquinoline-2(1H)-carboxylate (Intermediate W-16)

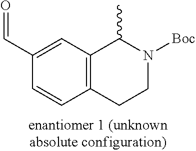 [SMILES: CC1c2cc(C=O)ccc2CCN1C(=O)OC(C)(C)C]

By general procedure 31. Obtained 1 g, 66% yield. LCMS m/z=176.3 [M-Boc]⁺.

### Enantiomer 2 of tert-butyl 1-methyl-7-vinyl-3,4-dihydroisoquinoline-2(1H)-carboxylate (86)

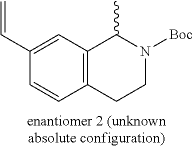 [SMILES: C=Cc1ccc2c(c1)C(C)N(C(=O)OC(C)(C)C)CC2]

By general procedure 30. Obtained 1.5 g, 67% yield. LCMS m/z=218.2 [M-56]⁺.

### Enantiomer 2 of tert-butyl 7-formyl-1-methyl-3,4-dihydroisoquinoline-2(1H)-carboxylate (Intermediate W-17)

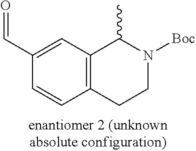 [SMILES: CC1c2cc(C=O)ccc2CCN1C(=O)OC(C)(C)C]

By general procedure 31. Obtained 900 mg, 64% yield. LCMS m/z=176.3 [M-Boc]⁺.

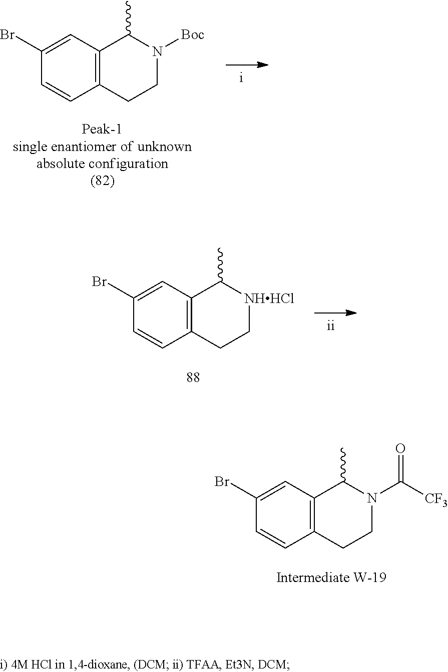

### Enantiomer 1 of 7-bromo-1-methyl-1,2,3,4-tetrahydroisoquinoline (88)

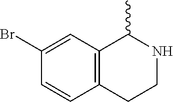

By general procedure 2 using 1,4-dioxane in HCl in DCM. Obtained 1.6 g (crude). LCMS m/z=263.3 [M+H]⁺.

### Enantiomer of 1-(7-bromo-1-methyl-3,4-dihydroisoquinolin-2(1H)-yl)-2,2,2-trifluoroethan-1-one (Intermediate W-19)

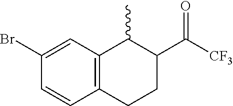 [SMILES: CC1c2cc(Br)ccc2CCC1C(=O)C(F)(F)F]

By general procedure 22. Obtained 1.94 g, 92% yield.

### General Procedure—for Preparation of Bromo THIQs

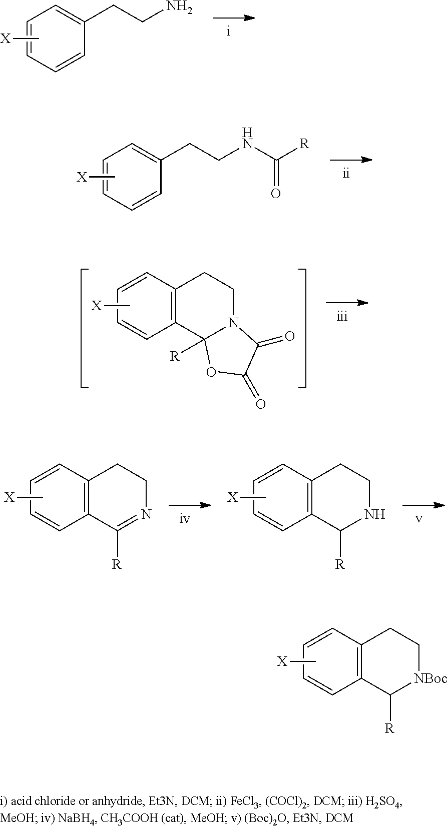

The following examples were prepared by general procedure BR-THIQ 1

| Starting | | | | |
|---|---|---|---|---|
| material | | | | LCMS m/z |
| name | Product name | Reagent | Product Structure | [M + H]⁺ |
| 2-(2- bromophenyl) ethan-1-amine | N-(2- bromophenethyl)propion amide | Propionyl chloride | 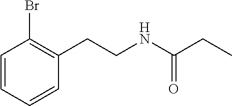 | 256.3/93% |
| 2-(2- bromophenyl) ethan-1-amine | N-(2-bromophenethyl)- 2,2-difluoroacetamide | Difluoroacetic anhydride | 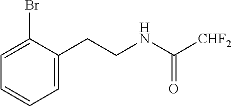 [SMILES: O=C(NCCc1ccccc1Br)C(F)F] | 256.0/58% |
| 2-(2- bromophenyl) ethan-1-amine | N-(2-bromophenethyl)- 2-methoxyacetamide | 2-methoxyacetyl chloride | 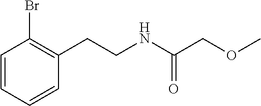 | 272.2/97% |
| 2-(2- bromophenyl) ethan-1-amine | (2-methylpentan-2- yl)benzene | butyryl chloride | 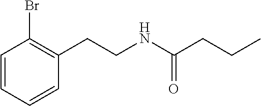 | 270.2/97% |

General procedure BR-THIQ 1

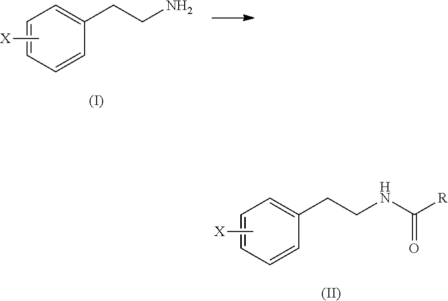

To a stirred solution of (I) (1 eq) in DCM was added Et3N (1.5 eq) followed by an acid chloride or anhydride (1.1 eq) at 0° C. The reaction was warmed to RT and stirred for 16 h. The reaction mixture was cooled to −78° C. and iron(III) chloride (3 eq) was added. The reaction mixture was quenched with cold water and extracted with DCM. The combined organic layers were dried over anhydrous Na₂SO₄, filtered and concentrated in vacuo to afford the desired product (II). The resulting crude solid was without further purification.

The following examples were prepared by general procedure BR-THIQ 2

| Starting | | | |
|---|---|---|---|
| material | | | LCMS m/z |
| name | Product name | Product Structure | [M + H]⁺ |
| N-(2- bromophenethyl) propionamide | 7-bromo-10b-ethyl- 6,10b-dihydro-5H- oxazolo[2,3- a]isoquinoline-2,3-dione | 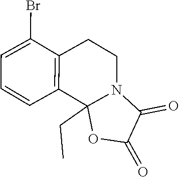 [SMILES: CCC12OC(=O)C(=O)N1CCc1c(Br)cccc12] | No ionisation |
| N-(2- bromophenethyl) formamide | 7-bromo-6,10b-dihydro- 5H-oxazolo[2,3- a]isoquinoline-2,3-dione | 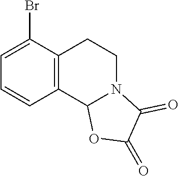 [SMILES: O=C1OC2c3cccc(Br)c3CCN2C1=O] | No ionisation |
| N-(2- bromophenethyl)- 2- methoxyacetamide | 7-bromo-10b- (methoxymethyl)-6,10b- dihydro-5H-oxazolo[2,3- a]isoquinoline-2,3-dione | 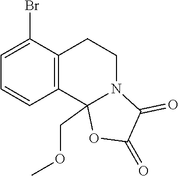 [SMILES: COCC12OC(=O)C(=O)N1CCc1c(Br)cccc12] | 328.0/98% |
| (2- methylpentan- 2-yl)benzene | 7-bromo-10b-propyl- 6,10b-dihydro-5H- oxazolo[2,3- a]isoquinoline-2,3-dione | 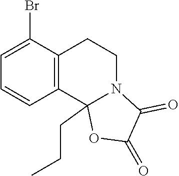 [SMILES: CCCC12OC(=O)C(=O)N1CCc1c(Br)cccc12] | No ionisation/ 94% |

### General Procedure BR-THIQ 2

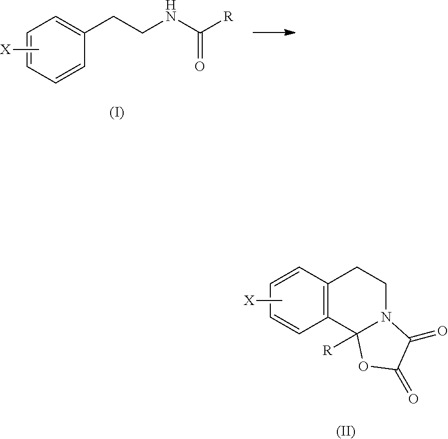

To a stirred solution of (I) (1 eq) in DCM was added oxalyl chloride (6 eq) at 0° C. The reaction was warmed to RT and stirred for 2 h. The reaction mixture was cooled to −78° C. and iron(III) chloride (3 eq) was added. The reaction mixture was stirred overnight at RT before being filtered through celite and washed with DCM. The filterate was concentrated in vacuo to afford crude product (II).

The following examples were prepared by general procedure BR-THIQ 3.

| Starting | | | | |
|---|---|---|---|---|
| material | | | | LCMS m/z |
| name | Product name | Product Structure | Conditions | [M + H]⁺/Yield |
| 7-bromo-10b- ethyl-6,10b- dihydro-5H- oxazolo[2,3- a]isoquinoline- 2,3-dione | 5-bromo-1-ethyl-3,4- dihydroisoquinoline | 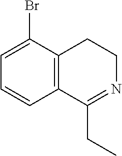 | BR-THIQ 3 | 240.2/53% |
| 7-bromo- 6,10b-dihydro- 5H- oxazolo[2,3- a]isoquinoline- 2,3-dione | 5-bromo-3,4- dihydroisoquinoline | 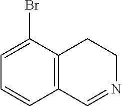 [SMILES: Brc1cccc2c1CCN=C2] | BR-THIQ 3 | 212.0/used without purification |
| N-(2- bromophenethyl)- 2,2- difluoroacetamide | 5-bromo-1- (difluoromethyl)-3,4- dihydroisoquinoline | 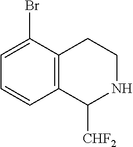 [SMILES: FC(F)C1NCCc2c(Br)cccc21] | BR-THIQ 4 | 262.2/89% |
| 7-bromo-10b- (methoxymethyl)- 6,10b- dihydro-5H- oxazolo[2,3- a]isoquinoline- 2,3-dione | 5-bromo-1- (methoxymethyl)-3,4- dihydroisoquinoline | 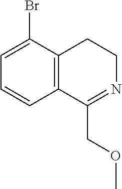 | BR-THIQ 3 | 254.0/8% |
| 7-bromo-10b- (methoxymethyl)- 6,10b- dihydro-5H- oxazolo[2,3- a]isoquinoline- 2,3-dione | 5-bromo-1-methoxy-1- (methoxymethyl)- 1,2,3,4- tetrahydroisoquinoline | 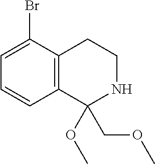 [SMILES: COCC1(OC)NCCc2c(Br)cccc21] | BR-THIQ 3 (isolated as a byproduct of the reaction) | 286.0/5% |
| 7-bromo-10b- propyl-6,10b- dihydro-5H- oxazolo[2,3- a]isoquinoline- 2,3-dione | 5-bromo-1-propyl-3,4- dihydroisoquinoline | 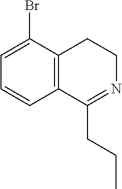 | BR-THIQ 3 | 254.2/82% |

### General Procedure BR-THIQ 3

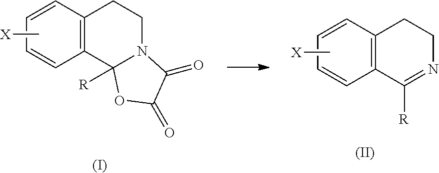

To a stirred solution of (1) (1 eq) in MeOH was added H₂SO₄ (7 eq) dropwise at RT. The reaction mixture was stirred 18 h at 80° C. before being concentrated in vacuo. The resulting residue was quenched with ice-cold water and the pH was adjusted using aqueous ammonia solution (30%) until pH=10. The resulting solution was extracted with DCM and the combined organic layers were dried over sodium sulphate, filtered and concentrated in vacuo to afford imine (II).

### General Procedure BR-THIQ 4

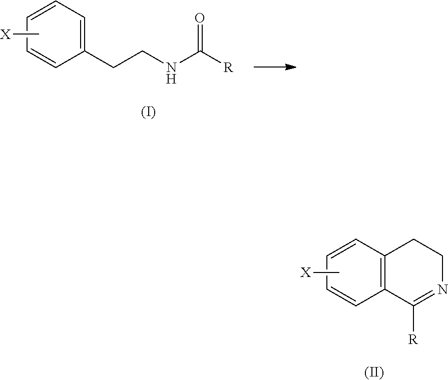

A stirred solution of (I) (1 eq) and 2-chloropyridine (1.1 eq) in DCM was cooled to −78° C. then trifluoromethanesulfonic anhydride (1.1 eq) was added dropwise over 10 min. The reaction mixture was stirred at 0° C. for 1 h before being heated to 80° C. The progress of reaction was monitored by LCMS. The reaction mixture was cooled to RT and Et3N (3 eq) was added. The reaction mixture was quenched with ice-water and extracted with DCM. The combined organic layer was dried with sodium sulphate, filtered and concentrated in vacuo to afford imine (II)

The following examples were prepared by general procedure BR-THIQ 5.

| Starting | | | |
|---|---|---|---|
| material | | | LCMS m/z |
| name | Product name | Product Structure | [M + H]⁺/Yield |
| 5-bromo-1- ethyl-3,4- dihydroisoquinoline | 5-bromo-1-ethyl-1,2,3,4- tetrahydroisoquinoline | 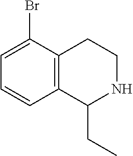 | 240.2/64% |
| 5-bromo-1- (difluoromethyl)- 3,4- dihydroisoquinoline | 5-bromo-1- (difluoromethyl)-1,2,3,4- tetrahydroisoquinoline | 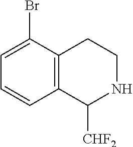 [SMILES: FC(F)C1NCCc2c(Br)cccc21] | 264.2/66% |
| 5-bromo-1- (methoxymethyl)- 3,4- dihydroisoquinoline | 5-bromo-1- (methoxymethyl)- 1,2,3,4- tetrahydroisoquinoline | 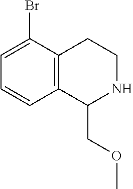 | 256.1/95% |
| 5-bromo-1- propyl-3,4- dihydroisoquinoline | 5-bromo-1-propyl- 1,2,3,4- tetrahydroisoquinoline | 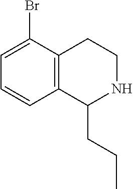 | 254.2/9% |

### General Procedure BR-THIQ 5

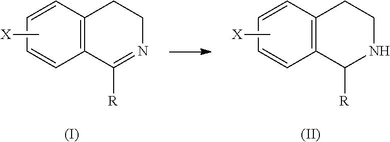

To a stirred solution of imine (I 1eq) in MeOH was added catalytic acetic acid, followed by NaBH₄ (2 eq) portionwise at 0° C. The reaction mixture was stirred at RT for 16 h before being concentrated in vacuo. The resulting residue was diluted in water and extracted with DCM. The organic layer was dried over sodium sulphate, filtered and concentrated in vacuo to afford amine (II).

The following examples were prepared by general procedure BR-THIQ 6.

| Starting | | | |
|---|---|---|---|
| material | | | LCMS m/z |
| name | Product name | Product Structure | [M + H]⁺/Yield |
| 5-bromo-1- ethyl-1,2,3,4- tetrahydroisoquinoline | tert-butyl 5-bromo-1- ethyl-3,4- dihydroisoquinoline- 2(1H)-carboxylate | 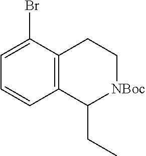 | 242.2 [M + H − Boc]+/59% |
| 5-bromo-1- (difluoromethyl)- 1,2,3,4- tetrahydroisoquinoline | tert-butyl 5-bromo-1- (difluoromethyl)-3,4- dihydroisoquinoline- 2(1H)-carboxylate | 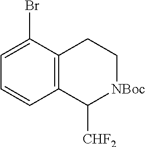 [SMILES: CC(C)(C)OC(=O)N1CCc2c(Br)cccc2C1C(F)F] | 264.2 [M + H − Boc]⁺/58% |
| 5-bromo-1- (methoxymethyl)- 1,2,3,4- tetrahydroisoquinoline | tert-butyl 5-bromo-1- (methoxymethyl)-3,4- dihydroisoquinoline- 2(1H)-carboxylate | 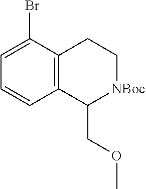 | 298.2 [M + H − tBu]⁺/73% |
| 5-bromo-1- methoxy-1- (methoxymethyl)- 1,2,3,4- tetrahydroisoquinoline | tert-butyl 5-bromo-1- methoxy-1- (methoxymethyl)-3,4- dihydroisoquinoline- 2(1H)-carboxylate | 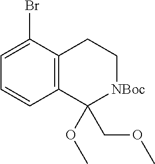 [SMILES: COCC1(OC)c2cccc(Br)c2CCN1C(=O)OC(C)(C)C] | 286.2 [M + H − Boc]⁺/57% |
| 5-bromo-1- propyl-1,2,3,4- tetrahydroisoquinoline | tert-butyl 5-bromo-1- propyl-3,4- dihydroisoquinoline- 2(1H)-carboxylate | 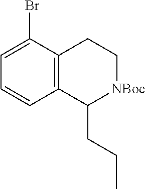 [SMILES: CCCC1c2cccc(Br)c2CCN1C(=O)OC(C)(C)C] | 298.2 [M + H − tBu]⁺/78% |

### General Procedure BR-THIQ 6

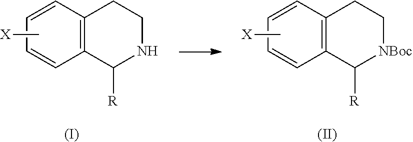

To a stirred solution of amine (I) (1 eq) in DCM was added Et3N (3 eq) followed by Boc-anhydride (1.5 eq) at 0° C. The reaction mixture was stirred at RT for 4 h before being quenched with ice-water and extracted with DCM. The combined organic layer was dried over sodium sulphate, filtered and concentrated in vacuo to afford Boo-protected compound (II).

### THIQ-2: tert-butyl 5-bromo-1-cyclopropyl-3,4-dihydro-1H-isoquinoline-2-carboxylate

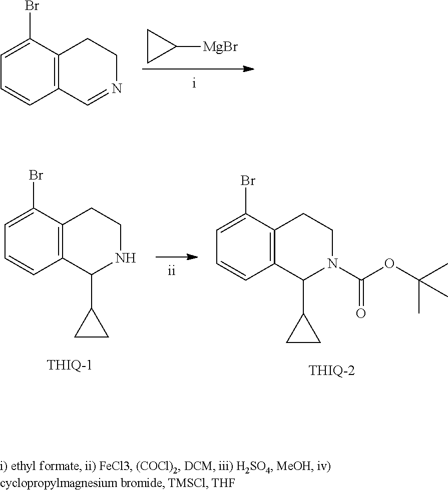

THIQ 1: To a stirred solution of 5-bromo-3,4-dihydroisoquinoline (5.3 g, 25.23 mmol, 1 eq) in THF (60 mL) was added trdmethylchlorosilane (1 M in THF, 5.04 mL, 5.04 mmol, 0.2 eq) at −20° C. The reaction was stirred for 30 min at −20° C. before cyclopropyl magnesium bromide (1 M in THF, 151.4 mL, 151.4 mmol, 6 eq) was added dropwise at −20° C. The reaction mixture was warmed slowly to RT then was heated to 65° C. overnight. The reaction was quenched with aqueous ammonium chloride solution 10° C. before being extracted with EtOAc (50 mL×3). The combined organic layers were washed with water, brine solution, dried over sodium sulfate and concentrated in vacuo to afford the desired product which was used in the next step without further purification. LCMS m/z=254.0 [M+H]⁺

THIQ-2: Following general procedure D. Obtained 4.7 g, 32.7% yield. LCMS m/z=296.2 [M+H-tBu]⁺

### THIQ-8: tert-butyl 5-bromo-1,1-dimethyl-3,4-dihydroisoquinoline-2(1H)-carboxylate

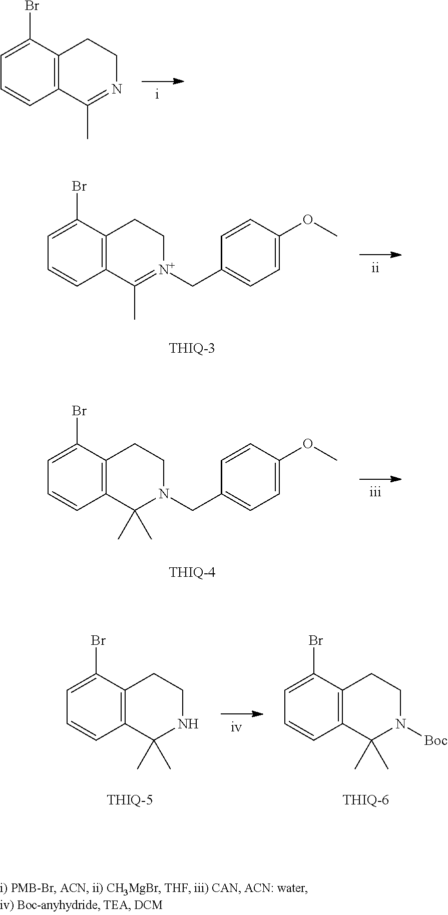

THIQ-3: To a stirred solution of 5-bromo-1-methyl-3,4-dihydroisoquinoline (14 g, 62.5 mmol) in MeCN (130 mL) and was added 1-(bromomethyl)-4-methoxybenzene (25.1 g, 125 mmol) at RT. The reaction mixture was then stirred at 80° C. for 16 h before being concentrated in vacuo. The resulting residue was dissolved in MeCN (50 mL) and was washed with MTBE (100 mL) to get afford 5-bromo-2-(4-methoxybenzyl)-1-methyl-3,4-dihydroisoquinolin-2-ium (25 g, 46.3 mmol, 74.2% yield). LCMS m/z=346.0 [M+H]⁺

THIQ-4: To a stirred solution of 5-bromo-2-(4-methoxybenzyl)-1-methyl-3,4-dihydroisoquinolin-2-ium (10 g, 18.82 mmol) in THF (250 mL) and was added methyl magnesium bromide (62.8 mL, 188 mmol) at 0° C.

The reaction mixture was then stirred at RT overnight before being quenched with aqueous NH₄Cl solution and extracted with EtOAc. The organic layer was dried over sodium sulphate and concentrated in vacuo. The resulting residue was purified by silica gel column chromatography (gradient=10% EtOAc in hexane). The appropriate fractions were concentrated in vacuo before being re-purified by reverse phase chromatography (gradient=0-100 0.1% ammonium acetate in water, in MeCN). The appropriate fractions were concentrated in vacuo to obtain 5-bromo-2-(4-methoxybenzyl)-1,1-dimethyl-1,2,3,4-tetrahydroisoquinoline (1.7 g, 4.72 mmol, 80% yield). LCMS m/z=362.2 [M+H]⁺.

THIQ-5: To a stirred solution of tert-butyl5-bromo-2-(4-methoxybenzyl)-1,1-dimethyl-1,2,3,4-tetrahydroisoquinoline (1.7 g, 4.72 mmol) in MeCN (15 mL) and water (2.5 mL) was added CAN (7.76 g, 14.16 mmol, 3 eq) at 0° C. The reaction mixture was stirred for 16 h at RT before being concentrated in vacuo. The resulting residue was purified by reverse phase column chromatography (0 gradient=0-100 0.1% ammonium acetate in water, in MeCN). The appropriate fractions were concentrated in vacuo to obtain 5-bromo-1,1-dimethyl-1,2,3,4-tetrahydroisoquinoline (0.700 g, 2.77 mmol, 85% yield). LCMS m/z=240.2 [M+H]⁺

THIQ-6: Following general procedure BR-THIQ 6. Obtained 1.5 g, 88% yield. LCMS m/z=286.2 [M+H−tBu]⁺

### THIQ-11: tert-butyl 5-bromo-1-isopropyl-3,4-dihydroisoquinoline-2(1H)-carboxylate

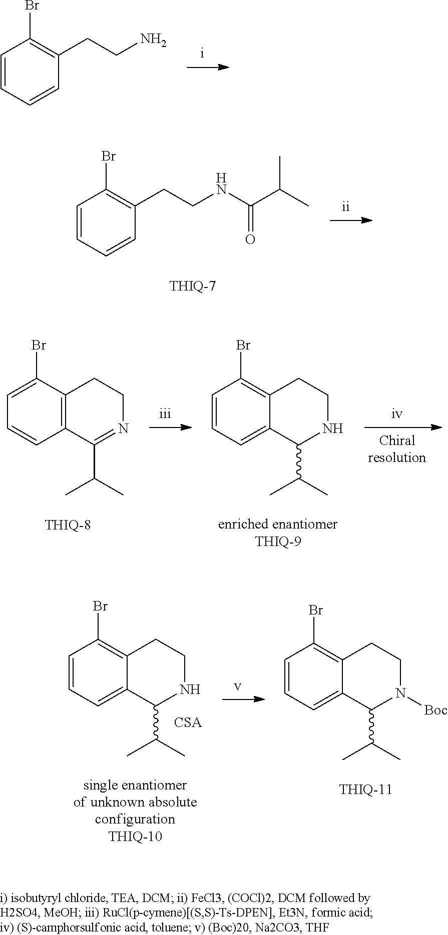

THIQ-7: To a stirred solution of 2-(2-bromophenyl) ethan-1-amine (500 g, 2.499 mol) in DCM (5 L) was added Et3N (522 ml, 3.75 mol) and the reaction was stirred for 30 min at 0° C. Isobutyryl chloride (262 mL, 2.499 mol) was added drop wise at 0-5° C. over a period of 30 min. The reaction mixture was allowed to warm to 25° C. and stirred for 2 h before being diluted with water (5 L) and the organic layer was separated. The aqueous layer was extracted with DCM (5 L) and the combined organic layers were washed with water and dried over anhydrous Na₂SO₄, filtered and concentrated in vacuo. The resulting residue was triturated with n-hexane (2.5 L) to give N-(2-bromophenethyl) isobutyramide (600 g, 88% yield). LCMS m/z=272.1 [M+H]⁺

THIQ-8: To a stirred solution of N-(2-bromophenethyl) isobutyramide (250 g, 925 mmol) in DCM (2.5 L) was added oxalyl chloride (198 mL, 2.313 mol) drop wise at 0-5° C. and the reaction mixture was stirred for 1 h. The mixture was warmed to RT and stirred for 2 h. The reaction mixture was cooled to 0-5° C. and iron(III) chloride (150 g, 925 mmol) was added portionwise. The reaction mixture was stirred at RT for 16 h before being diluted with DCM (2.5 L), filtered through celite, and washed with excess DCM (1.5 L×2). The combined organic layers were concentrated in vacuo to remove excess oxalyl chloride. The residue was dissolved in water (4 L) and extracted with DCM (2×4 L). The combined organic layers were dried over anhydrous Na₂SO₄ and concentrated in vacuo. The resulting residue was triturated with methanol (1 L), then stirred for 30 minutes and filtered to get the required Intermediate (252 g) as off-white solid. The intermediate was dissolved in H₂SO₄:MeOH (1:10, 2.2 L) at 0-10° C. and stirred at 80° C. for 18 h before being concentrated in vacuo to remove excess MeOH. The resulting residue was dissolved in water (2.5 L) and the pH was adjusted to 8-9 using 25% aq. ammonia (1 L). The product was extracted with DCM (3×2 L) and the combined organic layers were dried over anhydrous Na₂SO₄ and concentrated in vacuo. The crude compound was used in the next step without further purification. LCMS m/z=252.0 [M+H]⁺

THIQ-9: A stirred solution of RuCl(p-cymene) [(S,S)-Ts-DPEN](10.09 g, 15.86 mmol) in MeOH (2 L), was purged with argon for 15 min. 5-bromo-1-isopropyl-3,4-dihydroisoquinoline (200 g, 793 mmol) was added, followed by formic acid (180 mL, 4.759 mol) under argon. The mixture was cooled to −5° C. and Et3N (332 mL, 2.379 mol) was added dropwise while maintaining the temperature at −5 to 0° C. over a period of 1 h. The mixture was slowly warmed to 25° C. and stirred for 24 h. After completion of reaction, the reaction mixture was diluted with water (2 L) and basified with aqueous Na₂CO₃ (pH=8-9) at 0-5° C. The mixture was extracted with DCM (2×2 L) and the combined organic layers were washed with water (2 L×3), brine (2 L), dried over Na₂SO₄and concentrated in vacuo to get crude product which was used for next step without any further purification. LCMS m/z=254.1 [M+H]⁺

THIQ-10: To a stirred solution of 5-bromo-1-isopropyl-1,2,3,4-tetrahydroisoquinoline (200 g, 787 mmol) in toluene (10 L) was added (S)-camphor sulfonic acid (187 g, 787 mmol) at RT. The mixture was stirred at reflux for 1 h before being cooled to 45° C. gradually over 5-6 h. The reaction mixture was stirred at 45° C. for 1 h and the resulting solid was filtered, washed with toluene (1 L), and dried under high vacuum to get the desired product (252 g, 67.7% yield) as an off-white solid. LCMS m/z=254.2 [M+H]⁺

THIQ-11: To a stirred solution of 5-bromo-1-isopropyl-1,2,3,4-tetrahydroisoquinoline (S)-camphor sulfonic acid (250 g, 529 mmol) in THF (2.5 L) and water (2.5 L) was added Na₂CO₃ (123 g, 1.164 mol) portion wise at RT. Boc-anhydride (127 g, 582 mmol) was added dropwise and the reaction was stirred for 1 h before being diluted with water (2.5 L) and extracted with EtOAc (2×3 L). The combined organic layers were washed with water (1.25 L), brine (1.25 L), dried over Na₂SO₄, and concentrated in vacuo. The resulting residue was purified by flash chromatography (gradient=0-5% EtOAc in n-hexane) to afford the desired enantiomer of tert-butyl 5-bromo-1-isopropyl-3,4-dihydroisoquinoline-2(1H)-carboxylate (162 g, 86% yield) as a white solid. LCMS m/z=254.2 [M+H-Boc]⁺

### Intermediate W-20: 1-methyl-1,2,3,4-tetrahydroisoquinoline-5-carbaldehyde

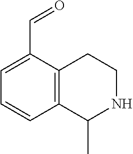

Prepared following general procedure 2 from tert-butyl 5-formyl-1-methyl-1,2,3,4-tetrahydroisoquinoline-2-carboxylate (prepared as described in WO2017/68412). Obtained 190 mg, 78% yield. LCMS m/z=175.3 [M+H]⁺.

### Intermediate W-21: 1-methyl-2-(2,2,2-trifluoroacetyl)-1,2,3,4-tetrahydroisoquinoline-5-carbaldehyde

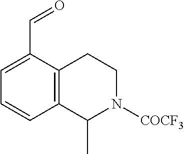

To a stirred solution of 1-methyl-1,2,3,4-tetrahydroisoquinoline-5-carbaldehyde hydrochloride (100 mg, 0.47 mmol) and Et3N (0.3 mL, 2.36 mmol) in DCM (3 mL) was added trifluoroacetic anhydride (0.1 mL, 0.71 mmol) at 0° C. The reaction mixture was stirred at RT for 16 h then was quenched with water and extracted with DCM. The organic layer was dried over anhydrous sodium sulphate and concentrated in vacuo to afford 1-methyl-2-(2,2,2-trifluoroacetyl)-1,2,3,4-tetrahydroisoquinoline-5-carbaldehyde (120 mg, 0.41 mmol, 85% yield). LCMS m/z=272.3 [M+H]⁺.

### Intermediate W-22: tert-butyl 5-formyl-1-methyl-3,4-dihydroisoquinoline-2(1H)-carboxylate

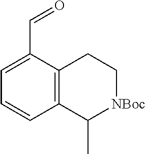 [SMILES: CC1c2cccc(C=O)c2CCN1C(=O)OC(C)(C)C]

Synthesised as described in WO2022/129925

### Intermediates W-23 to W-27

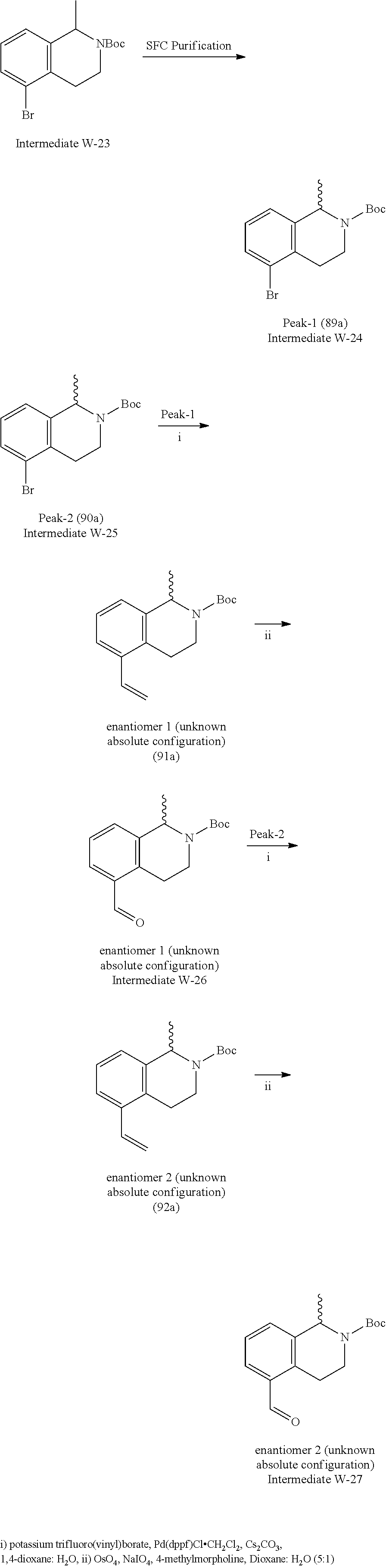

Intermediate W-23: Prepared as described in WO20221129925

Racemic tert-butyl 5-bromo-1-methyl-3,4-dihydroisoquinoline-2(1H)-carboxylate (50 g) was purified by chiral SFC:

| | |
|---|---|
| Column | WHELK RR-(250*30 mm), 5 μm |
| Mobile Phase | CO₂: 0.5% isopropylamine in MeOH:MeCN [90:10] |
| Flow rate | 5 mL/min |
| Back pressure | 100 bar |
| Wavelength | 220 nm |
| Cycle time | 6 min |

Isolated Peak-1 (89a): 22 g, 44% yield. LCMS m/z=226.3 [M-Boc]⁺ and Peak-2 (90a): 22 g, 43% yield. LCMS m/z=226.3 [M-Boc]⁺

### Enantiomer 1 of tert-butyl 1-methyl-5-vinyl-3,4-dihydroisoquinoline-2(1H)-carboxylate (91a)

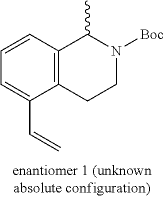 [SMILES: C=Cc1cccc2c1CCN(C(=O)OC(C)(C)C)C2C]

Prepared following general procedure 30. Obtained 785 mg, 92% yield. LCMS m/z=174.2 [M-Boc]⁺.

### Enantiomer 1 of tert-butyl 5formyl-1-methyl-3,4-dihydroisoquinoline-2(1H)-carboxylate (Intermediate W-26)

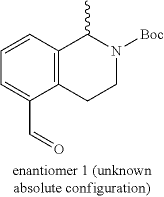 [SMILES: CC1c2cccc(C=O)c2CCN1C(=O)OC(C)(C)C]

Prepared following general procedure 31. Obtained 510 mg, 65% yield. LCMS m/z=176.4 [M-Boc]⁺.

### Enantiomer 2 of tert-butyl 1-methyl-6-vinyl-3,4-dihydroisoquinoline-2(1H)-carboxylate (92a)

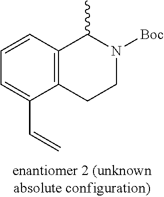 [SMILES: C=Cc1cccc2c1CCN(C(=O)OC(C)(C)C)C2C]

Prepared following general procedure 30. Obtained 3.89 g, 92% yield. LCMS m/z=174.4 [M−56]⁺.

### Enantiomer 2 of tert-butyl 5-formyl-1-methyl-3,4-dihydroisoquinoline-2(1H)-carboxylate (Intermediate W-27)

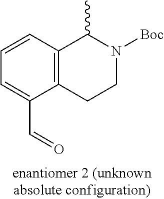 [SMILES: CC1c2cccc(C=O)c2CCN1C(=O)OC(C)(C)C]

Prepared following general procedure 31. Obtained 600 mg, 70% yield. LCMS m/z=176.4 [M-Boc]r.

### Intermediate W-28: 1-(5-(bromomethyl)-1-methyl-3,4-dihydroisoquinolin-2(1H)-yl)-2,2,2-trifluoroethan-1-one2,2,2-trifluoro-1-(1-methyl-5-vinyl-3,4-dihydroisoquinolin-2(1H)-yl)ethan-1-one

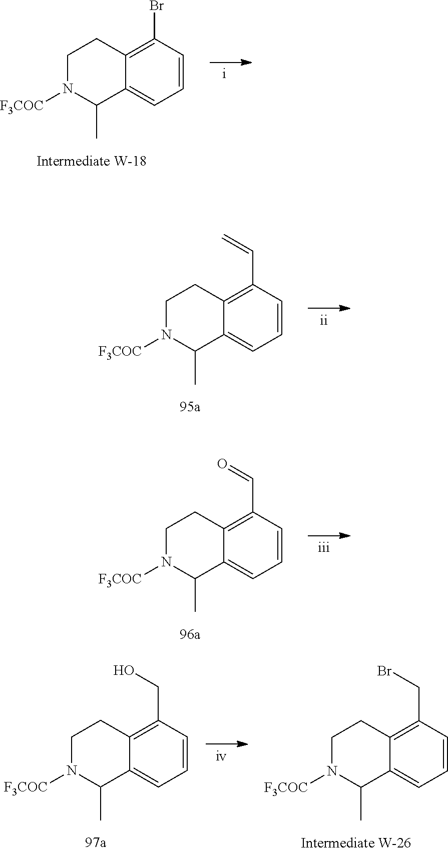

95a: Prepared following general procedure 28. Obtained 900 mg, 88% yield. LCMS m/z=170.3 [M-Boc]⁺

96a: Prepared following general procedure 29. Obtained 600 mg, 62% yield. Used without further purification.

97a: To a stirred solution of 1-methyl-2-(2,2,2-trifluoroacetyl)-1,2,3,4-tetrahydroisoquinoline-5-carbaldehyde (600 mg, 2.21 mmol, 1 eq) in THF (10 mL) at 0° C. was added NaBH₄ (126 mg, 3.32 mmol, 1.5 eq) and the reaction was stirred at RT for 2 h. The reaction was quenched with ice-water and extracted with EtOAc. The organic layer was concentrated in vacuo to afford crude 2,2,2-trifluoro-1-(5-(hydroxymethyl)-1-methyl-3,4-dihydroisoquinolin-2(1H)-yl)ethan-1-one (600 mg, 2.196 mmol, 99% yield). Used without further purification.

Intermediate W-28: To a stirred solution of 2,2,2-trifluoro-1-(5-(hydroxymethyl)-1-methyl-3,4-dihydroisoquinolin-2(1H)-yl)ethan-1-one (600 mg, 2.196 mmol, 1 eq) in DCM (10 mL) was added CBr₄ (1.092 g, 3.29 mmol, 1.5 eq) and triphenylphosphine (864 mg, 3.29 mmol, 1.5 eq) at 0° C. The reaction mixture was stirred at RT for 2 h before being quenched with Ice cold water and extracted with DCM. The organic layer was concentrated in vacuo and the resulting residue was purified by silica gel column chromatography (gradient=0-10% EtOAc in hexane). The appropriate fractions were concentrated in vacuo to afford 1-(5-(bromomethyl)-1-methyl-3,4-dihydroisoquinolin-2(1H)-yl)-2,2,2-trifluoroethan-1-one (430 mg, 1.279 mmol, 58.3% yield).

Table 1a: The following examples were prepared by general procedure 21 from Intermediates W-12 to W-16, W-19, and W-23 to W-25 using the relevant commercially available mono protected diamine.

TABLE 1a

| | | LCMS |
|---|---|---|
| | | m/z [M + H]⁺/ |
| Name | Product Structure | Yield |
| tert-butyl 2-(1-methyl-2-(2,2,2- trifluoroacetyl)-1,2,3,4- tetrahydroisoquinolin-5-yl)-2,6- diazaspiro[3.4]octane-6-carboxylate | 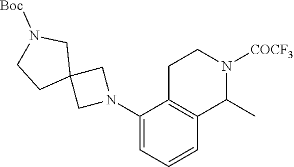 | 454.2/81% |
| tert-butyl 9-(1-methyl-2-(2,2,2- trifluoroacetyl)-1,2,3,4- tetrahydroisoquinolin-5-yl)-3,9- diazaspiro[5.5]undecane-3-carboxylate | 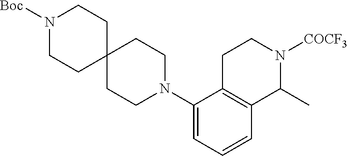 | 496.2/51% |
| tert-butyl 7-(1-methyl-2-(2,2,2- trifluoroacetyl)-1,2,3,4- tetrahydroisoquinolin-5-yl)-2,7- diazaspiro[4.4]nonane-2-carboxylate | 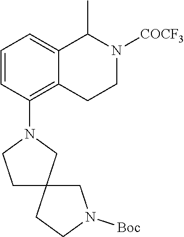 | 468.2/95% |
| tert-butyl 5-(4- ((benzyloxy)carbonyl)piperazin-1-yl)-1- methyl-3,4-dihydroisoquinoline-2(1H)- carboxylate | 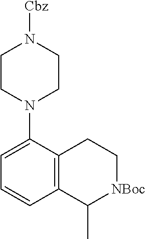 [SMILES: CC1c2cccc(N3CCN(C(=O)OCc4ccccc4)CC3)c2CCN1C(=O)OC(C)(C)C] | 466.5/75% |
| tert-butyl methyl(1-(1-methyl-2-(2,2,2- trifluoroacetyl)-1,2,3,4- tetrahydroisoquinolin-5-yl)piperidin-4- yl)carbamate | 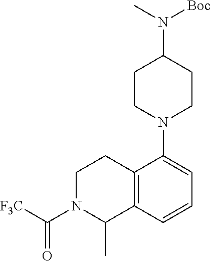 [SMILES: CC1c2cccc(N3CCC(N(C)C(=O)OC(C)(C)C)CC3)c2CCN1C(=O)C(F)(F)F] | 456.5/56% |
| tert-butyl 2-(1-methyl-2-(2,2,2- trifluoroacetyl)-1,2,3,4- tetrahydroisoquinolin-5-yl)-2,8- diazaspiro[4.5]decane-8-carboxylate | 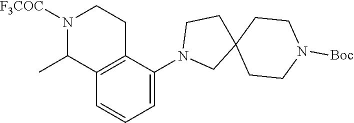 | 426.2/50% |
| tert-butyl 2-(1-methyl-2-(2,2,2- trifluoroacetyl)-1,2,3,4- tetrahydroisoquinolin-5-yl)-2,7- diazaspiro[3.5]nonane-7-carboxylate | 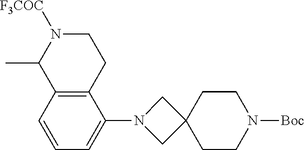 | 368.2 [M − Boc]⁺/69% |
| tert-butyl 3-(methyl(1-methyl-2-(2,2,2- trifluoroacetyl)-1,2,3,4- tetrahydroisoquinolin-7- yl)amino) azetidine-1-carboxylate | 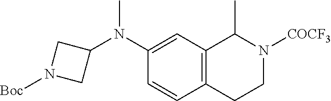 | 372.2 [M − tBu]⁺/67% |
| benzyl 7-(((1-(tert- butoxycarbonyl)piperidin-4- yl)methyl)(methyl)amino)-1-methyl-3,4- dihydroisoquinoline-2(1H)-carboxylate | 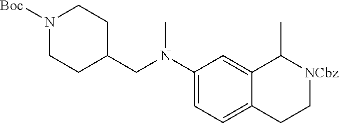 [SMILES: CC1c2cc(N(C)CC3CCN(C(=O)OC(C)(C)C)CC3)ccc2CCN1C(=O)OCc1ccccc1] | 408.1 [M − Boc]⁺/ |
| benzyl 2-(2-(tert-butoxycarbonyl)-1- methyl-1,2,3,4-tetrahydroisoquinolin-5- yl)-2,8-diazaspiro[4.5]decane-8- carboxylate | 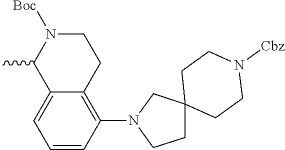 [SMILES: CC1c2cccc(N3CCC4(CCN(C(=O)OCc5ccccc5)CC4)C3)c2CCN1C(=O)OC(C)(C)C] | 520.4/31% |
| | single enantiomer of unknown absolute | |
| | configuration using peak 2 of tert-butyl 5-bromo-1- | |
| | methyl-3,4-dihydroisoquinoline-2(1H)-carboxylate | |
| tert-butyl 7-(1-methyl-2-(2,2,2- trifluoroacetyl)-1,2,3,4- tetrahydroisoquinolin-7-yl)-2,7- diazaspiro[4.4]nonane-2-carboxylate | 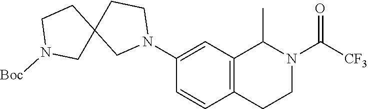 [SMILES: CC1c2cc(N3CCC4(CCN(C(=O)OC(C)(C)C)C4)C3)ccc2CCN1C(=O)C(F)(F)F] | 412.2 [M − 56 + H]⁺/95% |
| Single enantiomer of tert-butyl 8-(1- methyl-2-(2,2,2-trifluoroacetyl)-1,2,3,4- tetrahydroisoquinolin-7-yl)-2,8- diazaspiro[4.5]decane-2-carboxylate | 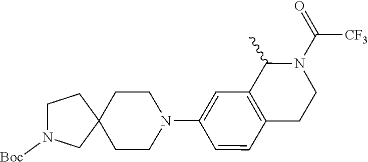 [SMILES: CC1c2cc(N3CCC4(CCN(C(=O)OC(C)(C)C)C4)CC3)ccc2CCN1C(=O)C(F)(F)F] | 482.2/77% |
| | enantiomer 1 | |
| | single enantiomer of unknown | |
| | absolute configuration | |
| Single enantiomer tert-butyl 5-(7- ((benzyloxy)carbonyl)-2,7- diazaspiro[3.5]nonan-2-yl)-1-methyl- 3,4-dihydroisoquinoline-2(1H)- carboxylate | 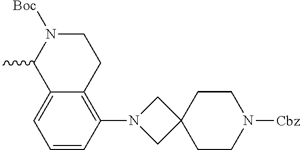 [SMILES: CC1c2cccc(N3CC4(CCN(C(=O)OCc5ccccc5)CC4)C3)c2CCN1C(=O)OC(C)(C)C] | 506.3/55% |
| | single enantiomer of unknown absolute | |
| | configuration using peak 2 of tert-butyl 5-bromo-1- | |
| | methyl-3,4-dihydroisoquinoline-2(1H)-carboxylate | |
| | and starting material prepared according to | |
| | WO2022/198112 | |
| Single enantiomer of tert-butyl 8-(1- methyl-2-(2,2,2-trifluoroacetyl)-1,2,3,4- tetrahydroisoquinolin-7-yl)-2,8- diazaspiro[4.5]decane-2-carboxylate | 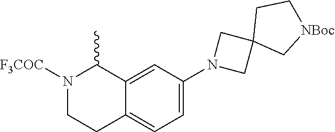 | 454.2/80% |
| | enantiomer 1 | |
| | single enantiomer of unknown absolute | |
| | configuration using peak 1 of tert-butyl 7-bromo-1- | |
| | methyl-3,4-dihydroisoquinoline-2(1H)-carboxylate | |
| Single enantiomer of tert-butyl 4- (methyl(1-methyl-2-(2,2,2- trifluoroacetyl)-1,2,3,4- tetrahydroisoquinolin-7- yl)amino)piperidine-1-carboxylate | 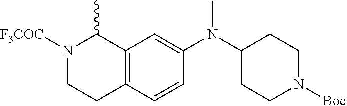 | 456.2/55% |
| | enantiomer 1 | |
| | single enantiomer of unknown absolute | |
| | configuration using peak 1 of tert-butyl 7-bromo-1- | |
| | methyl-3,4-dihydroisoquinoline-2(1H)-carboxylate | |
| tert-butyl 2-(1-methyl-2-(2,2,2- trifluoroacetyl)-1,2,3,4- tetrahydroisoquinolin-7-yl)-2,8- diazaspiro[4.5]decane-8-carboxylate | 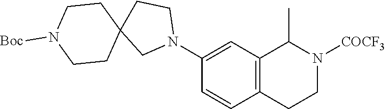 | 482.2/95% |
| Enantiomer 1 of tert-butyl 1-methyl-7-(4- (2,2,2-trifluoroacetyl)-1,4-diazepan-1- yl)-3,4-dihydroisoquinoline-2(1H)- carboxylate | 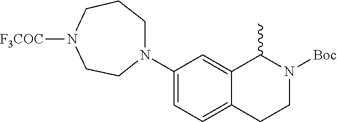 | 442.2/75% |
| | single enantiomer of unknown absolute | |
| | configuration using peak 1 of tert-butyl 7-bromo-1- | |
| | methyl-3,4-dihydroisoquinoline-2(1H)-carboxylate | |
| Enantiomer 2 of tert-butyl 1-methyl-7-(4- (2,2,2-trifluoroacetyl)-1,4-diazepan-1- yl)-3,4-dihydroisoquinoline-2(1H)- carboxylate | 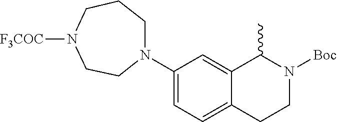 | 442.2/79% |
| | single enantiomer of unknown absolute | |
| | configuration using peak 2 of fert-butyl 7-bromo-1- | |
| | methyl-3,4-dihydroisoquinoline-2(1H)-carboxylate | |

Table 2a: The following examples were prepared by general procedure 14 using the relevant aldehyde (synthesized according to WO2021178920) and relevant commercially available mono protected diamine.

TABLE 2a

| | | | LCMS m/z |
|---|---|---|---|
| | | | [M + H]⁺/ |
| Name | Amine (Y) | Product Structure | Yield |
| tert-butyl 4-(2,6-dimethoxy-4-(2- methyl-1-oxo-1,2-dihydro-2,7- naphthyridin-4-yl)benzyl)-1,4- diazepane-1-carboxylate | 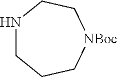 [SMILES: CC(C)(C)OC(=O)N1CCCNCC1] | 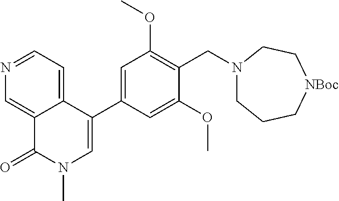 [SMILES: COc1cc(-c2cn(C)c(=O)c3cnccc23)cc(OC)c1CN1CCCN(C(=O)OC(C)(C)C)CC1] | 509.2/88% |
| tert-butyl (2-((2,6-dimethoxy-4-(2- methyl-1-oxo-1,2-dihydro-2,7- naphthyridin-4- yl)benzyl)(methyl)amino)ethyl) (methyl)carbamate | 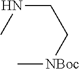 | 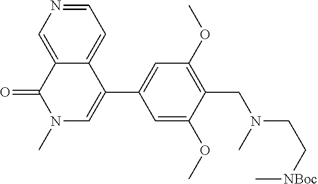 [SMILES: COc1cc(-c2cn(C)c(=O)c3cnccc23)cc(OC)c1CN(C)CCN(C)C(=O)OC(C)(C)C] | 497.2/91% |
| tert-butyl 5-(2,6-dimethoxy-4-(2- methyl-1-oxo-1,2-dihydro-2,7- naphthyridin-4-yl)benzyl)-2,5- diazabicyclo[2.2.1]heptane-2- carboxylate | 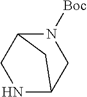 [SMILES: CC(C)(C)OC(=O)N1CC2CC1CN2] | 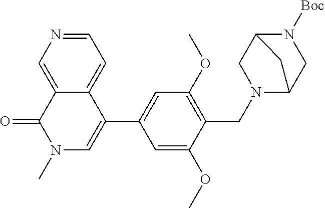 [SMILES: COc1cc(-c2cn(C)c(=O)c3cnccc23)cc(OC)c1CN1CC2CC1CN2C(=O)OC(C)(C)C] | 507.0/75% |
| tert-butyl 6-(2,6-dimethoxy-4-(2- methyl-1-oxo-1,2-dihydro-2,7- naphthyridin-4-yl)benzyl)-3,6- diazabicyclo[3.1.1]heptane-3- carboxylate | 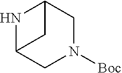 [SMILES: CC(C)(C)OC(=O)N1CC2CC(C1)N2] | 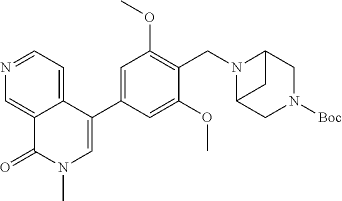 [SMILES: COc1cc(-c2cn(C)c(=O)c3cnccc23)cc(OC)c1CN1C2CC1CN(C(=O)OC(C)(C)C)C2] | 507.0/41% |
| tert-butyl 3-(2,6-dimethoxy-4-(2- methyl-1-oxo-1,2-dihydro-2,7- naphthyridin-4-yl)benzyl)-3,6- diazabicyclo[3.1.1]heptane-6- carboxylate | 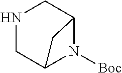 [SMILES: CC(C)(C)OC(=O)N1C2CNCC1C2] | 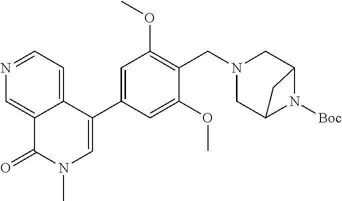 [SMILES: COc1cc(-c2cn(C)c(=O)c3cnccc23)cc(OC)c1CN1CC2CC(C1)N2C(=O)OC(C)(C)C] | 507.0/77% |
| tert-butyl 3-(2,6-dimethoxy-4-(2- methyl-1-oxo-1,2-dihydro-2,7- naphthyridin-4-yl)benzyl)-3,8- diazabicyclo[3.2.1]octane-8- carboxylate | 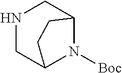 [SMILES: CC(C)(C)OC(=O)N1C2CCC1CNC2] | 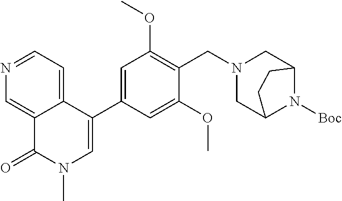 [SMILES: COc1cc(-c2cn(C)c(=O)c3cnccc23)cc(OC)c1CN1CC2CCC(C1)N2C(=O)OC(C)(C)C] | 521.1/90% |
| tert-butyl 4-(2,6-dimethoxy-4- (1,4,5-trimethyl-6-oxo-1,6- dihydropyridin-3- yl)benzyl)piperazine- 1-carboxylate | 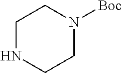 [SMILES: CC(C)(C)OC(=O)N1CCNCC1] | 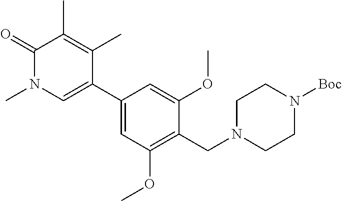 [SMILES: COc1cc(-c2cn(C)c(=O)c(C)c2C)cc(OC)c1CN1CCN(C(=O)OC(C)(C)C)CC1] | 472.2/96% |
| tert-butyl 5-(2,6-dimethoxy-4- (1,4,5-trimethyl-6-oxo-1,6- dihydropyridin-3- yl)benzyl)-2,5- diazabicyclo[2.2.1]heptane-2- carboxylate | 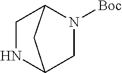 [SMILES: CC(C)(C)OC(=O)N1CC2CC1CN2] | 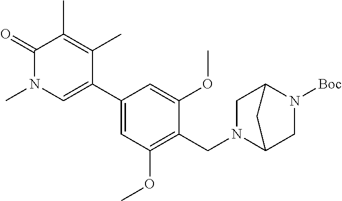 [SMILES: COc1cc(-c2cn(C)c(=O)c(C)c2C)cc(OC)c1CN1CC2CC1CN2C(=O)OC(C)(C)C] | 484.3/95% |
| tert-butyl 3-(2,6-dimethoxy-4- (1,4,5-trimethyl-6-oxo-1,6- dihydropyridin-3- yl)benzyl)-3,6- diazabicyclo[3.1.1]heptane-6- carboxylate | 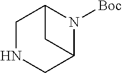 [SMILES: CC(C)(C)OC(=O)N1C2CNCC1C2] | 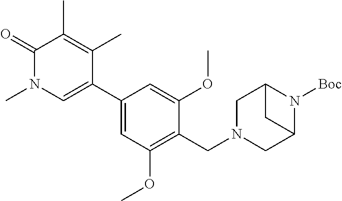 [SMILES: COc1cc(-c2cn(C)c(=O)c(C)c2C)cc(OC)c1CN1CC2CC(C1)N2C(=O)OC(C)(C)C] | 484.5/95% |
| tert-butyl 6-(2,6-dimethoxy-4- (1,4,5-trimethyl-6-oxo-1,6- dihydropyridin-3- yl)benzyl)-3,6- diazabicyclo[3.1.1]heptane-3- carboxylate | 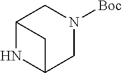 [SMILES: CC(C)(C)OC(=O)N1CC2CC(C1)N2] | 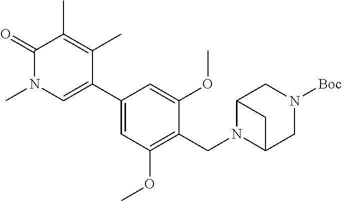 [SMILES: COc1cc(-c2cn(C)c(=O)c(C)c2C)cc(OC)c1CN1C2CC1CN(C(=O)OC(C)(C)C)C2] | 484.3/92% |
| tert-butyl 3-(2,6-dimethoxy-4- (1,4,5-trimethyl-6-oxo-1,6- dihydropyridin-3- yl)benzyl)-3,8- diazabicyclo[3.2.1]octane-8- carboxylate | 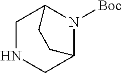 [SMILES: CC(C)(C)OC(=O)N1C2CCC1CNC2] | 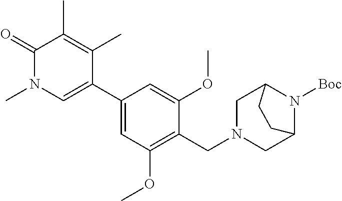 [SMILES: COc1cc(-c2cn(C)c(=O)c(C)c2C)cc(OC)c1CN1CC2CCC(C1)N2C(=O)OC(C)(C)C] | 498.4/91% |
| tert-butyl 4-(2,6-dimethoxy-4- (1,4,5-trimethyl-6-oxo-1,6- dihydropyridin-3- yl)benzyl)-1,4-diazepane-1- carboxylate | 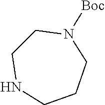 [SMILES: CC(C)(C)OC(=O)N1CCCNCC1] | 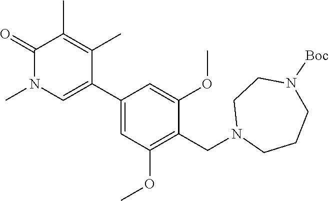 [SMILES: COc1cc(-c2cn(C)c(=O)c(C)c2C)cc(OC)c1CN1CCCN(C(=O)OC(C)(C)C)CC1] | 486.4/76% |
| tert-butyl 4-((2,6-dimethoxy-4- (1,4,5-trimethyl-6-oxo-1,6- dihydropyridin-3- yl)benzyl)(methyl)amino) piperidine-1-carboxylate | 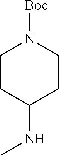 | 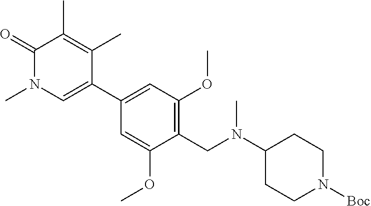 [SMILES: COc1cc(-c2cn(C)c(=O)c(C)c2C)cc(OC)c1CN(C)C1CCN(C(=O)OC(C)(C)C)CC1] | 500.2/97% |
| tert-butyl 7-(2,6-dimethoxy-4- (1,4,5-trimethyl-6-oxo-1,6- dihydropyridin-3- yl)benzyl)-4,7-diazaspiro[2.5] octane-4-carboxylate | 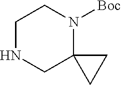 [SMILES: CC(C)(C)OC(=O)N1CCNCC12CC2] | 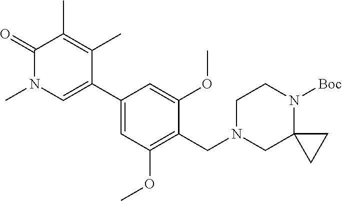 [SMILES: COc1cc(-c2cn(C)c(=O)c(C)c2C)cc(OC)c1CN1CCN(C(=O)OC(C)(C)C)C2(CC2)C1] | 498.3/95% |
| tert-butyl 4-(2,6-dimethoxy-4- (1,4,5-trimethyl-6-oxo-1,6- dihydropyridin-3- yl)benzyl)-4,7-diazaspiro[2.5] octane-7-carboxylate | 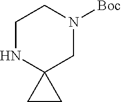 [SMILES: CC(C)(C)OC(=O)N1CCNC2(CC2)C1] | 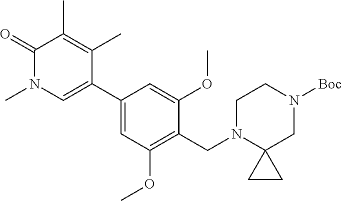 [SMILES: COc1cc(-c2cn(C)c(=O)c(C)c2C)cc(OC)c1CN1CCN(C(=O)OC(C)(C)C)CC12CC2] | 498.4/94% |
| tert-butyl 9-(2,6-dimethoxy-4- (1,4,5-trimethyl-6-oxo-1,6- dihydropyridin-3- yl)benzyl)-3,9- diazaspiro[5.5]undecane-3- carboxylate | 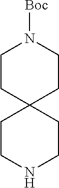 [SMILES: CC(C)(C)OC(=O)N1CCC2(CCNCC2)CC1] | 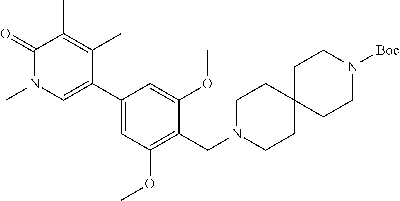 [SMILES: COc1cc(-c2cn(C)c(=O)c(C)c2C)cc(OC)c1CN1CCC2(CC1)CCN(C(=O)OC(C)(C)C)CC2] | 540.5/85% |
| tert-butyl (2,6-dimethoxy-4- (1,4,5-trimethyl-6-oxo-1,6- dihydropyridin-3- yl)benzyl)(methyl)carbamate | 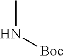 | 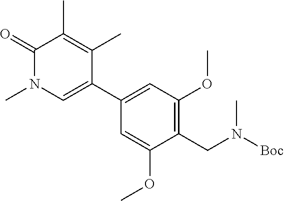 [SMILES: COc1cc(-c2cn(C)c(=O)c(C)c2C)cc(OC)c1CN(C)C(=O)OC(C)(C)C] | 417.4/76% |
| tert-butyl (2R,5S)-4-(2,6- dimethoxy-4-(1,4,5-trimethyl- 6-oxo-1,6-dihydropyridin-3- yl)benzyl)-2,5- dimethylpiperazine-1- carboxylate | 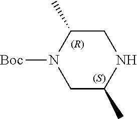 | 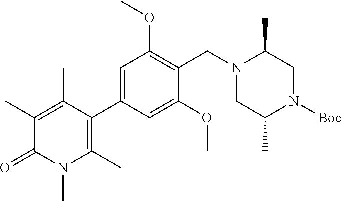 [SMILES: COc1cc(-c2c(C)c(C)c(=O)n(C)c2C)cc(OC)c1CN1C[C@@H](C)N(C(=O)OC(C)(C)C)C[C@@H]1C] | 500.3/98% |
| tert-butyl 4-(2,6-dimethoxy-4- (1,4,5-trimethyl-6-oxo-1,6- dihydropyridin-3- yl)benzyl)-2,2- dimethylpiperazine-1- carboxylate | 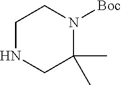 | 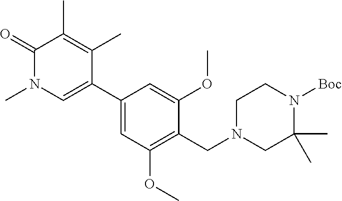 | 500.4/81% |
| tert-butyl (2S,6S)-4-(2,6- dimethoxy- 4-(1,4,5-trimethyl-6-oxo-1,6- dihydropyridin-3-yl)benzyl)-2,6- dimethylpiperazine-1-carboxylate | 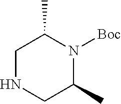 | 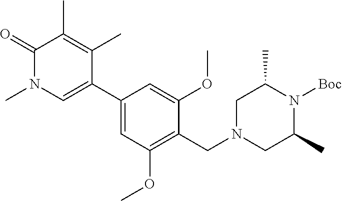 [SMILES: COc1cc(-c2cn(C)c(=O)c(C)c2C)cc(OC)c1CN1C[C@H](C)N(C(=O)OC(C)(C)C)[C@@H](C)C1] | 500.3/98% |
| tert-butyl 5-(2,6-dimethoxy-4- (1,4,5-trimethyl-6-oxo-1,6- dihydropyridin-3- yl)benzyl)-2,5- diazabicyclo[2.2.2]octane-2- carboxylate | 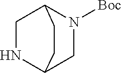 [SMILES: CC(C)(C)OC(=O)N1CC2CCC1CN2] | 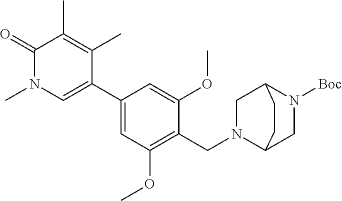 [SMILES: COc1cc(-c2cn(C)c(=O)c(C)c2C)cc(OC)c1CN1CC2CCC1CN2C(=O)OC(C)(C)C] | 498.2/83% |
| tert-butyl (R)-4-(2,6-dimethoxy- 4- (1,4,5-trimethyl-6-oxo-1,6- dihydropyridin-3-yl)benzyl)-2- methylpiperazine-1-carboxylate | 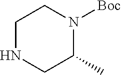 | 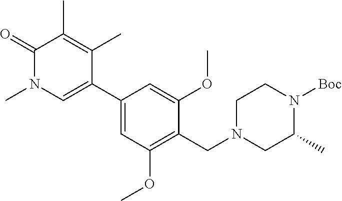 [SMILES: COc1cc(-c2cn(C)c(=O)c(C)c2C)cc(OC)c1CN1CCN(C(=O)OC(C)(C)C)[C@H](C)C1] | 486.2/98% |
| tert-butyl (S)-4-(2,6-dimethoxy- 4- (1,4,5-trimethyl-6-oxo-1,6- dihydropyridin-3-yl)benzyl)-2- methylpiperazine-1-carboxylate | 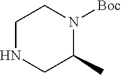 | 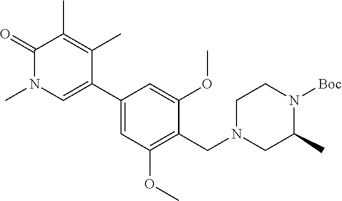 [SMILES: COc1cc(-c2cn(C)c(=O)c(C)c2C)cc(OC)c1CN1CCN(C(=O)OC(C)(C)C)[C@@H](C)C1] | 486.3/98% |
| tert-butyl (3S,5R)-4-(2,6- dimethoxy-4-(1,4,5-trimethyl- 6-oxo-1,6-dihydropyridin-3- yl)benzyl)-3,5- dimethylpiperazine-1- carboxylate | 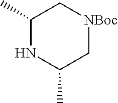 | 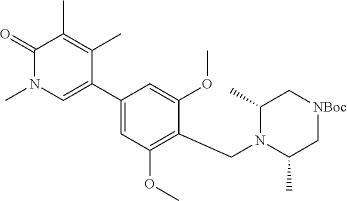 [SMILES: COc1cc(-c2cn(C)c(=O)c(C)c2C)cc(OC)c1CN1[C@H](C)CN(C(=O)OC(C)(C)C)C[C@@H]1C] | 500.4/78% |
| tert-butyl (2R,5R)-4-(2,6- dimethoxy-4-(1,4,5-trimethyl- 6-oxo-1,6-dihydropyridin-3- yl)benzyl)-2,5- dimethylpiperazine-1- carboxylate | 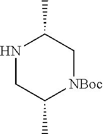 | 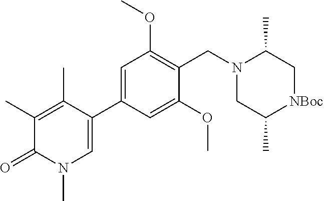 [SMILES: COc1cc(-c2cn(C)c(=O)c(C)c2C)cc(OC)c1CN1C[C@@H](C)N(C(=O)OC(C)(C)C)C[C@H]1C] | 500.4/83% |
| tert-butyl (R)-4-(2,6-dimethoxy- 4- (1,4,5-trimethyl-6-oxo-1,6- dihydropyridin-3-yl)benzyl)-2- ethylpiperazine-1-carboxylate | 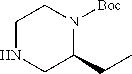 | 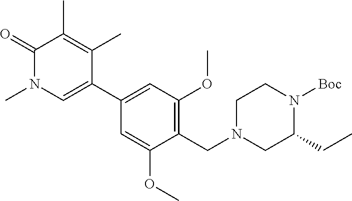 [SMILES: CC[C@@H]1CN(Cc2c(OC)cc(-c3cn(C)c(=O)c(C)c3C)cc2OC)CCN1C(=O)OC(C)(C)C] | 500.6/87% |
| tert-butyl (S)-4-(2,6-dimethoxy- 4- (1,4,5-trimethyl-6-oxo-1,6- dihydropyridin-3-yl)benzyl)-3- ethylpiperazine-1-carboxylate | 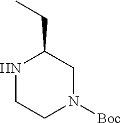 | 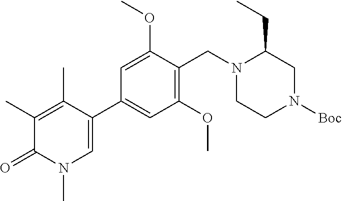 [SMILES: CC[C@H]1CN(C(=O)OC(C)(C)C)CCN1Cc1c(OC)cc(-c2cn(C)c(=O)c(C)c2C)cc1OC] | 500.4/94% |
| tert-butyl 5-(2,6-dimethoxy-4- (1,4,5-trimethyl-6-oxo-1,6- dihydropyridin-3- yl)benzyl)-2,5- diazabicyclo[4.1.0]heptane-2- carboxylate | 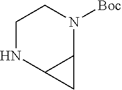 [SMILES: CC(C)(C)OC(=O)N1CCNC2CC21] | 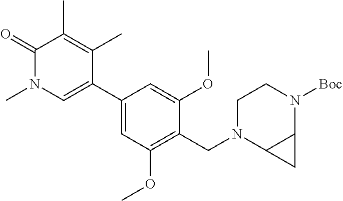 [SMILES: COc1cc(-c2cn(C)c(=O)c(C)c2C)cc(OC)c1CN1CCN(C(=O)OC(C)(C)C)C2CC21] | 484.4/90% |
| tert-butyl 4-(2,6-dimethoxy-4- (1,4,5-trimethyl-6-oxo-1,6- dihydropyridin-3- yl)benzyl)-2- isopropylpiperazine- 1-carboxylate | 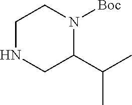 [SMILES: CC(C)C1CNCCN1C(=O)OC(C)(C)C] | 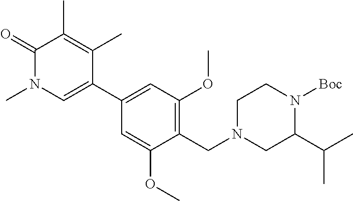 [SMILES: COc1cc(-c2cn(C)c(=O)c(C)c2C)cc(OC)c1CN1CCN(C(=O)OC(C)(C)C)C(C(C)C)C1] | 514.3/24% |
| tert-butyl 8-(2,6-dimethoxy-4- (1,4,5-trimethyl-6-oxo-1,6- dihydropyridin-3- yl)benzyl)-5,8- diazaspir[3.5]nonane-5- carboxylate | 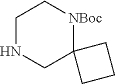 [SMILES: CC(C)(C)OC(=O)N1CCNCC12CCC2] | 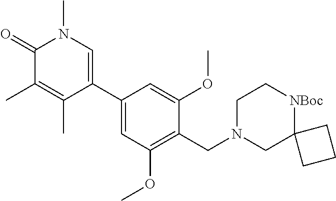 [SMILES: COc1cc(-c2cn(C)c(=O)c(C)c2C)cc(OC)c1CN1CCN(C(=O)OC(C)(C)C)C2(CCC2)C1] | 512.4/88% |
| tert-butyl (R)-4-(2,6-dimethoxy-4- (1,4,5-trimethyl-6-oxo-1,6- dihydropyridin-3-yl)benzyl)-3- ethylpiperazine-1-carboxylate | 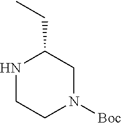 | 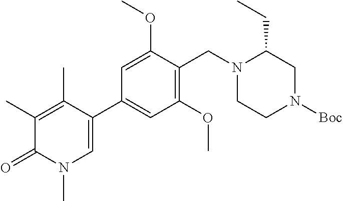 [SMILES: CC[C@@H]1CN(C(=O)OC(C)(C)C)CCN1Cc1c(OC)cc(-c2cn(C)c(=O)c(C)c2C)cc1OC] | 500.3/95% |
| tert-butyl (S)-4-(2,6-dimethoxy- 4- (1,4,5-trimethyl-6-oxo-1,6- dihydropyridin-3-yl)benzyl)-2- ethylpiperazine-1-carboxylate | 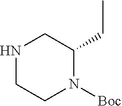 | 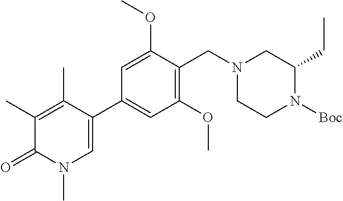 [SMILES: CC[C@H]1CN(Cc2c(OC)cc(-c3cn(C)c(=O)c(C)c3C)cc2OC)CCN1C(=O)OC(C)(C)C] | 500.4/95% |
| tert-butyl 5-((4-(2,6-dimethoxy- 4-(1,4,5-trimethyl-6-oxo-1,6- dihydropyridin-3-yl)benzyl)- 4,7-diazaspiro[2.5]octan-7- yl)methyl)-1-methyl-3,4- dihydro-isoquinoline-2(1H)- carboxylate | 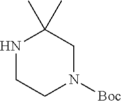 | 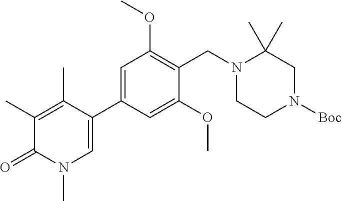 [SMILES: COc1cc(-c2cn(C)c(=O)c(C)c2C)cc(OC)c1CN1CCN(C(=O)OC(C)(C)C)CC1(C)C] | 500.4/83% |
| tert-butyl (2S,5S)-4-(2,6- dimethoxy-4-(1,4,5-trimethyl- 6-oxo-1,6- dihydropyridin-3-yl)benzyl)- 2,5-dimethylpiperazine-1- carboxylate | 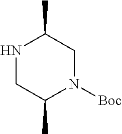 | 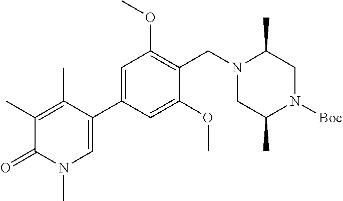 [SMILES: COc1cc(-c2cn(C)c(=O)c(C)c2C)cc(OC)c1CN1C[C@H](C)N(C(=O)OC(C)(C)C)C[C@@H]1C] | 500.4/92% |
| tert-butyl (2S,5R)-4-(2,6- dimethoxy-4-(1,4,5- trimethyl-6-oxo-1,6- dihydropyridin-3-yl)benzyl)- 2,5-dimethylpiperazine- 1-carboxylate | 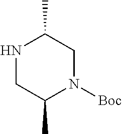 | 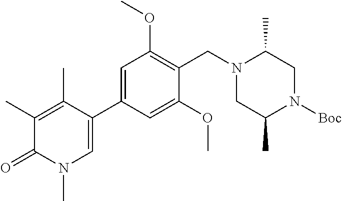 [SMILES: COc1cc(-c2cn(C)c(=O)c(C)c2C)cc(OC)c1CN1C[C@H](C)N(C(=O)OC(C)(C)C)C[C@H]1C] | 500.4/92% |
| 4-(2,6-dimethoxy-4-(1,4,5- trimethyl-6-oxo-1,6- dihydropyridin-3-yl)benzyl)- 3,3-dimethylpiperazin- 2-one | 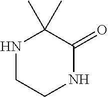 | 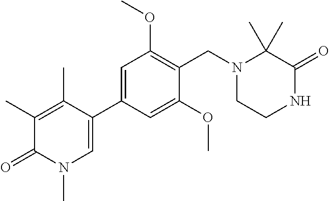 [SMILES: COc1cc(-c2cn(C)c(=O)c(C)c2C)cc(OC)c1CN1CCNC(=O)C1(C)C] | 414.2/86% |
| tert-butyl 4-(2,6-dimethoxy-4-(6- methyl-7-oxo-6,7-dihydro-1H- pyrazolo[3,4-c]pyridin-4- yl)benzyl)piperazine-1- carboxylate | 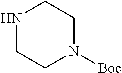 [SMILES: CC(C)(C)OC(=O)N1CCNCC1] | 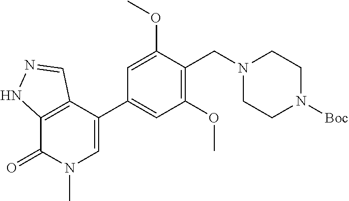 [SMILES: COc1cc(-c2cn(C)c(=O)c3[nH]ncc23)cc(OC)c1CN1CCN(C(=O)OC(C)(C)C)CC1] | 484.2/78% |
| tert-butyl 4-(2,6-dimethoxy- 4-(6-methyl-7-oxo-6,7- dihydro-1H-pyrazolo[3,4-c] pyridin-4-yl)benzyl)- 4,7-diazaspiro[2.5]octane-7- carboxylate | 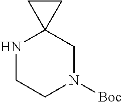 [SMILES: CC(C)(C)OC(=O)N1CCNC2(CC2)C1] | 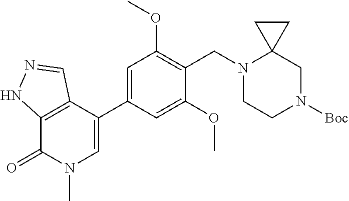 [SMILES: COc1cc(-c2cn(C)c(=O)c3[nH]ncc23)cc(OC)c1CN1CCN(C(=O)OC(C)(C)C)CC12CC2] | 510.2/70% |
| tert-butyl 7-(2,6-dimethoxy- 4-(6-methyl-7-oxo-6,7-dihydro- 1H-pyrazolo[3,4-c]pyridin- 4-yl)benzyl)-4,7- diazaspiro[2.5]octane-4- carboxylate | 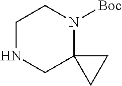 [SMILES: CC(C)(C)OC(=O)N1CCNCC12CC2] | 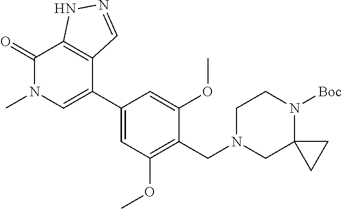 [SMILES: COc1cc(-c2cn(C)c(=O)c3[nH]ncc23)cc(OC)c1CN1CCN(C(=O)OC(C)(C)C)C2(CC2)C1] | 510.3/85% |
| tert-butyl 4-(2,6-dimethoxy- 4-(5-methyl-4-oxo-4,5- dihydrothieno[3,2-c]pyridin- 7-yl)benzyl)piperazine-1- carboxylate | 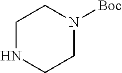 [SMILES: CC(C)(C)OC(=O)N1CCNCC1] | 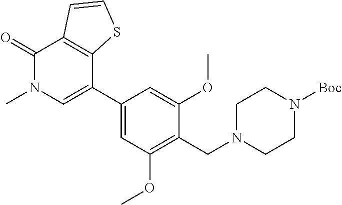 [SMILES: COc1cc(-c2cn(C)c(=O)c3ccsc23)cc(OC)c1CN1CCN(C(=O)OC(C)(C)C)CC1] | 500.1/72% |
| tert-butyl 7-(2,6-dimethoxy- 4-(5-methyl-4-oxo-4,5- dihydrothieno[3,2-c] pyridin-7-yl)benzyl)-4,7- diazaspiro[2.5]octane-4- carboxylate | 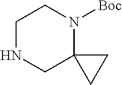 [SMILES: CC(C)(C)OC(=O)N1CCNCC12CC2] | 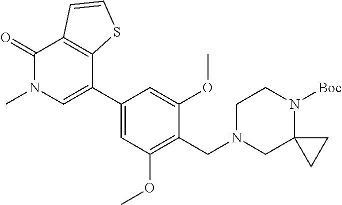 [SMILES: COc1cc(-c2cn(C)c(=O)c3ccsc23)cc(OC)c1CN1CCN(C(=O)OC(C)(C)C)C2(CC2)C1] | 526.3/85% |
| tert-butyl 4-(4-(1,5-dimethyl- 6-oxo-1,6-dihydropyridin- 3-yl)-2,6-dimethoxybenzyl) piperazine-1-carboxylate | 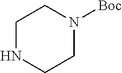 [SMILES: CC(C)(C)OC(=O)N1CCNCC1] | 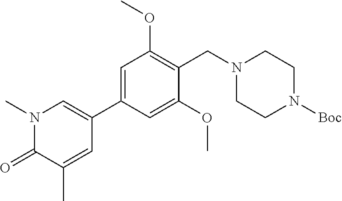 [SMILES: COc1cc(-c2cc(C)c(=O)n(C)c2)cc(OC)c1CN1CCN(C(=O)OC(C)(C)C)CC1] | 458.2/60% |

Table 3a: The following examples were prepared by general procedure 6 using carboxylic acid synthesized according to WO 2021/178920 and relevant commercially available mono protected diamine specified.

| | | LCMS m/z |
|---|---|---|
| Amine (Y) | Product Structure/Name | [M + H]⁺/Yield |
| 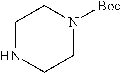 [SMILES: CC(C)(C)OC(=O)N1CCNCC1] | 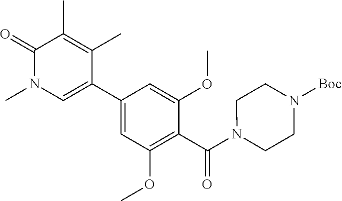 [SMILES: COc1cc(-c2cn(C)c(=O)c(C)c2C)cc(OC)c1C(=O)N1CCN(C(=O)OC(C)(C)C)CC1] | 486.4/52% |
| | tert-butyl 4-(2,6-dimethoxy-4-(1,4,5-trimethyl-6-oxo-1,6- | |
| | dihydropyridin-3-yl)benzoyl)piperazine-1-carboxylate | |
| 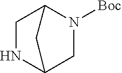 [SMILES: CC(C)(C)OC(=O)N1CC2CC1CN2] | 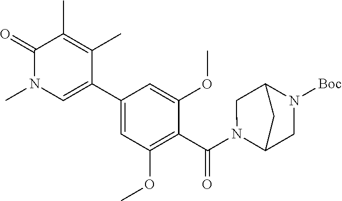 [SMILES: COc1cc(-c2cn(C)c(=O)c(C)c2C)cc(OC)c1C(=O)N1CC2CC1CN2C(=O)OC(C)(C)C] | 498.2/43% |
| | tert-butyl 5-(2,6-dimethoxy-4-(1,4,5-trimethyl-6-oxo-1,6- | |
| | dihydropyridin-3-yl)benzoyl)-2,5-diazabicyclo[2.2.1]heptane-2- | |
| | carboxylate | |
| 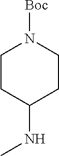 | 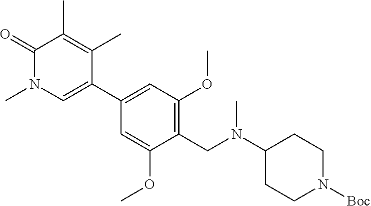 [SMILES: COc1cc(-c2cn(C)c(=O)c(C)c2C)cc(OC)c1CN(C)C1CCN(C(=O)OC(C)(C)C)CC1] | 514.2/26% |
| | tert-butyl 4-((2,6-dimethoxy-4-(1,4,5-trimethyl-6-oxo-1,6- | |
| | dihydropyridin-3-yl)benzyl)(methyl)amino)piperidine-1- | |
| | carboxylate | |
| 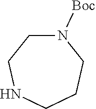 [SMILES: CC(C)(C)OC(=O)N1CCCNCC1] | 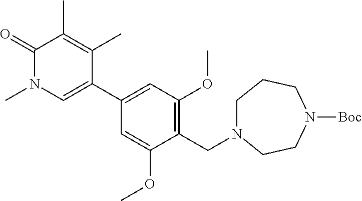 [SMILES: COc1cc(-c2cn(C)c(=O)c(C)c2C)cc(OC)c1CN1CCCN(C(=O)OC(C)(C)C)CC1] | 500.4/36% |
| | tert-butyl 4-(2,6-dimethoxy-4-(1,4,5-trimethyl-6-oxo-1,6- | |
| | dihydropyridin-3-yl)benzoyl)-1,4-diazepane-1-carboxylate | |
| 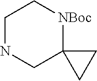 [SMILES: CC(C)(C)OC(=O)N1CCNCC12CC2] | 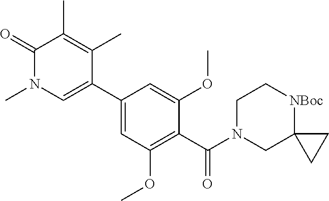 [SMILES: COc1cc(-c2cn(C)c(=O)c(C)c2C)cc(OC)c1C(=O)N1CCN(C(=O)OC(C)(C)C)C2(CC2)C1] | 512.3/97% |
| | tert-butyl 7-(2,6-dimethoxy-4-(1,4,5-trimethyl-6-oxo-1,6- | |
| | dihydropyridin-3-yl)benzoyl)-4,7-diazaspiro[2.5]octane-4- | |
| | carboxylate | |
| 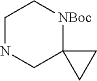 [SMILES: CC(C)(C)OC(=O)N1CCNCC12CC2] | 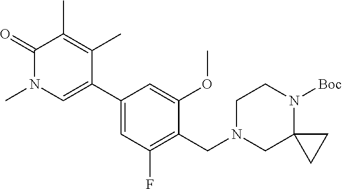 [SMILES: COc1cc(-c2cn(C)c(=O)c(C)c2C)cc(F)c1CN1CCN(C(=O)OC(C)(C)C)C2(CC2)C1] | 486.2/92% |
| | tert-butyl 7-(2-fluoro-6-methoxy-4-(1,4,5-trimethyl-6-oxo-1,6- | |
| | dihydropyridin-3-yl)benzyl)-4,7-diazaspiro[2.5]octane-4- | |
| | carboxylate | |

Table 4a: The following examples were prepared by general procedure 2 using the starting materials synthesized in Table 3a.

TABLE 4a

| | | LCMS m/z |
|---|---|---|
| Name | Product Structure | [M + H]⁺/Yield |
| 4-(4-((1,4-diazepan-1-yl)methyl)-3,5- dimethoxyphenyl)-2-methyl-2,7-naphthyridin- 1(2H)-one | 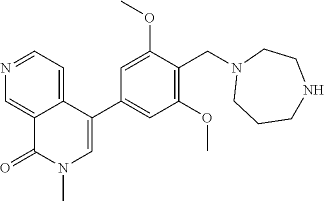 [SMILES: COc1cc(-c2cn(C)c(=O)c3cnccc23)cc(OC)c1CN1CCCNCC1] | 409.6/98% |
| 4-(3,5-dimethoxy-4-((methyl(2- (methylamino)ethyl)amino)methyl)phenyl)-2- methyl-2,7-naphthyridin-1(2H)-one | 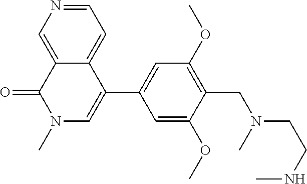 [SMILES: CNCCN(C)Cc1c(OC)cc(-c2cn(C)c(=O)c3cnccc23)cc1OC] | 397.6/97% |
| 4-(4-((2,5-diazabicyclo[2.2.1]heptan-2- yl)methyl)-3,5-dimethoxyphenyl)-2-methyl- 2,7-naphthyridin-1(2H)-one | 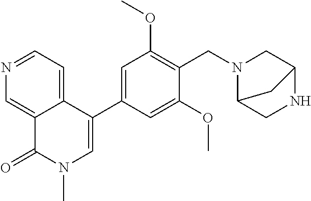 [SMILES: COc1cc(-c2cn(C)c(=O)c3cnccc23)cc(OC)c1CN1CC2CC1CN2] | 407.0/97% |
| 4-(4-((3,6-diazabicyclo[3.1.1]heptan-6- yl)methyl)-3,5-dimethoxyphenyl)-2-methyl- 2,7-naphthyridin-1(2H)-one | 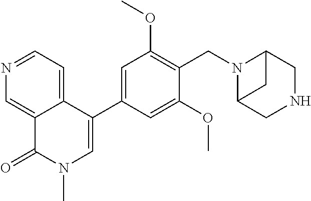 [SMILES: COc1cc(-c2cn(C)c(=O)c3cnccc23)cc(OC)c1CN1C2CNCC1C2] | 407.0/97% |
| 4-(4-((3,6-diazabicyclo[3.1.1]heptan-3- yl)methyl)-3,5-dimethoxyphenyl)-2-methyl- 2,7-naphthyridin-1(2H)-one | 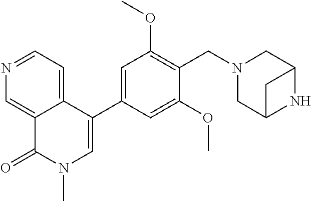 [SMILES: COc1cc(-c2cn(C)c(=O)c3cnccc23)cc(OC)c1CN1CC2CC(C1)N2] | 407.0/96% |
| 4-(4-((3,8-diazabicyclo[3.2.1]octan-3- yl)methyl)-3,5-dimethoxyphenyl)-2-methyl- 2,7-naphthyridin-1(2H)-one | 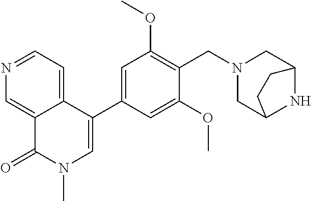 [SMILES: COc1cc(-c2cn(C)c(=O)c3cnccc23)cc(OC)c1CN1CC2CCC(C1)N2] | 421.1/98% |
| 5-(3,5-dimethoxy-4-(piperazin-1- ylmethyl)phenyl)-1,3,4-trimethylpyridin-2(1H)- one | 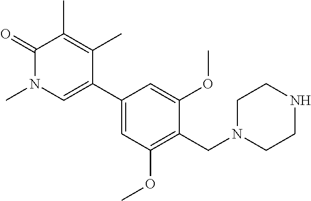 [SMILES: COc1cc(-c2cn(C)c(=O)c(C)c2C)cc(OC)c1CN1CCNCC1] | 372.4/94% |
| 5-(4-((2,5-diazabicyclo[2.2.1]heptan-2- yl)methyl)-3,5-dimethoxyphenyl)-1,3,4- trimethylpyridin-2(1H)-one | 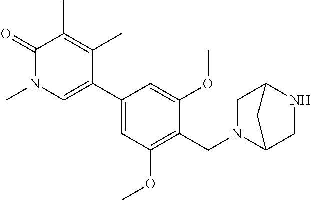 [SMILES: COc1cc(-c2cn(C)c(=O)c(C)c2C)cc(OC)c1CN1CC2CC1CN2] | 384.3/94% |
| 5-(4-((3,6-diazabicyclo[3.1.1]heptan-3- yl)methyl)-3,5-dimethoxyphenyl)-1,3,4- trimethylpyridin-2(1H)-one | 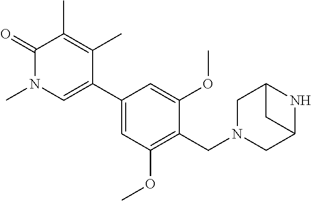 [SMILES: COc1cc(-c2cn(C)c(=O)c(C)c2C)cc(OC)c1CN1CC2CC(C1)N2] | 384.4/87% |
| 5-(4-((3,6-diazabicyclo[3.1.1]heptan-6- yl)methyl)-3,5-dimethoxyphenyl)-1,3,4- trimethylpyridin-2(1H)-one | 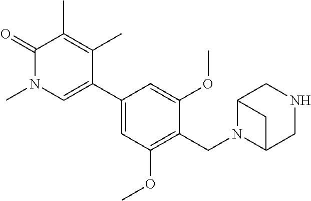 [SMILES: COc1cc(-c2cn(C)c(=O)c(C)c2C)cc(OC)c1CN1C2CNCC1C2] | 384.4/94% |
| 5-(4-((3,8-diazabicyclo[3.2.1]octan-3- yl)methyl)-3,5-dimethoxyphenyl)-1,3,4- trimethylpyridin-2(1H)-one | 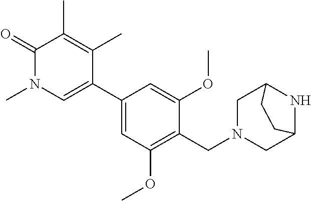 [SMILES: COc1cc(-c2cn(C)c(=O)c(C)c2C)cc(OC)c1CN1CC2CCC(C1)N2] | 398.5/87% |
| 5-(4-((1,4-diazepan-1-yl)methyl)-3,5- dimethoxyphenyl)-1,3,4-trimethylpyridin- 2(1H)-one | 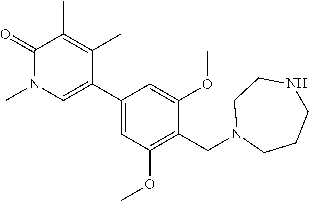 [SMILES: COc1cc(-c2cn(C)c(=O)c(C)c2C)cc(OC)c1CN1CCCNCC1] | 386.3/92% |
| 5-(3,5-dimethoxy-4-((methyl(piperidin-4- yl)amino)methyl)phenyl)-1,3,4- trimethylpyridin-2(1H)-one | 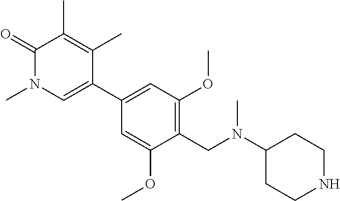 [SMILES: COc1cc(-c2cn(C)c(=O)c(C)c2C)cc(OC)c1CN(C)C1CCNCC1] | 400.2/94% |
| 5-(4-((4,7-diazaspiro[2.5]octan-7-yl)methyl)- 3,5-dimethoxyphenyl)-1,3,4-trimethylpyridin- 2(1H)-one | 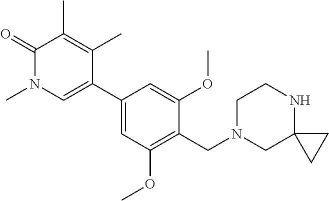 [SMILES: COc1cc(-c2cn(C)c(=O)c(C)c2C)cc(OC)c1CN1CCNC2(CC2)C1] | 398.3/93% |
| 5-(4-((4,7-diazaspiro[2.5]octan-4-yl)methyl)- 3,5-dimethoxyphenyl)-1,3,4-trimethylpyridin- 2(1H)-one | 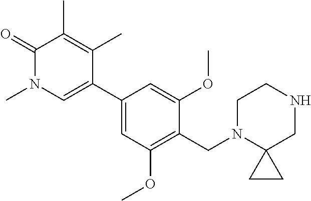 [SMILES: COc1cc(-c2cn(C)c(=O)c(C)c2C)cc(OC)c1CN1CCNCC12CC2] | 398.4/99% |
| 5-(4-((3,9-diazaspiro[5.5]undecan-3- yl)methyl)-3,5-dimethoxyphenyl)-1,3,4- trimethylpyridin-2(1H)-one | 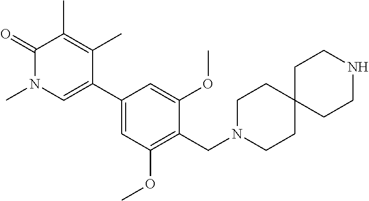 [SMILES: COc1cc(-c2cn(C)c(=O)c(C)c2C)cc(OC)c1CN1CCC2(CCNCC2)CC1] | 440.5/91% |
| 5-(3,5-dimethoxy-4- ((methylamino)methyl)phenyl)-1,3,4- trimethylpyridin-2(1H)-one | 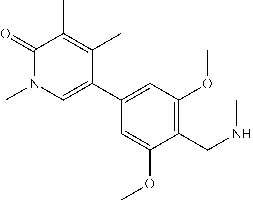 | 317.5/87% |
| 5-(4-(((2S,5R)-2,5-dimethylpiperazin-1- yl)methyl)-3,5-dimethoxyphenyl)-1,3,4- trimethylpyridin-2(1H)-one | 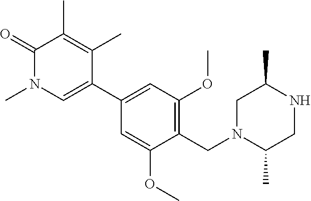 [SMILES: COc1cc(-c2cn(C)c(=O)c(C)c2C)cc(OC)c1CN1C[C@@H](C)NC[C@@H]1C] | 400.2/90% |
| 5-(4-((3,3-dimethylpiperazin-1-yl)methyl)-3,5- dimethoxyphenyl)-1,3,4-trimethylpyridin- 2(1H)-one | 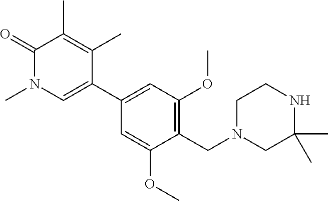 | 400.4/81% |
| 5-(4-(((3S,5S)-3,5-dimethylpiperazin-1- yl)methyl)-3,5-dimethoxyphenyl)-1,3,4- trimethylpyridin-2(1H)-one | 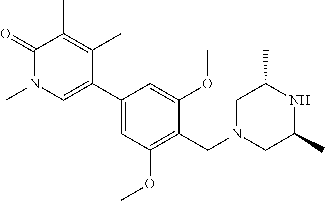 | 400.3/89% |
| 5-(4-((2,5-diazabicyclo[2.2.2]octan-2- yl)methyl)-3,5-dimethoxyphenyl)-1,3,4- trimethylpyridin-2(1H)-one | 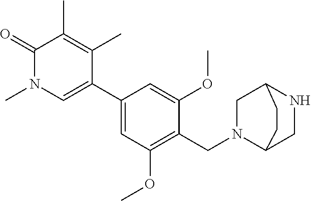 [SMILES: COc1cc(-c2cn(C)c(=O)c(C)c2C)cc(OC)c1CN1CC2CCC1CN2] | 398.2/83% |
| (R)-5-(3,5-dimethoxy-4-((3-methylpiperazin- 1-yl)methyl)phenyl)-1,3,4-trimethylpyridin- 2(1H)-one | 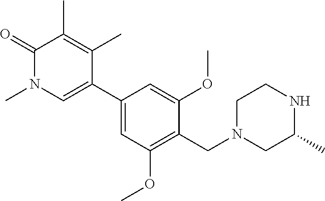 | 386.2/92% |
| (S)-5-(3,5-dimethoxy-4-((3-methylpiperazin- 1-yl)methyl)phenyl)-1,3,4-trimethylpyridin- 2(1H)-one | 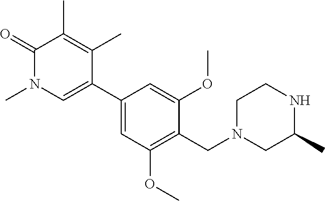 | 386.2/87% |
| 5-(4-(((2S,6R)-2,6-dimethylpiperazin-1- yl)methyl)-3,5-dimethoxyphenyl)-1,3,4- trimethylpyridin-2(1H)-one | 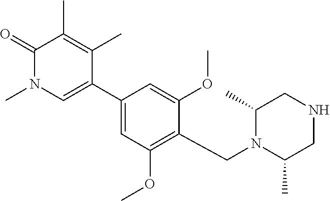 [SMILES: COc1cc(-c2cn(C)c(=O)c(C)c2C)cc(OC)c1CN1[C@H](C)CNC[C@@H]1C] | 400.2/85% |
| 5-(4-(((2R,5R)-2,5-dimethylpiperazin-1- yl)methyl)-3,5-dimethoxyphenyl)-1,3,4- trimethylpyridin-2(1H)-one | 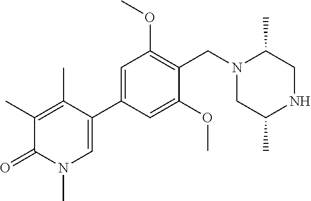 [SMILES: COc1cc(-c2cn(C)c(=O)c(C)c2C)cc(OC)c1CN1C[C@@H](C)NC[C@H]1C] | 400.4/93% |
| (S)-5-(4-((3-ethylpiperazin-1-yl)methyl)-3,5- dimethoxyphenyl)-1,3,4-trimethylpyridin- 2(1H)-one | 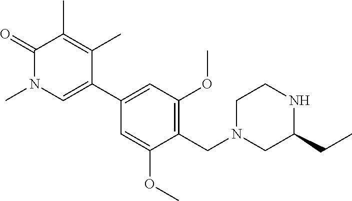 [SMILES: CC[C@H]1CN(Cc2c(OC)cc(-c3cn(C)c(=O)c(C)c3C)cc2OC)CCN1] | 400.2/81% |
| (S)-5-(4-((2-ethylpiperazin-1-yl)methyl)-3,5- dimethoxyphenyl)-1,3,4-trimethylpyridin- 2(1H)-one | 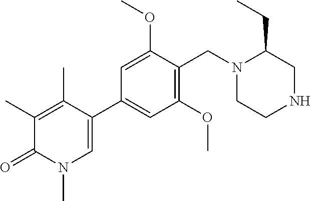 [SMILES: CC[C@H]1CNCCN1Cc1c(OC)cc(-c2cn(C)c(=O)c(C)c2C)cc1OC] | 400.3/96% |
| 5-(4-((2,5-diazabicyclo[4.1.0]heptan-2- yl)methyl)-3,5-dimethoxyphenyl)-1,3,4- trimethylpyridin-2(1H)-one | 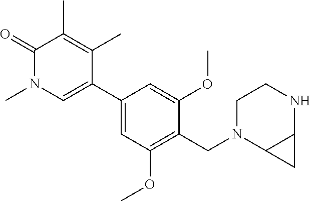 [SMILES: COc1cc(-c2cn(C)c(=O)c(C)c2C)cc(OC)c1CN1CCNC2CC21] | 384.3/78% |
| 5-(4-((3-isopropylpiperazin-1-yl)methyl)-3,5- dimethoxyphenyl)-1,3,4-trimethylpyridin- 2(1H)-one | 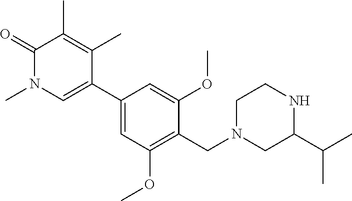 [SMILES: COc1cc(-c2cn(C)c(=O)c(C)c2C)cc(OC)c1CN1CCNC(C(C)C)C1] | 414.2/81% |
| 5-(4-((5,8-diazaspiro[3.5]nonan-8-yl)methyl)- 3,5-dimethoxyphenyl)-1,3,4-trimethylpyridin- 2(1H)-one | 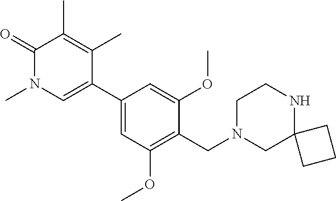 [SMILES: COc1cc(-c2cn(C)c(=O)c(C)c2C)cc(OC)c1CN1CCNC2(CCC2)C1] | 412.3/83% |
| (R)-5-(4-((2-ethylpiperazin-1-yl)methyl)-3,5- dimethoxyphenyl)-1,3,4-trimethylpyridin- 2(1H)-one | 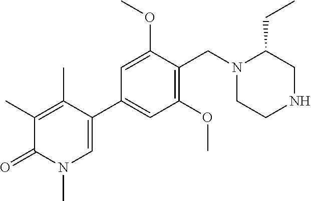 [SMILES: CC[C@@H]1CNCCN1Cc1c(OC)cc(-c2cn(C)c(=O)c(C)c2C)cc1OC] | 400.2/79% |
| (S)-5-(4-((3-ethylpiperazin-1-yl)methyl)-3,5- dimethoxyphenyl)-1,3,4-trimethylpyridin- 2(1H)-one | 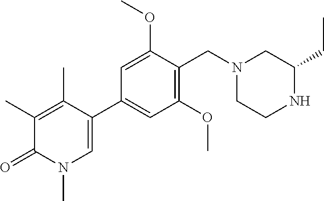 [SMILES: CC[C@H]1CN(Cc2c(OC)cc(-c3cn(C)c(=O)c(C)c3C)cc2OC)CCN1] | 400.2/81% |
| 5-(4-((2,2-dimethylpiperazin-1-yl)methyl)-3,5- dimethoxyphenyl)-1,3,4-trimethylpyridin- 2(1H)-one | 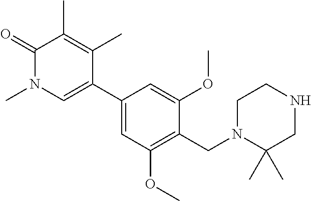 [SMILES: COc1cc(-c2cn(C)c(=O)c(C)c2C)cc(OC)c1CN1CCNCC1(C)C] | 400.2/92% |
| 5-(4-(((2S,5S)-2,5-dimethylpiperazin-1- yl)methyl)-3,5-dimethoxyphenyl)-1,3,4- trimethylpyridin-2(1H)-one | 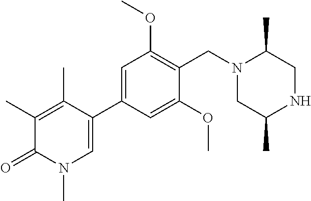 [SMILES: COc1cc(-c2cn(C)c(=O)c(C)c2C)cc(OC)c1CN1C[C@H](C)NC[C@@H]1C] | 400.4/92% |
| 5-(4-(((2R,5S)-2,5-dimethylpiperazin-1- yl)methyl)-3,5-dimethoxyphenyl)-1,3,4- trimethylpyridin-2(1H)-one | 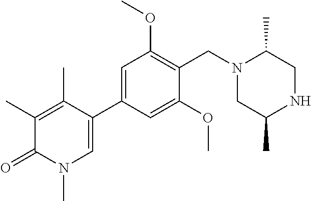 [SMILES: COc1cc(-c2cn(C)c(=O)c(C)c2C)cc(OC)c1CN1C[C@H](C)NC[C@H]1C] | 400.4/88% |
| 4-(3,5-dimethoxy-4-(piperazin-1- ylmethyl)phenyl)-6-methyl-1,6-dihydro-7H- pyrazolo[3,4-c]pyridin-7-one | 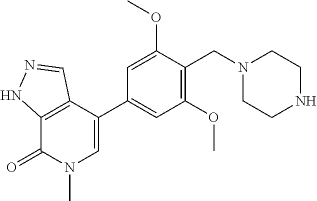 [SMILES: COc1cc(-c2cn(C)c(=O)c3[nH]ncc23)cc(OC)c1CN1CCNCC1] | 384.2/79% |
| 4-(4-((4,7-diazaspiro[2.5]octan-4-yl)methyl)- 3,5-dimethoxyphenyl)-6-methyl-1,6-dihydro- 7H-pyrazolo[3,4-c]pyridin-7-one | 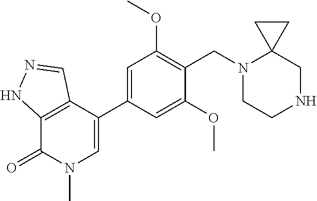 [SMILES: COc1cc(-c2cn(C)c(=O)c3[nH]ncc23)cc(OC)c1CN1CCNCC12CC2] | 410.4/99% |
| 4-(4-((4,7-diazaspiro[2.5]octan-7-yl)methyl)- 3,5-dimethoxyphenyl)-6-methyl-1,6-dihydro- 7H-pyrazolo[3,4-c]pyridin-7-one | 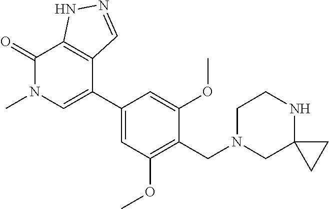 [SMILES: COc1cc(-c2cn(C)c(=O)c3[nH]ncc23)cc(OC)c1CN1CCNC2(CC2)C1] | 410.2/87% |
| 7-(3,5-dimethoxy-4-(piperazin-1- ylmethyl)phenyl)-5-methylthieno[3,2- c]pyridin-4(5H)-one | 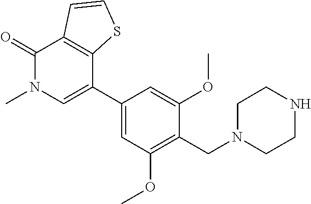 [SMILES: COc1cc(-c2cn(C)c(=O)c3ccsc23)cc(OC)c1CN1CCNCC1] | 400.2/89% |
| 7-(4-((4,7-diazaspiro[2.5]octan-7-yl)methyl)- 3,5-dimethoxyphenyl)-5-methylthieno[3,2- c]pyridin-4(5H)-one | 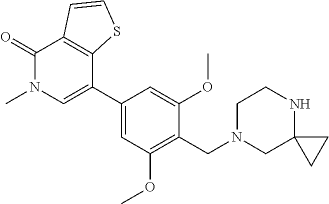 [SMILES: COc1cc(-c2cn(C)c(=O)c3ccsc23)cc(OC)c1CN1CCNC2(CC2)C1] | 426.2/85% |
| 5-(3,5-dimethoxy-4-(piperazin-1- ylmethyl)phenyl)-1,3-dimethylpyridin-2(1H)- one | 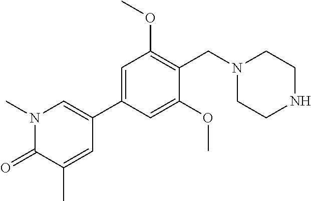 [SMILES: COc1cc(-c2cc(C)c(=O)n(C)c2)cc(OC)c1CN1CCNCC1] | 358.3/71% |
| 5-(3,5-dimethoxy-4-(piperazine-1- carbonyl)phenyl)-1,3,4-trimethylpyridin- 2(1H)-one | 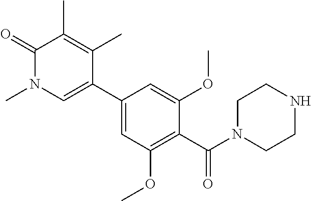 [SMILES: COc1cc(-c2cn(C)c(=O)c(C)c2C)cc(OC)c1C(=O)N1CCNCC1] | 386.5/87% |
| 5-(4-(2,5-diazabicyclo[2.2.1]heptane-2- carbonyl)-3,5-dimethoxyphenyl)-1,3,4- trimethylpyridin-2(1H)-one | 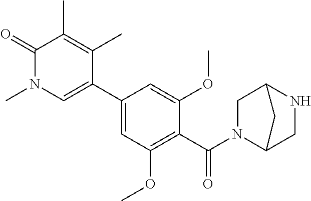 [SMILES: COc1cc(-c2cn(C)c(=O)c(C)c2C)cc(OC)c1C(=O)N1CC2CC1CN2] | 398.4/85% |
| 2,6-dimethoxy-N-methyl-N-(piperidin-4-yl)-4- (1,4,5-trimethyl-6-oxo-1,6-dihydropyridin-3- yl)benzamide | 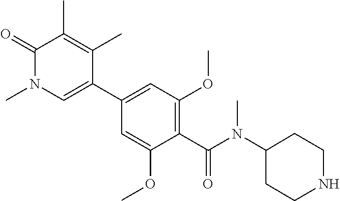 [SMILES: COc1cc(-c2cn(C)c(=O)c(C)c2C)cc(OC)c1C(=O)N(C)C1CCNCC1] | 414.2/83% |
| 5-(4-(2,5-diazabicyclo[2.2.1]heptane-2- carbonyl)-3,5-dimethoxyphenyl)-1,3,4- trimethylpyridin-2(1H)-one | 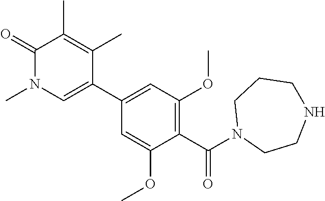 [SMILES: COc1cc(-c2cn(C)c(=O)c(C)c2C)cc(OC)c1C(=O)N1CCCNCC1] | 400.6/92% |
| 2,6-dimethoxy-N-methyl-N-(piperidin-4-yl)-4- (1,4,5-trimethyl-6-oxo-1,6-dihydropyridin-3- yl)benzamide | 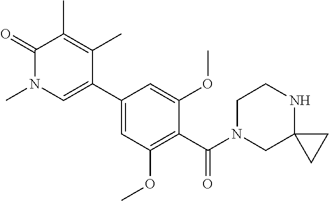 [SMILES: COc1cc(-c2cn(C)c(=O)c(C)c2C)cc(OC)c1C(=O)N1CCNC2(CC2)C1] | 412.2/88% |
| 5-(4-((4,7-diazaspiro[2.5]octan-7-yl)methyl)- 3-fluoro-5-methoxyphenyl)-1,3,4- trimethylpyridin-2(1H)-one | 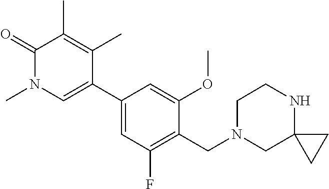 [SMILES: COc1cc(-c2cn(C)c(=O)c(C)c2C)cc(F)c1CN1CCNC2(CC2)C1] | 386.2/87% |

Table 4b: The following intermediates were made according to General Procedure 28.

| | | Mass | |
|---|---|---|---|
| | | isolated, | |
| Compound | Name | yield | LCMS m/z [M + H]⁺ |
| 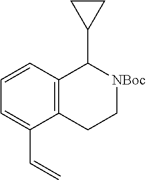 [SMILES: C=Cc1cccc2c1CCN(C(=O)OC(C)(C)C)C2C1CC1] | tert-butyl 1- cyclopropyl-5-vinyl- 3,4- dihydroisoquinoline- 2(1H)-carboxylate | 730 mg, 75% yield | 200.4 [M + H − Boc]⁺ |
| 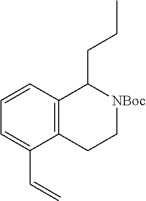 [SMILES: C=Cc1cccc2c1CCN(C(=O)OC(C)(C)C)C2CCC] | tert-butyl 1-propyl-5- vinyl-3,4- dihydroisoquinoline- 2(1H)-carboxylate | 850 mg, 77% yield | 202.2 [M + H − Boc]⁺ |
| 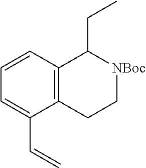 [SMILES: C=Cc1cccc2c1CCN(C(=O)OC(C)(C)C)C2CC] | tert-butyl 1-ethyl-5- vinyl-3,4- dihydroisoquinoline- 2(1H)-carboxylate | 650 mg, 78% yield | 232.2 [M + H − 56]⁺. |
| 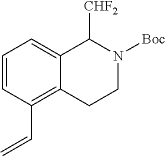 [SMILES: C=Cc1cccc2c1CCN(C(=O)OC(C)(C)C)C2C(F)F] | tert-butyl 1- (difluoromethyl)-5- vinyl-3,4- dihydroisoquinoline- 2(1H)-carboxylate | 800 mg, 94% yield | 210.4 [M + H − Boc]⁺ |
| 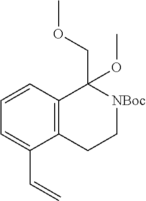 | tert-butyl 1- (methoxymethyl)-5- vinyl-3,4- dihydroisoquinoline- 2(1H)-carboxylate | 600 mg, 88% yield | 246.2 [M + H − 56]⁺. |

Table 4c: The following intermediates were made according to General Procedure 29 from the respective olefins in Table 4b:

| | | | Mass | |
|---|---|---|---|---|
| | | | isolated, | |
| Compound | Name | Procedure | yield | LCMS m/z [M + H]⁺ |
| 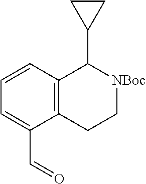 [SMILES: CC(C)(C)OC(=O)N1CCc2c(C=O)cccc2C1C1CC1] | tert-butyl 1- cyclopropyl-5- formyl-3,4- dihydroisoquinoline- 2(1H)-carboxylate | 29 | 500 mg, 71% yield | 202.0 [M + H − Boc]⁺ |
| 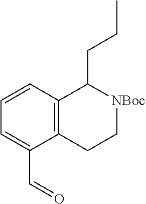 [SMILES: CCCC1c2cccc(C=O)c2CCN1C(=O)OC(C)(C)C] | tert-butyl 5-formyl- 1-propyl-3,4- dihydroisoquinoline- 2(1H)-carboxylate | 29 | 750 mg, 88% yield | 204.2 [M + H − Boc]⁻ |
| 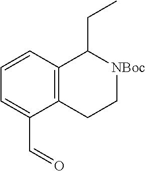 [SMILES: CCC1c2cccc(C=O)c2CCN1C(=O)OC(C)(C)C] | tert-butyl 1-ethyl-5- formyl-3,4- dihydroisoquinoline- 2(1H)-carboxylate | 29 | 250 mg, 38% yield | 234.2 [M + H − 56]⁺ |
| 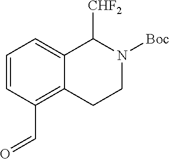 [SMILES: CC(C)(C)OC(=O)N1CCc2c(C=O)cccc2C1C(F)F] | tert-butyl 1- (difluoromethyl)-5- formyl-3,4- dihydroisoquinoline- 2(1H)-carboxylate | 29 | 300 mg, 60% yield | 212.4 [M + H − Boc]⁺ |
| 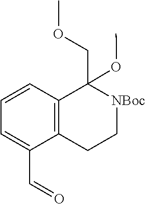 | tert-butyl 5-formyl- 1-(methoxymethyl)- 3,4- dihydroisoquinoline- 2(1H)-carboxylate | 29 | 480 mg, 94% yield | 204.2 [M + H − Boc]⁺ |

Table 5a: The following examples were prepared by general procedure 14 using the relevant amine (Table 4a) and relevant aldehyde from Table 4c or as defined below.

| | |
|---|---|
| 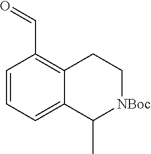 [SMILES: CC1c2cccc(C=O)c2CCN1C(=O)OC(C)(C)C] | 1 |
| 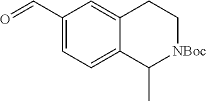 [SMILES: CC1c2ccc(C=O)cc2CCN1C(=O)OC(C)(C)C] | 2 |
| 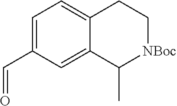 [SMILES: CC1c2cc(C=O)ccc2CCN1C(=O)OC(C)(C)C] | 3 |
| 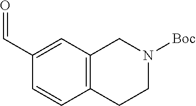 [SMILES: CC(C)(C)OC(=O)N1CCc2ccc(C=O)cc2C1] | 4 |
| as described in | |
| WO2019/99578 | |
| 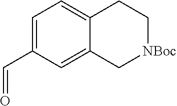 [SMILES: CC(C)(C)OC(=O)N1CCc2ccc(C=O)cc2C1] | 5 |
| as described in | |
| US2003/09230 | |
| 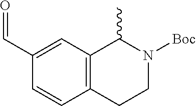 [SMILES: CC1c2cc(C=O)ccc2CCN1C(=O)OC(C)(C)C] | 6 |
| enantiomer 1 (unknown | |
| absolute configuration) | |
| 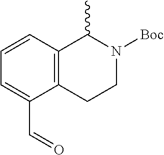 [SMILES: CC1c2cccc(C=O)c2CCN1C(=O)OC(C)(C)C] | 7 |
| enantiomer 1 (unknown | |
| absolute configuration) | |
| 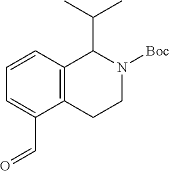 [SMILES: CC(C)C1c2cccc(C=O)c2CCN1C(=O)OC(C)(C)C] | 8 |
| 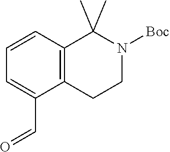 [SMILES: CC(C)(C)OC(=O)N1CCc2c(C=O)cccc2C1(C)C] | 9 |
| 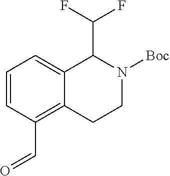 [SMILES: CC(C)(C)OC(=O)N1CCc2c(C=O)cccc2C1C(F)F] | 10 |
| 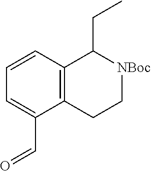 [SMILES: CCC1c2cccc(C=O)c2CCN1C(=O)OC(C)(C)C] | 11 |
| 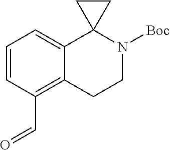 [SMILES: CC(C)(C)OC(=O)N1CCc2c(C=O)cccc2C12CC2] | 12 |
| 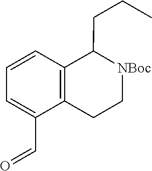 [SMILES: CCCC1c2cccc(C=O)c2CCN1C(=O)OC(C)(C)C] | 13 |
| 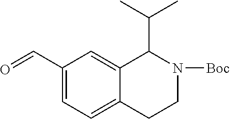 [SMILES: CC(C)C1c2cc(C=O)ccc2CCN1C(=O)OC(C)(C)C] | 14 |
| 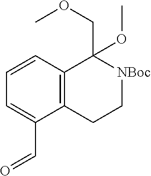 [SMILES: COCC1(OC)c2cccc(C=O)c2CCN1C(=O)OC(C)(C)C] | 15 |

2 and 3 are made in an analogous manner to 1 (intermediate W-22). The routes to the precursor Br-THIQs starting materials of these compounds is provided in WO2022/129925.

7—Racemic tert-butyl 5-bromo-1-methyl-3,4-dihydroisoquinoline-2(1H)-carboxylate (50 g, as prepared in WO2022/129925) was purified by asymmetric SFC:

| Column | WHELK RR-(250*30 mm), 5 μm | |
|---|---|---|
| Mobile Phase | CO2: 0.5% isopropylamine in | |
| | MeOH:MeCN [90:10] | |
| Flow rate | 5 | mL/min |
| Back pressure | 100 | bar |
| Wavelength | 220 | nm |
| Cycle time | 6 | min |

Isolated Peak-1: 22 g, LCMS m/z=226.2 [M-Boc]+ and Peak-2: 22 g, LCMS r L/z=226.2 [M-Boc]+

The synthesis of enantiomer 1 of tert-butyl 5-formyl-1-methyl-3,4-dihydroisoquinoline-2(1H)-carboxylate was then completed following the same protocols as used for the preparation of Intermediate W-17 and Intermediate W-1-8

8—Derived from THIQ-11. Derivatisation to the aldehyde was done in the same way as for compounds in table 4b and 4c.

9—Derived from THIQ-6. Derivatisation to the aldehyde was done in the same way as for compounds in table 4b and 4c.

14 prepared in an analogous manner to Intermediate WM-14, with the iPr THIQ starting material.

TABLE 5a

| | | | LCMS m/z |
|---|---|---|---|
| | | | [M + |
| | | | H]⁺/ |
| Name | Y | Product Structure | Yield |
| tert-butyl 5-((4- (2,6-dimethoxy-4-(2-methyl- 1-oxo-1,2-dihydro-2,7- naphthyridin-4- yl)benzyl)piperazin-1- yl)methyl)-1-methyl- 3,4-dihydroisoquinoline- 2(1H)-carboxylate | 1 | 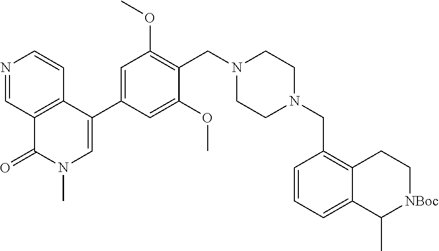 [SMILES: COc1cc(-c2cn(C)c(=O)c3cnccc23)cc(OC)c1CN1CCN(Cc2cccc3c2CCN(C(=O)OC(C)(C)C)C3C)CC1] | 654.7/74% |
| tert-butyl 5-((4- (2,6-dimethoxy-4-(1,4,5- trimethyl-6-oxo-1,6- dihydropyridin-3- yl)benzyl)piperazin-1-yl) methyl)-1-methyl- 3,4-dihydroisoquinoline- 2(1H)-carboxylate | 1 | 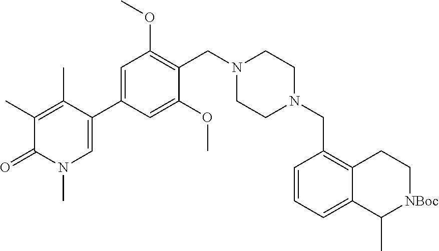 [SMILES: COc1cc(-c2cn(C)c(=O)c(C)c2C)cc(OC)c1CN1CCN(Cc2cccc3c2CCN(C(=O)OC(C)(C)C)C3C)CC1] | 631.1/64% |
| tert-butyl 5-((4-(2,6- dimethoxy-4-(2-methyl-1- oxo-1,2-dihydro-2,7- naphthyridin-4-yl)benzyl)- 1,4-diazepan-1-yl)methyl)- 1-methyl- 3,4-dihydroisoquinoline- 2(1H)-carboxylate | 1 | 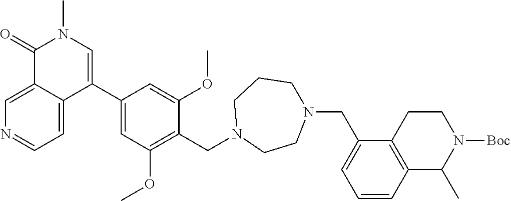 [SMILES: COc1cc(-c2cn(C)c(=O)c3cnccc23)cc(OC)c1CN1CCCN(Cc2cccc3c2CCN(C(=O)OC(C)(C)C)C3C)CC1] | 668.2/53% |
| tert-butyl 5-[[[2,6- dimethoxy-4-(2-methyl-1- oxo-2,7-naphthyridin-4-yl) phenyl]methyl-methyl- amino]methyl]-1-methyl- 3,4-dihydro-1H- isoquinoline-2-carboxylate | 1 | 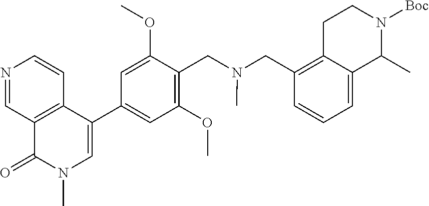 [SMILES: COc1cc(-c2cn(C)c(=O)c3cnccc23)cc(OC)c1CN(C)Cc1cccc2c1CCN(C(=O)OC(C)(C)C)C2C] | 599.0/34% |
| tert-butyl 6-[[[2,6- dimethoxy-4-(2-methyl-1- oxo-2,7-naphthyridin-4-yl) phenyl]methyl-methyl- amino]methyl]-1-methyl- 3,4-dihydro-1H- isoquinoline-2- carboxylate | 2 | 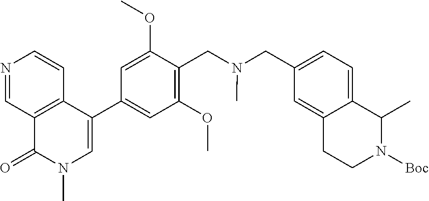 [SMILES: COc1cc(-c2cn(C)c(=O)c3cnccc23)cc(OC)c1CN(C)Cc1ccc2c(c1)CCN(C(=O)OC(C)(C)C)C2C] | 599.0/67% |
| tert-butyl 7-[[[2,6- dimethoxy-4-(2-methyl-1- oxo-2,7-naphthyridin-4-yl) phenyl]methyl-methyl- amino]methyl]-1-methyl- 3,4-dihydro-1H- isoquinoline-2- carboxylate | 3 | 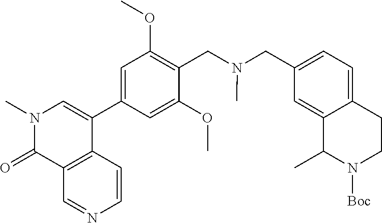 [SMILES: COc1cc(-c2cn(C)c(=O)c3cnccc23)cc(OC)c1CN(C)Cc1ccc2c(c1)C(C)N(C(=O)OC(C)(C)C)CC2] | 599.0/48% |
| tert-butyl 5-(((2-((2,6- dimethoxy-4-(2-methyl- 1-oxo-1,2-dihydro-2,7- naphthyridin-4-yl)benzyl) (methyl)amino)ethyl) (methyl)amino)methyl)-1- methyl-3,4-dihydroisoquinoline- 2(1H)-carboxylate | 1 | 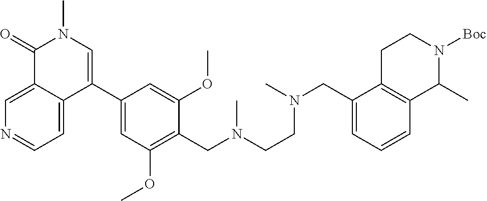 [SMILES: COc1cc(-c2cn(C)c(=O)c3cnccc23)cc(OC)c1CN(C)CCN(C)Cc1cccc2c1CCN(C(=O)OC(C)(C)C)C2C] | 656.7/68% |
| tert-butyl 5-((5-(2,6- dimethoxy-4-(2-methyl-1- oxo-1,2-dihydro-2,7- naphthyridin-4-yl)benzyl)- 2,5-diazabicyclo[2.2.1] heptan-2-yl)methyl)-1- methyl-3,4-dihydroisoquinoline- 2(1H)-carboxylate | 1 | 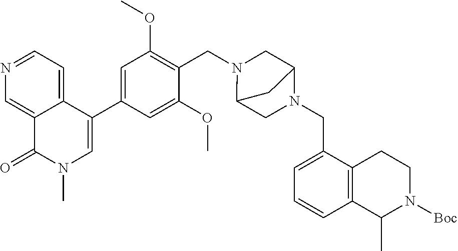 [SMILES: COc1cc(-c2cn(C)c(=O)c3cnccc23)cc(OC)c1CN1CC2CC1CN2Cc1cccc2c1CCN(C(=O)OC(C)(C)C)C2C] | 666.1/40% |
| tert-butyl 5-[[6-[[2,6- dimethoxy-4-(2-methyl-1- oxo-2,7-naphthyridin-4- yl)phenyl]methyl]-3,6- diazabicyclo[3.1.1]heptan-3- yl]methyl]-1-methyl-3,4- dihydro-1H-isoquinoline-2- carboxylate | 1 | 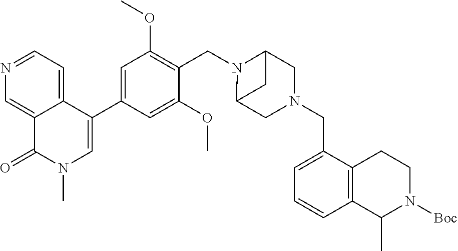 [SMILES: COc1cc(-c2cn(C)c(=O)c3cnccc23)cc(OC)c1CN1C2CC1CN(Cc1cccc3c1CCN(C(=O)OC(C)(C)C)C3C)C2] | 666.0/40% |
| tert-butyl 5-((3-(2,6- dimethoxy-4-(2-methyl-1- oxo-1,2- dihydro-2,7-naphthyridin-4- yl)benzyl)-3,6-diazabicyclo [3.1.1]heptan-6-yl)methyl)-1- methyl-3,4-dihydroisoquinoline- 2(1H)-carboxylate | 1 | 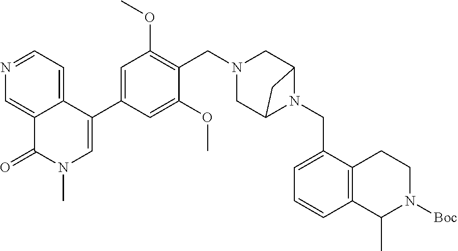 [SMILES: COc1cc(-c2cn(C)c(=O)c3cnccc23)cc(OC)c1CN1CC2CC(C1)N2Cc1cccc2c1CCN(C(=O)OC(C)(C)C)C2C] | 666.2/29% |
| tert-butyl 5-((3-(2,6- dimethoxy-4-(2-methyl-1- oxo-1,2-dihydro-2,7- naphthyridin-4-yl)benzyl)- 3,8-diazabicyclo[3.2.1]octan- 8-yl)methyl)-1-methyl- 3,4-dihydroisoquinoline- 2(1H)-carboxylate | 1 | 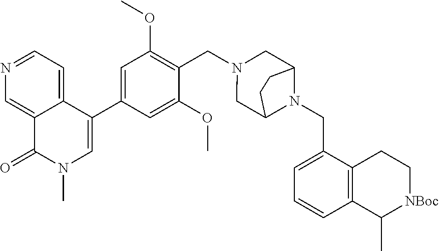 [SMILES: COc1cc(-c2cn(C)c(=O)c3cnccc23)cc(OC)c1CN1CC2CCC(C1)N2Cc1cccc2c1CCN(C(=O)OC(C)(C)C)C2C] | 680.5/36% |
| tert-butyl 5-((4-(4- (1,5-dimethyl-6-oxo-1,6- dihydropyridin-3-yl)-2,6- dimethoxybenzyl)piperazin- 1-yl)methyl)-1-methyl- 3,4-dihydroisoquinoline- 2(1H)-carboxylate (using aldehyde as prepared in WO2020/160198) | 1 | 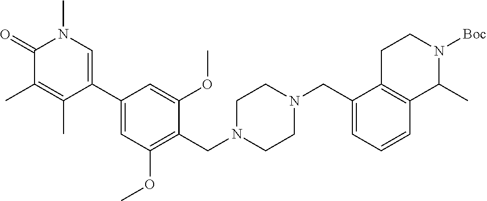 [SMILES: COc1cc(-c2cn(C)c(=O)c(C)c2C)cc(OC)c1CN1CCN(Cc2cccc3c2CCN(C(=O)OC(C)(C)C)C3C)CC1] | 617.5/89% |
| tert-butyl 5-((4- (2,6-dimethoxy-4-(1,4,5- trimethyl-6-oxo-1,6- dihydropyridin-3-yl)benzyl)- 1,4-diazepan-1-yl)methyl)-1- methyl-3,4-dihydroisoquinoline- 2(1H)-carboxylate | 1 | 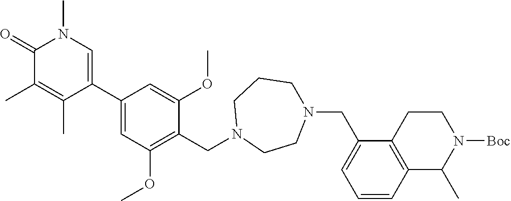 [SMILES: COc1cc(-c2cn(C)c(=O)c(C)c2C)cc(OC)c1CN1CCCN(Cc2cccc3c2CCN(C(=O)OC(C)(C)C)C3C)CC1] | 645.3/60% |
| tert-butyl 5-((5-(2,6- dimethoxy-4-(1,4,5-trimethyl-6-oxo- 1,6-dihydropyridin-3-yl)benzyl)-2,5- diazabicyclo[2.2.1]heptan-2-yl)methyl)- 1-methyl-3,4-dihydroisoquinoline- 2(1H)-carboxylate | 1 | 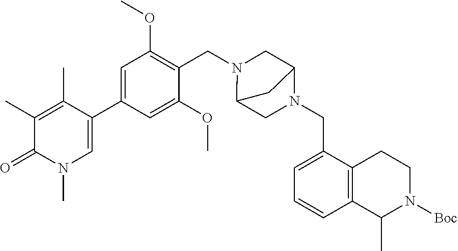 [SMILES: COc1cc(-c2cn(C)c(=O)c(C)c2C)cc(OC)c1CN1CC2CC1CN2Cc1cccc2c1CCN(C(=O)OC(C)(C)C)C2C] | 643.4/47% |
| tert-butyl 5-((3- (2,6-dimethoxy-4-(1,4,5-trimethyl- 6-oxo-1,6-dihydropyridin-3-yl)benzyl)- 3,6-diazabicyclo[3.1.1]heptan-6- yl)methyl)-1-methyl-3,4- dihydroisoquinoline-2(1H)-carboxylate | 1 | 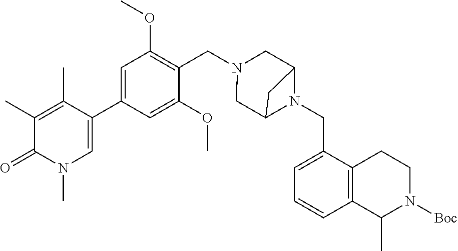 [SMILES: COc1cc(-c2cn(C)c(=O)c(C)c2C)cc(OC)c1CN1CC2CC(C1)N2Cc1cccc2c1CCN(C(=O)OC(C)(C)C)C2C] | 643.7/55% |
| tert-butyl 5-((4-(2,6- dimethoxy-4-(6-methyl-7-oxo-6,7- dihydro-1H-pyrazolo[3,4-c]pyridin-4- yl)benzyl)-4,7-diazaspiro[2.5]octan-7- yl)methyl)-1-methyl-3,4- dihydroisoquinoline-2(1H)- carboxylate | 1 | 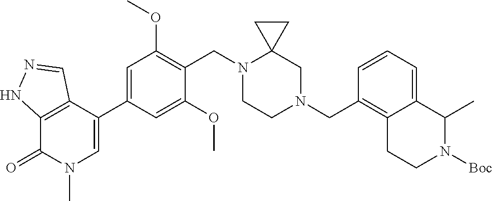 [SMILES: COc1cc(-c2cn(C)c(=O)c3[nH]ncc23)cc(OC)c1CN1CCN(Cc2cccc3c2CCN(C(=O)OC(C)(C)C)C3C)CC12CC2] | 669.4/67% |
| tert-butyl 5-((4-(2,6- dimethoxy-4-(1,4,5-trimethyl-6- oxo-1,6-dihydropyridin-3-yl) benzoyl)-1,4-diazepan-1-yl)methyl)- 1-methyl-3,4- dihydroisoquinoline-2(1H)- carboxylate | 1 | 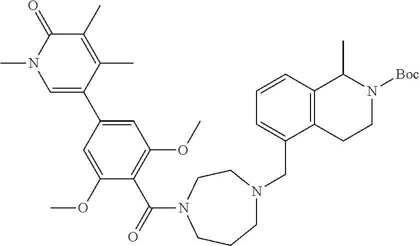 [SMILES: COc1cc(-c2cn(C)c(=O)c(C)c2C)cc(OC)c1C(=O)N1CCCN(Cc2cccc3c2CCN(C(=O)OC(C)(C)C)C3C)CC1] | 695.3/64% |
| tert-butyl 5-((7-(2,6- dimethoxy-4-(1,4,5-trimethyl-6-oxo- 1,6-dihydropyridin-3-yl)benzyl)-4,7- diazaspiro[2.5]octan-4-yl)methyl)-1- methyl-3,4-dihydroisoquinoline- 2(1H)-carboxylate | 1 | 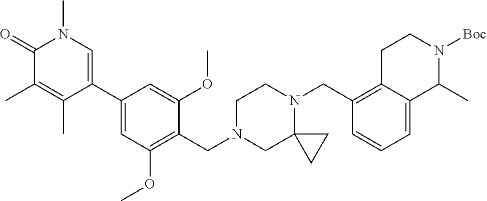 [SMILES: COc1cc(-c2cn(C)c(=O)c(C)c2C)cc(OC)c1CN1CCN(Cc2cccc3c2CCN(C(=O)OC(C)(C)C)C3C)C2(CC2)C1] | 657.3/82% |
| tert-butyl 5-(((2S,5S)- 4-(2,6-dimethoxy-4-(1,4,5- trimethyl-6-oxo-1,6-dihydropyridin- 3-yl)benzyl)-2,5-dimethylpiperazin-1- yl)methyl)-1-methyl-3,4- dihydroisoquinoline-2(1H)- carboxylate | 1 | 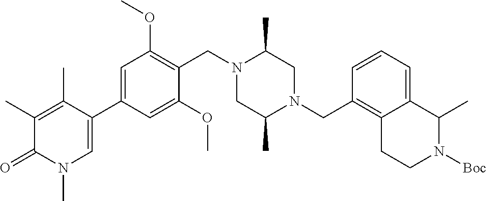 [SMILES: COc1cc(-c2cn(C)c(=O)c(C)c2C)cc(OC)c1CN1C[C@H](C)N(Cc2cccc3c2CCN(C(=O)OC(C)(C)C)C3C)C[C@@H]1C] | 659.4/52% |
| tert-butyl 5-(((2S,5R)- 4-(2,6-dimethoxy-4-(1,4,5- trimethyl-6-oxo-1,6-dihydropyridin- 3-yl)benzyl)-2,5-dimethylpiperazin- 1-yl)methyl)-1-methyl-3,4- dihydroisoquinoline-2(1H)- carboxylate | 1 | 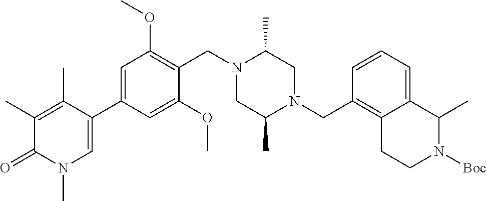 [SMILES: COc1cc(-c2cn(C)c(=O)c(C)c2C)cc(OC)c1CN1C[C@H](C)N(Cc2cccc3c2CCN(C(=O)OC(C)(C)C)C3C)C[C@H]1C] | 659.4/92% |
| tert-butyl 5-((4-(2,6- dimethoxy-4-(1,4,5-trimethyl-6- oxo-1,6-dihydropyridin-3-yl) benzoyl)piperazin-1-yl)methyl)-1- methyl-3,4- dihydroisoquinoline-2(1H)- carboxylate | 1 | 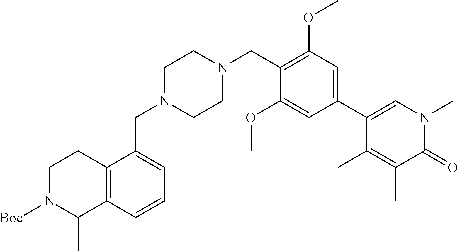 [SMILES: COc1cc(-c2cn(C)c(=O)c(C)c2C)cc(OC)c1CN1CCN(Cc2cccc3c2CCN(C(=O)OC(C)(C)C)C3C)CC1] | 645.3/82% |
| tert-butyl 5-((7-(2,6- dimethoxy-4-(6-methyl-7-oxo-6,7- dihydro-1H-pyrazolo[3,4-c]pyridin- 4-yl)benzyl)-4,7-diazaspiro[2.5]octan- 4-yl)methyl)-1-methyl-3,4- dihydroisoquinoline-2(1H)- carboxylate | 1 | 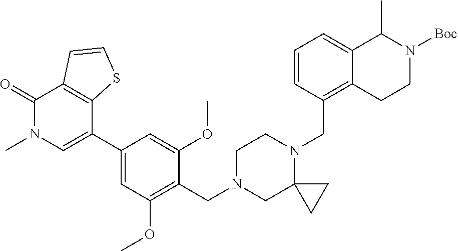 [SMILES: COc1cc(-c2cn(C)c(=O)c3ccsc23)cc(OC)c1CN1CCN(Cc2cccc3c2CCN(C(=O)OC(C)(C)C)C3C)C2(CC2)C1] | 669.4/53% |
| tert-butyl 5-((7-(2,6- dimethoxy-4-(5-methyl-4-oxo-4,5- dihydrothieno[3,2-c]pyridin-7- yl)benzyl)-4,7-diazaspiro[2.5]octan- 4-yl)methyl)-1-methyl-3,4- dihydroisoquinoline-2(1H)- carboxylate | 1 | 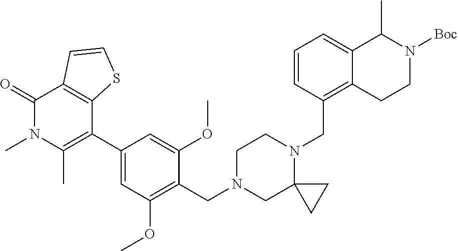 [SMILES: COc1cc(-c2c(C)n(C)c(=O)c3ccsc23)cc(OC)c1CN1CCN(Cc2cccc3c2CCN(C(=O)OC(C)(C)C)C3C)C2(CC2)C1] | 685.2/72% |
| tert-butyl 5-((3-(2,6- dimethoxy-4-(1,4,5-trimethyl-6- oxo-1,6-dihydropyridin-3-yl) benzyl)-3,8-diazabicyclo[3.2.1]octan- 8-yl)methyl)-1-methyl-3,4- dihydroisoquinoline-2(1H)- carboxylate | 1 | 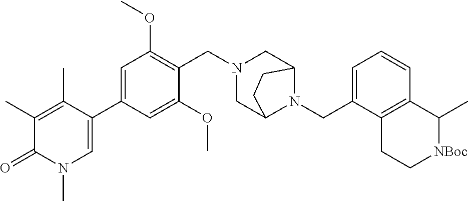 [SMILES: COc1cc(-c2cn(C)c(=O)c(C)c2C)cc(OC)c1CN1CC2CCC(C1)N2Cc1cccc2c1CCN(C(=O)OC(C)(C)C)C2C] | 657.3/75% |
| tert-butyl 5-((4-(2,6- dimethoxy-4-(6-methyl-7-oxo-6,7- dihydro-1H-pyrazolo[3,4-c]pyridin-4- yl)benzyl)piperazin-1-yl)methyl)-1- methyl-3,4- dihydroisoquinoline-2(1H)- carboxylate | 1 | 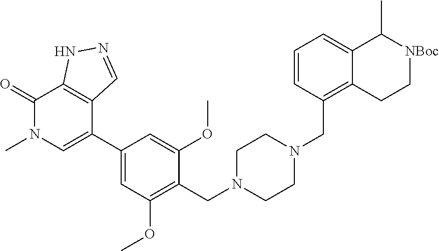 [SMILES: COc1cc(-c2cn(C)c(=O)c3[nH]ncc23)cc(OC)c1CN1CCN(Cc2cccc3c2CCN(C(=O)OC(C)(C)C)C3C)CC1] | 643.5/49% |
| tert-butyl 5-((4-(2,6- dimethoxy-4-(1,4,5-trimethyl-6-oxo- 1,6-dihydropyridin-3-yl)benzyl)- 2,2-dimethylpiperazin-1-yl) methyl)-1-methyl-3,4- dihydroisoquinoline-2(1H)- carboxylate | 1 | 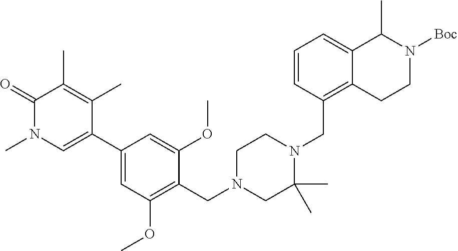 [SMILES: COc1cc(-c2cn(C)c(=O)c(C)c2C)cc(OC)c1CN1CCN(Cc2cccc3c2CCN(C(=O)OC(C)(C)C)C3C)C(C)(C)C1] | 659.4/41% |
| tert-butyl 5-(((2S,6S)-4-(2,6- dimethoxy-4-(1,4,5-trimethyl-6-oxo- 1,6-dihydropyridin-3-yl)benzyl)- 2,6-dimethylpiperazin-1-yl) methyl)-1-methyl-3,4- dihydroisoquinoline-2(1H)- carboxylate | 1 | 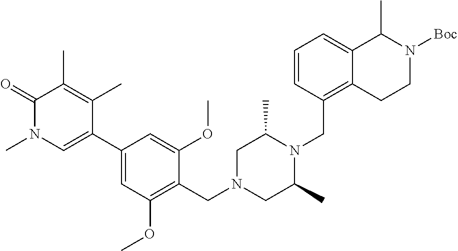 [SMILES: COc1cc(-c2cn(C)c(=O)c(C)c2C)cc(OC)c1CN1C[C@H](C)N(Cc2cccc3c2CCN(C(=O)OC(C)(C)C)C3C)[C@@H](C)C1] | 659.4/56% |
| tert-butyl 5-((5-(2,6-dimethoxy-4- (1,4,5-trimethyl-6-oxo-1,6- dihydropyridin-3-yl)benzyl)-2,5- diazabicyclo[2.2.2]octan-2-yl) methyl)-1-methyl-3,4- dihydroisoquinoline-2(1H)- carboxylate | 1 | 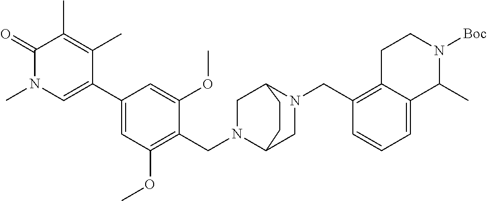 | 626.4/39% |
| tert-butyl 5-(((R)-4-(2,6-dimethoxy-4- (1,4,5-trimethyl-6-oxo-1,6- dihydropyridin-3-yl)benzyl)-2- methylpiperazin-1-yl)methyl)-1- methyl-3,4- dihydroisoquinoline-2(1H)- carboxylate | 1 | 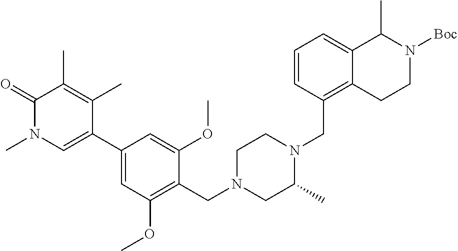 [SMILES: COc1cc(-c2cn(C)c(=O)c(C)c2C)cc(OC)c1CN1CCN(Cc2cccc3c2CCN(C(=O)OC(C)(C)C)C3C)[C@H](C)C1] | 654.3/92% |
| tert-butyl 5-(((S)-4-(2,6-dimethoxy- 4-(1,4,5-trimethyl-6-oxo-1,6- dihydropyridin-3-yl)benzyl)-2- methylpiperazin-1-yl)methyl)-1- methyl-3,4- dihydroisoquinoline-2(1H)- carboxylate | 1 | 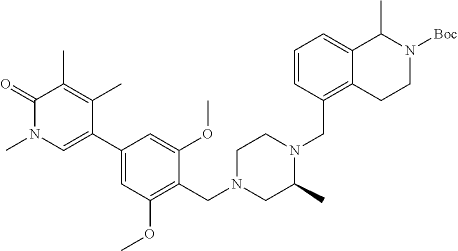 [SMILES: COc1cc(-c2cn(C)c(=O)c(C)c2C)cc(OC)c1CN1CCN(Cc2cccc3c2CCN(C(=O)OC(C)(C)C)C3C)[C@@H](C)C1] | 645.3/61% |
| tert-butyl 5-(((3S,5R)-4-(2,6- dimethoxy-4-(1,4,5-trimethyl-6- oxo-1,6-dihydropyridin-3-yl) benzyl)-3,5-dimethylpiperazin-1- yl)methyl)-1-methyl-3,4- dihydroisoquinoline-2(1H)- carboxylate | 1 | 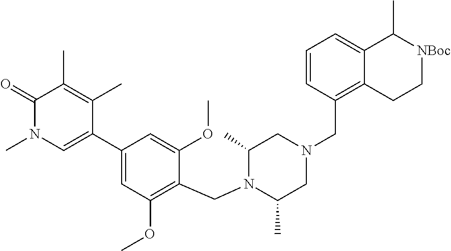 [SMILES: COc1cc(-c2cn(C)c(=O)c(C)c2C)cc(OC)c1CN1[C@H](C)CN(Cc2cccc3c2CCN(C(=O)OC(C)(C)C)C3C)C[C@@H]1C] | 659.4/36% |
| tert-butyl 5-((4-(2,6-dimethoxy-4- (1,4,5-trimethoxy-6-oxo-1,6- dihydropyridin-3-yl)benzyl)-4,7- diazaspiro[2.5]octan-7-yl)methyl)-1- methyl-3,4- dihydroisoquinoline-2(1H)- carboxylate | 1 | 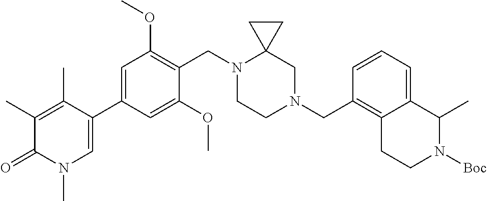 [SMILES: COc1cc(-c2cn(C)c(=O)c(C)c2C)cc(OC)c1CN1CCN(Cc2cccc3c2CCN(C(=O)OC(C)(C)C)C3C)CC12CC2] | 657.6/54% |
| tert-butyl 5-(((2R,5R)-4-(2,6- dimethoxy-4-(1,4,5-trimethyl-6- oxo-1,6-dihydropyridin-3-yl)benzyl)- 2,5-dimethylpiperazin-1-yl)methyl)- 1-methyl-3,4- dihydroisoquinoline-2(1H)- carboxylate | 1 | 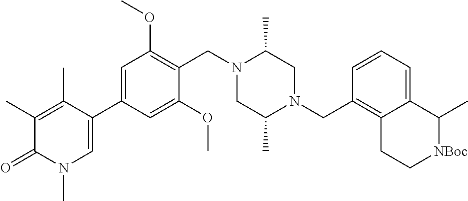 [SMILES: COc1cc(-c2cn(C)c(=O)c(C)c2C)cc(OC)c1CN1C[C@@H](C)N(Cc2cccc3c2CCN(C(=O)OC(C)(C)C)C3C)C[C@H]1C] | 659.4/21% |
| tert-butyl 7-(((2,6-dimethoxy-4- (1,4,5-trimethyl-6-oxo-1,6- dihydropyridin-3-yl)benzyl) (methyl)amino)methyl)-1-methyl- 3,4-dihydroisoquinoline-2(1H)- carboxylate | 3 | 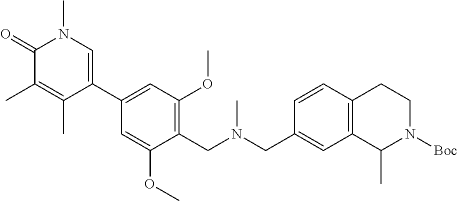 [SMILES: COc1cc(-c2cn(C)c(=O)c(C)c2C)cc(OC)c1CN(C)Cc1ccc2c(c1)C(C)N(C(=O)OC(C)(C)C)CC2] | 576.4/72% |
| tert-butyl 5-(((R)-4-(2,6-dimethoxy- 4-(1,4,5-trimethyl-6-oxo-1,6- dihydropyridin-3-yl)benzyl)-2- ethylpiperazin-1-yl)methyl)-1- methyl-3,4- dihydroisoquinoline-2(1H)- carboxylate | 1 | 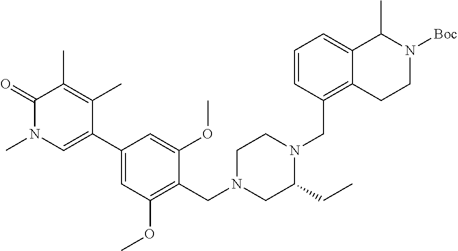 [SMILES: CC[C@@H]1CN(Cc2c(OC)cc(-c3cn(C)c(=O)c(C)c3C)cc2OC)CCN1Cc1cccc2c1CCN(C(=O)OC(C)(C)C)C2C] | 659.4/86% |
| tert-butyl 5-(((S)-4-(2,6-dimethoxy-4- (1,4,5-trimethyl-6-oxo-1,6- dihydropyridin-3-yl)benzyl)-2- ethylpiperazin-1-yl)methyl)-1- methyl-3,4- dihydroisoquinoline-2(1H)- carboxylate | 1 | 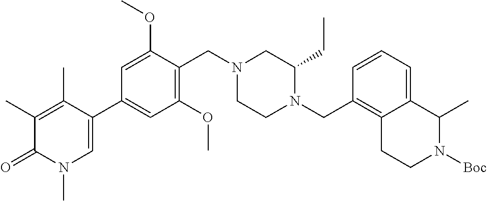 [SMILES: CC[C@H]1CN(Cc2c(OC)cc(-c3cn(C)c(=O)c(C)c3C)cc2OC)CCN1Cc1cccc2c1CCN(C(=O)OC(C)(C)C)C2C] | 659.4/41% |
| tert-butyl 5-(((2S,6S)-4-(2,6- dimethoxy-4-(1,4,5-trimethyl-6-oxo- 1,6-dihydropyridin-3-yl)benzyl)- 2,6-dimethylpiperazin-1-yl)methyl)- 1-methyl-3,4- dihydroisoquinoline-2(1H)- carboxylate | 1 | 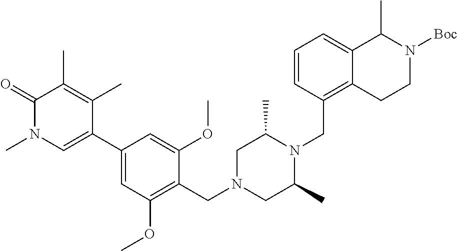 [SMILES: COc1cc(-c2cn(C)c(=O)c(C)c2C)cc(OC)c1CN1C[C@H](C)N(Cc2cccc3c2CCN(C(=O)OC(C)(C)C)C3C)[C@@H](C)C1] | 659.4/56% |
| tert-butyl 5-(((2,6-dimethoxy-4- (1,4,5-trimethyl-6-oxo-1,6- dihydropyridin-3-yl)benzyl)(methyl) amino)methyl)-1-methyl-3,4- dihydroisoquinoline-2(1H)- carboxylate | 1 | 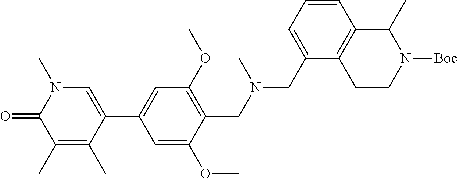 [SMILES: COc1cc(-c2cn(C)c(=O)c(C)c2C)cc(OC)c1CN(C)Cc1cccc2c1CCN(C(=O)OC(C)(C)C)C2C] | 576.4/73% |
| tert-butyl 5-(((S)-4-(2,6-dimethoxy-4- (1,4,5-trimethyl-6-oxo-1,6- dihydropyridin-3-yl)benzyl)-3- ethylpiperazin-1-yl)methyl)-1- methyl-3,4- dihydroisoquinoline-2(1H)- carboxylate | 1 | 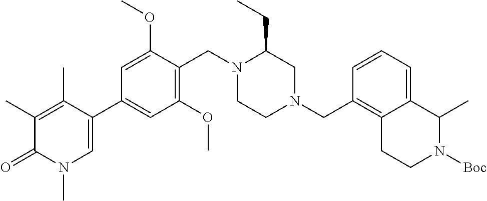 [SMILES: CC[C@H]1CN(Cc2cccc3c2CCN(C(=O)OC(C)(C)C)C3C)CCN1Cc1c(OC)cc(-c2cn(C)c(=O)c(C)c2C)cc1OC] | 659.4/55% |
| tert-butyl 5-((5-(2,6-dimethoxy-4- (1,4,5-trimethyl-6-oxo-1,6- dihydropyridin-3-yl)benzyl)-2,5- diazabicyclo[4.1.0]heptan-2- yl)methyl)-1-methyl-3,4- dihydroisoquinoline-2(1H)- carboxylate | 1 | 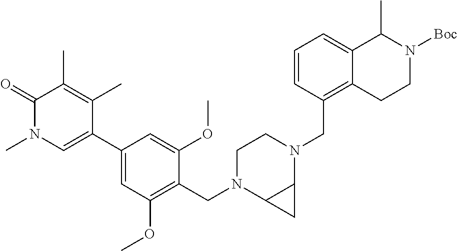 [SMILES: COc1cc(-c2cn(C)c(=O)c(C)c2C)cc(OC)c1CN1CCN(Cc2cccc3c2CCN(C(=O)OC(C)(C)C)C3C)C2CC21] | 643.4/16% |
| tert-butyl 5-(((2R,5S)-4-(2,6- dimethoxy-4-(1,4,5-trimethyl-6-oxo- 1,6-dihydropyridin-3-yl)benzyl)- 2,5-dimethylpiperazin-1-yl)methyl)- 1-methyl-3,4- dihydroisoquinoline-2(1H)- carboxylate | 1 | 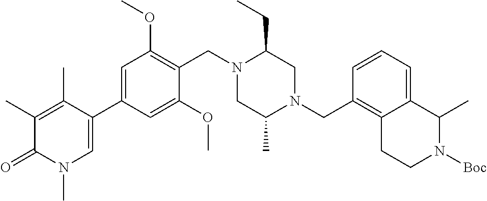 [SMILES: CC[C@H]1CN(Cc2cccc3c2CCN(C(=O)OC(C)(C)C)C3C)[C@H](C)CN1Cc1c(OC)cc(-c2cn(C)c(=O)c(C)c2C)cc1OC] | 659.4/75% |
| tert-butyl 5-((4-(2,6-dimethoxy-4- (1,4,5-trimethyl-6-oxo-1,6- dihydropyridin-3-yl)benzyl)-2- isopropylpiperazin-1-yl)methyl)- 1-methyl-3,4- dihydroisoquinoline-2(1H)- carboxylate | 1 | 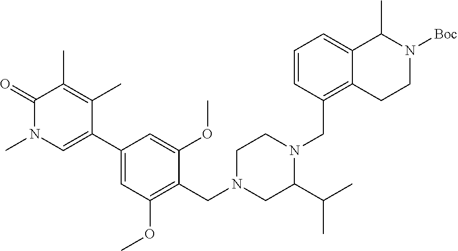 [SMILES: COc1cc(-c2cn(C)c(=O)c(C)c2C)cc(OC)c1CN1CCN(Cc2cccc3c2CCN(C(=O)OC(C)(C)C)C3C)C(C(C)C)C1] | 673.2/68% |
| tert-butyl 5-((8-(2,6-dimethoxy-4- (1,4,5-trimethyl-6-oxo-1,6- dihydropyridin-3-yl)benzyl)-5,8 diazaspiro[3.5]nonan-5-yl)methyl)-1- methyl-3,4- dihydroisoquinoline-2(1H)- carboxylate | 1 | 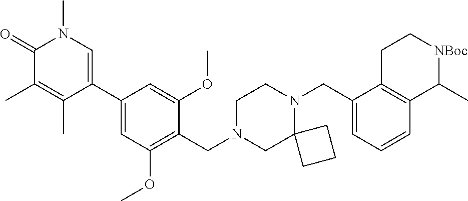 [SMILES: COc1cc(-c2cn(C)c(=O)c(C)c2C)cc(OC)c1CN1CCN(Cc2cccc3c2CCN(C(=O)OC(C)(C)C)C3C)C2(CCC2)C1] | 671.4/69% |
| tert-butyl 7-((4-(2,6-dimethoxy-4- (1,4,5-trimethyl-6-oxo-1,6- dihydropyridin-3-yl)benzyl)-4,7- diazaspiro[2.5]octan-7-yl)methyl)-1- methyl-3,4- dihydroisoquinoline-2(1H)- carboxylate | 3 | 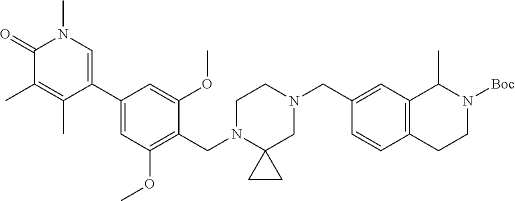 [SMILES: COc1cc(-c2cn(C)c(=O)c(C)c2C)cc(OC)c1CN1CCN(Cc2ccc3c(c2)C(C)N(C(=O)OC(C)(C)C)CC3)CC12CC2] | 657.3/67% |
| tert-butyl 7-((7-(2,6-dimethoxy-4- (1,4,5-trimethyl-6-oxo-1,6- dihydropyridin-3-yl)benzyl)-4,7- diazaspiro[2.5]octan-4-yl)methyl)-1- methyl-3,4- dihydroisoquinoline-2(1H)- carboxylate | 3 | 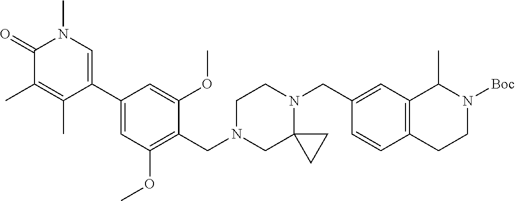 [SMILES: COc1cc(-c2cn(C)c(=O)c(C)c2C)cc(OC)c1CN1CCN(Cc2ccc3c(c2)C(C)N(C(=O)OC(C)(C)C)CC3)C2(CC2)C1] | 657.3/77% |
| tert-butyl 5-((7-(2,6-dimethoxy-4- (1,4,5-trimethyl-6-oxo-1,6- dihydropyridin-3-yl)benzoyl)-4,7- diazaspiro[2.5]octan-4-yl)methyl)-1- methyl-3,4- dihydroisoquinoline-2(1H)- carboxylate | 1 | 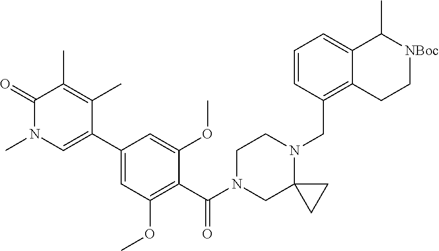 [SMILES: COc1cc(-c2cn(C)c(=O)c(C)c2C)cc(OC)c1C(=O)N1CCN(Cc2cccc3c2CCN(C(=O)OC(C)(C)C)C3C)C2(CC2)C1] | 671.6/40% |
| tert-butyl 5-((4-(2,6-dimethoxy-4-(5- methyl-4-oxo-4,5-dihydrothieno [3,2-c]pyridin-7-yl)benzyl) piperazin-1-yl)methyl)-1-methyl- 3,4-dihydroisoquinoline-2(1H)- carboxylate | 1 | 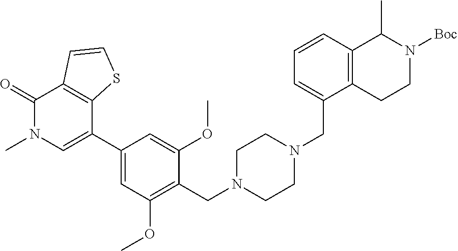 [SMILES: COc1cc(-c2cn(C)c(=O)c3ccsc23)cc(OC)c1CN1CCN(Cc2cccc3c2CCN(C(=O)OC(C)(C)C)C3C)CC1] | 659.5/32% |
| tert-butyl 5-(((R)-4-(2,6-dimethoxy- 4-(1,4,5-trimethyl-6-oxo-1,6- dihydropyridin-3-yl)benzyl)-3- ethylpiperazin-1-yl)methyl)-1- methyl-3,4- dihydroisoquinoline-2(1H)- carboxylate | 1 | 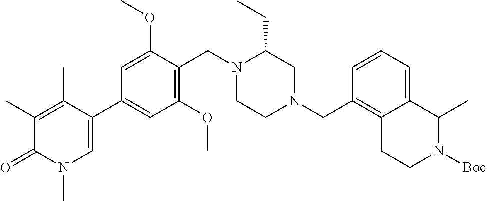 [SMILES: CC[C@@H]1CN(Cc2cccc3c2CCN(C(=O)OC(C)(C)C)C3C)CCN1Cc1c(OC)cc(-c2cn(C)c(=O)c(C)c2C)cc1OC] | 659.4/37% |
| tert-butyl 5-((6-(2,6-dimethoxy-4- (1,4,5-trimethyl-6-oxo-1,6- dihydropyridin-3-yl)benzyl)-3,6- diazabicyclo[3.1.1]heptan-3-yl)methyl)- 1-methyl-3,4- dihydroisoquinoline-2(1H)- carboxylate | 1 | 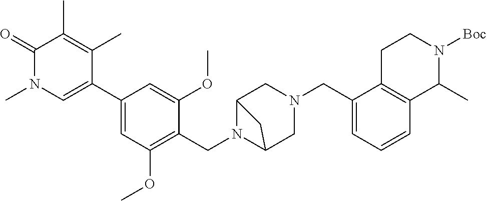 [SMILES: COc1cc(-c2cn(C)c(=O)c(C)c2C)cc(OC)c1CN1C2CC1CN(Cc1cccc3c1CCN(C(=O)OC(C)(C)C)C3C)C2] | 643.4/63% |
| tert-butyl 5-((4-(2,6-dimethoxy-4- (1,4,5-trimethyl-6-oxo-1,6- dihydropyridin-3-yl)benzyl)-3,3- dimethylpiperazin-1-yl)methyl)-1- methyl-3,4- dihydroisoquinoline-2(1H)- carboxylate | 1 | 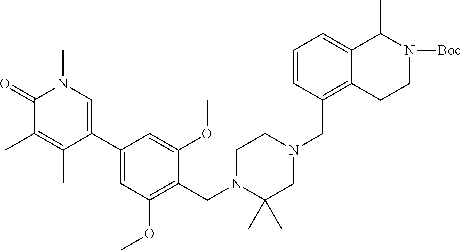 [SMILES: COc1cc(-c2cn(C)c(=O)c(C)c2C)cc(OC)c1CN1CCN(Cc2cccc3c2CCN(C(=O)OC(C)(C)C)C3C)CC1(C)C] | 659.3/71% |
| tert-butyl 7-((4-(2,6-dimethoxy-4- (1,4,5-trimethyl-6-oxo-1,6- dihydropyridin-3-yl)benzyl)-1,4- diazepan-1-yl)methyl)-1- methyl-3,4- dihydroisoquinoline-2(1H)- carboxylate | 3 | 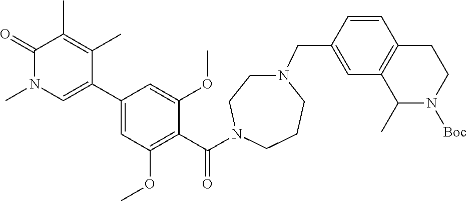 [SMILES: COc1cc(-c2cn(C)c(=O)c(C)c2C)cc(OC)c1C(=O)N1CCCN(Cc2ccc3c(c2)C(C)N(C(=O)OC(C)(C)C)CC3)CC1] | 645.3/20% |
| tert-butyl 7-((7-(2,6-dimethoxy-4- (1,4,5-trimethyl-6-oxo-1,6- dihydropyridin-3-yl)benzyl)-4,7- diazaspiro[2.5]octan-4-yl)methyl)- 3,4-dihydroisoquinoline-2(1H)- carboxylate | 4 | 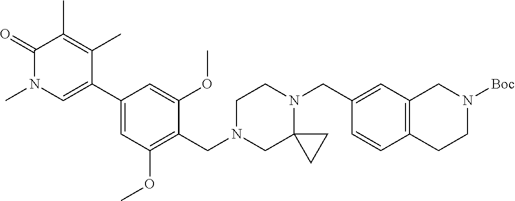 [SMILES: COc1cc(-c2cn(C)c(=O)c(C)c2C)cc(OC)c1CN1CCN(Cc2ccc3c(c2)CN(C(=O)OC(C)(C)C)CC3)C2(CC2)C1] | 643.4/24% |
| tert-butyl 5-((7-(2,6-dimethoxy-4- (1,4,5-trimethyl-6-oxo-1,6- dihydropyridin-3-yl)benzyl)-4,7- diazaspiro[2.5]octan-4-yl)methyl)- 3,4-dihydroisoquinoline-2(1H)- carboxylate | 5 | 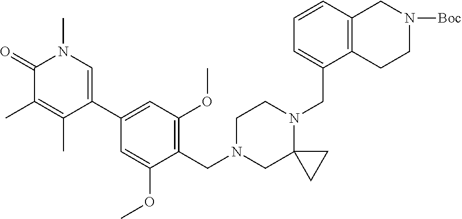 [SMILES: COc1cc(-c2cn(C)c(=O)c(C)c2C)cc(OC)c1CN1CCN(Cc2cccc3c2CCN(C(=O)OC(C)(C)C)C3)C2(CC2)C1] | 643.3/44% |
| Single diastereomer of tert-butyl 7- (((R)-4-(2,6-dimethoxy-4-(1,4,5- trimethyl-6-oxo-1,6-dihydropyridin- 3-yl)benzyl)-2-methylpiperazin-1- yl)methyl)-1-methyl-3,4- dihydroisoquinoline-2(1H)- carboxylate | 6 | 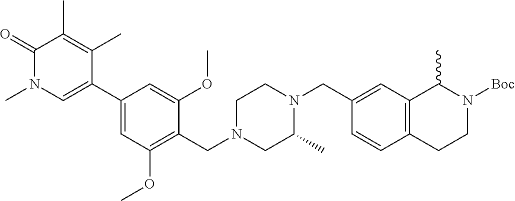 [SMILES: COc1cc(-c2cn(C)c(=O)c(C)c2C)cc(OC)c1CN1CCN(Cc2ccc3c(c2)C(C)N(C(=O)OC(C)(C)C)CC3)[C@H](C)C1] | 645.3/60% |
| tert-butyl 5-((7-(2,6-dimethoxy-4- (1,4,5-trimethyl-6-oxo-1,6- dihydropyridin-3-yl)benzyl)-4,7- diazaspiro[2.5]octan-4-yl)methyl)-1- methyl-3,4- dihydroisoquinoline-2(1H)- carboxylate | 7 | 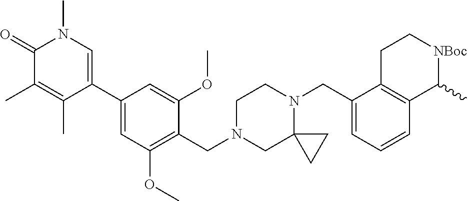 | 657.3/35% |
| tert-butyl 5-((7-(2,6-dimethoxy-4- (1,4,5-trimethyl-6-oxo-1,6- dihydropyridin-3-yl)benzyl)-4,7- diazaspiro[2.5]octan-4-yl)methyl)-1- methyl-3,4- dihydroisoquinoline-2(1H)- carboxylate | 7 | 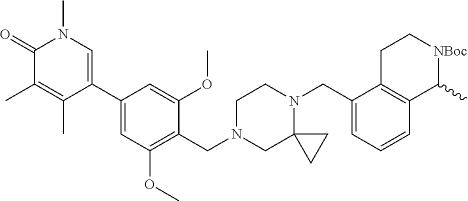 | 657.3/48% |
| tert-butyl 5-((4-(2,6-dimethoxy-4- (1,4,5-trimethyl-6-oxo-1,6- dihydropyridin-3-yl)benzyl)-1,4- diazepan-1-yl)methyl)-1- methyl-3,4- dihydroisoquinoline-2(1H)- carboxylate | 7 | 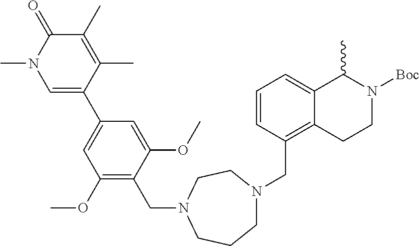 [SMILES: COc1cc(-c2cn(C)c(=O)c(C)c2C)cc(OC)c1CN1CCCN(Cc2cccc3c2CCN(C(=O)OC(C)(C)C)C3C)CC1] | 645.4/34% |
| tert-butyl 5-((4-(2,6-dimethoxy-4- (1,4,5-trimethyl-6-oxo-1,6- dihydropyridin-3-yl)benzyl)-4,7- diazaspiro[2.5]octan-7- yl)methyl)-1-methyl-3,4- dihydroisoquinoline-2(1H)- carboxylate | 7 | 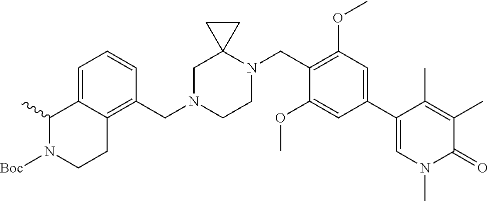 | 657.4/51% |
| tert-butyl 7-((7-(2-fluoro-6- methoxy-4-(1,4,5-trimethyl-6-oxo- 1,6-dihydropyridin-3-yl)benzyl)-4,7- diazaspiro[2.5]octan-4- yl)methyl)-1-methyl-3,4- dihydroisoquinoline-2(1H)- carboxylate | 6 | 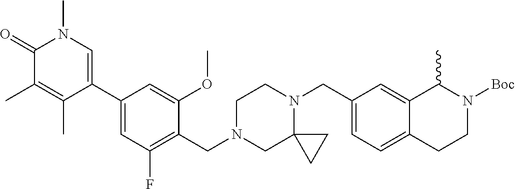 [SMILES: COc1cc(-c2cn(C)c(=O)c(C)c2C)cc(F)c1CN1CCN(Cc2ccc3c(c2)C(C)N(C(=O)OC(C)(C)C)CC3)C2(CC2)C1] | 645.4/60% |
| tert-butyl 7-((7-(2,6-dimethoxy-4- (6-methyl-7-oxo-6,7-dihydro-1H- pyrazolo[3,4-c]pyridin-4-yl)benzyl)- 4,7-diazaspiro[2.5]octan-4-yl)methyl)- 1-methyl-3,4- dihydroisoquinoline-2(1H)- carboxylate | 6 | 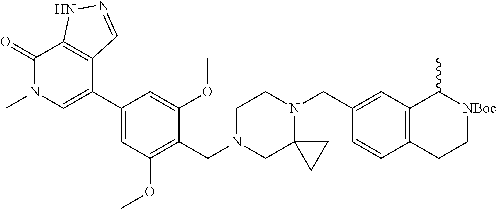 [SMILES: COc1cc(-c2cn(C)c(=O)c3[nH]ncc23)cc(OC)c1CN1CCN(Cc2ccc3c(c2)C(C)N(C(=O)OC(C)(C)C)CC3)C2(CC2)C1] | 669.3/68% |
| tert-butyl 5-((4-(2,6-dimethoxy-4- (1,4,5-trimethyl-6-oxo-1,6- dihydropyridin-3-yl)benzyl) piperazin-1-yl)methyl)-1- isopropyl-3,4- dihydroisoquinoline-2(1H)- carboxylate | 8 | 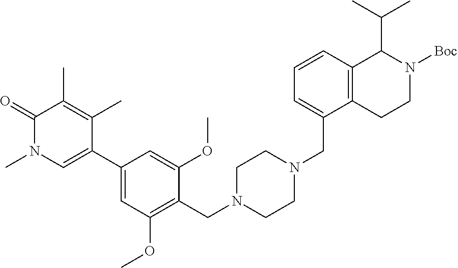 [SMILES: COc1cc(-c2cn(C)c(=O)c(C)c2C)cc(OC)c1CN1CCN(Cc2cccc3c2CCN(C(=O)OC(C)(C)C)C3C(C)C)CC1] | 659.7/30% |
| tert-butyl 5-((7-(2,6-dimethoxy-4- (1,4,5-trimethyl-6-oxo-1,6- dihydropyridin-3-yl)benzyl)-4,7- diazaspiro[2.5]octan-4-yl)methyl)- 1-isopropyl-3,4- dihydroisoquinoline-2(1H)- carboxylate | 8 | 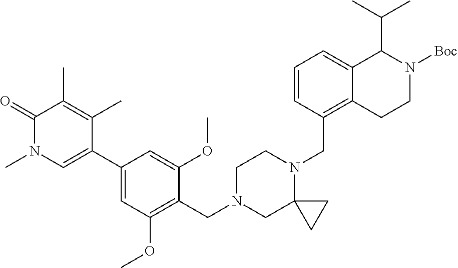 [SMILES: COc1cc(-c2cn(C)c(=O)c(C)c2C)cc(OC)c1CN1CCN(Cc2cccc3c2CCN(C(=O)OC(C)(C)C)C3C(C)C)C2(CC2)C1] | 684.3/49% |
| tert-butyl 5-((7-(2,6-dimethoxy-4- (1,4,5-trimethyl-6-oxo-1,6- dihydropyridin-3-yl)benzyl)-4,7- diazaspiro[2.5]octan-4-yl)methyl)- 1,1-dimethyl-3,4- dihydroisoquinoline-2(1H)- carboxylate | 9 | 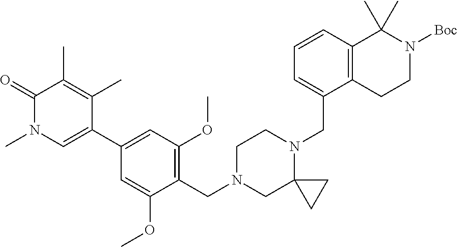 [SMILES: COc1cc(-c2cn(C)c(=O)c(C)c2C)cc(OC)c1CN1CCN(Cc2cccc3c2CCN(C(=O)OC(C)(C)C)C3(C)C)C2(CC2)C1] | 671.4/40% |
| tert-butyl 5-((4-(2,6-dimethoxy-4- (1,4,5-trimethyl-6-oxo-1,6- dihydropyridin-3-yl)benzyl)-4,7- diazaspiro[2.5]octan-7-yl)methyl)-1- isopropyl-3,4- dihydroisoquinoline-2(1H)- carboxylate | 8 | 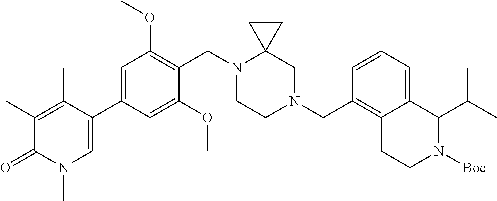 [SMILES: COc1cc(-c2cn(C)c(=O)c(C)c2C)cc(OC)c1CN1CCN(Cc2cccc3c2CCN(C(=O)OC(C)(C)C)C3C(C)C)CC12CC2] | 685.4/81% |
| tert-butyl 5-((4-(2,6-dimethoxy-4- (1,4,5-trimethyl-6-oxo-1,6- dihydropyridin--yl)benzyl)-1,4- diazepan-1-yl)methyl)-1-isopropyl- 3,4-dihydroisoquinoline-2(1H)- carboxylate | 8 | 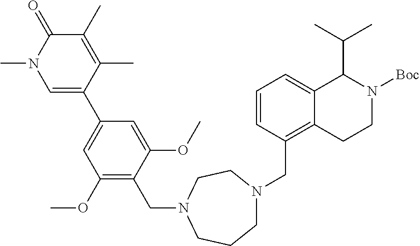 [SMILES: COc1cc(-c2cn(C)c(=O)c(C)c2C)cc(OC)c1CN1CCCN(Cc2cccc3c2CCN(C(=O)OC(C)(C)C)C3C(C)C)CC1] | 673.3/46% |
| tert-butyl 5-(((2R,5S)-4-(2,6- dimethoxy-4-(1,4,5-trimethyl-6- oxo-1,6-dihydropyridin-3-yl)benzyl)- 2,5-dimethylpiperazin-1-yl)methyl)- 1-isopropyl-3,4- dihydroisoquinoline-2(1H)- carboxylate | 8 | 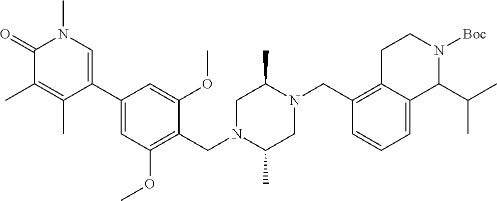 [SMILES: COc1cc(-c2cn(C)c(=O)c(C)c2C)cc(OC)c1CN1C[C@@H](C)N(Cc2cccc3c2CCN(C(=O)OC(C)(C)C)C3C(C)C)C[C@@H]1C] | 687.4/38% |
| tert-butyl 1-(difluoromethyl)-5-((7- (2,6-dimethoxy-4-(1,4,5-trimethyl- 6-oxo-1,6-dihydropyridin-3-yl) benzyl)-4,7-diazaspiro[2.5]octan-4- yl)methyl)-3,4- dihydroisoquinoline-2(1H)- carboxylate | 10 | 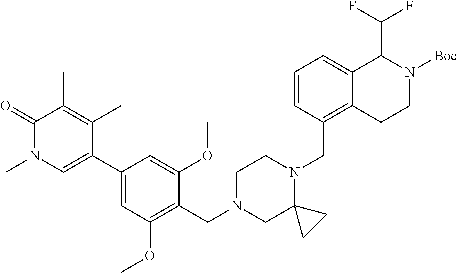 [SMILES: COc1cc(-c2cn(C)c(=O)c(C)c2C)cc(OC)c1CN1CCN(Cc2cccc3c2CCN(C(=O)OC(C)(C)C)C3C(F)F)C2(CC2)C1] | 693.4/69% |
| tert-butyl 5-(((S)-4-(2,6-dimethoxy- 4-(1,4,5-trimethyl-6-oxo-1,6- dihydropyridin-3-yl)benzyl)-2- methylpiperazin-1-yl)methyl)-1- isopropyl-3,4- dihydroisoquinoline-2(1H)- carboxylate | 8 | 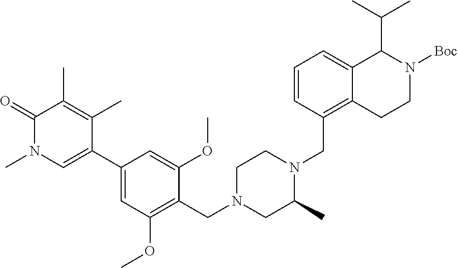 [SMILES: COc1cc(-c2cn(C)c(=O)c(C)c2C)cc(OC)c1CN1CCN(Cc2cccc3c2CCN(C(=O)OC(C)(C)C)C3C(C)C)[C@@H](C)C1] | 673.5/41% |
| tert-butyl 5-((7-(2,6-dimethoxy-4- (1,4,5-trimethyl-6-oxo-1,6- dihydropyridin-3-yl)benzyl)-4,7- diazaspiro[2.5]octan-4-yl)methyl)-1- ethyl-3,4- dihydroisoquinoline-2(1H)- carboxylate | 11 | 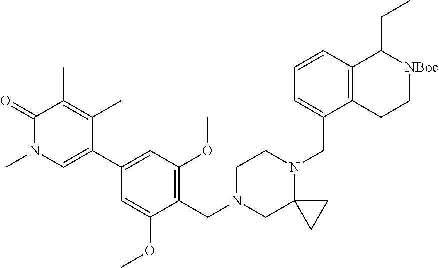 [SMILES: CCC1c2cccc(CN3CCN(Cc4c(OC)cc(-c5cn(C)c(=O)c(C)c5C)cc4OC)CC34CC4)c2CCN1C(=O)OC(C)(C)C] | 671.4/59% |
| tert-butyl 1-cyclopropyl-5-((7-(2,6- dimethoxy-4-(1,4,5-trimethyl-6-oxo- 1,6-dihydropyridin-3-yl)benzyl)-4,7- diazaspiro[2.5]octan-4-yl)methyl)- 3,4-dihydroisoquinoline-2(1H)- carboxylate | 12 | 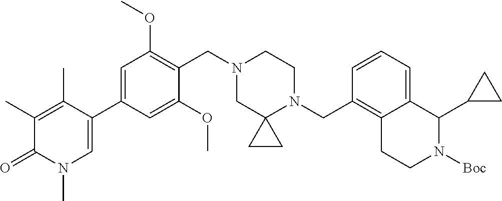 [SMILES: COc1cc(-c2cn(C)c(=O)c(C)c2C)cc(OC)c1CN1CCN(Cc2cccc3c2CCN(C(=O)OC(C)(C)C)C3C2CC2)C2(CC2)C1] | 683.3/47% |
| tert-butyl 5-((7-(2,6-dimethoxy-4- (1,4,5-trimethyl-6-oxo-1,6- dihydropyridin-3-yl)benzyl)-4,7- diazaspiro[2.5]octan-4-yl)methyl)-1- propyl-3,4- dihydroisoquinoline-2(1H)- carboxylate | 13 | 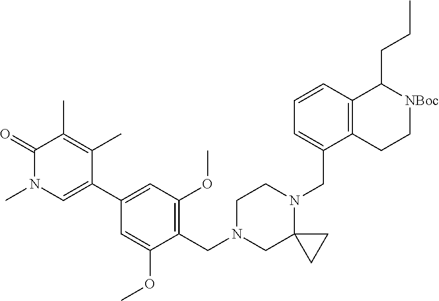 [SMILES: CCCC1c2cccc(CN3CCN(Cc4c(OC)cc(-c5cn(C)c(=O)c(C)c5C)cc4OC)CC34CC4)c2CCN1C(=O)OC(C)(C)C] | 685.9/59% |
| tert-butyl 7-((7-(2,6-dimethoxy-4- (1,4,5-trimethyl-6-oxo-1,6- dihydropyridin-3-yl)benzyl)-4,7- diazaspiro[2.5]octan-4-yl)methyl)-1- isopropyl-3,4- dihydroisoquinoline-2(1H)- carboxylate | 14 | 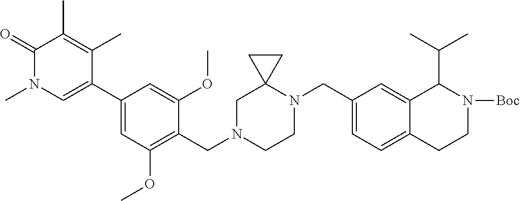 [SMILES: COc1cc(-c2cn(C)c(=O)c(C)c2C)cc(OC)c1CN1CCN(Cc2ccc3c(c2)C(C(C)C)N(C(=O)OC(C)(C)C)CC3)C2(CC2)C1] | 685.4/44% |
| tert-butyl 7-(((2,6-dimethoxy-4-(1,4,5- trimethyl-6-oxo-1,6-dihydropyridin-3- yl)benzyl)(methyl)amino)methyl)-1- isopropyl-3,4- dihydroisoquinoline-2(1H)- carboxylate | 14 | 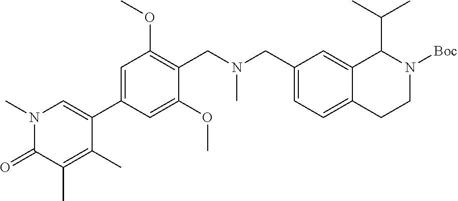 [SMILES: COc1cc(-c2cn(C)c(=O)c(C)c2C)cc(OC)c1CN(C)Cc1ccc2c(c1)C(C(C)C)N(C(=O)OC(C)(C)C)CC2] | 604.3/31% |
| tert-butyl 5-((7-(2,6-dimethoxy-4- (1,4,5-trimethyl-6-oxo-1,6- dihydropyridin-3-yl)benzyl)-4,7- diazaspiro[2.5]octan-4-yl)methyl)- 1-methoxy-1-(methoxymethyl)- 3,4-dihydroisoquinoline-2(1H)- carboxylate | 15 | 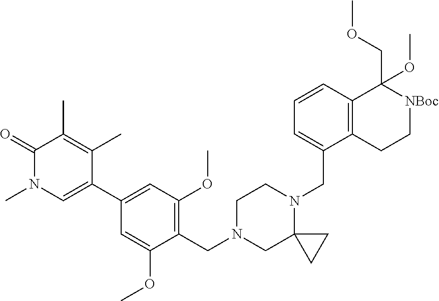 [SMILES: COCC1(OC)c2cccc(CN3CCN(Cc4c(OC)cc(-c5cn(C)c(=O)c(C)c5C)cc4OC)CC34CC4)c2CCN1C(=O)OC(C)(C)C] | 717.4/41% |
| tert-butyl 5-((4-(2,6-dimethoxy-4- (1,4,5-trimethyl-6-oxo-1,6- dihydropyridin-3-yl)benzyl)-4,7- diazaspiro[2.5]octan-7-yl)methyl)- 1-ethyl-3,4- dihydroisoquinoline-2(1H)-carboxylate | 16 | 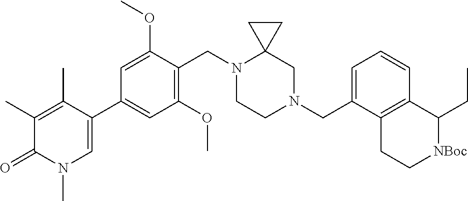 [SMILES: CCC1c2cccc(CN3CCN(Cc4c(OC)cc(-c5cn(C)c(=O)c(C)c5C)cc4OC)C4(CC4)C3)c2CCN1C(=O)OC(C)(C)C] | 671.3/74% |

Table 6a: The following examples were prepared by general procedure 2 using the starting materials synthesized in Table 5a.

TABLE 6a

| | | LCMS m/z |
|---|---|---|
| | | [M + H]⁺/ |
| Name | Product Structure | Yield |
| 4-(3,5-dimethoxy-4-((4-((1-methyl- 1,2,3,4-tetrahydroisoquinolin-5- yl)methyl)piperazin-1- yl)methyl)phenyl)-2-methyl-2,7- naphthyridin-1(2H)-one | 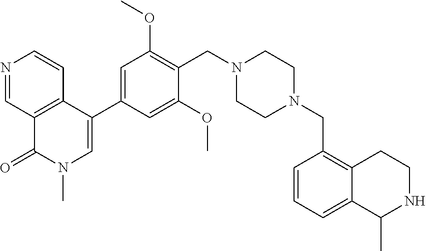 [SMILES: COc1cc(-c2cn(C)c(=O)c3cnccc23)cc(OC)c1CN1CCN(Cc2cccc3c2CCNC3C)CC1] | 554.2/95% |
| 5-(3,5-dimethoxy-4-((4-((1-methyl- 1,2,3,4-tetrahydroisoquinolin-5- yl)methyl)piperazin-1- yl)methyl)phenyl)-1,3,4- trimethylpyridin-2(1H)-one | 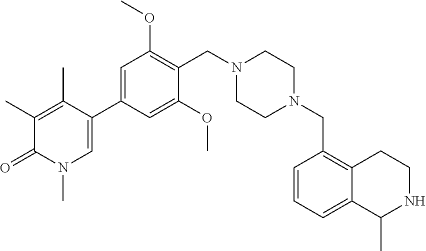 [SMILES: COc1cc(-c2cn(C)c(=O)c(C)c2C)cc(OC)c1CN1CCN(Cc2cccc3c2CCNC3C)CC1] | 531.0/98% |
| 4-(3,5-dimethoxy-4-((4-((1-methyl- 1,2,3,4-tetrahydroisoquinolin-5- yl)methyl)-1,4-diazepan-1- yl)methyl)phenyl)-2-methyl-2,7- naphthyridin-1(2H)-one | 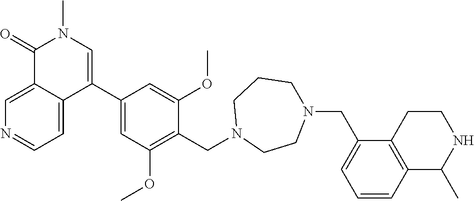 [SMILES: COc1cc(-c2cn(C)c(=O)c3cnccc23)cc(OC)c1CN1CCCN(Cc2cccc3c2CCNC3C)CC1] | 568.3/75% |
| 4-[3,5-dimethoxy-4-[[methyl-[(1- methyl-1,2,3,4-tetrahydroisoquinolin-5- yl)methyl]amino]methyl]phenyl]- 2-methyl-2,7-naphthyridin-1-one | 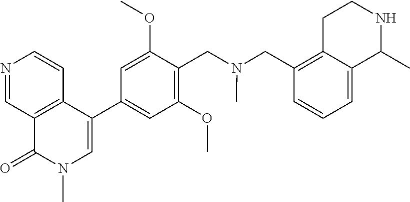 [SMILES: COc1cc(-c2cn(C)c(=O)c3cnccc23)cc(OC)c1CN(C)Cc1cccc2c1CCNC2C] | 499.0/97% |
| 4-[3,5-dimethoxy-4-[[methyl-[(1- methyl-1,2,3,4-tetrahydroisoquinolin- 6-yl)methyl]amino]methyl]phenyl]- 2-methyl-2,7-naphthyridin-1-one | 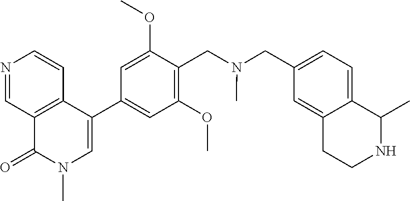 [SMILES: COc1cc(-c2cn(C)c(=O)c3cnccc23)cc(OC)c1CN(C)Cc1ccc2c(c1)CCNC2C] | 499.0/97% |
| 4-[3,5-dimethoxy-4-[[methyl-[(1- methyl-1,2,3,4-tetrahydroisoquinolin- 7-yl)methyl]amino]methyl]phenyl]- 2-methyl-2,7-naphthyridin-1-one | 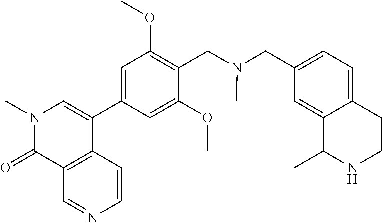 [SMILES: COc1cc(-c2cn(C)c(=O)c3cnccc23)cc(OC)c1CN(C)Cc1ccc2c(c1)C(C)NCC2] | 499.0/97% |
| 4-(3,5-dimethoxy-4-((methyl(2- (methyl((1-methyl- 1,2,3,4-tetrahydroisoquinolin-5-yl) methyl)amino)ethyl)amino)methyl) phenyl)-2-methyl-2,7- naphthyridin-1(2H)-one | 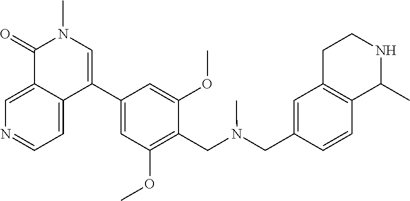 [SMILES: COc1cc(-c2cn(C)c(=O)c3cnccc23)cc(OC)c1CN(C)Cc1ccc2c(c1)CCNC2C] | 556.4/84% |
| 4-(3,5-dimethoxy-4-((5-((1-methyl- 1,2,3,4-tetrahydroisoquinolin-5- yl)methyl)-2,5- diazabicyclo[2.2.1]heptan-2- yl)methyl)phenyl)-2-methyl-2,7- naphthyridin-1(2H)-one | 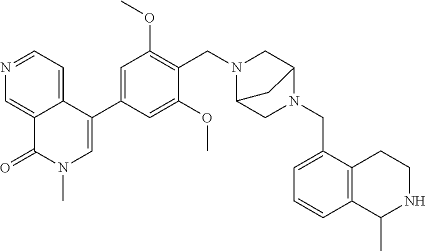 [SMILES: COc1cc(-c2cn(C)c(=O)c3cnccc23)cc(OC)c1CN1CC2CC1CN2Cc1cccc2c1CCNC2C] | 566.1/98% |
| 4-[3,5-dimethoxy-4-[[3-[(1-methyl- 1,2,3,4-tetrahydroisoquinolin-5- yl)methyl]-3,6- diazabicyclo[3.1.1]heptan-6- yl]methyl]phenyl]-2-methyl-2,7- naphthyridin-1-one | 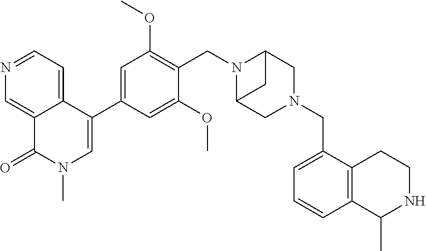 [SMILES: COc1cc(-c2cn(C)c(=O)c3cnccc23)cc(OC)c1CN1C2CC1CN(Cc1cccc3c1CCNC3C)C2] | 566.1/95% |
| 4-(3,5-dimethoxy-4-((6-((1-methyl- 1,2,3,4-tetrahydroisoquinolin-5-yl)- methyl)-3,6-diazabicyclo[3.1.1] heptan-3-yl)methyl)phenyl)-2- methyl-2,7-naphthyridin-1(2H)-one | 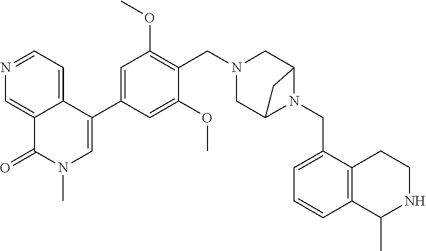 [SMILES: COc1cc(-c2cn(C)c(=O)c3cnccc23)cc(OC)c1CN1CC2CC(C1)N2Cc1cccc2c1CCNC2C] | 566.1/96% |
| 4-(3,5-dimethoxy-4-((8-((1-methyl- 1,2,3,4-tetrahydroisoquinolin-5- yl)methyl)-3,8- diazabicyclo[3.2.1]octan-3- yl)methyl)phenyl)-2-methyl-2,7- naphthyridin-1(2H)-one | 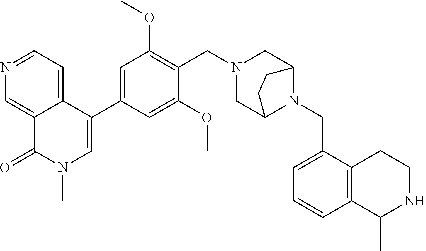 [SMILES: COc1cc(-c2cn(C)c(=O)c3cnccc23)cc(OC)c1CN1CC2CCC(C1)N2Cc1cccc2c1CCNC2C] | 579.3/98% |
| 5-(3,5-dimethoxy-4-((4-((1-methyl- 1,2,3,4-tetrahydroisoquinolin-5- yl)methyl)piperazin-1- yl)methyl)phenyl)-1,3- dimethylpyridin-2(1H)-one | 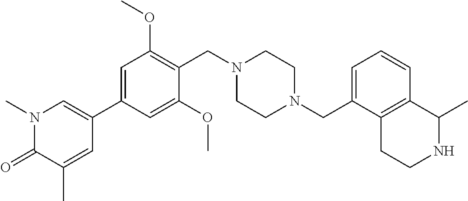 | 517.5/86% |
| 5-(3,5-dimethoxy-4-((4-((1-methyl- 1,2,3,4-tetrahydroisoquinolin-5- yl)methyl)-1,4-diazepan-1- yl)methyl)phenyl)-1,3,4- trimethylpyridin-2(1H)-one | 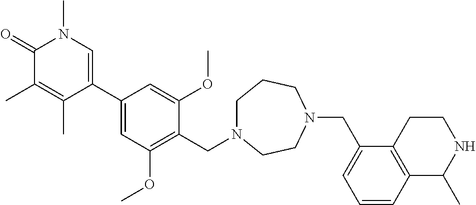 [SMILES: COc1cc(-c2cn(C)c(=O)c(C)c2C)cc(OC)c1CN1CCCN(Cc2cccc3c2CCNC3C)CC1] | 545.6/67% |
| 5-(3,5-dimethoxy-4-((5-((1-methyl- 1,2,3,4-tetrahydroisoquinolin-5- yl)methyl)-2,5- diazabicyclo[2.2.1]heptan-2- yl)methyl)phenyl)-1,3,4- trimethylpyridin-2(1H)-one | 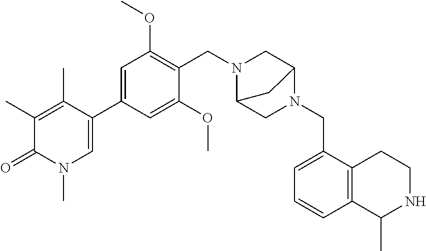 [SMILES: COc1cc(-c2cn(C)c(=O)c(C)c2C)cc(OC)c1CN1CC2CC1CN2Cc1cccc2c1CCNC2C] | 543.4/99% |
| 5-(3,5-dimethoxy-4-((6-((1-methyl- 1,2,3,4-tetrahydroisoquinolin-5- yl)methyl)-3,6- diazabicyclo[3.1.1]heptan-3- yl)methyl)phenyl)-1,3,4- trimethylpyridin-2(1H)-one | 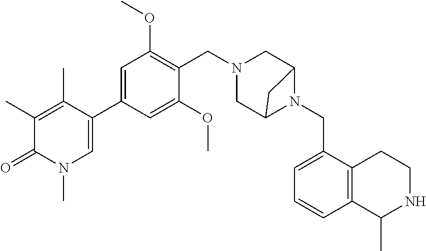 [SMILES: COc1cc(-c2cn(C)c(=O)c(C)c2C)cc(OC)c1CN1CC2CC(C1)N2Cc1cccc2c1CCNC2C] | 543.7/63% |
| 4-(3,5-dimethoxy-4-((7-((1-methyl- 1,2,3,4-tetrahydroisoquinolin-5- yl)methyl)-4,7-diazaspiro[2.5]octan-4- yl)methyl)phenyl)-6-methyl-1,6- dihydro-7H-pyrazolo[3,4-c]pyridin-7- one | 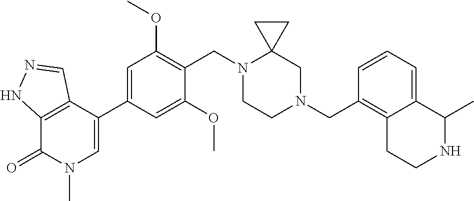 [SMILES: COc1cc(-c2cn(C)c(=O)c3[nH]ncc23)cc(OC)c1CN1CCN(Cc2cccc3c2CCNC3C)CC12CC2] | 569.2/95% |
| 5-(3,5-dimethoxy-4-(4-((1-methyl- 1,2,3,4-tetrahydroisoquinolin-5- yl)methyl)-1,4-diazepane-1- carbonyl)phenyl)-1,3,4- trimethylpyridin-2(1H)-one | 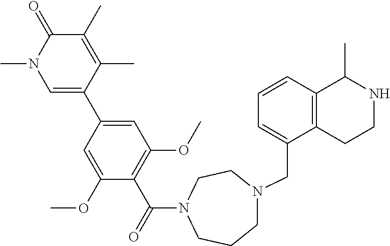 [SMILES: COc1cc(-c2cn(C)c(=O)c(C)c2C)cc(OC)c1C(=O)N1CCCN(Cc2cccc3c2CCNC3C)CC1] | 559.6/83% |
| 5-(3,5-dimethoxy-4-((4-((1-methyl- 1,2,3,4-tetrahydroisoquinolin-5- yl)methyl)-4,7-diazaspiro[2.5]octan-7- yl)methyl)phenyl)-1,3,4- trimethylpyridin-2(1H)-one | 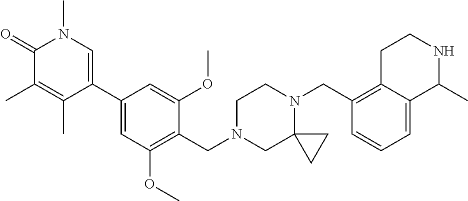 [SMILES: COc1cc(-c2cn(C)c(=O)c(C)c2C)cc(OC)c1CN1CCN(Cc2cccc3c2CCNC3C)C2(CC2)C1] | 557.3/91% |
| 5-(4-(((2S,5S)-2,5-dimethyl-4-((1- methyl-1,2,3,4-tetrahydroisoquinolin- 5-yl)methyl)piperazin-1-yl)methyl)- 3,5-dimethoxyphenyl)-1,3,4- trimethylpyridin-2(1H)-one | 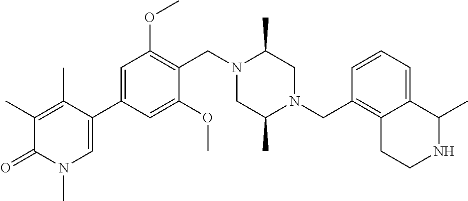 [SMILES: COc1cc(-c2cn(C)c(=O)c(C)c2C)cc(OC)c1CN1C[C@H](C)N(Cc2cccc3c2CCNC3C)C[C@@H]1C] | 559.3/93% |
| 5-(4-(((2R,5S)-2,5-dimethyl-4-((1- methyl-1,2,3,4-tetrahydroisoquinolin- 5-yl)methyl)piperazin-1-yl)methyl)- 3,5-dimethoxyphenyl)-1,3,4- trimethylpyridin-2(1H)-one | 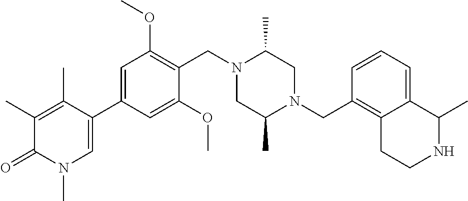 [SMILES: COc1cc(-c2cn(C)c(=O)c(C)c2C)cc(OC)c1CN1C[C@H](C)N(Cc2cccc3c2CCNC3C)C[C@H]1C] | 559.3/87% |
| 5-(3,5-dimethoxy-4-(4-((1-methyl- 1,2,3,4-tetrahydroisoquinolin-5- yl)methyl)piperazine-1- carbonyl)phenyl)-1,3,4- trimethylpyridin-2(1H)-one | 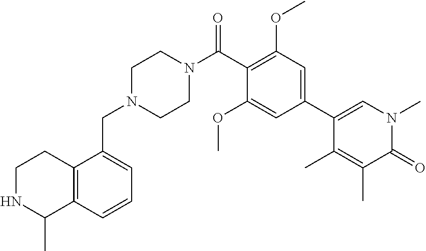 [SMILES: COc1cc(-c2cn(C)c(=O)c(C)c2C)cc(OC)c1C(=O)N1CCN(Cc2cccc3c2CCNC3C)CC1] | 545.2/82% |
| 4-(3,5-dimethoxy-4-((4-((1-methyl- 1,2,3,4-tetrahydroisoquinolin-5- yl)methyl)-4,7-diazaspiro[2.5]octan- 7-yl)methyl)phenyl)-6-methyl-1,6- dihydro-7H-pyrazolo[3,4-c]pyridin- 7-one | 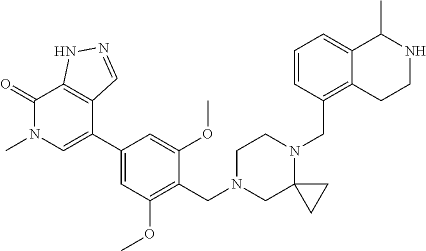 [SMILES: COc1cc(-c2cn(C)c(=O)c3[nH]ncc23)cc(OC)c1CN1CCN(Cc2cccc3c2CCNC3C)C2(CC2)C1] | 569.3/77% |
| 7-(3,5-dimethoxy-4-((4-((1-methyl- 1,2,3,4-tetrahydroisoquinolin-5- yl)methyl)-4,7-diazaspiro[2.5]octan-7- yl)methyl)phenyl)-5-methylthieno[3,2- c]pyridin-4(5H)-one | 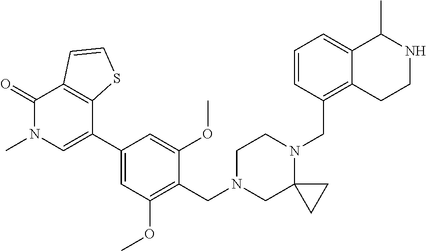 [SMILES: COc1cc(-c2cn(C)c(=O)c3ccsc23)cc(OC)c1CN1CCN(Cc2cccc3c2CCNC3C)C2(CC2)C1] | 585.3/82% |
| 5-(3,5-dimethoxy-4-((8-((1-methyl- 1,2,3,4-tetrahydroisoquinolin-5- yl)methyl)-3,8- diazabicyclo[3.2.1]octan-3- yl)methyl)phenyl)-1,3,4- trimethylpyridin-2(1H)-one | 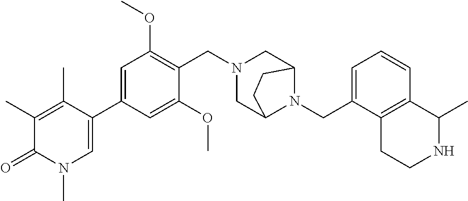 [SMILES: COc1cc(-c2cn(C)c(=O)c(C)c2C)cc(OC)c1CN1CC2CCC(C1)N2Cc1cccc2c1CCNC2C] | 557.3/87% |
| 5-(3,5-dimethoxy-4-((6-((1-methyl- 1,2,3,4-tetrahydroisoquinolin-5- yl)methyl)-3,6- diazabicyclo[3.1.1]heptan-3- yl)methyl)phenyl)-1,3,4- trimethylpyridin-2(1H)-one | 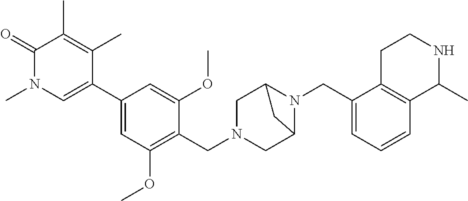 | 543.3/89% |
| 4-(3,5-dimethoxy-4-((4-((1-methyl- 1,2,3,4-tetrahydroisoquinolin-5- yl)methyl)piperazin-1- yl)methyl)phenyl)-6-methyl-1,6- dihydro-7H-pyrazolo[3,4-c]pyridin-7- one | 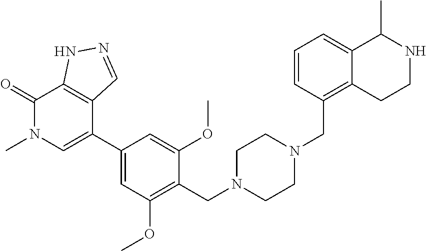 [SMILES: COc1cc(-c2cn(C)c(=O)c3[nH]ncc23)cc(OC)c1CN1CCN(Cc2cccc3c2CCNC3C)CC1] | 543.2/76% |
| 5-(4-((3,3-dimethyl-4-((1-methyl- 1,2,3,4-tetrahydroisoquinolin-5- yl)methyl)piperazin-1-yl)methyl)-3,5- dimethoxyphenyl)-1,3,4- trimethylpyridin-2(1H)-one | 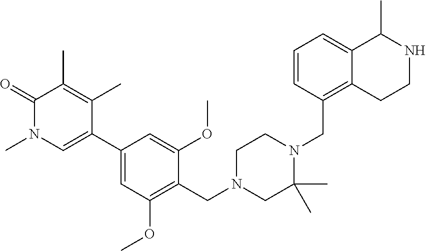 [SMILES: COc1cc(-c2cn(C)c(=O)c(C)c2C)cc(OC)c1CN1CCN(Cc2cccc3c2CCNC3C)C(C)(C)C1] | 559.2/89% |
| 5-(4-(((3S,5S)-3,5-dimethyl-4-((1- methyl-1,2,3,4-tetrahydroisoquinolin- 5-yl)methyl)piperazin-1-yl)methyl)- 3,5-dimethoxyphenyl)-1,3,4- trimethylpyridin-2(1H)-one | 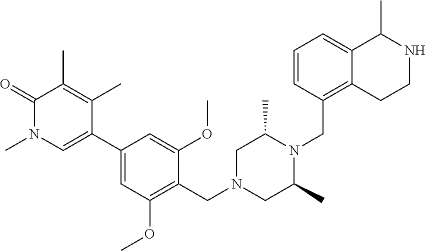 [SMILES: COc1cc(-c2cn(C)c(=O)c(C)c2C)cc(OC)c1CN1C[C@H](C)N(Cc2cccc3c2CCNC3C)[C@@H](C)C1] | 559.4/71% |
| 5-(3,5-dimethoxy-4-((5-((1-methyl- 1,2,3,4-tetrahydroisoquinolin-5- yl)methyl)-2,5- diazabicyclo[2.2.2]octan-2- yl)methyl)phenyl)-1,3,4- trimethylpyridin-2(1H)-one | 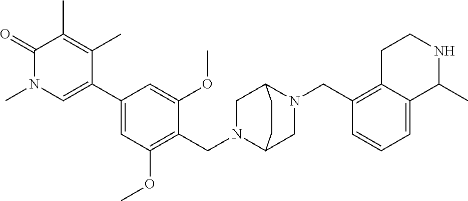 [SMILES: COc1cc(-c2cn(C)c(=O)c(C)c2C)cc(OC)c1CN1CC2CCC1CN2Cc1cccc2c1CCNC2C] | 557.3/88% |
| 5-(3,5-dimethoxy-4-(((3R)-3-methyl- 4-((1-methyl-1,2,3,4-tetrahydroisoquinolin-5- yl)methyl)piperazin-1-yl)methyl)phenyl)- 1,3,4-trimethylpyridin-2(1H)-one | 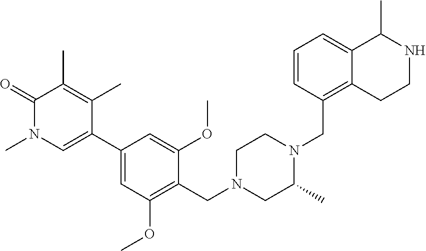 [SMILES: COc1cc(-c2cn(C)c(=O)c(C)c2C)cc(OC)c1CN1CCN(Cc2cccc3c2CCNC3C)[C@H](C)C1] | 545.3/81% |
| 5-(3,5-dimethoxy-4-(((3S)-3-methyl- 4-((1-methyl-1,2,3,4-tetrahydroisoquinolin- 5-yl)methyl)piperazin-1-yl)methyl) piperazin-1-yl)methyl)phenyl)-1,3,4- trimethylpyridin-2(1H)-one | 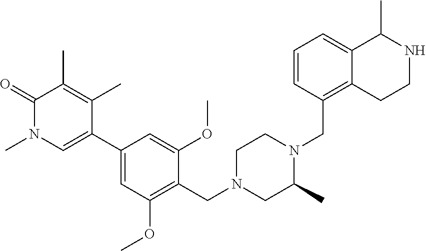 [SMILES: COc1cc(-c2cn(C)c(=O)c(C)c2C)cc(OC)c1CN1CCN(Cc2cccc3c2CCNC3C)[C@@H](C)C1] | 545.3/72% |
| 5-(4-(((2S,6R)-2,6-dimethyl-4-((1- methyl-1,2,3,4-tetrahydroisoquinolin- 5-yl)methyl)piperazin-1-yl)methyl)- 3,5-dimethoxyphenyl)-1,3,4- trimethylpyridin-2(1H)-one | 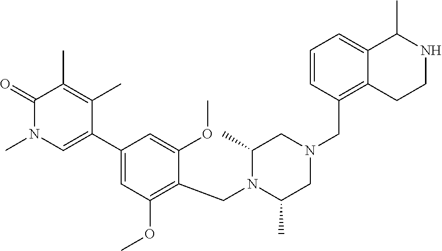 [SMILES: COc1cc(-c2cn(C)c(=O)c(C)c2C)cc(OC)c1CN1[C@H](C)CN(Cc2cccc3c2CCNC3C)C[C@@H]1C] | 559.4/82% |
| 5-(3,5-dimethoxy-4-((7-((1-methyl- 1,2,3,4-tetrahydroisoquinolin-5- yl)methyl)-4,7-diazaspiro[2.5]octan-4- yl)methyl)phenyl)-1,3,4- trimethylpyridin-2(1H)-one | 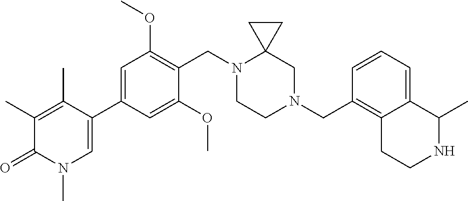 [SMILES: COc1cc(-c2cn(C)c(=O)c(C)c2C)cc(OC)c1CN1CCN(Cc2cccc3c2CCNC3C)CC12CC2] | 557.3/85% |
| 5-(4-(((2R,5R)-2,5-dimethyl-4-((1- methyl-1,2,3,4-tetrahydroisoquinolin- 5-yl)methyl)piperazin-1-yl)methyl)- 3,5-dimethoxyphenyl)-1,3,4- trimethylpyridin-2(1H)-one | 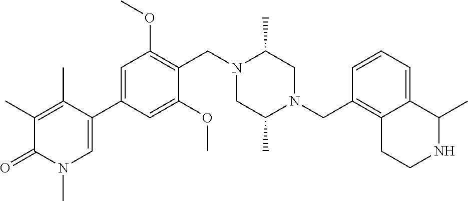 [SMILES: COc1cc(-c2cn(C)c(=O)c(C)c2C)cc(OC)c1CN1C[C@@H](C)N(Cc2cccc3c2CCNC3C)C[C@H]1C] | 559.4/87% |
| 5-(3,5-dimethoxy-4-((methyl((1- methyl-1,2,3,4-tetrahydroisoquinolin-7- yl)methyl)amino)methyl)phenyl)- 1,3,4-trimethylpyridin-2(1H)-one | 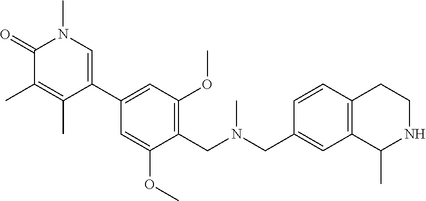 [SMILES: COc1cc(-c2cn(C)c(=O)c(C)c2C)cc(OC)c1CN(C)Cc1ccc2c(c1)C(C)NCC2] | 476.4/83% |
| 5-(4-(((3R)-3-ethyl-4-((1-methyl- 1,2,3,4-tetrahydroisoquinolin-5- yl)methyl)piperazin-1-yl)methyl)-3,5- dimethoxyphenyl)-1,3,4- trimethylpyridin-2(1H)-one | 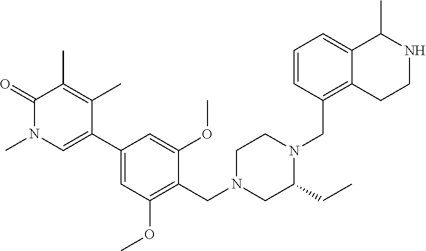 [SMILES: CC[C@@H]1CN(Cc2c(OC)cc(-c3cn(C)c(=O)c(C)c3C)cc2OC)CCN1Cc1cccc2c1CCNC2C] | 559.4/98% |
| 5-(4-(((3S)-3-ethyl-4-((1-methyl- 1,2,3,4-tetrahydroisoquinolin-5- yl)methyl)piperazin-1-yl)methyl)-3,5- dimethoxyphenyl)-1,3,4- trimethylpyridin-2(1H)-one | 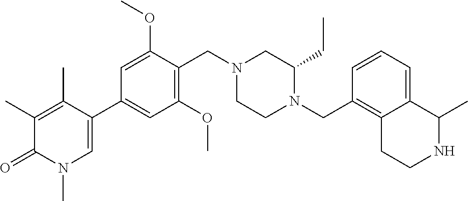 [SMILES: CC[C@H]1CN(Cc2c(OC)cc(-c3cn(C)c(=O)c(C)c3C)cc2OC)CCN1Cc1cccc2c1CCNC2C] | 559.4/91% |
| 5-(4-(((3S,5S)-3,5-dimethyl-4-((1- methyl-1,2,3,4-tetrahydroisoquinolin- 5-yl)methyl)piperazin-1-yl)methyl)- 3,5-dimethoxyphenyl)-1,3,4- trimethylpyridin-2(1H)-one | 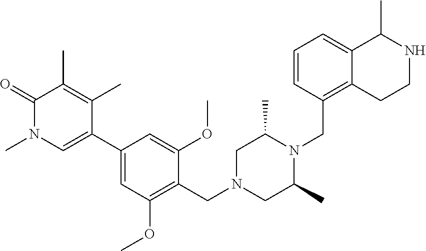 [SMILES: COc1cc(-c2cn(C)c(=O)c(C)c2C)cc(OC)c1CN1C[C@H](C)N(Cc2cccc3c2CCNC3C)[C@@H](C)C1] | 559.4/90% |
| 5-(3,5-dimethoxy-4-((methyl((1- methyl-1,2,3,4-tetrahydroisoquinolin- 5-yl)methyl)amino)methyl)phenyl)- 1,3,4-trimethylpyridin-2(1H)-one | 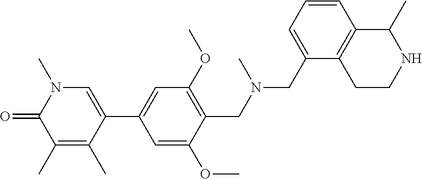 [SMILES: COc1cc(-c2cn(C)c(=O)c(C)c2C)cc(OC)c1CN(C)Cc1cccc2c1CCNC2C] | 476.4/89% |
| 5-(4-(((2S)-2-ethyl-4-((1-methyl- 1,2,3,4-tetrahydroisoquinolin-5- yl)methyl)piperazin-1-yl)methyl)-3,5- dimethoxyphenyl)-1,3,4- trimethylpyridin-2(1H)-one | 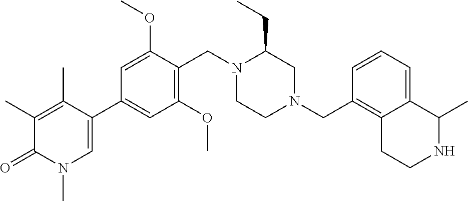 [SMILES: CC[C@H]1CN(Cc2cccc3c2CCNC3C)CCN1Cc1c(OC)cc(-c2cn(C)c(=O)c(C)c2C)cc1OC] | 559.2/78% |
| 5-(3,5-dimethoxy-4-((5-((1-methyl- 1,2,3,4-tetrahydroisoquinolin-5- yl)methyl)-2,5- diazabicyclo[4.1.0]heptan-2- yl)methyl)phenyl)-1,3,4- trimethylpyridin-2(1H)-one | 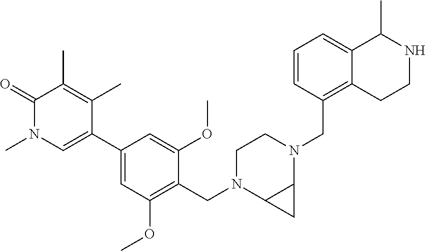 [SMILES: COc1cc(-c2cn(C)c(=O)c(C)c2C)cc(OC)c1CN1CCN(Cc2cccc3c2CCNC3C)C2CC21] | 543.2/79% |
| 5-(4-(((2S,5R)-2,5-dimethyl-4-((1- methyl-1,2,3,4-tetrahydroisoquinolin- 5-yl)methyl)piperazin-1-yl)methyl)- 3,5-dimethoxyphenyl)-1,3,4- trimethylpyridin-2(1H)-one | 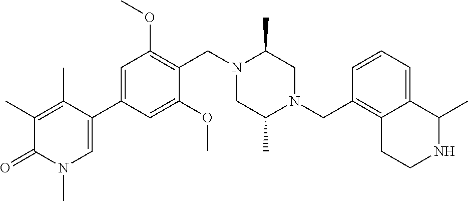 [SMILES: COc1cc(-c2cn(C)c(=O)c(C)c2C)cc(OC)c1CN1C[C@@H](C)N(Cc2cccc3c2CCNC3C)C[C@@H]1C] | 559.4/82% |
| 5-(4-((3-isopropyl-4-((1-methyl- 1,2,3,4-tetrahydroisoquinolin-5- yl)methyl)piperazin-1-yl)methyl-3,5- dimethoxyphenyl)-1,3,4- trimethylpyridin-2(1H)-one | 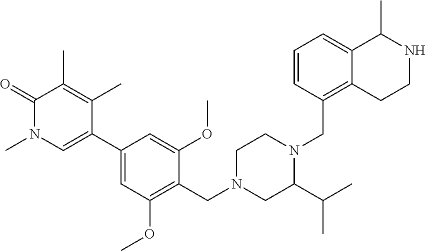 [SMILES: COc1cc(-c2cn(C)c(=O)c(C)c2C)cc(OC)c1CN1CCN(Cc2cccc3c2CCNC3C)C(C(C)C)C1] | 573.3/71% |
| 5-(3,5-dimethoxy-4-((5-((1-methyl- 1,2,3,4-tetrahydroisoquinolin-5- yl)methyl)-5,8-diazaspiro[3.5]nonan- 8-yl)methyl)phenyl)-1,3,4- trimethylpyridin-2(1H)-one | 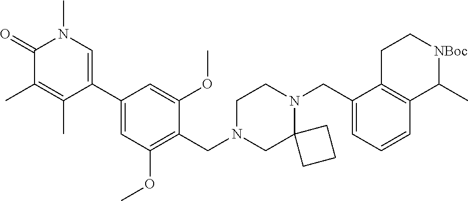 | 571.4/74% |
| 5-(3,5-dimethoxy-4-((4-((1-methyl- 1,2,3,4-tetrahydroisoquinolin-7- yl)methyl)-4,7-diazaspiro[2.5]octan-7- yl)methyl)phenyl)-1,3,4- trimethylpyridin-2(1H)-one | 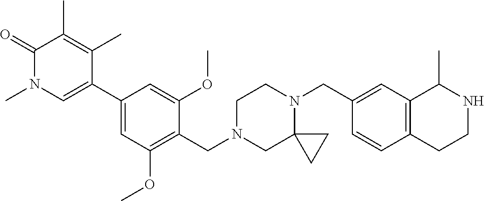 [SMILES: COc1cc(-c2cn(C)c(=O)c(C)c2C)cc(OC)c1CN1CCN(Cc2ccc3c(c2)C(C)NCC3)C2(CC2)C1] | 557.4/91% |
| 5-(3,5-dimethoxy-4-(4-((1-methyl- 1,2,3,4-tetrahydroisoquinolin-5- yl)methyl)-4,7-diazaspiro[2.5]octane- 7-carbonyl)phenyl)-1,3,4- trimethylpyridin-2(1H)-one | 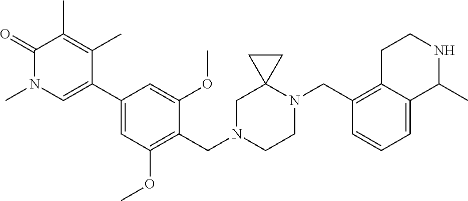 | 571.6/78% |
| 7-(3,5-dimethoxy-4-((4-((1-methyl- 1,2,3,4-tetrahydroisoquinolin-5- yl)methyl)piperazin-1- yl)methyl)phenyl)-5-methylthieno[3,2- c]pyridin-4(5H)-one | 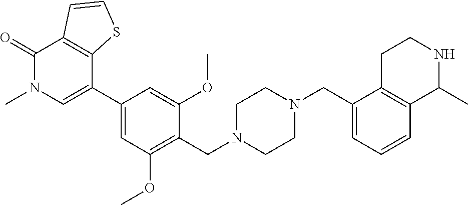 | 559.2/81% |
| 5-(4-(((2R)-2-ethyl-4-((1-methyl- 1,2,3,4-tetrahydroisoquinolin-5- yl)methyl)piperazin-1-yl)methyl)-3,5- dimethoxyphenyl)-1,3,4- trimethylpyridin-2(1H)-one | 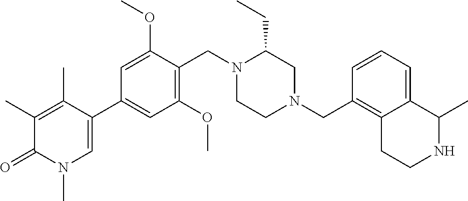 [SMILES: CC[C@@H]1CN(Cc2cccc3c2CCNC3C)CCN1Cc1c(OC)cc(-c2cn(C)c(=O)c(C)c2C)cc1OC] | 559.4/75% |
| 5-(3,5-dimethoxy-4-((3-((1-methyl- 1,2,3,4-tetrahydroisoquinolin-5- yl)methyl)-3,6- diazabicyclo[3.1.1]heptan-6- yl)methyl)phenyl)-1,3,4- trimethylpyridin-2(1H)-one | 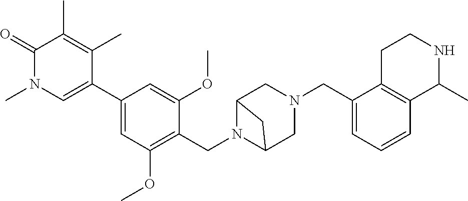 | 543.8/88% |
| 5-(4-((2,2-dimethyl-4-((1-methyl- 1,2,3,4-tetrahydroisoquinolin-5- yl)methyl)piperzin-1-yl)methyl)-3,5- dimethoxyphenyl)-1,3,4- trimethylpyridin-2(1H)-one | 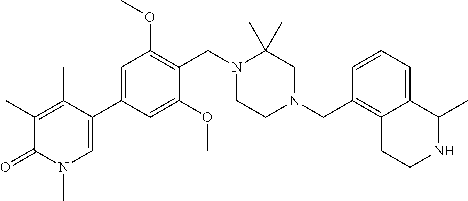 [SMILES: COc1cc(-c2cn(C)c(=O)c(C)c2C)cc(OC)c1CN1CCN(Cc2cccc3c2CCNC3C)CC1(C)C] | 559.2/91% |
| 5-(3,5-dimethoxy-4-((4- ((1,2,3,4-tetrahydroisoquinolin-7- yl)methyl)-4,7-diazaspiro[2.5]octan-7- yl)methyl)phenyl)-1,3,4- trimethylpyridin-2(1H)-one | 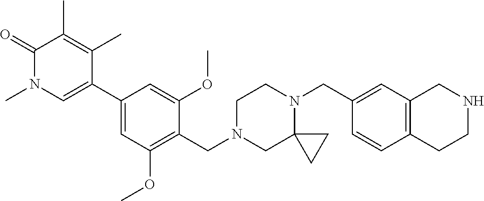 [SMILES: COc1cc(-c2cn(C)c(=O)c(C)c2C)cc(OC)c1CN1CCN(Cc2ccc3c(c2)CNCC3)C2(CC2)C1] | 557.4/95% |
| 5-(3,5-dimethoxy-4-((4- ((1,2,3,4-tetrahydroisoquinolin-5-yl)methyl)- 4,7-diazaspiro[2.5]octan-7-yl)methyl) phenyl)-1,3,4-trimethylpyridin- 2(1H)-one | 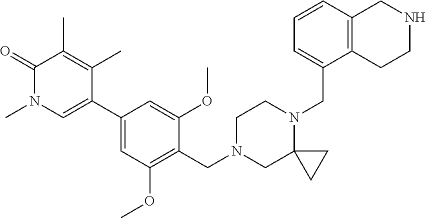 [SMILES: COc1cc(-c2cn(C)c(=O)c(C)c2C)cc(OC)c1CN1CCN(Cc2cccc3c2CCNC3)C2(CC2)C1] | 543.3/98% |
| Single diastereomer of 5-(3,5- dimethoxy-4-(((3R)-3-methyl-4-((1- methyl-1,2,3,4-tetrahydroisoquinolin- 7-yl)methyl)piperazin-1- yl)methyl)phenyl)-1,3,4- trimethylpyridin-2(1H)-one | single enantiomer of absolute unknown configuration 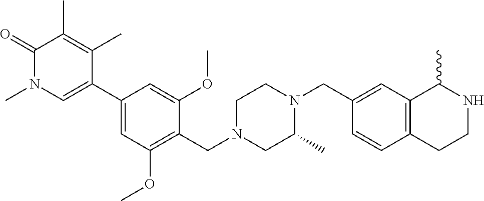 [SMILES: COc1cc(-c2cn(C)c(=O)c(C)c2C)cc(OC)c1CN1CCN(Cc2ccc3c(c2)C(C)NCC3)[C@H](C)C1] | 545.3/93% |
| 5-(3,5-dimethoxy-4-((4-((1-methyl- 1,2,3,4-tetrahydroisoquinolin-5- yl)methyl)-4,7-diazaspiro[2.5]octan-7- yl)methyl)phenyl)-1,3,4- trimethylpyridin-2(1H)-one | 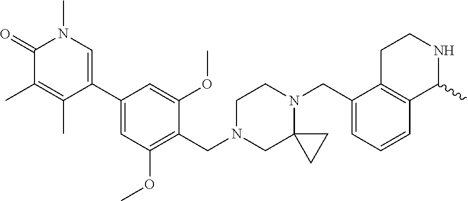 [SMILES: COc1cc(-c2cn(C)c(=O)c(C)c2C)cc(OC)c1CN1CCN(Cc2cccc3c2CCNC3C)C2(CC2)C1] | 557.2/84% |
| | single enantiomer of unknown absolute configuration | |
| | (using peak 1 of tert-butyl 5-bromo-1-methyl-3,4- | |
| | dihydroisoquinoline-2(1H)-carboxylate) | |
| 5-(3,5-dimethoxy-4-((4-((1-methyl- 1,2,3,4-tetrahydroisoquinolin-5- yl)methyl)-4,7-diazaspiro[2.5]octan-7- yl)methyl)phenyl)-1,3,4- trimethylpyridin-2(1H)-one | 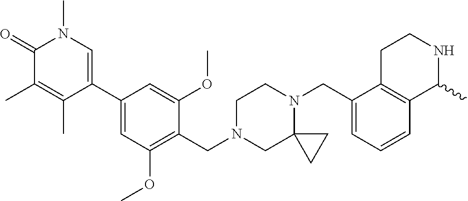 [SMILES: COc1cc(-c2cn(C)c(=O)c(C)c2C)cc(OC)c1CN1CCN(Cc2cccc3c2CCNC3C)C2(CC2)C1] | 557.2/89% |
| | single enantiomer of unknown absolute configuration | |
| | (using peak 2 of tert-butyl 5-bromo-1-methyl-3,4- | |
| | dihydroisoquinoline-2(1H)-carboxylate) | |
| 5-(3,5-dimethoxy-4-((4-((1-methyl 1,2,3,4-tetrahydroisoquinolin-5- yl)methyl)-1,4-diazepan-1- yl)methyl)phenyl)-1,3,4- trimethylpyridin-2(1H)-one | 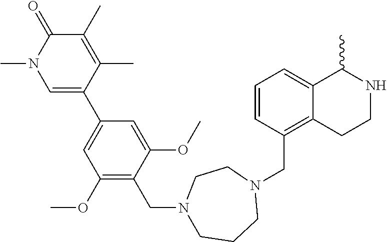 [SMILES: COc1cc(-c2cn(C)c(=O)c(C)c2C)cc(OC)c1CN1CCCN(Cc2cccc3c2CCNC3C)CC1] | 545.4/87% |
| | single enantiomer of unknown absolute configuration | |
| | (using peak 2 of tert-butyl 5-bromo-1-methyl-3,4- | |
| | dihydroisoquinoline-2(1H)-carboxylate) | |
| 5-(3,5-dimethoxy-4-((7-((1-methyl- 1,2,3,4-tetrahydroisoquinolin-5- yl)methyl)-4,7-diazaspiro[2.5]octan-4- yl)methyl)phenyl)-1,3,4- trimethylpyridin-2(1H)-one | 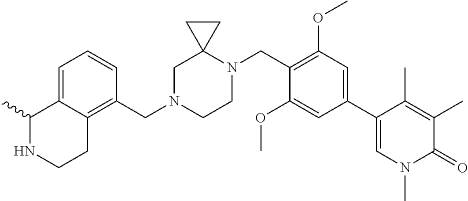 | 557.1/78% |
| | single enantiomer of unknown absolute configuration | |
| | (using peak 2 of tert-butyl 5-bromo-1-methyl-3,4- | |
| | dihydroisoquinoline-2(1H)-carboxylate) | |
| 5-(3-fluoro-5-methoxy-4-((4-((1- methyl-1,2,3,4-tetrahydroisoquinolin- 7-yl)methyl)-4,7-diazaspiro[2.5]octan- 7-yl)methyl)phenyl)-1,3,4- trimethylpyridin-2(1H)-one | 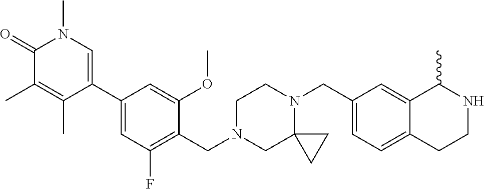 [SMILES: COc1cc(-c2cn(C)c(=O)c(C)c2C)cc(F)c1CN1CCN(Cc2ccc3c(c2)C(C)NCC3)C2(CC2)C1] | 545.4/79% |
| | single enantiomer of unknown absolute configuration | |
| | (using peak 1 of tert-butyl 7-bromo-1-methyl-3,4- | |
| | dihydroisoquinoline-2(1H)-carboxylate) | |
| 4-(3,5-dimethoxy-4-((4-((1-methyl- 1,2,3,4-tetrahydroisoquinolin-7- yl)methyl)-4,7-diazaspiro[2.5]octan-7- yl)methyl)phenyl)-6-methyl-1,6- dihydro-7H-pyrazolo[3,4-c]pyridin-7- one | 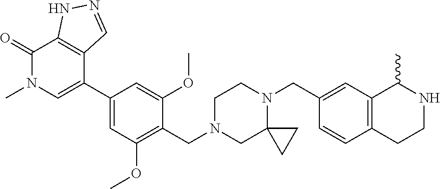 [SMILES: COc1cc(-c2cn(C)c(=O)c3[nH]ncc23)cc(OC)c1CN1CCN(Cc2ccc3c(c2)C(C)NCC3)C2(CC2)C1] | 569.3/98% |
| | single enantiomer of unknown absolute configuration | |
| | (using peak 1 of tert-butyl 7-bromo-1-methyl-3,4- | |
| | dihydroisoquinoline-2(1H)-carboxylate) | |
| 5-(4-((4-((1-isopropyl- 1,2,3,4-tetrahydroisoquinolin-5- yl)methyl)piperazin-1-yl)methyl)-3,5- dimethoxyphenyl)-1,3,4- trimethylpyridin-2(1H)-one | 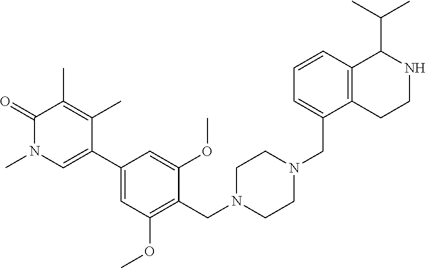 [SMILES: COc1cc(-c2cn(C)c(=O)c(C)c2C)cc(OC)c1CN1CCN(Cc2cccc3c2CCNC3C(C)C)CC1] | 558.4/89% |
| 5-(4-((4-((1-isopropyl- 1,2,3,4-tetrahydroisoquinolin-5-yl) methyl)-4,7-diazaspiro[2.5]octan-7-yl) methyl)-3,5-dimethoxyphenyl)-1,3,4- trimethylpyridin-2(1H)-one | 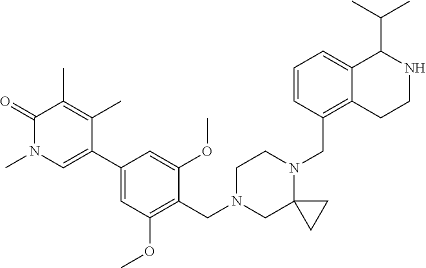 [SMILES: COc1cc(-c2cn(C)c(=O)c(C)c2C)cc(OC)c1CN1CCN(Cc2cccc3c2CCNC3C(C)C)C2(CC2)C1] | 585.4/92% |
| 5-(4-((4-((1,1-dimethyl- 1,2,3,4-tetrahydroisoquinolin-5-yl)methyl)- 4,7-diazaspiro[2.5]octan-7-yl)methyl)- 3,5-dimethoxyphenyl)-1,3,4- trimethylpyridin-2(1H)-one | 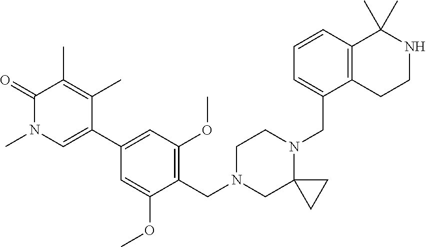 [SMILES: COc1cc(-c2cn(C)c(=O)c(C)c2C)cc(OC)c1CN1CCN(Cc2cccc3c2CCNC3(C)C)C2(CC2)C1] | 571.2/99% |
| 5-(4-((7-((1-isopropyl- 1,2,3,4-tetrahydroisoquinolin-5-yl)methyl)- 4,7-diazaspiro[2.5]octan-4-yl)methyl)- 3,5-dimethoxyphenyl)-1,3,4- trimethylpyridin-2(1H)-one | 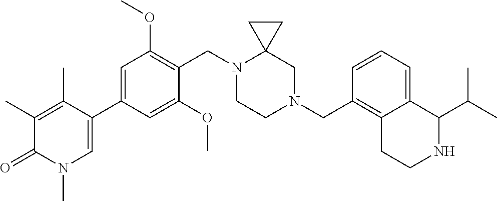 [SMILES: COc1cc(-c2cn(C)c(=O)c(C)c2C)cc(OC)c1CN1CCN(Cc2cccc3c2CCNC3C(C)C)CC12CC2] | 585.4/93% |
| 5-(4-((4-((1-isopropyl- 1,2,3,4-tetrahydroisoquinolin-5-yl) methyl)-1,4-diazepan-1-yl)methyl)-3,5- dimethoxyphenyl)-1,3,4- trimethylpyridin-2(1H)-one | 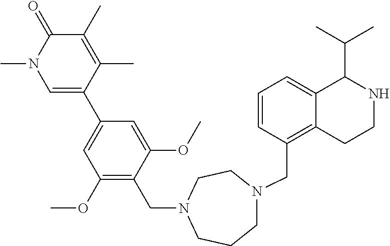 [SMILES: COc1cc(-c2cn(C)c(=O)c(C)c2C)cc(OC)c1CN1CCCN(Cc2cccc3c2CCNC3C(C)C)CC1] | 573.3/98% |
| 5-(4-(((2S,5R)-4-((1-isopropyl- 1,2,3,4-tetrahydroisoquinolin-5- yl)methyl)-2,5-dimethylpiperazin-1- yl)methyl)-3,5-dimethoxyphenyl)- 1,3,4-trimethylpyridin-2(1H)-one | 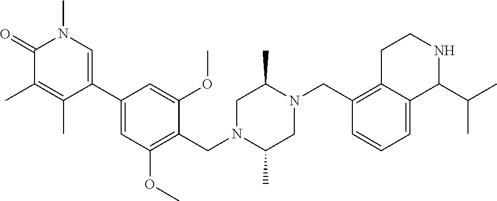 [SMILES: COc1cc(-c2cn(C)c(=O)c(C)c2C)cc(OC)c1CN1C[C@@H](C)N(Cc2cccc3c2CCNC3C(C)C)C[C@@H]1C] | 587.3/78% |
| 5-(4-((4-((1-(difluoromethyl)- 1,2,3,4-tetrahydroisoquinolin-5-yl)methyl)- 4,7-diazaspiro[2.5]octan-7-yl)methyl)- 3,5-dimethoxyphenyl)-1,3,4- trimethylpyridin-2(1H)-one | 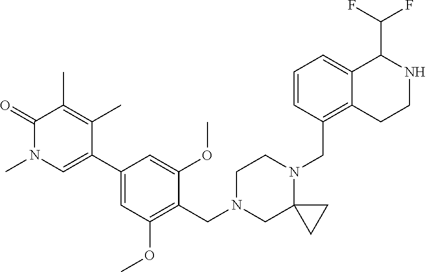 [SMILES: COc1cc(-c2cn(C)c(=O)c(C)c2C)cc(OC)c1CN1CCN(Cc2cccc3c2CCNC3C(F)F)C2(CC2)C1] | 593.4/79% |
| 5-(4-(((3S)-4-((1-isopropyl-1,2,3,4- tetrahydroisoquinolin-5-yl)methyl)-3- methylpiperazin-1-yl)methyl)-3,5- dimethoxyphenyl)-1,3,4- trimethylpyridin-2(1H)-one | 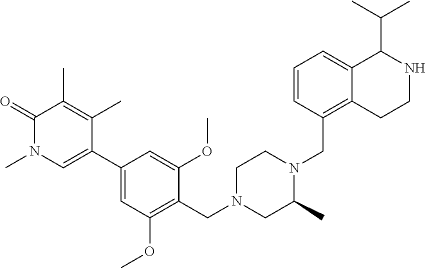 [SMILES: COc1cc(-c2cn(C)c(=O)c(C)c2C)cc(OC)c1CN1CCN(Cc2cccc3c2CCNC3C(C)C)[C@@H](C)C1] | 573.2/98% |
| 5-(4-((4-((1-ethyl- 1,2,3,4-tetrahydroisoquinolin-5-yl)methyl)- 4,7-diazaspiro[2.5]octan-7-yl)methyl)- 3,5-dimethoxyphenyl)-1,3,4- trimethylpyridin-2(1H)-one | 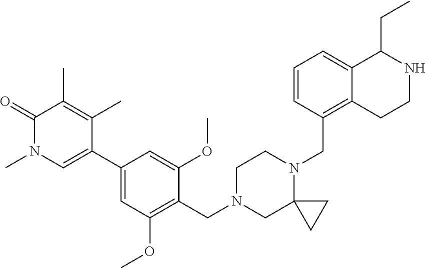 [SMILES: CCC1NCCc2c(CN3CCN(Cc4c(OC)cc(-c5cn(C)c(=O)c(C)c5C)cc4OC)CC34CC4)cccc21] | 571.2/78% |
| 5-(4-((4-((1-cyclopropyl- 1,2,3,4-tetrahydroisoquinolin-5-yl)methyl)- 4,7-diazaspiro[2.5]octan-7-yl)methyl)- 3,5-dimethoxyphenyl)-1,3,4- trimethylpyridin-2(1H)-one | 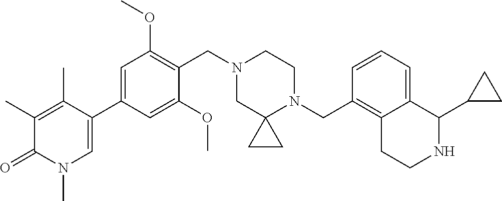 [SMILES: COc1cc(-c2cn(C)c(=O)c(C)c2C)cc(OC)c1CN1CCN(Cc2cccc3c2CCNC3C2CC2)C2(CC2)C1] | 583.4/82% |
| 5-(3,5-dimethoxy-4-((4-((1-propyl- 1,2,3,4-tetrahydroisoquinolin-5- yl)methyl)-4,7-diazaspiro[2.5]octan-7- yl)methyl)phenyl)-1,3,4-trimethylpyridin-2(1H)- one | 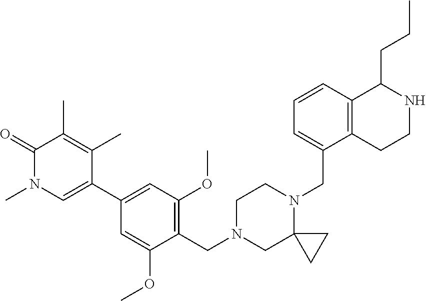 [SMILES: CCCC1NCCc2c(CN3CCN(Cc4c(OC)cc(-c5cn(C)c(=O)c(C)c5C)cc4OC)CC34CC4)cccc21] | 585.3/92% |
| 5-(3,5-dimethoxy-4-((4-((1-methoxy- 1-(methoxymethyl)- 1,2,3,4-tetrahydroisoquinolin-5-yl) methyl)-4,7-diazaspiro[2.5]octan-7- yl)methyl)phenyl)-1,3,4-trimethylpyridin- 2(1H)-one | 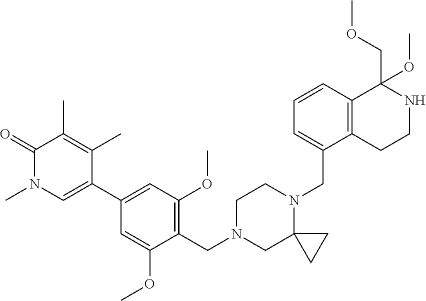 [SMILES: COCC1(OC)NCCc2c(CN3CCN(Cc4c(OC)cc(-c5cn(C)c(=O)c(C)c5C)cc4OC)CC34CC4)cccc21] | 575.2/91% |

Table 7a: The following examples were prepared by general procedure 6 using starting materials synthesized in Table 6a and (E/Z)-2-cyano-4,4-dimethylpent-2-enoic acid.

TABLE 7a

| | | LCMS |
|---|---|---|
| | | m/z |
| | | [M + H]⁺/ |
| Name | Product Structure/Name | Yield |
| BRD9a | 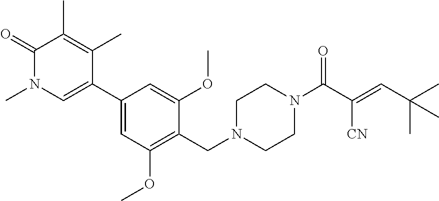 [SMILES: COc1cc(-c2cn(C)c(=O)c(C)c2C)cc(OC)c1CN1CCN(C(=O)/C(C#N)=C/C(C)(C)C)CC1] | 507.2/48% |
| | (E/Z)-2-(4-(2,6-dimethoxy-4-(1,4,5-trimethyl-6-oxo-1,6- | |
| | dihydropyridin-3-yl)benzyl)piperazine-1-carbonyl)-4,4- | |
| | dimethylpent-2-enenitrile | |
| BRD9b | 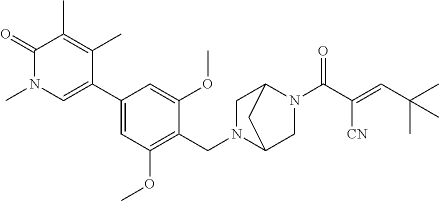 [SMILES: COc1cc(-c2cn(C)c(=O)c(C)c2C)cc(OC)c1CN1CC2CC1CN2C(=O)/C(C#N)=C/C(C)(C)C] | 519.2/27% |
| | (E/Z)-2-(5-(2,6-dimethoxy-4-(1,4,5-trimethyl-6-oxo-1,6- | |
| | dihydropyridin-3-yl)benzyl)-2,6-diazabicyclo[2.2.1]heptane- | |
| | 2-carbonyl)-4,4-dimethylpent-2- | |
| | enenitrile | |
| BRD9c | 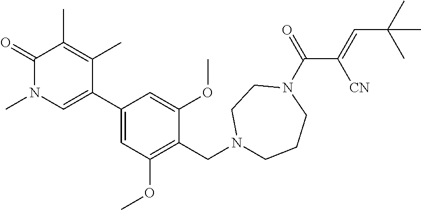 [SMILES: COc1cc(-c2cn(C)c(=O)c(C)c2C)cc(OC)c1CN1CCCN(C(=O)/C(C#N)=C/C(C)(C)C)CC1] | 521.2/48% |
| | (E/Z)-2-(4-(2,6-dimethoxy-4-(1,4,5-trimethyl-6-oxo-1,6- | |
| | dihydropyridin-3-yl)benzyl)-1,4-diazepane-1-carbonyl)- | |
| | 4,4-dimethylpent-2-enenitrile | |
| BRD9d | 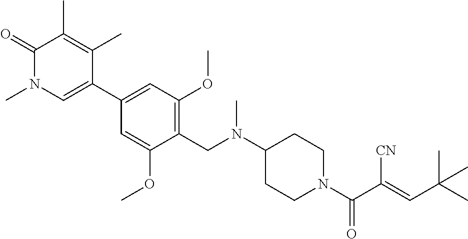 [SMILES: COc1cc(-c2cn(C)c(=O)c(C)c2C)cc(OC)c1CN(C)C1CCN(C(=O)/C(C#N)=C/C(C)(C)C)CC1] | 535.6/24% |
| | (E/Z)-2-(4-((2,6-dimethoxy-4-(1,4,5-trimethyl-6-oxo- | |
| | 1,6-dihydropyridin-3-yl)benzyl)(methyl)amino)piperidine- | |
| | 1-carbonyl)-4,4-dimethylpent-2-enenitrile | |
| BRD9e | 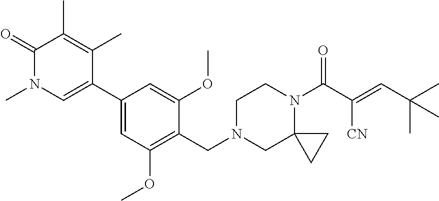 [SMILES: COc1cc(-c2cn(C)c(=O)c(C)c2C)cc(OC)c1CN1CCN(C(=O)/C(C#N)=C/C(C)(C)C)C2(CC2)C1] | 533.2/49% |
| | (E/Z)-2-(7-(2,6-dimethoxy-4-(1,4,5-trimethyl-6-oxo-1,6- | |
| | dihydropyridin-3-yl)benzyl)-4,7-diazaspiro[2,5]octane- | |
| | 4-carbonyl)-4,4-dimethylpent-2-enenitrile | |
| BRD9f | 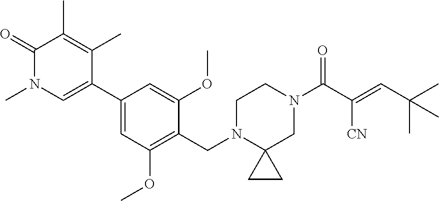 [SMILES: COc1cc(-c2cn(C)c(=O)c(C)c2C)cc(OC)c1CN1CCN(C(=O)/C(C#N)=C/C(C)(C)C)CC12CC2] | 533.3/35% |
| | (E/Z)-2-(4-(2,6-dimethoxy-4-(1,4,5-trimethyl-6-oxo-1,6- | |
| | dihydropyridin-3-yl)benzyl)-4,7-diazaspiro[2.5]octane- | |
| | 7-carbonyl)-4,4-dimethylpent-2-enenitrile | |
| BRD9ac | 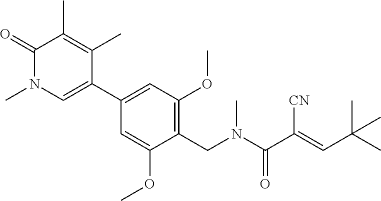 [SMILES: COc1cc(-c2cn(C)c(=O)c(C)c2C)cc(OC)c1CN(C)C(=O)/C(C#N)=C/C(C)(C)C] | 452.4/12% |
| | (E/Z)-2-cyano-N-(2,6-dimethoxy-4-(1,4,5-trimethyl- | |
| | 6-oxo-1,6-dihydropyridin-3-yl)benzyl)-N,4,4-trimethylpent- | |
| | 2-enamide | |
| B190 | 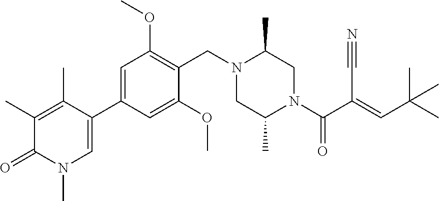 [SMILES: COc1cc(-c2cn(C)c(=O)c(C)c2C)cc(OC)c1CN1C[C@@H](C)N(C(=O)/C(C#N)=C/C(C)(C)C)C[C@@H]1C] | 535.3/18% |
| | (E/Z)-2-((2R,5S)-4-(2,6-dimethoxy-4-(1,4,5-trimethyl-6- | |
| | oxo-1,6-dihydropyridin-3-yl)benzyl)-2,6-dimethylpiperazine- | |
| | 1-carbonyl)-4,4-dimethylpent-2-enenitrile | |
| B215 | 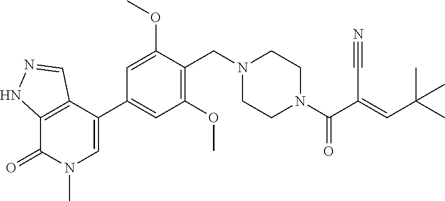 [SMILES: COc1cc(-c2cn(C)c(=O)c3[nH]ncc23)cc(OC)c1CN1CCN(C(=O)/C(C#N)=C/C(C)(C)C)CC1] | 519.2/19% |
| | (E/Z)-2-(4-(2,6-dimethoxy-4-(6-methyl-7-oxo-6,7-dihydro- | |
| | 1H-pyrazolo[3,4-c]pyridin-4-yl)benzyl)piperazine-1- | |
| | carbonyl)-4,4-dimethylpent-2-enenitrile | |
| BRD9w | 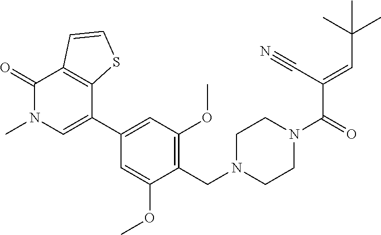 [SMILES: COc1cc(-c2cn(C)c(=O)c3ccsc23)cc(OC)c1CN1CCN(C(=O)/C(C#N)=C/C(C)(C)C)CC1] | 535.2/16% |
| | (E/Z)-2-(4-(2,6-dimethoxy-4-(5-methyl-4-oxo-4,5-dihydrothieno | |
| | [3,2-c]pyridin-7-yl)benzyl)piperazine-1-carbonyl)-4,4- | |
| | dimethylpent-2-enenitrile | |
| B65 | 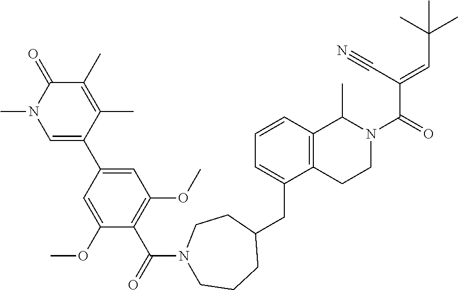 [SMILES: COc1cc(-c2cn(C)c(=O)c(C)c2C)cc(OC)c1C(=O)N1CCCC(Cc2cccc3c2CCN(C(=O)/C(C#N)=C/C(C)(C)C)C3C)CC1] | 694.3/4% |
| | (E/Z)-2-(5-((4-(2,6-dimethoxy-4-(1,4,5-trimethyl-6-oxo- | |
| | 1,6-dihydropyridin-3-yl)benzoyl)-1,4-diazepan-1-yl)methyl)- | |
| | 1-methyl-1,2,3,4-tetrahydroisoquinoline-2-carbonyl)-4,4- | |
| | dimethylpent-2-enenitrile | |
| B121 | 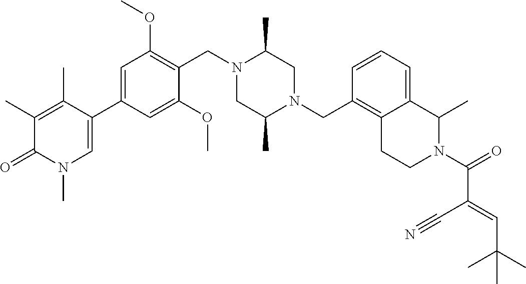 [SMILES: COc1cc(-c2cn(C)c(=O)c(C)c2C)cc(OC)c1CN1C[C@H](C)N(Cc2cccc3c2CCN(C(=O)/C(C#N)=C/C(C)(C)C)C3C)C[C@@H]1C] | 694.4/10% |
| | (E/Z)-2-(5-(((2S,5S)-4-(2,6-dimethoxy-4-(1,4,5-trimethyl-6-oxo-1,6- | |
| | dihydropyridin-3-yl)benzyl)-2,5-dimethylpiperazin-1-yl)methyl)-1-methyl- | |
| | 1,2,3,4-tetrahydroisoquinoline-2-carbonyl)-4,4-dimethylpent-2-enenitrile | |
| B122 | 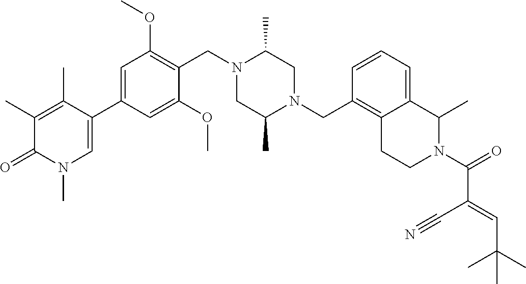 [SMILES: COc1cc(-c2cn(C)c(=O)c(C)c2C)cc(OC)c1CN1C[C@H](C)N(Cc2cccc3c2CCN(C(=O)/C(C#N)=C/C(C)(C)C)C3C)C[C@H]1C] | 694.4/11% |
| | (E/Z)-2-(5-(((2S,5R)-4-(2,6-dimethoxy-4-(1,4,5-trimethyl-6-oxo-1,6- | |
| | dihydropyridin-3-yl)benzyl)-2,5-dimethylpiperazin-1-yl)methyl)-1-methyl- | |
| | 1,2,3,4-tetrahydroisoquinoline-2-carbonyl)-4,4-dimethylpent-2-enenitrile | |
| B104 | 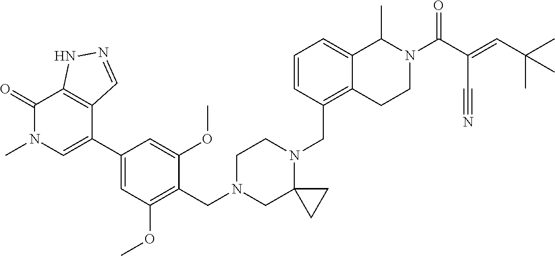 [SMILES: COc1cc(-c2cn(C)c(=O)c3[nH]ncc23)cc(OC)c1CN1CCN(Cc2cccc3c2CCN(C(=O)/C(C#N)=C/C(C)(C)C)C3C)C2(CC2)C1] | 704.4/17% |
| | (E/Z)-2-(5-((7-(2,6-dimethoxy-4-(6-methyl-7-oxo-6,7-dihydro-1H- | |
| | pyrazolo[3,4-c]pyridin-4-yl)benzyl)-4,7-diazaspiro[2.5]octan-4-yl)methyl)-1- | |
| | methyl-1,2,3,4-tetrahydroisoquinoline-2-carbonyl)-4,4-dimethylpent-2- | |
| | enenitrile | |
| B107 | 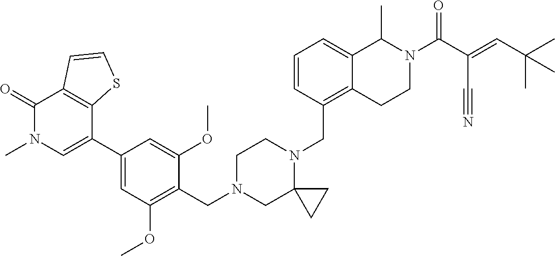 [SMILES: COc1cc(-c2cn(C)c(=O)c3ccsc23)cc(OC)c1CN1CCN(Cc2cccc3c2CCN(C(=O)/C(C#N)=C/C(C)(C)C)C3C)C2(CC2)C1] | 720.4/17% |
| | (E/Z)-2-(5-((7-(2,6-dimethoxy-4-(5-methyl-4-oxo-4,5-dihydrothieno[3,2- | |
| | c]pyridin-7-yl)benzyl)-4,7-diazaspiro[2.5]octan-4-yl)methyl)-1-methyl- | |
| | 1,2,3,4-tetrahydroisoquinoline-2-carbonyl)-4,4-dimethylpent-2-enenitrile | |
| B101 | 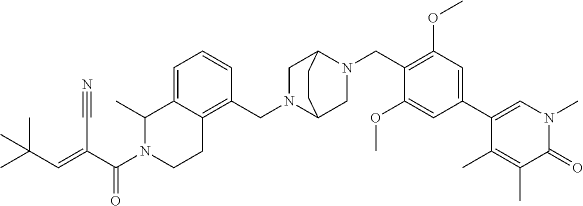 [SMILES: COc1cc(-c2cn(C)c(=O)c(C)c2C)cc(OC)c1CN1CC2CCC1CN2Cc1cccc2c1CCN(C(=O)/C(C#N)=C/C(C)(C)C)C2C] | 692.4/41% |
| | (E/Z)-2-(5-((5-(2,6-dimethoxy-4-(1,4,5-trimethyl-6-oxo-1,6-dihydropyridin- | |
| | 3-yl)benzyl)-2,5-diazabicyclo[2.2.2]octan-2-yl)methyl)-1-methyl-1,2,3,4- | |
| | tetrahydroisoquinoline-2-carbonyl)-4,4-dimethylpent-2-enenitrile | |
| B119 | 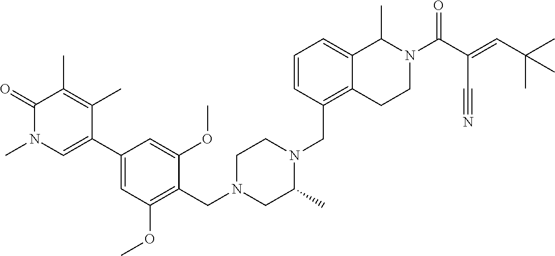 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)[C@H](C)C1] | 680.4/6% |
| | (E/Z)-2-(5-(((R)-4-(2,6-dimethoxy-4-(1,4,5-trimethyl-6-oxo-1,6- | |
| | dihydropyridin-3-yl)benzyl)-2-methylpiperazin-1-yl)methyl)-1-methyl- | |
| | 1,2,3,4-tetrahydroisoquinoline-2-carbonyl)-4,4-dimethylpent-2-enenitrile | |
| B102 | 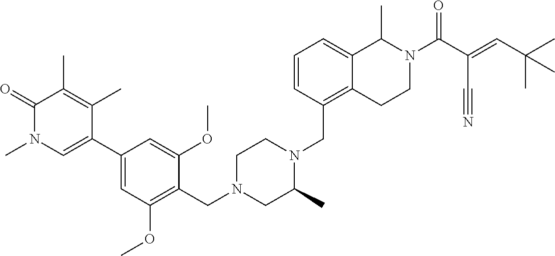 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)[C@@H](C)C1] | 680.4/31% |
| | (E/Z)-2-(5-(((S)-4-(2,6-dimethoxy-4-(1,4,5-trimethoxy-6-oxo-1,6- | |
| | dihydropyridin-3-yl)benzyl)-2-methylpiperazin-1-yl)methyl)-1-methyl- | |
| | 1,2,3,4-tetrahydroisoquinoline-2-carbonyl)-4,4-dimethylpent-2-enenitrile | |
| B114 | 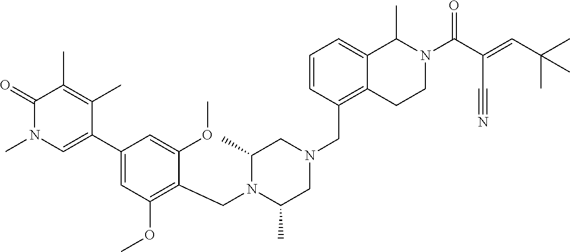 [SMILES: COc1cc(-c2cn(C)c(=O)c(C)c2C)cc(OC)c1CN1[C@H](C)CN(Cc2cccc3c2CCN(C(=O)/C(C#N)=C/C(C)(C)C)C3C)C[C@@H]1C] | 694.4/4% |
| | (E/Z)-2-(5-(((3S,5R)-4-(2,6-dimethoxy-4-(1,4,5-trimethyl-6-oxo-1,6- | |
| | dihydropyridin-3-yl)benzyl)-3,5-dimethylpiperazin-1-yl)methyl)-1-methyl- | |
| | 1,2,3,4-tetrahydroisoquinoline-2-carbonyl)-4,4-dimethylpent-2-enenitrile | |
| B103 | 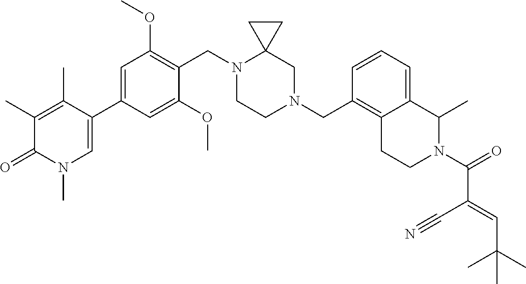 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)CC12CC2] | 692.4/40% |
| | (E/Z)-2-(5-((4-(2,6-dimethoxy-4-(1,4,5-trimethyl-6-oxo-1,6-dihydropyridin- | |
| | 3-yl)benzyl)-4,7-diazaspiro[2.5]octan-7-yl)methyl)-1-methyl-1,2,3,4- | |
| | tetrahydroisoquinoline-2-carbonyl)-4,4-dimethylpent-2-enenitrile | |
| B115 | 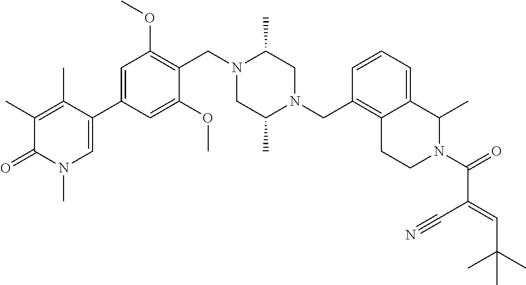 [SMILES: COc1cc(-c2cn(C)c(=O)c(C)c2C)cc(OC)c1CN1C[C@@H](C)N(Cc2cccc3c2CCN(C(=O)/C(C#N)=C/C(C)(C)C)C3C)C[C@H]1C] | 694.4/23% |
| | (E/Z)-2-(5-(((2R,5R)-4-(2,6-dimethoxy-4-(1,4,5-trimethyl-6-oxo-1,6- | |
| | dihydropyridin-3-yl)benzyl)-2,5-dimethylpiperazin-1-yl)methyl)-1-methyl- | |
| | 1,2,3,4-tetrahydroisoquinoline-2-carbonyl)-4,4-dimethylpent-2-enenitrile | |
| B136 | 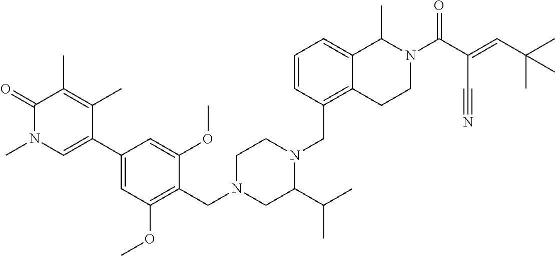 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)C(C(C)C)C1] | 708.2/27% |
| | (E/Z)-2-(5-((4-(2,6-dimethoxy-4-(1,4,5-trimethyl-6-oxo-1,6-dihydropyridin- | |
| | 3-yl)benzyl)-2-isopropylpiperazin-1-yl)methyl)-1-methyl-1,2,3,4- | |
| | tetrahydroisoquinoline-2-carbonyl)-4,4-dimethylpent-2-enenitrile | |
| B139 | 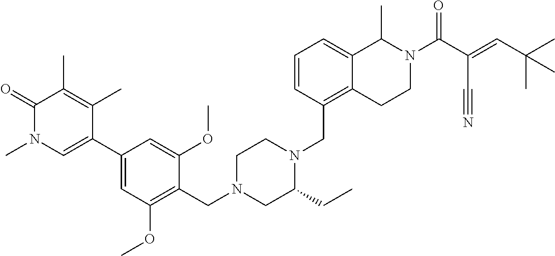 [SMILES: CC[C@@H]1CN(Cc2c(OC)cc(-c3cn(C)c(=O)c(C)c3C)cc2OC)CCN1Cc1cccc2c1CCN(C(=O)/C(C#N)=C/C(C)(C)C)C2C] | 694.4/28% |
| | (E/Z)-2-(5-(((R)-4-(2,6-dimethoxy-4-(1,4,5-trimethyl-6-oxo-1,6- | |
| | dihydropyridin-3-yl)benzyl)-2-ethylpiperazin-1-yl)methyl)-1-methyl-1,2,3,4- | |
| | tetrahydroisoquinoline-2-carbonyl)-4,4-dimethylpent-2-enenitrile | |
| B113 | 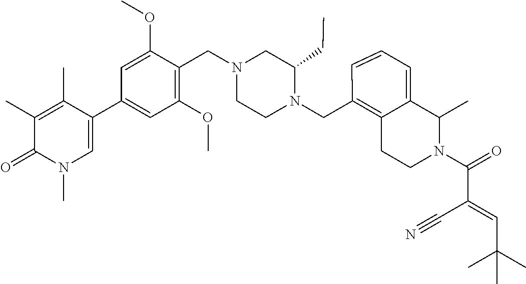 [SMILES: CC[C@H]1CN(Cc2c(OC)cc(-c3cn(C)c(=O)c(C)c3C)cc2OC)CCN1Cc1cccc2c1CCN(C(=O)/C(C#N)=C/C(C)(C)C)C2C] | 694.4/19% |
| | (E/Z)-2-(5-(((S)-4-(2,6-dimethoxy-4-(1,4,5-trimethyl-6-oxo-1,6- | |
| | dihydropyridin-3-yl)benzyl)-2-ethylpiperazin-1-yl)methyl)-1-methyl-1,2,3,4- | |
| | tetrahydroisoquinoline-2-carbonyl)-4,4-dimethylpent-2-enenitrile | |
| B100 | 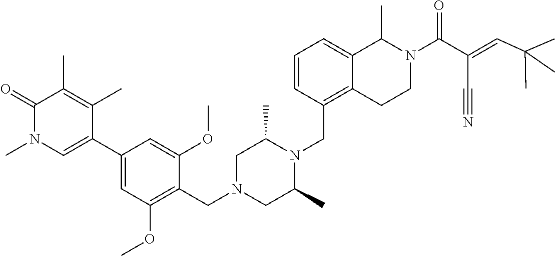 [SMILES: COc1cc(-c2cn(C)c(=O)c(C)c2C)cc(OC)c1CN1C[C@H](C)N(Cc2cccc3c2CCN(C(=O)/C(C#N)=C/C(C)(C)C)C3C)[C@@H](C)C1] | 694.4/35% |
| | (E/Z)-2-(5-(((2S,6S)-4-(2,6-dimethoxy-4-(1,4,5-trimethyl-6-oxo-1,6- | |
| | dihydropyridin-3-yl)benzyl)-2,6-dimethylpiperazin-1-yl)methyl)-1-methyl- | |
| | 1,2,3,4-tetrahydroisoquinoline-2-carbonyl)-4,4-dimethylpent-2-enenitrile | |
| B135 | 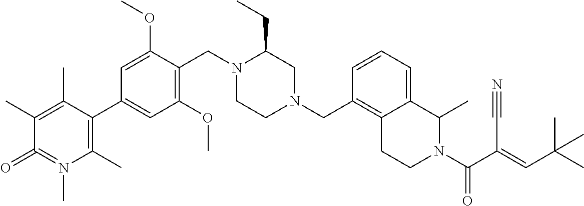 | 694.4/11% |
| | (E/Z)-2-(5-(((S)-4-(2,6-dimethoxy-4-(1,4,5-trimethyl-6-oxo-1,6- | |
| | dihydropyridin-3-yl)benzyl)-3-ethylpiperazin-1-yl)methyl)-1-methyl-1,2,3,4- | |
| | tetrahydroisoquinoline-2-carbonyl)-4,4-dimethylpent-2-enenitrile | |
| B148 | 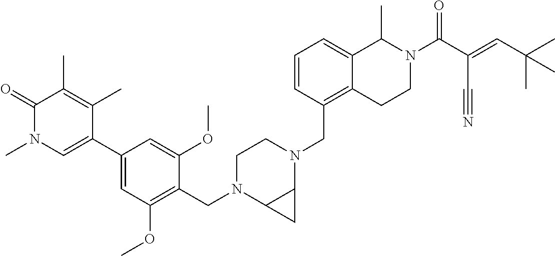 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)C2CC21] | 678.4/7% |
| | (E/Z)-2-(5-((5-(2,6-dimethoxy-4-(1,4,5-trimethyl-6-oxo-1,6-dihydropyridin- | |
| | 3-yl)benzyl)-2,5-diazabicyclo[4.1.0]heptan-2-yl)methyl)-1-methyl-1,2,3,4- | |
| | tetrahydroisoquinoline-2-carbonyl)-4,4-dimethylpent-2-enenitrile | |
| B111 | 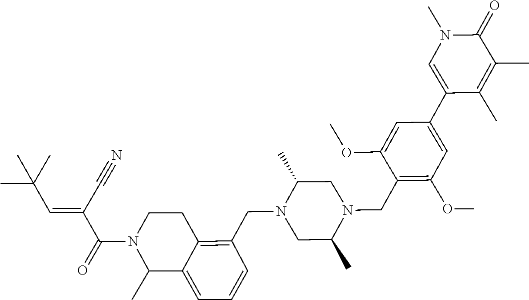 [SMILES: COc1cc(-c2cn(C)c(=O)c(C)c2C)cc(OC)c1CN1C[C@@H](C)N(Cc2cccc3c2CCN(C(=O)/C(C#N)=C/C(C)(C)C)C3C)C[C@@H]1C] | 694.4/38% |
| | (E/Z)-2-(5-(((2R,5S)-4-(2,6-dimethoxy-4-(1,4,5-trimethyl-6-oxo-1,6- | |
| | dihydropyridin-3-yl)benzyl)-2,5-dimethylpiperazin-1-yl)methyl)-1-methyl- | |
| | 1,2,3,4-tetrahydroisoquinoline-2-carbonyl)-4,4-dimethylpent-2-enenitrile | |
| B130 | 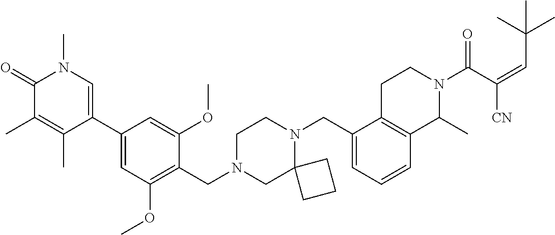 [SMILES: COc1cc(-c2cn(C)c(=O)c(C)c2C)cc(OC)c1CN1CCN(Cc2cccc3c2CCN(C(=O)/C(C#N)=C\C(C)(C)C)C3C)C2(CCC2)C1] | 706.4/40% |
| | (E/Z)-2-(5-((8-(2,6-dimethoxy-4-(1,4,5-trimethyl-6-oxo-1,6-dihydropyridin- | |
| | 3-yl)benzyl)-5,8-diazaspiro[3.5]nonan-5-yl)methyl)-1-methyl-1,2,3,4- | |
| | tetrahydroisoquinoline-2-carbonyl)-4,4-dimethylpent-2-enenitrile | |
| B99 | 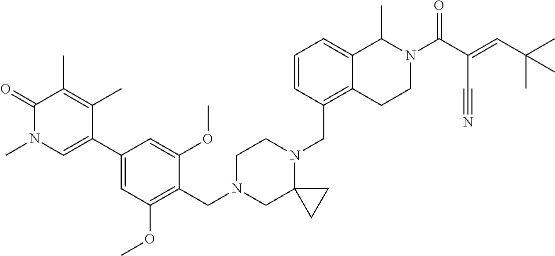 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)C2(CC2)C1] | 706.4/6% |
| | (E/Z)-3-(6-bromopyridin-2-yl)-2-(5-((4-(2,6-dimethoxy-4-(1,4,5-trimethyl-6- | |
| | oxo-1,6-dihydropyridin-3-yl)benzyl)-2,2-dimethylpiperazin-1-yl)methyl)-1- | |
| | methyl-1,2,3,4-tetrahydroisoquinoline-2-carbonyl)acrylonitrile | |
| B98 | 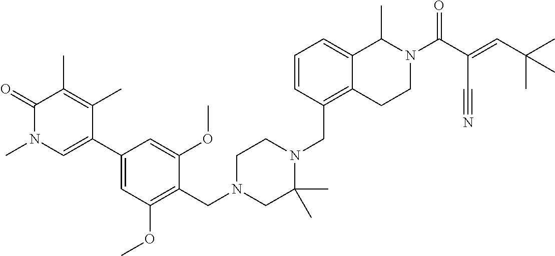 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)C(C)(C)C1] | 694.4/28% |
| | (E/Z)-2-(5-((4-(2,6-dimethoxy-4-(1,4,5-trimethyl-6-oxo-1,6-dihydropyridin- | |
| | 3-yl)benzyl)-2,2-dimethylpiperazin-1-yl)methyl)-1-methyl-1,2,3,4- | |
| | tetrahydroisoquinoline-2-carbonyl)-4,4-dimethylpent-2-enenitrile | |
| B59 | 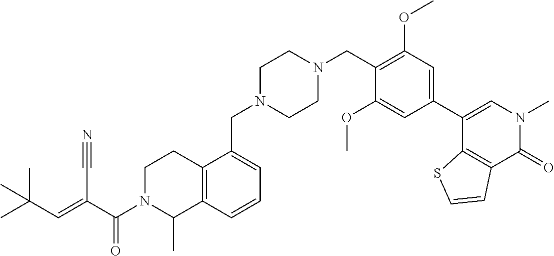 [SMILES: COc1cc(-c2cn(C)c(=O)c3ccsc23)cc(OC)c1CN1CCN(Cc2cccc3c2CCN(C(=O)/C(C#N)=C/C(C)(C)C)C3C)CC1] | 694.3/32% |
| | (E/Z)-2-(5-((4-(2,6-dimethoxy-4-(5-methyl-4-oxo-4,5-dihydrothieno[3,2- | |
| | c]pyridin-7-yl)benzyl)piperazin-1-yl)methyl)-1-methyl-1,2,3,4- | |
| | tetrahydroisoquinoline-2-carbonyl)-4,4-dimethypent-2-enenitrile | |
| B60 | 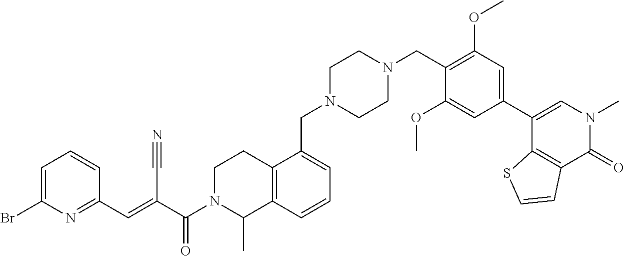 [SMILES: COc1cc(-c2cn(C)c(=O)c3ccsc23)cc(OC)c1CN1CCN(Cc2cccc3c2CCN(C(=O)/C(C#N)=C/c2cccc(Br)n2)C3C)CC1] | 708.4/42% |
| | (E)-3-(6-bromopyridin-2-yl)-2-(5-((4-(2,6-dimethoxy-4-(5-methyl-4-oxo-4,5- | |
| | dihydrothieno[3,2-c]pyridin-7-yl)benzyl)piperazin-1-yl)methyl)-1-methyl- | |
| | 1,2,3,4-tetrahydroisoquinoline-2-carbonyl)acrylonitrile | |
| B120 | 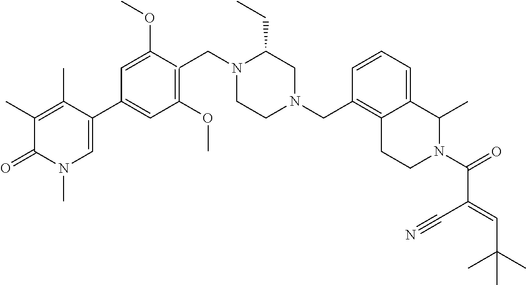 [SMILES: CC[C@@H]1CN(Cc2cccc3c2CCN(C(=O)/C(C#N)=C/C(C)(C)C)C3C)CCN1C/C=C(/C=C(\C=C(/C)OC)c1cn(C)c(=O)c(C)c1C)OC] | 694.4/13% |
| | (E/Z)-2-(5-(((R)-4-(2,6-dimethoxy-4-(1,4,5-trimethyl-6-oxo-1,6- | |
| | dihydropyridin-3-yl)benzyl)-3-ethylpiperazin-1-yl)methyl)-1-methyl-1,2,3,4- | |
| | tetrahydroisoquinoline-2-carbonyl)-4,4-dimethylpent-2-enenitrile | |
| B47 | 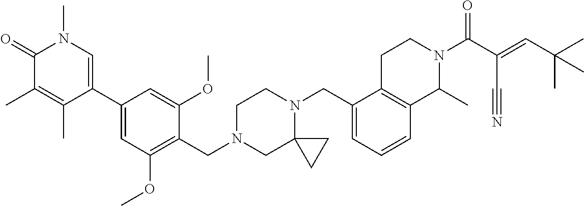 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)C2(CC2)C1] | 692.3/19% |
| | (E/Z)-2-(5-((7-(2,6-dimethoxy-4-(1,4,5-trimethyl-6-oxo-1,6-dihydropyridin- | |
| | 3-yl)benzyl)-4,7-diazaspiro[2.5]octan-4-yl)methyl)-1-methyl-1,2,3,4- | |
| | tetrahydroisoquinoline-2-carbonyl)-4,4-dimethylpent-2-enenitrile | |
| B159 | 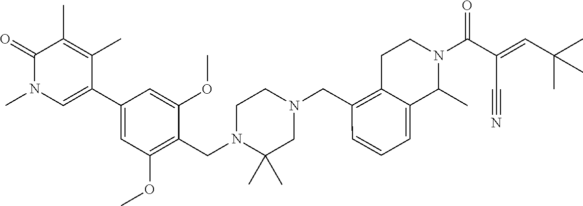 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)CC1(C)C] | 694.4/22% |
| | (E/Z)-2-(5-((4-(2,6-dimethoxy-4-(1,4,5-trimethyl-6-oxo-1,6-dihydropyridin- | |
| | 3-yl)benzyl)-3,3-dimethylpiperazin-1-yl)methyl)-1-methyl-1,2,3,4- | |
| | tetrahydroisoquinoline-2-carbonyl)-4,4-dimethylpent-2-enenitrile | |
| C106 | 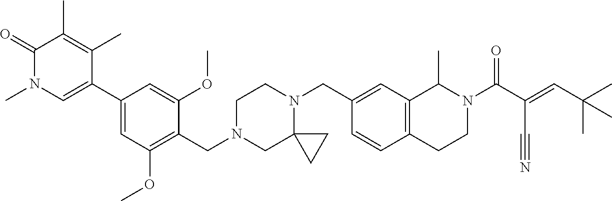 [SMILES: COc1cc(-c2cn(C)c(=O)c(C)c2C)cc(OC)c1CN1CCN(Cc2ccc3c(c2)C(C)N(C(=O)/C(C#N)=C/C(C)(C)C)CC3)C2(CC2)C1] | 692.4/13% |
| | (E/Z)-2-(7-((7-(2,6-dimethoxy-4-(1,4,5-trimethyl-6-oxo-1,6-dihydropyridin- | |
| | 3-yl)benzyl)-4,7-diazaspiro[2.5]octan-4-yl)methyl)-1-methyl-1,2,3,4- | |
| | tetrahydroisoquinoline-2-carbonyl)-4,4-dimethylpent-2-enenitrile | |
| C1 | 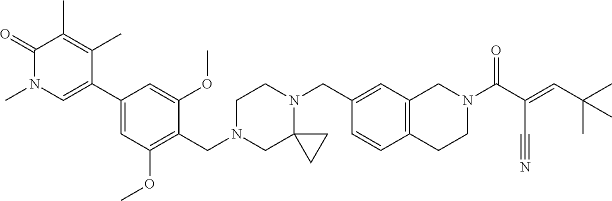 [SMILES: COc1cc(-c2cn(C)c(=O)c(C)c2C)cc(OC)c1CN1CCN(Cc2ccc3c(c2)CN(C(=O)/C(C#N)=C/C(C)(C)C)CC3)C2(CC2)C1] | 678.4/13% |
| | (E/Z)-2-(7-((7-(2,6-dimethoxy-4-(1,4,5-trimethyl-6-oxo-1,6-dihydropyridin- | |
| | 3-yl)benzyl)-4,7-diazaspiro[2.5]octan-4-yl)methyl)-1,2,3,4- | |
| | tetrahydroisoquinoline-2-carbonyl)-4,4-dimethylpent-2-enenitrile | |
| C2 | 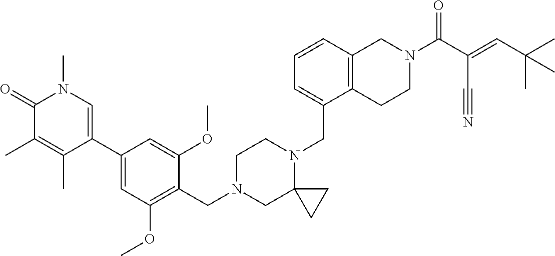 [SMILES: COc1cc(-c2cn(C)c(=O)c(C)c2C)cc(OC)c1CN1CCN(Cc2cccc3c2CCN(C(=O)/C(C#N)=C/C(C)(C)C)C3)C2(CC2)C1] | 678.3/49% |
| | (E/Z)-2-(5-((7-(2,6-dimethoxy-4-(1,4,5-trimethyl-6-oxo-1,6-dihydropyridin- | |
| | 3-yl)benzyl)-4,7-diazaspiro[2.5]octan-4-yl)methyl)-1,2,3,4- | |
| | tetrahydroisoquinoline-2-carbonyl)-4,4-dimethylpent-2-enenitrile | |
| C3 | single enantiomer of absolute | 680.2/41% |
| | unknown configuration | |
| | 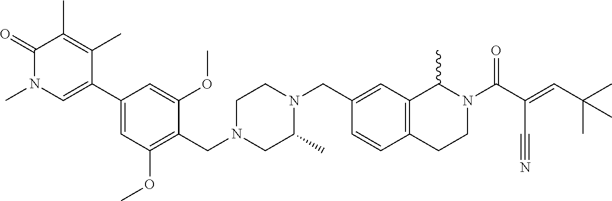 [SMILES: COc1cc(-c2cn(C)c(=O)c(C)c2C)cc(OC)c1CN1CCN(Cc2ccc3c(c2)C(C)N(C(=O)/C(C#N)=C/C(C)(C)C)CC3)[C@H](C)C1] | |
| | Single diastereomer of (E/Z)-2-(7-(((R)-4-(2,6-dimethoxy-4-(1,4,5-trimethyl- | |
| | 6-oxo-1,6-dihydropyridin-3-yl)benzyl)-2-methylpiperazin-1-yl)methyl)-1- | |
| | methyl-1,2,3,4-tetrahydroisoquinoline-2-carbonyl)-4,4-dimethylpent-2- | |
| | enentrile | |
| BRD9s | 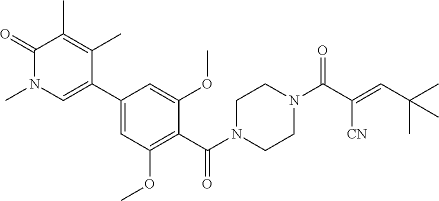 [SMILES: COc1cc(-c2cn(C)c(=O)c(C)c2C)cc(OC)c1C(=O)N1CCN(C(=O)/C(C#N)=C/C(C)(C)C)CC1] | 521.2/25% |
| | (E/Z)-2-(4-(2,6-dimethoxy-4-(1,4,5-trimethyl-6-oxo-1,6-dihydropyridin-3- | |
| | yl)benzoyl)piperazine-1-carbonyl)-4,4-dimethylpent-2-enenitrile | |
| BRD9t | 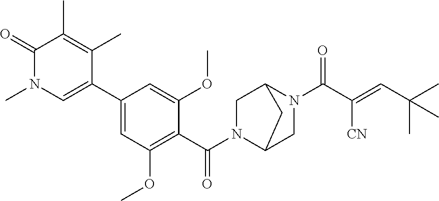 [SMILES: COc1cc(-c2cn(C)c(=O)c(C)c2C)cc(OC)c1C(=O)N1CC2CC1CN2C(=O)/C(C#N)=C/C(C)(C)C] | 533.4/12% |
| | (E/Z)-2-(5-(2,6-dimethoxy-4-(1,4,5-trimethyl-6-oxo-1,6-dihydropyridin-3- | |
| | yl)benzoyl)-2,6-diazabicyclo[2.2.1]heptane-2-carbonyl)-4,4-dimethylpent- | |
| | 2-enenitrile | |
| BRD9u | 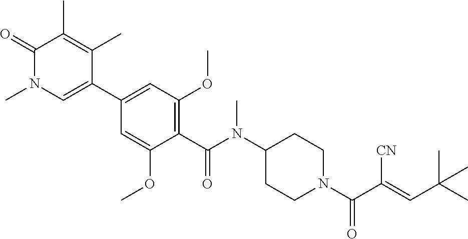 [SMILES: COc1cc(-c2cn(C)c(=O)c(C)c2C)cc(OC)c1C(=O)N(C)C1CCN(C(=O)/C(C#N)=C/C(C)(C)C)CC1] | 549.2/9% |
| | (E/Z)-2-(4-((2,6-dimethoxy-4-(1,4,5-trimethyl-6-oxo-1,6-dihydropyridin-3- | |
| | yl)benzyl)(methyl)amino)piperidine-1-carbonyl)-4,4-dimethylpent-2- | |
| | enenitrile | |
| BRD9v | 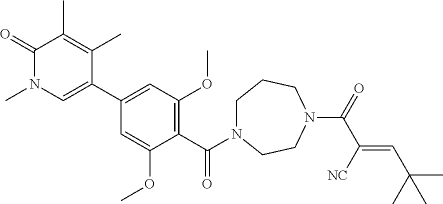 [SMILES: COc1cc(-c2cn(C)c(=O)c(C)c2C)cc(OC)c1C(=O)N1CCCN(C(=O)/C(C#N)=C/C(C)(C)C)CC1] | 535.4/18% |
| | (E/Z)-2-(4-(2,6-dimethoxy-4-(1,4,5-trimethyl-6-oxo-1,6-dihydropyridin-3- | |
| | yl)benzoyl)-1,4-diazepane-1-carbonyl)-4,4-dimethylpent-2-enenitrile | |
| B146 | 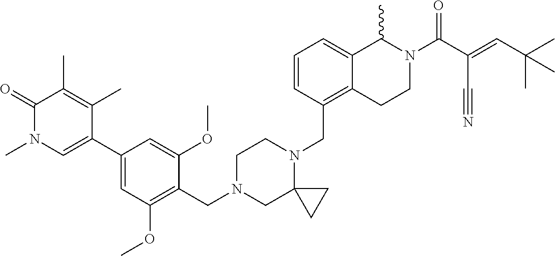 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)C2(CC2)C1] | 692.4/38% |
| | single enantiomer of unknown absolute configuration | |
| | (using peak 2 of tert-butyl 5-bromo-1-methyl-3,4- | |
| | dihydroisoquinoline-2(1H)-carboxylate) | |
| | (E/Z)-2-(5-((7-(2,6-dimethoxy-4-(1,4,5-trimethyl-6-oxo-1,6-dihydropyridin- | |
| | 3-yl)benzyl)-4,7-diazaspiro[2.5]octan-4-yl)methyl)-1-methyl-1,2,3,4- | |
| | tetrahydroisoquinoline-2-carbonyl)-4,4-dimethylpent-2-enenitrile | |
| B192 | 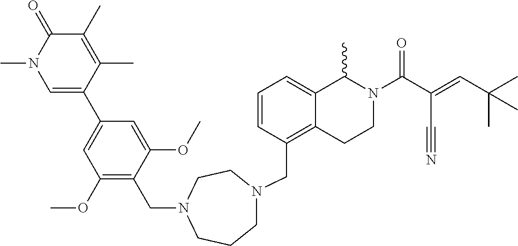 [SMILES: COc1cc(-c2cn(C)c(=O)c(C)c2C)cc(OC)c1CN1CCCN(Cc2cccc3c2CCN(C(=O)/C(C#N)=C/C(C)(C)C)C3C)CC1] | 680.4/18% |
| | single enantiomer of unknown absolute configuration | |
| | (using peak 2 of tert-butyl 5-bromo-1-methyl-3,4- | |
| | dihydroisoquinoline-2(1H)-carboxylate) | |
| | (E/Z)-2-(5-((4-(2,6-dimethoxy-4-(1,4,5-trimethyl-6-oxo-1,6-dihydropyridin- | |
| | 3-yl)benzyl)-1,4-diazepan-1-yl)methyl)-1-methyl-1,2,3,4- | |
| | tetrahydroisoquinoline-2-carbonyl)-4,4-dimethylpent-2-enenitrile | |
| C4 | 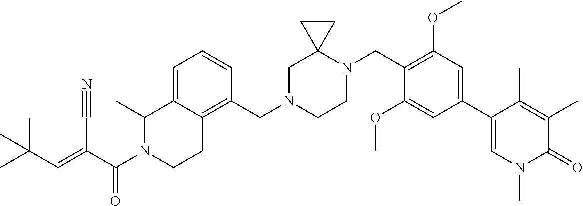 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C)CC12CC2] | 692.3/11% |
| | single enantiomer of unknown absolute configuration | |
| | (using peak 2 of tert-butyl 5-bromo-1-methyl-3,4- | |
| | dihydroisoquinoline-2(1H)-carboxylate) | |
| | (E/Z)-2-(5-((4-(2,6-dimethoxy-4-(1,4,5-trimethyl-6-oxo-1,6-dihydropyridin- | |
| | 3-yl)benzyl)-4,7-diazaspiro[2.5]octan-7-yl)methyl)-1-methyl-1,2,3,4- | |
| | tetrahydroisoquinoline-2-carbonyl)-4,4-dimethylpent-2-enenitrile | |
| C5 | 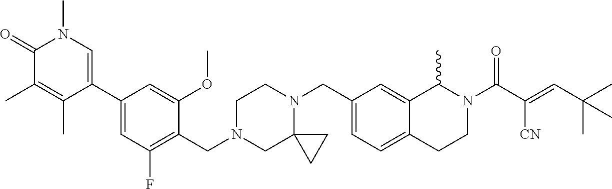 [SMILES: COc1cc(-c2cn(C)c(=O)c(C)c2C)cc(F)c1CN1CCN(Cc2ccc3c(c2)C(C)N(C(=O)/C(C#N)=C/C(C)(C)C)CC3)C2(CC2)C1] | 680.3/13% |
| | single enantiomer of unknown absolute configuration | |
| | (using peak 1 of tert-butyl 7-bromo-1-methyl-3,4- | |
| | dihydroisoquinoline-2(1H)-carboxylate) | |
| | (E/Z)-2-(7-((7-(2-fluoro-6-mehtoxy-4-(1,4,5-trimethyl-6-oxo-1,6- | |
| | dihydropyridin-3-yl)benzyl)-4,7-diazaspiro[2.5]octan-4-yl)methyl)-1-methyl- | |
| | 1,2,3,4-tetrahydroisoquinoline-2-carbonyl)-4,4-dimethylpent-2-enenitrile | |
| C6 | 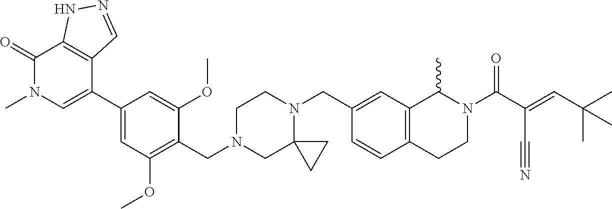 [SMILES: COc1cc(-c2cn(C)c(=O)c3[nH]ncc23)cc(OC)c1CN1CCN(Cc2ccc3c(c2)C(C)N(C(=O)/C(C#N)=C/C(C)(C)C)CC3)C2(CC2)C1] | 704.2/14% |
| | (E/Z)-2-(7-((7-(2,6-dimethoxy-4-(6-methyl-7-oxo-6,7-dihydro-1H- | |
| | pyrazolo[3,4-c]pyridin-4-yl)benzyl)-4,7-diazaspiro[2.5]octan-4-yl)methyl)-1- | |
| | methyl-1,2,3,4-tetrahydroisoquinoline-2-carbonyl)-4,4-dimethylpent-2- | |
| | enenitrile | |
| C7 | 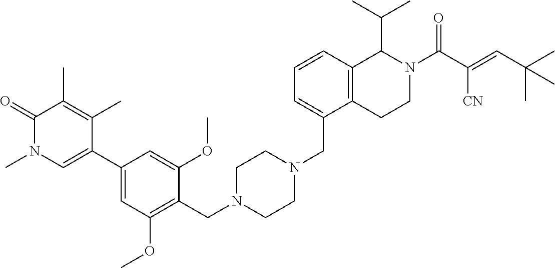 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C(C)C)CC1] | 694.4/15% |
| | (E/Z)-2-(5-((4-(2,6-dimethoxy-4-(1,4,5-trimethyl-6-oxo-1,6-dihydropyridin- | |
| | 3-yl)benzyl)piperazin-1-yl)methyl)-1-isopropyl-1,2,3,4- | |
| | tetrahydroisoquinoline-2-carbonyl)-4,4-dimethylpent-2-enenitrile | |
| C8 | 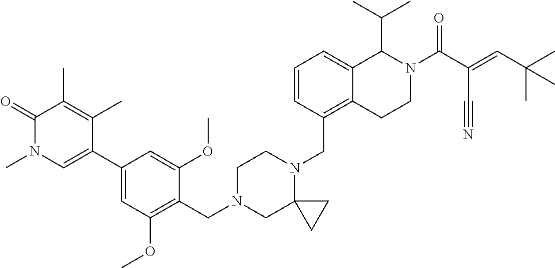 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C(C)C)C2(CC2)C1] | 720.4/32% |
| | (E/Z)-2-(5-((7-(2,6-dimethoxy-4-(1,4,5-trimethyl-6-oxo-1,6-dihydropyridin- | |
| | 3-yl)benzyl)-4,7-diazaspiro[2.5]octan-4-yl)methyl)-1-isopropyl-1,2,3,4- | |
| | tetrahydroisoquinoline-2-carbonyl)-4,4-dimethylpent-2-enenitrile | |
| C9 | 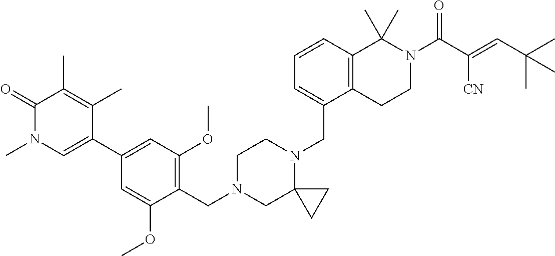 [SMILES: COc1cc(-c2cn(C)c(=O)c(C)c2C)cc(OC)c1CN1CCN(Cc2cccc3c2CCN(C(=O)/C(C#N)=C/C(C)(C)C)C3(C)C)C2(CC2)C1] | 706.4/77% |
| | (E/Z)-2-(5-((7-(2,6-dimethoxy-4-(1,4,5-trimethyl-6-oxo-1,6-dihydropyridin- | |
| | 3-yl)benzyl)-4,7-diazaspiro[2.5]octan-4-yl)methyl)-1,1-dimethyl-1,2,3,4- | |
| | tetrahydroisoquinoline-2-carbonyl)-4,4-dimethylpent-2-enenitrile | |
| C10 | 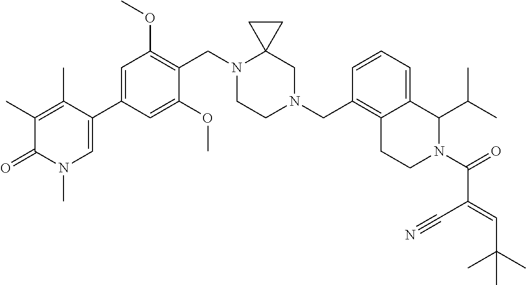 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C(C)C)CC12CC2] | 720.4/29% |
| | (E/Z)-2-(5-((4-(2,6-dimethoxy-4-(1,4,5-trimethyl-6-oxo-1,6-dihydropyridin- | |
| | 3-yl)benzyl)-4,7-diazspiro[2.5-]octan-7-yl)methyl)-1-isopropyl-1,2,3,4- | |
| | tetrahydroisoquinoline-2-carbonyl)-4,4-dimethylpent-2-enenitrile | |
| C11 | 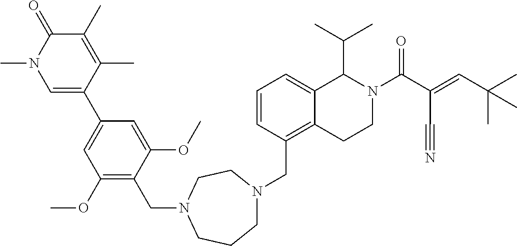 [SMILES: COc1cc(-c2cn(C)c(=O)c(C)c2C)cc(OC)c1CN1CCCN(Cc2cccc3c2CCN(C(=O)/C(C#N)=C/C(C)(C)C)C3C(C)C)CC1] | 708.4/14% |
| | (E/Z)-2-(5-((4-(2,6-dimethoxy-4-(1,4,5-trimethyl-6-oxo-1,6-dihydropyridin- | |
| | 3-yl)benzyl)-1,4-diazepan-1-yl)methyl)-1-isopropyl-1,2,3,4- | |
| | tetrahydroisoquinoline-2-carbonyl)-4,4-dimethylpent-2-enenitrile | |
| C12 | 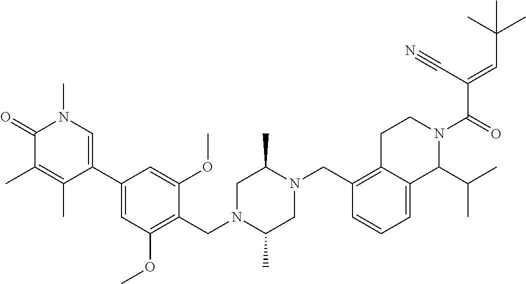 [SMILES: COc1cc(-c2cn(C)c(=O)c(C)c2C)cc(OC)c1CN1C[C@@H](C)N(Cc2cccc3c2CCN(C(=O)/C(C#N)=C/C(C)(C)C)C3C(C)C)C[C@@H]1C] | 722.4/28% |
| | (E/Z)-2-(5-(((2S,5R)-4-(2,6-dimethoxy-4-(1,4,5-trimethyl-6-oxo-1,6- | |
| | dihydropyridin-3-yl)benzyl)-2,5-dimethylpiperazin-1-yl)methyl)-1-isopropyl- | |
| | 1,2,3,4-tetrahydroisoquinoline-2-carbonyl)-4,4-dimethylpent-2-enenitrile | |
| C13 | 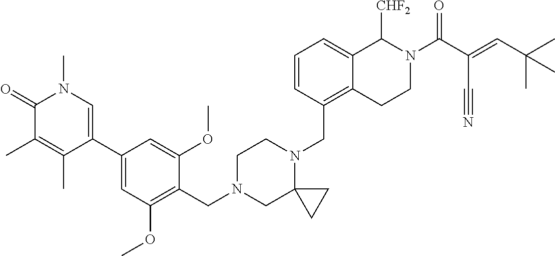 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C(F)F)C2(CC2)C1] | 728.4/7% |
| | (E/Z)-2-(1-(difluoromethyl)-5-((4-(2,6-dimethoxy-4-(1,4,5-trimethyl-6-oxo- | |
| | 1,6-dihydropyridin-3-yl)benzyl)-4,7-diazaspiro[2.5]octan-7-yl)methyl)- | |
| | 1,2,3,4-tetrahydroisoquinoline-2-carbonyl)-4,4-dimethylpent-2-enenitrile | |
| C14 | 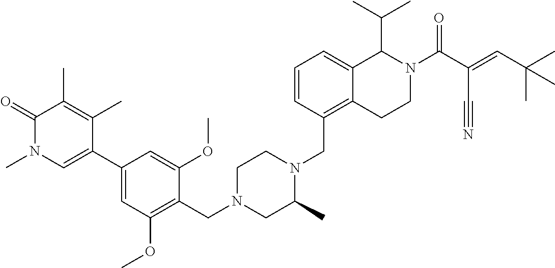 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C(C)C)[C@@H](C)C1] | 708.4/50% |
| | (E/Z)-2-(5-(((S)-4-(2,6-dimethoxy-4-(1,4,5-trimethyl-6-oxo-1,6- | |
| | dihydropyridin-3-yl)benzyl)-2-methylpiperazin-1-yl)methyl)-1-isopropyl- | |
| | 1,2,3,4-tetrahydroisoquinoline-2-carbonyl)-4,4-dimethylpent-2-enenitrile | |
| C15 | 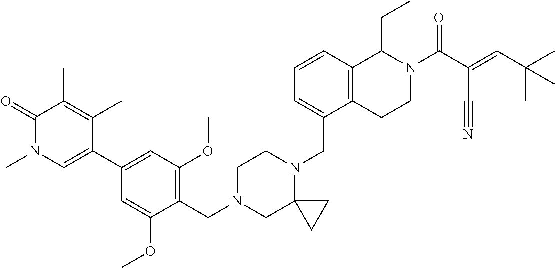 [SMILES: CCC1c2cccc(CN3CCN(Cc4c(OC)cc(-c5cn(C)c(=O)c(C)c5C)cc4OC)CC34CC4)c2CCN1C(=O)/C(C#N)=C/C(C)(C)C] | 706.2/15% |
| | (E/Z)-2-(5-((7-(2,6-dimethoxy-4-(1,4,5-trimethyl-6-oxo-1,6-dihydropyridin- | |
| | 3-yl)benzyl)-4,7-diazaspiro[2.5]octan-4-yl)methyl)-1-ethyl-1,2,3,4- | |
| | tetrahydroisoquinoline-2-carbonyl)-4,4-dimethylpent-2-enenitrile | |
| C16 | 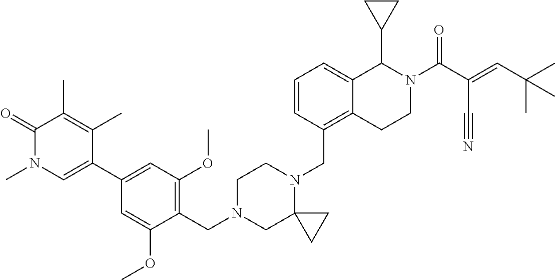 [SMILES: COc1cc(-c2cn(C)c(=O)c(C)c2C)cc(OC)c1CN1CCN(Cc2cccc3c2CCN(C(=O)/C(C#N)=C/C(C)(C)C)C3C2CC2)C2(CC2)C1] | 718.3/33% |
| | (E/Z)-2-(1-cyclopropyl-5-((7-(2,6-dimethoxy-4-(1,4,5-trimethyl-6-oxo-1,6- | |
| | dihydropyridin-3-yl)benzyl)-4,7-diazaspiro[2.5]octan-4-yl)methyl)-1,2,3,4- | |
| | tetrahydroisoquinoline-2-carbonyl)-4,4-dimethylpent-2-enenitrile | |
| C17 | 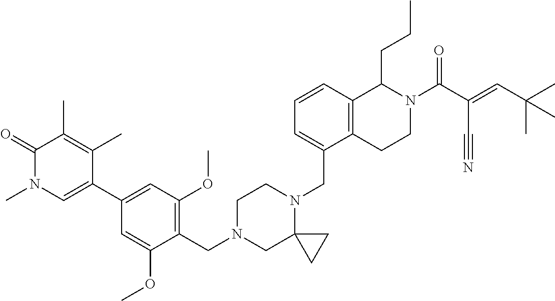 [SMILES: CCCC1c2cccc(CN3CCN(Cc4c(OC)cc(-c5cn(C)c(=O)c(C)c5C)cc4OC)CC34CC4)c2CCN1C(=O)/C(C#N)=C/C(C)(C)C] | 719.9/5% |
| | (E/Z)-2-(5-((7-(2,6-dimethoxy-4-(1,4,5-trimethyl-6-oxo-1,6-dihydropyridin- | |
| | 3-yl)benzyl)-4,7-diazaspiro[2.5]octan-4-yl)methyl)-1-propyl-1,2,3,4- | |
| | tetrahydroisoquinoline-2-carbonyl)-4,4-dimethylpent-2-enenitrile | |
| C18 | 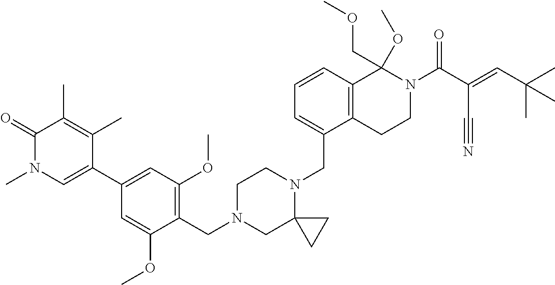 [SMILES: COCC1(OC)c2cccc(CN3CCN(Cc4c(OC)cc(-c5cn(C)c(=O)c(C)c5C)cc4OC)CC34CC4)c2CCN1C(=O)/C(C#N)=C/C(C)(C)C] | 752.4/3% |
| | (E/Z)-2-(5-((7-(2,6-dimethoxy-4-(1,4,5-trimethyl-6-oxo-1,6-dihydropyridin- | |
| | 3-yl)benzyl)-4,7-diazaspiro[2.5]octan-4-yl)methyl)-1-methoxy-1- | |
| | (methoxymethyl)-1,2,3,4-tetrahydroisoquinoline-2-carbonyl)-4,4- | |
| | dimethylpent-2-enenitrile | |

Table 8a: The following examples were prepared by general procedure 2 using the starting materials synthesized in Table 6a.

TABLE 8a

| | | LCMS m/z |
|---|---|---|
| | | [M + H]⁺/ |
| Name | Product Structure | Yield |
| 3-(5-((4-(2,6-dimethoxy-4-(2-methyl-1-oxo- 1,2-dihydro-2,7-naphthyridin-4- yl)benzyl)piperazin-1-yl)methyl)-1- methyl-3,4-dihydroisoquinolin-2(1H)- yl)-3-oxopropanenitrile | 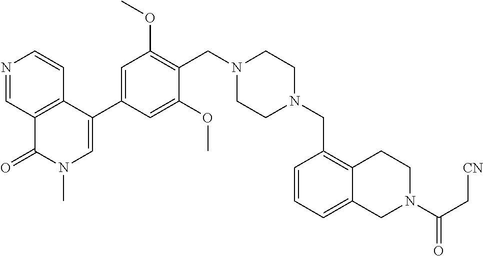 [SMILES: COc1cc(-c2cn(C)c(=O)c3cnccc23)cc(OC)c1CN1CCN(Cc2cccc3c2CCN(C(=O)CC#N)C3)CC1] | 621.2/58% |
| 3-(5-((4-(2,6-dimethoxy-4-(1,4,5- trimethyl-6-oxo-1,6-dihydropyridin-3- yl)benzyl)piperazin-1-yl)methyl)-1- methyl-3,4-dihydroisoquinolin-2(1H)- yl)-3-oxopropanenitrile | 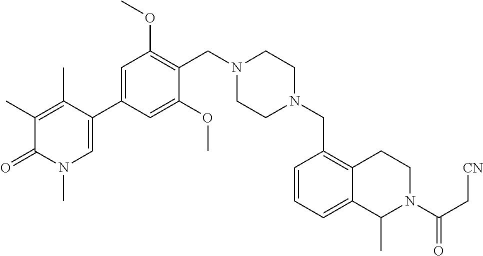 [SMILES: COc1cc(-c2cn(C)c(=O)c(C)c2C)cc(OC)c1CN1CCN(Cc2cccc3c2CCN(C(=O)CC#N)C3C)CC1] | 598.1/67% |
| 3-(5-((4-(2,6-dimethoxy-4-(2-methyl-1- oxo-1,2-dihydro-2,7-naphthyridin-4- yl)benzyl)-1,4-diazepan-1-yl)methyl)- 1-methyl-3,4-dihydroisoquinolin-2(1H)- yl)-3-oxopropanenitrile | 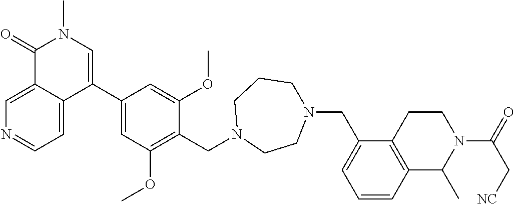 [SMILES: COc1cc(-c2cn(C)c(=O)c3cnccc23)cc(OC)c1CN1CCCN(Cc2cccc3c2CCN(C(=O)CC#N)C3C)CC1] | 635.4/47% |
| 3-[5-[[[2,6-dimethoxy-4-(2-methyl-1- oxo-2,7-naphthyridin-4-yl)phenyl] methyl-methyl-amino]methyl]-1- methyl-3,4-dihydro-1H-isoquinolin-2- yl]-3-oxo propanenitrile | 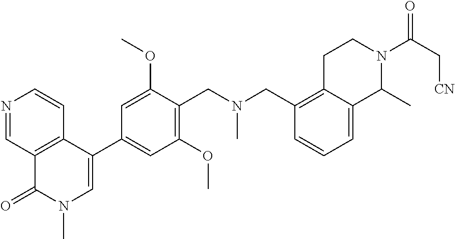 [SMILES: COc1cc(-c2cn(C)c(=O)c3cnccc23)cc(OC)c1CN(C)Cc1cccc2c1CCN(C(=O)CC#N)C2C] | 566.0/87% |
| 3-[6-[[[2,6-dimethoxy-4-(2-methyl-1- oxo-2,7-naphthyridin-4- yl)phenyl]methyl-methyl- amino]methyl]-1-methyl-3,4- dihydro-1H-isoquinolin-2-yl]-3- oxo-propanenitrile | 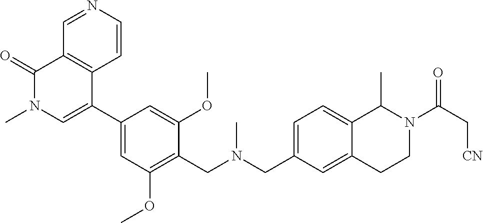 [SMILES: COc1cc(-c2cn(C)c(=O)c3cnccc23)cc(OC)c1CN(C)Cc1ccc2c(c1)CCN(C(=O)CC#N)C2C] | 566.0/87% |
| 3-[6-[[[2,6-dimethoxy-4-(2-methyl-1- oxo-2,7-naphthyridin-4- yl)phenyl]methyl-methyl- amino]methyl]-1-methyl-3,4-dihydro- 1H-isoquinolin-2-yl]-3- oxo-propanenitrile | 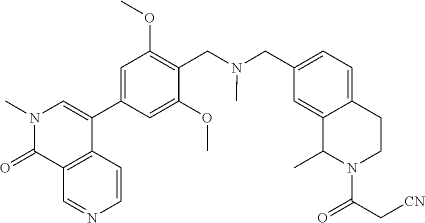 [SMILES: COc1cc(-c2cn(C)c(=O)c3cnccc23)cc(OC)c1CN(C)Cc1ccc2c(c1)C(C)N(C(=O)CC#N)CC2] | 566.0/88% |
| 3-(5-(((2-((2,6-dimethoxy-4-(2-methyl- 1-oxo-1,2-dihydro-2,7-naphthyridin-4- yl)benzyl)(methyl)amino)ethyl)(methyl) amino)methyl)-1-methyl-3,4- dihydroisoquinolin-2(1H)-yl)-3- oxopropanenitrile | 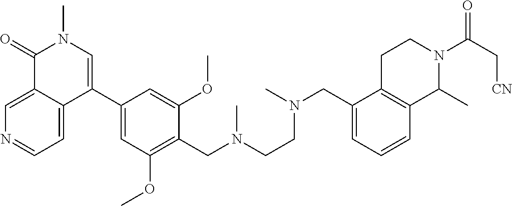 [SMILES: COc1cc(-c2cn(C)c(=O)c3cnccc23)cc(OC)c1CN(C)CCN(C)Cc1cccc2c1CCN(C(=O)CC#N)C2C] | 623.4/25% |
| 3-(5-((5-(2,6-dimethoxy-4-(2-methyl-1- oxo-1,2-dihydro-2,7-naphthyridin-4- yl)benzyl)-2,5- diazabicyclo[2.2.1]heptan-2- yl)methyl)-1-methyl-3,4- dihydroisoquinolin-2(1H)-yl)-3- oxopropanenitrile | 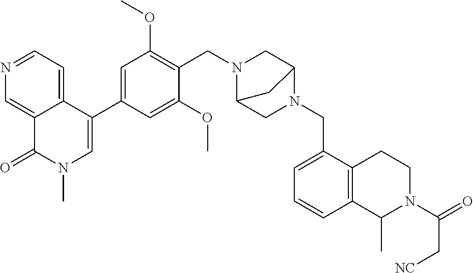 [SMILES: COc1cc(-c2cn(C)c(=O)c3cnccc23)cc(OC)c1CN1CC2CC1CN2Cc1cccc2c1CCN(C(=O)CC#N)C2C] | 633.0/87% |
| 3-[5-[[6-[[2,6-dimethoxy-4-(2-methyl-1- oxo-2,7-naphthyridin-4- yl)phenyl]methyl]-3,6- diazabicyclo[3.1.1]heptan-3-yl]methyl]- 1-methyl-3,4-dihydro-1H-isoquinolin-2- yl]-3-oxo-propanenitrile | 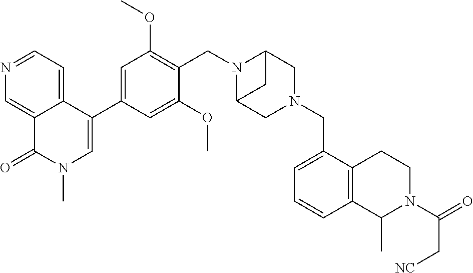 [SMILES: COc1cc(-c2cn(C)c(=O)c3cnccc23)cc(OC)c1CN1C2CC1CN(Cc1cccc3c1CCN(C(=O)CC#N)C3C)C2] | 633.0/76% |
| 3-(5-((3-(2,6-dimethoxy-4-(2-methyl-1- oxo-1,2-dihydro-2,7-naphthyridin-4- yl)benzyl)-3,6- diazabicyclo[3.1.1]heptan-6- yl)methyl)-1-methyl-3,4- dihydroisoquinolin-2(1H)-yl)-3- oxopropanenitrile | 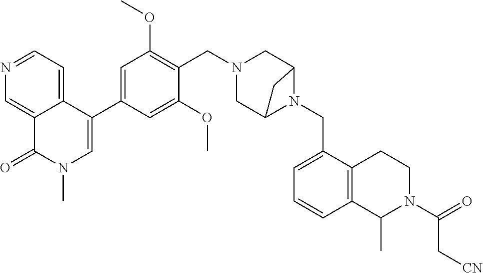 [SMILES: COc1cc(-c2cn(C)c(=O)c3cnccc23)cc(OC)c1CN1CC2CC(C1)N2Cc1cccc2c1CCN(C(=O)CC#N)C2C] | 633.1/32% |
| 3-(5-((3-(2,6-dimethoxy-4-(2-methyl-1- oxo-1,2-dihydro-2,7-naphthyridin-4- yl)benzyl)-3,8- diazabicyclo[3.2.1]octan-8-yl)methyl)- 1-methyl-3,4-dihydroisoquinolin-2(1H)- yl)-3-oxopropanenitrile | 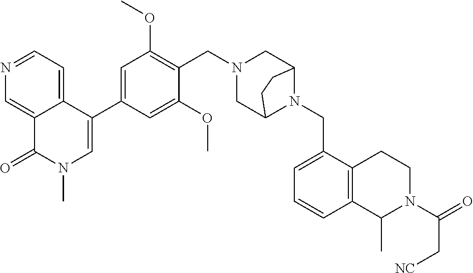 [SMILES: COc1cc(-c2cn(C)c(=O)c3cnccc23)cc(OC)c1CN1CC2CCC(C1)N2Cc1cccc2c1CCN(C(=O)CC#N)C2C] | 647.1/31% |
| 3-(5-((4-(4-(1,5-dimethyl-6-oxo-1,6- dihydropyridin-3-yl)-2,6- dimethoxybenzyl)piperazin-1- yl)methyl)-1-methyl-3,4- dihydroisoquinolin-2(1H)-yl)-3- oxopropanenitrile | 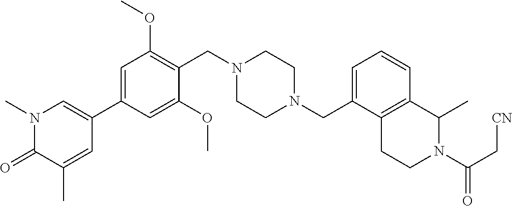 [SMILES: COc1cc(-c2cc(C)c(=O)n(C)c2)cc(OC)c1CN1CCN(Cc2cccc3c2CCN(C(=O)CC#N)C3C)CC1] | 584.3/74% |
| 3-(5-((4-(2,6-dimethoxy-4-(1,4,5- trimethyl-6-oxo-1,6-dihydropyridin-3- yl)benzyl)-1,4-diazepan-1-yl)methyl)- 1-methyl-3,4-dihydroisoquinolin-2(1H)- yl)-3-oxopropanenitrile | 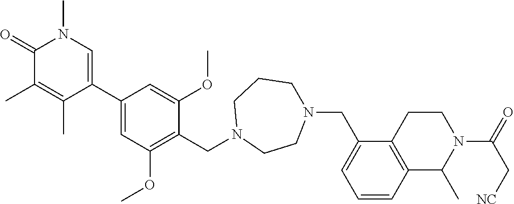 [SMILES: COc1cc(-c2cn(C)c(=O)c(C)c2C)cc(OC)c1CN1CCCN(Cc2cccc3c2CCN(C(=O)CC#N)C3C)CC1] | 612.3/83% |
| 3-(5-((5-(2,6-dimethoxy-4-(1,4,5- trimethyl-6-oxo-1,6-dihydropyridin-3- yl)benzyl)-2,5- diazabicyclo[2.2.1]heptan-2- yl)methyl)-1-methyl-3,4- dihydroisoquinolin-2(1H)- yl)-3-oxopropanenitrile | 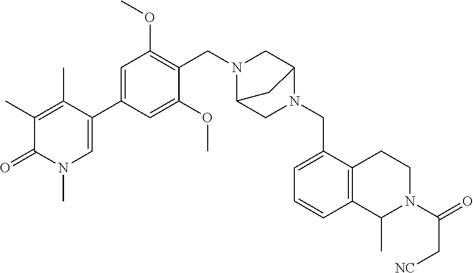 [SMILES: COc1cc(-c2cn(C)c(=O)c(C)c2C)cc(OC)c1CN1CC2CC1CN2Cc1cccc2c1CCN(C(=O)CC#N)C2C] | 610.5/45% |
| 3-(5-((3-(2,6-dimethoxy-4-(1,4,5- trimethyl-6-oxo-1,6-dihydropyridin-3- yl)benzyl)-3,6- diazabicyclo[3.1.1]heptan-6-yl) methyl)-1-methyl-3,4- dihydroisoquinolin-2(1H)-yl)-3- oxopropanenitrile | 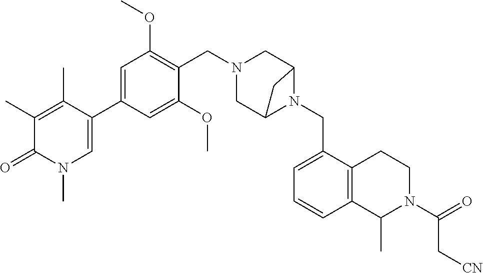 [SMILES: COc1cc(-c2cn(C)c(=O)c(C)c2C)cc(OC)c1CN1CC2CC(C1)N2Cc1cccc2c1CCN(C(=O)CC#N)C2C] | 610.5/41% |
| 3-(5-((4-(2,6-dimethoxy-4-(6-methyl-7- oxo-6,7-dihydro-1H-pyrazolo[3,4- c]pyridin-4-yl)benzyl)-4,7- diazaspiro[2.5]octan-7-yl)methyl)-1- methyl-3,4-dihydroisoquinolin-2(1H)- yl)-3-oxopropanenitrile | 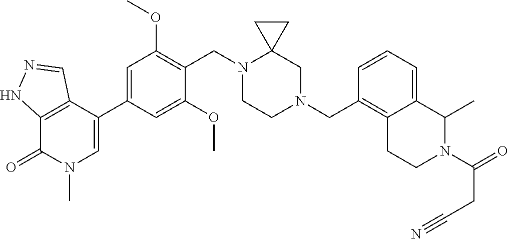 [SMILES: COc1cc(-c2cn(C)c(=O)c3[nH]ncc23)cc(OC)c1CN1CCN(Cc2cccc3c2CCN(C(=O)CC#N)C3C)CC12CC2] | 636.4/25% |
| 3-(5-((7-(2,6-dimethoxy-4-(1,4,5- trimethyl-6-oxo-1,6-dihydropyridin-3- yl)benzyl)-4,7-diazaspiro[2.5]octan-4- yl)methyl)-1-methyl-3,4- dihydroisoquinolin-2(1H)- yl)-3-oxopropanenitrile | 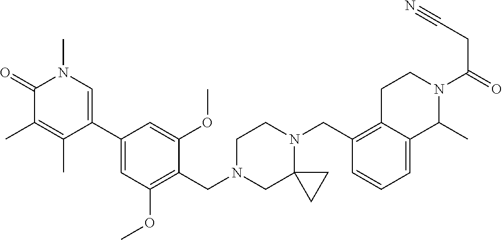 [SMILES: COc1cc(-c2cn(C)c(=O)c(C)c2C)cc(OC)c1CN1CCN(Cc2cccc3c2CCN(C(=O)CC#N)C3C)C2(CC2)C1] | 624.4/65% |
| 3-(5-((4-(2,6-dimethoxy-4-(1,4,5- trimethyl-6-oxo-1,6-dihydropyridin-3- yl)benzoyl)piperazin-1-yl)methyl)-1- methyl-3,4-dihydroisoquinolin-2(1H)- yl)-3-oxopropanenitrile | 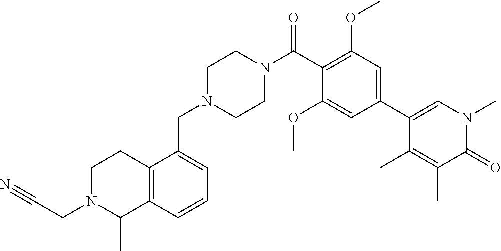 [SMILES: COc1cc(-c2cn(C)c(=O)c(C)c2C)cc(OC)c1C(=O)N1CCN(Cc2cccc3c2CCN(CC#N)C3C)CC1] | 612.3/83% |
| 3-(5-((7-(2,6-dimethoxy-4-(6-methyl-7- oxo-6,7-dihydro-1H-pyrazolo[3,4- c]pyridin-4-yl)benzyl)-4,7- diazaspiro[2.5]octan-4-yl)methyl)-1- methyl-3,4-dihydroisoquinolin-2(1H)- yl)-3-oxopropanenitrile | 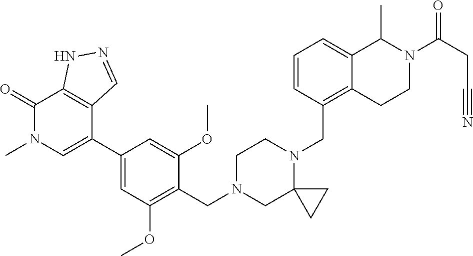 [SMILES: COc1cc(-c2cn(C)c(=O)c3[nH]ncc23)cc(OC)c1CN1CCN(Cc2cccc3c2CCN(C(=O)CC#N)C3C)C2(CC2)C1] | 612.3/41% |
| 3-(5-((7-(2,6-dimethoxy-4-(5-methyl-4- oxo-4,5-dihydrothieno[3,2-c]pyridin-7- yl)benzyl)-4,7-diazaspiro[2.5]octan-4- yl)methyl)-1-methyl-3,4- dihydroisoquinolin-2(1H)- yl)-3-oxopropanenitrile | 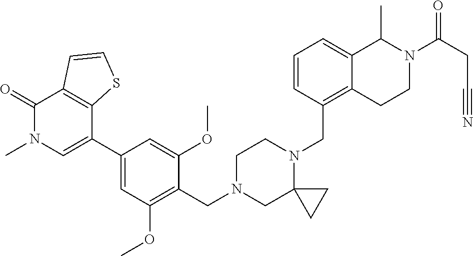 [SMILES: COc1cc(-c2cn(C)c(=O)c3ccsc23)cc(OC)c1CN1CCN(Cc2cccc3c2CCN(C(=O)CC#N)C3C)C2(CC2)C1] | 652.3/45% |
| 3-(5-((3-(2,6-dimethoxy-4-(1,4,5- trimethyl-6-oxo-1,6-dihydropyridin-3- yl)benzyl)-3,8- diazabicyclo[3.2.1]octan-8-yl)methyl)- 1-methyl-3,4-dihydroisoquinolin-2(1H)- yl)-3-oxopropanenitrile | 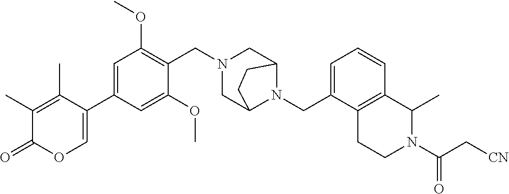 [SMILES: COc1cc(-c2coc(=O)c(C)c2C)cc(OC)c1CN1CC2CCC(C1)N2Cc1cccc2c1CCN(C(=O)CC#N)C2C] | 624.4/45% |
| 3-(5-((3-(2,6-dimethoxy-4-(1,4,5- trimethyl-6-oxo-1,6-dihydropyridin-3- yl)benzyl)-3,6- diazabicyclo[3.1.1]heptan-6- yl)methyl)-1-methyl-3,4- dihydroisoquinolin-2(1H)- yl)-3-oxopropanenitrile | 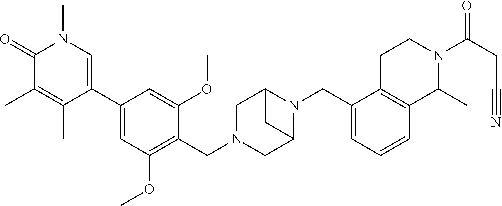 [SMILES: COc1cc(-c2cn(C)c(=O)c(C)c2C)cc(OC)c1CN1CC2CC(C1)N2Cc1cccc2c1CCN(C(=O)CC#N)C2C] | 610.2/29% |
| 3-(5-((4-(2,6-dimethoxy-4-(6-methyl-7- oxo-6,7-dihydro-1H-pyrazolo[3,4- c]pyridin-4-yl)benzyl)piperazin-1- yl)methyl)-1-methyl-3,4- dihydroisoquinolin-2(1H)- yl)-3-oxopropanenitrile | 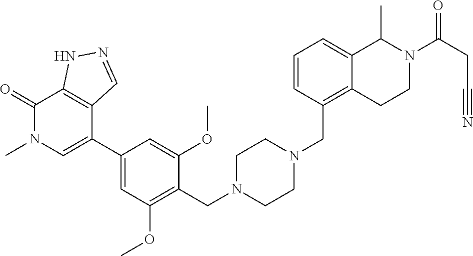 [SMILES: COc1cc(-c2cn(C)c(=O)c3[nH]ncc23)cc(OC)c1CN1CCN(Cc2cccc3c2CCN(C(=O)CC#N)C3C)CC1] | 610.4/50% |
| 3-(5-((4-(2,6-dimethoxy-4-(1,4,5- trimethyl-6-oxo-1,6-dihydropyridin-3- yl)benzyl)-2,2-dimethylpiperazin-1- yl)methyl)-1-methyl-3,4- dihydroisoquinolin-2(1H)- yl)-3-oxopropanenitrile | 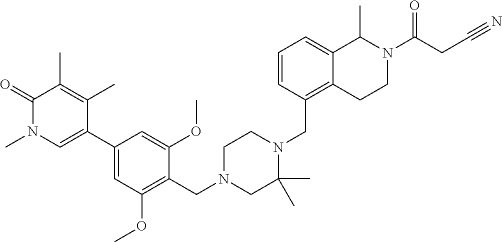 [SMILES: COc1cc(-c2cn(C)c(=O)c(C)c2C)cc(OC)c1CN1CCN(Cc2cccc3c2CCN(C(=O)CC#N)C3C)C(C)(C)C1] | 626.4/32% |
| 3-(5-(((2S,6S)-4-(2,6-dimethoxy-4- (1,4,5-trimethyl-6-oxo-1,6- dihydropyridin-3-yl)benzyl)-2,6- dimethylpiperazin-1-yl)methyl)-1- methyl-3,4-dihydroisoquinolin-2(1H)- yl)-3-oxopropanenitrile | 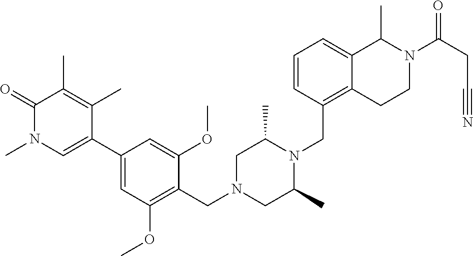 [SMILES: COc1cc(-c2cn(C)c(=O)c(C)c2C)cc(OC)c1CN1C[C@H](C)N(Cc2cccc3c2CCN(C(=O)CC#N)C3C)[C@@H](C)C1] | 626.4/39% |
| 3-(7-(((2,6-dimethoxy-4-(1,4,5- trimethyl-6-oxo-1,6-dihydropyridin-3- yl)benzyl(methyl)amino)methyl)-1- methyl-3,4-dihydroisoquinolin-2(1H)- yl)-3-oxopropanenitrile | 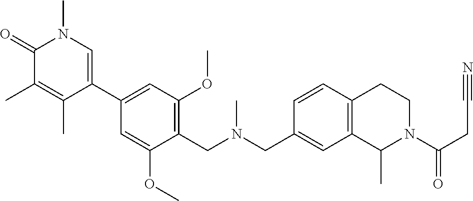 [SMILES: COc1cc(-c2cn(C)c(=O)c(C)c2C)cc(OC)c1CN(C)Cc1ccc2c(c1)C(C)N(C(=O)CC#N)CC2] | 543.2/94% |
| 3-(5-(((2,6-dimethoxy-4-(1,4,5- trimethyl-6-oxo-1,6-dihydropyridin-3- yl)benzyl)(methyl)amino)methyl)-1- methyl-3,4-dihydroisoquinolin-2(1H)- yl)-3-oxopropanenitrile | 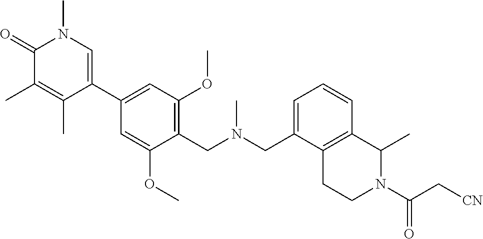 [SMILES: COc1cc(-c2cn(C)c(=O)c(C)c2C)cc(OC)c1CN(C)Cc1cccc2c1CCN(C(=O)CC#N)C2C] | 543.2/46% |
| 3-(5-(((2R,5S)-4-(2,6-dimethoxy-4- (1,4,5-trimethyl-6-oxo-1,6- dihydropyridin-3-yl)benzyl)-2,5- dimethylpiperazin-1-yl)methyl)-1- methyl-3,4-dihydroisoquinolin-2(1H)- yl)-3-oxopropanenitrile | 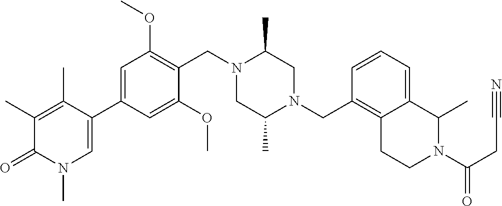 [SMILES: COc1cc(-c2cn(C)c(=O)c(C)c2C)cc(OC)c1CN1C[C@@H](C)N(Cc2cccc3c2CCN(C(=O)CC#N)C3C)C[C@@H]1C] | 626.4/66% |
| 3-(5-((4-(2,6-dimethoxy-4-(1,4,5- trimethyl-6-oxo-1,6-dihydropyridin-3- yl)benzyl)-2-isopropylpiperazin-1- yl)methyl)-1-methyl-3,4- dihydroisoquinolin-2(1H)- yl)-3-oxopropanenitrile | 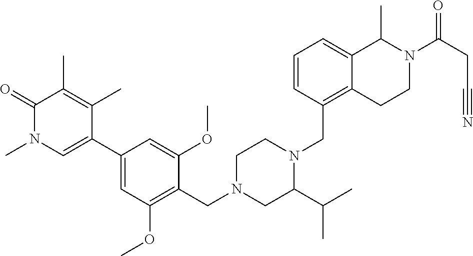 [SMILES: COc1cc(-c2cn(C)c(=O)c(C)c2C)cc(OC)c1CN1CCN(Cc2cccc3c2CCN(C(=O)CC#N)C3C)C(C(C)C)C1] | 640.4/80% |
| 3-(7-((7-(2,6-dimethoxy-4-(1,4,5- trimethyl-6-oxo-1,6-dihydropyridin-3- yl)benzyl)-4,7-diazaspiro[2.5]octan-4- yl)methyl)-1-methyl-3,4- dihydroisoquinolin-2(1H)- yl)-3-oxopropanenitrile | 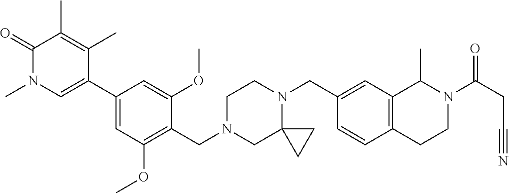 [SMILES: COc1cc(-c2cn(C)c(=O)c(C)c2C)cc(OC)c1CN1CCN(Cc2ccc3c(c2)C(C)N(C(=O)CC#N)CC3)C2(CC2)C1] | 624.3/60% |
| 3-(5-((4-(2,6-dimethoxy-4-(1,4,5- trimethyl-6-oxo-1,6-dihydropyridin-3- yl)benzyl)-4,7-diazaspiro[2.5]octan-7- yl)methyl)-1-methyl-3,4- dihydroisoquinolin-2(1H)- yl)-3-oxopropanenitrile | 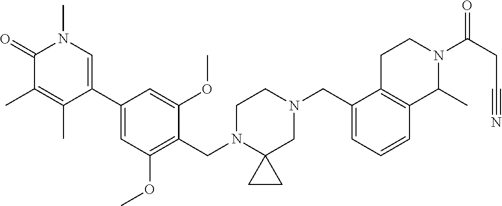 [SMILES: COc1cc(-c2cn(C)c(=O)c(C)c2C)cc(OC)c1CN1CCN(Cc2cccc3c2CCN(C(=O)CC#N)C3C)CC12CC2] | 624.3/44% |
| 3-(5-((6-(2,6-dimethoxy-4-(1,4,5- trimethyl-6-oxo-1,6-dihydropyridin-3- yl)benzyl)-3,6- diazabicyclo[3.1.1]heptan-3- yl)methyl)-1-methyl-3,4- dihydroisoquinolin-2(1H)- yl)-3-oxopropanenitrile | 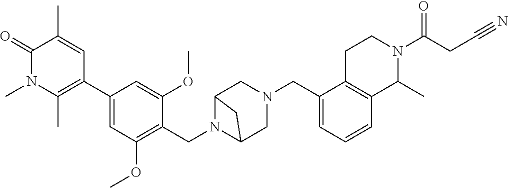 [SMILES: COc1cc(-c2cc(C)c(=O)n(C)c2C)cc(OC)c1CN1C2CC1CN(Cc1cccc3c1CCN(C(=O)CC#N)C3C)C2] | 610.3/30% |
| 3-(4-(2,6-dimethoxy-4-(1,4,5-trimethyl- 6-oxo-1,6-dihydropyridin-3- yl)benzyl)piperazin-1-yl)-3- oxopropanenitrile | 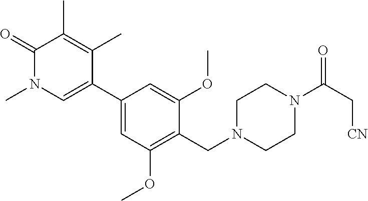 [SMILES: COc1cc(-c2cn(C)c(=O)c(C)c2C)cc(OC)c1CN1CCN(C(=O)CC#N)CC1] | 439.3/40% |
| 3-(5-(2,6-dimethoxy-4-(1,4,5-trimethyl- 6-oxo-1,6-dihydropyridin-3-yl)benzyl)- 2,5-diazabicyclo[2.2.1]heptan-2-yl)-3- oxopropanenitrile | 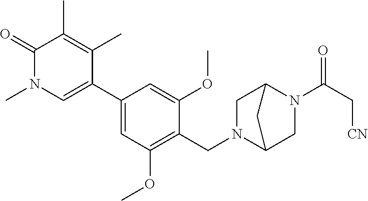 [SMILES: COc1cc(-c2cn(C)c(=O)c(C)c2C)cc(OC)c1CN1CC2CC1CN2C(=O)CC#N] | 451.4/62% |
| 3-(3-(2,6-dimethoxy-4-(1,4,5-trimethyl- 6-oxo-1,6-dihydropyridin-3-yl)benzyl)- 3,6-diazabicyclo[3.1.1]heptan-6-yl)-3- oxopropanenitrile | 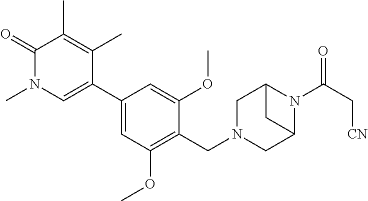 [SMILES: COc1cc(-c2cn(C)c(=O)c(C)c2C)cc(OC)c1CN1CC2CC(C1)N2C(=O)CC#N] | 451.5/60% |
| 3-(6-(2,6-dimethoxy-4-(1,4,5-trimethyl- 6-oxo-1,6-dihydropyridin-3-yl)benzyl)- 3,6-diazabicyclo[3.1.1]heptan-3-yl)-3- oxopropanenitrile | 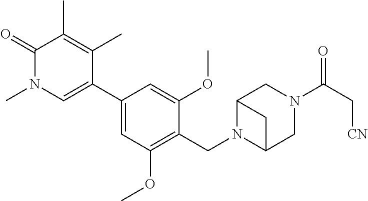 [SMILES: COc1cc(-c2cn(C)c(=O)c(C)c2C)cc(OC)c1CN1C2CC1CN(C(=O)CC#N)C2] | 451.4/49% |
| 3-(3-(2,6-dimethoxy-4-(1,4,5-trimethyl- 6-oxo-1,6-dihydropyridin-3-yl)benzyl)- 3,8-diazabicyclo[3.2.1]octan-8-yl)-3- oxopropanenitrile | 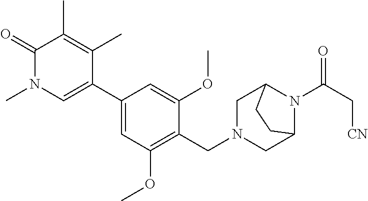 [SMILES: COc1cc(-c2cn(C)c(=O)c(C)c2C)cc(OC)c1CN1CC2CCC(C1)N2C(=O)CC#N] | 465.2/11% |
| 3-(4-((2,6-dimethoxy-4-(1,4,5- trimethyl-6-oxo-1,6-dihydropyridin-3- yl)benzyl)(methyl)amino)piperidin-1- yl)-3-oxopropanenitrile | 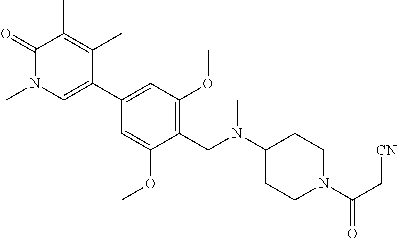 [SMILES: COc1cc(-c2cn(C)c(=O)c(C)c2C)cc(OC)c1CN(C)C1CCN(C(=O)CC#N)CC1] | 467.2/68% |
| 3-(9-(2,6-dimethoxy-4-(1,4,5-trimethyl- 6-oxo-1,6-dihydropyridin-3-yl)benzyl)- 3,9-diazaspiro[5.5]undecan-3-yl)-3- oxopropanenitrile | 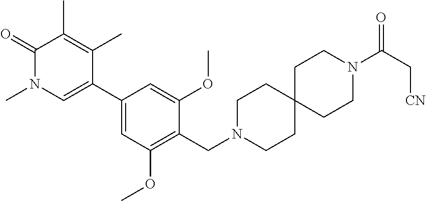 [SMILES: COc1cc(-c2cn(C)c(=O)c(C)c2C)cc(OC)c1CN1CCC2(CC1)CCN(C(=O)CC#N)CC2] | 507.4/45% |
| 2-cyano-N-(2,6-dimethoxy-4-(1,4,5- trimethyl-6-oxo-1,6-dihydropyridin-3- yl)benzyl)-N-methylacetamide | 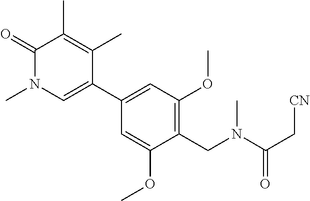 [SMILES: COc1cc(-c2cn(C)c(=O)c(C)c2C)cc(OC)c1CN(C)C(=O)CC#N] | 384.5/32% |
| 3-(5-((7-(2,6-dimethoxy-4-(1,4,5- trimethyl-6-oxo-1,6-dihydropyridin-3- yl)benzyl)-4,7-diazaspiro[2.5]octan-4- yl)methyl)-1-methyl-3,4- dihydroisoquinolin-2(1H)-yl)-3- oxopropanenitrile | 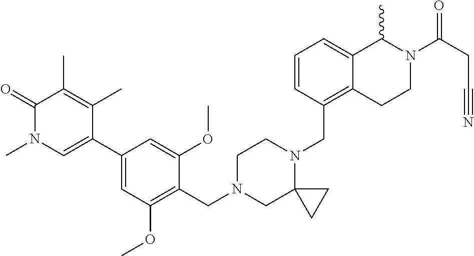 [SMILES: COc1cc(-c2cn(C)c(=O)c(C)c2C)cc(OC)c1CN1CCN(Cc2cccc3c2CCN(C(=O)CC#N)C3C)C2(CC2)C1] | 624.4/89% |
| | single enantiomer of unknown | |
| | absolute configuration | |
| | (using peak 1 of tert-butyl 5-bromo-1- | |
| | methyl-3,4-dihydroisoquinoline-2(1H)- | |
| | carboxylate | |
| 3-(5-((7-(2,6-dimethoxy-4-(1,4,5- trimethyl-6-oxo-1,6-dihydropyridin-3- yl)benzyl)-4,7-diazaspiro[2.5]octan-4- yl)methyl)-1-methyl-3,4- dihydroisoquinolin-2(1H)-yl)-3- oxopropanenitrile | 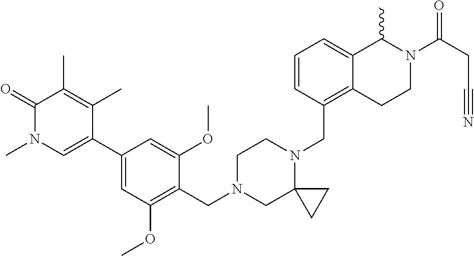 [SMILES: COc1cc(-c2cn(C)c(=O)c(C)c2C)cc(OC)c1CN1CCN(Cc2cccc3c2CCN(C(=O)CC#N)C3C)C2(CC2)C1] | 624.4/90% |
| | single enantiomer of unknown | |
| | absolute configuration | |
| | (using peak 2 of tert-butyl 5-bromo-1- | |
| | methyl-3,4-dihydroisoquinoline-2(1H)- | |
| | carboxylate) | |
| 3-(5-((4-((2,6-dimethoxy-4-(1,4,5- trimethyl-6-oxo-1,6-dihydropyridin-3- yl)benzyl)-1,4-diazepan-1-yl)methyl)- 1-methyl-3,4-dihydroisoquinolin-2(1H)- yl)-3-oxopropanenitrile | 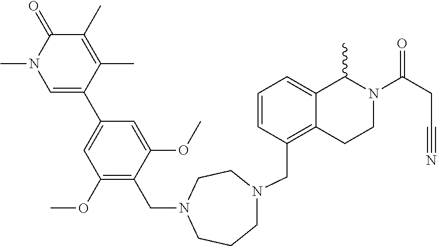 [SMILES: COc1cc(-c2cn(C)c(=O)c(C)c2C)cc(OC)c1CN1CCCN(Cc2cccc3c2CCN(C(=O)CC#N)C3C)CC1] | 612.3/78% |
| | single enantiomer of unknown absolute configuration | |
| | (using peak 2 of tert-butyl 5-bromo-1-methyl-3,4- | |
| | dihydroisoquinoline-2(1H)-carboxylate) | |
| 3-(5-((4-(2,6-dimethoxy-4-(1,4,5- trimethyl-6-oxo-1,6-dihydropyridin-3- yl)benzyl)piperazin-1-yl)methyl)-1- isopropyl-3,4-dihydroisoquinolin- 2(1H)-yl)-3-oxopropanenitrile | 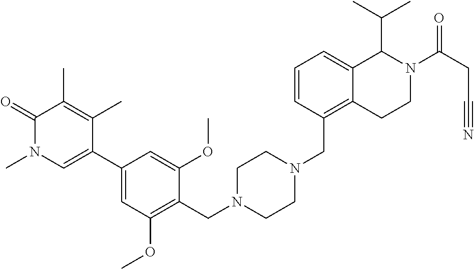 [SMILES: COc1cc(-c2cn(C)c(=O)c(C)c2C)cc(OC)c1CN1CCN(Cc2cccc3c2CCN(C(=O)CC#N)C3C(C)C)CC1] | 626.5/50% |
| 3-(5-((7-(2,6-dimethoxy-4-(1,4,5- trimethyl-6-oxo-1,6-dihydropyridin-3- yl)benzyl)-4,7-diazaspiro[2.5]octan-4- yl)methyl)-1-isopropyl-3,4- dihydroisoquinolin-2(1H)-yl)-3- oxopropanenitrile | 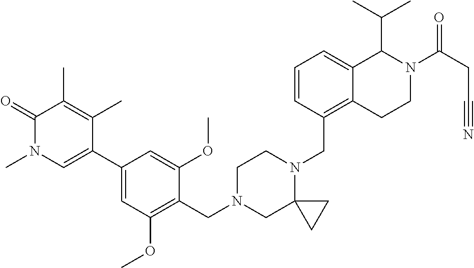 [SMILES: COc1cc(-c2cn(C)c(=O)c(C)c2C)cc(OC)c1CN1CCN(Cc2cccc3c2CCN(C(=O)CC#N)C3C(C)C)C2(CC2)C1] | 652.4/54% |
| 3-(5-((4-(2,6-dimethoxy-4-(1,4,5- trimethyl-6-oxo-1,6-dihydropyridin-3- yl)benzyl)-4,7-diazaspiro[2.5]octan-7- yl)methyl)-1-isopropyl-3,4- dihydroisoquinolin-2(1H)-yl)-3- oxopropanenitrile | 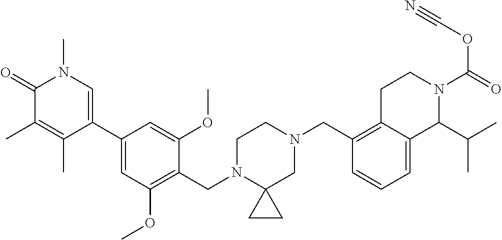 [SMILES: COc1cc(-c2cn(C)c(=O)c(C)c2C)cc(OC)c1CN1CCN(Cc2cccc3c2CCN(C(=O)OC#N)C3C(C)C)CC12CC2] | 652.3/71% |
| 3-(5-(((S)-4-(2,6-dimethoxy-4-(1,4,5- trimethyl-6-oxo-1,6-dihydropyridin-3- yl)benzyl)-2-methylpiperazin-1- yl)methyl)-1-isopropyl-3,4- dihydroisoquinolin-2(1H)-yl)-3- oxopropanenitrile | 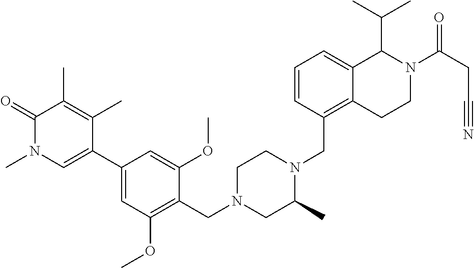 [SMILES: COc1cc(-c2cn(C)c(=O)c(C)c2C)cc(OC)c1CN1CCN(Cc2cccc3c2CCN(C(=O)CC#N)C3C(C)C)[C@@H](C)C1] | 640.5/52% |
| 3-(5-((4-(2,6-dimethoxy-4-(1,4,5- trimethyl-6-oxo-1,6-dihydropyridin-3- yl)benzyl)-1,4-diazepan-1-yl)methyl)- 1-isopropyl-3,4-dihydroisoquinolin- 2(1H)-yl)-3-oxopropanenitrile | 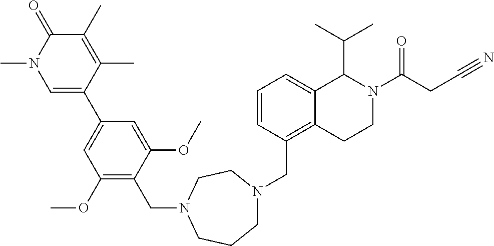 [SMILES: COc1cc(-c2cn(C)c(=O)c(C)c2C)cc(OC)c1CN1CCCN(Cc2cccc3c2CCN(C(=O)CC#N)C3C(C)C)CC1] | 640.2/36% |
| 3-(5-((7-(2,6-dimethoxy-4-(1,4,5- trimethyl-6-oxo-1,6-dihydropyridin-3- yl)benzyl)-4,7-diazaspiro[2.5]octan-4- yl)methyl)-1-ethyl-3,4- dihydroisoquinolin-2(1H)-yl)-3- oxopropanenitrile | 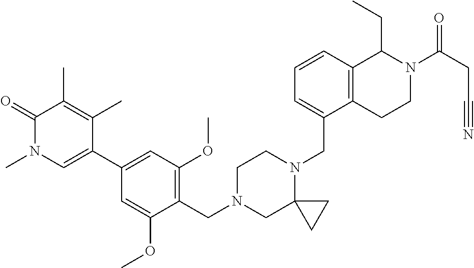 [SMILES: CCC1c2cccc(CN3CCN(Cc4c(OC)cc(-c5cn(C)c(=O)c(C)c5C)cc4OC)CC34CC4)c2CCN1C(=O)CC#N] | 638.2/46% |

Table 9a: The following analogues were made according to the relevant procedures as noted in the table below from compounds shown in Table 8a.

| | | | LCMS |
|---|---|---|---|
| | | | m/z |
| | | | [M + H]⁺/ |
| Cmpd | Product Structure/Name | Procedure | Yield |
| A46 | 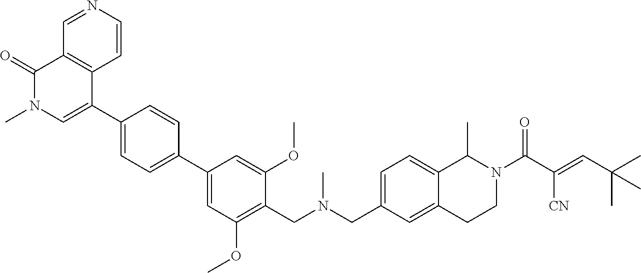 [SMILES: COc1cc(-c2ccc(-c3cn(C)c(=O)c4cnccc34)cc2)cc(OC)c1CN(C)Cc1ccc2c(c1)CCN(C(=O)/C(C#N)=C/C(C)(C)C)C2C] | 4 | 634.5/ 21% |
| | (E/Z)-2-[6-[[[2,6-dimethoxy-4-(2-methyl-1-oxo-2,7- | | |
| | naphthyridin-4-yl)phenyl]methyl-methyl-aminojmethyl]-1- | | |
| | methyl-3,4-dihydro-1H-isoquinoline-2-carbonyl]-4,4- | | |
| | dimethyl-pent-2-enenitrile | | |
| A47 | 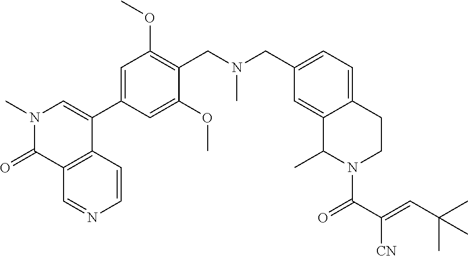 [SMILES: COc1cc(-c2cn(C)c(=O)c3cnccc23)cc(OC)c1CN(C)Cc1ccc2c(c1)C(C)N(C(=O)/C(C#N)=C/C(C)(C)C)CC2] | 4 | 634.4/ 21% |
| | (E/Z)-2-[7-[[2,6-dimethoxy-4-(2-methyl-1-oxo-2,7- | | |
| | naphthyridin-4-yl)phenylmethyl-methyl-amino]methyl]-1- | | |
| | methyl-3,4-dihydro-1H-isoquinoline-2-carbonyl]-4,4- | | |
| | dimethyl-pent-2-enenitrile | | |
| A31 | 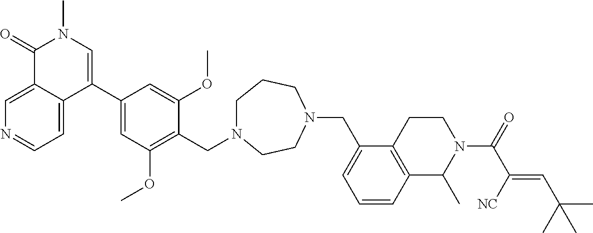 [SMILES: COc1cc(-c2cn(C)c(=O)c3cnccc23)cc(OC)c1CN1CCCN(Cc2cccc3c2CCN(C(=O)/C(C#N)=C/C(C)(C)C)C3C)CC1] | 4 | 703.3/ 16% |
| | (E/Z)-2-(5-((4-(2,6-dimethoxy-4-(2-methyl-1-oxo-1,2- | | |
| | dihydro-2,7-naphthyridin-4-yl) benzyl)-1,4-diazepan-1-yl) | | |
| | methyl)-1-methyl-1,2,3,4-tetrahydroisoquinoline-2- | | |
| | carbonyl)-4,4-dimethylpent-2-enenitrile | | |
| A32 | 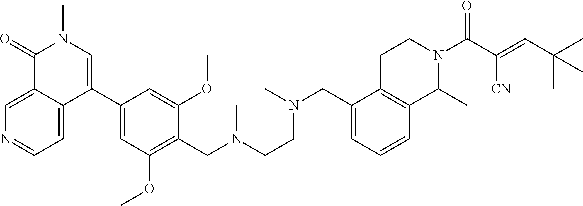 [SMILES: COc1cc(-c2cn(C)c(=O)c3cnccc23)cc(OC)c1CN(C)CCN(C)Cc1cccc2c1CCN(C(=O)/C(C#N)=C/C(C)(C)C)C2C] | 4 | 691.4/ 30% |
| | (E/Z)-2-(5-(((2-((2,6-dimethoxy-4-(2-methyl-1-oxo-1,2- | | |
| | dihydro-2,7-naphthyridin-4- | | |
| | yl)benzyl)(methyl)amino)ethyl)(methyl)amino)methyl)-1- | | |
| | methyl-1,2,3,4-tetrahydroisoquinoline-2-carbonyl)-4,4- | | |
| | dimethylpent-2-enenitrile | | |
| A39 | 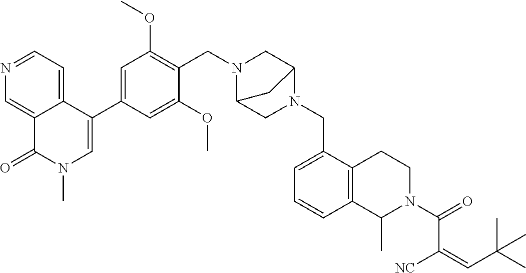 [SMILES: COc1cc(-c2cn(C)c(=O)c3cnccc23)cc(OC)c1CN1CC2CC1CN2Cc1cccc2c1CCN(C(=O)/C(C#N)=C\C(C)(C)C)C2C] | 4 | 701.2/ 12% |
| | (E/Z)-2-(5-((5-(2,6-dimethoxy-4-(2-methyl-1-oxo-1,2- | | |
| | dihydro-2,7-naphthyridin-4-yl)benzyl)-2,5- | | |
| | diazabicyclo[2.2.1]heptan-2-yl)methyl)-1-methyl-1,2,3,4- | | |
| | tetrahydroisoquinoline-2-carbonyl)-4,4-dimethylpent-2- | | |
| | enenitrile | | |
| A40 | 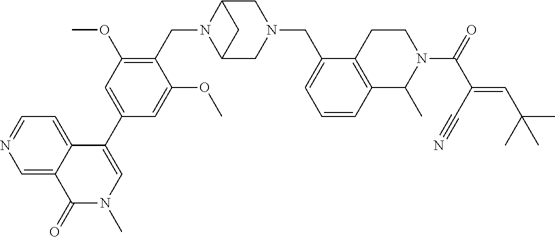 [SMILES: COc1cc(-c2cn(C)c(=O)c3cnccc23)cc(OC)c1CN1C2CC1CN(Cc1cccc3c1CCN(C(=O)/C(C#N)=C/C(C)(C)C)C3C)C2] | 4 | 701.0/ 12% |
| | (E/Z)-2-[5-[6-[2,6-dimethoxy-4-(2-methyl-1-oxo-2,7- | | |
| | naphthyridin-4-yl)phenyl]methyl]-3,6- | | |
| | diazabicyclo[3.1.1]heptan-3-yl]methyl]-1-methyl-3,4- | | |
| | dihydro-1H-isoquinoline-2-carbonyl]-4,4-dimethyl-pent-2- | | |
| | enenitrile | | |
| A41 | 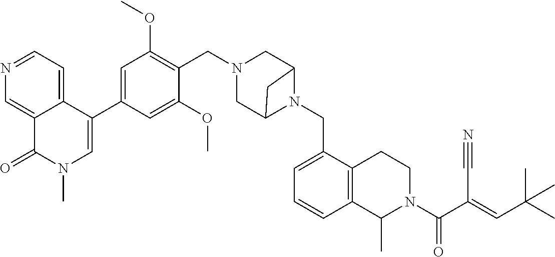 [SMILES: COc1cc(-c2cn(C)c(=O)c3cnccc23)cc(OC)c1CN1CC2CC(C1)N2Cc1cccc2c1CCN(C(=O)/C(C#N)=C/C(C)(C)C)C2C] | 4 | 701.2/ 3% |
| | (E/Z)-2-(5-((3-(2,6-dimethoxy-4-(2-methyl-1-oxo-1,2- | | |
| | dihydro-2,7-naphthyridin-4-yl)benzyl)-3,6- | | |
| | diazabicyclo[3.1.1]heptan-6-yl)methyl)-1-methyl-1,2,3,4- | | |
| | tetrahydroisoquinoline-2-carbonyl)-4,4-dimethylpent-2- | | |
| | enenitrile | | |
| A48 | 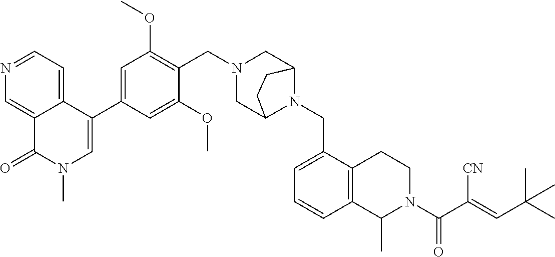 [SMILES: COc1cc(-c2cn(C)c(=O)c3cnccc23)cc(OC)c1CN1CC2CCC(C1)N2Cc1cccc2c1CCN(C(=O)/C(C#N)=C/C(C)(C)C)C2C] | 4 | 716.6/ 26% |
| | (E/Z)-2-(5-((3-(2,6-dimethoxy-4-(2-methyl-1-oxo-1,2- | | |
| | dihydro-2,7-naphthyridin-4-yl)benzyl)-3,8- | | |
| | diazabicyclo[3.2.1]octan-8-yl)methyl)-1-methyl-1,2,3,4- | | |
| | tetrahydroisoquinoline-2-carbonyl)-4,4-dimethylpent-2- | | |
| | enenitrile | | |
| A72 | 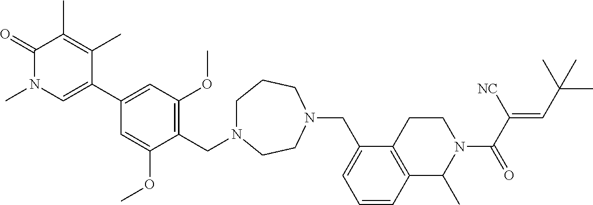 [SMILES: COc1cc(-c2cn(C)c(=O)c(C)c2C)cc(OC)c1CN1CCCN(Cc2cccc3c2CCN(C(=O)/C(C#N)=C/C(C)(C)C)C3C)CC1] | 4a | 681.3/ 7% |
| | (E/Z)-2-(5-((4-(2,6-dimethoxy-4-(1,4,5-trimethyl-6-oxo-1,6- | | |
| | dihydropyridin-3-yl) benzyl)-1,4-diazepan-1-yl) methyl)-1- | | |
| | methyl-1,2,3,4-tetrahydroisoquinoline-2-carbonyl)-4,4- | | |
| | dimethylpent-2-enenitrile | | |
| A70 | 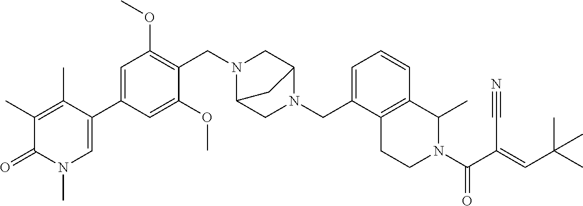 [SMILES: COc1cc(-c2cn(C)c(=O)c(C)c2C)cc(OC)c1CN1CC2CC1CN2Cc1cccc2c1CCN(C(=O)/C(C#N)=C/C(C)(C)C)C2C] | 4a | 678.5/ 14% |
| | (E/Z)-2-(5-((5-(2,6-dimethoxy-4-(1,4,5-trimethyl-6-oxo-1,6- | | |
| | dihydropyridin-3-yl) benzyl)-2,5-diazabicyclo[2.2.1]heptan- | | |
| | 2-yl) methyl)-1-methyl-1,2,3,4-tetrahydroisoquinoline-2- | | |
| | carbonyl)-4,4-dimethylpent-2-enenitrile | | |
| A71 | 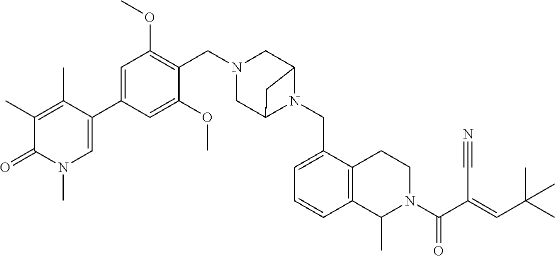 [SMILES: COc1cc(-c2cn(C)c(=O)c(C)c2C)cc(OC)c1CN1CC2CC(C1)N2Cc1cccc2c1CCN(C(=O)/C(C#N)=C/C(C)(C)C)C2C] | 4a | 678.4/ 19% |
| | (E/Z)-2-(5-((3-(2,6-dimethoxy-4-(1,4,5-trimethyl-6-oxo-1,6- | | |
| | dihydropyridin-3-yl)benzyl)-3,6-diazabicyclo[3.1.1]heptan- | | |
| | 6-yl)methyl)-1-methyl-1,2,3,4-tetrahydroisoquinoline-2- | | |
| | carbonyl)-4,4-dimethylpent-2-enenitrile | | |
| B3 | 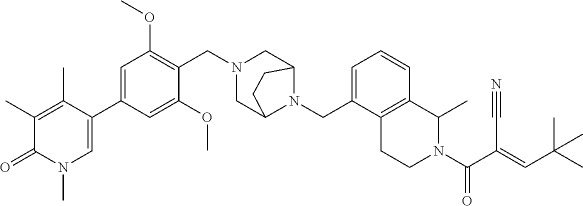 [SMILES: COc1cc(-c2cn(C)c(=O)c(C)c2C)cc(OC)c1CN1CC2CCC(C1)N2Cc1cccc2c1CCN(C(=O)/C(C#N)=C/C(C)(C)C)C2C] | 4a | 692.4/ 5% |
| | (E/Z)-2-(5-((3-(2,6-dimethoxy-4-(1,4,5-trimethyl-6-oxo-1,6- | | |
| | dihydropyridin-3-yl)benzyl)-3,8-diazabicyclo[3.2.1]octan-8- | | |
| | yl)methyl)-1-methyl-1,2,3,4-tetrahydroisoquinoline-2- | | |
| | carbonyl)-4,4-dimethylpent-2-enenitrile | | |
| B13 | 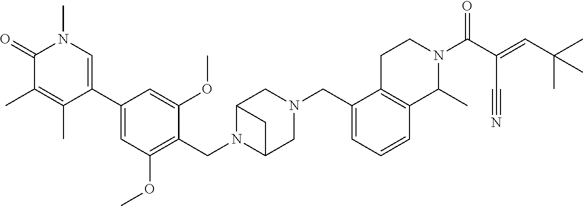 [SMILES: COc1cc(-c2cn(C)c(=O)c(C)c2C)cc(OC)c1CN1C2CC1CN(Cc1cccc3c1CCN(C(=O)/C(C#N)=C/C(C)(C)C)C3C)C2] | 4a | 678.4/ 19% |
| | (E/Z)-2-(5-((6-(2,6-dimethoxy-4-(1,4,5-trimethyl-6-oxo-1,6- | | |
| | dihydropyridin-3-yl)benzyl)-3,6-diazabicyclo[3.1.1]heptan- | | |
| | 3-yl)methyl)-1-methyl-1,2,3,4-tetrahydroisoquinoline-2- | | |
| | carbonyl)-4,4-dimethylpent-2-enenitrile | | |
| B158 | 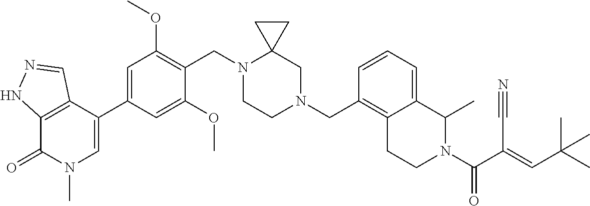 [SMILES: COc1cc(-c2cn(C)c(=O)c3[nH]ncc23)cc(OC)c1CN1CCN(Cc2cccc3c2CCN(C(=O)/C(C#N)=C/C(C)(C)C)C3C)CC12CC2] | 4 | 704.4/ 3% |
| | (E/Z)-2-(5-((4-(2,6-dimethoxy-4-(6-methyl-7-oxo-6,7- | | |
| | dihydro-1H-pyrazolo[3,4-c]pyridin-4-yl)benzyl)-4,7- | | |
| | diazaspiro[2.5]octan-7-yl)methyl)-1-methyl-1,2,3,4- | | |
| | tetrahydroisoquinoline-2-carbonyl)-4,4-dimethylpent-2- | | |
| | enenitrile | | |
| A50 | 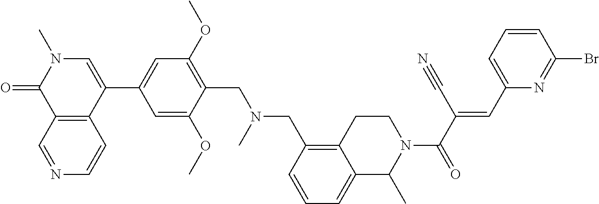 [SMILES: COc1cc(-c2cn(C)c(=O)c3cnccc23)cc(OC)c1CN(C)Cc1cccc2c1CCN(C(=O)/C(C#N)=C/c1cccc(Br)n1)C2C] | 4 | 733.0/ 9% |
| | (E/Z)-3-(6-bromo-2-pyridyl)-2-[5-[[2,6-dimethoxy-4-(2- | | |
| | methyl-1-oxo-2,7-naphthyridin-4-yl)phenyl]methyl-methyl- | | |
| | amino]methyl]-1-methyl-3,4-dihydro-1H-isoquinoline-2- | | |
| | carbonyl]prop-2-enenitrile | | |
| A66 | 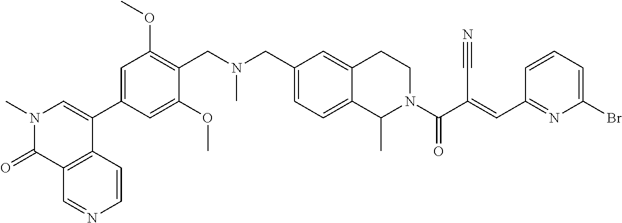 [SMILES: COc1cc(-c2cn(C)c(=O)c3cnccc23)cc(OC)c1CN(C)Cc1ccc2c(c1)CCN(C(=O)/C(C#N)=C/c1cccc(Br)n1)C2C] | 4 | 733.0/ 22% |
| | (E/Z)-3-(3-bromopyridin-2-yl)-2-(6-(((2,6-dimethoxy-4-(2- | | |
| | methyl-1-oxo-1,2-dihydro-2,7-naphthyridin-4- | | |
| | yl)benzyl)(methyl)amino)methyl)-1-methyl-1,2,3,4- | | |
| | tetrahydroisoquinoline-2-carbonyl)acrylonitrile | | |
| A59 | 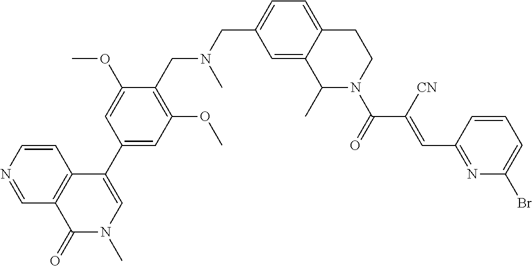 [SMILES: COc1cc(-c2cn(C)c(=O)c3cnccc23)cc(OC)c1CN(C)Cc1ccc2c(c1)C(C)N(C(=O)/C(C#N)=C/c1cccc(Br)n1)CC2] | 4 | 733.0/ 6% |
| | (E/Z)-3-(6-bromo-3-pyridyl)-2-[7-[2,6-dimethoxy-4-(2- | | |
| | methyl-1-oxo-2,7-naphthyridin-4-yl)phenyl]methyl-methyl- | | |
| | amino]methyl]-1-methyl-3,4-dihydro-1H-isoquinoline-2- | | |
| | carbonyl]prop-2-enenitrile | | |
| A52 | 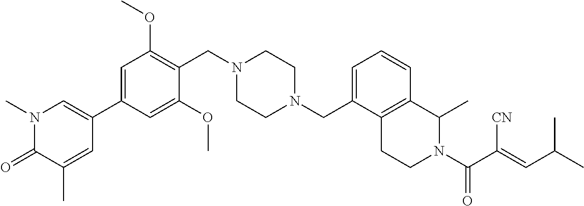 [SMILES: COc1cc(-c2cc(C)c(=O)n(C)c2)cc(OC)c1CN1CCN(Cc2cccc3c2CCN(C(=O)/C(C#N)=C/C(C)C)C3C)CC1] | 4 | 638.3/ 15% |
| | (E/Z)-2-(5-((4-(4-(1,5-dimethyl-6-oxo-1,6-dihydropyridin-3- | | |
| | yl)-2,6-dimethoxybenzyl) piperazin-1-yl)methyl)-1-methyl- | | |
| | 1,2,3,4-tetrahydroisoquinoline-2-carbonyl)-4-methylpent-2- | | |
| | enenitrile | | |
| A60 | 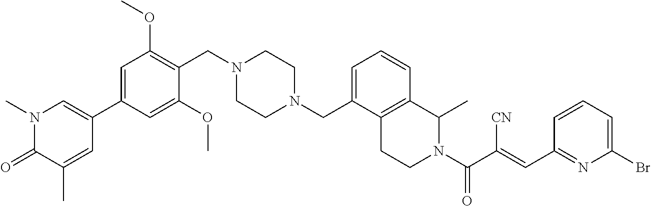 [SMILES: COc1cc(-c2cc(C)c(=O)n(C)c2)cc(OC)c1CN1CCN(Cc2cccc3c2CCN(C(=O)/C(C#N)=C/c2cccc(Br)n2)C3C)CC1] | 4 | 751.2/ 34% |
| | (E/Z)-3-(6-bromopyridin-2-yl)-2-(5-((4-(4-(1,5-dimethyl-6- | | |
| | oxo-1,6-dihydropyridin-3-yl)-2,6-dimethoxy) benzyl) | | |
| | piperazin-1-yl) methyl)-1-methyl-1,2,3,4- | | |
| | tetrahydroisoquinoline-2-carbonyl)acrylonitrile | | |
| A61 | 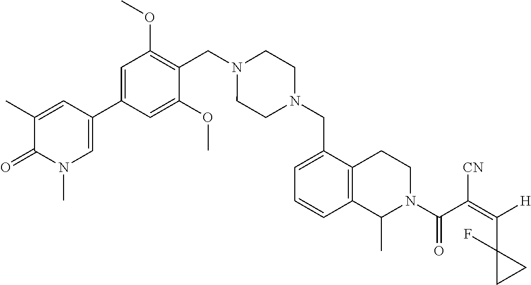 [SMILES: COc1cc(-c2cc(C)c(=O)n(C)c2)cc(OC)c1CN1CCN(Cc2cccc3c2CCN(C(=O)/C(C#N)=C\C2(F)CC2)C3C)CC1] | 4 | 654.3/ 24% |
| | (E/Z)-2-(5-((4-(4-(1,5-dimethyl-6-oxo-1,6-dihydropyridin-3- | | |
| | yl)-2,6-dimethoxybenzyl) piperazin-1-yl) methyl)-1-methyl- | | |
| | 1,2,3,4-tetrahydroisoquinoline-2-carbonyl)-3-(1- | | |
| | fluorocyclopropyl) acrylonitrile | | |
| A69 | 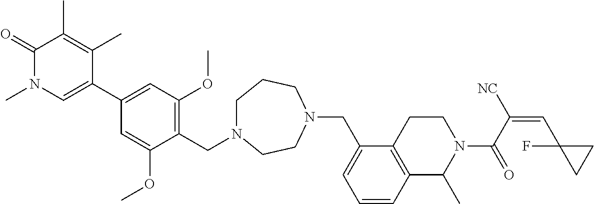 [SMILES: COc1cc(-c2cn(C)c(=O)c(C)c2C)cc(OC)c1CN1CCCN(Cc2cccc3c2CCN(C(=O)/C(C#N)=C\C2(F)CC2)C3C)CC1] | 4 | 682.4/ 4% |
| | (E/Z)-2-(5-((4-(2,6-dimethoxy-4-(1,4,5-trimethyl-6-oxo-1,6- | | |
| | dihydropyridin-3-yl) benzyl)-1,4-diazepan-1-yl) methyl)-1- | | |
| | methyl-1,2,3,4-tetrahydroisoquinoline-2-carbonyl)-3-(1- | | |
| | fluorocyclopropyl)acrylonitrile | | |
| A64 | 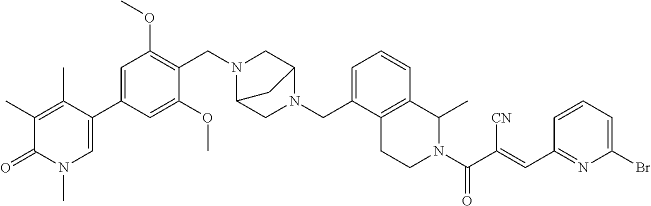 [SMILES: COc1cc(-c2cn(C)c(=O)c(C)c2C)cc(OC)c1CN1CC2CC1CN2Cc1cccc2c1CCN(C(=O)/C(C#N)=C/c1cccc(Br)n1)C2C] | 4a | 780.2/ 7% |
| | (E/Z)-3-(6-bromopyridin-2-yl)-2-(5-((5-(2,6-dimethoxy-4- | | |
| | (1,4,5-trimethyl-6-oxo-1,6-dihydropyridin-3-yl)benzyl)-2,5- | | |
| | diazabicyclo[2.2.1]heptan-2-yl)methyl)-1-methyl-1,2,3,4- | | |
| | tetrahydroisoquinoline-2-carbonyl)acrylonitrile | | |
| B66 | 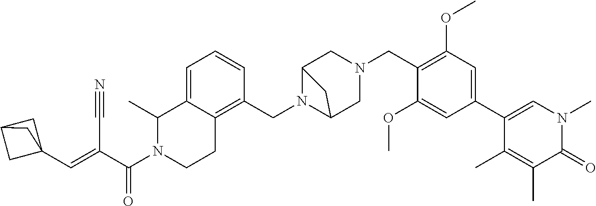 [SMILES: COc1cc(-c2cn(C)c(=O)c(C)c2C)cc(OC)c1CN1CC2CC(C1)N2Cc1cccc2c1CCN(C(=O)/C(C#N)=C/C13CC(C1)C3)C2C] | 17c (using aldehyde prepared according to US2018/ 99931) | 708.4/ 4% |
| | (E/Z)-3-(bicyclo[1.1.1]pentan-1-yl)-2-(5-((3-(2,6- | | |
| | dimethoxy-4-(1,4,5-trimethyl-6-oxo-1,6-dihydropyridin-3- | | |
| | yl)benzyl)-3,6-diazabicyclo[3.1.1]heptan-6-yl)methyl)-1- | | |
| | methyl-1,2,3,4-tetrahydroisoquinoline-2- | | |
| | carbonyl)acrylonitrile | | |
| B68 | 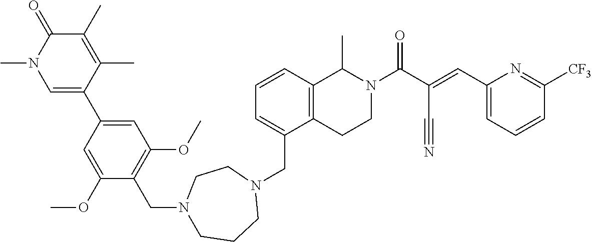 [SMILES: COc1cc(-c2cn(C)c(=O)c(C)c2C)cc(OC)c1CN1CCCN(Cc2cccc3c2CCN(C(=O)/C(C#N)=C/c2cccc(C(F)(F)F)n2)C3C)CC1] | 4 | 769.3/ 22% |
| | (E/Z)-2-(5-((4-(2,6-dimethoxy-4-(1,4,5-trimethyl-6-oxo-1,6- | | |
| | dihydropyridin-3-yl)benzyl)-1,4-diazepan-1-yl)methyl)-1- | | |
| | methyl-1,2,3,4-tetrahydroisoquinoline-2-carbonyl)-3-(6- | | |
| | (trifluoromethyl)pyridin-2-yl)acrylonitrile | | |
| B80 | 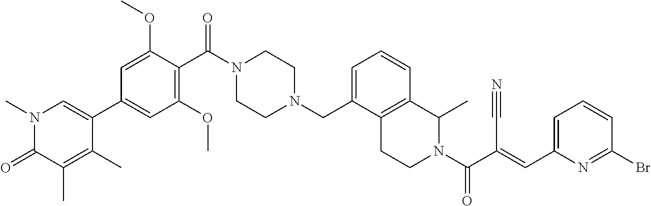 [SMILES: COc1cc(-c2cn(C)c(=O)c(C)c2C)cc(OC)c1C(=O)N1CCN(Cc2cccc3c2CCN(C(=O)/C(C#N)=C/c2cccc(Br)n2)C3C)CC1] | 4 (EtOH:H2O) | 681.3/ 46% |
| | (E/Z)-3-(6-bromopyridin-2-yl)-2-(5-((4-(2,6-dimethoxy-4- | | |
| | (1,4,5-trimethyl-6-oxo-1,6-dihydropyridin-3- | | |
| | yl)benzoyl)piperazin-1-yl)methyl)-1-methyl-1,2,3,4- | | |
| | tetrahydroisoquinoline-2-carbonyl)acrylonitrile | | |
| B118 | 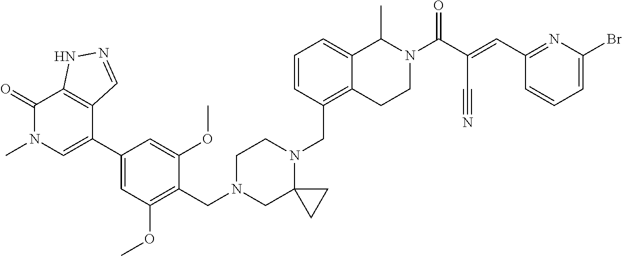 [SMILES: COc1cc(-c2cn(C)c(=O)c3[nH]ncc23)cc(OC)c1CN1CCN(Cc2cccc3c2CCN(C(=O)/C(C#N)=C/c2cccc(Br)n2)C3C)C2(CC2)C1] | 4a | 803.4/ 45% |
| | (E/Z)-3-(6-bromopyridin-2-yl)-2-(5-((7-(2,6-dimethoxy-4-(6- | | |
| | methyl-7-oxo-6,7-dihydro-1H-pyrazolo[3,4-c]pyridin-4- | | |
| | yl)benzyl)-4,7-diazaspiro[2.5]octan-4-yl)methyl)-1-methyl- | | |
| | 1,2,3,4-tetrahydroisoquinoline-2-carbonyl)acrylonitrile | | |
| B108 | 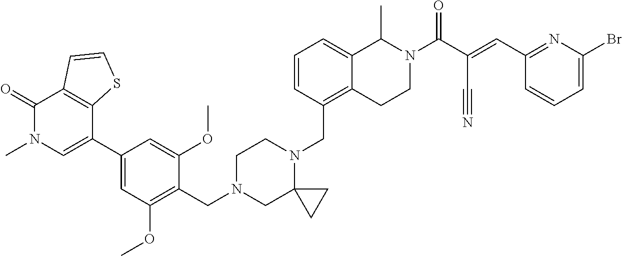 [SMILES: COc1cc(-c2cn(C)c(=O)c3ccsc23)cc(OC)c1CN1CCN(Cc2cccc3c2CCN(C(=O)/C(C#N)=C/c2cccc(Br)n2)C3C)C2(CC2)C1] | 4 | 819.2/ 23% |
| | (E/Z)-3-(6-bromopyridin-2-yl)-2-(5-((7-(2,6-dimethoxy-4-(5- | | |
| | methyl-4-oxo-4,5-dihydrothieno[3,2-c]pyridin-7-yl)benzyl)- | | |
| | 4,7-diazaspiro[2.5]octan-4-yl)methyl)-1-methyl-1,2,3,4- | | |
| | tetrahydroisoquinoline-2-carbonyl)acrylonitrile | | |
| B21 | 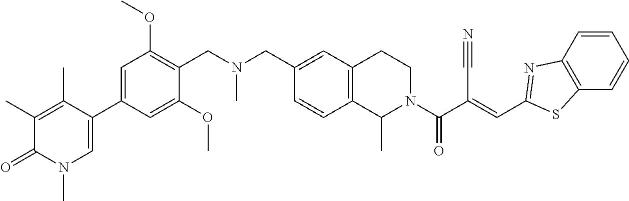 [SMILES: COc1cc(-c2cn(C)c(=O)c(C)c2C)cc(OC)c1CN(C)Cc1ccc2c(c1)CCN(C(=O)/C(C#N)=C/c1nc3ccccc3s1)C2C] | 4 | 688.3/ 2% |
| | (E/Z)-3-(benzo[d]thiazol-2-yl)-2-(6-(((2,6-dimethoxy-4- | | |
| | (1,4,5-trimethyl-6-oxo-1,6-dihydropyridin-3- | | |
| | yl)benzyl)(methyl)amino)methyl)-1-methyl-1,2,3,4- | | |
| | tetrahydroisoquinoline-2-carbonyl)acrylonitrile | | |
| B8 | 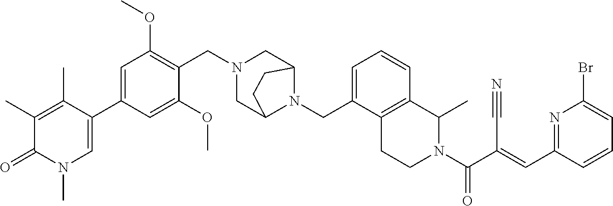 [SMILES: COc1cc(-c2cn(C)c(=O)c(C)c2C)cc(OC)c1CN1CC2CCC(C1)N2Cc1cccc2c1CCN(C(=O)/C(C#N)=C/c1cccc(Br)n1)C2C] | 4 | 791.2/ 19% |
| | (E/Z)-3-(6-bromopyridin-2-yl)-2-(5-((3-(2,6-dimethoxy-4- | | |
| | (1,4,5-trimethyl-6-oxo-1,6-dihydropyridin-3-yl)benzyl)-3,8- | | |
| | diazabicyclo[3.2.1]octan-8-yl)methyl)-1-methyl-1,2,3,4- | | |
| | tetrahydroisoquinoline-2-carbonyl)acrylonitrile | | |
| B25 | 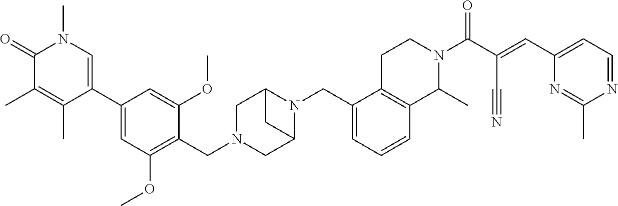 [SMILES: COc1cc(-c2cn(C)c(=O)c(C)c2C)cc(OC)c1CN1CC2CC(C1)N2Cc1cccc2c1CCN(C(=O)/C(C#N)=C/c1ccnc(C)n1)C2C] | 4 (in DMF) | 714.3/ 29% |
| | (E/Z)-2-(5-((3-(2,6-dimethoxy-4-(1,4,5-trimethyl-6-oxo-1,6- | | |
| | dihydropyridin-3-yl)benzyl)-3,6-diazabicyclo[3.1.1]heptan- | | |
| | 6-yl)methyl)-1-methyl-1,2,3,4-tetrahydroisoquinoline-2- | | |
| | carbonyl)-3-(2-methylpyrimidin-4-yl)acrylonitrile | | |
| B7 | 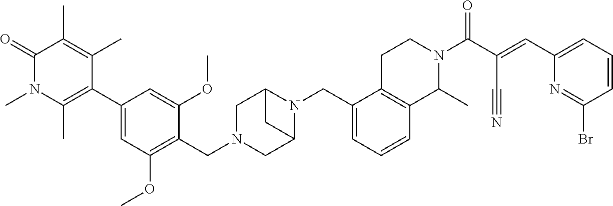 [SMILES: COc1cc(-c2c(C)c(C)c(=O)n(C)c2C)cc(OC)c1CN1CC2CC(C1)N2Cc1cccc2c1CCN(C(=O)/C(C#N)=C/c1cccc(Br)n1)C2C] | 4a | 777.2/ 12% |
| | (E/Z)-3-(6-bromopyridin-2-yl)-2-(5-((3-(2,6-dimethoxy-4- | | |
| | (1,4,5-trimethyl-6-oxo-1,6-dihydropyridin-3-yl)benzyl)-3,6- | | |
| | diazabicyclo[3.1.1]heptan-6-yl)methyl)-1-methyl-1,2,3,4- | | |
| | tetrahydroisoquinoline-2-carbonyl)acrylonitrile | | |
| B96 | 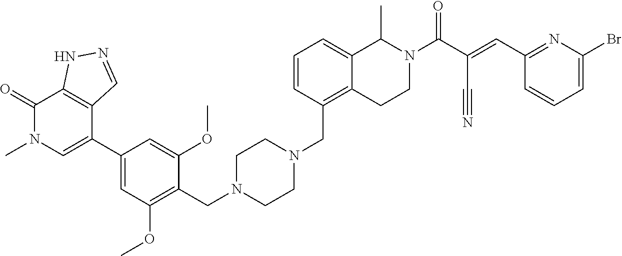 [SMILES: COc1cc(-c2cn(C)c(=O)c3[nH]ncc23)cc(OC)c1CN1CCN(Cc2cccc3c2CCN(C(=O)/C(C#N)=C/c2cccc(Br)n2)C3C)CC1] | 17b | 777.2/ 24% |
| | (E/Z)-3-(6-bromopyridin-2-yl)-2-(5-((4-(2,6-dimethoxy-4-(6- | | |
| | methyl-7-oxo-6,7-dihydro-1H-pyrazolo[3,4-c]pyridin-4- | | |
| | yl)benzyl)piperazin-1-yl)methyl)-1-methyl-1,2,3,4- | | |
| | tetrahydroisoquinoline-2-carbonyl)acrylonitrile | | |
| B109 | 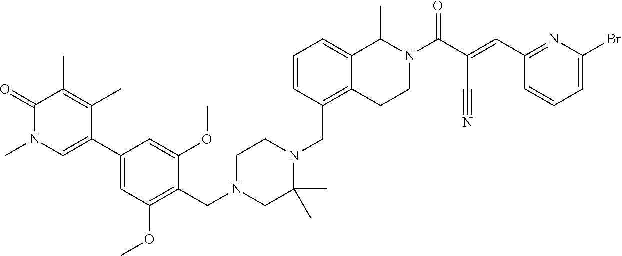 [SMILES: COc1cc(-c2cn(C)c(=O)c(C)c2C)cc(OC)c1CN1CCN(Cc2cccc3c2CCN(C(=O)/C(C#N)=C/c2cccc(Br)n2)C3C)C(C)(C)C1] | 17b | 793.2/ 51% |
| | (E/Z)-3-(6-bromopyridin-2-yl)-2-(5-((4-(2,6-dimethoxy-4- | | |
| | (1,4,5-trimethyl-6-oxo-1,6-dihydropyridin-3-yl)benzyl)-2,2- | | |
| | dimethylpiperazin-1-yl)methyl)-1-methyl-1,2,3,4- | | |
| | tetrahydroisoquinoline-2-carbonyl)acrylonitrile | | |
| B110 | 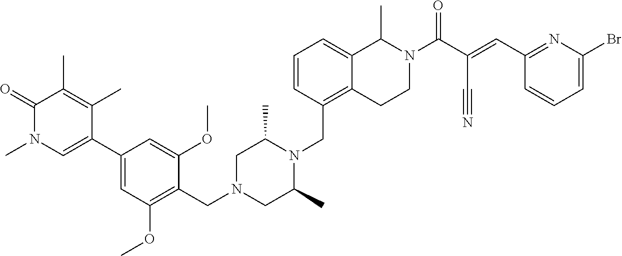 [SMILES: COc1cc(-c2cn(C)c(=O)c(C)c2C)cc(OC)c1CN1C[C@H](C)N(Cc2cccc3c2CCN(C(=O)/C(C#N)=C/c2cccc(Br)n2)C3C)[C@@H](C)C1] | 17b (in ethanol: water) | 794.2/ 51% |
| | (E/Z)-3-(6-bromopyridin-2-yl)-2-(5-(((2S,6S)-4-(2,6- | | |
| | dimethoxy-4-(1,4,5-trimethyl-6-oxo-1,6-dihydropyridin-3- | | |
| | yl)benzyl)-2,6-dimethylpiperazin-1-yl)methyl)-1-methyl- | | |
| | 1,2,3,4-tetrahydroisoquinoline-2-carbonyl)acrylonitrile | | |
| B5 | 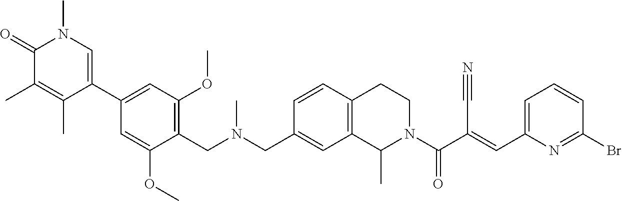 [SMILES: COc1cc(-c2cn(C)c(=O)c(C)c2C)cc(OC)c1CN(C)Cc1ccc2c(c1)C(C)N(C(=O)/C(C#N)=C/c1cccc(Br)n1)CC2] | 4a (in ethanol) | 712.2/ 32% |
| | (E/Z)-3-(6-bromopyridin-2-yl)-2-(7-(((2,6-dimethoxy-4- | | |
| | (1,4,5-trimethyl-6-oxo-1,6-dihydropyridin-3- | | |
| | yl)benzyl)(methyl)amino)methyl)-1-methyl-1,2,3,4- | | |
| | tetrahydroisoquinoline-2-carbonyl)acrylonitrile | | |
| B14 | 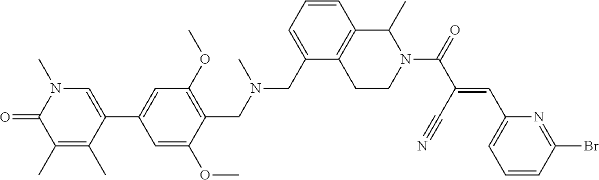 [SMILES: COc1cc(-c2cn(C)c(=O)c(C)c2C)cc(OC)c1CN(C)Cc1cccc2c1CCN(C(=O)/C(C#N)=C/c1cccc(Br)n1)C2C] | 17a (in DMF) | 712.2/ 22% |
| | (E/Z)-3-(6-bromopyridin-2-yl)-2-(5-(((2,6-dimethoxy-4- | | |
| | (1,4,5-trimethyl-6-oxo-1,6-dihydropyridin-3- | | |
| | yl)benzyl)(methyl)amino)methyl)-1-methyl-1,2,3,4- | | |
| | tetrahydroisoquinoline-2-carbonyl)acrylonitrile | | |
| B15 | 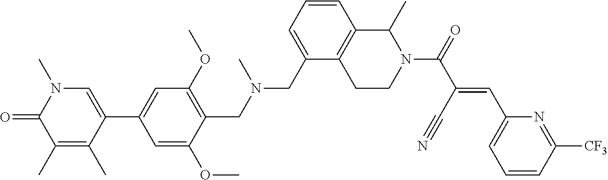 [SMILES: COc1cc(-c2cn(C)c(=O)c(C)c2C)cc(OC)c1CN(C)Cc1cccc2c1CCN(C(=O)/C(C#N)=C/c1cccc(C(F)(F)F)n1)C2C] | 4a | 700.2/ 19% |
| | (E/Z)-2-(5-(((2,6-dimethoxy-4-(1,4,5-trimethyl-6-oxo-1,6- | | |
| | dihydropyridin-3-yl)benzyl)(methyl)amino)methyl)-1- | | |
| | methyl-1,2,3,4-tetrahydroisoquinoline-2-carbonyl)-3-(6- | | |
| | (trifluoromethyl)pyridin-2-yl)acrylonitrile | | |
| B2 | 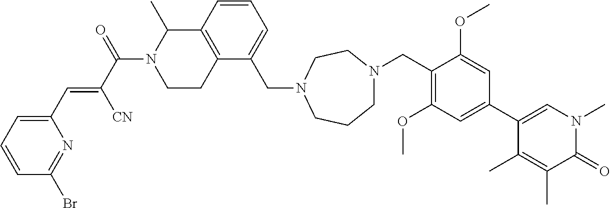 [SMILES: COc1cc(-c2cn(C)c(=O)c(C)c2C)cc(OC)c1CN1CCCN(Cc2cccc3c2CCN(C(=O)/C(C#N)=C/c2cccc(Br)n2)C3C)CC1] | 4a | 779.2/ 6% |
| | (E/Z)-3-(6-bromopyridin-2-yl)-2-(5-((4-(2,6-dimethoxy-4- | | |
| | (1,4,5-trimethyl-6-oxo-1,6-dihydropyridin-3-yl)benzyl)-1,4- | | |
| | diazepan-1-yl)methyl)-1-methyl-1,2,3,4- | | |
| | tetrahydroisoquinoline-2-carbonyl)acrylonitrile | | |
| B132 | 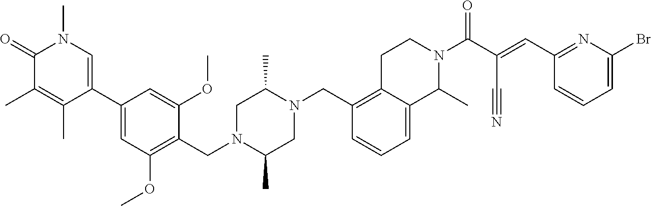 [SMILES: COc1cc(-c2cn(C)c(=O)c(C)c2C)cc(OC)c1CN1C[C@H](C)N(Cc2cccc3c2CCN(C(=O)/C(C#N)=C/c2cccc(Br)n2)C3C)C[C@H]1C] | 4a | 794.2/ 74% |
| | (E/Z)-3-(6-bromopyridin-2-yl)-2-(5-(((2R,5S)-4-(2,6- | | |
| | dimethoxy-4-(1,4,5-trimethyl-6-oxo-1,6-dihydropyridin-3- | | |
| | yl)benzyl)-2,5-dimethylpiperazin-1-yl)methyl)-1-methyl- | | |
| | 1,2,3,4-tetrahydroisoquinoline-2-carbonyl)acrylonitrile | | |
| B131 | 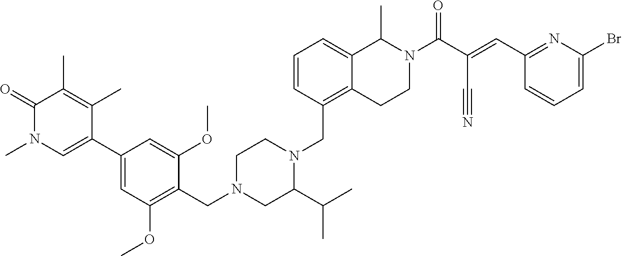 [SMILES: COc1cc(-c2cn(C)c(=O)c(C)c2C)cc(OC)c1CN1CCN(Cc2cccc3c2CCN(C(=O)/C(C#N)=C/c2cccc(Br)n2)C3C)C(C(C)C)C1] | 4a | 808.3/ 12% |
| | (E/Z)-3-(6-bromopyridin-2-yl)-2-(5-((4-(2,6-dimethoxy-4- | | |
| | (1,4,5-trimethyl-6-oxo-1,6-dihydropyridin-3-yl)benzyl)-2- | | |
| | isopropylpiperazin-1-yl)methyl)-1-methyl-1,2,3,4- | | |
| | tetrahydroisoquinoline-2-carbonyl)acrylonitrile | | |
| B61 | 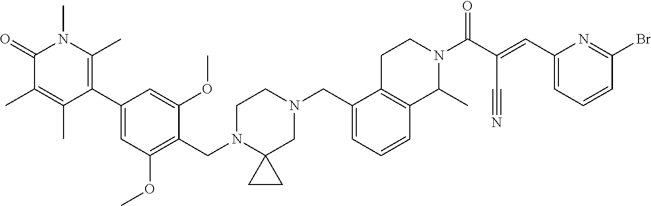 [SMILES: COc1cc(-c2c(C)c(C)c(=O)n(C)c2C)cc(OC)c1CN1CCN(Cc2cccc3c2CCN(C(=O)/C(C#N)=C/c2cccc(Br)n2)C3C)CC12CC2] | 4a | 793.1/ 42% |
| | (E/Z)-3-(6-bromopyridin-2-yl)-2-(5-((4-(2,6-dimethoxy-4- | | |
| | (1,4,5-trimethyl-6-oxo-1,6-dihydropyridin-3-yl)benzyl)-4,7- | | |
| | diazaspiro[2.5]octan-7-yl)methyl)-1-methyl-1,2,3,4- | | |
| | tetrahydroisoquinoline-2-carbonyl)acrylonitrile | | |
| BRD9g | 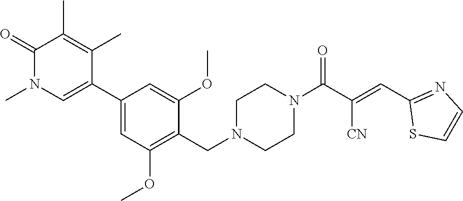 [SMILES: COc1cc(-c2cn(C)c(=O)c(C)c2C)cc(OC)c1CN1CCN(C(=O)/C(C#N)=C/c2nccs2)CC1] | 4 | 592.2/ 18% |
| | (E/Z)-2-(4-(2,6-dimethoxy-4-(1,4,5-trimethyl-6-oxo-1,6- | | |
| | dihydropyridin-3-yl)benzyl)piperazine-1-carbonyl)-3- | | |
| | (thiazol-2-yl)acrylonitrile | | |
| BRD9h | 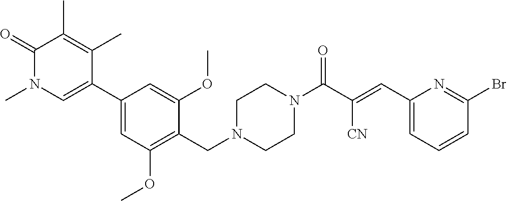 [SMILES: COc1cc(-c2cn(C)c(=O)c(C)c2C)cc(OC)c1CN1CCN(C(=O)/C(C#N)=C/c2cccc(Br)n2)CC1] | 4 | 608.2/ 14% |
| | (E/Z)-3-(6-bromopyridin-2-yl)-2-(4-(2,6-dimethoxy-4- | | |
| | (1,4,5-trimethyl-6-oxo-1,6-dihydropyridin-3- | | |
| | yl)benzyl)piperazine-1-carbonyl)acrylonitrile | | |
| BRD9i | 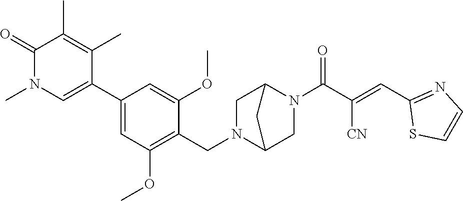 [SMILES: COc1cc(-c2cn(C)c(=O)c(C)c2C)cc(OC)c1CN1CC2CC1CN2C(=O)/C(C#N)=C/c1nccs1] | 4 | 546.2/ 13% |
| | (E/Z)-2-(5-(2,6-dimethoxy-4-(1,4,5-trimethyl-6-oxo-1,6- | | |
| | dihydropyridin-3-yl)benzyl)-2,5- | | |
| | diazabicyclo[2.2.1]heptane-2-carbonyl)-3-(thiazol-2- | | |
| | yl)acrylonitrile | | |
| BRD9]j | 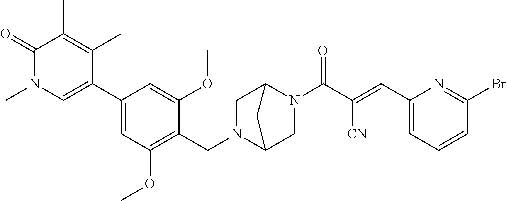 [SMILES: COc1cc(-c2cn(C)c(=O)c(C)c2C)cc(OC)c1CN1CC2CC1CN2C(=O)/C(C#N)=C/c1cccc(Br)n1] | 4 | 619.4/ 12% |
| | (E/Z)-3-(6-bromopyridin-2-yl)-2-(5-(2,6-dimethoxy-4- | | |
| | (1,4,5-trimethyl-6-oxo-1,6-dihydropyridin-3-yl)benzyl)-2,5- | | |
| | diazabicyclo[2.2.1]heptane-2-carbonyl)acrylonitrile | | |
| BRD9k | 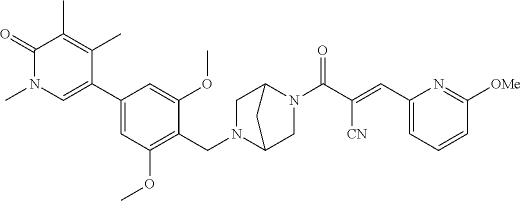 [SMILES: COc1cccc(/C=C(\C#N)C(=O)N2CC3CC2CN3Cc2c(OC)cc(-c3cn(C)c(=O)c(C)c3C)cc2OC)n1] | 4 | 570.5/ 16% |
| | (E/Z)-2-(5-(2,6-dimethoxy-4-(1,4,5-trimethyl-6-oxo-1,6- | | |
| | dihydropyridin-3-yl)benzyl)-2,5- | | |
| | diazabicyclo[2.2.1]heptane-2-carbonyl)-3-(6- | | |
| | methoxypyridin-2-yl)acrylonitrile | | |
| BRD9x | 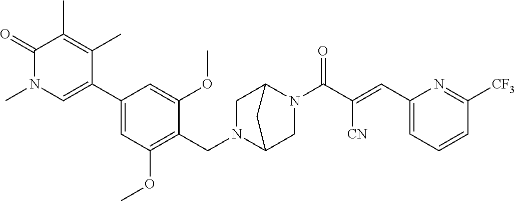 [SMILES: COc1cc(-c2cn(C)c(=O)c(C)c2C)cc(OC)c1CN1CC2CC1CN2C(=O)/C(C#N)=C/c1cccc(C(F)(F)F)n1] | 4 | 608.5/ 12% |
| | (E/Z)-2-(5-(2,6-dimethoxy-4-(1,4,5-trimethyl-6-oxo-1,6- | | |
| | dihydropyridin-3-yl)benzyl)-2,5- | | |
| | diazabicyclo[2.2.1]heptane-2-carbonyl)-3-(6- | | |
| | (trifluoromethyl)pyridin-2-yl)acrylonitrile | | |
| BRD9l | 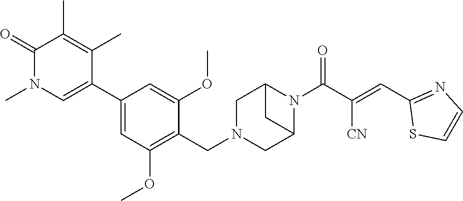 [SMILES: COc1cc(-c2cn(C)c(=O)c(C)c2C)cc(OC)c1CN1CC2CC(C1)N2C(=O)/C(C#N)=C/c1nccs1] | 4 | 546.2/1 8% |
| | (E/Z)-2-(3-(2,6-dimethoxy-4-(1,4,5-trimethyl-6-oxo-1,6- | | |
| | dihydropyridin-3-yl)benzyl)-3,6- | | |
| | diazabicyclo[3.1.1]heptane-6-carbonyl)-3-(thiazol-2- | | |
| | yl)acrylonitrile | | |
| BRD9m | 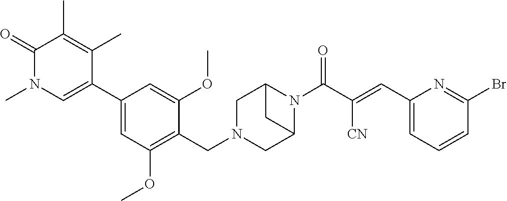 [SMILES: COc1cc(-c2cn(C)c(=O)c(C)c2C)cc(OC)c1CN1CC2CC(C1)N2C(=O)/C(C#N)=C/c1cccc(Br)n1] | 4 | 619.2/ 2% |
| | (E/Z)-3-(6-bromopyridin-2-yl)-2-(3-(2,6-dimethoxy-4- | | |
| | (1,4,5-trimethyl-6-oxo-1,6-dihydropyridin-3-yl)benzyl)-3,6- | | |
| | diazabicyclo[3.1.1]heptane-6-carbonyl)acrylonitrile | | |
| BRD9n | 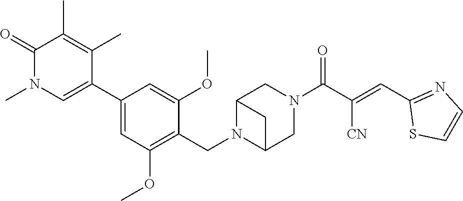 [SMILES: COc1cc(-c2cn(C)c(=O)c(C)c2C)cc(OC)c1CN1C2CC1CN(C(=O)/C(C#N)=C/c1nccs1)C2] | 4 | 546.2/ 13% |
| | (E/Z)-2-(6-(2,6-dimethoxy-4-(1,4,5-trimethyl-6-oxo-1,6- | | |
| | dihydropyridin-3-yl)benzyl)-3,6- | | |
| | diazabicyclo[3.1.1]heptane-3-carbonyl)-3-(thiazol-2- | | |
| | yl)acrylonitrile | | |
| BRD9o | 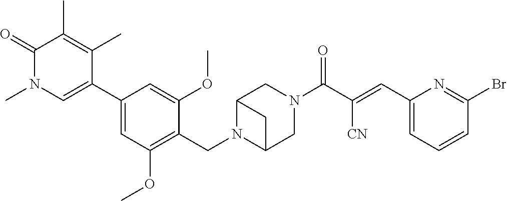 [SMILES: COc1cc(-c2cn(C)c(=O)c(C)c2C)cc(OC)c1CN1C2CC1CN(C(=O)/C(C#N)=C/c1cccc(Br)n1)C2] | 4 | 619.2/ 6% |
| | (E/Z)-3-(6-bromopyridin-2-yl)-2-(6-(2,6-dimethoxy-4- | | |
| | (1,4,5-trimethyl-6-oxo-1,6-dihydropyridin-3-yl)benzyl)-3,6- | | |
| | diazabicyclo[3.1.1]heptane-3-carbonyl)acrylonitrile | | |
| BRD9p | 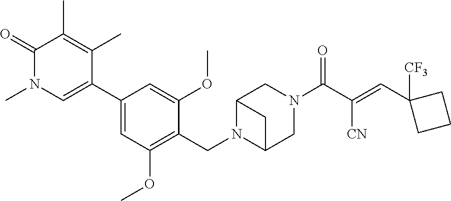 [SMILES: COc1cc(-c2cn(C)c(=O)c(C)c2C)cc(OC)c1CN1C2CC1CN(C(=O)/C(C#N)=C/C1(C(F)(F)F)CCC1)C2] | 4 | 585.6/ 9% |
| | (E/Z)-2-(6-(2,6-dimethoxy-4-(1,4,5-trimethyl-6-oxo-1,6- | | |
| | dihydropyridin-3-yl)benzyl)-3,6- | | |
| | diazabicyclo[3.1.1]heptane-3-carbonyl)-3-(1- | | |
| | (trifluoromethyl)cyclobutyl)acrylonitrile | | |
| BRD9y | 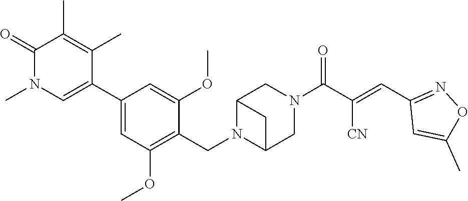 [SMILES: COc1cc(-c2cn(C)c(=O)c(C)c2C)cc(OC)c1CN1C2CC1CN(C(=O)/C(C#N)=C/c1cc(C)on1)C2] | 4 | 544.6/ 23% |
| | (E/Z)-2-(6-(2,6-dimethoxy-4-(1,4,5-trimethyl-6-oxo-1,6- | | |
| | dihydropyridin-3-yl)benzyl)-3,6- | | |
| | diazabicyclo[3.1.1]heptane-3-carbonyl)-3-(5- | | |
| | methylisoxazol-3-yl)acrylonitrile | | |
| B188 | 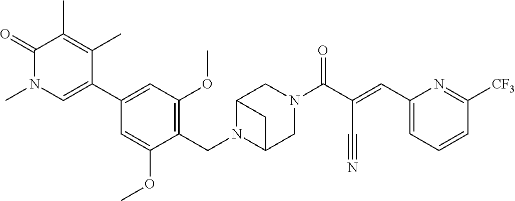 [SMILES: COc1cc(-c2cn(C)c(=O)c(C)c2C)cc(OC)c1CN1C2CC1CN(C(=O)/C(C#N)=C/c1cccc(C(F)(F)F)n1)C2] | 4 | 608.2/ 17% |
| | (E/Z)-2-(6-(2,6-dimethoxy-4-(1,4,5-trimethyl-6-oxo-1,6- | | |
| | dihydropyridin-3-yl)benzyl)-3,6- | | |
| | diazabicyclo[3.1.1]heptane-3-carbonyl)-3-(6- | | |
| | (trifluoromethyl)pyridin-2-yl)acrylonitrile | | |
| B189 | 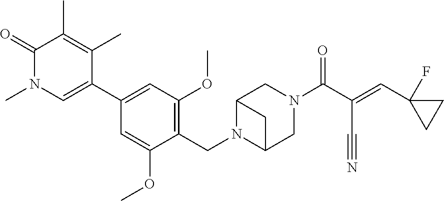 [SMILES: COc1cc(-c2cn(C)c(=O)c(C)c2C)cc(OC)c1CN1C2CC1CN(C(=O)/C(C#N)=C/C1(F)CC1)C2] | 4 | 521.2/ 25% |
| | (E/Z)-2-(6-(2,6-dimethoxy-4-(1,4,5-trimethyl-6-oxo-1,6- | | |
| | dihydropyridin-3-yl)benzyl)-3,6- | | |
| | diazabicyclo[3.1.1]heptane-3-carbonyl)-3-(1- | | |
| | fluorocyclopropyl)acrylonitrile (see WO2020/146194 for | | |
| | preparation of aldehyde) | | |
| BRD9q | 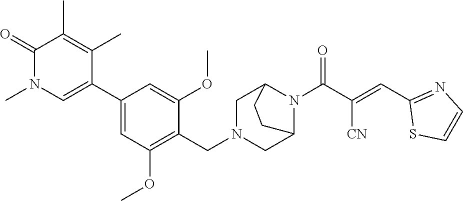 [SMILES: COc1cc(-c2cn(C)c(=O)c(C)c2C)cc(OC)c1CN1CC2CCC(C1)N2C(=O)/C(C#N)=C/c1nccs1] | 4 | 560.2/ 75% |
| | (E/Z)-2-(3-(2,6-dimethoxy-4-(1,4,5-trimethyl-6-oxo-1,6- | | |
| | dihydropyridin-3-yl)benzyl)-3,8-diazabicyclo[3.2.1]octane- | | |
| | 8-carbonyl)-3-(thiazol-2-yl)acrylonitrile | | |
| BRD9z | 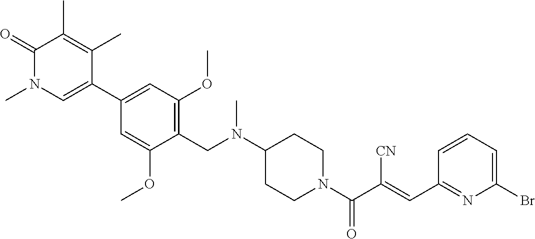 [SMILES: COc1cc(-c2cn(C)c(=O)c(C)c2C)cc(OC)c1CN(C)C1CCN(C(=O)/C(C#N)=C/c2cccc(Br)n2)CC1] | 4 | 634.2/ 4% |
| | (E/Z)-3-(6-bromopyridin-2-yl)-2-(4-((2,6-dimethoxy-4- | | |
| | (1,4,5-trimethyl-6-oxo-1,6-dihydropyridin-3- | | |
| | yl) benzyl)(methyl)amino)piperidine-1-carbonyl)acrylonitrile | | |
| BRD9aa | 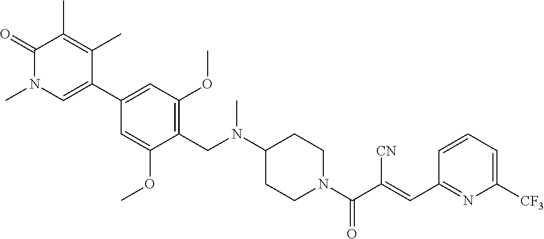 [SMILES: COc1cc(-c2cn(C)c(=O)c(C)c2C)cc(OC)c1CN(C)C1CCN(C(=O)/C(C#N)=C/c2cccc(C(F)(F)F)n2)CC1] | 4 | 624.7/ 10% |
| | (E/Z)-2-(4-((2,6-dimethoxy-4-(1,4,5-trimethyl-6-oxo-1,6- | | |
| | dihydropyridin-3-yl)benzyl)(methyl)amino)piperidine-1- | | |
| | carbonyl)-3-(6-(trifluoromethyl)pyridin-2-yl)acrylonitrile | | |
| B187 | 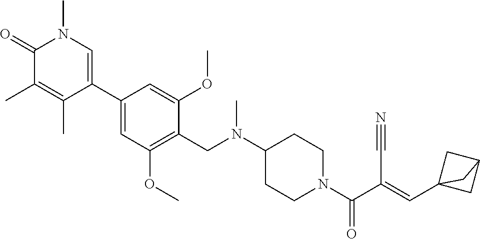 [SMILES: COc1cc(-c2cn(C)c(=O)c(C)c2C)cc(OC)c1CN(C)C1CCN(C(=O)/C(C#N)=C/C23CC(C2)C3)CC1] | 4 | 545.3/ 8% |
| | (E/Z)-3-(bicyclo[1.1.1]pentan-1-yl)-2-(4-((2,6-dimethoxy-4- | | |
| | (1,4,5-trimethyl-6-oxo-1,6-dihydropyridin-3- | | |
| | yl)benzyl)(methyl)amino)piperidine-1-carbonyl)acrylonitrile | | |
| | (see US2018/99931 for preparation of aldehyde) | | |
| BRD9r | 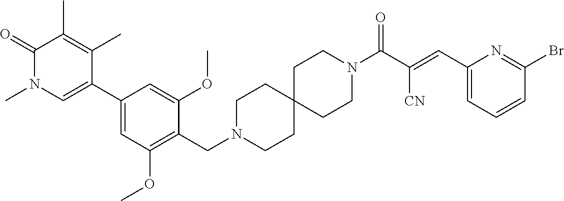 [SMILES: COc1cc(-c2cn(C)c(=O)c(C)c2C)cc(OC)c1CN1CCC2(CC1)CCN(C(=O)/C(C#N)=C/c1cccc(Br)n1)CC2] | 4 | 675.6/ 11% |
| | (E/Z)-3-(6-bromopyridin-2-yl)-2-(9-(2,6-dimethoxy-4- | | |
| | (1,4,5-trimethyl-6-oxo-1,6-dihydropyridin-3-yl)benzyl)-3,9- | | |
| | diazaspiro[5.5]undecane-3-carbonyl)acrylonitrile | | |
| BRD9ab | 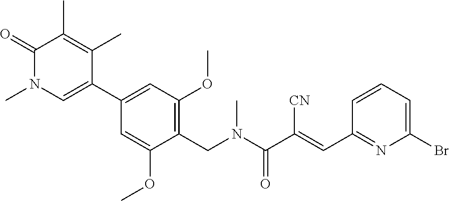 [SMILES: COc1cc(-c2cn(C)c(=O)c(C)c2C)cc(OC)c1CN(C)C(=O)/C(C#N)=C/c1cccc(Br)n1] | 4 | 552.5/ 14% |
| | (E/Z)-3-(6-bromopyridin-2-yl)-2-cyano-N-(2,6-dimethoxy-4- | | |
| | (1,4,5-trimethyl-6-oxo-1,6-dihydropyridin-3-yl)benzyl)-N- | | |
| | methylacrylamide | | |
| B193 | 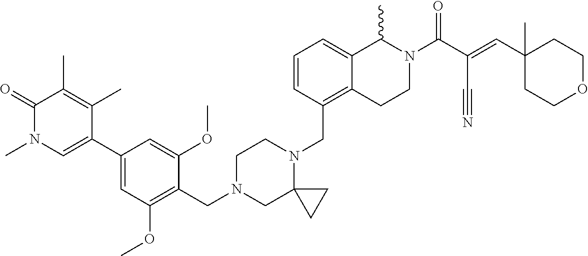 [SMILES: COc1cc(-c2cn(C)c(=O)c(C)c2C)cc(OC)c1CN1CCN(Cc2cccc3c2CCN(C(=O)/C(C#N)=C/C2(C)CCOCC2)C3C)C2(CC2)C1] | 4a | 734.3/ 42% |
| | single enantiomer of unknown absolute configuration | | |
| | (using peak 2 of tert-butyl 5-bromo-1-methyl-3,4- | | |
| | dihydroisoquinoline-2(1H)-carboxylate) | | |
| | (E/Z)-2-(5-((7-(2,6-dimethoxy-4-(1,4,5-trimethyl-6-oxo-1,6- | | |
| | dihydropyridin-3-yl)benzyl)-4,7-diazaspiro[2.5]octan-4- | | |
| | yl)methyl)-1-methyl-1,2,3,4-tetrahydroisoquinoline-2- | | |
| | carbonyl)-3-(4-methyltetrahydro-2H-pyran-4-yl)acrylonitrile | | |
| B194 | 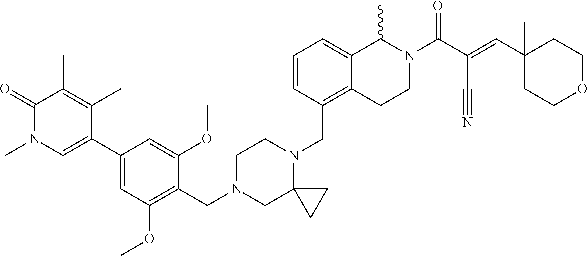 [SMILES: COc1cc(-c2cn(C)c(=O)c(C)c2C)cc(OC)c1CN1CCN(Cc2cccc3c2CCN(C(=O)/C(C#N)=C/C2(C)CCOCC2)C3C)C2(CC2)C1] | 4a | 734.3/ 41% |
| | single enantiomer of unknown absolute configuration | | |
| | (using peak 1 of tert-butyl 5-bromo-1-methyl-3,4- | | |
| | dihydroisoquinoline-2(1H)-carboxylate) | | |
| | (E/Z)-2-(5-((7-(2,6-dimethoxy-4-(1,4,5-trimethyl-6-oxo-1,6- | | |
| | dihydropyridin-3-yl)benzyl)-4,7-diazaspiro[2.5]octan-4- | | |
| | yl)methyl)-1-methyl-1,2,3,4-tetrahydroisoquinoline-2- | | |
| | carbonyl)-3-(4-methyltetrahydro-2H-pyran-4-yl)acrylonitrile | | |
| B195 | 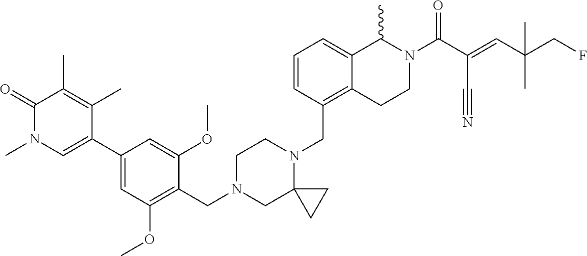 [SMILES: COc1cc(-c2cn(C)c(=O)c(C)c2C)cc(OC)c1CN1CCN(Cc2cccc3c2CCN(C(=O)/C(C#N)=C/C(C)(C)CF)C3C)C2(CC2)C1] | 4a | 710.4/ 26% |
| | single enantiomer of unknown absolute configuration | | |
| | (using peak 1 of tert-butyl 5-bromo-1-methyl-3,4- | | |
| | dihydroisoquinoline-2(1H)-carboxylate) | | |
| | (E/Z)-2-(5-((7-(2,6-dimethoxy-4-(1,4,5-trimethyl-6-oxo-1,6- | | |
| | dihydropyridin-3-yl)benzyl)-4,7-diazaspiro[2.5]octan-4- | | |
| | yl)methyl)-1-methyl-1,2,3,4-tetrahydroisoquinoline-2- | | |
| | carbonyl)-5-fluoro-4,4-dimethylpent-2-enenitrile | | |
| B196 | 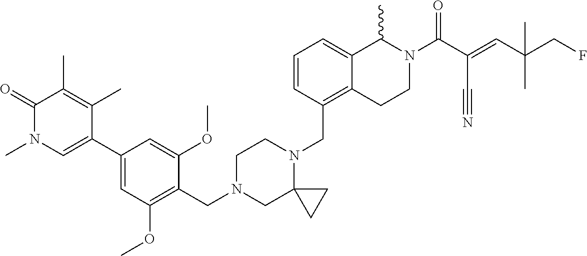 [SMILES: COc1cc(-c2cn(C)c(=O)c(C)c2C)cc(OC)c1CN1CCN(Cc2cccc3c2CCN(C(=O)/C(C#N)=C/C(C)(C)CF)C3C)C2(CC2)C1] | 4a | 710.4/ 26% |
| | single enantiomer of unknown absolute configuration | | |
| | (using peak 2 of tert-butyl 5-bromo-1-methyl-3,4- | | |
| | dihydroisoquinoline-2(1H)-carboxylate) | | |
| | (E/Z)-2-(5-((7-(2,6-dimethoxy-4-(1,4,5-trimethyl-6-oxo-1,6- | | |
| | dihydropyridin-3-yl)benzyl)-4,7-diazaspiro[2.5]octan-4- | | |
| | yl)methyl)-1-methyl-1,2,3,4-tetrahydroisoquinoline-2- | | |
| | carbonyl)-5-fluoro-4,4-dimethylpent-2-enenitrile | | |
| C19 | 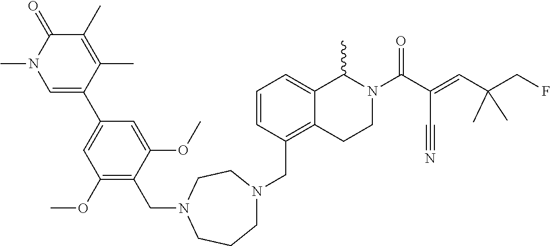 [SMILES: COc1cc(-c2cn(C)c(=O)c(C)c2C)cc(OC)c1CN1CCCN(Cc2cccc3c2CCN(C(=O)/C(C#N)=C/C(C)(C)CF)C3C)CC1] | 4 (in DMF) | 698.2/ 15% |
| | single enantiomer of unknown absolute configuration | | |
| | (using peak 2 of tert-butyl 5-bromo-1-methyl-3,4- | | |
| | dihydroisoquinoline-2(1H)-carboxylate) | | |
| C20 | 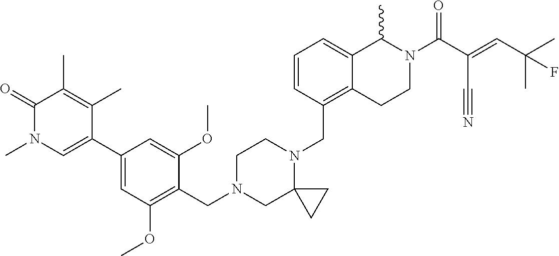 [SMILES: COc1cc(-c2cn(C)c(=O)c(C)c2C)cc(OC)c1CN1CCN(Cc2cccc3c2CCN(C(=O)/C(C#N)=C/C(C)(C)F)C3C)C2(CC2)C1] | 4 (in DMF) | 696.2/ 7% |
| | single enantiomer of unknown absolute configuration | | |
| | (using peak 2 of tert-butyl 5-bromo-1-methyl-3,4- | | |
| | dihydroisoquinoline-2(1H)-carboxylate) | | |
| | (E/Z)-2-(5-((7-(2,6-dimethoxy-4-(1,4,5-trimethyl-6-oxo-1,6- | | |
| | dihydropyridin-3-yl)benzyl)-4,7-diazaspiro[2.5]octan-4- | | |
| | yl)methyl)-1-methyl-1,2,3,4-tetrahydroisoquinoline-2- | | |
| | carbonyl)-4-fluoro-4-methylpent-2-enenitrile | | |
| C21 | 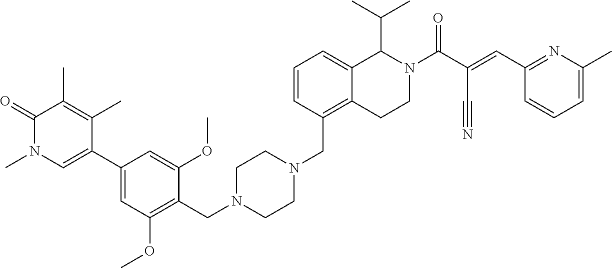 | 4a | 795.2/ 15% |
| | (E/Z)-3-(6-bromopyridin-2-yl)-2-(5-((4-(2,6-dimethoxy-4- | | |
| | (1,4,5-trimethyl-6-oxo-1,6-dihydropyridin-3- | | |
| | yl)benzyl)piperazin-1-yl)methyl)-1-isopropyl-1,2,3,4- | | |
| | tetrahydroisoquinoline-2-carbonyl)acrylonitrile | | |
| C22 | 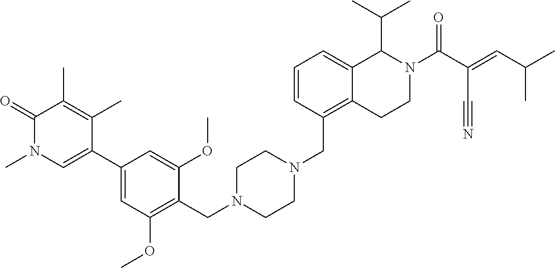 [SMILES: COc1cc(-c2cn(C)c(=O)c(C)c2C)cc(OC)c1CN1CCN(Cc2cccc3c2CCN(C(=O)/C(C#N)=C/C(C)C)C3C(C)C)CC1] | 4 | 680.4/ 2% |
| | (E/Z)-2-(5-((4-(2,6-dimethoxy-4-(1,4,5-trimethyl-6-oxo-1,6- | | |
| | dihydropyridin-3-yl)benzyl)piperazin-1-yl)methyl)-1- | | |
| | isopropyl-1,2,3,4-tetrahydroisoquinoline-2-carbonyl)-4- | | |
| | methylpent-2-enenitrile | | |
| C23 | 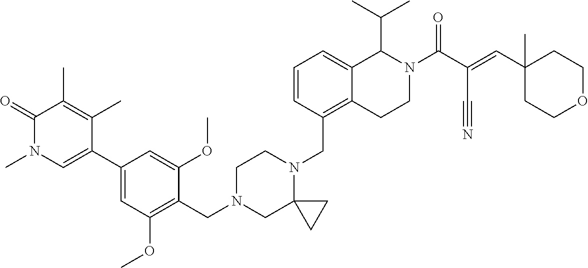 [SMILES: COc1cc(-c2cn(C)c(=O)c(C)c2C)cc(OC)c1CN1CCN(Cc2cccc3c2CCN(C(=O)/C(C#N)=C/C2(C)CCOCC2)C3C(C)C)C2(CC2)C1] | 4 (in DMA) | 652.7/ 16% |
| | (E/Z)-2-(5-((7-(2,6-dimethoxy-4-(1,4,5-trimethyl-6-oxo-1,6- | | |
| | dihydropyridin-3-yl)benzyl)-4,7-diazaspiro[2.5]octan-4- | | |
| | yl)methyl)-1-isopropyl-1,2,3,4-tetrahydroisoquinoline-2- | | |
| | carbonyl)-3-(4-methyltetrahydro-2H-pyran-4-yl)acrylonitrile | | |
| C24 | 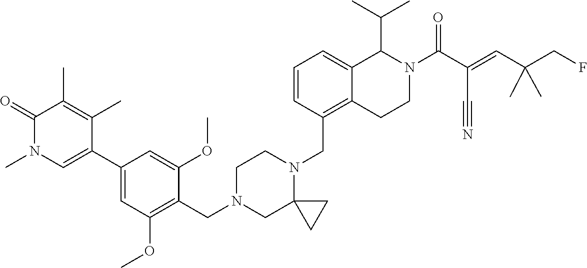 [SMILES: COc1cc(-c2cn(C)c(=O)c(C)c2C)cc(OC)c1CN1CCN(Cc2cccc3c2CCN(C(=O)/C(C#N)=C/C(C)(C)CF)C3C(C)C)C2(CC2)C1] | 4 (with acetic acid in DMA) | 738.3/ 11% |
| | (E/Z)-2-(5-((7-(2,6-dimethoxy-4-(1,4,5-trimethyl-6-oxo-1,6- | | |
| | dihydropyridin-3-yl)benzyl)-4,7-diazaspiro[2.5]octan-4- | | |
| | yl)methyl)-1-isopropyl-1,2,3,4-tetrahydroisoquinoline-2- | | |
| | carbonyl)-5-fluoro-4,4-dimethylpent-2-enenitrile | | |
| C25 | 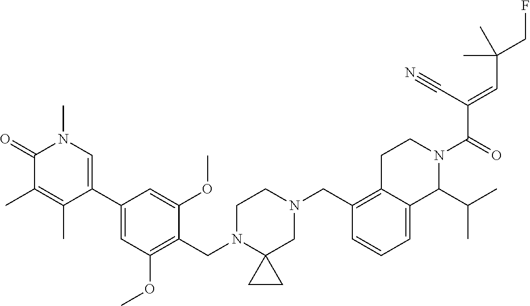 [SMILES: COc1cc(-c2cn(C)c(=O)c(C)c2C)cc(OC)c1CN1CCN(Cc2cccc3c2CCN(C(=O)/C(C#N)=C/C(C)(C)CF)C3C(C)C)CC12CC2] | 4 (in DMF) | 738.4/ 9% |
| | (E/Z)-2-(5-((4-(2,6-dimethoxy-4-(1,4,5-trimethyl-6-oxo-1,6- | | |
| | dihydropyridin-3-yl)benzyl)-4,7-diazaspiro[2.5]octan-7- | | |
| | yl)methyl)-1-isopropyl-1,2,3,4-tetrahydroisoquinoline-2- | | |
| | carbonyl)-5-fluoro-4,4-dimethylpent-2-enenitrile | | |
| C26 | 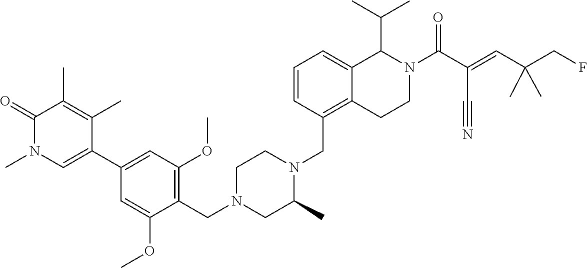 [SMILES: COc1cc(-c2cn(C)c(=O)c(C)c2C)cc(OC)c1CN1CCN(Cc2cccc3c2CCN(C(=O)/C(C#N)=C/C(C)(C)CF)C3C(C)C)[C@@H](C)C1] | 4a | 726.3/ 17% |
| | Molecular Weight: 725.95 | | |
| | (E/Z)-2-(5-(((S)-4-(2,6-dimethoxy-4-(1,4,5-trimethyl-6-oxo- | | |
| | 1,6-dihydropyridin-3-yl)benzyl)-2-methylpiperazin-1- | | |
| | yl)methyl)-1-isopropyl-1,2,3,4-tetrahydroisoquinoline-2- | | |
| | carbonyl)-5-fluoro-4,4-dimethylpent-2-enenitrile | | |

The following analogues were made according to the relevant procedures as noted in the table below, with the following substructure:

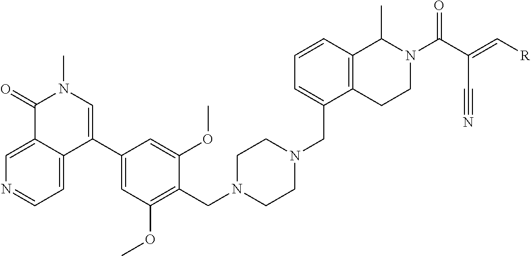

| | | | | LCMS |
|---|---|---|---|---|
| | | | | m/z |
| Name | Name | Procedure | Yield | [M + H]⁺ |
| A26 | (E/Z)-2-(5-((4-(2,6-dimethoxy-4-(2-methyl-1-oxo- | 4 | 9 mg, | 687.0 |
| | 1,2-dihydro-2,7-naphthyridin-4-yl)benzyl)piperazin- | | 5.6% | |
| | 1-yl)methyl)-1-methyl-1,2,3,4- | | | |
| | tetrahydroisoquinoline-2-carbonyl)-3-(1- | | | |
| | methylcyclopropyl)acrylonitrile | | | |
| A22 | (E/Z)-2-(5-((4-(2,6-dimethoxy-4-(2-methyl-1-oxo- | 4 | 40 mg, | 691.0 |
| | 1,2-dihydro-2,7-naphthyridin-4-yl)benzyl)piperazin- | | 21.9% | |
| | 1-yl)methyl)-1-methyl-1,2,3,4- | | | |
| | tetrahydroisoquinoline-2-carbonyl)-3-(1- | | | |
| | fluorocyclopropyl)acrylonitrile | | | |
| A27 | (E/Z)-2-(5-((4-(2,6-dimethoxy-4-(2-methyl-1-oxo- | 4 | 5 mg, | 704.1 |
| | 1,2-dihydro-2,7-naphthyridin-4-yl)benzyl)piperazin- | | 7.2% | |
| | 1-yl)methyl)-1-methyl-1,2,3,4- | | | |
| | tetrahydroisoquinoline-2-carbonyl)-3-(3- | | | |
| | methyloxetan-3-yl)acrylonitrile | | | |
| A24 | (E/Z)-3-cyclobutyl-2-(5-((4-(2,6-dimethoxy-4-(2- | 4 | 22 mg, | 344.2 |
| | methyl-1-oxo-1,2-dihydro-2,7-naphthyridin-4- | | 12.9% | [M + H]²⁺ |
| | yl)benzyl)piperazin-1-yl)methyl)-1-methyl-1,2,3,4- | | | |
| | tetrahydroisoquinoline-2-carbonyl)acrylonitrile | | | |
| A19 | (E/Z)-2-(5-((4-(2,6-dimethoxy-4-(2-methyl-1-oxo- | 4 | 60 mg, | 675.2 |
| | 1,2-dihydro-2,7-naphthyridin-4-yl)benzyl)piperazin- | | 17.6% | |
| | 1-yl)methyl)-1-methyl-1,2,3,4- | | | |
| | tetrahydroisoquinoline-2-carbonyl)-4-methylpent-2- | | | |
| | enenitrile | | | |
| A23 | (E/Z)-2-(5-((4-(2,6-dimethoxy-4-(2-methyl-1-oxo- | 4 | 55 mg, | 716.0 |
| | 1,2-dihydro-2,7-naphthyridin-4-yl)benzyl)piperazin- | | 28.3% | |
| | 1-yl)methyl)-1-methyl-1,2,3,4- | | | |
| | tetrahydroisoquinoline-2-carbonyl)-3-(isothiazol-5- | | | |
| | yl)acrylonitrile | | | |
| A13 | (E/Z)-2-(5-((4-(2,6-dimethoxy-4-(2-methyl-1-oxo- | 4 | 35 mg, | 744.2 |
| | 1,2-dihydro-2,7-naphthyridin-4-yl)benzyl)piperazin- | | 18.2% | |
| | 1-yl)methyl)-1-methyl-1,2,3,4- | | | |
| | tetrahydroisoquinoline-2-carbonyl)-3-(4,5- | | | |
| | dimethylthiazol-2-yl)acrylonitrile | | | |
| A9 | (E/Z)-2-(5-((4-(2,6-dimethoxy-4-(2-methyl-1-oxo- | 4 | 65 mg, | 730.1 |
| | 1,2-dihydro-2,7-naphthyridin-4-yl)benzyl)piperazin- | | 36.9% | |
| | 1-yl)methyl)-1-methyl-1,2,3,4- | | | |
| | tetrahydroisoquinoline-2-carbonyl)-3-(4- | | | |
| | methylthiazol-2-yl)acrylonitrile | | | |
| A10 | (E/Z)-2-(5-((4-(2,6-dimethoxy-4-(2-methyl-1-oxo- | 4 | 40 mg, | 730.1 |
| | 1,2-dihydro-2,7-naphthyridin-4-yl)benzyl)piperazin- | | 21.6% | |
| | 1-yl)methyl)-1-methyl-1,2,3,4- | | | |
| | tetrahydroisoquinoline-2-carbonyl)-3-(5- | | | |
| | methylthiazol-2-yl)acrylonitrile | | | |
| A11 | (E/Z)-2-(5-((4-(2,6-dimethoxy-4-(2-methyl-1-oxo- | 4 | 24 mg, | 700.0 |
| | 1,2-dihydro-2,7-naphthyridin-4-yl)benzyl)piperazin- | | 13.6% | |
| | 1-yl)methyl)-1-methyl-1,2,3,4- | | | |
| | tetrahydroisoquinoline-2-carbonyl)-3-(isoxazol-3- | | | |
| | yl)acrylonitrile | | | |
| A12 | (E/Z)-2-(5-((4-(2,6-dimethoxy-4-(2-methyl-1-oxo- | 4 | 8 mg, | 728.0 |
| | 1,2-dihydro-2,7-naphthyridin-4-yl)benzyl)piperazin- | | 4.1% | |
| | 1-yl)methyl)-1-methyl-1,2,3,4- | | | |
| | tetrahydroisoquinoline-2-carbonyl)-3-(5- | | | |
| | fluoropyridin-2-yl)acrylonitrile | | | |
| A17 | (E/Z)-2-(5-((4-(2,6-dimethoxy-4-(2-methyl-1-oxo- | 4 | 19 mg, | 717.1 |
| | 1,2-dihydro-2,7-naphthyridin-4-yl)benzyl)piperazin- | | 10.2% | |
| | 1-yl)methyl)-1-methyl-1,2,3,4- | | | |
| | tetrahydroisoquinoline-2-carbonyl)-3-(tetrahydro- | | | |
| | 2H-pyran-4-yl)acrylonitrile | | | |
| A18 | (E/Z)-2-(5-((4-(2,6-dimethoxy-4-(2-methyl-1-oxo- | 4 | 22 mg, | 707.3 |
| | 1,2-dihydro-2,7-naphthyridin-4-yl)benzyl)piperazin- | | 9.4% | |
| | 1-yl)methyl)-1-methyl-1,2,3,4- | | | |
| | tetrahydroisoquinoline-2-carbonyl)-5-fluoro-4,4- | | | |
| | dimethylpent-2-enenitrile | | | |
| A2 | (E/Z)-2-(5-((4-(2,6-dimethoxy-4-(2-methyl-1-oxo- | 4 | 80 mg, | 716.0 |
| | 1,2-dihydro-2,7-naphthyridin-4-yl)benzyl)piperazin- | | 45% | |
| | 1-yl)methyl)-1-methyl-1,2,3,4- | | | |
| | tetrahydroisoquinoline-2-carbonyl)-3-(thiazol-2- | | | |
| | yl)acrylonitrile | | | |
| A5 | (E/Z)-3-(6-bromopyridin-2-yl)-2-(5-((4-(2,6- | 4 | 130 | 790.1 |
| | dimethoxy-4-(2-methyl-1-oxo-1,2-dihydro-2,7- | | mg, | |
| | naphthyridin-4-yl)benzyl)piperazin-1-yl)methyl)-1- | | 53.6% | |
| | methyl-1,2,3,4-tetrahydroisoquinoline-2- | | | |
| | carbonyl)acrylonitrile | | | |
| A6 | (E/Z)-3-(1,3-benzothiazol-2-yl)-2-[5-[[4-[[2,6- | 4 | 13 mg, | 766.6 |
| | dimethoxy-4-(2-methyl-1-oxo-2,7-naphthyridin-4- | | 3.1% | |
| | yl)phenylmethyl]piperazin-1-yl]methyl]-1-methyl- | | | |
| | 3,4-dihydro-1H-isoquinoline-2-carbonyl]prop-2- | | | |
| | enenitrile | | | |
| A42 | (E/Z)-2-[5-[[4-[[2,6-dimethoxy-4-(2-methyl-1-oxo- | 4 | 59 mg, | 728.2 |
| | 2,7-naphthyridin-4-yl)phenyl]methyl]piperazin-1- | | 24.9% | |
| | yl]methyl]-1-methyl-3,4-dihydro-1H-isoquinoline-2- | | | |
| | carbonyl]-3-(3-fluoro-2-pyridyl)prop-2-enenitrile | | | |
| A35 | (E/Z)-3-(2,6-difluorophenyl)-2-[5-[[4-[[2,6- | 4 | 65 mg, | 745.2 |
| | dimethoxy-4-(2-methyl-1-oxo-2,7-naphthyridin-4- | | 26.3% | |
| | yl)phenyl]methyl]piperazin-1-yl]methyl]-1-methyl- | | | |
| | 3,4-dihydro-1H-isoquinoline-2-carbonyl]prop-2- | | | |
| | enenitrile | | | |
| A3 | (E/Z)-2-[5-[4-[[2,6-dimethoxy-4-(2-methyl-1-oxo- | 4 | 13.2 | 689.8 |
| | 2,7-naphthyridin-4-yl)phenyl]methyl]piperazin-1- | | mg, | |
| | yl]methyl]-1-methyl-3,4-dihydro-1H-isoquinoline-2- | | 61.5% | |
| | carbonyl]-4,4-dimethyl-pent-2-enenitrile | | | |

The following analogues were made from the relevant beta-cyanoamide according to the relevant procedures as noted in the table below, with the following substructure:

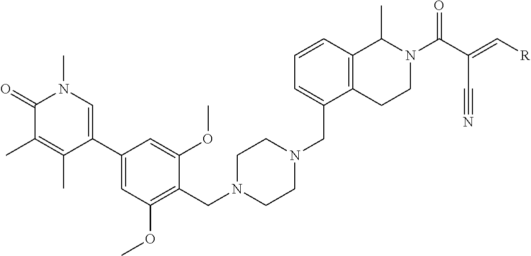

| | | | | LCMS |
|---|---|---|---|---|
| | | | | m/z |
| Name | Name | Procedure | Yield | [M + H]⁺ |
| A16 | (E/Z)-3-(6-bromopyridin-2-yl)-2-(5-((4-(2,6- | 4 | 24 mg, | 766.9 |
| | dimethoxy-4-(1,4,5-trimethyl-6-oxo-1,6- | | 18.7% | |
| | dihydropyridin-3-yl)benzyl)piperazin-1-yl)methyl)- | | | |
| | 1-methyl-1,2,3,4-tetrahydroisoquinoline-2-carbonyl) | | | |
| | acrylonitrile | | | |
| A4 | (E/Z)-2-(5-((4-(2,6-dimethoxy-4-(1,4,5-trimethyl-6- | 4 | 60 mg, | 693.0 |
| | oxo-1,6-dihydropyridin-3-yl)benzyl)piperazin-1- | | 29.8% | |
| | yl)methyl)-1-methyl-1,2,3,4-tetrahydroisoquinoline- | | | |
| | 2-carbonyl)-3-(thiazol-2-yl)acrylonitrile | | | |
| A53 | (E/Z)-2-(5-((4-(2,6-dimethoxy-4-(1,4,5-trimethyl-6- | 4 | 15 mg, | 755.2 |
| | oxo-1,6-dihydropyridin-3-yl)benzyl)piperazin-1- | | 4.4% | |
| | yl)methyl)-1-methyl-1,2,3,4-tetrahydroisoquinoline- | | | |
| | 2-carbonyl)-3-(6-(trifluoromethyl)pyridin-2- | | | |
| | yl)acrylonitrile | | | |
| A67 | (E/Z)-2-(5-((4-(2,6-dimethoxy-4-(1,4,5-trimethyl-6- | 4 | 45 mg, | 717.3 |
| | oxo-1,6-dihydropyridin-3-yl)benzyl)piperazin-1-yl) | | 28.6% | |
| | methyl)-1-methyl-1,2,3,4-tetrahydroisoquinoline-2- | | | |
| | carbonyl)-3-(6-methoxypyridin-2-yl)acrylonitrile | | | |
| A54 | (E/Z)-2-(5-((4-(2,6-dimethoxy-4-(1,4,5-trimethyl-6- | 4 | 40 mg, | 755.2 |
| | oxo-1,6-dihydropyridin-3-yl)benzyl)piperazin-1- | | 12.4% | |
| | yl)methyl)-1-methyl-1,2,3,4-tetrahydroisoquinoline- | | | |
| | 2-carbonyl)-3-(5-(trifluoromethyl)pyridin-2- | | | |
| | yl)acrylonitrile | | | |
| A20 | (E/Z)-3-(1,3-benzothiazol-2-yl)-2-[5-[4-[2,6- | 4 | 10 mg, | 743.4 |
| | dimethoxy-4-(1,4,5-trimethyl-6-oxo-3- | | 6.5% | |
| | pyridyl)phenyl]methyl]piperazin-1-yl]methyl]-1- | | | |
| | methyl-3,4-dihydro-1H-isoquinoline-2- | | | |
| | carbonyl]prop-2-enenitrile | | | |
| A21 | (E/Z)-2-[5-[4-[[2,6-dimethoxy-4-(1,4,5-trimethyl-6- | 4 | 50 mg, | 707.1 |
| | oxo-3-pyridyl)phenylmethyl]piperazin-1-yl]]methyl]- | | 29.0% | |
| | 1-methyl-3,4-dihydro-1H-isoquinoline-2-carbonyl]-3- | | | |
| | (4-methylthiazol-2-yl)prop-2-enenitrile | | | |
| A29 | (E/Z)-2-[5-[4-[2,6-dimethoxy-4-(1,4,5-trimethyl-6- | 4 | 60 mg, | 707.2 |
| | oxo-3-pyridyl)phenylmethyl]piperazin-1-yl]]methyl]- | | 33.4% | |
| | 1-methyl-3,4-dihydro-1H-isoquinoline-2-carbonyl]-3- | | | |
| | (5-methylthiazol-2-yl)prop-2-enenitrile | | | |
| A51 | (E/Z)-2-[5-[[4-[[2,6-dimethoxy-4-(1,4,5-trimethyl-6- | 4 | 12 mg, | 680.2 |
| | oxo-3-pyridyl)phenyl]methyl]piperazin-1-yl]methyl]- | | 6.8% | |
| | 1-methyl-3,4-dihydro-1H-isoquinoline-2-carbonyl]-3- | | | |
| | (3-methyloxetan-3-yl)prop-2-enenitrile | | | |
| A45 | (E/Z)-3-cyclobutyl-2-[5-[4-[2,6-dimethoxy-4-(1,4,5- | 4 | 22 mg, | 664.0 |
| | trimethyl-6-oxo-3-pyridyl)phenyl]methyl]piperazin-1- | | 7.9% | |
| | yl]methyl]-1-methyl-3,4-dihydro-1H-isoquinoline-2- | | | |
| | carbonyl]prop-2-enenitrile | | | |
| A43 | (E/Z)-2-[5-[[4-[2,6-dimethoxy-4-(1,4,5-trimethyl-6- | 4 | 50 mg, | 652.2 |
| | oxo-3-pyridyl)phenyl]methyl]piperazin-1-yl]methyl]- | | 14.5% | |
| | 1-methyl-3,4-dihydro-1H-isoquinoline-2-carbonyl]-4- | | | |
| | methyl-pent-2-enenitrile | | | |
| A44 | (E/Z)-3-cyclopropyl-2-[5-[[4-[2,6-dimethoxy-4- | 4 | 80 mg, | 650.2 |
| | (1,4,5-trimethyl-6-oxo-3- | | 23.8% | |
| | pyridyl)phenyl]methyl]piperazin-1-yl]methyl]-1- | | | |
| | methyl-3,4-dihydro-1H-isoquinoline-2- | | | |
| | carbonyl]prop-2-enenitrile | | | |
| A7 | (E/Z)-2-(5-((4-(2,6-dimethoxy-4-(1,4,5-trimethyl-6- | 4 | 16 mg, | 666.8 |
| | oxo-1,6-dihydropyridin-3-yl)benzyl)piperazin-1- | | 37.8% | |
| | yl)methyl)-1-methyl-1,2,3,4-tetrahydroisoquinoline- | | | |
| | 2-carbonyl)-4,4-dimethylpent-2-enenitrile | | | |
| A8 | (E)-2-(5-((4-(2,6-dimethoxy-4-(1,4,5-trimethyl-6-oxo- | 4 | 25 mg, | 690.8 |
| | 1,6-dihydropyridin-3-yl)benzyl)piperazin-1- | | 55.3% | |
| | yl)methyl)-1-methyl-1,2,3,4-tetrahydroisoquinoline- | | | |
| | 2-carbonyl)-3-(1-methyl-1H-pyrazol-3-yl)acrylonitrile | | | |
| B70 | (E/Z)-2-(5-((4-(2,6-dimethoxy-4-(1,4,5-trimethyl-6- | 4 | 7 mg, | 708.4 |
| | oxo-1,6-dihydropyridin-3-yl)benzyl)piperazin-1- | | 28.0% | |
| | yl)methyl)-1-methyl-1,2,3,4-tetrahydroisoquinoline- | | | |
| | 2-carbonyl)-3-(2-methoxypyridin-3-yl)acrylonitrile | | | |
| B69 | (E/Z)-2-(5-((4-(2,6-dimethoxy-4-(1,4,5-trimethyl-6- | 4 | 3 mg, | 694.3 |
| | oxo-1,6-dihydropyridin-3-yl)benzyl)piperazin-1- | | 12% | |
| | yl)methyl)-1-methyl-1,2,3,4-tetrahydroisoquinoline- | | | |
| | 2-carbonyl)-3-(tetrahydro-2H-pyran-4- | | | |
| | yl)acrylonitrile | | | |
| B1 | (E/Z)-3-(3,3-difluorocyclobutyl)-2-(5-((4-(2,6- | 4 | 9 mg, | 700.3 |
| | dimethoxy-4-(1,4,5-trimethyl-6-oxo-1,6- | | 3% | |
| | dihydropyridin-3-yl)benzyl)piperazin-1-yl)methyl)-1- | | | |
| | methyl-1,2,3,4-tetrahydroisoquinoline-2- | | | |
| | carbonyl)acrylonitrile | | | |
| B71 | (E/Z)-2-(5-((4-(2,6-dimethoxy-4-(1,4,5-trimethyl-6- | 4 | 9.5 mg, | 717.3 |
| | ox0-1,6-dihydropyridin-3-yl)benzyl)piperazin-1- | | 26% | |
| | yl)methyl)-1-methyl-1,2,3,4-tetrahydroisoquinoline- | | | |
| | 2-carbonyl)-3-(6-methoxypyridin-3-yl)acrylonitrile | | | |
| B72 | (E/Z)-3-(4,4-difluorocyclohexyl)-2-(5-((4-(2,6- | 4 | 4 mg, | 728.3 |
| | dimethoxy-4-(1,4,5-trimethyl-6-oxo-1,6- | | 10% | |
| | dihydropyridin-3-yl)benzyl)piperazin-1-yl)methyl)-1- | | | |
| | methyl-1,2,3,4-tetrahydroisoquinoline-2- | | | |
| | carbonyl)acrylonitrile | | | |
| B126 | (E/Z)-2-(5-((7-(2,6-dimethoxy-4-(1,4,5-trimethyl-6- | 4 | 89 mg, | 818.4 |
| | oxo-1,6-dihydropyridin-3-yl)benzyl)-4,7- | | 45% | |
| | diazaspiro[2.5]octan-4-yl)methyl)-1-methyl-1,2,3,4- | | | |
| | tetrahydroisoquinoline-2-carbonyl)-4-methyl-4-(4- | | | |
| | (oxetan-3-yl)piperazin-1-yl)pent-2-enenitrile | | | |
| B73 | (E/Z)-3-cyclohexyl-2-(5-((4-(2,6-dimethoxy-4-(1,4,5- | 4a | 5.7 mg, | 692.4 |
| | trimethyl-6-oxo-1,6-dihydropyridin-3- | | 16% | |
| | yl)benzyl)piperazin-1-yl)methyl)-1-methyl-1,2,3,4- | | | |
| | tetrahydroisoquinoline-2-carbonyl)acrylonitrile | | | |
| B74 | (E/Z)-3-(2-cyano-3-(5-((4-(2,6-dimethoxy-4-(1,4,5- | 4a | 5 mg, | 711.3 |
| | trimethyl-6-oxo-1,6-dihydropyridin-3- | | 11% | |
| | yl)benzyl)piperazin-1-yl)methyl)-1-methyl-3,4- | | | |
| | dihydroisoquinolin-2(1H)-yl)-3-oxoprop-1-en-1-yl) | | | |
| | benzonitrile | | | |
| B76 | (E/Z)-2-(5-((4-(2,6-dimethoxy-4-(1,4,5-trimethyl-6- | 4a (see | 8.8 mg, | 768.4 |
| | oxo-1,6-dihydropyridin-3-yl)benzyl)piperazin-1- | WO2007/6 | 24% | |
| | yl)methyl)-1-methyl-1,2,3,4-tetrahydroisoquinoline- | 716 for | | |
| | 2-carbonyl)-3-(1-methylcyclobutyl)acrylonitrile | preparation | | |
| | | of | | |
| | | aldehyde) | | |
| B90 | (E/Z)-3-cyclopentyl-2-(5-((4-(2,6-dimethoxy-4- | 4 | 5.3 mg, | 678.4 |
| | (1,4,5-trimethyl-6-oxo-1,6-dihydropyridin-3- | (methanol) | 16% | |
| | yl)benzyl)piperazin-1-yl)methyl)-1-methyl-1,2,3,4- | | | |
| | tetrahydroisoquinoline-2-carbonyl)acrylonitrile | | | |
| B89 | (E/Z)-2-(5-((4-(2,6-dimethoxy-4-(1,4,5-trimethyl-6- | 4 | 9 mg, | 702.3 |
| | oxo-1,6-dihydropyridin-3-yl)benzyl)piperazin-1- | (methanol) | 3% | |
| | yl)methyl)-1-methyl-1,2,3,4-tetrahydroisoquinoline- | | | |
| | 2-carbonyl)-3-(5-methylpyrazin-2-yl)acrylonitrile | | | |
| B94 | (E/Z)-2-(5-((4-(2,6-dimethoxy-4-(1,4,5-trimethyl-6- | 4a | 3.1 mg, | 598.4 |
| | oxo-1,6-dihydropyridin-3-yl)benzyl)piperazin-1- | | 9% | |
| | yl)methyl)-1-methyl-1,2,3,4-tetrahydroisoquinoline- | | | |
| | 2-carbonyl)-3-(tetrahydrofuran-3-yl)acrylonitrile | | | |
| B116 | (E/Z)-3-(3,3-difluorocyclobutyl)-2-(5-((4-(2,6- | 4 | 2.8 mg, | 726.3 |
| | dimethoxy-4-(1,4,5-trimethyl-6-oxo-1,6- | | 4.7% | |
| | dihydropyridin-3-yl)benzyl)piperazin-1-yl)methyl)-1- | | | |
| | methyl-1,2,3,4-tetrahydroisoquinoline-2- | | | |
| | carbonyl)acrylonitrile | | | |
| B9 | (E/Z)-2-(5-((4-(2,6-dimethoxy-4-(1,4,5-trimethyl-6- | 4a | 20 mg, | 702.3 |
| | oxo-1,6-dihydropyridin-3-yl)benzyl)piperazin-1- | | 11% | |
| | yl)methyl)-1-methyl-1,2,3,4-tetrahydroisoquinoline- | | | |
| | 2-carbonyl)-3-(2-methylpyrimidin-4-yl)acrylonitrile | | | |
| B10 | (E/Z)-2-(5-((4-(2,6-dimethoxy-4-(1,4,5-trimethyl-6- | 17a | 30 mg, | 761.2 |
| | oxo-1,6-dihydropyridin-3-yl)benzyl)piperazin-1- | | 23.3% | |
| | yl)methyl)-1-methyl-1,2,3,4-tetrahydroisoquinoline- | | | |
| | 2-carbonyl)-3-(5-(trifluoromethyl)thiazol-2- | | | |
| | yl)acrylonitrile | | | |
| B11 | (E/Z)-2-(5-((4-(2,6-dimethoxy-4-(1,4,5-trimethyl-6- | 17a | 16 mg, | 761.2 |
| | oxo-1,6-dihydropyridin-3-yl)benzyl)piperazin-1- | | 12.5% | |
| | yl)methyl)-1-methyl-1,2,3,4-tetrahydroisoquinoline- | | | |
| | 2-carbonyl)-3-(4-(trifluoromethyl)thiazol-2- | | | |
| | yl)acrylonitrile | | | |
| B24 | (E/Z)-2-(5-((4-(2,6-dimethoxy-4-(1,4,5-trimethyl-6- | 4a | 43 mg, | 701.1 |
| | oxo-1,6-dihydropyridin-3-yl)benzyl)piperazin-1- | | 23.8% | |
| | yl)methyl)-1-methyl-1,2,3,4-tetrahydroisoquinoline- | | | |
| | 2-carbonyl)-3-(6-methylpyridin-2-yl)acrylonitrile | | | |
| B4 | (E/Z)-2-(5-((4-(2,6-dimethoxy-4-(1,4,5-trimethyl-6- | 4a | 70 mg, | 691.3 |
| | oxo-1,6-dihydropyridin-3-yl)benzyl)piperazin-1- | | 43.3 | |
| | yl)methyl)-1-methyl-1,2,3,4-tetrahydroisoquinoline- | | | |
| | 2-carbonyl)-3-(5-methylisoxazol-3-yl)acrylonitrile | | | |
| B52 | (E/Z)-2-(5-((4-(2,6-dimethoxy-4-(1,4,5-trimethyl-6- | 17d | 31 mg, | 717.2 |
| | oxo-1,6-dihydropyridin-3-yl)benzyl)piperazin-1- | | 16% | |
| | yl)methyl)-1-methyl-1,2,3,4-tetrahydroisoquinoline- | | | |
| | 2-carbonyl)-3-(4-methoxypyridin-2-yl)acrylonitrile | | | |
| B49 | (E/Z)-2-(5-((4-(2,6-dimethoxy-4-(1,4,5-trimethyl-6- | 17d | 29 mg, | 755.2 |
| | oxo-1,6-dihydropyridin-3-yl)benzyl)piperazin-1- | | 16% | |
| | yl)methyl)-1-methyl-1,2,3,4-tetrahydroisoquinoline- | | | |
| | 2-carbonyl)-3-(3-(trifluoromethyl)pyridin-2- | | | |
| | yl)acrylonitrile | | | |
| B53 | (E/Z)-2-(5-((4-(2,6-dimethoxy-4-(1,4,5-trimethyl-6- | 17d | 27 mg, | 717.2 |
| | oxo-1,6-dihydropyridin-3-yl)benzyl)piperazin-1- | | 14% | |
| | yl)methyl)-1-methyl-1,2,3,4-tetrahydroisoquinoline- | | | |
| | 2-carbonyl)-3-(5-methoxypyridin-2-yl)acrylonitrile | | | |
| B36 | (E/Z)-2-(5-((4-(2,6-dimethoxy-4-(1,4,5-trimethyl-6- | 17a | 14 mg, | 732.4 |
| | oxo-1,6-dihydropyridin-3-yl)benzyl)piperazin-1- | | 22% | |
| | yl)methyl)-1-methyl-1,2,3,4-tetrahydroisoquinoline- | | | |
| | 2-carbonyl)-3-(1- | | | |
| | (trifluoromethyl)cyclobutyl)acrylonitrile | | | |
| B18 | (E/Z)-2-(5-((4-(2,6-dimethoxy-4-(1,4,5-trimethyl-6- | 4 | 50 mg, | 701.4 |
| | oxo-1,6-dihydropyridin-3-yl)benzyl)piperazin-1- | | 15% | |
| | yl)methyl)-1-methyl-1,2,3,4-tetrahydroisoquinoline- | | | |
| | 2-carbonyl)-3-(5-methylpyridin-2-yl)acrylonitrile | | | |
| B19 | (E/Z)-2-(5-((4-(2,6-dimethoxy-4-(1,4,5-trimethyl-6- | 4 | 50 mg, | 701.4 |
| | oxo-1,6-dihydropyridin-3-yl)benzyl)piperazin-1- | | 15% | |
| | yl)methyl)-1-methyl-1,2,3,4-tetrahydroisoquinoline- | | | |
| | 2-carbonyl)-3-(4-methylpyridin-2-yl)acrylonitrile | | | |
| B20 | (E/Z)-2-(5-((4-(2,6-dimethoxy-4-(1,4,5-trimethyl-6- | 4 (see | 11 mg, | 668.2 |
| | oxo-1,6-dihydropyridin-3-yl)benzyl)piperazin-1- | WO2020/ | 7.8% | |
| | yl)methyl)-1-methyl-1,2,3,4-tetrahydroisoquinoline- | 146194 for | | |
| | 2-carbonyl)-3-(1-fluorocyclopropyl)acrylonitrile | preparation | | |
| | | of | | |
| | | aldehyde) | | |
| B45 | (E/Z)-3-(bicyclo[1.1.1]pentan-1-yl)-2-(5-((4-(2,6- | 17c (see | 50 mg, | 676.3 |
| | dimethoxy-4-(1,4,5-trimethyl-6-oxo-1,6- | US2018/ | 43% | |
| | dihydropyridin-3-yl)benzyl)piperazin-1-yl)methyl)-1- | 99931 for | | |
| | methyl-1,2,3,4-tetrahydroisoquinoline-2- | preparation | | |
| | carbonyl)acrylonitrile | of | | |
| | | aldehyde) | | |
| B50 | (E/Z)-2-(5-((4-(2,6-dimethoxy-4-(1,4,5-trimethyl-6- | 17c | 28 mg, | 755.2 |
| | oxo-1,6-dihydropyridin-3-yl)benzyl)piperazin-1- | | 14% | |
| | yl)methyl)-1-methyl-1,2,3,4-tetrahydroisoquinoline- | | | |
| | 2-carbonyl)-3-(4-(trifluoromethyl)pyridin-2- | | | |
| | yl)acrylonitrile: | | | |
| B51 | (E/Z)-2-(5-((4-(2,6-dimethoxy-4-(1,4,5-trimethyl-6- | 4a | 73 mg, | 717.2 |
| | oxo-1,6-dihydropyridin-3-yl)benzyl)piperazin-1- | | 40% | |
| | yl)methyl)-1-methyl-1,2,3,4-tetrahydroisoquinoline- | | | |
| | 2-carbonyl)-3-(3-methoxypyridin-2-yl)acrylonitrile | | | |
| B91 | (E/Z)-3-(2,3-difluorophenyl)-2-(5-((4-(2,6-dimethoxy- | 4a | 3.7 mg, | 722.3 |
| | 4-(1,4,5-trimethyl-6-oxo-1,6-dihydropyridin-3- | | 10% | |
| | yl)benzyl)piperazin-1-yl)methyl)-1-methyl-1,2,3,4- | | | |
| | tetrahydroisoquinoline-2-carbonyl)acrylonitrile | | | |
| B92 | (E/Z)-2-(5-((4-(2,6-dimethoxy-4-(1,4,5-trimethyl-6- | 4a | 5.2 mg, | 735.3 |
| | oxo-1,6-dihydropyridin-3-yl)benzyl)piperazin-1- | | 14% | |
| | yl)methyl)-1-methyl-1,2,3,4-tetrahydroisoquinoline- | | | |
| | 2-carbonyl)-3-(5-fluoro-2-methoxypyridin-4- | | | |
| | yl)acrylonitrile | | | |
| B93 | (E/Z)-2-(5-((4-(2,6-dimethoxy-4-(1,4,5-trimethyl-6- | 4a | 9 mg, | 734.3 |
| | oxo-1,6-dihydropyridin-3-yl)benzyl)piperazin-1- | | 24% | |
| | yl)methyl)-1-methyl-1,2,3,4-tetrahydroisoquinoline- | | | |
| | 2-carbonyl)-3-(2-fluoro-5- | | | |
| | methoxyphenyl)acrylonitrile | | | |
| B75 | (E/Z)-2-(5-((4-(2,6-dimethoxy-4-(1,4,5-trimethyl-6- | 4a | 5.6 mg, | 735.2 |
| | oxo-1,6-dihydropyridin-3-yl)benzyl)piperazin-1- | | 15% | |
| | yl)methyl)-1-methyl-1,2,3,4-tetrahydroisoquinoline- | | | |
| | 2-carbonyl)-3-(2-fluoro-5-methoxypyridin-3- | | | |
| | yl)acrylonitrile | | | |
| C27 | (E/Z)-2-(5-((4-(2,6-dimethoxy-4-(1,4,5-trimethyl-6- | 4a | 22 mg, | 664.4 |
| | oxo-1,6-dihydropyridin-3-yl)benzyl)piperazin-1- | | 10% | |
| | yl)methyl)-1-methyl-1,2,3,4-tetrahydroisoquinoline- | | | |
| | 2-carbonyl)-3-(1-methylcyclopropyl)acrylonitrile | | | |

### Intermediate W-29 and W-30

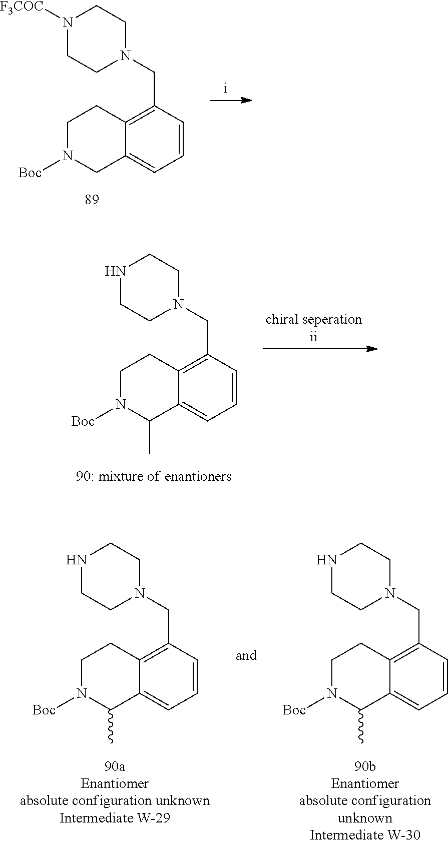

89: By general procedure 1 from 2,2,2-trifluoro-1-(piperazin-1-yl)ethan-1-one (WO02010/124082) with MP-CNBH₃ (1 equiv.). Obtained 6.5 g, 66.9% yield. LCMS m/z=442.1 [M+H]⁺.

90: By general procedure 20. Crude was diluted with 15% MeOH in DCM. The organic phase was extracted with water then dried over anhydrous sodium sulphate and concentrated in vacuo. Obtained 5.5 g, 93%. LCMS m/z=346.2 [M+H]⁺.

### 90a (Enantiomer 1) and 90b (Enantiomer 2): SFC Chiral Separation Method—Absolute Configuration Unknown.

Instrument: PIC 175: Column: LUX-C4; Mobile phase: 60:40 CO₂: 0.5% isopropylamine in IPA; Total flow: 5 mL/min; Back pressure: 100 bar; Wavelength: 210 nm; Cycle time: 6 min

### Compound A15: Enantiomer 1 of (E/Z)-2-(5((4-(2,6-dimethoxy-4-(2-methyl-1-oxo-1,2-dihydro-2,7-naphthyridin-4-yl)benzyl)piperazin-1-yl)methyl)-1-methyl-1,2,3,4-tetrahydroisoquinoline-2-carbonyl)-4-thiazol-2-yl)acrylonitrile

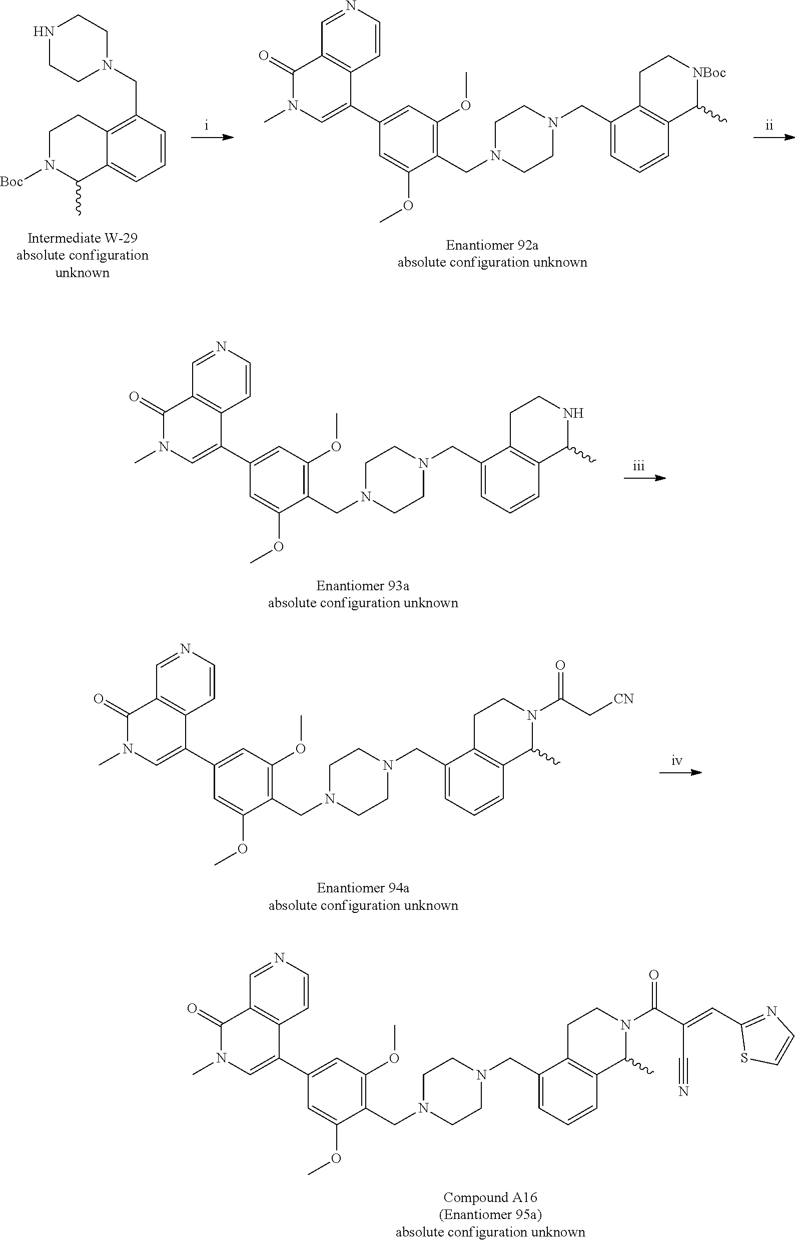

92a: By general procedure 1 with MP-CNBH₃ (1 equiv.). Obtained 1.3 g, 75% yield. LCMS m/z=654.1 [M+H]⁺.

93a: By general procedure 2. Obtained 1.1 g, 99% yield. LCMS m/z=554.1 [M+H]⁺.

94a: By general procedure 3. Obtained 950 mg, 54.2% yield. LCMS m/z=621.1 [M+H]⁺.

Compound A15: By general procedure 4. Obtained 39 mg, 26.3% yield. LCMS m/z=716.0 [M+H]⁺.

### Compounds A14 and A25

### A14: Enantiomer 2 of (E/Z)-2-(5-((4-(2,6-dimethoxy-4-(2-methyl-1-oxo-1,2-dihydro-2,7-naphthyridin-4-yl)benzyl)piperazin-1-yl)methyl)-1-methyl-1,2,3,4-tetrahydroisoquinoline-2-carbonyl)-3-(thiazol-2-yl)acrylonitrile

### A25: Enantiomer 2 of (E/Z)-2-(5-((4-(2,6-dimethoxy-4-(2-methyl-1-oxo-1,2-dihydro-2,7-naphthyridin-4-yl)benzyl)piperazin-1-yl)methyl)-1-methyl-1,2,3,4-tetrahydroisoquinoline-2-carbonyl)-4,4-dimethylpent-2-enenitrile

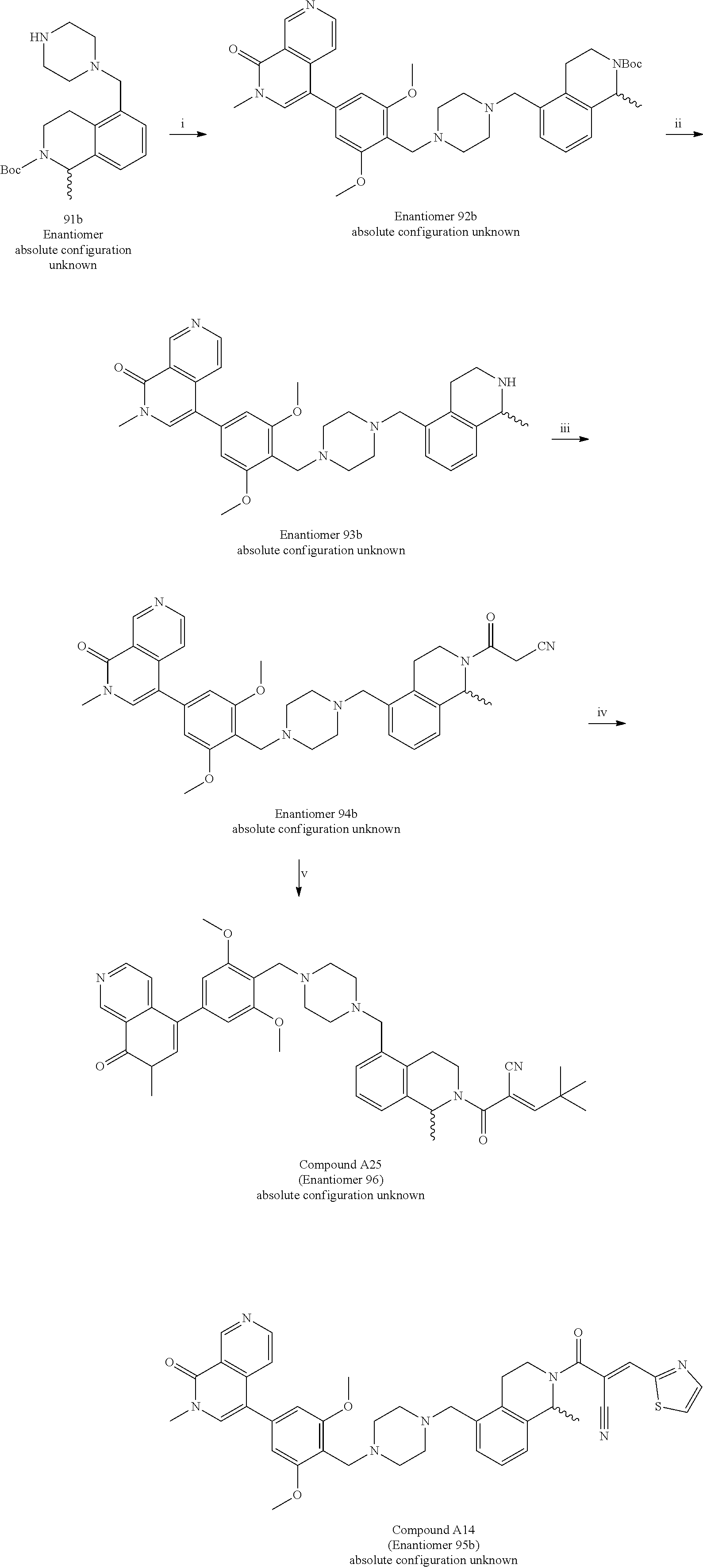

92b: By general procedure 1 with MP-CNBH₃ (1 equiv.). Obtained 1.1 g, 97% yield. LCMS m/z=654.0 [M+H]⁺.

93b: By general procedure 2. Obtained 0.9 g, 98% yield. LCMS m/z=554.1 [M+H]⁺.

94b: By general procedure 3. Obtained 800 mg, 48.2% yield. LCMS m/z=621.1 [M+H]⁺.

Compound A14: By general procedure 4. Obtained 55 mg, 47.7% yield. LCMS m/z=716.2 [M+H]⁺.

Compound A25: By general procedure 4. Obtained 12 mg, 36.4% yield. LCMS m/z=689.2 [M+H]⁺

### Compound A30: 2-(6-((1-(2,6-dimethoxy-4-(2-methyl-1-oxo-1,2-dihydro-2,7-naphthyridin-4-yl) benzyl) piperidin-4-yl) (methyl) amino)-1-methyl-1,2,3,4-tetrahydroisoquinoline-2-carbonyl)-4,4-dimethylpent-2-enenitrile

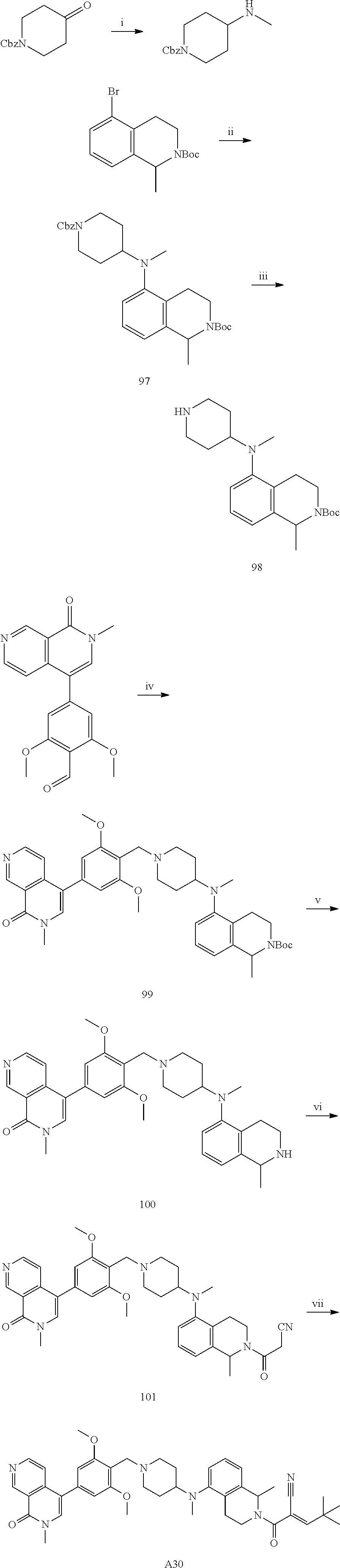

97: To a stirred solution of tert-butyl 5-bromo-1-methyl-3,4-dihydroisoquinoline-2(1H)-carboxylate (1.0 g, 3.22 mmol) and benzyl 4-(methylamino)piperidine-1-carboxylate (prepared as described in WO2009/76387, 1.0 g, 4.02 mmol) in THF (10 mL) was added DavePhos (0.15 g, 0.41 mmol), LiHMDS (2 M, 2 mL, 8.05 mmol) and Pd₂dba₃ (0.25 g, 0.28 mmol) at RT. The reaction mixture was stirred at 55° C. for 18 h then was filtered through celite. The filtrate was concentrated in vacuo and purified by silica gel column chromatography (gradient=50% EtOAc in n-hexane) to afford tert-butyl 5-[(1-benzyloxycarbonyl-4-piperidyl)-methyl-amino]-1-methyl-3,4-dihydro-1H-isoquinoline-2-carboxylate (0.81 g, 1.52 mmol, 37.8% yield). LCMS m/z=494.0 [M+H]⁺.

98: To a stirred solution of tert-butyl 5-[(1-benzyloxycarbonyl-4-piperidyl)-methyl-amino]-1-methyl-3,4-dihydro-1H-isoquinoline-2-carboxylate (0.8 g, 1.52 mmol) in MeOH (10 mL) was added Pd/C (0.11 g, 1.03 mmol) under N₂ atmosphere. The reaction mixture was stirred under H₂ atmosphere at RT for 16 h then was filtered through celite. The filtrate was concentrated in vacuo to afford tert-butyl 1-methyl-5-[methyl(4-piperidyl)amino]-3,4-dihydro-1H-isoquinoline-2-carboxylate (610 mg, 1.35 mmol, 70% yield). LCMS m/z=360.2 [M+H]⁺.

99: By general procedure 1 using MP-CNBH₃. Obtained 800 mg, 59.4% yield. LCMS m/z=688.0 [M+H]⁺.

100: By general procedure 2. Obtained 400 mg, 88% yield. LCMS m/z=588.3 [M+H]⁺.

101: By general procedure 3. Obtained 250 mg, 53% yield. LCMS m/z=635.3 [M+H]⁺.

Compound A30: By general procedure 4. Obtained 25.9 mg, 28.8% yield. LCMS m/z=703.3 [M+H]⁺.

### Compounds A49 and A65: (E/Z)-3-(6-bromopyridin-2-yl)-2-(5-((8-(2,6-dimethoxy-4-(2-methyl-1-oxo-1,2-dihydro-2,7-naphthyridin-4-yl) benzyl)-3,8-diazabicyclo[3.2.1]octan-3-yl) methyl)-1-methyl-1,2,3,4-tetrahydroisoquinoline-2-carbonyl) acrylonitrile

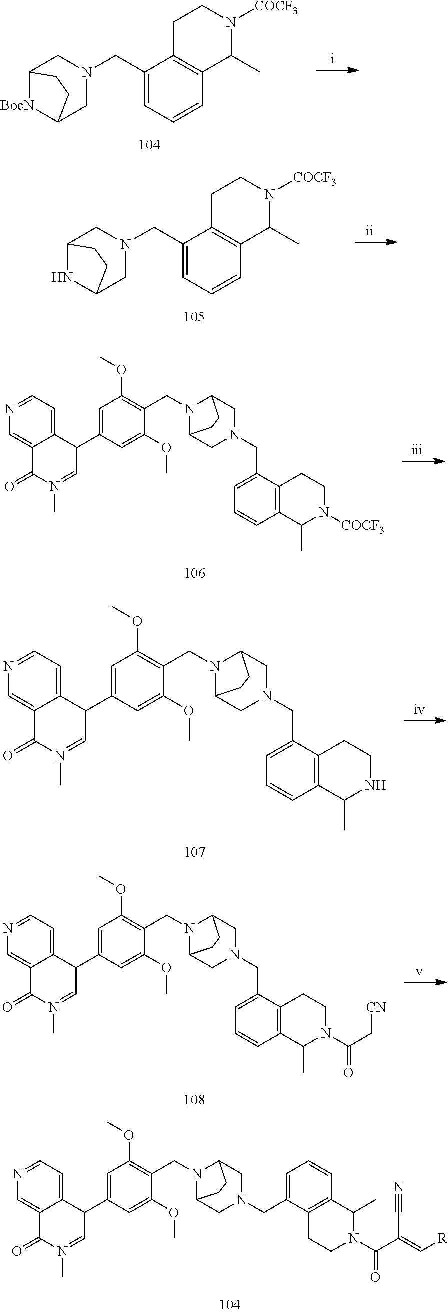

By general procedure 1 using MP-CNBH₃ (1 equiv.). Obtained 800 mg, 91% yield. LCMS m/z=488.2 [M+H]⁺.

105: By general procedure 2. Obtained 690 mg, 93% yield. LCMS m/z=404.5 [M+H]⁺.

106: By general procedure 1 using MP-CNBH₃ (1 equiv.) in DCE. Obtained 470 mg, 37.6% yield. LCMS m/z=676.3 [M+H]⁺.

107: By general procedure 20. The crude was diluted with 15% MeOH in DCM and was washed with water, brine (saturated aqueous solution) and the organic phase was dried over anhydrous sodium sulphate and concentrated in vacuo. Obtained 300 mg, 88% yield. LCMS m/z=580.2 [M+H]⁺.

| | | | | LCMS |
|---|---|---|---|---|
| | | | | m/z |
| Cmpd | Structure | Procedure | Yield | [M + H]⁺ |
| A49 | 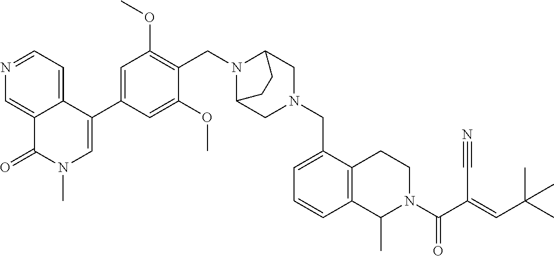 [SMILES: COc1cc(-c2cn(C)c(=O)c3cnccc23)cc(OC)c1CN1C2CCC1CN(Cc1cccc3c1CCN(C(=O)/C(C#N)=C/C(C)(C)C)C3C)C2] | 4 | 35 mg, 25.9% | 715.3 |
| A65 | 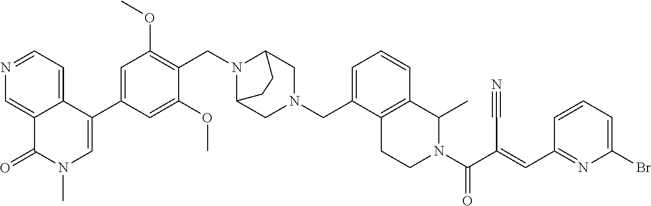 [SMILES: COc1cc(-c2cn(C)c(=O)c3cnccc23)cc(OC)c1CN1C2CCC1CN(Cc1cccc3c1CCN(C(=O)/C(C#N)=C/c1cccc(Br)n1)C3C)C2] | 4 | 2.5 mg, 12.9% yield | 751.1 |

### Compound A58: (E/Z)-2-cyano-N-(1-(4-(3-(4-(2,6-dimethoxy-4-(1,4,5-trimethyl-6-oxo-1,6-dihydropyridin-3-yl)benzyl)piperazin-1-yl)-3-oxopropyl)phenyl)butyl)-N-methyl-3-(thiazol-2-yl)acrylamide

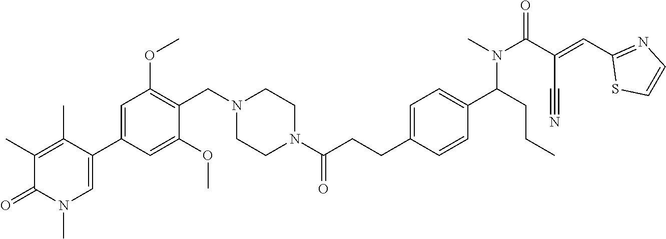 [SMILES: CCCC(c1ccc(CCC(=O)N2CCN(Cc3c(OC)cc(-c4cn(C)c(=O)c(C)c4C)cc3OC)CC2)cc1)N(C)C(=O)/C(C#N)=C/c1nccs1]

Prepared following general procedure 6 from Intermediate W-2 and the amine from Table 4a. Obtained 120 mg, 41.9% yield. LCMS m/z=751.1 [M+H]⁺.

### Compound A57: (E/Z)-2-(5-(3-(4-(2,6-dimethoxy-4-(1,4,5-trimethyl-6-oxo-1,6-dihydropyridin-4-yl) benzyl) piperazin-1-yl)-3-oxopropyl)-1-methyl-1,2,3,4-tetrahydroisoquinoline-2-carbonyl)-3-(thiazol-2-yl)acrylonitrile

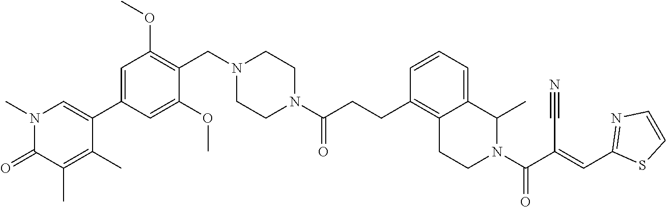 [SMILES: COc1cc(-c2cn(C)c(=O)c(C)c2C)cc(OC)c1CN1CCN(C(=O)CCc2cccc3c2CCN(C(=O)/C(C#N)=C/c2nccs2)C3C)CC1]

Prepared following general procedure 6 from 3-(1-methyl-1,2,3,4-tetrahydroisoquinolin-5-yl)propanoic acid (prepared as described in WO2022/129925). Obtained 99 mg, 32.9% yield. LCMS m/z=735.1 [M−H]⁺.

### Compound A55: (E/Z)-2-(7(3-(4-(2,6-dimethoxy-4-(1,4,5-trimethyl-6-oxo-1,6-dihydropyridin-3-yl) benzyl) piperazin-1-yl)-3-oxopropyl)-1-methyl-1,2,3,4-tetrahydroisoquinoline-2-carbonyl)-3-(thiazol-2-yl)acrylonitrile

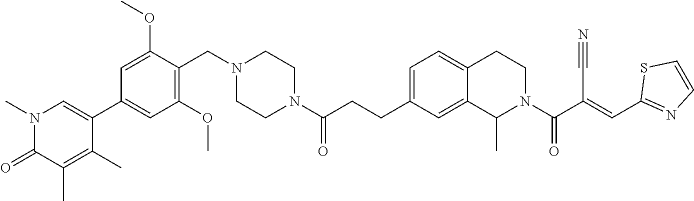 [SMILES: COc1cc(-c2cn(C)c(=O)c(C)c2C)cc(OC)c1CN1CCN(C(=O)CCc2ccc3c(c2)C(C)N(C(=O)/C(C#N)=C/c2nccs2)CC3)CC1]

Prepared following general procedure 6 using (E/Z)-3-(2-(2-cyano-3-(thiazol-2-yl)acryloyl)-1-methyl-1,2,3,4-tetrahydroisoquinolin-7-yl)propanoic acid (Intermediate W-5). Obtained 67 mg, 28.6% yield. LCMS m/z=735.1 [M+H]⁺.

### Compound A56: (E/Z)-2-(6-(3-(4-(2,6-dimethoxy-4-(1,4,5-trimethyl-6-oxo-1,6-dihydropyridin-3-yl) benzyl) piperazin-1-yl)-3-oxopropyl)-1-methyl-1,2,3,4-tetrahydroisoquinoline-2-carbonyl)-3-(thiazol-2-yl)acrylonitrile

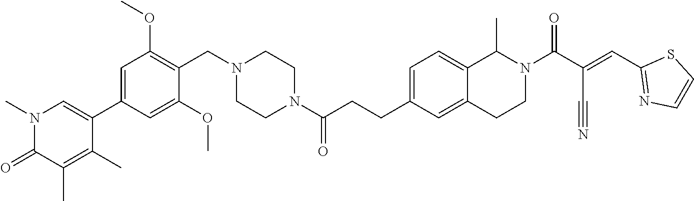 [SMILES: COc1cc(-c2cn(C)c(=O)c(C)c2C)cc(OC)c1CN1CCN(C(=O)CCc2ccc3c(c2)CCN(C(=O)/C(C#N)=C/c2nccs2)C3C)CC1]

Prepared following general procedure 6 from E/Z)-3-(2-(2-cyano-3-(thiazol-2-yl)acryloyl)-1-methyl-1,2,3,4-tetrahydroisoquinolin-8-yl)propanoic acid, prepared as described in WO2022/129925. Obtained 70 mg, 23.9% yield. LCMS m/z=735.1 [M+H]⁺.

### Compounds A68. B44. B64. B22

### A68: (E/Z)-3-(6-bromopyridin-2-yl)-2-(5-((1-(2,6-dimethoxy-4-(1,4,5-trimethyl-6-oxo-1,6-dihydropyridin-3-yl) benzyl) piperidin-4-yl) oxy)-1-methyl-1,2,3,4-tetrahydroisoquinoline-2-carbonyl)acrylonitrile

### B44: (E/Z)-2-(5-((1-(2,6-dimethoxy-4-(1,4,5-trimethyl-6-oxo-1,6-dihydropyridin-3-yl)benzyl)piperidin-4-yl)oxy)-1-methyl-1,2,3,4-tetrahydroisoquinoline-2-carbonyl)-4,4-dimethylpent-2-enenitrile

### B64: (E/Z)-2-(5-((1-(2,6-dimethoxy-4-(1,4,5-trimethyl-6-oxo-1,6-dihydropyridin-3-yl)benzyl)piperidin-4-yl)oxy)-1-methyl-1,2,3,4-tetrahydroisoquinoline-2-carbonyl)-3-(5-(trifluoromethyl)pyridin-2-yl)acrylonitrile

### B22: (E/Z)-2-(5-((1-(2,6-dimethoxy-4-(1,4,5-trimethyl-6-oxo-1,6-dihydropyridin-3-yl)benzyl)piperidin-4-yl)oxy)-1-methyl-1,2,3,4-tetrahydroisoquinoline-2-carbonyl)-3-(6-(trifluoromethyl)pyridin-2-yl)acrylonitrile

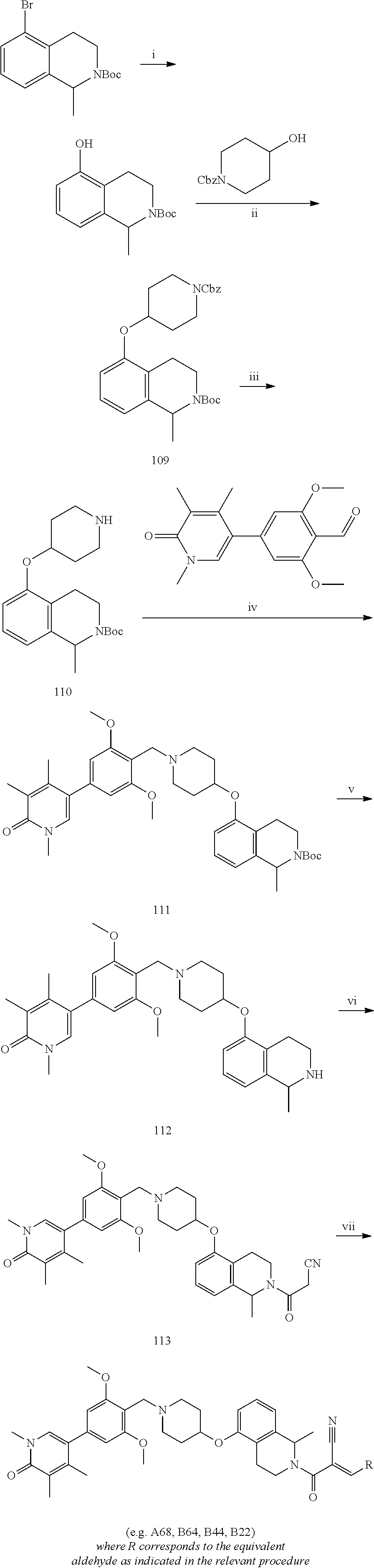

i) tBuXPhos-Pd-G3, KOH, 1,4-dioxane; ii) TPP, DIAD, DCM; iii) Pd/C, MeOH; iv) MP-CNBH₃, AcOH, MeOH; v) HCl (4 M in 1,4-dioxane); vi) 3-(3,5-Dimethyl-1H-pyrazol-1-yl)-3-oxopropanenitrile, DIPEA, 1,4-dioxane; vii) aldehyde (as indicated by structure in experimental), piperidine, THF.

109: To a stirred solution of tert-butyl 5-hydroxy-1-methyl-3,4-dihydroisoquinoline-2(1H)-carboxylate (prepared as described in WO2022/117678, 1.0 g, 3.80 mmol) and benzyl 4-hydroxypiperidine-1-carboxylate (1.1 g, 4.56 mmol) in DCM (20 mL) was added triphenylphosphine (1.49 g, 5.70 mmol), followed by DIAD (1.15 g, 5.70 mmol) at 0° C. The reaction mixture was stirred at RT for 12 h then was quenched with ice-cold water and extracted with DCM. The combined organic layers were concentrated in vacuo and purified by silica gel column chromatography (gradient=30-40% EtOAc in n-hexane) to obtain tert-butyl 5-((1-((benzyloxy)carbonyl)piperidin-4-yl)oxy)-1-methyl-3,4-dihydroisoquinoline-2(1H)-carboxylate (1.0 g, 2.08 mmol, 54.8% yield). LCMS m/z=380.1 [M+H-Boc]⁺.

110: To a stirred solution of tert-butyl 5-((1-((benzyloxy)carbonyl)piperidin-4-yl)oxy)-1-methyl-3,4-dihydroisoquinoline-2(1H)-carboxylate (3.5 g, 7.28 mmol) in MeOH (40 mL) was added Pd/C (0.77 g, 0.72 mmol) and the reaction mixture was purged with N2. The reaction mixture was stirred at RT for 16 h then was filtered through celite and concentrated in vacuo to afford tert-butyl 1-methyl-5-(piperidin-4-yloxy)-3,4-dihydroisoquinoline-2(1H)-carboxylate (3.0 g, 6.41 mmol, 88% yield). LCMS m/z=347.1 [M+H]⁺.

111: By general procedure 1 using MP-CNBH₃ (1 equiv.) and sodium acetate (1.5 equiv.) in DCE. Obtained 500 mg, 79% yield. LCMS m/z=632.4 [M+H]⁺.

112: By general procedure 2. Obtained 450 mg, 98% yield. LCMS m/z=532.1 [M+H]⁺.

113: By general procedure 3. Obtained 360 mg, 75% yield. LCMS m/z=599.3 [M+H]⁺.

| | | | | LCMS |
|---|---|---|---|---|
| Com- | | | | m/z |
| pound | Structure | Procedure | Yield | [M + H]⁺ |
| A68 | 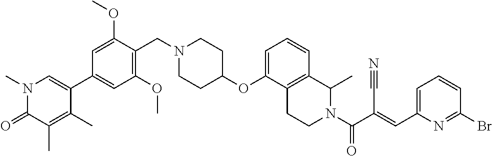 [SMILES: COc1cc(-c2cn(C)c(=O)c(C)c2C)cc(OC)c1CN1CCC(Oc2cccc3c2CCN(C(=O)/C(C#N)=C/c2cccc(Br)n2)C3C)CC1] | 4 | 55 mg, 28.6% | 766.2 |
| B64 | 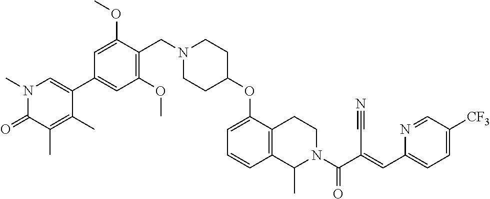 [SMILES: COc1cc(-c2cn(C)c(=O)c(C)c2C)cc(OC)c1CN1CCC(Oc2cccc3c2CCN(C(=O)/C(C#N)=C/c2ccc(C(F)(F)F)cn2)C3C)CC1] | 4a | 45 mg, 24% | 756.2 |
| B44 | 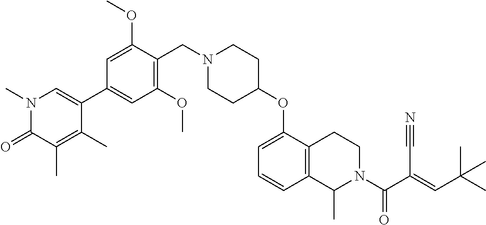 [SMILES: COc1cc(-c2cn(C)c(=O)c(C)c2C)cc(OC)c1CN1CCC(Oc2cccc3c2CCN(C(=O)/C(C#N)=C/C(C)(C)C)C3C)CC1] | 6 | 57 mg, 49% | 667.3 |
| B22 | 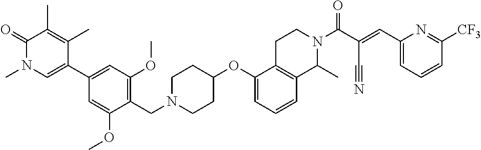 [SMILES: COc1cc(-c2cn(C)c(=O)c(C)c2C)cc(OC)c1CN1CCC(Oc2cccc3c2CCN(C(=O)/C(C#N)=C/c2cccc(C(F)(F)F)n2)C3C)CC1] | 4a | 15 mg, 7.8% | 756.3 |

### Compound A28: (E/Z)-2-(5-((1-(2,6-dimethoxy-4-(2-methyl-1-oxo-1,2-dihydro-2,7-naphthyridin-4-yl)benzyl)piperidin-4-yl)oxy)-1-methyl-1,2,3,4-tetrahydroisoquinoline-2-carbonyl)-4,4-dimethylpent-2-enenitrile

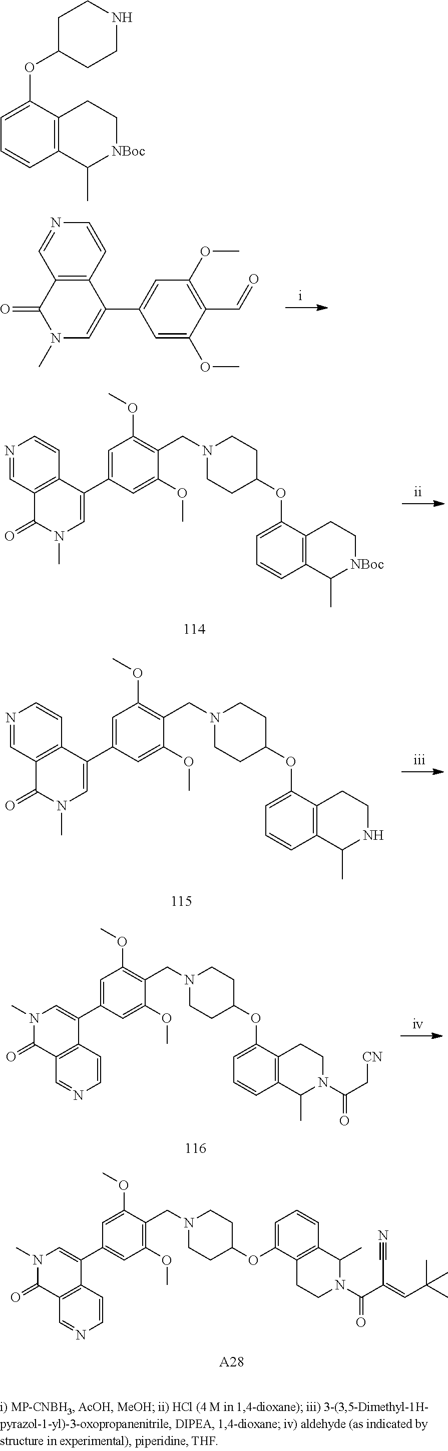

114: By general procedure 1 using MP-CNBH₃ (1 equiv.) in MeOH. Obtained 600 mg, 52.3% yield. LCMS m/z=655.4 [M+H]⁺.

115: By general procedure 2. Obtained 580 mg, 94% yield. LCMS m/z=592.1 [M+H]⁺

116: By general procedure 3. Obtained 500 mg, 58.7% yield. LCMS m/z=622.3 [M+H]⁺.

Compound A28: By general procedure 4. Obtained 7 mg, 3.1% yield. LCMS m/z=690.2 [M+H]⁺.

### Compound A62 and C28

### A62: 2-(6-(4-(2,6-dimethoxy-4-(1,4,5-trimethyl-6-oxo-1,6-dihydropyridin-3-yl) benzyl) piperazin-1-yl)-1-methyl-1,2,3,4-tetrahydroisoquinoline-2-carbonyl)-3-(1-fluorocyclopropyl)acrylonitrile

### C28: (E/Z)-3-(6-bromopyridin-2-yl)-2-(5-(4-(2,6-dimethoxy-4-(1,4,5-trimethyl-6-oxo-1,6-dihydropyridin-3-yl)benzyl)piperazin-1-yl)-1-methyl-1,2,3,4-tetrahydroisoquinoline-2-carbonyl)acrylonitrile

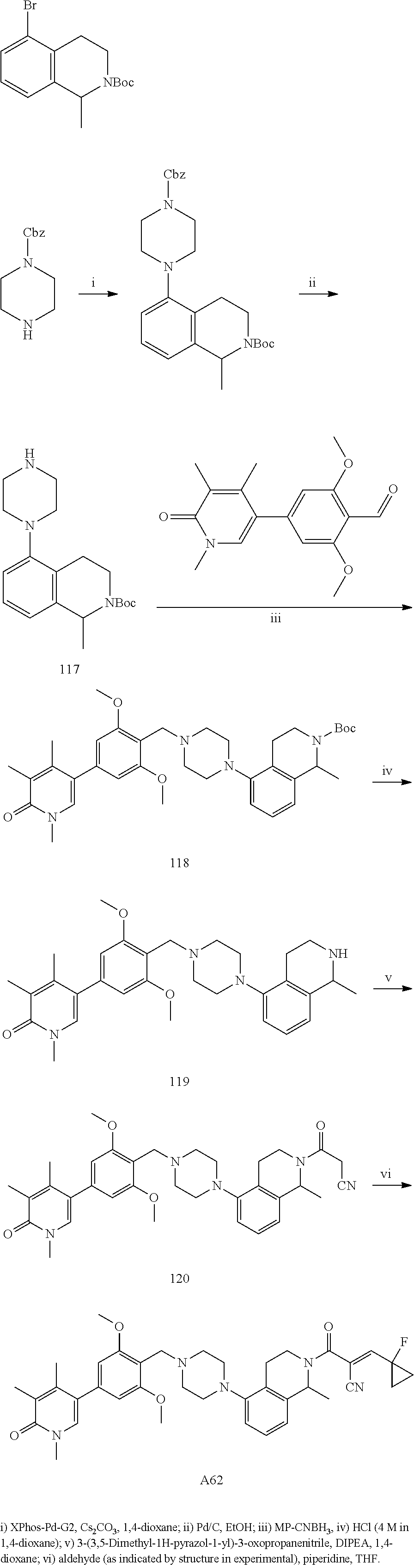

117: To a stirred solution of tert-butyl 5-(4-((benzyloxy) carbonyl) piperazin-1-yl)-1-methyl-3,4-dihydroisoquinoline-2 (1H)-carboxylate (3.0 g, 6.44 mmol) in EtOH (20 mL) was added Pd/C (2.0 g, 6.44 mmol) under N₂ atmosphere. The reaction was stirred at RT under H₂ atmosphere for 16 h then the reaction mixture was diluted with MeOH and filtered through celite. The filtrate was concentrated in vacuo to afford tert-butyl 1-methyl-5-(piperazin-1-yl)-3,4-dihydroisoquinoline-2 (1H)-carboxylate (2.1 g, 6.15 mmol, 95% yield). LCMS m/z=332.4 [M+H]⁺.

118: By general procedure 1 using MP-CNBH₃ (1 equiv.) in MeOH. Obtained 300 mg, 48.9% yield. LCMS m/z=617.7 [M+H]⁺.

119: By general procedure 2. Obtained 220 mg, 97% yield. LCMS m/z=517.7 [M+H]⁺.

120: By general procedure 3. Obtained 300 mg, 98.7% yield. LCMS m/z=584.6 [M+H]⁺.

| | | | Mass | LCMS |
|---|---|---|---|---|
| | | | isolated, | m/z |
| Name | Structure | Procedure | yield | [M + H]⁺ |
| A62 | 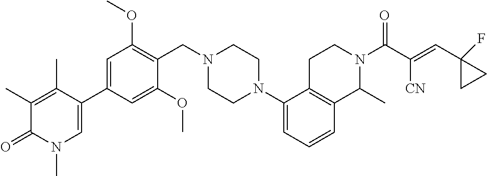 [SMILES: COc1cc(-c2cn(C)c(=O)c(C)c2C)cc(OC)c1CN1CCN(c2cccc3c2CCN(C(=O)/C(C#N)=C/C2(F)CC2)C3C)CC1] | 4 | 28 mg, 24.8% | 654.4 |
| C28 | 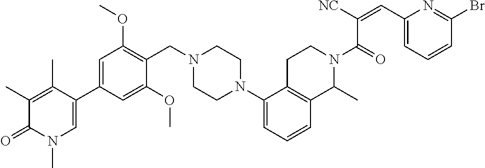 [SMILES: COc1cc(-c2cn(C)c(=O)c(C)c2C)cc(OC)c1CN1CCN(c2cccc3c2CCN(C(=O)/C(C#N)=C\c2cccc(Br)n2)C3C)CC1] | 4 | 15 mg, 11% yield | 751.1 |

### Compound A73, A63

### A73: 2-(2-(3-((4-(2,6-dimethoxy-4-(1,4,5-trimethyl-6-oxo-1,6-dihydropyridin-3-yl) benzyl) piperazin-1-yl) methyl)phenyl) pyrrolidine-1-carbonyl)-4,4-dimethylpentanenitrile

### A63: (E/Z)-3-(6-bromopyridin-2-yl)-2-(2-(3-((4-(2,6-dimethoxy-4-(1,4,5-trimethyl-6-oxo-1,6-dihydropyridin-3-yl) benzyl) piperazin-1-yl) methyl) phenyl) pyrrolidine-1-carbonyl) acrylonitrile

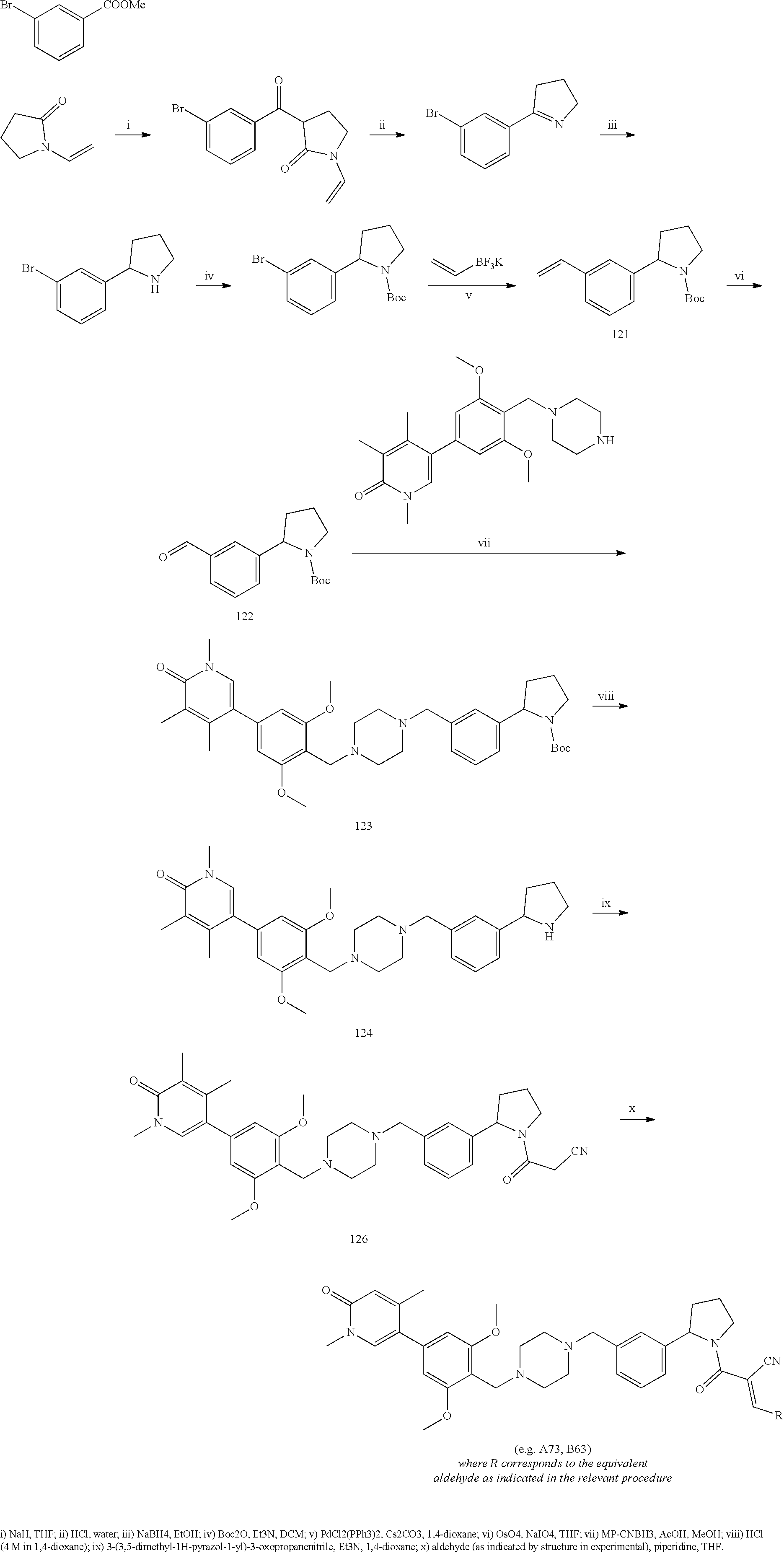

122: To a stirred solution of tert-butyl 2-(3-bromophenyl)pyrrolidine-1-carboxylate (prepared as in WO2016/28971, 1.0 g, 3.07 mmol) in 1,4-dioxane (10 mL) and water (1 mL) was added Cs₂CO₃ (1.0 g, 3.07 mmol), potassium vinyltrifluoroborate (1.2 g, 9.20 mmol) and the reaction vessel was purged with N₂ for 10 mins. PdCl₂(dppf)·DCM (0.25 g, 0.31 mmol) was added and the reaction mixture was stirred at 85° C. for 16 h. The reaction mixture was diluted with water and extracted with EtOAc. The organic layer was dried over anhydrous sodium sulphate and concentrated in vacuo. The resulting residue was purified by silica gel column chromatography (gradient=EtOAc in n-hexane) to afford tert-butyl 2-(3-vinylphenyl)pyrrolidine-1-carboxylate (2.1 g, 7.32 mmol, 75% yield). LCMS m/z=272.1 [M+H]⁺.

123: To a solution of tert-butyl 2-(3-vinylphenyl)pyrrolidine-1-carboxylate (2.0 g, 7.32 mmol) in water (1 mL) and 1,4-dioxane (20 mL) was added of sodium periodate (3.13 g, 14.6 mmol) and N-methylmorpholine (0.37 g, 3.66 mmol) at 0° C. OsO₄ (4.65 g, 0.73 mmol) was added slowly at 0° C. and the reaction mixture was stirred at RT for 2 h before being diluted with water and extracted with EtOAc. The organic layer was washed with brine solution, dried over anhydrous sodium sulphate, filtered, concentrated in vacuo and purified by silica gel column chromatography (gradient=20-26% EtOAc in n-hexane) to afford tert-butyl 2-(3-formylphenyl)pyrrolidine-1-carboxylate (1.8 g, 6.54 mmol, 89% yield). LCMS m/z=276.1 [M+H]⁺.

124: By general procedure 1 using MP-CNBH₃ (1 equiv.) in MeOH. Obtained 300 mg, 44.2% yield. LCMS m/z=631.1 [M+H]⁺.

125: By general procedure 2. Obtained 220 mg, 87% yield. LCMS m/z=531.1 [M+H]⁺.

126: By general procedure 3. Obtained 450 mg, 56.9% yield. LCMS m/z=598.3 [M+H]⁺.

| | | | Mass | LCMS |
|---|---|---|---|---|
| | | | isolated, | m/z |
| Name | Structure | Procedure | yield | [M + H]⁺ |
| A73 | 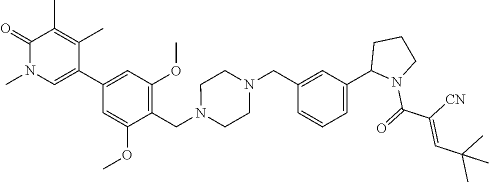 [SMILES: COc1cc(-c2cn(C)c(=O)c(C)c2C)cc(OC)c1CN1CCN(Cc2cccc(C3CCCN3C(=O)/C(C#N)=C/C(C)(C)C)c2)CC1] | 4 | 6 mg, 3.6% | 680.3 |
| A63 | 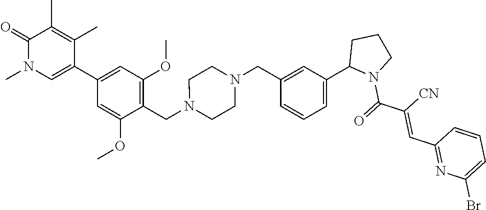 [SMILES: COc1cc(-c2cn(C)c(=O)c(C)c2C)cc(OC)c1CN1CCN(Cc2cccc(C3CCCN3C(=O)/C(C#N)=C/c3cccc(Br)n3)c2)CC1] | 4 | 4.9 mg, 2.7% | 767.6 |
| B37 | 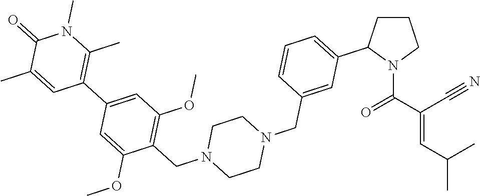 [SMILES: COc1cc(-c2cc(C)c(=O)n(C)c2C)cc(OC)c1CN1CCN(Cc2cccc(C3CCCN3C(=O)/C(C#N)=C/C(C)C)c2)CC1] | 4 | 39 mg, 19% | 652.3 |
| B26 | 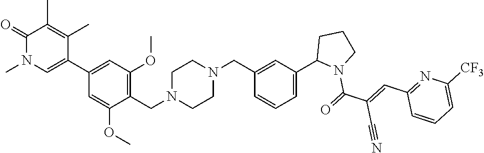 [SMILES: COc1cc(-c2cn(C)c(=O)c(C)c2C)cc(OC)c1CN1CCN(Cc2cccc(C3CCCN3C(=O)/C(C#N)=C/c3cccc(C(F)(F)F)n3)c2)CC1] | 4 | 17 mg, 8.9% | 755.2 |
| B56 | 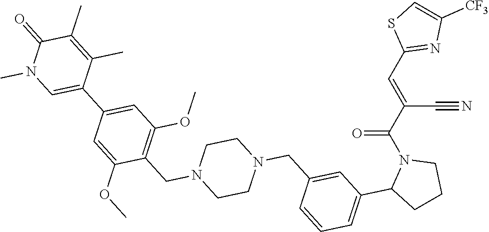 [SMILES: COc1cc(-c2cn(C)c(=O)c(C)c2C)cc(OC)c1CN1CCN(Cc2cccc(C3CCCN3C(=O)/C(C#N)=C/c3nc(C(F)(F)F)cs3)c2)CC1] | 4 | 10 mg, 5% | 761.2 |

### Compound A33: (E/Z)-2-(2-(3-((4-(2,6-dimethoxy-4-(2-methyl-1-oxo-1,2-dihydro-2,7-naphthyridin-4-yl)benzyl)piperazin-1-yl)methyl)phenyl)pyrrolidine-1-carbonyl)-3(thiazol-2-yl)acrylonitrile

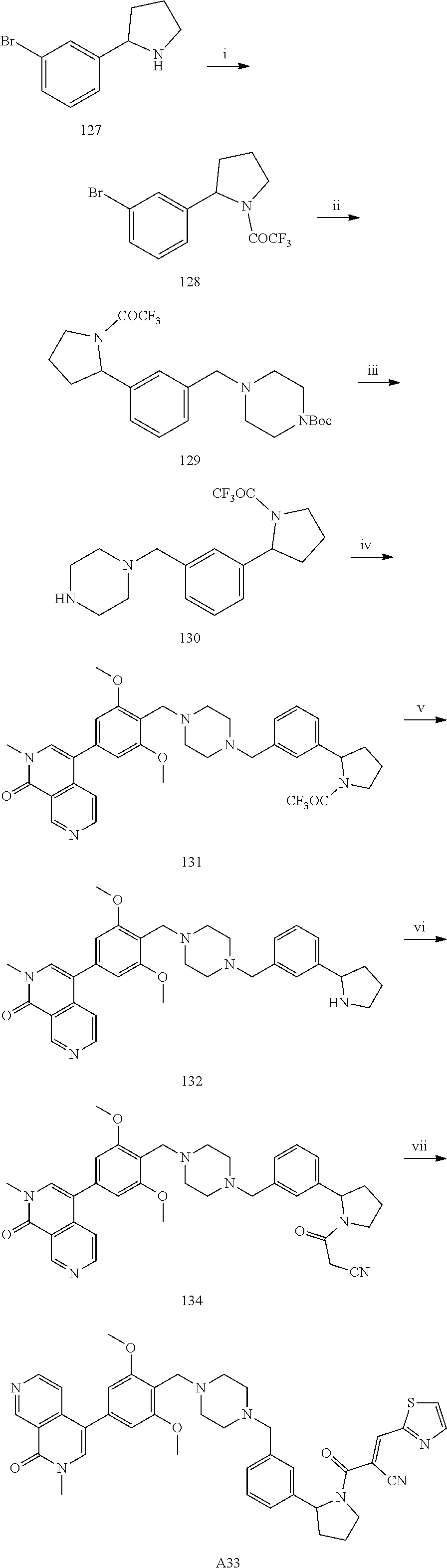

128: To a stirred solution of 2-(3-bromophenyl)pyrrolidine (1.4 g, 6.19 mmol) in DCM (30 mL) was added Et₃N (1.88 g, 18.5 mmol) and TFAA (1.56 g, 7.43 mmol) at 0° C. The reaction mixture was stirred at RT for 2 h then was diluted with water and extracted with DCM. The organic phase was concentrated in vacuo to afford 1-(2-(3-bromophenyl)pyrrolidin-1-yl)-2,2,2-trifluoroethan-1-one (1.5 g, 4.66 mmol, 88% yield). LCMS m/z=322.3 [M+H]⁺.

129: To a stirred solution of 1-(2-(3-bromophenyl)pyrrolidin-1-yl-2,2,2-trifluoroethan-1-one (1.5 g, 4.66 mmol) and potassium ((4-(tert-butoxycarbonyl)piperazin-1-yl)methy) trifluoroborate (1.4 g, 4.66 mmol) in THF (60 mL) and water (6 mL) was added Cs₂CO₃ (4.5 g, 13.9 mmol), followed by 2-dicyclohexylphosphino-2′,4′,6′-triisopropylbiphenyl (0.13 g, 0.27 mmol) and palladium(II) acetate (0.03 g, 0.14 mmol) under N2 atmosphere. The reaction mixture was stirred at 80° C. for 16 h. then was filtered through celite and concentrated in vacuo. The resulting residue was purified by silica gel column chromatography (35% EtOAc in n-hexane) to afford tert-butyl 4-(3-(1-(2,2,2-trifluoroacetyl)pyrrolidin-2-yl)benzyl)piperazine-1-carboxylate (1.1 g, 1.97 mmol, 42.3% yield). LCMS m/z=442.4 [M+H]⁺.

130: By general procedure 2. Obtained 480 mg, 86% yield. LCMS m/z=342.3 [M+H]⁺.

131: By general procedure 1 using MP-CNBH₃ (1 equiv.) in MeOH. Obtained 230 mg, 27.1% yield. LCMS m/z=650.2 [M+H]⁺.

132: By general procedure 20. the crude was suspended in ice-cold water and was extracted with EtOAc. The organic portion was washed with saturated aqueous brine solution and concentrated in vacuo. Obtained 110 mg, 74% yield). LCMS m/z=553.4 [M+H]⁺.

133: By general procedure 3. Obtained 100 mg, 50.3% yield. LCMS m/z=621.3 [M+H]⁺.

Compound A33: By general procedure 4 in EtOH. Obtained 60 mg, 56.4% yield. LCMS m/z=716.0 [M+H]⁺.

### Compound A38: (E/Z)-2-(2-(4-((4-(2,6-dimethoxy-4-(2-methyl-1-oxo-1,2-dihydro-2,7-naphthyridin-4-yl)benzyl)piperazin-1-yl)methyl)phenyl)pyrrolidine-1-carbonyl)-3-(thiazol-2-yl)acrylonitrile

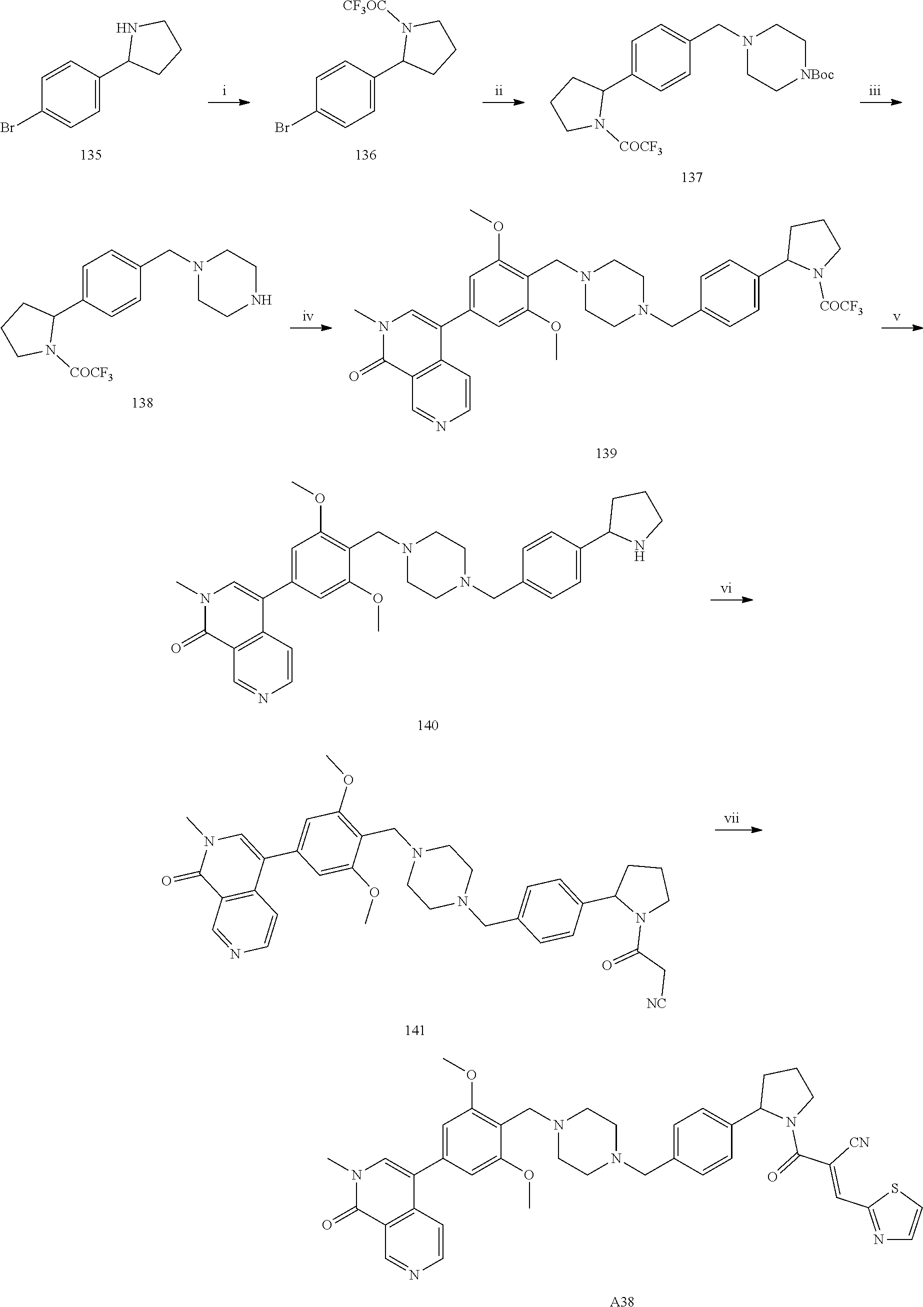

135: Prepared as described in US2010/35883. Obtained 2.0 g, 8.84 mmol.

136: To a stirred solution of 2-(4-bromophenyl)pyrrolidine (12.0 g, 8.84 mmol) and Et3N (13.4 mL, 26.5 mmol) in DCM (40 mL) at 0° C. was added trifluoroacetic anhydride (1.5 mL, 10.6 mmol) dropwise over 15 min. The reaction mixture was stirred at RT for 16 h then was diluted with water and extracted with DCM. The organic layer was dried over anhydrous Na₂SO₄ and concentrated in vacuo. The resulting residue was purified by silica gel column chromatography (gradient=7% EtOAc in n-hexane) to afford 1-(2-(4-bromophenyl)pyrrolidin-1-yl)-2,2,2-trifluoroethan-1-one (1.8 g, 5.31 mmol, 60% yield). LCMS m/z=322.2 [M+H]⁺.

137: To a stirred solution of 1-(2-(4-bromophenyl)pyrrolidin-1-yl)-2,2,2-trifluoroethan-1-one (1.5 g, 4.66 mmol) and potassium ((4-(tert-butoxycarbonyl)piperazin-1-yl)methyl)trifluoroborate (1.4 g, 4.66 mmol) in THF (60 mL) and water (6 mL) was added Cs₂CO₃(4.5 g, 13.9 mmol). 2-Dicyclohexylphosphino-2′,4′,6′-triisopropylbiphenyl (0.133 g, 0.28 mmol) and palladium(II) acetate (0.031 g, 0.14 mmol) were added under N2 atmosphere and the reaction mixture was stirred at 80° C. for 16 h. The reaction mixture was filtered through celite and the filtrate was concentrated in vacuo and purified by silica gel column chromatography (gradient=35% EtOAc in hexane) to afford tert-butyl 4-(4-(1-(2,2,2-trifluoracetyl)pyrrolidin-2-yl)benzyl)piperazine-1-carboxylate (0.9 g, 1.99 mmol, 42.9% yield). LCMS m/z=442.0 [M+H]⁺.

138: By general procedure 2. Obtained 500 mg, 96% yield. LCMS m/z=342.3 [M+H]⁺.

138: By general procedure 1 using MP-CNBH₃. Obtained 380 mg, 42.7% yield. LCMS m/z=650.0 [M+H]⁺.

139: By general procedure 20. The crude was diluted with 15% MeOH in DCM and was washed with water, brine solution and dried over anhydrous sodium sulphate before being concentrated in vacuo. Obtained 120 mg, 30% yield. LCMS m/z=554.1 [M+H]°.

140: By general procedure 3. Obtained 120 mg, 80% yield. LCMS m/z=622.0 [M+H]⁺.

Compound A38: By general procedure 4. Obtained 20 mg, 16.1% yield. LCMS m/z=716.2 [M+H]⁺.

### Compound A34: (E/Z)-2-(2-(4-(4-(2,6-dimethoxy-4-(2-methyl-1-oxo-1,2-dihydro-2,7-naphthyridin-4-yl)benzyl)piperazine-1-carbonyl)phenyl)pyrrolidine-1-carbonyl)-3-(thiazol-2-yl)acrylonitrile

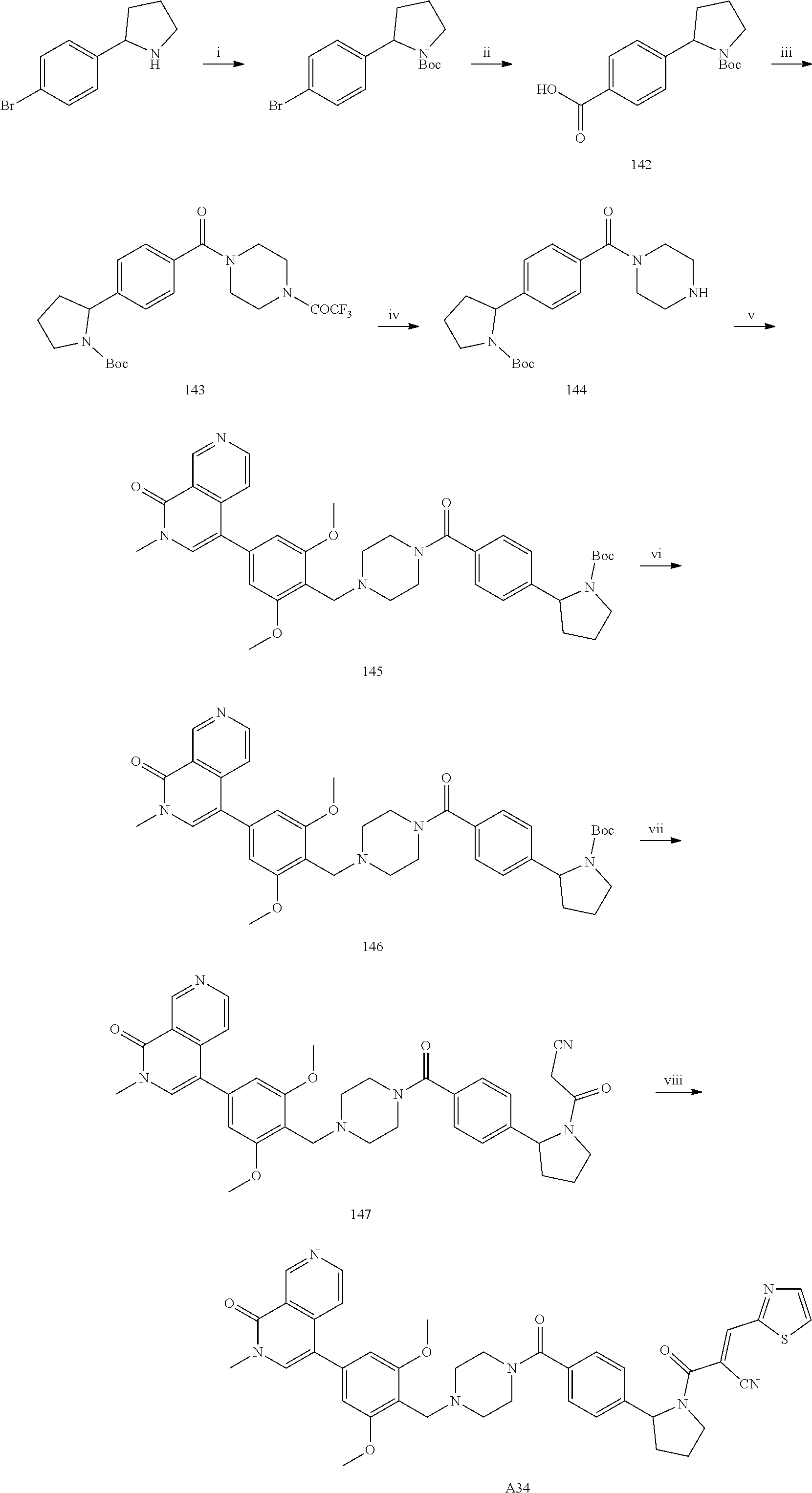

141: To a stirred solution of 2-(4-bromophenyl)pyrrolidine (12.1 g, 53.1 mmol) in DCM (200 mL) and was added Et₃N (22 mL, 159 mmol) and di-tert-butyl dicarbonate (14.7 mL, 64.1 mmol) at 0° C. The reaction mixture was stirred at RT for 16 h then was quenched with water and extracted with DCM. The combined organic extracts were washed with brine, dried over anhydrous sodium sulphate and concentrated in vacuo. The resulting residue was purified by silica gel column chromatography (gradient=0-15% EtOAc in n-hexane) to afford tert-butyl 2-(4-bromophenyl)pyrrolidine-1-carboxylate (15 g, 78% yield. LCMS m/z=325.1 [M+H]⁺.

142: To a stirred solution of sodium formate (0.64 g, 9.20 mmol) in DMF (15 mL) was added DIPEA (1.1 mL, 6.13 mmol) and Ac₂O (0.6 mL, 6.13 mmol). The reaction mixture was stirred at RT for 1 h under N₂ atmosphere before being purged with N2. Tert-butyl 2-(4-bromophenyl)pyrrolidine-1-carboxylate (1.0 g, 3.07 mmol), Pd(OAc)₂ (0.069 g, 0.307 mmol) and 1,1′-bis(diphenylphosphino)ferrocene (0.17 g, 0.31 mmol) were added to the reaction mixture which was stirred at 120° C. for 16 h under N₂ atmosphere. The reaction mixture was partitioned between water and EtOAc and the mixture was acidified to pH 1 with HCl (2 M). The aqueous layer was extracted with EtOAc, washed with brine solution, dried over Na₂SO₄ and concentrated in vacuo. The crude was purified silica gel column chromatography (gradient=0-60% EtOAc in n-hexane) to afford 4-(1-(tert-butoxycarbonyl)pyrrolidin-2-yl)benzoic acid (710 mg, 2.29 mmol, 74.7% yield). LCMS m/z=290.0 [M+H]⁺.

143: By general procedure 6. Obtained 400 mg, 56.9% yield. LCMS m/z=478.1 [M+H]⁺.

144: By general procedure 20. The crude was diluted with water and extracted with DCM. The organic layer was concentrated in vacuo. Obtained 280 mg, 89% yield. LCMS m/z=382.2 [M+H]⁺.

145: By general procedure 1 using MP-CNBH₃. Obtained 230 mg, 33.3% yield. LCMS m/z=668.3 [M+H]⁺.

146: By general procedure 2. Obtained 230 mg, 99.3% yield. LCMS m/z=588.1 [M+H]⁺.

147: By general procedure 3. Obtained 130 mg, 35% yield. LCMS m/z=634.2 [M+H]⁺.

Compound A34: By general procedure 4. Obtained 23 mg, 19.2% yield. LCMS m/z=730.2 [M+H]⁺.

### Compound A76: (E/Z)—N-(5-(3-(1-(2-cyano-3-(thiazol-2-yl)acryloyl)pyrrolidin-2-yl)phenoxy)pentyl)-7-(4-((dimethylamino)methyl)-3,5-dimethoxyphenyl)-5-methyl-4-oxo-4,5-dihydrothieno[3,2-c]pyridine-2-carboxamide

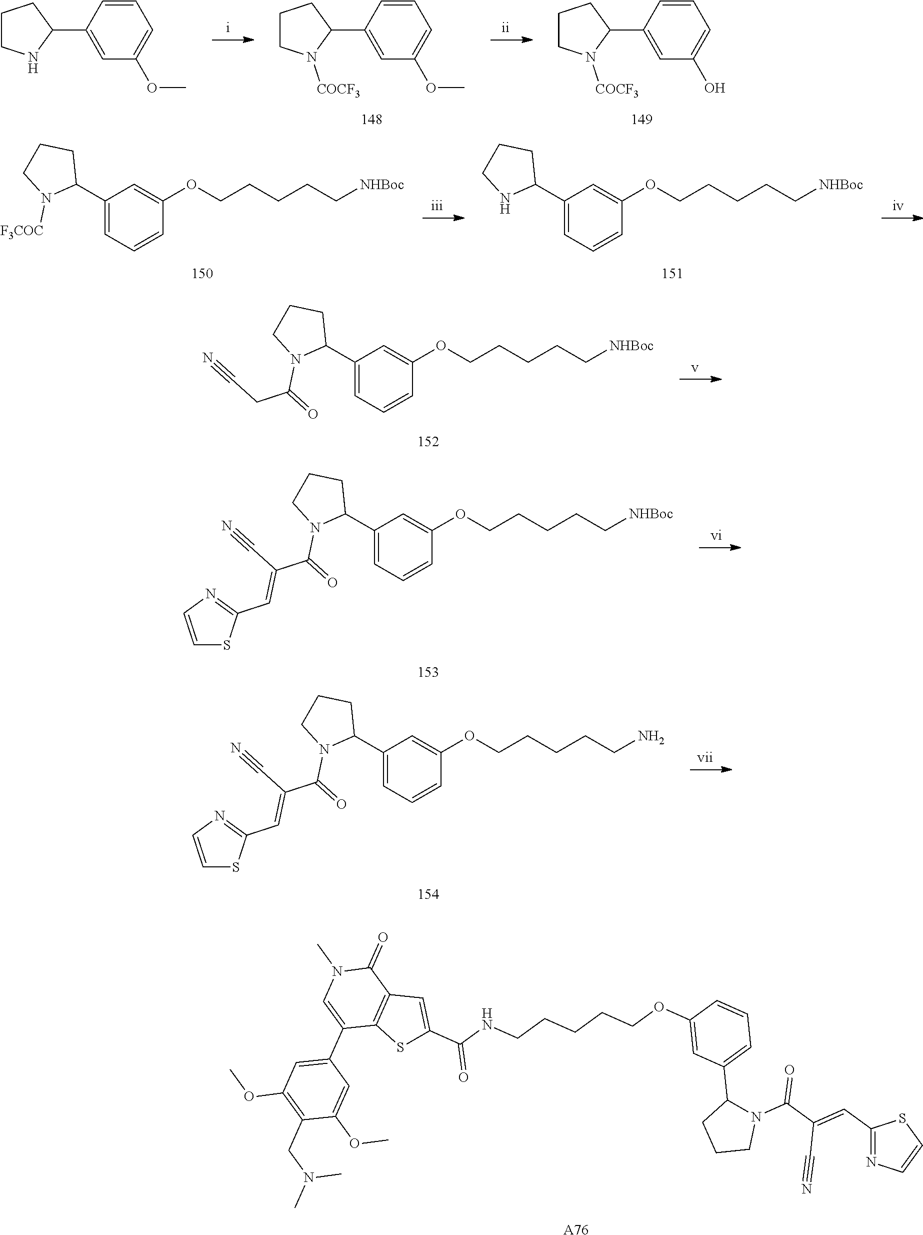

148: To a stirred solution of 2-(3-methoxyphenyl)pyrrolidine (4.0 g, 22.56 mmol) in DCM (40 mL) was added Et₃N (9.49 mL, 67.68 mmol) and TFAA (4.7 mL, 33.84 mmol) at 0° C. under N₂ atmosphere. The reaction mixture was stirred at RT for 8 h before being quenched with cold water and extracted with DCM. The combined organic layers were dried over anhydrous Na₂SO₄, filtered and concentrated in vacuo. The resulting crude residue was purified by silica gel column chromatography (gradient=5% EtOAc in heptane) to afford 2,2,2-trifluoro-1-[2-(3-methoxyphenyl)pyrrolidin-1-yl]ethanone (4.5 g, 72% yield). LCMS m/z=274.1 [M+H]⁺.

149: To a solution of 2,2,2-trifluoro-1-[2-(3-methoxyphenyl)pyrrolidin-1-yl]ethanone (4.5 g, 16.47 mmol) in DCM (40 mL) was added BBr₃ (1 M in DCM, 24.7 mL, 24.7 mmol) dropwise at 0° C. The reaction mixture was stirred at 0° C. for 3 h before being quenched with MeOH at 0° C. The reaction mixture was diluted with ice water and extracted with DCM. The combined organic layers were dried over anhydrous Na₂SO₄, filtered and concentrated in vacuo to afford 2,2,2-trifluoro-1-[2-(3-hydroxyphenyl)pyrrolidin-1-yl]ethanone (4 g, 95% yield). LCMS m/z=260.1 [M+H]⁺.

150: To a stirred solution of 2,2,2-trifluoro-1-[2-(3-hydroxyphenyl)pyrrolidin-1-yl]ethanone (4.0 g, 15.44 mmol) in toluene (40 mL) was added tert-butyl N-(5-hydroxypentyl)carbamate (3.76 g, 18.53 mmol) and cyanomethyl tributyl phosphorane (5.58 g, 23.16 mmol) at RT. The reaction mixture was heated at 80° C. for 16 h before being concentrated in vacuo. The resulting crude was diluted with water and extracted with EtOAc. The combined organic layers were dried over anhydrous Na₂SO₄, filtered and concentrated in vacuo. The crude material was purified by silica gel column chromatography (gradient=25% EtOAc in heptane) to afford tert-butyl N-[5-[3-[1-(2,2,2-trifluoroacetyl)pyrrolidin-2-yl]phenoxy]pentyl]carbamate (4.50 g, 65% yield). LCMS m/z=445.3 [M+H]⁺.

151: Followed general procedure 20. Obtained 3.5 g crude. LCMS m/z=349.3 [M+H]⁺

152: By general procedure 3. Obtained 2.0 g, 84% yield. LCMS m/z=416.3 [M+H]⁺.

153: By general procedure 4. Obtained 1.5 g, 61% yield. LCMS m/z=509.3 [M−H]⁺

154: By general procedure 2. After silica gel column chromatography (gradient=5% MeOH in DCM), obtained 170 mg, 42% yield. LCMS m/z=411.2 [M+H]⁺.

Compound A76: By general procedure 6 using acid prepared according to WO2021155100. After prep-HPLC, obtained 2 mg, 10% yield. LCMS m/z=795.4 [M+H]⁺.

### Compound A74: (E/Z)—N-(2-((2-(2-cyano-3-(thiazol-2-yl)acryloyl)-1-methyl-1,2,3,4-tetrahydroisoquinolin-8-yl)oxy)ethyl)-7-(4-((dimethylamino)methyl)-3,5-dimethoxyphenyl)-5-methyl-4-oxo-4,5-dihydrothieno[3,2-c]pyridine-2-carboxamide

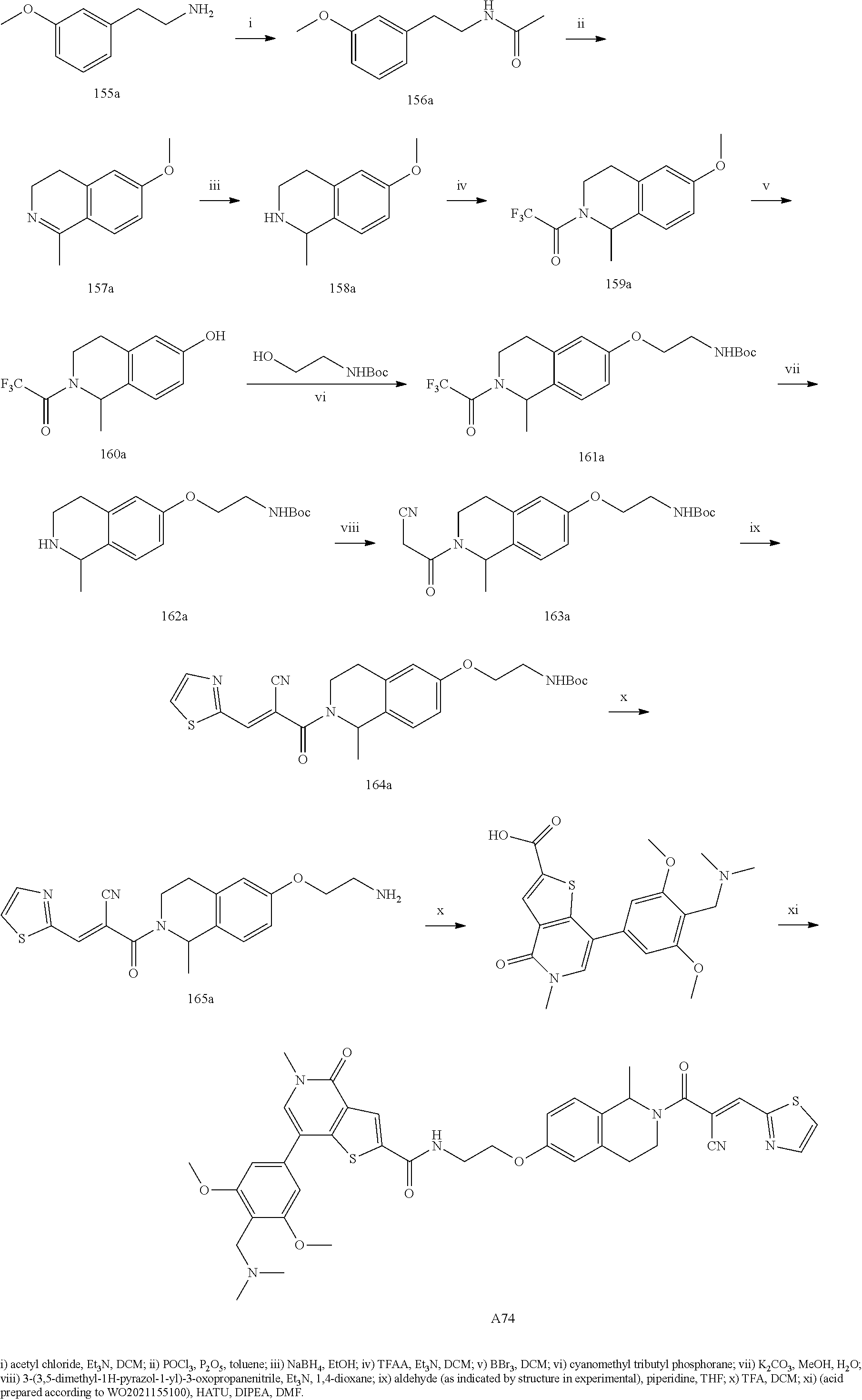

156a: To a stirred solution of 2-(3-methoxyphenyl)ethanamine (25 g, 165.3 mmol) in DCM (200 mL) was added Et3N (35.8 mL, 247.99 mmol) and acetyl chloride (13.0 mL, 181.87 mmol) dropwise at 0° C. under N₂ atmosphere. The reaction mixture was stirred at RT for 12 h. before being quenched with cold water and extracted with DCM. The combined organic layers were washed with brine, dried over anhydrous Na₂SO₄, filtered and concentrated in vacuo to afford 6-methoxy-1-methyl-3,4-dihydroisoquinoline (18 g, 56% yield). LCMS m/z=194.5 [M+H]⁺

157a: To a stirred solution of N-[2-(3-methoxyphenyl)ethyl]acetamide (10 g, 51.7 mmol) in toluene (100 mL) was added POCs (16.92 mL, 181.1 mmol) and P₂O₅ (18.30 g, 129.3 mmol) under N₂ atmosphere. The reaction mixture was refluxed for 12 h before being cooled to 0° C., basified with NaOH (25% aqueous solution) and extracted with EtOAc. The combined organic layers were dried over anhydrous Na₂SO₄, filtered and concentrated in vacuo. The crude material was purified by silica gel column chromatography (gradient=10% EtOAc in heptane) to afford 6-methoxy-1-methyl-3,4-dihydroisoquinoline (8 g, 88% yield). LCMS m/z=176.1 [M+H]⁺

158a: To a stirred solution of 6-methoxy-1-methyl-3,4-dihydroisoquinoline (8 g, 45.7 mmol) in EtOH (100 mL) was added NaBH₄ (2.59 g, 68.46 mmol) portion-wise at 0° C. under N₂ atmosphere. The reaction mixture was warmed to RT and stirred for 12 h before being concentrated in vacuo. The crude residue was diluted with water and extracted with EtOAc. The combined organic layers were dried over anhydrous Na₂SO₄, filtered and concentrated in vacuo to afford 6-methoxy-1-methyl-1,2,3,4-tetrahydroisoquinoline (5 g, 62% yield). LCMS m/z=178.0 [M+H]⁺

159a: To a stirred solution of 6-methoxy-1-methyl-1,2,3,4-tetrahydroisoquinoline (8.0 g, 45.13 mmol) in DCM (80 mL) was added Et₃N (13.0 g, 90.27 mmol) and TFAA (7.54 mL, 54.16 mmol) at 0° C. under N2 atmosphere. The reaction mixture was warmed to RT and stirred for 2 h before being diluted with ice water and extracted with DCM. The combined organic layers were dried over anhydrous Na₂SO₄, filtered and concentrated in vacuo. The crude material was purified by silica gel column chromatography (gradient=5% EtOAc in heptane) to afford 2,2,2-trifluoro-1-(6-methoxy-1-methyl-3,4-dihydro-1H-isoquinolin-2-yl)ethanone (7 g, 57% yield). LCMS m/z=274.2 [M+H]⁺

160a: To a stirring solution of 2,2,2-trifluoro-1-(6-methoxy-1-methyl-3,4-dihydro-1H-isoquinolin-2-yl)ethanone (4.0 g, 14.63 mmol) in DCM (50 mL) was added BBr₃ (1 M in DCM, 21.9 mL, 21.9 mmol) at 0° C. under N₂ atmosphere. The reaction mixture was stirred at RT for 2 h. before being quenched with MeOH and concentrated in vacuo. The resulting crude residue was diluted with ice water and extracted with EtOAc. The combined organic layers were dried over anhydrous Na₂SO₄, filtered and concentrated in vacuo to afford 2,2,2-trifluoro-1-(8-hydroxy-1-methyl-3,4-dihydro-1H-isoquinolin-2-yl)ethanone (3.5 g, 94% yield). LCMS m/z=260.1 [M+H]⁺

161a: To a stirring solution of 2,2,2-trifluoro-1-(8-hydroxy-1-methyl-3,4-dihydro-1H-isoquinolin-2-yl)ethanone (3.5 g, 13.50 mmol) in toluene (50 mL) was added tert-butyl N-(2-hydroxyethyl)carbamate (2.61 g, 16.2 mmol) and cyanomethyl tributyl phosphorane (4.88 g, 20.25 mmol) at RT. The reaction mixture was stirred at 80° C. for 16 h before being concentrated in vacuo. The resulting crude residue was diluted with ice-water and extracted with DCM. The combined organic layers were dried over anhydrous Na₂SO₄, filtered and concentrated in vacuo to afford tert-butyl N-[2-[[1-methyl-2-(2,2,2-trifluoroacetyl)-3,4-dihydro-1H-isoquinolin-6-yl]oxy]ethyl]carbamate (4.0 g crude) as a white solid. The crude product was used in the next step without further purification. LCMS m/z=805.4 [2M+H]⁺

162a: To a stirred solution of tert-butyl N-[2-[[1-methyl-2-(2,2,2-trifluoroacetyl)-3,4-dihydro-1H-isoquinolin-6-yl]oxy]ethyl]carbamate (4.0 g, 9.94 mmol) in MeOH (40 mL) and water (40 mL) was added K₂CO₃ (4.12 g, 29.82 mmol) at RT. The reaction mixture was stirred at 80° C. for 16 h before being concentrated in vacuo. The resulting crude residue was diluted with water and extracted with EtOAc. The combined organic layers were dried over anhydrous Na₂SO₄, filtered and concentrated in vacuo to afford tert-butyl N-[2-[(1-methyl-1,2,3,4-tetrahydroisoquinolin-6-yl)oxy]ethyl]carbamate (4.0 g crude). The crude product was used in the next step without further purification. LCMS m/z=307.2 [M+H]⁺

163a: Prepared following general procedure 3. Obtained 3.0 g, 61.6% yield. LCMS m/z=274.2 [M-Boc+H]⁺.

164a: Prepared following general procedure 4. Obtained 1.5 g, 60% yield. LCMS m/z=469.3 [M+H]⁺

165a: Prepared following general procedure 2. After silica gel column chromatography (gradient=10% MeOH in DCM), obtained 340 mg, 87% yield. LCMS m/z=369.3 [M+H]⁺.

Compound A74: By general procedure 6 using (E/Z)-2-(6-(2-aminoethoxy)-1-methyl-1,2,3,4-tetrahydroisoquinoline-2-carbonyl)-3-(thiazol-2-yl)acrylonitrile (Intermediate W-6) and acid prepared according to WO2021155100. After prep-HPLC, obtained 3 mg, 12% yield. LCMS m/z=754.2 [M+H]⁺.

### Compound A75: (E/Z)—N-(5-((2-(2-cyano-3-(thiazol-2-yl)acryloyl)-1-methyl-1,2,3,4-tetrahydroisoquinolin-6-yl)oxy)pentyl)-7-(4-((dimethylamino)methyl)-3,5-dimethoxy phenyl)-5-methyl-4-oxo-4,5-dihydrothieno[3,2-c]pyridine-2-carboxamide

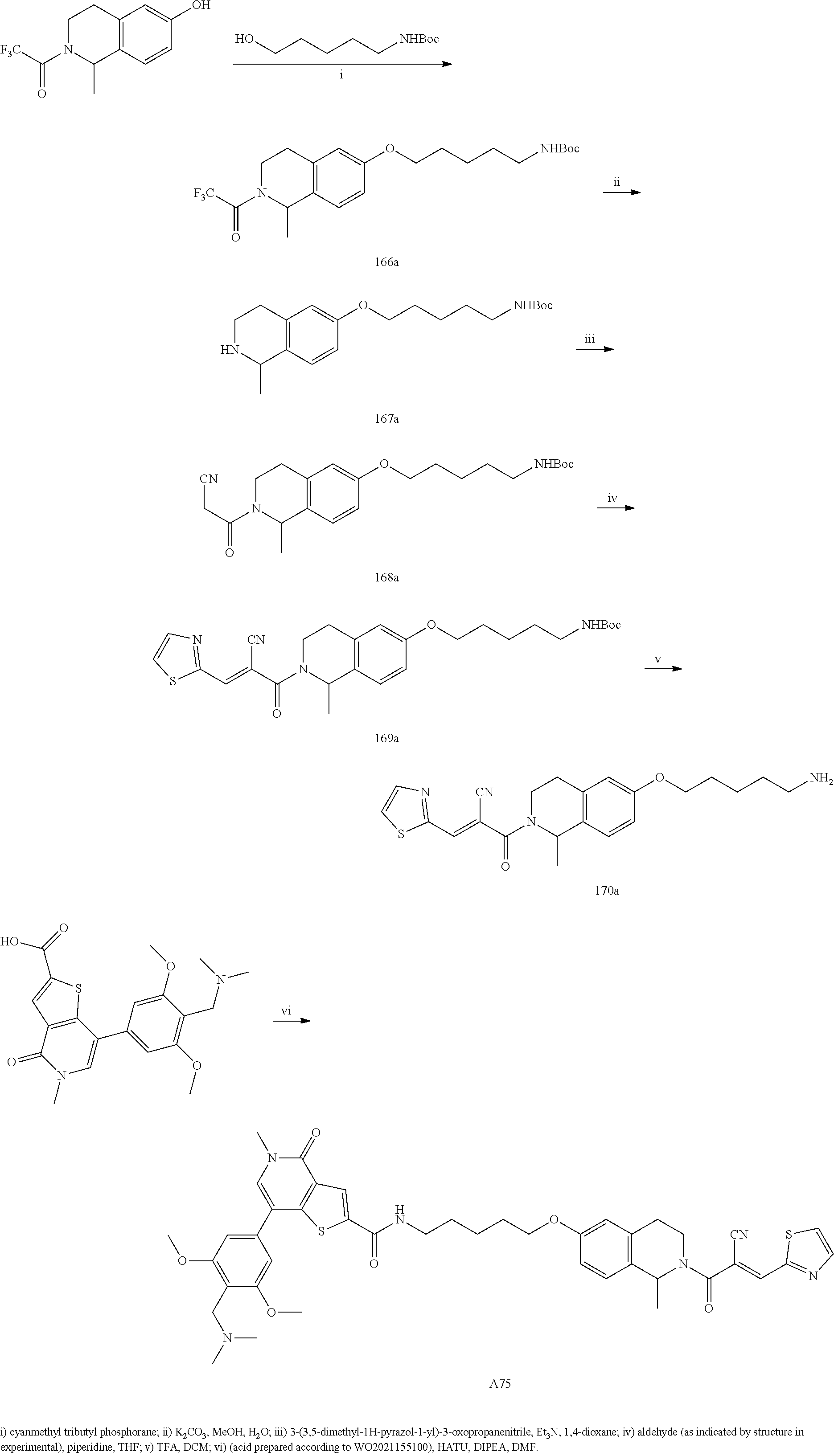

166a: To a stirring solution of 2,2,2-trifluoro-1-(6-hydroxy-1-methyl-3,4-dihydro-1H-isoquinolin-2-yl)ethanone (4.0 g, 15.43 mmol) in toluene (50 mL) was added tert-butyl N-(5-hydroxypentyl)carbamate (3.76 g, 18.51 mmol) and cyanomethyl tributyl phosphorane (5.57 g, 23.13 mmol) at RT. The reaction mixture was stirred at 80° C. for 16 h before being concentrated in vacuo. The resulting crude residue was diluted with water and extracted with DCM. The combined organic extracts were dried over anhydrous Na₂SO₄, filtered and concentrated in vacuo. The resulting crude material was purified by silica gel column chromatography (gradient=20% EtOAc in heptane) to afford tert-butyl N-[5-[[1-methyl-2-(2,2,2-trifluoroacetyl)-3,4-dihydro-1H-isoquinolin-8-yl]oxy]pentyl]carbamate (4.5 g, 65.7% yield). LCMS m/z=334.2 [M-Boc+H]⁺.

167a: To a stirring solution of tert-butyl N-[5-[[1-methyl-2-(2,2,2-trifluoroacetyl)-3,4-dihydro-1H-isoquinolin-6-yl]oxy]pentyl]carbamate (4.0 g, 9.0 mmol) in MeOH (10 mL) and water (10 mL) was added K₂CO₃ (3.73 g, 27.01 mmol) at RT. The reaction mixture was heated at 80° C. for 16 h before being quenched with water and extracted with EtOAc. The combined organic layers was dried over anhydrous Na₂SO₄, filtered and concentrated in vacuo. The crude material was purified by silica gel column chromatography (gradient=10% MeOH in DCM) to afford tert-butyl N-[5-[(1-methyl-1,2,3,4-tetrahydroisoquinolin-6 yl)oxy]pentyl]carbamate (3.0 g, 96% yield). LCMS m/z=349.4 [M+H]⁺.

168a: Prepared following general procedure 3. Obtained 2.0 g, 48% yield. LCMS m/z=316.3 [M−Boc+H]⁺.

169a: Prepared following general procedure 4. Obtained 2.2 g, 59% yield. LCMS m/z=411.3 [M+H]⁺

170a: Prepared following general procedure 2. After silica gel column chromatography (gradient=10% MeOH in DCM), obtained 400 mg, 98% yield. LCMS m/z=411.2 [M+H]⁺.

Compound A75: By general procedure 8 using (E/Z)-2-(6-((5-aminopentyl)oxy)-1-methyl-1,2,3,4-tetrahydroisoquinoline-2-carbonyl)-3-(thiazol-2-yl)acrylonitrile (Intermediate W-7) and acid prepared according to WO2021155100. After prep-HPLC, obtained 3.1 mg, 15% yield. LCMS m/z=795.8 [M+H]⁺.

### Further Examples of BRD9 Degraders

Overviews of various exemplary synthetic methods and general procedures that may be used to provide the compounds of the present disclosure are shown below.

### Compound B17: (E/Z)-2(3-(6-bromopyridin-2-yl)-2-cyanoacryloyl)-N-(2,6-dimethoxy-4-(1,4,5-trimethyl-6-oxo-1,6-dihydropyridin-3-yl)benzyl)-N,1-dimethyl-1,2,3,4-tetrahydroisoquinoline-6-carboxamide

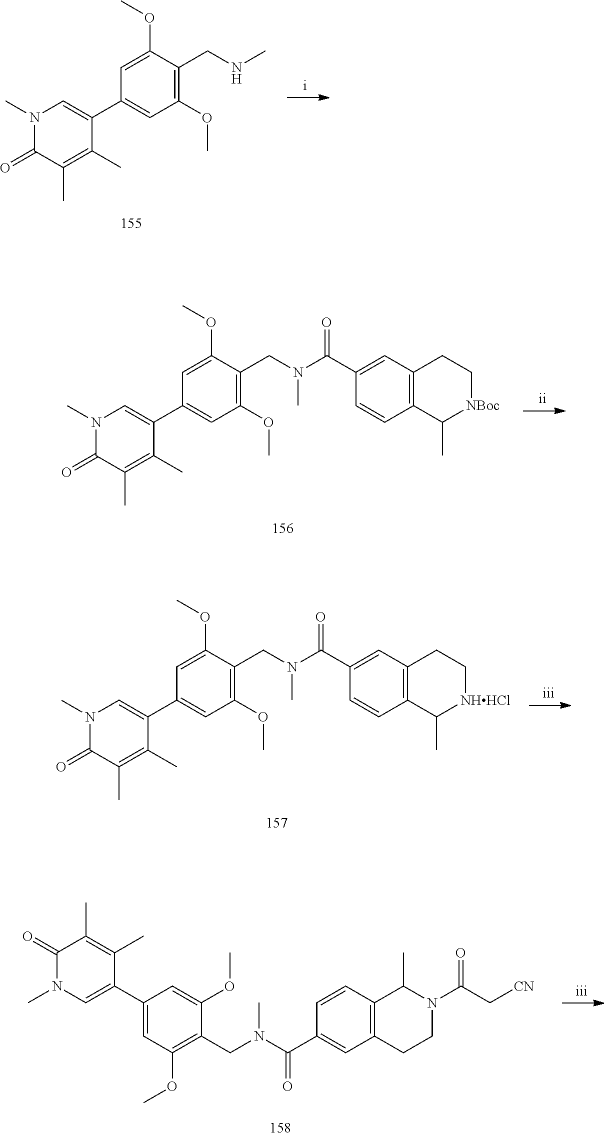

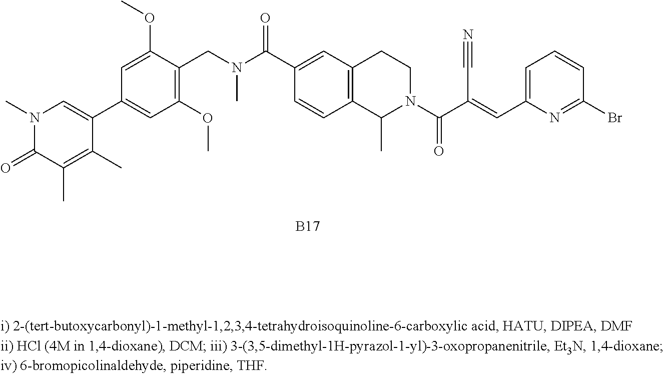

156: By general procedure 6. Obtained 800 mg, 48% yield. LCMS m/z=590.3 [M+H]⁺.

157: By general procedure 2. Obtained 260 mg, 64% yield. LCMS m/z=490.1 [M+H]⁺.

158: By general procedure 3. Obtained 120 mg, 40% yield. LCMS m/z=557.2[M+H]⁺.

Compound B17: By general procedure 4. Obtained 45 mg, 30% yield. LCMS m/z=726.2 [M+H]⁺.

### (E/Z)-2-(3-(6-bromopyridin-2-yl)-2-cyanoacryloyl)-N-(2,6-dimethoxy-4-(1,4,5-trimethyl-6-oxo-1,6-dihydropyridin-3-yl)benzyl)-N,1-dimethyl-1,2,3,4-tetrahydroisoquinoline-5-carboxamide (B16)

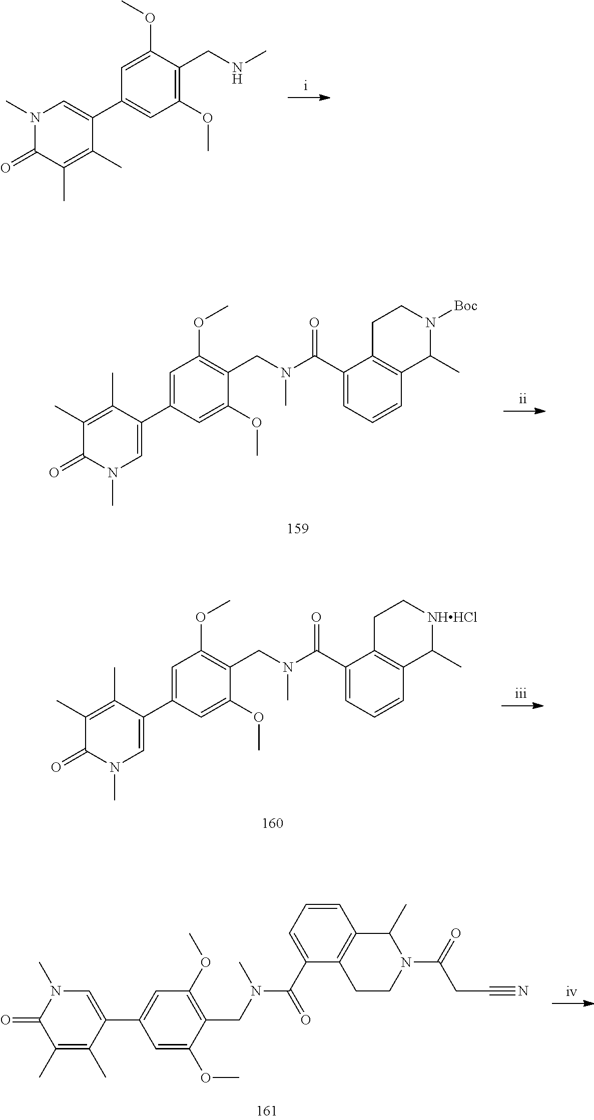

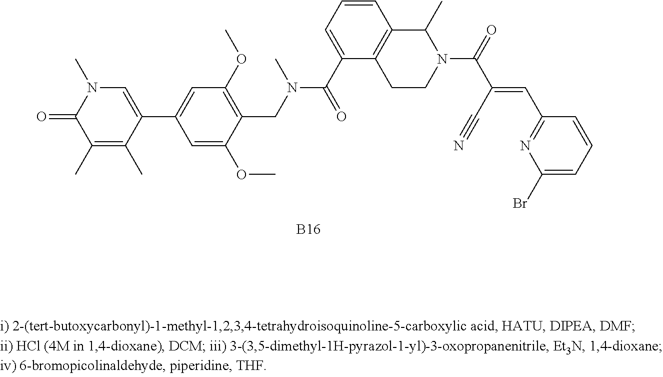

159: By general procedure 6. Obtained 299 mg, 42% yield. LCMS m/z=590.3 [M+H]⁺.

160: By general procedure 2. Obtained 280 mg, 99% yield. LCMS m/z=490.2 [M+H]⁺.

161: By general procedure 3. Obtained 210 mg, 72% yield. LCMS m/z=557.2 [M+H]⁺.

Compound B16: By general procedure 4. Obtained 84 mg. LCMS m/z=726.2 [M+H]⁺.

### (E/Z)-2-(5-((4-(2,6-dimethoxy-4-(1,4,5-trimethyl-8-oxo-1,6-dihydropyridin-3-yl)benzyl)-2-oxopiperazin-1-yl)methyl)-1-methyl-1,2,3,4-tetrahydroisoquinoline-2-carbonyl)-4,4-dimethylpent-2-enenitrile (B155)

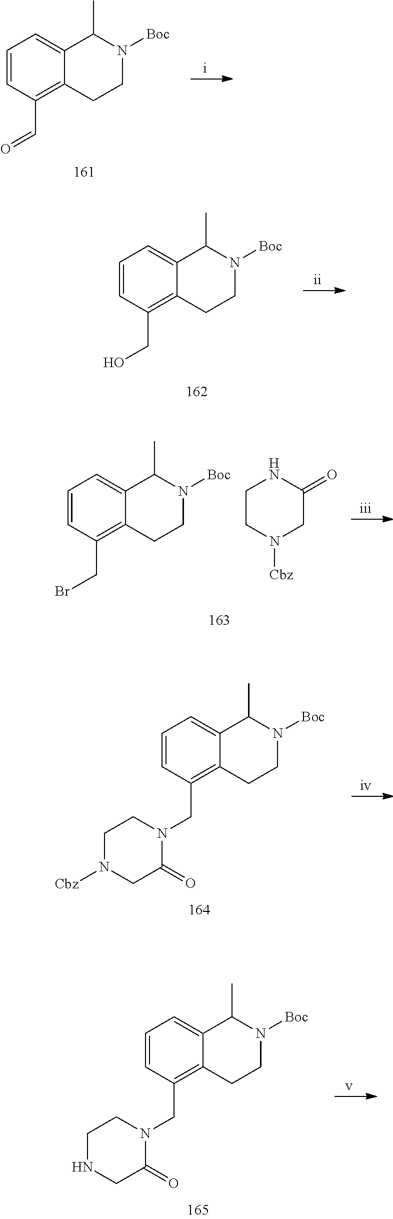

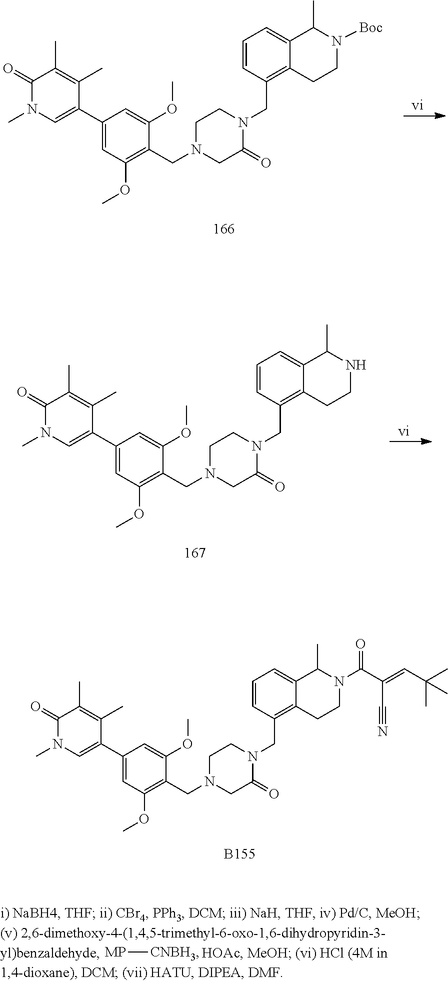

162: To a stirred solution of tert-butyl 5-formyl-1-methyl-3,4-dihydroisoquinoline-2(1H)-carboxylate (0.5 g, 1.82 mmol, 1.0 Eq) in THF (5 ml) was added NaBH₄ (0.10 g, 2.72 mmol, 1.5 Eq) at 0° C. The reaction mixture was stirred at RT for 2 h then was quenched with ice-cold water and extracted with DCM. The combined organic layers were dried with sodium sulphate, filtered and concentrated in vacuo. The resulting residue was purified by silica gel column chromatography (gradient=2% MeOH in DCM). The appropriate fractions were concentrated in vacuo to afford tert-butyl 5-(hydroxymethyl)-1-methyl-3,4-dihydroisoquinoline-2(1H)-carboxylate (450 mg, 1.61 mmol, 88% yield) as white solid. LCMS m/z=178.4 [M-Boc+H]⁺.

163: To a stirred solution of tert-butyl 5-(hydroxymethyl)-1-methyl-3,4-dihydroisoquinoline-2(1H)-carboxylate 1 (200 mg, 0.72 mmol, 1.0 Eq) in DCM (5 mL) at 0° C. was added CBr₄ (359 mg, 1.08 mmol, 1.5 Eq) and triphenylphosphine (284 mg, 1.082 mmol, 1.5 Eq). The reaction was stirred for 2 h at RT before being quenched with ice-cold water and extracted with DCM. The combined organic layers were dried with sodium sulphate, filtered and concentrated in vacuo. The resulting residue was purified by silica gel column chromatography (gradient=8-10% EtOAc in hexane). The appropriate fractions were concentrated in vacuo to afford tert-butyl 5-(bromomethyl)-1-methyl-3,4-dihydroisoquinoline-2(1H)-carboxylate (210 mg, 0.59 mmol, 82% yield). LCMS m/z=240.2 [M−100+H]⁺.

164: To a stirred solution of benzyl 3-oxopiperazine-1-carboxylate (275 mg, 1.18 mmol 1.0 Eq) in THF (8 mL) at 0° C. was added NaH (60%, 47 mg, 1.18 mmol, 2.0 Eq). After 5 min, tert-butyl 5-(bromomethyl)-1-methyl-3,4-dihydroisoquinoline-2(1H)-carboxylate (200 mg, 0.59 mmol, 2.0 Eq) was added to the reaction mixture which was stirred at room temperature for 2 h. The reaction mixture was quenched with ice-cold water and extracted with DCM. The combined organic layers were dried over anhydrous Na₂SO₄, filtered and concentrated in vacuo. The resulting residue was purified by silica gel column chromatography (gradient=6-8% EtOAc in hexane). The appropriate fractions were concentrated in vacuo to afford tert-butyl 5-((4-((benzyloxy)carbonyl)-2-oxopiperazin-1-yl) methyl)-1-methyl-3,4-dihydroisoquinoline-2(1H)-carboxylate (250 mg, 0.28 mmol, 48% yield). LCMS m/z=394.2 [M−100+H]⁺.

165: Followed general procedure 27a. Obtained 140 mg, 96% yield. LCMS m/z=304.4 [M−56+H]⁺.

166: By general procedure 1 using MP-CNBH₃ in MeOH. Obtained 130 mg, 52% yield. LCMS m/z=645.4 [M+H]⁺.

167: By general procedure 2. Obtained 110 mg, 74% yield. LCMS m/z=545.6 [M+H]⁺.

Compound B155: By general procedure 6. Obtained 6 mg, 4% yield. LCMS m/z=680.3 [M+H]⁺.

### (E/Z)-2-(5-(4-((2,6-dimethoxy-4-(1,4,5-trimethyl-6-oxo-1,6-dihydropyridin-3-yl)benzyl)(methyl)amino)piperidin-1-yl)-1-methyl-1,2,3,4-tetrahydroisoquinoline-2-carbonyl)-4,4-dimethylpent-2-enenitrile (B33)

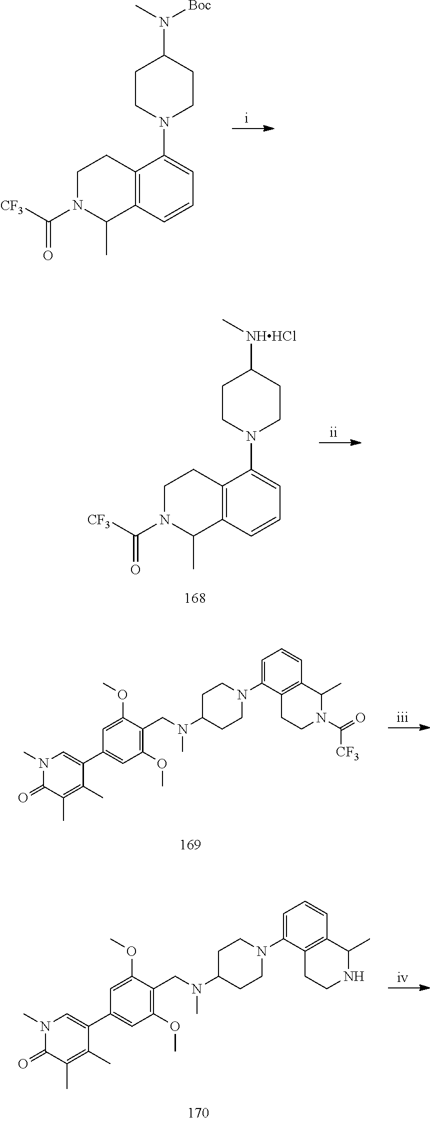

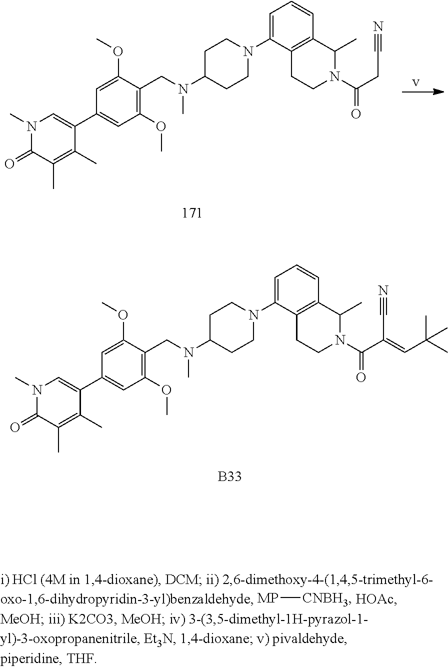

168: By general procedure 2. Obtained 1.2 g, 99% yield. LCMS m/z=358.3 [M+H]⁺.

169: By general procedure 1 using MP-CNBH₃ in DCE and MeOH. Obtained 750 mg, 74% yield. LCMS m/z=641.6 [M+H]⁺.

170: To a stirred solution of 5-(3,5-dimethoxy-4-((methyl(1-(1-methyl-2-(2,2,2-trifluoroacetyl)-1,2,3,4-tetrahydroisoquinolin-5-yr)piperidin-4-yl)amino)methyl)phenyl)-1,3,4-trimethylpyridin-2(1H)-one (750 mg, 1.171 mmol) in methanol (5 mL) and water (1.2 mL), was added K₂CO₃ (809 mg, 5.85 mmol). The reaction mixture was stirred at RT for 12 h before being filtered and the filtrate washed with methanol. The filtrate was concentrated in vacuo and partitioned between water and 10% MeOH in DCM. The aqueous layer was washed with 10% MeOH in DCM. The combined organic layers were then dried over Na₂SO₄, filtered and concentrated in vacuo to obtain 5-(3,5-dimethoxy-4-((methyl(1-(1-methyl-1,2,3,4-tetrahydroisoquinolin-5-yl)piperidin-4-yl)amino)methyl)phenyl)-1,3,4-trimethylpyridin-2(1H)-one (500 mg, 0.90 mmol, 77% yield). LCMS m/z=545.3 [M+H]⁺.

171: By general procedure 3. Obtained 490 mg, 99% yield. LCMS m/z=610.5 [M+H]⁺.

Compound B33: By general procedure 4. Obtained 17 mg, 10% yield. LCMS m/z=880.4 [M+H]⁺.

### Compound B180

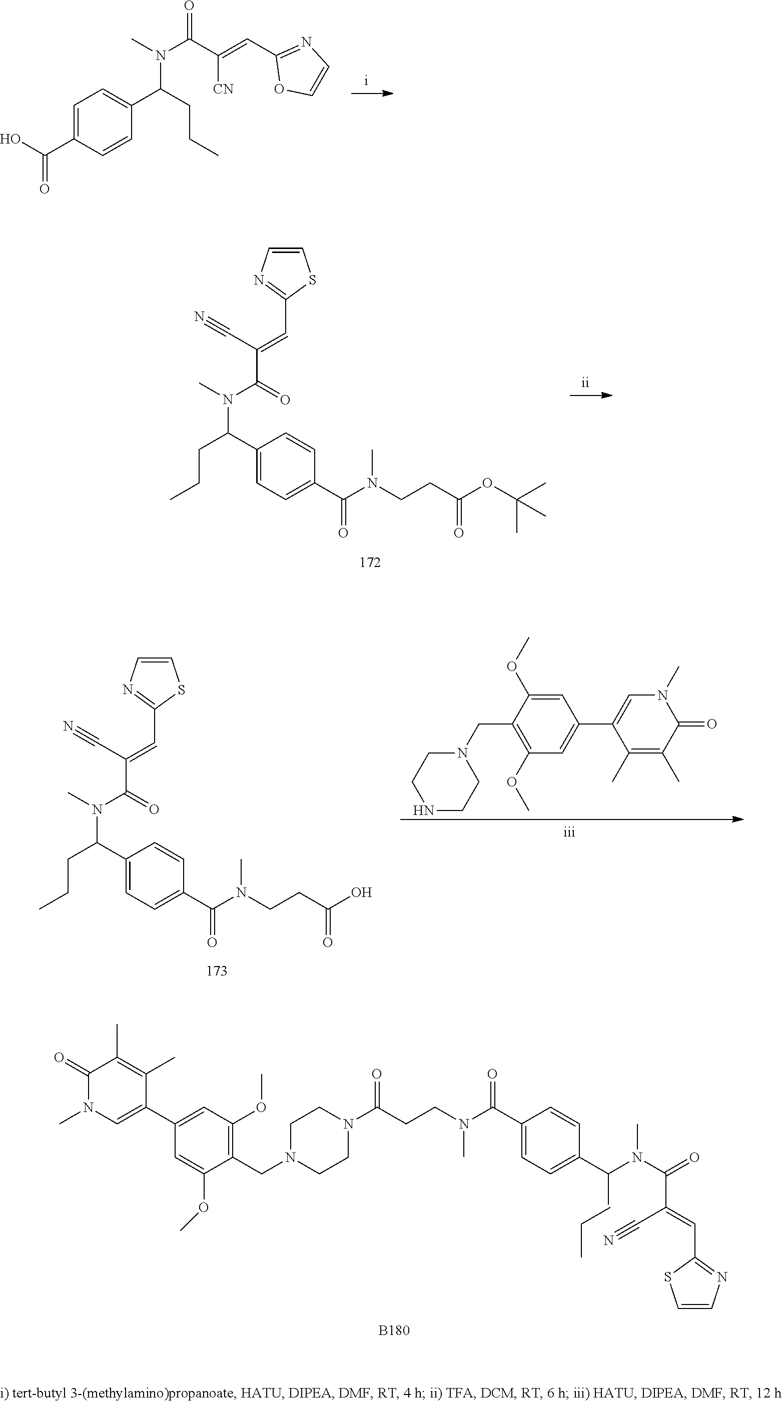

172: To a stirring solution of (E/Z)-4-(1-(2-cyano-N-methyl-3-(thiazol-2-yl)acrylamido)butyl)benzoic acid (3.0 g, 8.13 mmol) in DMF (15 mL) were added tert-butyl 3-(methylamino)propanoate (1.55 g, 9.75 mmol), HATU (4.6 g, 12.19 mmol) and DIPEA (2.83 mL, 16.26 mmol) at room temperature. The reaction mixture was stirred at room temperature for 4 h. The reaction was monitored by TLC; after completion of the reaction, the reaction mixture was diluted with EtOAc and washed with water followed by brine. The combined organic layers were dried over anhydrous Na₂SO₄, filtered and concentrated under reduced pressure. The crude material was purified by medium pressure liquid chromatography (gradient=5-10% EtOAc in heptane) to afford tert-butyl (E)-3-(4-(1-(2-cyano-N-methyl-3-(thiazol-2-yl)acrylamido)butyl)-N-methylbenzamido)propanoate (3.3 g, 79.7%) as an off white solid.

LCMS m/z=511.45 [M+H]⁺

173: By general procedure 7. Method A. Obtained 1.8 g, 67% yield.

LCMS m/z=455.2 [M+H]⁺

Compound B180: By general procedure 6. Obtained 2.81 mg, 16% yield. LCMS m/z=808.56 [M+H]⁺

### (E/Z)-4-(1-(2-cyano-N-methyl-3-(thiazol-2-yl)acrylamido)butyl)-N-(5-(4-(2,6-dimethoxy-4-(1,4,5-trimethyl-6-oxo-1,6-dihydropyridin-3-yl)benzyl)piperazin-1-yl)-5-oxopentyl)-N-methylbenzamide (B181)

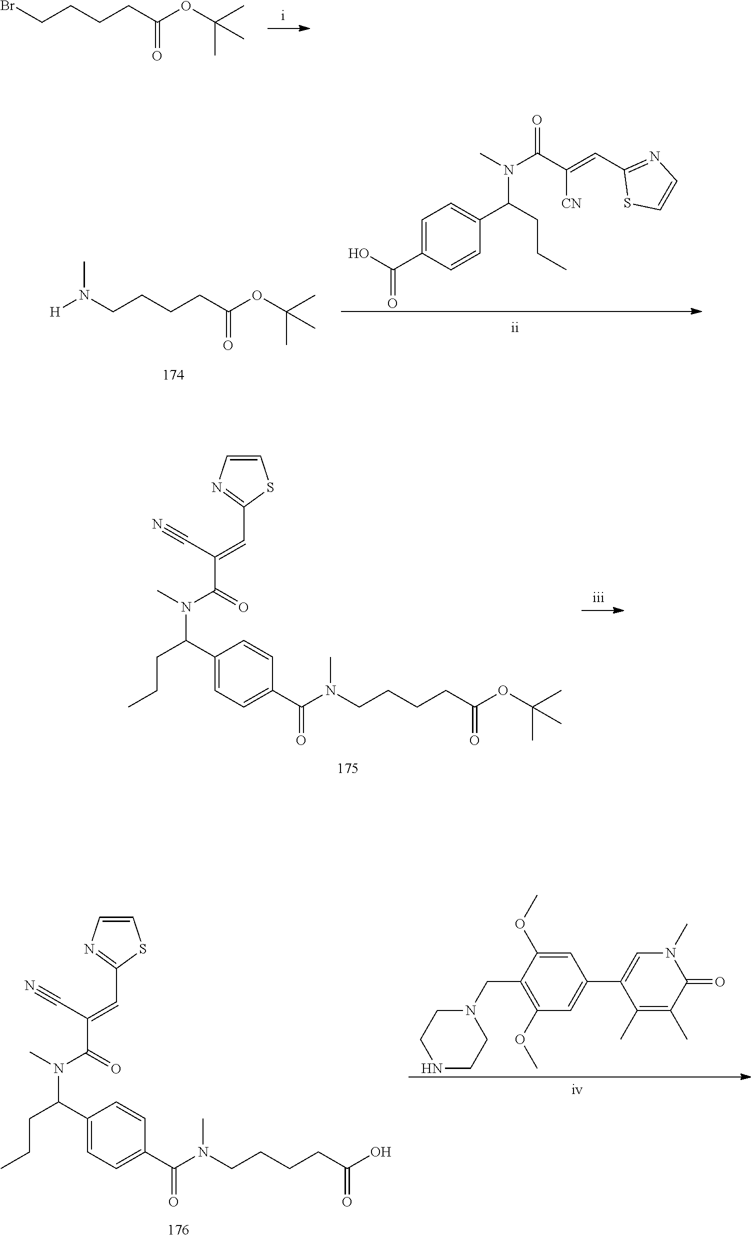

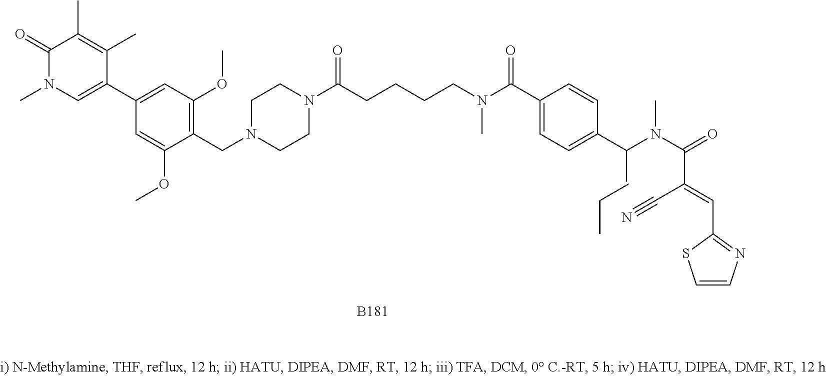

175: By general procedure 6. Obtained 2 g, 68% yield. LC-MS: m/z=539.2 [M+H]⁺

176: By general procedure 7. Obtained 550 mg, 28% yield.

LC-MS: m/z=483.4 [M+H]⁺

Compound B181: By general procedure 6. Obtained 1.26 mg, 7% yield. LCMS m/z=838.83 [M+H]⁺

### (E/Z)-4-(1-(2-cyano-N-methyl-3-(thiazol-2-yl)acrylamido)butyl)-N-(7-(4-(2,6-dimethoxy-4-(1,4,5-trimethyl-6-oxo-1,6-dihydropyridin-3-yl)benzyl)piperazin-1-yl)-7-oxoheptyl)-N-methylbenzamide (B182)

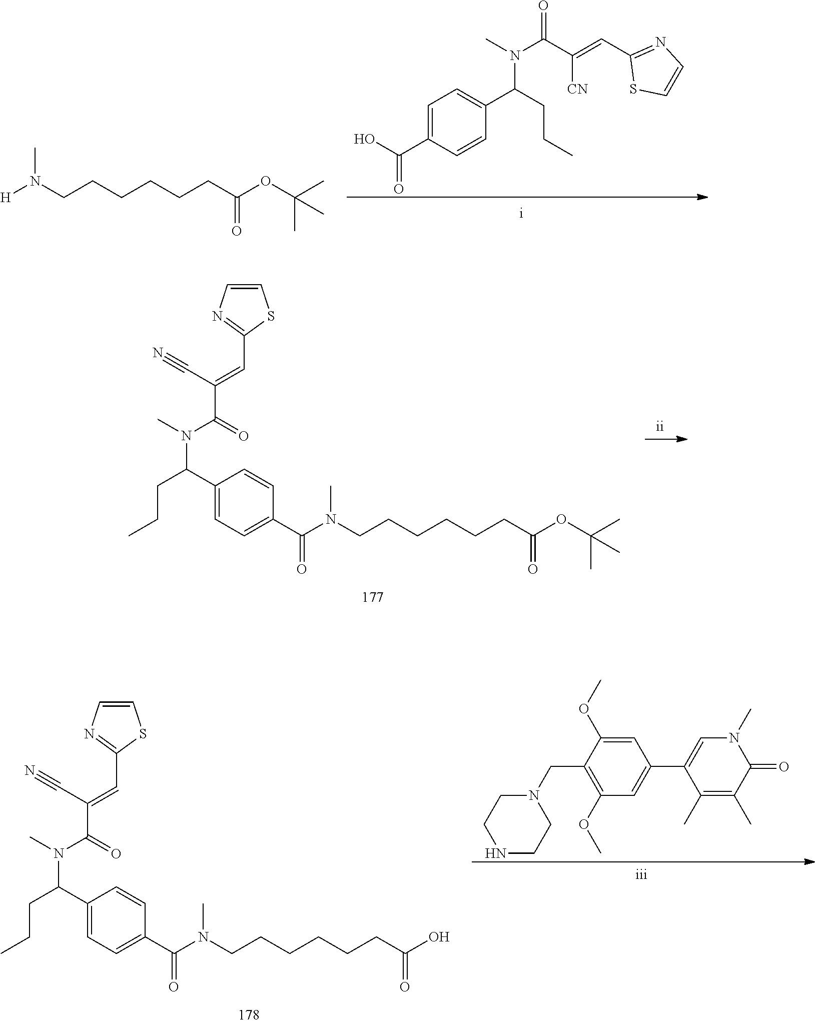

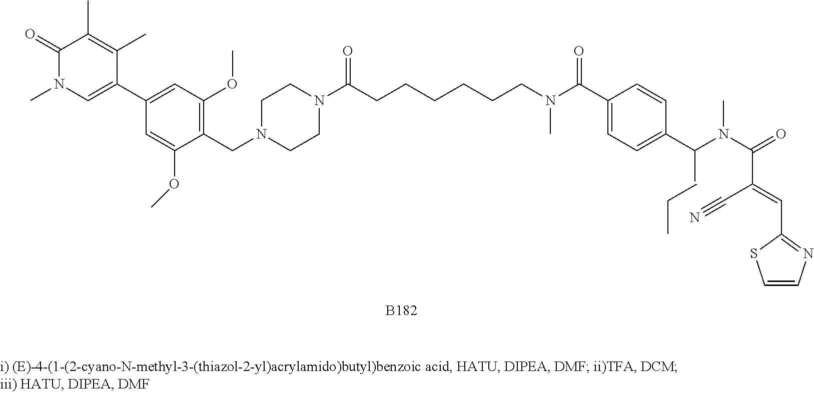

177: By general procedure 6. Obtained 1.5 g, 48% yield. LCMS m/z=511.8 [M−56+H]⁺

178: By general procedure 7. Obtained 750 mg, 55% yield. LCMS m/z=511.2 [M+H]⁺

Compound 182: By general procedure 6. Obtained 1.41 mg, 8% yield. LCMS m/z=864.9 [M+H]⁺

### (E/Z)-4-(1-(2-cyano-N-methyl-3-(thiazol-2-yl)acrylamido)butyl)-N-(9-(4-(2,6-dimethoxy-4-(1,4,5-trimethyl-6-oxo-1,6-dihydropyridin-3-yl)benzyl)piperazin-1-yl)-9-oxononyl)-N-methylbenzamide (B183)

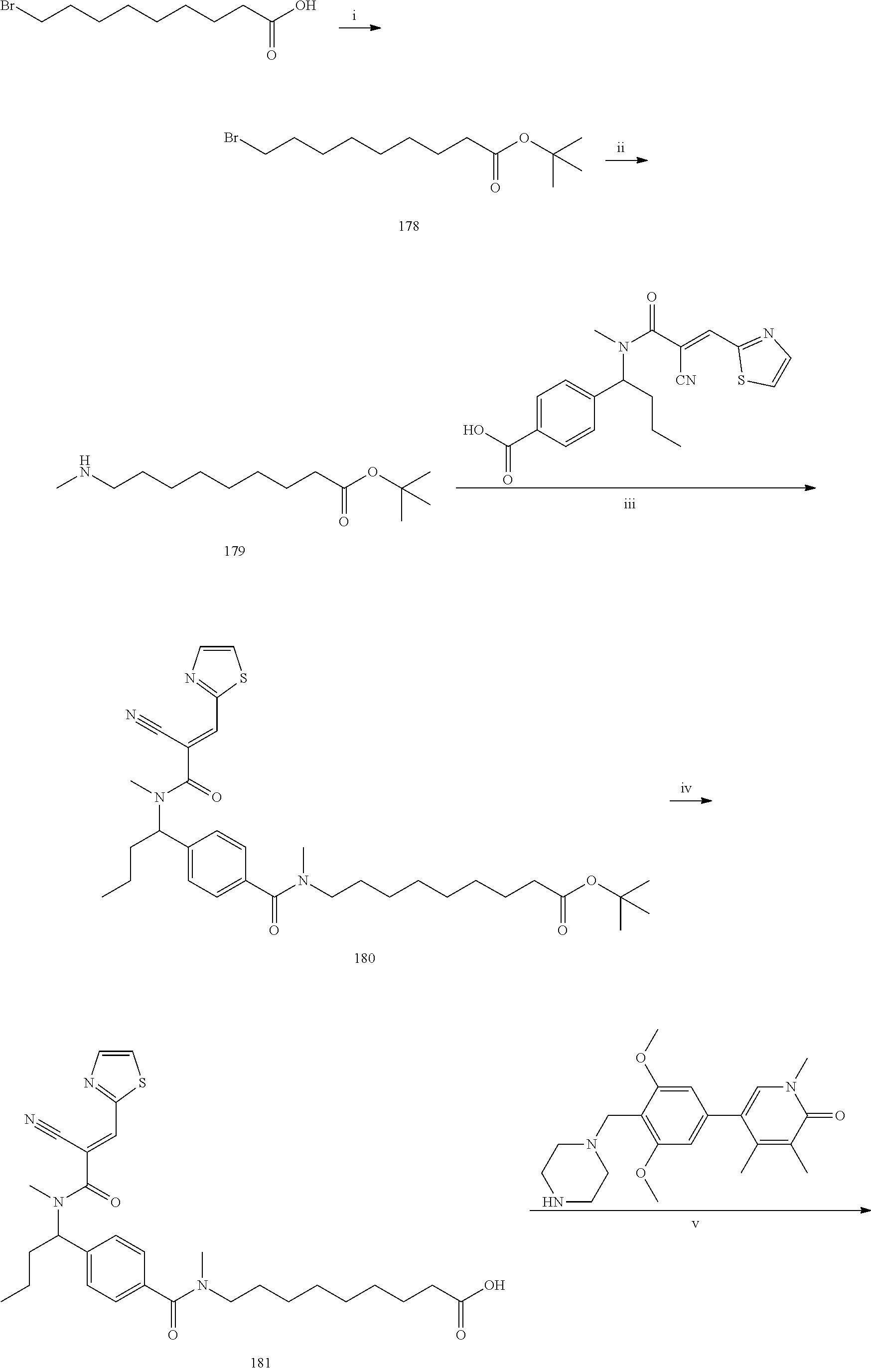

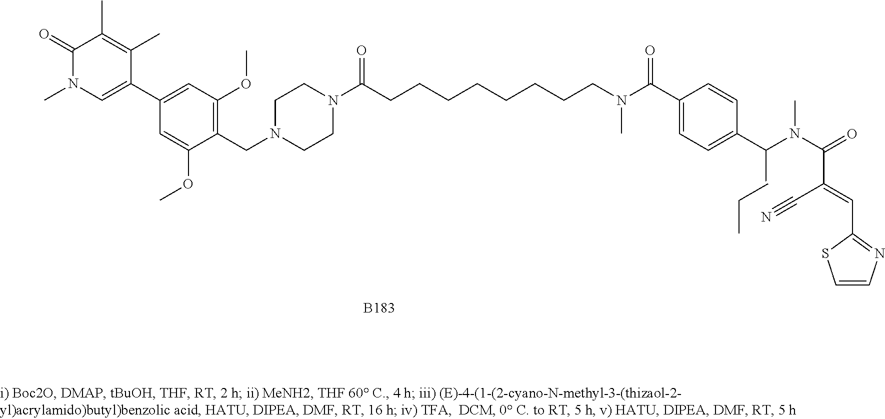

179: To a stirring solution of tert-butyl 9-bromononanoate (1.3 g, 4.43 mmol) in THF (30 mL) was added MeNH₂ (2.0 M in THF) (22 mL, 44.38 mmol) at room temperature. The reaction mixture was heated at 60° C. for 4 h. The reaction was monitored by TLC: after completion of the reaction, the reaction mixture was concentrated. The crude was purified by column chromatography by eluting with 2-5% MeOH in DCM to afford tert-butyl 9-(methylamino)nonanoate (500 mg, 48.7%) as pale yellow liquid. LC-MS: 244.2 [M+H]⁺

180: By general procedure 8. Obtained 1 g, 31% yield. LC-MS: m/z=595.4 [M+H]⁺

181: By general procedure 7 Method A. Obtained 1 g, 51% yield. LC-MS: m/z=539.2 [M+H]⁺

Compound B183: By general procedure 6. Obtained 3.11 mg, 18% yield. LCMS m/z=892.97 [M+H]⁺

### (E/Z)-2-(2-(3-(4-(2,6-dimethoxy-4-(1,4,5-trimethyl-6-oxo-1,6-dihydropyridin-3-yl)benzyl)piperazine-1-carbonyl)phenyl)pyrrolidine-1-carbonyl)-3-(thiazol-2-yl)acrylonitrile (B22)

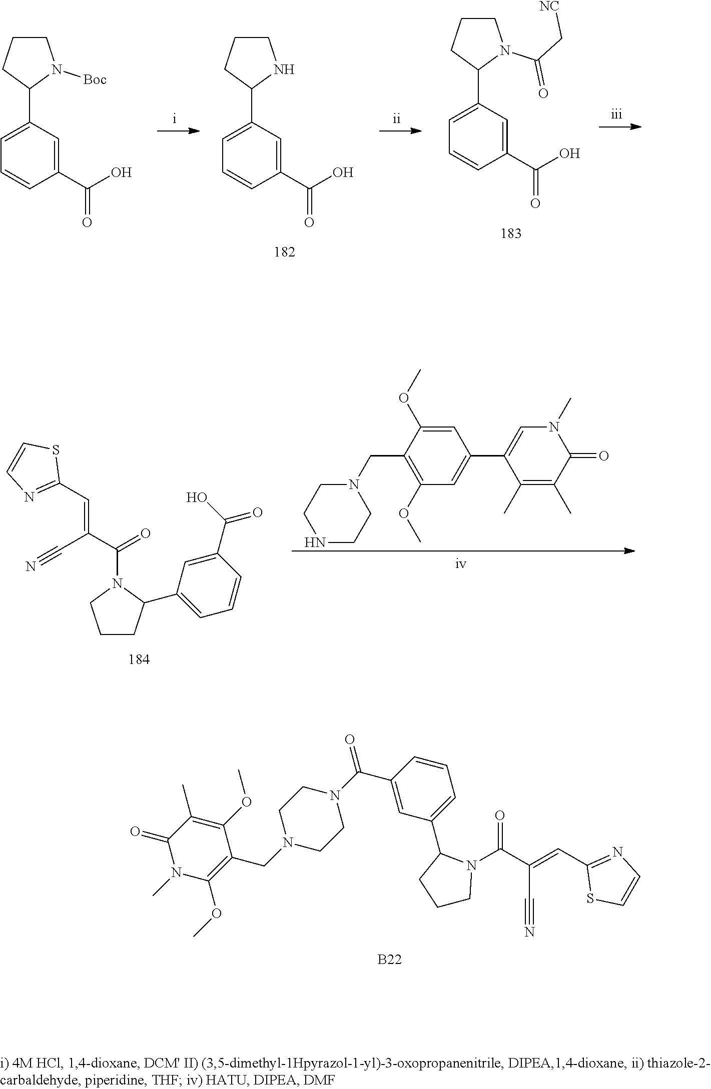

182: By general procedure 2a using starting material described in WO2012/006202. Obtained 3.8 g. LCMS m/z=192.0 [M+H]⁺

183: By general procedure 6. Obtained 1.8 g. LCMS m/z=259.2 [M+H]⁺

184: By general procedure 4. Obtained 1.7 g, 69% yield. LCMS m/z=354.2 [M+H]⁺

Compound B22: By general procedure 6. Obtained 2.22 mg, 11% yield. LCMS m/z=707.2 [M+H]⁺

### (E/Z)—N-(2-(3-(1-(2-cyano-3-(thiazol-2-yl)acryloyl)pyrrolidin-2-yl)phenoxy)ethyl)-2-(4-(2,6-dimethoxy-4-(1,4,5-trimethyl-6-oxo-1,6-dihydropyridin-3-yl)benzyl)piperazin-1-yl)acetamide (B32)

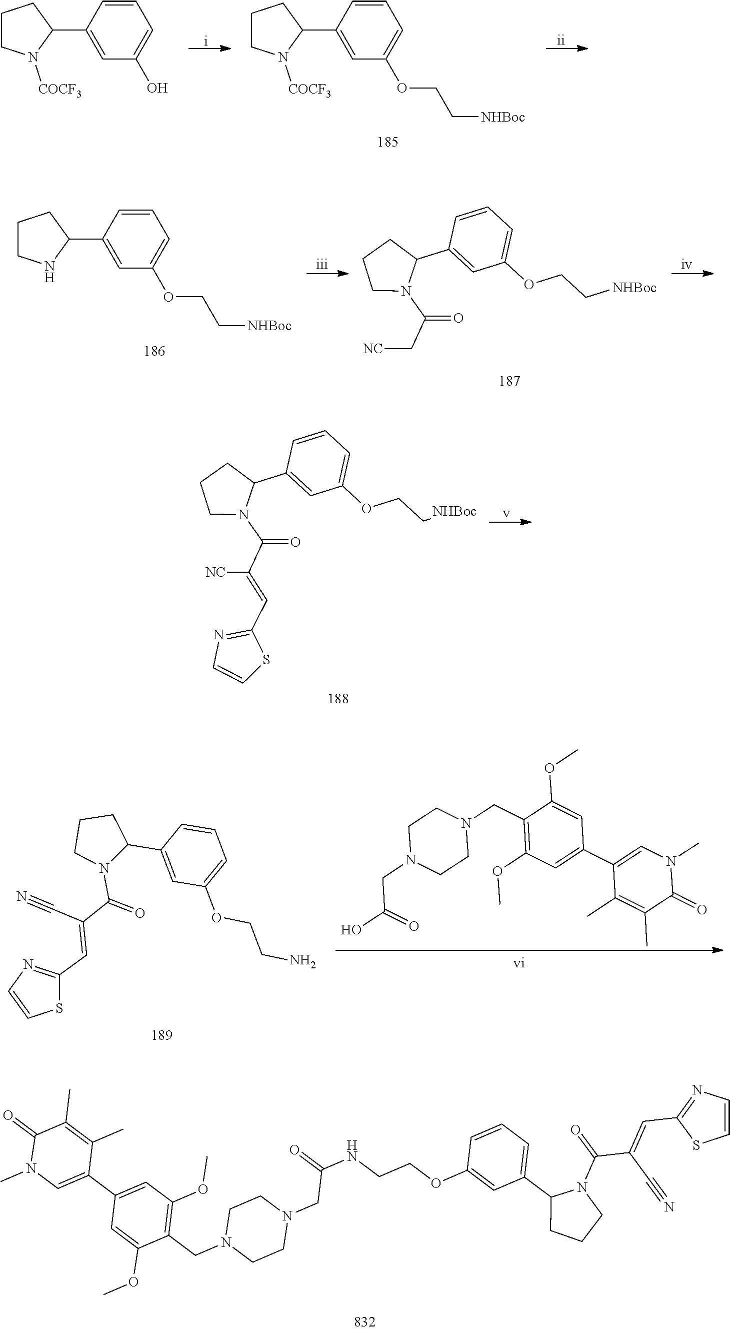

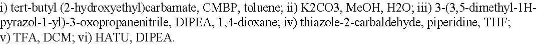

185: To a stirring solution of 2,2,2-trifluoro-1-[2-(3-hydroxyphenyl)pyrrolidin-1-yl]ethanone (3.50 g, 13.50 mmol) in toluene (30 mL) were added tert-butyl N-(2-hydroxyethyl)carbamate (2.61 g, 16.20 mmol) and CMBP (Trunoda reagent) (4.88 g, 20.25 mmol) at room temperature. The reaction mixture was heated at 80° C. for 12 h. The reaction was monitored by TLC; after completion, the reaction mixture was concentrated under reduced pressure. The resulting crude was diluted with water (70 mL) and extracted with EtOAc (3×100 mL) The combined organic layers were dried over anhydrous Na₂SO₄, filtered and concentrated under reduced pressure. The crude material was purified by medium pressure liquid chromatography (gradient=20-25% EtOAc in heptane) to afford tert-butyl N-[2-[3-[1-(2,2,2-trifluoroacetyl)pyrrolidin-2-yl]phenoxy]ethyl]carbamate (2.50 g, 46.04%) as pale green liquid. LC-MS: m/z=349.2 [M-56+H]⁺

186: Followed general procedure 20. The crude was diluted with water (80 mL) and extracted with EtOAc (2×100 mL). The combined organic layers were dried over anhydrous Na₂SO₄, filtered and concentrated under reduced pressure. Obtained 1.8 g, 85%. LC-MS: m/z=307.2 [M+H]⁺

187: By general procedure 6. Obtained 2.2 g, 78% yield. LC-MS: m/z=374.31 [M+H]⁺

188: By general procedure 4. Obtained 1.5 g, 54% yield.

¹H NMR (400 MHz, DMSO-d₆) δ=8.28-8.05 (m, 1H), 7.68 (d, J=3.9 Hz, 1H), 7.28-7.13 (m, 1H), 6.99 (br s, 1H), 6.91-6.65 (m, 3H), 4.08-3.66 (m, 4H), 2.37 (d, J=6.4 Hz, 4H), 1.91 (d, J=5.9 Hz, 2H), 1.75 (d, J=5.9 Hz, 2H), 1.49-1.12 (m, 9H)

LC-MS: m/z=469.34 [M+H]⁺

189: By general procedure 13. Obtained 750 mg, 94% yield. LC-MS: in/z=369.2 [M+H]⁺

Compound B32: By general procedure B. Obtained 3.1 mg, 14% yield.

LCMS m/z=780.9 [M+H]⁺

### (E/Z)-2-(7-(2-(2-(4-(2,6-dimethoxy-4-(1,4,5-trimethyl-6-oxo-1,6-dihydropyridin-3-yl)benzyl)piperazin-1-yl)-2-oxoethoxy)ethoxy)-1-methyl-1,2,3,4-tetrahydroisoquinoline-2-carbonyl)-3-(thiazol-2-yl)acrylonitrile (B82)

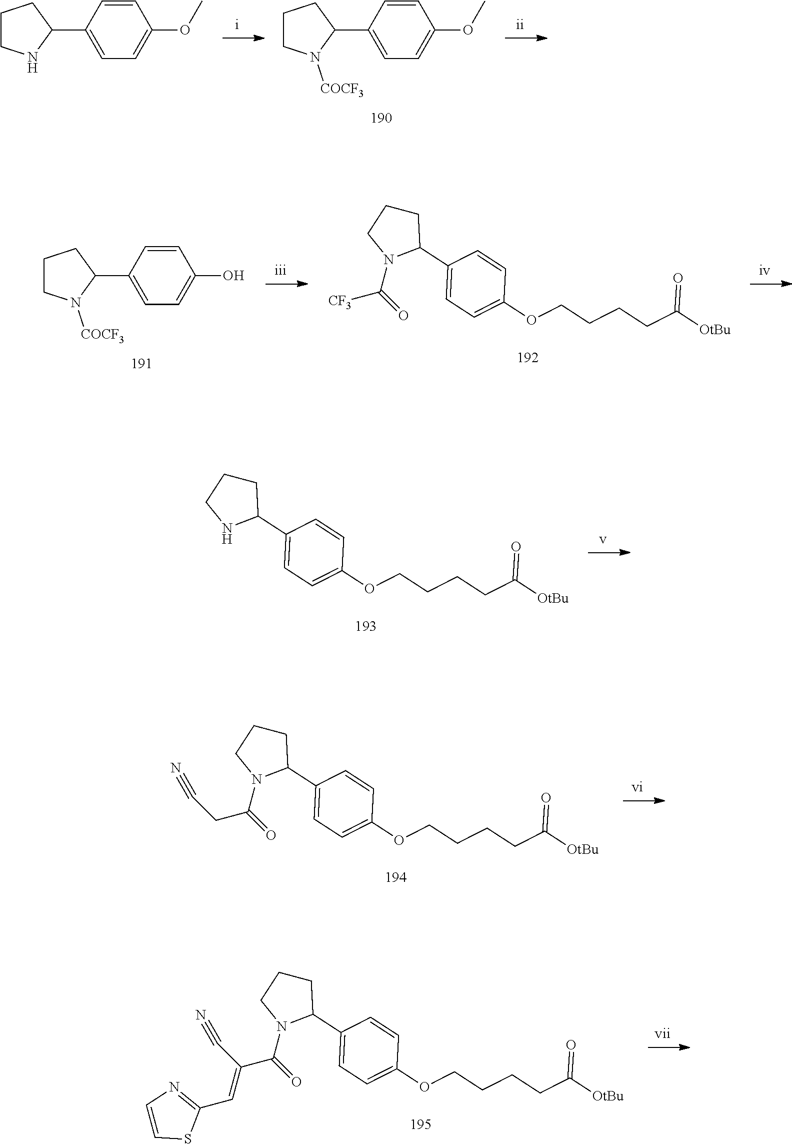

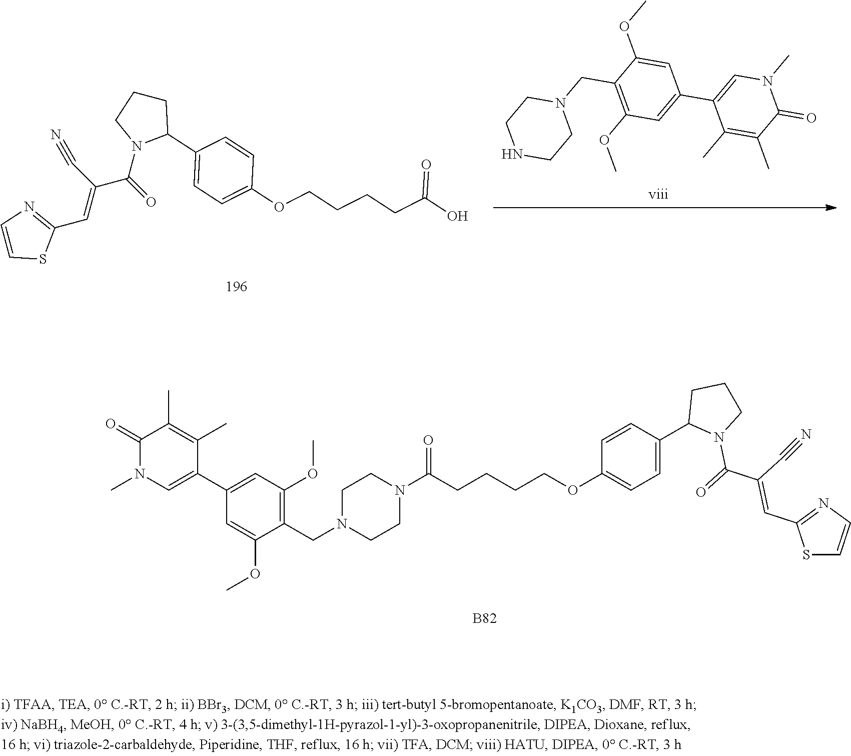

191: To a stirring solution of 2,2,2-trifluoro-1-(2-(4-methoxyphenyl)pyrrolidin-1-yl)ethan-1-one (4 g, 14.85 mmol) in DCM (40 mL) was added BBr₃ (2.1 mL, 21.8 mmol) at 0° C. The reaction mixture was stirred at room temperature for 3 h. The reaction was monitored by TLC; after completion of the reaction, the reaction mixture was quenched with methanol and extracted with DCM. The combined organic layers were dried over anhydrous Na₂SO₄, filtered and concentrated under reduced pressure. The crude material was purified by medium pressure liquid chromatography (gradient=5-10% EtOAc in heptane) to afford 2,2,2-trifluoro-1-(2-(4-hydroxyphenyl)pyrrolidin-1-yl)ethan-1-one (1 g, 26%) as yellow semi solid. LCMS m/z=260.2 [M+H]⁺

192: To a stirring solution of 2,2,2-trifluoro-1-(2-(4-hydroxyphenyl)pyrrolidin-1-yl)ethan-1-one (4 g, 15.4 mmol) in DMF (40 mL) was added tert-butyl 5-bromopentanoate (4.39 g, 18.5 mmol) followed by K₂CO₃ (6.39 g, 46.2 mmol) at room temperature and stirred for 3 h. The reaction was monitored by TLC; after completion of the reaction, the reaction mixture was quenched with ice cooled water, and solid was precipitated. The precipitate was filtered and the filtrate was dried under vacuum to afford tert-butyl 5-(4-(1-(2,2,2-trifluoroacetyl)pyrrolidin-2-yl)phenoxy)pentanoate (4 g, 63%) as white solid.

LCMS m/z=360.32 [M-56+H]⁺

193: To a stirring solution of tert-butyl 5-(4-(1-(2,2,2-trifluoroacetyl)pyrrolidin-2-yl)phenoxy)pentanoate (4 g, 9.62 mmol) in methanol (50 mL) was added NaBH₄ (2.18 g, 57.76 mmol) at 0° C. The reaction mixture was allowed to warm to room temperature and stirred for 4 h. The reaction was monitored by TLC; after completion of the reaction, the reaction mixture was evaporated under reduced pressure. The residue was diluted in water and extracted with ethyl acetate. The combined organic layers were dried over anhydrous Na₂SO₄, filtered and concentrated under reduced pressure to afford tert-butyl 5-(4-(pyrrolidin-2-yl)phenoxy)pentanoate (4 g, crude) as white solid. This material was used in next step without any further purification.

LCMS m/z=320.3 [M+H]⁺

194: By general procedure 6. Obtained 2.3 g, 48% yield. LCMS m/z=331.35 [M−56+H]⁺

195: By general procedure 4. Obtained 1.8 g, 66% yield. LCMS m/z=426.29 [M−56+H]⁺

197: By general procedure 7 Method A. Obtained 0.42 g, 59% yield. LCMS m/z=426.32 [M+H]⁺

Compound B82: By general procedure 6. Obtained 1.03 mg, 5% yield. LCMS m/z=779.4 [M+H]⁺

### Compound B185: (E/Z)-2-cyano-N-(1-(3-(3-(4-(2,6-dimethoxy-4-(1,4,5-trimethyl-6-oxo-1,6-dihydropyridin-3-yl)benzyl)piperazin-1-yl)-3-oxopropyl)phenyl)butyl)-N-methy-3-(thiazol-2-yl)acrylamide

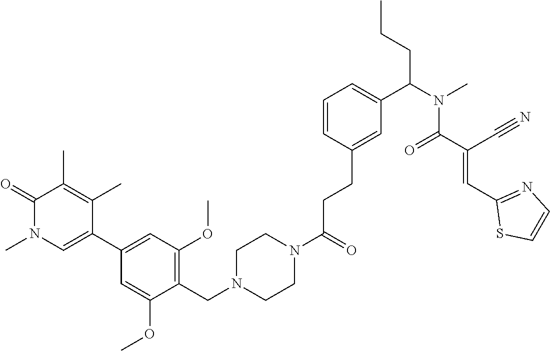 [SMILES: CCCC(c1cccc(CCC(=O)N2CCN(Cc3c(OC)cc(-c4cn(C)c(=O)c(C)c4C)cc3OC)CC2)c1)N(C)C(=O)/C(C#N)=C/c1nccs1]

Prepared following general procedure 4 using Intermediate W-2 and the amine as in Table 4a. Obtained 4.4 mg, 23% yield. LCMS m/z=751.7 [M+H]⁺

### Compound B184: (E/Z)-2-cyano-N-(1-(3-(4-(2,6-dimethoxy-4-(1,4,5-trimethyl-6-oxo-1,6-dihydropyridin-3-yl)benzyl)piperazine-1-carbonyl)phenyl)butyl)-N-methyl-3-(thiazol-2-yl)acrylamide

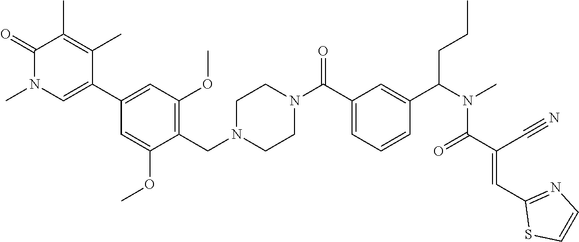 [SMILES: CCCC(c1cccc(C(=O)N2CCN(Cc3c(OC)cc(-c4cn(C)c(=O)c(C)c4C)cc3OC)CC2)c1)N(C)C(=O)/C(C#N)=C/c1nccs1]

Prepared following general procedure 6 using Intermediate W-3 and the amine as in Table 4a. Obtained 2.2 mg, 11% yield. LCMS m/z=723.7 [M+H]⁺

### Compound B186: (E/Z)-2-cyano-N-(1-(2-(3-(4-(2,6-dimethoxy-4-(1,4,5-trimethyl-6-oxo-1,6-dihydropyridin-3-yl)benzyl)piperazin-1-yl)-3-oxopropyl)phenyl)butyl)-N-methyl-3-(thiazol-2-yl)acrylamide

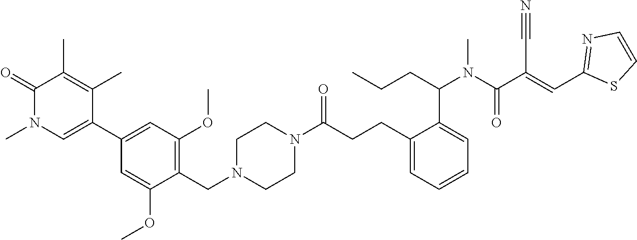 [SMILES: CCCC(c1ccccc1CCC(=O)N1CCN(Cc2c(OC)cc(-c3cn(C)c(=O)c(C)c3C)cc2OC)CC1)N(C)C(=O)/C(C#N)=C/c1nccs1]

Prepared following general procedure 6 using Intermediate W-4 and the amine as in Table 4a. Obtained 2.87 mg, 15% yield. LCMS m/z=751.7 [M+H]⁺

### Compound B31: (E/Z)—N-(2-((2-(2-cyano-3-(thiazol-2-yl)acryloyl)-1-methyl-1,2,3,4-tetrahydroisoquinolin-6-yl)oxy)ethyl)-2-(4-(2,6-dimethoxy-4-(1,4,5-trimethyl-1-oxo-1,6-dihydropyridin-3-yl)benzyl)piperazin-1-yl)acetamide

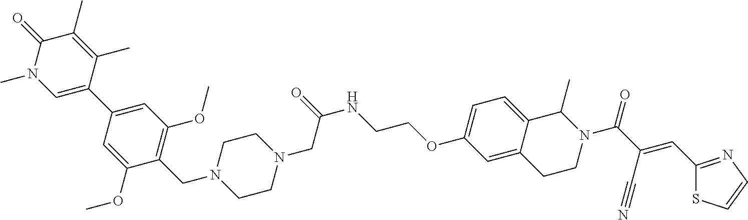 [SMILES: COc1cc(-c2cn(C)c(=O)c(C)c2C)cc(OC)c1CN1CCN(CC(=O)NCCOc2ccc3c(c2)CCN(C(=O)/C(C#N)=C/c2nccs2)C3C)CC1]

Prepared following general procedure 6 using Intermediate W-6 (alkylated using standard conditions with bromoacetic acid) and the amine as in Table 4a. Obtained 2.01 mg, 12% yield. LCMS m/z=780.4 [M+H]⁺

### Compound B83: (E/Z)-2-(7-(2-(2-(4-(2,6-dimethoxy-4-(1,4,5-trimethyl-6-oxo-1,6-dihydropyridin-3-yl)benzyl)piperazin-1-yl)-2-oxoethoxy)ethoxy)-1-methyl-1,2,3,4-tetrahydroisoquinoline-2-carbonyl)-3-(thiazol-2-yl)acrylonitrile

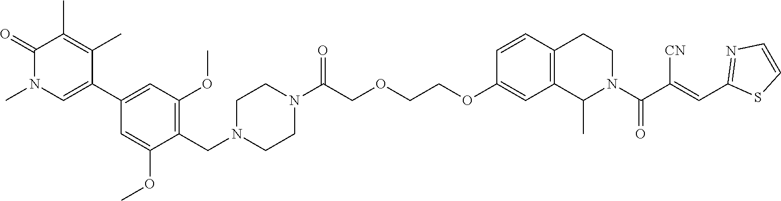 [SMILES: COc1cc(-c2cn(C)c(=O)c(C)c2C)cc(OC)c1CN1CCN(C(=O)COCCOc2ccc3c(c2)C(C)N(C(=O)/C(C#N)=C/c2nccs2)CC3)CC1]

Prepared following general procedure 6 using Intermediate W-8 and the amine as in Table 4a. Obtained 4.1 mg, 2% yield. LCMS m/z=781.7 [M+H]⁺

### Compound B84: (E/Z)-2-(7-((5-(4-(2,6-dimethoxy-4-(1,4,5-trimethyl-6-oxo-1,6-dihydropyridin-3-yl)benzyl)piperazin-1-yl)-5-oxopentyl)oxy)-1-methyl-1,2,3,4-tetrahydroisoquinoline-2-carbonyl)-3-(thiazol-2-yl)acrylonitrile

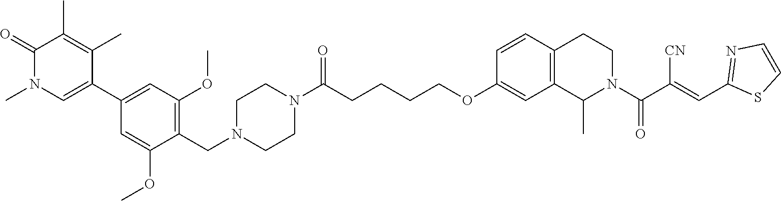 [SMILES: COc1cc(-c2cn(C)c(=O)c(C)c2C)cc(OC)c1CN1CCN(C(=O)CCCCOc2ccc3c(c2)C(C)N(C(=O)/C(C#N)=C/c2nccs2)CC3)CC1]

Prepared following general procedure 6 using Intermediate W-9 and the amine as in Table 4a. Obtained 2.3 mg, 11% yield. LCMS m/z=779.0 [M+H]⁺

### Compound B29: (E/Z)—N-(2-((2-(2-cyano-3-(thiazol-2-yl)acryloyl)-1-methyl-1,2,3,4-tetrahydroisoquinolin-7-yl)oxy)ethyl)-2-(4-(2,6-dimethoxy-4-(1,4,5-trimethyl-6-oxo-1,6-dihydropyridin-3-yl)benzyl)piperazin-1-yl)acetamide

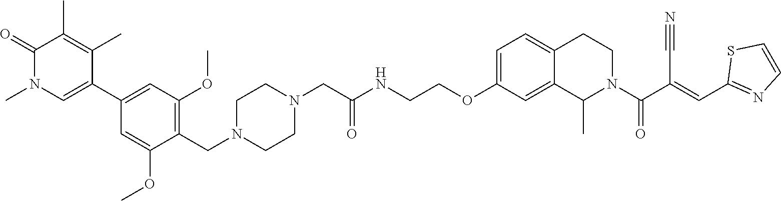 [SMILES: COc1cc(-c2cn(C)c(=O)c(C)c2C)cc(OC)c1CN1CCN(CC(=O)NCCOc2ccc3c(c2)C(C)N(C(=O)/C(C#N)=C/c2nccs2)CC3)CC1]

Prepared following general procedure 6 using Intermediate W-10 (alkylated using standard procedures with bromoacetic acid) and the amine as in Table 4a. Obtained 1.09 mg, 5% yield. LCMS m/z=779.9 [M+H]⁺

### Compound B30: (E/Z)—N-(2-((2-(2-cyano-3-(thiazol-2-yl)acryloyl)-1-methyl-1,2,3,4-tetrahydroisoquinolin-8-yl)oxy)ethyl)-2-(4-(2,6-dimethoxy-4-(1,4,5-trimethyl-6-oxo-1,6-dihydropyridin-3-yl)benzyl)piperazin-1-yl)acetamide

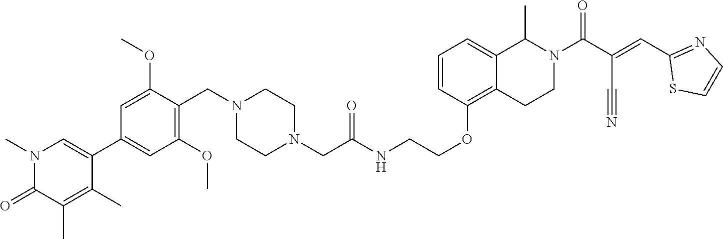 [SMILES: COc1cc(-c2cn(C)c(=O)c(C)c2C)cc(OC)c1CN1CCN(CC(=O)NCCOc2cccc3c2CCN(C(=O)/C(C#N)=C/c2nccs2)C3C)CC1]

Prepared following general procedure 6 using Intermediate W-11 (alkylated using standard procedures with bromoacetic acid) and the amine as in Table 4a. Obtained 1.42 mg, 6% yield. LCMS m/z=780.4 [M+H]⁺

### Intermediate W-31: 3-(5-((7-(2,6-dimethoxy-4-(1,4,5-trimethyl-6-oxo-1,6-dihydropyridin-3-yl)benzyl)-4,7-diazaspiro[2.5]octan-4-yl)methyl)-1-methyl-3,4-dihydroisoquinolin-2(1H)-yl)-3-oxopropanenitrile

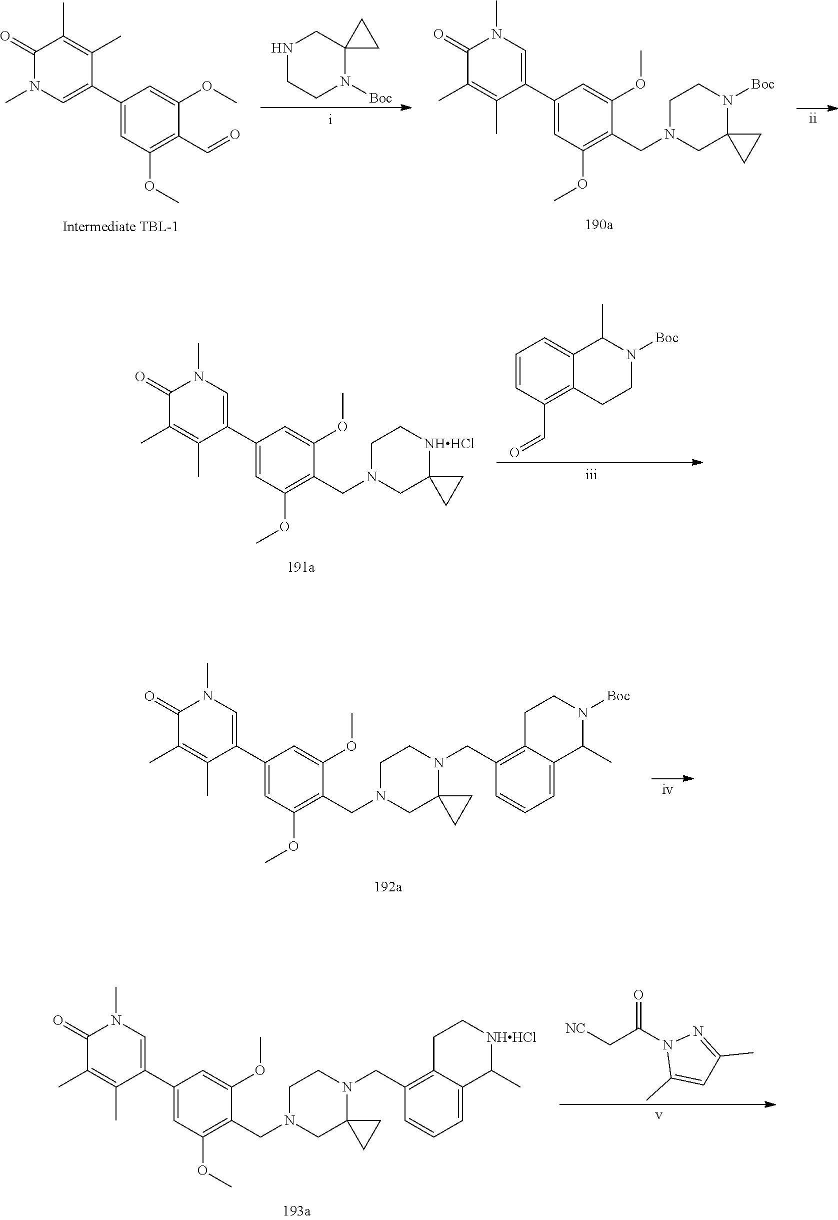

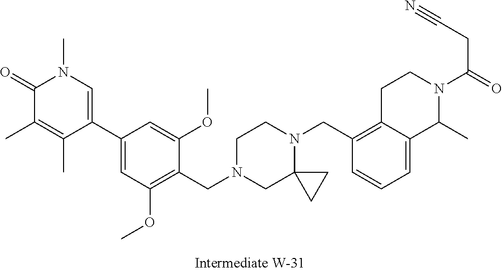 [SMILES: COc1cc(-c2cn(C)c(=O)c(C)c2C)cc(OC)c1CN1CCN(Cc2cccc3c2CCN(C(=O)CC#N)C3C)C2(CC2)C1]

190a: Prepared following general procedure 1 using MP-CNBH₃ (w/w) in MeOH. Obtained 4.1 g, 99% yield. LCMS m/z=498.2 [M+H]⁺.

191a: Prepared following general procedure 2 using HCl (4M solution in 1,4-dioxane) in DCM. Obtained 3.6 g (crude). LCMS m/z=398.4 [M+H]⁺.

192a: Prepared following general procedure 1 using MP-CNBH₃ (w/w) and acetic acid in MOH. Obtained 5 g, 82% yield. LCMS m/z=657.3 [M+H]⁺.

193a: Prepared following general procedure 2 using HCl (4M solution in 1,4-dioxane) in DCM. Obtained 2.4 g (crude). LCMS m/z=557.3 [M+H]⁺.

Intermediate W-31: Prepared following general procedure 3 using 3-(3,5-dimethyl-1H-pyrazol-1-yl)-3-oxopropanenitrile (1.2 equiv.) in MeCN. Obtained 2g, 65.4% yield. LCMS m/z=624.4

The following compounds were made from Intermediate W-31 and the relevant commercially-available aldehyde (or prepared as otherwise indicated) according to the relevant procedures as noted in the table below:

| | | | | LCMS |
|---|---|---|---|---|
| | | | | m/z |
| Cmpd | Name | Procedure | Yield | [M + H]⁺ |
| B174 | (E/Z)-2-(5-((7-(2,6-dimethoxy-4-(1,4,5-trimethyl-6- | 4 (using | 15.3 mg, | 720.4 |
| | oxo-1,6-dihydropyridin-3-yl)benzyl)-4,7- | aldehyde as | 11% | |
| | diazaspiro[2.5]octan-4-yl)methyl)-1-methyl-1,2,3,4- | described in | | |
| | tetrahydroisoquinoline-2-carbonyl)-3-(3- | WO2021/30711) | | |
| | methyltetrahydrofuran-3-yl)acrylonitrile | | | |
| B165 | (E/Z)-2-(5-((7-(2,6-dimethoxy-4-(1,4,5-trimethyl-6- | 4 | 9 mg, | 708.4 |
| | oxo-1,6-dihydropyridin-3-yl)benzyl)-4,7- | | 35% | |
| | diazaspiro[2.5]octan-4-yl)methyl)-1-methyl-1,2,3,4- | | | |
| | tetrahydroisoquinoline-2-carbonyl)-3-(3- | | | |
| | (trifluoromethyl)bicyclo[1.1.1]pentan-1- | | | |
| | yl)acrylonitrile | | | |
| B191 | (E/Z)-2-(5-((7-(2,6-dimethoxy-4-(1,4,5-trimethyl-6- | 4 (using | 7 mg, | 732.4 |
| | oxo-1,6-dihydropyridin-3-yl)benzyl)-4,7- | aldehyde as | 8% | |
| | diazaspiro[2.5]octan-4-yl)methyl)-1-methyl-1,2,3,4- | described in | | |
| | tetrahydroisoquinoline-2-carbonyl)-3-(3- | WO2021/124172) | | |
| | methoxybicyclo[1.1.1]pentan-1-yl)acrylonitrile | | | |
| B173 | (E/Z)-2-(5-((7-(2,6-dimethoxy-4-(1,4,5-trimethyl-6- | 4a | 15 mg, | 706.3 |
| | oxo-1,6-dihydropyridin-3-yl)benzyl)-4,7- | | 32% | |
| | diazaspiro[2.5]octan-4-yl)methyl)-1-methyl-1,2,3,4- | | | |
| | tetrahydroisoquinoline-2-carbonyl)-3- | | | |
| | (tetrahydrofuran-3-yl)acrylonitril | | | |
| B149 | (E/Z)-2-(5-((7-(2,6-dimethoxy-4-(1,4,5-trimethyl-6- | 4 | 4 mg | 704.4 |
| | oxo-1,6-dihydropyridin-3-yl)benzyl)-4,7- | | | |
| | diazaspiro[2.5]octan-4-yl)methyl)-1-methyl-1,2,3,4- | | | |
| | tetrahydroisoquinoline-2-carbonyl)-3-(1- | | | |
| | methylcyclobutyl)acrylonitrile | | | |
| B123 | (E/Z)-2-(5-((7-(2,6-dimethoxy-4-(1,4,5-trimethyl-6- | 4 | 53 mg, | 734.4 |
| | oxo-1,6-dihydropyridin-3-yl)benzyl)-4,7- | | 30% | |
| | diazaspiro[2.5]octan-4-yl)methyl)-1-methyl-1,2,3,4- | | | |
| | tetrahydroisoquinoline-2-carbonyl)-3-(4- | | | |
| | methyltetrahydro-2H-pyran-4-yl)acrylonitrile | | | |
| B124 | (E/Z)-2-(5-((7-(2,6-dimethoxy-4-(1,4,5-trimethyl-6- | 4a | 10.6 mg, | 720.4 |
| | oxo-1,6-dihydropyridin-3-yl)benzyl)-4,7- | | 18% | |
| | diazaspiro[2.5]octan-4-yl)methyl)-1-methyl-1,2,3,4- | | | |
| | tetrahydroisoquinoline-2-carbonyl)-3-(1- | | | |
| | (methoxymethyl)cyclopropyl)acrylonitrile | | | |
| B144 | (E/Z)-2-(5-((7-(2,6-dimethoxy-4-(1,4,5-trimethyl-6- | 4 | 2.7 mg, | 708.4 |
| | oxo-1,6-dihydropyridin-3-yl)benzyl)-4,7- | | 5% | |
| | diazaspiro[2.5]octan-4-yl)methyl)-1-methyl-1,2,3,4- | | | |
| | tetrahydroisoquinoline-2-carbonyl)-3-(1- | | | |
| | (fluoromethyl)cyclopropyl)acrylonitrile | | | |
| B145 | (E/Z)-2-(5-((7-(2,6-dimethoxy-4-(1,4,5-trimethyl-6- | 4 | 8 mg, | 718.4 |
| | oxo-1,6-dihydropyridin-3-yl)benzyl)-4,7- | | 14% | |
| | diazaspiro[2.5]octan-4-yl)methyl)-1-methyl-1,2,3,4- | | | |
| | tetrahydroisoquinoline-2-carbonyl)-3-(1- | | | |
| | methylcyclopentyl)acrylonitrile | | | |
| B138 | (E/Z)-2-(5-((7-(2,6-dimethoxy-4-(1,4,5-trimethyl-6- | 4 | 5 mg, | 728.4 |
| | oxo-1,6-dihydropyridin-3-yl)benzyl)-4,7- | | 8% | |
| | diazaspiro[2.5]octan-4-yl)methyl)-1-methyl-1,2,3,4- | | | |
| | tetrahydroisoquinoline-2-carbonyl)-5,5-difluoro-4,4- | | | |
| | dimethylpent-2-enenitrile | | | |
| B117 | (E/Z)-2-(5-((7-(2,6-dimethoxy-4-(1,4,5-trimethyl-6- | 4 | 6 mg, | 706.4 |
| | oxo-1,6-dihydropyridin-3-yl)benzyl)-4,7- | | 9% | |
| | diazaspiro[2.5]octan-4-yl)methyl)-1-methyl-1,2,3,4- | | | |
| | tetrahydroisoquinoline-2-carbonyl)-4,4-dimethylhex- | | | |
| | 2-enenitrile | | | |
| B125 | (E/Z)-2-(5-((7-(2,6-dimethoxy-4-(1,4,5-trimethyl-6- | 4a | 13 mg, | 710.4 |
| | oxo-1,6-dihydropyridin-3-yl)benzyl)-4,7- | | 8% | |
| | diazaspiro[2.5]octan-4-yl)methyl)-1-methyl-1,2,3,4- | | | |
| | tetrahydroisoquinoline-2-carbonyl)-5-fluoro-4,4- | | | |
| | dimethylpent-2-enenitrile | | | |
| B143 | (E/Z)-2-(5-((7-(2,6-dimethoxy-4-(1,4,5-trimethyl-6- | 4a | 56 mg, | 432.4 |
| | oxo-1,6-dihydropyridin-3-yl)benzyl)-4,7- | | 31% | |
| | diazaspiro[2.5]octan-4-yl)methyl)-1-methyl-1,2,3,4- | | | |
| | tetrahydroisoquinoline-2-carbonyl)-3-(1- | | | |
| | methylcyclohexyl)acrylonitrile | | | |
| B137 | (E/Z)-2-(5-((7-(2,6-dimethoxy-4-(1,4,5-trimethyl-6- | 4a | 48 mg, | 720.4 |
| | oxo-1,6-dihydropyridin-3-yl)benzyl)-4,7- | | 40% | |
| | diazaspiro[2.5]octan-4-yl)methyl)-1-methyl-1,2,3,4- | | | |
| | tetrahydroisoquinoline-2-carbonyl)-3-(tetrahydro- | | | |
| | 2H-pyran-4-yl)acrylonitrile | | | |
| B140 | (E/Z)-2-[5-[[7-[[2,6-dimethoxy-4-(1,4,5-trimethyl-6- | 4a | 4.5 mg, | 696.2 |
| | oxo-3-pyridyl)phenyl]methyl]-4,7- | | 32% | |
| | diazaspiro[2.5]octan-4-yl]methyl]-1-methyl-3,4- | | | |
| | dihydro-1H-isoquinoline-2-carbonyl]-4-fluoro-4- | | | |
| | methyl-pent-2-enenitrile | | | |
| B141 | (E/Z)-2-(5-((7-(2,6-dimethoxy-4-(1,4,5-trimethyl-6- | 4 | 25 mg, | 732.3 |
| | oxo-1,6-dihydropyridin-3-yl)benzyl)-4,7- | | 20% | |
| | diazaspiro[2.5]octan-4-yl)methyl)-1-methyl-1,2,3,4- | | | |
| | tetrahydroisoquinoline-2-carbonyl)-3-(3- | | | |
| | methoxybicyclo[1.1.1]pentan-1-yl)acrylonitrile | | | |
| B153 | (E/Z)-2-[5-[[7-[[2,6-dimethoxy-4-(1,4,5-trimethyl-6- | 4a | 5 mg, | 744.2 |
| | oxo-3-pyridyl)phenyl]methyl]-4,7- | | 9% | |
| | diazaspiro[2.5]octan-4-yl]methyl]-1-methyl-3,4- | | | |
| | dihydro-1H-isoquinoline-2-carbonyl]-3-[1- | | | |
| | (trifluoromethyl)cyclopropyl]prop-2-enenitrile | | | |
| B160 | (E/Z)-2-[5-[[7-[[2,6-dimethoxy-4-(1,4,5-trimethyl-6- | 4a | 38 mg, | 758.3 |
| | oxo-3-pyridyl)phenyl]methyl]-4,7- | | 16% | |
| | diazaspiro[2.5]octan-4-yl]methyl]-1-methyl-3,4- | | | |
| | dihydro-1H-isoquinoline-2-carbonyl]-3-[1- | | | |
| | (trifluoromethyl)cyclobutyl]prop-2-enenitrile | | | |
| B152 | (E/Z)-2-(5-((7-(2,6-dimethoxy-4-(1,4,5-trimethyl-6- | 4 | 9 mg, | 746.4 |
| | oxo-1,6-dihydropyridin-3-yl)benzyl)-4,7- | | 5% | |
| | diazaspiro[2.5]octan-4-yl)methyl)-1-methyl-1,2,3,4- | | | |
| | tetrahydroisoquinoline-2-carbonyl)-5,5,5-trifluoro- | | | |
| | 4,4-dimethylpent-2-enenitrile | | | |
| B164 | (E/Z)-2-[5-[[7-[[2,6-dimethoxy-4-(1,4,5-trimethyl-6- | 4a | 2 mg, | 758.3 |
| | oxo-3-pyridyl)phenylmethyl]-4,7- | | 1% | |
| | diazaspiro[2.5]octan-4-yl]methyl]-1-methyl-3,4- | | | |
| | dihydro-1H-isoquinoline-2-carbonyl]-3-(1- | | | |
| | fluorocyclobutyl)prop-2-enenitrile | | | |
| B162 | (E/Z)-3-(2-cyano-3-(5-((7-(2,6-dimethoxy-4-(1,4,5- | 4 | 16 mg, | 744.2 |
| | trimethyl-6-oxo-1,6-dihydropyridin-3-yl)benzyl)-4,7- | | 60% | |
| | diazaspiro[2.5]octan-4-yl)methyl)-1-methyl-3,4- | | | |
| | dihydroisoquinolin-2(1H)-yl)-3-oxoprop-1-en-1- | | | |
| | yl)bicyclo[1.1.1]pentane-1-carbonitrile | | | |
| B161 | (E/Z)-2-(5-((7-(2,6-dimethoxy-4-(1,4,5-trimethyl-6- | 4 | 11 mg, | 716.4 |
| | oxo-1,6-dihydropyridin-3-yl)benzyl)-4,7- | | 12% | |
| | diazaspiro[2.5]octan-4-yl)methyl)-1-methyl-1,2,3,4- | | | |
| | tetrahydroisoquinoline-2-carbonyl)-3-(3- | | | |
| | methylbicyclo[1.1.1]pentan-1-yl)acrylonitrile | | | |
| C29 | (E/Z)-2-(5-((7-(2,6-dimethoxy-4-(1,4,5-trimethyl-6- | 4a | 54 mg, | 791.2 |
| | oxo-1,6-dihydropyridin-3-yl)benzyl)-4,7- | | 24% | |
| | diazaspiro[2.5]octan-4-yl)methyl)-1-methyl-1,2,3,4- | | | |
| | tetrahydroisoquinoline-2-carbonyl)-3-(pyridin-2- | | | |
| | yl)acrylonitrile | | | |
| C30 | (E/Z)-2-(7-((7-(2,6-dimethoxy-4-(1,4,5-trimethyl-6- | 4a | 2.5 mg, | 696.3 |
| | oxo-1,6-dihydropyridin-3-yl)benzyl)-4,7- | | 3% | |
| | diazaspiro[2.5]octan-4-yl)methyl)-1-methyl-1,2,3,4- | | | |
| | tetrahydroisoquinoline-2-carbonyl)-4,4- | | | |
| | dimethylpent-2-enenitrile | | | |

### (E/Z)-2-(5-((7-(2,6-dimethoxy-4-(1,4,5-trimethyl-6-oxo-1,6-dihydropyridin-3-yl)benzyl)-4,7-diazaspiro[2.5]octan-4-yl)methyl)-1-methyl-1,2,3,4-tetrahydroisoquinoline-2-carbonyl)-3-(3-methyltetrahydrofuran-3-yl)acrylonitrile (B154)

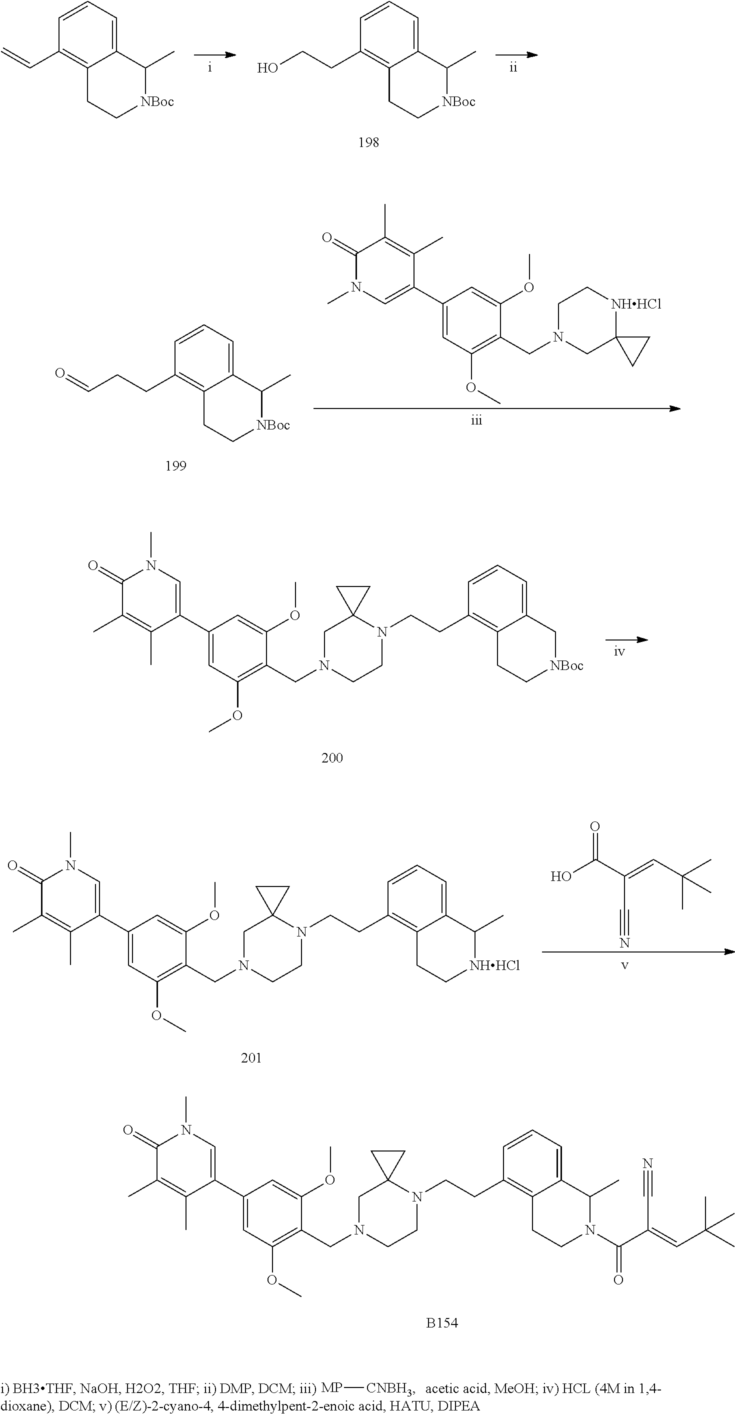

198: To a stirred solution of tert-butyl 1-methyl-5-vinyl-3,4-dihydroisoquinoline-2(1H)-carboxylate (1 g, 3.86 mmol, 1 eq) in THF (13 mL) was added BH₃·THF (1M in THF) (7.32 ml, 7.32 mmol, 2 eq) dropwise at 0° C. The resultant reaction mixture was stirred at room temperature for 3 h before being cooled to 0° C. and sodium hydroxide (2.439 ml, 7.32 mmol, 2 eq) was added dropwise, followed by hydrogen peroxide (30%) (0.858 ml, 10.97 mmol, 3 eq). The resultant reaction mixture was stirred for 2 h at room temperature before being diluted with ice cold water and extracted with 10% methanol in DCM. The organic portion was washed with brine solution, dried over sodium sulfate, filtered and concentrated in vacuo to afford tert-butyl 5-(2-hydroxyethyl)-1-methyl-3,4-dihydroisoquinoline-2(1H)-carboxylate (0.600 g, 0.700 mmol, 19.1% yield) which was used without further purification. LCMS m/z=192.4 [M+H-Boc]⁺.

199: To a stirred solution of tert-butyl 5-(2-hydroxyethyl)-1-methyl-3,4-dihydroisoquinoline-2(1H)-carboxylate (310 mg, 1.063 mmol, 1 eq) in DCM (10 mL) was added DMP (676 mg, 1.595 mmol, 1.5 eq) portionwise at 0° C. The reaction mixture was stirred overnight at room temperature before being filtered and concentrated in vacuo to afford tert-butyl 1-methyl-5-(2-oxoethyl)-3,4-dihydroisoquinoline-2(1H)-carboxylate (320 mg, 1.105 mmol) which was used without further purification. LCMS m/z=190.4 [M+H-Boc]⁺.

200: By general procedure 1 using MP-CNBH₃ (w/w) in MeOH. Obtained 320 mg, 52% yield. LCMS m/z=671.4 [M+H]⁺.

201: By general procedure 2. Obtained 300 mg (crude). LCMS m/z=571.2 [M+H]⁺.

Compound B154: By general procedure 6 using (E/Z)-2-cyano-4,4-dimethylpent-2-enoic acid (1.3 eq).

Obtained 31 mg, 4.7% yield. LCMS m/z=706.4 [M+H]⁺.

### (E/Z)-3-(6-bromopyridin-2-yl)-2-(5-((4-(2,6-dimethoxy-4-(1,4,5-trimethyl-6-oxo-1,6-dihydropyridin-3-yl)benzoyl)piperazin-1-yl)methyl)-1-methyl-1,2,3,4-tetrahydroisoquinoline-2-carbonyl)acrylonitrile (B81)

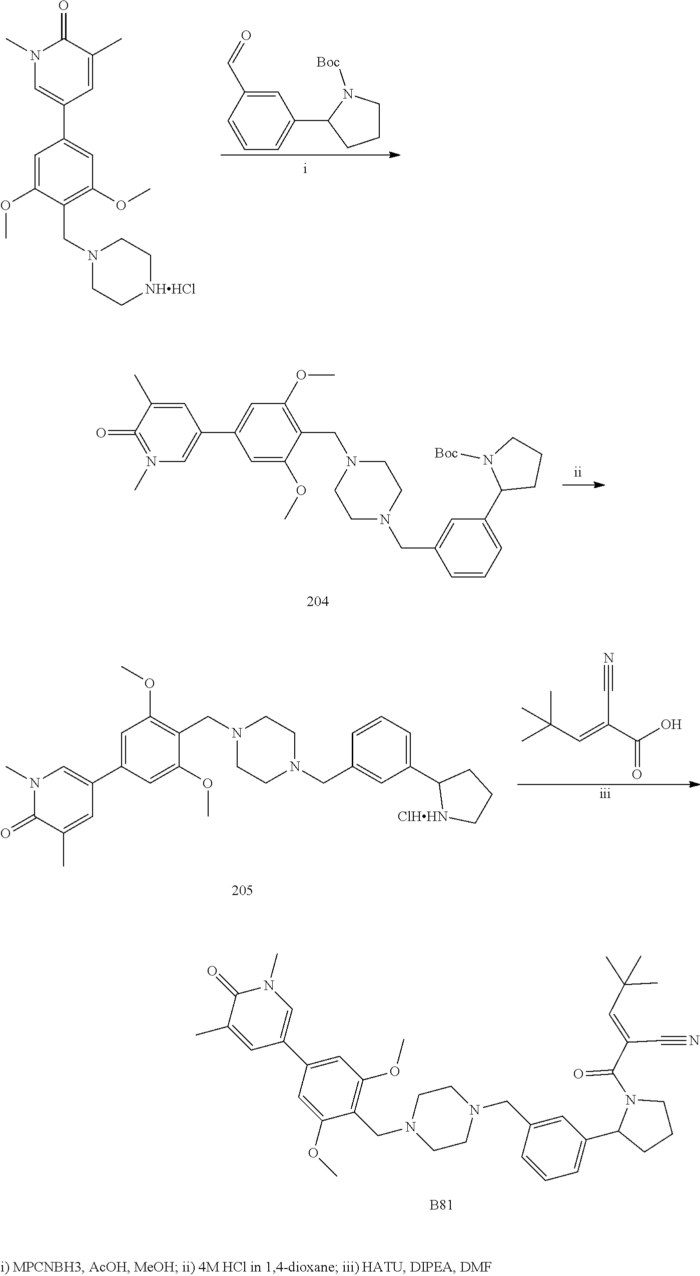

204: By general procedure 1 using tert-butyl 2-(3-formylphenyl) pyrrolidine-1-carboxylate (1.5 eq). Obtained 230 mg, 25.3% yield. LCMS m/z=617.3 [M+H]⁺.

205: By general procedure 2 using 4M HCl in 1,4-Dioxane (3 mL). Obtained 200 mg, 92% yield. LCMS m/z=517.3 [M+H]⁺.

Compound B81: By general procedure 6 using (E)-2-cyano-4,4-dimethylpent-2-enoic acid (2 eq). Obtained 33 mg, 15.8% yield. LCMS m/z=652.4 [M+H]⁺.

### (E/Z)-2-(5-((4-(2,6-dimethoxy-4-(1,4,5-trimethyl-6-oxo-1,6-dihydropyridin-3-yl)benzyl)-3,3-dimethyl-2-oxopiperazin-1-yl)methyl)-1-methyl-1,2,3,4-tetrahydroisoquinoline-2-carbonyl)-4,4-dimethylpent-2-enenitrile (B112)

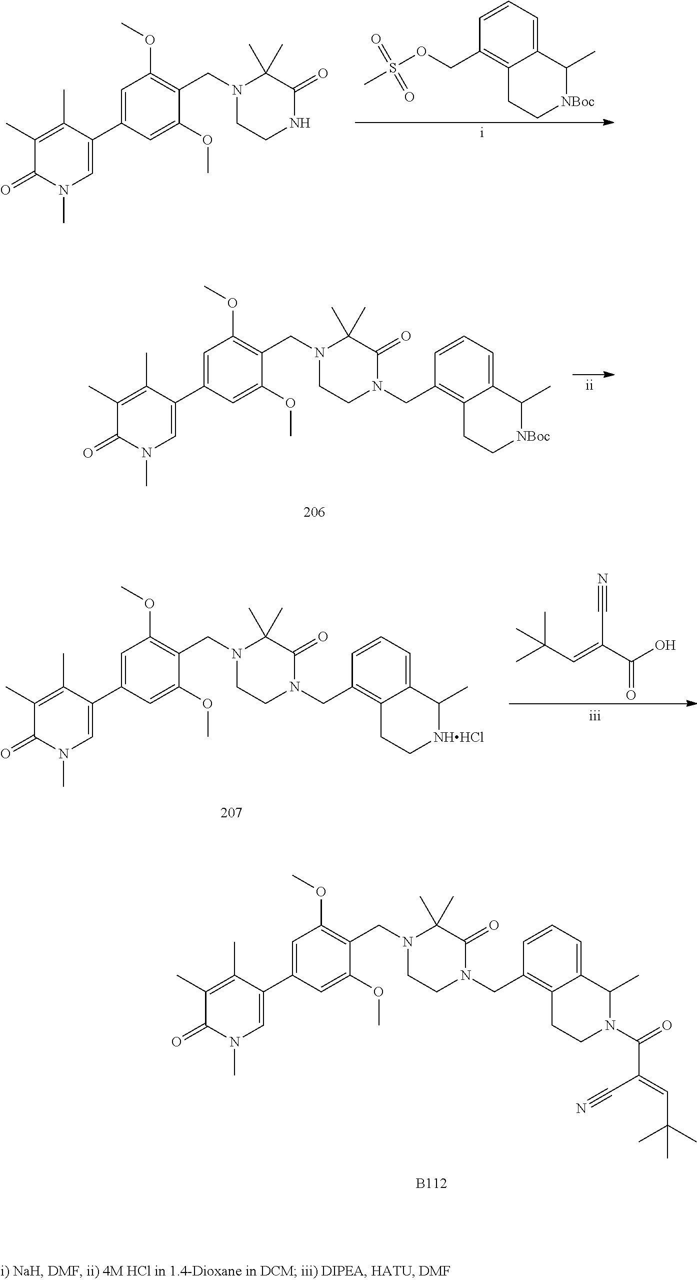

206: To a stirred solution of 4-(2,6-dimethoxy-4-(1,4,5-trimethyl-8-oxo-1,6-dihydropyridin-3-yl)benzyl)-3,3-dimethylpiperazin-2-one (0.17 g, 0.411 mmol, 1 eq) in DMF (2 mL) was added NaH (0.033 g, 0.822 mmol, 2 eq). After 5 min, tert-butyl 1-methyl-5-(((methylsulfonyl)oxy)methy)-3,4-dihydroisoquinoline-2(1H)-carboxylate (0.161 g, 0.452 mmol, 1.5 eq) was added to the reaction mixture at room temperature. The resultant reaction mixture was stirred at 50° C. overnight. The reaction mixture was quenched by adding ice cold water (50 mL) and extracted with DCM (3×40 mL). The organic layer was washed with excess of water, brine, dried over sodium sulfate and concentrated in vacuo. The obtained crude was purified by silica gel column chromatography (gradient=0-5% MeOH in DCM). The appropriate fractions were concentrated in vacuo to obtained tert-butyl 5-((4-(2,6-dimethoxy-4-(1,4,5-trimethyl-6-oxo-1,6-dihydropyridin-3-yl)benzyl)-3,3-dimethyl-2-oxopiperazin-1-yl)methyl)-1-methyl-3,4-dihydroisoquinoline-2(1H)-carboxylate (140 mg, 0.187 mmol, 45.5% yield) as off white solid. LCMS m/z=673.6 [M+H]⁺.

207: By general procedure 2. Obtained 100 mg (crude). LCMS m/z 573.3 [M+H]⁺.

Compound B112: By general procedure 6 using (E/Z)-2-cyano-4,4-dimethylpent-2-enoic acid (2 eq). Obtained (15 mg, 10% yield). LCMS m/z=708.4 [M+H]⁺.

### (E/Z)-3-(6-bromopyridin-2-yl)-2-(5-((1-(2,6-dimethoxy-4-(1,4,5-trimethyl-6-oxo-1,6-dihydropyridin-3-yl)benzoyl)piperidin-4-yl)oxy)-1-methyl-1,2,3,4-tetrahydroisoquinoline-2-carbonyl)acrylonitrile (B88)

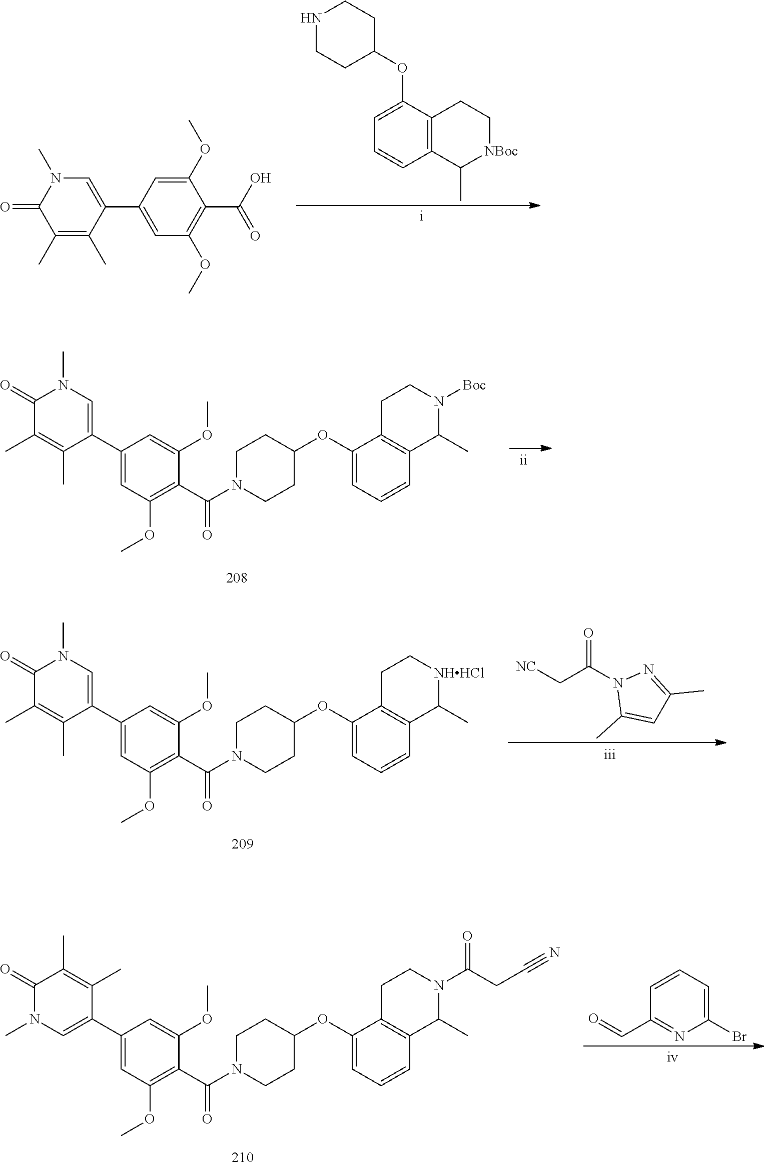

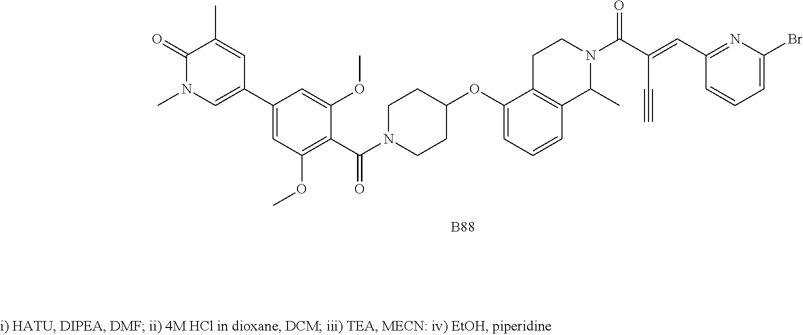

208: By general procedure 14 using MPCNBH₃ (w/w). Obtained 150 mg, 3.7% yield. LCMS m/z=646.3 [M+H]⁺

209: By general procedure 15 using 4M HCl in 1,4-Dioxane. Obtained 140 mg (crude), 96% yield. LCMS m/z=546.3 [M+H]⁺.

210: By general procedure 3b using 3-(3,5-dimethyl-1H-pyrazol-1-yl)-3-oxopropanenitrile (1.5 eq).

Obtained 28 mg, 22% yield. LCMS m/z=500.6 [M+H]⁺.

Compound B88: By general procedure 4a using 6-bromopicolinaldehyde (4 eq). Obtained 8 mg, 34.5% yield. LCMS m/z=782.2 [M+H]⁺.

### (E/Z)-3-(6-bromopyridin-2-yl)-2-(5-(1-(2,6-dimethoxy-4-(8-methyl-4-oxo-4,5-dihydrothieno[3,2-c]pyrindin-7-yl)benzyl)piperidin-4-yl)oxy)-1-methyl-1,2,3,4-tetrahydroisoquinoline-2-carbonyl)acrylonitrile (B77)

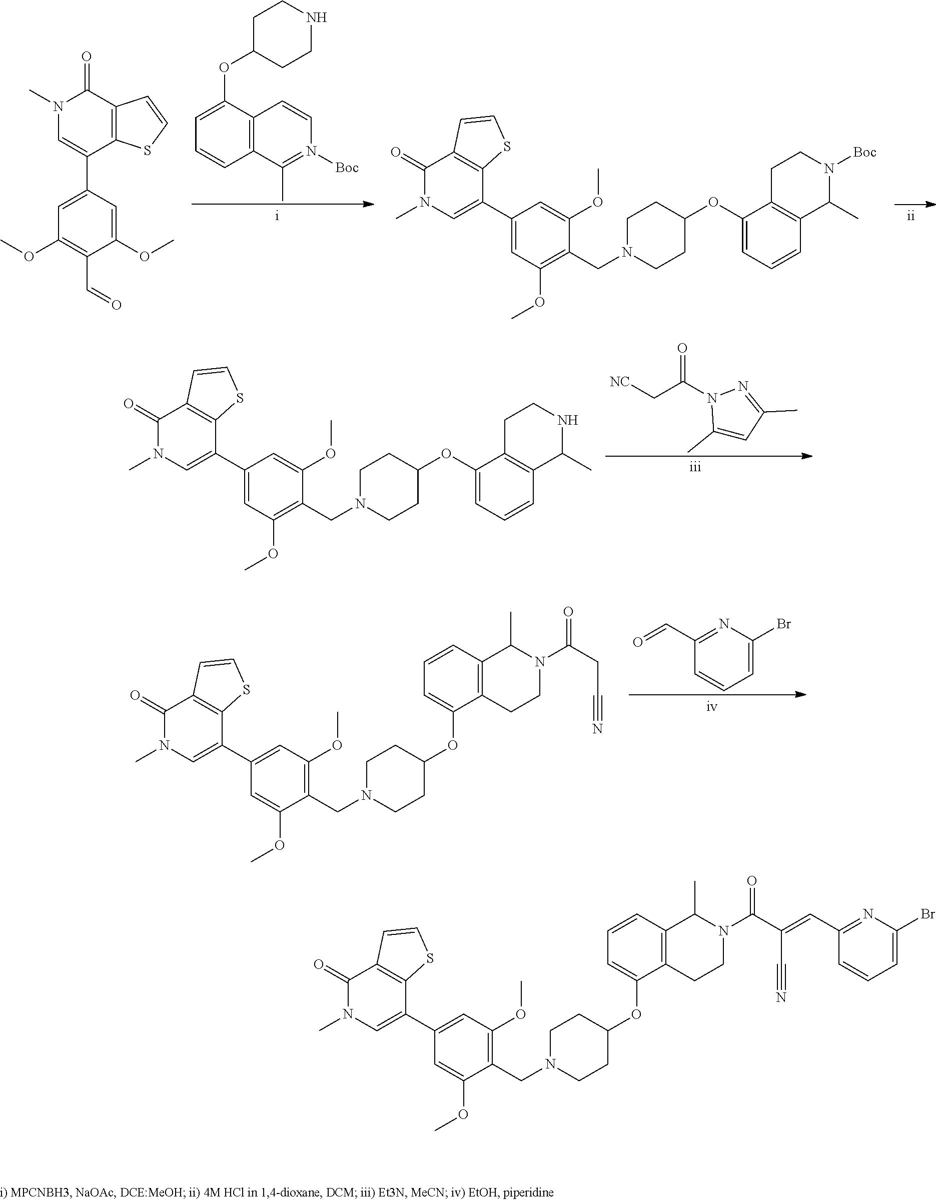

211: By general procedure 14a using MPCNBH₃ (w/w). in DCE:MeOH. Obtained 410 mg, 37.1% yield. LCMS m/z=680.2 [M+H]⁺.

212: By general procedure 2 using 4M HCl in Dioxane in DCM. Obtained 430 mg (crude), 98% yield. LCMS m/z=580.2 [M+H]⁺.

213: By general procedure 3b using 1-cyanoacetyl-3,5-dimethyl-1H-pyrazole (1.5 eq). Obtained 210 mg, 62% yield. LCMS m/z=687.2 [M+H]⁺.

Compound B77: By general procedure 4a using 6-bromopyridine-2-carboxaldehyde (3 eq) in ethanol. Obtained 31 mg, 23.0% yield. LCMS m/z=795.0 [M+H]⁺.

### 2-(2-(3-((3-(2,6-dimethoxy-4-(1,4,5-trimethyl-4-oxo-1,6-dihydropyridin-4-yl)benzyl)-3,6-diazabicyclo[3.1.1]heptan-6-yl)methyl)phenyl)pyrrolidine-1-carbonyl)-4,4-dimethylpentanenitrile (B6)

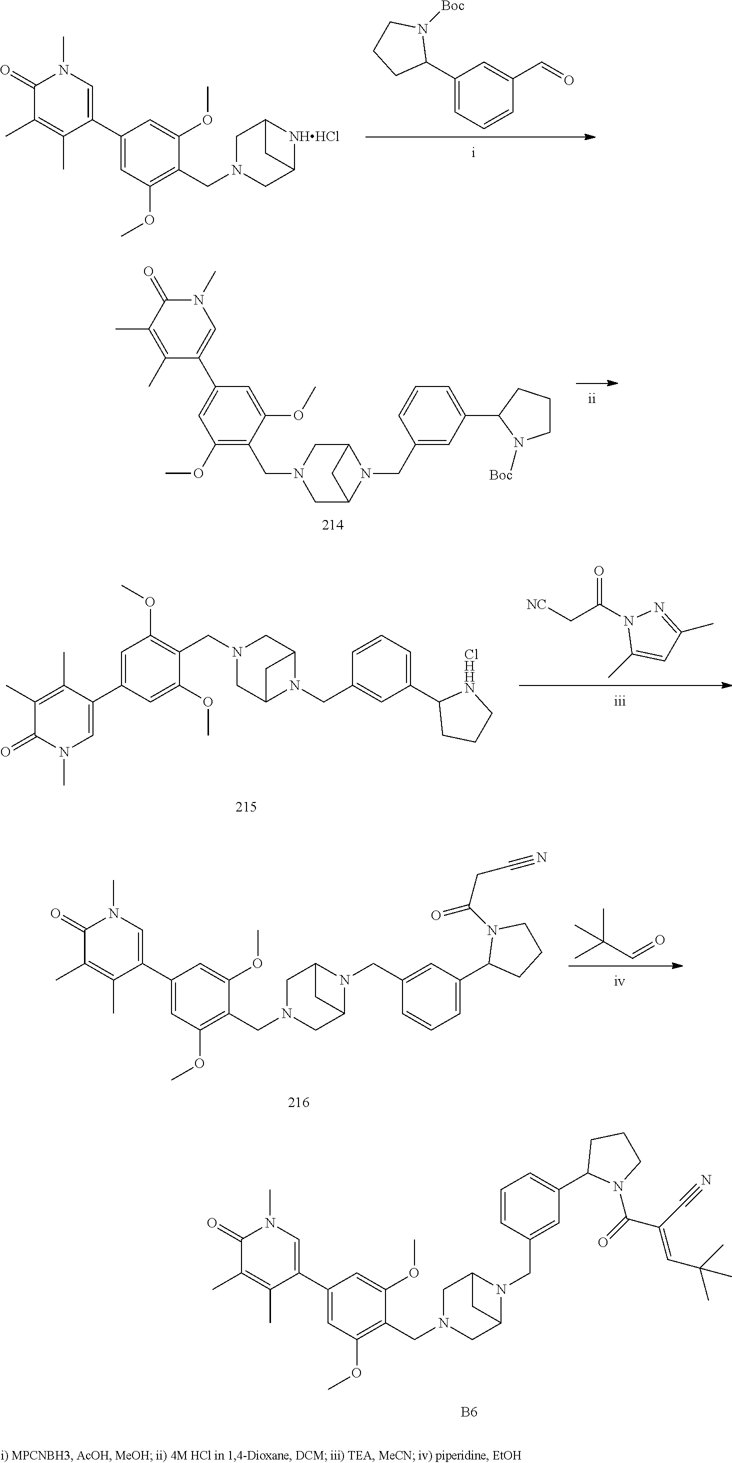

214: By general procedure 14a using MPCNBH₃ (w/w) in DCE:MeOH. Obtained 900 mg, 76% yield. LCMS m/z=643.4 [M+H]⁺.

215: By general procedure 2 using 4M HCl in dioxane in DCM. Obtained 0.7 g, 92% yield. LCMS m/z=543.3 [M+H]⁺.

216: By general procedure 3b using 3-(3,5-dimethyl-1H-pyrazol-1-yl)-3-oxopropanenitrile (1.2 eq). Obtained 450 mg, 61.1% yield. LCMS m/z=610.3 [M+H]⁺.

Compound B6 By general procedure 17 using pivalaldehyde (3.0 eq) in ethanol. Obtained 20 mg, 12.0% yield. LCMS m/z=679.4 [M+H]⁺.

### Compound B12: (E/Z)-3-(6-bromopyridin-2-yl)-2-(2-(3-((3-(2,6-dimethoxy-4-(1,4,5-trimethyl-6-oxo-1,6-dihydropyridin-3-yl)benzyl)-3,6-diazabicyclo[3.1.1]heptan-6-yl)methyl)phenyl)pyrrolidine-1-carbonyl)acrylonitrile

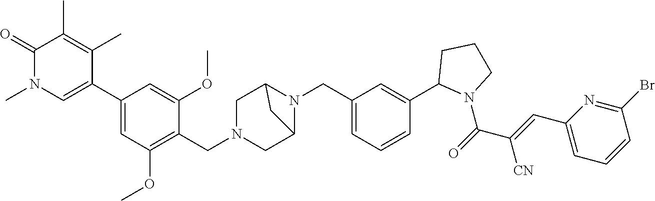 [SMILES: COc1cc(-c2cn(C)c(=O)c(C)c2C)cc(OC)c1CN1CC2CC(C1)N2Cc1cccc(C2CCCN2C(=O)/C(C#N)=C/c2cccc(Br)n2)c1]

Prepared by following general procedure 4a using 215 and 6-bromopicolinaldehyde (3.0 eq) in ethanol. Obtained 8 mg, 23.7% yield. LCMS m/z=777.2 [M+H]⁺.

### (E/Z)-2-(2-(4-(((2,6-dimethoxy-4-(1,4,5-trimethyl-6-oxo-1,6-dihydropyridin-3-yl)benzyl)methyl)amino)methyl)phenyl)pyrrolidine-1-carbonyl)-4,4-dimethylpent-2-enenitrile (B34)

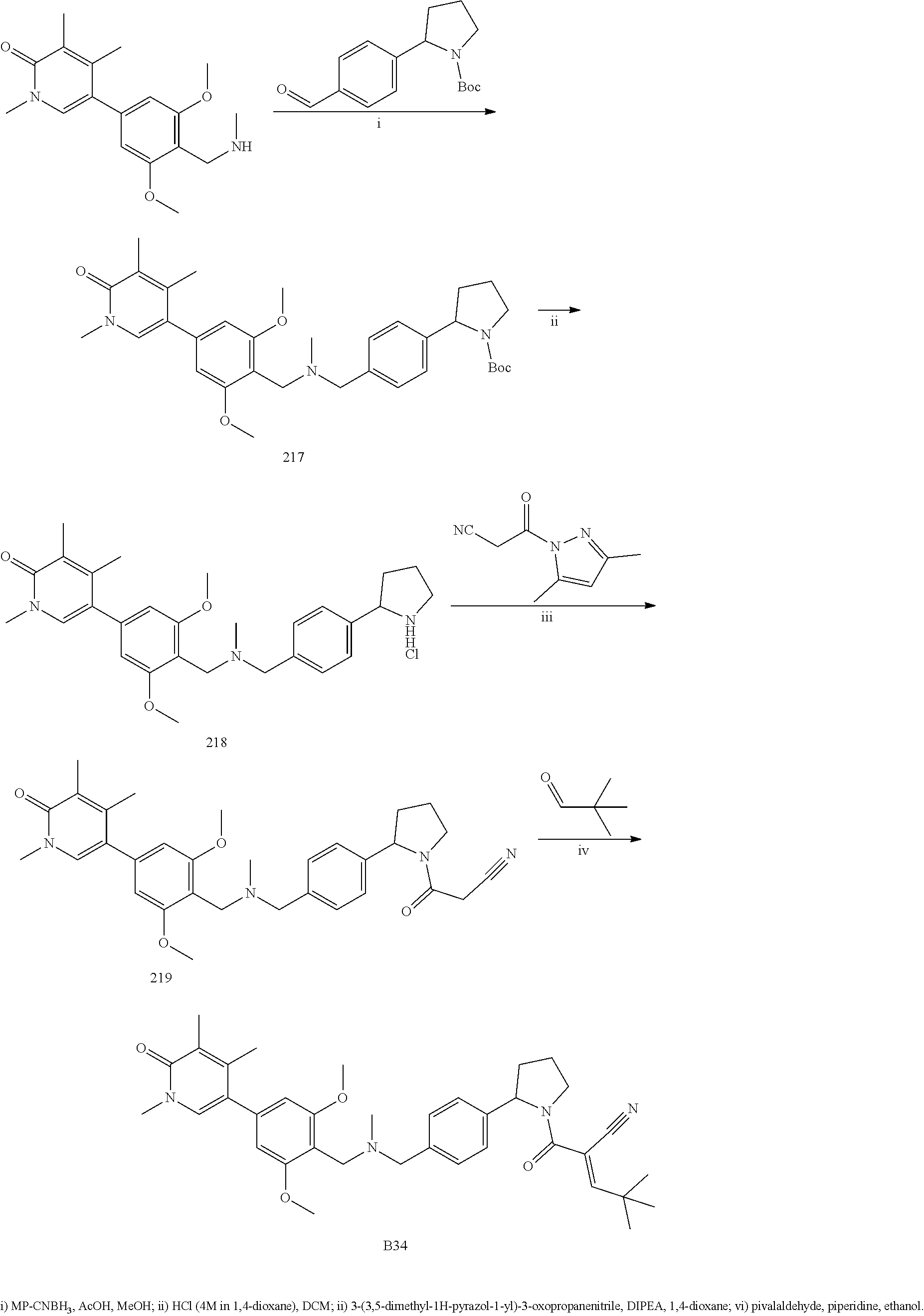

217: By general procedure 14 using MP-CNBH₃ (w/w) in MeOH. Obtained 1 g, 83% yield. LCMS m/z=576.4 [M+H]⁺.

218: By general procedure 2. Obtained 0.9 g (crude). LCMS m/z=476.3 [M+H]⁺.

220: By general procedure 3b. Obtained 350 mg, 31% yield. LCMS m/z=543.3 [M+H]⁺.

Compound B34: By general procedure 4a using pivalaldehyde (3 eq) in EtOH. Obtained 25 mg, 14.5% yield. LCMS m/z=611.2 [M+H]⁺.

### (E/Z)-2-(2-(3-(4-(2,6-dimethoxy-4-(1,4,5-trimethyl-6-oxo-1,6-dihydropyridin-3-yl)benzyl)piperazine-1-carbonyl)phenyl)pyrrolidine-1-carbonyl)-4,4-dimethylpent-2-enenitrile (B35)

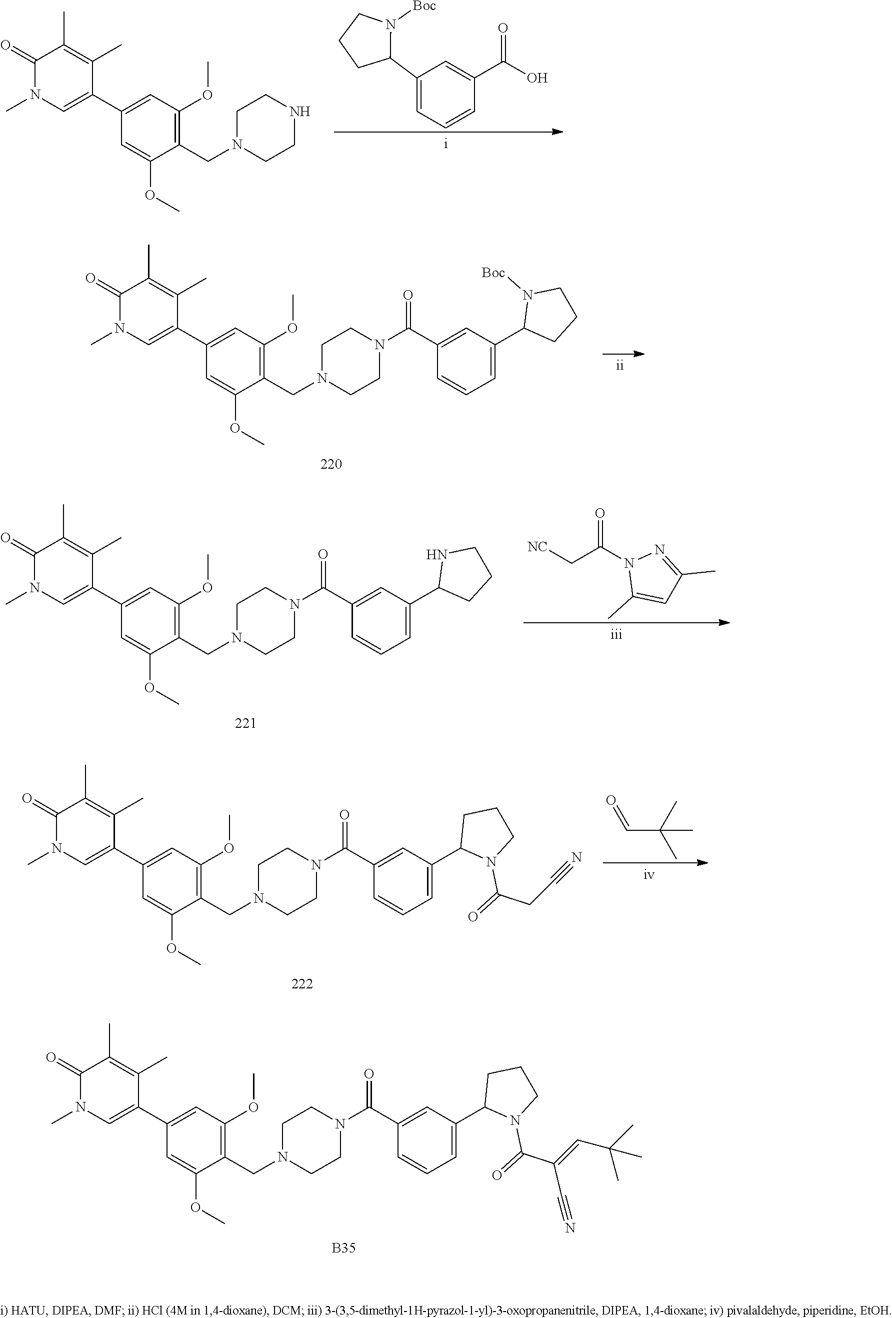

220: By general procedure 6. Obtained 475 mg, 25% yield. LCMS m/z=645.4 [M+H]⁺.

221: By general procedure 2 in 4M HCl in 1,4-dioxane in DCM. Obtained 485 mg crude. LCMS m/z=545.4 [M+H]⁺.

222: By general procedure 3b using 3-(3,5-dimethyl-1H-pyrazol-1-yl)-3-oxopropanenitrile (1.3 eq) in MeCN. Obtained 300 mg, 52.9% yield. LCMS m/z=612.6 [M+H]⁺.

B35: By general procedure 4a using pivalaldehyde (4 eq) in ethanol. Obtained 8 mg, 5% yield. LCMS m/z=680.2 [M+H]

### (E/Z)-2-(5-(6-(2,6-dimethoxy-4-(1,4,5-trimethyl-6-oxo-1,6-dihydropyridin-3-yl)benzyl)-2,6-diazaspiro[3.4]octan-2-yl)-1-methyl-1,2,3,4-tetrahydroisoquinoline-2-carbonyl)-4,4-dimethylpent-2-enenitrile (B40)

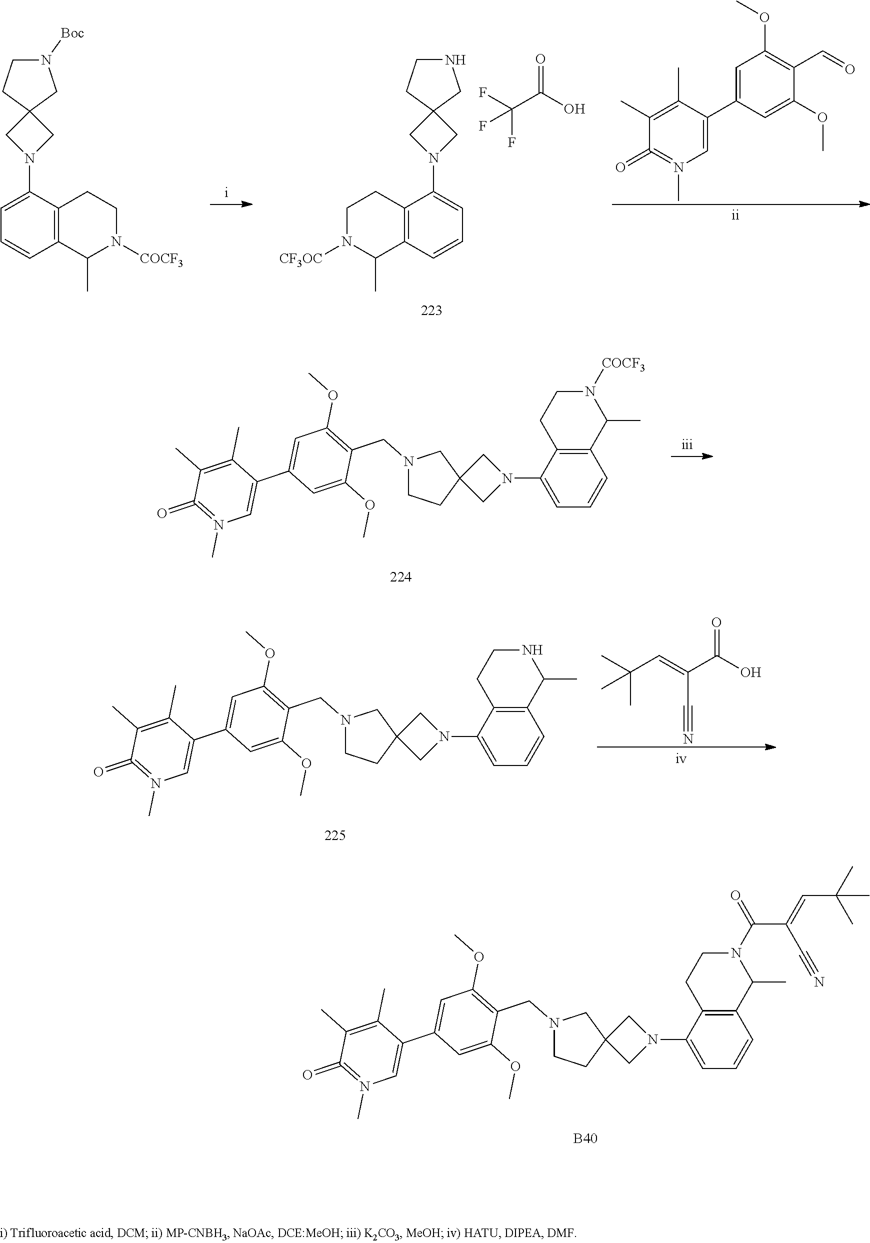

223: By general procedure 2. Obtained 1.2 g, Crude. LCMS m/z=354.2

224: By general procedure 14 using MP-CNBH₃ (w/w) in DCE:MeOH. Obtained 2 g, 85% yield. LCMS m/z=639.6 [M+H]⁺.

225: By general procedure 20 using K2CO3 (4 eq) in water:MeOH (1:4). Obtained 1.4 g (crude), 79% yield. LCMS m/z=545.3 [M+H]⁺.

B40: By general procedure 6 using (E/Z)-2-cyano-4,4-dimethylpent-2-enoic acid (2 eq) in DMF. Obtained 42 mg, 16% yield. LCMS m/z=678.3 [M+H]⁺.

### (E/Z)-2-(5-(9-(2,6-dimethoxy-4-(1,4,5-trimethyl-6-oxo-1,6-dihydropyridin-3-yl)benzyl)-3,9-diazaspiro[5.5]undecan-3-yl)-1-methyl-1,2,3,4-tetrahydroisoquinoline-2-carbonyl)-4,4-dimethylpent-2-enenitrile (B39)

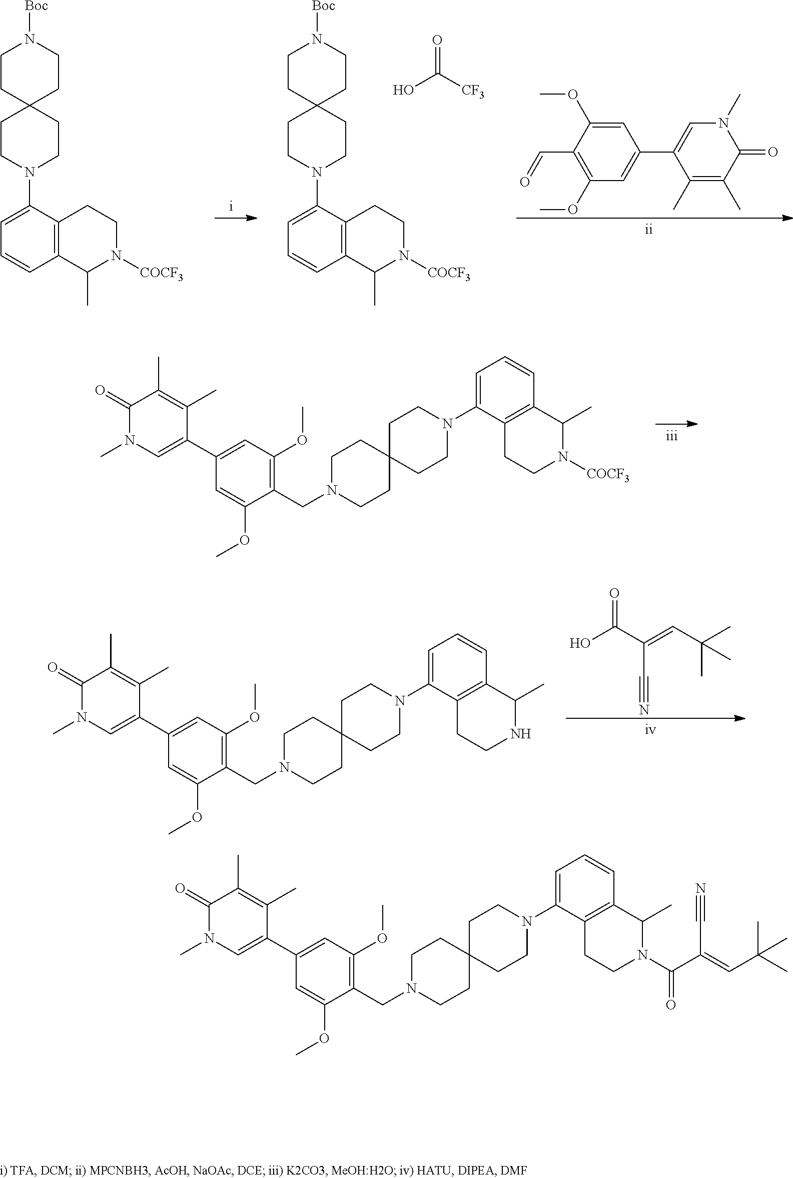

226: By general procedure 2 using TFA (0.5 mL) Obtained 1.2 g, 98% yield. LCMS m/z=396.2 [M+H]⁺.

227: By general procedure 14 using MPCNBH₃ (w/w). Obtained 650 mg, 36.6% yield. LCMS m/z=681.3 [M+H]⁺.

228: By general procedure 20. Obtained 520 mg, 81% yield. LCMS m/z=585.4 [M+H]⁺.

B39: By general procedure 6 using (E/Z)-2-cyano-4,4-dimethylpent-2-enoic acid Obtained 30 mg, 11.9% yield. LCMS m/z=720.4 [M+H]⁺.

### (E/Z)-2-(2-(3-(4-(2,6-dimethoxy-4-(1,4,5-trimethyl-4-oxo-1,6-dihydropyridin-3-yl)benzyl)piperazin-1-yl)phenyl)pyrrolidine-1carbonyl)-4,4-dimethylpent-2-enenitrile (1B41)

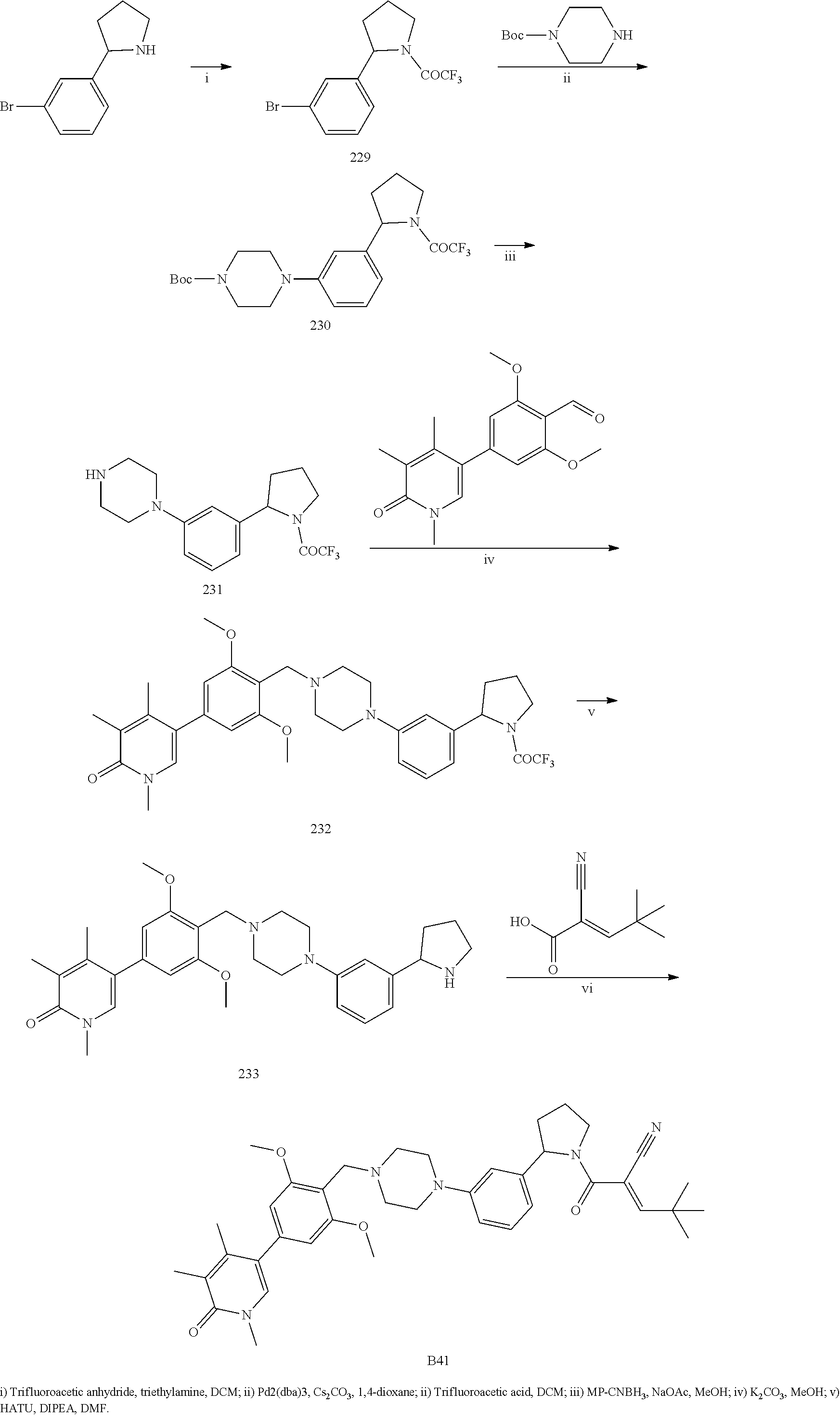

229: By general procedure 22. Obtained 800 mg, 55.6% yield. LCMS m/z=322.0 [M+H]⁺.

230: By general procedure 21a. Obtained 250 mg, 28% yield. LCMS m/z=428.2 [M+H]⁺.

231: By general procedure 2. Obtained 500 mg, Crude. LCMS m/z=328.2 [M+H]⁺.

232: By general procedure 14 using MP-CNBH₃ (w/w) in DCE:MeOH. Obtained 600 mg, 44.8% yield. LCMS m/z=613.2 [M+H]⁺.

233: By general procedure 20. Obtained 400 mg (crude). LCMS m/z=517.2 [M+H]⁺.

Compound B41: By general procedure 6 using (E/Z)-2-cyano-4,4-dimethylpent-2-enoic acid (2 eq) in DMF. Obtained 43 mg, 28% yield. LCMS m/z=652.3 [M+H]⁺.

### Compound B106: (E/Z)-2-(2-(3-(4-(2,6-dimethoxy-4-(1,4,5-trimethyl-6-oxo-1,6-dihydropyridin-3-yl)benzyl)piperazin-1-yl)phenyl)pyrrolidine-1-carbonyl)-4-methylpent-2-enenitrile

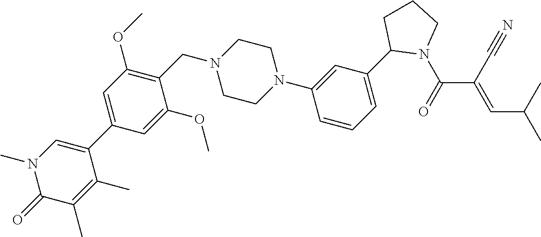 [SMILES: COc1cc(-c2cn(C)c(=O)c(C)c2C)cc(OC)c1CN1CCN(c2cccc(C3CCCN3C(=O)/C(C#N)=C/C(C)C)c2)CC1]

Prepared following general procedure 4 from 233. Obtained 53 mg, 32% yield. LCMS m/z=638.4 [M+H]⁺.

### (E/Z)-2-(2-(4-(4-(2,6-dimethoxy-4-(1,4,5-trimethyl-6-oxo-1,6-dihydropyridin-3-yl)benzyl)piperazin-1-yl)phenyl)pyrrolidine-1-carbonyl)-4,4-dimethylpent-2-enenitrile (B42)

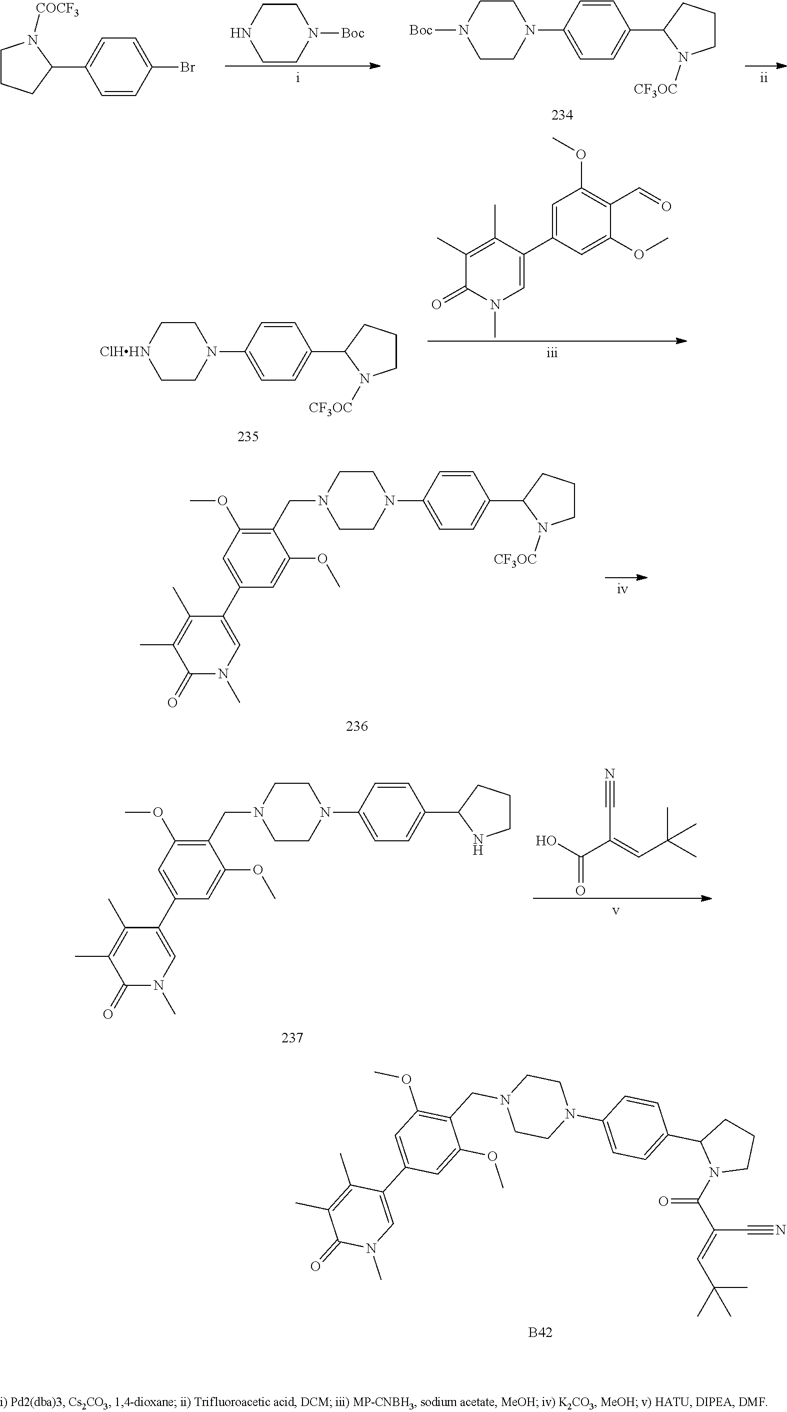

234: Prepared by following procedure 23. Obtained 530 mg, 69% yield. LCMS m/z=428.2 [M+H]⁺.

235: By general procedure 2. Obtained 450 mg, Crude. LCMS m/z=328.2 [M+H]⁺.

236: By general procedure 14. Obtained 420 mg, 51% yield. LCMS m/z=613.2 [M+H]⁺.

237: By general procedure 20. Obtained 285 mg (crude). LCMS m/z=517.2 [M+H]⁺.

Compound B42: By general procedure 6 using (E/Z)-2-cyano-4,4-dimethylpent-2-enoic acid (2 eq). Obtained 86 mg, 45% yield. LCMS m/z=652.4 [M+H]

### (E)-2-(5-(((S)-1-(2,6-dimethoxy-4-(1,4,5-trimethyl-6-oxo-1,6-dihydropyridin-3-yl)benzyl)pyrrolidin-3-yl)oxy)-1-methyl-1,2,3,4-tetrahydroisoquinoline-2-carbonyl)-4,4-dimethylpent-2-enenitrile (B134)

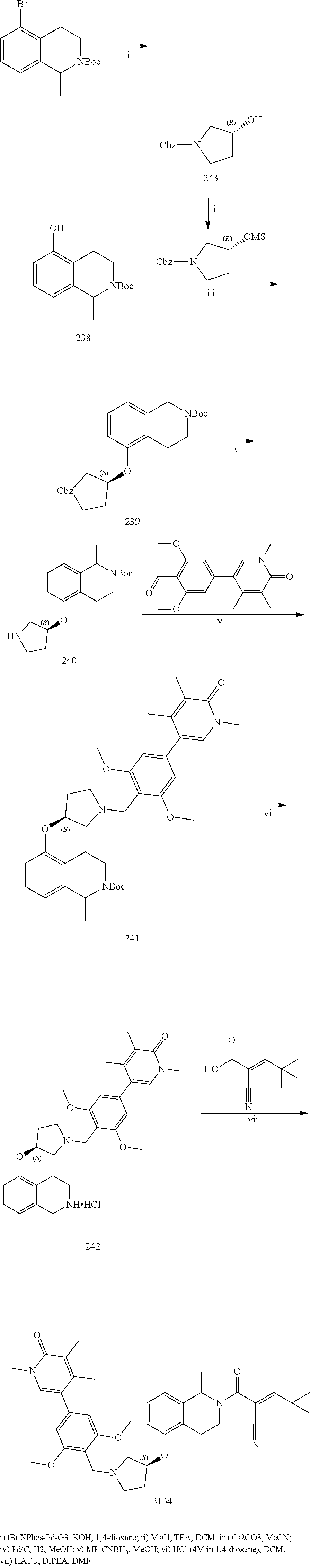

238: By general procedure 24. Obtained 3.8 g, 94% yield. LCMS m/z=208.2 [M−56]⁺.

243: By general procedure 25. Obtained 650 mg, 73.5% yield. LCMS m/z=300.0 [M+H]⁺.

239: By general procedure 26. Obtained 550 mg, 88% yield. LCMS m/z=367 [M-Boc]⁺.

240: By general procedure 27. Obtained 400 mg, 89% yield. LCMS m/z=333.4 [M+H]⁺.

241: By general procedure 1 using MP-CNBH₃ (1 equiv.) in MeOH. Obtained 400 mg, 75% yield. LCMS m/z=618.4 [M+H]⁺.

242: By general procedure 2 using 4M HCl in 1,4-dioxane in DCM. Obtained 350 mg (crude), 94% yield. LCMS m/z=518.3 [M+H]⁺.

B134: By general procedure 6 using (E/Z)-2-cyano-4,4-dimethylpent-2-enoic acid (2 equiv) in DMF. Obtained 35 mg, 0.053 mmol, 19% yield. LCMS m/z=653.4 [M+H]⁺

### (E/Z)-2-(5-((4-(2-methoxy-4-(1,4,5-trimethyl-6-oxo-1,6-dihydropyridin-3-yl)phenoxy)piperidin-1-yl)methyl)-1-methyl-1,2,3,4-tetrahydroisoquinoline-2-carbonyl)-4,4-dimethylpent-2-enenitrile (B127)

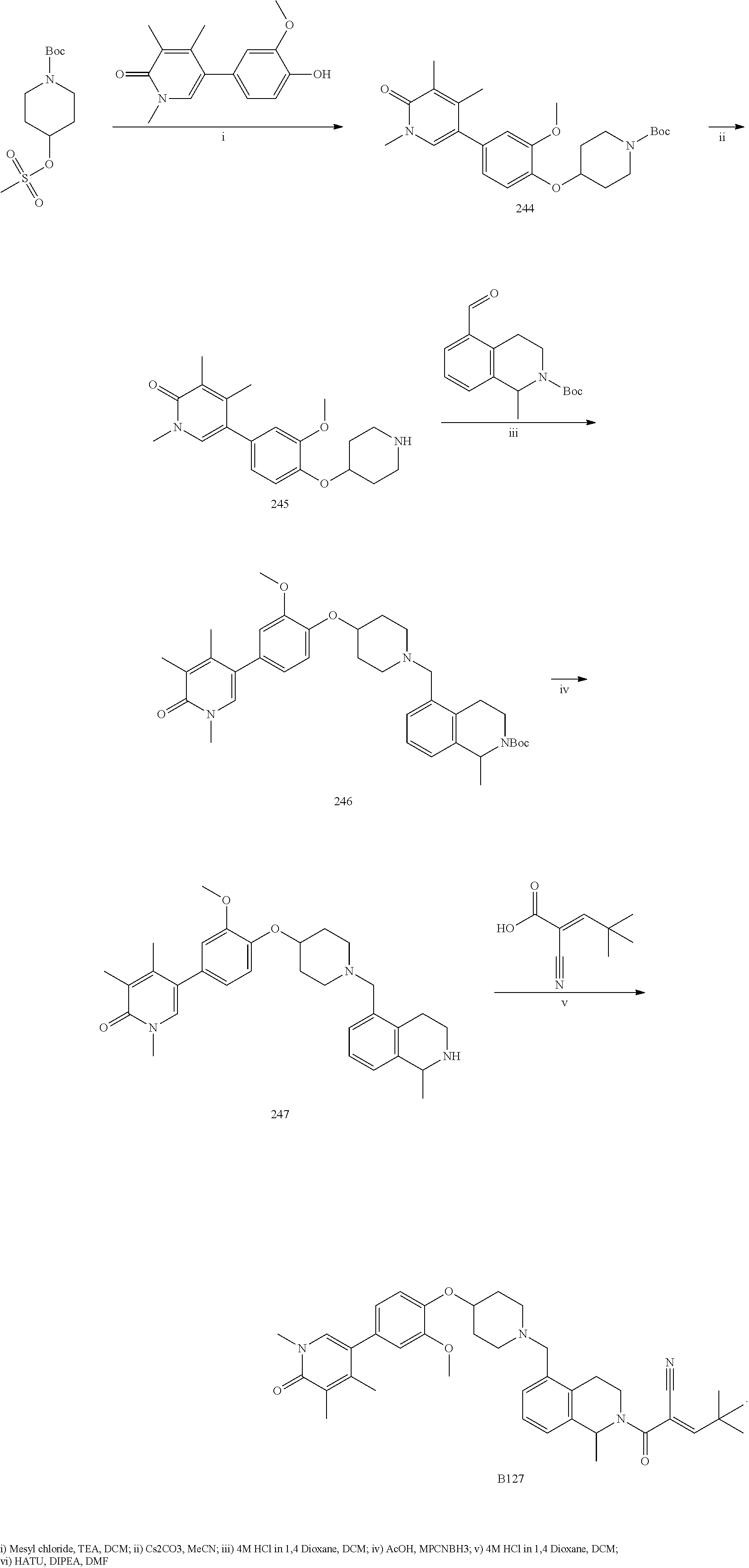

244: By general procedure 26 using starting material prepared in WO2008/42282. Obtained 400 mg, 57% yield. LCMS m/z=443.2 [M+H]⁺.

245: By general procedure 2 using 4M HCl in 1,4-dioxane. Obtained 500 mg (crude). LCMS m/z=343.2 [M+H]⁺.

246: By general procedure 1 using MPCNBH₃ (w/w). Obtained 230 mg, 26% yield. LCMS m/z=602.3 [M+H]⁺.

247: By general procedure 2 using 4M HCl in 1,4-dioxane. Obtained 230 mg (crude). LCMS m/z=502.2 [M+H]⁺.

Compound B127: By general procedure 6 using (E/Z)-2-cyano-4,4-dimethylpent-2-enoic acid (2 eq). Obtained 52 mg, 16% yield. LCMS m/z=637.4 [M+H]⁺.

### Synthesis of Single Enantiomer of (E/Z)-2-(5-((4-(2-methoxy-4-(1,4,5-trimethyl-6-oxo-1,6-dihydropyridin-3-yl)phenoxy)piperidin-1-yl)methyl)-1-methyl-1,2,3,4-tetrahydroisoquinoline-2-carbonyl)-4,4-dimethylpent-2-enenitrile (C31)

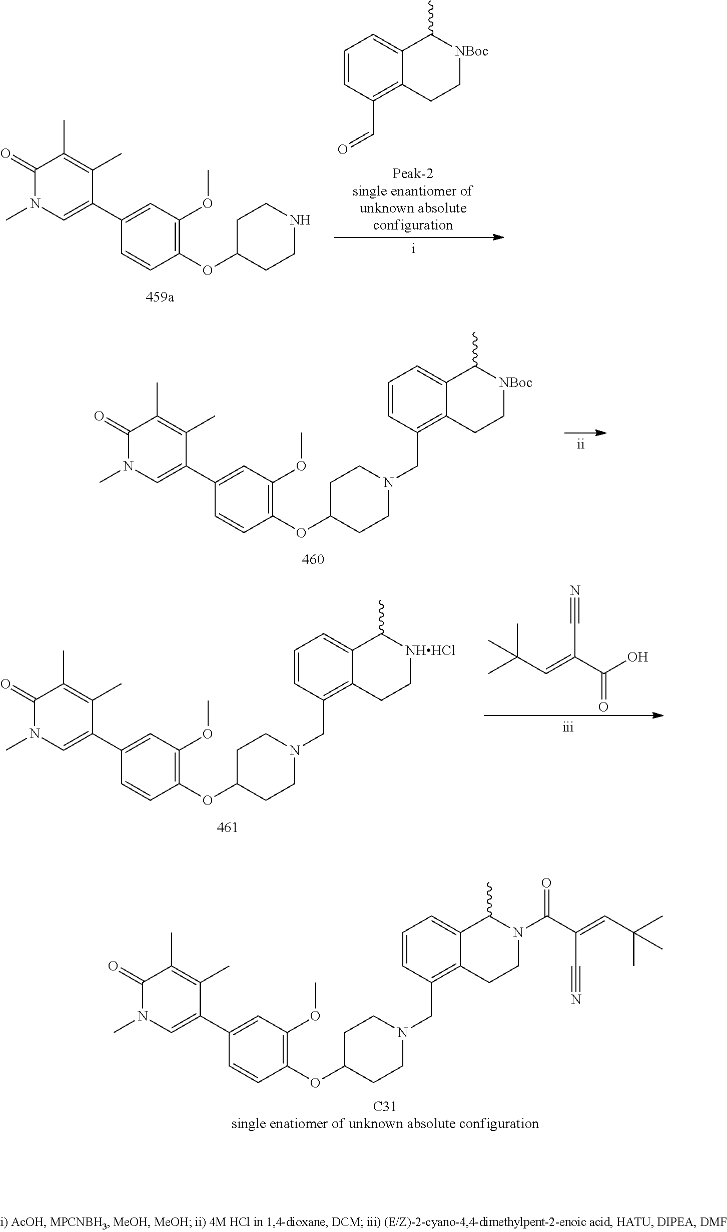

459a: Prepared via 1-step cross-coupling reaction of 5-bromo-1,3,4-trimethylpyridin-2-one (as prepared in WO2021/178920) with 4-hydroxy-3-methoxyphenylboronic acid pinacol ester (commercial) using XPhosPdG2, K3PO4, THF:H2O, 80° C.;

460: By general procedure 1 using MPCNBH₃ (w/w) in MeOH. Obtained 110 ng, 27.9% yield. LCMS m/z=602.2 [M+H]⁺.

461: By general procedure 2 using dioxane in HCl. Obtained 80 mg, 95% yield. LCMS m/z=502.2 [M+H]⁺.

Compound C31: By general procedure 6 using (E/Z)-2-cyano-4,4-dimethylpent-2-enoic acid (1.2 eq) in DMF. Obtained 44 mg, 31% yield. LCMS m/z=637.3 [M+H]⁺

### C32: (E/Z)-2-(1-isopropyl-5-((4-(2-methoxy-4-(1,4,5-trimethyl-6-oxo-1,6-dihydropyridin-3-yl)phenoxy)piperidin-1-yl)methyl)-1,2,3,4-tetrahydroisoquinoline-2-carbonyl)-4,4-dimethylpent-2-enenitrile

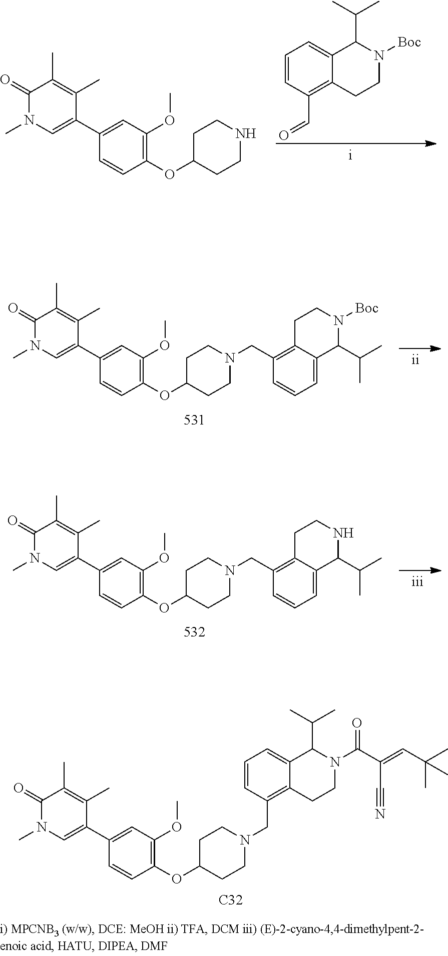 [SMILES: COc1cc(-c2cn(C)c(=O)c(C)c2C)ccc1OC1CCNCC1 ; CC(C)C1c2cccc(C=O)c2CCN1C(=O)OC(C)(C)C ; COc1cc(-c2cn(C)c(=O)c(C)c2C)ccc1OC1CCN(Cc2cccc3c2CCN(C(=O)OC(C)(C)C)C3C(C)C)CC1 ; COc1cc(-c2cn(C)c(=O)c(C)c2C)ccc1OC1CCN(Cc2cccc3c2CCNC3C(C)C)CC1 ; COc1cc(-c2cn(C)c(=O)c(C)c2C)ccc1OC1CCN(Cc2cccc3c2CCN(C(=O)/C(C#N)=C/C(C)(C)C)C3C(C)C)CC1 ; "C32"]

531: By general procedure 1a using MP-CNBH₃ (w/w) in MeOH:DCE (0.4:2). Obtained 400 mg, 95% yield. LCMS m/z=630.4 [M+H]⁺.

532: By general procedure 2b using TFA (7 eq) in DCM. Obtained 300 mg, crude. LCMS m/z=530.4 [M+H]⁺

Compound C32: By general procedure 6. Obtained 16 mg, 11% yield. LCMS m/z=665.4 [M+H]⁺.

### (E/Z)-2-(5-(((S)-4-(2,6-dimethoxy-4-(1,4,5-trimethyl-6-oxo-1,6-dihydropyridin-3-yl)benzyl)-2-ethylpiperazin-1-yl)methyl)-1-methyl-1,2,3,4-tetrahydroisoquinoline-2-carbonyl)-4,4-dimethylpent-2-enenitrile (B142)

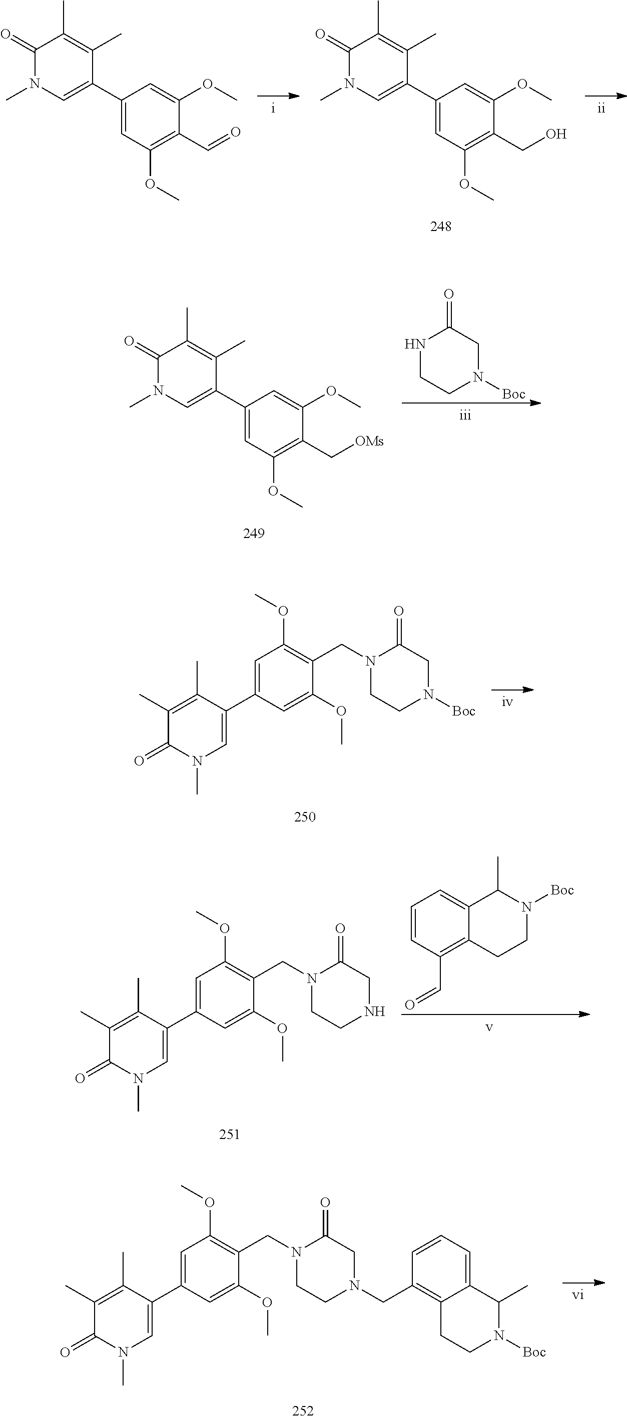

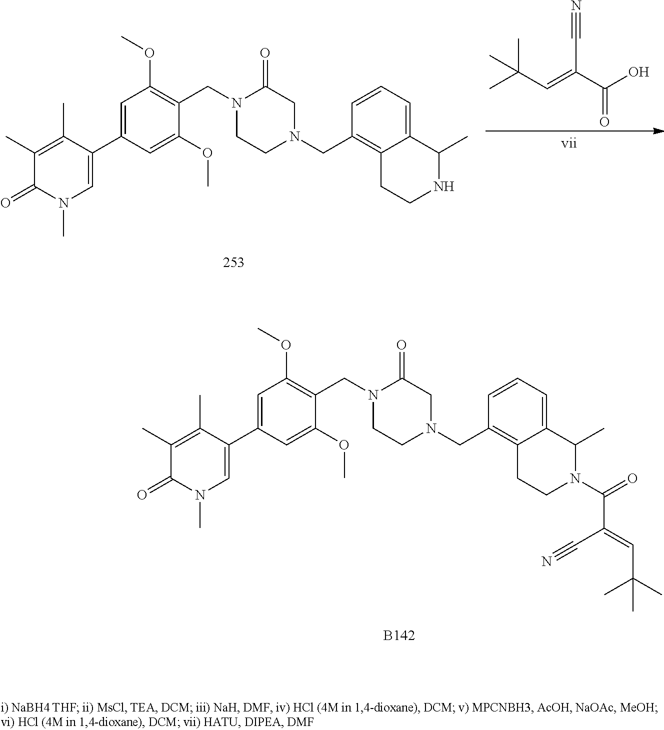

248: To a stirred solution of 2,6-dimethoxy-4-(1,4,5-trimethyl-6-oxo-1,6-dihydropyridin-3-yl)benzaldehyde (500 mg, 1.659 mmol, 1 eq) in THF (5 mL) at 0° C. was added NaBH4 (94 mg, 2.489 mmol, 1.5 eq) and the reaction was stirred at RT for 2 h. The reaction was quenched with ice-cold water and extracted with DCM. The organic layer was concentrated in vacuo and the crude compound was purified by silica gel column chromatography (gradient=2% MeOH/DCM). The appropriate fractions were concentrated in vacuo to afford 5-(4-(hydroxymethyl)-3,5-dimethoxyphenyl)-1,3,4-trimethylpyridin-2(1H)-one (400 mg, 1.239 mmol, 74.7% yield). LCMS m/z=304.4 [M+H]⁺.

249: By general procedure 25. Obtained 320 mg, 28% yield.

250: To a stirred solution of tert-butyl 3-oxopiperazine-1-carboxylate (315 mg, 1.573 mmol, 2 eq) in DMF (5 ml) at 0° C. was added NaH (62.9 mg, 1.573 mmol, 2 eq). After 5 min, 2,6-dimethoxy-4-(1,4,5-trimethyl-6-oxo-1,6-dihydropyridin-3-yl)benzyl methanesulfonate (300 mg, 0.786 mmol, 1 eq) was added. The reaction was stirred at 50° C. for 16 h before being purified directly by silica gel column chromatography (gradient=2% MeOH/DCM). The appropriate fractions were concentrated in vacuo to afford tert-butyl 4-(2,6-dimethoxy-4-(1,4,5-trimethyl-6-oxo-1,6-dihydropyridin-3-yl)benzyl)-3-oxopiperazine-1-carboxylate (120 mg, 0.245 mmol, 31.1% yield). LCMS m/z=486.2 [M+H]⁺.

251: By general procedure 2. Obtained 80 mg (crude). LCMS m/z=386.3 [M+H]⁺.

252: By general procedure 14b using MP-CNBH₃ (w/w) in MeOH. Obtained 80 mg, 46% yield. LCMS m/z=654.4 [M+H]⁺.

253: By general procedure 2. Obtained 68 mg (crude). LCMS m/z=545.3 [M+H]⁺.

Compound B142: By general procedure 6 using (E/Z)-2-cyano-4,4-dimethylpent-2-enoic acid (1.5 eq) in DMF. Obtained 20 mg, 18% yield. LCMS m/z=680.4 [M+H]⁺.

### (E/Z)-3-(6-bromopyridin-2-yl)-2-(5-((1-(2,6-dimethoxy-4-(1,4,5-trimethyl-6-oxo-1,6-dihydropyridin-3-yl)benzyl)piperidin-4-yl)methoxy)-1-methyl-1,2,3,4-tetrahydroisoquinoline-2-carbonyl)acrylonitrile (B95)

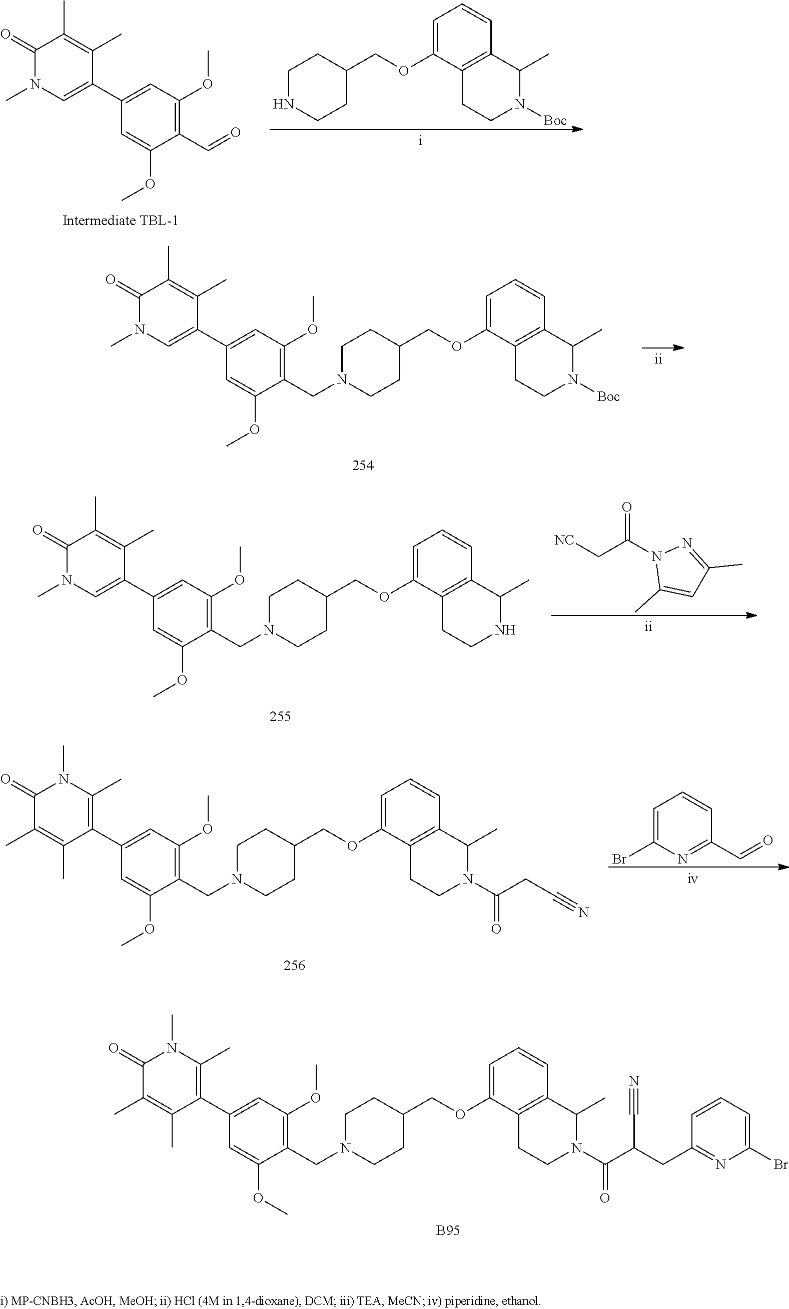

254: By general procedure 1 using MP-CNBH₃ (1 equiv.) in MeOH. Obtained 400 mg, 59.1% yield. LCMS m/z=646.4 [M+H]⁺.

255: By general procedure 2. Obtained 400 mg (crude). LCMS m/z=548.2 [M+H]⁺.

256: By general procedure 3 using 3-(3,5-dimethyl-1H-pyrazol-1-yl)-3-oxopropanenitrile (3 eq) in MeCN. Obtained 400 mg, 29% yield. LCMS m/z=613.3 [M+H]⁺.

B95: By general procedure 4 in EtOH. Obtained 50 mg, 43% yield. LCMS m/z=782.2 [M+H]⁺.

### tert-butyl 5-((1-((benzyloxy)carbonyl)piperidin-4-yl)methoxy)-1-methyl-3,4-dihydroisoquinoline-2(1H)-carboxylate

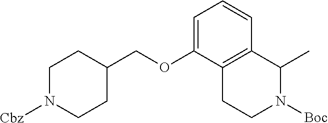 [SMILES: CC1c2cccc(OCC3CCN(C(=O)OCc4ccccc4)CC3)c2CCN1C(=O)OC(C)(C)C]

By general procedure 26 using starting material synthesised according to WO2011/31628. Obtained 520 mg, 99% yield. LCMS m/z=395.2 [M+H]⁺.

### (E/Z)-2-(5-((7-(2-methoxy-4-(1,4,5-trimethyl-6-oxo-1,6-dihydropyridin-3-yl)benzyl)-4,7-diazaspiro[2.5]octan-4-yl)methyl)-1-methyl-1,2,3,4-tetrahydroisoquinoline-2-carbonyl)-4,4-dimethylpent-2-enenitrile (B147)

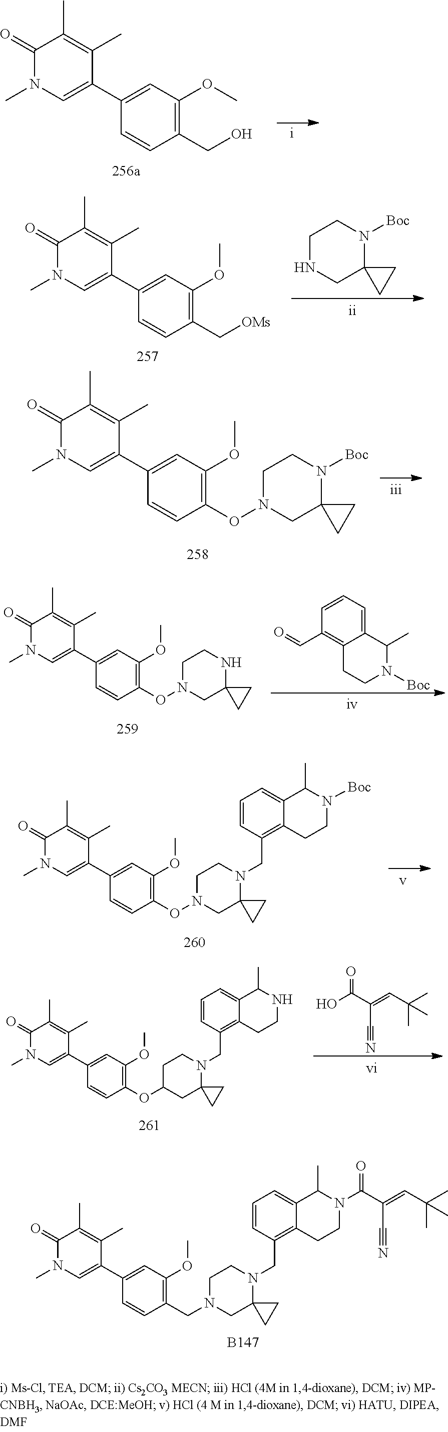

256a: Prepared via 1-step cross-coupling reaction of 5-bromo-1,3,4-trimethylpyridin-2-one (as prepared in WO2021/178920) with (2-methoxy-4-(4,4,5,5-tetramethyl-1,3,2-dioxaborolan-2-yl)phenyl)methanol (commercial) using XPhosPdG3, K3PO4, THF:H2O, 80° C.;

257: By general procedure 25 using. Obtained 250 mg, 97% yield.

258: By general procedure 26. Obtained 225 mg, 96% yield. LCMS m/z=468.4 [M+H]⁺.

259: By general procedure 2. Obtained 177 mg (crude). LCMS m/z=368.4 [M+H]⁺.

260: By general procedure 1 using MP-CNBH₃ (1 equiv.) in MeOH. Obtained 210 mg, 77% yield. LCMS m/z=627.4 [M+H]⁺.

261: By general procedure 2. Obtained 200 mg. LCMS m/z=527.2 [M+H]⁺.

Compound B147: By general procedure 6. Obtained 40 mg, 20.8% yield. LCMS m/z=662.4 [M+H]⁺.

### Compound B86: (E/Z)-2-(5-((1-(2,6-dimethoxy-4-(1,4,5-trimethyl-6-oxo-1,6-dihydropyridin-3-yl)benzyl)piperidin-4-yl)methoxy)-1-methyl-1,2,3,4-tetrahydroisoquinoline-2-carbonyl)-4,4-dimethylpent-2-enenitrile

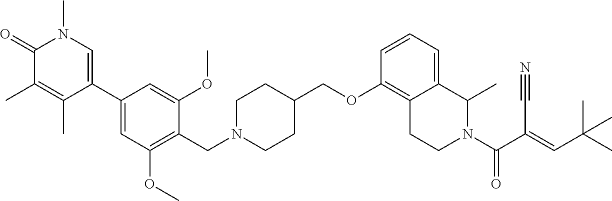 [SMILES: COc1cc(-c2cn(C)c(=O)c(C)c2C)cc(OC)c1CN1CCC(COc2cccc3c2CCN(C(=O)/C(C#N)=C/C(C)(C)C)C3C)CC1]

Prepared by following general procedure 6 using (E/Z)-2-cyano-4,4-dimethylpent-2-enoic acid (1.5 eq) in DMF. Obtained 12 mg, 15% yield. LCMS m/z=681.4 [M+H]⁺.

### (E/Z)-2-(5-(((R)-1-(2,6-dimethoxy-4-(1,4,5-trimethyl-6-oxo-1,6-dihydropyridin-3-yl)benzyl)pyrrolidin-3-yl)oxy)-1-methyl-1,2,3,4-tetrahydroisoquinoline-2-carbonyl)-4,4-dimethylpent-2-enenitrile (B133)

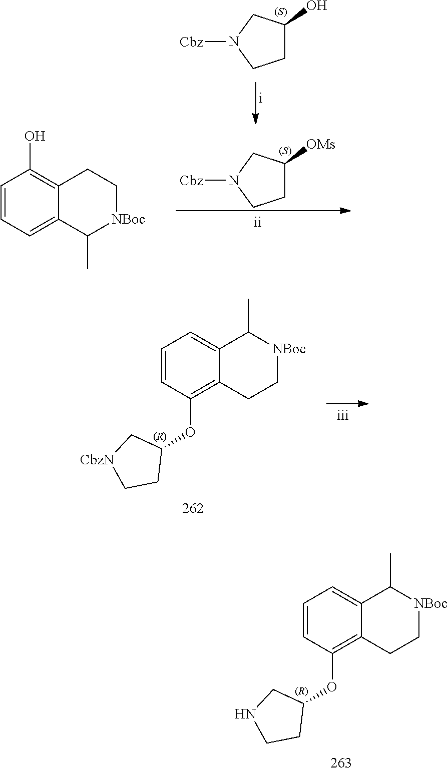

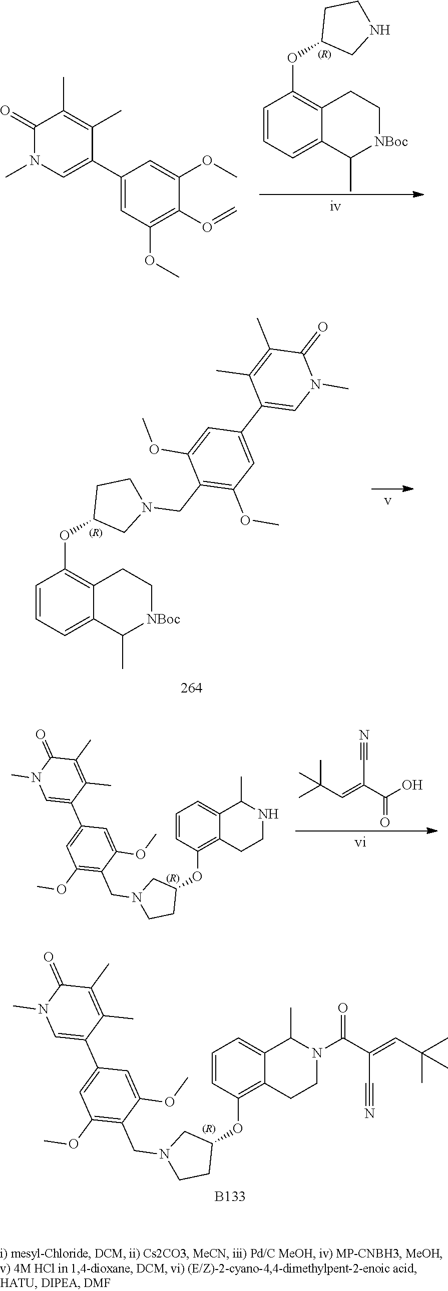

benzyl (S)-3-((methylsulfonyl)oxy)pyrrolidine-1-carboxylate: By general procedure 25. Obtained 1 g, 73.9% yield. LCMS m/z=300.2 [M+H]⁺.

262: By general procedure 26. Obtained 500 mg, 86% yield. LCMS m/z=367.2 [M-Boc]⁺.

263: By general procedure 27. Obtained 400 mg, 89% yield. LCMS m/z=333.4 [M+H]⁺.

264: By general procedure 1 using MP-CNBH₃ (1 equiv.) in MeOH. Obtained 500 mg, 78% yield. LCMS m/z=618.4 [M+H]⁺.

265: By general procedure 2 using 4M solution HCl in 1, 4-dioxane in DCM. Obtained 560 mg (crude). LCMS m/z=518.2 [M+H]⁺.

Compound B133: By general procedure 6 using (E/Z)-2-cyano-4,4-dimethylpent-2-enoic acid (2 eq.) in DMF. Obtained 6 mg, 3.3% yield. LCMS m/z=653.4 [M+H]⁺.

### (E/Z)-2-(2-(3-(((S)-1-(2,6-dimethoxy-4-(1,4,5-trimethyl-6-oxo-1,6-dihydropyridin-3-yl)benzyl)417yrrolidine-3-yl)oxy)phenyl)pyrrolidine-1-carbonyl)-4,4-dimethylpent-2-enenitrile (B67)

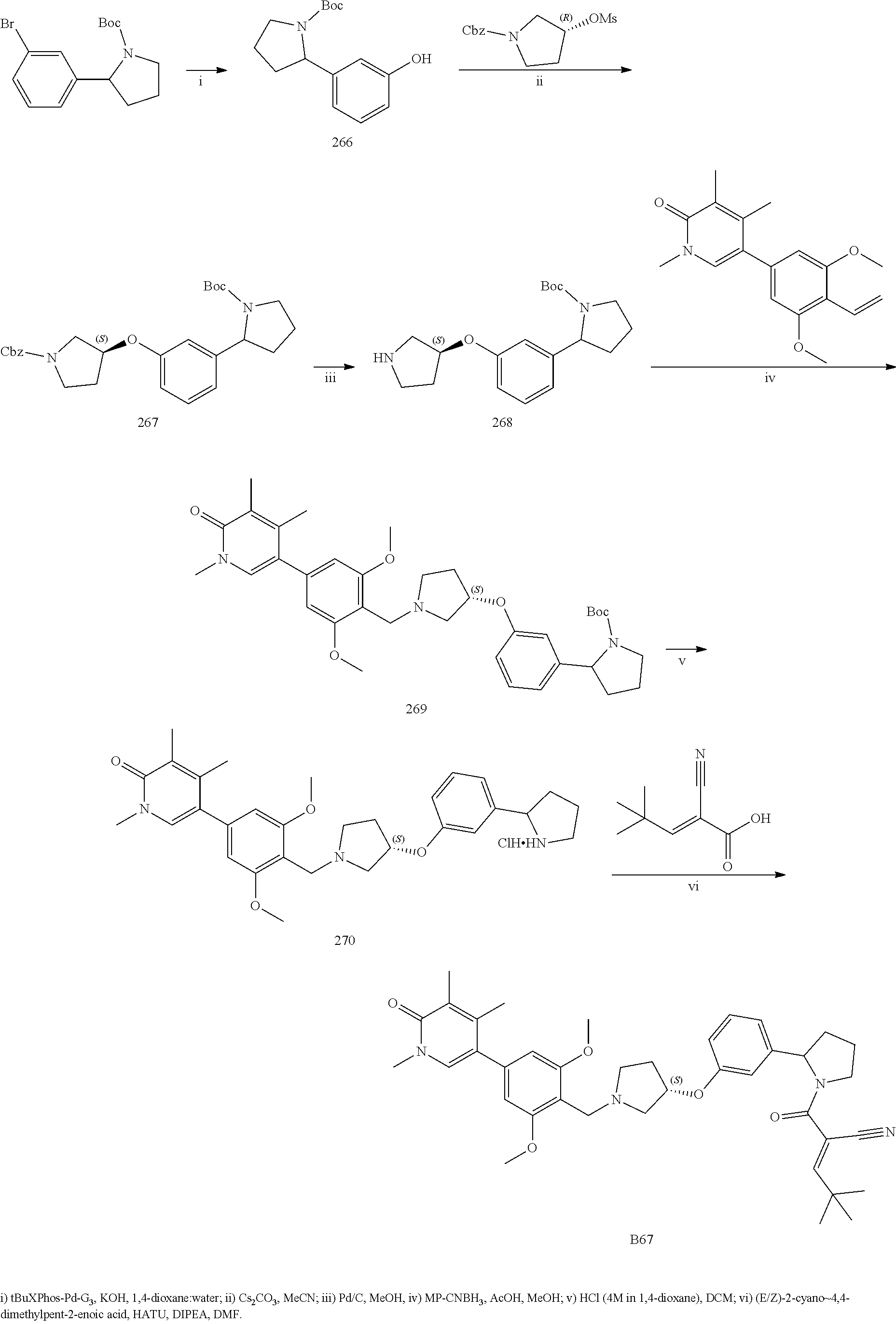

266: By general procedure 24 with KOH (6 eq) in 1,4-dioxane:water. Obtained 560 mg, 45% yield. LCMS m/z=208 [M+H]⁺.

267: By general procedure 26 using benzyl ®-3-((methylsulfonyl)oxy)pyrrolidine-1-carboxylate (2 eq) in MECN. Obtained 750 mg, 78% yield. LCMS m/z=387.2 [M+H-Boc]⁺.

268: By general procedure 27 using Pd/C (w/w) in methanol. Obtained 400 mg, 76% yield. LCMS m/z=333.2 [M+H-Boc]⁺.

269: By general procedure 14 using MP-CNBH₃ (w/w) in MeOH. Obtained 550 mg, 73% yield. LCMS m/z=618.3 [M+H]⁺.

270: By general procedure 2. Obtained 80 ng (crude). LCMS m/z=518.2 [M+H]⁺.

Compound B67: By general procedure 6 using (E/Z)-2-cyano-4,4-dimethylpent-2-enoic acid (1.2 eq) in DMF. Obtained 95 mg, 24.4% yield. LCMS m/z=653.2 [M+H]⁺.

### (E/Z)-2-(2-(3-((1-(2,6-dimethoxy-4-(1,4,5-trimethyl-1-oxo-1,6-dihydropyridin-3-yl)benzoyl)piperidin-4-yl)oxy)phenyl)pyrrolidine-1-carbonyl)-4,4-dimethylpent-2-enenitrile (B79)

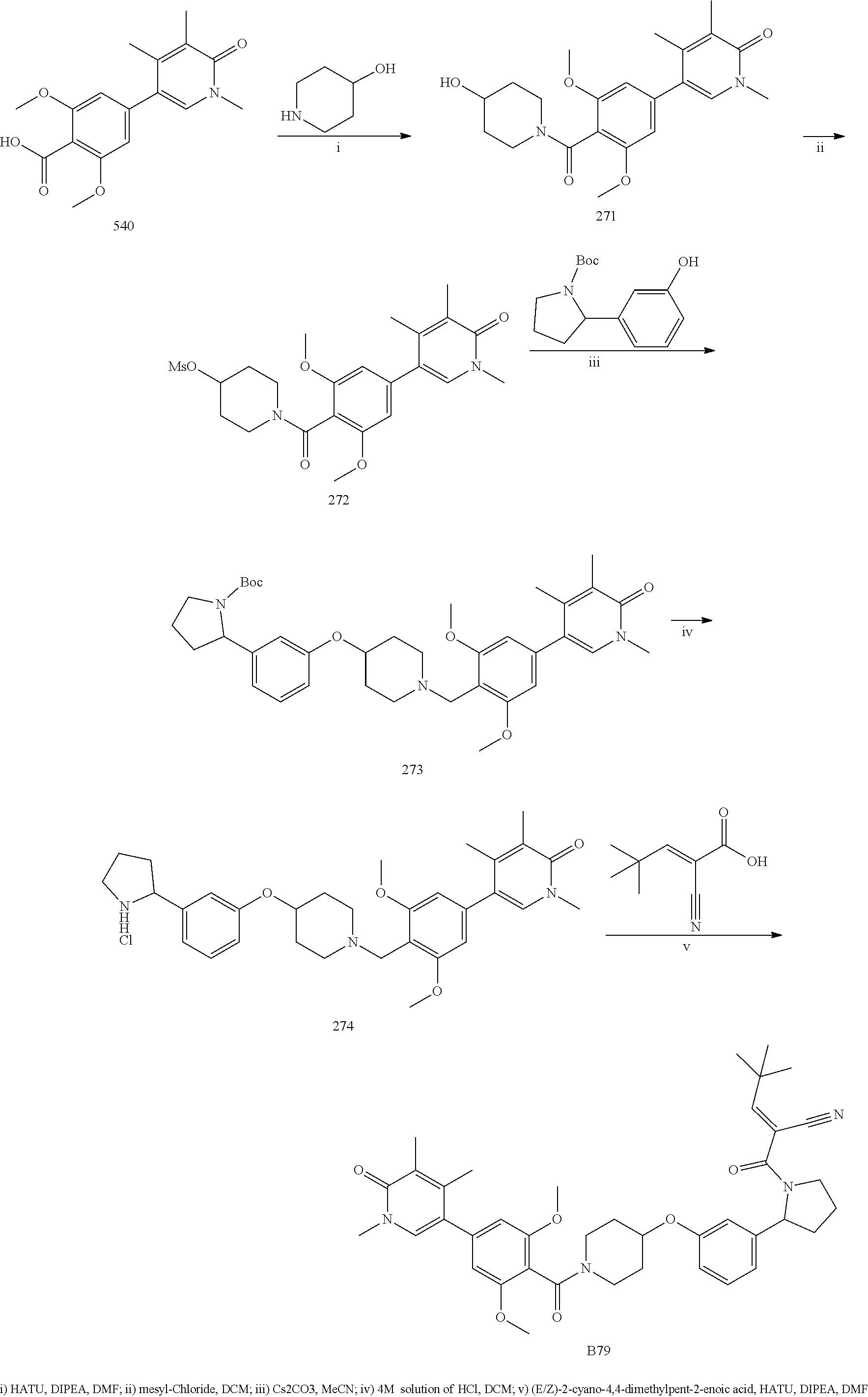

540: Prepared as in WO2021/178920

271: By general procedure 6 using piperidin-4-ol (1.5 eq) in DMF. Obtained 700 mg, 90% yield. LCMS m/z=401.2 [M+H]⁺.

272: By general procedure 25 using mesyl-chloride (1.5 eq) in DCM. Obtained 700 mg, 86% yield. LCMS m/z=479.0 [M+H]⁺.

273: By general procedure 26 using tert-butyl 2-(3-hydroxyphenyl)pyrrolidine-1-carboxylate (2 eq) in MeCN. Obtained 700 mg, 74% yield. LCMS m/z=646.6 [M+H]⁺.

274: By general procedure 2. Obtained 600 mg (crude). LCMS m/z=546.7 [M+H]⁺.

Compound B79: By general procedure 6 using (E/Z)-2-cyano-4,4-dimethylpent-2-enoic acid (1.5 eq) in DMF. Obtained 20 mg, Ile % yield. LCMS m/z=681.2 [M+H]⁺.

### Compounds B197 and B198 (Diastereomers of Unknown Absolute Configuration)

### Synthesis of both diastereomers of (E/Z)-2-(5-(((S)-4-(2,6-dimethoxy-4-(1,4,5-trimethyl-6-oxo-1,6-dihydropyridin-3-yl)benzyl)-2-methylpiperazin-1-yl)methyl)-1-methyl-1,2,3,4-tetrahydroisoquinoline-2-carbonyl)-4,4dimethylpent-2-enenitrile

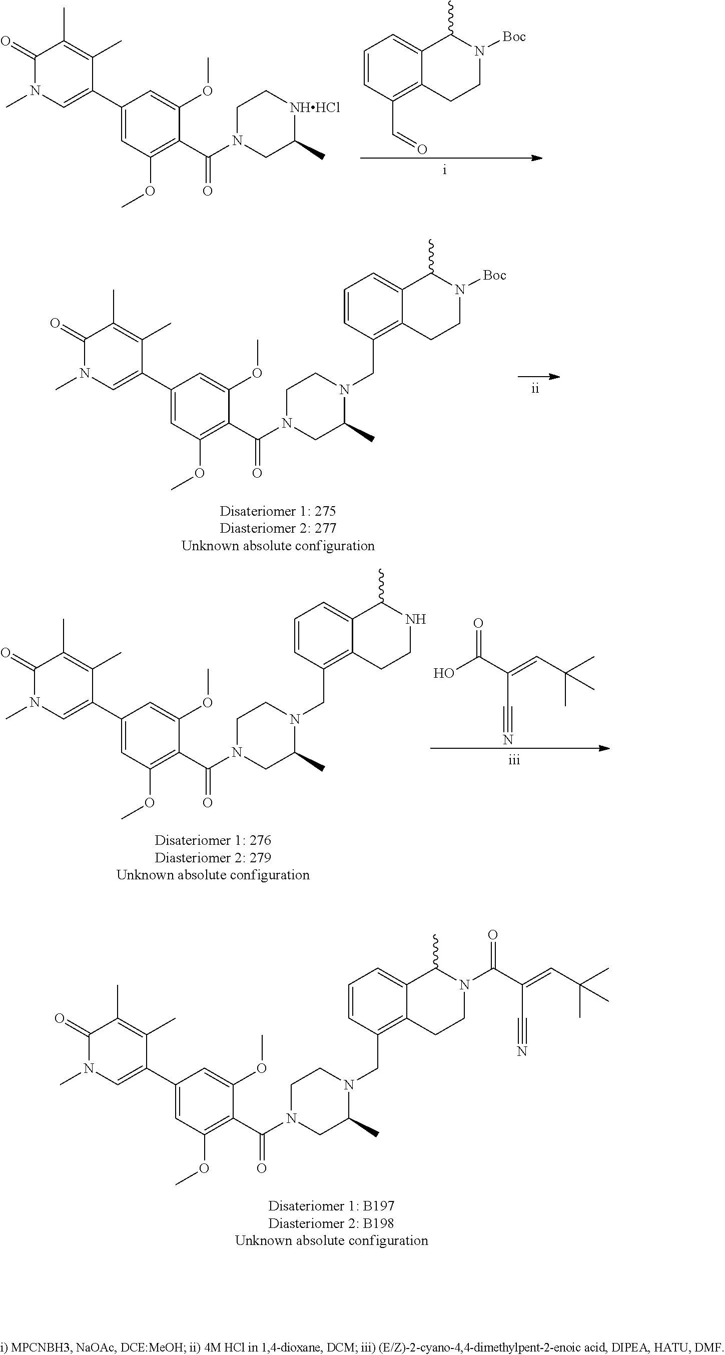

275: By general procedure 14a using MP-CNBH₃ (w/w) in DCE: MeOH. Obtained 200 mg, 59.4% yield. LCMS m/z=645.4 [M+H]⁺.

276: By general procedure 2. Obtained 180 mg, 95% yield. LCMS m/z=545.5 [M+H]⁺.

Compound B197: By general procedure 6 using (E/Z)-2-cyano-4,4-dimethylpent-2-enoic acid (2 eq) in DMF. Obtained (48.8 mg, 0.071 mmol, 48.5% yield). LCMS m/z=680.4 [M+H]⁺

277: By general procedure 14a using MP-CNBH₃ (w/w) in DCE:MeOH. Obtained 205 mg 60.1% yield. LCMS m/z=645.4 [M+H]⁺.

278: By general procedure 2. Obtained 160 mg (crude). LCMS m/z=545.5 [M+H]⁺.

Compound B198: By general procedure 6 using (E/Z)-2-cyano-4,4-dimethylpent-2-enoic acid (2 eq) in DMF. Obtained 47.9 mg, 39.6% yield. LCMS m/z=680.4 [M+H]⁺.

### Compounds B199, B200, C33 and C34

### Synthesis of Enantiomers of (E/Z)-2-(7-((4-(2,6-dimethoxy-4-(1,4,5-trimethyl-6-oxo-1,6-dihydropyridin-3yl)benzyl)-4,7-diazaspiro[2.5]octan-7-yl)methyl)-1-methyl-1,2,3,4-tetrahydroisoquinoline-2-carbonyl)-4,4-dimethylpent-2-enenitrile and Enantiomers of 3-(7-((4-(2,6-dimethoxy-4(1,4,5-trimethyl-6-oxo-1,6-dihydropyridin-3-yl)benzyl)-4,7-diazaspiro[2.5]octan-7-yl)methyl)-1-methyl-3,4-dihydroisoquinolin-2(1H)-yl)-3-oxopropanenitrile

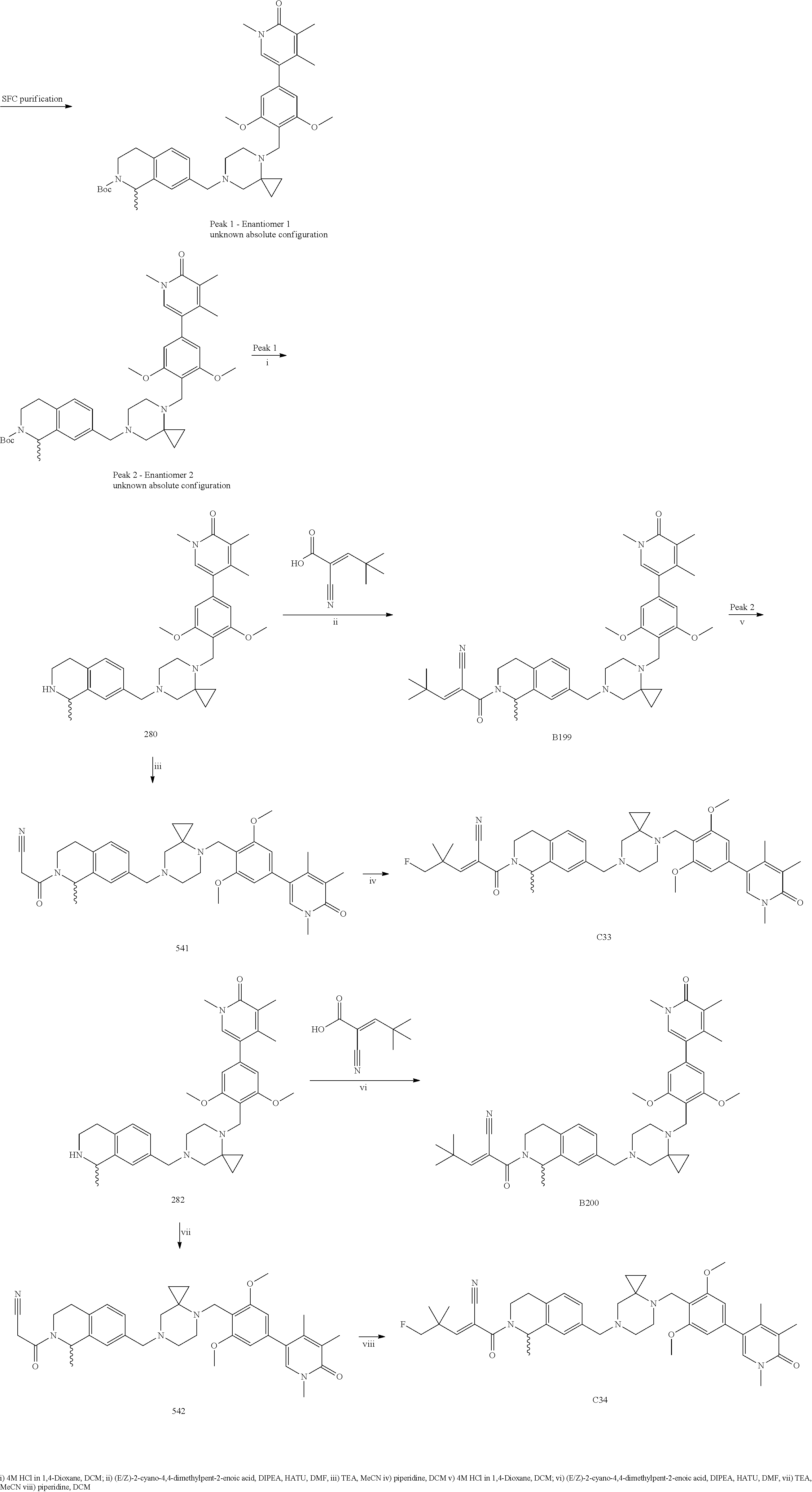

Racemic tert-butyl 7-((4-(2,6-dimethoxy-4-(1,4,5-trimethyl-6-oxo-1,6-dihydropyridin-3-yl)benzyl)-4,7-diazaspiro[2.5]octan-7-yl)methyl)-1-methyl-3,4-dihydroisoquinoline-2 (1H)-carboxylate (400 mg) was purified by SFC:

| | |
|---|---|
| Instrument | PIC 22-016 |
| Column | I Cellulose Z-(250*30) mm, 5 μm |
| Mobile Phase | CO₂: 0.5% isopropylamine in MeOH:MeCN [65:35] |
| Flow rate | 120 mL/min |
| Back pressure | 120 bar |
| Wavelength | 254 nm |
| Cycle time | 6 min |

Isolated Peak-1: 170 mg, 41.2% yield. LCMS m/z=657.4 [M+H]⁺ and Peak-2: 160 mg, 39.6% yield. LCMS m/z=657.4 [M+H]⁺

282: By general procedure 2. Obtained 138 mg (crude). LCMS m/z=557.3 [M+H]⁺.

280: By general procedure 2. Obtained 140 mg (crude). LCMS m/z=557.3 [M+H]⁺.

Compound B200: By general procedure 6 using (E/Z)-2-cyano-4,4-dimethylpent-2-enoic acid (2 eq) in DMF. Obtained 31 mg, 30.5% yield. LCMS m/z: 692.3 [M+H]⁺.

Compound B199: By general procedure 6 using (E/Z)-2-cyano-4,4-dimethylpent-2-enoic acid (2 eq) in DMF. Obtained (45 mg, 34.5% yield). LCMS m/z: 692.3 [M+H]⁺.

541: Prepared following general procedure 3 using peak 1 (intermediate 280) and 3-(3,5-dimethyl-1H-pyrazol-1-yl-3-oxopropanenitrile (1.2 eq) in MeCN. Obtained 320 mg, 58% yield. LCMS m/z=624.3 [M+H]⁺

Compound C33: Prepared following general procedure 4 using 3-fluoro-2,2-dimethylpropanal (4 eq) in DCM. Obtained 4 mg, 5.4% yield. LCMS m/z=710.3 [M+H]⁺.

542: Prepared following general procedure 3 using peak 2 (intermediate 282) and 3-(3,5-dimethyl-1H-pyrazol-1-yl)-3-oxopropanenitrile (1.2 eq) in MeCN. Obtained 300 mg, 53% yield. LCMS m/z=624.3 [M+H]⁺

Compound C34: Prepared following general procedure 4 using 3-fluoro-2,2-dimethylpropanal (4 eq) in DCM. Obtained 3.4 mg, 4.5% yield. LCMS m/z=710.3 [M+H]⁺.

### Compounds B201, B202 and B203, C35 and C36

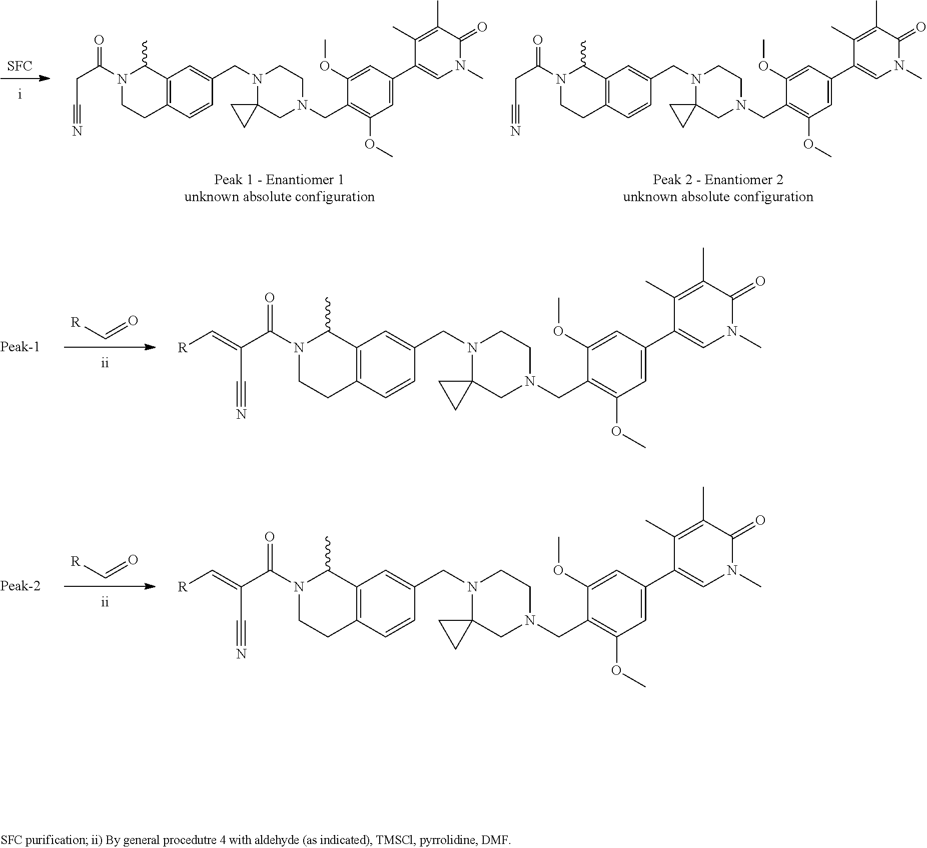

Racemic 3-(7-((7-(2,8-dimethoxy-4-(1,4,5-trimethyl-8-oxo-1,8-dihydropyridin-3-yl)benzyl)-4,7-diazaspiro[2.5]octan-4-yl)methyl)-1-methyl-3,4-dihydroisoquinolin-2(1H)-yl)-3-oxopropanenitrile (240 mg) was purified by SFC:

| | |
|---|---|
| Instrument | PIC 22-017 |
| Column | WHELK RR-(250*30)mm, 5 μm |
| Mobile Phase | CO₂: 0.5% isopropylamine in MeOH:MeCN [60:40] |
| Flow rate | 50 mL/min |
| Back pressure | 100 bar |
| Wavelength | 254 nm |
| Cycle time | 13 min |

Isolated Peak-1: 80 mg, 33.1% yield. LCMS m/z=624.4 [M+H]⁺ and Peak-2: 50 mg, 20.7% yield. LCMS m/z=624.4 [M+H]⁺

The following compounds were prepared using peak from the chiral SFC of 3-(7-((7-(2,7-dimethoxy-4-(1,4,5-trimethyl-6-oxo-1,6-dihydropyridin-3-yl)benzyl)-4,7-diazaspiro[2.5]octan-4-yl)methyl)-1-methyl-3,4-dihydroisoquinolin-2(1H)-yl)-3-oxopropanenitrile and the relevant aldehyde

| | | | LCMS m/z |
|---|---|---|---|
| Cmpd | | Procedure | [M + H]⁺/Yield |
| B201 | 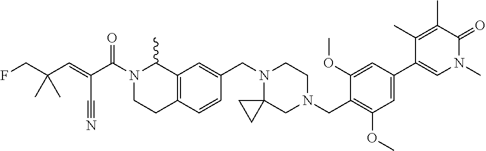 [SMILES: COc1cc(-c2cn(C)c(=O)c(C)c2C)cc(OC)c1CN1CCN(Cc2ccc3c(c2)C(C)N(C(=O)/C(C#N)=C/C(C)(C)CF)CC3)C2(CC2)C1] | 4 | 710.4/17.2% |
| | Enantiomer 1 of (E/Z)-2-(7-((7-(2,6-dimethoxy-4-(1,4,5-trimethyl-6-oxo-1,6 | | |
| | dihydropyridin-3-yl)benzyl)-4,7-diazaspiro[2.5]octan-4-yl)methyl)-1-methyl- | | |
| | 1,2,3,4-tetrahydroisoquinoline-2-carbonyl)-5-fluoro-4,4-dimethylpent- | | |
| | 2-enenitrile | | |
| B202 | 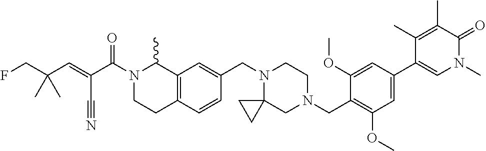 [SMILES: COc1cc(-c2cn(C)c(=O)c(C)c2C)cc(OC)c1CN1CCN(Cc2ccc3c(c2)C(C)N(C(=O)/C(C#N)=C/C(C)(C)CF)CC3)C2(CC2)C1] | 4 | 710.4/13.1% |
| | Enantiomer 2 of (E/Z)-2-(7-((7-(2,6-dimethoxy-4-(1,4,5-trimethyl-6-oxo-1,6- | | |
| | dihydropyridin-3-yl)benzyl)-4,7-diazaspiro[2.5]octan-4-yl)methyl)-1-methyl- | | |
| | 1,2,3,4-tetrahydroisoquinoline-2-carbonyl)-5-fluoro-4,4-dimethylpent- | | |
| | 2-enenitrile | | |
| B203 | 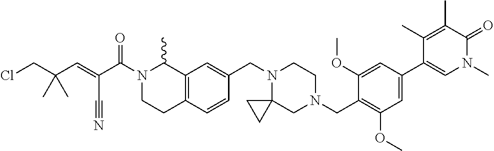 [SMILES: COc1cc(-c2cn(C)c(=O)c(C)c2C)cc(OC)c1CN1CCN(Cc2ccc3c(c2)C(C)N(C(=O)/C(C#N)=C/C(C)(C)CCl)CC3)C2(CC2)C1] | 4 | 726.2/1.6% |
| | Enantiomer 1 of (E/Z)-5-chloro-2-(7-((7-(2,6-dimethoxy-4-(1,4,5-trimethyl-6- | | |
| | oxo-1,6-dihydropyridin-3-yl)benzyl)-4,7-diazaspiro[2.5]octan-4-yl)methyl)-1- | | |
| | methyl-1,2,3,4-tetrahydroisoquinoline-2-carbonyl)-4,4-dimethylpent-2-enenitrile | | |
| C35 | 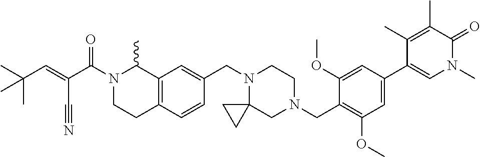 [SMILES: COc1cc(-c2cn(C)c(=O)c(C)c2C)cc(OC)c1CN1CCN(Cc2ccc3c(c2)C(C)N(C(=O)/C(C#N)=C/C(C)(C)C)CC3)C2(CC2)C1] | 4 | 692.4/24% |
| | Enantiomer 1 of (E/Z)-2-(7-((7-(2,6-dimethoxy-4-(1,4,5-trimethyl-6-oxo-1,6- | | |
| | dihydropyridin-3-yl)benzyl)-4,7-diazaspiro[2.5]octan-4-yl)methyl)-1-methyl- | | |
| | 1,2,3,4-tetrahydroisoquinoline-2-carbonyl)-4,4-dimethylpent-2-enenitrile | | |
| C36 | 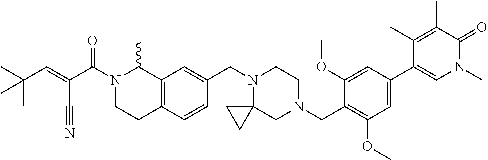 [SMILES: COc1cc(-c2cn(C)c(=O)c(C)c2C)cc(OC)c1CN1CCN(Cc2ccc3c(c2)C(C)N(C(=O)/C(C#N)=C/C(C)(C)C)CC3)C2(CC2)C1] | 4 | 692.4/21% |
| | Enantiomer 2 of (E/Z)-2-(7-((7-(2,6-dimethoxy-4-(1,4,5-trimethyl-6-oxo-1,6- | | |
| | dihydropyridin-3-yl)benzyl)-4,7-diazaspiro[2.5]octan-4-yl)methyl)-1-methyl- | | |
| | 1,2,3,4-tetrahydroisoquinoline-2-carbonyl)-4,4-dimethylpent-2-enenitrile | | |
| C37 | 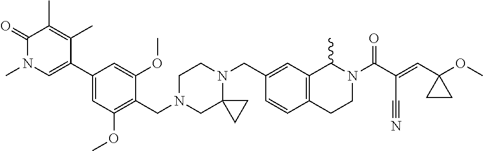 | 4 | 706.3/13% |
| | Enantiomer 1 of (E/Z)-2-(7-((7-(2,6-dimethoxy-4-(1,4,5-trimethyl-6-oxo- | | |
| | 1,6-dihydropyridin-3-yl)benzyl)-4,7-diazaspiro[2.5]octan-4-yl)methyl)-1- | | |
| | methyl-1,2,3,4-tetrahydroisoquinoline-2-carbonyl)-3-(1-methoxycyclo- | | |
| | propyl)acrylonitrile | | |
| C38 | 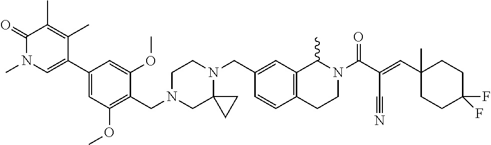 [SMILES: COc1cc(-c2cn(C)c(=O)c(C)c2C)cc(OC)c1CN1CCN(Cc2ccc3c(c2)C(C)N(C(=O)/C(C#N)=C/C2(C)CCC(F)(F)CC2)CC3)C2(CC2)C1] | 4 | 768.2/19% |
| | Enantiomer 1 of (E/Z)-3-(4,4-difluoro-1-methylcyclohexyl)-2-(7-((7-(2,6- | | |
| | dimethoxy-4-(1,4,5-trimethyl-6-oxo-1,6-dihydropyridin-3-yl)benzyl)- | | |
| | 4,7-diazaspiro[2.5]octan-4-yl)methyl)-1-methyl-1,2,3,4-tetrahydroiso- | | |
| | quinoline-2-carbonyl)acrylonitrile | | |
| C39 | 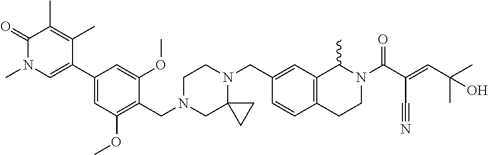 [SMILES: COc1cc(-c2cn(C)c(=O)c(C)c2C)cc(OC)c1CN1CCN(Cc2ccc3c(c2)C(C)N(C(=O)/C(C#N)=C/C(C)(C)O)CC3)C2(CC2)C1] | 4 | 694.6/3% |
| | Enantiomer 1 of (E/Z)-2-(7-((7-(2,6-dimethoxy-4-(1,4,5-trimethyl-6-oxo- | | |
| | 1,6-dihydropyridin-3-yl)benzyl)-4,7-diazaspiro[2.5]octan-4-yl)methyl)-1- | | |
| | methyl-1,2,3,4-tetrahydroisoquinoline-2-carbonyl)-4-hydroxy-4-methyl- | | |
| | pent-2-enenitrile | | |
| C40 | 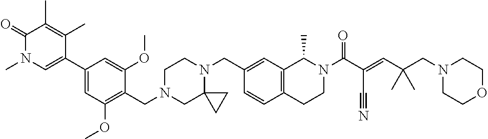 [SMILES: COc1cc(-c2cn(C)c(=O)c(C)c2C)cc(OC)c1CN1CCN(Cc2ccc3c(c2)[C@H](C)N(C(=O)/C(C#N)=C/C(C)(C)CN2CCOCC2)CC3)C2(CC2)C1] | 4 | 777.4/20% |
| | Enantiomer 1 of (E/Z)-2-(7-((7-(2,6-dimethoxy-4-(1,4,5-trimethyl-6-oxo- | | |
| | 1,6-dihydropyridin-3-yl)benzyl)-4,7-diazaspiro[2.5]octan-4-yl)methyl)- | | |
| | 1-methyl-1,2,3,4-tetrahydroisoquinoline-2-carbonyl)-4,4-dimethyl-5- | | |
| | morpholinopent-2-enenitrile | | |
| C41 | 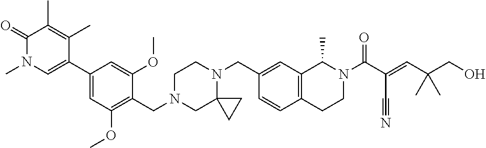 [SMILES: COc1cc(-c2cn(C)c(=O)c(C)c2C)cc(OC)c1CN1CCN(Cc2ccc3c(c2)[C@H](C)N(C(=O)/C(C#N)=C/C(C)(C)CO)CC3)C2(CC2)C1] | 4 | 708.2/7% |
| | Enantiomer 1 of (E/Z)-2-(7-((7-(2,6-dimethoxy-4-(1,4,5-trimethyl-6-oxo- | | |
| | 1,6-dihydropyridin-3-yl)benzyl)-4,7-diazaspiro[2.5]octan-4-yl)methyl)-1- | | |
| | methyl-1,2,3,4-tetrahydroisoquinoline-2-carbonyl)-5-hydroxy-4,4-dimeth- | | |
| | ylpent-2-enenitrile | | |

### (E/Z)-2-(2-(3-((4-(2,6-dimethoxy-4-(1,4,5-trimethyl-6-oxo-1,6-dihydropyridin-3-yl)benzoyl)piperazin-1-yl)methyl)phenyl)pyrrolidine-1-carbonyl)-4,4-dimethylpent-2-enenitrile (B46)

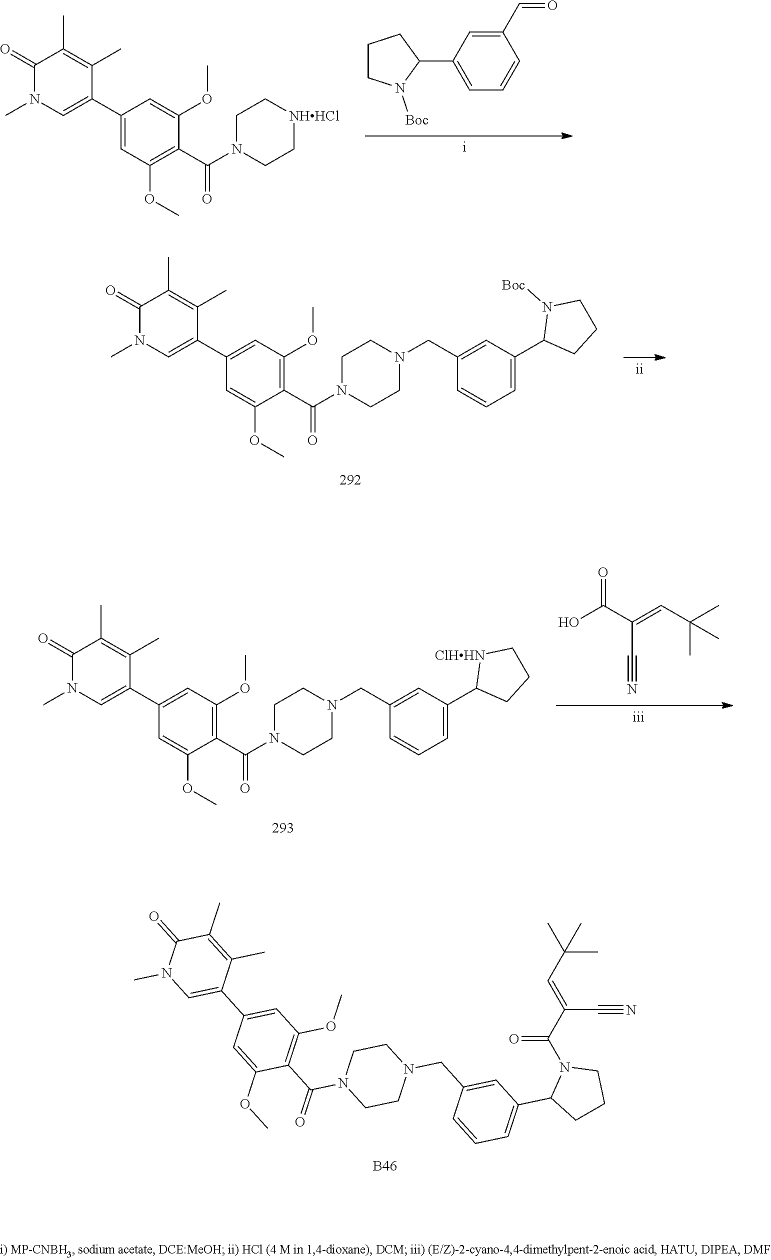

292: By general procedure 14 using MPCNBH₃ (w/w). Obtained 1 g, 29% yield. LCMS m/z=645.6 [M+H]⁺.

293: By general procedure 2. Obtained 900 mg, crude. LCMS m/z=545.2 [M+H].

Compound B46: By general procedure 6 using (E/Z)-2-cyano-4,4-dimethylpent-2-enoic acid (1.5 eq) in DMF. Obtained 45 mg, 19.6% yield. LCMS m/z=680.0 [M+H]⁺.

### Compound (E/Z)-2-(2-(4-(4-(2,6-dimethoxy-4-(1,4,5-trimethyl-6-oxo-1,6-dihydropyridin-3-yl)benzyl)piperazine-1-carbonyl)phenyl)pyrrolidine-1-carbonyl)-4,4-dimethylpent-2-enenitrile (B55)

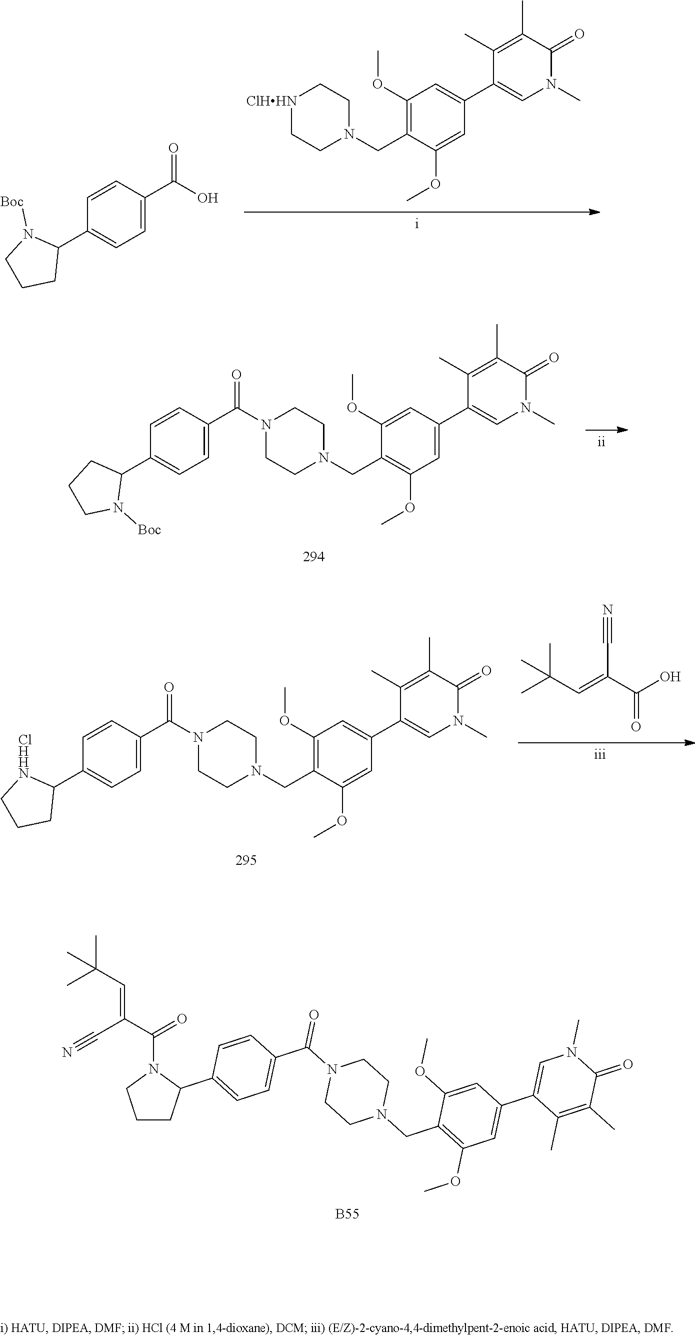

294: By general procedure 6 using 4-(1-(tert-butoxycarbonyl)pyrrolidin-2-yl)benzoic acid (1 eq) in DMF. Obtained 140 mg, 32% yield. LCMS m/z=645.4 [M+H]⁺.

295: By general procedure 2. Obtained 130 mg (crude). LCMS m/z=545.6 [M+H]⁺

B55: By general procedure 6 using (E/Z)-2-cyano-4,4-dimethylpent-2-enoic acid (2 eq) in DMF. Obtained 28 mg, 20% yield. LCMS m/z=680.4 [M+H]⁺.

### (E/Z)-2-(2-(3-(((S)-1-(2,6-dimethoxy-4-(1,4,5-trimethyl-6-oxo-1,6-dihydropyridin-3-yl)benzyl)pyrrolidin-3-yl)oxy)phenyl)pyrrolidine-1-carbonyl)-4,4-dimethylpent-2-enenitrile (B54)

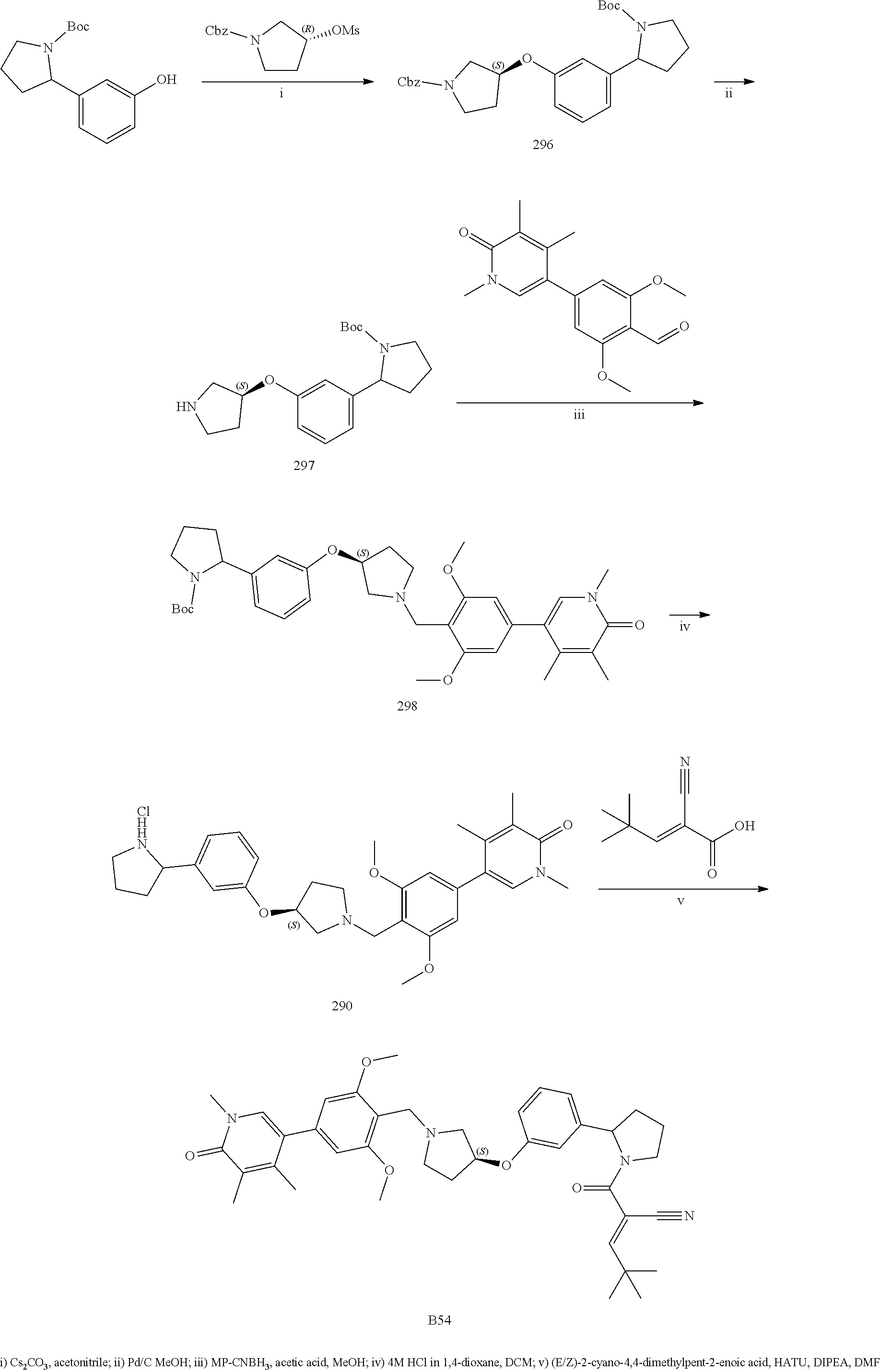

296: By general procedure 26 using Cs₂CO₃ (3 eq) in MeCN. Obtained 770 mg, 47% yield. LCMS m/z=387.2 [M+H-Boc]⁺.

297: By general procedure 27 using Pd/C (w/w) in methanol. Obtained 180 mg, 72% yield. LCMS m/z=333.4 [M+H-Boc]⁺.

298: By general procedure 1 using MPCNBH₃ (w/w) in MeOH. Obtained 250 mg, 74% yield. LCMS m/z=018.4 [M+H]⁺.

290: By general procedure 2 using 4M HCl in 1,4-dioxane in HCl, in DCM. Obtained 92 mg (crude). LCMS m/z=518.2 [M+H]⁺.

Compound B54: By general procedure 6 using (E/Z)-2-cyano-4,4-dimethylpent-2-enoic acid (1.2 eq) in DMF. Obtained 70 mg, 27% yield. LCMS m/z=653.4 [M+H]⁺.

### (E/Z)-2-(2-(3-(6-(2,6-dimethoxy-4-(1,4,5-trimethyl-6-oxo-1,6-dihydropyridin-3-yl)benzyl)-2,6-diazaspiro[3.3]heptan-2-yl)phenyl)pyrrolidine-1-carbonyl)-4,4-dimethylpent-2-enenitrile (58)

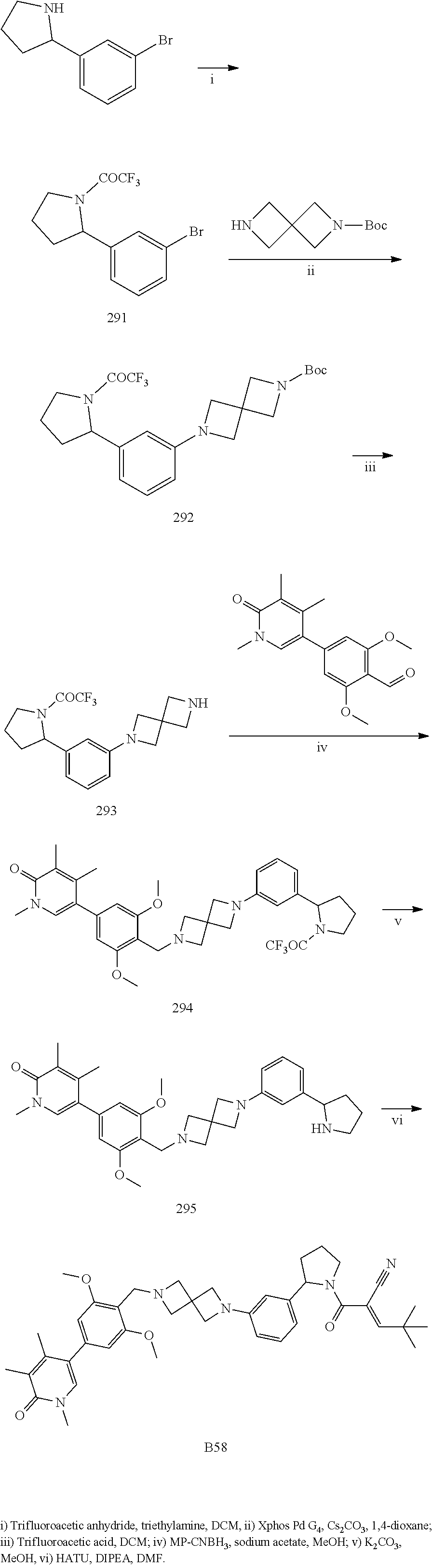

291: To a stirred solution of 2-(3-bromophenyl)pyrrolidine (7.0 g, 31.0 mmol, 1 eq) in DCM (50 mL) at 0° C. was added TEA (8.83 ml, 01.9 mmol, 2 eq), followed by TFAA (6.58 ml, 40.4 mmol, 1.5 eq). The reaction mixture was stirred at RT overnight before being quenched with water and extracted with DCM. The combined organic layers were concentrated in vacuo and the resulting residue was purified by silica gel column chromatography (gradient=5% EtOAc in hexane). The appropriate fractions were concentrated in vacuo to afford 1-(2-(3-bromophenyl)pyrrolidin-1-yl)-2,2,2-trifluoroethan-1-one (6.0 g, 18.63 mmol, 60.2% yield). LCMS m/z=324.0 [M+H]⁺.

292: Prepared by following general procedure 21 using tert-butyl 2,6-diazaspiro[3.3]heptane-2-carboxylate (1 eq) and XPhosPdG4 (0.1 eq) in 1,4-dioxane. Obtained 550 mg, 68% yield. LCMS m/z=440.2 [M+H]⁺.

293: By general procedure 13. Obtained 500 mg, Crude. LCMS m/z=340.2 [M+H]⁺.

294: By general procedure 14 using MPCNBH₃ (w/w) in DCE:MeOH. Obtained 150 mg, 14% yield. LCMS m/z=625.3 [M+H]⁺.

295: By general procedure 20 using K₂CO₃ (5 eq) in MeOH-water. Obtained 130 mg (crude). LCMS m/z=529.2 [M+H]⁺.

Compound B58: By general procedure 6 using (E/Z)-2-cyano-4,4-dimethylpent-2-enoic acid (2eq) in DMF. Obtained 16 mg, 13% yield. LCMS m/z=664.4 [M+H]⁺.

### (E/Z)-2-(2-(3-((1-(2,6-dimethoxy-4-(1,4,5-trimethyl-6-oxo-1,6-dihydropyridin-3-yl)benzyl)azetidin-3-yl)oxy)phenyl)pyrrolidine-1-carbonyl)-4,4-dimethylpent-2-enenitrile (B62)

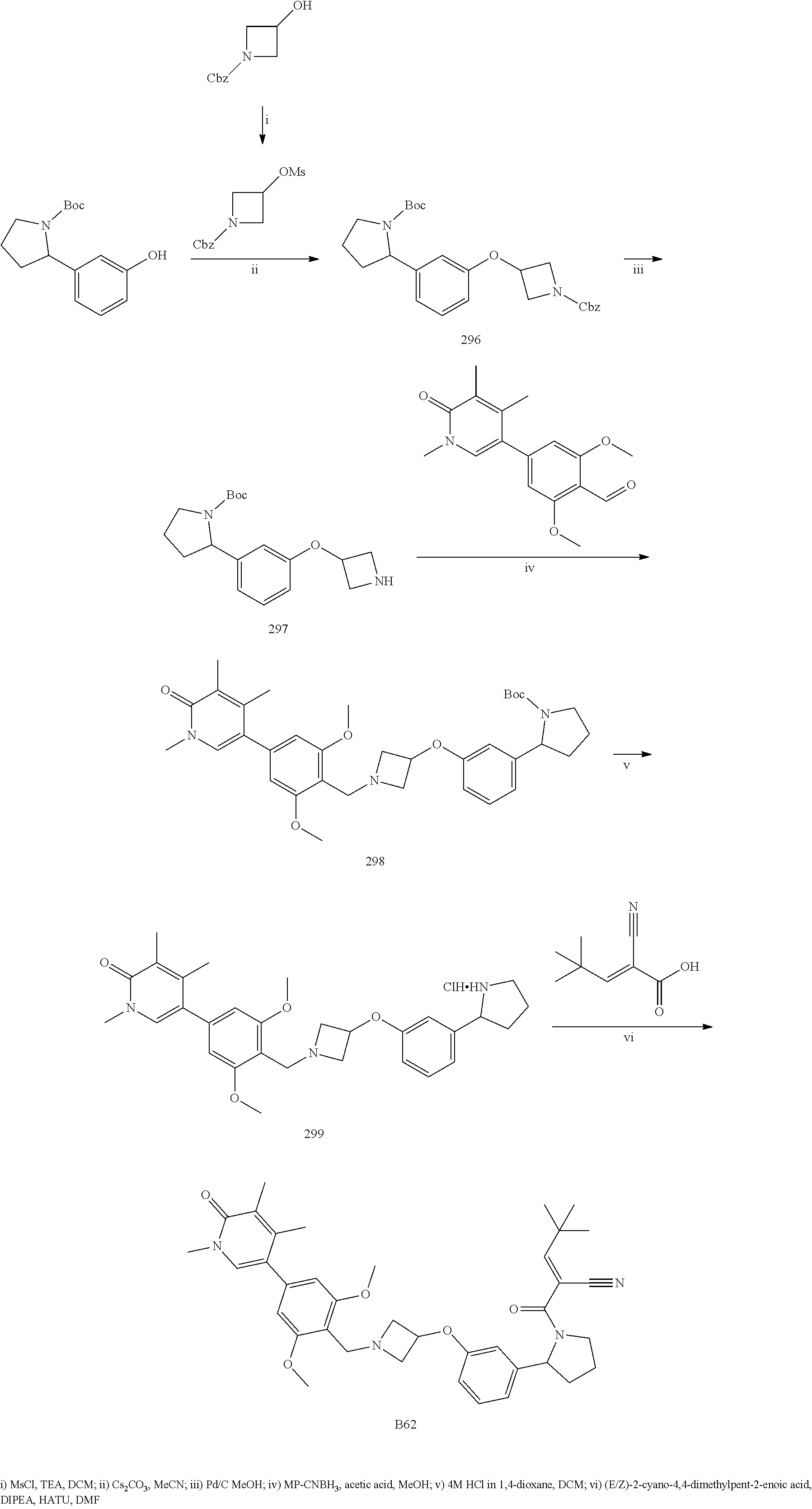

### Synthesis of benzyl 3-((methylsulfonyloxy)azetidine-1-carboxylate

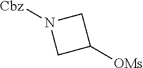 [SMILES: CS(=O)(=O)OC1CN(C(=O)OCc2ccccc2)C1]

By general procedure 25 using mesyl-CI (2 eq) in DCM. Obtained 6.4 g, 93% yield.

296: By general procedure 26. Obtained 770 mg, 47% yield. LCMS m/z=353.2 [M+H-Boc]⁺.

297: By general procedure 27. Obtained 250 mg. LCMS m/z=319.2 [M+H]⁺.

298: By general procedure 14 using MPCNBH₃ (w/w) in DCE:MeOH. Obtained 157 mg, 16% yield. LCMS m/z=604.3 [M+H]⁺.

299: By general procedure 2. Obtained 140 mg, crude. LCMS m/z=504.4 [M+H]⁺.

Compound B62: By general procedure 6 using (E/Z)-2-cyano-4,4-dimethylpent-2-enoic acid (2 eq). Obtained 9.3 mg, 6% yield. LCMS m/z 639.3 [M+H]⁺.

### (E/Z)-3-(6-bromopyridin-2-yl)-2-(6-((1-(2,6-dimethoxy-4-(1,4,5-trimethyl-6-oxo-1,6-dihydropyridin-3-yl)benzyl)azetidin-3-yl)oxy)-1-methyl-1,2,3,4-tetrahydroisoquinoline-2-carbonyl)acrylonitrile (B57)

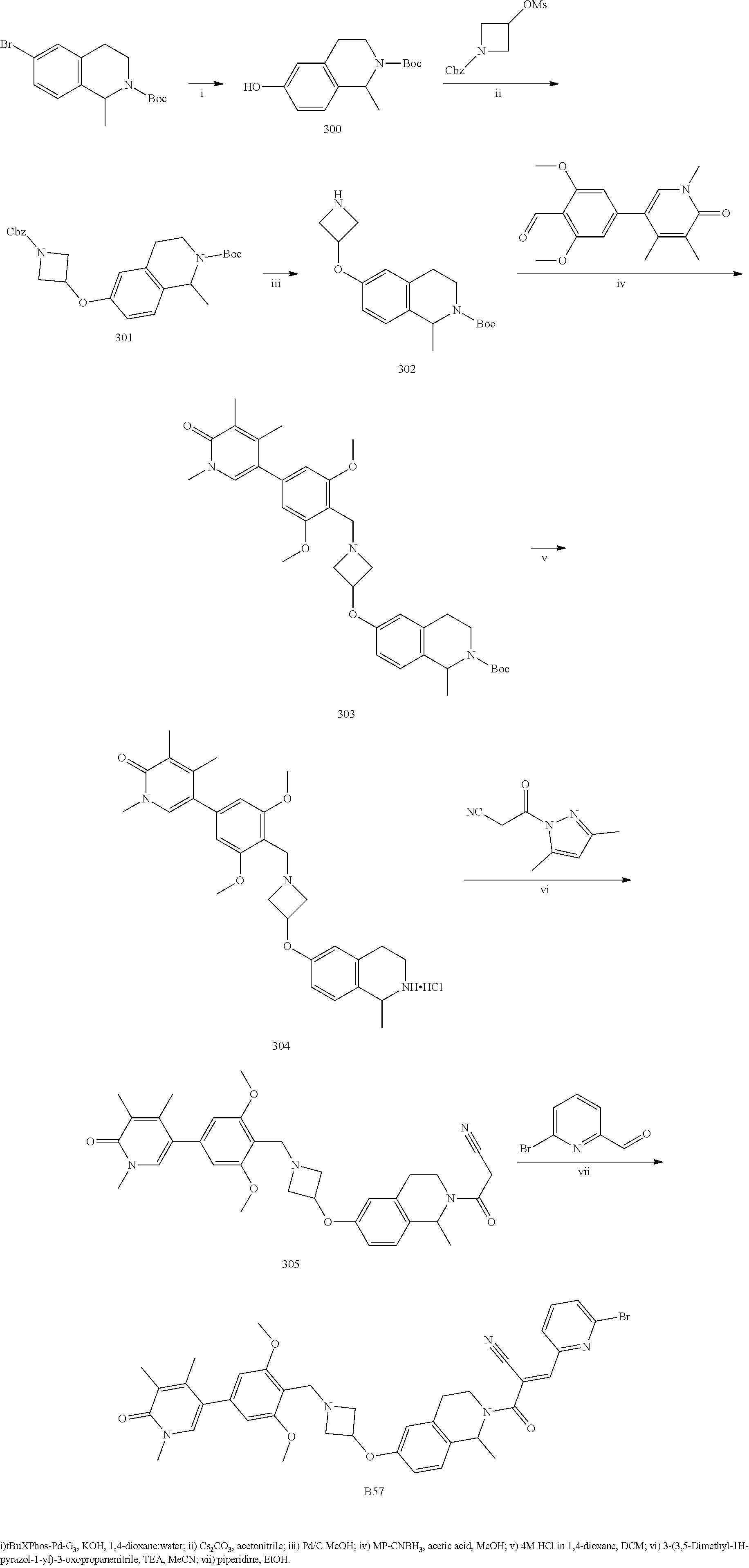

300: By general procedure 24 using KOH (6 eq) in 1,4-dioxane:water (4:1). Obtained 1 g, 61% yield. LCMS m/z=208.2 [M+H−56]⁺.

301: By general procedure 26 using Cs₂CO₃ (3 eq) in MeCN. Obtained 168 mg, 38% yield. LCMS m/z=353.2 [M+H-Boc]⁺.

302: By general procedure 27. Obtained 250 mg (crude). LCMS m/z=319.2 [M+H]⁺.

303: By general procedure 1 using MPCNBH₃ (w/w). Obtained 450 mg (crude). LCMS m/z=604.4 [M+H]⁺.

304: By general procedure 2. Obtained 340 mg (crude). LCMS m/z=504.5 [M+H]⁺.

305: By general procedure 3 using 3-(3,5-dimethyl-1H-pyrazol-1-yl)-3-oxopropanenitrile (1.2 eq) in MeCN. Obtained 265 mg, 70% yield. LCMS m/z=571.6 [M+H].

Compound B57: By general procedure 4 using 6-bromopicolinaldehyde (3 eq) in Ethanol. Obtained 1.8 mg, 1% yield. LCMS m/z=739.2 [M+H].

### (E2Z)-2-(5-(8-(2,6-dimethoxy-4-(1,4,5-trimethyl-6-oxo-1,6-dihydropyridin-3-yl)benzyl)-2,8-diazaspiro[4.5]decan-2-yl)-1-methyl-1,2,3,4-tetrahydroisoquinoline-2-carbonyl)-4,4-dimethylpent-2-enenitrile (B63)

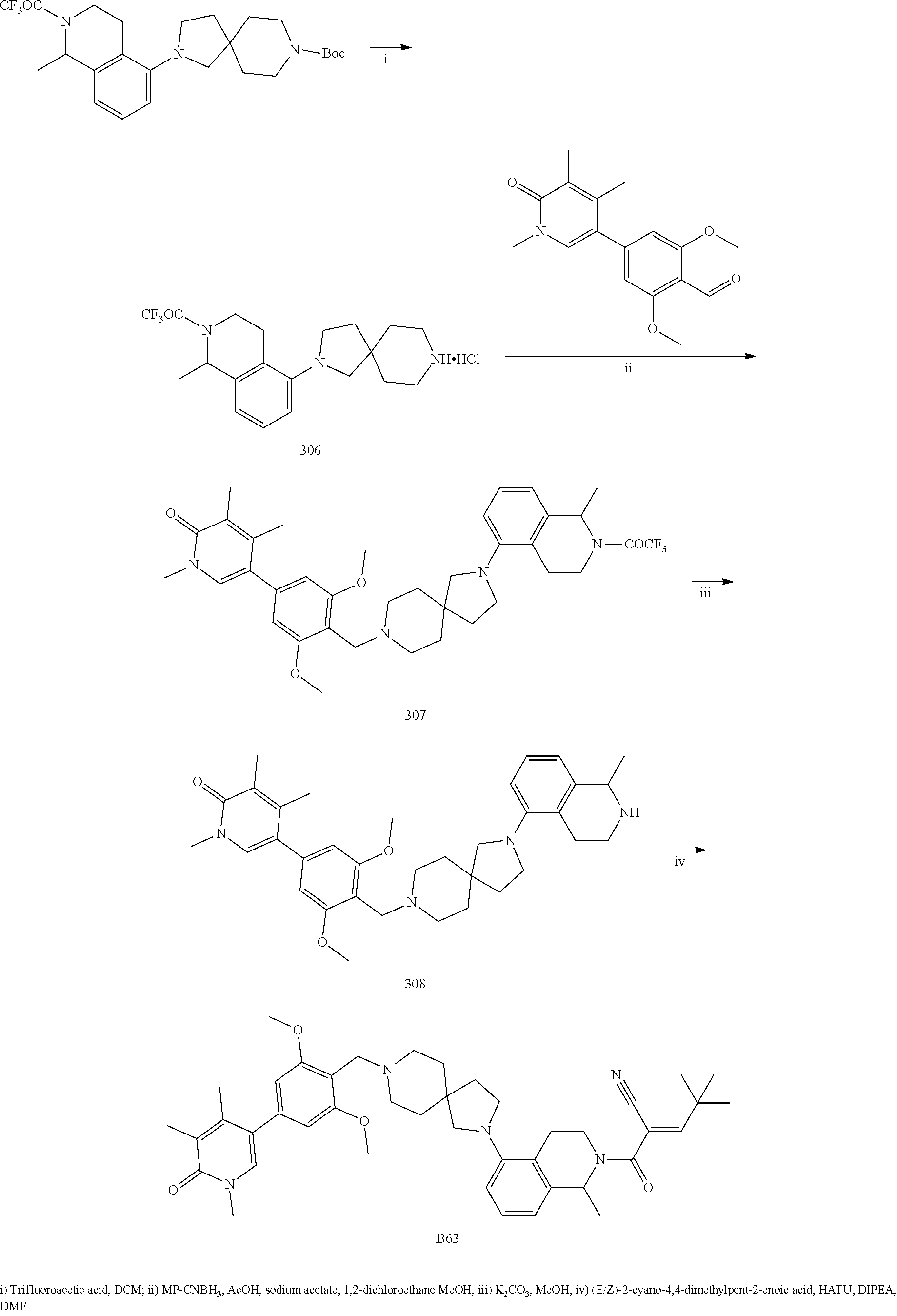

306: By general procedure 2. Obtained 270 mg, Crude. LCMS m/z=382.2 [M+H]⁺.

307: By general procedure 14b using MPCNBH₃ (w/w) in DCE: MeOH. Obtained 400 mg, 72% yield. LCMS m/z=667.2 [M+H]⁺.

308: By general procedure 20 using K2CO₃ (2.5 eq) in MeOH: water (4:1). Obtained 150 mg, crude. LCMS m/z=571.2 [M+H]⁺.

Compound B63: By general procedure 6 using (E/Z)-2-cyano-4,4-dimethylpent-2-enoic acid (1.5 eq) in DMF. Obtained 55 mg, 55% yield. LCMS m/z=706.4 [M+H]⁺.

### (E/Z)-2-(5-((1-(2,6-dimethoxy-4-(1,4,5-trimethyl-6-oxo-1,6-dihydropyridin-3-yl)benzyl)piperidin-4-yl)methyl)-1-methyl-1,2,3,4-tetrahydroisoquinoline-2-carbonyl)-4,4-dimethylpent-2-enenitrile (B156)

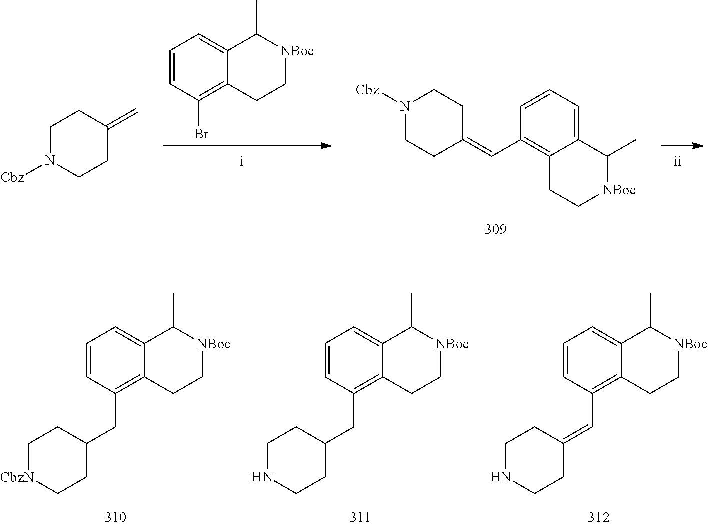

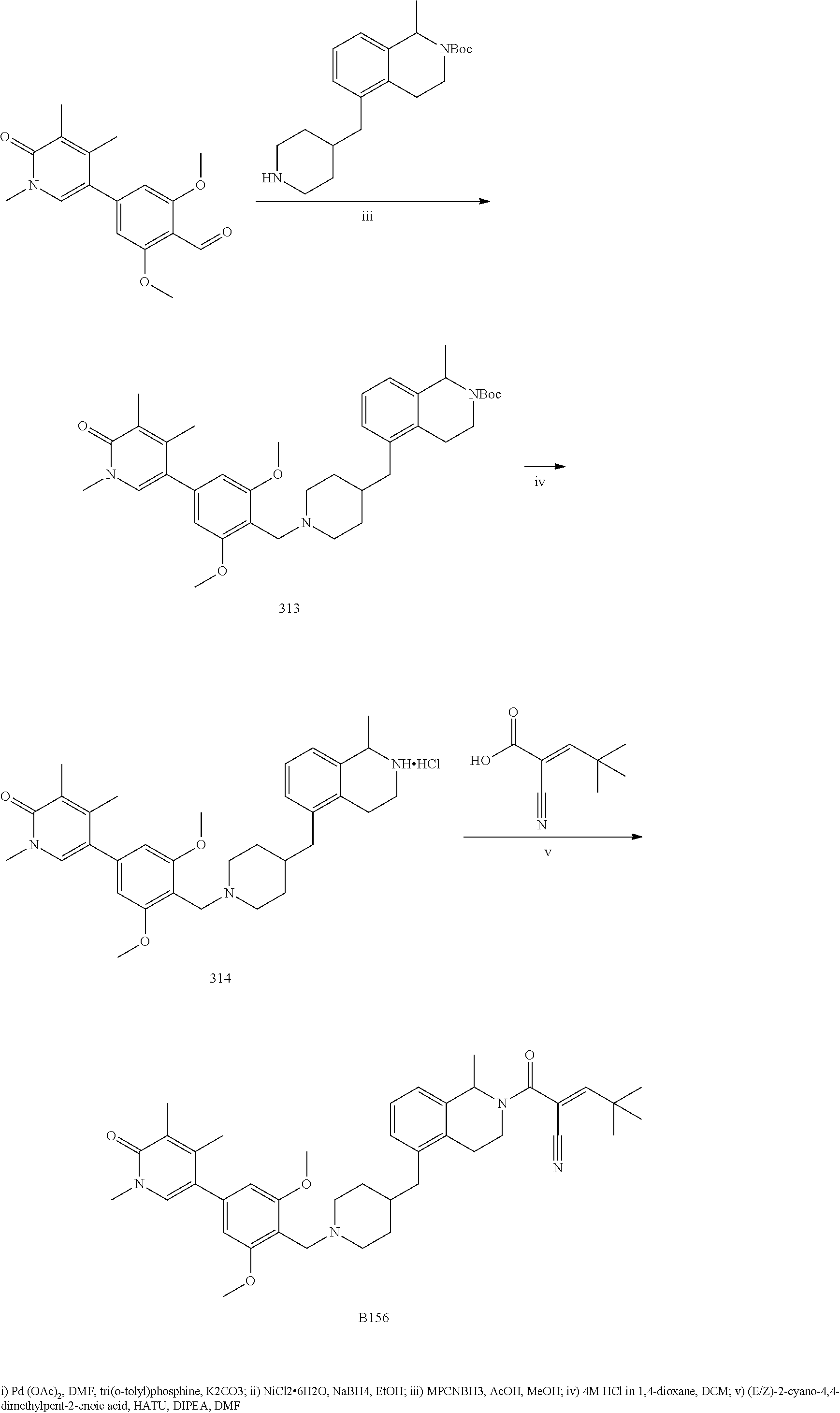

309: To a stirred solution of tert-butyl 5-bromo-1-methyl-3,4-dihydroisoquinoline-2(1H)-carboxylate (564 mg, 1.72 mmol, 1 eq) in DMF (6 mL) was added benzyl 4-methylenepiperidine-1-carboxylate (as prepared in WO2008/84300, 400 mg, 1.72 mmol, 1 eq) and K₂CO₃ (498 mg, 5.18 mmol, 3 eq) at RT. The reaction mixture was degassed with N2 for 10 min, then tri(o-tolyl)phosphine (52.5 mg, 0.17 mmol, 0.5 eq) and palladium acetate (158 mg, 0.17 mmol, 0.1 eq) were added at RT. The reaction mixture was stirred for 16 h at 100° C. before being filtered through celite and concentrated in vacuo. The resulting residue was purified by silica gel column chromatography (gradient=50% EtOAc in petroleum ether) to obtain tert-butyl 5-((1-((benzyloxy)carbonyl)piperidin-4-ylidene)methyl)-1-methyl-3,4-dihydroisoquinoline-2(1H)-carboxylate (290 mg. 0.59 mmol, 34% yield). LCMS m/z=377 [M-100]⁺.

311: To a mixture of tert-butyl 5-((1-((benzyloxy)carbonyl)piperidin-4-ylidene)methyl)-1-methyl-3,4-dihydroisoquinoline-2(1H)-carboxylate (480 mg, 1.0 mmol, 1 eq) in EtOH (10 mL) was added NiCl₂·6H₂O (120 mg, 0.50 mmol, 0.5 eq) at room temperature, followed by NaBH₄ (152 mg, 4.02 mmol, 4 eq) at 0° C. The reaction mixture was stirred overnight at room temperature before being poured in to water and concentrated in vacuo. The aqueous mixture was extracted with ethyl acetate, dried over Na₂SO₄ and concentrated in vacuo. The resulting residue was purified by reverse phase column chromatography to obtain tert-butyl 1-methyl-5-(4-piperidylmethyl)-3,4-dihydro-1H-isoquinoline-2-carboxylate (280 mg, 0.7803 mmol, 77% yield). as off white solid. LCMS m/z=345 [M+H]⁺.

312: By general procedure 14 using MPCNBH₃ (w/w) in DCE:MeOH. Obtained 150 mg, 26.7% yield. LCMS m/z=630.4 [M+H]⁺.

313: By general procedure 2 using 4M HCl in 1,4-dioxane in DCM. Obtained 580 mg (crude). LCMS m/z=530.4 [M+H]⁺.

Compound B156: By general procedure 6 using (E/Z)-2-cyano-4,4-dimethylpent-2-enoic acid (1.5 eq) in DMF. Obtained 15 mg, 0.8% yield. LCMS m/z=665.4 [M+H]⁺.

### (E/Z)-2-(5-((1-(2,6-dimethoxy-4-(1,4,5-trimethyl-6-oxo-1,6-dihydropyridin-3-yl)benzyl)piperidin-4-ylidene)methyl)-1-methyl-1,2,3,4-tetrahydroisoquinoline-2-carbonyl)-4,4-dimethylpent-2-enenitrile (B169)

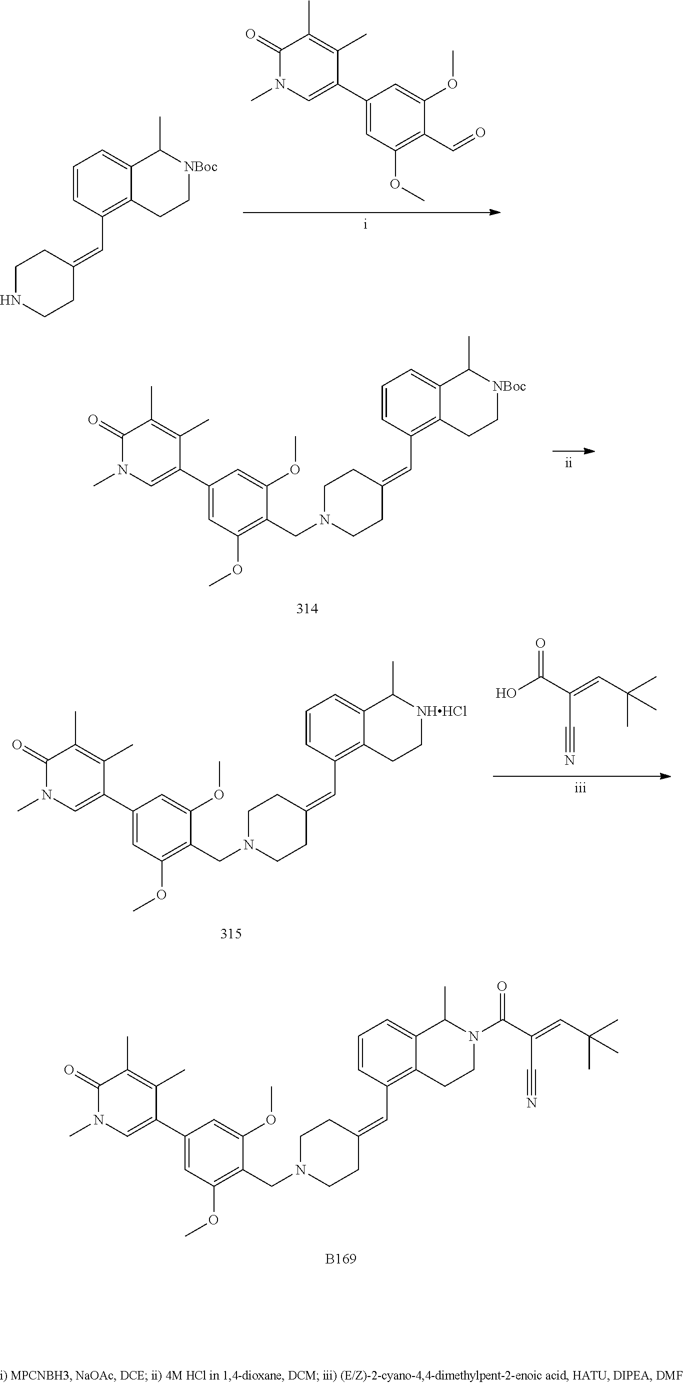

314: By general procedure 14 using MPCNBH₃ (w/w) in DCE: MeOH. Obtained 85 mg, 57.3% yield. LCMS m/z=628.4 [M+H]⁺.

315: By general procedure 2 using 4M HCl in 1,4-dioxane in DCM. Obtained 80 mg, 99% yield). LCMS m/z=528.4 [M+H]⁺.

B169: By general procedure 6 using (E/Z)-2-cyano-4,4-dimethylpent-2-enoic acid (1.5 eq) in DMF. Obtained 7 mg, 6.9% yield. LCMS m/z=663.4 [M+H]⁺.

### (E/Z)-2-(5-((1-(2,6-dimethoxy-4-(1,4,5-trimethyl-6-oxo-1,6-dihydropyridin-3-yl)benzyl)-4-hydroxypiperidin-4-yl)methyl)-1-methyl-1,2,3,4-tetrahydroisoquinoline-2-carbonyl)-4,4-dimethylpent-2-enenitrile (B175)

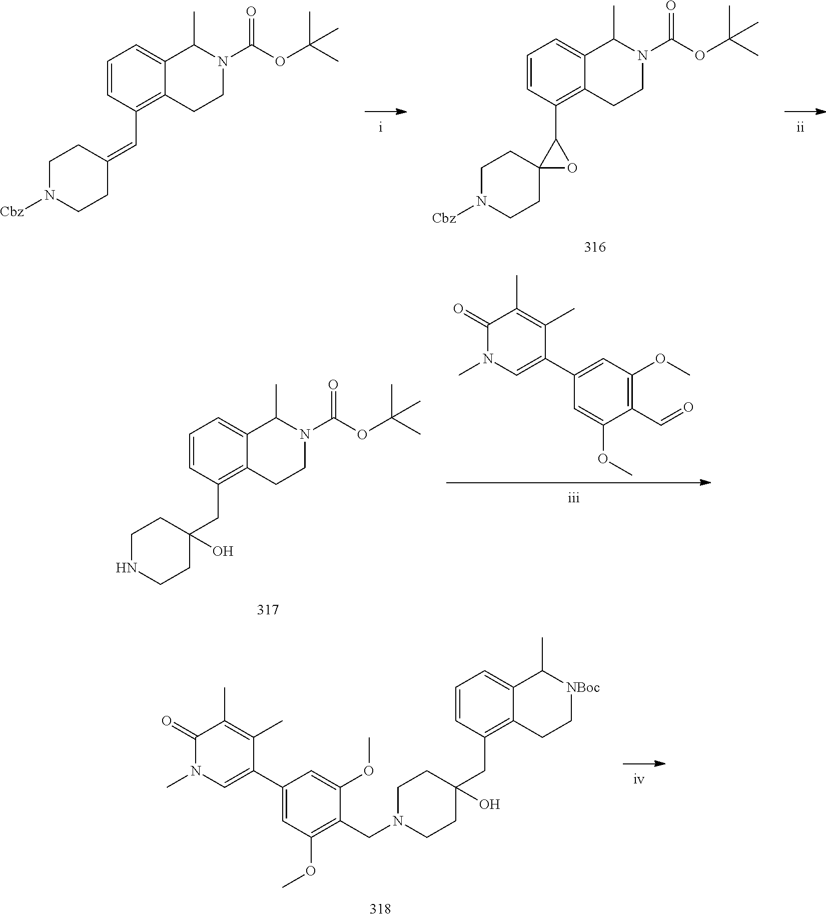

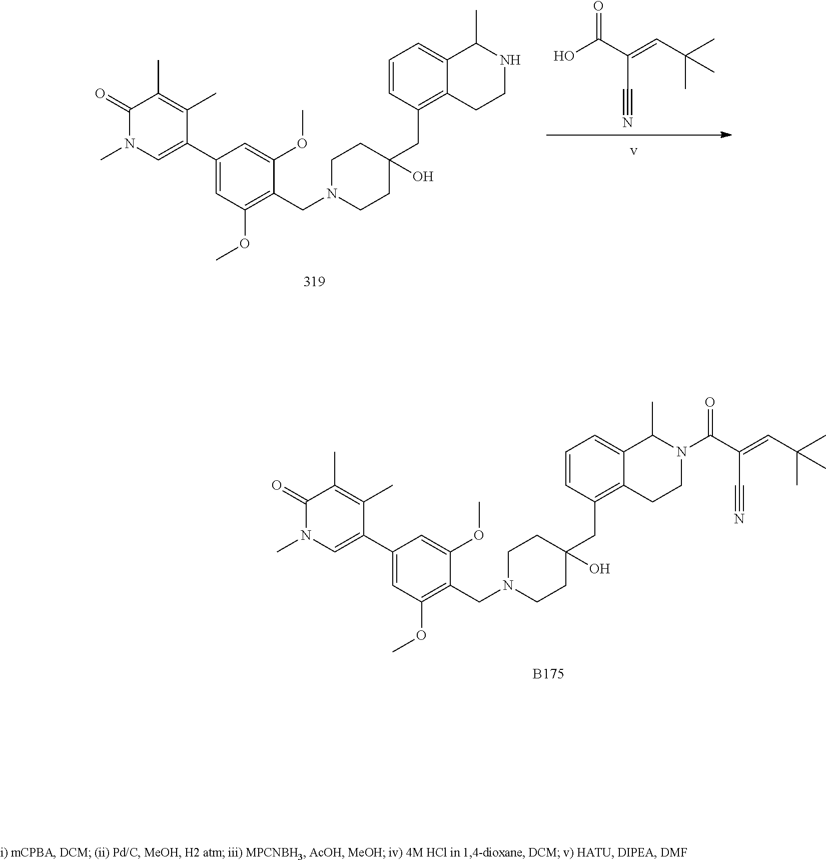 [SMILES: COc1cc(-c2cn(C)c(=O)c(C)c2C)cc(OC)c1CN1CCC(O)(Cc2cccc3c2CCNC3C)CC1 ; CC(C)(C)/C=C(\C#N)C(=O)O ; "319" ; "B175" ; COc1cc(-c2cn(C)c(=O)c(C)c2C)cc(OC)c1CN1CCC(O)(Cc2cccc3c2CCN(C(=O)/C(C#N)=C/C(C)(C)C)C3C)CC1]

316: To a stirred solution of tert-butyl 5-[(1-benzyloxycarbonyl-4-piperidylidene)methyl]-1-methyl-3,4-dihydro-1H-isoquinoline-2-carboxylate (500 mg, 1.04 mmol, 1 eq) in DCM (5 mL) was added 3-chloroperoxybenzoic acid (362 mg, 2.09 mmol, 2 eq) at room temperature and the reaction was stirred for 16 h. The reaction mixture was poured into water and extracted with DCM. The combined organic layers were dried over Na₂SO₄, filtered and concentrated in vacuo to afford benzyl 2-(2-tert-butoxycarbonyl-1-methyl-3,4-dihydro-1H-isoquinolin-5-yl)-1-oxa-6-azaspiro[2.5]octane-6-carboxylate (480 mg, 59% yield). LCMS m/z=393 [M+H]⁺.

317: By general procedure 27 using pd/C. Obtained (310 mg, 76.3% yield. LCMS m/z=361.4 [M+H]⁺. 318: By general procedure 1 using MPCNBH₃ (w/w) in MeOH. Obtained 156 mg, 25.3% yield. LCMS m/z=648.4 [M+H]⁺.

319: By general procedure 2. Obtained 260 mg, 92% yield. LCMS m/z=546.2 [M+H]⁺.

Compound 175: By general procedure 6 using (E/Z)-2-cyano-4,4-dimethylpent-2-enoic acid Obtained (26 mg, 10.6% yield. LCMS m/z=681.4 [M+H]⁺.

### 2-(2-(3-(7-(2,6-dimethoxy-4-(1,4,5-trimethyl-6-oxo-1,6-dihydropyridin-3-yl)benzyl)-2,7-diazaspiro[3.5]nonan-2-yl)phenyl)pyrrolidine-1-carbonyl)-4,4-dimethylpent-2-enenitrile (B43)

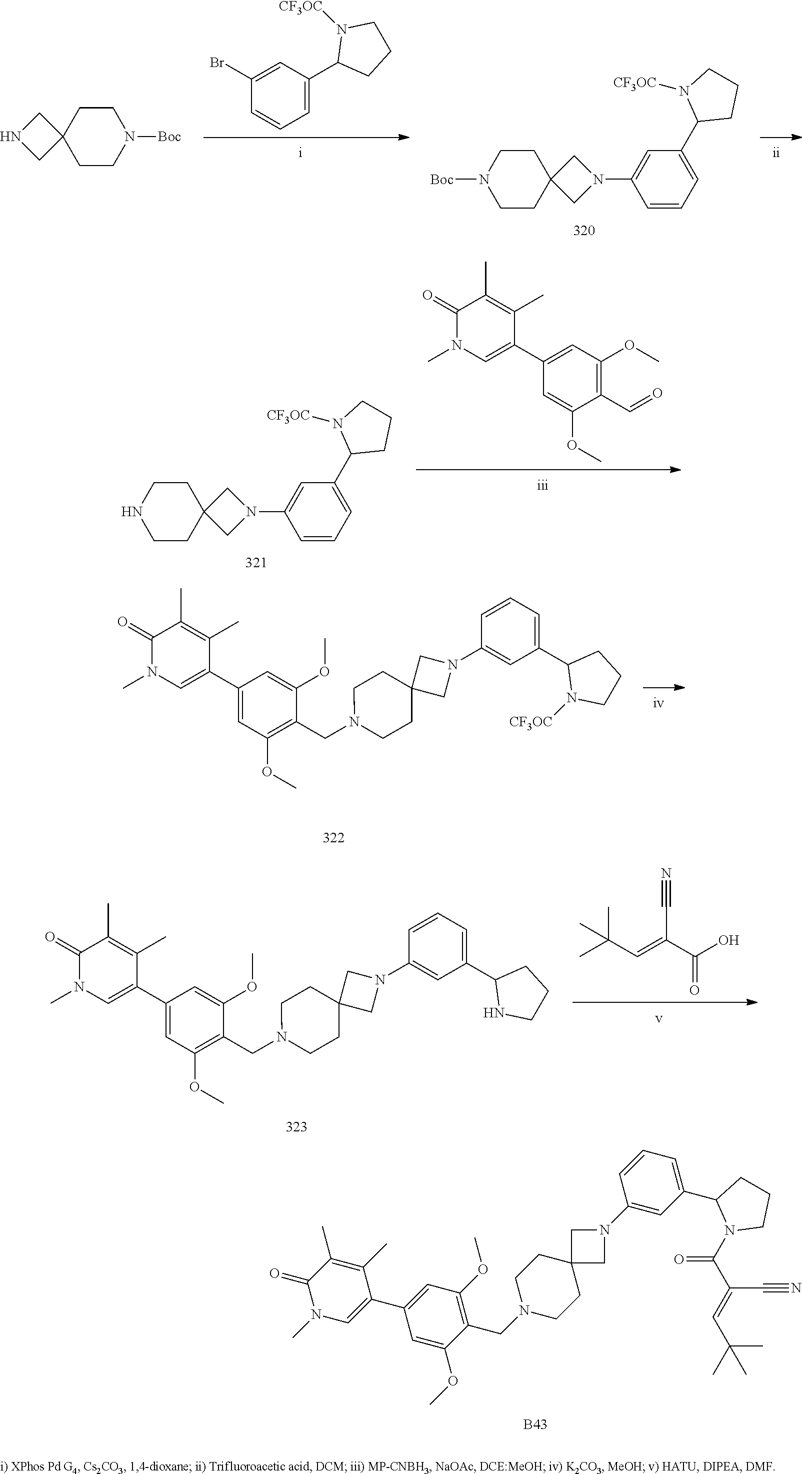

320: By general procedure 21 using tert-butyl 2,7-diazaspiro[3.5]nonane-7-carboxylate (1 eq), XPhos Pd G4 (0.1 eq) and Cs₂CO₃ (3 eq) in 1,4-dioxane. Obtained 1.1 g, 50% yield. LCMS m/z=412.2 [M+H−56]⁺.

321: By general procedure 2. Obtained 1 g (crude). LCMS m/z=368.2 [M+H]⁺.

322: By general procedure 14b. Obtained 2 g, 88% yield. LCMS m/z=653.2 [M+H]⁺.

323: By general procedure 20. Obtained 500 mg (crude). LCMS m/z=517.2 [M+H]⁺.

Compound B43: By general procedure 6 using 2-cyano-4,4-dimethylpent-2-enoic acid (2 eq) in DMF. Obtained 230 mg, 69.6% yield. LCMS m/z=692.3 [M+H]⁺.

### (E/Z)-2-(2-(3-((1-(2,6-dimethoxy-4-(1,4,5-trimethyl-6-oxo-1,6-dihydropyridin-3-yl)benzyl)piperidin-4-yl)oxy)phenyl)pyrrolidine-1-carbonyl)-4,4-dimethylpent-2-enenitrile (B48)

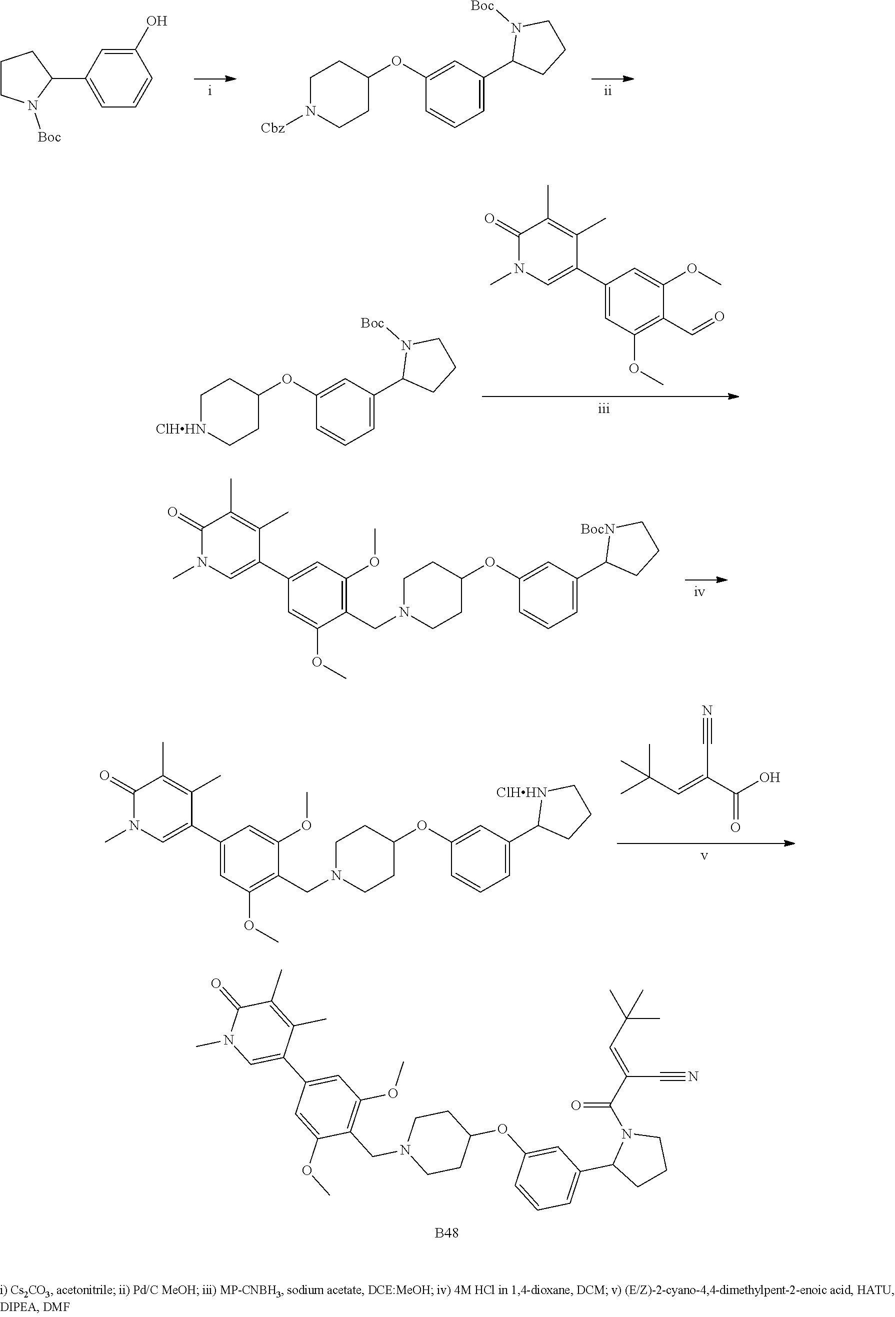

324: By general procedure 26 using Cs₂CO₃ (3 eq) in MeCN. Obtained 770 mg, 47% yield. LCMS m/z=381.4 [M-Boc]r.

325: By general procedure 27. Obtained 120 mg, 27% yield. LCMS m/z=347.2 [M+H]⁺.

326: By general procedure 14 using MPCNBH₃ (w/w). Obtained 110 mg, 52% yield. LCMS m/z=632.4 [M+H]⁺.

327: By general procedure 2. Obtained 92 mg, crude. LCMS m/z=532.6 [M+H].

Compound B48: By general procedure 6 using (E/Z)-2-cyano-4,4-dimethylpent-2-enoic acid (2 eq) in DMF. Obtained 23 mg, 18% yield. LCMS m/z=667.6 [M+H]⁺

### (E/Z)-2-(5-((7-(2,6-dimethoxy-4-(6-methyl-7-oxo-6,7-dihydro-1H-pyrazolo[3,4-c]pyridin-4-yl)benzyl)-4,7-diazaspiro[2.5]octan-4-yl)methyl)-1-methyl-1,2,3,4-tetrahydroisoquinoline-2-carbonyl)-4,4-dimethylpent-2-enenitrile (B38)

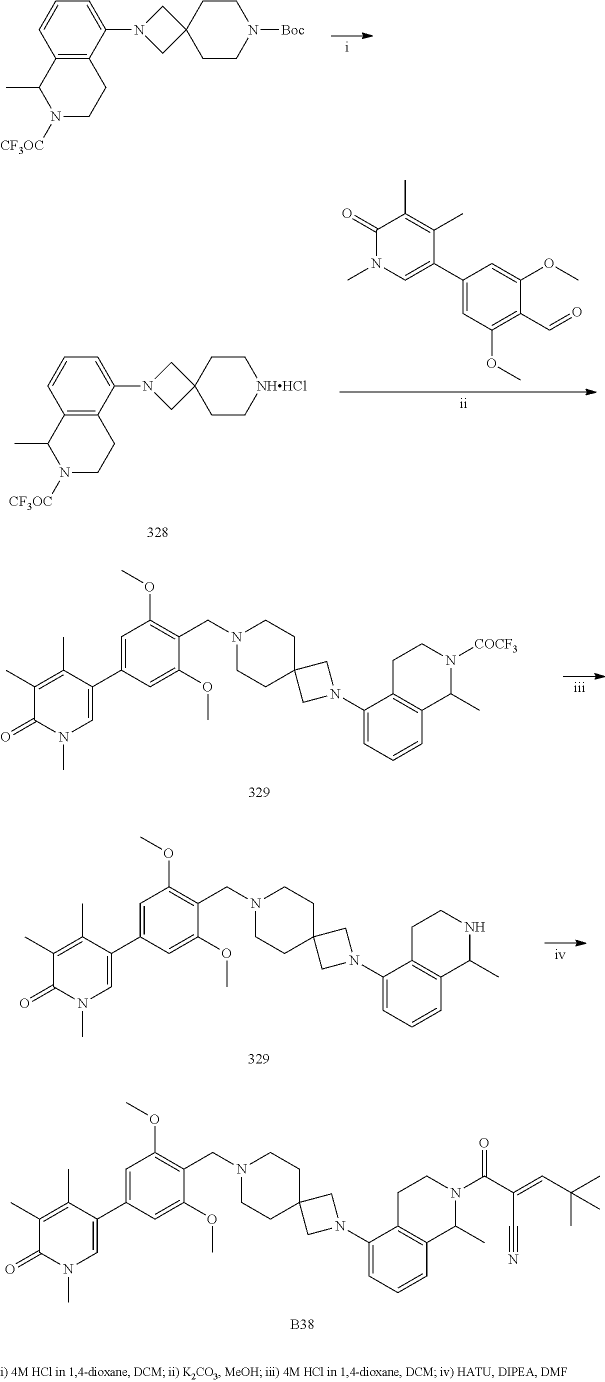

328: By general procedure 2 using 4M HCl in 1,4-dioxane in DCM. Obtained 1.4 g (crude). LCMS m/z=367.9 [M+H]⁺.

329: By general procedure 14 using MP-CNBH₃ (w/w) in DCE:MeOH. Obtained 1.2 g, 55% yield. LCMS m/z=653.2 [M+H]⁺.

330: By general procedure 20 using K₂CO₃ (5 eq) in MeOH. Obtained 400 mg, 48% yield. LCMS m/z=692.4 [M+H]⁺.

Compound B38: By general procedure 6 using (E/Z)-2-cyano-4,4-dimethylpent-2-enoic acid (2 eq) in DMF. Obtained 125 mg, 98.7% yield. LCMS m/z=892.4 [M+H]⁺.

### (E2Z)-2-(5-(7-(2,6-dimethoxy-4-(1,4,5-trimethyl-6-oxo-1,6-dihydropyridin-3-yl)benzyl)-2,7-diazaspiro[4.4]nonan-2-yl)-1-methyl-1,2,3,4-tetrahydroisoquinolin-2-carbonyl)-4,4-dimethylpent-2-enenitrile (C42)

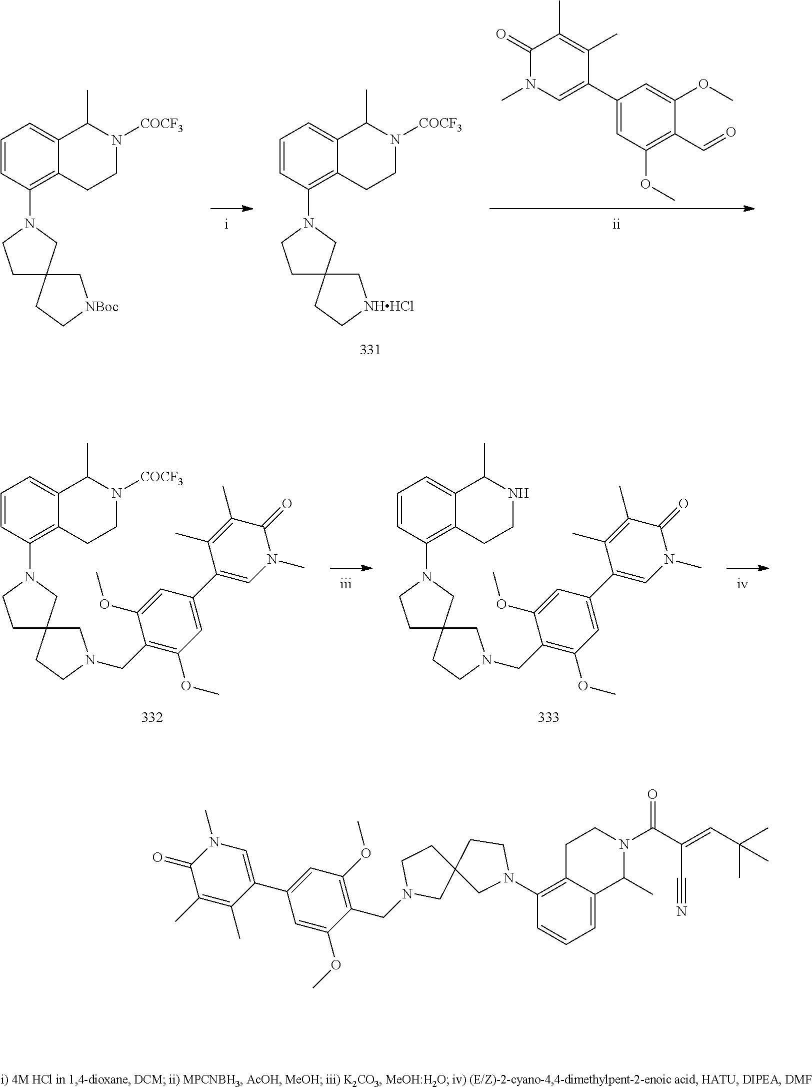

331: By general procedure 2 using 4M HCl in 1,4-dioxane in DCM. Obtained 500 mg (crude). LCMS m/z=388.2 [M+H]⁺.

332: By general procedure 14 using MPCNBH₃ (w/w) in DCE:MeOH. Obtained 400 mg, 49.5% yield. LCMS m/z=653.3 [M+H]⁺.

333: By general procedure 2 using potassium carbonate (2.5 eq) in MeOH:water (4:1). Obtained 0.3 g, 97% yield. LCMS m/z=557.4 [M+H]⁺.

Compound C42: By general procedure 6 using (E/Z)-2-cyano-4,4-dimethylpent-2-enoic acid (1.5 eq) in DMF. Obtained 16.5 mg, 6.67% yield. LCMS m/z=692.3 [M+H]⁺.

### (E/Z)-2-(2-(3-(((2,6-dimethoxy-4-(1,4,5-trimethyl-6-oxo-1,6-dihydropyridin-3-ylbenzyl)methyl)amino)methyl)phenyl)pyrrolidine-1-carbonyl)-4,4-dimethylpent-2-enenitrile (B378)

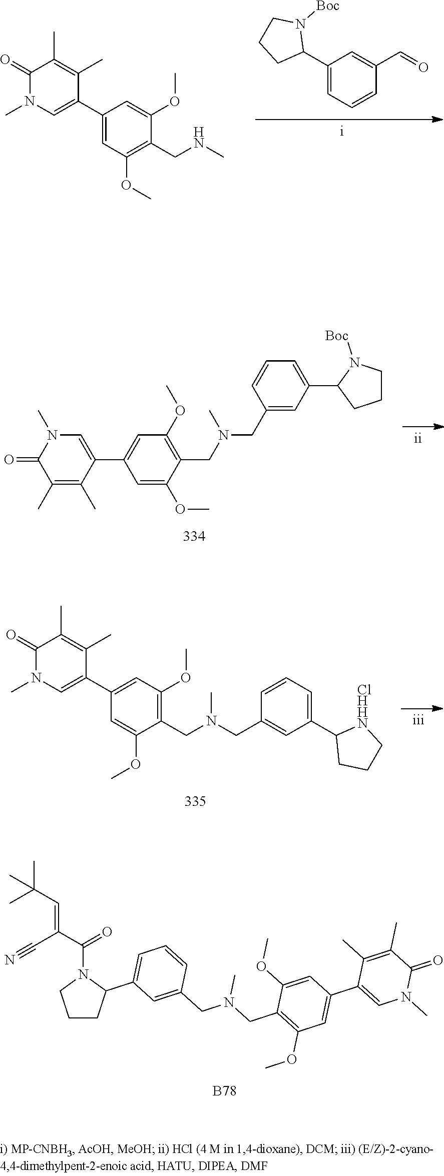

334: By general procedure 14 using MP-CNBH₃ (w/w) in DCE:MeOH. Obtained 400 mg, 43% yield. LCMS m/z=576.3 [M+H]⁺.

335: By general procedure 2. Obtained 320 mg (crude). LCMS m/z=476.2 [M+H]⁺.

Compound B78: By general procedure 6 using (E/Z)-2-cyano-4,4-dimethylpent-2-enoic acid (2 eq) in DMF. Obtained 23 mg, 15% yield. LCMS m/z=611.4 [M+H]⁺.

### Compound C107

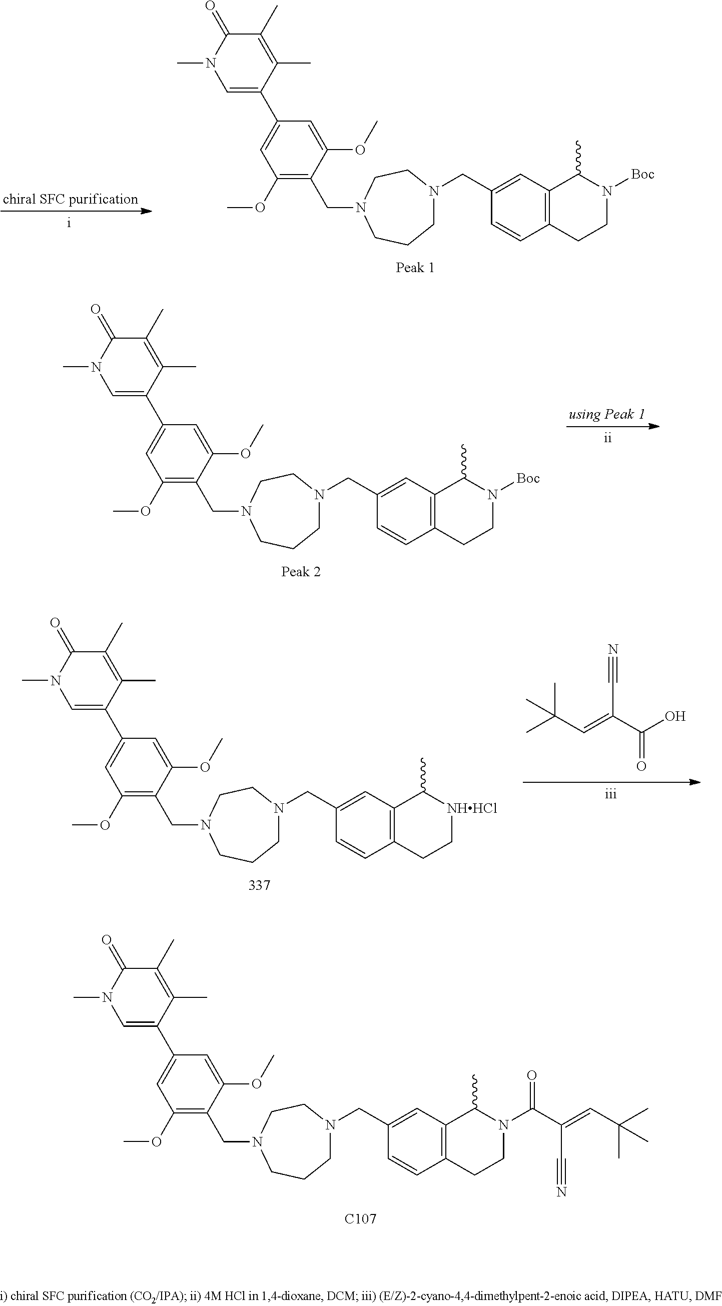

Racemic material (190 mg) was then purified by SFC:

| | |
|---|---|
| Instrument | S/DC/ARD/22-030 |
| Column | WHELK RR-(250*20)mm, 5 μm |
| Mobile Phase | CO₂: 0.5% isopropylamine in MeOH:MeCN [60:40] |
| Flow rate | 50 mL/min |
| Back pressure | 120 bar |
| Wavelength | 254 nm |
| Cycle time | 7 min |

Isolated Peak-1: 75 mg, 41.7% yield. LCMS m/z=645.3 [M+H]⁺ and Peak-2: 70 mg, 38.9% yield. LCMS m/z=645.3 [M+H]⁺

337: Single enantiomer. By general procedure 2 using 4M solution of HCl in 1,4-dioxane, in DCM. Obtained 85 mg (crude). LCMS m/z=545.6 [M+H]⁺.

Compound C107: Single enantiomer. By general procedure 6 using (E/Z)-2-cyano-4,4-dimethylpent-2-enoic acid (2.0 eq) in DMF. Obtained 16.5 mg, 28.9% yield. LCMS m/z=680.4 [M+H]⁺.

### (E/Z)-2-(5-(7-(2,6-dimethoxy-4-(1,4,5-trimethyl-6-oxo-1,6-dihydropyridin-3-yl)benzyl)-4,7-diazaspiro[2.5]octane-4-carbonyl)-1-methyl-1,2,3,4-tetrahydroisoquinoline-2-carbonyl)-4,4-dimethylpent-2-enenitrile (C43)

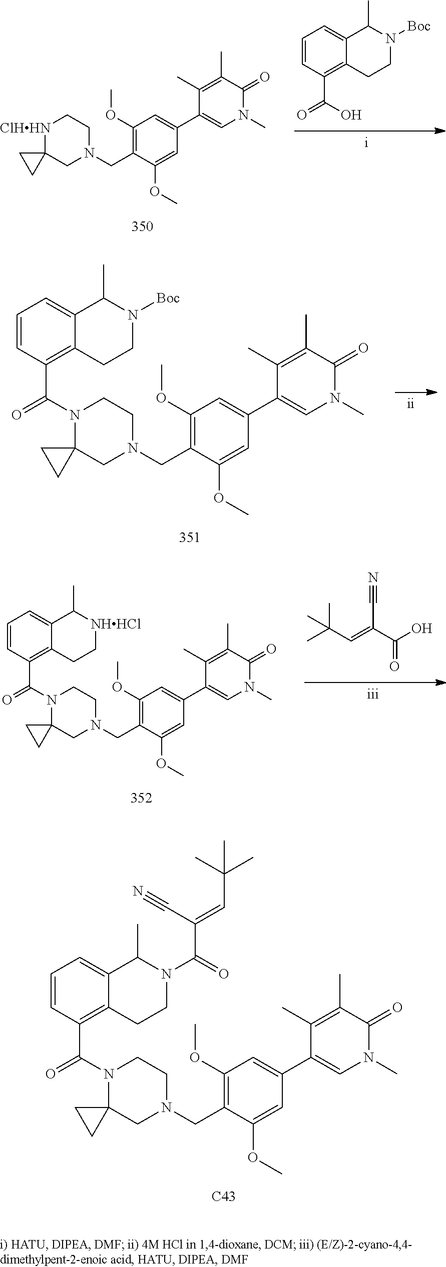

351: By general procedure 6 using commercially available 2-(tert-butoxycarbonyl)-1-methyl-1,2,3,4-tetrahydroisoquinoline-5-carboxylic acid (1 eq) in DMF. Obtained 200 mg, 74% yield. LCMS m/z=671.4 [M+H]⁺.

352: By general procedure 2. Obtained 220 mg (crude). LCMS m/z=571.4 [M+H]⁺.

Compound C43: By general procedure 6 using (E/Z)-2-cyano-4,4-dimethylpent-2-enoic acid (2 eq) in DMF. Obtained 35 mg, 15% yield. LCMS m/z=708.4 [M+H]⁺.

### (E/Z)-2-(7-((1-(2,6-dimethoxy-4-(1,4,5-trimethyl-6-oxo-1,6-dihydropyridin-3-yl)benzyl)azetidin-3-yl(methyl)amino)-1-methyl-1,2,3,4-tetrahydroisoquinoline-2-carbonyl)-4,4-dimethylpent-2-enenitrile (C44)

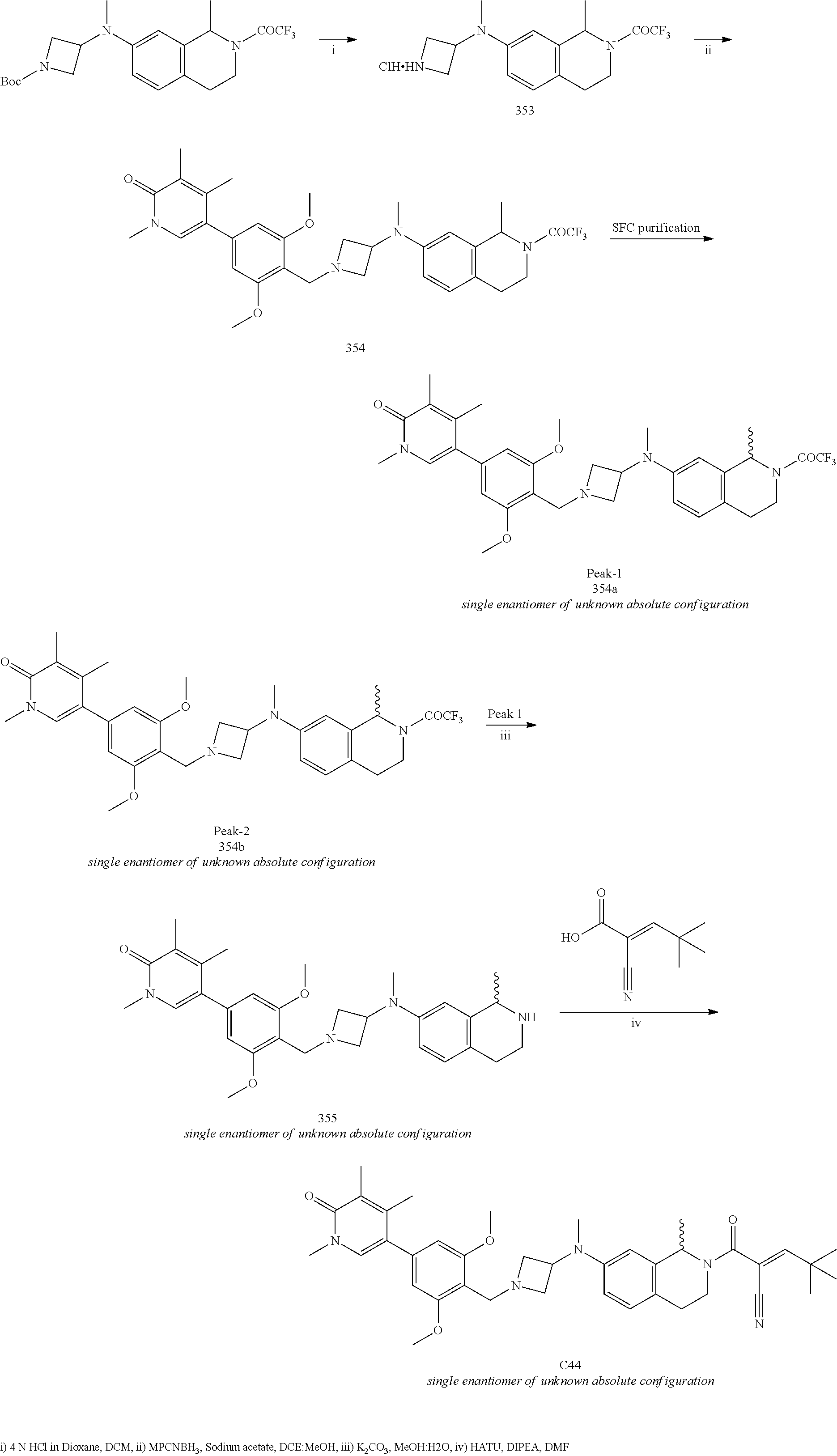

353: By general procedure 2 using HCl in dioxane (4M) in DCM. Obtained (700 mg, 91% yield (crude). LCMS m/z=328.2 (M+H)⁺

354: By general procedure 14 using MP-CNBH₃ (w/w) in DCE:MeOH. Obtained 700 mg, 36.7% yield. LCMS m/z=613.2 [M+H]⁺.

Racemic 364(700 mg) was purified by chiral SFC:

| | |
|---|---|
| Column | WHELK RR-(250*30)mm, 5 μm |
| Mobile Phase | CO₂: 0.5% isopropylamine in MeOH:MeCN [65:35] |
| Flow rate | 4 mL/min |
| Back pressure | 120 bar |
| Wavelength | 254 nm |
| Cycle time | 6 min |

354a: Isolated Peak-1: 312 mg. LCMS m/z=613.3 [M+H]⁺

355: By general procedure 2 using K₂CO₃ (4 eq) in water:MeOH (1:4). Obtained 150 mg (crude), 95% yield. LCMS m/z=517.4 [M+H]⁺.

Compound C44: By general procedure 6. Obtained 50 mg, 35% yield. LCMS m/z=652.4 [M+H]⁺

### (E/Z)-2-(5-(7-(2,6-dimethoxy-4-(1,4,5-trimethyl-6-oxo-1,6-dihydropyridin-3-yl)benzyl)-2,7-diazaspiro[3.5]nonan-2-yl)-1-methyl-1,2,3,4-tetrahydroisoquinoline-2-carbonyl)-4,4-dimethylpent-2-enenitrile (C45)

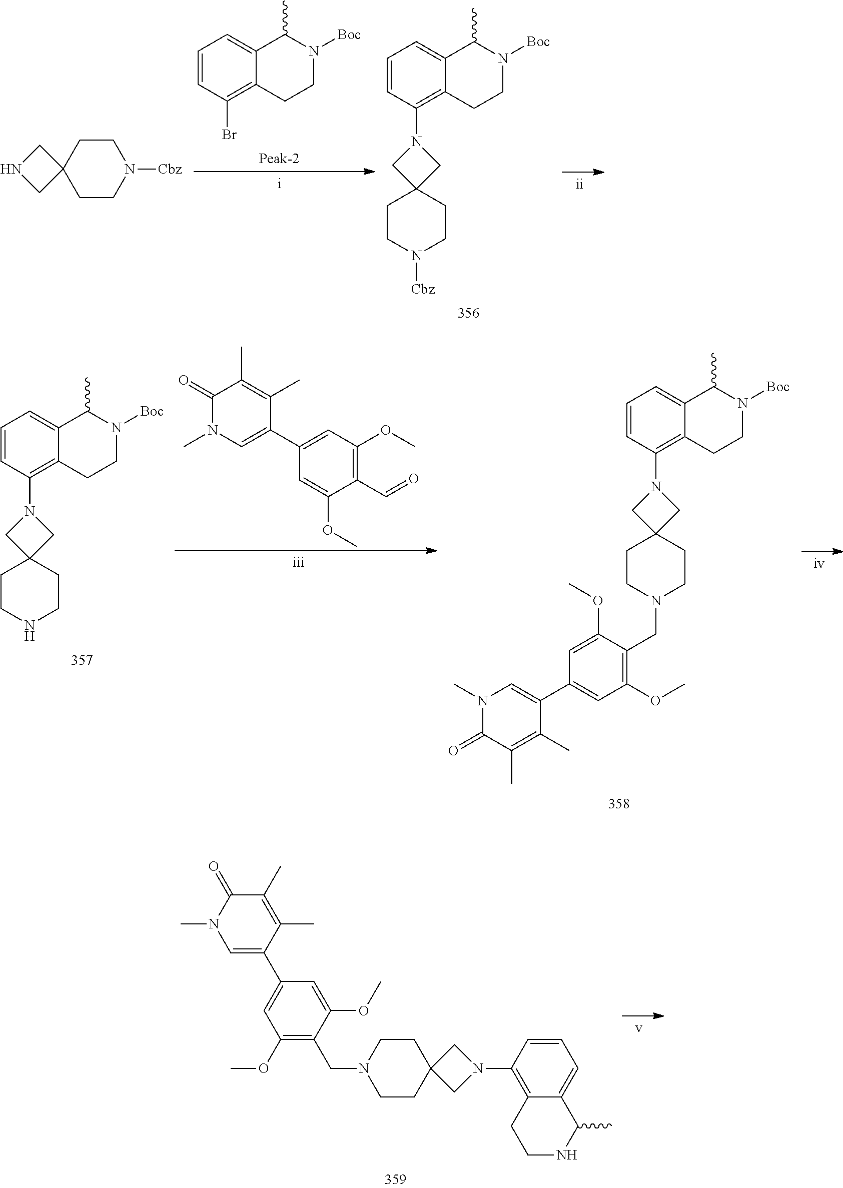

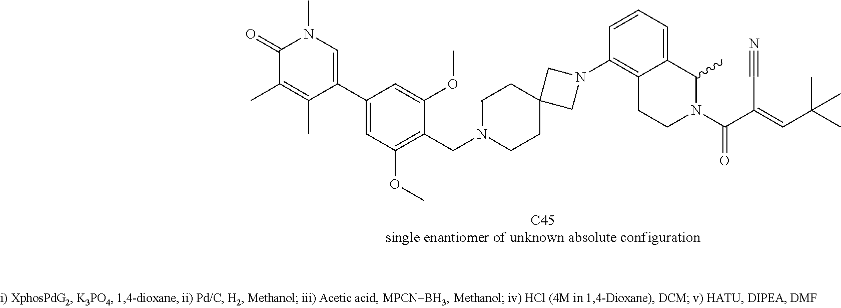 [SMILES: COc1cc(-c2cn(C)c(=O)c(C)c2C)cc(OC)c1CN1CCC2(CC1)CN(c1cccc3c1CCN(C(=O)/C(C#N)=C/C(C)(C)C)C3C)C2]

357: By general procedure 27. Obtained 410 mg, 78.5% yield. LCMS m/z=372.4 [M+H]⁺

358: By general procedure 1 using MP-CNBH₃ (w/w) in MeOH. Obtained 610 mg, 81.2% yield. LCMS m/z 657.4 [M+H]⁺

359: By general procedure 2 using 4M solution of 1,4-dioxane in HCl in DCM. Obtained 510 mg (crude). LCMS m/z=557.4 [M+H]⁺.

Compound C45: By general procedure 6 using (E)-2-cyano-4,4-dimethylpent-2-enoic acid (1.5 eq) in DMF. Obtained 14.2 mg, 6% yield. LCMS m/z=692.3 [M+H]⁺.

### Compound C46

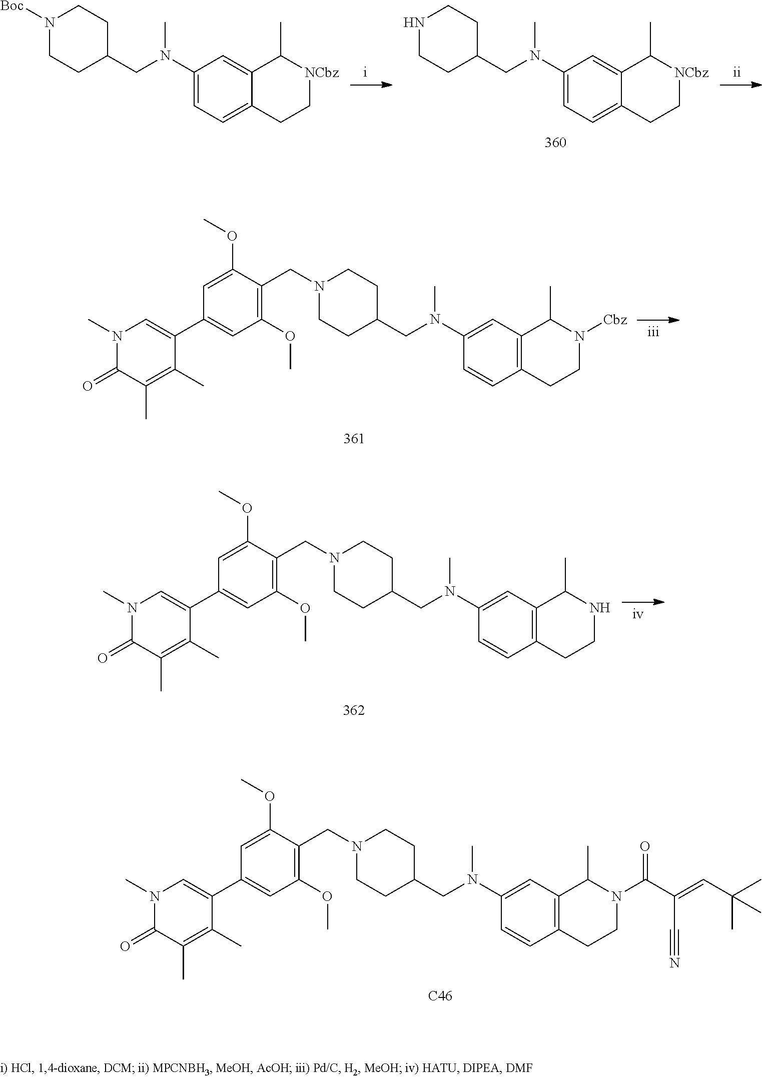 [SMILES: CC1c2cc(N(C)CC3CCN(C(=O)OC(C)(C)C)CC3)ccc2CCN1C(=O)OCc1ccccc1 ; CC1c2cc(N(C)CC3CCNCC3)ccc2CCN1C(=O)OCc1ccccc1 ; COc1cc(-c2cn(C)c(=O)c(C)c2C)cc(OC)c1CN1CCC(CN(C)c2ccc3c(c2)C(C)N(C(=O)OCc2ccccc2)CC3)CC1 ; COc1cc(-c2cn(C)c(=O)c(C)c2C)cc(OC)c1CN1CCC(CN(C)c2ccc3c(c2)C(C)NCC3)CC1 ; COc1cc(-c2cn(C)c(=O)c(C)c2C)cc(OC)c1CN1CCC(CN(C)c2ccc3c(c2)C(C)N(C(=O)/C(C#N)=C/C(C)(C)C)CC3)CC1 ; "C46"]

360: By general procedure 2 using HCl (4M solution in 1,4-dioxane) in DCM. Obtained 450 mg (crude). LCMS m/z=408.4 [M+H]⁺.

361: By general procedure 1 using MP-CNBH₃ (WAV) in MeOH. Obtained 230 mg, 25% yield. LCMS. m/z=693.3 [M+H]⁺.

362: By general procedure 27. Obtained 150 mg, 90% yield. LCMS m/z=559.4 [M+H]⁺.

Compound C46: By general procedure 6. Obtained 2.8 mg, 5.4% yield. LCMS m/z=694.4 [M+H]⁺.

### Compound C47 and Compound C48 and (E/Z)-2-(5-(8-(2,6-dimethoxy-4-(1,4,5-trimethyl-4-oxo-1,6-dihydropyridin-3-yl)benzyl)-2,8-diazaspiro[4.5]decan-2-yl)-1-methyl-1,2,3,4-tetrahydroisoquinoline-2-carbonyl)-5-fluoro-4,4-dimethylpent-2-enenitrile

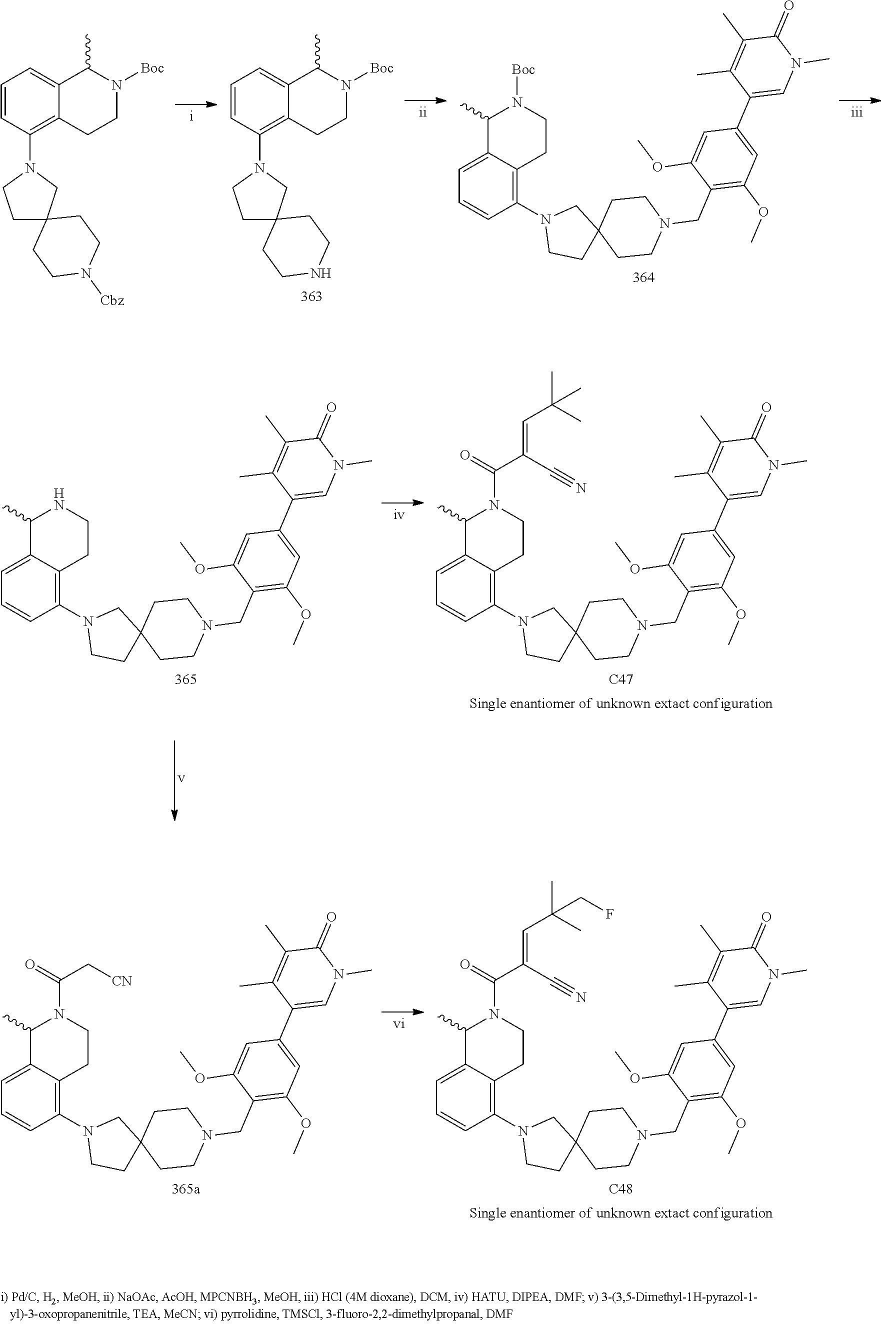

363: By general procedure 27. Obtained 175 mg, 77% yield LCMS m/z=386.3 [M+H]⁺.

364: By general procedure 1 using MP-CNBH₃ (w/w), sodium acetate (2.5 eq) and acetic acid (0.1 eq) in MeOH. Obtained 250 mg, 94% yield. LCMS m/z=671.4 [M+H]⁺.

365: By general procedure 2 using HCl in dioxane (4M) in DCM. Obtained 200 mg, (crude). LCMS m/z=571.2 (M+H)⁺.

Compound C47: By general procedure 6. Obtained 8 mg, 6% yield. LCMS m/z=706.6 [M+H]⁺

365a: By general procedure 3. Obtained 250 mg, 79% yield. LCMS m/z=638.4 [M+H]⁺.

Compound C48: By general procedure 4a. Obtained 26 mg, 38% yield. LCMS m/z=724.3 [M+H]⁺.

### Compound C49

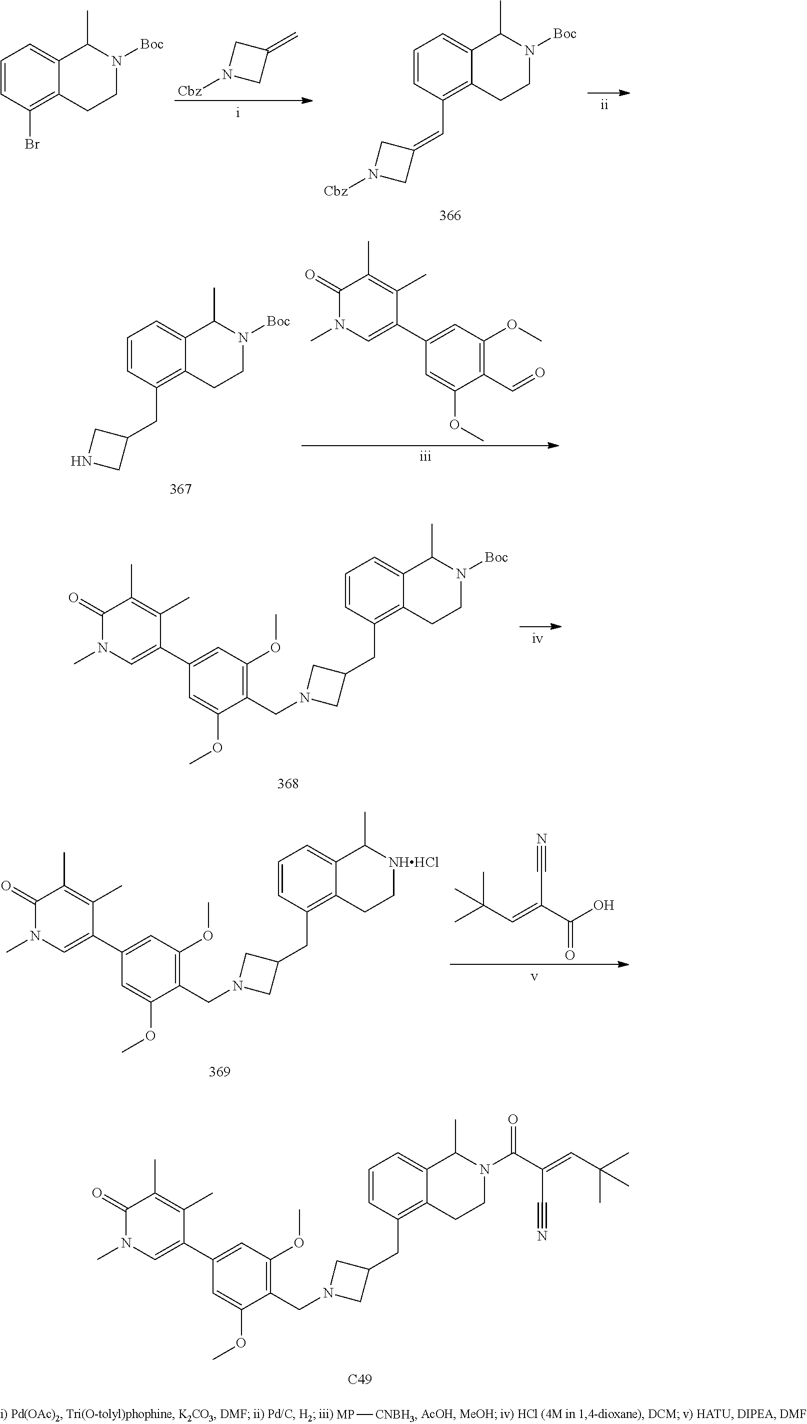

366: By general procedure 31 using starting material as described in WO20138002. Obtained 200 mg, 76% yield. LCMS m/z=349.2 [M+H]⁺.

367: By general procedure 27. Obtained 167 mg (crude). LCMS m/z=317.2 [M+H]⁺.

368: By general procedure 14 using MP-CNBH₃ (w/w) in DCE:MeOH. Obtained 60 mg, 18% yield. LCMS m/z=602.3 [M+H]⁺.

369: By general procedure 2 using 4M solution of HCl in 1,4-dioxane, in DCM. Obtained 45 mg (crude), 75% yield. LCMS m/z=502.2 [M+H]⁺.

Compound C49: By general procedure 6 using (E/Z)-2-cyano-4,4-dimethylpent-2-enoic acid (3 eq) in DMF. Obtained 1.2 mg, 3.9% yield. LCMS m/z=637.3 [M+H]⁺

### Compounds C50 and C51

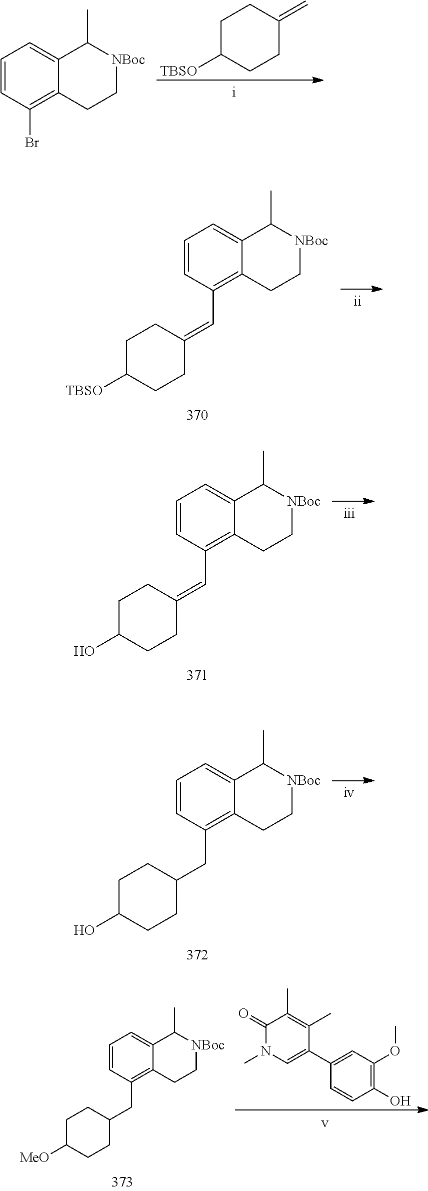

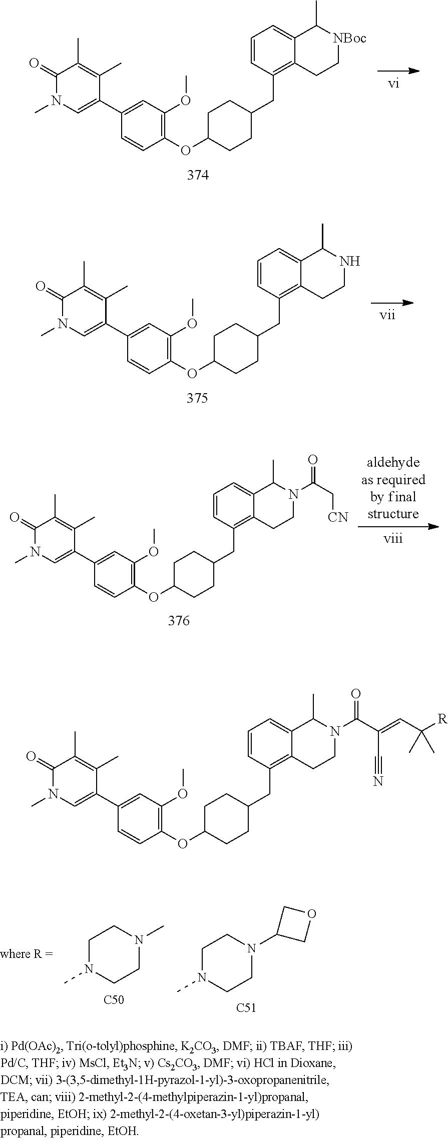

370: By general procedure 31. Obtained 1.1 g, 51% yield. LCMS m/z=471.8 [M-Boc]⁺

371: To a stirred solution of tert-butyl 5-((4-((tert-butyldimethylsilyl)oxy)cyclohexylidene)methyl)-1-methyl-3,4-dihydroisoquinoline-2(1H)-carboxylate (1.1 g, 2.332 mmol, 1 eq) in THF (5 mL) was added TBAF (1M in THF, 4.66 mL, 4.66 mmol, 2 eq) at 0° C. and the reaction mixture was stirred overnight at RT The reaction mixture was concentrated in vacuo and the resulting residue was purified by silica gel column chromatography (gradient=0-20% EtOAc in hexane). The appropriate fractions were concentrated in vacuo to obtain tert-butyl 5-((4-hydroxycyclohexylidene)methyl)-1-methyl-3,4-dihydroisoquinoline-2(1H)-carboxylate 475 mg, 1.315 mmol, 56.4% yield. LCMS m/z=357.49 [M-Boc]⁺.

372: To a stirred solution of tert-butyl 5-((4-hydroxycyclohexylidene)methyl)-1-methyl-3,4-dihydroisoquinoline-2(1H)-carboxylate (475 mg, 1.329 mmol, 1 eq) in THF (6 mL) was added Pd/C (566 mg, 0.531 mmol, 0.4 eq) under nitrogen atmosphere. The reaction mixture was stirred under H₂ overnight then was filtered through celite. The celite was washed with EtOAc and the combined organics phases were concentrated in vacuo to obtain tert-butyl 5-((4-hydroxycyclohexyl)methyl)-1-methyl-3,4-dihydroisoquinoline-2(1H)-carboxylate (430 mg, 89% yield). The compound was used without further purification. LCMS m/z=359.5 [M-Boc]⁺

373: By general procedure 25. Obtained 380 mg, 72.6% yield. The

374: By general procedure 26. Obtained 100 mg, 24.2% yield. LCMS m/z=600.8 [M+H]⁺

375: By general procedure 2 using 4M solution of dioxane in HCl in DCM. Obtained 100 mg, 90% yield. LCMS m/z=500.7 [M+H]⁺.

376: By general procedure 3. Obtained 100 mg, 83% yield. LCMS m/z=567.7 [M+H]⁺.

Compound C50: By general procedure 4 in ethanol. Obtained 30 mg, 29% yield. LCMS m/z=719.9 [M+H]⁺.

Compound C51: By general procedure 4 in ethanol. Obtained 51 mg, 24.8% yield. LCMS m/z=762.0 [M+H]⁺.

### Compound C52

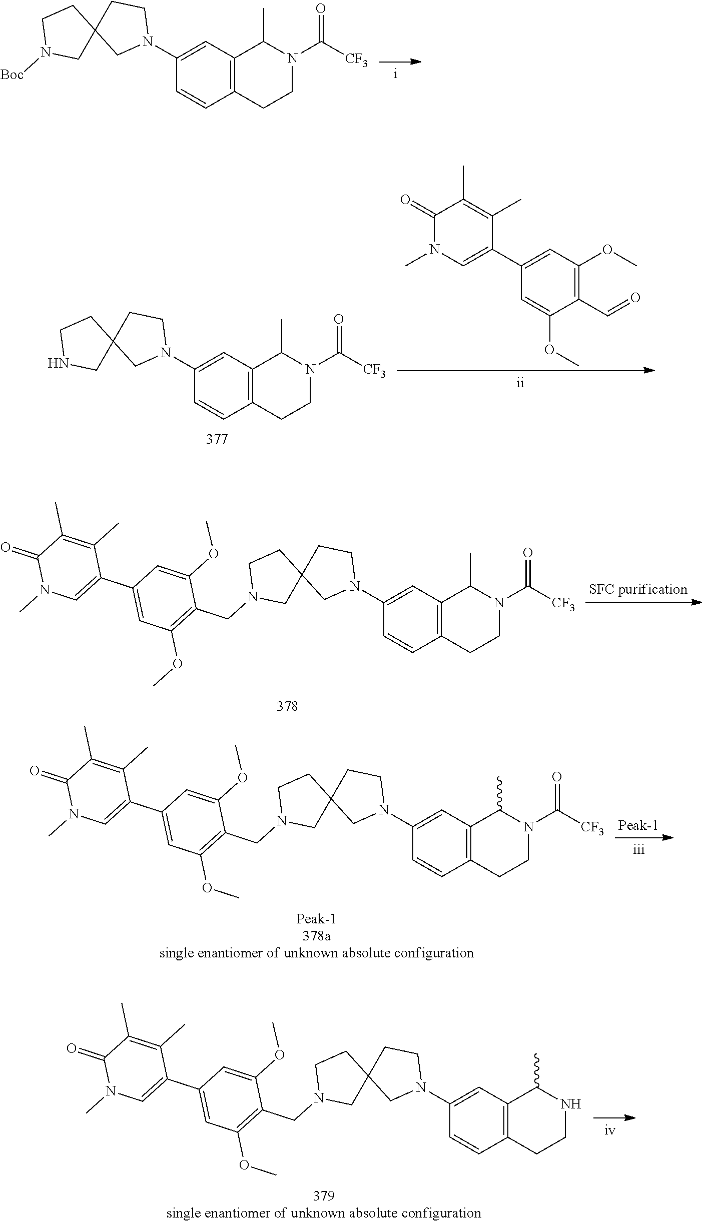

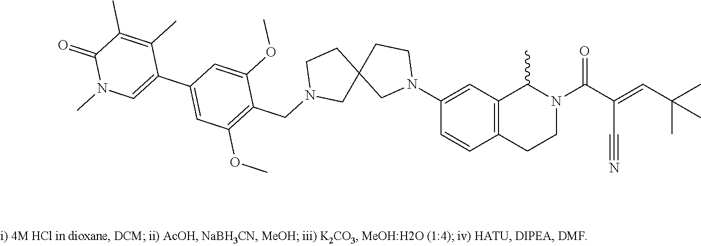 [SMILES: COc1cc(-c2cn(C)c(=O)c(C)c2C)cc(OC)c1CN1CCC2(CCN(c3ccc4c(c3)C(C)N(C(=O)/C(C#N)=C/C(C)(C)C)CC4)C2)C1]

377: By general procedure 2 using HCl (4 M in dioxane), in DCM. Obtained 3.9 g. LCMS m/z=368.2 [M+H]⁺.

378: By general procedure 1 using MP-CNBH₃ (w/w) in MeOH. Obtained 4.24 g. LCMS m/z=653.4 [M+H]⁺

Racemic material (4.22 g) was then purified by SFC:

| | |
|---|---|
| Column | WHELK RR-(250*20)mm, 5 μm |
| Mobile Phase | CO₂: 0.5% isopropylamine in MeOH:MeCN [55:45] |
| Flow rate | 4 mL/min |
| Back pressure | 100 bar |
| Wavelength | 254 nm |
| Cycle time | 7 min |

378a: Isolated Peak-1: 1.65 g, 38.7% yield. LCMS m/z=653.2 [M+H]⁺

379: By general procedure 20. Obtained 290 mg. LCMS m/z=557.6 [M+H]⁺.

Compound C62: By general procedure 6 in DMF. Obtained 55 mg, 29% yield. LCMS m/z=692.6 [M+H]⁺

### Compound C53

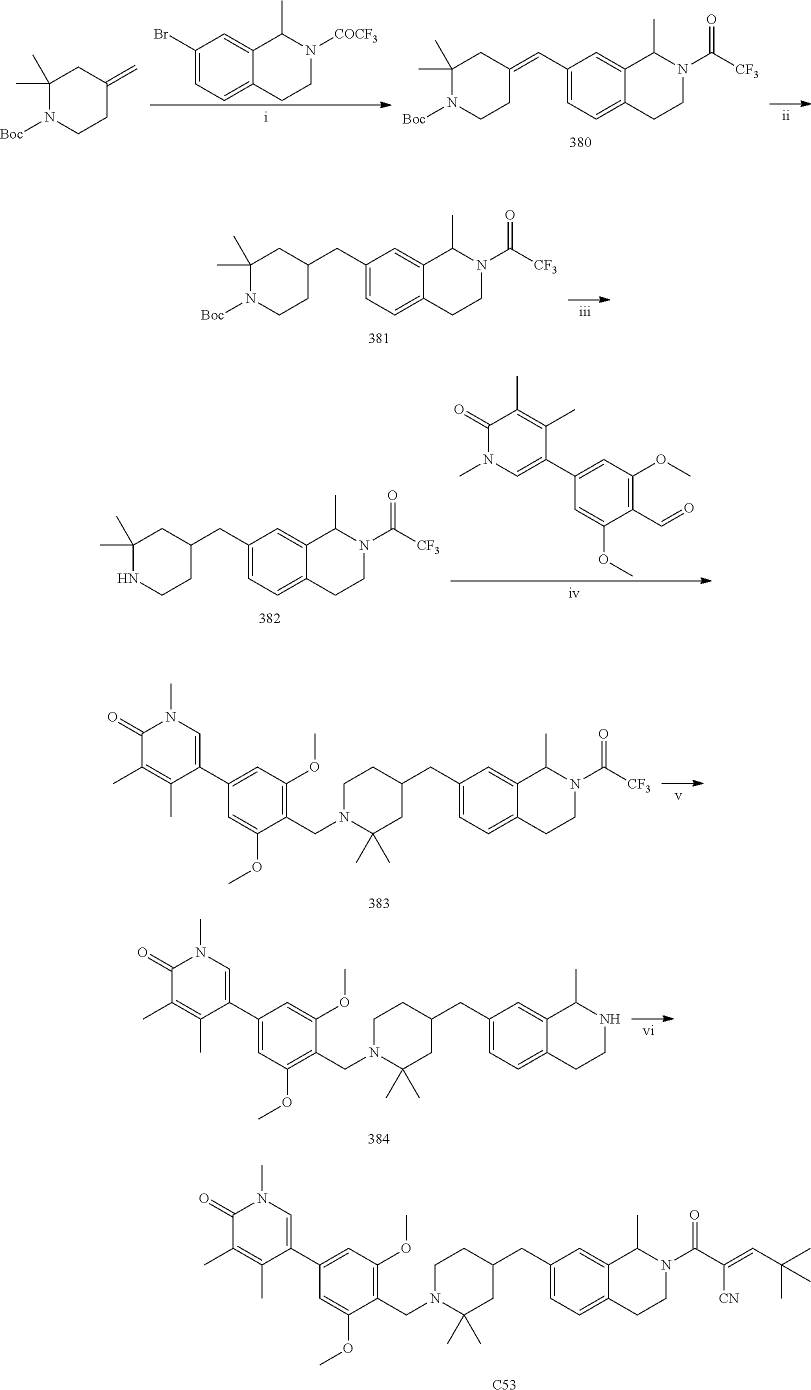

380: By general procedure 31 using material as described in WO2020/96916. Obtained 762 mg, 65% yield. LCMS m/z=376.3 [M-Boc].

381: To a stirred solution of tert-butyl (E/Z)-2,2-dimethyl-4-((1-methyl-2-(2,2,2-trifluoroacetyl)-1,2,3,4-tetrahydroisoquinolin-7-yl)methylene)piperidine-1-carboxylate (750 mg, 1.60 mmol, 1 eq) in EtOAc (6 mL) was added Pd/C (513 mg, 4.82 mmol, 3 eq). The reaction mixture was stirred under H₂ overnight then was filtered through celite. The celite was washed with DCM and MeOH and the combined organics phases were concentrated in vacuo to obtain tert-butyl 2,2-dimethyl-4-((1-methyl-2-(2,2,2-trifluoroacetyl)-1,2,3,4-tetrahydroisoquinolin-7-yl)methyl)piperidine-1-carboxylate (750 mg, 1.585 mmol, 99% yield). LCMS m/z=369.3 [M-Boc+H]⁺,

382: By general procedure 2 using HCl (4 M In 1,4-dioxane) in DCM. Obtained 580 mg. LCMS m/z=369.2 [M+H]⁺

383: By general procedure 14 using MP-CNBH₃ (w/w) in MeOH. Obtained 492 mg, 49% yield. LCMS m/z=654.3 [M+H]⁺

384: By general procedure 20. Obtained 410 mg. (crude). LCMS m/z=558.4 [M+H]⁺

Compound C53: By general procedure 6 in DMF. Obtained 40 mg, 10% yield. LCMS m/z=693.4 [M+H]⁺

### Compound: Compound C64

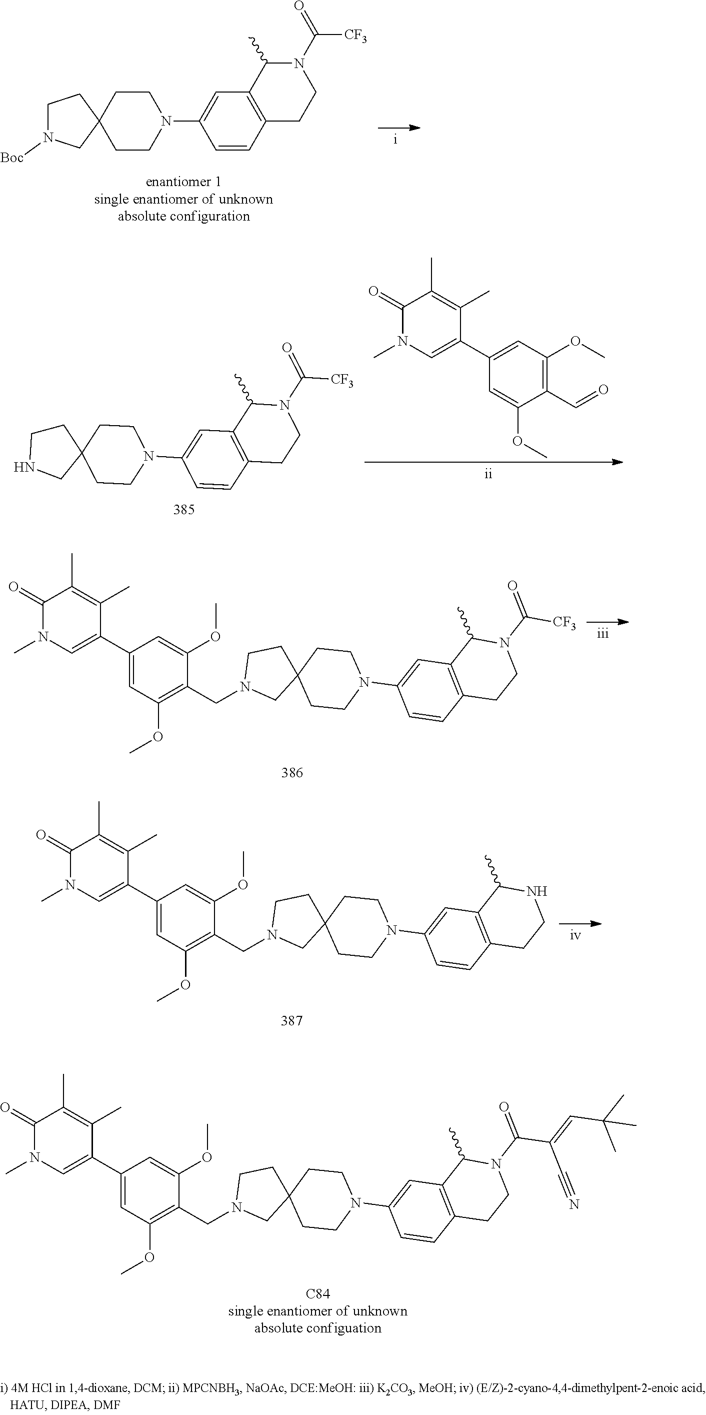

385 By general procedure 2 using dioxane in HCl (4M in 1,4-dioxane) in DCM. Obtained 0.8 g, 88% yield.

386: By general procedure 14 using MPCNBH₃ (w/w) in DCE: MeOH. Obtained 1.2 g, 93% yield. Obtained 1.6 g (crude). LCMS m/z=687.8 [M+H]⁺.

387: By general procedure 20. Obtained 0.34 g, 88% yield. LCMS m/z=571.4 [M+H]⁺.

Compound C54: By general procedure 6. Obtained 120 mg, 31% yield. LCMS m/z=706.2 [M+H]⁺.

### Compound C55

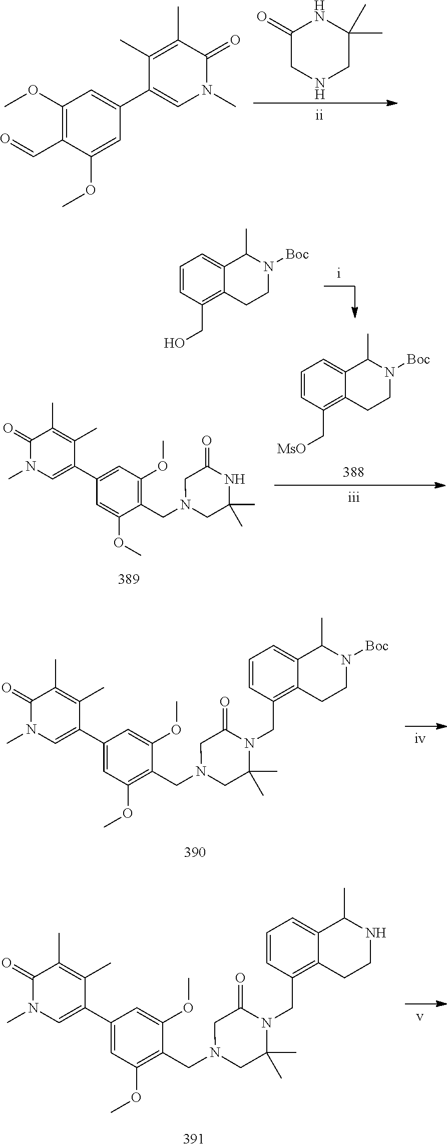

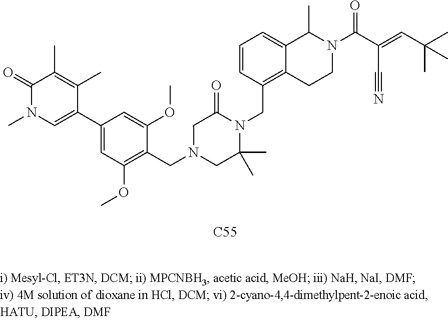 [SMILES: COc1cc(-c2cn(C)c(=O)c(C)c2C)cc(OC)c1CN1CC(=O)N(Cc2cccc3c2CCN(C(=O)/C(C#N)=C/C(C)(C)C)C3C)C(C)(C)C1 ; "C55"]

388: To a stirred solution of tert-butyl 5-(hydroxymethyl)-1-methyl-3,4-dihydroisoquinoline-2(1H)-carboxylate (300 mg, 1.082 mmol, 1 eq) in DCM (5 mL) at 0° C. was added Et3N (0.376 ml, 2.70 mmol, 2.5 eq) followed by the addition of mesyl-CI (0.126 ml, 1.622 mmol, 1.5 eq). The reaction was stirred at RT for 2 h before being quenched with water and extracted with DCM. The organic layer was concentrated in vacuo and the resulting residue was purified silica gel column chromatography (gradient=EtOAc in hexane 0-30%). The appropriate fractions were concentrated in vacuo to give tert-butyl 1-methyl-5-(((methylsulfonyl)oxy)methyl)-3,4-dihydroisoquinoline-2(1H)-carboxylate (250 mg. 0.703 mmol, 65% yield). LCMS m/z=240.2 [M−56]⁺.

389: By general procedure 1 using MPCNBH₃ (w/w) in MeOH. Obtained 350 mg, 43% yield. LCMS m/z=414.4 [M+H]⁺.

390: To a stirred solution of 4-(2,6-dimethoxy-4-(1,4,5-trimethyl-6-oxo-1,6-dihydropyridin-3-yl)benzyl)-6,6-dimethylpiperazin-2-one (100 mg, 0.242 mmol, 1 eq) in DMF (1 mL) was added NaH (21.10 mg, 0.484 mmol, 2 eq) at RT and the reaction was stirred for 5 min. Tert-butyl 5-(chloromethyl)-1-methyl-3,4-dihydroisoquinoline-2(1H)-carboxylate (107 mg, 0.363 mmol, 1.5 eq), and NaI (36.2 mg, 0.242 mmol, 2 eq) were added and the reaction mixture was stirred at 80° C. for 16 h. The reaction mixture was quenched with ice water and was extracted with DCM. The combined organic layers were dried over anhydrous sodium sulphate filtered and concentrated in vacuo. The resulting residue was purified by silica gel column chromatography (gradient=0-10% MeOH in DCM). The appropriate fractions were concentrated in vacuo to afford tert-butyl 5-((4-(2,6-dimethoxy-4-(1,4,5-trimethyl-8-oxo-1,6-dihydropyridin-3-yl)benzyl)-2,2-dimethyl-6-oxopiperazin-1-yl)methyl)-1-methyl-3,4dihydroisoquinoline-2(1H)-carboxylate (80 mg, 45.1% yield). LCMS m/z=673.4 [M+H]⁺.

391: By general procedure 2. Obtained 70 mg, crude. LCMS m/z=573.2 [M+H]⁺.

Compound C55: By general procedure S. Obtained 15 mg, 21% yield. LCMS m/z=708.4 [M+H]⁺.

### Compound C56

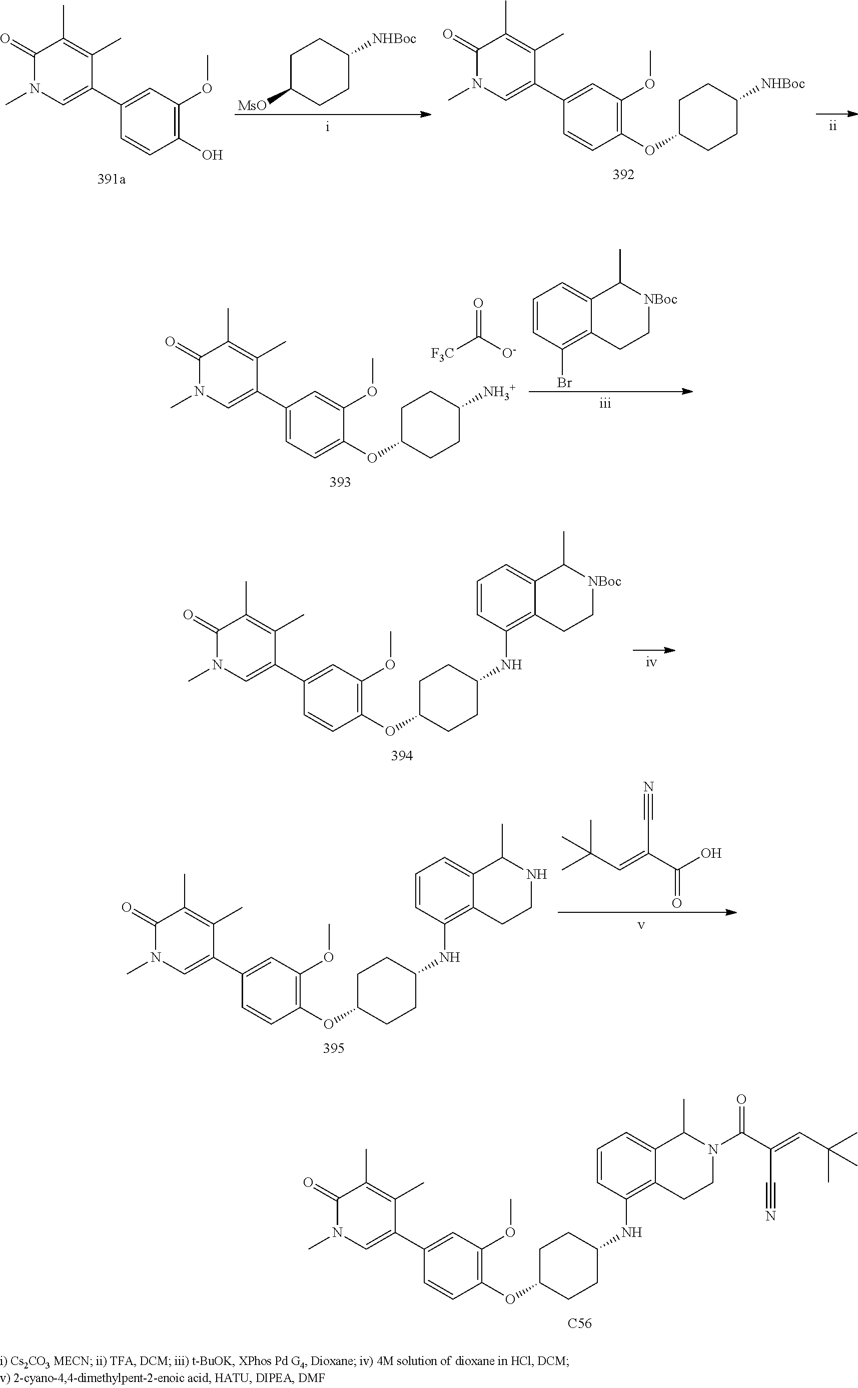

391a: Prepared via 1-step cross-coupling reaction of 5-bromo-1,3,4-trimethylpyridin-2-one (as prepared in WO2021/178920) with 4-hydroxy-3-methoxyphenylboronic acid pinacol ester (commercial) using XPhosPdG3, K3PO4, THF:H2O, 80° C.

392: By general procedure 26 using (1r,4r)-4-((tert-butoxycarbonyl)amino)cyclohexyl methanesulfonate (as described in Tetrahedron Letters, 2010, vol. 51, #51, p. 6741-6744) and Cs2CO3 (2.5 eq) in MeCN. Obtained 250 mg, 28% yield. LCMS m/z=457.2 [M+H]⁺.

393: By general procedure 2b using TFA (2.5 eq) in DCM. Obtained 410 mg. LCMS m/z=357.2 [M+H]⁺.

394: By general procedure 21a using tBuOK instead of cesium carbonate. Obtained 90 mg, 39% yield. LCMS m/z=602.4 [M+H]⁺.

395: By general procedure 2. Obtained 70 mg (crude). LCMS m/z=502.4 [M+H]⁺.

Compound C56: By general procedure 6 using 2-cyano-4,4-dimethylpent-2-enoic acid (1.3 eq) in DMF. Obtained 16 mg, 19% yield. LCMS m/z=637.4 [M+H]⁺.

### Compound C57

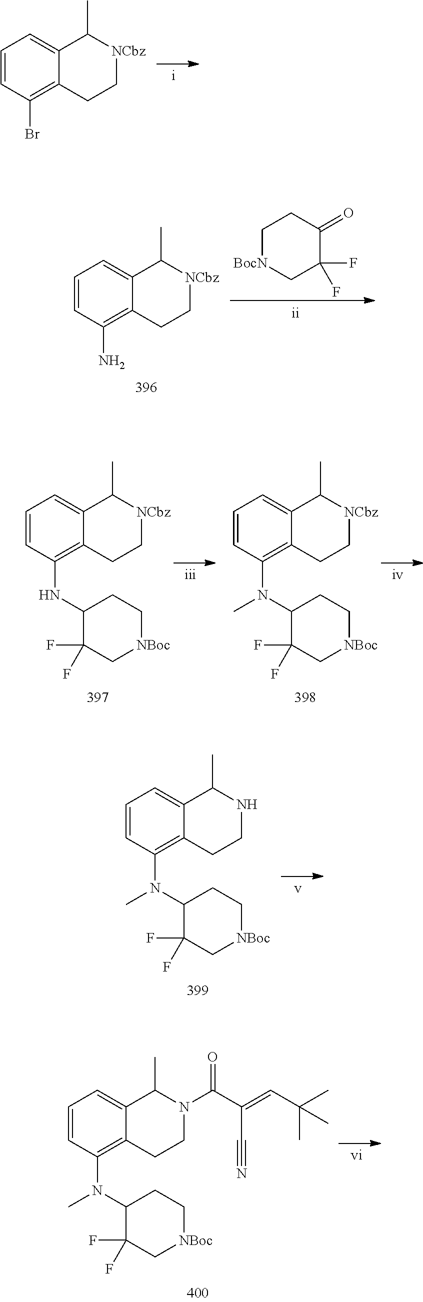

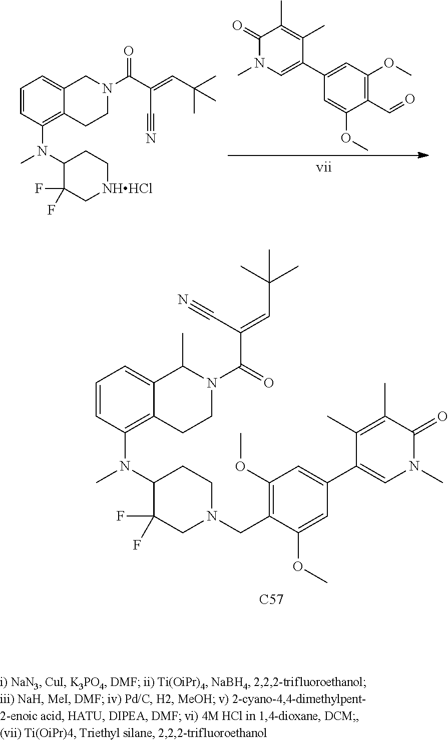

396: To a stirred solution of benzyl 5-bromo-1-methyl-3,4-dihydroisoquinoline-2(1H)-carboxylate (500 mg, 1.38 mmol, 1 eq) and sodium azide (902 mg, 13.8 mmol, 10 eq) in DMF (2.5 mL) was added potassium phosphate tribasic (726 mg, 4.12 mmol, 3 eq), copper(I) iodide (52.8 mg, 0.28 mmol, 0.2 eq) and (1R,2R)-cyclohexane-1,2-diamine (63.4 mg, 0.54 mmol, 0.4 eq). The mixture was stirred overnight at 120° C., then cooled to RT. The reaction mixture was diluted with ethyl acetate and was washed with water. The organic layer was dried over anhydrous sodium sulphate, filtered and concentrated in vacuo. The resulting residue was purified by reverse phase chromatography (gradient=0-55% MeCN in water). The appropriate fractions were concentrated in vacuo to yield benzyl 5-amino-1-methyl-3,4-dihydroisoquinoline-2(1H)-carboxylate (237 mg, 792 mmol, 57% yield). LCMS m/z=297.2 [M+H]⁺.

397: To a stirred solution of benzyl 5-amino-1-methyl-3,4-dihydroisoquinoline-2(1H)-carboxylate (237 mg, 0.80 mmol, 1 eq) and tert-butyl 3,3-difluoro-4-oxopiperidine-1-carboxylate (376 ng, 1.59 mmol, 2 eq) in 2,2,2-trifluoro ethanol (2 mL) was added titanium (IV) isopropoxide (227 mg, 8.00 mmol, 10 eq). The reaction mixture was stirred at 70° C. for 16 h before being cooled to 0° C. Sodium borohydride (151 mg, 4.00 mmol, 5 eq) was added and the reaction mixture was stirred at RT for 1 h. The reaction mixture was quenched with water ethyl acetate was added. The reaction was filtered through celite, and the organic layer was separated, dried with anhydrous sodium sulphate filtered and concentrated in vacuo. The resulting residue was purified by silica gel column chromatography (0-20% EtOAc in hexane). The appropriate fraction were concentrated in vacuo to afford benzyl 5-((1-(tert-butoxycarbonyl)-3,3-difluoropiperidin-4-yl)amino)-1-methyl-3,4-dihydroisoquinoline-2(1H)-carboxylate (280 mg, 0.527 mmol, 65.9% yield). LCMS m/z=416.2 [M-Boc]⁺.

398: To a stirred solution of benzyl 5-((1-(tert-butoxycarbonyl)-3,3-difluoropiperidin-4-yl)amino)-1-methyl-3,4-dihydroisoquinoline-2(1H)-carboxylate (280 mg, 0.543 mmol, 1 eq) in DMF (2 mL) was added sodium hydride (95 mg, 2.172 mmol, 4 eq) at 0° C. The reaction mixture was stirred for 5 min, then MeI (385 mg, 2.72 mmol, 5 eq) was added and the mixture was stirred for 4 h at RT. The reaction mixture was quenched with ice-water and extracted with EtOAc. The combined organic layer was dried over anhydrous sodium sulphate, filtered and concentrated in vacuo. The resulting residue was purified by silica gel column chromatography (gradient=0-20% EtOAc in hexane). The appropriate fractions were concentrated in vacuo to afford benzyl 5-((1-(tert-butoxycarbonyl)-3,3-difluoropiperidin-4-yl)(methyl)amino)-1-methyl-3,4-dihydroisoquinoline-2(1H)-carboxylate (220 mg, 76% yield). LCMS m/z=530.4 [M+H]⁺. 399: By general procedure 27 using Pd/C (2 eq) in MeOH. Obtained 220 mg, 79% yield. LCMS m/z=339.3 [M−56]⁺.

400: By general procedure 6. Obtained 110 mg, 55% yield. LCMS m/z=531.4 [M+H]⁺.

401: By general procedure 2b. Obtained 100 mg. LCMS m/z=431.5 [M+H]⁺.

Compound C57: To a stirred solution of (E/Z)-4-((2-(2-cyano-4,4-dimethylpent-2-enoyl)-1-methyl-1,2,3,4-tetrahydroisoquinolin-5-yl)(methyl)amino)-3,3-difluoropiperidin-1-ium 2,2,2-trifluoroacetate (100 mg, 0.15 mmol, 1 eq) and 2,6-dimethoxy-4-(1,4,5-trimethyl-8-oxo-1,6-dihydropyridin-3-yl)benzaldehyde (0.83 mg, 0.28 mmol, 1.5 eq) in 2,2,2-trifluoro ethanol (1 mL) was added titanium(IV) isopropoxide (0.22 mL, 0.73 mmol, 4 eq). The reaction mixture was stirred at 70° C. for 2 h before being cooled to 0° C. Triethylsilane (0.056 mL, 0.37 mmol, 2 eq) was added and the reaction mixture was stirred at 60° C. for 1 h. The reaction mixture was partitioned between water and EtOAc, and the reaction mixture was added filtered through celite. The organic layer was separated, dried with anhydrous sodium sulphate, filtered and concentrated in vacuo to yield (E/Z)-2-(5-((1-(2,6-dimethoxy-4-(1,4,5-trimethyl-6-oxo-1,6-dihydropyridin-3-yl)benzyl)-3,3-difluoropiperidin-4-yl)(methylamino)-1-methyl-1,2,3,4-tetrahydroisoquinoline-2-carbonyl)-4,4-dimethylpent-2-enenitrile (12 mg, 9% yield). LCMS m/z=716.2 [M+H]⁺.

### Compound C58

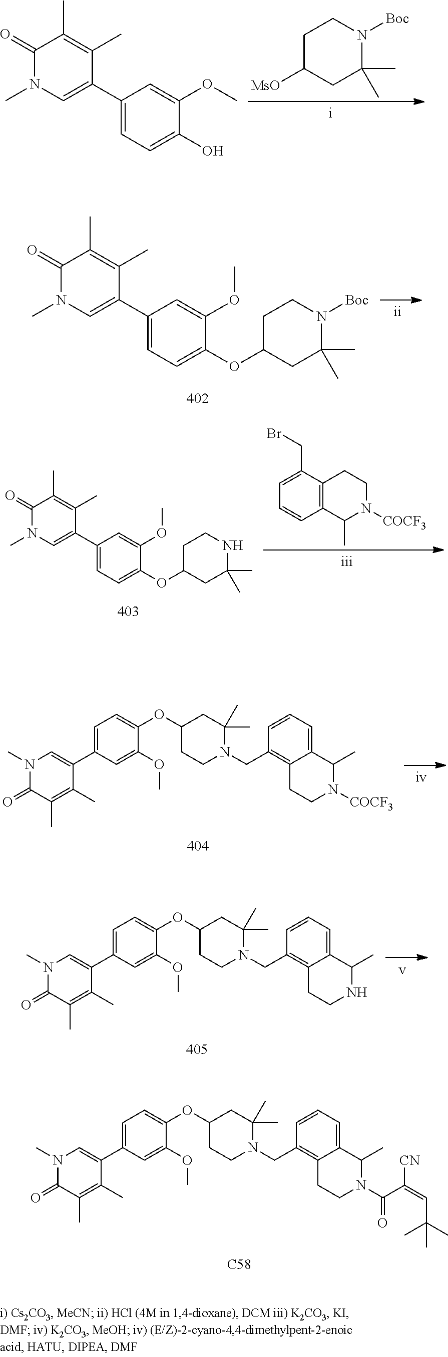

402: By general procedure 26 using starting material made according to WO2019/191112. Obtained 80 mg, 39% yield. LCMS m/z=471.0 [M+H]⁺

403: By general procedure 2 using 4M solution of dioxane in HCl in DCM. Obtained 70 mg, crude. LCMS m/z=371.0 [M+H]⁺

404: To a stirred solution of 1-(5-(bromomethyl)-1-methyl-3,4-dihydroisoquinolin-2(1H)-yl)-2,2,2-trifluoroethan-1-one (100 mg, 0.297 mmol, 1 eq) in DMF (3 ml) was added K₂CO₃ (90 mg, 0.654 mmol, 2.2 eq), potassium iodide (9.88 mg, 0.059 mmol, 0.2 eq) and 5-(4-((2,2-dimethylpiperidin-4-yl)oxy)-3-methoxyphenyl)-1,3,4-trimethylpyridin-2(1H)-one (254 mg, 0.625 mmol, 2.1 eq) at RT. The reaction mixture was heated to 110° C. for 16 h before being quenched with ice cold water and extracted with EtOAc. The organic layer was concentrated in vacuo and the resulting residue was purified by silica gel column chromatography (gradient=0-10% EtOAc in hexane). The appropriate fractions were concentrated in vacuo to afford 5-(4-((2,2-dimethyl-1-((1-methyl-2-(2,2,2-trifluoroacetyl)-1,2,3,4-tetrahydroisoquinolin-5-yl)methyl)piperidin-4-yl)oxy)-3-methoxyphenyl)-1,3,4-trimethylpyridin-2(1H)-one (51 mg, 0.077 mmol, 25.8% yield). LCMS m/z=626.3 [M+H]⁺.

405: By general procedure 20. Obtained 42 mg, crude. LCMS m/z=530.2 [M+H]⁺

Compound C58: By general procedure 6. Obtained 7 mg, 14% yield. LCMS m/z=665.4 [M+H]⁺.

### Compound C59 and Compound C60Compound C59: Single Enantiomer of (E/Z)-2-(5-((1-(2,6-dimethoxy-4-(1,4,5-trimethyl-6-oxo-1,6-dihydropyridin-3-yl)benzyl)piperidin-4-yl)methyl)-1-methyl-1,2,3,4-tetrahydroisoquinoline-2-carbonyl)-4,4-dimethylpent-2-enenitrile

### Compound C60: Single Enantiomer of (E/Z)-2-(5-((1-(2,6dimethoxy-4-(1,4,5-trimethyl-6-oxo-1,6-dihydropyridin-3-yl)benzyl)piperidin-yl)methyl)-1-methyl-1,2,3,4-tetrahydroisoquinoline-2-carbonyl)-5-fluoro-4,4-dimethylpent-2-enenitrile

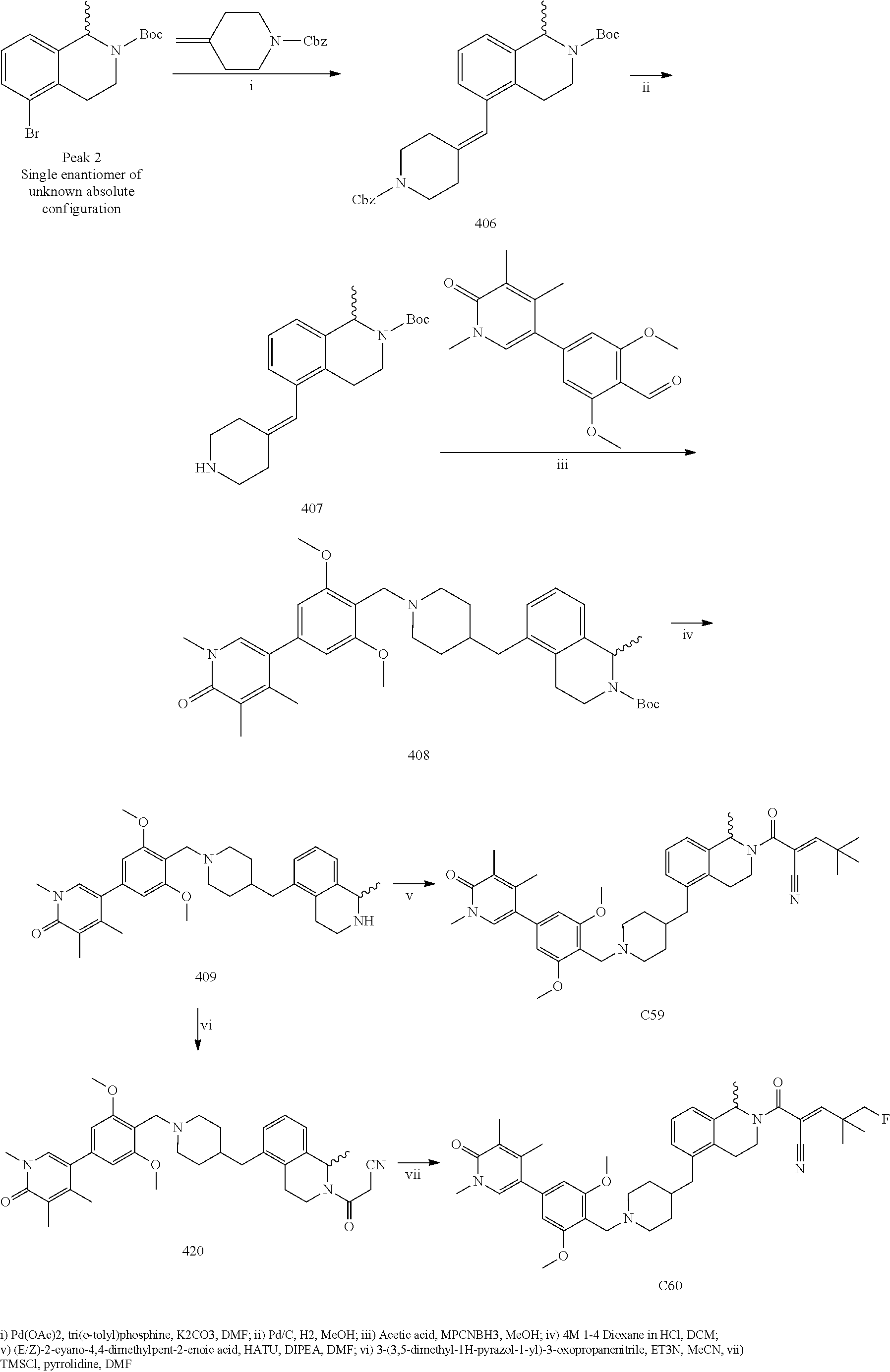

406: Prepared general procedure 31. Obtained 550 mg, 53% yield. LCMS: 377.4 [M-Boc]⁺.

407: By general procedure 27. Obtained 450 mg. LCMS m/z=345.4 [M+H]⁺.

408: By general procedure 1 using MPCNB₃ (w/w) in MeOH. Obtained 800 mg, 83% yield. LCMS m/z=630-3 [M+H]⁺

409: By general procedure 2 using 4M HCl in 1,4-dioxane in DCM. Obtained 300 mg. LCMS m/z=530.3 [M+H]⁺

Compound C59: By general procedure 6. Obtained 18 mg, 11.8% yield. LCMS m/z=665.3 [M+H]⁺

420: By general procedure 3. Obtained 120 mg, 27.9% yield. LCMS m/z=597 [M+H]⁺

Compound C60: By general procedure 4a. Obtained 17 mg, 13.8% yield. LCMS m/z=683.3 [M+H]⁺

### Compound C61

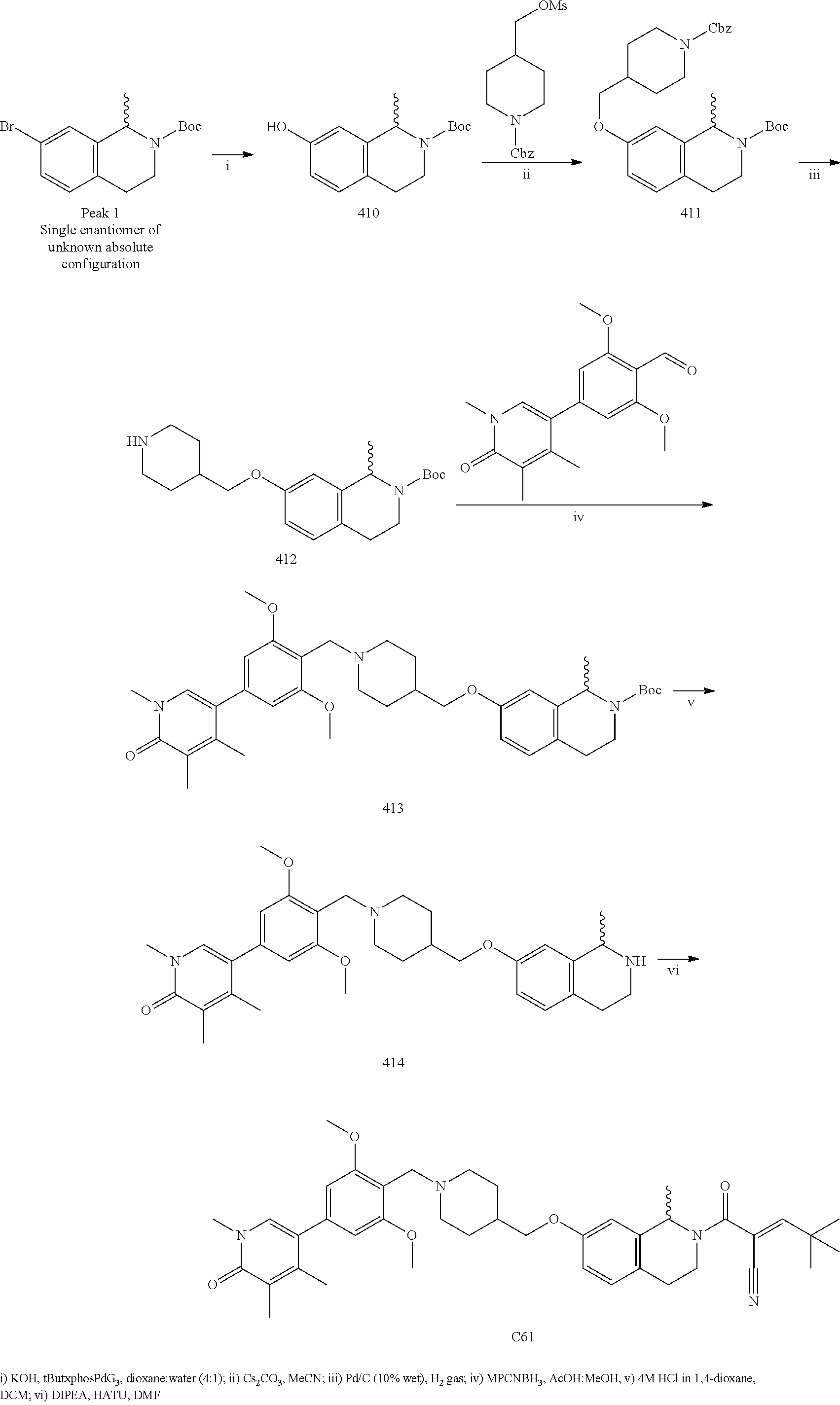

410: By general procedure 24 using KOH (6 eq) in 1,4-dioxane:water. Obtained 1.0 g, 60.7% yield. LCMS m/z=164.2 [M-Boc+H]⁺

411: By general procedure 26 using starting material prepared according to WO2016/40515. Obtained 600 mg, 63.2% yield. LCMS m/z=395.2 [M-Boc+H]⁺

412: By general procedure 27. Obtained 430 mg, 97% yield. LCMS m/z=361.4 [M+H]⁺.

413: By general procedure 1 using MPCNBH₃ (w/w) in MeOH. Obtained 530 mg, 44.6% yield. LCMS m/z=646.3 [M+H]⁺.

414: By general procedure 2 using HCl (4M solution in 1,4-dioxane) in DCM. Obtained 330 mg. LCMS m/z=546.3 [M+H]⁺.

Compound C61: By general procedure 6. Obtained 40 mg, 18.5% yield. LCMS m/z=681.3 [M+H]⁺.

### Compound C62

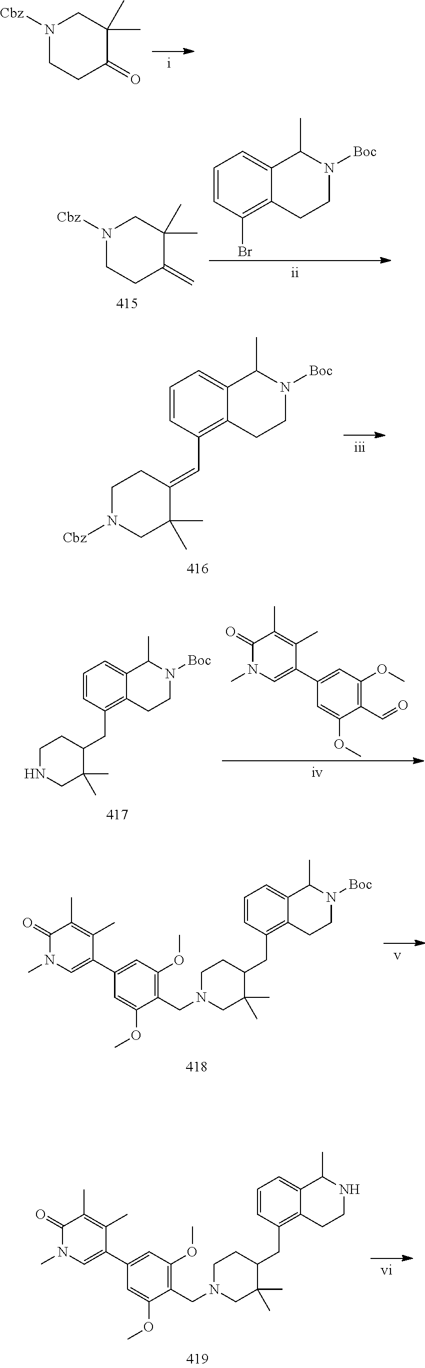

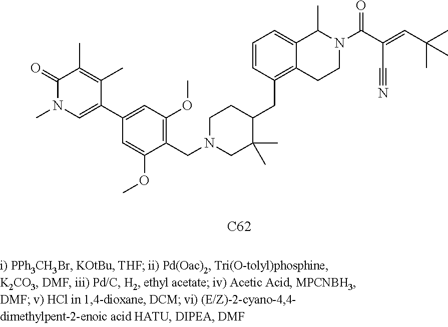 [SMILES: COc1cc(-c2cn(C)c(=O)c(C)c2C)cc(OC)c1CN1CCC(Cc2cccc3c2CCN(C(=O)/C(C#N)=C/C(C)(C)C)C3C)C(C)(C)C1 ; "C62"]

415: By general procedure 30 using starting material prepared from 02018/136887 and methyltriphenylphosphonium bromide (1 eq) in THF. Obtained 990 mg, 99% yield.

416: Prepared general procedure 31. Obtained 400 mg, 51.7% yield. LCMS m/z=405.2 [M+H]⁺.

417: By general procedure 27 in ethyl acetate. Obtained 300 mg, 81% yield. LCMS m/z=373.4 [M+H]⁺.

418: By general procedure 14 using MPCNBH3 in DCE:MeOH. Obtained 350 mg, 57.7% yield. LCMS m/z=658.3 [M+H]⁺

419: By general procedure 2 using 4M HCl in dioxane in DCM. Obtained 180 mg, crude. LCMS m/z=558.3 [M+H]⁺

Compound C62: By general procedure 6. Obtained 78 mg, 38.6% yield. LCMS m/z=693.4 [M+H]⁺.

### Compound C63 and Compound C64

### Enantiomer 1 and Enantiomer 2 of (E/Z)-2-(7-((3-(2,6-dimethoxy-4-(1,4,5-trimethyl-6-oxo-1,6-dihydropyridin-3-yl)benzyl)-3,6-diazabicyclo[3.1.1]heptan-6-yl)methyl)-1-methyl-1,2,3,4-tetrahydroisoquinoline-2-carbonyl)-4,4-dimethylpent-2-enenitrile

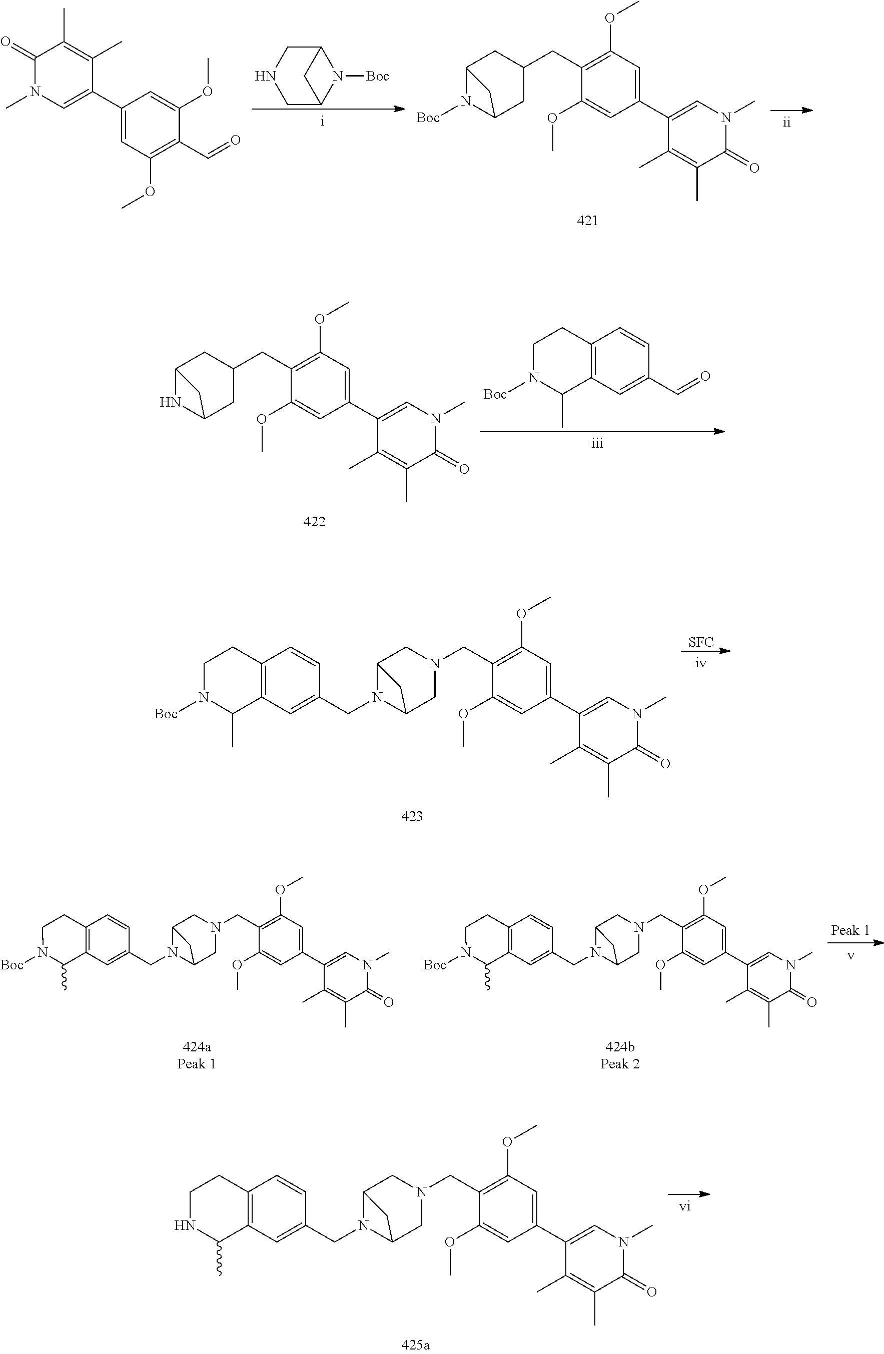

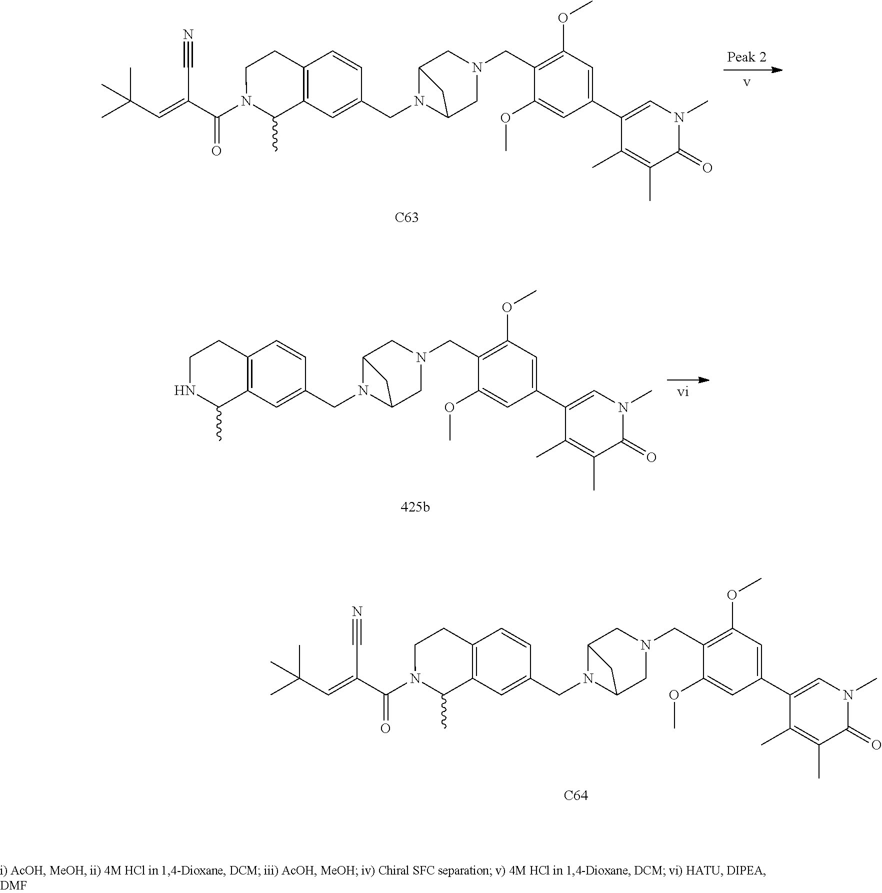

421: By general procedure 1 using MP-CNBH₃ (w/w) in MeOH. Obtained 800 mg, 96% yield. LCMS m/z=484.4 [M+H]⁺.

422: By general procedure 2 using HCl (4M solution in 1,4-dioxane), in DCM. Obtained 630 mg. LCMS m/z=384.4 [M+H]⁺.

423: By general procedure 1 using MP-CNBH₃ (w/w) in MeOH. Obtained 410 mg, 53.3% yield. LCMS m/z=643.4 [M+H]⁺.

Racemic material (400 mg) was then purified by SFC:

| | |
|---|---|
| Instrument | S/DC/ARD/22-030 |
| Column | I Cellulose Z-(250*30)mm, 5 μm |
| Mobile Phase | CO₂: 0.5% isopropylamine in MeOH:MeCN [60:40] |
| Flow rate | 120 mL/min |
| Back pressure | 120 bar |
| Wavelength | 254 nm |
| Cycle time | 6 min |

424a: Isolated Peak-1: 85 mg, 20.8% yield. LCMS m/z=643.4 [M+H]⁺

424b: Isolated Peak-2: 86 mg, 20.6% yield. LCMS m/z=643.4 [M+H]⁺

425a: By general procedure 2. Obtained 70 mg. LCMS m/z=543.6 [M+H]⁺.

Compound C63: By general procedure 6. Obtained 38 mg, 42.2% yield. LCMS m/z=678.4 [M+H]⁺.

425b: By general procedure 2. Obtained 70 mg. LCMS m/z=543.6 [M+H]⁺.

Compound C64: By general procedure 6. Obtained 36 mg, 39.7% yield. LCMS m/z=678.4 [M+H]⁺.

### Single Enantiomer of (E/Z)-2-(7-((1-(2,6-dimethoxy-4-(1,4,5-trimethyl-6-oxo-1,6-dihydropyridin-3-yl)benzyl)piperidin-4-yl)methyl)amino)-1-methyl-1,2,3,4-tetrahydroisoquinoline-2-carbonyl)-4,4-dimethylpent-2-enenitrile (C65)

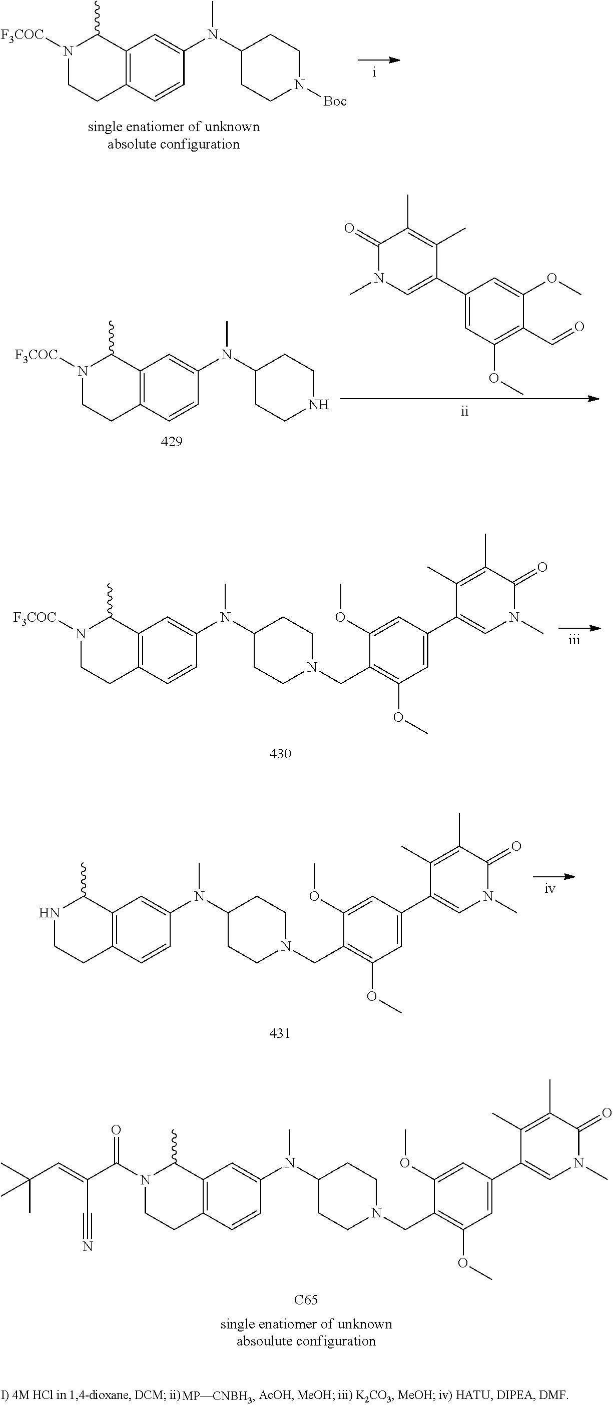

429: By general procedure 2 using 4M HCl in 1,4-dioxane in DCM. Obtained 350 mg, 55% yield. LCMS m/z=358.2 [M+H]⁺.

430: By general procedure 14 using MP-CNBH₃ (w/w) in DCE. Obtained 320 ng, 80% yield. LCMS m/z=641.2 [M+H]⁺.

431: By general procedure 20. Obtained 230 mg. LCMS m/z=545.3 [M+H]⁺.

Compound C66: By general procedure 6. Obtained 11 mg, 5.8% yield. LCMS m/z=680.4 [M+H]⁺.

### Compound C66 and Compound C67

### Enantiomer 1 and Enantiomer 2 of (E/Z)-2-(7-(6-(2,6-dimethoxy-4-(1,4,5-trimethyl-6-oxo-1,6-dihydropyridin-3-yl)benzyl)-2,8-diazaspiro[4.5]decan-2 yl)-1-methyl-1,2,3,4-tetrahydroisoquinoline-2-carbonyl)-4,4-dimethylpent-2-enenitrile

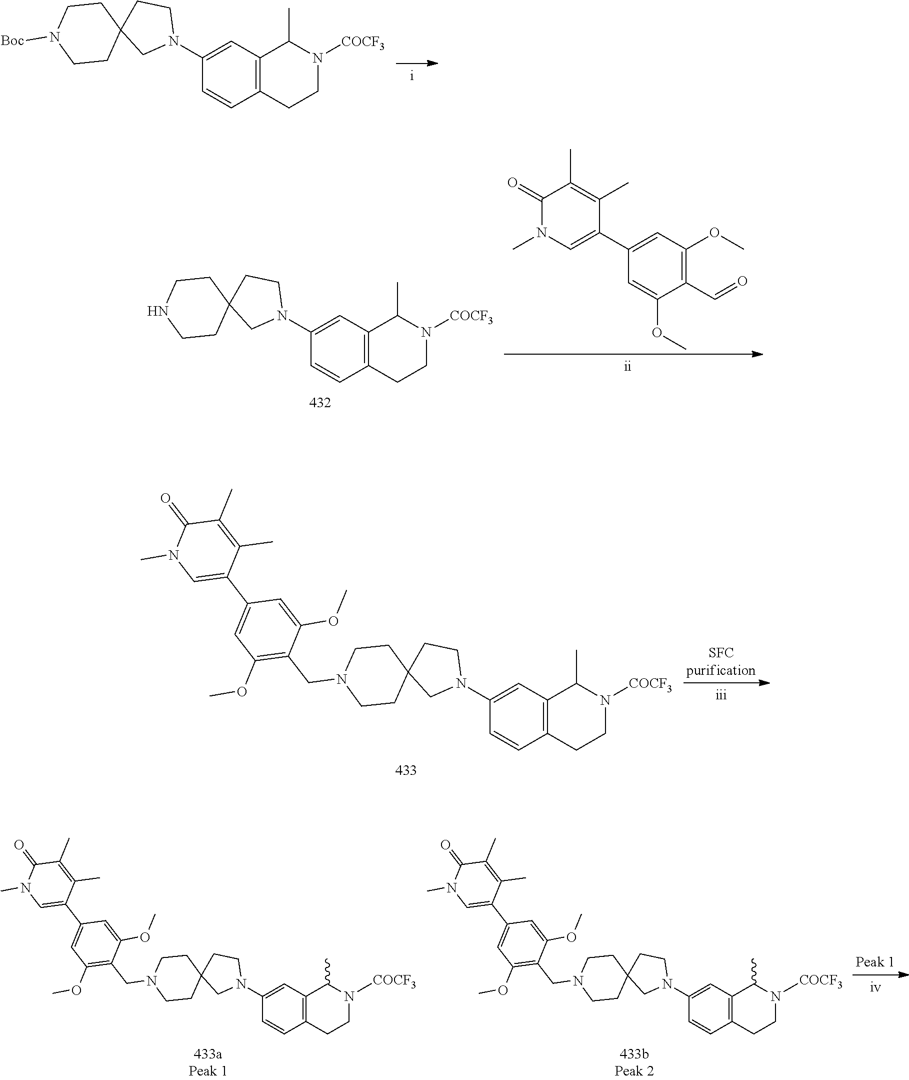

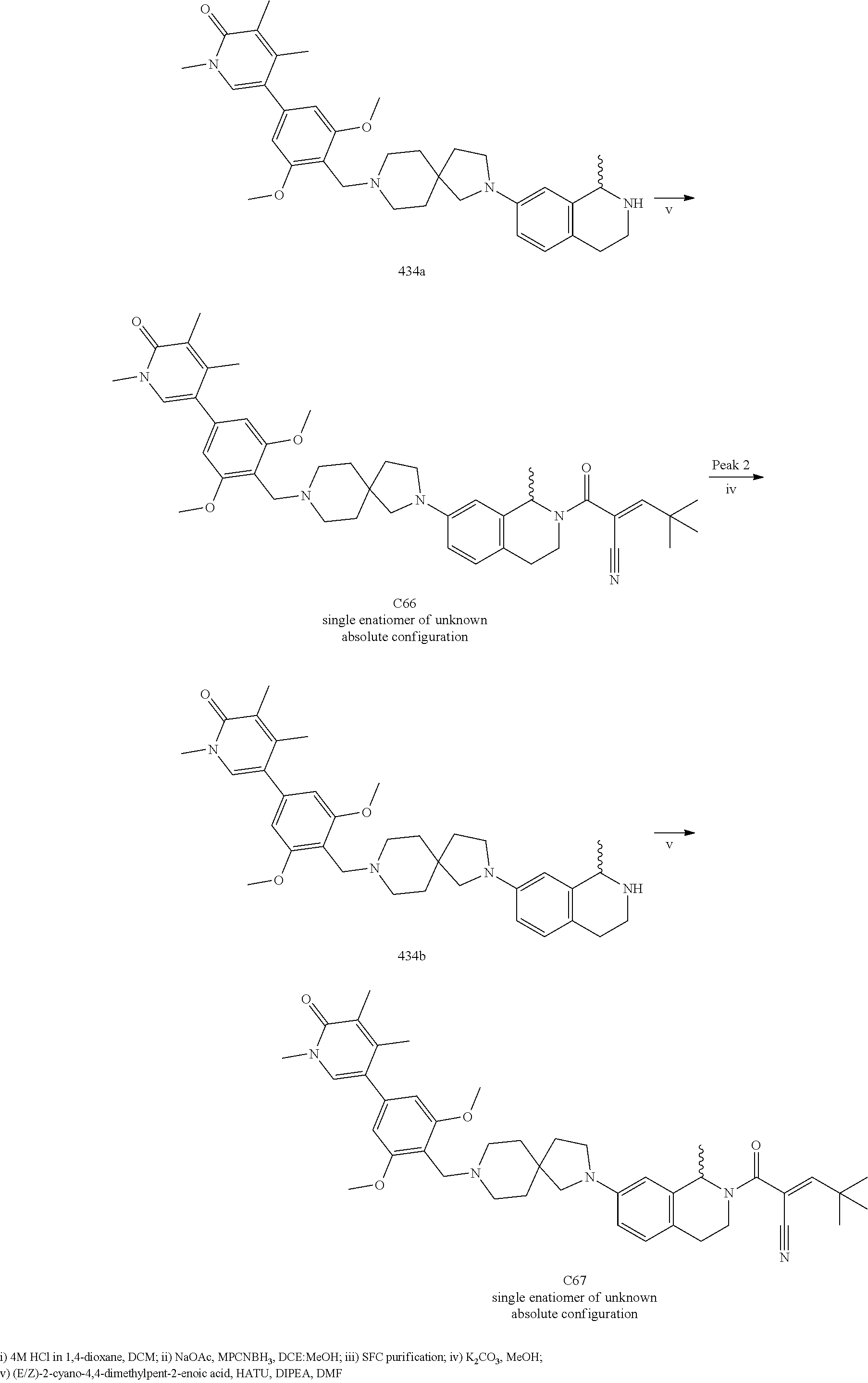

432: By general procedure 2 using 4M HCl in 1,4-dioxane in DCM. Obtained 300 mg, crude. LCMS m/z=382.2 [M+H]⁺

433: By general procedure 14 using MPCNBH3 (w/w) in DCE:MeOH. Obtained 300 mg, 57.2% yield. LCMS m/z=687.2 [M+H]⁺

Racemic 5-(3,5-dimethoxy-4-((2-(1-methyl-2-(2,2,2-trifluoroacetyl)-1,2,3,4-tetrahydroisoquinolin-7-yl)-2,8-diazaspiro[4.5]decan-8-yl)methyl)phenyl)-1,3,4-trimethylpyridin-2(1H)-one (300 mg) was purified by SFC:

| Column | WHELK RR-(250*30)mm, 5 μm | |
|---|---|---|
| Mobile Phase | CO₂: 0.5% isopropylamine in | |
| | MeOH:MeCN [50:50] | |
| Flow rate | 50 | mL/min |
| Back pressure | 100 | bar |
| Wavelength | 254 | nm |
| Cycle time | 9 | min |

433a: Isolated Peak-1: 100 mg, LCMS m/z=667.2 [M+H]⁺

433b: Isolated Peak-2: 120 mg, LCMS m/z=667.3 [M+H]⁺

434a: By general procedure 2c using peak 1 isolated above. Obtained 55 mg, 61.2% yield. LCMS m/z=571.2 [M+H]⁺

Compound C66: By general procedure 6 using peak 1 isolated above. Obtained 13 mg, 20.6% yield. LCMS m/z=706.2 [M+H]⁺

434b: By general procedure 2c using peak 2 isolated above. Obtained 70 mg, crude. LCMS m/z=571.2 [M+H]⁺

Compound C67: By general procedure 6 using peak 2 isolated above. Obtained 11 mg, 13.2% yield. LCMS m/z=706.4 [M+H]⁺

### (E/Z)-2-cyano-5-fluoro-4,4-dimethylpent-2-enoic Acid

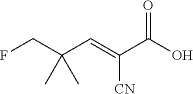

To a stirred solution of 2-cyanoacetic acid (1 g, 11.76 mmol, 1 eq) in methanol (5 mL) was added piperidine (3 g, 35.3 mmol, 3 eq) and the reaction mixture was stirred for 5 min at 25° C. 3-fluoro-2,2-dimethylpropanal (2.44 g, 23.51 mmol, 2 eq) was added and the reaction mixture was stirred at 45° C. for 16 h. The reaction mixture was diluted with water (20 mL) and extracted with EtOAc (20 mL×2). The aqueous layer pH was adjusted ˜3 using HCl (1.5 M) and the mixture was extracted with EtOAc (10 mL×2). The combined organic layers were dried over anhydrous sodium sulphate, filtered and concentrated in vacuo. The resulting residue was purified by reverse phase column chromatography (C18, flow rate=20 mL/min, gradient=30% MeCN in water). The appropriate fractions were concentrated in vacuo to afford (E/Z)-2-cyano-5-fluoro-4,4-dimethylpent-2-enoic acid (230 mg, 1.344 mmol, 11.4% yield). LCMS m/z=170.2 [M−H]⁻

### Compound C68 and Compound C69

### Enantiomer 1 and Enantiomer 2 of (E/Z)-2-(7-(4-(2,6-dimethoxy-4-(1,4,5-trimethyl-6-oxo-1,6-dihydropyridin-3-yl)benzyl)-1,4-diazepan-1-yl)-1-methyl-1,2,3,4-tetrahydroisoquinoline-2-carbonyl)-5-fluoro-4,4-dimethylpent-2-enenitrile

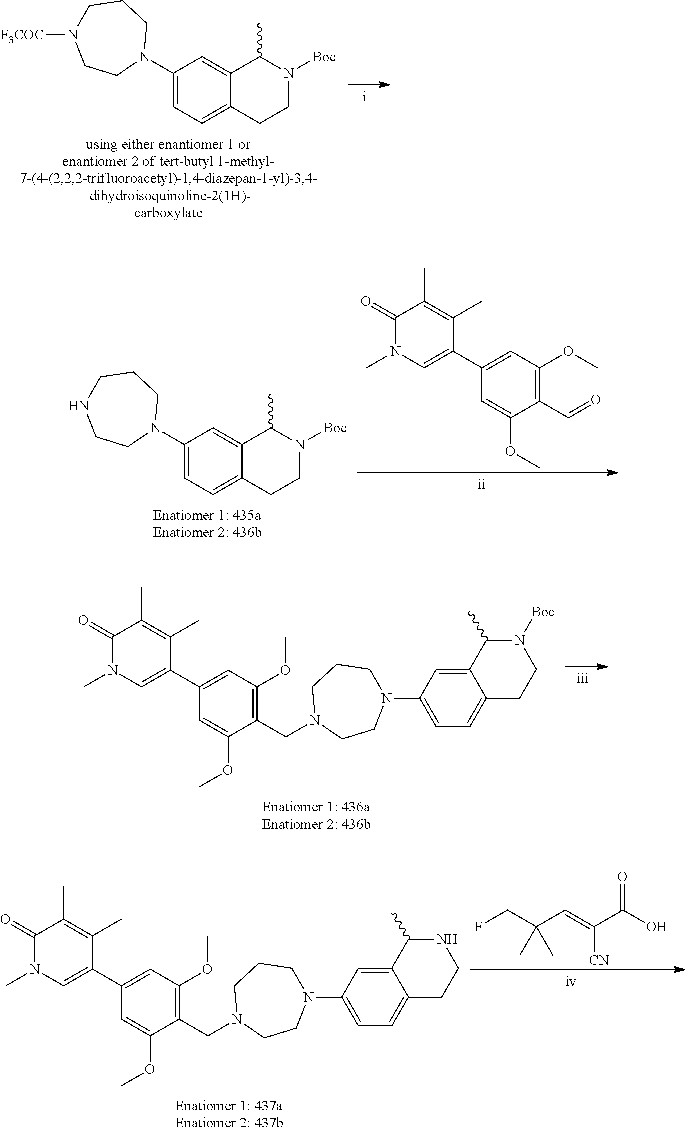

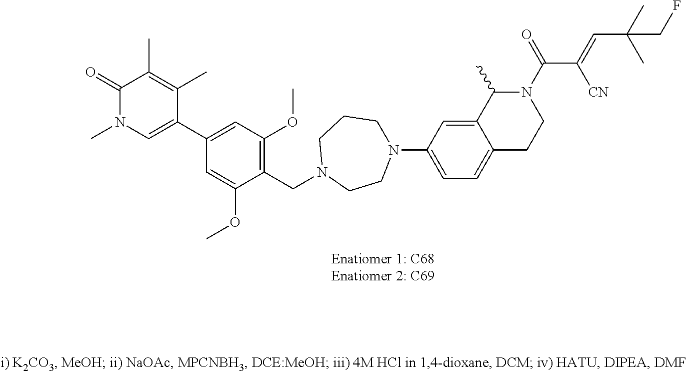 [SMILES: COc1cc(-c2cn(C)c(=O)c(C)c2C)cc(OC)c1CN1CCCN(c2ccc3c(c2)C(C)N(C(=O)/C(C#N)=C/C(C)(C)CF)CC3)CC1]

435a: By general procedure 20 using starting material made from peak 1 of tert-butyl 7-bromo-1-methyl-3,4-dihydroisoquinoline-2(1H)-carboxylate. Obtained 300 mg, 72.9% yield. LCMS m/z=346.2 [M+H]⁺

436a: By general procedure 14 using MPCNBH₃ (w/w) in DCE: MeOH. Obtained 320 mg, 56.1% yield. LCMS m/z=631.4 [M+H]⁺

437a: By general procedure 2 using 4M HCl in 1,4-dioxane in DCM. Obtained 80 mg. LCMS m/z=531.3 [M+H]⁺

Compound C68: By general procedure 6 using (E/Z)-2-cyano-5-fluoro-4,4-dimethylpent-2-enoic acid (2 eq) in DMF. Obtained 15 mg, 14.3% yield. LCMS m/z=684.3 [M+H]⁺

435b: By general procedure 20 using starting material made from peak 2 of tert-butyl 7-bromo-1-methyl-3,4-dihydroisoquinoline-2(1H)-carboxylate. Obtained 320 mg, 71.2% yield. LCMS m/z=346.2 [M+H]⁺

436b: By general procedure 14 using MPCNBH₃ (w/w) in DCE: MeOH. Obtained 300 mg, 51% yield. LCMS m/z=631.4 [M+H]⁺

437b: By general procedure 2 using 4M HCl in 1,4-dioxane in DCM. Obtained 85 mg. LCMS m/z=531.4 [M+H]⁺

Compound C69: By general procedure 6 using (E/Z)-2-cyano-5-fluoro-4,4-dimethylpent-2-enoic acid (2 eq) in DMF. Obtained 14 mg, 12.8% yield. LCMS m/z=684.4 [M+H]⁺

### Compound C70 and Compound C71

### Synthesis of Enantiomer 1 and Enantiomer 2 of (E/Z)-2-(7-(((1-(2,6-dimethoxy-4-(1,4,5-trimethyl-6-oxo-1,6-dihydropyridin-3-yl)benzyl)460zetidine-4-yl)(methyl)amino)methyl)-1-methyl-1,2,3,4-tetrahydroisoquinoline-2-carbonyl)-4,4-dimethylpent-2-enenitrile

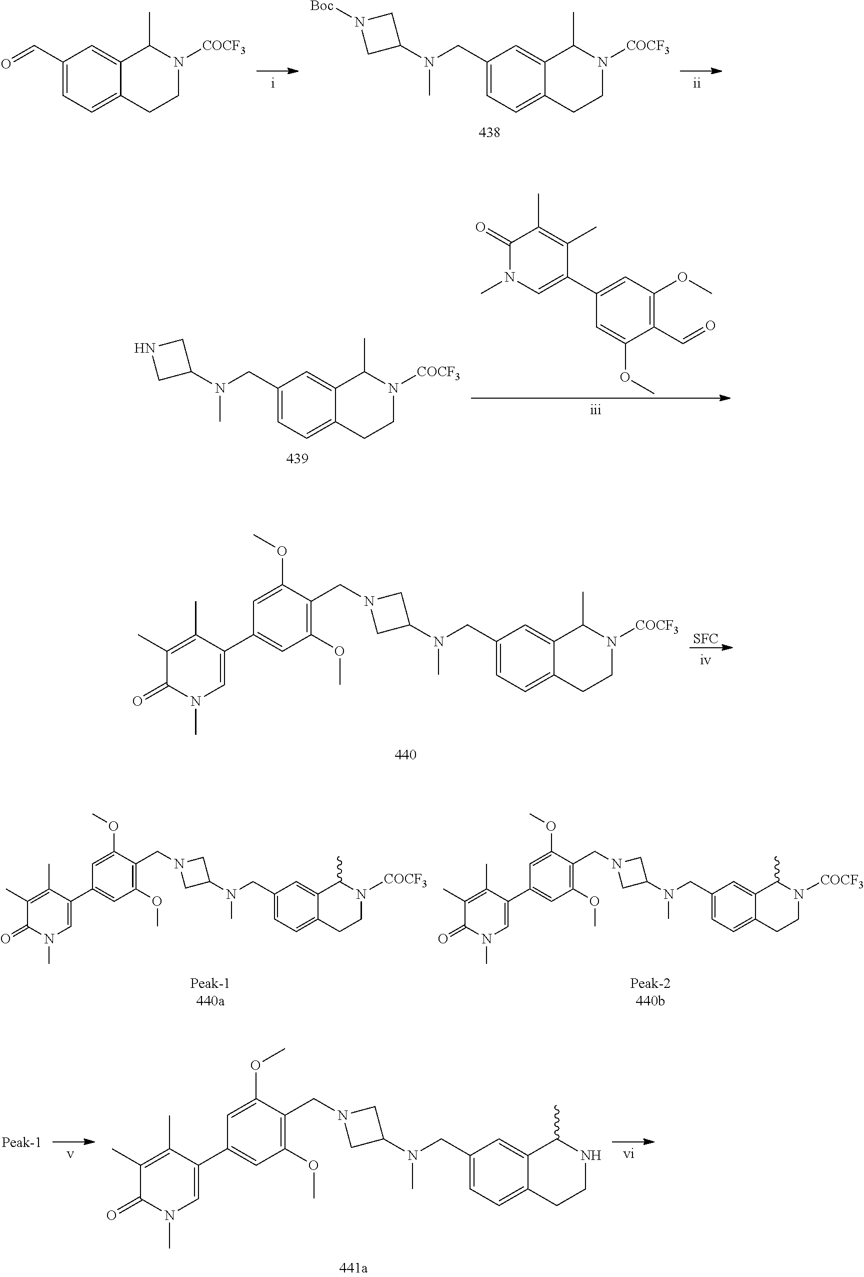

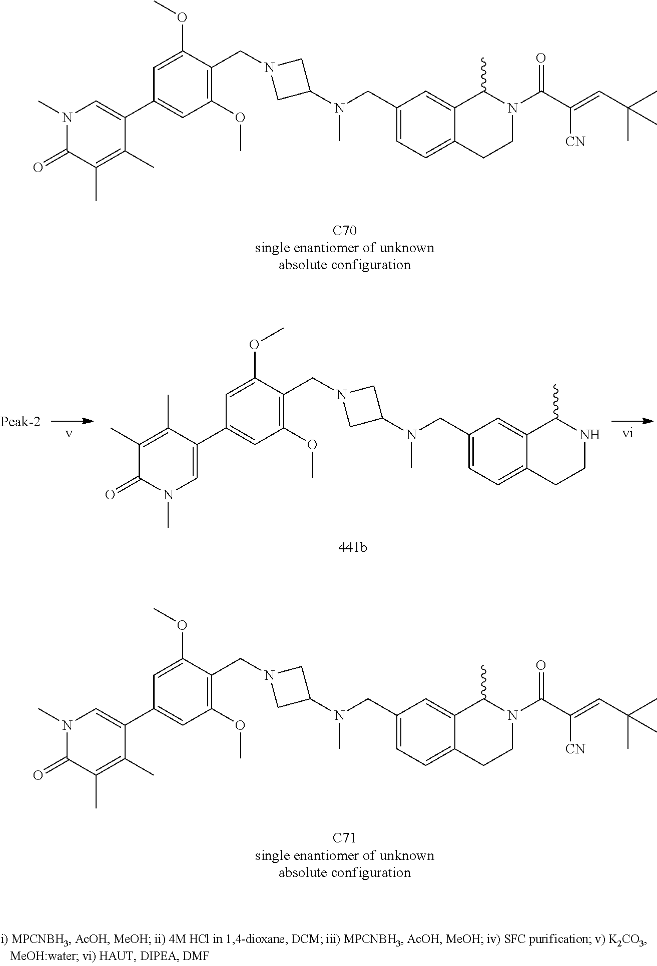

438: By general procedure 1 using MPCNBH₃ (w/w) in MeOH Obtained 1.32 g, 74.4% yield. LCMS m/z=442.2 [M+H]⁺

439: By general procedure 2 using 4M solution of HCl in 1,4-dioxane in DCM. Obtained 1.62 g, LCMS m/z=342.2 [M+H]⁺

440: By general procedure 1 using MPCNBH₃ (w/w) in MeOH. Obtained 700 mg, 54.4% yield. LCMS m/z=627.3 [M+H]⁺

Racemic material (1.1 g) was then purified by asymmetric SFC:

| Column | I Cellulose Z-(250*30)mm, 5 μm | |
|---|---|---|
| Mobile Phase | CO₂: 0.5% isopropylamine in | |
| | MeOH:MeCN [65:35] | |
| Flow rate | 100 | mL/min |
| Back pressure | 100 | bar |
| Wavelength | 220 | nm |
| Cycle time | 6.5 | min |

440b: Isolated Peak-2: 400 mg. LCMS m/z=627.4 [M+H]⁺

441a: By general procedure 20. Obtained 224 mg, 79% yield. LCMS m/z=531.6 [M+H]⁺.

Compound C70: By general procedure 6. Obtained 50 mg, 12.7% yield. LCMS m/z=666.3 [M+H]⁺

441b: By general procedure 20. Obtained 200 mg, 73% yield. LCMS m/z=531.6 [M+H]⁺

Compound C71: By general procedure 8. Obtained 50 mg, 13.1% yield. LCMS m/z=531 [M+H]⁺

### (E/Z)-2-(5-((7-(2-methoxy-4-(1,4,5-trimethyl-6-oxo-1,6-dihydropyridin-3-yl)phenoxy)-4-azaspiro[2.5]octan-4-yl)methyl)-1-methyl-1,2,3,4-tetrahydroisoquinoline-2-carbonyl)-4,4-dimethylpent-2-enenitrile (C72)

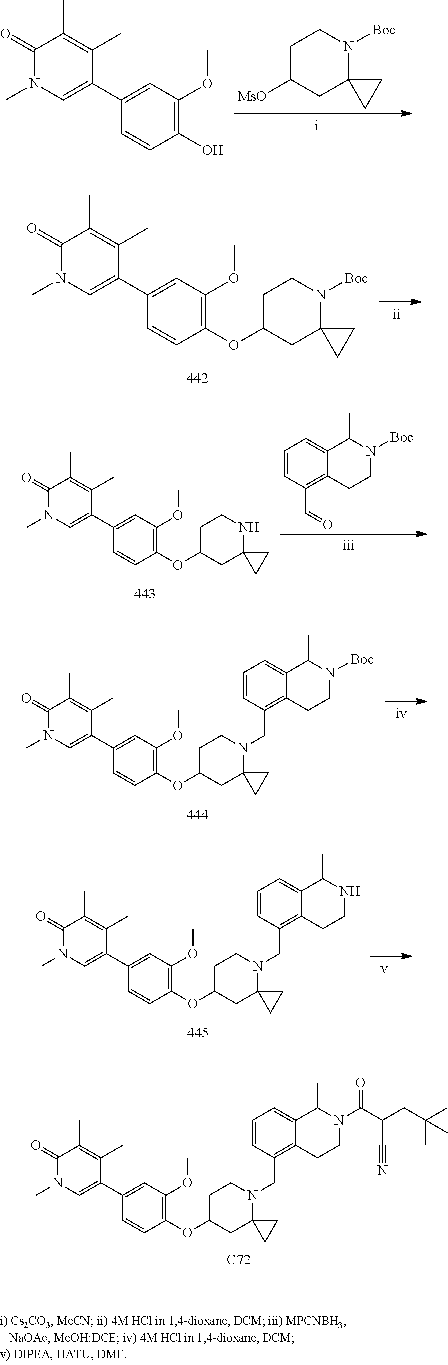

442: By general procedure 26 using starting material prepared as described in WO2022/170122. Obtained 290 mg, 45.8% yield. LCMS m/z=469.2 [M+H]⁺.

443: By general procedure 2 using HCl (4M solution in 1,4-dioxane), in DCM. Obtained 200 mg. 45.8% yield. LCMS m/z=369.4 [M+H]⁺

444: By general procedure 14 using MP-CNBH₃ (1 equiv.) in DCE:MeOH. Obtained 170 mg, 67% yield. LCMS m/z=628.4 [M+H]⁺.

445: By general procedure 2. Obtained 150 mg (crude). LCMS m/z=528.3 [M+H]⁺.

Compound C72: By general procedure 6. Obtained 35 mg, 19.8% yield. LCMS m/z=663.4 [M+H]⁺

### Single Enantiomer of (E/Z)-2-(7-((1-(2,6-dimethoxy-4-(1,4,5-trimethyl-6-oxo-1,6-dihydropyridin-3-yl)benzyl)piperidin-4-yl)oxy)-1-methyl-1,2,3,4-tetrahydroisoquinoline-2-carbonyl)-4,4-dimethylpent-2-enenitrile (C73

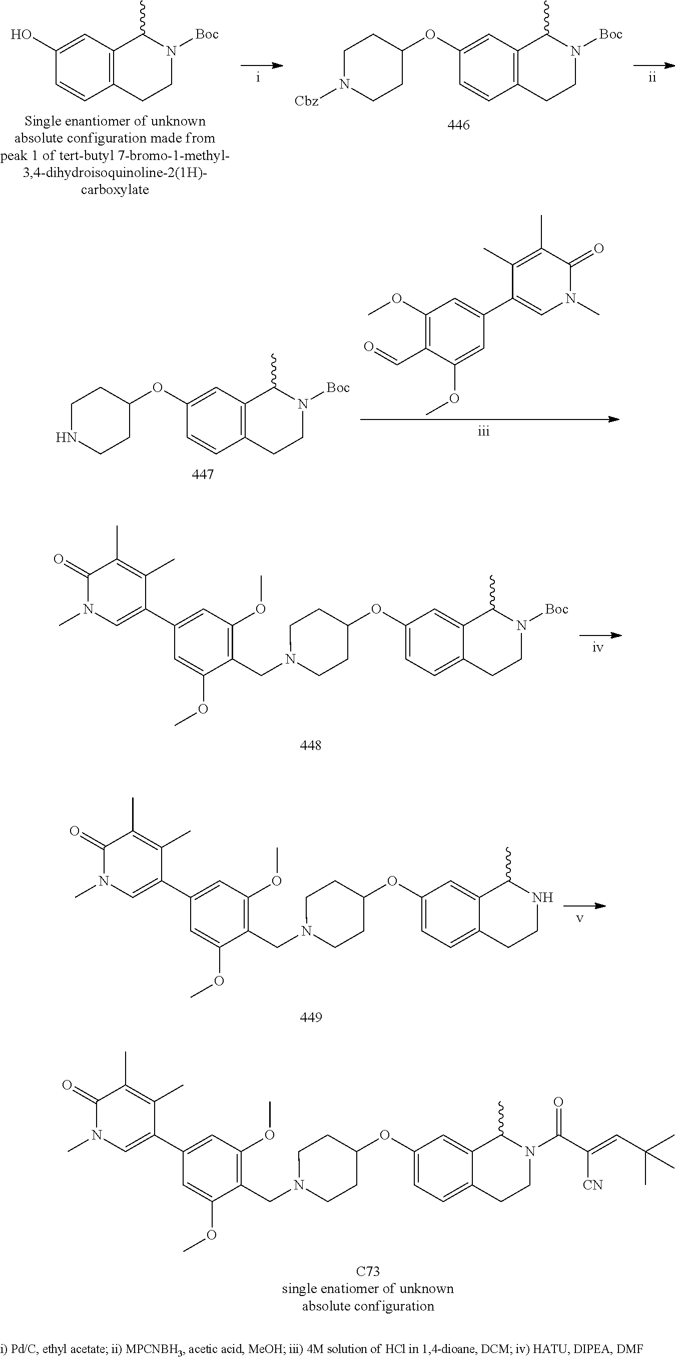

446: By general procedure 26. Obtained 410 mg, 51% yield. LCMS m/z=381.2 [M-Boc]⁺

447: By general procedure 27 using Pd/C (7.7 eq) in EtOAc. Obtained 290 mg. LCMS m/z=347.4 [M+H]⁺

448: By general procedure 1 using MP-CNBH₃ (w/w) in MeOH. Obtained 385 mg, 95% yield. LCMS m/z=632.4 [M+H]⁺.

449: By general procedure 2 using 4M solution of HCl in 1,4-dioxane in DCM. Obtained 325 mg. LCMS m/z=532.2 [M+H]⁺.

Compound C73: By general procedure 6. Obtained 54 mg, 30% yield. LCMS m/z=667.4 [M+H]⁺

### (E/Z)-2-(6-((4-(2-methoxy-4(1,4,5-trimethyl-6-oxo-1,6-dihydropyridin-3-yl)benzyl)-2,2-dimethylpiperidin-1-yl)methyl)-1-methyl-1,2,3,4-tetrahydroisoquinoline-2-carbonyl)-4,4-dimethylpent-2-enenitrile (C74)

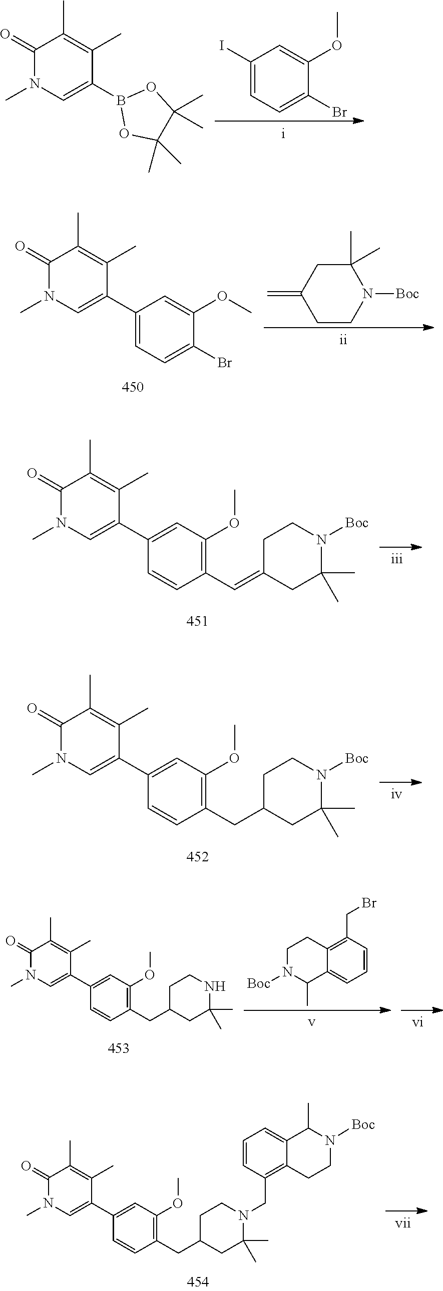

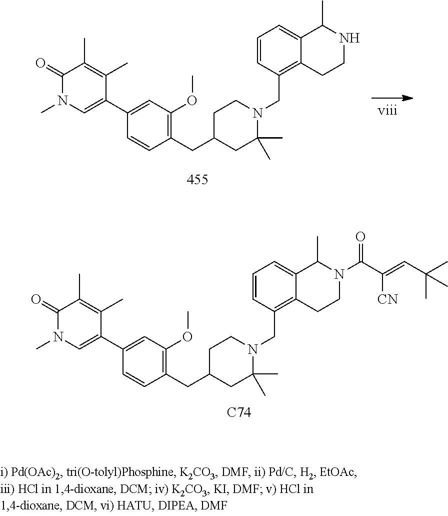 [SMILES: COc1cc(-c2cn(C)c(=O)c(C)c2C)ccc1CC1CCN(Cc2cccc3c2CCNC3C)C(C)(C)C1 ; COc1cc(-c2cn(C)c(=O)c(C)c2C)ccc1CC1CCN(Cc2cccc3c2CCN(C(=O)/C(C#N)=C/C(C)(C)C)C3C)C(C)(C)C1 ; "C74"]

450: To a stirred solution of 1,3,4-trimethyl-5-(4,4,5,5-tetramethyl-1,3,2-dioxaborolan-2-yl) pyridin-2 (1H)-one (1.0 g, 3.83 mmol, 1 eq) and 1-bromo-4-iodo-2-methoxybenzene (1 g, 3.20 mmol, 1 eq) in water (2 mL) added tripotassium phosphate (1.357 g, 6.39 mmol, 2 eq) and THF (8 mL). The reaction flask was purged with N₂ for 10 min, then added Pd(PPh₃)₄ (0.18 g, 0.16 mmol, 0.05 eq) was added and the reaction mixture was stirred for 16 h at 80° C. The reaction mixture was filtered through celite and the celite pad was washed with EtOAc. The combined organic fractions were concentrated in vacuo then diluted with water and extracted with MTBE. The combined organic fractions were concentrated in vacuo to afford 5-(4-bromo-3-methoxyphenyl)-1,3,4-trimethylpyridin-2 (1H)-one (500 mg, 1.552 mmol, 48.6% yield). The crude compound was used without further purification

451: Prepared general procedure 31. Obtained 300 mg, 27.3% yield. LCMS m/z=487.4 [M+H]⁺.

452: By general procedure 27 in EtOAc. Obtained 280 mg, 89% yield. LCMS m/z=489.4 [M+H]⁺.

453: By general procedure 2 using 4M HCl in 1,4-dioxane, in DCM. Obtained 250 mg, 93% yield. LCMS m/z=389.3 [M+H]⁺

454: By general procedure 26. Obtained 389 mg, 44.3% yield. LCMS m/z=828.4 [M+H]⁺.

455: By general procedure 2 using 4M HCl in dioxane in DCM. Obtained 160 mg, 83% yield. LCMS m/z=528.2 [M+H]⁺

Compound C74: By general procedure B. Obtained 34 mg, 28.4% yield. LCMS m/z=663.4 [M+H]⁺.

### (E/Z)-2-(5-((4-(2,6-dimethoxy-4-(1,4,5-trimethyl-6-oxo-1,6-dihydropyridin-3-yl)benzyl)piperazin-1-yl)sulfonyl)-1-methyl-1,2,3,4-tetrahydroisoquinoline-2-carbonyl)-4,4-dimethylpent-2-enenitrile (C75)

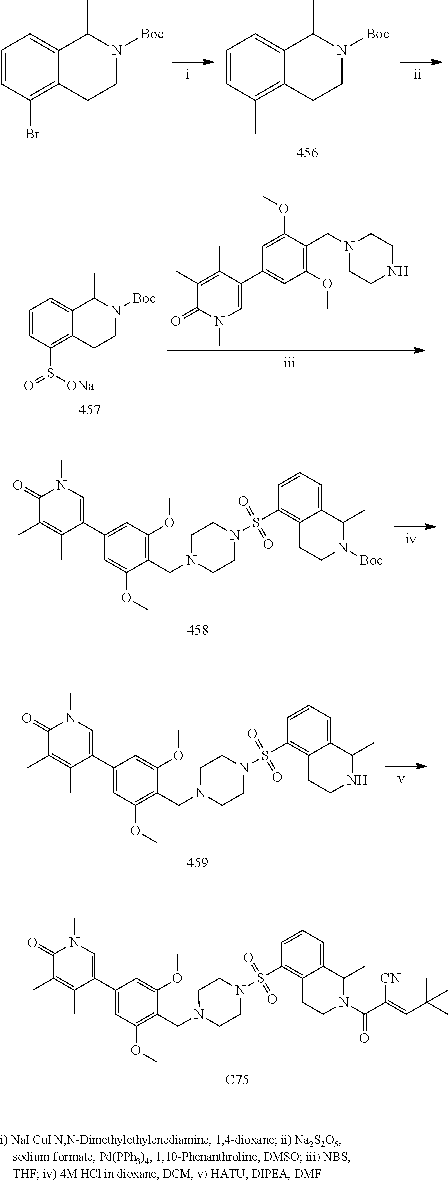

456: To a stirred solution of tert-butyl 5-bromo-1-methyl-3,4-dihydroisoquinoline-2(1H)-carboxylate (1.0 g, 3.07 mmol, 1 eq) in 1,4-dioxane (3 mL), was added sodium iodide (1.83 g, 12.26 mmol, 4 eq), copper(I) iodide (0.12 g, 0.61 mmol, 0.2 eq) and N,N′-Dimethylethylenediamine (0.13 mL, 1.22 mmol, 0.4 eq) at RT. The mixture was stirred at 120° C. for 48 h before being quenched with ice-cold water and extracted with EtOAc. The organic phase was dried over anhydrous Na₂SO₄, filtered and concentrated in vacuo to obtain crude product tert-butyl 5-iodo-1-methyl-3,4-dihydroisoquinoline-2(1H)-carboxylate (880 mg, 2.21 mmol, 72.3% yield). LCMS m/z=274.0 [M-Boc+H]⁺

457: To a stirred solution of tert-butyl 5-iodo-1-methyl-3,4-dihydroisoquinoline-2(1H)-carboxylate (50 mg, 0.134 mmol, 1 eq) in DMSO (1 mL) was added sodium metabisulfite (50.9 mg, 0.26 mmol, 2 eq) and sodium formate (20.34 mg, 0.29 mmol, 2.2 eq). The reaction mixture was purged with nitrogen gas for 10 min before Pd(PPh₃)₄ (7.74 mg, 6.70 μmol, 0.05 eq) and 1,10-phenanthroline (7.24 mg, 0.040 mmol, 0.3 eq) were added and the reaction mixture was degassed and stirred for 10 min before being heated to 70° C. for 2 h. The reaction mixture was concentrated in vacuo and used without further purification. Obtained 40 mg, LCMS m/z=310.3 [M−H]⁺.

458: To a stirred solution of sodium 2-(tert-butoxycarbonyl)-1-methyl-1,2,3,4-tetrahydroisoquinoline-5-sulfinate (475 mg, 1.42 mmol, 1 eq) (solution of DMSO crude) in THF (5 mL) was added 5-(3,5-dimethoxy-4-(piperazin-1-ylmethyl)phenyl)-1,3,4-trimethylpyridin-2(1H)-one (635 mg, 1.71 mmol, 1.2 eq) in THF (3 mL) at 0° C. N-bromosuccinimide (507 mg, 2.85 mmol, 2 eq) was added at RT and the reaction mixture was stirred 16 h before being quenched with ice-cold water and extracted with EtOAc. The organic layer was dried over anhydrous Na₂SO₄, filtered and concentrated in vacuo. The crude residue was purified by reverse phase column chromatography and the appropriate fractions were concentrated in vacuo to give tert-butyl 5-((4-(2,6-dimethoxy-4-(1,4,5-trimethyl-6-oxo-1,6-dihydropyridin-3-yl)benzyl)piperazin-1-yl)sulfonyl)-1-methyl-3,4-dihydroisoquinoline-2(1H)-carboxylate (500 mg, 0.22 mmol, 15.5% yield). LCMS m/z=681.2 [M+H]⁺.

459: By general procedure 2 in using HCl in 1,4-dioxane (4 M), in DCM. Obtained 120 mg. LCMS m/z=581.2 [M+H]⁺.

Compound C75: By general procedure 6. Obtained 43 mg, 32.7% yield. LCMS m/z=716.2 [M+H]⁺

### Overview of Synthetic Pathway for Following Structures Scheme 1

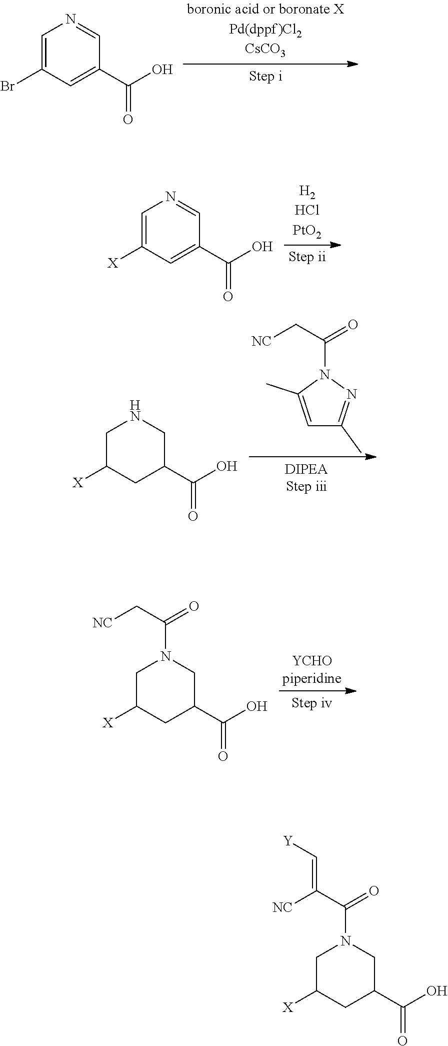

5-bromonicotinic acid Is treated with the desired boronate or boronic acid X under Suzuki conditions ((PdCl₂(dppf)·DCM, K₂CO₃, in dioxane/water at 100° C.) in order to obtain the 5 substituted nicotinic acids, unless commercially available. Hydrogenation under (H₂, PtO₂ in HCl or HOAc) affords the substituted nipecotic acids. Acylation using 1 cyanoacetyl-3,5-dimethylpyrazole and DIPEA In dioxane or DMF affords the precursors for the Knoevenagel reaction, which can be carried on using aldehydes Y in ethanol at r.t. (or THF at 40 to 70° C.) using piperidine as catalyst.

### 460: 5-phenylnicotinic Acid

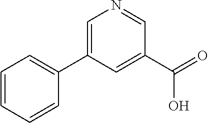 [SMILES: O=C(O)c1cncc(-c2ccccc2)c1]

To a stirred solution of 5-bromonicotinic acid (1.0 equiv., 2.0 g, 9.90 mmol), phenylboronic acid (1.2 equiv., 1.4 g, 11.88 mmol) in Dioxane (20 m) H₂O (4.0 ml) was added K₂CO₃ (2.0 equiv., 2.7 g, 19.80 mmol). After addition the reaction mixture was degassed with N₂ gas for 20 min and then added catalyst Pd(dppf)Cl₂·CH₂Cl₂ (0.1 equiv., 0.80 g, 0.990 mmol) at RT and then stirred at 100° C. for 16 h. The progress of the reaction was monitored by LCMS. The reaction mixture was filtered through a celite bed and washed with EtOAc and water. The filtrate was concentrated completely and added ice cold water was added. The mixture was extracted with EtOAc. The aqueous layer was acidified with 1.5 N HCl at 0° C. and stirred for 30 min until precipitation was observed. The precipitate was filtered off and washed with water and dried thoroughly to afford 5-phenylnicotinic acid (1.10 g, 5.41 mmol, 54.7% yield) as an off white solid. m/z=200.1 [M+H]⁺.

### ADDITIONAL EXAMPLES

Additional examples made in accordance with a method similar to that described above and illustrated in Scheme 1 are shown below.

| Example | | | m/z |
|---|---|---|---|
| number | Employed Boronate | Structure/Preparation | (M + H)⁺ |
| 461 | 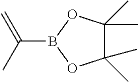 | 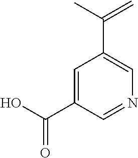 | 164.3 |
| 462 | 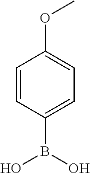 | 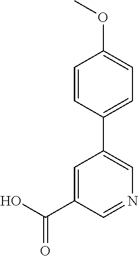 [SMILES: COc1ccc(-c2cncc(C(=O)O)c2)cc1] | 230.1 |
| 463 | 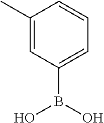 | 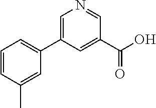 [SMILES: Cc1cccc(-c2cncc(C(=O)O)c2)c1] | 214.1 |

### Example 464: 5-phenylpiperidine-3-carboxylic Acid

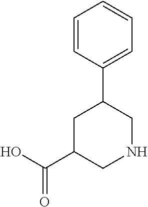 [SMILES: O=C(O)C1CNCC(c2ccccc2)C1]

To a stirred solution of 5-phenylnicotinic acid (1.0 equiv., 0.500 g, 2.510 mmol) in acetic acid (20 ml) was added Platinum(IV) oxide (0.4 equiv., 0.285 g, 1.255 mmol) at room temperature and stirred under hydrogen gas bladder pressure for 48 h. The progress of the reaction was monitored by LCMS. Reaction mass was filtered through celite bed and washed with methanol. The filtrate was concentrated under reduced pressure. The crude residue was purified by reverse phase column chromatography in 0.1% formic acid: acetonitrile to give 5-phenylpiperidine-3-carboxylic acid (0.500 g, 1.949 mmol, 99% yield) as gummy colourless compound. m/z=206.2 [M+H]⁺.

### ADDITIONAL EXAMPLES

Additional examples made in accordance with a method similar to that described above are shown below.

| Example | | m/z |
|---|---|---|
| number | Structure/Preparation | (M + H)⁺ |
| 465 | 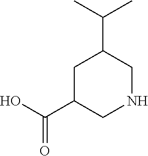 | 172.2 |
| 466 | 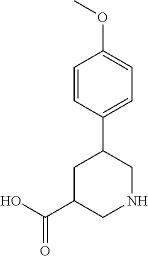 [SMILES: COc1ccc(C2CNCC(C(=O)O)C2)cc1] | 236.2 |
| 467 | 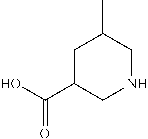 | 144.1 |

Compounds 465 to 467 and 3-piperidine carboxylic acid are then reacted with DIPEA and 1-cyanoacetyl-3,5-dimethyl-1H-pyrazole (as shown in Scheme 1).

### Example 468: 1-(2-cyanoacetyl)-6-phenylpiperidine-3-carboxylic Acid

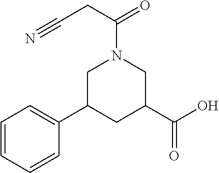 [SMILES: N#CCC(=O)N1CC(C(=O)O)CC(c2ccccc2)C1]

To a stirred solution of 5-phenylpiperidine-3-carboxylic acid (1.0 equiv, 0.600 g, 2.92 mmol in Dioxane (5.0 ml) were added DIPEA (5 equiv., 0.74 ml, 2.92 mmol) and 3-(3,5-dimethyl-1H-pyrazol-1-yl)-3-oxopropanenitrile (1.1 equiv., 0.525 g, 3.22 mmol) at RT. Reaction mixture stirred for 16 h at 90° C. The progress of the reaction was monitored by LCMS. Reaction mixture was concentrated and quenched with water (20 ml). The Aq. Layer was acidified with 1.5 N HCl and extracted with 10% MeOH:DCM (2×30 ml), Organic layer were dried over Na₂SO₄, filtered and concentrated. Crude product was purified by column chromatography (DCM/MeOH (4 to 7%)) to afford 1-(2-cyanoacetyl)-5-phenylpiperidine-3-carboxylic acid (0.230 g, 0.845 mmol, 28.9% yield) as gummy liquid. LCMS: m/z=[M−H]⁻ 271.0.

### ADDITIONAL EXAMPLES

| Example | | m/z |
|---|---|---|
| number | Structure/Preparation | (M + H)⁺ |
| 469 | 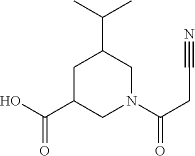 [SMILES: CC(C)C1CC(C(=O)O)CN(C(=O)CC#N)C1] | 238.1 |
| 470 | 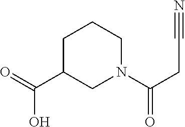 [SMILES: N#CCC(=O)N1CCCC(C(=O)O)C1] | 197.2 |
| 471 | 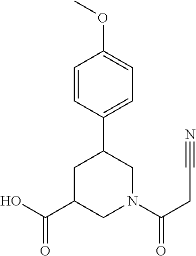 [SMILES: COc1ccc(C2CC(C(=O)O)CN(C(=O)CC#N)C2)cc1] | 303.1 |
| 472 | 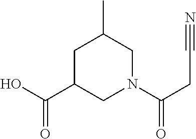 | 211.1 |

The resulting compounds are then reacted with the aldehydes shown below (as illustrated in Scheme 1).

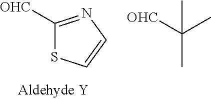

### Example 473: (E/Z)-1-(2-cyano-4,4-dimethylpent-2-enoyl)-5-phenylpiperidine-3-carboxylic Acid (Pipacid 0)

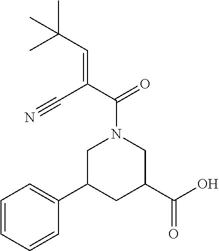 [SMILES: CC(C)(C)/C=C(\C#N)C(=O)N1CC(C(=O)O)CC(c2ccccc2)C1]

To a stirred solution of 1-(2-cyanoacetyl)-5-phenylpiperidine-3-carboxylic acid (0.070 g, 0.257 mmol in ethanol (2 ml) was added piperidine (0.044 g, 0.514 mmol) followed by addition of pivalaldehyde (0.033 g, 0.386 mmol) at RT under nitrogen atmosphere. The resulting reaction mixture was stirred at 60° C. for 16 h. Reaction was monitored by LCMS. The reaction was concentrated under reduced pressure to get a crude product. The crude product was acidified by adding 2N HCl in water and then product extracted in 10% MeOH/DCM (3×20 mL). Organic layer was dried over Na₂SO₄, filtered and concentrated under reduced pressure. The crude compound was purified by flash column chromatography (DCM/MeOH (0 to 10%)) to give (E)-1-(2-cyano-4,4-dimethylpent-2-enoyl)-5-phenylpiperidine-3-carboxylic acid (0.030 g, 0.060 mmol, 23.31% yield as a sticky oil. LCMS: m/z=[M−H]⁻¹ 339.4.

### ADDITIONAL EXAMPLES

| | | m/z |
|---|---|---|
| Name | Structure/Preparation | (M + H)⁺¹ |
| Pipacid 1 | 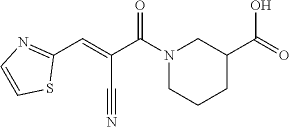 [SMILES: N#C/C(=C\c1nccs1)C(=O)N1CCCC(C(=O)O)C1] | 293.2 |
| Pipacid 2 | 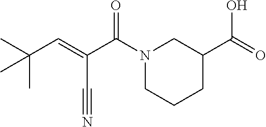 [SMILES: CC(C)(C)/C=C(\C#N)C(=O)N1CCCC(C(=O)O)C1] | 265.1 |
| Pipacid 3 | 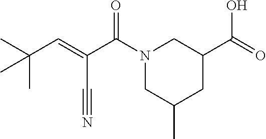 [SMILES: CC1CC(C(=O)O)CN(C(=O)/C(C#N)=C/C(C)(C)C)C1] | 279.3 |
| Pipacid 4 | 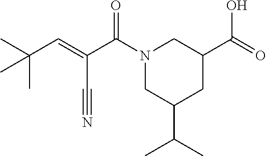 [SMILES: CC(C)C1CC(C(=O)O)CN(C(=O)/C(C#N)=C/C(C)(C)C)C1] | 307.1 |
| Pipacid 5 | 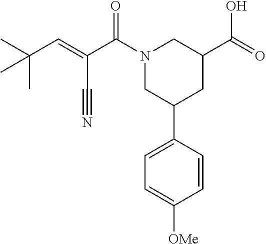 [SMILES: COc1ccc(C2CC(C(=O)O)CN(C(=O)/C(C#N)=C/C(C)(C)C)C2)cc1] | 371.3 |

### Overview of Synthetic Pathway—Scheme 1b

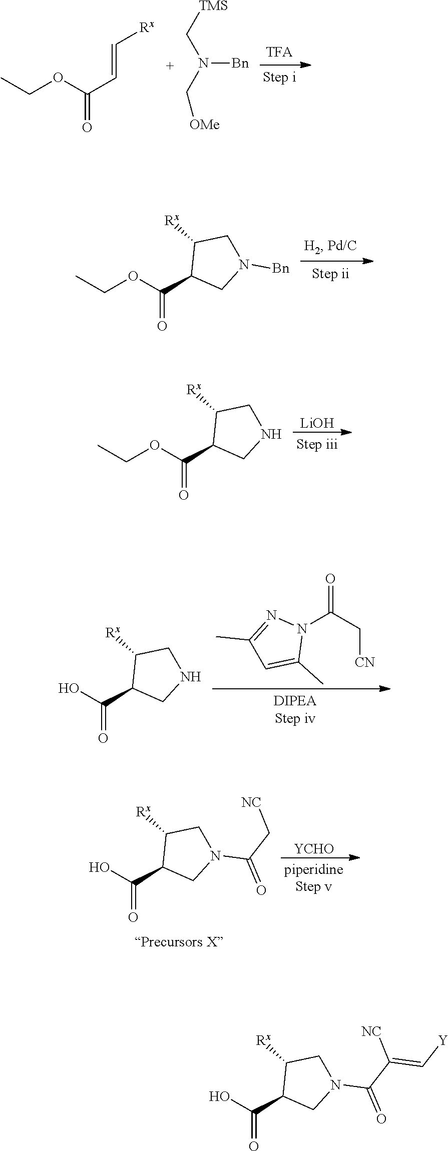

Acrylate esters are treated with N-benzyl-1-methoxy-N-((trimethylsilyl)methyl) methanamine and TFA in toluene to afford the trans-3,4-disubstituted N-benzyl-pyrrolidines. Cleavage of the benzyl group (H₂, Pd(OH)₂/C in ethanol or methanol) affords the free pyrrolidine analogues. Hydrolysis of the ester group (LiOH in THF/H₂O) affords the free amino acids. Acylation using 1 cyanoacetyl-3,5-dimethylpyrazole and DIPEA in dioxane or DMF affords the precursors X for the Knoevenagel reaction, which can be carried on using aldehydes Y shown below in ethanol at r.t. (or THF at 40 to 70° C.) using piperidine as catalyst.

Precursors X and aldehydes Y to be used in the Knoevenagel reaction (step v, Scheme 1b) are shown below.

| X | | Y |
|---|---|---|
| 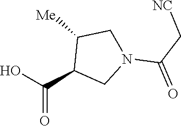 | 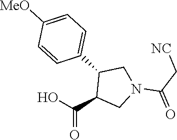 [SMILES: COc1ccc([C@@H]2CN(C(=O)CC#N)C[C@H]2C(=O)O)cc1] | 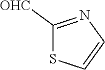 |
| 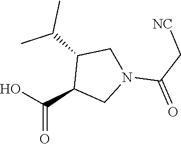 | 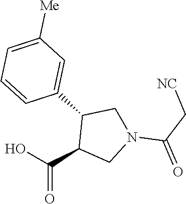 [SMILES: Cc1cccc([C@@H]2CN(C(=O)CC#N)C[C@H]2C(=O)O)c1] | 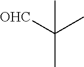 |
| 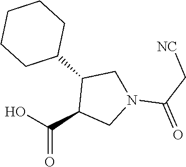 | 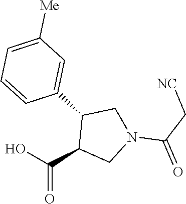 [SMILES: Cc1cccc([C@@H]2CN(C(=O)CC#N)C[C@H]2C(=O)O)c1] | 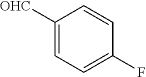 |

### Example 474: ethyl 1-benzyl-4-(m-tolyl)pyrrolidine-3-carboxylate

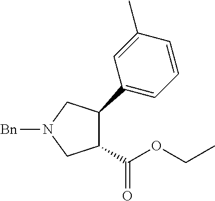 [SMILES: CCOC(=O)[C@@H]1CN(Cc2ccccc2)C[C@H]1c1cccc(C)c1]

To a stirred solution of ethyl (E)-3-(m-tolyl)acrylate (5.00 g, 26.3 mmol) in toluene (50 mL) was added N-benzyl-1-methoxy-N-((trimethylsilyl)methyl)methanamine (6.24 g, 26.3 mmol) at 25° C. The reaction mixture was cooled to 0° C. and then was added TFA (2.63 mL, 2.63 mmol) (1M in DCM) dropwise at 0° C. The reaction mixture was stirred for at 25° C. for 16 h under N₂ atmosphere. The progress of reaction mixture was monitored by TLC. The reaction mixture was diluted with water (50 mL), extracted with ethyl acetate (100 mL×2). Combined organic layer was washed with brine solution (50 mL), dried over Na₂SO₄ and filtered, concentrated under vacuum. The crude residue was purified by flash column chromatography, using 0 to 10% ethyl acetate in hexane to afford pure product ethyl 1-benzyl-4-(m-tolyl)pyrrolidine-3-carboxylate (8 g, 24.49 mmol, 93% yield) as a pale yellow liquid. LCMS: m/z=[M+H]⁺ 324.2.

### ADDITIONAL EXAMPLES

| Example | | m/z |
|---|---|---|
| number | Structure/Preparation | (M + H)⁺ |
| 475 | 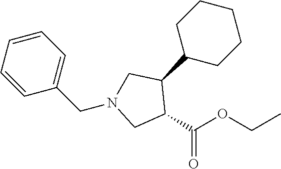 [SMILES: CCOC(=O)[C@@H]1CN(Cc2ccccc2)C[C@H]1C1CCCCC1] | 316.2 |
| 476 | 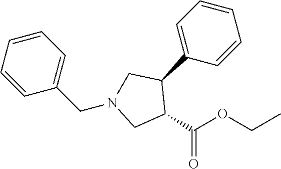 [SMILES: CCOC(=O)[C@@H]1CN(Cc2ccccc2)C[C@H]1c1ccccc1] | 311.2 |
| 477 | 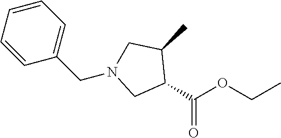 [SMILES: CCOC(=O)[C@@H]1CN(Cc2ccccc2)C[C@H]1C] | 248.1 |

### Example 478: Ethyl 4-(m-tolyl)pyrrolidine-3-carboxylate

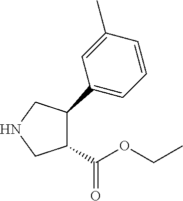 [SMILES: CCOC(=O)[C@@H]1CNC[C@H]1c1cccc(C)c1]

To a stirred solution of ethyl 1-benzyl-4-(m-tolyl)pyrrolidine-3-carboxylate (5.00 g, 15.5 mmol) in EtOH (40 mL) was added palladium hydroxide on carbon (1.085 g, 7.73 mmol) at 25° C. and the reaction was stirred for at 25° C. for 16 h under H₂ atmosphere. The progress of reaction mixture was monitored by TLC. The reaction mixture was filtered through celite pad and washed with ethanol, the filtrate was concentrated under vacuum to afford the product ethyl 4-(m-tolyl)pyrrolidine-3-carboxylate (3.5 g, 14.70 mmol, 95% yield) as a pale yellow liquid. LCMS: m/z=[M+H]⁺ 234.2.

### ADDITIONAL EXAMPLES

| Example | | m/z |
|---|---|---|
| number | Structure/Preparation | (M + H)⁺ |
| 479 | 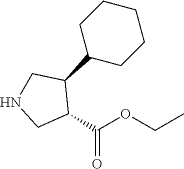 [SMILES: CCOC(=O)[C@@H]1CNC[C@H]1C1CCCCC1] | 226.2 |
| 480 | 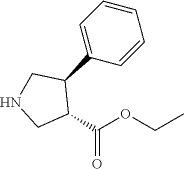 [SMILES: CCOC(=O)[C@@H]1CNC[C@H]1c1ccccc1] | 220.1 |
| 481 | 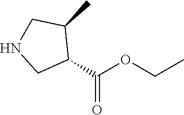 | 158.1 |

### Example 482: ethyl 1-(2-cyanoacetyl)-4-(m-tolyl)pyrrolidine-3-carboxylate

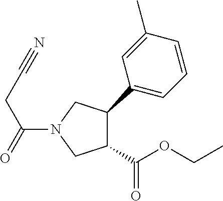 [SMILES: CCOC(=O)[C@@H]1CN(C(=O)CC#N)C[C@H]1c1cccc(C)c1]

To a stirred solution of ethyl 4-(m-tolyl)pyrrolidine-3-carboxylate (100 mg, 0.429 mmol) in Acetonitrile (2 mL) were added DIPEA (0.150 mL, 0.857 mmol), 3-(3,5-dimethyl-1H-pyrazol-1-yl)-3-oxopropanenitrile (84 mg, 0.514 mmol) at 25° C. The reaction mixture was stirred at 60° C. for 3 h under N₂ atmosphere. The progress of reaction mixture was monitored by TLC. The reaction mixture was diluted with water (2 mL), extracted with ethyl acetate (10 mL×2). Combined organic layer was dried over Na₂SO₄ and filtered, and concentrated under vacuum. The crude residue was purified by flash column using 0 to 20% ethyl acetate in hexanes to afford the product ethyl 1-(2-cyanoacetyl)-4-(m-tolyl)pyrrolidine-3-carboxylate (90 mg, 0.191 mmol, 44.7% yield) as a yellow semi-solid. LCMS: m/z-[M+H]⁺ 301.2.

### ADDITIONAL EXAMPLES

| Example | | m/z |
|---|---|---|
| number | Structure/Preparation | (M + H)⁺ |
| 483 | 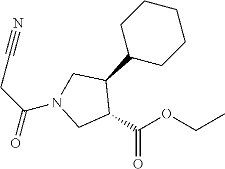 [SMILES: CCOC(=O)[C@@H]1CN(C(=O)CC#N)C[C@H]1C1CCCCC1] | 293.1 |
| 484 | 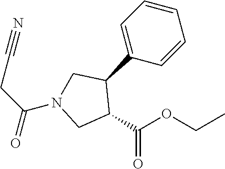 [SMILES: CCOC(=O)[C@@H]1CN(C(=O)CC#N)C[C@H]1c1ccccc1] | 287.1 |
| 485 | 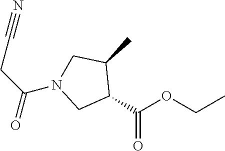 [SMILES: CCOC(=O)[C@@H]1CN(C(=O)CC#N)C[C@H]1C] | 225.1 |
| 486 | 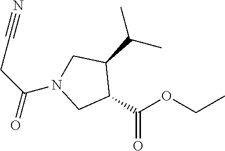 [SMILES: CCOC(=O)[C@@H]1CN(C(=O)CC#N)C[C@H]1C(C)C] | 253.2 |

### Example 487: 1-(2-cyanoacetyl)-4-(m-tolyl)pyrrolidine-3-carboxylic Acid

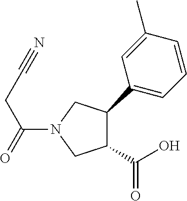 [SMILES: Cc1cccc([C@@H]2CN(C(=O)CC#N)C[C@H]2C(=O)O)c1]

To a stirred solution of ethyl (1-(2-cyanoacetyl)-4-(m-tolyl)pyrrolidine-3-carboxylate (2.3 g, 7.66 mmol) in MeOH (15 m), % War (15.00 ml) was added LiOH (0.275 g, 11.49 mmol) at 0° C. The reaction mixture was stirred at 25° C. for 16 h under N₂ atmosphere. The progress of reaction mixture was monitored by TLC. The reaction mixture was concentrated under vacuum and diluted with water, acidified with 1.5N HCl solution to pH=2. The resulting precipitate was filtered, washed with water and dried under vacuum to afford pure product (1-(2-cyanoacetyl)-4(m-tolyl)pyrrolidine-3-carboxylic acid (1 g, 3.67 mmol, 48.0% yield) as brown solid. LCMS: m/z=[M+H]⁺ 373.0.

### ADDITIONAL EXAMPLES

| Example | | m/z |
|---|---|---|
| number | Structure/Preparation | (M + H)⁺ |
| 488 | 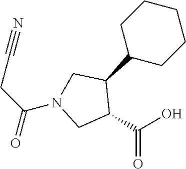 [SMILES: N#CCC(=O)N1C[C@@H](C(=O)O)[C@H](C2CCCCC2)C1] | 265.1 |
| 489 | 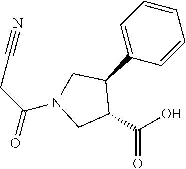 [SMILES: N#CCC(=O)N1C[C@@H](C(=O)O)[C@H](c2ccccc2)C1] | 259.1 |
| 490 | 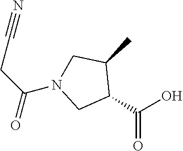 | 197.1 |
| 491 | 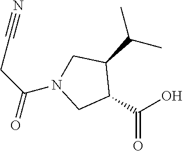 [SMILES: CC(C)[C@@H]1CN(C(=O)CC#N)C[C@H]1C(=O)O] | 225.2 |

### Example 492: 1-(E)-2-cyano-4,4-dimethylpent-2-enoyl)-4-(m-tolyl)pyrrolidine-4-carboxylic Acid

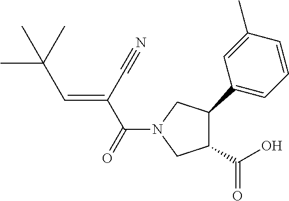 [SMILES: Cc1cccc([C@@H]2CN(C(=O)/C(C#N)=C/C(C)(C)C)C[C@H]2C(=O)O)c1]

To a stirred solution of 1-(2-cyanoacetyl)-4-(m-tolyl)pyrrolidine-3-carboxylic acid (100 mg, 0.387 mmol) in EtOH (2 ml-) were added piperidine (62.5 mg, 0.734 mmol), pivalaldehyde (95 mg, 1.102 mmol) at 25° C. the reaction mixture was stirred at 25° C. for 16 h under N₂ atmosphere. The progress of reaction mixture was monitored by TLC. The reaction mixture was concentrated to afford crude. The crude obtained was purified by flash column using 0 to 10% MeOH in DCM to afford product 1-((Z)-2-cyano-4,4-dimethylpent-2-enoyl)-4-(m-tolyl)pyrrolidine-3-carboxylic acid (27 mg, 0.021 mmol, 5.62% yield) as a brown semi solid. LCMS: m/z=[M+H]⁺ 341.1.

| Example | | m/z |
|---|---|---|
| number | Structure/Preparation | (M + H)⁺ |
| Pyracid 1 | 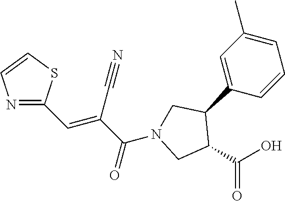 [SMILES: Cc1cccc([C@@H]2CN(C(=O)/C(C#N)=C/c3nccs3)C[C@H]2C(=O)O)c1] | 368.1 |
| Pyracid 2 | 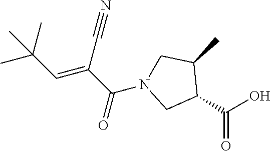 [SMILES: C[C@@H]1CN(C(=O)/C(C#N)=C/C(C)(C)C)C[C@H]1C(=O)O] | 265.1 |
| Pyracid 3 | 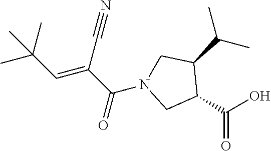 [SMILES: CC(C)[C@@H]1CN(C(=O)/C(C#N)=C/C(C)(C)C)C[C@H]1C(=O)O] | 293.1 |
| Pyracid 4 | 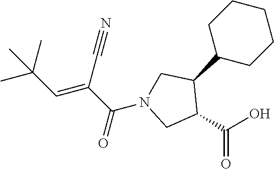 [SMILES: CC(C)(C)/C=C(\C#N)C(=O)N1C[C@@H](C(=O)O)[C@H](C2CCCCC2)C1] | 332.1 |
| Pyracid 5 | 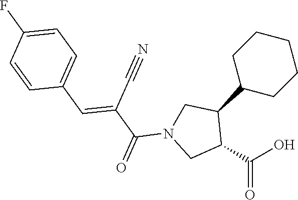 [SMILES: N#C/C(=C\c1ccc(F)cc1)C(=O)N1C[C@@H](C(=O)O)[C@H](C2CCCCC2)C1] | 371.1 |
| Pyracid 6 | 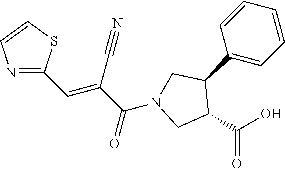 [SMILES: N#C/C(=C\c1nccs1)C(=O)N1C[C@@H](C(=O)O)[C@H](c2ccccc2)C1] | 354.1 |
| Pyracid 7 | 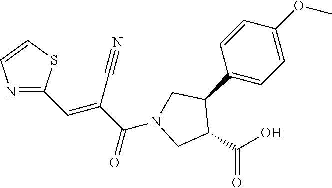 [SMILES: COc1ccc([C@@H]2CN(C(=O)/C(C#N)=C/c3nccs3)C[C@H]2C(=O)O)cc1] | 384.1 |

### Additional Exemplary Compounds

The following exemplary compounds were prepared by the following general procedure using 5-(3,5-dimethoxy-4-(piperazin-1-ylmethyl)phenyl)-1,3,4-trimethylpyridin-2(1H)-one synthesized in Table 2a and the appropriate acid selected from Pipacid 0 to 5 and Pyracid 0 to 7 as defined above.

### (E)-2-(3-(4-(2,6-dimethoxy-4-(1,4,5-trimethyl-6-oxo-1,6-dihydropyridin-3-yl)benzyl)piperazine-1-carbonyl)-6-methylpiperidine-1-carbonyl)-4,4-dimethylpent-2-enenitrile (B204)

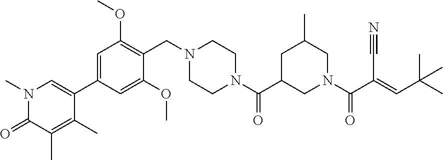 [SMILES: COc1cc(-c2cn(C)c(=O)c(C)c2C)cc(OC)c1CN1CCN(C(=O)C2CC(C)CN(C(=O)/C(C#N)=C/C(C)(C)C)C2)CC1]

A solution of 5-(3,5-dimethoxy-4-(piperazin-1-ylmethyl)phenyl)-1,3,4-trimethylpyridin-2(1H)-one (15.0 mg, 0.04 mmol) and (E)-1-(2-cyano-4,4-dimethylpent-2-enoyl)-5-methylpiperidine-3-carboxylic acid (11.2 mg, 0.04 mmol) in DMF (2 mL) were treated with HATU (18.4 mg, 0.05 mmol) and DIPEA (10.4 mg, 0.08 mmol). The reaction mixture was stirred at rt for 30 min, and LC-MS confirmed formation of the product mass. The solution was diluted with MeOH and purified by preparative HPLC (H₂O-MeCH (5-95%)+HCO₂H (0.01%)) to yield the desired product (E)-2-(3-(4-(2,6-dimethoxy-4-(1,4,5-trimethyl-6-oxo-1,6-dihydropyridin-3-yl)benzyl)piperazine-1-carbonyl)-5-methylpiperidine-1-carbonyl-4,4-dimethylpent-2-enenitrile (12.9 mg, 50.6%) as a colourless solid. LC-MS: 632.4 ([M+H]⁺).

### ADDITIONAL EXAMPLES

| Example | | m/z |
|---|---|---|
| number | Structure | (M + H) |
| B205 | 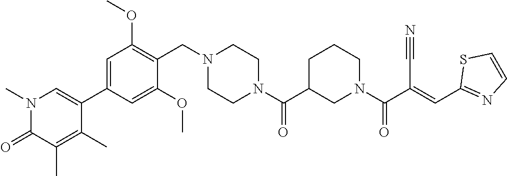 [SMILES: COc1cc(-c2cn(C)c(=O)c(C)c2C)cc(OC)c1CN1CCN(C(=O)C2CCCN(C(=O)/C(C#N)=C/c3nccs3)C2)CC1] | 645.3 |
| B206 | 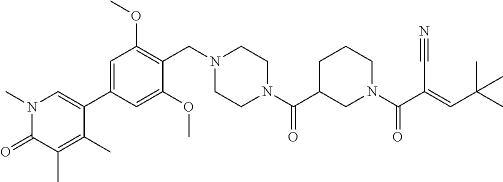 [SMILES: COc1cc(-c2cn(C)c(=O)c(C)c2C)cc(OC)c1CN1CCN(C(=O)C2CCCN(C(=O)/C(C#N)=C/C(C)(C)C)C2)CC1] | 618.4 |
| B207 | 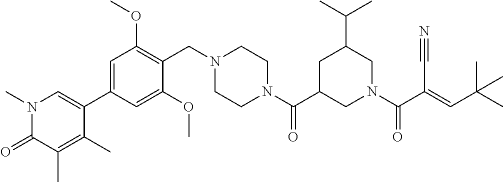 [SMILES: COc1cc(-c2cn(C)c(=O)c(C)c2C)cc(OC)c1CN1CCN(C(=O)C2CC(C(C)C)CN(C(=O)/C(C#N)=C/C(C)(C)C)C2)CC1] | 660.4 |
| B27 | 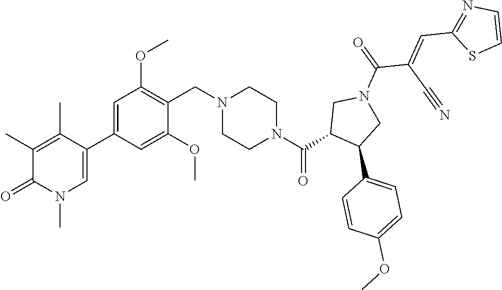 [SMILES: COc1ccc([C@@H]2CN(C(=O)/C(C#N)=C/c3nccs3)C[C@H]2C(=O)N2CCN(Cc3c(OC)cc(-c4cn(C)c(=O)c(C)c4C)cc3OC)CC2)cc1] | 737.3 |
| B208 | 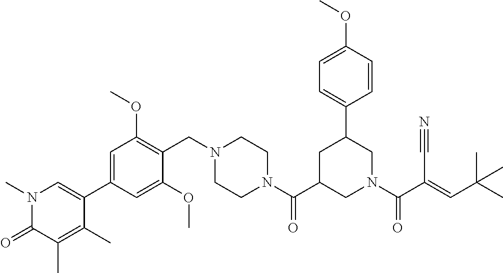 [SMILES: COc1ccc(C2CC(C(=O)N3CCN(Cc4c(OC)cc(-c5cn(C)c(=O)c(C)c5C)cc4OC)CC3)CN(C(=O)/C(C#N)=C/C(C)(C)C)C2)cc1] | 724.4 |
| B209 | 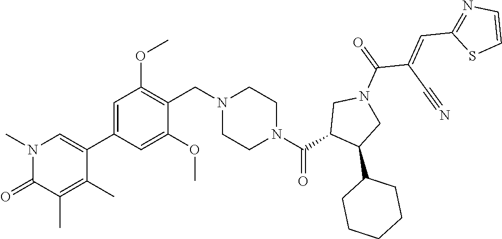 [SMILES: COc1cc(-c2cn(C)c(=O)c(C)c2C)cc(OC)c1CN1CCN(C(=O)[C@@H]2CN(C(=O)/C(C#N)=C/c3nccs3)C[C@H]2C2CCCCC2)CC1] | 713.4 |
| B210 | 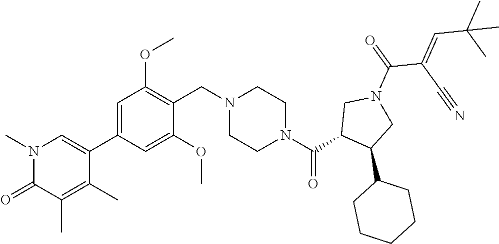 [SMILES: COc1cc(-c2cn(C)c(=O)c(C)c2C)cc(OC)c1CN1CCN(C(=O)[C@@H]2CN(C(=O)/C(C#N)=C/C(C)(C)C)C[C@H]2C2CCCCC2)CC1] | 686.4 |
| B211 | 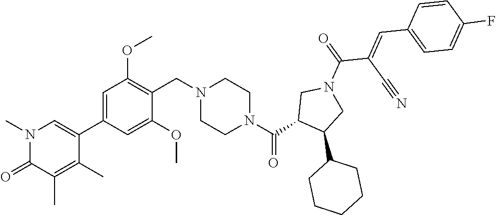 [SMILES: COc1cc(-c2cn(C)c(=O)c(C)c2C)cc(OC)c1CN1CCN(C(=O)[C@@H]2CN(C(=O)/C(C#N)=C/c3ccc(F)cc3)C[C@H]2C2CCCCC2)CC1] | 724.4 |
| B28 | 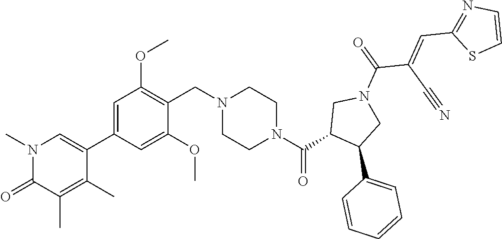 [SMILES: COc1cc(-c2cn(C)c(=O)c(C)c2C)cc(OC)c1CN1CCN(C(=O)[C@@H]2CN(C(=O)/C(C#N)=C/c3nccs3)C[C@H]2c2ccccc2)CC1] | 707.3 |
| B212 | 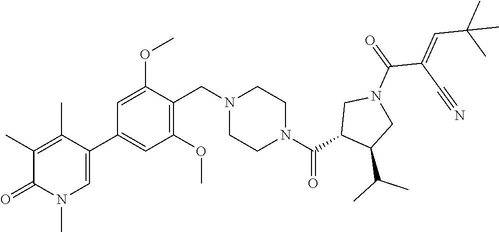 [SMILES: COc1cc(-c2cn(C)c(=O)c(C)c2C)cc(OC)c1CN1CCN(C(=O)[C@@H]2CN(C(=O)/C(C#N)=C/C(C)(C)C)C[C@H]2C(C)C)CC1] | 646.4 |
| B213 | 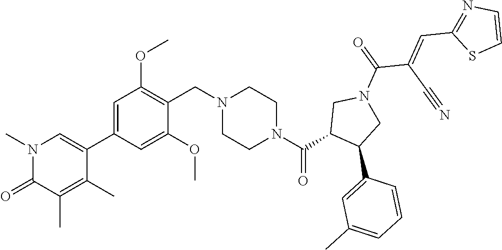 [SMILES: COc1cc(-c2cn(C)c(=O)c(C)c2C)cc(OC)c1CN1CCN(C(=O)[C@@H]2CN(C(=O)/C(C#N)=C/c3nccs3)C[C@H]2c2cccc(C)c2)CC1] | 721.3 |
| B214 | 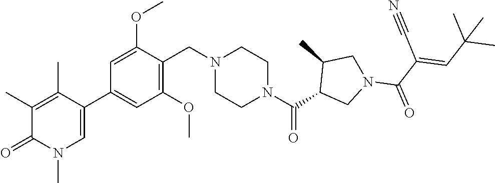 [SMILES: COc1cc(-c2cn(C)c(=O)c(C)c2C)cc(OC)c1CN1CCN(C(=O)[C@H]2CN(C(=O)/C(C#N)=C/C(C)(C)C)C[C@@H]2C)CC1] | 618.4 |

### 492: 3-fluoro-2,2-dimethyl Propanal

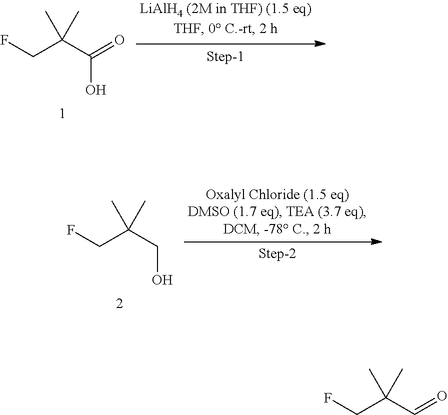

### 493: 3-fluoro-2,2-dimethylpropan-1-ol3-fluoro-2,2-dimethylpropan-1-ol

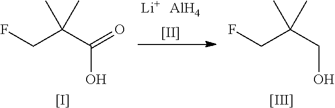

To a stirred solution of 3-fluoro-2,2-dimethylpropanoic acid (12 g, 100 mmol) in THF (100 mL), was added aluminum(III) lithium hydride (2M in THF) (49.9 ml, 100 mmol) at 0° C. The reaction mixture was stirred at 25° C. for 2 h. After completion of the reaction (TLC, monitoring in KMnO₄). The reaction mixture was quenched onto sodium sulphate as dropwise addition for 30 min under N₂ atm at 0° C. The organic layer was washed with water (100 mL×2), dried over anhydrous sodium sulphate filtered and concentrated under reduced pressure to yield 3-fluoro-2,2-dimethylpropan-1-ol-3-fluoro-2,2-dimethylpropan-1-ol (22 g, 207 mmol) as pale brown gum. (N.B. Compound volatile in nature. Concentration performed at lower temperature (25° C.).)

¹HNMR (DMSO-D6, 400 MHz): δ9.85 (s, 1H), 4.150 (t, 1H), 4.41 (s, 1H) 4.32 (s, 1H) 3.15 (m, 2H), 1.02 (s, 6H).

### 494: Synthesis of 3-fluoro-2,2-dimethyl Propanal

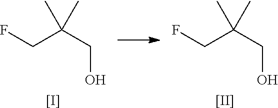

To a stirred solution of oxalyl chloride (21.77 mL, 249 mmol) in DCM (30 mL), at −78° C. was added DMSO (20.00 mL, 282 mmol) drop-wise, stirred for 10 min at same temperature. To the resultant reaction mixture was added 3-fluoro-2,2-dimethylpropan-1-ol (17.6 g, 166 mmol) in DCM, stirred for 20 min. Triethylamine (86 mL, 614 mmol) was added at −78° C., and the reaction mixture was stirred for 10 min. The reaction mixture was quenched with water (20 mL) and extracted with DCM (2×250 mL). The combined organic fractions were distilled by fractional distillation and volatile and DCM were removed at 35-40° C. The pure 3-fluoro-2,2-dimethyl propanal (12 g) was obtained as pale brown liquid.

¹HNMR (DMSO-D6, 400 MHz): δ9.65 (s, 1H), 4.150 (t, 1H), 4.41 (s, 1H) 4.32 (s, 1H) 3.15 (m, 2H), 1.02 (s, 6H).

### Compound C76 and Compound C77: Synthesis of Enantiomer 1 and Enantiomer 2 of (E/Z)-2-(5-((7-(2,6-dimethoxy-4-(1,4,5-trimethyl-6-oxo-1,6-dihydropyridin-3-yl)benzyl)-4,7-diazaspiro[2.5]octan-4-yl)methyl)-1-Isopropyl-1,2,3,4-tetrahydroisoquinoline-2-carbonyl)-5-fluoro-4,4-dimethylpent-2-enenitrile

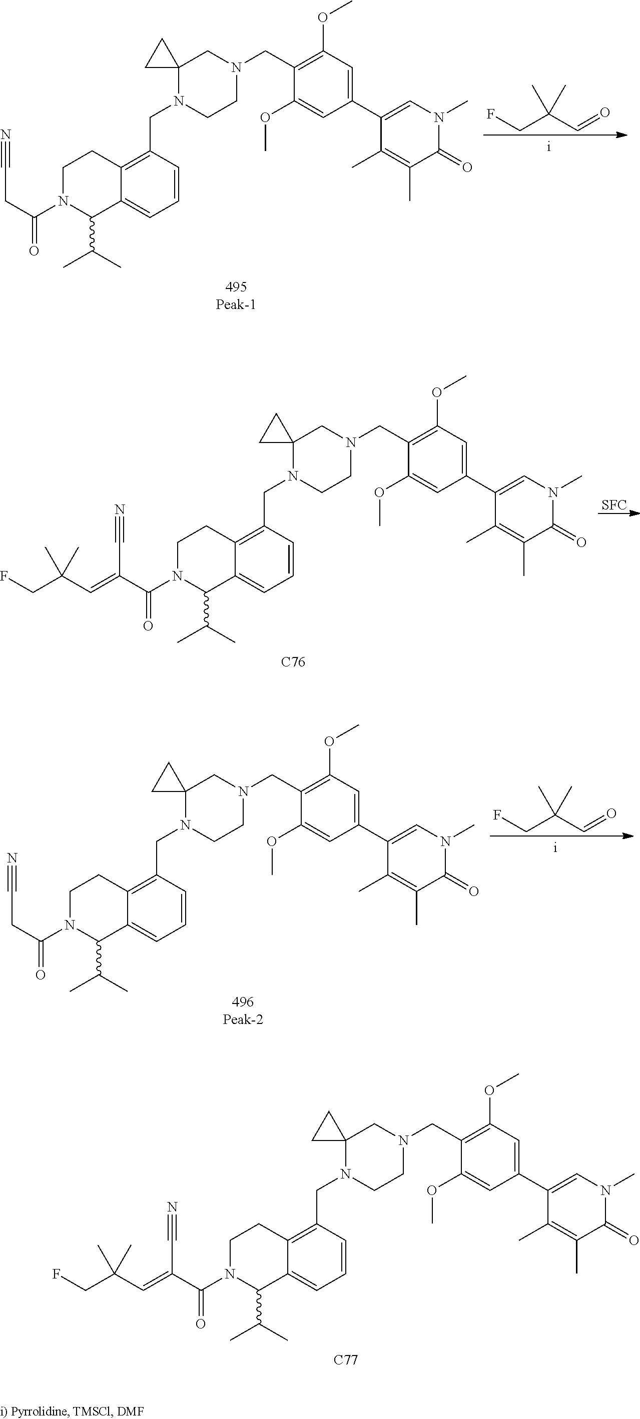

Racemic material (600 mg) was then purified by SFC:

| Column | RR WHELK-(250*30) mm, 5 μm | |
|---|---|---|
| Mobile Phase | CO₂: 0.5% isopropylamine in | |
| | MeOH:MeCN [55:45] | |
| Flow rate | 100 | mL/min |
| Back pressure | 100 | bar |
| Wavelength | 254 | nm |
| Cycle time | 4 | min |

Isolated Peak-1: 280 mg, 46% yield. LCMS m/z=652.4 [M+H]⁺ and Peak-2: 200 mg, LCMS m/z=652.4 [M+H]⁺

### Compound C76: Enantiomer 1 of (E/Z)-2-(5-((7-(2,6-dimethoxy-4-(1,4,5-trimethyl-6-oxo-1,6-dihydropyridin-3-yl)benzyl)-4,7-diazaspiro[2.5]octan-4-yl)methyl)-1-isopropyl-1,2,3,4-tetrahydroisoquinoline-2-carbonyl)-5-fluoro-4,4-dimethylpent-2-enenitrile

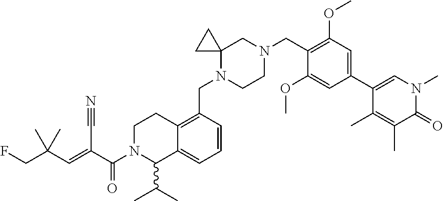 [SMILES: COc1cc(-c2cn(C)c(=O)c(C)c2C)cc(OC)c1CN1CCN(Cc2cccc3c2CCN(C(=O)/C(C#N)=C/C(C)(C)CF)C3C(C)C)C2(CC2)C1]

Prepared following general procedure 4 using peak 1 from the chiral SFC. Obtained 12 mg, 4.6% yield. LCMS m/z=738.4 [M+H]⁺.

### Compound C77: Enantiomer 2 of (E/Z)-2-(5-((7-(2,6-dimethoxy-4-(1,4,5-trimethyl-6-oxo-1,6-dihydropyridin-3-yl)benzyl)-4,7-diazaspiro[2.5]octan-4-yl)methyl)-1-isopropyl-1,2,3,4-tetrahydroisoquinoline-2-carbonyl)-5-fluoro-4,4-dimethylpent-2-enenitrile

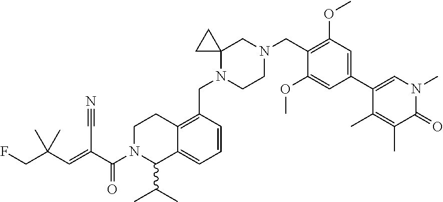 [SMILES: COc1cc(-c2cn(C)c(=O)c(C)c2C)cc(OC)c1CN1CCN(Cc2cccc3c2CCN(C(=O)/C(C#N)=C/C(C)(C)CF)C3C(C)C)C2(CC2)C1]

Prepared following general procedure 4 using peak 2 from the chiral SFC. Obtained 55 mg, 24.3% yield. LCMS m/z=738.4 [M+H]⁺.

### Enantiomer 1 and Enantiomer 2 of (E/Z)-2-(5-((7-(2,6-dimethoxy-4-(1,4,5-trimethyl-6-oxo-1,6-dihydropyridin-3-yl)benzyl)-4,7-diazaspiro[2.5]octan-4-yl)methyl)-1-isopropyl-1,2,3,4-tetrahydroisoquinoline-2-carbonyl)-4,4-dimethylpent-2-enenitrile (C78 and C79)

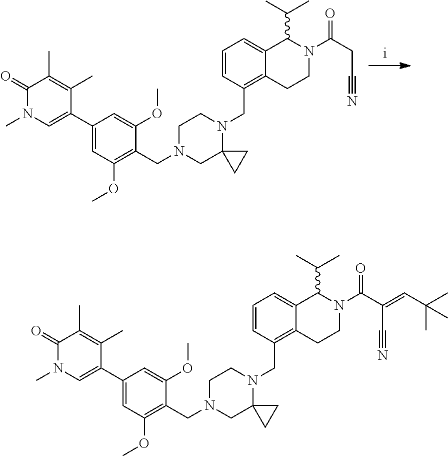 [SMILES: COc1cc(-c2cn(C)c(=O)c(C)c2C)cc(OC)c1CN1CCN(Cc2cccc3c2CCN(C(=O)CC#N)C3C(C)C)C2(CC2)C1 ; COc1cc(-c2cn(C)c(=O)c(C)c2C)cc(OC)c1CN1CCN(Cc2cccc3c2CCN(C(=O)/C(C#N)=C/C(C)(C)C)C3C(C)C)C2(CC2)C1]

Compound C78: Synthesis of Enantiomer 2: By general procedure 4 using peak 2 from the chiral SFC. Obtained 19 mg, 18% yield. LCMS m/z=720.3 [M+H]+.

Compound C79: Synthesis of enantiomer 1: By general procedure 4 using peak 1 from the chiral SFC. Obtained 17 mg, 15% yield. LCMS m/z=720.4 [M+H]+.

### (E/Z)-2-(5-((1-(2,6-dimethoxy-4-(1,4,5-trimethyl-6-oxo-1,6-dihydropyridin-3-yl)benzyl)piperidin-4-yl)methyl)-1-isopropyl-1,2,3,4-tetrahydroisoquinoline-2-carbonyl)-8-fluoro-4,4-dimethylpent-2-enenitrile (C80)

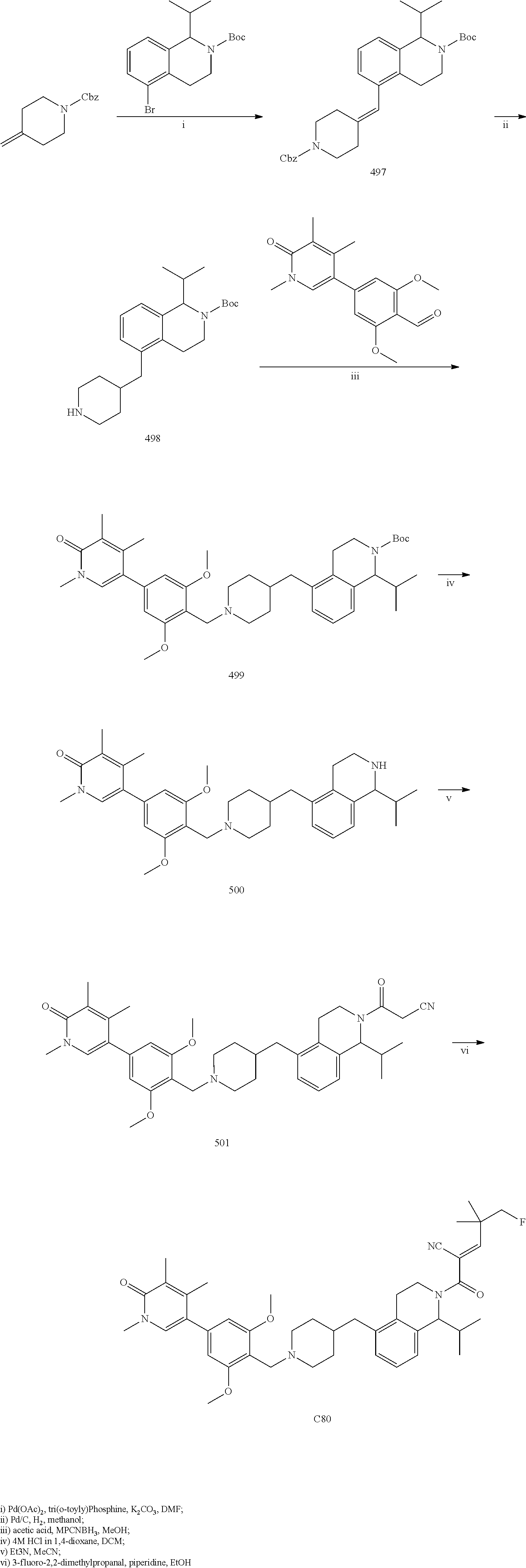

497: Prepared by following general procedure 31. Obtained 490 mg, 44% yield. LCMS m/z=405.2 [M+H−Boc]⁺.

498: Prepared by following general procedure 27. Obtained 298 mg, 82% yield. LCMS m/z=373.4 [M+H−Boc]⁺.

499: Prepared by following general procedure 1 using MPCNBH₃ (w/w) in MeOH. Obtained 220 mg, 47.1% yield. LCMS m/z=658.4 [M+H]⁺.

500: Prepared by following general procedure 2 using 4M HCl in 1,4-dioxane (1 eq) in DCM. Obtained 200 mg (crude). LCMS m/z=558.3 [M+H]⁺.

501: Prepared by following general procedure 3. Obtained 120 mg, 44.9% yield. LCMS m/z=625.3 [M+H]⁺. Compound C80: Prepared by following general procedure 4. Obtained 12.2 mg, 17.2% yield. LCMS m/z=711.3 [M+H]⁺.

### Compound C81 and Compound C82: Single Enantiomer of (E/Z)-2-(5-((4-(2,6-dimethoxy-4-(1,4,5-trimethyl-6-oxo-1,6-dihydropyridin-3-yl)benzyl)-4,7-diazaspiro[2.5]octan-7-yl)methyl)-1-isopropyl-1,2,3,4-tetrahydroisoquinoline-2-carbonyl)-4,4-dimethylpent-2-enenitrile

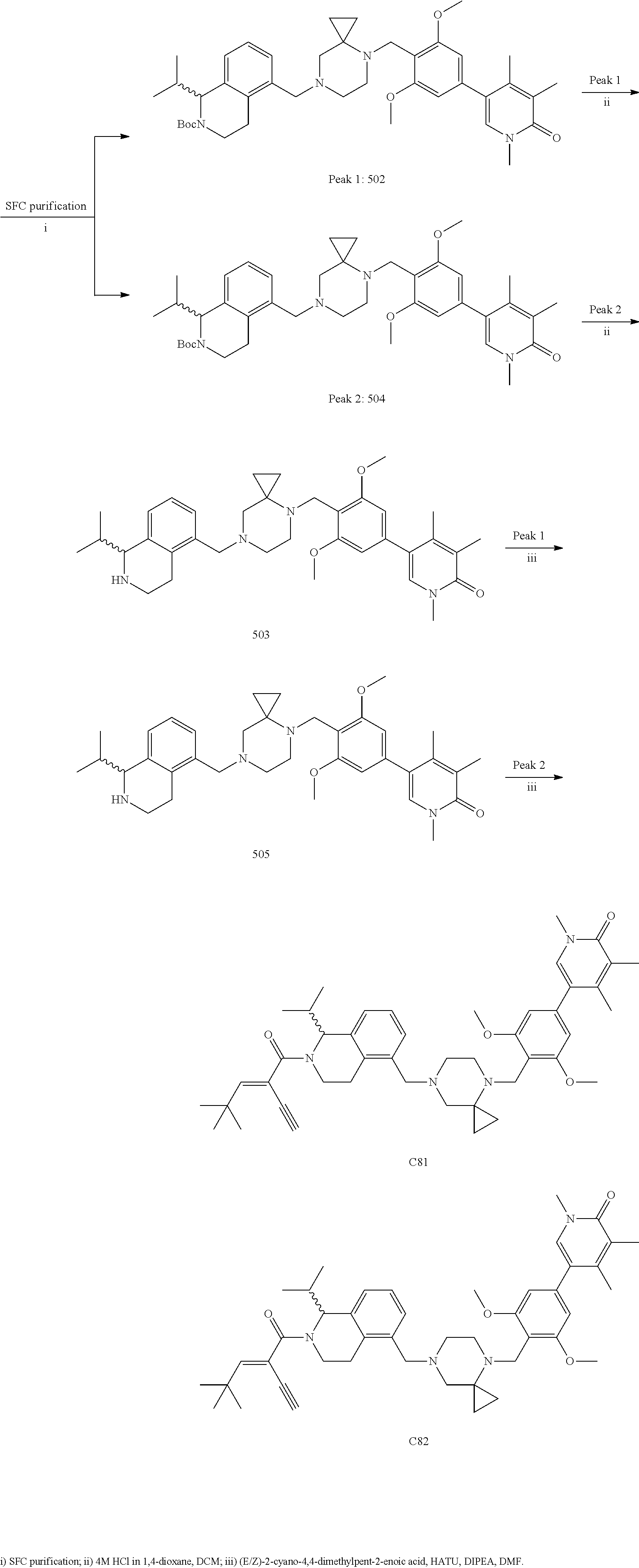

### Enantiomer 1 and 2 of tert-butyl 5-((4-(2,6-dimethoxy-4-(1,4,5-trimethyl-6-oxo-1,6-dihydropyridin-3-yl)benzyl)-4,7-diazaspiro[2.5]octan-7-yl)methyl)-1-isopropyl-3,4-dihydroisoquinoline-2(1H)-carboxylate (502 and 504)

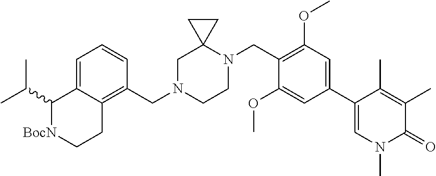 [SMILES: COc1cc(-c2cn(C)c(=O)c(C)c2C)cc(OC)c1CN1CCN(Cc2cccc3c2CCN(C(=O)OC(C)(C)C)C3C(C)C)CC12CC2]

Racemic material (480 mg) was then purified by SFC:

| Column | I CELLULOSE Z-(250*30)mm, 5 μm | |
|---|---|---|
| Mobile Phase | CO₂: 0.5% isopropylamine in | |
| | MeOH:MeCN [65:35] | |
| Flow rate | 100 | mL/min |
| Back pressure | 100 | bar |
| Wavelength | 220 | nm |
| Cycle time | 6.5 | min |

Isolated Peak-1: 190 mg, LCMS m/z=685.4 [M+H]⁺ and Peak-2: 150 mg, LCMS M/z=685.4 [M+H]⁺

503:

Prepared by following general procedure 2. Obtained 138 mg (crude). LCMS m/z=585.4 [M+H]⁺.

### Compound C81:

Prepared by following general procedure 6. Obtained 53 mg, 41% yield. LCMS m/z=720.3 [M+H]⁺.

505:

Prepared by following general procedure 2 using 4M solution of HCl in 1,4-dioxane, in DCM. Obtained 125 mg, 98% yield. LCMS m/z=585.4 [M+H]⁺.

### Compound C82:

Prepared by following general procedure S. Obtained 53 mg, 41% yield. LCMS m/z=720.3 [M+H]⁺.

### Compound C83 (Peak 1) and Compound C84 (Peak 2)

### Synthesis of Both Enantiomers of (E/Z)-2-(5-((4-(2,6-dimethoxy-4-(1,4,5-trimethyl-6-oxo-1,6-dihydropyridin-3-yl)benzyl)-1,4-diazepan-1-yl)methyl)-1-isopropyl-1,2,3,4-tetrahydroisoquinoline-2-carbonyl)-5-fluoro-4-dimethylpent-2-enenitrile

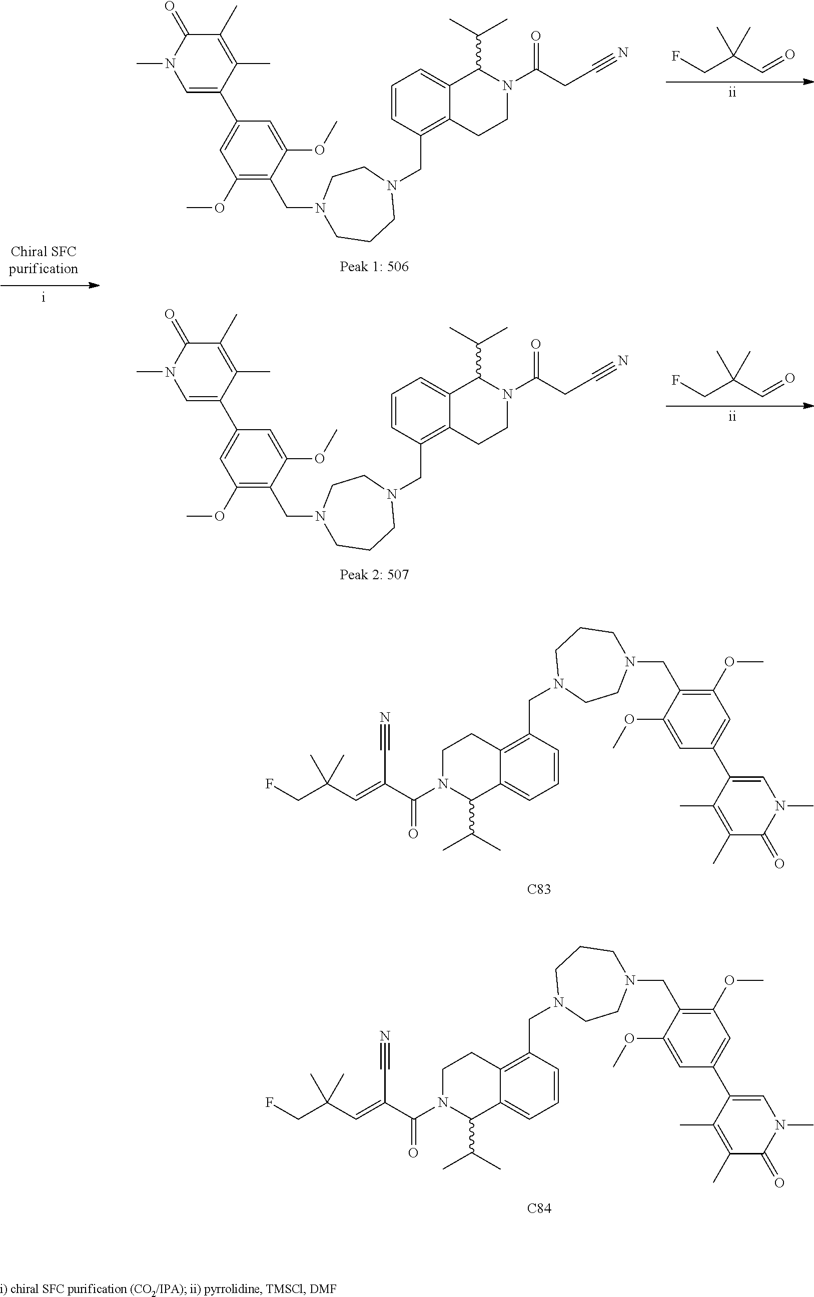

Racemic material (1 g) was then purified by SFC:

| Instrument | PIC 22-028 | |
|---|---|---|
| Column | WHELK RR-(250*20)mm, 5 μm | |
| Mobile Phase | CO₂: 0.5% isopropylamine in | |
| | MeOH:MeCN [60:40] | |
| Flow rate | 50 | mL/min |
| Back pressure | 120 | bar |
| Wavelength | 254 | nm |
| Cycle time | 7 | min |

Isolated Peak-1 (506): 350 mg, 34.3% yield. LCMS m/z=640.2 [M+H]⁺ and Peak-2 (507): 380 mg, 36.1% yield. LCMS m/z=640.2 [M+H]⁺

### Compound C83 (Peak 1):

Prepared following general procedure 4 and Peak 1 from SFC purification. Obtained 63 mg, 35.5% yield. LCMS m/z=726.4 [M+H]⁺.

### Compound C84 (Peak 2):

Prepared following general procedure 4 and Peak 2 from SFC purification. Obtained 44 mg, 25.3% yield. LCMS m/z=726.4 [M+H]⁺.

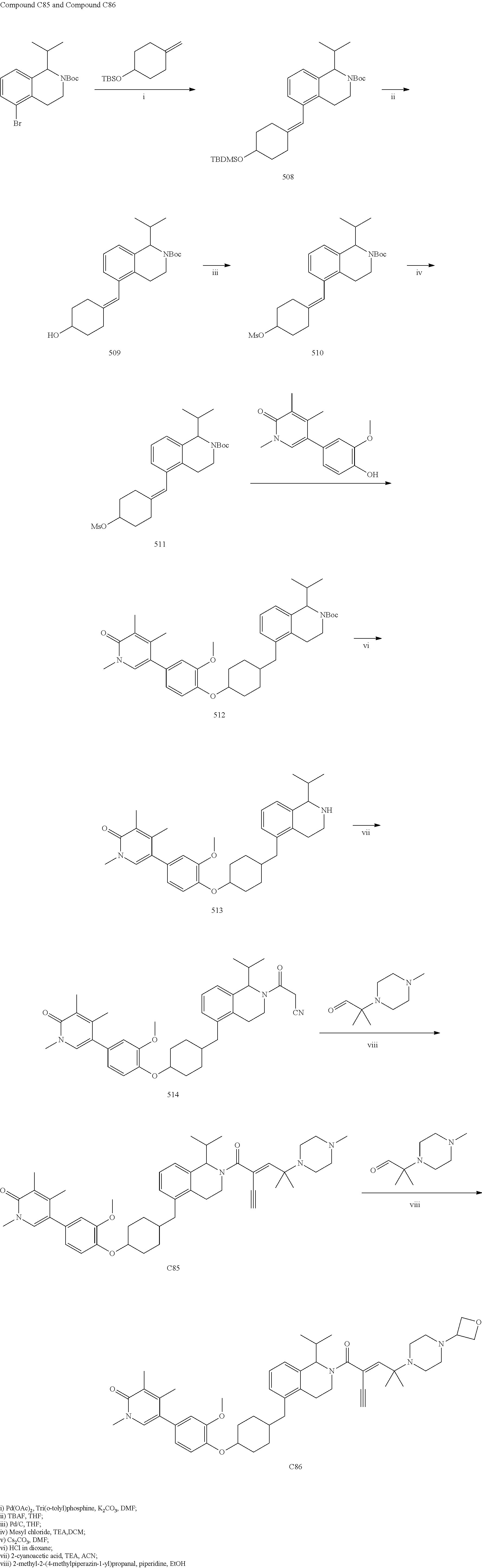

508: Prepared following general procedure 31. Obtained 2 g, 75% yield. LCMS m/z=445.6 [M−56]⁺.

509: To a stirred solution of tert-butyl 5-((4-((tert-butyldimethylsilyl)oxy)cyclohexylidene)methyl)-1-isopropyl-3,4-dihydroisoquinoline-2(1H)-carboxylate (2.0 g, 4.0 mmol, 1 eq) was added TBAF in THF (1 M, 8 mL, 8.0 mmol, 2 eq) was added at 0° C. The reaction mixture was stirred at RT for 8 h before being concentrated in vacuo. The resulting residue was purified by silica gel column chromatography (gradient=0-20% EtOAc in hexane). The appropriate fractions were concentrated in vacuo to yield tert-butyl 5-((4-hydroxycyclohexylidene)methyl)-1-isopropyl-3,4-dihydroisoquinoline-2(1H)-carboxylate (1.2 g, 81% yield). LCMS m/z=286.4 [M-Boc]⁺.

510: To a degassed solution of tert-butyl 5-((4-hydroxycyclohexylidene)methy)-1-isopropyl-3,4-dihydroisoquinoline-2(1H)-carboxylate (1.2 g, 3.11 mmol) in THF (30 mL) at RT was added Pd/C (1.325 g, 1.245 mmol, 0.4 eq) and the reaction mixture was stirred under hydrogen atmosphere for 16 h. Upon completion, the reaction mixture was filtered through celite, the celite was washed with EtOAc and the filtrate was concentrated in vacuo. The product was used in the next step without further purification. LCMS m/z=288.4 [M−Boc]⁺.

511: Prepared following general procedure 25. Obtained 500 mg, 78% yield.

512: Prepared following general procedure 26. Obtained 500 mg, 78% yield. LCMS m/z=629.7 [M+H]⁺.

513: Prepared following general procedure 2. Obtained 156 mg. LCMS m/z=529.3 [M+H]⁺.

514: Prepared following general procedure 3. Obtained 110 mg, 58% yield. LCMS m/z=596.2 [M+H]⁺.

Compound C85: Prepared following general procedure 4. Obtained 110 mg, 58% yield. LCMS m/z=748.4 [M+H]⁺.

Compound C86: Prepared following general procedure 4. Obtained 100 mg, 52% yield. LCMS m/z=790.4 [M+H]⁺.

### Compound C87 and Compound C88 Enantiomer 1 and Enantiomer 2 of (E/Z)-2-(7-((7-(2,6-dimethoxy-4-(1,4,5-trimethyl-6-oxo-1,6-dihydropyridin-3-yl)benzyl)-4,7-diazaspiro[2.5]octan-4-yl)methyl)-1-isopropyl-1,2,3,4-tetrahydroisoquinoline-2-carbonyl)-4,4-dimethylpent-2-enenitrile

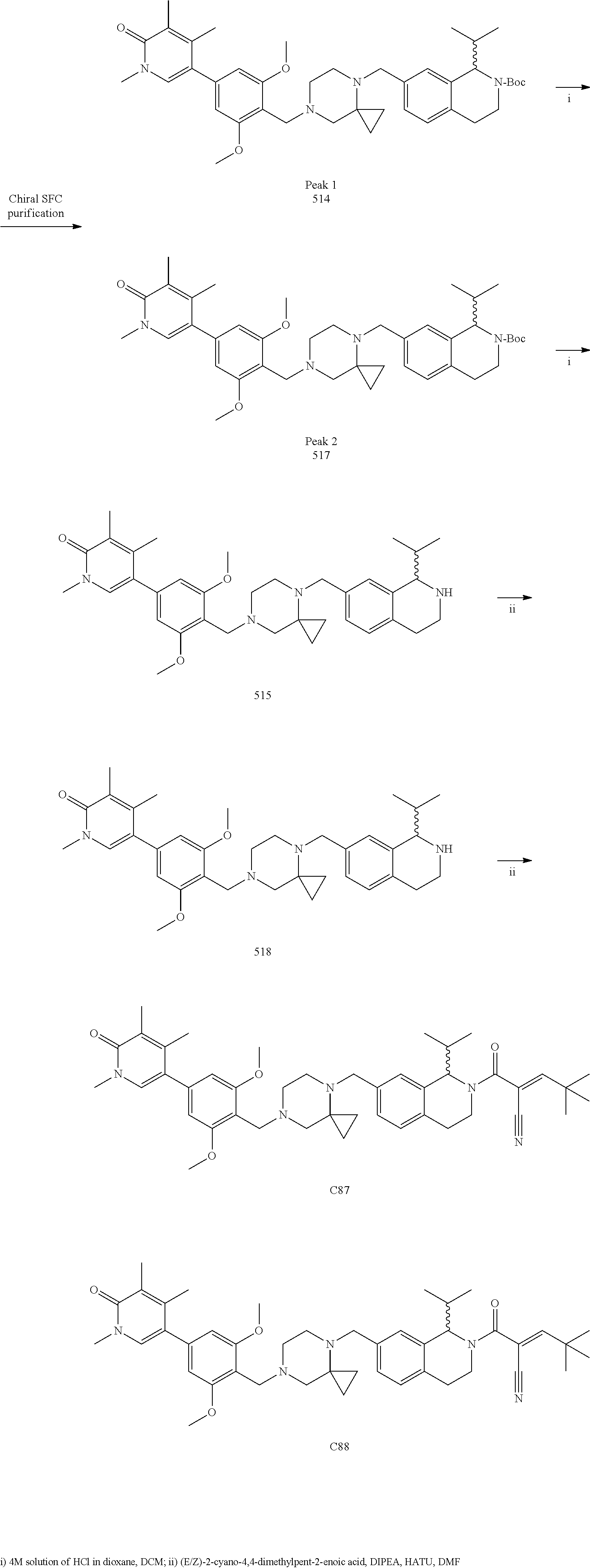

Racemic material (600 mg) was purified by SFC:

| Column | WHELK RR-(250*20)mm, 5 μm | |
|---|---|---|
| Mobile Phase | CO₂: 0.5% isopropylamine in | |
| | MeOH:MeCN [65:35] | |
| Flow rate | 4 | mL/min |
| Back pressure | 100 | bar |
| Wavelength | 254 | nm |
| Cycle time | 11 | min |

Isolated Peak-1 (514): 250 mg, LCMS m/z=685.3 [M+H]⁺ and Peak-2 (517): 240 mg, LCMS m/z=685.4 [M+H]³⁰

515: Prepared following general procedure 2. Obtained 230 mg. LCMS m/z=585.3 [M+H]⁺.

Compound C87: Prepared following general procedure 6. Obtained 10 mg, 20% yield. LCMS m/z=720.4 [M+H]⁺.

517: Prepared following general procedure 2. Obtained 205 mg. LCMS m/z=585.3 [M+H]⁺.

Compound C88: Prepared following general procedure 6. Obtained 6 mg, 10% yield. LCMS m/z=720.4 [M+H]⁺.

### Compound C89

### Single Enantiomer of (E/Z)-2-(7-(((2,6-dimethoxy-4-(1,4,5-trimethyl-6-oxo-1,6-dihydropyridin-3-yl)benzyl)methyl)amino)methyl)-1-isopropyl-1,2,3,4-tetrahydroisoquinoline-2-carbonyl)-4,4-dimethylpent-2-enenitrile

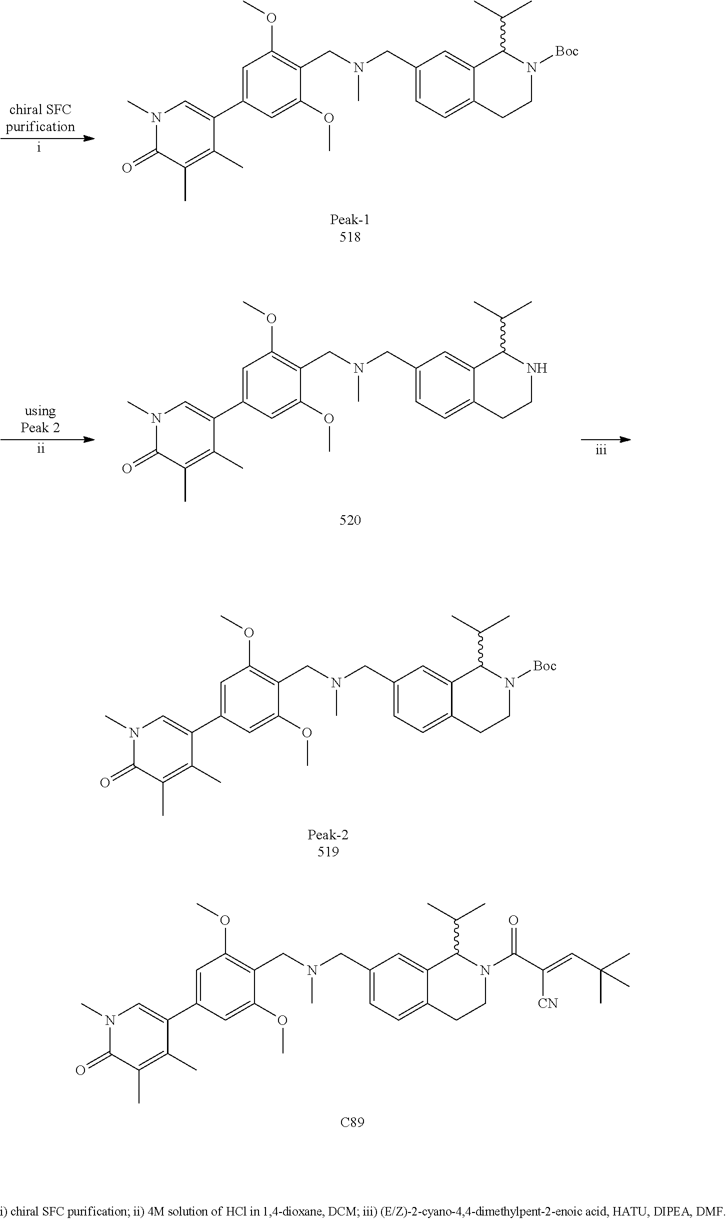

Racemic material (680 mg) was purified by SFC:

| Column | WHELK RR-(250*20)mm, 5 μm | |
|---|---|---|
| Mobile Phase | CO₂: 0.5% isopropylamine in | |
| | MeOH:MeCN [60:40] | |
| Flow rate | 50 | mL/min |
| Back pressure | 120 | bar |
| Wavelength | 254 | nm |
| Cycle time | 7 | min |

Isolated Peak-1 (518): 260 mg, LCMS m/z=604.0 [M+H]⁺ and Peak-2 (519): 140 mg, LCMS m/z=604.0 [M+H]⁺

520: Prepared by following general procedure 2 using 4M in dioxane HCl. Obtained 110 mg (crude). LCMS m/z=504.2 [M+H]⁺.

Compound C89: Prepared by following general procedure 6. Obtained 39 mg, 13% yield. LCMS m/z=639.6 [M+H]⁺.

### Compound C90 (Peak 1) and Compound C91 (Peak 2)

### Enantiomer 1 and Enantiomer 2 of (E/Z)-2-(5-((4-(2,6-dimethoxy-4-(1,4,5-trimethyl-6-oxo-1,6-dihydropyridin-3-yl)benzyl)-4,7-diazaspiro[2.5]octan-7-yl)methyl)-1-ethyl-1,2,3,4-tetrahydroisoquinoline-2-carbonyl)-4,4-dimethylpent-2-enenitrile

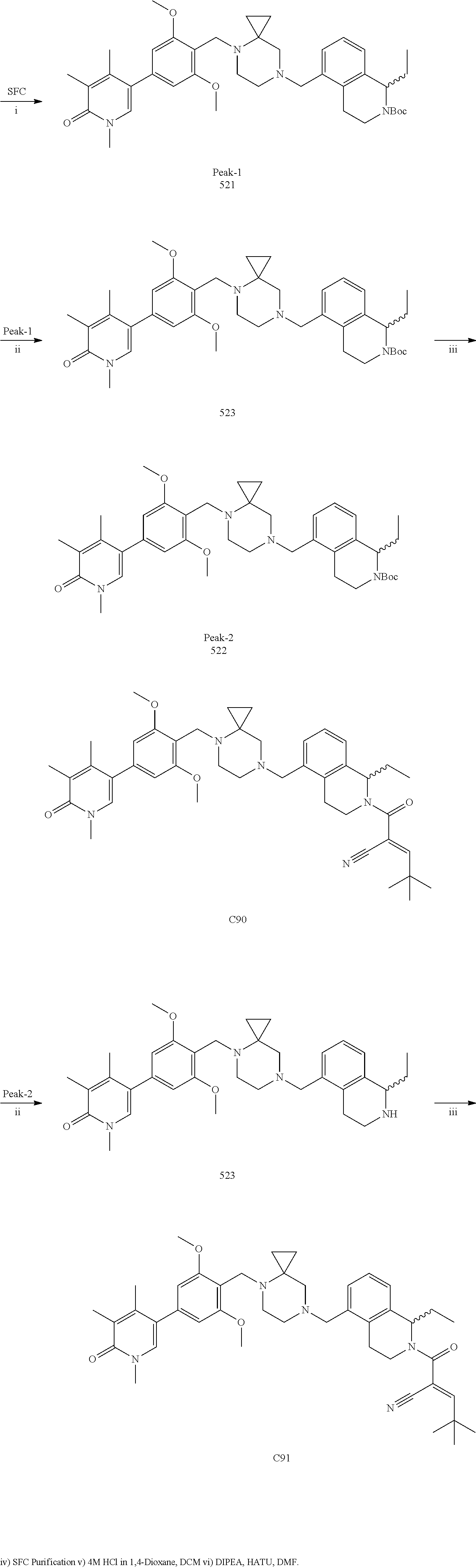

Racemic material (1.8 g) was purified b asymmetric SFC:

| Column | I Cellulose B- (250*30)mm, 5 μm | |
|---|---|---|
| Mobile Phase | CO₂: 0.5% isopropylamine in | |
| | MeOH:MeCN [85:15] | |
| Flow rate | 120 | mL/min |
| Back pressure | 120 | bar |
| Wavelength | 254 | nm |
| Cycle time | 10 | min |

Isolated Peak-1 (521): 650 mg, LCMS m/z=671.4 [M+H]⁺ and Peak-2 (522): 540 mg, LCMS m/z=671.4 [M+H]⁺

523: Prepared following general procedure 2 using HCl (4M solution in 1,4-dioxane), in DCM. Obtained 580 mg, LCMS m/z=−571.2[M+H]⁺.

Compound C90: Prepared by following general procedure 6. Obtained 24 mg, 17.3% yield. LCMS m/z=706.3 [M+H]⁺

524: Prepared following general procedure 2 using HCl (4M solution in 1,4-dioxane), in DCM. Obtained 450 mg, LCMS m/z=571.3 [M+H]⁺.

Compound C91: Prepared by following general procedure 6. Obtained 64 mg, 45.4% yield. LCMS m/z=706.3 [M+H]⁺.

### Compound C92 (Peak 1) and Compound C93 (Peak 2)

### Enantiomer 1 and Enantiomer 2 of (E/Z)-2-(5-((7-(2,6-dimethoxy-4-(1,4,5-trimethyl-6-oxo-1,6-dihydropyridin-3-yl)benzyl)-4,7-diazaspiro[2.5]octan-4-yl)methyl)-1-ethyl-1,2,3,4-tetrahydroisoquinoline-2-carbonyl)-5-fluoro-4,4-dimethylpent-2-enenitrile

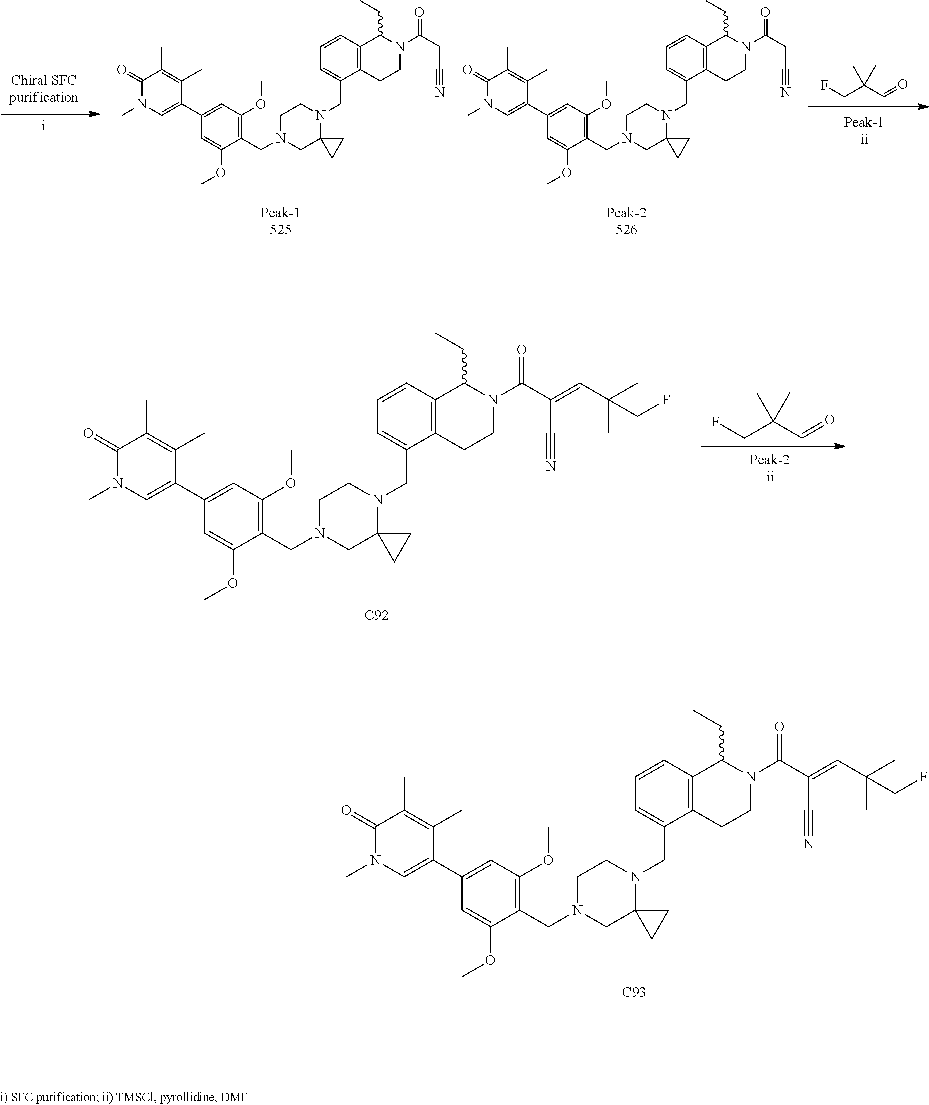

Racemic material (390 mg) was purified by asymmetric SFC:

| Column | WHELK RR-(250*20)mm, 5 μm | |
|---|---|---|
| Mobile Phase | CO₂: 0.5% isopropylamine in | |
| | MeOH:MeCN [50:50] | |
| Flow rate | 4 | mL/min |
| Back pressure | 100 | bar |
| Wavelength | 254 | nm |
| Cycle time | 10 | min |

Isolated Peak-1 (525): 140 mg, LCMS m/z=838.4 [M+H]⁺ and Peak-2 (526: 150 mg, LCMS m/z=638.4 [M+H]⁺

Compound C92: Prepared by following general procedure 4 using peak 1 from SFC above. Obtained: 36 mg, 46.8% yield. LCMS m/z=724.4 [M+H]⁺

Compound C93: Prepared by following general procedure 4 using peak 1 from SFC above. Obtained 53 mg, 82.5% yield. LCMS m/z=724.4 [M+H]⁺.

### Compound C94: (E/Z)-2-(5-((7-(2,6-dimethoxy-4-(1,4,5-trimethyl-6-oxo-1,6-dihydropyridin-3-yl)benzyl)-4,7-diazaspiro[2.5]octan-4-yl)methyl)-1-ethyl-1,2,3,4-tetrahydroisoquinoline-2-carbonyl)-4,4-dimethylpent-2-enenitrile

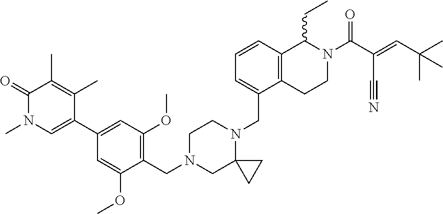 [SMILES: CCC1c2cccc(CN3CCN(Cc4c(OC)cc(-c5cn(C)c(=O)c(C)c5C)cc4OC)CC34CC4)c2CCN1C(=O)/C(C#N)=C/C(C)(C)C]

Prepared by following general procedure 6 using peak 1 from the SFC above (525). Obtained 31.8 mg, 39.6% yield. LCMS m/z=706.4 [M+H]

### Compound C95: (E/Z)-2-(5-(((2,6-dimethoxy-4-(1,4,5-trimethyl-6-oxo-1,6-dihydropyridin-3-yl)benzyl)(methyl)amino)methyl)-1-isopropyl-1,2,3,4-tetrahydroisoquinoline-2-carbonyl)-4,4-dimethylpent-2-enenitrile

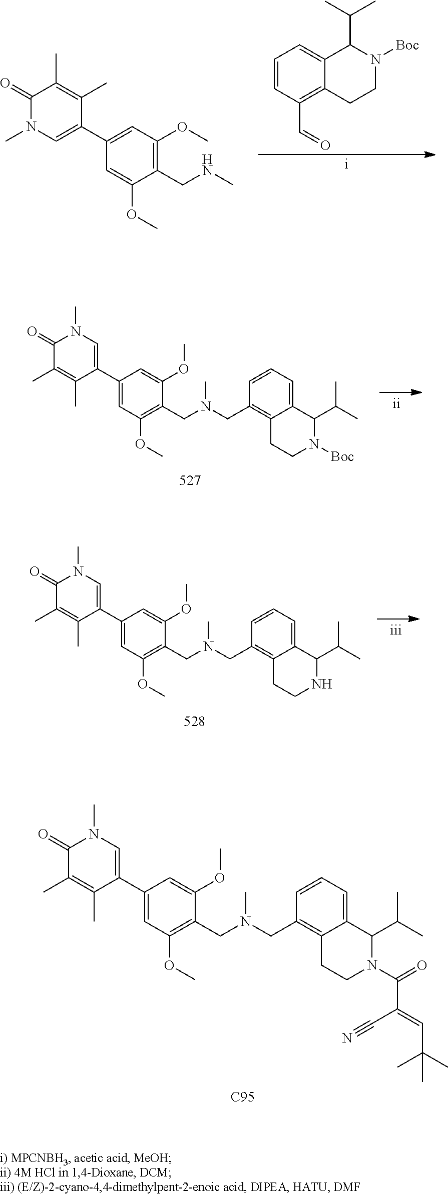

527: Prepared following general procedure 1 using MP-CNBH₃ (1 equiv.) in MeOH. Obtained 230 mg, 80% yield. LCMS m/z=604.4 [M+H]⁺.

528: Prepared following general procedure 2. Obtained 190 mg 98% yield LCMS m/z=504.2 [M+H]⁺. Compound C95: Prepared following general procedure 6. Obtained 44 mg, 31% yield. LCMS m/z=639.4 [M+H].

### Compound C96 and Compound C97

### Enantiomer 1 and Enantiomer 2 of (E/Z)-2-(7-((7-(2,6-dimethoxy-4-(1,4,5-trimethyl-6-oxo-1,6-dihydropyridin-3-yl)benzyl)-4,7-diazaspiro[2.5]octan-4-yl)methyl)-1-isopropyl-1,2,3,4-tetrahydroisoquinoline-2-carbonyl)-5-fluoro 4,4-dimethylpent-2-enenitrile

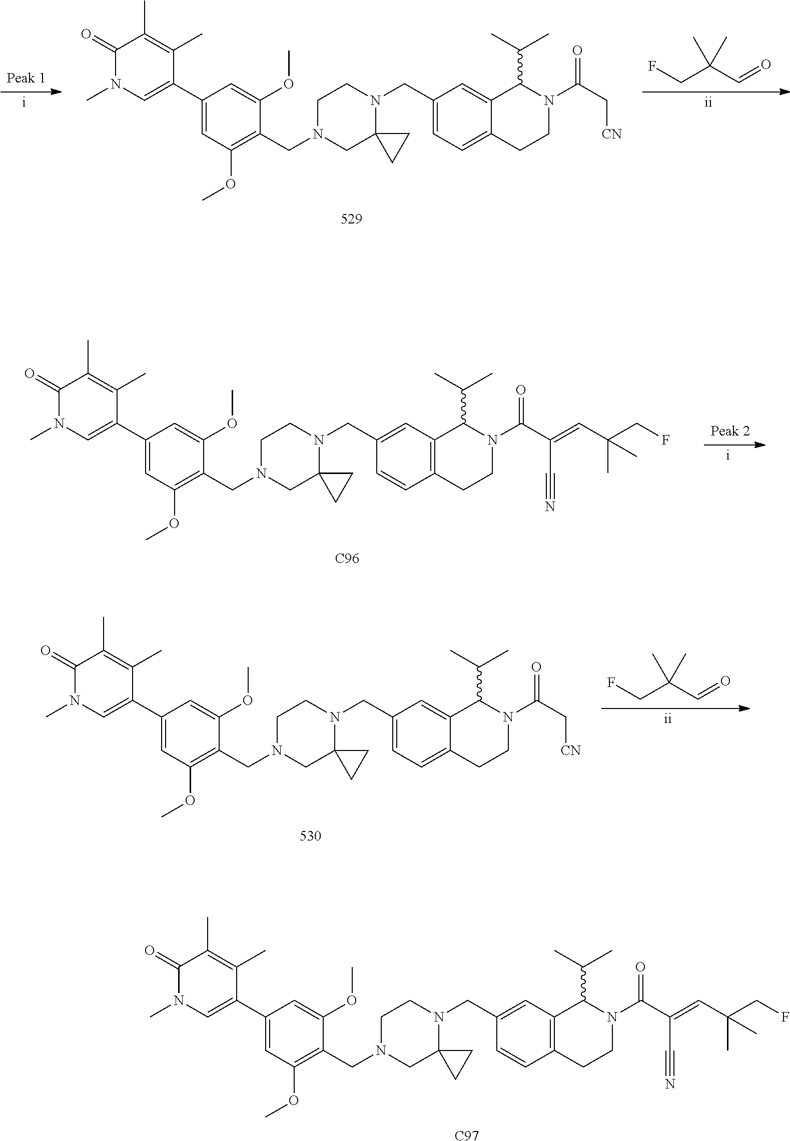

529: Prepared following general 3 using peak 1 from the chiral SFC above. Obtained 200 mg, 86% yield. LCS m/z=652.3 [M+H]⁺.

530: Prepared following general 3 using peak 2 from the chiral SFC above. Obtained 150 mg, 79% yield. LCMS m/z=652.3 [M+H]⁺.

Compound C96: Prepared following general 4. Obtained 38.1 mg, 37.2% yield. LCMS m/z=738.4 [M+H]⁺.

Compound C97: Prepared following general 4. Obtained 34.5 mg, 34.3% yield. LCMS m/z=738.4 [M+H]⁺.

### Compound C98: (E/Z)-2-(5-(((2S,5R)-4-(2,6-dimethoxy-4-(1,4,5-trimethyl-6-oxo-1,6-dihydropyridin-3-yl)benzyl)-2,5-dimethylpiperazin-1-yl)methyl)-1-isopropyl-1,2,3,4-tetrahydroisoquinoline-2-carbonyl)-5-fluoro-4,4-dimethylpent-2-enenitrile

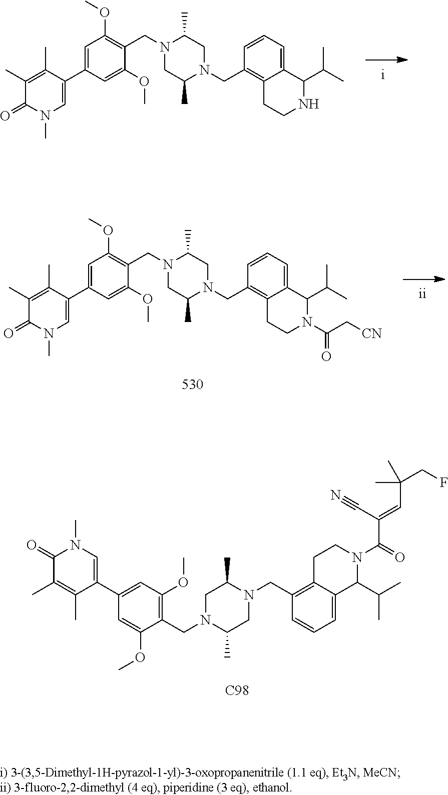

530: By general procedure 3. Obtained 140 mg, 83% yield. LCMS m/z=654.3 [M+H]⁺

Compound C98: By general procedure 4 using 3-fluoro-2,2-dimethylpropanal (4 equiv.) in EtOH. Obtained 20 mg, 16.2% yield, LCMS m/z=740.4 [M+H]⁺.

### Compound C99 (Peak 1): Single Enantiomer of (E/Z)-2-(1-cyclopropyl-5-((7-(2,6-dimethoxy-4-(1,4,5-trimethyl-6-oxo-1,6-dihydropyridin-3-yl)benzyl)-4,7-diazaspiro[2.5]octan-4-yl)methyl)-1,2,3,4-tetrahydroisoquinoline-2-carbonyl)-4,4-dimethylpent-2-enenitrile

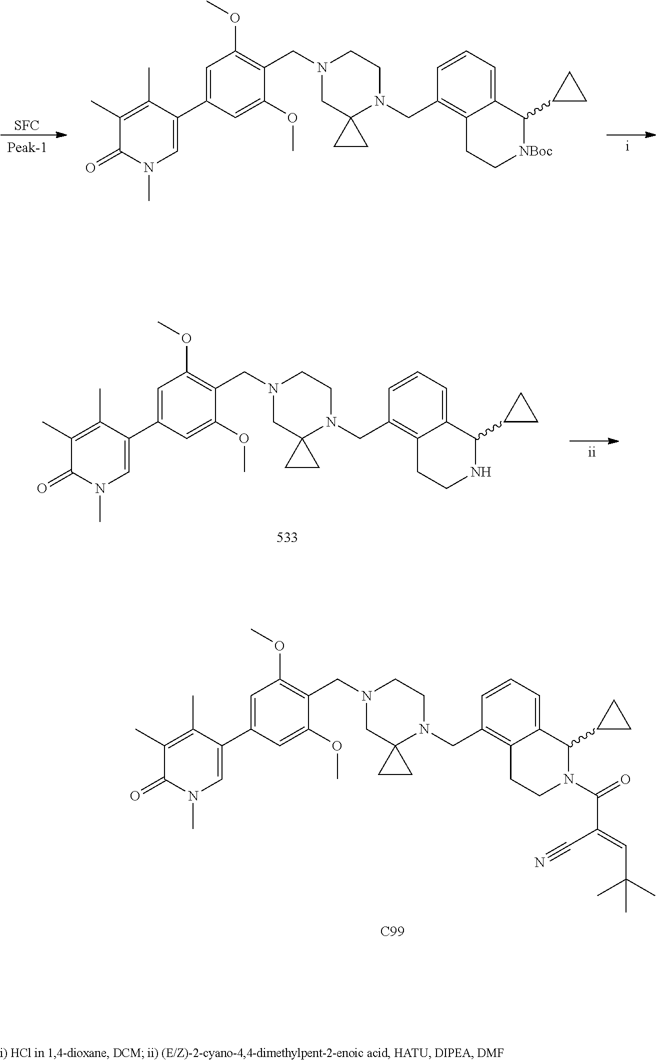 [SMILES: "SFC" ; COc1cc(-c2cn(C)c(=O)c(C)c2C)cc(OC)c1CN1CCN(Cc2cccc3c2CCN(C(=O)OC(C)(C)C)C3C2CC2)C2(CC2)C1 ; COc1cc(-c2cn(C)c(=O)c(C)c2C)cc(OC)c1CN1CCN(Cc2cccc3c2CCNC3C2CC2)C2(CC2)C1 ; COc1cc(-c2cn(C)c(=O)c(C)c2C)cc(OC)c1CN1CCN(Cc2cccc3c2CCN(C(=O)/C(C#N)=C/C(C)(C)C)C3C2CC2)C2(CC2)C1 ; "C99"]

### SFC Purification Method

Racemic tert-butyl 1-cyclopropyl-5-((7-(2,6-dimethoxy-4-(1,4,5-trimethyl-6-oxo-1,6-dihydropyridin-3-yl)benzyl)-4,7-diazaspiro[2.5]octan-4-yl)methy)-3,4-dihydroisoquinoline-2(1H)-carboxylate (130 mg) was purified by asymmetric SFC:

| Column | I Cellulose Z-(250*30) mm, 5 μm | |
|---|---|---|
| Mobile Phase | CO₂: 0.5% isopropylamine in | |
| | MeOH:MeCN [55:45] | |
| Flow rate | 4 | mL/min |
| Back pressure | 100 | bar |
| Wavelength | 254 | nm |
| Cycle time | 8 | min |

Isolated Peak-1: 60 mg. LCMS m/z=683.4 [M+H]⁺

533: By general procedure 2. Obtained 50 mg, 92% yield. LCMS m/z=583.4 [M+H]⁺.

Compound C99: Prepared by following general procedure 6. Obtained 25 mg, 49% yield. LCMS m/z=718.4 [M+H]⁺.

### Compound C100 (Peak 1) & Compound C101 (Peak 2)

### Enantiomer 1 and Enantiomer 2 of (E/Z)-2-(5-((7-(2,6-dimethoxy-4-(1,4,5-trimethyl-6-oxo-1,6-dihydropyridin-3-yl)benzyl)-4,7-diazaspiro[2.5]octan-4-yl)methyl)-1-propyl-1,2,3,4-tetrahydroisoquinoline-2-carbonyl)-4,4-dimethylpent-2-enenitrile

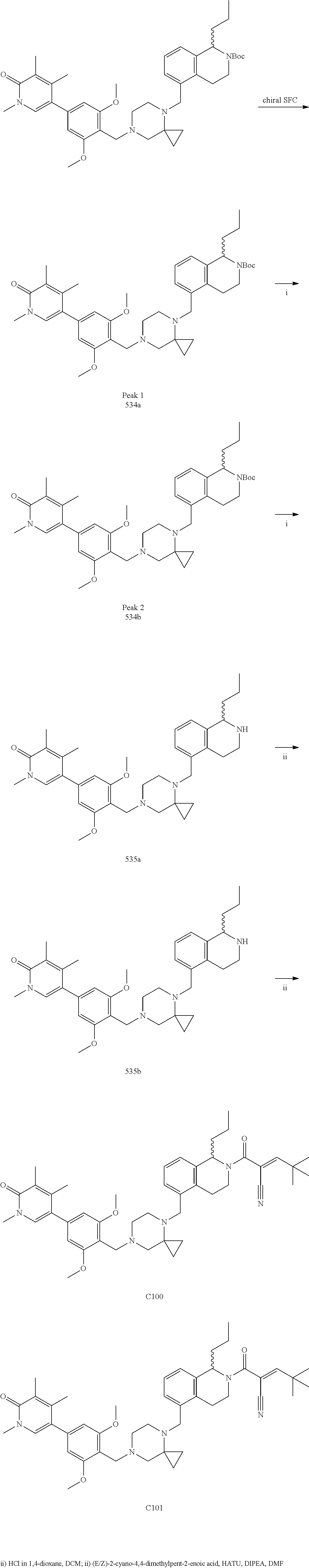

Racemic tert-butyl 5-((7-(2,6-dimethoxy-4-(1,4,5-trimethyl-6-oxo-1,6-dihydropyridin-3-yl)benzyl)-4,7-diazaspiro[2.5]octan-4-yl)methyl)-1-propyl-3,4-dihydroisoquinoline-2(1H)-carboxylate (500 mg) was purified b asymmetric SFC:

| Column | I Cellulose Z-(250*30) mm, 5 μm | |
|---|---|---|
| Mobile Phase | CO₂: 0.5% isopropylamine in | |
| | MeOH:MeCN [55:45] | |
| Flow rate | 4 | mL/min |
| Back pressure | 100 | bar |
| Wavelength | 254 | nm |
| Cycle time | 8 | min |

534a: Isolated Peak 1: 230 mg. LCMS m/z=685.4 [M+H]⁺ and 534b: Peak 2: 236 mg. LCMS m/z=685.3 [M+H]⁺

535a: By general procedure 2 using peak 1 from the SFC above. Obtained 200 mg. LCMS m/z=585.4 [M+H]⁺.

Compound C100: By general procedure 6. Obtained 40 mg, 19.7% yield. LCMS m/z=720.4 [M+H]⁺.

535b: By general procedure 2 using peak 2 from the SFC above. Obtained 195 mg. LCMS m/z=585.4 [M+H]⁺.

Compound C101: By general procedure 6. Obtained 60 mg, 23.7% yield. LCMS m/z=720.4 [M+H]⁺.

### Compound C102 (Peak 1) and Compound C103 (Peak 2)

### Enantiomer 1 and Enantiomer 2 of (E/Z)-2-(5-((4-(2,6-dimethoxy-4-(1,4,5-trimethyl-6-oxo-1,6-dihydropyridin-3-yl)benzyl)-4,7-diazaspiro[2.5]octan-7-yl)methyl)-1-isopropyl-1,2,3,4-tetrahydroisoquinoline-2-carbonyl)-5-fluoro-4,4-dimethylpent-2-enenitrile

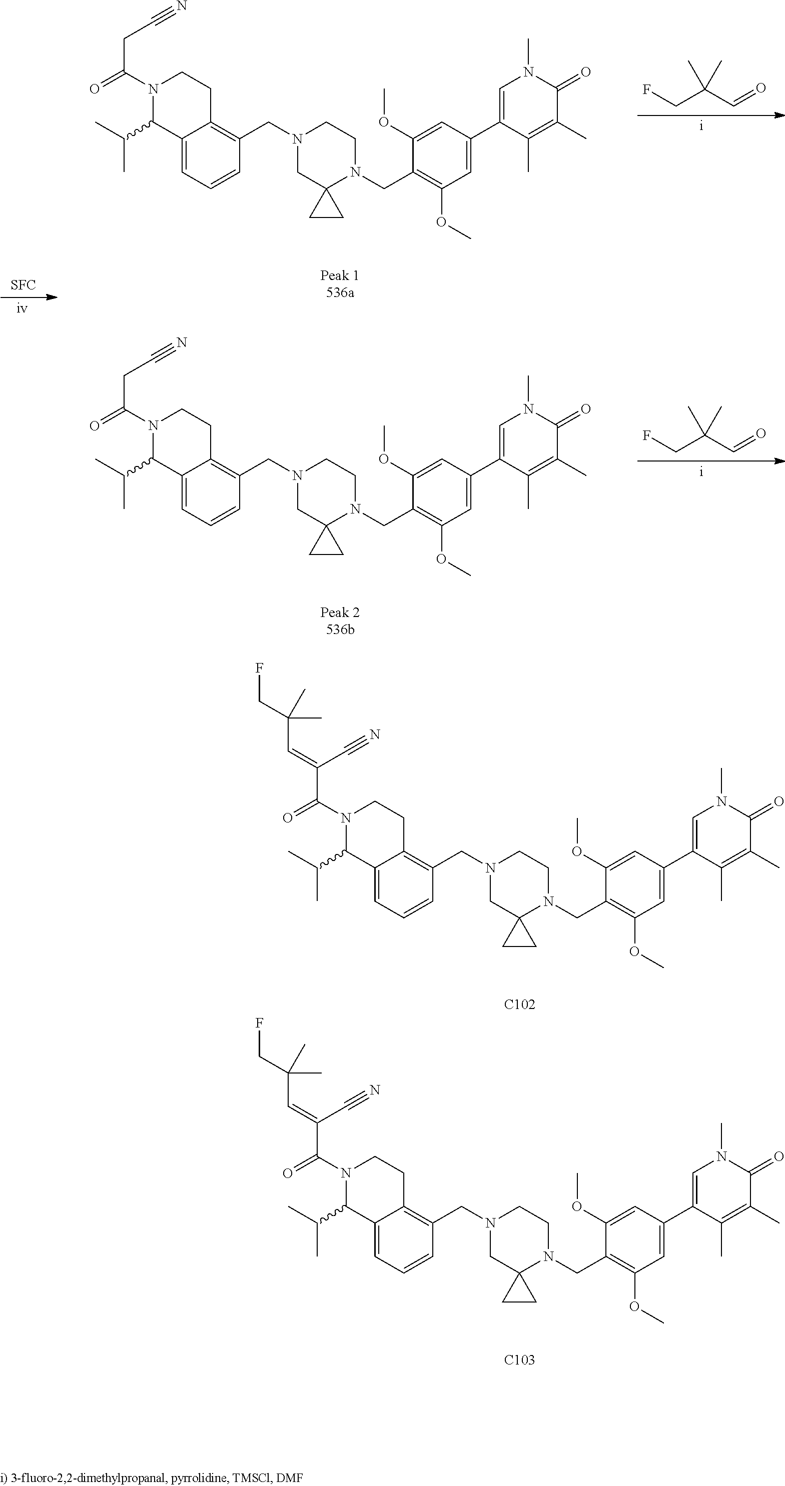

Racemic 3-(5-((4-(2,6-dimethoxy-4-(1,4,5-trimethyl-6-oxo-1,6-dihydropyridin-3-yl)benzyl)-4,7-diazaspiro[2.5]octan-7-yl)methyl)-1-isopropyl-3,4-dihydroisoquinolin-2(1H)-yl)-3-oxopropanenitrile (520 mg) was purified by asymmetric SFC:

| Column | I Cellulose Z-(250*30) mm, 5 μm | |
|---|---|---|
| Mobile Phase | CO₂: 0.5% isopropylamine in | |
| | MeOH:MeCN [55:45] | |
| Flow rate | 4 | mL/min |
| Back pressure | 100 | bar |
| Wavelength | 210 | nm |
| Cycle time | 11 | min |

536a: Isolated Peak 1: 250 mg. LCMS m/z=652.3 [M+H]⁺ and 536b: Peak 2: 240 mg. LCMS m/z=652.3 [M+H]⁺

Compound C102: By general procedure 4e using peak 1 from the SFC above. Obtained 31 mg, 30.7% yield. LCMS m/z=738.4 [M+H]⁺.

Compound C103: By general procedure 4e using peak 2 from the SFC above. Obtained 15 mg, 8.7% yield. LCMS m/z=738.3 [M+H]⁺.

### Compound C104 (Peak 1) and Compound C105 (Peak 2)

### Enantiomer 1 and enantiomer 2 of (E/Z)-2-(5-((4-(2,6-dimethoxy-4-(1,4,5-trimethyl-6-oxo-1,6-dihydropyridin-3-yl)benzyl)-4,7-diazaspiro[2.5]octan-7-yl)methyl)-1-ethyl-1,2,3,4-tetrahydroisoquinoline-2-carbonyl)-6-fluoro-4,4-dimethylpent-2-enenitrile

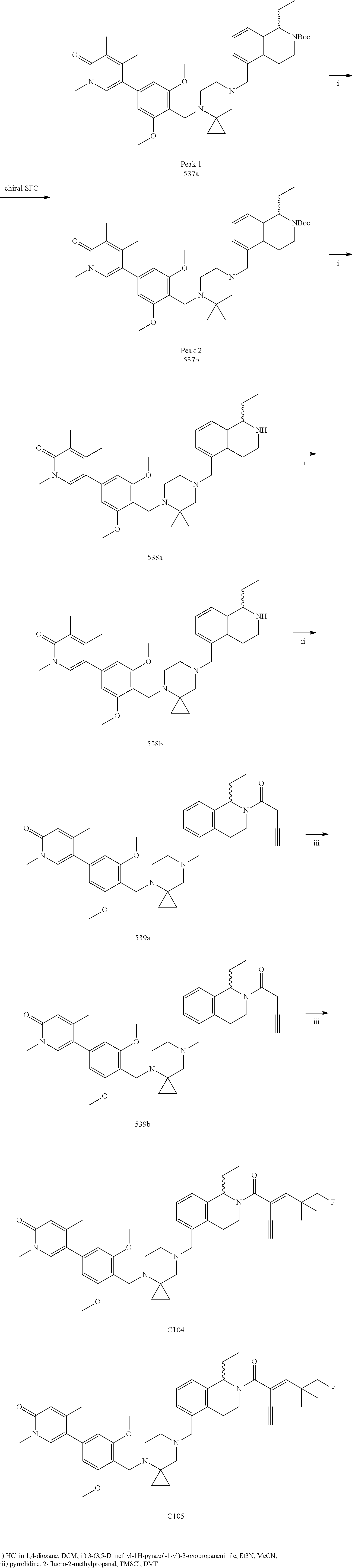

Racemic tert-butyl 5-((4-(2,6-dimethoxy-4-(1,4,5-trimethyl-6-oxo-1,6-dihydropyridin-3-yl)benzyl)-4,7-diazaspiro[2.5]octan-7-yl)methyl)-1-ethyl-3,4-dihydroisoquinoline-2(1H)-carboxylate (900 mg) was purified by assymetric SFC:

| Column | I Cellulose Z-(250*30) mm, 5 μm | |
|---|---|---|
| Mobile Phase | CO₂: 0.5% isopropylamine in | |
| | MeOH:MeCN [55:45] | |
| Flow rate | 4 | mL/min |
| Back pressure | 100 | bar |
| Wavelength | 210 | nm |
| Cycle time | 11 | min |

537a: Peak 1: 450 mg. LCMS m/z=871.4 [M+H]⁺ and 537b: Peak 2: 420 mg. LCMS m/z=671.3 [M+H]⁺

538a: By general procedure 2 using peak 1 from the SFC above. Obtained 440 mg. LCMS m/z=571.3 [M+H]⁺.

538b: By general procedure 3. Obtained 400 ma, 83% yield. LCMS m/z=638.3 [M+H]⁺.

Compound C104: By general procedure 4. Obtained 15 mg, 8.7% yield. LCMS m/z=724.3 [M+H]⁺.

538b: general procedure 2 using peak 2 from the SFC above. Obtained 400 mg. LCMS m/z=571.3[M+H]⁺.

539b: By general procedure 3. Obtained 380 mg, 83% yield. LCMS m/z=638.2 [M+H]⁺.

Compound C105: By general procedure 4. Obtained 20 mg, 11.6% yield. LCMS m/z=724.3 [M+H]⁺.

### Part B—Biological Data

The bifunctional compounds were assayed to Investigate their ability to degrade target proteins in accordance with the following general procedures.

### Assay Protocol 1

BRD9 degradation:

HiBiT-BRD9 KI HEK293 (LgBiT) Cells (Promega CS3023412) were seeded at 8000 cells per well (38 μL) in sterile white bottom 384 well plates (Invitrogen 164610 or Greiner 781080) and incubated overnight at 37° C. with 5% CO₂. The next day, compounds were prepared at 1000× final concentration in DMSO and diluted 1:100 in media (DMEM PAN Biotech P04-03550+10% FBS ATCC 302025). 4 μL compound was added to each well of the cell plate and incubated for 6 h at 37° C. with 5% CO₂ BRD9 expression was quantified using the NanoGlo Lytic Endpoint assay (Promega N3050). The lytic buffer was equilibrated to room temperature for 10-15 min before lytic reagent was prepared by adding substrate (1:50) and LgBit protein (1:100) in lytic buffer. 40 μL lytic reagent mix was added to each well of the cel plate and plates were centrifuged briefly at ˜50g. Plates were incubated with shaking (350 RPM) for 12-24 min before reading using the LUM plus module of a BMG Pherastar FSX. Data was analysed using Dotmatics software and % BRD9 remaining was calculated by normalisation to average data from high and low control wells (DMSO treated cels and no cells respectively).

### BRD7 Degradation:

BRD7 degradation was determined in line with the procedure as outlined above for BRD9, with the exception that HiBiT-BRD7 KI HEK293 (LgBiT) Cells (Promega CS3023410) were seeded at 8000 cells per well (CPW) (36 uL) in sterile white bottom 384 well plates (Invitrogen 164610 or Greiner 781080) and incubated overnight at 37° C. with 5% CO₂.

### BRD4 Degradation:

BRD7 degradation was determined in line with the procedure as outlined above for BRD9, HiBiT-BRD4 HEK293 CRISPR CPM (Promega CS302312) were seeded at 8000 CPW (36 uL) in sterile white bottom 384 well plates (Invitrogen 164610 or Greiner 781080) and incubated overnight at 37° C. with 5% CO₂.

### Assay Protocol 2—Degradation of BRD9 (IF)

A suspension of MV4-11 cells (ATCC CRL-9591) was prepared in phenol red-free assay media (IMDM Thermo Scientific 21056023+10% FBS ATCC 302025) and cells were seeded at 20,000 cells per well (45 μL) in sterile black poly-d-lysine coated 384 well plates (Greiner 781948). Compounds were prepared at 1000× final concentration in DMSO, diluted 1:100 in assay media, and 5 μL compound was added to each well of the cell plate. Cells were incubated for 24 hours at 37° C. with 5% CO₂. All the following incubations for immunofluorescence staining were at room temperature. 15 μL of 16% PFA was added to each well (3.7% final concentration) and the cells were fixed for 15 min then washed twice with DPBS. Cells were permeabilised with 0.1% Triton X-100 for 10 min, Triton X-100 was removed, then blocked with 1% BSA in DPBS for 1 hour. Cells were stained with 25 μL anti-BRD9 E4Q3F antibody (CST 48306) diluted 1:25600 in 1% BSA in DPBS for 2-3 hours. Wels were washed twice with DPBS then incubated with 25 μL of 1% BSA containing a 1:1000 dilution of Anti-rabbit Alexa Fluor™ 647 secondary antibody (Thero Scientific A21244) and 1 μg/mL Hoechst nuclear counter stain (Abcam ab228551) for 1 hour. Wells were washed twice with DPBS prior to imaging on a Perkin Elmer Operetta CLS with 10× air lens. Images were processed using Harmony High-Content Imaging and Analysis Software (Perkin Elmer) and the mean contrast ratio of Alexa Fluor™ 647 in central nuclei was used to quantify BRD9 protein levels. Data was further analysed using Dotmatics software and % BRD9 remaining was calculated by normalisation to average data from high and low control wells (cells treated with DMSO or 100 nM CFT-8634 respectively).

### BRD9 Degradation Results

The degradation of BRD9was detected according to the procedure outlined in Assay Protocol 1 and Assay Protocol 2 for a number of exemplary bifunctional molecules. The results are shown in Table 2 below. In particular, the table shows the degradation efficiency of 1 μM of Indicated example compound at 6 h of treatment.

TABLE 2
data showing degradation efficiency of exemplary compounds:

| | |
|---|---|
| Assay 1 | Assay 2 |

| Compound | DC₅₀ | Dₘₐₓ | DC₅₀ | DCₘₐₓ |
|---|---|---|---|---|
| A2 | +++ | + | +++ | + |
| A3 | ++ | + | ++ | + |
| A4 | +++ | ++ | +++ | ++ |
| A5 | +++ | + | / | / |
| A6 | +++ | +++ | / | / |
| A7 | ++ | + | + | + |
| A8 | + | + | / | / |
| A9 | +++ | + | / | / |
| A10 | +++ | + | +++ | + |
| A11 | ++ | + | / | / |
| A12 | + | + | / | / |
| A13 | ++ | + | / | / |
| A14 | +++ | + | +++ | + |
| A15 | ++ | + | +++ | + |
| A16 | ++ | ++ | +++ | + |
| A17 | ++ | + | / | / |
| A18 | +++ | + | / | / |
| A19 | ++ | + | / | / |
| A20 | ++ | +++ | / | / |
| A21 | +++ | ++ | / | / |
| A22 | ++ | +++ | +++ | +++ |
| A23 | ++ | + | / | / |
| A24 | +++ | + | / | / |
| A25 | +++ | + | / | + |
| A26 | ++ | + | * | + |
| A27 | ++ | + | ++ | + |
| A28 | ++ | + | / | + |
| A29 | ++ | + | / | / |
| A30 | ++ | + | / | NA |
| A31 | ++ | ++ | ++ | + |
| A32 | + | + | +++ | + |
| A33 | +++ | +++ | / | / |
| A34 | ++ | + | / | / |
| A35 | ++ | + | / | / |
| A38 | ++ | + | / | / |
| A39 | ++ | + | / | / |
| A40 | + | ++ | / | / |
| A41 | ++ | ++ | ++ | ++ |
| A42 | + | + | +++ | + |
| A43 | ++ | ++ | +++ | + |
| A44 | ++ | + | / | / |
| A45 | ++ | ++ | / | / |
| A46 | ++ | + | / | / |
| A47 | + | + | / | / |
| A48 | ++ | +++ | +++ | + |
| A49 | ++ | + | +++ | + |
| A50 | +++ | +++ | ++ | +++ |
| A51 | ++ | + | +++ | + |
| A52 | ++ | +++ | / | / |
| A53 | ++ | + | / | + |
| A54 | ++ | + | / | / |
| A55 | + | + | / | / |
| A56 | + | + | / | / |
| A57 | ++ | +++ | / | / |
| A58 | ++ | + | / | / |
| A59 | ++ | ++ | / | / |
| A60 | +++ | + | / | / |
| A61 | +++ | +++ | / | / |
| A62 | ++ | ++ | / | / |
| A63 | ++ | +++ | / | / |
| A64 | ++ | +++ | / | / |
| A65 | ++ | + | / | / |
| A66 | +++ | + | +++ | +++ |
| A67 | ++ | + | / | + |
| A68 | ++ | +++ | +++ | + |
| A69 | ++ | +++ | * | + |
| A70 | + | + | +++ | ++ |
| A71 | ++ | ++ | +++ | +++ |
| A72 | + | ++ | + | + |
| A73 | ++ | +++ | ++ | ++ |
| A74 | * | + | ++ | + |
| A75 | * | + | ++ | +++ |
| A76 | * | + | +++ | ++ |

NA = degradation does not reach 50%, / = not tested.
DC₅₀ key: * = 25 nM to 300 nM, + = >10 nM and <25 nM, ++ = 1.25 to 10 nM, +++ = <1.25 nM.
Dₘₐₓ Key: + = >40% and <70%, ++ = ≥70 and <75%, +++ = ≥75%

The selectivity of a number of exemplary bifunctional compounds to BRD9 degradation relative to degradation of other BRD proteins was detected according to the procedure outlined in assay 1. The results are shown in Table 3 below. In particular, the table shows the degradation efficiency of 1 μM of indicated example compound at 5 h of treatment.

TABLE 3
data comparing degradation of BRD9,
BRD7 and BRD4 by exemplary compounds

| | BRD9 | | BRD7 | | BRD4 | |
|---|---|---|---|---|---|---|
| | DC₅₀ | Dₘₐₓ | DC₅₀ | Dₘₐₓ | DC₅₀ | Dₘₐₓ |
| Compound | (nM) | (%) | (nM) | (%) | (nM) | (%) |
| A4 | +++ | ++ | NA | ## | NA | ## |
| A10 | +++ | + | NA | # | NA | ## |
| A16 | ++ | ++ | NA | # | NA | ## |
| A29 | ++ | + | NA | # | NA | ## |
| A50 | +++ | +++ | NA | # | NA | ## |
| A60 | +++ | + | NA | ## | # | + |
| A61 | +++ | +++ | NA | ## | # | + |
| A68 | ++ | +++ | NA | ## | NA | ## |
| A71 | ++ | ++ | NA | + | NA | # |
| A72 | + | ++ | NA | ## | NA | ## |
| A73 | ++ | +++ | NA | ## | + | +++ |

Key: NA = degradation does not reach 50%, / = not tested.
DC₅₀ key: ## = >1000 nM, # = >25 nM and ≤1000 nM, + = >10 nM and <25 nM, ++ = 1.25 to 10 nM, +++ = <1.25 nM.
Dₘₐₓ key: ## = ≤25%, # = >25% and ≤40%, + = >40% and <70%, ++ = ≥70 and <75%, +++ = ≥75%

### Degradation Results

The degradation of BRD9 was detected according to the procedure outlined in the assay protocols above for a number of exemplary bifunctional molecules. The results are shown in Table 4 below.

TABLE 4

| Ref. | DC₅₀¹ | Dₘₐₓ¹ | DC₅₀² | Dₘₐₓ² |
|---|---|---|---|---|
| BRD9a | + | ++ | | ++ |
| BRD9b | +++ | +++ | +++ | ++ |
| BRD9c | +++ | +++ | +++ | +++ |
| BRD9d | ++ | +++ | + | ++ |
| BRD9e | + | | | + |
| BRD9f | +++ | ++ | +++ | ++ |
| BRD9g | ++ | + | ++ | |
| BRD9h | +++ | +++ | +++ | +++ |
| BRD9i | ++ | ++ | +++ | |
| BRD9j | +++ | | +++ | +++ |
| BRD9k | +++ | | +++ | ++ |
| BRD9l | | ++ | | |
| BRD9m | +++ | +++ | + | |
| BRD9n | | ++ | ++ | |
| BRD9o | | +++ | ++ | |
| BRD9p | | +++ | | +++ |
| BRD9q | | ++ | +++ | |
| BRD9r | | | + | +++ |
| BRD9s | | | +++ | ++ |
| BRD9t | | +++ | + | +++ |
| BRD9u | | + | +++ | + |
| BRD9v | | | +++ | ++ |
| BRD9w | +++ | +++ | +++ | +++ |
| BRD9x | +++ | | +++ | +++ |
| BRD9y | | | +++ | + |
| BRD9z | | | + | +++ |
| BRD9aa | | | | +++ |
| BRD9ab | +++ | | +++ | ++ |
| BRD9ac | ++ | + | | ++ |

Additional Examples:

| | | | | |
|---|---|---|---|---|
| B204 | | | +++ | +++ |
| B205 | | + | ++ | ++ |
| B206 | ++ | +++ | ++ | ++ |
| B207 | ++ | +++ | ++ | +++ |
| B208 | +++ | +++ | +++ | +++ |
| B209 | + | + | ++ | +++ |
| B210 | ++ | ++ | ++ | ++ |
| B211 | + | +++ | + | ++ |
| B212 | ++ | ++ | ++ | ++ |
| B213 | | + | + | ++ |
| B214 | | | + | ++ |

DC₅₀ key: + = ≥200 nM, ++ = ≥25 nM and <200 nM, +++ = <25 nM
Dₘₐₓ key: + = ≥30% and <50%, ++ = ≥50% and <70%, +++ = ≥70%.
¹= determined according to Assay Protocol 1.
²= determined according to Assay Protocol 2.

The selectivity of a number of exemplary bifunctional compounds to BRD9 degradation relative to degradation of other BIRD proteins was detected according to the procedure outlined in assay protocol 1 (unless otherwise stated). The results are shown in Table 5.

TABLE 5
data comparing degradation of BRD9,
BRD7 and BRD4 by exemplary compounds

| | BRD9 | | BRD4 | | BRD7 | |
|---|---|---|---|---|---|---|
| | DC₅₀ | Dₘₐₓ | DC₅₀ | Dₘₐₓ | DC₅₀ | Dₘₐₓ |
| Compound | (nM) | (%) | (nM) | (%) | (nM) | (%) |
| BRD9a | # | + | # | +++ | ## | ## |
| BRD9b | + | ++ | # | +++ | ## | ## |
| BRD9c | +++* | +++* | # | +++ | ## | ## |
| BRD9d | + | ++ | ## | ## | ## | ## |
| BRD9e | ##* | #* | # | +++ | ## | ## |
| BRD9h | +++ | +++ | # | + | ## | # |
| BRD9i | # | + | ## | + | ## | # |
| BRD9o | + | +++ | ## | # | ## | # |
| BRD9ac | #* | +* | ## | # | # | ++ |

Key: * indicates BRD9 degradation measured in accordance with Assay Protocol 2.
DC₅₀ key: ## = >1000 nM, # = >25 nM and ≤1000 nM, + = >10 nM and <25 nM, ++ = 1.25 to 10 nM, +++ = <1.25 nM.
Dₘₐₓ key: ## = ≤25%, # = >25% and ≤40%, + = >40% and <70%, ++ = ≥70 and <75%, +++ = ≥75%;

The degradation of BRD9 was detected according to the procedure outlined in the assay protocols above for a number of exemplary bifunctional molecules. The results are shown in Table 6 below.

TABLE 6

| | |
|---|---|
| Assay protocol 1 | Assay protocol 2 |

| Compound No. | DC₅₀ | Dₘₐₓ | DC₅₀ | Dₘₐₓ |
|---|---|---|---|---|
| B1 | / | + | +++ | + |
| B2 | ++ | +++ | ++ | ++ |
| B3 | + | +++ | + | +++ |
| B4 | ++ | + | ++ | + |
| B5 | ++ | +++ | +++ | +++ |
| B6 | ++ | +++ | +++ | +++ |
| B7 | ++ | + | +++ | + |
| B8 | ++ | + | / | / |
| B9 | +++ | +++ | +++ | +++ |
| B10 | ++ | ++ | / | + |
| B11 | +++ | +++ | +++ | +++ |
| B12 | + | +++ | ++ | +++ |
| B13 | + | +++ | ++ | + |
| B14 | ++ | +++ | ++ | +++ |
| B15 | ++ | +++ | +++ | +++ |
| B16 | +++ | +++ | ++ | +++ |
| B17 | # | + | # | + |
| B18 | ++ | + | / | # |
| B19 | ++ | + | / | + |
| B20 | +++ | +++ | +++ | +++ |
| B21 | ++ | + | +++ | + |
| B22 | ++ | +++ | +++ | + |
| B23 | + | ++ | ++ | +++ |
| B24 | +++ | + | ++ | + |
| B25 | ++ | +++ | ++ | +++ |
| B26 | ++ | +++ | +++ | +++ |
| B27 | / | + | ++ | +++ |
| B28 | / | # | / | + |
| B29 | # | ++ | + | +++ |
| B30 | + | ++ | ++ | +++ |
| B31 | / | + | + | + |
| B32 | # | + | # | + |
| B33 | + | + | / | + |
| B34 | + | + | ++ | + |
| B35 | # | +++ | + | +++ |
| B36 | ++ | + | # | + |
| B37 | + | +++ | +++ | + |
| B38 | + | + | +++ | + |
| B39 | # | +++ | + | + |
| B40 | +++ | + | +++ | + |
| B41 | ++ | +++ | + | +++ |
| B42 | + | + | / | + |
| B43 | +++ | + | ++ | + |
| B44 | +++ | +++ | +++ | + |
| B45 | +++ | +++ | +++ | +++ |
| B46 | + | +++ | ++ | +++ |
| B47 | +++ | +++ | +++ | +++ |
| B48 | / | / | ++ | +++ |
| B49 | +++ | + | + | + |
| B50 | +++ | ++ | + | +++ |
| B51 | ++ | + | / | # |
| B52 | +++ | ++ | # | + |
| B53 | ++ | + | / | + |
| B54 | +++ | +++ | +++ | ++ |
| B55 | + | +++ | # | +++ |
| B56 | +++ | +++ | +++ | +++ |
| B57 | +++ | + | +++ | + |
| B58 | +++ | + | +++ | + |
| B59 | / | + | / | + |
| B60 | / | / | ++ | + |
| B61 | / | / | ++ | +++ |
| B62 | / | / | ++ | +++ |
| B63 | / | / | ++ | + |
| B64 | / | / | +++ | +++ |
| B65 | / | / | ++ | +++ |
| B66 | / | / | +++ | +++ |
| B67 | / | / | ++ | +++ |
| B68 | / | / | +++ | +++ |
| B69 | / | / | ++ | + |
| B70 | / | / | / | + |
| B71 | / | / | ++ | + |
| B72 | / | / | ++ | + |
| B73 | / | / | # | + |
| B74 | / | / | +++ | + |
| B75 | / | / | ++ | + |
| B76 | / | / | ++ | + |
| B77 | / | / | +++ | + |
| B78 | ++ | +++ | + | +++ |
| B79 | / | / | +++ | ++ |
| B80 | / | / | +++ | + |
| B81 | / | / | +++ | +++ |
| B82 | / | / | # | + |
| B83 | / | / | ++ | +++ |
| B84 | / | / | ++ | +++ |
| B86 | / | / | + | + |
| B88 | / | / | + | + |
| B89 | / | / | +++ | +++ |
| B90 | / | / | + | + |
| B91 | / | / | ++ | + |
| B92 | / | / | +++ | + |
| B93 | / | / | ++ | + |
| B94 | ++ | +++ | ++ | + |
| B95 | / | / | +++ | ++ |
| B96 | / | / | ++ | + |
| B98 | / | / | + | +++ |
| B99 | / | / | # | + |
| B100 | / | / | # | ++ |
| B101 | / | / | ++ | + |
| B102 | / | / | # | +++ |
| B103 | / | / | ++ | +++ |
| B104 | / | / | +++ | +++ |
| B106 | / | / | ++ | +++ |
| B107 | / | / | +++ | +++ |
| B108 | / | / | +++ | +++ |
| B109 | / | / | ++ | +++ |
| B110 | / | / | # | + |
| B111 | / | / | + | +++ |
| B112 | / | / | + | +++ |
| B113 | / | / | ++ | +++ |
| B114 | / | / | ++ | +++ |
| B115 | / | / | ++ | +++ |
| B116 | ++ | +++ | +++ | +++ |
| B117 | / | / | ++ | +++ |
| B118 | / | / | +++ | ++ |
| B119 | / | / | + | + |
| B120 | / | / | ++ | ++ |
| B121 | / | / | # | + |
| B122 | ++ | +++ | + | +++ |
| B123 | ++ | +++ | +++ | +++ |
| B124 | +++ | +++ | +++ | +++ |
| B125 | +++ | +++ | +++ | +++ |
| B126 | ++ | +++ | ++ | ++ |
| B127 | ++ | +++ | ++ | +++ |
| B130 | / | / | + | +++ |
| B131 | / | / | ++ | +++ |
| B132 | / | / | + | + |
| B133 | / | / | + | + |
| B134 | / | / | # | + |
| B135 | / | / | ++ | +++ |
| B136 | / | / | ++ | +++ |
| B137 | / | / | ++ | +++ |
| B138 | / | / | +++ | +++ |
| B139 | / | / | + | ++ |
| B140 | / | / | +++ | +++ |
| B141 | / | / | +++ | +++ |
| B142 | / | / | ++ | +++ |
| B143 | / | / | + | ++ |
| B144 | / | / | +++ | +++ |
| B145 | / | / | +++ | +++ |
| B146 | / | / | +++ | +++ |
| B147 | / | / | ++ | +++ |
| B148 | / | / | ++ | + |
| B149 | / | / | +++ | +++ |
| B152 | / | / | ++ | +++ |
| B153 | / | / | +++ | +++ |
| B154 | / | / | + | + |
| B155 | / | / | # | + |
| B156 | / | / | ++ | + |
| B158 | / | / | +++ | +++ |
| B159 | / | / | +++ | + |
| B160 | / | / | ++ | +++ |
| B161 | / | / | +++ | +++ |
| B162 | / | / | +++ | +++ |
| B164 | / | / | +++ | +++ |
| B165 | / | / | +++ | +++ |
| B169 | / | / | ++ | ++ |
| B173 | / | / | +++ | +++ |
| B174 | / | / | +++ | +++ |
| B175 | / | / | # | + |
| B180 | / | + | / | + |
| B181 | / | + | + | + |
| B182 | # | + | ++ | + |
| B183 | # | + | ++ | +++ |
| B184 | / | + | ++ | + |
| B185 | + | + | ++ | +++ |
| B186 | ++ | +++ | +++ | +++ |
| B187 | ++ | +++ | ++ | +++ |
| B188 | / | / | +++ | +++ |
| B189 | / | / | ++ | +++ |
| B190 | / | / | # | + |
| B191 | / | / | +++ | +++ |
| B192 | / | / | ++ | +++ |
| B193 | / | / | +++ | +++ |
| B194 | / | / | ++ | + |
| B195 | / | / | +++ | +++ |
| B196 | / | / | ++ | ++ |
| B197 | / | / | # | + |
| B198 | / | / | ++ | +++ |
| B199 | / | / | # | + |
| B200 | / | / | ++ | +++ |
| B201 | / | / | # | + |
| B202 | / | / | +++ | +++ |
| B203 | / | / | ++ | +++ |
| B215 | / | / | # | + |
| C1 | / | / | +++ | +++ |
| C2 | / | / | ++ | +++ |
| C3 | / | / | # | + |
| C4 | / | / | ++ | +++ |
| C5 | / | / | + | +++ |
| C6 | / | / | +++ | +++ |
| C7 | / | / | +++ | +++ |
| C8 | / | / | +++ | +++ |
| C9 | # | ++ | # | + |
| C10 | / | / | ++ | +++ |
| C11 | / | / | ++ | + |
| C12 | / | / | # | ++ |
| C13 | / | / | +++ | +++ |
| C14 | / | / | # | +++ |
| C15 | / | / | +++ | +++ |
| C16 | / | / | +++ | +++ |
| C17 | / | / | ++ | +++ |
| C18 | / | / | ++ | +++ |
| C19 | / | / | ++ | +++ |
| C20 | / | / | +++ | +++ |
| C21 | / | / | ++ | ++ |
| C22 | / | / | ++ | +++ |
| C23 | / | / | +++ | +++ |
| C24 | / | / | +++ | +++ |
| C25 | / | / | ++ | +++ |
| C26 | / | / | + | +++ |
| C27 | ## | + | ## | + |
| C28 | / | # | # | + |
| C29 | / | / | ++ | ++ |
| C30 | / | / | # | + |
| C31 | / | / | ++ | +++ |
| C32 | / | / | # | ++ |
| C33 | / | / | ++ | +++ |
| C34 | / | / | # | + |
| C35 | / | / | +++ | +++ |
| C36 | / | / | + | + |
| C37 | / | / | +++ | +++ |
| C38 | / | / | ++ | ++ |
| C39 | / | / | +++ | +++ |
| C40 | / | / | ++ | ++ |
| C41 | / | / | ++ | +++ |
| C42 | / | / | ++ | + |
| C43 | / | / | # | +++ |
| C44 | / | / | ++ | ++ |
| C45 | / | / | + | + |
| C46 | / | / | ++ | + |
| C47 | / | / | ++ | +++ |
| C48 | / | / | ++ | +++ |
| C49 | / | / | / | + |
| C50 | / | / | # | ++ |
| C51 | / | / | # | + |
| C52 | / | / | + | + |
| C53 | / | / | # | + |
| C54 | / | / | # | + |
| C55 | / | / | # | +++ |
| C56 | / | / | # | +++ |
| C57 | / | / | # | + |
| C58 | / | / | + | +++ |
| C59 | / | / | ++ | + |
| C60 | / | / | ++ | ++ |
| C61 | / | / | +++ | +++ |
| C62 | / | / | + | +++ |
| C63 | / | / | ++ | +++ |
| C64 | / | / | # | + |
| C65 | / | / | ++ | + |
| C66 | / | / | ++ | + |
| C67 | / | / | / | + |
| C68 | / | / | # | + |
| C69 | / | / | + | + |
| C70 | / | / | # | + |
| C71 | / | / | # | + |
| C72 | / | / | # | +++ |
| C73 | / | / | ++ | +++ |
| C74 | / | / | # | +++ |
| C75 | / | / | # | +++ |
| C76 | / | / | ++ | ++ |
| C77 | / | / | +++ | +++ |
| C78 | / | / | +++ | +++ |
| C79 | / | / | # | + |
| C80 | / | / | ++ | ++ |
| C81 | / | / | ++ | +++ |
| C82 | / | / | # | + |
| C83 | / | / | # | + |
| C84 | / | / | ++ | +++ |
| C85 | / | / | # | +++ |
| C86 | / | / | # | +++ |
| C87 | / | / | + | ++ |
| C88 | / | / | # | + |
| C89 | / | / | + | + |
| C90 | / | / | ## | + |
| C91 | / | / | +++ | +++ |
| C92 | / | / | / | + |
| C93 | / | / | +++ | +++ |
| C94 | / | / | +++ | +++ |
| C95 | / | / | + | + |
| C96 | / | / | ++ | +++ |
| C97 | / | / | + | ++ |
| C98 | / | / | + | +++ |
| C99 | / | / | +++ | +++ |
| C100 | / | / | +++ | +++ |
| C101 | / | / | # | + |
| C102 | / | / | # | + |
| C103 | / | / | +++ | +++ |
| C104 | / | / | # | + |
| C105 | / | / | +++ | +++ |
| C106 | / | / | +++ | +++ |
| C107 | / | / | + | + |

Key: NA = degradation does not reach 50%, / = not tested.
DC₅₀ key: ## = >1000 nM, # = >25 nM and ≤1000 nM, + = >10 nM and <25 nM, ++ = 1.25 to 10 nM, +++ = <1.25 nM.
Dₘₐₓ key: ## = ≤25%, # = >25% and ≤40%, + = >40% and <70%, ++ = ≥70 and <75%, +++ = ≥75%

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A bifunctional molecule comprising the general formula:
TBL-L-Z
wherein TBL is a target protein binding ligand that binds BRD9;
L is a linker; and
Z comprises a structure according to formula (I): 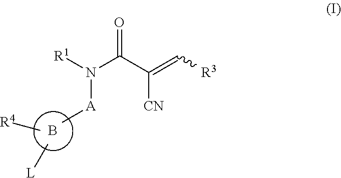
wherein
R¹ is selected from C₁ to C₆ alkyl, benzyl, substituted benzyl, carbocyclyl, substituted carbocyclyl, heterocyclyl and substituted heterocyclyl, optionally wherein the C₁ to C₆ alkyl is substituted with one or more heteroatoms selected from halo, N, O and S and/or is substituted with a carbocyclic or heterocyclic group;
A is absent or is CR²R^{2′};
B is selected from aryl, heteroaryl, substituted aryl and substituted heteroaryl;
R² and R^{2′} are each independently selected from H and C₁ to C₆ alkyl, optionally wherein the C₁ to C₆ alkyl is substituted with one or more heteroatoms selected from halo, N, O or S, or wherein R² and R^{2′} together form a 3-, 4-, 5- or 6-membered carbocyclic or heterocyclic ring;
R³ is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S;
R⁴ is H, C₁ to C₆ alkyl, optionally wherein the C₁ to C₆ alkyl is substituted with one or more heteroatoms selected from N, O or S;
or wherein R¹ and R⁴ together form a 5-, 6-, or 7-membered heterocyclic ring;
or wherein when A is CR²R^{2′}:
R¹ and R² together form a 5-, 6-, or 7-membered heterocyclic ring; or
R² and R⁴ together form a 5-, 6-, or 7-membered heterocyclic or carbocyclic ring;
wherein L shows the point of attachment of the linker;
and
wherein the BRD9 binder (TBL) is of formula 1a: 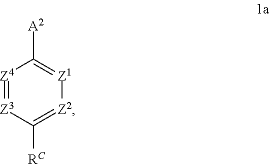
wherein:
Z¹ is N or CR^{A};
Z² is N or CR^{B};
Z³ is N or CR^{D};
Z⁴ is N or CR^{E};
wherein no more than 3 of Z¹, Z², Z³ and Z⁴ are N;
R^{A} and R^{E} are each independently selected from the group consisting of —H, —O—C₁₋₃alkyl and —C₁₋₃alkyl;
R^{B} and R^{D} are each independently selected from the group consisting of —O—C₁₋₃alkyl, —H, —OH, halogen, —NH₂, —C₁₋₃alkyl, —O—C₁₋₃haloalkyl, —C₁₋₃alkyl-O—C₁₋₃alkyl, 4-7 membered heterocycloalkyl, —C₁₋₃alkyl-SO₂—C₁₋₃alkyl, —C₁₋₃alkyl-NH₂, —C₁₋₃alkyl-N(—C₁₋₃alkyl)₂, —N(C₁₋₃ alkyl)₂, —NH—R^{F};
R^{F} is selected from —SO₂—C₁₋₃alkyl and —C₁₋₃alkyl, wherein the —C₁₋₃alkyl is optionally substituted with a 5 to 6 membered heteroaryl;
alternatively, R^{A} and R^{B} taken together form a benzene ring; alternatively, R^{C} and Z² or R^{C} and Z³ taken together form a 5-7 membered heterocycloalkyl optionally substituted with —C₁₋₃alkyl;
R^{C} is selected from the group consisting of —H, —Y—R^{G}, —NH₂, —C₁₋₃alkyl and 4-7 membered heterocycloalkyl;
Y is absent or is selected from the group consisting of —CR^{H}R^{I}—, —SO₂— and —CO—;
R^{H} and R^{I} are each independently selected from —H or —C₁₋₃alkyl; or R^{H} and R^{I} taken together form a —C₃₋₄cycloalkyl,
R^{G} is selected from the group consisting of —NH₂, —OH, —C₁₋₃alkyl, —N(R^{J}R^{K}), —O—R^{L}, aryl, 5-6 membered heteroaryl, wherein the aryl and heteroaryl are optionally and independently substituted with one or more halogen, optionally substituted 4- to 7-membered monocyclic heterocycloalkyl, and optionally substituted 7- to 12-membered bicyclic heterocycloalkyl, which monocyclic or bicyclic heterocycloalkyl are optionally substituted with one or more groups independently selected from halogen, —OH, —NH₂, —C₁₋₃alkyl, —NHC₁₋₃alkyl, —N(C₁₋₃alkyl)₂, —O—C₁₋₃alkyl and —CH₂—R^{M1};
R^{M1} is selected from 5-10 membered mono- or bicyclic aryl or heteroaryl, which is optionally substituted with —NH₂, —OH, halogen, —CN, C₁₋₃alkyl, —O—C₁₋₃alkyl;
R^{J} is —H or —C₁₋₃alkyl;
R^{K} is selected from the group consisting of —C₁₋₃alkyl, —C₂₋₃alkyl-N(C₁₋₃alkyl)₂, —C₂₋₃alkyl-NHC₁₋₃alkyl, optionally substituted 4- to 7-membered monocyclic heterocycloalkyl, and optionally substituted 7- to 12-membered bicyclic heterocycloalkyl, which monocyclic or bicyclic heterocycloalkyl is optionally substituted with —C₁₋₃alkyl;
R^{L} is —C₁₋₃alkyl or a 4-7 membered heterocycloalkyl, which heterocycloalkyl is optionally substituted with C₁₋₃alkyl;
wherein when R^{C} is Y—R^{G}, R^{B} and R^{D} are each independently selected from —H, —OH, halogen, —NH₂, —CN, —C₁₋₃alkyl, —C₁₋₃haloalkyl, —O—C₁₋₃alkyl, —O—C₁₋₃haloalkyl and —C₁₋₃alkyl-O—C₁₋₃alkyl; wherein at least one of the substituents R^{A} to R^{E} is not hydrogen;
and
A² is selected from formulae 1b or 1c: 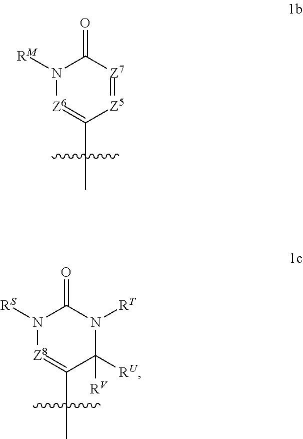
wherein the wavy fines intersect the bond between A² and the carbon atom positioned ortho to R^{A} and R^{E};
R^{M} is selected from the group consisting of optionally substituted C₁₋₆alkyl, optionally substituted C₂₋₆alkenyl, optionally substituted C₁₋₆heteroalkyl, optionally substituted C₃₋₁₀carbocyclyl, C₂₋₆alkynyl and H;
Z⁵ is N or CR^{O};
Z is N or CR^{P};
Z⁷ is N or CR^{N};
wherein only one of Z⁵, Z⁶ and Z⁷ is N;
Z⁸ is CR^{W} or N;
R^{N} is selected from the group consisting of halogen, optionally substituted —C₁₋₆alkyl, —H, C(O)C₁₋₅alkyl, —NH₂, optionally substituted amino, —OH, cyano, optionally substituted C₁₋₆heteroalkyl, optionally substituted C₃₋₁₀ carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl and thiol;
R^{O} is selected from the group consisting of H, halogen, cyano, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl, hydroxy, thiol and optionally substituted amino;
R^{P} is selected from the group consisting of H, halogen, optionally substituted C₁₋₆alkyl, optionally substituted C₁₋₆heteroalkyl, optionally substituted C₃₋₁₀carbocyclyl and optionally substituted C₆₋₁₀aryl;
alternatively, R^{N} and Z⁵ taken together, combine to form an optionally substituted C₆₋₁₀arene or optionally substituted C₂₋₉heteroarene; optionally wherein R^{N} and R^{O} taken together with the carbon atoms to which they are joined, combine to form an optionally substituted C₆₋₁₀arene or optionally substituted C₂₋₉heteroarene;
R^{S} is selected from the group consisting of H, optionally substituted C₁₋₆alkyl, optionally substituted C₁₋₆heteroalkyl and optionally substituted C₃₋₁₀carbocyclyl;
R^{T} is selected from the group consisting of H, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl, optionally substituted sulfone and optionally substituted sulfonamide, or R^{T} and R^{U} together with the atoms to which each is attached, form an optionally substituted C₂₋₉heterocyclyl;
R^{U} and R^{V} are each independently selected from the group consisting of H, halogen, hydroxyl, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl, thiol, optionally substituted sulfone and optionally substituted amino;
alternatively, R^{T} and R^{U} together with the atoms to which each is attached, form an optionally substituted C₂₋₉heterocyclyl;
R^{W} is selected from the group consisting of H, halogen, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl and optionally substituted C₂₋₉heteroaryl;
and
wherein the BRD9 binder is attached to the linker at any suitable position; and
(iii) wherein the bifunctional molecule is not:

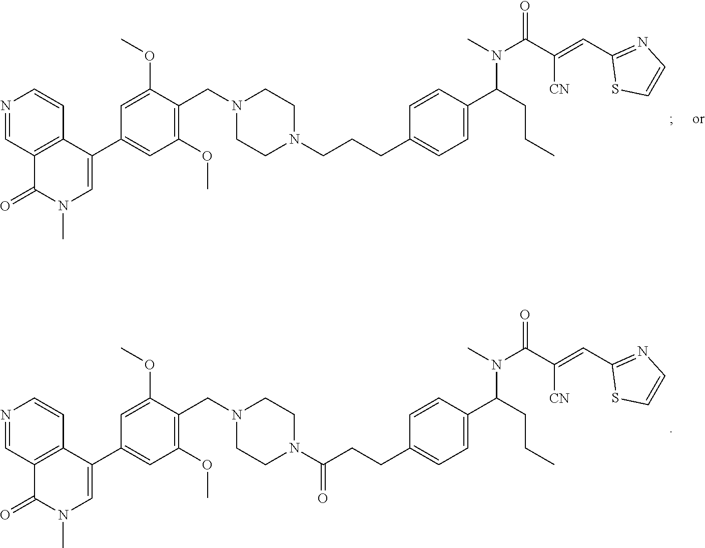 [SMILES: CCCC(c1ccc(CCCN2CCN(Cc3c(OC)cc(-c4cn(C)c(=O)c5cnccc45)cc3OC)CC2)cc1)N(C)C(=O)/C(C#N)=C/c1nccs1 ; CCCC(c1ccc(CCC(=O)N2CCN(Cc3c(OC)cc(-c4cn(C)c(=O)c5cnccc45)cc3OC)CC2)cc1)N(C)C(=O)/C(C#N)=C/c1nccs1]

2. The bifunctional molecule of claim 1, wherein up to 1 of Z¹, Z², Z³ and Z⁴ is N.

3. The bifunctional molecule of claim 1 or claim 2, wherein the BRD9 binder is of formula 1a′: 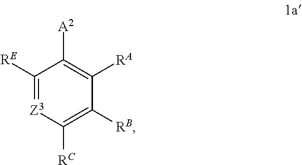
wherein:
R^{A}, R^{B}, R^{C}, R^{E}, Z³ and A² are as defined in claim 1 or 2.

4. The bifunctional molecule of any one of claims 1 to 3, wherein A² is selected from formula 1b′, wherein formula 1b′ is: 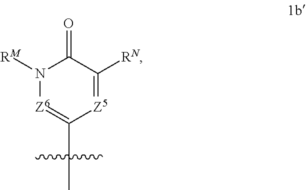
wherein the wavy line intersects the bond between A² and the carbon atom positioned ortho to R^{A} and R^{E};
R^{M} is selected from the group consisting of —C₁₋₅alkyl, -cyclopropyl, —C₁₋₄haloalkyl and H;
R^{N} is selected from the group consisting of halogen, —C₁₋₅alkyl, —C₁₋₃haloalkyl, —H, C(O)C₁₋₅alkyl, —NH₂, —NHC₁₋₃alkyl and —OH;
Z⁵ is N or CR^{O}
Z⁶ is N or CR^{P} wherein only one of Z⁵ and Z⁶ may be N;
R^{O} is H or —C₁₋₃alkyl;
R^{P} is H or —C₁₋₃alkyl;
wherein only one of R^{O} and R^{P} may be —C₁₋₃alkyl;
alternatively, R^{N} and Z⁵ taken together form a benzene ring or a 5-6 membered heteroarene ring, each of which rings can be optionally and independently substituted with one or more groups selected from halogen, —OH, —NH₂, —NH—C₁₋₃alkyl and —C₁₋₅alkyl, C₁₋₅haloalkyl, C₁₋₅alkoxy, C₁₋₄haloalkoxy, 1d, C₃₋₅azacycloalkyl, C₂₋₅alkenyl, C₂₋₅alkynyl, C₃₋₅cycloalkyl, wherein the —C₁₋₅alkyl group can be optionally substituted with 5-6 membered heteroaryl or phenyl; 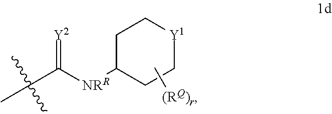
wherein
Y² is NR^{R} or O;
Y¹ is S(O)ₐ or NR^{R};
each R^{R} is independently H or C₁₋₄alkyl;
each R^{Q} is independently selected from the group consisting of C₁₋₄alkyl, C₁₋₄haloalkyl, halogen and —C(O)C₁₋₃alkyl;
a is 0 to 2; and
r is 0 to 3.

5. The bifunctional molecule of any one of claims 1 to 4, wherein the BRD9 binder is of formula 1e, 1f or 1g: 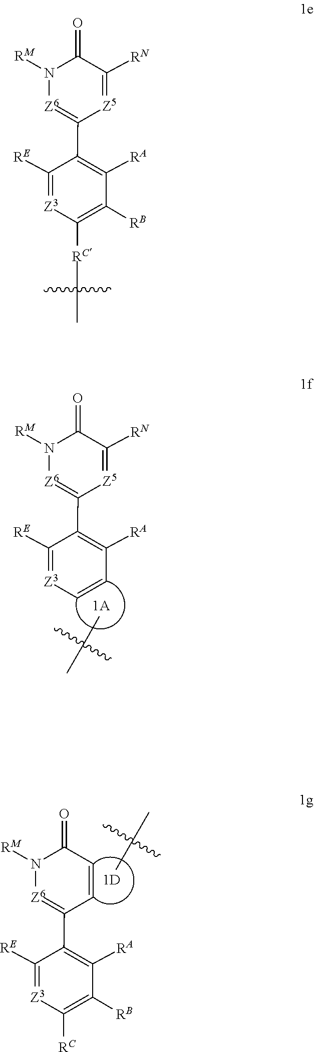
wherein the wavy line intersects the bond between the BRD9 binder and the linker;
wherein R^{A}, R^{B}, R^{C}, R^{E}, R^{M}, R^{N}, Z³, Z⁵ and Z are as defined in any one of claims 1 to 4;
wherein R^{C′} is absent, or is as defined for R^{C} in any one of claims 1 to 4;
ring 1A is a 5-7 membered heterocycloalkane optionally substituted with —C₁₋₃alkyl; and
ring 1D is an optionally substituted C₆₋₁₀aryl or optionally substituted C₂₋₉heteroaryl.

6. The bifunctional molecule of claim 5, wherein ring 1A:
(i) comprises one or two heteroatoms independently selected from the list consisting of N, S and O; or
(ii) is selected from the list consisting of pyrrolidine, piperidine, piperazine, morpholine, oxolane, oxane, tetrahydrothiophene and thiane.

7. The bifunctional molecule of any one of claims 1 to 5, wherein the BRD9 binder is of formula 1e, 1f′ or 1g′: 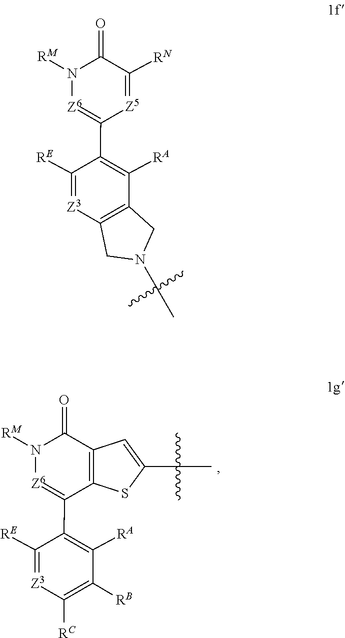
wherein the wavy line intersects the bond between the BRD9 binder and the linker; and
wherein R^{A}, R^{B}, R^{C}, R^{E}, R^{M}, R^{N}, Z³, Z⁵ and Z⁶ are as defined in any one of claims 1 to 4.

8. The bifunctional molecule of any one of claims 1 to 7, wherein R^{A}, R^{B}, R^{C}, R^{D} and R^{E}are independently selected from —O—C₁₋₃alkyl, —H, halogen, —O—C₁₋₃haloalkyl, —OH, —NH₂, —C₁₋₃alkyl, —C₁₋₃alkyl-NH₂, —C₁₋₃alkyl-N(—C₁₋₃alkyl)₂ and —N(C₁₋₃alkyl)₂.

9. The bifunctional molecule of any one of claims 1 to 8, wherein:
(i) at least two of R^{A}, R^{B}, R^{D} and R^{E} are —H; and/or
(ii) at least one of R^{A}, R^{B}, R^{D} and R^{E} is selected from the group consisting of —O—C₁₋₃alkyl, —H, halogen and —O—C₁₋₃haloalkyl.

10. The bifunctional molecule of any one of claims 1 to 9, wherein R^{M} is —C₁₋₅alkyl.

11. The bifunctional molecule of any one of claims 1 to 10, wherein R^{N} is —C₁₋₅alkyl or halogen, or R^{N} and Z⁵ taken together form an optionally substituted 5-6 membered heteroarene or benzene ring, optionally wherein:
(i) the optionally substituted 5-6 membered heteroarene ring comprises one or more heteroatoms selected from the group consisting of N, S and O;
(ii) the optionally substituted 5-6 membered heteroarene ring is an N- or S-heteroarene; or
(iii) the optionally substituted 5-6 membered heteroarene ring is any one selected from the optionally substituted group consisting of pyridine, pyrrole, imidazole, pyrimidine, thiophene and pyrazole.

12. The bifunctional molecule of any one of claims 1 to 11, wherein the BRD9 binder is any one of formulae 1ea to 1eh and 1fa to 1ﬁ and 1ga: 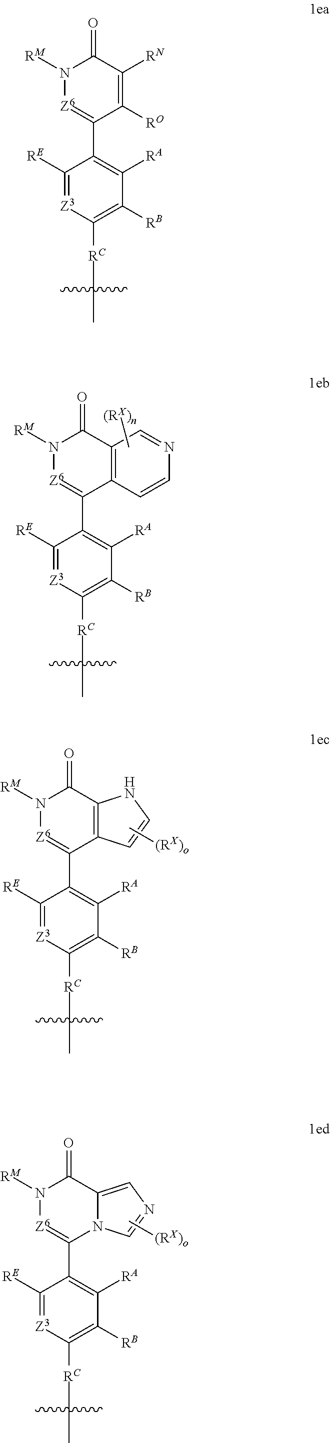 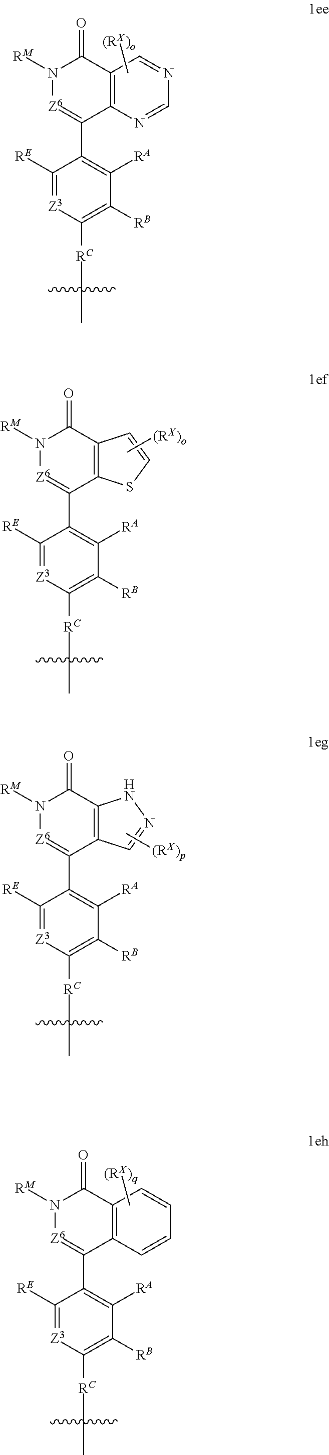 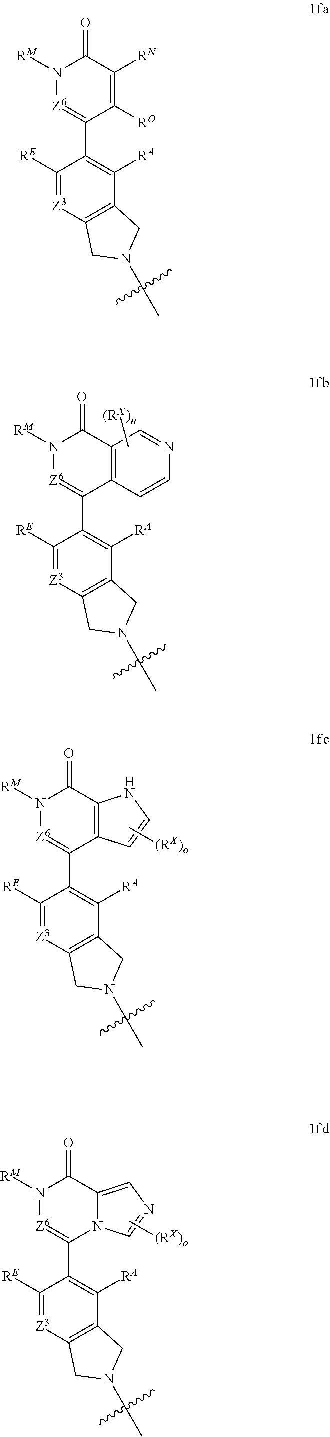 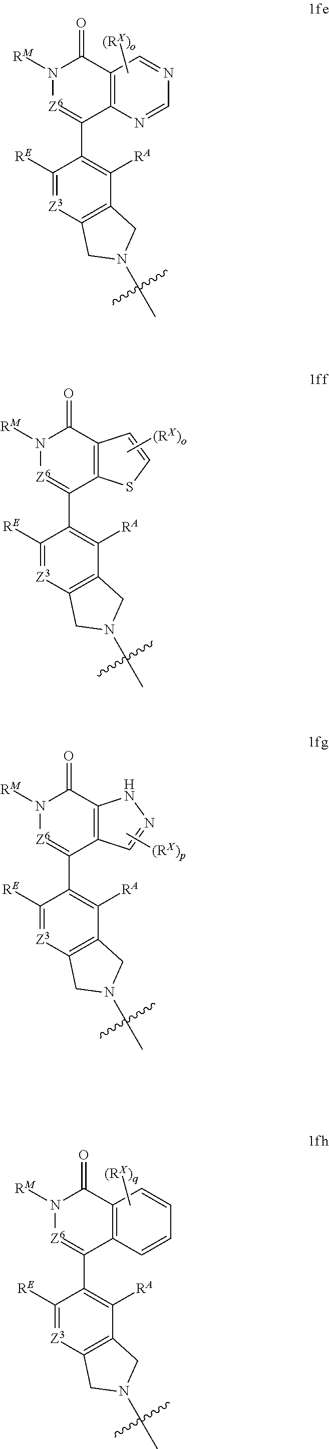 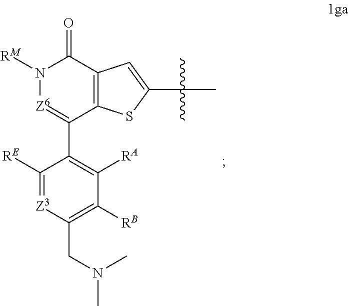
wherein the wavy line intersects the bond between the BRD9 binder and the linker;
R^{A}, R^{B}, R^{E}, R^{M}, Z³ and Z⁸ are as defined in any one of claims 1 to 11;
R^{C} is absent, or is as defined in any one of claims 1 to 11;
R^{N} is selected from the group consisting of halogen, —C₁₋₅alkyl, —C₁₋₃haloalkyl, —H, C(O)C₁₋₅alkyl, —NH₂, —NHC₁₋₃alkyl and —OH;
R^{O} is H or —C₁₋₃alkyl;
each R^{X} is independently selected from the group consisting of halogen, —OH, —NH₂, —NH—C₁₋₃alkyl —C₁₋₅alkyl, C₁₋₅haloalkyl, C₁₋₅alkoxy and C₁₋₄haloalkoxy;
n is 0 to 3;
o is 0 to 2;
p is 0 or 1; and
q is 0 to 4.

13. The bifunctional molecule of any one of claims 1 to 12, wherein the BRD9 binder is according to formula 1ea′: 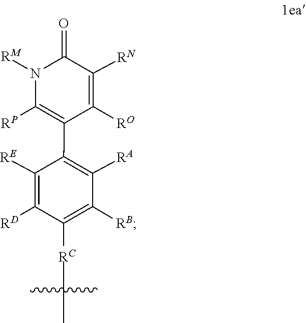
wherein the wavy line intersects the bond between the BRD9 binder and the linker;
R^{A} and R^{E} are each independently selected from H and —O—C₁₋₃alkyl;
R^{B} and R^{D} are each independently selected from —O—C₁₋₃alkyl, —H, -halo, —C₁₋₃alkyl, and —O—C₁₋₃haloalkyl;
R^{C} is absent, or is —Y—R^{G};
Y is selected from the group consisting of —CR^{H}R^{I}—, and —CO—;
R^{H} and R^{I} are each independently selected from —H or —C₁₋₃alkyl; or R^{H} and R^{I} taken together form a —C₃₋₄cycloalkyl;
R^{G} is selected from the group consisting of —N(R^{J}R^{K})—, —N(C₁₋₃alkyl)(optionally substituted 4- to 7-membered monocyclic heterocycloalkylene), or —N(C₁₋₃alkyl)(optionally substituted 7- to 12-membered bicyclic heterocycloalkylene)); —O—;
optionally substituted 4- to 7-membered monocyclic heterocycloalkylene; and optionally substituted 7- to 12-membered heterocycloalkylene;
R^{J} and R^{K}are as defined in claim 1;
R^{M} is C₁₋₃alkyl; and
R^{N}, R^{O} and R^{P} are each independently selected from the group consisting of halo, —C₁₋₃alkyl, and —C₁₋₃haloalkyl.

14. The bifunctional molecule of any one of claims 1 to 12, wherein the BRD9 binder is an one of formulae 1h to 1z and 2a to 2g: 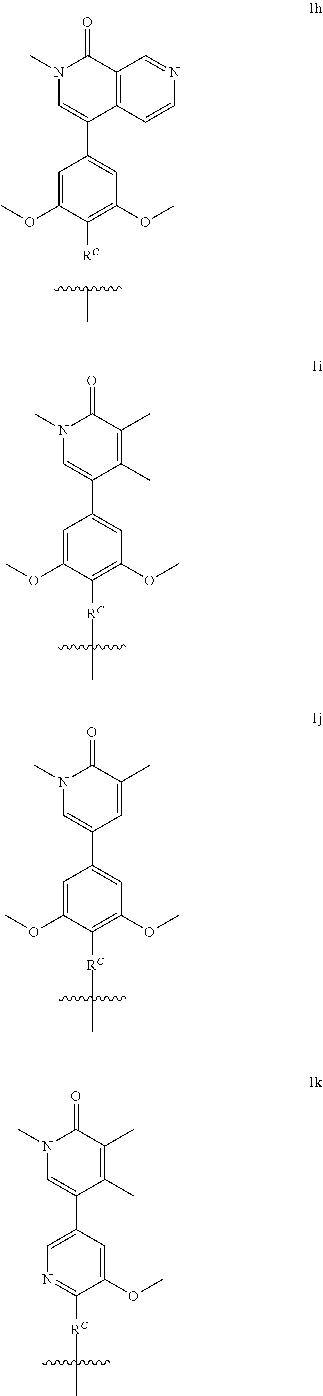 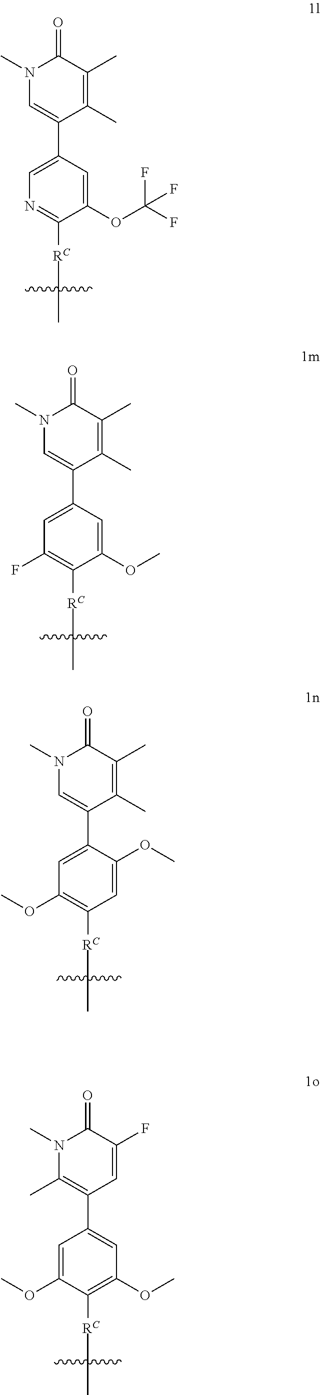 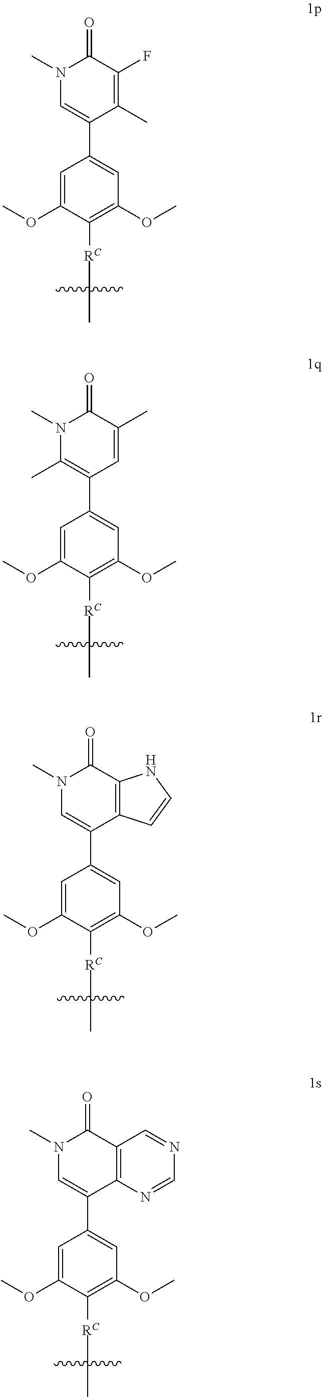 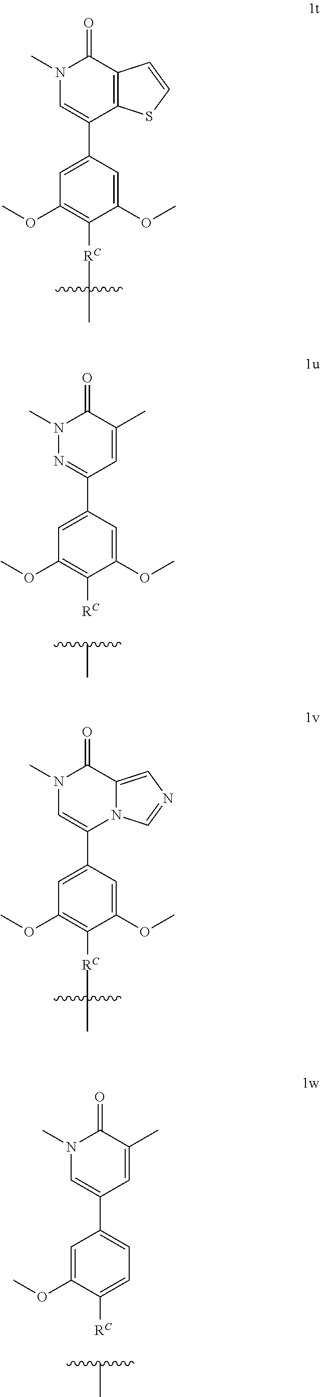 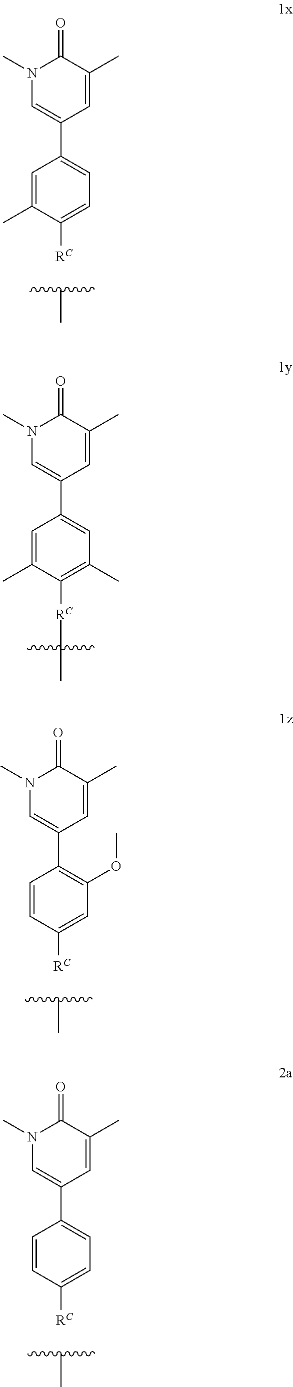 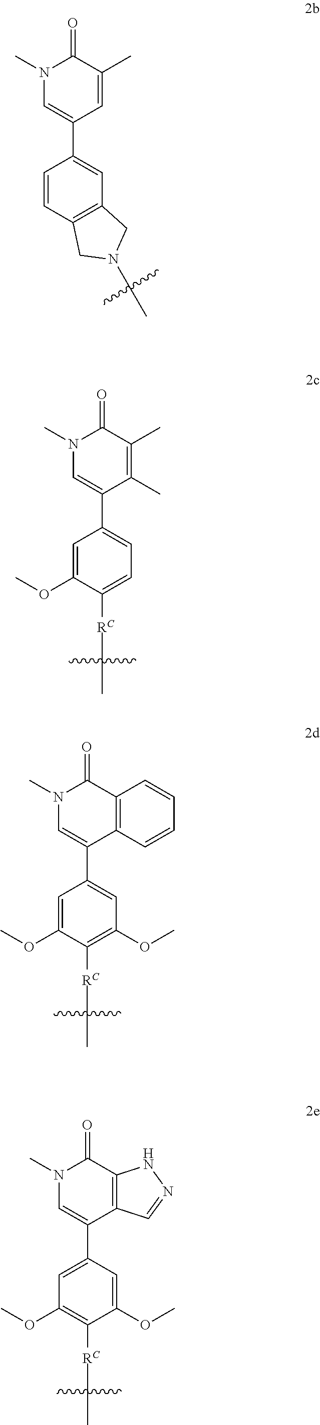 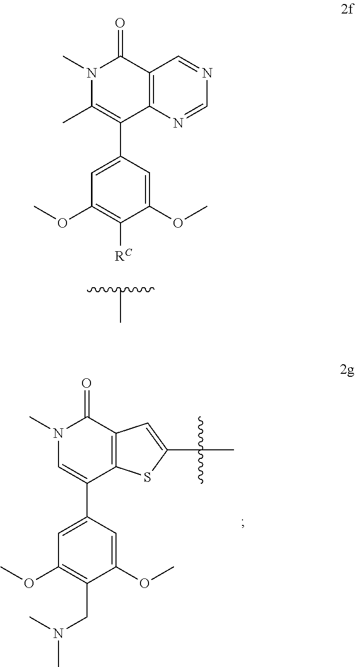
wherein R^{C} is absent, or is —Y—R^{G};
Y is selected from the group consisting of —CR^{H}R^{I}—, and —CO—;
R^{H} and R^{I} are each —H; or R^{H} and R^{I} taken together form a —C₃₋₄cycloalkyl;
R^{G} is selected from the group consisting of —N(R^{J}R^{K}), N(C₁₋₃alkyl)(optionally substituted 4- to 7-membered monocyclic heterocycloalkylene), or —N(C₁₋₃alkyl)(optionally substituted 7- to 12-membered bicyclic heterocycloalkylene)); —O—; optionally substituted 4- to 7-membered monocyclic heterocycloalkylene containing one or two N ring atoms;
and optionally substituted 7- to 12-membered bicyclic heterocycloalkylene containing one or two N ring atoms;
R^{J} and R^{K}are as defined in claim 1;
wherein the wavy line intersects the bond between the BRD9 binder and the linker.

15. A bifunctional molecule according to any one of claims 1 to 14, wherein R^{C} is present and is any one selected from: 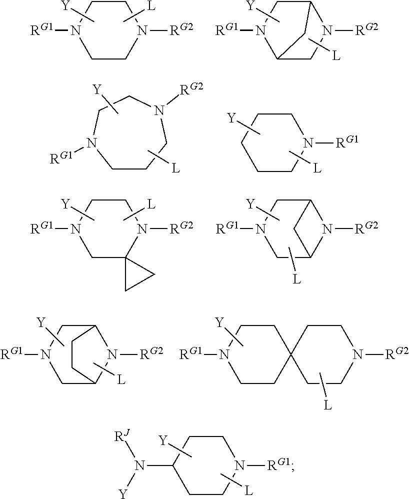
wherein Y is CR^{H}R^{I};
R^{G1} and R^{G2} are each independently selected from H and C₁-C₃ alkyl;
R^{J} is as defined in claim 1; and
L shows the point of attachment of the linker.

16. A bifunctional molecule according to any one of claims 1 to 15, wherein:
(i) when R¹ and R⁴ together form a 5-, 6-, or 7-membered heterocyclic ring, Z is represented by formula (Ia): 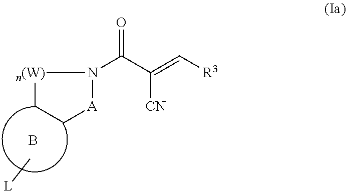
wherein A, B, R³ and L are as defined for formula (I); and
n is 1, 2 or 3;
W is selected from CR^{W1}R^{W2}, O, NR^{W3} and S;
R^{W1}, R^{W2} and R^{W3} are each independently selected from H and C₁ to C₆ alkyl; and wherein when n is 2 or 3, each W is independently selected from CR^{W1}R^{W2}, O, NR^{W3}, and S;
(ii) when R¹ and R² together form a 5-, 6-, or 7-membered heterocyclic ring, Z is represented as formula (Ib): 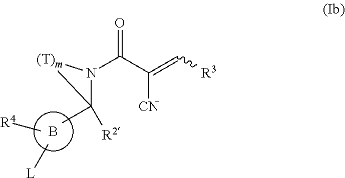
Wherein B, R^{2′}, R³, R⁴ and L are as defined for formula (I);
m is 3, 4 or 5;
each T is independently selected from CR^{T1}R^{T2}, O, NR^{T3} and S; and
R^{T1}, R^{T2} and R^{T3} are each independently selected from H and C₁ to C₆ alkyl; or
(iii) when R² and R⁴ together form a 5-, 8-, or 7-membered heterocyclic or carbocyclic ring, Z is represented as formula (Ic): 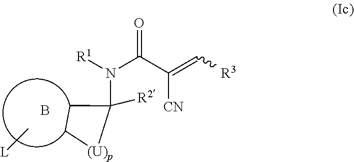
Wherein B, R¹, R^{2′}, R³ and L are as defined for formula (I);
p is 2, 3 or 4; and
each U is independently selected from CR^{U1}R^{U2}, O, NR^{U3} and S; and
R^{U1}, R^{U2} and R^{U3} are each independently selected from H and C₁ to C₆ alkyl.

17. The bifunctional molecule according to any one of the preceding claims, wherein R³ is selected from the group consisting of a heteroaryl, substituted heteroaryl, C₁-C₆ alkyl, C₃-C₆ cycloalkyl, C₃-C₆ cycloheteroalkyl, C₁-C₆ alkyl substituted with a heterocyclic group, aryl, and substituted aryl,
optionally wherein R³ is selected from: 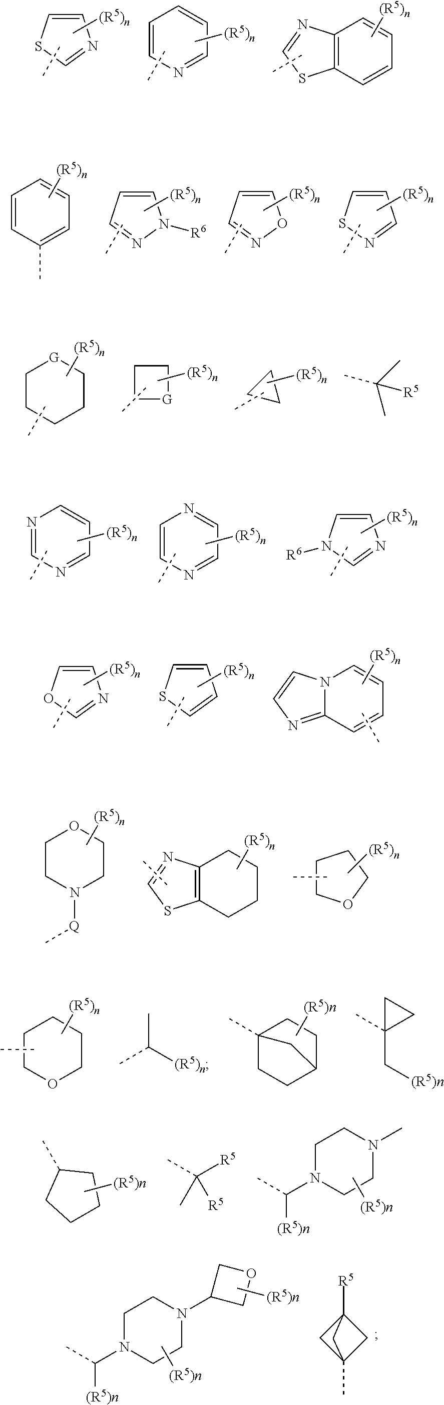
wherein the dotted line indicates the position at which each of the respective R³ groups is joined to the structure shown in formula (I) to (Ic), or wherein when the dotted line is not appended to an atom, the dotted line indicates that each of the respective R³ groups is joined to the structure via any position on the aromatic or heteroaromatic ring;
each R⁵ is independently selected from the group consisting of halo, CH₂OH, CF₃, —CH₂F, —CHF₂, OCF₃, —OCH₂F, —OCHF₂, C₁ to C₆ alkyl, —CN, —OH, —OMe, —SMe, —SOMe, —SO₂Me, —NH₂, —NHMe, —NMe₂, CO₂Me, —NO₂, CHO and COMe;
n is 0 to 3;
R⁶ is C₁ to C₆ alkyl;
G is CH₂, O and NH; and
Q is C₁ to C₆ alkylene.

18. The bifunctional molecule according to any one of the preceding claims, wherein A is CR²R^{2′}, optionally wherein: (i) one of R² and R^{2′} is a hydrogen and the other is C₁ to C₆ alkyl, optionally wherein the C₁ to C₆ alkyl is substituted with one or more halo atoms; or (ii) both of R² and R^{2′} are selected from C₁ to C₆ alkyl.

19. The bifunctional molecule according to any one of the preceding claims, wherein Z is represented as formula (IIaa): 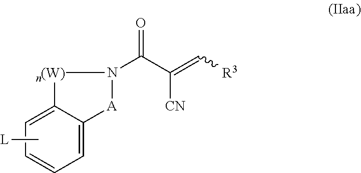
wherein A, R³, and L are as defined for formula (I);
n is 1, 2 or 3; and
W is selected from CR^{W1}R^{W2}, O, NR^{W3} and S; and
R^{W1}, R^{W2} and R^{W3} are each independently selected from H and C₁ to C₆ alkyl; and
wherein when n is 2 or 3, each W is independently selected from CR^{W1}R^{W2}, O, NR^{W3}, and S;
optionally wherein:
(i) Z is represented as formula (IIa): 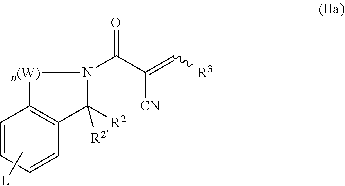
wherein R², R^{2′}, R³ and L are as defined in any one of the preceding claims,
n is 1, 2 or 3; and
W is selected from CR^{W1}R^{W2}, O, NR^{W3} and S; and
R^{W1}, R^{W2} and R^{W3} are each independently selected from H and C₁ to C₆ alkyl; and
wherein when n is 2 or 3, each W is independently selected from CR^{W1}R^{W2}, O, NR^{W3}, and S.

20. The bifunctional molecule according to any one of the preceding claims, wherein the linker comprises 1 to 25 or 1 to 18 atoms in a single linear chain.

21. The bifunctional molecule according to any one of the preceding claims, wherein linker comprises 1 to 10 or 1 to 8 rotatable bonds.

22. The bifunctional molecule according to any one of the preceding claims, wherein the linker (L) is a covalent bond or the structure of the linker (L) is:
(Lₓ)_{q}
wherein each Lx represents a subunit of L that is independently selected from CR^{L1}R^{L2}, O, C═O, S, SO, SO₂, NR^{L3}, SONR^{L4}, SONR^{L5}C═O, CONR^{L6}, NR^{L7}CO, C(R^{L8})═C(R^{L9}), C≡C, aryl, substituted aryl, heteroaryl, substituted heteroaryl, carbocyclyl, substituted carbocyclyl, heterocyclyl and substituted heterocyclyl groups;
wherein R^{L1}, R^{L2}, R^{L3}, R^{L4}, R^{L5}, R^{L6}, R^{L7}, R^{L8} and R^{L9} are each independently selected from H, halo, C₁ to C₆ alkyl, C₁ to C₆, haloalkyl, —OH, —O(C₁ to C₆ alkyl), —NH₂, —NH(C₁ to C₆ alkyl), —NO₂, —CN, —CONH₂, —CONH(C₁ to C₆ alkyl), —CON(C₁ to C₆ alkyl)₂, —SO₂(C₁ to C₆ alkyl), —CO₂(C₁ to C₆ alkyl), and —CO(C₁ to C₆ alkyl); and
q is an integer between 1 and 30.

23. The bifunctional molecule according to any one of claims 1 to 22, wherein the linker (L) may be represented as shown in formula (Lia): 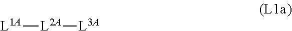
wherein L^{1A} is absent or is selected from C₁-C₆ alkylene, C₁-C₆ alkoxy and C₁-C₆ alkylamino;
L^{2A} is —NR^{L2A}C═O— or —C═ONR^{L2A}—; and
L^{3A} is selected from C₁-C₃ alkylene, C₁-C₆ alkoxy and C₁-C₆ alkylamino;
wherein R^{L2A} is H or C₁-C₆ alkyl);
or, the structure of the linker L may be represented as shown in formula (L1b): 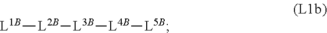
wherein L^{1B} is absent or is selected from C₁-C₃ alkylene, C₁-C₆ alkoxy and C₁-C₆ alkylamino;
L^{2B} is —NR^{L2A}C═O— or —C═ONR^{L2A}—;
L^{3B} is selected from C₁-C₁₅ alkylene, —[(CH₂)₂O]₁₋₆(CH₂)₂—;
L^{4B} is —NR^{L2A}C═O— or —C═ONR^{L2A}— wherein R^{L2A} is H or C₁-C₆ alkyl;
L^{5B} is selected from C₁-C₃alkylene, C₁-C₆ alkoxy and C₁-C₆ alkylamino;
wherein R^{L2A} is H or C₁-C₆ alkyl);
or, the structure of the linker (L) may be represented as shown in formula (L1c): 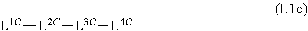
wherein L^{1C} is an optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from N, O and S;
L^{2C} is absent or is selected from C₁-C₃ alkylene, C₁-C₆ alkoxy and C₁-C₆ alkylamino;
L^{3C} is —R^{L2B}C═O— or —(C═O)R^{L2B}—; and
L^{4C} is selected from C₁-C₃alkylene, C₁-C₆ alkoxy and C₁-C₆ alkylamino;
wherein:
R^{L2A} is H or C₁-C₆ alkyl; and
R^{L2B} is NR^{L2A}; or an N-linked optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from N, O and S;
or, the structure of the linker (L) may be represented as shown in formula (L1d): 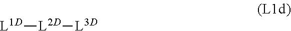
wherein L^{1D} is absent or is selected from C₁-C₃ alkylene, CO, C₁-C₃ alkylene(N(C₁-C₃ alkyl);
L^{2D} is NR^{L2A} or an optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from N, O and S; wherein R^{L2A} is H or C₁-C₆ alkyl; and
L^{3D} is absent or is selected from C₁-C₃ alkylene, —O—, —N(C₁-C₃ alkyl)-, and CO;
or, the structure of the linker (L) may be represented as shown in formula (L1e): 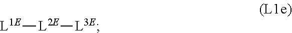
wherein L^{1E} is C₁-C₃ alkylene or CO;
L^{2E} is an optionally substituted 4- to 7-membered monocyclic N-heterocycloalkyl, an optionally substituted 7- to 12-membered bicyclic N-heterocycloalkyl, each optionally containing one or two additional ring heteroatoms selected from N, O and S; and
L^{3E} is selected from C₁-C₃ alkylene;
or, the linker (L) may be represented as shown in formula (L1f):
L^{1F} (L1f)
wherein L^{1F} is selected from C₁-C₃ alkylene, CO, and C₁-C₃ alkylene(NR^{L1C}); wherein
R^{L1C} is H or C₁-C₃ alkyl.

24. The bifunctional molecule according to any one of the preceding claims, wherein the bifunctional molecule has a structure as shown in Table 1.

25. A pharmaceutical composition comprising the bifunctional molecule according to any one of the preceding claims, together with a pharmaceutically acceptable carrier, optionally wherein the bifunctional molecule is present in the composition as a pharmaceutically acceptable salt, solvate or derivative.

26. The bifunctional molecule according to any one of claims 1 to 24 or the pharmaceutical composition of claim 25, for use in medicine.

27. The bifunctional molecule or pharmaceutical composition for use of claim 26, wherein the use comprises the treatment and/or prevention of any disease or condition which is associated with and/or is caused by an abnormal level of BRD9 activity.

28. The bifunctional molecule or pharmaceutical composition for use of claim 26 or 27, wherein the disease or condition is cancer.

29. A method of selectively degrading and/or increasing proteolysis of BRD9 in a cell, the method comprising contacting and/or treating the cell with a bifunctional molecule as defined in any one of claims 1 to 24 or a pharmaceutical composition as defined in claim 25.

30. A method of making a bifunctional molecule as defined in any one of claims 1 to 24.

31. A method of screening the bifunctional molecules according to any one of claims 1 to 24, comprising:
a. providing a bifunctional molecule comprising:
(i) a first ligand comprising a structure according to Z as defined in any one of claims 1 and 16 to 19;
(ii) a second ligand that binds to BRD9 as defined in any one of claims 1 to 15; and
(iii) a linker that covalently attaches the first and second ligands as defined in any one of claims 1 and 20 to 23;
b. contacting a cell with the bifunctional molecule;
c. detecting degradation of BRD9 in the cell;
d. detecting degradation of BRD9 in the cell in the absence of the bifunctional molecule; and
e. comparing the level of degradation of BRD9 in the cell contacted with the bifunctional molecule to the level of degradation of BRD9 in the absence of the bifunctional molecule;
wherein an increased level of degradation of BRD9 in the cell contacted with the bifunctional molecule indicates that the bifunctional molecule has facilitated and/or promoted the degradation of BRD9,
optionally wherein detecting degradation of BRD9 comprises detecting changes in the levels of the target protein in the cell.

32. A compound library comprising a plurality of bifunctional molecules according to any one of claims 1 to 24.

33. A compound library comprising a plurality of TBL or L or Z portions of bifunctional molecules according to any one of claims 1 to 24.
